(19) United States
(12) Patent Application Publication
(10) Pub. No.: US 2026/0167657 A1
(43) Pub. Date: Jun. 18, 2026

(54) AMINO-IMIDAZOLE ANTIBACTERIAL COMPOUNDS

(71) Applicant: Hoffmann-La Roche Inc., Little Falls, NJ (US)

(72) Inventors: Jean-Baptiste Emmanuel BLANC, Westfield, NJ (US); Patrick DI GIORGIO, Riehen (CH); Christian KRAMER, Lörrach (DE); Petra SCHMITZ, Kleines Wiesental (DE); Theodor Walter Jakob STOLL, Binningen (CH); Lorenz Michael URNER, Basel (CH); William John ZUERCHER, Mies (CH)

(73) Assignee: Hoffmann-La Roche Inc., Little Falls, NJ (US)

(21) Appl. No.: 19/364,955

(22) Filed: Oct. 21, 2025

(30) Foreign Application Priority Data
Oct. 22, 2024 (EP) 24207990.3

(51) Int. Cl. C07F 9/6558; A61K 31/4178; A61K 31/454; A61K 31/496; A61K 31/4985; A61K 31/499; A61K 31/4995; A61K 31/506; A61K 31/519; A61K 31/527; A61K 31/5377; A61K 31/5386; A61K 31/541; A61K 31/551; A61K 31/554; A61K 31/675; A61P 31/04; C07D 233/90; C07D 401/12; C07D 401/14; C07D 403/04; C07D 403/12; C07D 403/14; C07D 405/12; C07D 405/14; C07D 409/12; C07D 413/12; C07D 413/14; C07D 417/14; C07D 471/04; C07D 471/08; C07D 471/10; C07D 487/04; C07D 487/08; C07D 487/10; C07D 491/048; C07D 491/107; C07D 495/10; C07D 498/04; C07D 498/08; C07D 498/10; C07F 9/6584

(52) U.S. Cl. CPC C07F 9/65583; A61K 31/4178; A61K 31/454; A61K 31/496; A61K 31/4985; A61K 31/499; A61K 31/4995; A61K 31/506; A61K 31/519; A61K 31/527; A61K 31/5377; A61K 31/5386; A61K 31/541; A61K 31/551; A61K 31/554; A61K 31/675; A61P 31/04; C07D 233/90; C07D 401/12; C07D 401/14; C07D 403/04; C07D 403/12; C07D 403/14; C07D 405/12; C07D 405/14; C07D 409/12; C07D 413/12; C07D 413/14; C07D 417/14; C07D 471/04; C07D 471/08; C07D 471/10; C07D 487/04; C07D 487/08; C07D 487/10; C07D 491/048; C07D 491/107; C07D 495/10; C07D 498/04; C07D 498/08; C07D 498/10; C07F 9/6584

(57) ABSTRACT

Provided herein are amino imidazole compounds useful in treating infections.

This application claims benefit of priority to European Application No. 24207990.3, filed Oct. 22, 2024, which is incorporated herein by reference in its entirety.

The present invention relates to amino-imidazole derivatives that exhibit antibacterial properties. The invention also relates to methods of using the compounds for the treatment or prevention of bacterial infections and resulting diseases, in particular for the treatment or prevention of infections with Acinetobacter baumannii and resulting diseases.

### BACKGROUND

Acinetobacter baumannii is a Gram-negative, aerobic, nonfermenting bacterium recognized over the last decades as an emergining pathogen with very limited treatment options.

A. baumannii is considered to be a serious threat by the US Centers for Disease Control and Prevention and belongs to the so called ‘ESKAPE’ pathogens (Enterococcus faecium, Staphylococcus aureus, Klebsiella pneumoniae, Acinetobacter baumannii, Pseudomonas aeruginosa and Enterobacter species & E. coli) that currently cause the majority of nosocomial infections and effectively “escape” the activity of antimicrobial agents.

A. baumannii is most often encountered in intensive care units and surgical wards, where extensive antibiotic use has enabled selection for resistance against all known antimicrobials and where it causes infections that include bacteremia, pneumonia, meningitis, urinary tract infection, and wound infection.

A. baumannii has an exceptional ability to upregulate and acquire resistance determinants and shows an environmental persistance that allows its survival and spread in the nosocomial setting, making this organism a frequent cause of outbreaks of infection and an endemic, health care-associated pathogen.

Due to increasing antibiotic resistance to most if not all available therapeutic options, Muti-Drug Resistant (MDR) A. baumanniii infections, especially those caused by Carbapenem resistant A. baumannii, are extremely difficult or even impossible to treat with high mortality rate as well as increased morbidity and length of stay in intensive care unit.

Acinetobacter baumannii has been defined and still remains “a prime example of a mismatch between unmet medical needs and the current antimicrobial research and development pipeline” according to the Antimicrobial Availability Task Force (AATF) of the Infectious Diseases Society of America (IDSA). Thus, there is a high demand and need to identify compounds suitable for the treatment of diseases and infections caused by Acinetobacter baumannii.

Accordingly, there is a pressing need for agents that exhibit activity against drug-susceptible as well as drug-resistant strains of Acinetobacter baumannii.

### SUMMARY

Provided herein are solutions to the problems above and other problems in the art.

In a first aspect provided herein is a compound of formula (I) or (II) as described herein.

In a second aspect provided herein is a compound of formula (I) as described herein.

In a third aspect provided herein is a compound of formula (1a), (1b), or (1c) as described herein.

In a fourth aspect provided herein is a compound of formula (1d) as described herein.

In a fifth aspect provided herein is a compound of formula (1e) as described herein.

In a sixth aspect provided herein is a compound of Table 1 as set forth herein.

In a seventh aspect provided herein is a pharmaceutical composition comprising a compound or a pharmaceutically acceptable salt thereof as described herein.

In an eighth aspect provided herein is a compound or a pharmaceutically acceptable salt thereof as described herein, for use in the treatment of infection caused by Gram-negative bacteria.

In a ninth aspect provided herein is a compound or a pharmaceutically acceptable salt thereof as described herein, for use in the treatment of infections caused by Enterococcus faecium, Staphylococcus aureus, Klebsiella pneumoniae, Acinetobacter baumannii, Pseudomonas aeruginosa, Enterobacter species or E. coli, or a combination thereof.

In a tenth aspect provided herein is a method of treating an infection caused by Gram-negative bacteria in a patient having such an infection, the method comprising administering to the patient an effective amount of a compound or a stereoisomer, tautomer, or pharmaceutically acceptable salt thereof as described herein.

In an eleventh aspect provided herein is a method of treating an infection caused by Enterococcus faecium, Staphylococcus aureus, Klebsiella pneumoniae, Acinetobacter baumannii, Pseudomonas aeruginosa, Enterobacter species or E. coli, or a combination thereof, in a patient having such infection, the method comprising administering to the patient a compound or a stereoisomer, tautomer or pharmaceutically acceptable salt thereof described herein.

### DETAILED DESCRIPTION

### Definitions:

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process disclosed, may be combined in any combination, except combinations where at least some of such features and/or steps are mutually exclusive.

Unless otherwise defined, all technical and scientific terms used herein have the same meaning as commonly understood by one of ordinary skill in the art. All publications, patent applications, patents, and other references mentioned herein are incorporated by reference in their entirety. The nomenclature used in this Application is based on IUPAC systematic nomenclature, unless indicated otherwise.

The following definitions are provided to facilitate understanding of certain terms used frequently herein and are not meant to limit the scope of the present disclosure. All references referred to herein are incorporated by reference in their entirety.

The term “alkyl” refers to a mono- or multivalent, e.g., a mono- or bivalent, linear or branched saturated hydrocarbon group of 1 to 6 carbon atoms (“C₁-C₆-alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl.

The term “alkenyl” refers to refers to linear or branched-chain monovalent hydrocarbon radical with at least one carbon-carbon double bond, and includes radicals having “cis” and “trans” orientations, or alternatively, “E” and “Z” orientations. In one example, the alkenyl radical is two to eighteen carbon atoms (C₂₋₁₈). In other examples, the alkenyl radical is C₂₋₁₂, C₂₋₁₀, C₂₋₈, C₂₋₆, or C₂₋₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl.

The term “alkynyl” refers to refers to a linear or branched monovalent hydrocarbon radical with at least one carbon-carbon, triple bond. In one example, the alkynyl radical is two to eighteen carbon atoms (C₂₋₁₈). In other examples, the alkynyl radical is C₂₋₁₂, C₂₋₁₀, C₂₋₈, C₂₋₆, or C₂₋₃. Examples include, but are not limited to, ethynyl (—C≡CH), prop-1-ynyl (—C≡CCH₃), prop-2-ynyl (propargyl, —CH₂C≡CH), but-1-ynyl, but-2-ynyl, and but-3-ynyl.

The term “alkoxy” refers to an alkyl group, as previously defined, attached to the parent molecular moiety via an oxygen atom. Unless otherwise specified, the alkoxy group contains 1 to 6 carbon atoms (“C₁-C₆-alkoxy”). In some preferred embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy.

The term “haloalkyl” refers to an alkyl chain in which one or more hydrogen has been replaced by a halogen. Examples of haloalkyls are trifluoromethyl, difluoromethyl, and fluoromethyl. A substituted haloalkyl refers to a haloalkyl having a moiety other than a halogen.

The terms “halogen” or “halo” are used interchangeably herein and refer to fluoro (F), chloro (Cl), bromo (Br), or iodo (I).

The term “alkylheterocyclyl” is used herein and refers to an alkyl group, wherein at least one of the hydrogen atoms of the alkyl group has been replaced by a heterocyclyl moiety. In one embodiment, “alkyl heterocyclyl” refers to an alkyl group wherein 1, 2 or 3 hydrogen atoms of the alkyl group have been replaced by a heterocyclyl moiety. In another embodiment, “alkyl heterocyclyl” and “heterocyclylalkyl” refers to an alkyl group wherein 1 hydrogen atom of the alkyl group has been replaced by a heterocyclyl moiety.

The term “cycloalkyl” as used herein refers to a saturated or partly unsaturated monocyclic or bicyclic hydrocarbon group of 3 to 10 ring carbon atoms (“C₃₋₁₀-cycloalkyl”). In some embodiments, the cycloalkyl group is a saturated monocyclic hydrocarbon group of 3 to 6 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Some non-limiting examples of cycloalkyl include cyclopropyl, cyclobutyl, cyclopentyl, cyclohexyl, cycloheptyl, 1-bicyclo[1.1.1]pentanyl, norbornanyl, and 1-bicyclo[2.2.2]octanyl.

The term “heterocyclyl” refers to a saturated or partly unsaturated mono- or bicyclic, monocyclic ring system of 3 to 10 ring atoms, 3 to 8 ring atoms, or 3 to 6 ring atoms, wherein 1, 2, or 3 of the ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidine, morpholine, thiomorpholine, piperazine, pyrrolidinyl, piperidyl, pyridyl, cyclopropyl, cyclopentyl, 1,2,3,3a,4,5,6,6a-octahydropyrrolo[3,4-c]pyrrole, 9-oxa-3,7-diazabicyclo[3.3.1]nonane, 7-oxa-2-azaspiro[3.5]nonane, and 2,6-dioxa-9-azaspiro[4.5]decane.

The term “alkylaryl” refers to an alkyl group, wherein at least one of the hydrogen atoms of the alkyl group has been replaced by an aryl moiety. In one embodiment, “alkylaryl” refers to an alkyl group wherein 1, 2 or 3 hydrogen atoms of the alkyl group have been replaced by an aryl moiety. In another embodiment, “alkylaryl” refers to an alkyl group wherein 1 hydrogen atom of the alkyl group has been replaced by an aryl moiety.

The term “aryl” refers to a monocyclic, bicyclic, or tricyclic carbocyclic ring system having a total of 6 to 14 ring members (“C₆-C₁₄-aryl”), 6 to 12 ring members, or 6 to 10 ring members, and wherein at least one ring in the system is aromatic.

Some non-limiting examples of aryl include phenyl and 9H-fluorenyl (e.g. 9H-fluoren-9-yl).

The term “hydroxy” refers to an —OH group.

The term “amino” refers to an —NH₂ group.

The term “carboxy” refers to a group —COOH.

The term “cyano” and “—CN” refer to a nitrile group.

The term “oxo” refers to an ═O group.

The term “imino” refers to an ═NH group.

The term “carbamoyl” refers to a group —C(O)—NH₂.

The term “hydroxycarbamoyl” refers to a group —C(O)—NH—OH.

As used herein a wavy line “ [symbol] ” that intersects a bond in a chemical structure indicate the point of attachment of the atom to which the wavy bond is connected in the chemical structure to the remainder of a molecule, or to the remainder of a fragment of a molecule.

In certain embodiments, divalent groups are described generically without specific bonding configurations. It is understood that the generic description is meant to include both bonding configurations, unless specified otherwise. For example, in the group R¹—R²—R³, if the group R² is described as —CH₂C(O)—, then it is understood that this group can be bonded both as R¹—CH₂C(O)—R³, and as R¹—C(O)CH₂—R³, unless specified otherwise.

The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, trifluoroacetic acid, propionic acid, glycolic acid, pyruvic acid, oxalic acid, maleic acid, malonic acid, succinic acid, fumaric acid, tartaric acid, lactic acid, citric acid, benzoic acid, cinnamic acid, mandelic acid, methanesulfonic acid, ethanesulfonic acid, p-toluenesulfonic acid, salicylic acid, n-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, n-ethylpiperidine, piperidine, polyimine resins and the like. Particular pharmaceutically acceptable salts of compounds of formula (I) are hydrochlorides, fumarates, lactates (in particular derived from L-(+)-lactic acid), tartrates (in particular derived from L-(+)-tartaric acid) and trifluoroacetates.

The term “stereoisomers” refer to compounds that have identical chemical constitution, but differ with regard to the arrangement of the atoms or groups in space. Stereoisomers include diastereomers, enantiomers, atropisomers, conformers and the like.

The term “chiral” refers to molecules that have the property of non-superimposability of the mirror image partner, while the term “achiral” refers to molecules that are superimposable on their mirror image partner.

The term “diastereomer” refers to a stereoisomer with two or more centers of chirality and whose molecules are not mirror images of one another. Diastereomers have different physical properties, e.g., melting points, boiling points, spectral properties or biological activities. Mixtures of diastereomers may separate under high resolution analytical procedures such as electrophoresis and chromatography such as HPLC.

The term “enantiomers” refers to two stereoisomers of a compound that are non-superimposable mirror images of one another.

In the structures shown herein, where the stereochemistry of any particular chiral atom is not specified, then all stereoisomers are contemplated and included. Where stereochemistry is specified by a solid wedge or dashed line representing a particular configuration, then that stereoisomer is so specified and defined. Unless otherwise specified, if solid wedges or dashed lines are used, relative stereochemistry is intended.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Certain compounds described herein can exist in unsolvated forms as well as solvated forms, including hydrated forms. A “solvate” refers to an association or complex of one or more solvent molecules and a compound described herein. Examples of solvents that form solvates include water, isopropanol, ethanol, methanol, DMSO, ethyl acetate, acetic acid, and ethanolamine. Certain compounds described herein can exist in multiple crystalline or amorphous forms. In general, all physical forms are contemplated herein. The term “hydrate” refers to the complex where the solvent molecule is water.

The term “co-administering” refers to either simultaneous administration, or any manner of separate sequential administration, of a compound, stereoisomer, tautomer, or a pharmaceutically acceptable salt thereof as described herein and a further active pharmaceutical ingredient or ingredients, including antibiotic agents. If the administration is not simultaneous, the agents are administered in a close time proximity to each other.

The term “effective amount” is at least the minimum amount required to effect a measurable improvement or prevention of a disease described herein. An effective amount herein may vary according to factors such as the disease state, age, sex, and weight of the patient, combination with other agents, and the ability of the agent to elicit a desired response in the patient. An effective amount is also one in which any toxic or detrimental effects of the treatment are outweighed by the therapeutically beneficial effects. Beneficial or desired results include results such as eliminating or reducing the risk, lessening the severity, delaying the onset of the disease (including biochemical, histological and/or behavioral symptoms of the disease, its complications and intermediate pathological phenotypes presenting during development of the disease), decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount can be administered in one or more administrations.

The term “nosocomial infection” refers to a hospital-acquired infection (HAI), which is an infection that is acquired in a hospital or other health care facility. To emphasize both hospital and nonhospital settings, it is sometimes instead called a health care-associated infection (HAI or HCAI). Such an infection can be acquired in hospitals, nursing homes, rehabilitation facilities, outpatient clinics, or other clinical settings.

The term “package insert” is used to refer to instructions customarily included in commercial packages of therapeutic products that contain information about the indications, usage, dosage, administration, contraindications and/or warnings concerning the use of such therapeutic products.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, trifluoroacetic acid, propionic acid, glycolic acid, pyruvic acid, oxalic acid, maleic acid, malonic acid, succinic acid, fumaric acid, tartaric acid, lactic acid, citric acid, benzoic acid, cinnamic acid, mandelic acid, methanesulfonic acid, ethanesulfonic acid, p-toluenesulfonic acid, salicylic acid, n-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, n-ethylpiperidine, piperidine, polyimine resins and the like. Particular pharmaceutically acceptable salts of compounds of formula (I) are hydrochlorides, fumarates, lactates (in particular derived from L-(+)-lactic acid), tartrates (in particular derived from L-(+)-tartaric acid) and trifluoroacetates.

The terms “prophylaxis” and “prevention” are used interchangeably herein and as used herein includes preventing or delaying the appearance of clinical symptoms of the state, disorder or condition developing in a patient who may be afflicted with or predisposed to the state, disorder or condition but does not yet experience or display clinical or subclinical symptoms of the state, disorder or condition. In a one embodiment, the term “patient” refers to a human.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

It is specifically contemplated that any limitation discussed with respect to one embodiment provided herein may apply to any other embodiment provided herein. Furthermore, any compound and pharmaceutically acceptable salts thereof described herein or composition described herein may be used in any method provided herein, and any method provided herein may be used to produce or to utilize any compound and pharmaceutically acceptable salts thereof described herein or composition described herein.

### Compounds

Provided herein are compounds of formula (I) or (II)

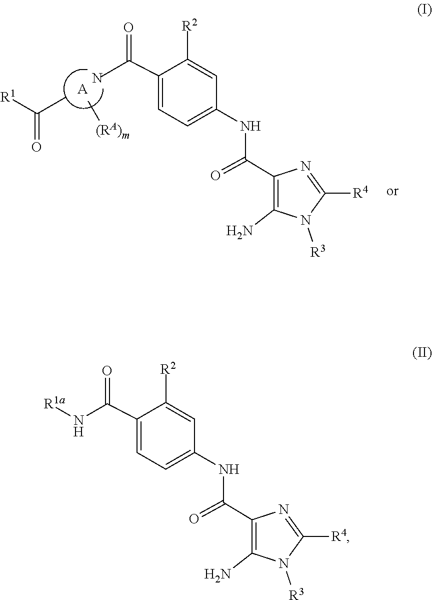

or a stereoisomer or pharmaceutically acceptable salt thereof, wherein
Ring A is 4- to 8-membered R^{A}-substituted or unsubstituted heterocyclyl;
R^{A} is halogen, NH₂, OH, unsubstituted C₁₋₃alkyl, or unsubstituted C₁₋₃haloalkyl;
m is 0 or 1;
R¹ is N(R⁶)₂, R⁶-substituted or unsubstituted C₁₋₆alkyl, R⁶-substituted or unsubstituted C₁₋₆haloalkyl, R⁶-substituted or unsubstituted C₃₋₆cycloalkyl, or R⁶-substituted or unsubstituted 4- to 8-membered heterocyclyl comprising 1 or 2 heteroatoms independently selected from N, O, S, or SO₂;
each R⁶ is independently hydrogen, halogen, CH(═NH)(NH₂), (═NH), N(R⁷)₂, OR⁷, NHC(O)R⁷, C(O)OR⁷, R⁷-substituted or unsubstituted C₁₋₆alkyl, R⁷-substituted or unsubstituted C₁₋₆haloalkyl, R⁷-substituted or unsubstituted C₃₋₆ cycloalkyl, or R⁷-substituted or unsubstituted 4- to 6-membered heterocyclyl; or
wherein two or more R⁶ groups together form a C₁₋₂ bridge;
each R⁷ is hydrogen, halo, OH, COOH, (═NH), NH₂, unsubstituted C₁₋₃alkyl, unsubstituted C₁₋₃alkyoxy, unsubstituted C₁₋₃alkylaryl, unsubstituted C₁₋₆alkyl heterocyclyl, or unsubstituted 4- to 6-membered heterocyclyl;
R^{1a} is a moiety of formula:

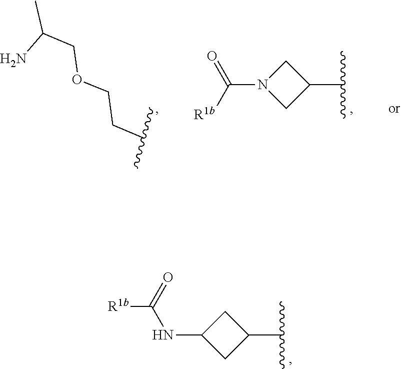

or a stereoisomer thereof;
R^{1b} is O—C₁₋₃alkyaryl, unsubstituted 5- to 7-membered heterocyclyl;
R² is hydrogen, halogen, unsubstituted C₁₋₃alkyl, or unsubstituted C₁₋₃haloalkyl;
R³ is a moiety of formula:

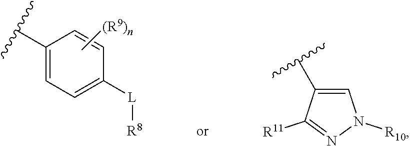

or a stereoisomer thereof
L is absent, —O—, or —NR^{8b}—;
R⁸ is R^{8a}-substituted or unsubstituted C₁₋₆alkyl, R^{8a}-substituted or unsubstituted C₁₋₃haloalkyl, or R^{8a}-substituted or unsubstituted C₃₋₆cycloalkyl;
wherein when L is absent R⁸ is

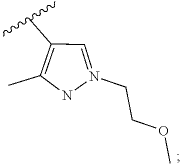

R^{8a} is unsubstituted C₁₋₃alkyl, OH, or OR^{8b} wherein R^{8b} is unsubstituted C₁₋₃alkyl;
R^{8b} is hydrogen or unsubstituted C₁₋₃alkyl;
R⁹ is halo, CN, unsubstituted C₁₋₆alkyl, unsubstituted C₁₋₃haloalkyl, unsubstituted C₁₋₆alkoxy, or unsubstituted cyclopropyl;
n is 0, 1, or 2;
R¹⁰ is R^{10A}-substituted or unsubstituted C₁₋₆alkyl, R^{10A}-substituted or unsubstituted C₂₋₆alkenyl, R^{10A}-substituted or unsubstituted C₂₋₆alkynl, R^{10A}, substituted or unsubstituted C₁₋₃haloalkyl, or R^{10A}-substituted or unsubstituted C₃₋₆cycloalkyl;
R^{10A} is halo, CN, OH, OCH₃, unsubstituted C₁₋₃haloalkyl, or R^{10B}-substituted or unsubstituted C₃₋₅cycloalkyl, or wherein 2 R^{10A} groups together form a R^{10B}-substituted or unsubstituted C₃₋₄spirocycle;
R¹¹ is halo or unsubstituted C₁₋₃haloalkyl; and
R⁴ is hydrogen, halo, or unsubstituted C₁₋₃alkyl.

In one embodiment of the compounds described herein, the compound comprises formula (I)

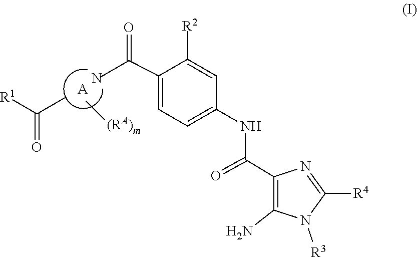

where Ring A is 4- to 8-membered R^{A}-substituted or unsubstituted heterocyclyl;
R^{A} is halogen, NH₂, OH, unsubstituted C₁₋₃alkyl, or unsubstituted C₁₋₃haloalkyl;
m is 0 or 1;
R¹ is N(R⁶)₂, OR⁶, R⁶-substituted or unsubstituted C₁₋₆alkyl, R⁶-substituted or unsubstituted C₁₋₃haloalkyl, R⁶-substituted or unsubstituted C₃₋₆cycloalkyl, or R⁶-substituted or unsubstituted 4- to 9-membered heterocyclyl comprising 1 or 2 heteroatoms independently selected from N, O, S, or SO₂;
each R⁶ is independently hydrogen, halogen, CN, oxo, C(═NH)(NH₂), C(═NH)R⁷, (═NH), N(R⁷)₂, OR⁷, NHC(O)R⁷, C(O)OR⁷, S(O)(═NH)R⁷, R⁷-substituted or unsubstituted C₁₋₆alkyl, R⁷-substituted or unsubstituted C₁₋₆haloalkyl, R⁷-substituted or unsubstituted C₃₋₆ cycloalkyl, or R⁷-substituted or unsubstituted 4- to 6-membered heterocyclyl; or
wherein two or more R⁶ groups together form a C₁₋₂ bridge;
each R⁷ is hydrogen, halo, SO₂CH₃, OR^{7a}, COOH, (CH₂)ᵣOH, CH(CH₂OH)₂, (═NH), NH₂, R^{7a}-substituted or unsubstituted C₁₋₃alkyl, unsubstituted C₁₋₃alkyoxy, unsubstituted C₁₋₃alkylaryl, unsubstituted C₁₋₆alkylheterocyclyl, or unsubstituted 4- to 8-membered heterocyclyl;
each R^{7a} is independently SO₂CH₃, SO₂CH₃, OH, (CH₂)ᵣOH, NH₂, NHCH₃, N(CH₃)₂, NHSO₂CH₃, unsubstituted C₁₋₃ alkyl, unsubstituted oxetanyl, unsubstituted oxooxazolidinyl, unsubstituted imidazolyl, unsubstituted pyrrolidinyl, unsubstituted piperidinyl, or unsubstituted tetrahydropyrimidinyl;
r is 1, 2, or 3;
R² is hydrogen, halogen, unsubstituted C₁₋₃alkyl, or unsubstituted C₁₋₃haloalkyl;
R³ is a moiety of formula:

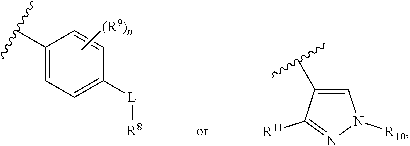

or a stereoisomer thereof
L is absent, —O—, or —NR^{8b}—;
R⁸ is R^{8a}-substituted or unsubstituted C₁₋₆alkyl, R^{8a}-substituted or unsubstituted C₁₋₃haloalkyl, or R^{8a}-substituted or unsubstituted C₃₋₆cycloalkyl;
wherein when L is absent R⁸ is

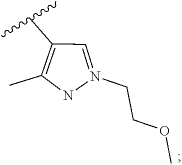

R^{8a} is unsubstituted C₁₋₃alkyl, OH, or OR^{8b} wherein R^{8b} is unsubstituted C₁₋₃alkyl;
R^{8b} is hydrogen or unsubstituted C₁₋₃alkyl;
R⁹ is halo, CN, unsubstituted C₁₋₆alkyl, unsubstituted C₁₋₃haloalkyl, unsubstituted C₁₋₆alkoxy, or unsubstituted cyclopropyl;
n is 0, 1, or 2;
R¹⁰ is R^{10A}-substituted or unsubstituted C₁₋₆alkyl, R^{10A}-substituted or unsubstituted C₂₋₆alkenyl, R^{10A}-substituted or unsubstituted C₂₋₆alkynl, R^{10A}-substituted or unsubstituted C₁₋₃haloalkyl, or R^{10A}-substituted or unsubstituted C₃₋₆cycloalkyl;
R^{10A} is halo, CN, OH, OCH₃, unsubstituted C₁₋₃haloalkyl, or R^{10B}-substituted or unsubstituted C₃₋₅cycloalkyl, or wherein 2 R^{10A} groups together form a R^{10B}-substituted or unsubstituted C₃₋₄spirocycle;
R¹¹ is halo or unsubstituted C₁₋₃haloalkyl; and
R⁴ is hydrogen, halo, or unsubstituted C₁₋₃alkyl.

In one embodiment of the compounds of formula (I) described herein, each R⁶ is independently hydrogen, halogen, C(═NH)(NH₂), (═NH), N(R⁷)₂, OR⁷, NHC(O)R⁷, C(O)OR⁷, R⁷-substituted or unsubstituted C₁₋₆alkyl, R⁷-substituted or unsubstituted C₁₋₆haloalkyl, R⁷-substituted or unsubstituted C₃₋₆ cycloalkyl, or R⁷-substituted or unsubstituted 4- to 6-membered heterocyclyl.

In one embodiment of the compounds of formula (I) described herein, each R⁷ is hydrogen, halo, OH, COOH, (═NH), NH₂, unsubstituted C₁₋₃alkyl, unsubstituted C₁₋₃alkyoxy, unsubstituted C₁₋₃alkylaryl, unsubstituted C₁₋₆alkylheterocyclyl, or unsubstituted 4- to 8-membered heterocyclyl.

In one embodiment of the compounds of formula (I) described herein, R¹ is N(R⁶)₂, R⁶-substituted or unsubstituted C₁₋₆alkyl, R⁶-substituted or unsubstituted C₁₋₃haloalkyl, R⁶-substituted or unsubstituted C₃₋₆cycloalkyl, or R⁶-substituted or unsubstituted 4- to 8-membered heterocyclyl comprising 1 or 2 heteroatoms independently selected from N, O, S, or SO₂.

In one embodiment of the compounds of formula (I) described herein, Ring A is 4- to 8-membered R^{A}-substituted or unsubstituted heterocyclyl. In one embodiment, Ring A is 4- to 6-membered R^{A}-substituted or unsubstituted heterocyclyl. In another embodiment, Ring A is a 6-membered R^{A}-substituted or unsubstituted heterocyclyl. In such embodiments, the heterocyclyl comprises 1 or 2 heteroatoms independently selected from N, O, or S. In such embodiments, the heterocyclyl comprises 1 or 2 ring N heteroatoms.

In one embodiment of the compounds of described herein, Ring A is a 4- to 6-membered R^{A}-substituted or unsubstituted heterocyclyl comprising 1 or 2 ring N heteroatoms. In one such embodiment, Ring A is unsubstituted. In another embodiment, Ring A is R^{A}-substituted or unsubstituted azetidinyl, pyrrolidinyl, pyrazolidinyl, or imidazolidinyl. In another embodiment, Ring A is R^{A}-substituted or unsubstituted piperidinyl or piperazinyl. In one such embodiment, Ring A is unsubstituted piperidinyl or unsubstituted piperazinyl. For orientation of Ring A relative to the compounds of formula (I), the C(O)R¹ group has been included in the following structures.

In one embodiment, Ring A is a moiety of formula:

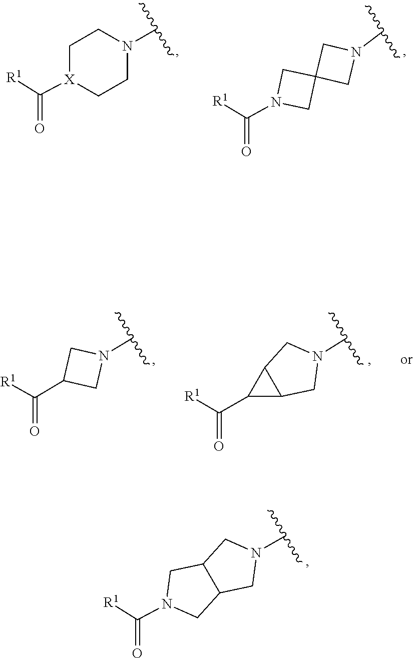

or a stereoisomer thereof, where
X is N or CR^{x};
R^{x} is hydrogen or halo; and
p is an integer of 0, 1, 2, 3, or 4.

In one embodiment, Ring A is a moiety of formula:

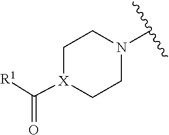

where R¹ is as described herein and X is N or CR^{x}. In one embodiment, X is N, CH or CF. In another embodiment, X is N or CH. In one such embodiment, X is N. In one such embodiment, X is CR^{x}. In one such embodiment, X is CH.

In another embodiment, Ring A is a moiety of formula:

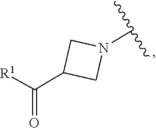

where R¹ is as described herein.

In another embodiment, Ring A is a moiety of formula:

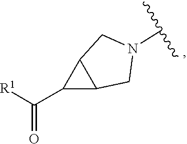

where R¹ is as described herein.

In another embodiment, Ring A is a moiety of formula:

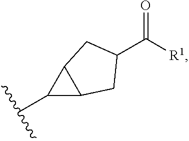

wherein R¹ is as described herein.

In one such embodiment, Ring A is a moiety of formula:

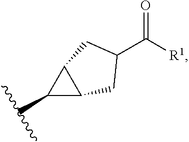

wherein R¹ is as described herein.

In one such embodiment, Ring A is a moiety of formula:

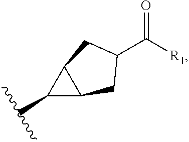

wherein R¹ is as described herein.

In another embodiment, Ring A is a moiety of formula:

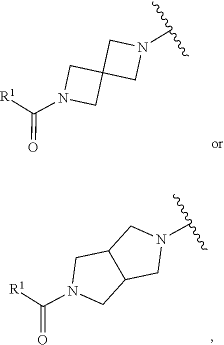

where R¹ is as described herein.

In some embodiments, the compound of formula (I) comprises a compound of formula:

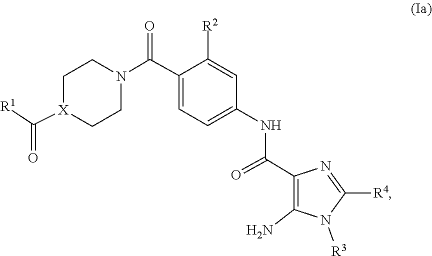

or a stereoisomer or pharmaceutically acceptable salt thereof, wherein
X is N, CH or CF, and R¹, R², R³, and R⁴ are as described herein.

In some embodiments, the compound of formula (I) comprises a compound of formula:

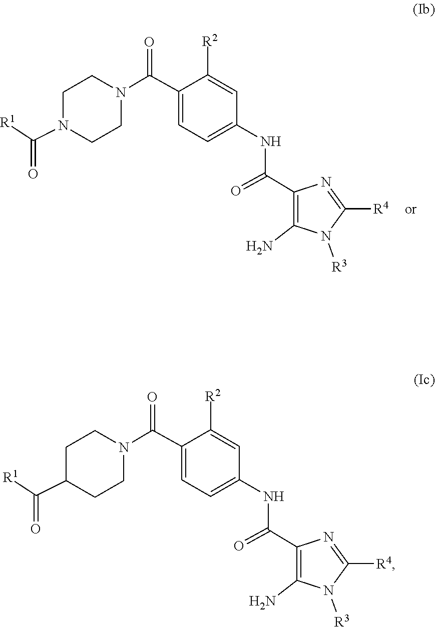

or a stereoisomer or pharmaceutically acceptable salt thereof, where R¹, R², R³, and R⁴ are as described herein.

In one embodiment of the compounds described herein, R¹ is N(R⁶)₂, R⁶-substituted or unsubstituted C₁₋₆alkyl, or R⁶-substituted or unsubstituted C₁₋₃haloalkyl. In one such embodiment, R¹ is R⁶-substituted or unsubstituted C₁₋₆alkyl, or R⁶-substituted or unsubstituted C₁₋₃haloalkyl. In another embodiment of the compounds described herein, R¹ is R⁶-substituted or unsubstituted C₃₋₆cycloalkyl or R⁶-substituted or unsubstituted 4- to 8-membered heterocyclyl comprising 1 or 2 heteroatoms independently selected from N, O, S, or SO₂. In one embodiment of the compounds described herein, R¹ is R⁶-substituted or unsubstituted C₃₋₆cycloalkyl. In one such embodiment, R¹ is R⁶-substituted or unsubstituted C₅₋₆cycloalkyl. In one embodiment of the compounds described herein, R¹ is R⁶-substituted or unsubstituted 4- to 8-membered heterocyclyl comprising 1 or 2 heteroatoms independently selected from N, O, S, or SO₂. In one such embodiment, R¹ is R⁶-substituted or unsubstituted 4- to 8-membered heterocyclyl comprising 1 or 2 heteroatoms independently selected from N. In another such embodiment, R¹ is R⁶-substituted or unsubstituted 6- to 8-membered heterocyclyl comprising 1 or 2 heteroatoms independently selected from N. In another such embodiment, R¹ is R⁶-substituted or unsubstituted 4- to 8-membered heterocyclyl comprising 1 or 2 heteroatoms independently selected from N, or O.

In one embodiment of the compounds described herein, R¹ is a moiety of formula:

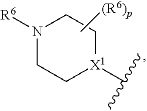

or a stereoisomer thereof, wherein X¹ is N or CH, each R⁶ is independently as described herein, and p is as described herein. In one such embodiment, X¹ is N. In another such embodiment, X¹ is CH.

In one embodiment of the compounds described herein, R⁶ is each R⁶ is independently hydrogen, halogen, CH(═NH)(NH₂), (═NH), N(R⁷)₂, OR⁷, NHC(O)R⁷, C(O)OR⁷, R⁷-substituted or unsubstituted C₁₋₆alkyl, R⁷-substituted or unsubstituted C₁₋₆haloalkyl, or R⁷-substituted or unsubstituted 4- to 6-membered heterocyclyl; or wherein two or more R⁶ groups together form a C₁₋₂ bridge.

In one embodiment of the compounds described herein, each R⁶ is independently hydrogen, halogen, (═NH), N(R⁷)₂, OR⁷, NHC(O)R⁷, or C(O)OR⁷. In one such embodiment, each R⁶ is independently (═NH), N(R⁷)₂, OR⁷, NHC(O)R⁷, or C(O)OR⁷. In another such embodiment, each R⁶ is independently hydrogen or halogen. In one embodiment, R⁶ is hydrogen.

In another embodiment of the compounds described herein, each R⁶ is independently hydrogen, R⁷-substituted or unsubstituted C₁₋₆alkyl, or R⁷-substituted or unsubstituted C₁₋₆haloalkyl. In one such embodiment, each R⁶ is independently hydrogen or R⁷-substituted or unsubstituted C₁₋₆alkyl. In one such embodiment, each R⁶ is independently hydrogen or R⁷-substituted or unsubstituted C₁₋₃alkyl. In one such embodiment, each R⁶ is independently hydrogen, methyl, or ethyl. In one such embodiment, each R⁶ is independently hydrogen or R⁷-substituted or unsubstituted C₁₋₃haloalkyl. In one such embodiment, each R⁶ is independently hydrogen, CF₃, CHF₂, or CH₂F. In one embodiment of the compounds described herein, R⁶ is independently hydrogen, halogen, OR⁷, R⁷-substituted or unsubstituted C₁₋₆alkyl, or R⁷-substituted or unsubstituted C₁₋₆haloalkyl. In some embodiments, R⁷ is hydrogen, halo, OH, COOH, (═NH), NH₂, unsubstituted C₁₋₃alkyl, or unsubstituted C₁₋₃alkyoxy.

In another embodiment, two or more R⁶ groups together form a C₁₋₂ bridge. In one embodiment, two or more R⁶ groups together form an unsubstituted C₁ bridge. In another embodiment, two or more R⁶ groups together form an unsubstituted C₂ bridge.

In one embodiment of the compounds described herein, R¹ is R⁶-substituted or unsubstituted C₄₋₆ cycloalkyl.

In one embodiment of the compounds described herein, R¹ is R⁶-substituted or unsubstituted 4- to 8-membered heterocyclyl comprising 1 or 2 heteroatoms independently selected from N, O, S, or SO₂. In one embodiment of the compounds described herein, R¹ is R⁶-substituted or unsubstituted 4- to 8-membered heterocyclyl comprising at least one N ring heteroatom. In another embodiment of the compounds described herein, R¹ is R⁶-substituted or unsubstituted 4- to 6-membered heterocyclyl comprising at least 1 N ring heteroatom. In one such embodiment, R¹ is R⁶-substituted or unsubstituted pyrollidinyl. In another such embodiment, R¹ is R⁶-substituted or unsubstituted piperidinyl. In another such embodiment, R¹ is R⁶-substituted or unsubstituted piperazinyl. In another such embodiment, R¹ is R⁶-substituted or unsubstituted azetidinyl. In one such embodiment, R¹ is unsubstituted azetidinyl. In one embodiment, R¹ is R⁶-substituted or unsubstituted 6-membered cyclic guanidinyl. n one embodiment, R¹ is R⁶-substituted or unsubstituted tetrahydropyrimidinyl. In one embodiment, R¹ is R⁶-substituted or unsubstituted morpholino.

In one embodiment of the compounds described herein, R¹ is a moiety of formula:

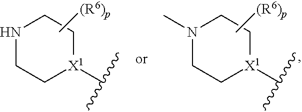

or a stereoisomer thereof, wherein X¹ is N or CH. In one such embodiment, R⁶ is halo. In one such embodiment, R⁶ is F.

In one embodiment of the compounds described herein, R¹ is a moiety of formula:

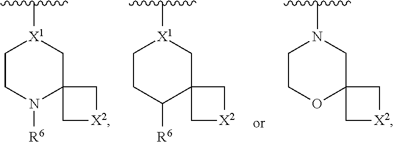

or a stereoisomer thereof, wherein
X² is NR⁶, O or CH; and
X¹ and R⁶ are as described herein. In one such embodiment, X² is N. In another such embodiment, X² is NH or N(CH₃).

In one embodiment of the compounds described herein, R¹ is R⁶-substituted or unsubstituted piperazinyl. In one such embodiment, R¹ is a moiety of formula:

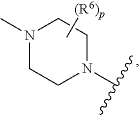

or a stereoisomer thereof, wherein R⁶ is oxo or halo (F), and p is 1 or 2.

In one such embodiment, R¹ is a moiety of formula:

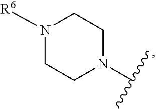

or a stereoisomer thereof. In another such embodiment, R⁶ is —CH₂CH₂OH or —CH₂CH₂CH₂OH. In one embodiment,

In one such embodiment, R¹ is unsubstituted piperazinyl. In one such embodiment, R¹ is a moiety of formula:

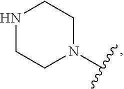 [SMILES: *N1CCNCC1]

or a stereoisomer thereof.

In another embodiment of the compounds described herein, R¹ is R⁶-substituted or unsubstituted azetidinyl, R⁶-substituted or unsubstituted pyrrolidinyl, or R⁶-substituted or unsubstituted piperidinyl. In one such embodiment, R¹ is R⁶-substituted or unsubstituted piperidinyl.

In one such embodiment, R¹ is a moiety of formula

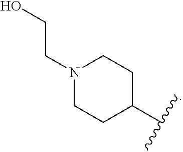 [SMILES: *C1CCN(CCO)CC1]

In another such embodiment, R¹ is a moiety of formula

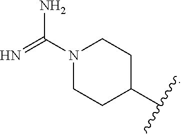 [SMILES: *C1CCN(C(=N)N)CC1]

In one embodiment of the compounds described herein, R¹ is a moiety of formula:

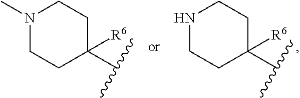

or a stereoisomer thereof

In another embodiment of the compounds described herein, R¹ is a moiety of formula:

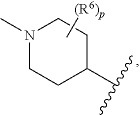

or a stereoisomer thereof, wherein R⁶ is oxo or halo (F), and p is 1 or 2.

In another embodiment of the compounds described herein, R¹ is a moiety of formula:

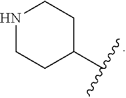 [SMILES: *C1CCNCC1]

In another embodiment of the compounds described herein, R¹ is a moiety of formula:

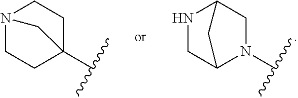 [SMILES: *C12CCN(CC1)C2 ; *N1CC2CC1CN2]

In another embodiment of the compounds described herein, R¹ is a moiety of formula:

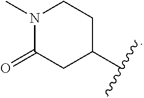 [SMILES: *C1CCN(C)C(=O)C1]

In another embodiment of the compounds described herein, R¹ is a moiety of formula:

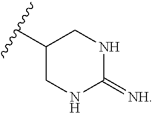 [SMILES: *C1CNC(=N)NC1]

In another embodiment of the compounds described herein, R¹ is a moiety of formula:

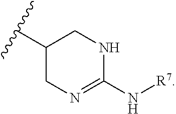

In one such embodiment, R⁷ is tetrahydropyranyl, oxetanyl, CH₂-tetrahydropyranyl, or CH₂-oxetanyl.

In one embodiment of the compounds described herein, R¹ is a moiety of formula:

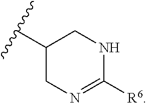

In one such embodiment, R⁶ is a 4-6 membered heterocyclic ring. In one such embodiment, R⁶ is R⁷-substituted or unsubstituted azetidinyl or pyrrolidinyl. In another embodiment, R⁶ is N(R⁷)₂, where each R⁷ is independently hydrogen or unsubstituted C₁₋₃alkyl. In one embodiment, R⁶ is NH((CH₂)₂OH), N((CH₂)₂OH)₂ or NCH((CH₂)₂OH)₂.

In another embodiment of the compounds described herein, R¹ is a moiety of formula:

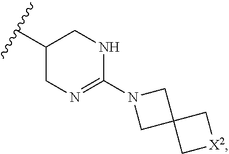

wherein X² is NH, N(CH₃), SO₂, CR⁶, or O. In one such embodiment, X² is O.

In another embodiment of the compounds described herein, R¹ is a moiety of formula

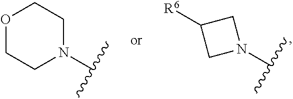

or a stereoisomer thereof. In one such embodiment, R⁶ is azetidinyl or O-azetidinyl.

In another embodiment of the compounds described herein, R¹ is a moiety of formula

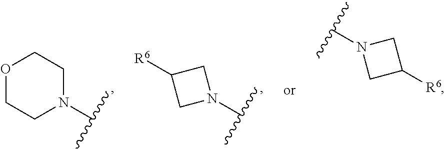

or a stereoisomer thereof.

In another embodiment of the compounds described herein, R¹ is a moiety of formula

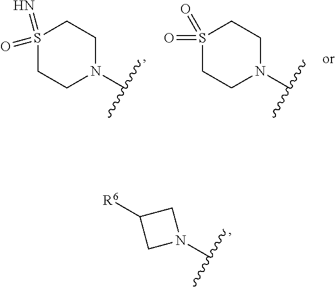

or a stereoisomer thereof.

In another embodiment of the compounds described herein, R¹ is a moiety of formula:

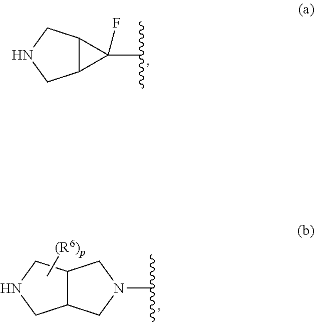

or a stereoisomer thereof. In one such embodiment, the moiety is of formula (a), p is 0. In one such embodiment, the moiety is of formula (a), p is 1 and R⁶ is halo. In another such embodiment, R¹ is a moiety of formula:

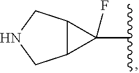 [SMILES: *C1(F)C2CNCC21]

or a stereoisomer thereof.

In one such embodiment, R¹ is a moiety of formula (a) having formula:

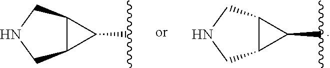 [SMILES: *[C@@H]1[C@H]2CNC[C@@H]12 ; *[C@@H]1[C@H]2CNC[C@@H]12]

In another embodiment of the compounds described herein, R¹ is a moiety of formula:

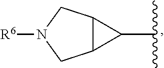

or a stereoisomer thereof. In one such embodiment, R⁶ is CH₃. In one such embodiment, R¹ is a moiety of formula

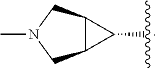 [SMILES: *[C@@H]1[C@H]2CN(C)C[C@@H]12]

In another such embodiment, R⁶ is —CH₂CH₂OH or —CH₂CH₂CH₂OH.

In one embodiment of the compounds described herein, R¹ is a moiety of formula (b) having formula:

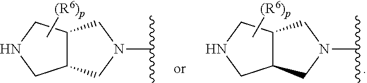

In another embodiment of the compounds described herein, R¹ is a moiety of formula:

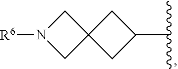

or a stereoisomer thereof. In one such embodiment, R⁶ is hydrogen. In another such embodiment, R⁶ is —CH₂CH₂OH or —CH₂CH₂CH₂OH.

In another embodiment of the compounds described herein, R¹ is a moiety of formula:

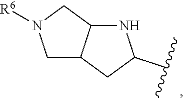

or a stereoisomer thereof, wherein R⁶ is hydrogen. In one such embodiment, R¹ is a moiety of formula

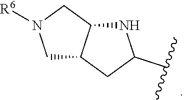

In another embodiment of the compounds described herein, R¹ is —CH₂NH₂ or —CH₂CH₂NH₂.

Further provided herein are compounds of formula (Iz)

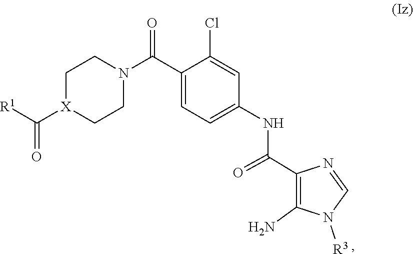

or a stereoisomer or pharmaceutically acceptable salt thereof, where
R¹ is a moiety of formula:

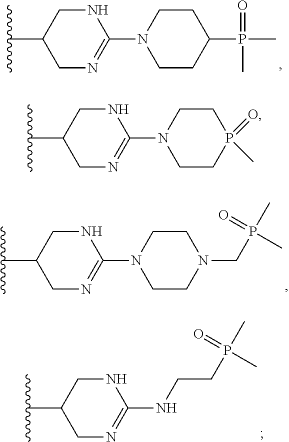

X is CH or N;
and R³ is as described herein.

In one embodiment, the compound comprises a compound of formula (II). In one embodiment of the compounds of formula (II) as described herein, R^{1a} is a moiety of formula:

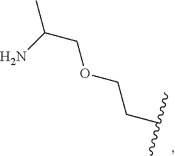 [SMILES: *CCOCC(C)N]

or a stereoisomer thereof.

In one embodiment of the compounds of formula (II) as described herein, R^{1a} is a moiety of formula:

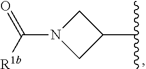

or a stereoisomer thereof. In one embodiment, R^{1b} is unsubstituted 5- to 7-membered heterocyclyl.

In one embodiment of the compounds of formula (II) as described herein, R^{1a} is a moiety of formula:

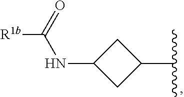

or a stereoisomer thereof.

In one embodiment, R^{1b} is unsubstituted 5- to 7-membered heterocyclyl.

In one embodiment, R^{1b} is O—C₁₋₃alkyaryl.

In one embodiment, R^{1b} is unsubstituted 5- to 7-membered heterocyclyl.

In one embodiment of the compounds of formula (II) described herein, R^{1b} is a moiety of formula

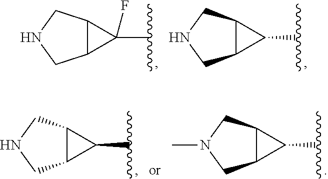

In another such embodiment, R^{1b} is a moiety of formula:

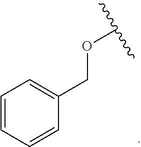 [SMILES: *OCc1ccccc1]

In another such embodiment, R^{1b} is a moiety of formula:

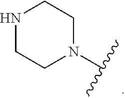 [SMILES: *N1CCNCC1]

In one embodiment of the compounds described herein, R² is hydrogen. In one embodiment of the compounds described herein, R² is unsubstituted C₁₋₃alkyl. In one such embodiment, R⁴ is CH₃. In one embodiment of the compounds described herein, R² is halo. In one such embodiment, R² is F or Cl. In one such embodiment, R² is C₁. In one such embodiment, R² is F. In one embodiment of the compounds described herein, R² is unsubstituted C₁₋₃haloalkyl. In one such embodiment, R² is CH₂F, CHF₂, or CF₃.

In one embodiment of the compounds described herein, R³ is a moiety of formula:

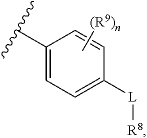

or a stereoisomer thereof.

In one such embodiment, L is —O—. In another embodiment, L is —NH—. In another embodiment, L is —NR^{8b}— wherein R^{8b} is CH₃. In another embodiment, L is —NR^{8b}—, R^{8b} is CH₃; and R⁸ is CH₃. In one embodiment, L is —NH— and R⁸ is unsubstituted cyclopropanol or —CH₂CH₂F. In another embodiment, R⁸ is R^{8a}— substituted or unsubstituted C₃₋₆cycloalkyl and L is absent.

In one such embodiment, R³ is a moiety of formula:

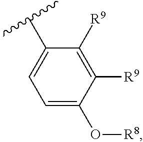

or a stereoisomer thereof, wherein R⁸ and R⁹ are as described herein.

In certain embodiments of the compounds described herein described herein, R⁸ is R^{8a}-substituted or unsubstituted C₁₋₆alkyl, R^{8a}-substituted or unsubstituted C₁₋₃haloalkyl, or R^{8a}-substituted or unsubstituted C₃₋₆cycloalkyl. In one such embodiment, R⁸ is R^{8a}-substituted or unsubstituted C₁₋₆alkyl. In another such embodiment, R⁸ is R^{8a} R^{8a}-substituted or unsubstituted C₁₋₃alkyl. In such embodiments, R⁸ is R⁸ is unsubstituted C₁₋₃alkyl. In one such embodiment, R⁸ is methyl or ethyl.

In another embodiment of the compounds described herein, R⁸ is R⁸ is R^{8a}-substituted or unsubstituted C₁₋₃haloalkyl. In one such embodiment, R⁸ is unsubstituted C₁₋₃haloalkyl. In one embodiment, R⁸ is CF₃, CHF₂, or CH₂F. In another embodiment, R⁸ is CH₂F, CHF₂, CH₃, unsubstituted cyclopropyl.

In one embodiment of the compounds described herein, R⁸ is R^{8a}-substituted or unsubstituted C₃₋₆cycloalkyl. In one such embodiment, R⁸ is R^{8a}-substituted or unsubstituted cyclopropyl. In one such embodiment, R⁸ is unsubstituted cyclopropyl.

In such embodiments, R^{8a} is unsubstituted C₁₋₃alkyl. In one such embodiment, R^{8a} is methyl or ethyl. In another such embodiment, R^{8a} is methyl. In other such embodiments, R^{8a} is OH, or OR^{8b} wherein R^{8b} is unsubstituted C₁₋₃alkyl. In one such embodiment, R^{8a} is OH. In another such embodiment, R^{8a} is OCH₃.

In another such embodiment, R³ is a moiety of formula:

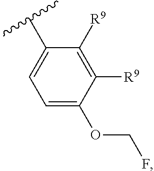

or a stereoisomer thereof, where R⁹ is as described herein. In one such embodiment, each R⁹ of the moiety is halo. In one such embodiment, each R⁹ of the moiety is F.

In one embodiment of the compounds described herein, R⁹ is unsubstituted C₁₋₃alkyl. In one such embodiment, R⁹ is methyl. In another such embodiment, R⁹ is halo. In one such embodiment, R⁹ is F. In another such embodiment, R⁹ is unsubstituted C₁₋₃alkoxy. In another such embodiment, R⁹ is unsubstituted C₁₋₃haloalkyl. In one such embodiment, R⁹ is independently CF₃, CH₂F, or CHF₂. In one embodiment, R⁹ is unsubstituted cyclopropyl. In another such embodiment, R⁹ is CN. In another such embodiment, R⁹ is independently halo, CH₃, CF₃, CH₂F, or CHF₂.

In one embodiment of the compounds described herein, R⁹ is a moiety of formula:

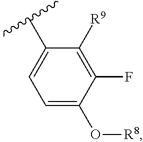

or a stereoisomer thereof, wherein R⁹ is halo or CF₃, CH₂F, or CHF₂ and R⁸ is as described herein. In one such embodiment, R⁸ is CH₂F, CHF₂, unsubstituted cyclopropanol,

In another embodiment of the compounds described herein, R³ is a moiety of formula:

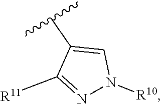

or a stereoisomer thereof.

In one embodiment of the compounds described herein, R¹⁰ is R^{10A}-substituted or unsubstituted C₁₋₆alkyl, R^{10A}-substituted or unsubstituted C₂₋₆alkenyl, or R^{10A}-substituted or unsubstituted C₂₋₆alkynl. In one such embodiment, R¹⁰ is R^{10A}-substituted or unsubstituted C₁₋₃alkyl. In one such embodiment, R¹⁰ is methyl or ethyl. In another such embodiment, R¹⁰ is R^{10A}-substituted or unsubstituted C₂₋₄alkenyl. In one such embodiment, R¹⁰ is unsubstituted C₂₋₄alkenyl. In another such embodiment, R¹⁰ is R^{10A}-substituted or unsubstituted C₂₋₆alkenyl, wherein each R^{10A} is independently halo, CN, OH, OCH₃, or unsubstituted C₃₋₅cycloalkyl. In one such embodiment, R¹⁰ is R^{10A}-substituted or unsubstituted C₂₋₄alkynl. In another embodiment of the compounds described herein, R¹⁰ is unsubstituted cyclobutyl or unsubstituted cyclopropyl.

In one embodiment of the compounds described herein, R^{10A} is halo, CN, OH, OMe, or R^{10B}-substituted or unsubstituted C₃₋₅cycloalkyl, or wherein 2 R^{10A} groups together form a R^{10B}-substituted or unsubstituted C₃₋₄spirocycle. In one embodiment, R^{10A} is halo, CN, OH, OMe, or R^{10B}-substituted or unsubstituted C₃₋₅cycloalkyl. In another such embodiment, each R^{10A} is independently halo. In such embodiments, R^{10A} is F or Cl. In one such embodiment, R^{10A} is F. In another embodiment, R^{10A} is halo, CN, OH, or OMe. In another embodiment of the compounds described herein, R^{10A} is R^{10B}-substituted or unsubstituted C₃₋₅cycloalkyl. In such embodiments, R^{10B} is halo. In one such embodiment, R^{10B} is F or Cl. In such embodiments, R^{10B} is unsubstituted C₁₋₃haloalkyl. In one such embodiment, R^{10B} is CF₃.

In one embodiment of the compounds described herein, R¹¹ is unsubstituted C₁₋₃haloalkyl. In one such embodiment, R¹¹ is CF₃. In another such embodiment, R¹¹ is halo. In one such embodiment, R¹¹ is F or Cl.

In one such embodiment, R³ is a moiety of formula:

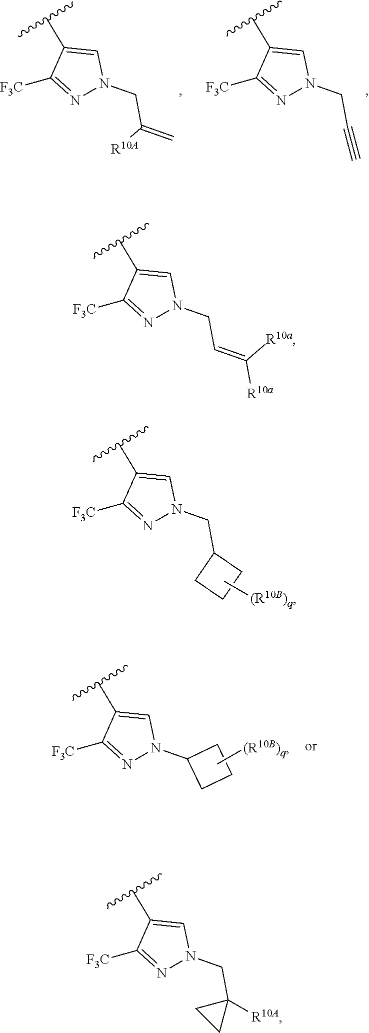

or a stereoisomer thereof. In such embodiments, q is an integer of 0, 1, or 2. In one such embodiment, q is 1. In another such embodiment, q is 2. In another such embodiment, q is 0.

In one embodiment of the compounds described herein, R³ is a moiety of formula:

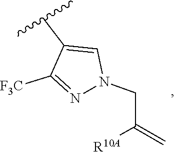

wherein R^{10A} is H, halo, or methyl. In one such embodiment, R^{10A} is F. In another such embodiment, R^{10A} is Cl.

In another embodiment, R³ is a moiety of formula:

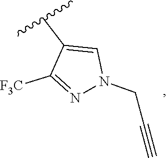 [SMILES: *c1cn(CC#C)nc1C(F)(F)F]

or a stereoisomer thereof.

In another embodiment of the compounds described herein, R³ is a moiety of formula:

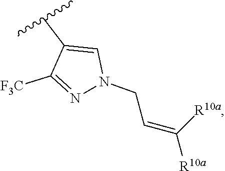

or a stereoisomer thereof, wherein each R^{10A} is halo. In one such embodiment, R^{10A} is F. In another such embodiment, one R^{10A} is halo and one R^{10A} is methyl.

In another embodiment of the compounds described herein, R³ is a moiety of formula:

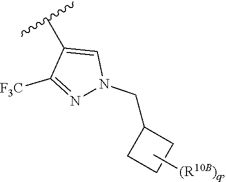

or a stereoisomer thereof, wherein q is 0.

In another embodiment of the compounds described herein, R³ is a moiety of formula:

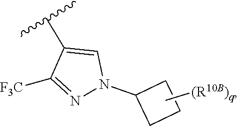

or a stereoisomer thereof wherein q is 0. In another such embodiment, q is 1 and R^{10B} is halo. In one such embodiment, q is 1 and R^{10B} is F.

In another embodiment of the compounds described herein, R³ is a moiety of formula:

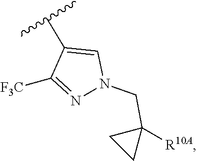

or a stereoisomer thereof, wherein R^{10A} is H, halo, or methyl. In one such embodiment, R^{10A} is F.

In one embodiment of the compounds described herein, R⁴ is hydrogen. In one embodiment of the compounds described herein, R⁴ is halo. In one such embodiment, R⁴ is Cl. In another such embodiment, R⁴ is F. In one embodiment of the compounds described herein, R⁴ is unsubstituted C₁₋₃alkyl. In one such embodiment, R⁴ is CH₃. In another embodiment, R⁹ is hydrogen or halo. In another such embodiment, R⁴ is hydrogen or CH₃.

In one embodiment of the compounds described herein, R³ is a moiety of formula:

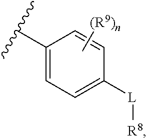

or a stereoisomer thereof and R⁹ is hydrogen or halo.

In another such embodiment, R³ is a moiety of formula:

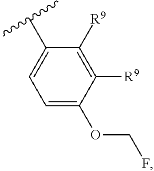

or a stereoisomer thereof and each R⁹ is independently halo, CH₃, CF₃, CH₂F, or CHF₂. In one such embodiment, each R⁹ is F.

In one embodiment of the compounds described herein, the compound has formula:

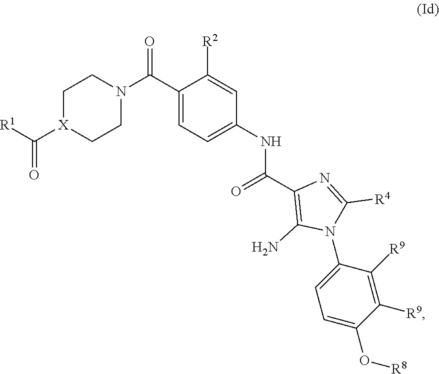

where X, R¹, R², R⁴, R⁸, and R⁹ are as described herein for the compound of formula (I). In one embodiment of the compound of formula (Id), X is N. In another such embodiment, X is CR^{x}. In one such embodiment, X is CH. In one embodiment of the compound of formula (Id), each R⁹ is halo. In one such embodiment, each R⁹ is F. In another such embodiment, each R⁹ is independently halo, CH₃, CF₃, CH₂F, or CHF₂. In one such embodiment, one R⁹ is F and one R⁹ is independently CF₃, CH₂F, or CHF₂. In such embodiments, R⁸ is R⁸ is unsubstituted haloalkyl. In one such embodiment, R⁸ is CH₂F, CHF₂, CH₃, unsubstituted cyclopropyl. In one embodiment, R⁸ is CH₂F. In another such embodiment, R⁸ is unsubstituted cyclopropyl. In such embodiments, R⁴ is hydrogen. In other such embodiments, R² is R² is halo.

In embodiments of the compound of formula (Id), R¹ is a moiety of formula:

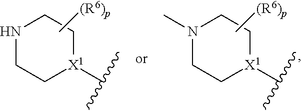

or a stereoisomer thereof, wherein X¹ is N or CH. In one such embodiment, R⁶ is halo. In one such embodiment, R⁶ is F. In another embodiment, p is 0.

In one such embodiment, R¹ is a moiety of formula:

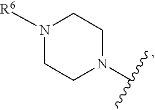

or a stereoisomer thereof. In one such embodiment, R⁶ is —CH₂CH₂OH or —CH₂CH₂CH₂OH.

In one such embodiment, R¹ is a moiety of formula:

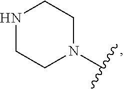 [SMILES: *N1CCNCC1]

or a stereoisomer thereof.

In another embodiment, R¹ is a moiety of formula:

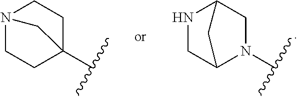 [SMILES: *C12CCN(CC1)C2 ; *N1CC2CC1CN2]

In another embodiment, R¹ is a moiety of formula:

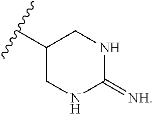 [SMILES: *C1CNC(=N)NC1]

In another embodiment, R¹ is a moiety of formula:

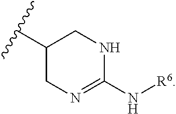

In another embodiment of the compounds described herein the compound has formula:

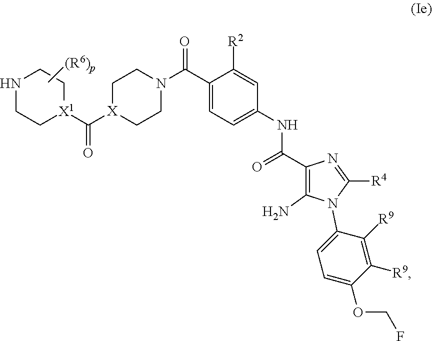

where X¹, p, R², R⁴, R⁶, and R⁹ are as described herein for the compound of formula (I). In one embodiment of the compound of formula (Ie), R² is halo and R⁴ is hydrogen. In one embodiment of the compound of formula (Ie), each R⁹ is halo. In one such embodiment, each R⁹ is F. In another such embodiment, each R⁹ is independently halo, CH₃, CF₃, CH₂F, or CHF₂. In one such embodiment, one R⁹ is F and one R⁹ is independently CF₃, CH₂F, or CHF₂. In one embodiment of the compound of formula (Ie), X is N and X¹ is CH. In another embodiment, X is CH and X¹ is CH. In one embodiment of the compound of formula (Ie), X is N or CH; X¹ is CH; R² is halo; and R⁴ is hydrogen.

In one embodiment of the compounds described herein, the compound is a compound as set forth in Table 1. In one embodiment, the compound is a compound having the formula of Compound number 271, 204, 203, 197, 159, 29, 43, 91, 76, 135, 31, 77, 83, 88, or 105. In one embodiment, the compound is a compound having the formula of Compound number 204, 203, 197, 159, 29, 31, 77, 83, 88, or 105. In another embodiment, the compound is a compound having the formula of Compound number 204, 203, 197, 31, 77, 83, or 88. In another embodiment, the compound is a compound having formula of Compound number 159, 29, or 105. In another embodiment, the compound is a compound having the formula of Compound number 83, 88, 204, or 31.

Further provided herein are compounds of formula (III):

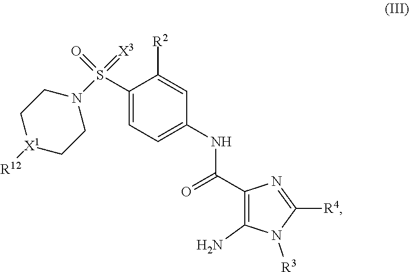

or a stereoisomer or pharmaceutically acceptable salt thereof, wherein
X³ is O or NH;
R¹² is hydrogen, unsubstituted C₁₋₃alkyl, or C(O)R¹;
and X¹, R¹, R², R³, and R⁴ are as defined herein for compounds of formula (I).

TABLE 1

| Cmpd | | |
|---|---|---|
| No | Structure | Chemical Name |
| 1 | 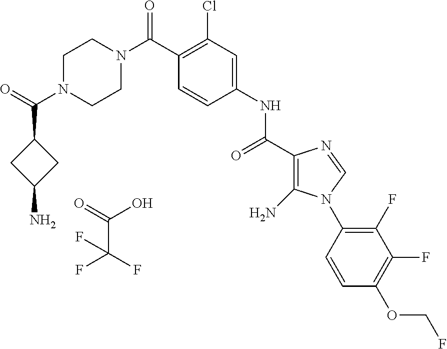 | 5-amino-N-[4-[4-((1s,3s)-3- aminocyclobutanecarbonyl)piperazine- 1-carbonyl]-3-chloro-phenyl]-1-[2,3- difluoro-4- (fluoromethoxy)phenyl]imidazole-4- carboxamide. 1:1.2 2,2,2-trifluoroacetic acid |
| 2 | 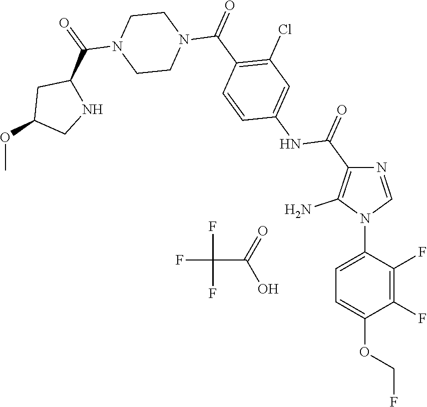 [SMILES: CO[C@@H]1CN[C@H](C(=O)N2CCN(C(=O)c3ccc(NC(=O)c4ncn(-c5ccc(OCF)c(F)c5F)c4N)cc3Cl)CC2)C1 ; O=C(O)C(F)(F)F] | 5-amino-N-[3-chloro-4-[4-[(2S,4S)-4- methoxyprolyl]piperazine-1- carbonyl]phenyl]-1-[2,3-difluoro-4- (fluoromethoxy)phenyl]imidazole-4- carboxamide. 1:1 2,2,2-trifluoroacetic acid |
| 3 | 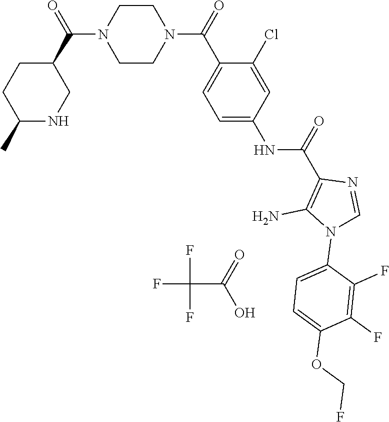 [SMILES: C[C@H]1CC[C@@H](C(=O)N2CCN(C(=O)c3ccc(NC(=O)c4ncn(-c5ccc(OCF)c(F)c5F)c4N)cc3Cl)CC2)CN1 ; O=C(O)C(F)(F)F] | 5-amino-N-[3-chloro-4-[4-[(3R,6S)-6- methylnipecotoyl]piperazine-1- carbonyl]phenyl]-1-[2,3-difluoro-4- (fluoromethoxy)phenyl]imidazole-4- carboxamide. 1:2 2,2,2-trifluoroacetic acid |
| 4 | 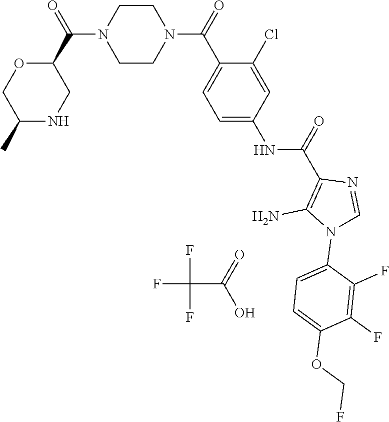 [SMILES: C[C@H]1CO[C@@H](C(=O)N2CCN(C(=O)c3ccc(NC(=O)c4ncn(-c5ccc(OCF)c(F)c5F)c4N)cc3Cl)CC2)CN1 ; O=C(O)C(F)(F)F] | 5-amino-N-[3-chloro-4-[4-[(2R,5S)-5- methylmorpholine-2- carbonyl]piperazine-1-carbonyl]phenyl]- 1-[2,3-difluoro-4- (fluoromethoxy)phenyl]imidazole-4- carboxamide. 1:2 2,2,2-trifluoroacetic acid |
| 5 | 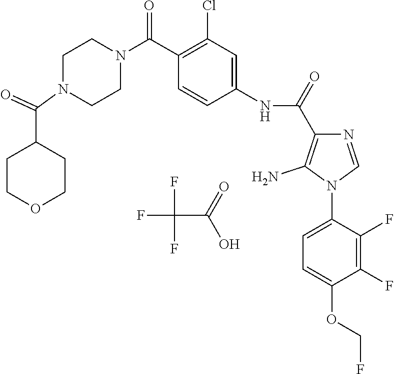 [SMILES: Nc1c(C(=O)Nc2ccc(C(=O)N3CCN(C(=O)C4CCOCC4)CC3)c(Cl)c2)ncn1-c1ccc(OCF)c(F)c1F ; O=C(O)C(F)(F)F] | 5-amino-N-[3-chloro-4-[4- (tetrahydropyran-4-carbonyl)piperazine- 1-carbonyl]phenyl]-1-[2,3-difluoro-4- (fluoromethoxy)phenyl]imidazole-4- carboxamide. 1:1 2,2,2-trifluoroacetic acid |
| 6 | 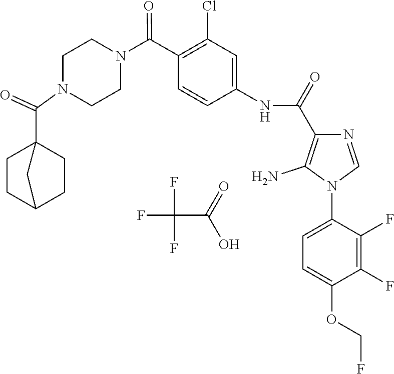 [SMILES: Nc1c(C(=O)Nc2ccc(C(=O)N3CCN(C(=O)C45CCC(CC4)C5)CC3)c(Cl)c2)ncn1-c1ccc(OCF)c(F)c1F ; O=C(O)C(F)(F)F] | 5-amino-N-[4-[4-(1- azabicyclo[2.2.1]heptane-4- carbonyl)piperazine-1-carbonyl]-3- chloro-phenyl]-1-[2,3-difluoro-4- (fluoromethoxy)phenyl]imidazole-4- carboxamide. 1:1 2,2,2-trifluoroacetic acid |
| 7 | 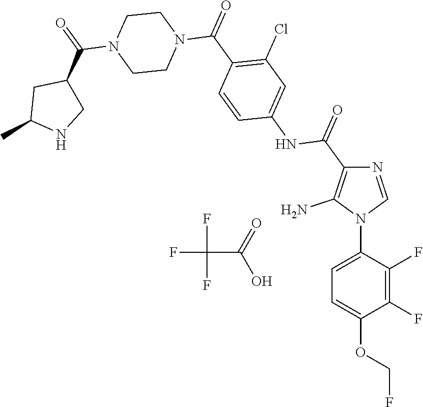 [SMILES: C[C@H]1C[C@@H](C(=O)N2CCN(C(=O)c3ccc(NC(=O)c4ncn(-c5ccc(OCF)c(F)c5F)c4N)cc3Cl)CC2)CN1 ; O=C(O)C(F)(F)F] | 5-amino-N-[3-chloro-4-[4-[(3R,5S)-5- methylpyrrolidine-3-carbonyl]piperazine- 1-carbonyl]phenyl]-1-[2,3-difluoro-4- (fluoromethoxy)phenyl]imidazole-4- carboxamide. 1:1 2,2,2-trifluoroacetic acid |
| 8 | 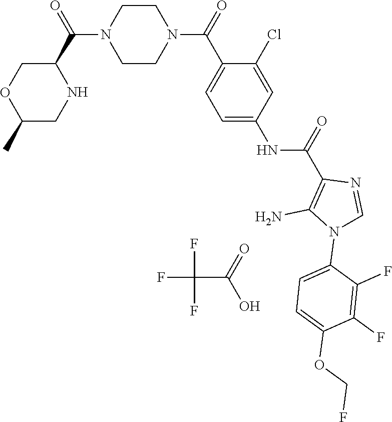 | 5-amino-N-[3-chloro-4-[4-[(3S,6R)-6- methylmorpholine-3- carbonyl]piperazine-1-carbonyl]phenyl]- 1-[2,3-difluoro-4- (fluoromethoxy)phenyl]imidazole-4- carboxamide. 1:1 2,2,2-trifluoroacetic acid |
| 9 | 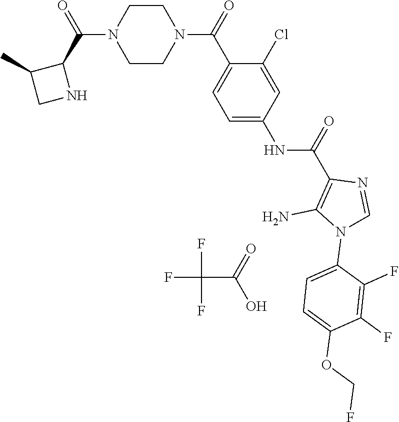 | 5-amino-N-[3-chloro-4-[4-[(2S,3R)-3- methylazetidine-2-carbonyl]piperazine- 1-carbonyl]phenyl]-1-[2,3-difluoro-4- (fluoromethoxy)phenyl]imidazole-4- carboxamide. 1:2 2,2,2-trifluoroacetic acid |
| 10 | 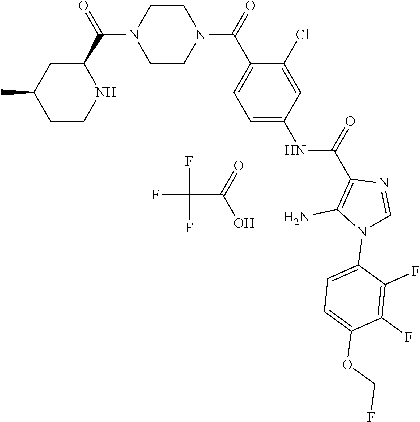 | 5-amino-N-[3-chloro-4-[4-[(2S,4R)-4- methylpipecoloyl]piperazine-1- carbonyl]phenyl]-1-[2,3-difluoro-4- (fluoromethoxy)phenyl]imidazole-4- carboxamide. 1:1.2 2,2,2-trifluoroacetic acid |
| 11 | 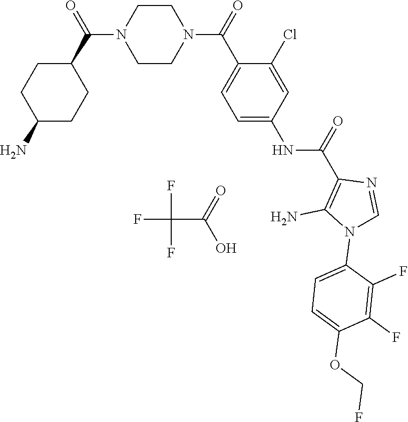 [SMILES: Nc1c(C(=O)Nc2ccc(C(=O)N3CCN(C(=O)[C@H]4CC[C@@H](N)CC4)CC3)c(Cl)c2)ncn1-c1ccc(OCF)c(F)c1F ; O=C(O)C(F)(F)F] | 5-amino-N-[4-[4-((1s,4s)-4- aminocyclohexanecarbonyl)piperazine- 1-carbonyl]-3-chloro-phenyl]-1-[2,3- difluoro-4- (fluoromethoxy)phenyl]imidazole-4- carboxamide. 1:1 2,2,2-trifluoroacetic acid |
| 12 | 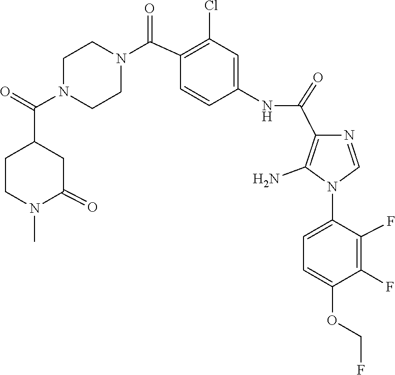 [SMILES: CN1CCC(C(=O)N2CCN(C(=O)c3ccc(NC(=O)c4ncn(-c5ccc(OCF)c(F)c5F)c4N)cc3Cl)CC2)CC1=O] | 5-amino-N-[3-chloro-4-[4-(2-keto-1- methyl-isonipecotoyl)piperazine-1- carbonyl]phenyl]-1-[2,3-difluoro-4- (fluoromethoxy)phenyl]imidazole-4- carboxamide. 1:2 hydrogen chloride |
| 13 | 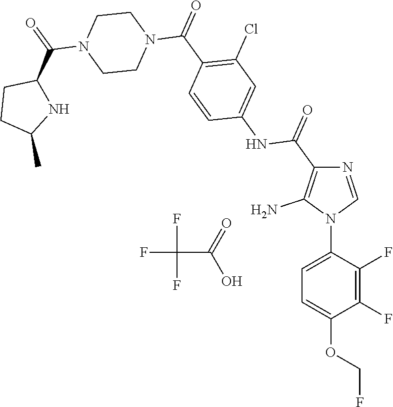 [SMILES: C[C@H]1CC[C@@H](C(=O)N2CCN(C(=O)c3ccc(NC(=O)c4ncn(-c5ccc(OCF)c(F)c5F)c4N)cc3Cl)CC2)N1 ; O=C(O)C(F)(F)F] | 5-amino-N-[3-chloro-4-[4-[(2S,5S)-5- methylprolyl]piperazine-1- carbonyl]phenyl]-1-[2,3-difluoro-4- (fluoromethoxy)phenyl]imidazole-4- carboxamide. 1:1 2,2,2-trifluoroacetic acid |
| 14 | 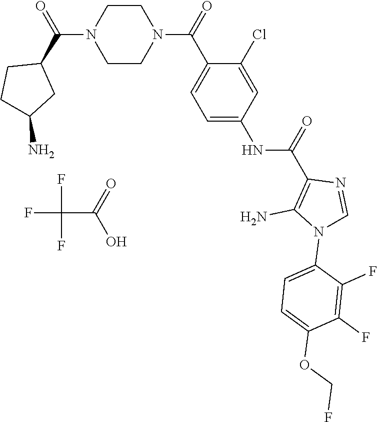 | 5-amino-N-[4-[4-[(1R,3S)-3- aminocyclopentanecarbonyl]piperazine- 1-carbonyl]-3-chloro-phenyl]-1-[2,3- difluoro-4- (fluoromethoxy)phenyl]imidazole-4- carboxamide. 1:1 2,2,2-trifluoroacetic acid |
| 15 | 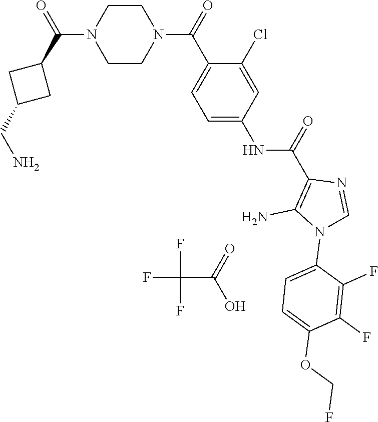 | 5-amino-N-[4-[4-[(1r,3r)-3- (aminomethyl)cyclobutanecarbonyl] piperazine-1-carbonyl]-3-chloro- phenyl]-1-[2,3-difluoro-4- (fluoromethoxy)phenyl]imidazole-4- carboxamide. 1:2 2,2,2-trifluoroacetic acid |
| 16 | 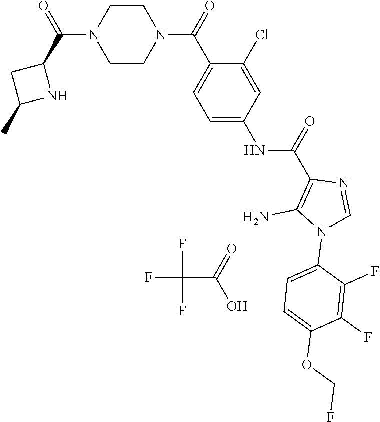 | 5-amino-N-[3-chloro-4-[4-[(2S,4S)-4- methylazetidine-2-carbonyl]piperazine- 1-carbonyl]phenyl]-1-[2,3-difluoro-4- (fluoromethoxy)phenyl]imidazole-4- carboxamide. 1:2 2,2,2-trifluoroacetic acid |
| 17 | 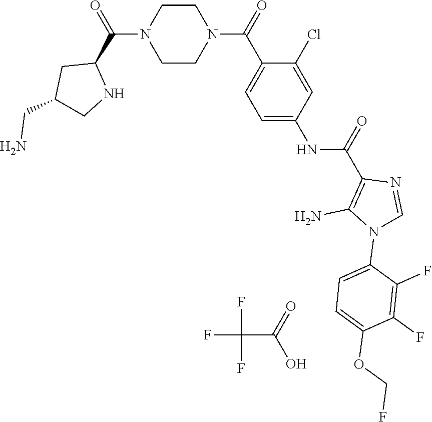 | 5-amino-N-[4-[4-[(2S,4S)-4- (aminomethyl)prolyl]piperazine-1- carbonyl]-3-chloro-phenyl]-1-[2,3- difluoro-4- (fluoromethoxy)phenyl]imidazole-4- carboxamide. 1:2 2,2,2-trifluoroacetic acid |
| 18 | 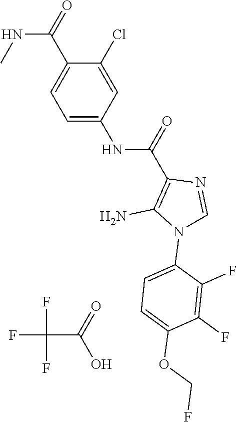 | 5-amino-N-[3-chloro-4- (methylcarbamoyl)phenyl]-1-[2,3- difluoro-4- (fluoromethoxy)phenyl]imidazole-4- carboxamide. 1:1 2,2,2-trifluoroacetic acid |
| 19 | 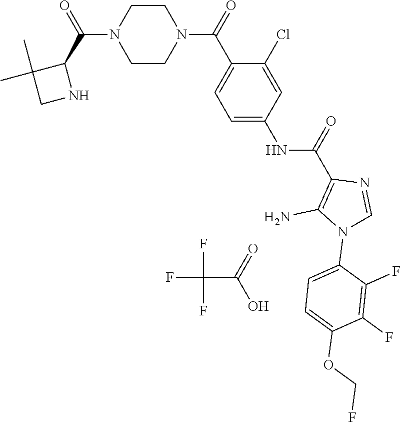 | 5-amino-N-[3-chloro-4-[4-[(2S)-3,3- dimethylazetidine-2- carbonyl]piperazine-1-carbonyl]phenyl]- 1-[2,3-difluoro-4- (fluoromethoxy)phenyl]imidazole-4- carboxamide. 1:2 2,2,2-trifluoroacetic acid |
| 20 | 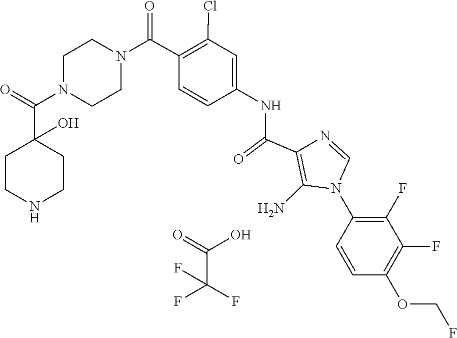 [SMILES: Nc1c(C(=O)Nc2ccc(C(=O)N3CCN(C(=O)C4(O)CCNCC4)CC3)c(Cl)c2)ncn1-c1ccc(OCF)c(F)c1F ; O=C(O)C(F)(F)F] | 5-amino-N-[3-chloro-4-[4-(4- hydroxyisonipecotoyl)piperazine-1- carbonyl]phenyl]-1-[2,3-difluoro-4- (fluoromethoxy)phenyl]imidazole-4- carboxamide. 1:1 2,2,2-trifluoroacetic acid |
| 21 | 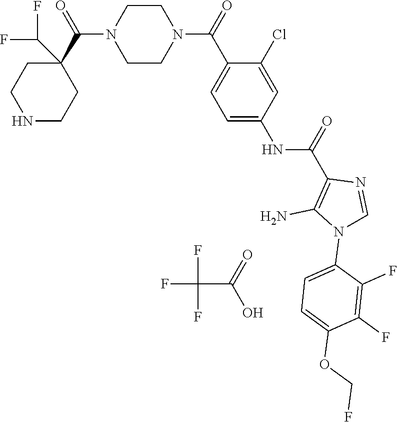 | 5-amino-N-[3-chloro-4-[4-[4- (difluoromethyl)isonipecotoyl]piperazine- 1-carbonyl]phenyl]-1-[2,3-difluoro-4- (fluoromethoxy)phenyl]imidazole-4- carboxamide. 1:2 2,2,2-trifluoroacetic acid |
| 22 | 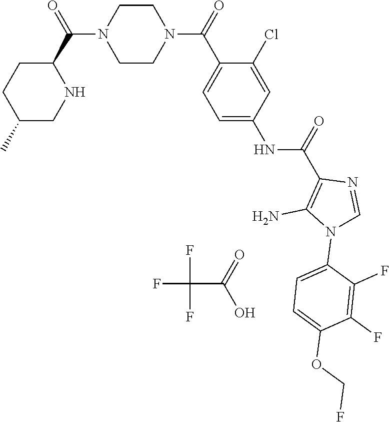 [SMILES: C[C@@H]1CC[C@@H](C(=O)N2CCN(C(=O)c3ccc(NC(=O)c4ncn(-c5ccc(OCF)c(F)c5F)c4N)cc3Cl)CC2)NC1 ; O=C(O)C(F)(F)F] | 5-amino-N-[3-chloro-4-[4-[(2S,5R)-5- methylpipecoloyl]piperazine-1- carbonyl]phenyl]-1-[2,3-difluoro-4- (fluoromethoxy)phenyl]imidazole-4- carboxamide. 1:2 2,2,2-trifluoroacetic acid |
| 23 | 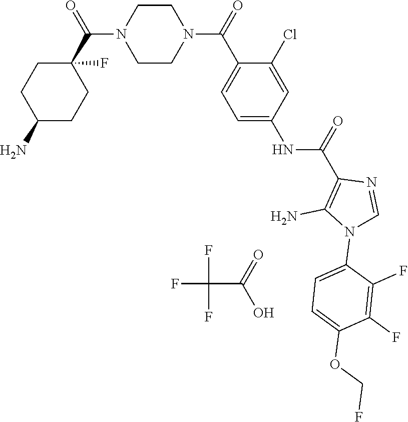 [SMILES: Nc1c(C(=O)Nc2ccc(C(=O)N3CCN(C(=O)[C@]4(F)CC[C@H](N)CC4)CC3)c(Cl)c2)ncn1-c1ccc(OCF)c(F)c1F ; O=C(O)C(F)(F)F] | 5-amino-N-[4-[4-((1r,4r)-4-amino-1- fluoro-cyclohexanecarbonyl)piperazine- 1-carbonyl]-3-chloro-phenyl]-1-[2,3- difluoro-4- (fluoromethoxy)phenyl]imidazole-4- carboxamide. 1:3 2,2,2-trifluoroacetic acid |
| 24 | 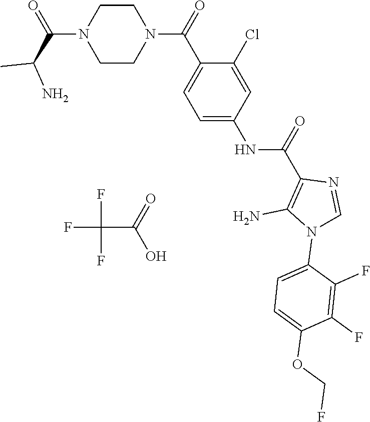 [SMILES: C[C@H](N)C(=O)N1CCN(C(=O)c2ccc(NC(=O)c3ncn(-c4ccc(OCF)c(F)c4F)c3N)cc2Cl)CC1 ; O=C(O)C(F)(F)F] | 5-amino-N-[4-[4-[(2S)-2- aminopropanoyl]piperazine-1-carbonyl]- 3-chloro-phenyl]-1-[2,3-difluoro-4- (fluoromethoxy)phenyl]imidazole-4- carboxamide. 1:1 2,2,2-trifluoroacetic acid |
| 25 | 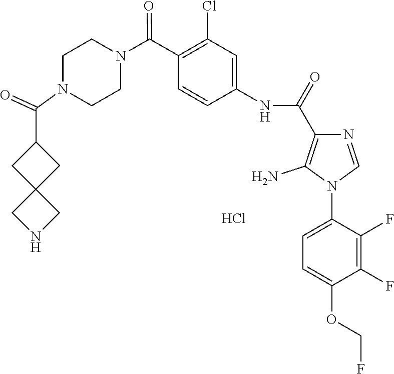 [SMILES: Nc1c(C(=O)Nc2ccc(C(=O)N3CCN(C(=O)C4CC5(CNC5)C4)CC3)c(Cl)c2)ncn1-c1ccc(OCF)c(F)c1F ; Cl] | 5-amino-N-[4-[4-(2- azaspiro[3.3]heptane-6- carbonyl)piperazine-1-carbonyl]-3- chloro-phenyl]-1-[2,3-difluoro-4- (fluoromethoxy)phenyl]imidazole-4- carboxamide. 1:2 hydrogen chloride |
| 26 | 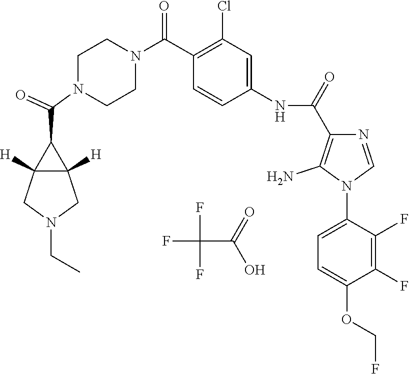 [SMILES: CCN1C[C@@H]2[C@H](C1)[C@H]2C(=O)N1CCN(C(=O)c2ccc(NC(=O)c3ncn(-c4ccc(OCF)c(F)c4F)c3N)cc2Cl)CC1 ; O=C(O)C(F)(F)F] | 5-amino-N-[3-chloro-4-[4-[(1R,5S,6r)-3- ethyl-3-azabicyclo[3.1.0]hexane-6- carbonyl]piperazine-1-carbonyl]phenyl]- 1-[2,3-difluoro-4- (fluoromethoxy)phenyl]imidazole-4- carboxamide. 1:1 2,2,2-trifluoroacetic acid |
| 27 | 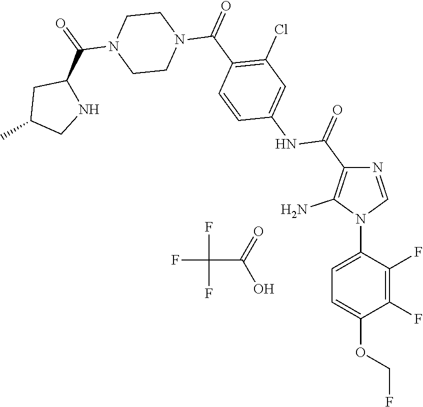 [SMILES: C[C@H]1CN[C@H](C(=O)N2CCN(C(=O)c3ccc(NC(=O)c4ncn(-c5ccc(OCF)c(F)c5F)c4N)cc3Cl)CC2)C1 ; O=C(O)C(F)(F)F] | 5-amino-N-[3-chloro-4-[4-[(2S,4R)-4- methylprolyl]piperazine-1- carbonyl]phenyl]-1-[2,3-difluoro-4- (fluoromethoxy)phenyl]imidazole-4- carboxamide. 1:1 2,2,2-trifluoroacetic acid |
| 28 | 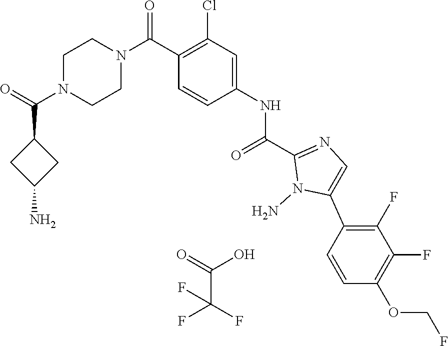 | 5-amino-N-[4-[4-((1r,3r)-3- aminocyclobutanecarbonyl)piperazine- 1-carbonyl]-3-chloro-phenyl]-1-[2,3- difluoro-4- (fluoromethoxy)phenyl]imidazole-4- carboxamide. 1:.93 2,2,2-trifluoroacetic acid |
| 29 | 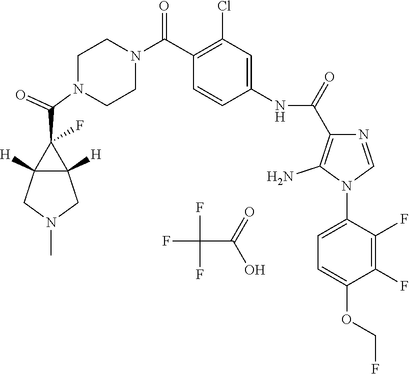 [SMILES: CN1C[C@@H]2[C@H](C1)[C@@]2(F)C(=O)N1CCN(C(=O)c2ccc(NC(=O)c3ncn(-c4ccc(OCF)c(F)c4F)c3N)cc2Cl)CC1 ; O=C(O)C(F)(F)F] | 5-amino-N-[3-chloro-4-[4-[(1R,5S,6r)-3- methyl-3-azabicyclo[3.1.0]hexane-6- carbonyl]piperazine-1-carbonyl]phenyl]- 1-[2,3-difluoro-4- (fluoromethoxy)phenyl]imidazole-4- carboxamide. 1:1 2,2,2-trifluoroacetic acid |
| 30 | 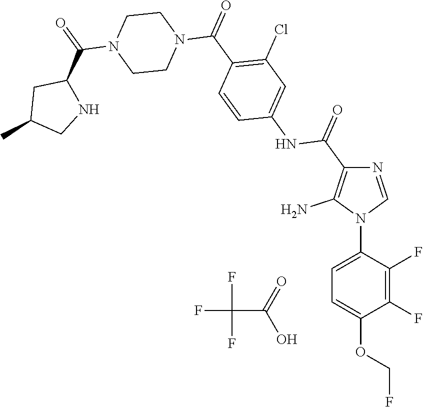 [SMILES: C[C@@H]1CN[C@H](C(=O)N2CCN(C(=O)c3ccc(NC(=O)c4ncn(-c5ccc(OCF)c(F)c5F)c4N)cc3Cl)CC2)C1 ; O=C(O)C(F)(F)F] | 5-amino-N-[3-chloro-4-[4-[(2S,4S)-4- methylprolyl]piperazine-1- carbonyl]phenyl]-1-[2,3-difluoro-4- (fluoromethoxy)phenyl]imidazole-4- carboxamide. 1:1 2,2,2-trifluoroacetic acid |
| 31 | 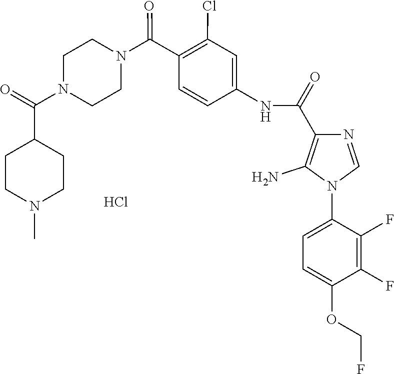 [SMILES: CN1CCC(C(=O)N2CCN(C(=O)c3ccc(NC(=O)c4ncn(-c5ccc(OCF)c(F)c5F)c4N)cc3Cl)CC2)CC1 ; Cl] | 5-amino-N-[3-chloro-4-[4-(1- methylisonipecotoyl)piperazine-1- carbonyl]phenyl]-1-[2,3-difluoro-4- (fluoromethoxy)phenyl]imidazole-4- carboxamide. 1:2 hydrogen chloride |
| 32 | 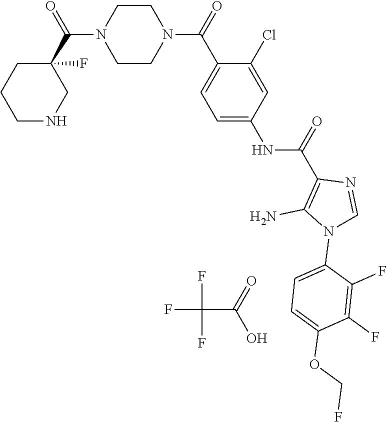 | 5-amino-N-[3-chloro-4-[4-[(3S)-3- fluoronipecotoyl]piperazine-1- carbonyl]phenyl]-1-[2,3-difluoro-4- (fluoromethoxy)phenyl]imidazole-4- carboxamide. 1:2 2,2,2-trifluoroacetic acid |
| 33 | 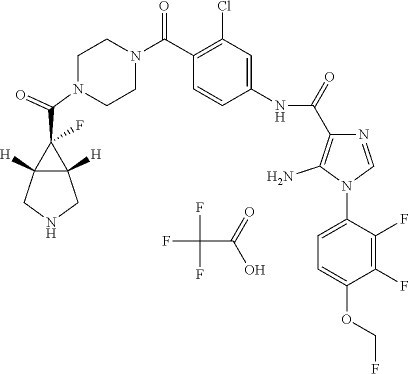 [SMILES: Nc1c(C(=O)Nc2ccc(C(=O)N3CCN(C(=O)[C@@]4(F)[C@@H]5CNC[C@@H]54)CC3)c(Cl)c2)ncn1-c1ccc(OCF)c(F)c1F ; O=C(O)C(F)(F)F] | 5-amino-N-[3-chloro-4-[4-[(1S,5R,6r)-6- fluoro-3-azabicyclo[3.1.0]hexane-6- carbonyl]piperazine-1-carbonyl]phenyl]- 1-[2,3-difluoro-4- (fluoromethoxy)phenyl]imidazole-4- carboxamide. 1:1 2,2,2-trifluoroacetic acid |
| 34 | 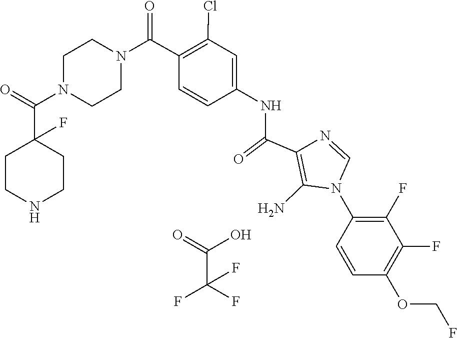 [SMILES: Nc1c(C(=O)Nc2ccc(C(=O)N3CCN(C(=O)C4(F)CCNCC4)CC3)c(Cl)c2)ncn1-c1ccc(OCF)c(F)c1F ; O=C(O)C(F)(F)F] | 5-amino-N-[3-chloro-4-[4-(4- fluoroisonipecotoyl)piperazine-1- carbonyl]phenyl]-1-[2,3-difluoro-4- (fluoromethoxy)phenyl]imidazole-4- carboxamide. 1:1.2 2,2,2-trifluoroacetic acid |
| 35 | 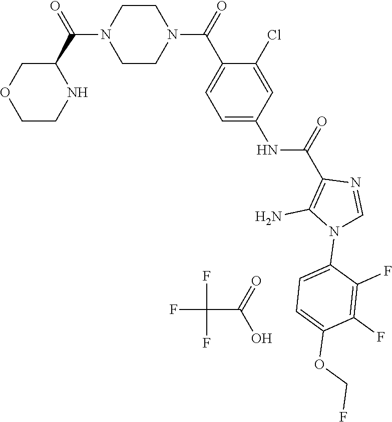 | 5-amino-N-[3-chloro-4-[4-[(3S)- morpholine-3-carbonyl]piperazine-1- carbonyl]phenyl]-1-[2,3-difluoro-4- (fluoromethoxy)phenyl]imidazole-4- carboxamide. 1:2 2,2,2-trifluoroacetic acid |
| 36 | 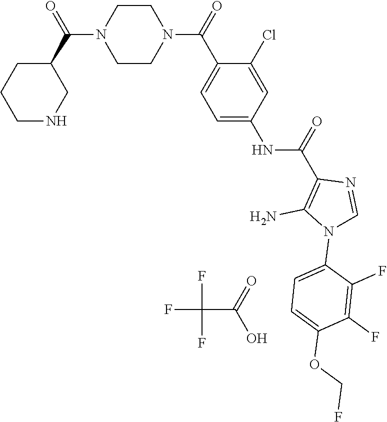 | 5-amino-N-[3-chloro-4-[4-[(2R)- morpholine-2-carbonyl]piperazine-1- carbonyl]phenyl]-1-[2,3-difluoro-4- (fluoromethoxy)phenyl]imidazole-4- carboxamide. 1:2 2,2,2-trifluoroacetic acid |
| 37 | 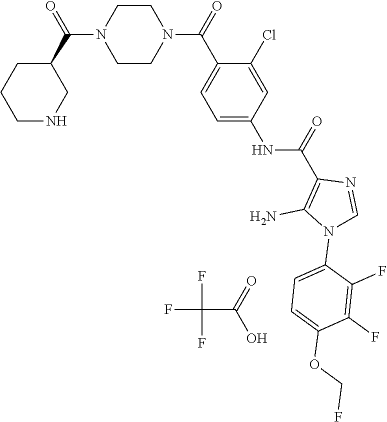 | 5-amino-N-[3-chloro-4-[4-[(3R)- nipecotoyl]piperazine-1- carbonyl]phenyl]-1-[2,3-difluoro-4- (fluoromethoxy)phenyl]imidazole-4- carboxamide. 1:2 2,2,2-trifluoroacetic acid |
| 38 | 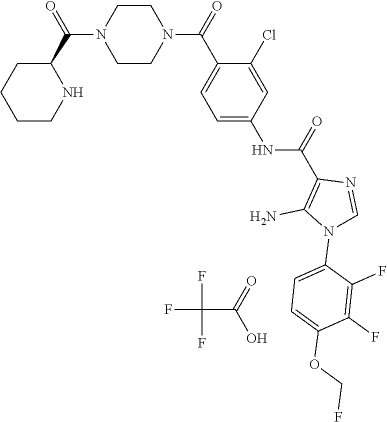 | 5-amino-N-[3-chloro-4-[4-[(2S)- pipecoloyl]piperazine-1- carbonyl]phenyl]-1-[2,3-difluoro-4- (fluoromethoxy)phenyl]imidazole-4- carboxamide. 1:2 2,2,2-trifluoroacetic acid |
| 39 | 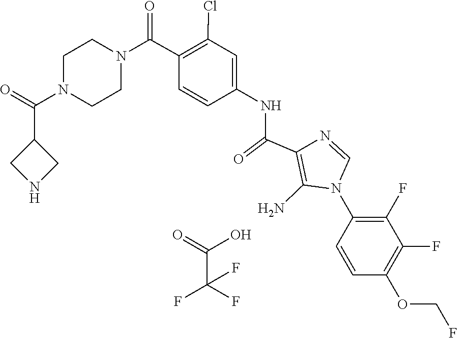 [SMILES: Nc1c(C(=O)Nc2ccc(C(=O)N3CCN(C(=O)C4CNC4)CC3)c(Cl)c2)ncn1-c1ccc(OCF)c(F)c1F ; O=C(O)C(F)(F)F] | 5-amino-N-[4-[4-(azetidine-3- carbonyl)piperazine-1-carbonyl]-3- chloro-phenyl]-1-[2,3-difluoro-4- (fluoromethoxy)phenyl]imidazole-4- carboxamide. 1:1.2 2,2,2-trifluoroacetic acid |
| 40 | 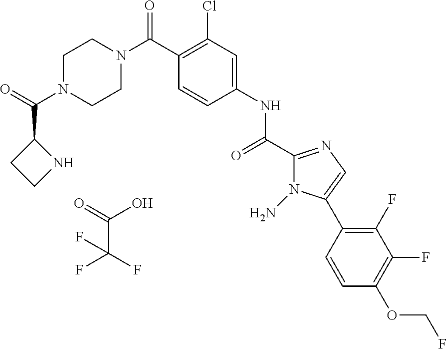 | 5-amino-N-[4-[4-[(2S)-azetidine-2- carbonyl]piperazine-1-carbonyl]-3- chloro-phenyl]-1-[2,3-difluoro-4- (fluoromethoxy)phenyl]imidazole-4- carboxamide. 1:1.1 2,2,2-trifluoroacetic acid |
| 41 | 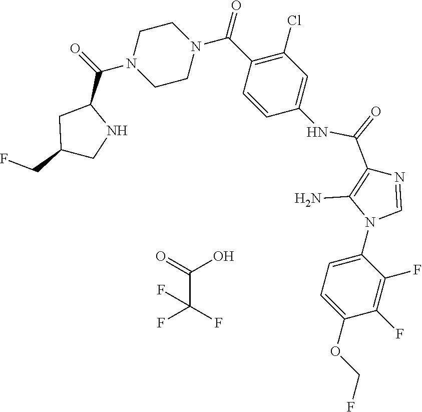 [SMILES: Nc1c(C(=O)Nc2ccc(C(=O)N3CCN(C(=O)[C@@H]4C[C@H](CF)CN4)CC3)c(Cl)c2)ncn1-c1ccc(OCF)c(F)c1F ; O=C(O)C(F)(F)F] | 5-amino-N-[3-chloro-4-[4-[(2S,4S)-4- (fluoromethyl)prolyl]piperazine-1- carbonyl]phenyl]-1-[2,3-difluoro-4- (fluoromethoxy)phenyl]imidazole-4- carboxamide. 1:1.1 2,2,2-trifluoroacetic acid |
| 42 | 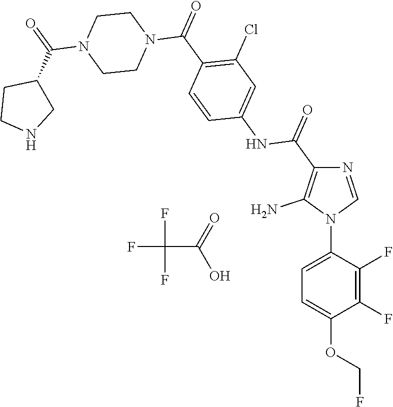 [SMILES: Nc1c(C(=O)Nc2ccc(C(=O)N3CCN(C(=O)[C@H]4CCNC4)CC3)c(Cl)c2)ncn1-c1ccc(OCF)c(F)c1F ; O=C(O)C(F)(F)F] | 5-amino-N-[3-chloro-4-[4-[(3S)- pyrrolidine-3-carbonyl]piperazine-1- carbonyl]phenyl]-1-[2,3-difluoro-4- (fluoromethoxy)phenyl]imidazole-4- carboxamide. 1:1 2,2,2-trifluoroacetic acid |
| 43 | 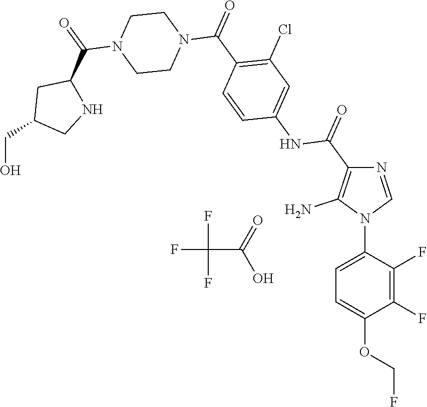 [SMILES: Nc1c(C(=O)Nc2ccc(C(=O)N3CCN(C(=O)[C@@H]4C[C@@H](CO)CN4)CC3)c(Cl)c2)ncn1-c1ccc(OCF)c(F)c1F ; O=C(O)C(F)(F)F] | 5-amino-N-[3-chloro-4-[4-[(2S,4R)-4- methylolprolyl]piperazine-1- carbonyl]phenyl]-1-[2,3-difluoro-4- (fluoromethoxy)phenyl]imidazole-4- carboxamide. 1:1 2,2,2-trifluoroacetic acid |
| 44 | 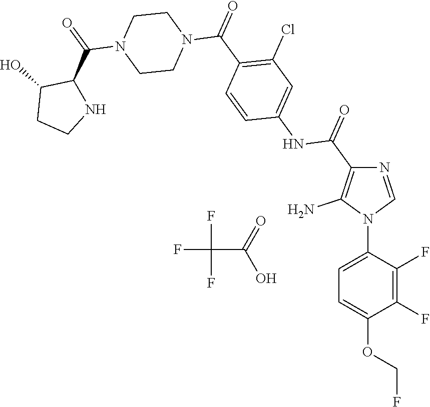 [SMILES: Nc1c(C(=O)Nc2ccc(C(=O)N3CCN(C(=O)[C@H]4NCC[C@@H]4O)CC3)c(Cl)c2)ncn1-c1ccc(OCF)c(F)c1F ; O=C(O)C(F)(F)F] | 5-amino-N-[3-chloro-4-[4-[(2S,3S)-3- hydroxyprolyl]piperazine-1- carbonyl]phenyl]-1-[2,3-difluoro-4- (fluoromethoxy)phenyl]imidazole-4- carboxamide. 1:1 2,2,2-trifluoroacetic acid |
| 45 | 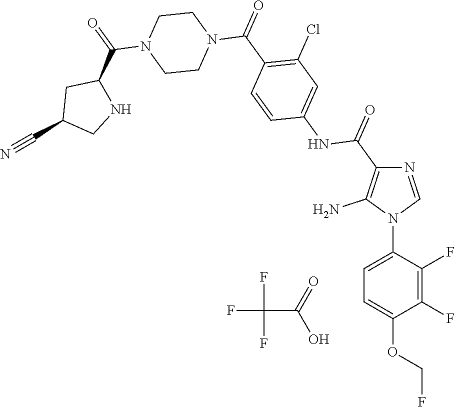 [SMILES: N#C[C@@H]1CN[C@H](C(=O)N2CCN(C(=O)c3ccc(NC(=O)c4ncn(-c5ccc(OCF)c(F)c5F)c4N)cc3Cl)CC2)C1 ; O=C(O)C(F)(F)F] | 5-amino-N-[3-chloro-4-[4-[(2S,4S)-4- cyanoprolyl]piperazine-1- carbonyl]phenyl]-1-[2,3-difluoro-4- (fluoromethoxy)phenyl]imidazole-4- carboxamide. 1:1 2,2,2-trifluoroacetic acid |
| 46 | 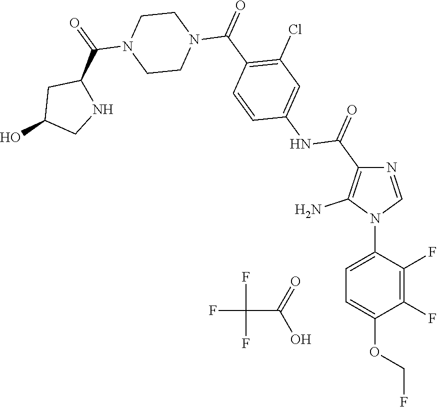 [SMILES: Nc1c(C(=O)Nc2ccc(C(=O)N3CCN(C(=O)[C@@H]4C[C@H](O)CN4)CC3)c(Cl)c2)ncn1-c1ccc(OCF)c(F)c1F ; O=C(O)C(F)(F)F] | 5-amino-N-[3-chloro-4-[4-[(2S,4S)-4- hydroxyprolyl]piperazine-1- carbonyl]phenyl]-1-[2,3-difluoro-4- (fluoromethoxy)phenyl]imidazole-4- carboxamide. 1:1 2,2,2-trifluoroacetic acid |
| 47 | 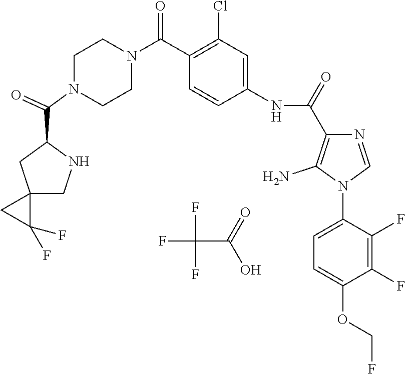 [SMILES: Nc1c(C(=O)Nc2ccc(C(=O)N3CCN(C(=O)[C@@H]4CC5(CN4)CC5(F)F)CC3)c(Cl)c2)ncn1-c1ccc(OCF)c(F)c1F ; O=C(O)C(F)(F)F] | 5-amino-N-[3-chloro-4-[4-[(6S)-2,2- difluoro-5-azaspiro[2.4]heptane-6- carbonyl]piperazine-1-carbonyl]phenyl]- 1-[2,3-difluoro-4- (fluoromethoxy)phenyl]imidazole-4- carboxamide. 1:1 2,2,2-trifluoroacetic acid |
| 48 | 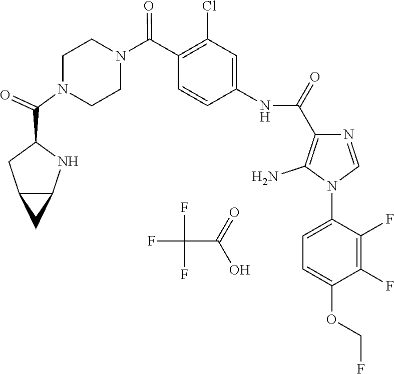 [SMILES: Nc1c(C(=O)Nc2ccc(C(=O)N3CCN(C(=O)[C@@H]4C[C@@H]5C[C@@H]5N4)CC3)c(Cl)c2)ncn1-c1ccc(OCF)c(F)c1F ; O=C(O)C(F)(F)F] | 5-amino-N-[4-[4-[(1S,3S,5S)-2- azabicyclo[3.1.0]hexane-3- carbonyl]piperazine-1-carbonyl]-3- chloro-phenyl]-1-[2,3-difluoro-4- (fluoromethoxy)phenyl]imidazole-4- carboxamide. 1:1 2,2,2-trifluoroacetic acid |
| 49 | 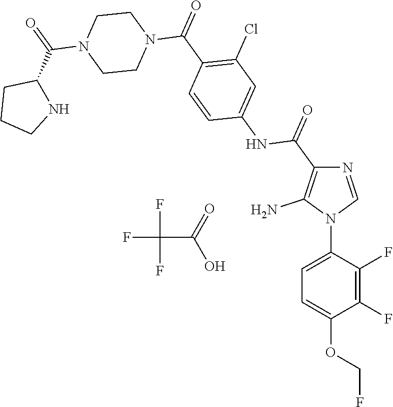 [SMILES: Nc1c(C(=O)Nc2ccc(C(=O)N3CCN(C(=O)[C@H]4CCCN4)CC3)c(Cl)c2)ncn1-c1ccc(OCF)c(F)c1F ; O=C(O)C(F)(F)F] | 5-amino-N-[3-chloro-4-[4-[(2R)- prolyl]piperazine-1-carbonyl]phenyl]-1- [2,3-difluoro-4- (fluoromethoxy)phenyl]imidazole-4- carboxamide. 1:1 2,2,2-trifluoroacetic acid |
| 50 | 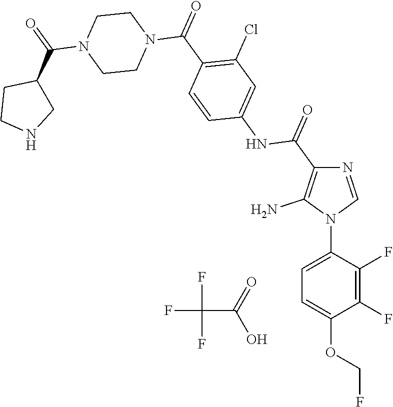 [SMILES: Nc1c(C(=O)Nc2ccc(C(=O)N3CCN(C(=O)[C@@H]4CCNC4)CC3)c(Cl)c2)ncn1-c1ccc(OCF)c(F)c1F ; O=C(O)C(F)(F)F] | 5-amino-N-[3-chloro-4-[4-[(3R)- pyrrolidine-3-carbonyl]piperazine-1- carbonyl]phenyl]-1-[2,3-difluoro-4- (fluoromethoxy)phenyl]imidazole-4- carboxamide. 1:1 2,2,2-trifluoroacetic acid |
| 51 | 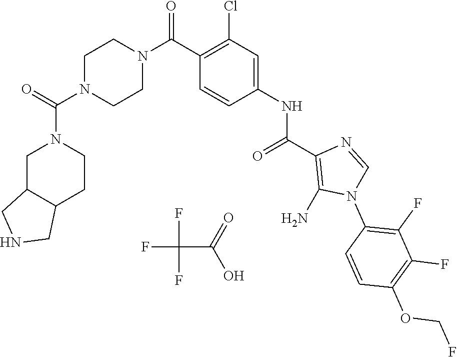 [SMILES: Nc1c(C(=O)Nc2ccc(C(=O)N3CCN(C(=O)N4CCC5CNCC5C4)CC3)c(Cl)c2)ncn1-c1ccc(OCF)c(F)c1F ; O=C(O)C(F)(F)F] | N-[4-[4-(1,2,3,3a,4,6,7,7a- octahydropyrrolo[3,4-c]pyridine-5- carbonyl)piperazine-1-carbonyl]-3- chloro-phenyl]-5-amino-1-[2,3-difluoro- 4-(fluoromethoxy)phenyl]imidazole-4- carboxamide. 1:1 2,2,2-trifluoroacetic acid |
| 52 | 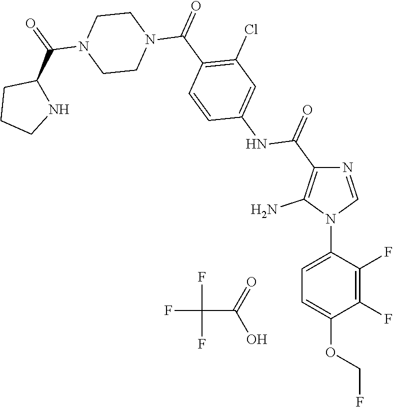 [SMILES: Nc1c(C(=O)Nc2ccc(C(=O)N3CCN(C(=O)[C@@H]4CCCN4)CC3)c(Cl)c2)ncn1-c1ccc(OCF)c(F)c1F ; O=C(O)C(F)(F)F] | 5-amino-N-[3-chloro-4-[4-[(2S)- prolyl]piperazine-1-carbonyl]phenyl]-1- [2,3-difluoro-4- (fluoromethoxy)phenyl]imidazole-4- carboxamide. 1:2 2,2,2-trifluoroacetic acid |
| 53 | 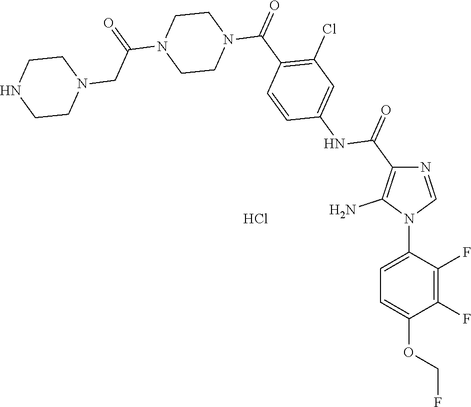 [SMILES: Nc1c(C(=O)Nc2ccc(C(=O)N3CCN(C(=O)CN4CCNCC4)CC3)c(Cl)c2)ncn1-c1ccc(OCF)c(F)c1F ; Cl] | 5-amino-N-[3-chloro-4-[4-(2- piperazinoacetyl)piperazine-1- carbonyl]phenyl]-1-[2,3-difluoro-4- (fluoromethoxy)phenyl]imidazole-4- carboxamide. 1:2 hydrogen chloride |
| 54 | 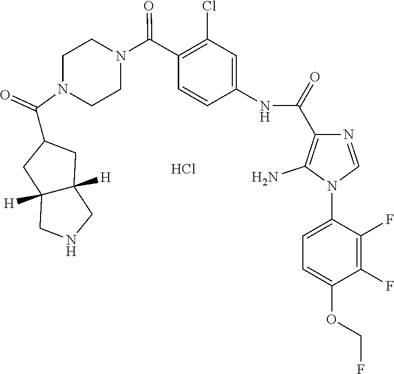 [SMILES: Nc1c(C(=O)Nc2ccc(C(=O)N3CCN(C(=O)C4C[C@H]5CNC[C@H]5C4)CC3)c(Cl)c2)ncn1-c1ccc(OCF)c(F)c1F ; Cl] | N-[4-[4-[(3aS,6aR)-1,2,3,3a,4,5,6,6a- octahydrocyclopenta[c]pyrrole-5- carbonyl]piperazine-1-carbonyl]-3- chloro-phenyl]-5-amino-1-[2,3-difluoro- 4-(fluoromethoxy)phenyl]imidazole-4- carboxamide. 1:2 hydrogen chloride |
| 55 | 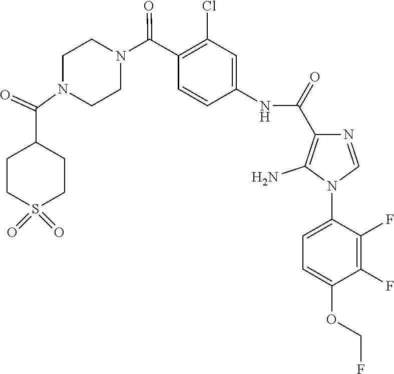 [SMILES: Nc1c(C(=O)Nc2ccc(C(=O)N3CCN(C(=O)C4CCS(=O)(=O)CC4)CC3)c(Cl)c2)ncn1-c1ccc(OCF)c(F)c1F] | 5-amino-N-[3-chloro-4-[4-(1,1- diketothiane-4-carbonyl)piperazine-1- carbonyl]phenyl]-1-[2,3-difluoro-4- (fluoromethoxy)phenyl]imidazole-4- carboxamide. 1:2 hydrogen chloride |
| 56 | 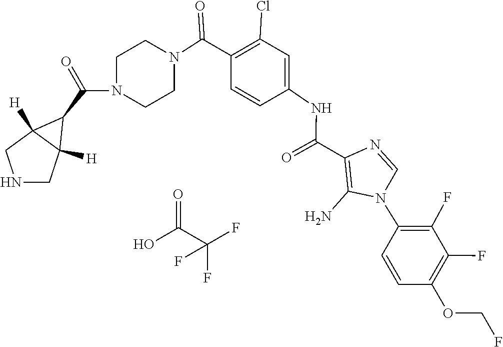 [SMILES: Nc1c(C(=O)Nc2ccc(C(=O)N3CCN(C(=O)[C@H]4[C@@H]5CNC[C@@H]54)CC3)c(Cl)c2)ncn1-c1ccc(OCF)c(F)c1F ; O=C(O)C(F)(F)F] | 5-amino-N-[4-[4-[(1R,5S,6r)-3- azabicyclo[3.1.0]hexane-6- carbonyl]piperazine-1-carbonyl]-3- chloro-phenyl]-1-[2,3-difluoro-4- (fluoromethoxy)phenyl]imidazole-4- carboxamide. .6:1 2,2,2-trifluoroacetic acid |
| 57 | 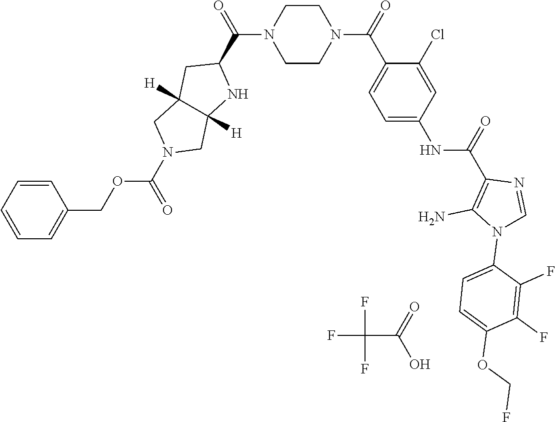 | (2S,3aS,6aS)-2-[4-[4-[5-amino-1-[2,3- difluoro-4- (fluoromethoxy)phenyl]imidazole-4- carbonyl]amino]-2-chloro- benzoyl]piperazine-1-carbonyl]- 2,3,3a,4,6,6a-hexahydro-1H-pyrrolo[2,3- c]pyrrole-5-carboxylic acid benzyl ester. 1:2 2,2,2-trifluoroacetic acid |
| 58 | 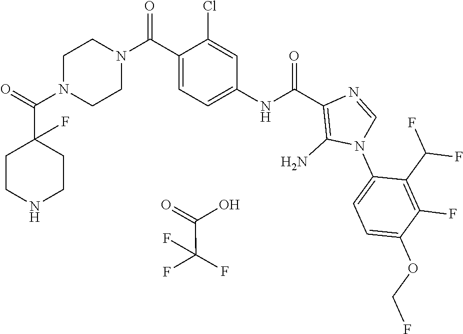 [SMILES: Nc1c(C(=O)Nc2ccc(C(=O)N3CCN(C(=O)C4(F)CCNCC4)CC3)c(Cl)c2)ncn1-c1ccc(OCF)c(F)c1C(F)F ; O=C(O)C(F)(F)F] | 5-amino-N-[3-chloro-4-[4-(4- fluoroisonipecotoyl)piperazine-1- carbonyl]phenyl]-1-[2-(difluoromethyl)-3- fluoro-4- (fluoromethoxy)phenyl]imidazole-4- carboxamide. 1:1 2,2,2-trifluoroacetic acid |
| 59 | 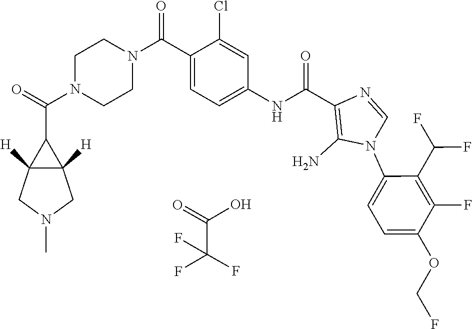 [SMILES: CN1C[C@@H]2C(C(=O)N3CCN(C(=O)c4ccc(NC(=O)c5ncn(-c6ccc(OCF)c(F)c6C(F)F)c5N)cc4Cl)CC3)[C@@H]2C1 ; O=C(O)C(F)(F)F] | 5-amino-N-[3-chloro-4-[4-[(1R,5S,6r)-3- methyl-3-azabicyclo[3.1.0]hexane-6- carbonyl]piperazine-1-carbonyl]phenyl]- 1-[2-(difluoromethyl)-3-fluoro-4- (fluoromethoxy)phenyl]imidazole-4- carboxamide. 1:1.38 2,2,2-trifluoroacetic acid |
| 60 | 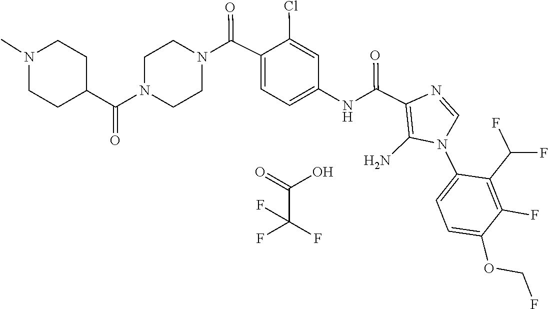 [SMILES: CN1CCC(C(=O)N2CCN(C(=O)c3ccc(NC(=O)c4ncn(-c5ccc(OCF)c(F)c5C(F)F)c4N)cc3Cl)CC2)CC1 ; O=C(O)C(F)(F)F] | 5-amino-N-[3-chloro-4-[4-(1- methylisonipecotoyl)piperazine-1- carbonyl]phenyl]-1-[2-(difluoromethyl)-3- fluoro-4- (fluoromethoxy)phenyl]imidazole-4- carboxamide. 1:1.34 2,2,2-trifluoroacetic acid |
| 61 | 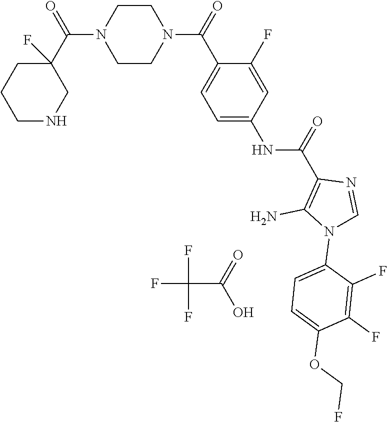 [SMILES: Nc1c(C(=O)Nc2ccc(C(=O)N3CCN(C(=O)C4(F)CCCNC4)CC3)c(F)c2)ncn1-c1ccc(OCF)c(F)c1F ; O=C(O)C(F)(F)F] | 5-amino-1-[2,3-difluoro-4- (fluoromethoxy)phenyl]-N-[3-fluoro-4-[4- (3-fluoronipecotoyl)piperazine-1- carbonyl]phenyl]imidazole-4- carboxamide. 1:1 2,2,2-trifluoroacetic acid |
| 62 | 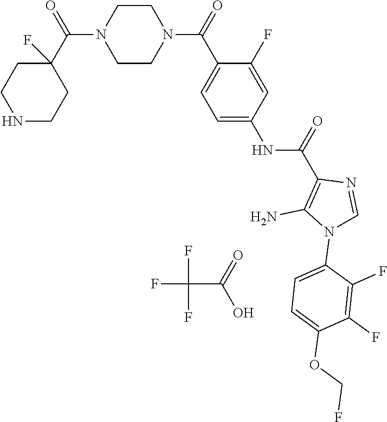 [SMILES: Nc1c(C(=O)Nc2ccc(C(=O)N3CCN(C(=O)C4(F)CCNCC4)CC3)c(F)c2)ncn1-c1ccc(OCF)c(F)c1F ; O=C(O)C(F)(F)F] | 5-amino-1-[2,3-difluoro-4- (fluoromethoxy)phenyl]-N-[3-fluoro-4-[4- (4-fluoroisonipecotoyl)piperazine-1- carbonyl]phenyl]imidazole-4- carboxamide. 1:2 2,2,2-trifluoroacetic acid |
| 63 | 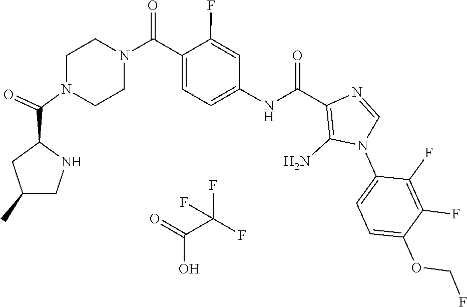 [SMILES: C[C@@H]1CN[C@H](C(=O)N2CCN(C(=O)c3ccc(NC(=O)c4ncn(-c5ccc(OCF)c(F)c5F)c4N)cc3F)CC2)C1 ; O=C(O)C(F)(F)F] | 5-amino-1-[2,3-difluoro-4- (fluoromethoxy)phenyl]-N-[3-fluoro-4-[4- [(2S,4S)-4-methylprolyl]piperazine-1- carbonyl]phenyl]imidazole-4- carboxamide. 1:1.3 2,2,2-trifluoroacetic acid |
| 64 | 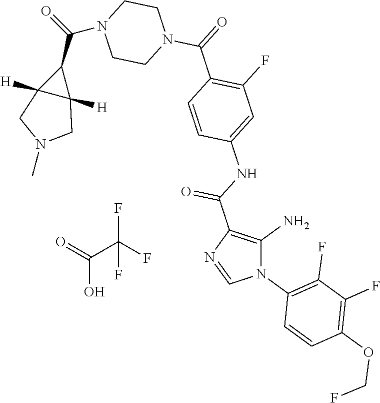 [SMILES: CN1C[C@@H]2[C@H](C1)[C@H]2C(=O)N1CCN(C(=O)c2ccc(NC(=O)c3ncn(-c4ccc(OCF)c(F)c4F)c3N)cc2F)CC1 ; O=C(O)C(F)(F)F] | 5-amino-1-[2,3-difluoro-4- (fluoromethoxy)phenyl]-N-[3-fluoro-4-[4- [(1R,5S,6r)-3-methyl-3- azabicyclo[3.1.0]hexane-6- carbonyl]piperazine-1- carbonyl]phenyl]imidazole-4- carboxamide. 1:1.37 2,2,2-trifluoroacetic acid |
| 65 | 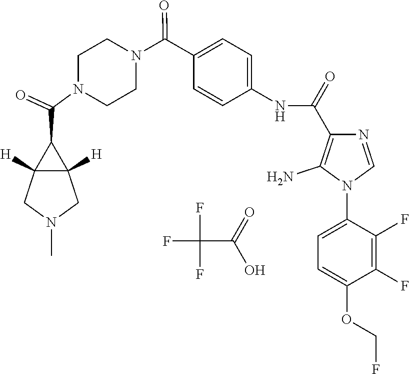 [SMILES: CN1C[C@@H]2[C@H](C1)[C@H]2C(=O)N1CCN(C(=O)c2ccc(NC(=O)c3ncn(-c4ccc(OCF)c(F)c4F)c3N)cc2)CC1 ; O=C(O)C(F)(F)F] | 5-amino-1-[2,3-difluoro-4- (fluoromethoxy)phenyl]-N-[4-[4- [(1R,5S,6r)-3-methyl-3- azabicyclo[3.1.0]hexane-6- carbonyl]piperazine-1- carbonyl]phenyl]imidazole-4- carboxamide. 1:1 2,2,2-trifluoroacetic acid |
| 66 | 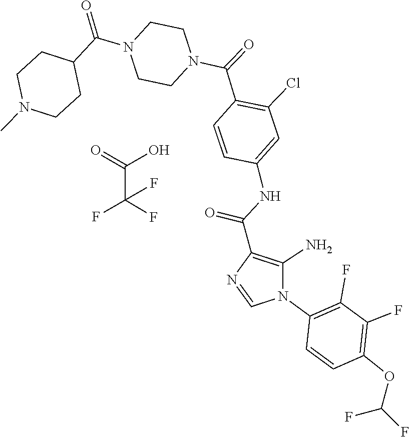 [SMILES: CN1CCC(C(=O)N2CCN(C(=O)c3ccc(NC(=O)c4ncn(-c5ccc(OC(F)F)c(F)c5F)c4N)cc3Cl)CC2)CC1 ; O=C(O)C(F)(F)F] | 5-amino-N-[3-chloro-4-[4-(1- methylisonipecotoyl)piperazine-1- carbonyl]phenyl]-1-[4-(difluoromethoxy)- 2,3-difluoro-phenyl]imidazole-4- carboxamide. 1:1.6 2,2,2-trifluoroacetic acid |
| 67 | 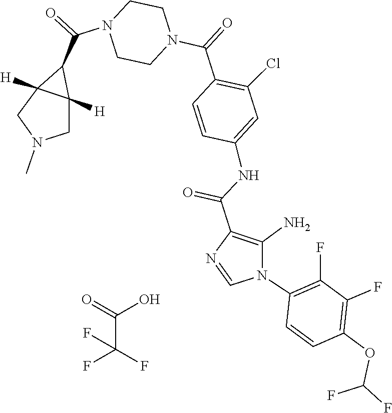 [SMILES: CN1C[C@@H]2[C@H](C1)[C@H]2C(=O)N1CCN(C(=O)c2ccc(NC(=O)c3ncn(-c4ccc(OC(F)F)c(F)c4F)c3N)cc2Cl)CC1 ; O=C(O)C(F)(F)F] | 5-amino-N-[3-chloro-4-[4-[(1R,5S,6r)-3- methyl-3-azabicyclo[3.1.0]hexane-6- carbonyl]piperazine-1-carbonyl]phenyl]- 1-[4-(difluoromethoxy)-2,3-difluoro- phenyl]imidazole-4-carboxamide. 1:1.2 2,2,2-trifluoroacetic acid |
| 68 | 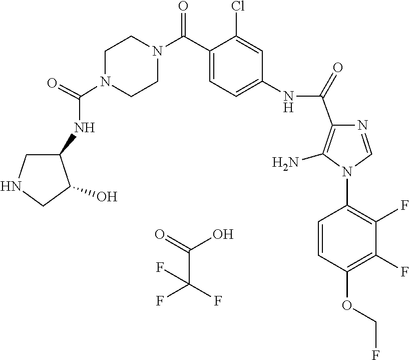 [SMILES: Nc1c(C(=O)Nc2ccc(C(=O)N3CCN(C(=O)N[C@@H]4CNC[C@H]4O)CC3)c(Cl)c2)ncn1-c1ccc(OCF)c(F)c1F ; O=C(O)C(F)(F)F] | Rac-4-[4-[[5-amino-1-[2,3-difluoro-4- (fluoromethoxy)phenyl]imidazole-4- carbonyl]amino]-2-chloro-benzoyl]-N- [(3R,4R)-4-hydroxypyrrolidin-3- yl]piperazine-1-carboxamide. 1:1.4 2,2,2-trifluoroacetic acid |
| 69 | 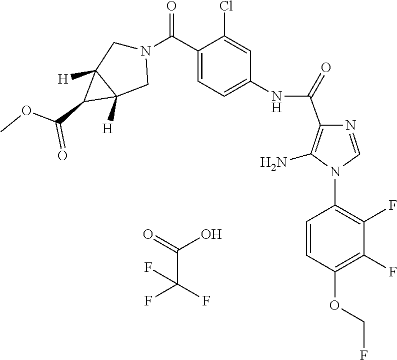 [SMILES: COC(=O)[C@H]1[C@@H]2CN(C(=O)c3ccc(NC(=O)c4ncn(-c5ccc(OCF)c(F)c5F)c4N)cc3Cl)C[C@@H]21 ; O=C(O)C(F)(F)F] | (1R,5S,6r)-3-[4-[[5-amino-1-[2,3- difluoro-4- (fluoromethoxy)phenyl]imidazole-4- carbonyl]amino]-2-chloro-benzoyl]-3- azabicyclo[3.1.0]hexane-6-carboxylic acid methyl ester. 1:.6 2,2,2- trifluoroacetic acid |
| 70 | 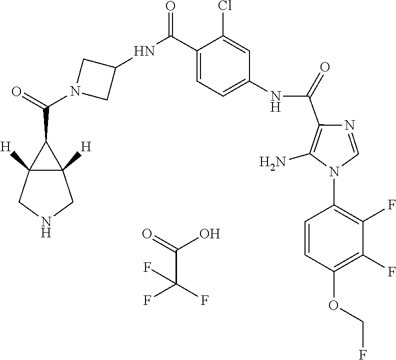 [SMILES: Nc1c(C(=O)Nc2ccc(C(=O)NC3CN(C(=O)[C@H]4[C@@H]5CNC[C@@H]54)C3)c(Cl)c2)ncn1-c1ccc(OCF)c(F)c1F ; O=C(O)C(F)(F)F] | 5-amino-N-[4-[1-[(1R,5S,6r)-3- azabicyclo[3.1.0]hexane-6- carbonyl]azetidin-3-yl]carbamoyl]-3- chloro-phenyl]-1-[2,3-difluoro-4- (fluoromethoxy)phenyl]imidazole-4- carboxamide. 1:1.3 2,2,2-trifluoroacetic acid |
| 71 | 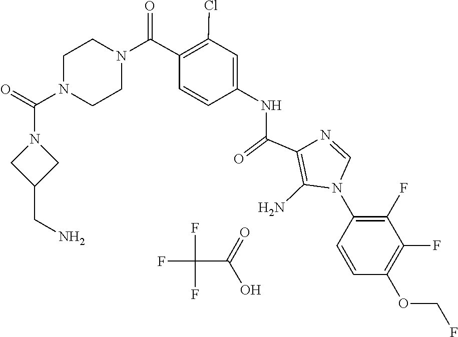 | 5-amino-N-[4-[4-[3- (aminomethyl)azetidine-1- carbonyl]piperazine-1-carbonyl]-3- chloro-phenyl]-1-[2,3-difluoro-4- (fluoromethoxy)phenyl]imidazole-4- carboxamide. 1:1.2 2,2,2-trifluoroacetic acid |
| 72 | 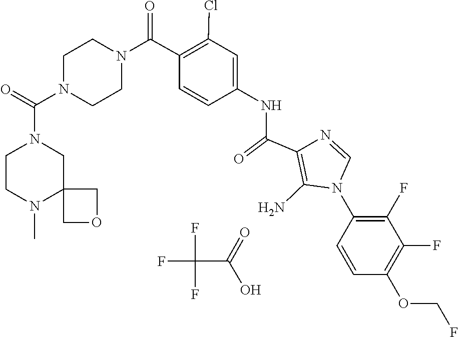 | 5-amino-N-[3-chloro-4-[4-(5-methyl-2- oxa-5,8-diazaspiro[3.5]nonane-8- carbonyl)piperazine-1-carbonyl]phenyl]- 1-[2,3-difluoro-4- (fluoromethoxy)phenyl]imidazole-4- carboxamide. 1:1.3 2,2,2-trifluoroacetic acid |
| 73 | 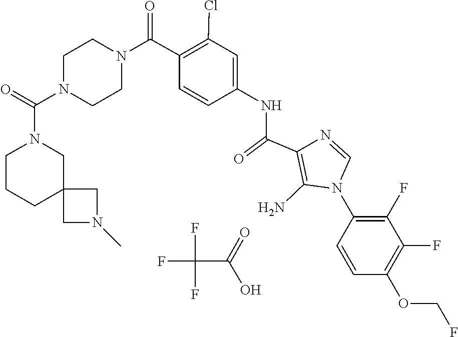 | 5-amino-N-[3-chloro-4-[4-(2-methyl-2,6- diazaspiro[3.5]nonane-6- carbonyl)piperazine-1-carbonyl]phenyl]- 1-[2,3-difluoro-4- (fluoromethoxy)phenyl]imidazole-4- carboxamide. 1:1.3 2,2,2-trifluoroacetic acid |
| 74 | 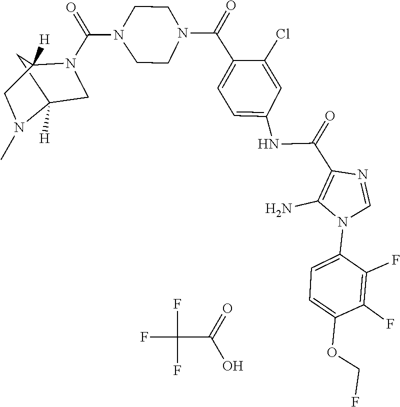 [SMILES: CN1C[C@@H]2C[C@H]1CN2C(=O)N1CCN(C(=O)c2ccc(NC(=O)c3ncn(-c4ccc(OCF)c(F)c4F)c3N)cc2Cl)CC1 ; O=C(O)C(F)(F)F] | 5-amino-N-[3-chloro-4-[4-[(1S,4S)-5- methyl-2,5-diazabicyclo[2.2.1]heptane- 2-carbonyl]piperazine-1- carbonyl]phenyl]-1-[2,3-difluoro-4- (fluoromethoxy)phenyl]imidazole-4- carboxamide. 1:2 2,2,2-trifluoroacetic acid |
| 75 | 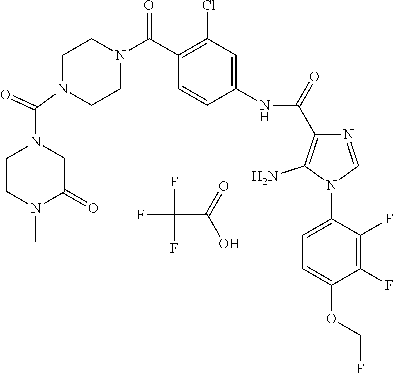 [SMILES: CN1CCN(C(=O)N2CCN(C(=O)c3ccc(NC(=O)c4ncn(-c5ccc(OCF)c(F)c5F)c4N)cc3Cl)CC2)CC1=O ; O=C(O)C(F)(F)F] | 5-amino-N-[3-chloro-4-[4-(3-keto-4- methyl-piperazine-1- carbonyl)piperazine-1-carbonyl]phenyl]- 1-[2,3-difluoro-4- (fluoromethoxy)phenyl]imidazole-4- carboxamide. 1:1 2,2,2-trifluoroacetic acid |
| 76 | 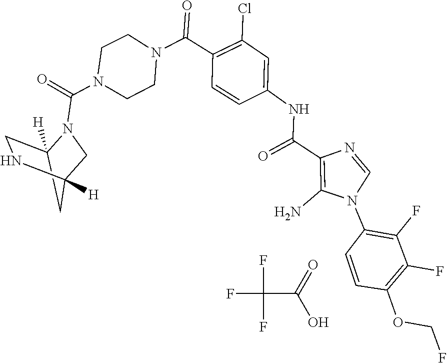 | 5-amino-N-[3-chloro-4-[4-[(1S,4S)-2,5- diazabicyclo[2.2.1]heptane-2- carbonyl]piperazine-1-carbonyl]phenyl]- 1-[2,3-difluoro-4- (fluoromethoxy)phenyl]imidazole-4- carboxamide. 1:2 2,2,2-trifluoroacetic acid |
| 77 | 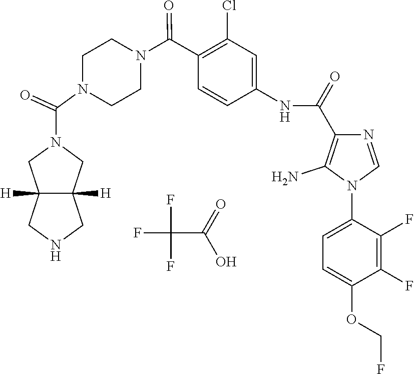 [SMILES: Nc1c(C(=O)Nc2ccc(C(=O)N3CCN(C(=O)N4C[C@H]5CNC[C@H]5C4)CC3)c(Cl)c2)ncn1-c1ccc(OCF)c(F)c1F ; O=C(O)C(F)(F)F] | N-[4-[4-[(3aS,6aR)-2,3,3a,4,6,6a- hexahydro-1H-pyrrolo[3,4-c]pyrrole-5- carbonyl]piperazine-1-carbonyl]-3- chloro-phenyl]-5-amino-1-[2,3-difluoro- 4-(fluoromethoxy)phenyl]imidazole-4- carboxamide. 1:1 2,2,2-trifluoroacetic acid |
| 78 | 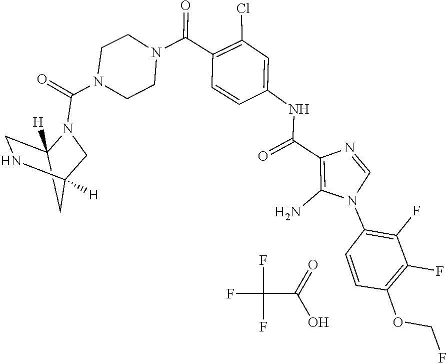 | 5-amino-N-[3-chloro-4-[4-[(1R,4R)-2,5- diazabicyclo[2.2.1]heptane-2- carbonyl]piperazine-1-carbonyl]phenyl]- 1-[2,3-difluoro-4- (fluoromethoxy)phenyl]imidazole-4- carboxamide. 1:1 2,2,2-trifluoroacetic acid |
| 79 | 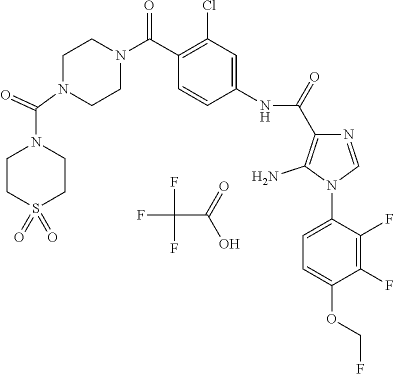 [SMILES: Nc1c(C(=O)Nc2ccc(C(=O)N3CCN(C(=O)N4CCS(=O)(=O)CC4)CC3)c(Cl)c2)ncn1-c1ccc(OCF)c(F)c1F ; O=C(O)C(F)(F)F] | 5-amino-N-[3-chloro-4-[4-(1,1-diketo- 1,4-thiazinane-4-carbonyl)piperazine-1- carbonyl]phenyl]-1-[2,3-difluoro-4- (fluoromethoxy)phenyl]imidazole-4- carboxamide. 1:1 2,2,2-trifluoroacetic acid |
| 80 | 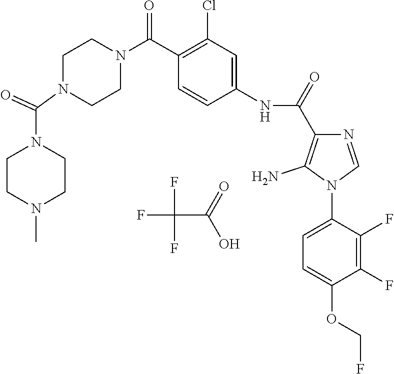 [SMILES: CN1CCN(C(=O)N2CCN(C(=O)c3ccc(NC(=O)c4ncn(-c5ccc(OCF)c(F)c5F)c4N)cc3Cl)CC2)CC1 ; O=C(O)C(F)(F)F] | 5-amino-N-[3-chloro-4-[4-(4- methylpiperazine-1-carbonyl)piperazine- 1-carbonyl]phenyl]-1-[2,3-difluoro-4- (fluoromethoxy)phenyl]imidazole-4- carboxamide. 1:1 2,2,2-trifluoroacetic acid |
| 81 | 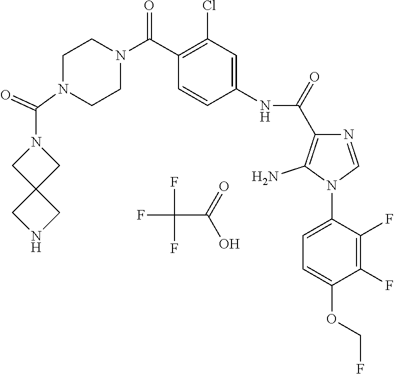 [SMILES: Nc1c(C(=O)Nc2ccc(C(=O)N3CCN(C(=O)N4CC5(CNC5)C4)CC3)c(Cl)c2)ncn1-c1ccc(OCF)c(F)c1F ; O=C(O)C(F)(F)F] | 5-amino-N-[3-chloro-4-[4-(2,6- diazaspiro[3.3]heptane-2- carbonyl)piperazine-1-carbonyl]phenyl]- 1-[2,3-difluoro-4- (fluoromethoxy)phenyl]imidazole-4- carboxamide. 1:1 2,2,2-trifluoroacetic acid |
| 82 | 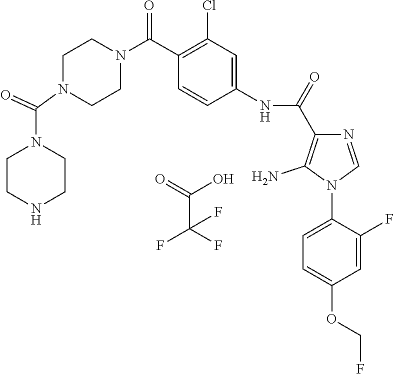 [SMILES: Nc1c(C(=O)Nc2ccc(C(=O)N3CCN(C(=O)N4CCNCC4)CC3)c(Cl)c2)ncn1-c1ccc(OCF)cc1F ; O=C(O)C(F)(F)F] | 5-amino-N-[3-chloro-4-[4-(piperazine-1- carbonyl)piperazine-1-carbonyl]phenyl]- 1-[2-fluoro-4- (fluoromethoxy)phenyl]imidazole-4- carboxamide. 1:1.5 2,2,2-trifluoroacetic acid |
| 83 | 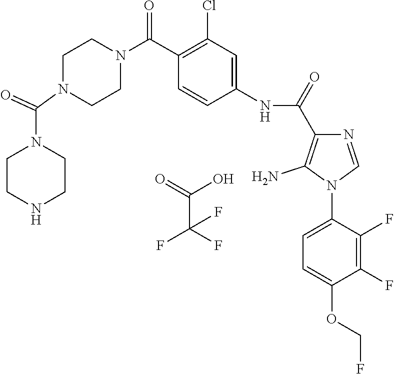 [SMILES: Nc1c(C(=O)Nc2ccc(C(=O)N3CCN(C(=O)N4CCNCC4)CC3)c(Cl)c2)ncn1-c1ccc(OCF)c(F)c1F ; O=C(O)C(F)(F)F] | 5-amino-N-[3-chloro-4-[4-(piperazine-1- carbonyl)piperazine-1-carbonyl]phenyl]- 1-[2,3-difluoro-4- (fluoromethoxy)phenyl]imidazole-4- carboxamide. 1:1.2 2,2,2-trifluoroacetic acid |
| 84 | 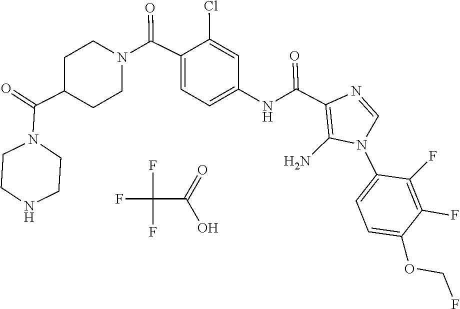 | 5-amino-N-[3-chloro-4-[4-(piperazine-1- carbonyl)piperidine-1-carbonyl]phenyl]- 1-[2,3-difluoro-4- (fluoromethoxy)phenyl]imidazole-4- carboxamide. 1:2 2,2,2-trifluoroacetic acid |
| 85 | 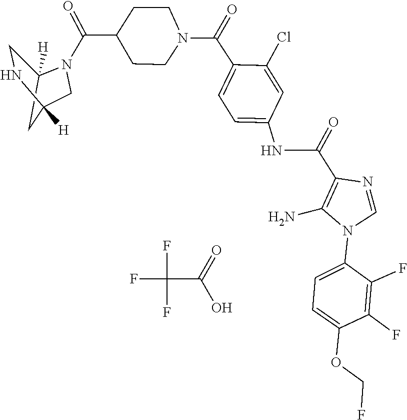 [SMILES: Nc1c(C(=O)Nc2ccc(C(=O)N3CCC(C(=O)N4C[C@@H]5C[C@H]4CN5)CC3)c(Cl)c2)ncn1-c1ccc(OCF)c(F)c1F ; O=C(O)C(F)(F)F] | 5-amino-N-[3-chloro-4-[4-[(1S,4S)-2,5- diazabicyclo[2.2.1]heptane-2- carbonyl]piperidine-1-carbonyl]phenyl]- 1-[2,3-difluoro-4- (fluoromethoxy)phenyl]imidazole-4- carboxamide. 1:2 2,2,2-trifluoroacetic acid |
| 86 | 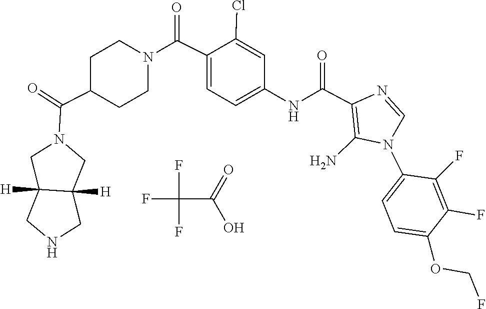 | N-[4-[4-[(3aS,6aR)-2,3,3a,4,6,6a- hexahydro-1H-pyrrolo[3,4-c]pyrrole-5- carbonyl]piperidine-1-carbonyl]-3- chloro-phenyl]-5-amino-1-[2,3-difluoro- 4-(fluoromethoxy)phenyl]imidazole-4- carboxamide. 1:2 2,2,2-trifluoroacetic acid |
| 87 | 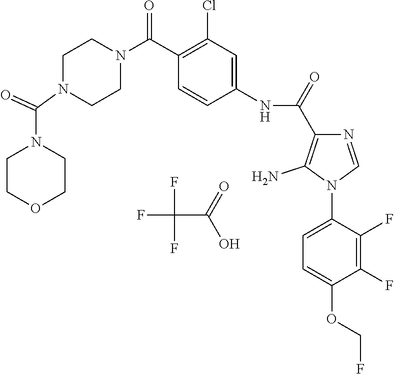 [SMILES: Nc1c(C(=O)Nc2ccc(C(=O)N3CCN(C(=O)N4CCOCC4)CC3)c(Cl)c2)ncn1-c1ccc(OCF)c(F)c1F ; O=C(O)C(F)(F)F] | 5-amino-N-[3-chloro-4-[4-(morpholine-4- carbonyl)piperazine-1-carbonyl]phenyl]- 1-[2,3-difluoro-4- (fluoromethoxy)phenyl]imidazole-4- carboxamide. 1:1 2,2,2-trifluoroacetic acid |
| 88 | 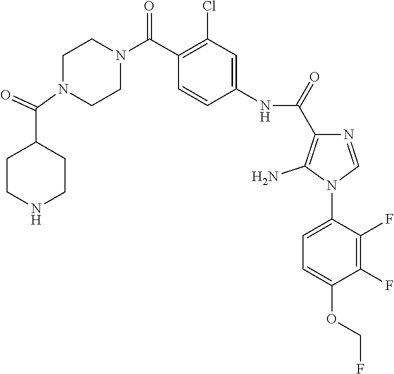 [SMILES: Nc1c(C(=O)Nc2ccc(C(=O)N3CCN(C(=O)C4CCNCC4)CC3)c(Cl)c2)ncn1-c1ccc(OCF)c(F)c1F] | 5-amino-N-[3-chloro-4-(4- isonipecotoylpiperazine-1- carbonyl)phenyl]-1-[2,3-difluoro-4- (fluoromethoxy)phenyl]imidazole-4- carboxamide |
| 89 | 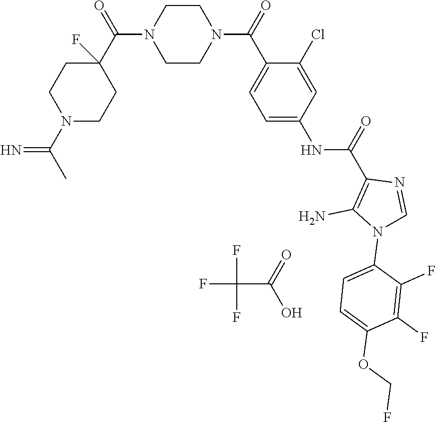 [SMILES: CC(=N)N1CCC(F)(C(=O)N2CCN(C(=O)c3ccc(NC(=O)c4ncn(-c5ccc(OCF)c(F)c5F)c4N)cc3Cl)CC2)CC1 ; O=C(O)C(F)(F)F] | N-[4-[4-(1-acetimidoyl-4-fluoro- isonipecotoyl)piperazine-1-carbonyl]-3- chloro-phenyl]-5-amino-1-[2,3-difluoro- 4-(fluoromethoxy)phenyl]imidazole-4- carboxamide. 1:2 2,2,2-trifluoroacetic acid |
| 90 | 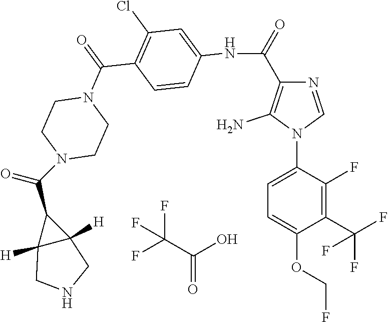 | 5-amino-N-[4-[4-[(1R,5S,6r)-3- azabicyclo[3.1.0]hexane-6- carbonyl]piperazine-1-carbonyl]-3- chloro-phenyl]-1-[2-fluoro-4- (fluoromethoxy)-3- (trifluoromethyl)phenyl]imidazole-4- carboxamide. 1:1.3 2,2,2-trifluoroacetic acid |
| 91 | 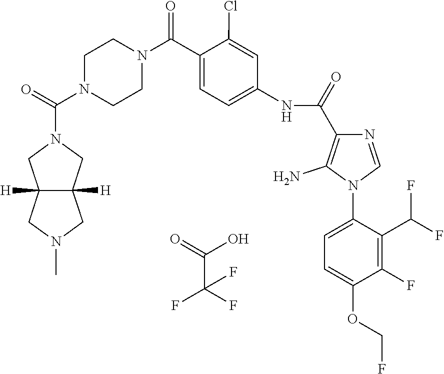 [SMILES: CN1C[C@@H]2CN(C(=O)N3CCN(C(=O)c4ccc(NC(=O)c5ncn(-c6ccc(OCF)c(F)c6C(F)F)c5N)cc4Cl)CC3)C[C@@H]2C1 ; O=C(O)C(F)(F)F] | N-[4-[4-[(3aS,6aR)-2-methyl- 1,3,3a,4,6,6a-hexahydropyrrolo[3,4- c]pyrrole-5-carbonyl]piperazine-1- carbonyl]-3-chloro-phenyl]-5-amino-1- [2-(difluoromethyl)-3-fluoro-4- (fluoromethoxy)phenyl]imidazole-4- carboxamide. 1:1 2,2,2-trifluoroacetic acid |
| 92 | 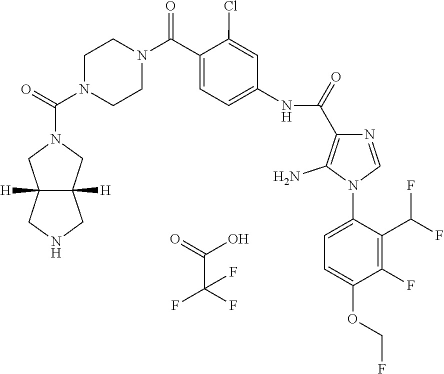 [SMILES: Nc1c(C(=O)Nc2ccc(C(=O)N3CCN(C(=O)N4C[C@H]5CNC[C@H]5C4)CC3)c(Cl)c2)ncn1-c1ccc(OCF)c(F)c1C(F)F ; O=C(O)C(F)(F)F] | N-[4-[4-[(3aS,6aR)-2,3,3a,4,6,6a- hexahydro-1H-pyrrolo[3,4-c]pyrrole-5- carbonyl]piperazine-1-carbonyl]-3- chloro-phenyl]-5-amino-1-[2- (difluoromethyl)-3-fluoro-4- (fluoromethoxy)phenyl]imidazole-4- carboxamide. 1:1.3 2,2,2-trifluoroacetic acid |
| 93 | 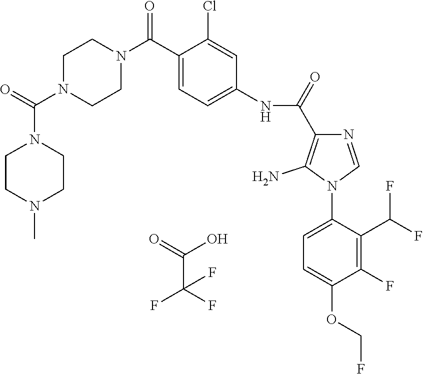 [SMILES: CN1CCN(C(=O)N2CCN(C(=O)c3ccc(NC(=O)c4ncn(-c5ccc(OCF)c(F)c5C(F)F)c4N)cc3Cl)CC2)CC1 ; O=C(O)C(F)(F)F] | 5-amino-N-[3-chloro-4-[4-(4- methylpiperazine-1-carbonyl)piperazine- 1-carbonyl]phenyl]-1-[2-(difluoromethyl)- 3-fluoro-4- (fluoromethoxy)phenyl]imidazole-4- carboxamide. 1:1.61 2,2,2-trifluoroacetic acid |
| 94 | 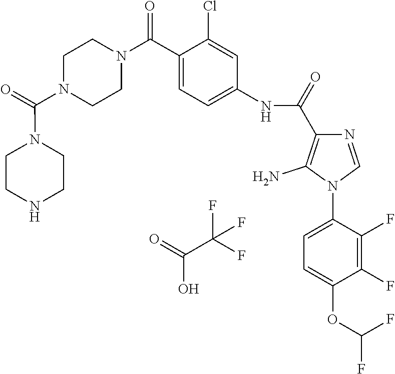 [SMILES: Nc1c(C(=O)Nc2ccc(C(=O)N3CCN(C(=O)N4CCNCC4)CC3)c(Cl)c2)ncn1-c1ccc(OC(F)F)c(F)c1F ; O=C(O)C(F)(F)F] | 5-amino-N-[3-chloro-4-[4-(piperazine-1- carbonyl)piperazine-1-carbonyl]phenyl]- 1-[4-(difluoromethoxy)-2,3-difluoro- phenyl]imidazole-4-carboxamide. 1:1.3 2,2,2-trifluoroacetic acid |
| 95 | 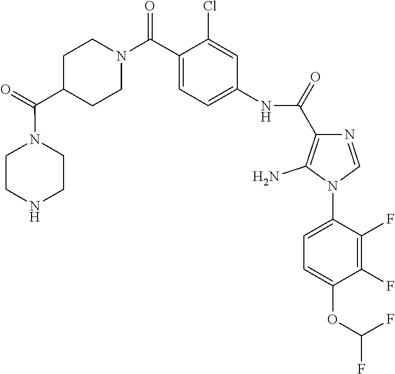 [SMILES: Nc1c(C(=O)Nc2ccc(C(=O)N3CCC(C(=O)N4CCNCC4)CC3)c(Cl)c2)ncn1-c1ccc(OC(F)F)c(F)c1F] | 5-amino-N-[3-chloro-4-[4-(piperazine-1- carbonyl)piperidine-1-carbonyl]phenyl]- 1-[4-(difluoromethoxy)-2,3-difluoro- phenyl]imidazole-4-carboxamide |
| 96 | 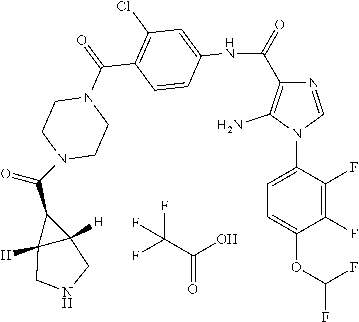 | 5-amino-N-[4-[4-[(1R,5S,6r)-3- azabicyclo[3.1.0]hexane-6- carbonyl]piperazine-1-carbonyl]-3- chloro-phenyl]-1-[4-(difluoromethoxy)- 2,3-difluoro-phenyl]imidazole-4- carboxamide. 1:1 2,2,2-trifluoroacetic acid |
| 97 | 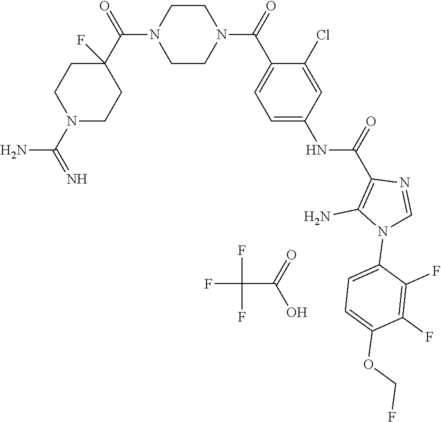 [SMILES: N=C(N)N1CCC(F)(C(=O)N2CCN(C(=O)c3ccc(NC(=O)c4ncn(-c5ccc(OCF)c(F)c5F)c4N)cc3Cl)CC2)CC1 ; O=C(O)C(F)(F)F] | N-[4-[4-(1-amidino-4-fluoro- isonipecotoyl)piperazine-1-carbonyl]-3- chloro-phenyl]-5-amino-1-[2,3-difluoro- 4-(fluoromethoxy)phenyl]imidazole-4- carboxamide. 1:2 2,2,2-trifluoroacetic acid |
| 98 | 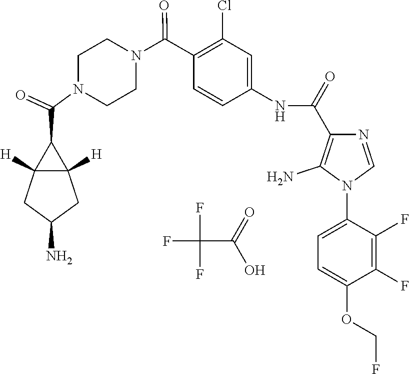 [SMILES: Nc1c(C(=O)Nc2ccc(C(=O)N3CCN(C(=O)[C@H]4[C@@H]5C[C@@H](N)C[C@@H]54)CC3)c(Cl)c2)ncn1-c1ccc(OCF)c(F)c1F ; O=C(O)C(F)(F)F] | 5-amino-N-[4-[4-[(1R,3s,5S,6r)-3- aminobicyclo[3.1.0]hexane-6- carbonyl]piperazine-1-carbonyl]-3- chloro-phenyl]-1-[2,3-difluoro-4- (fluoromethoxy)phenyl]imidazole-4- carboxamide. 1:1 2,2,2-trifluoroacetic acid |
| 99 | 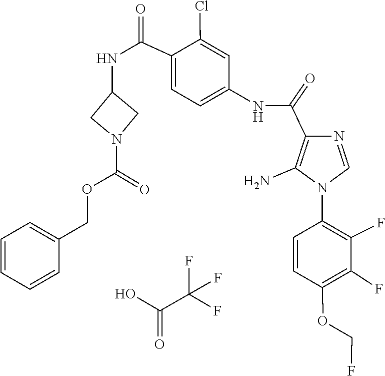 [SMILES: Nc1c(C(=O)Nc2ccc(C(=O)NC3CN(C(=O)OCc4ccccc4)C3)c(Cl)c2)ncn1-c1ccc(OCF)c(F)c1F ; O=C(O)C(F)(F)F] | 3-[[4-[[5-amino-1-[2,3-difluoro-4- (fluoromethoxy)phenyl]imidazole-4- carbonyl]amino]-2-chloro- benzoyl]amino]azetidine-1-carboxylic acid benzyl ester. 1:.5 2,2,2- trifluoroacetic acid |
| 100 | 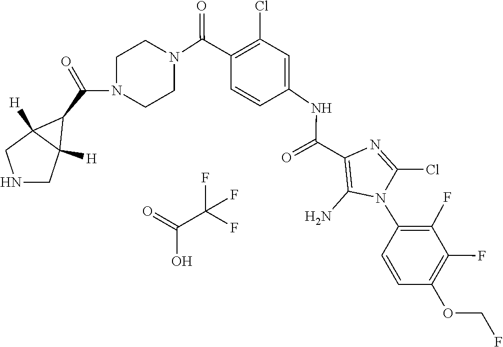 [SMILES: Nc1c(C(=O)Nc2ccc(C(=O)N3CCN(C(=O)[C@H]4[C@@H]5CNC[C@@H]54)CC3)c(Cl)c2)nc(Cl)n1-c1ccc(OCF)c(F)c1F ; O=C(O)C(F)(F)F] | 5-amino-N-[4-[4-[(1R,5S,6r)-3- azabicyclo[3.1.0]hexane-6- carbonyl]piperazine-1-carbonyl]-3- chloro-phenyl]-2-chloro-1-[2,3-difluoro- 4-(fluoromethoxy)phenyl]imidazole-4- carboxamide. 1:1 2,2,2-trifluoroacetic acid |
| 101 | 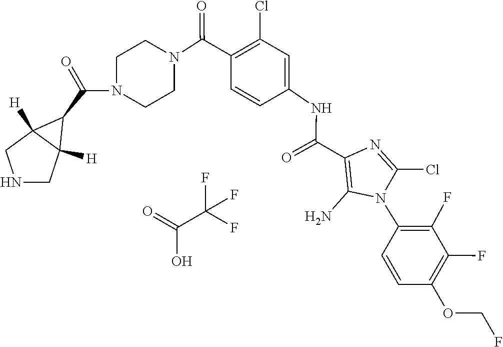 [SMILES: Nc1c(C(=O)Nc2ccc(C(=O)N3CCN(C(=O)[C@H]4[C@@H]5CNC[C@@H]54)CC3)c(Cl)c2)nc(Cl)n1-c1ccc(OCF)c(F)c1F ; O=C(O)C(F)(F)F] | 5-amino-N-[4-[4-[(1R,5S,6r)-3- azabicyclo[3.1.0]hexane-6- carbonyl]piperazine-1-carbonyl]-3- chloro-phenyl]-2-chloro-1-[2-fluoro-4- (fluoromethoxy)phenyl]imidazole-4- carboxamide. 1:1 2,2,2-trifluoroacetic acid |
| 102 | 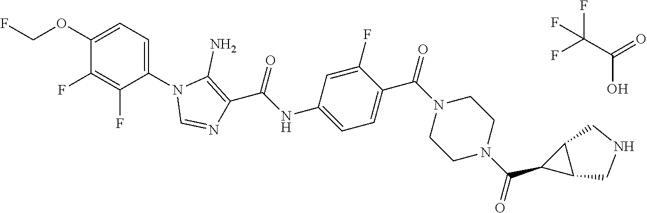 | 5-Amino-N-[4-[4-[(1R,5S,6r)-3- azabicyclo[3.1.0]hexane-6- carbonyl]piperazine-1-carbonyl]-3- fluoro-phenyl]-1-[2,3-difluoro-4- (fluoromethoxy)phenyl]imidazole-4- carboxamide; 1:1 2,2,2-trifluoroacetic acid |
| 103 | 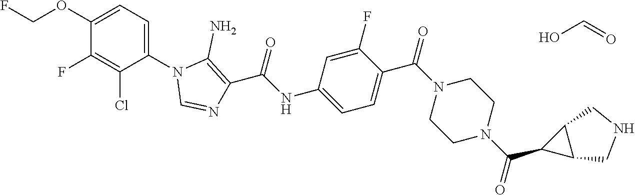 [SMILES: Nc1c(C(=O)Nc2ccc(C(=O)N3CCN(C(=O)[C@H]4[C@@H]5CNC[C@@H]54)CC3)c(F)c2)ncn1-c1ccc(OCF)c(F)c1Cl ; O=CO] | 5-Amino-N-[4-[4-[(1R,5S,6r)-3- azabicyclo [3.1.0] hexane-6-carbonyl] piperazine-1-carbonyl]-3-fluoro-phenyl]- 1-[2-chloro-3-fluoro-4-(fluoromethoxy) phenyl] imidazole-4-carboxamide; 1:1 formic acid |
| 104 | 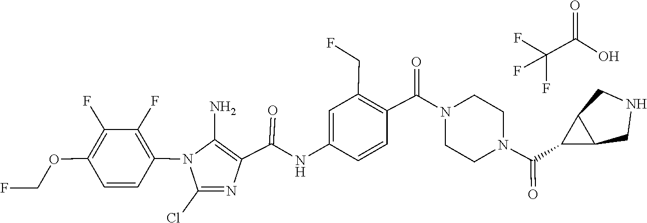 | 5-amino-N-[4-[4-[(1S,5R,6r)-3- azabicyclo[3.1.0]hexane-6- carbonyl]piperazine-1-carbonyl]-3- (fluoromethyl)phenyl]-2-chloro-1-[2,3- difluoro-4- (fluoromethoxy)phenyl]imidazole-4- carboxamide; 1:1 2,2,2-trifluoroacetic acid |
| 105 | 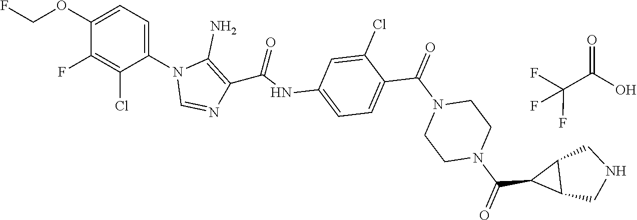 | 5-amino-N-[4-[4-[(1R,5S,6r)-3- azabicyclo[3.1.0]hexane-6- carbonyl]piperazine-1-carbonyl]-3- chloro-phenyl]-1-[2-chloro-3-fluoro-4- (fluoromethoxy)phenyl]imidazole-4- carboxamide; 1:1 2,2,2-trifluoroacetic acid |
| 106 | 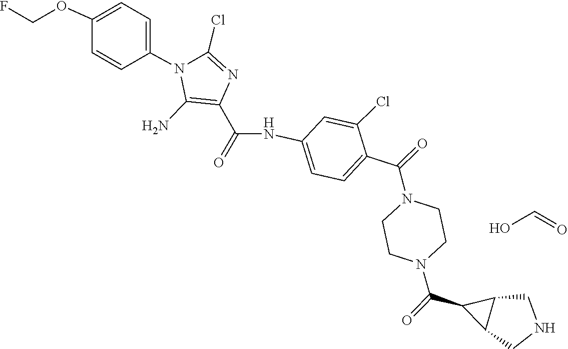 [SMILES: Nc1c(C(=O)Nc2ccc(C(=O)N3CCN(C(=O)[C@H]4[C@@H]5CNC[C@@H]54)CC3)c(Cl)c2)nc(Cl)n1-c1ccc(OCF)cc1 ; O=CO] | 5-Amino-N-[4-[4-[(1R,5S,6r)-3- azabicyclo[3.1.0]hexane-6- carbonyl]piperazine-1-carbonyl]-3- chloro-phenyl]-2-chloro-1-[4- (fluoromethoxy)phenyl]imidazole-4- carboxamide; 1:1 formic acid |
| 107 | 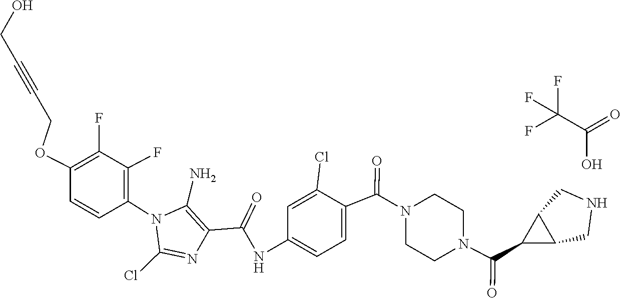 | 5-amino-2-chloro-N-[3-chloro-4-[4- [(1R,5S,6r)-3-azabicyclo[3.1.0]hexane-6- carbonyl]piperazine-1-carbonyl]phenyl]- 1-[2,3-difluoro-4-(4-hydroxybut-2- ynoxy)phenyl]imidazole-4-carboxamide; 1:1 2,2,2-trifluoroacetic acid |
| 108 | 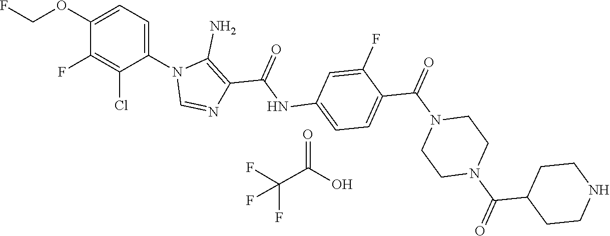 | 5-amino-1-[2-chloro-3-fluoro-4- (fluoromethoxy) phenyl]-N-[3-fluoro-4- [4-(piperidine-4-carbonyl)piperazine-1- carbonyl]phenyl]imidazole-4- carboxamide; 1:1 2,2,2-trifluoroacetic acid |
| 109 | 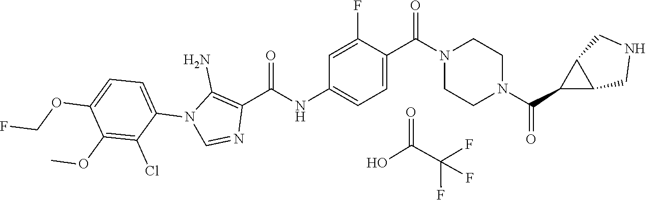 | 5-amino-N-[4-[4-[(1S,5R,6r)-3- azabicyclo[3.1.0]hexane-6- carbonyl]piperazine-1-carbonyl]-3- fluoro-phenyl]-1-[2-chloro-4- (fluoromethoxy)-3- methoxyphenyl]imidazole-4- carboxamide; 1:1 2,2,2-trifluoroacetic acid |
| 110 | 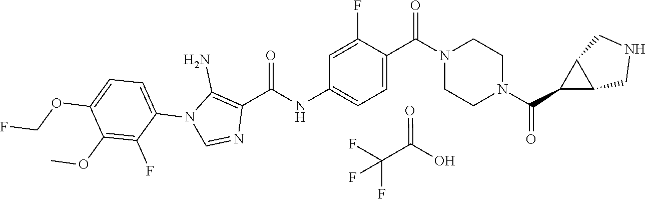 | N-(4-(4-((1R,5S,6r)-3-azabicyclo [3.1.0] hexane-6-carbonyl) piperazine-1- carbonyl)-3-fluorophenyl)-5-amino-1-(2- fluoro-4-(fluoromethoxy)-3- methoxyphenyl)-1H-imidazole-4- carboxamide, 1:1 2,2,2-trifluoroacetate |
| 111 | 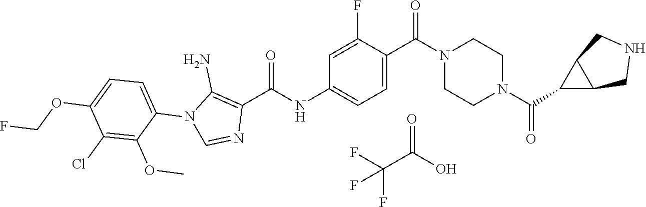 | N-(4-(4-((1S,5R,6S6s)-3- azabicyclo[3.1.0]hexane-6- carbonyl)piperazine-1-carbonyl)-3- fluorophenyl)-5-amino-1-(3-chloro-4- (fluoromethoxy)-2-methoxyphenyl)-1H- imidazole-4-carboxamide, 1:1 2,2,2- trifluoroacetate acid |
| 112 | 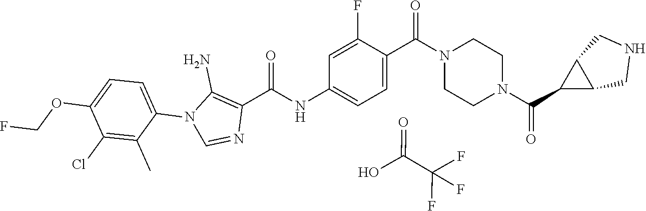 | N-(4-(4-((1R,5S,6r)-3- azabicyclo[3.1.0]hexane-6- carbonyl)piperazine-1-carbonyl)-3- fluorophenyl)-5-amino-1-(3-chloro-4- (fluoromethoxy)-2-methylphenyl)-1H- imidazole-4-carboxamide 1:1 2,2,2- trifluoroacetate acid |
| 113 | 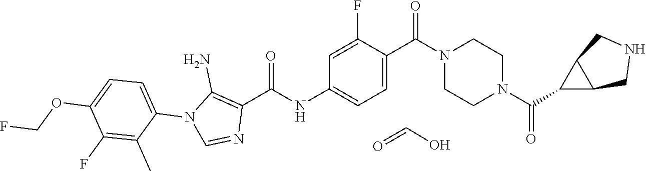 | 5-amino-1-[3-fluoro-4-(fluoromethoxy)- 2-methylphenyl]-N-[3-fluoro-4-[4- [(1R,5S,6r)-3-azabicyclo[3.1.0]hexane- 6-carbonyl]piperazine-1-carbonyl] phenyl]imidazole-4-carboxamide; 1:1 formic acid |
| 114 | 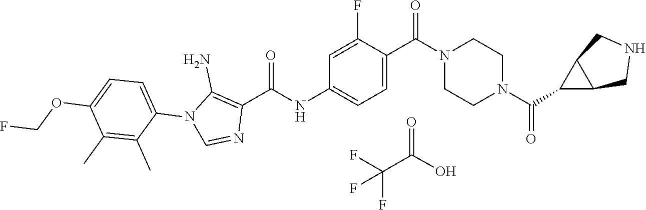 | N-(4-(4-((1R,5S,6r)-3-azabicyclo [3.1.0] hexane-6-carbonyl) piperazine-1- carbonyl)-3-fluorophenyl)-5-amino-1-(4- (fluoromethoxy)-2,3-dimethylphenyl)- 1H-imidazole-4-carboxamide 2,2,2- trifluoroacetate; 1:1 trifluoroacetate |
| 115 | 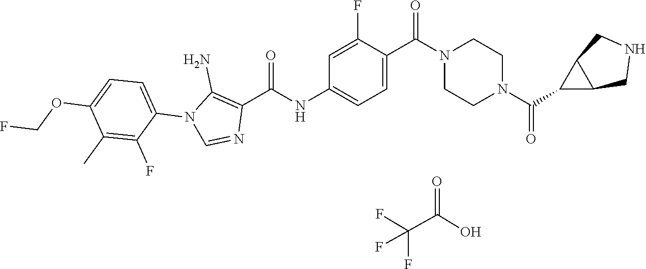 [SMILES: Cc1c(OCF)ccc(-n2cnc(C(=O)Nc3ccc(C(=O)N4CCN(C(=O)[C@H]5[C@@H]6CNC[C@@H]65)CC4)c(F)c3)c2N)c1F ; O=C(O)C(F)(F)F] | N-(4-(4-((1R,5S,6r)-3- azabicyclo[3.1.0]hexane-6- carbonyl)piperazine-1-carbonyl)-3- fluorophenyl)-5-amino-1-(2-fluoro-4- (fluoromethoxy)-3-methylphenyl)-1H- imidazole-4-carboxamide,6r; 1:1 2,2,2- trifluoroacetate acid |
| 116 | 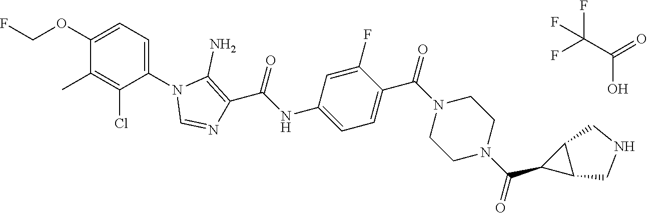 | 5-amino-N-[4-[4-[(1R,5S,6r)-3- azabicyclo[3.1.0]hexane-6- carbonyl]piperazine-1-carbonyl]-3- fluoro-phenyl]-1-[2-chloro-4- (fluoromethoxy)-3-methylphenyl] imidazole-4-carboxamide; 1:1 2,2,2- trifluoroacetic acid |
| 117 | 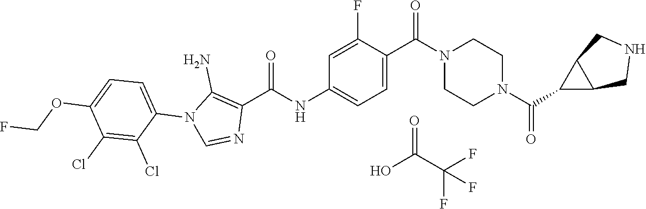 | 5-amino-N-[4-[4-[(1R,5S,6r)-3- azabicyclo[3.1.0]hexane-6- carbonyl]piperazine-1-carbonyl]-3- fluoro-phenyl]-1-[2,3-dichloro-4- (fluoromethoxy)phenyl]imidazole-4- carboxamide; 1:1 2,2,2-trifluoroacetic acid |
| 118 | 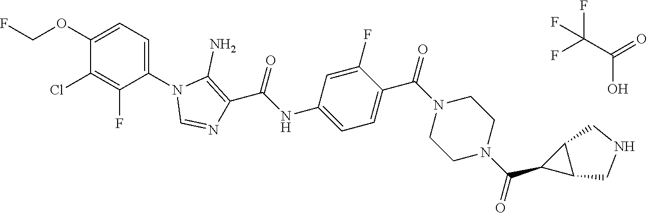 | 5-amino-N-[4-[4-[(1R,5S,6r)-3- azabicyclo[3.1.0]hexane-6- carbonyl]piperazine-1-carbonyl]-3- fluoro-phenyl]-1-[3-chloro-2-fluoro-4- (fluoromethoxy)phenyl]imidazole-4- carboxamide; 1:1 2,2,2-trifluoroacetic acid |
| 119 | 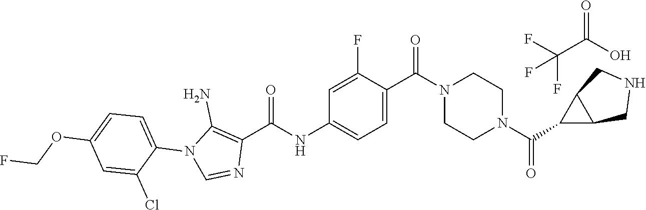 | 5-amino-N-[4-[4-[(1R,5S,6r)-3- azabicyclo[3.1.0]hexane-6- carbonyl]piperazine-1-carbonyl]-3- fluoro-phenyl]-1-[2-chloro-4- (fluoromethoxy)phenyl]imidazole-4- carboxamide; 1:1 2,2,2-trifluoroacetic acid |
| 120 | 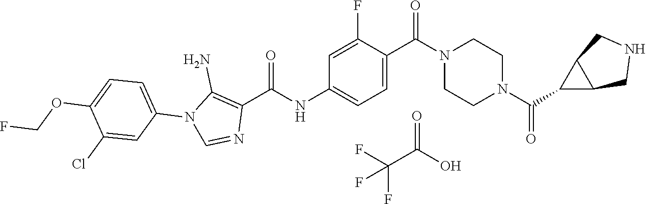 | N-(4-(4-(1R,5S,6r)-3- azabicyclo[3.1.0]hexane-6- carbonyl)piperazine-1-carbonyl)-3- fluorophenyl)-5-amino-1-(3-chloro-4- (fluoromethoxy)phenyl)-1H-imidazole-4- carboxamide; 1:1 trifluoroacetate |
| 121 | 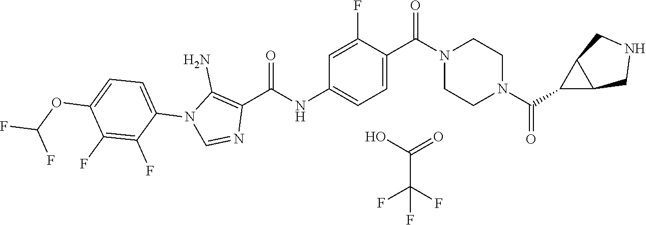 | 5-amino-1-[4-(difluoromethoxy)-2,3- difluoro-phenyl]-N-[3-fluoro-4-[4- [(1R,5S,6r)-3-azabicyclo[3.1.0]hexane- 6-carbonyl]piperazine-1- carbonyl]phenyl]imidazole-4- carboxamide; 1:1 2,2,2-trifluoroacetic acid |
| 122 | 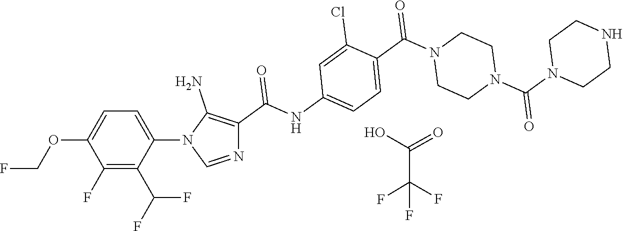 | 5-amino-N-[3-chloro-4-[4-(piperazine-1- carbonyl)piperazine-1-carbonyl]phenyl]- 1-[2-(difluoromethyl)-3-fluoro-4- (fluoromethoxy)phenyl]imidazole-4- carboxamide; 1:1 2,2,2-trifluoroacetic acid |
| 123 | 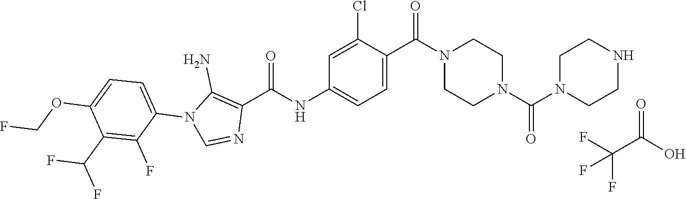 | 5-amino-N-(3-chloro-4-(4-(piperazine-1- carbonyl)piperazine-1-carbonyl)phenyl)- 1-(3-(difluoromethyl)-2-fluoro-4- (fluoromethoxy)phenyl)-1H-imidazole-4- carboxamide; 1:1 2,2,2-trifluoroacetate acid |
| 124 | 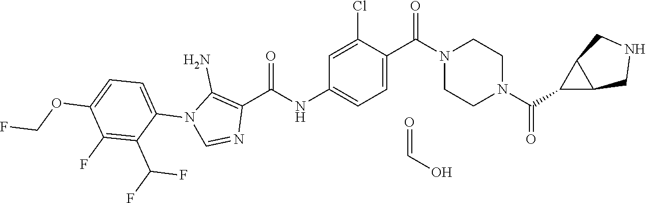 | 5-amino-N-[3-chloro-4-[4-[(1R,5S,6r)-3- azabicyclo[3.1.0]hexane-6- carbonyl]piperazine-1-carbonyl]phenyl]- 1-[2-(difluoromethyl)-3-fluoro-4- (fluoromethoxy)phenyl]imidazole-4- carboxamide; 1:1 formic acid |
| 125 | 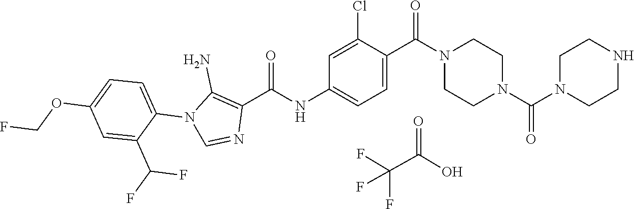 | 5-amino-N-(3-chloro-4-(4-(piperazine-1- carbonyl) piperazine-1-carbonyl) phenyl)-1-(2-(difluoromethyl)-4- (fluoromethoxy) phenyl)-1H-imidazole- 4-carboxamide; 1:1 2,2,2- trifluoroacetate |
| 126 | 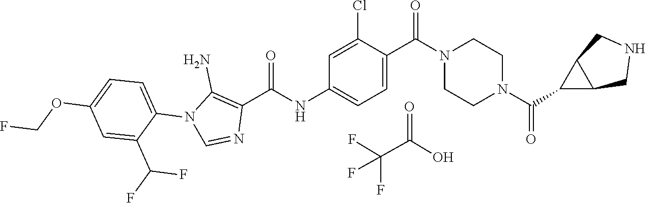 | N-(4-(4-((1R,5S,6r)-3- azabicyclo[3.1.0]hexane-6- carbonyl)piperazine-1-carbonyl)-3- chlorophenyl)-5-amino-1-(2- (difluoromethyl)-4-(fluoromethoxy) phenyl)-1H-imidazole-4-carboxamide 1:1 2,2,2-trifluoroacetate |
| 127 | 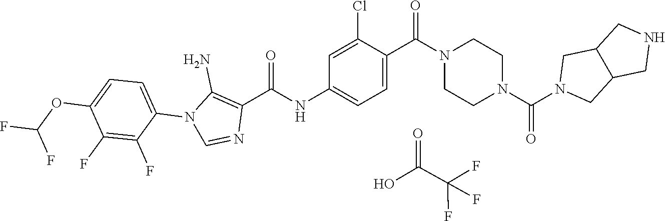 [SMILES: Nc1c(C(=O)Nc2ccc(C(=O)N3CCN(C(=O)N4CC5CNCC5C4)CC3)c(Cl)c2)ncn1-c1ccc(OC(F)F)c(F)c1F ; O=C(O)C(F)(F)F] | N-[4-[4-(2,3,3a,4,6,6a-hexahydro-1H- pyrrolo[3,4-c]pyrrole-5- carbonyl)piperazine-1-carbonyl]-3- chloro-phenyl]-5-amino-1-[4- (difluoromethoxy)-2,3-difluoro- phenyl]imidazole-4-carboxamide; 1:1 2,2,2-trifluoroacetic acid |
| 128 | 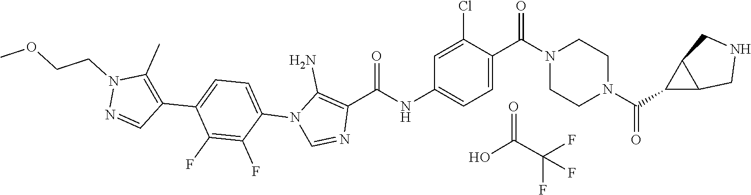 | 5-amino-N-[4-[4-[(1R,5S,6r)-3- azabicyclo[3.1.0]hexane-6- carbonyl]piperazine-1-carbonyl]-3- chloro-phenyl]-1-[2,3-difluoro-4-[1-(2- methoxyethyl)-5-methyl-pyrazol-4- yl]phenyl] imidazole-4-carboxamide; 1:1 2,2,2-trifluoroacetic acid |
| 129 | 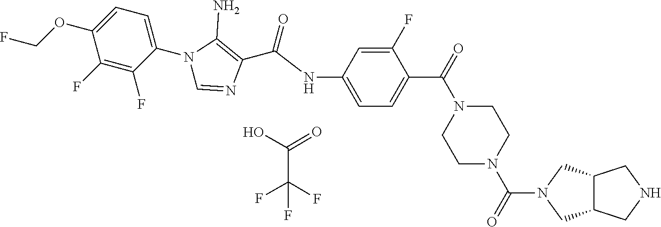 [SMILES: Nc1c(C(=O)Nc2ccc(C(=O)N3CCN(C(=O)N4C[C@H]5CNC[C@H]5C4)CC3)c(F)c2)ncn1-c1ccc(OCF)c(F)c1F ; O=C(O)C(F)(F)F] | 5-amino-1-[2,3-difluoro-4- (fluoromethoxy)phenyl]-N-[3-fluoro-4-[4- [(3aS,6aR)-2,3,3a,4,6,6a-hexahydro- 1H-pyrrolo[3,4-c]pyrrole-5- carbonyl]piperazine-1- carbonyl]phenyl]imidazole-4- carboxamide; 1:1 2,2,2-trifluoroacetic acid |
| 130 | 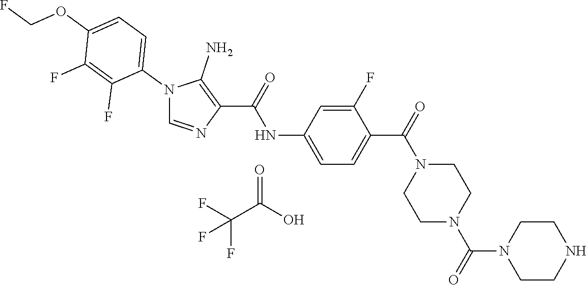 | 5-amino-1-[2,3-difluoro-4- (fluoromethoxy)phenyl]-N-[3-fluoro-4-[4- (piperazine-1-carbonyl)piperazine-1- carbonyl]phenyl]imidazole-4- carboxamide; 1:1 2,2,2-trifluoroacetic acid |
| 131 | 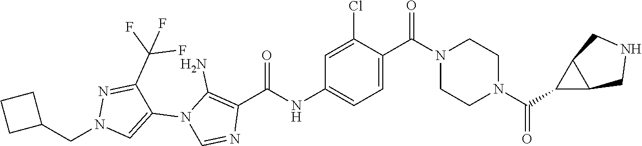 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2CCC2)nc1C(F)(F)F] | 5-amino-N-[4-[4-[(1R,5S,6r)-3- azabicyclo[3.1.0]hexane-6- carbonyl]piperazine-1-carbonyl]-3- chloro-phenyl]-1-[1-(cyclobutylmethyl)- 3-(trifluoromethyl)pyrazol-4-yl]imidazole- 4-carboxamide |
| 132 | 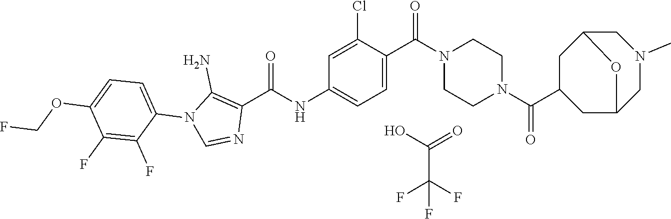 | 5-amino-N-[3-chloro-4-[4-(7-methyl-9- oxa-3,7-diazabicyclo[3.3.1]nonane-3- carbonyl) piperazine-1-carbonyl]phenyl]- 1-[2,3-difluoro-4-(fluoromethoxy)phenyl] imidazole-4-carboxamide; 1:1 2,2,2- trifluoroacetic acid |
| 133 | 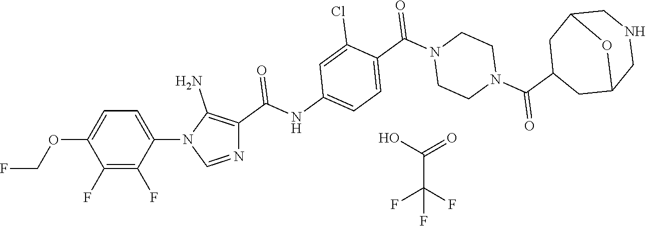 | 5-amino-N-[3-chloro-4-[4-(9-oxa-3,7- diazabicyclo[3.3.1]nonane-3- carbonyl)piperazine-1-carbonyl]phenyl]- 1-[2,3-difluoro-4-(fluoromethoxy)phenyl] imidazole-4-carboxamide; 1:1 2,2,2- trifluoroacetic acid |
| 134 | 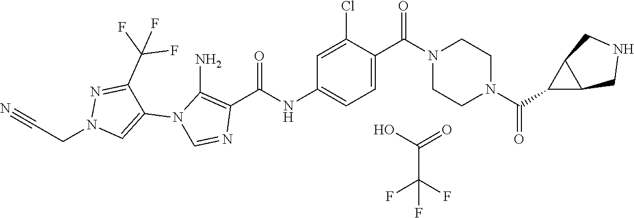 | N-(4-(4-((1R,5S,6r)-3- azabicyclo[3.1.0]hexane-6- carbonyl)piperazine-1-carbonyl)-3- chlorophenyl)-5-amino-1-(1- (cyanomethyl)-3-(trifluoromethyl)-1H- pyrazol-4-yl)-1H-imidazole-4- carboxamide; 1:1 2,2,2-trifluoroacetic acid |
| 135 | 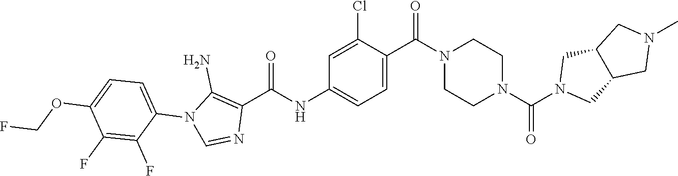 [SMILES: CN1C[C@@H]2CN(C(=O)N3CCN(C(=O)c4ccc(NC(=O)c5ncn(-c6ccc(OCF)c(F)c6F)c5N)cc4Cl)CC3)C[C@@H]2C1] | N-[4-[4-[(3aS,6aR)-2-methyl- 1,3,3a,4,6,6a-hexahydropyrrolo[3,4- c]pyrrole-5-carbonyl]piperazine-1- carbonyl]-3-chloro-phenyl]-5-amino-1- [2,3-difluoro-4- (fluoromethoxy)phenyl]imidazole-4- carboxamide |
| 136 | 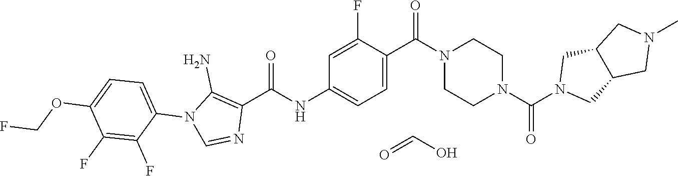 [SMILES: CN1C[C@@H]2CN(C(=O)N3CCN(C(=O)c4ccc(NC(=O)c5ncn(-c6ccc(OCF)c(F)c6F)c5N)cc4F)CC3)C[C@@H]2C1 ; O=CO] | N-[4-[4-[(3aS,6aR)-2-methyl- 1,3,3a,4,6,6a-hexahydropyrrolo[3,4- c]pyrrole-5-carbonyl]piperazine-1- carbonyl]-3-fluoro-phenyl]-5-amino-1- [2,3-difluoro-4- (fluoromethoxy)phenyl]imidazole-4- carboxamide; 1:1 formic acid |
| 137 | 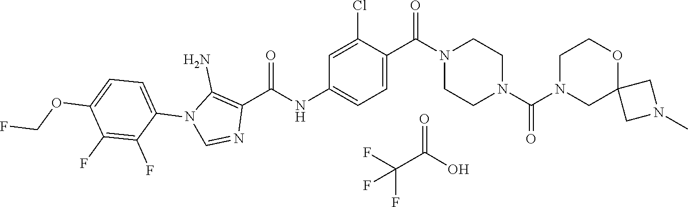 | 5-amino-N-(3-chloro-4-(4-(2-methyl-5- oxa-2,8-diazaspiro [3.5] nonane-8- carbonyl) piperazine-1-carbonyl) phenyl)-1-(2,3-difluoro-4- (fluoromethoxy) phenyl)-1H-imidazole- 4-carboxamide; 1:1 2,2,2- trifluoroacetate |
| 138 | 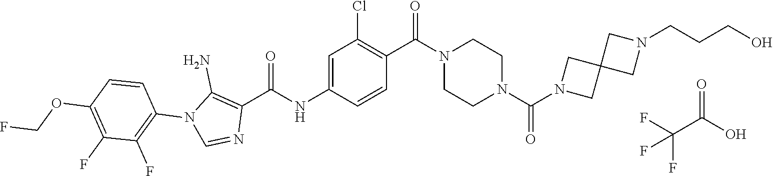 | 5-amino-N-[3-chloro-4-[4-[6-(3- hydroxypropyl)-2,6- diazaspiro[3.3]heptane-2- carbonyl]piperazine-1-carbonyl]phenyl]- 1-[2,3-difluoro-4- (fluoromethoxy)phenyl]imidazole-4- carboxamide; 1:1 2,2,2-trifluoroacetic acid |
| 139 | 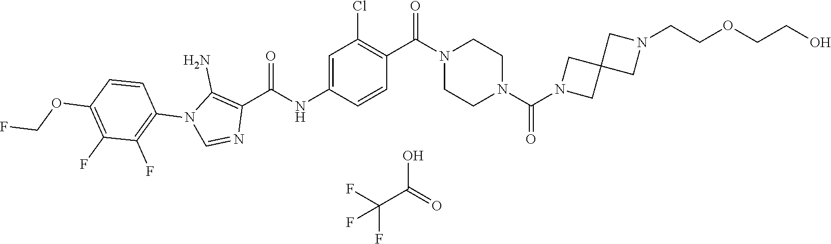 [SMILES: Nc1c(C(=O)Nc2ccc(C(=O)N3CCN(C(=O)N4CC5(CN(CCOCCO)C5)C4)CC3)c(Cl)c2)ncn1-c1ccc(OCF)c(F)c1F ; O=C(O)C(F)(F)F] | 5-amino-N-(3-chloro-4-(4-(6-(2-(2- hydroxyethoxy)ethyl)-2,6- diazaspiro[3.3]heptane-2- carbonyl)piperazine-1-carbonyl)phenyl)- 1-(2,3-difluoro-4- (fluoromethoxy)phenyl)-1H-imidazole-4- carboxamide; 1:1 2,2,2-trifluoroacetic acid |
| 140 | 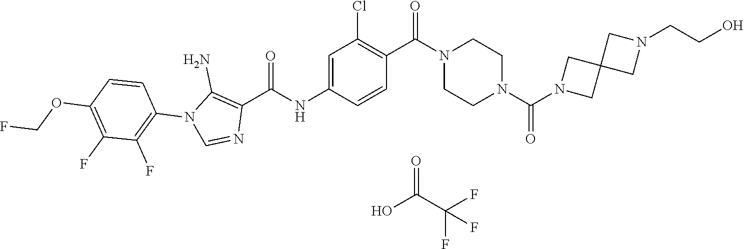 [SMILES: Nc1c(C(=O)Nc2ccc(C(=O)N3CCN(C(=O)N4CC5(CN(CCO)C5)C4)CC3)c(Cl)c2)ncn1-c1ccc(OCF)c(F)c1F ; O=C(O)C(F)(F)F] | 5-amino-N-[3-chloro-4-[4-[6-(2- hydroxyethyl)-2,6- diazaspiro[3.3]heptane-2- carbonyl]piperazine-1-carbonyl]phenyl]- 1-[2,3-difluoro-4-(fluoromethoxy)phenyl] imidazole-4-carboxamide; 1:1 2,2,2- trifluoroacetic acid |
| 141 | 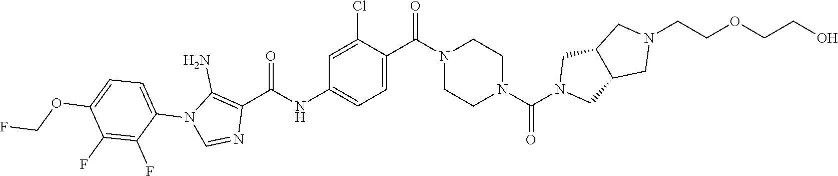 [SMILES: Nc1c(C(=O)Nc2ccc(C(=O)N3CCN(C(=O)N4C[C@H]5CN(CCOCCO)C[C@H]5C4)CC3)c(Cl)c2)ncn1-c1ccc(OCF)c(F)c1F] | N-[4-[4-[(3aS,6aR)-2-[2-(2- hydroxyethoxy)ethyl]-1,3,3a,4,6,6a- hexahydropyrrolo[3,4-c]pyrrole-5- carbonyl]piperazine-1-carbonyl]-3- chloro-phenyl]-5-amino-1-[2,3-difluoro- 4-(fluoromethoxy)phenyl]imidazole-4- carboxamide |
| 142 | 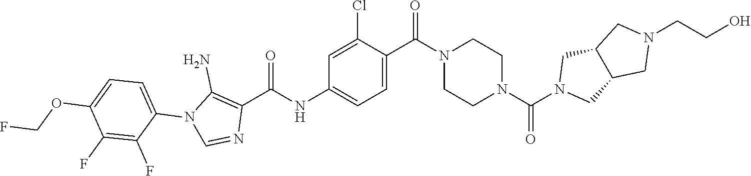 [SMILES: Nc1c(C(=O)Nc2ccc(C(=O)N3CCN(C(=O)N4C[C@H]5CN(CCO)C[C@H]5C4)CC3)c(Cl)c2)ncn1-c1ccc(OCF)c(F)c1F] | N-[4-[4-[(3aS,6aR)-2-(2-hydroxyethyl)- 1,3,3a,4,6,6a-hexahydropyrrolo[3,4- c]pyrrole-5-carbonyl]piperazine-1- carbonyl]-3-chloro-phenyl]-5-amino-1- [2,3-difluoro-4- (fluoromethoxy)phenyl]imidazole-4- carboxamide |
| 143 | 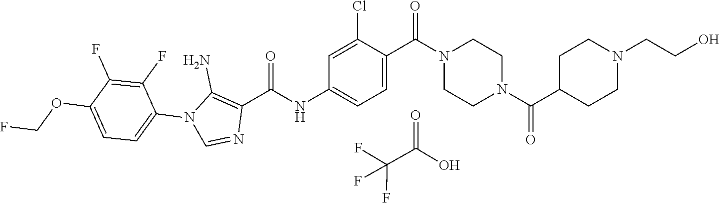 | 5-amino-N-[3-chloro-4-[4-[1-(2- hydroxyethyl) piperidine-4-carbonyl] piperazine-1-carbonyl] phenyl]-1-[2,3- difluoro-4-(fluoromethoxy) phenyl] imidazole-4-carboxamide; 1:1 2,2,2- trifluoroacetic acid |
| 144 | 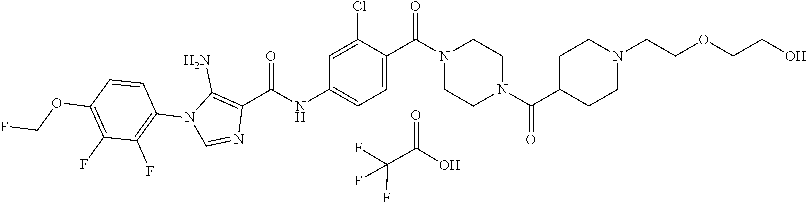 | 5-amino-N-[3-chloro-4-[4-[1-[2-(2- hydroxyethoxy) ethyl] piperidine-4- carbonyl] piperazine-1-carbonyl] phenyl]-1-[2,3-difluoro-4- (fluoromethoxy) phenyl] imidazole-4- carboxamide; 1:1 2,2,2-trifluoroacetic acid |
| 145 | 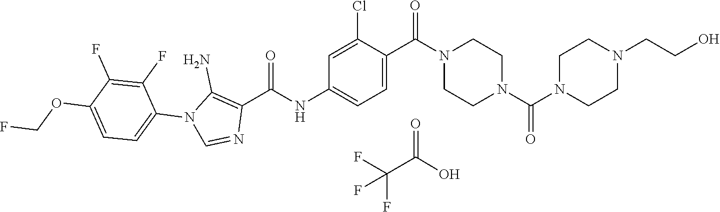 | 5-amino-N-[3-chloro-4-[4-[4-(2- hydroxyethyl) piperazine-1-carbonyl] piperazine-1-carbonyl] phenyl]-1-[2,3- difluoro-4-(fluoromethoxy) phenyl] imidazole-4-carboxamide; 1:1 2,2,2- trifluoroacetic acid |
| 146 | 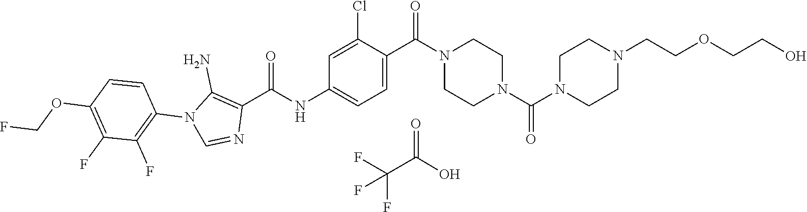 | 5-amino-N-[3-chloro-4-[4-[4-[2-(2- hydroxyethoxy) ethyl] piperazine-1- carbonyl] piperazine-1-carbonyl] phenyl]-1-[2,3-difluoro-4- (fluoromethoxy) phenyl] imidazole-4- carboxamide; 1:1 2,2,2-trifluoroacetic acid |
| 147 | 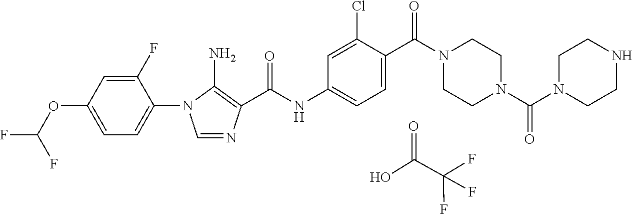 [SMILES: Nc1c(C(=O)Nc2ccc(C(=O)N3CCN(C(=O)N4CCNCC4)CC3)c(Cl)c2)ncn1-c1ccc(OC(F)F)cc1F ; O=C(O)C(F)(F)F] | 5-amino-N-[3-chloro-4-[4-(piperazine-1- carbonyl)piperazine-1-carbonyl]phenyl]- 1-[4-(difluoromethoxy)-2-fluoro- phenyl]imidazole-4-carboxamide; 1:1 2,2,2-trifluoroacetic acid |
| 148 | 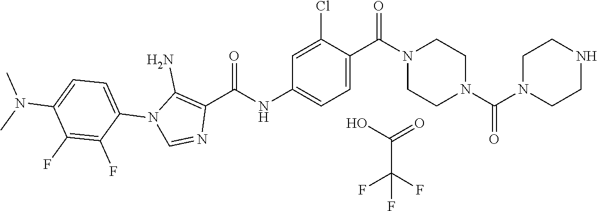 | 5-amino-N-[3-chloro-4-[4-(piperazine-1- carbonyl)piperazine-1-carbonyl]phenyl]- 1-[4-(dimethylamino)-2,3-difluoro- phenyl]imidazole-4-carboxamide; 1;1 2,2,2-trifluoroacetate acid |
| 149 | 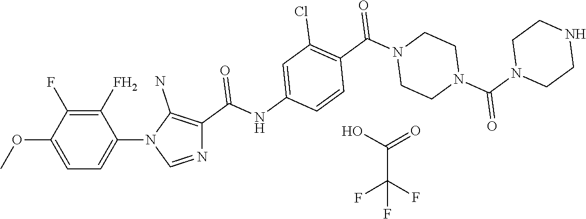 | 5-amino-N-[3-chloro-4-[4-(piperazine-1- carbonyl)piperazine-1-carbonyl]phenyl]- 1-(2,3-difluoro-4- methoxyphenyl)imidazole-4- carboxamide; 1:1 2,2,2-trifluoroacetic acid |
| 150 | 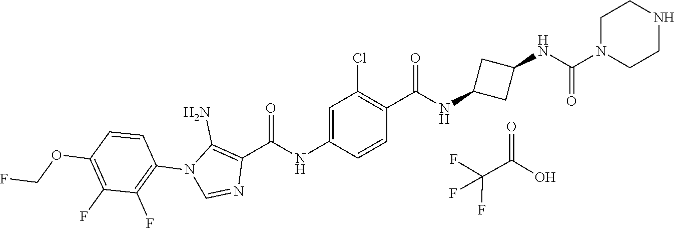 | N-[(1s,3s)-3-[4-[[5-amino-1-[2,3- difluoro-4-(fluoromethoxy) phenyl] imidazole-4-carbonyl]amino]-2- chlorobenzoyl]amino]cyclobutyl] piperazine-1-carboxamide; 1:1 2,2,2- trifluoroacetic acid |
| 151 | 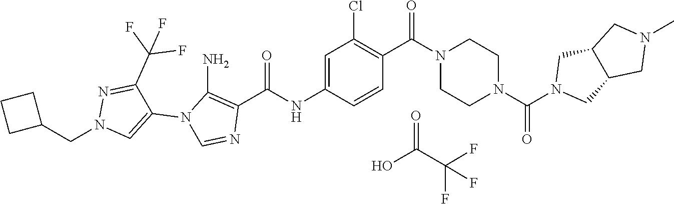 | N-[4-[4-[(3aS,6aR)-2-methyl- 1,3,3a,4,6,6a-hexahydropyrrolo[3,4- c]pyrrole-5-carbonyl]piperazine-1- carbonyl]-3-chloro-phenyl]-5-amino-1- [1-(cyclobutylmethyl)-3- (trifluoromethyl)pyrazol-4-yl]imidazole-4- carboxamide; 1:1 2,2,2-trifluoroacetic acid |
| 152 | 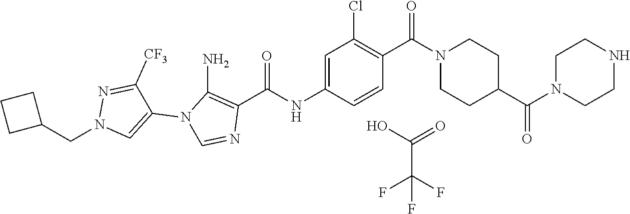 | 5-amino-N-[3-chloro-4-[4-(piperazine-1- carbonyl)piperidine-1-carbonyl]phenyl]- 1-[1-(cyclobutylmethyl)-3- (trifluoromethyl)pyrazol-4-yl]imidazole-4- carboxamide; 1:1 2,2,2-trifluoroacetic acid |
| 153 | 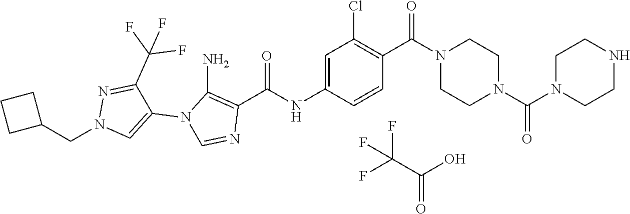 | 5-amino-N-[3-chloro-4-[4-(piperazine-1- carbonyl)piperazine-1-carbonyl]phenyl]- 1-[1-(cyclobutylmethyl)-3- (trifluoromethyl)pyrazol-4-yl]imidazole-4- carboxamide; 1:1 2,2,2-trifluoroacetic acid |
| 154 | 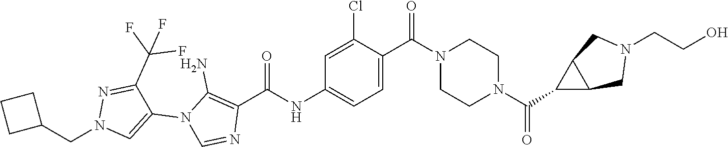 [SMILES: Nc1c(C(=O)Nc2ccc(C(=O)N3CCN(C(=O)[C@H]4[C@@H]5CN(CCO)C[C@@H]54)CC3)c(Cl)c2)ncn1-c1cn(CC2CCC2)nc1C(F)(F)F] | 5-amino-N-[3-chloro-4-[4-[(1R,5S,6r)-3- (2-hydroxyethyl)-3- azabicyclo[3.1.0]hexane-6- carbonyl]piperazine-1-carbonyl]phenyl]- 1-[1-(cyclobutylmethyl)-3- (trifluoromethyl)pyrazol-4-yl]imidazole-4- carboxamide |
| 155 | 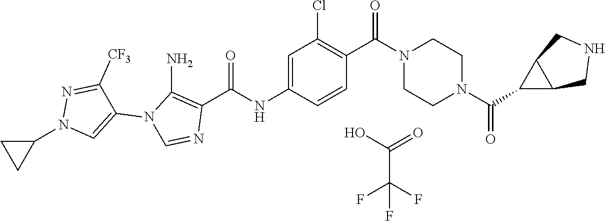 | 5-amino-N-[3-chloro-4-[4-[(1S,5R,6r)-3- azabicyclo[3.1.0]hexane-6- carbonyl]piperazine-1-carbonyl]phenyl]- 1-[1-cyclopropyl-3- (trifluoromethyl)pyrazol-4-yl]imidazole-4- carboxamide; 1:1 2,2,2-trifluoroacetic acid |
| 156 | 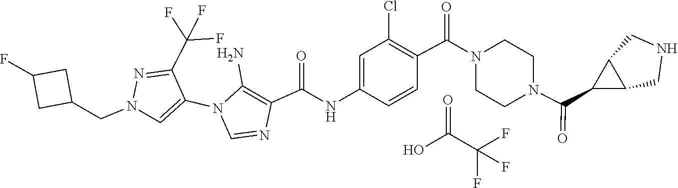 | 5-amino-N-[4-[4-[(1S,5R,6r)-3- azabicyclo[3.1.0]hexane-6- carbonyl]piperazine-1-carbonyl]-3- chloro-phenyl]-1-[1-[(3- fluorocyclobutyl)methyl]-3- (trifluoromethyl)pyrazol-4-yl]imidazole-4- carboxamide; 1:1 2,2,2-trifluoroacetic acid |
| 157 | 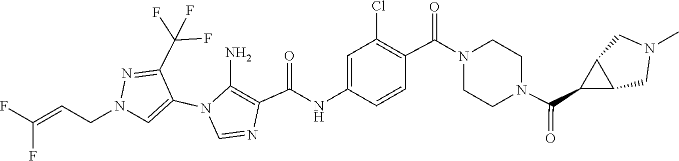 [SMILES: CN1C[C@@H]2[C@H](C1)[C@H]2C(=O)N1CCN(C(=O)c2ccc(NC(=O)c3ncn(-c4cn(CC=C(F)F)nc4C(F)(F)F)c3N)cc2Cl)CC1] | 5-amino-N-[3-chloro-4-[4-[(1S,5R,6r)-3- methyl-3-azabicyclo[3.1.0]hexane-6- carbonyl]piperazine-1-carbonyl]phenyl]- 1-[1-(3,3-difluoroallyl)-3- (trifluoromethyl)pyrazol-4-yl]imidazole-4- carboxamide |
| 158 | 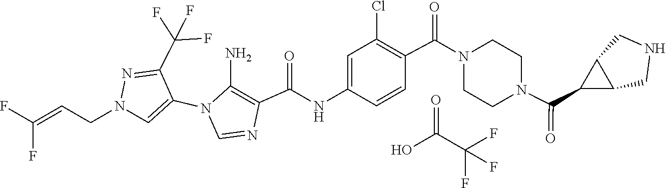 | 5-amino-N-[4-[4-[(1S,5R,6r)-3- azabicyclo[3.1.0]hexane-6- carbonyl]piperazine-1-carbonyl]-3- chloro-phenyl]-1-[1-(3,3-difluoroallyl)-3- (trifluoromethyl)pyrazol-4-yl]imidazole-4- carboxamide; 1:1 2,2,2-trifluoroacetic acid |
| 159 | 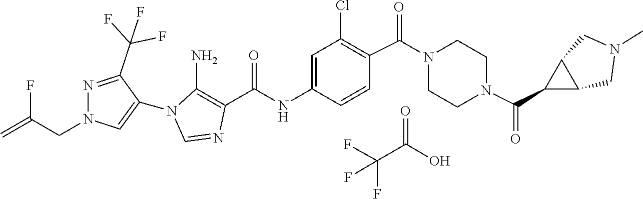 | 5-amino-N-[3-chloro-4-[4-[(1S,5R,6r)-3- methyl-3-azabicyclo[3.1.0]hexane-6- carbonyl]piperazine-1-carbonyl]phenyl]- 1-[1-(2-fluoroallyl)-3- (trifluoromethyl)pyrazol-4-yl]imidazole-4- carboxamide; 1:1 2,2,2-trifluoroacetic acid |
| 160 | 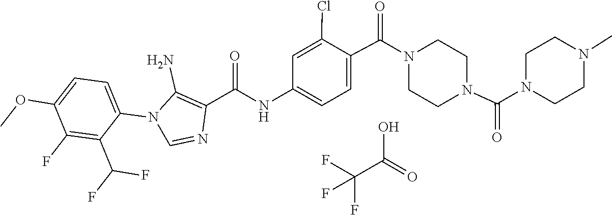 | 5-amino-N-[3-chloro-4-[4-(4- methylpiperazine-1-carbonyl)piperazine- 1-carbonyl]phenyl]-1-[2-(difluoromethyl)- 3-fluoro-4-methoxyphenyl]imidazole-4- carboxamide; 1:1 2,2,2-trifluoroacetic acid |
| 161 | 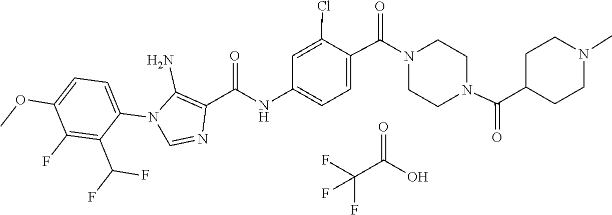 | 5-amino-N-[3-chloro-4-[4-(1- methylpiperidine-4-carbonyl) piperazine- 1-carbonyl] phenyl]-1-[2- (difluoromethyl)-3-fluoro-4- methoxyphenyl] imidazole-4- carboxamide; 1:1 2,2,2-trifluoroacetic acid |
| 162 | 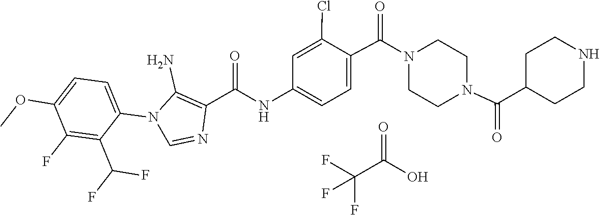 | 5-amino-N-[3-chloro-4-[4-(piperidine-4- carbonyl) piperazine-1-carbonyl] phenyl]-1-[2-(difluoromethyl)-3-fluoro-4- methoxyphenyl] imidazole-4- carboxamide; 1:1 2,2,2-trifluoroacetic acid |
| 163 | 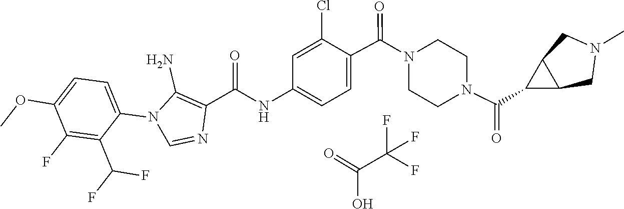 | 5-amino-N-(3-chloro-4-(4-((1R,5S,6r)-3- methyl-3-azabicyclo[3.1.0]hexane-6- carbonyl)piperazine-1-carbonyl)phenyl)- 1-(2-(difluoromethyl)-3-fluoro-4- methoxyphenyl)-1H-imidazole-4- carboxamide; 1:1 2,2,2-trifluoroacetate |
| 164 | 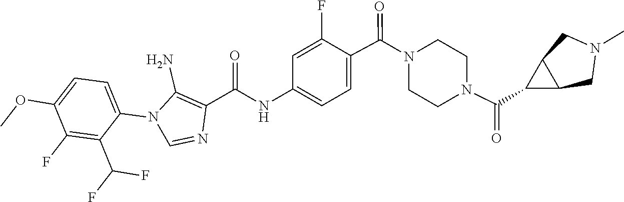 | 5-amino-1-[2-(difluoromethyl)-3-fluoro-4- methoxyphenyl]-N-[3-fluoro-4-[4- [(1R,5S,6r)-3-methyl-3- azabicyclo[3.1.0]hexane-6- carbonyl]piperazine-1- carbonyl]phenyl]imidazole-4- carboxamide |
| 165 | 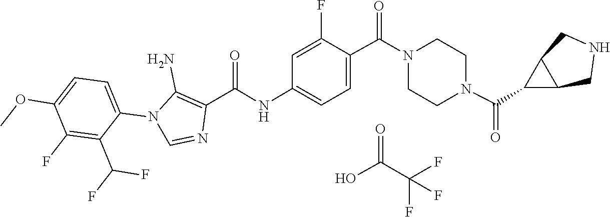 [SMILES: COc1ccc(-n2cnc(C(=O)Nc3ccc(C(=O)N4CCN(C(=O)[C@H]5[C@@H]6CNC[C@@H]65)CC4)c(F)c3)c2N)c(C(F)F)c1F ; O=C(O)C(F)(F)F] | 5-amino-N-[4-[4-[(1R,5S,6r)-3- azabicyclo[3.1.0]hexane-6- carbonyl]piperazine-1-carbonyl]-3- fluoro-phenyl]-1-[2-(difluoromethyl)-3- fluoro-4-methoxyphenyl]imidazole-4- carboxamide; 1:1 2,2,2-trifluoroacetic acid |
| 166 | 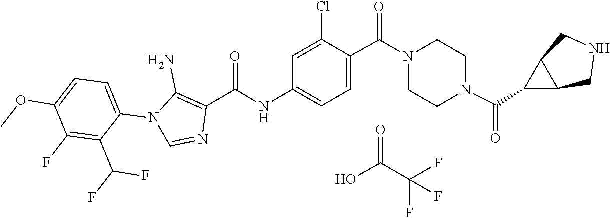 [SMILES: COc1ccc(-n2cnc(C(=O)Nc3ccc(C(=O)N4CCN(C(=O)[C@H]5[C@@H]6CNC[C@@H]65)CC4)c(Cl)c3)c2N)c(C(F)F)c1F ; O=C(O)C(F)(F)F] | N-(4-(4-((1R,5S,6r)-3- azabicyclo[3.1.0]hexane-6- carbonyl)piperazine-1-carbonyl)-3- chlorophenyl)-5-amino-1-(2- (difluoromethyl)-3-fluoro-4- methoxyphenyl)-1H-imidazole-4- carboxamide; 1:1 2,2,2-trifluoroacetate acid |
| 167 | 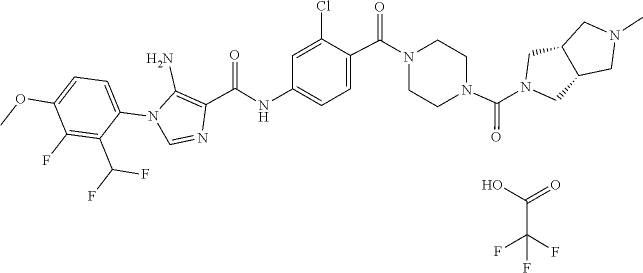 | 5-amino-N-[3-chloro-4-[4-[(3aS,6aR)-2- methyl-1,3,3a,4,6,6a- hexahydropyrrolo[3,4-c]pyrrole-5- carbonyl]piperazine-1-carbonyl]phenyl]- 1-[2-(difluoromethyl)-3-fluoro-4- methoxyphenyl]imidazole-4- carboxamide; 1:1 2,2,2-trifluoroacetic acid |
| 168 | 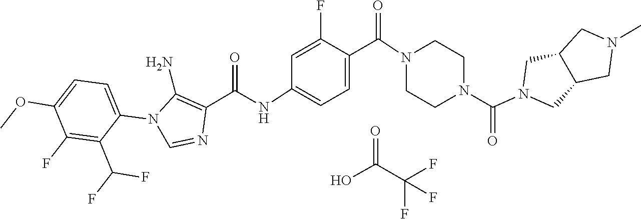 | N-[4-[4-[(3aS,6aR)-2-methyl- 1,3,3a,4,6,6a-hexahydropyrrolo[3,4- c]pyrrole-5-carbonyl]piperazine-1- carbonyl]-3-fluoro-phenyl]-5-amino-1-[2- (difluoromethyl)-3-fluoro-4- methoxyphenyl]imidazole-4- carboxamide; 1:1 2,2,2-trifluoroacetic acid |
| 169 | 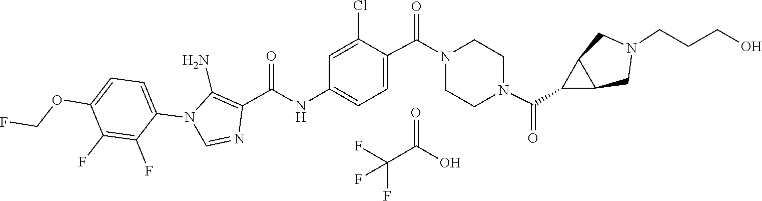 | 5-amino-N-[3-chloro-4-[4-[(1R,5S,6r)-3- (3-hydroxypropyl)-3-azabicyclo [3.1.0]hexane-6-carbonyl]piperazine-1- carbonyl]phenyl]-1-[2,3-difluoro 4- (fluoromethoxy)phenyl]imidazole-4- carboxamide; 1:1 2,2,2-trifluoroacetic acid |
| 170 | 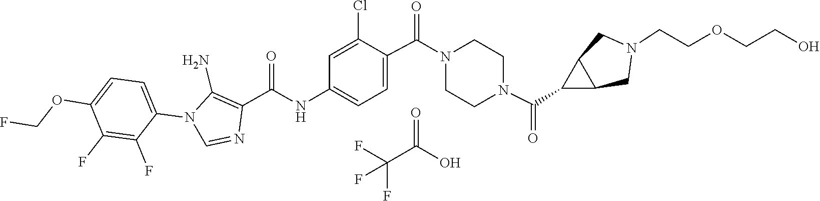 | 5-amino-N-[3-chloro-4-[4-[(1R,5S,6r)-3- [2-(2-hydroxyethoxy) ethyl]-3-azabicyclo [3.1.0] hexane-6-carbonyl] piperazine-1- carbonyl] phenyl]-1-[2,3-difluoro-4- (fluoromethoxy) phenyl] imidazole-4- carboxamide; 1:1 2,2,2-trifluoroacetic acid |
| 171 | 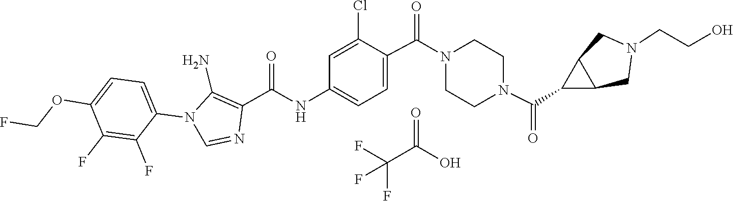 | 5-amino-N-[3-chloro-4-[4-[(1R,5S,6r)-3- (2-hydroxyethyl)-3-azabicyclo [3.1.0] hexane-6-carbonyl] piperazine-1- carbonyl] phenyl]-1-[2,3-difluoro-4- (fluoromethoxy) phenyl] imidazole-4- carboxamide; 1:1 2,2,2-trifluoroacetic acid |
| 172 | 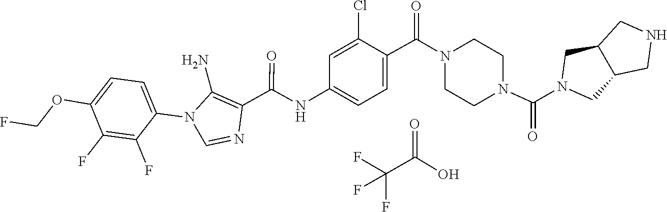 | N-[4-[4-[(3aR,6aR)-2,3,3a,4,6,6a- hexahydro-1H-pyrrolo[3,4-c]pyrrole-5- carbonyl]piperazine-1-carbonyl]-3- chloro-phenyl]-5-amino-1-[2,3-difluoro- 4-(fluoromethoxy)phenyl]imidazole-4- carboxamide; 1:1 2,2,2-trifluoroacetic acid |
| 173 | 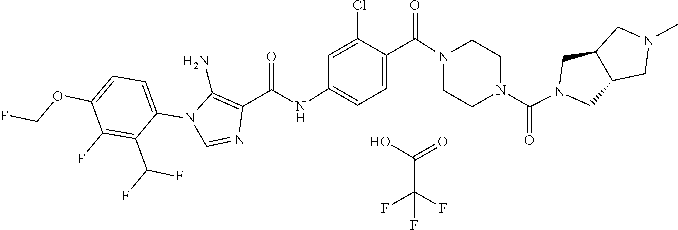 | N-[4-[4-[(3aR,6aR)-2-methyl- 1,3,3a,4,6,6a-hexahydropyrrolo[3,4- c]pyrrole-5-carbonyl]piperazine-1- carbonyl]-3-chloro-phenyl]-5-amino-1- [2-(difluoromethyl)-3-fluoro-4- (fluoromethoxy)phenyl]imidazole-4- carboxamide; 1:1 2,2,2-trifluoroacetic acid |
| 174 | 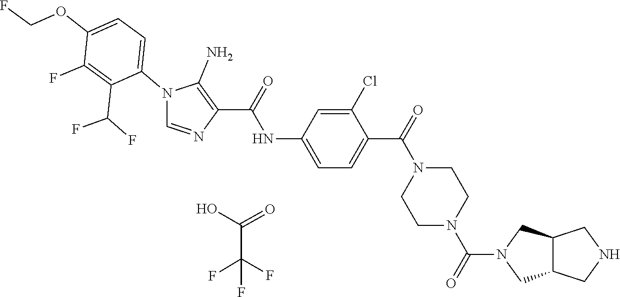 [SMILES: Nc1c(C(=O)Nc2ccc(C(=O)N3CCN(C(=O)N4C[C@H]5CNC[C@@H]5C4)CC3)c(Cl)c2)ncn1-c1ccc(OCF)c(F)c1C(F)F ; O=C(O)C(F)(F)F] | N-[4-[4-[(3aR,6aR)-2,3,3a,4,6,6a- hexahydro-1H-pyrrolo[3,4-c]pyrrole-5- carbonyl] piperazine-1-carbonyl]-3- chloro-phenyl]-5-amino-1-[2- (difluoromethyl)-3-fluoro-4- (fluoromethoxy)phenyl]imidazole-4- carboxamide; 1:1 2,2,2-trifluoroacetic acid |
| 175 | 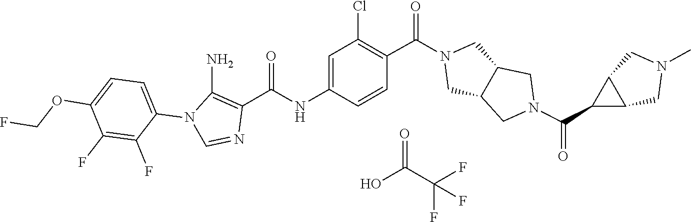 [SMILES: CN1C[C@@H]2[C@H](C1)[C@H]2C(=O)N1C[C@H]2CN(C(=O)c3ccc(NC(=O)c4ncn(-c5ccc(OCF)c(F)c5F)c4N)cc3Cl)C[C@H]2C1 ; O=C(O)C(F)(F)F] | N-[4-[(3aS,6aR)-5-[(1S,5R,6r)-3-methyl- 3-azabicyclo[3.1.0]hexane-6-carbonyl]- 1,3,3a,4,6,6a-hexahydropyrrolo[3,4- c]pyrrole-2-carbonyl]-3-chloro-phenyl]-5- amino-1-[2,3-difluoro-4- (fluoromethoxy)phenyl]imidazole-4- carboxamide; 1:1 2,2,2-trifluoroacetic acid |
| 176 | 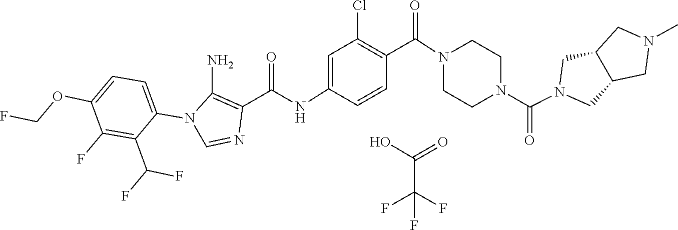 | 5-amino-N-[3-chloro-4-[4-[(3aS,6aR)-2- methyl-1,3,3a,4,6,6a- hexahydropyrrolo[3,4-c]pyrrole-5- carbonyl]piperidine-1-carbonyl]phenyl]- 1-[2-(difluoromethyl)-3-fluoro-4- (fluoromethoxy)phenyl]imidazole-4- carboxamide; 1:1 2,2,2-trifluoroacetic acid |
| 177 | 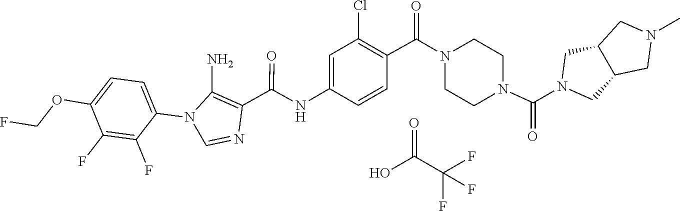 | N-[4-[4-[(3aS,6aR)-2-methyl- 1,3,3a,4,6,6a-hexahydropyrrolo[3,4- c]pyrrole-5-carbonyl]piperidine-1- carbonyl]-3-chloro-phenyl]-5-amino-1- [2,3-difluoro-4- (fluoromethoxy)phenyl]imidazole-4- carboxamide; 1:1 2,2,2-trifluoroacetic acid |
| 178 | 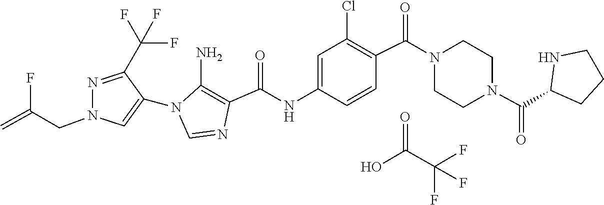 | 5-amino-N-[3-chloro-4-[4-[(2R)- pyrrolidine-2-carbonyl]piperazine-1- carbonyl]phenyl]-1-[1-(2-fluoroallyl)-3- (trifluoromethyl)pyrazol-4-yl]imidazole-4- carboxamide; 2,2,2-trifluoroacetic acid |
| 179 | 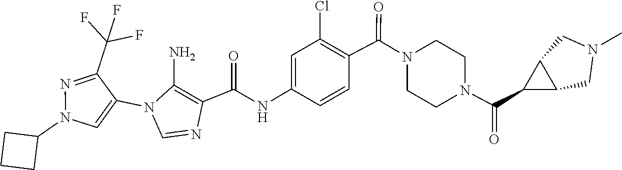 [SMILES: CN1C[C@@H]2[C@H](C1)[C@H]2C(=O)N1CCN(C(=O)c2ccc(NC(=O)c3ncn(-c4cn(C5CCC5)nc4C(F)(F)F)c3N)cc2Cl)CC1] | 5-amino-N-[3-chloro-4-[4-[(1S,5R,6r)-3- methyl-3-azabicyclo[3.1.0]hexane-6- carbonyl]piperazine-1-carbonyl]phenyl]- 1-[1-cyclobutyl-3- (trifluoromethyl)pyrazol-4-yl]imidazole-4- carboxamide |
| 180 | 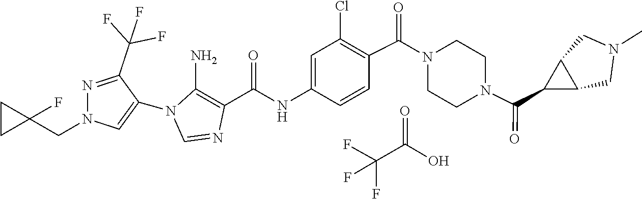 | 5-amino-N-[3-chloro-4-[4-[(1S,5R,6r)-3- methyl-3-azabicyclo[3.1.0]hexane-6- carbonyl]piperazine-1-carbonyl]phenyl]- 1-[1-[(1-fluorocyclopropyl)methyl]-3- (trifluoromethyl)pyrazol-4-yl]imidazole-4- carboxamide; 1:1 2,2,2-trifluoroacetic acid |
| 181 | 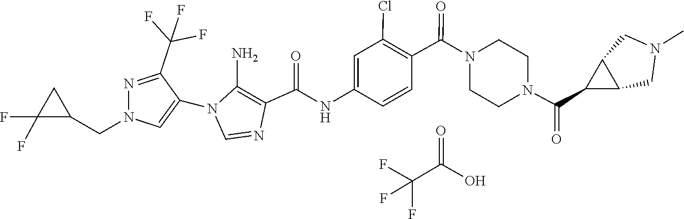 [SMILES: CN1C[C@@H]2[C@H](C1)[C@H]2C(=O)N1CCN(C(=O)c2ccc(NC(=O)c3ncn(-c4cn(CC5CC5(F)F)nc4C(F)(F)F)c3N)cc2Cl)CC1 ; O=C(O)C(F)(F)F] | 5-amino-N-[3-chloro-4-[4-[(1S,5R,6r)-3- methyl-3-azabicyclo[3.1.0]hexane-6- carbonyl]piperazine-1-carbonyl]phenyl]- 1-[1-[(2,2-difluorocyclopropyl)methyl]-3- (trifluoromethyl)pyrazol-4-yl]imidazole-4- carboxamide; 1:1 2,2,2-trifluoroacetic acid |
| 182 | 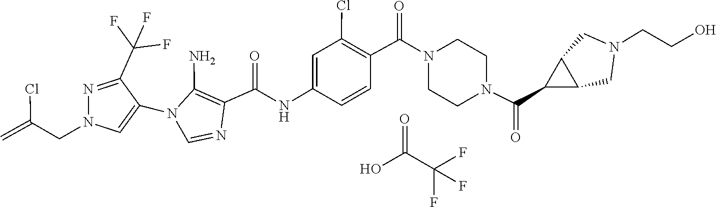 | 5-amino-1-[1-(2-chloroallyl)-3- (trifluoromethyl)pyrazol-4-yl]-N-[3- chloro-4-[4-[(1S,5R,6r)-3-(2- hydroxyethyl)-3- azabicyclo[3.1.0]hexane-6- carbonyl]piperazine-1- carbonyl]phenyl]imidazole-4- carboxamide; 1:1 2,2,2-trifluoroacetic acid |
| 183 | 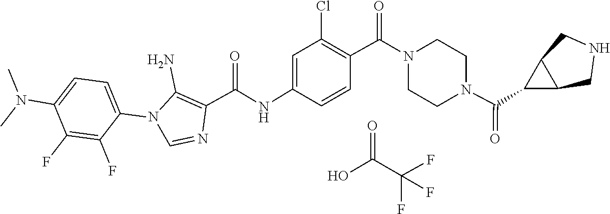 | 5-amino-N-[4-[4-[(1R,5S,6r)-3- azabicyclo[3.1.0]hexane-6- carbonyl]piperazine-1-carbonyl]-3- chloro-phenyl]-1-[4-(dimethylamino)-2,3- difluoro-phenyl]imidazole-4- carboxamide; 1:1 2,2,2-trifluoroacetic acid |
| 184 | 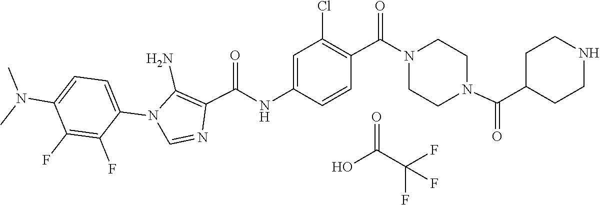 | 5-amino-N-[3-chloro-4-[4-(piperidine-4- carbonyl)piperazine-1-carbonyl]phenyl]- 1-[4-(dimethylamino)-2,3-difluoro- phenyl]imidazole-4-carboxamide; 1:1 2,2,2-trifluoroacetic acid |
| 185 | 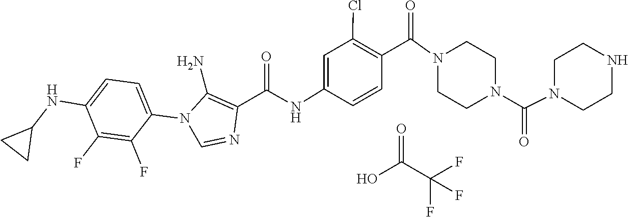 | 5-amino-N-[3-chloro-4-[4-(piperazine-1- carbonyl)piperazine-1-carbonyl]phenyl]- 1-[4-(cyclopropylamino)-2,3- difluorophenyl]imidazole-4- carboxamide; 1:1 2,2,2-trifluoroacetic acid |
| 186 | 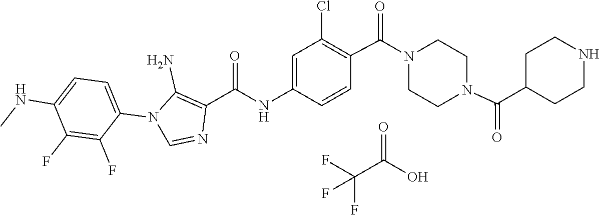 | 5-amino-N-[3-chloro-4-[4-(piperazine-1- carbonyl)piperazine-1-carbonyl]phenyl]- 1-[2,3-difluoro-4-(methylamino)phenyl] imidazole-4-carboxamide; 1:1 2,2,2- trifluoroacetic acid |
| 187 | 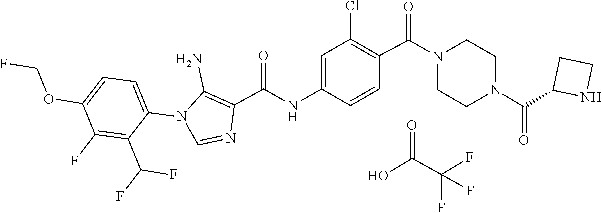 | 5-amino-N-[4-[4-[(2S)-azetidine-2- carbonyl]piperazine-1-carbonyl]-3- chloro-phenyl]-1-[2-(difluoromethyl)-3- fluoro-4- (fluoromethoxy)phenyl]imidazole-4- carboxamide; 1:1 2,2,2-trifluoroacetic acid |
| 188 | 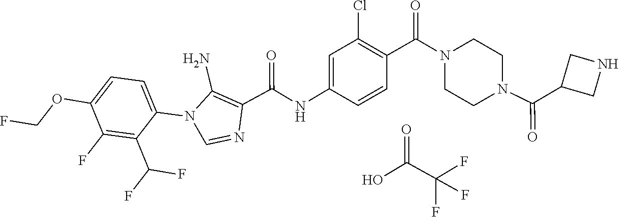 | 5-amino-N-[4-[4-(azetidine-3- carbonyl)piperazine-1-carbonyl]-3- chloro-phenyl]-1-[2-(difluoromethyl)-3- fluoro-4- (fluoromethoxy)phenyl]imidazole-4- carboxamide; 1:1 2,2,2-trifluoroacetic acid |
| 189 | 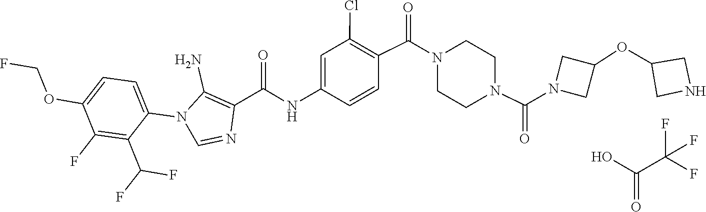 | 5-amino-N-[4-[4-[3-(azetidin-3- yloxy)azetidine-1-carbonyl]piperazine-1- carbonyl]-3-chlorophenyl]-1-[2- (difluoromethyl)-3-fluoro-4- (fluoromethoxy)phenyl]imidazole-4- carboxamide; 1:1 2,2,2-trifluoroacetic acid |
| 190 | 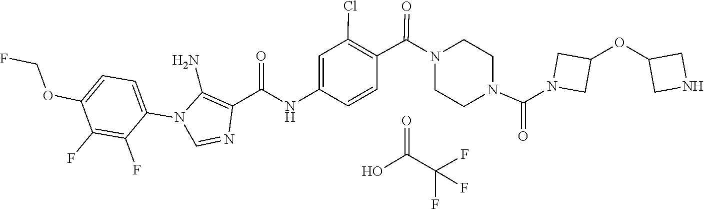 | 5-amino-N-[4-[4-[3-(azetidin-3- yloxy)azetidine-1-carbonyl]piperazine-1- carbonyl]-3-chlorophenyl]-1-[2,3- difluoro-4- (fluoromethoxy)phenyl]imidazole-4- carboxamide; 2,2,2-trifluoroacetic acid |
| 191 | 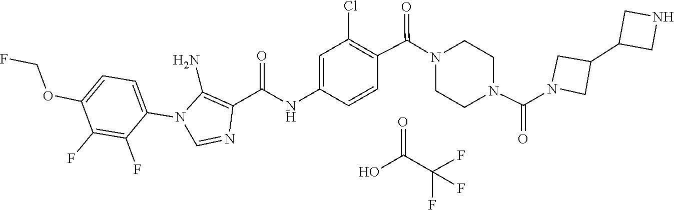 | 5-amino-N-[4-[4-[3-(azetidin-3- yl)azetidine-1-carbonyl]piperazine-1- carbonyl]-3-chlorophenyl]-1-[2,3- difluoro-4- (fluoromethoxy)phenyl]imidazole-4- carboxamide; 1:1 2,2,2-trifluoroacetic acid |
| 192 | 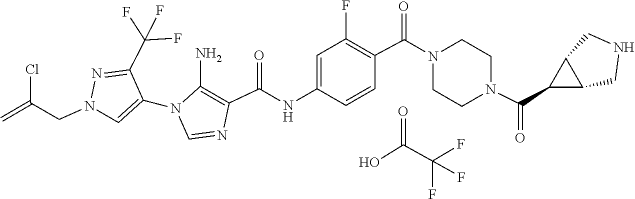 | 5-amino-N-[4-[4-[(1S,5R,6r)-3- azabicyclo[3.1.0]hexane-6- carbonyl]piperazine-1-carbonyl]-3- fluoro-phenyl]-1-[1-(2-chloroallyl)-3- (trifluoromethyl)pyrazol-4-yl]imidazole-4- carboxamide; 1:1 2,2,2-trifluoroacetic acid |
| 193 | 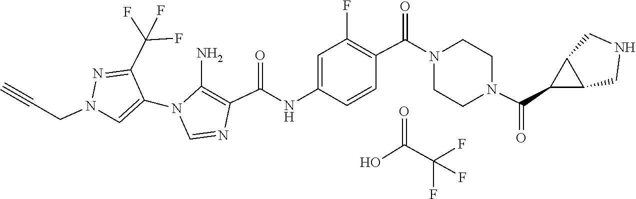 | 5-amino-N-[4-[4-[(1S,5R,6r)-3- azabicyclo[3.1.0]hexane-6- carbonyl]piperazine-1-carbonyl]-3- fluorophenyl]-1-[1-prop-2-ynyl-3- (trifluoromethyl)pyrazol-4-yl]imidazole-4- carboxamide; 1:1 2,2,2-trifluoroacetic acid |
| 194 | 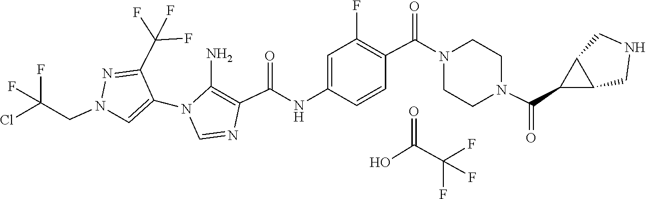 | 5-amino-N-[4-[4-[(1S,5R,6r)-3- azabicyclo[3.1.0]hexane-6- carbonyl]piperazine-1-carbonyl]-3- fluorophenyl]-1-[1-(2-chloro-2,2-difluoro- ethyl)-3-(trifluoromethyl)pyrazol-4- yl]imidazole-4-carboxamide; 1:1 2,2,2- trifluoroacetic acid |
| 195 | 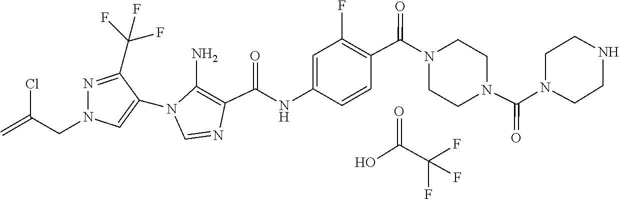 | 5-amino-1-[1-(2-chloroallyl)-3- (trifluoromethyl)pyrazol-4-yl]-N-[3-fluoro- 4-[4-(piperazine-1-carbonyl)piperazine- 1-carbonyl]phenyl]imidazole-4- carboxamide; 1:1 2,2,2-trifluoroacetic acid |
| 196 | 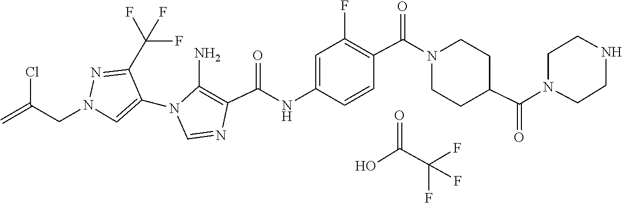 | 5-amino-1-[1-(2-chloroallyl)-3- (trifluoromethyl)pyrazol-4-yl]-N-[3-fluoro- 4-[4-(piperazine-1-carbonyl)piperidine- 1-carbonyl]phenyl]imidazole-4- carboxamide; 2,2,2-trifluoroacetic acid |
| 197 | 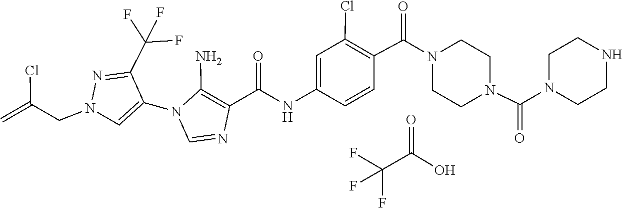 | 5-amino-1-[1-(2-chloroallyl)-3- (trifluoromethyl) pyrazol-4-yl]-N-[3- chloro-4-[4-(piperazine-1- carbonyl)piperazine-1- carbonyl]phenyl]imidazole-4- carboxamide; 1:1 2,2,2-trifluoroacetic acid |
| 198 | 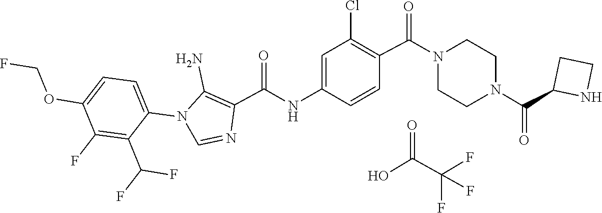 | 5-amino-N-[4-[4-[(2R)-azetidine-2- carbonyl]piperazine-1-carbonyl]-3- chlorophenyl]-1-[2-(difluoromethyl)-3- fluoro-4- (fluoromethoxy)phenyl]imidazole-4- carboxamide; 1:1 2,2,2-trifluoroacetic acid |
| 199 | 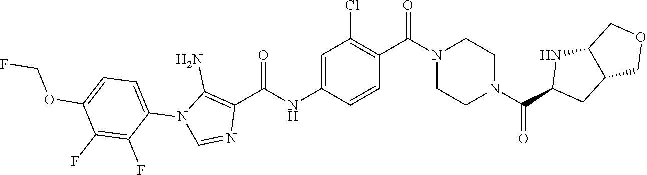 [SMILES: Nc1c(C(=O)Nc2ccc(C(=O)N3CCN(C(=O)[C@@H]4C[C@H]5COC[C@H]5N4)CC3)c(Cl)c2)ncn1-c1ccc(OCF)c(F)c1F] | N-[4-[4-[(2S,3aR,6aS)-2,3,3a,4,6,6a- hexahydro-1H-furo[3,4-b]pyrrole-2- carbonyl]piperazine-1-carbonyl]-3- chlorophenyl]-5-amino-1-[2,3-difluoro-4- (fluoromethoxy)phenyl]imidazole-4- carboxamide |
| 200 | 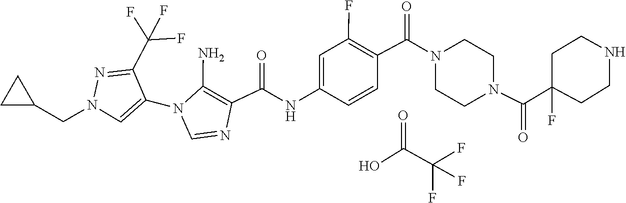 | 5-amino-1-[1-(cyclopropylmethyl)-3- (trifluoromethyl)pyrazol-4-yl]-N-[3-fluoro- 4-[4-(4-fluoropiperidine-4- carbonyl)piperazine-1- carbonyl]phenyl]imidazole-4- carboxamide; 2,2,2-trifluoroacetic acid |
| 201 | 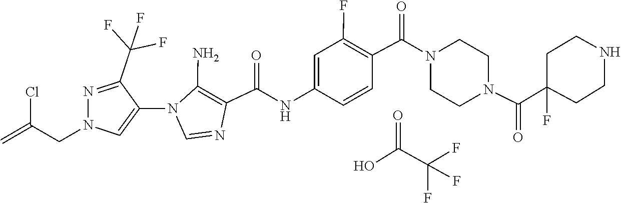 | 5-amino-1-[1-(2-chloroallyl)-3- (trifluoromethyl)pyrazol-4-yl]-N-[3-fluoro- 4-[4-(4-fluoropiperidine-4- carbonyl)piperazine-1- carbonyl]phenyl]imidazole-4- carboxamide; 1:1 2,2,2-trifluoroacetic acid |
| 202 | 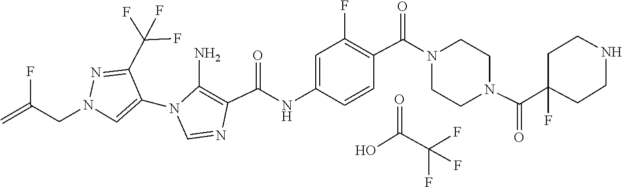 | 5-amino-1-[1-(2-fluoroallyl)-3- (trifluoromethyl)pyrazol-4-yl]-N-[3-fluoro- 4-[4-(4-fluoropiperidine-4- carbonyl)piperazine-1- carbonyl]phenyl]imidazole-4- carboxamide; 1:1 2,2,2-trifluoroacetic acid |
| 203 | 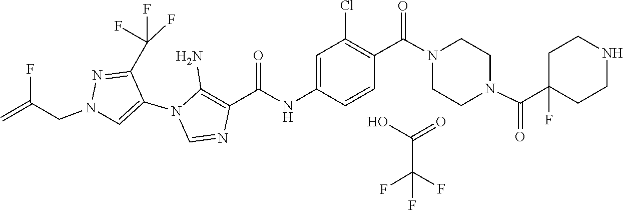 | 5-amino-N-[3-chloro-4-[4-(4- fluoropiperidine-4-carbonyl)piperazine- 1-carbonyl]phenyl]-1-[1-(2-fluoroallyl)-3- (trifluoromethyl)pyrazol-4-yl]imidazole-4- carboxamide; 1:1 2,2,2-trifluoroacetic acid |
| 204 | 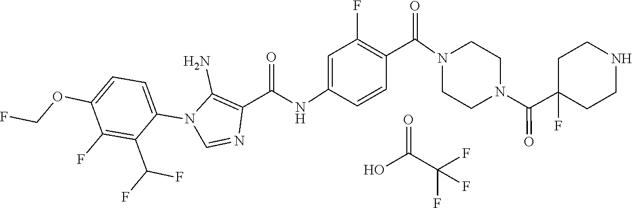 | 5-amino-1-[2-(difluoromethyl)-3-fluoro-4- (fluoromethoxy) phenyl]-N-[3-fluoro-4- [4-(4-fluoropiperidine-4- carbonyl)piperazine-1- carbonyl]phenyl]imidazole-4- carboxamide; 1:1 2,2,2-trifluoroacetic acid |
| 205 | 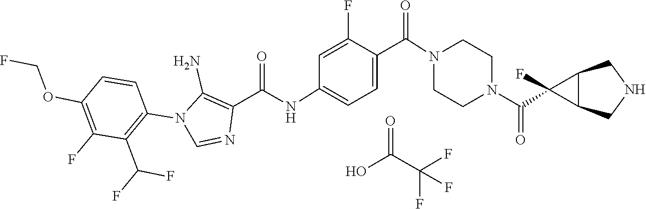 | 5-amino-1-[2-(difluoromethyl)-3-fluoro-4- (fluoromethoxy)phenyl]-N-[3-fluoro-4-[4- [(1R,5S,6r)-6-fluoro-3- azabicyclo[3.1.0]hexane-6- carbonyl]piperazine-1- carbonyl]phenyl]imidazole-4- carboxamide; 1:1 2,2,2-trifluoroacetic acid |
| 206 | 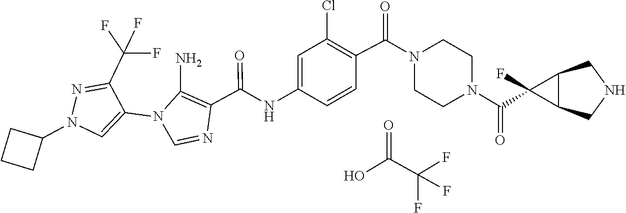 | 5-amino-N-[3-chloro-4-[4-[(1R,5S,6r)-6- fluoro-3-azabicyclo[3.1.0]hexane-6- carbonyl]piperazine-1-carbonyl]phenyl]- 1-[1-cyclobutyl-3- (trifluoromethyl)pyrazol-4-yl]imidazole-4- carboxamide; 1:1 2,2,2-trifluoroacetic acid |
| 207 | 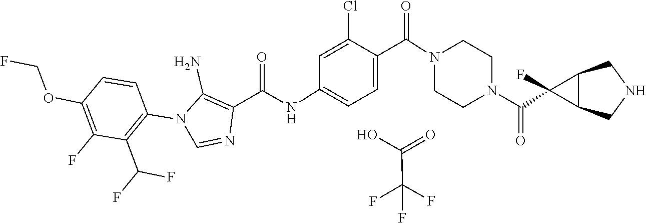 | 5-amino-N-[3-chloro-4-[4-[(1R,5S,6r)-6- fluoro-3-azabicyclo[3.1.0]hexane-6- carbonyl] piperazine-1-carbonyl]phenyl]- 1-[2-(difluoromethyl)-3-fluoro-4- (fluoromethoxy) phenyl]imidazole-4- carboxamide; 1:1 2,2,2-trifluoroacetic acid |
| 208 | 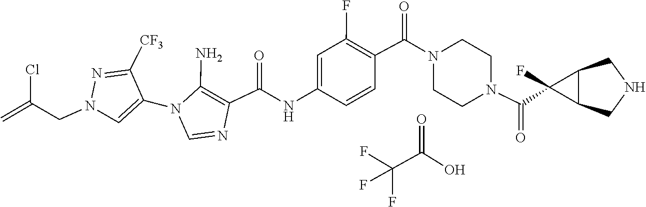 | 5-amino-1-(1-(2-chloroallyl)-3- (trifluoromethyl)-1H-pyrazol-4-yl)-N-(3- fluoro-4-(4-((1R,5S,6r)-6-fluoro-3- azabicyclo[3.1.0]hexane-6- carbonyl)piperazine-1-carbonyl)phenyl)- 1H-imidazole-4-carboxamide; 2,2,2- trifluoroacetic acid |
| 209 | 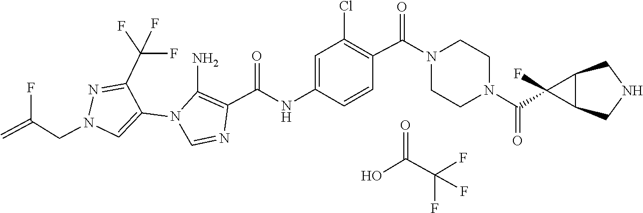 | 5-amino-N-[3-chloro-4-[4-[(1R,5S,6r)-6- fluoro-3-azabicyclo[3.1.0]hexane-6- carbonyl]piperazine-1-carbonyl]phenyl]- 1-[1-(2-fluoroallyl)-3- (trifluoromethyl)pyrazol-4-yl]imidazole-4- carboxamide; 1:1 2,2,2-trifluoroacetic acid |
| 210 | 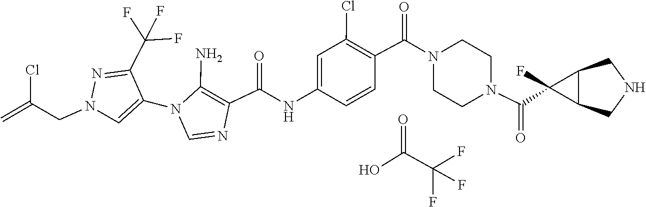 | 5-amino-1-[1-(2-chloroallyl)-3- (trifluoromethyl)pyrazol-4-yl]-N-[3- chloro-4-[4-[(1S,5R,6r)-6-fluoro-3- azabicyclo[3.1.0]hexane-6- carbonyl]piperazine-1- carbonyl]phenyl]imidazole-4- carboxamide; 2,2,2-trifluoroacetic acid |
| 211 | 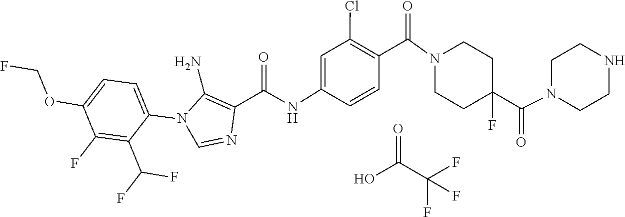 | 5-amino-N-[3-chloro-4-[4-fluoro-4- (piperazine-1-carbonyl)piperidine-1- carbonyl]phenyl]-1-[2-(difluoromethyl)-3- fluoro-4- (fluoromethoxy)phenyl]imidazole-4- carboxamide; 1:1 2,2,2-trifluoroacetic acid |
| 212 | 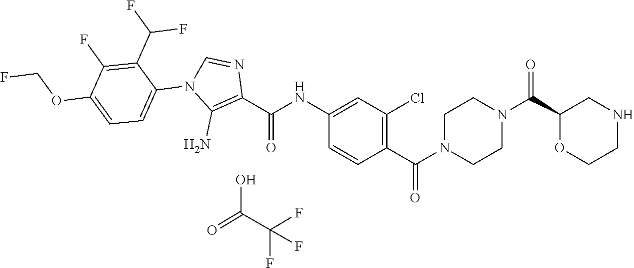 | (R)-5-amino-N-(3-chloro-4-(4- (morpholine-2-carbonyl)piperazine-1- carbonyl)phenyl)-1-(2-(difluoromethyl)- 3-fluoro-4-(fluoromethoxy)phenyl)-1H- imidazole-4-carboxamide; 2,2,2- trifluoroacetic acid |
| 213 | 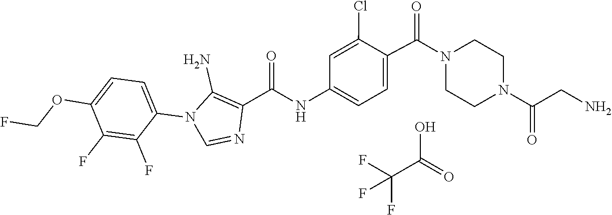 | 5-amino-N-(3-chloro-4-(4- glycylpiperazine-1-carbonyl)phenyl)-1- (2,3-difluoro-4-(fluoromethoxy)phenyl)- 1H-imidazole-4-carboxamide; 1:1 2,2,2- trifluoroacetate |
| 214 | 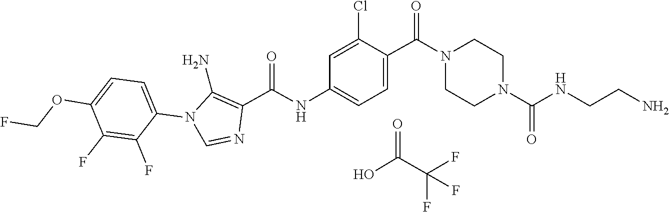 | 4-[4-[[5-amino-1-[2,3-difluoro-4- (fluoromethoxy)phenyl]imidazole-4- carbonyl]amino]-2-chlorobenzoyl]-N-(2- aminoethyl)piperazine-1-carboxamide; 1:1 2,2,2-trifluoroacetic acid |
| 215 | 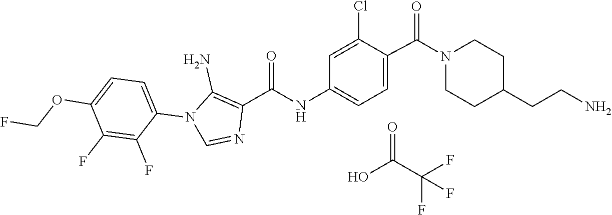 | 5-amino-N-[4-[4-(2- aminoethyl)piperidine-1-carbonyl]-3- chloro-phenyl]-1-[2,3-difluoro-4- (fluoromethoxy)phenyl]imidazole-4- carboxamide; 1:2 2,2,2-trifluoroacetic acid |
| 216 | 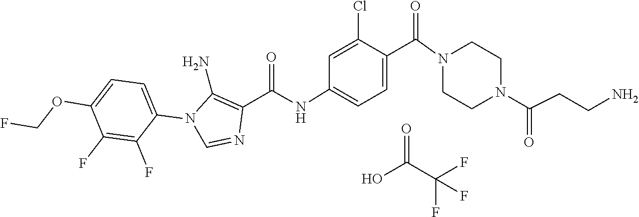 | 5-amino-N-[4-[4-(3-aminopropanoyl) piperazine-1-carbonyl]-3-chloro-phenyl]- 1-[2,3-difluoro-4- (fluoromethoxy)phenyl]imidazole-4- carboxamide; 1:1 2,2,2-trifluoroacetic acid |
| 217 | 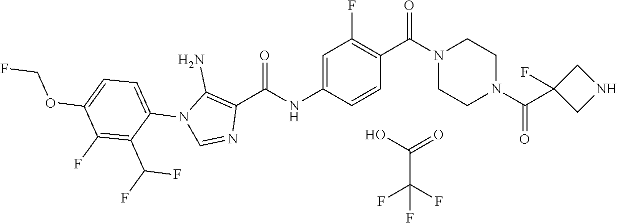 | 5-amino-1-[2-(difluoromethyl)-3-fluoro-4- (fluoromethoxy)phenyl]-N-[3-fluoro-4-[4- (3-fluoroazetidine-3- carbonyl)piperazine-1- carbonyl]phenyl]imidazole-4- carboxamide; 1:1 2,2,2-trifluoroacetic acid |
| 218 | 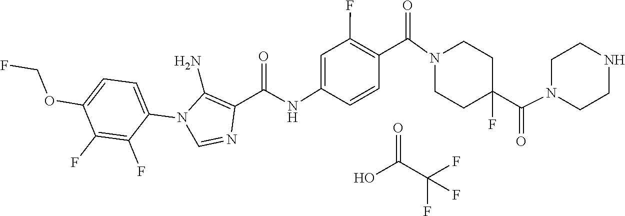 | 5-amino-1-[2,3-difluoro-4- (fluoromethoxy)phenyl]-N-[3-fluoro-4-[4- fluoro-4-(piperazine-1- carbonyl)piperidine-1- carbonyl]phenyl]imidazole-4- carboxamide; 1:1 2,2,2-trifluoroacetic acid |
| 219 | 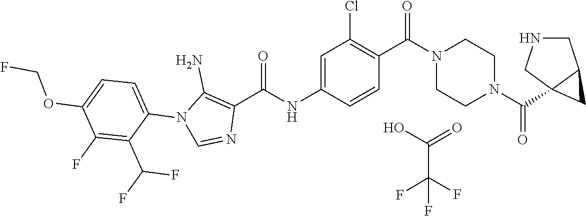 | 5-amino-N-[4-[4-[(1S,5S)-3- azabicyclo[3.1.0]hexane-1- carbonyl]piperazine-1-carbonyl]-3- chloro-phenyl]-1-[2-(difluoromethyl)-3- fluoro-4- (fluoromethoxy)phenyl]imidazole-4- carboxamide; 1:1 2,2,2-trifluoroacetic acid |
| 220 | 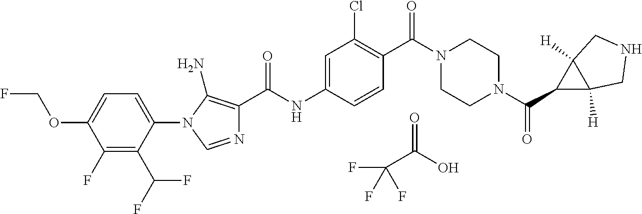 | N-(4-(4-((1R,5S,6s)-3- azabicyclo[3.1.0]hexane-6- carbonyl)piperazine-1-carbonyl)-3- chlorophenyl)-5-amino-1-(2- (difluoromethyl)-3-fluoro-4- (fluoromethoxy)phenyl)-1H-imidazole-4- carboxamide; 2,2,2-trifluoroacetic acid |
| 221 | 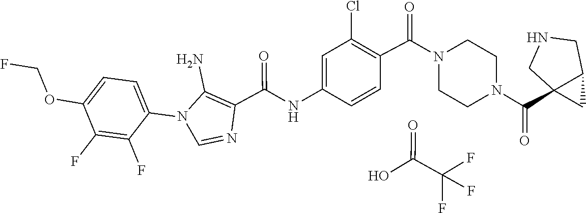 | 5-amino-N-[4-[4-[(1R,5R)-3- azabicyclo[3.1.0]hexane-1- carbonyl]piperazine-1-carbonyl]-3- chloro-phenyl]-1-[2,3-difluoro-4- (fluoromethoxy)phenyl]imidazole-4- carboxamide; 1:1 2,2,2-trifluoroacetic acid |
| 222 | 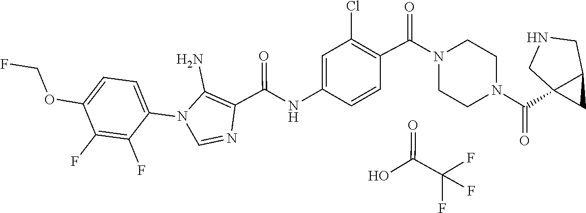 | 5-amino-N-[4-[4-[(1S,5S)-3- azabicyclo[3.1.0]hexane-1- carbonyl]piperazine-1-carbonyl]-3- chloro-phenyl]-1-[2,3-difluoro-4- (fluoromethoxy)phenyl]imidazole-4- carboxamide; 1:1 2,2,2-trifluoroacetic acid |
| 223 | 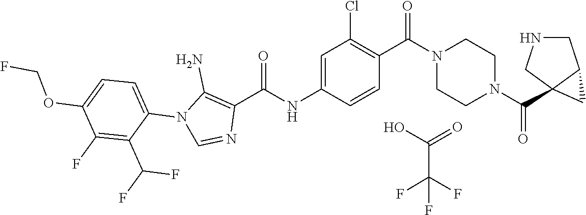 | 5-amino-N-[4-[4-[(1R,5R)-3- azabicyclo[3.1.0]hexane-1- carbonyl]piperazine-1-carbonyl]-3- chloro-phenyl]-1-[2-(difluoromethyl)-3- fluoro-4- (fluoromethoxy)phenyl]imidazole-4- carboxamide; 1:1 2,2,2-trifluoroacetic acid |
| 224 | 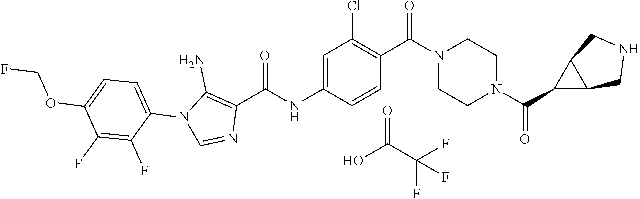 | 5-amino-N-[4-[4-[(1R,5S,6r)-3- azabicyclo[3.1.0]hexane-6- carbonyl]piperazine-1-carbonyl]-3- chloro-phenyl]-1-[2,3-difluoro-4- (fluoromethoxy)phenyl]imidazole-4- carboxamide; 1:1 2,2,2-trifluoroacetic acid |
| 225 | 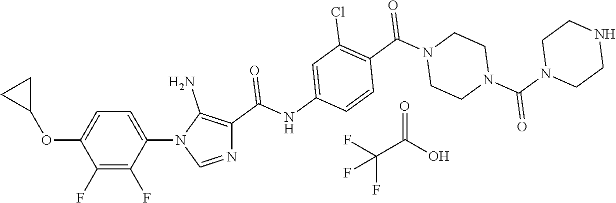 | 5-amino-N-[3-chloro-4-[4-(piperazine-1- carbonyl) piperazine-1-carbonyl] phenyl]-1-[4-(cyclopropoxy)-2,3-difluoro- phenyl] imidazole-4-carboxamide; 1:1 2,2,2-trifluoroacetic acid |
| 226 | 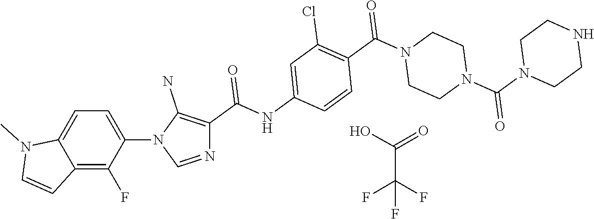 | 5-amino-N-[3-chloro-4-[4-(piperazine-1- carbonyl)piperazine-1-carbonyl]phenyl]- 1-(4-fluoro-1-methyl-indol-5- yl)imidazole-4-carboxamide; 1:1 2,2,2- trifluoroacetic acid |
| 228 | 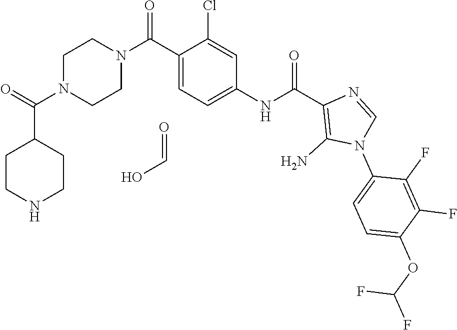 | 5-amino-N-[3-chloro-4-[4-(piperidine-4- carbonyl)piperazine-1-carbonyl]phenyl]- 1-[4-(difluoromethoxy)-2,3- difluorophenyl]imidazole-4- carboxamide; formic acid |
| 229 | 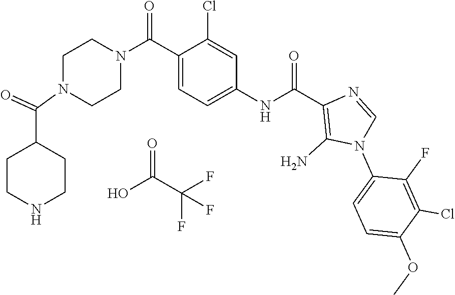 [SMILES: COc1ccc(-n2cnc(C(=O)Nc3ccc(C(=O)N4CCN(C(=O)C5CCNCC5)CC4)c(Cl)c3)c2N)c(F)c1Cl ; O=C(O)C(F)(F)F] | 5-amino-1-(3-chloro-2-fluoro-4- methoxyphenyl)-N-[3-chloro-4-[4- (piperidine-4-carbonyl)piperazine-1- carbonyl]phenyl]imidazole-4- carboxamide; 2,2,2-trifluoroacetic acid |
| 230 | 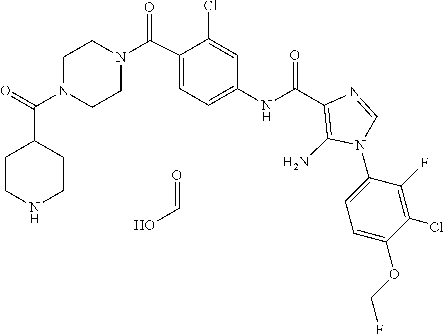 [SMILES: Nc1c(C(=O)Nc2ccc(C(=O)N3CCN(C(=O)C4CCNCC4)CC3)c(Cl)c2)ncn1-c1ccc(OCF)c(Cl)c1F ; O=CO] | 5-amino-1-[3-chloro-2-fluoro-4- (fluoromethoxy)phenyl]-N-[3-chloro-4-[4- (piperidine-4-carbonyl)piperazine-1- carbonyl]phenyl]imidazole-4- carboxamide; formic acid |
| 231 | 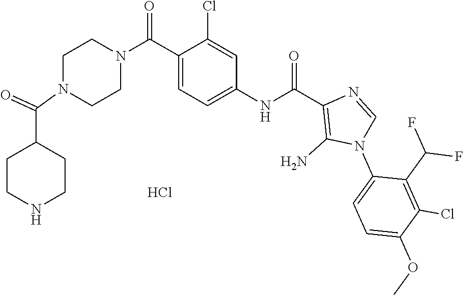 [SMILES: COc1ccc(-n2cnc(C(=O)Nc3ccc(C(=O)N4CCN(C(=O)C5CCNCC5)CC4)c(Cl)c3)c2N)c(C(F)F)c1Cl ; Cl] | 5-amino-1-[3-chloro-2-(difluoromethyl)- 4-methoxyphenyl]-N-[3-chloro-4-[4- (piperidine-4-carbonyl)piperazine-1- carbonyl]phenyl]imidazole-4- carboxamide; hydrochloride |
| 232 | 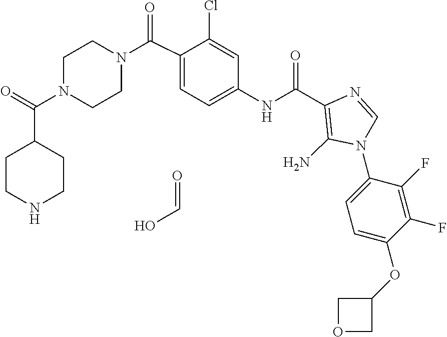 [SMILES: Nc1c(C(=O)Nc2ccc(C(=O)N3CCN(C(=O)C4CCNCC4)CC3)c(Cl)c2)ncn1-c1ccc(OC2COC2)c(F)c1F ; O=CO] | 5-amino-N-[3-chloro-4-[4-(piperidine-4- carbonyl)piperazine-1-carbonyl]phenyl]- 1-[2,3-difluoro-4-(oxetan-3- yloxy)phenyl]imidazole-4- carboxamide; formic acid |
| 233 | 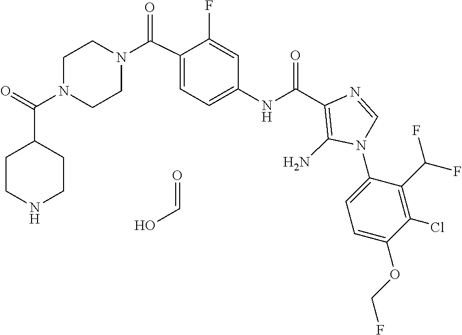 [SMILES: Nc1c(C(=O)Nc2ccc(C(=O)N3CCN(C(=O)C4CCNCC4)CC3)c(F)c2)ncn1-c1ccc(OCF)c(Cl)c1C(F)F ; O=CO] | 5-amino-1-[3-chloro-2-(difluoromethyl)- 4-(fluoromethoxy)phenyl]-N-[3-fluoro-4- [4-(piperidine-4-carbonyl)piperazine-1- carbonyl]phenyl]imidazole-4- carboxamide; formic acid |
| 234 | 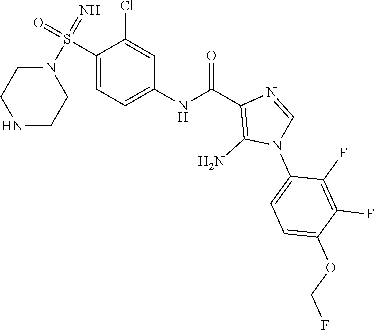 [SMILES: N=S(=O)(c1ccc(NC(=O)c2ncn(-c3ccc(OCF)c(F)c3F)c2N)cc1Cl)N1CCNCC1] | 5-amino-N-[3-chloro-4-(piperazin-1- ylsulfonimidoyl)phenyl]-1-[2,3-difluoro-4- (fluoromethoxy)phenyl]imidazole-4- carboxamide |
| 235 | 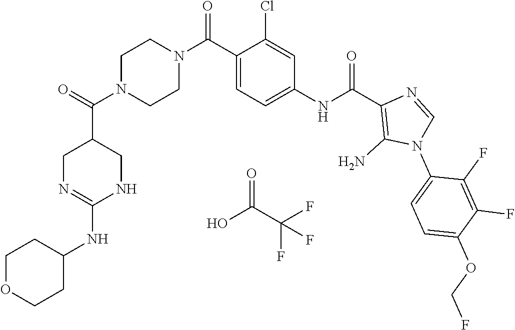 [SMILES: Nc1c(C(=O)Nc2ccc(C(=O)N3CCN(C(=O)C4CN=C(NC5CCOCC5)NC4)CC3)c(Cl)c2)ncn1-c1ccc(OCF)c(F)c1F ; O=C(O)C(F)(F)F] | 5-amino-N-[3-chloro-4-[4-[2- (tetrahydropyran-4-ylamino)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 237 | 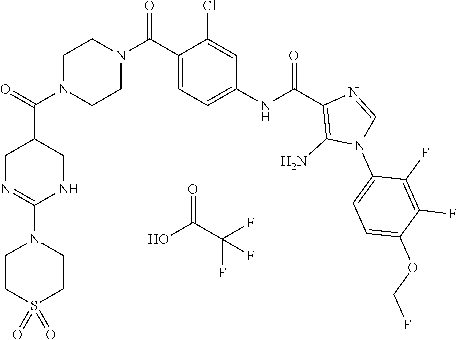 [SMILES: Nc1c(C(=O)Nc2ccc(C(=O)N3CCN(C(=O)C4CN=C(N5CCS(=O)(=O)CC5)NC4)CC3)c(Cl)c2)ncn1-c1ccc(OCF)c(F)c1F ; O=C(O)C(F)(F)F] | 5-amino-N-[3-chloro-4-[4-[2-(1-imino-1- oxo-1,4-thiazinan-4-yl)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 238 | 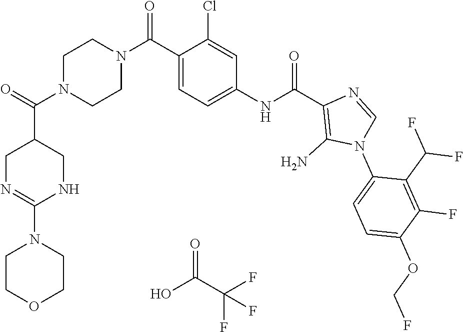 [SMILES: Nc1c(C(=O)Nc2ccc(C(=O)N3CCN(C(=O)C4CN=C(N5CCOCC5)NC4)CC3)c(Cl)c2)ncn1-c1ccc(OCF)c(F)c1C(F)F ; O=C(O)C(F)(F)F] | 5-amino-N-[3-chloro-4-[4-(2-morpholino- 1,4,5,6-tetrahydropyrimidine-5- carbonyl)piperazine-1-carbonyl]phenyl]- 1-[2-(difluoromethyl)-3-fluoro-4- (fluoromethoxy)phenyl]imidazole-4- carboxamide; 2,2,2-trifluoroacetic acid |
| 239 | 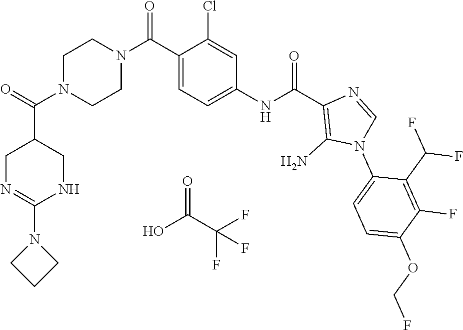 [SMILES: Nc1c(C(=O)Nc2ccc(C(=O)N3CCN(C(=O)C4CN=C(N5CCC5)NC4)CC3)c(Cl)c2)ncn1-c1ccc(OCF)c(F)c1C(F)F ; O=C(O)C(F)(F)F] | 5-amino-N-[3-chloro-4-[4-(2-morpholino- 1,4,5,6-tetrahydropyrimidine-5- carbonyl)piperazine-1-carbonyl]phenyl]- 1-[2-(difluoromethyl)-3-fluoro-4- (fluoromethoxy)phenyl]imidazole-4- carboxamide; 2,2,2-trifluoroacetic acid |
| 240 | 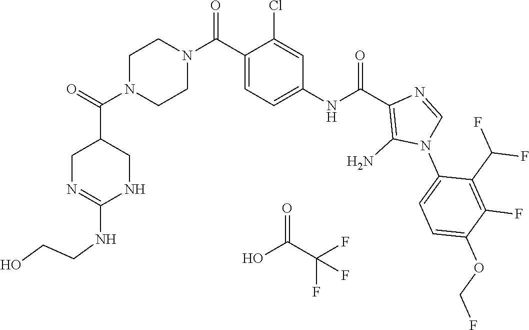 [SMILES: Nc1c(C(=O)Nc2ccc(C(=O)N3CCN(C(=O)C4CN=C(NCCO)NC4)CC3)c(Cl)c2)ncn1-c1ccc(OCF)c(F)c1C(F)F ; O=C(O)C(F)(F)F] | 5-amino-N-[4-[4-[2-(azetidin-1-yl)- 1,4,5,6-tetrahydropyrimidine-5- carbonyl]piperazine-1-carbonyl]-3- chlorophenyl]-1-[2-(difluoromethyl)-3- fluoro-4- (fluoromethoxy)phenyl]imidazole-4- carboxamide; 2,2,2-trifluoroacetic acid |
| 241 | 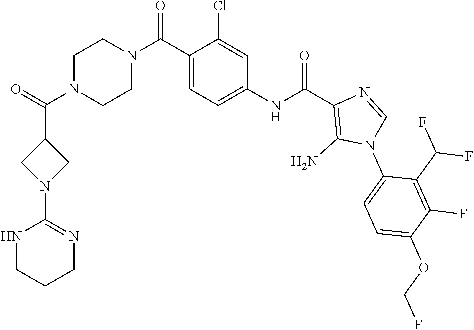 [SMILES: Nc1c(C(=O)Nc2ccc(C(=O)N3CCN(C(=O)C4CN(C5=NCCCN5)C4)CC3)c(Cl)c2)ncn1-c1ccc(OCF)c(F)c1C(F)F] | 5-amino-N-[3-chloro-4-[4-[2-(2- hydroxyethylamino)-1,4,5,6- tetrahydropyrimidine-5- carbonyl]piperazine-1-carbonyl]phenyl]- 1-[2-(difluoromethyl)-3-fluoro-4- (fluoromethoxy)phenyl]imidazole-4- carboxamide; 2,2,2-trifluoroacetic acid |
| 242 | 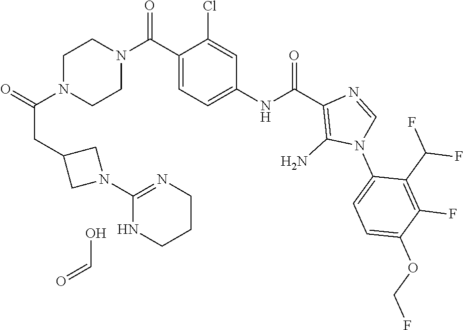 | 5-amino-N-[3-chloro-4-[4-[1-(1,4,5,6- tetrahydropyrimidin-2-yl)azetidine-3- carbonyl]piperazine-1-carbonyl]phenyl]- 1-[2-(difluoromethyl)-3-fluoro-4- (fluoromethoxy)phenyl]imidazole-4- carboxamide |
| 243 | 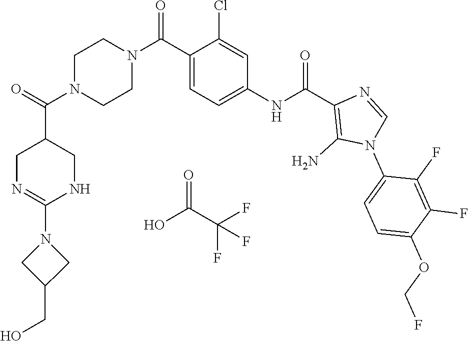 [SMILES: Nc1c(C(=O)Nc2ccc(C(=O)N3CCN(C(=O)C4CN=C(N5CC(CO)C5)NC4)CC3)c(Cl)c2)ncn1-c1ccc(OCF)c(F)c1F ; O=C(O)C(F)(F)F] | 5-amino-N-[3-chloro-4-[4-[2-[1-(1,4,5,6- tetrahydropyrimidin-2-yl)azetidin-3- yl]acetyl]piperazine-1-carbonyl]phenyl]- 1-[2-(difluoromethyl)-3-fluoro-4- (fluoromethoxy)phenyl]imidazole-4- carboxamide; formic acid |
| 244 | 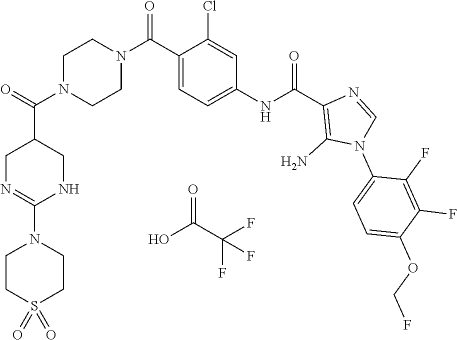 [SMILES: Nc1c(C(=O)Nc2ccc(C(=O)N3CCN(C(=O)C4CN=C(N5CCS(=O)(=O)CC5)NC4)CC3)c(Cl)c2)ncn1-c1ccc(OCF)c(F)c1F ; O=C(O)C(F)(F)F] | 5-amino-N-[3-chloro-4-[4-[2-[3- (hydroxymethyl)azetidin-1-yl]-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 245 | 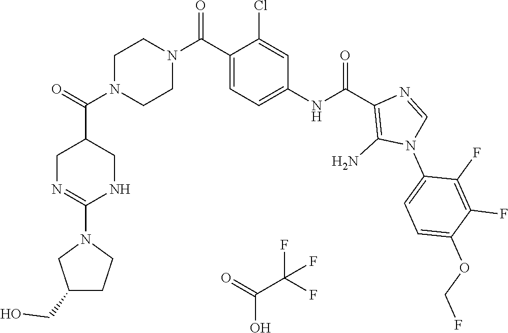 [SMILES: Nc1c(C(=O)Nc2ccc(C(=O)N3CCN(C(=O)C4CN=C(N5CC[C@H](CO)C5)NC4)CC3)c(Cl)c2)ncn1-c1ccc(OCF)c(F)c1F ; O=C(O)C(F)(F)F] | 5-amino-N-[3-chloro-4-[4-[2-(1,1-dioxo- 1,4-thiazinan-4-yl)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 246 | 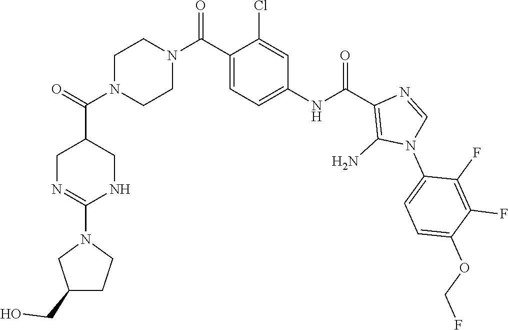 [SMILES: Nc1c(C(=O)Nc2ccc(C(=O)N3CCN(C(=O)C4CN=C(N5CC[C@@H](CO)C5)NC4)CC3)c(Cl)c2)ncn1-c1ccc(OCF)c(F)c1F] | 5-amino-N-[3-chloro-4-[4-[2-[(3S)-3- (hydroxymethyl)pyrrolidin-1-yl]-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 247 | 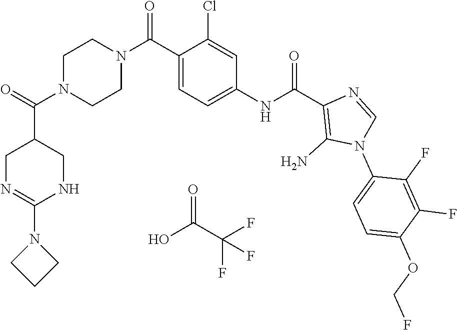 [SMILES: Nc1c(C(=O)Nc2ccc(C(=O)N3CCN(C(=O)C4CN=C(N5CCC5)NC4)CC3)c(Cl)c2)ncn1-c1ccc(OCF)c(F)c1F ; O=C(O)C(F)(F)F] | 5-amino-N-[3-chloro-4-[4-[2-[(3R)-3- (hydroxymethyl)pyrrolidin-1-yl]-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide |
| 248 | 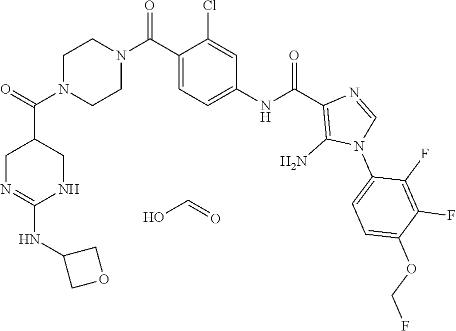 [SMILES: Nc1c(C(=O)Nc2ccc(C(=O)N3CCN(C(=O)C4CN=C(NC5COC5)NC4)CC3)c(Cl)c2)ncn1-c1ccc(OCF)c(F)c1F ; O=CO] | 5-amino-N-[4-[4-[2-(azetidin-1-yl)- 1,4,5,6-tetrahydropyrimidine-5- carbonyl]piperazine-1-carbonyl]-3- chlorophenyl]-1-[2,3-difluoro-4- (fluoromethoxy)phenyl]imidazole-4- carboxamide; 2,2,2-trifluoroacetic acid |
| 249 | 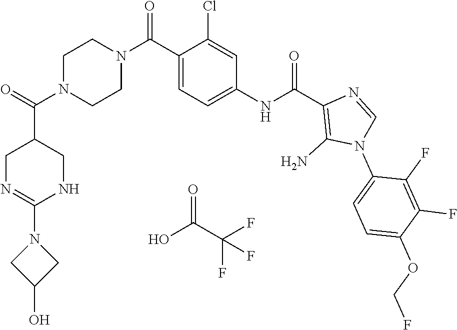 [SMILES: Nc1c(C(=O)Nc2ccc(C(=O)N3CCN(C(=O)C4CN=C(N5CC(O)C5)NC4)CC3)c(Cl)c2)ncn1-c1ccc(OCF)c(F)c1F ; O=C(O)C(F)(F)F] | 5-amino-N-[3-chloro-4-[4-[2-(oxetan-3- ylamino)-1,4,5,6-tetrahydropyrimidine-5- carbonyl]piperazine-1-carbonyl]phenyl]- 1-[2,3-difluoro-4- (fluoromethoxy)phenyl]imidazole-4- carboxamide; formic acid |
| 250 | 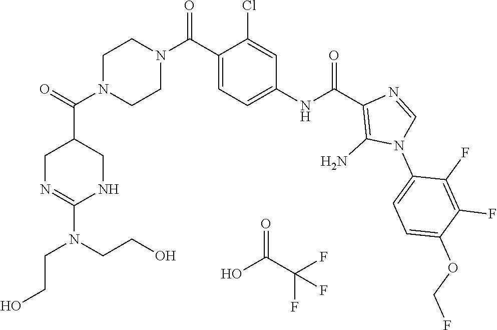 [SMILES: Nc1c(C(=O)Nc2ccc(C(=O)N3CCN(C(=O)C4CN=C(N(CCO)CCO)NC4)CC3)c(Cl)c2)ncn1-c1ccc(OCF)c(F)c1F ; O=C(O)C(F)(F)F] | 5-amino-N-[3-chloro-4-[4-[2-(3- hydroxyazetidin-1-yl)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 251 | 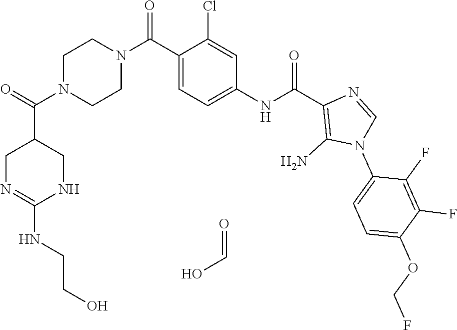 [SMILES: Nc1c(C(=O)Nc2ccc(C(=O)N3CCN(C(=O)C4CN=C(NCCO)NC4)CC3)c(Cl)c2)ncn1-c1ccc(OCF)c(F)c1F ; O=CO] | 5-amino-N-[4-[4-[2-[bis(2- hydroxyethyl)amino]-1,4,5,6- tetrahydropyrimidine-5- carbonyl]piperazine-1-carbonyl]-3- chlorophenyl]-1-[2,3-difluoro-4- (fluoromethoxy)phenyl]imidazole-4- carboxamide; 2,2,2-trifluoroacetic acid |
| 252 | 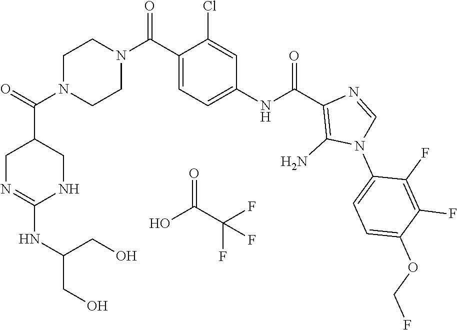 [SMILES: Nc1c(C(=O)Nc2ccc(C(=O)N3CCN(C(=O)C4CN=C(NC(CO)CO)NC4)CC3)c(Cl)c2)ncn1-c1ccc(OCF)c(F)c1F ; O=C(O)C(F)(F)F] | 5-amino-N-[3-chloro-4-[4-[2-(2- hydroxyethylamino)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; formic acid |
| 253 | 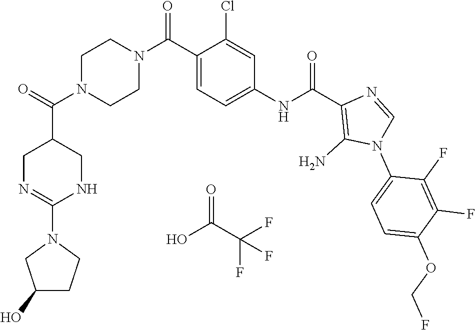 [SMILES: Nc1c(C(=O)Nc2ccc(C(=O)N3CCN(C(=O)C4CN=C(N5CC[C@@H](O)C5)NC4)CC3)c(Cl)c2)ncn1-c1ccc(OCF)c(F)c1F ; O=C(O)C(F)(F)F] | 5-amino-N-[3-chloro-4-[4-[2-[[2-hydroxy- 1-(hydroxymethyl)ethyl]amino]-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 254 | 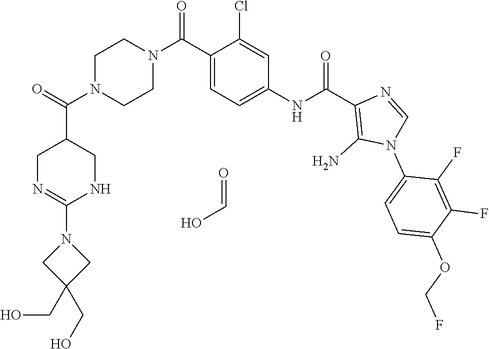 [SMILES: Nc1c(C(=O)Nc2ccc(C(=O)N3CCN(C(=O)C4CN=C(N5CC(CO)(CO)C5)NC4)CC3)c(Cl)c2)ncn1-c1ccc(OCF)c(F)c1F ; O=CO] | 5-amino-N-[3-chloro-4-[4-[2-[(3R)-3- hydroxypyrrolidin-1-yl]-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 255 | 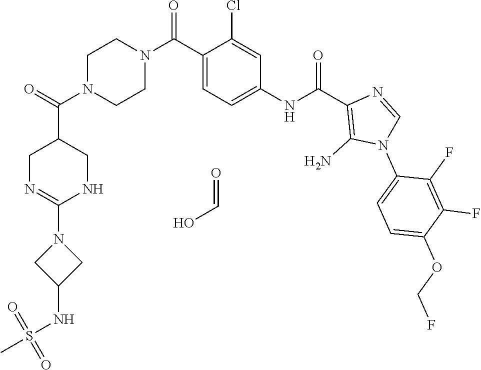 [SMILES: CS(=O)(=O)NC1CN(C2=NCC(C(=O)N3CCN(C(=O)c4ccc(NC(=O)c5ncn(-c6ccc(OCF)c(F)c6F)c5N)cc4Cl)CC3)CN2)C1 ; O=CO] | 5-amino-N-[4-[4-[2-[3,3- bis(hydroxymethyl)azetidin-1-yl]-1,4,5,6- tetrahydropyrimidine-5- carbonyl]piperazine-1-carbonyl]-3- chlorophenyl]-1-[2,3-difluoro-4- (fluoromethoxy)phenyl]imidazole-4- carboxamide; formic acid |
| 256 | 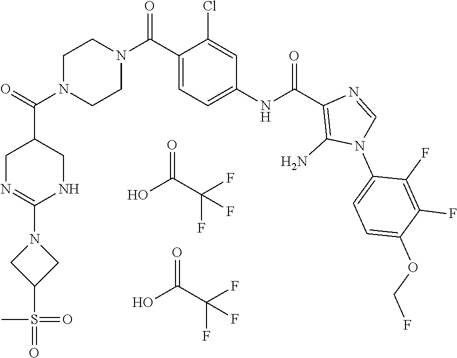 | 5-amino-N-[3-chloro-4-[4-[2-[3- (methanesulfonamido)azetidin-1-yl]- 1,4,5,6-tetrahydropyrimidine-5- carbonyl]piperazine-1-carbonyl]phenyl]- 1-[2,3-difluoro-4- (fluoromethoxy)phenyl]imidazole-4- carboxamide; formic acid |
| 257 | 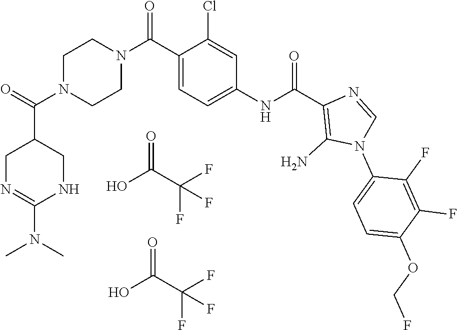 | 5-amino-N-[3-chloro-4-[4-[2-(3- methylsulphonylazetidin-1-yl)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; bis(2,2,2-trifluoroacetic acid) |
| 258 | 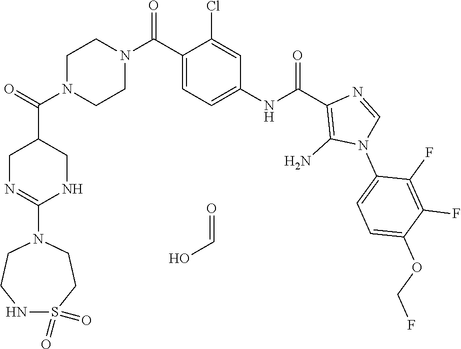 [SMILES: Nc1c(C(=O)Nc2ccc(C(=O)N3CCN(C(=O)C4CN=C(N5CCNS(=O)(=O)CC5)NC4)CC3)c(Cl)c2)ncn1-c1ccc(OCF)c(F)c1F ; O=CO] | 5-amino-N-[3-chloro-4-[4-[2- (dimethylamino)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; bis(2,2,2-trifluoroacetic acid) |
| 259 | 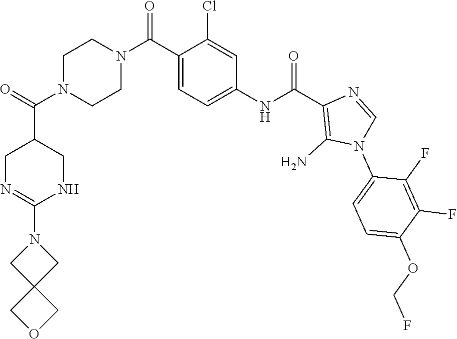 [SMILES: Nc1c(C(=O)Nc2ccc(C(=O)N3CCN(C(=O)C4CN=C(N5CC6(COC6)C5)NC4)CC3)c(Cl)c2)ncn1-c1ccc(OCF)c(F)c1F] | 5-amino-N-[3-chloro-4-[4-[2-(1,1-dioxo- 1,2,5-thiadiazepan-5-yl)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; formic acid |
| 260 | 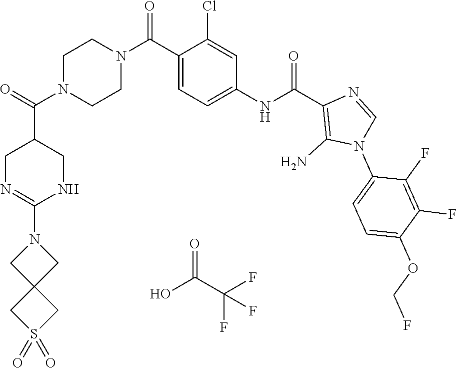 [SMILES: Nc1c(C(=O)Nc2ccc(C(=O)N3CCN(C(=O)C4CN=C(N5CC6(C5)CS(=O)(=O)C6)NC4)CC3)c(Cl)c2)ncn1-c1ccc(OCF)c(F)c1F ; O=C(O)C(F)(F)F] | 5-amino-N-[3-chloro-4-[4-[2-(2-oxa-6- azaspiro[3.3]heptan-6-yl)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide |
| 261 | 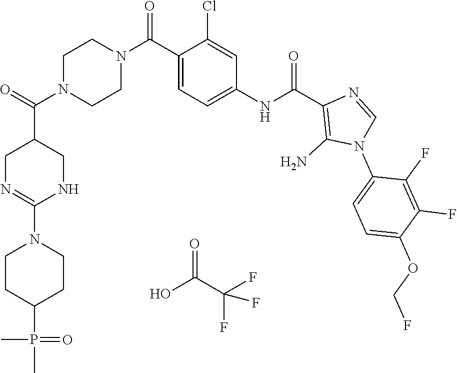 [SMILES: CP(C)(=O)C1CCN(C2=NCC(C(=O)N3CCN(C(=O)c4ccc(NC(=O)c5ncn(-c6ccc(OCF)c(F)c6F)c5N)cc4Cl)CC3)CN2)CC1 ; O=C(O)C(F)(F)F] | 5-amino-N-[3-chloro-4-[4-[2-(2,2-dioxo- 2λ6-thia-6-azaspiro[3.3]heptan-6-yl)- 1,4,5,6-tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 262 | 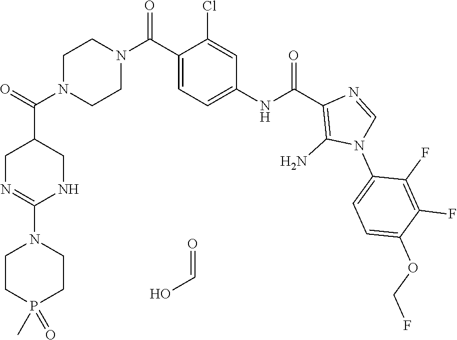 [SMILES: CP1(=O)CCN(C2=NCC(C(=O)N3CCN(C(=O)c4ccc(NC(=O)c5ncn(-c6ccc(OCF)c(F)c6F)c5N)cc4Cl)CC3)CN2)CC1 ; O=CO] | 5-amino-N-[3-chloro-4-[4-[2-(4- dimethylphosphoryl-1-piperidyl)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 263 | 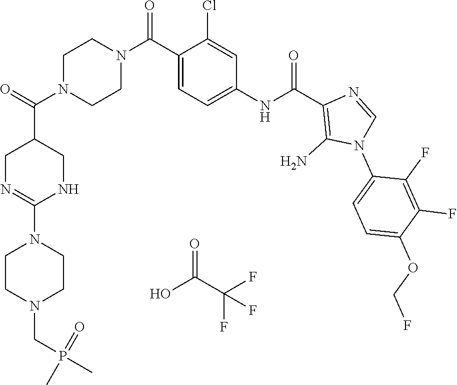 [SMILES: CP(C)(=O)CN1CCN(C2=NCC(C(=O)N3CCN(C(=O)c4ccc(NC(=O)c5ncn(-c6ccc(OCF)c(F)c6F)c5N)cc4Cl)CC3)CN2)CC1 ; O=C(O)C(F)(F)F] | 5-amino-N-[3-chloro-4-[4-[2-[4- (dimethylphosphorylmethyl)piperazin-1- yl]-1,4,5,6-tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 264 | 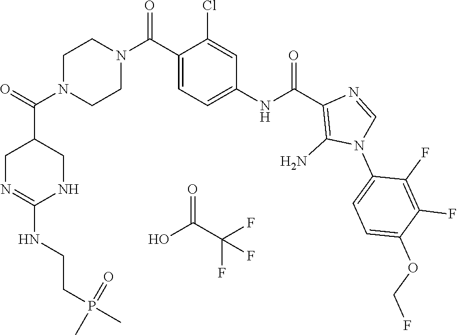 [SMILES: CP(C)(=O)CCNC1=NCC(C(=O)N2CCN(C(=O)c3ccc(NC(=O)c4ncn(-c5ccc(OCF)c(F)c5F)c4N)cc3Cl)CC2)CN1 ; O=C(O)C(F)(F)F] | 5-amino-N-[3-chloro-4-[4-[2-(2- dimethylphosphorylethylamino)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 265 | 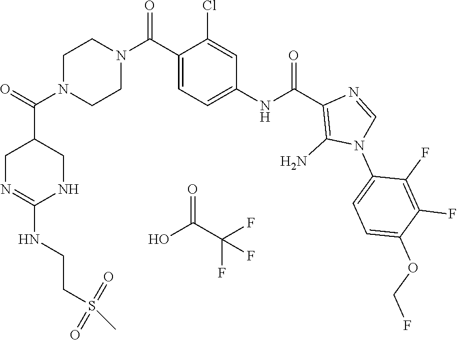 [SMILES: CS(=O)(=O)CCNC1=NCC(C(=O)N2CCN(C(=O)c3ccc(NC(=O)c4ncn(-c5ccc(OCF)c(F)c5F)c4N)cc3Cl)CC2)CN1 ; O=C(O)C(F)(F)F] | 5-amino-N-[3-chloro-4-[4-[2-(2- methylsulphonylethylamino)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 266 | 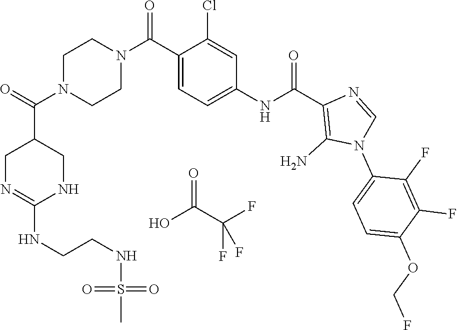 [SMILES: CS(=O)(=O)NCCNC1=NCC(C(=O)N2CCN(C(=O)c3ccc(NC(=O)c4ncn(-c5ccc(OCF)c(F)c5F)c4N)cc3Cl)CC2)CN1 ; O=C(O)C(F)(F)F] | 5-amino-N-[3-chloro-4-[4-[2-[2- (methanesulfonamido)ethylamino]- 1,4,5,6-tetrahydropyrimidine-5- carbonyl]piperazine-1-carbonyl]phenyl]- 1-[2,3-difluoro-4- (fluoromethoxy)phenyl]imidazole-4- carboxamide; 2,2,2-trifluoroacetic acid |
| 267 | 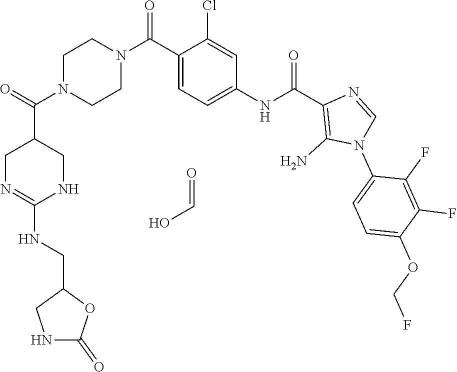 [SMILES: Nc1c(C(=O)Nc2ccc(C(=O)N3CCN(C(=O)C4CN=C(NCC5CNC(=O)O5)NC4)CC3)c(Cl)c2)ncn1-c1ccc(OCF)c(F)c1F ; O=CO] | 5-amino-N-[3-chloro-4-[4-[2-[(2- oxooxazolidin-5-yl)methylamino]- 1,4,5,6-tetrahydropyrimidine-5- carbonyl]piperazine-1-carbonyl]phenyl]- 1-[2,3-difluoro-4- (fluoromethoxy)phenyl]imidazole-4- carboxamide; formic acid |
| 268 | 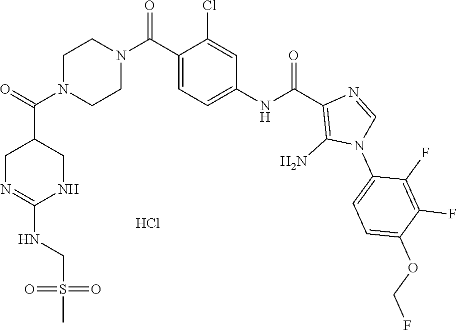 [SMILES: CS(=O)(=O)CNC1=NCC(C(=O)N2CCN(C(=O)c3ccc(NC(=O)c4ncn(-c5ccc(OCF)c(F)c5F)c4N)cc3Cl)CC2)CN1 ; Cl] | 5-amino-N-[3-chloro-4-[4-[2- (methylsulphonylmethylamino)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; hydrochloride |
| 269 | 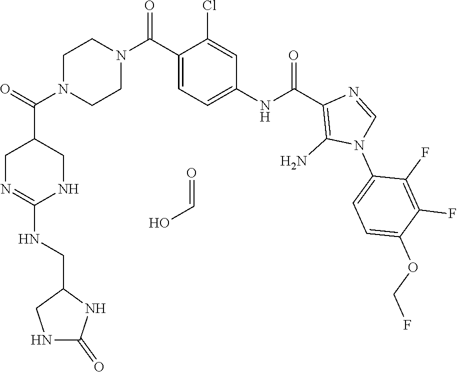 [SMILES: Nc1c(C(=O)Nc2ccc(C(=O)N3CCN(C(=O)C4CN=C(NCC5CNC(=O)N5)NC4)CC3)c(Cl)c2)ncn1-c1ccc(OCF)c(F)c1F ; O=CO] | 5-amino-N-[3-chloro-4-[4-[2-[(2- oxooxazolidin-4-yl)methylamino]- 1,4,5,6-tetrahydropyrimidine-5- carbonyl]piperazine-1-carbonyl]phenyl]- 1-[2,3-difluoro-4- (fluoromethoxy)phenyl]imidazole-4- carboxamide; formic acid |
| 270 | 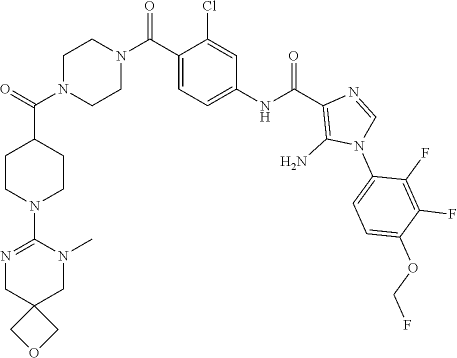 [SMILES: CN1CC2(CN=C1N1CCC(C(=O)N3CCN(C(=O)c4ccc(NC(=O)c5ncn(-c6ccc(OCF)c(F)c6F)c5N)cc4Cl)CC3)CC1)COC2] | 5-amino-N-[3-chloro-4-[4-[1-(6-methyl-2- oxa-6,8-diazaspiro[3.5]non-7-en-7- yl)piperidine-4-carbonyl]piperazine-1- carbonyl]phenyl]-1-[2,3-difluoro-4- (fluoromethoxy)phenyl]imidazole-4- carboxamide |
| 271 | 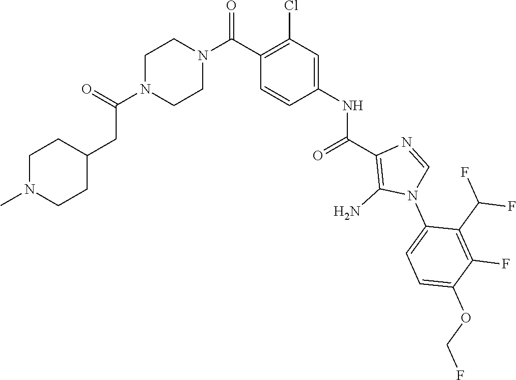 [SMILES: CN1CCC(CC(=O)N2CCN(C(=O)c3ccc(NC(=O)c4ncn(-c5ccc(OCF)c(F)c5C(F)F)c4N)cc3Cl)CC2)CC1] | 5-amino-N-[3-chloro-4-[4-[2-(1-methyl-4- piperidyl)acetyl]piperazine-1- carbonyl]phenyl]-1-[2-(difluoromethyl)-3- fluoro-4- (fluoromethoxy)phenyl]imidazole-4- carboxamide |
| 272 | 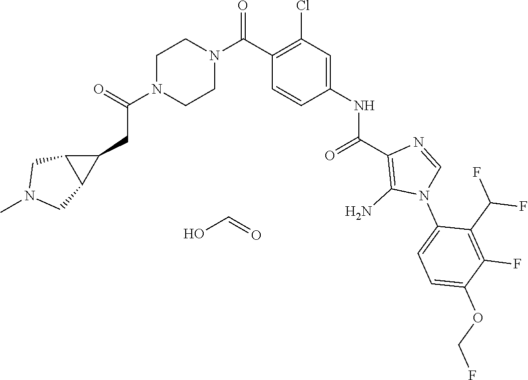 [SMILES: CN1C[C@@H]2[C@@H](CC(=O)N3CCN(C(=O)c4ccc(NC(=O)c5ncn(-c6ccc(OCF)c(F)c6C(F)F)c5N)cc4Cl)CC3)[C@@H]2C1 ; O=CO] | 5-amino-N-[3-chloro-4-[4-[2-[(1R,5S)-3- methyl-3-azabicyclo[3.1.0]hexan-6- yl]acetyl]piperazine-1-carbonyl]phenyl]- 1-[2-(difluoromethyl)-3-fluoro-4- (fluoromethoxy)phenyl]imidazole-4- carboxamide; formic acid |
| 273 | 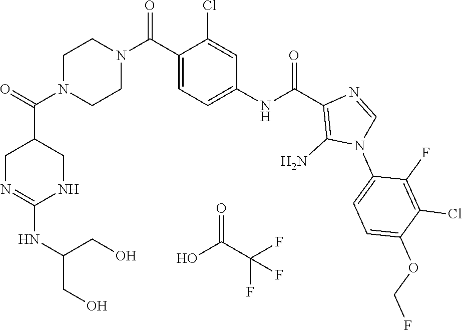 [SMILES: Nc1c(C(=O)Nc2ccc(C(=O)N3CCN(C(=O)C4CN=C(NC(CO)CO)NC4)CC3)c(Cl)c2)ncn1-c1ccc(OCF)c(Cl)c1F ; O=C(O)C(F)(F)F] | 5-amino-1-[3-chloro-2-fluoro-4- (fluoromethoxy)phenyl]-N-[3-chloro-4-[4- [2-[[2-hydroxy-1- (hydroxymethyl)ethyl]amino]-1,4,5,6- tetrahydropyrimidine-5- carbonyl]piperazine-1- carbonyl]phenyl]imidazole-4- carboxamide; 2,2,2-trifluoroacetic acid |
| 274 | 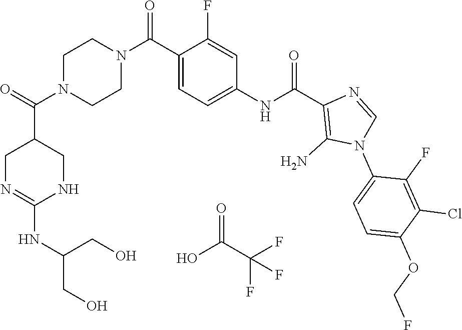 [SMILES: Nc1c(C(=O)Nc2ccc(C(=O)N3CCN(C(=O)C4CN=C(NC(CO)CO)NC4)CC3)c(F)c2)ncn1-c1ccc(OCF)c(Cl)c1F ; O=C(O)C(F)(F)F] | 5-amino-1-[3-chloro-2-fluoro-4- (fluoromethoxy)phenyl]-N-[3-fluoro-4-[4- [2-[[2-hydroxy-1- (hydroxymethyl)ethyl]amino]-1,4,5,6- tetrahydropyrimidine-5- carbonyl]piperazine-1- carbonyl]phenyl]imidazole-4- carboxamide; 2,2,2-trifluoroacetic acid |
| 275 | 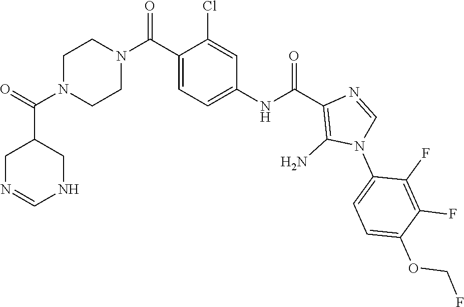 [SMILES: Nc1c(C(=O)Nc2ccc(C(=O)N3CCN(C(=O)C4CN=CNC4)CC3)c(Cl)c2)ncn1-c1ccc(OCF)c(F)c1F] | 5-amino-N-[3-chloro-4-[4-(1,4,5,6- tetrahydropyrimidine-5- carbonyl)piperazine-1-carbonyl]phenyl]- 1-[2,3-difluoro-4- (fluoromethoxy)phenyl]imidazole-4- carboxamide; bis(2,2,2-trifluoroacetic acid) |
| 276 | 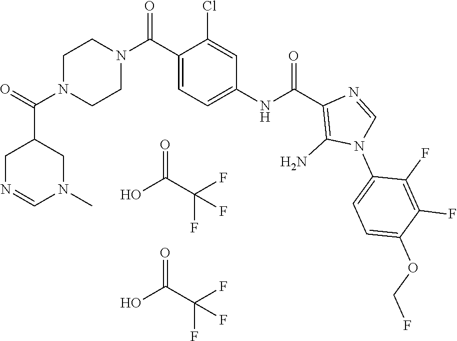 | 5-amino-N-[3-chloro-4-[4-(1-methyl-5,6- dihydro-4H-pyrimidine-5- carbonyl)piperazine-1-carbonyl]phenyl]- 1-[2,3-difluoro-4- (fluoromethoxy)phenyl]imidazole-4- carboxamide; bis(2,2,2-trifluoroacetic acid) |
| 277 | 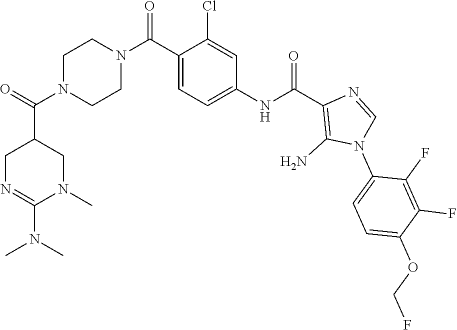 [SMILES: CN(C)C1=NCC(C(=O)N2CCN(C(=O)c3ccc(NC(=O)c4ncn(-c5ccc(OCF)c(F)c5F)c4N)cc3Cl)CC2)CN1C] | 5-amino-N-[3-chloro-4-[4-[2- (dimethylamino)-1-methyl-5,6-dihydro- 4H-pyrimidine-5-carbonyl]piperazine-1- carbonyl]phenyl]-1-[2,3-difluoro-4- (fluoromethoxy)phenyl]imidazole-4- carboxamide |
| 278 | 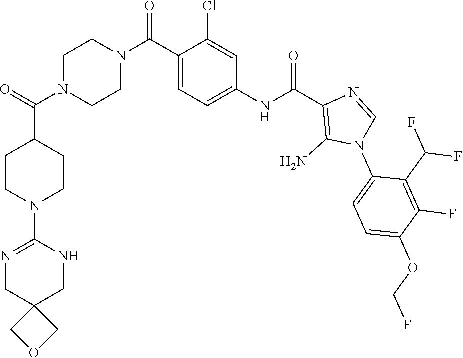 [SMILES: Nc1c(C(=O)Nc2ccc(C(=O)N3CCN(C(=O)C4CCN(C5=NCC6(CN5)COC6)CC4)CC3)c(Cl)c2)ncn1-c1ccc(OCF)c(F)c1C(F)F] | 5-amino-N-[3-chloro-4-[4-[1-(2-oxa-6,8- diazaspiro[3.5]non-6-en-7-yl)piperidine- 4-carbonyl]piperazine-1- carbonyl]phenyl]-1-[2-(difluoromethyl)-3- fluoro-4- (fluoromethoxy)phenyl]imidazole-4- carboxamide |
| 279 | 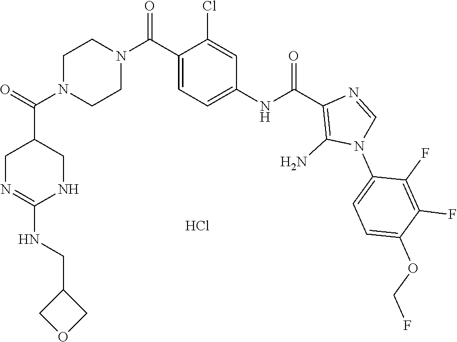 [SMILES: Nc1c(C(=O)Nc2ccc(C(=O)N3CCN(C(=O)C4CN=C(NCC5COC5)NC4)CC3)c(Cl)c2)ncn1-c1ccc(OCF)c(F)c1F ; Cl] | 5-amino-N-[3-chloro-4-[4-[2-(oxetan-3- ylmethylamino)-1,4,5,6- tetrahydropyrimidine-5- carbonyl]piperazine-1-carbonyl]phenyl]- 1-[2,3-difluoro-4- (fluoromethoxy)phenyl]imidazole-4- carboxamide; hydrochloride |
| 280 | 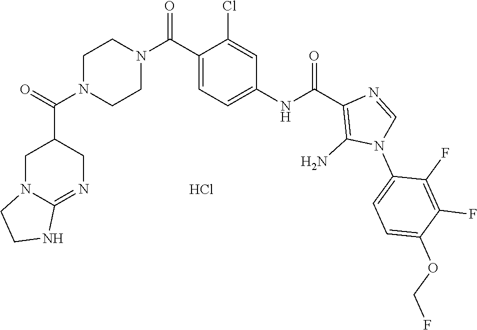 [SMILES: Nc1c(C(=O)Nc2ccc(C(=O)N3CCN(C(=O)C4CN=C5NCCN5C4)CC3)c(Cl)c2)ncn1-c1ccc(OCF)c(F)c1F ; Cl] | 5-amino-N-[3-chloro-4-[4-(1,2,3,5,6,7- hexahydroimidazo[1,2-a]pyrimidine-6- carbonyl)piperazine-1-carbonyl]phenyl]- 1-[2,3-difluoro-4- (fluoromethoxy)phenyl]imidazole-4- carboxamide; hydrochloride |
| 281 | 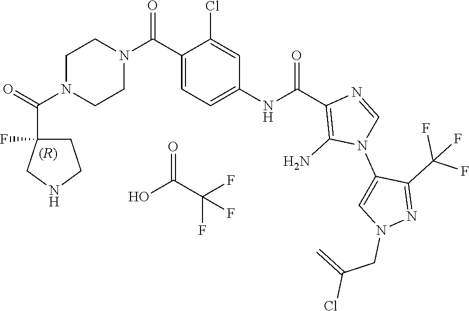 | 5-amino-1-[1-(2-chloroallyl)-3- (trifluoromethyl)pyrazol-4-yl]-N-[3- chloro-4-[4-[(3R)-3-fluoropyrrolidine-3- carbonyl]piperazine-1- carbonyl]phenyl]imidazole-4- carboxamide; 2,2,2-trifluoroacetic acid |
| 282 | 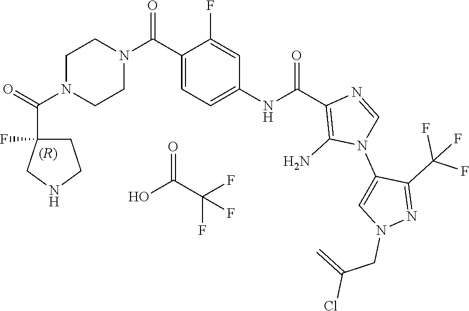 | 5-amino-1-[1-(2-chloroallyl)-3- (trifluoromethyl)pyrazol-4-yl]-N-[3-fluoro- 4-[4-[(3R)-3-fluoropyrrolidine-3- carbonyl]piperazine-1- carbonyl]phenyl]imidazole-4- carboxamide; 2,2,2-trifluoroacetic acid |
| 283 | 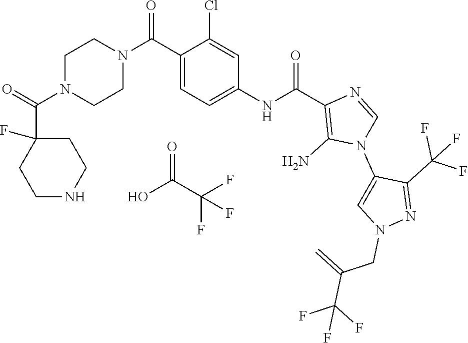 [SMILES: C=C(Cn1cc(-n2cnc(C(=O)Nc3ccc(C(=O)N4CCN(C(=O)C5(F)CCNCC5)CC4)c(Cl)c3)c2N)c(C(F)(F)F)n1)C(F)(F)F ; O=C(O)C(F)(F)F] | 5-amino-N-[3-chloro-4-[4-(4- fluoropiperidine-4-carbonyl)piperazine- 1-carbonyl]phenyl]-1-[3-(trifluoromethyl)- 1-[2-(trifluoromethyl)allyl]pyrazol-4- yllimidazole-4-carboxamide; 2,2,2- trifluoroacetic acid |
| 284 | 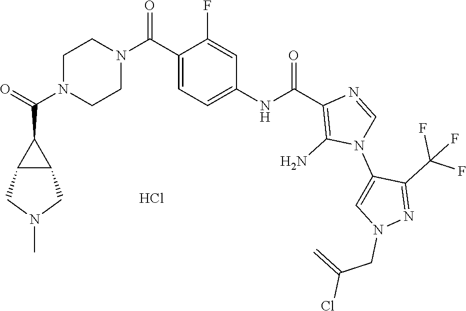 [SMILES: C=C(Cl)Cn1cc(-n2cnc(C(=O)Nc3ccc(C(=O)N4CCN(C(=O)[C@H]5[C@@H]6CN(C)C[C@@H]65)CC4)c(F)c3)c2N)c(C(F)(F)F)n1 ; Cl] | 5-amino-1-[1-(2-chloroallyl)-3- (trifluoromethyl)pyrazol-4-yl]-N-[3-fluoro- 4-[4-[(1S,5R)-3-methyl-3- azabicyclo[3.1.0]hexane-6- carbonyl]piperazine-1- carbonyl]phenyl]imidazole-4- carboxamide; hydrochloride |
| 285 | 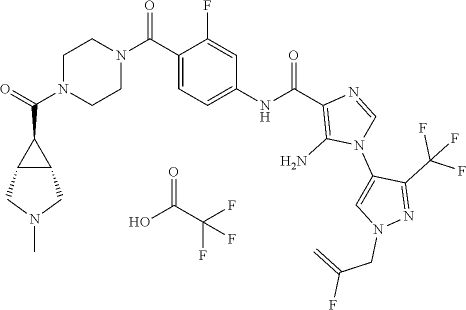 [SMILES: C=C(F)Cn1cc(-n2cnc(C(=O)Nc3ccc(C(=O)N4CCN(C(=O)[C@H]5[C@@H]6CN(C)C[C@@H]65)CC4)c(F)c3)c2N)c(C(F)(F)F)n1 ; O=C(O)C(F)(F)F] | 5-amino-1-[1-(2-fluoroallyl)-3- (trifluoromethyl)pyrazol-4-yl]-N-[3-fluoro- 4-[4-[(1S,5R)-3-methyl-3- azabicyclo[3.1.0]hexane-6- carbonyl]piperazine-1- carbonyl]phenyl]imidazole-4- carboxamide; 2,2,2-trifluoroacetic acid |
| 286 | 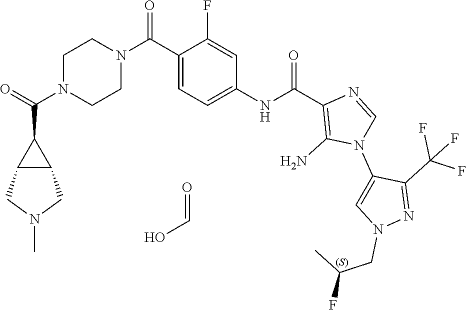 | 5-amino-N-[3-fluoro-4-[4-[(1S,5R)-3- methyl-3-azabicyclo[3.1.0]hexane-6- carbonyl]piperazine-1-carbonyl]phenyl]- 1-[1-[(2S)-2-fluoropropyl]-3- (trifluoromethyl)pyrazol-4-yl]imidazole-4- carboxamide; formic acid |
| 287 | 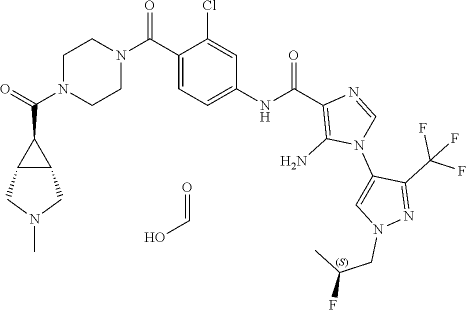 | 5-amino-N-[3-chloro-4-[4-[(1S,5R)-3- methyl-3-azabicyclo[3.1.0]hexane-6- carbonyl]piperazine-1-carbonyl]phenyl]- 1-[1-[(2S)-2-fluoropropyl]-3- (trifluoromethyl)pyrazol-4-yl]imidazole-4- carboxamide; formic acid |
| 288 | 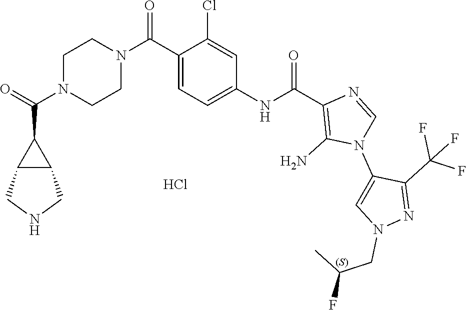 | 5-amino-N-[4-[4-[(1S,5R)-3- azabicyclo[3.1.0]hexane-6- carbonyl]piperazine-1-carbonyl]-3- chlorophenyl]-1-[1-[(2S)-2-fluoropropyl]- 3-(trifluoromethyl)pyrazol-4-yl]imidazole- 4-carboxamide; hydrochloride |
| 289 | 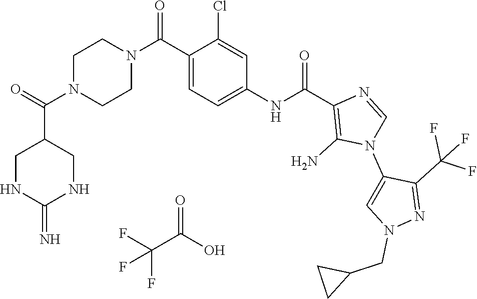 [SMILES: N=C1NCC(C(=O)N2CCN(C(=O)c3ccc(NC(=O)c4ncn(-c5cn(CC6CC6)nc5C(F)(F)F)c4N)cc3Cl)CC2)CN1 ; O=C(O)C(F)(F)F] | 5-amino-N-[3-chloro-4-[4-(2- iminohexahydropyrimidine-5- carbonyl)piperazine-1-carbonyl]phenyl]- 1-[1-(cyclopropylmethyl)-3- (trifluoromethyl)pyrazol-4-yl]imidazole-4- carboxamide; 2,2,2-trifluoroacetic acid |
| 290 | 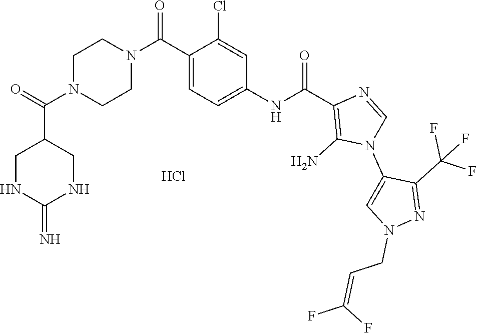 [SMILES: N=C1NCC(C(=O)N2CCN(C(=O)c3ccc(NC(=O)c4ncn(-c5cn(CC=C(F)F)nc5C(F)(F)F)c4N)cc3Cl)CC2)CN1 ; Cl] | 5-amino-N-[3-chloro-4-[4-(2- iminohexahydropyrimidine-5- carbonyl)piperazine-1-carbonyl]phenyl]- 1-[1-(3,3-difluoroallyl)-3- (trifluoromethyl)pyrazol-4-yl]imidazole-4- carboxamide; hydrochloride |
| 291 | 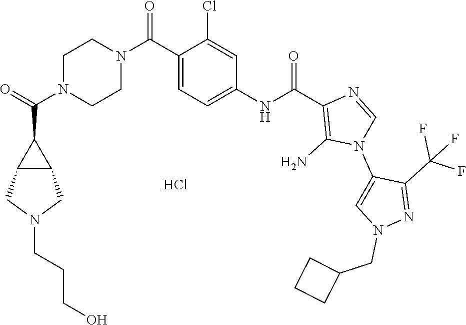 [SMILES: Nc1c(C(=O)Nc2ccc(C(=O)N3CCN(C(=O)[C@H]4[C@@H]5CN(CCCO)C[C@@H]54)CC3)c(Cl)c2)ncn1-c1cn(CC2CCC2)nc1C(F)(F)F ; Cl] | 5-amino-N-[3-chloro-4-[4-[rac-(1S,5R)- 3-(3-hydroxypropyl)-3- azabicyclo[3.1.0]hexane-6- carbonyl]piperazine-1-carbonyl]phenyl]- 1-[1-(cyclobutylmethyl)-3- (trifluoromethyl)pyrazol-4-yl]imidazole-4- carboxamide; hydrochloride |
| 292 | 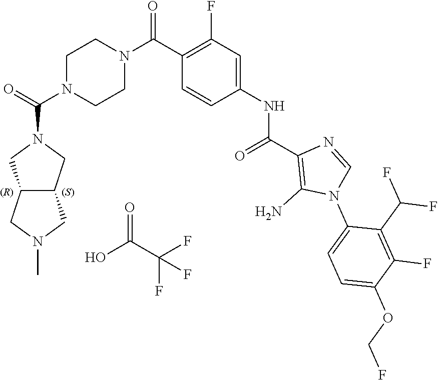 | N-[4-[4-[(3aS,6aR)-2-methyl- 1,3,3a,4,6,6a-hexahydropyrrolo[3,4- c]pyrrole-5-carbonyl]piperazine-1- carbonyl]-3-fluorophenyl]-5-amino-1-[2- (difluoromethyl)-3-fluoro-4- (fluoromethoxy)phenyl]imidazole-4- carboxamide; 2,2,2-trifluoroacetic acid |
| 293 | 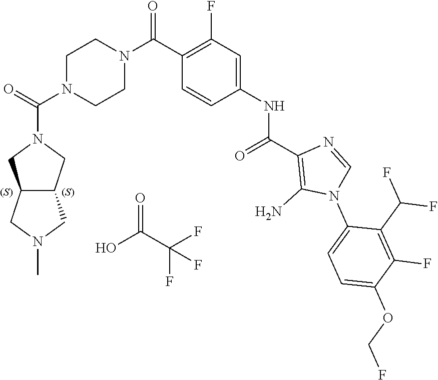 | N-[4-[4-[(3aS,6aS)-2-methyl- 1,3,3a,4,6,6a-hexahydropyrrolo[3,4- c]pyrrole-5-carbonyl]piperazine-1- carbonyl]-3-fluorophenyl]-5-amino-1-[2- (difluoromethyl)-3-fluoro-4- (fluoromethoxy)phenyl]imidazole-4- carboxamide; 2,2,2-trifluoroacetic acid |
| 294 | 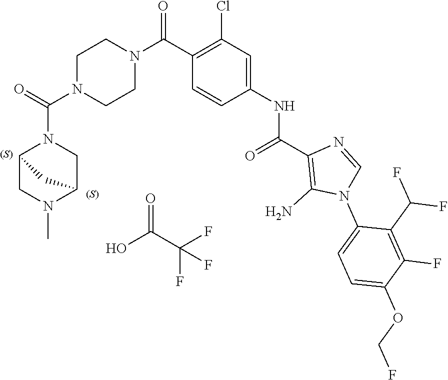 | 5-amino-N-[3-chloro-4-[4-[(1S,4S)-5- methyl-2,5-diazabicyclo[2.2.1]heptane- 2-carbonyl]piperazine-1- carbonyl]phenyl]-1-[2-(difluoromethyl)-3- fluoro-4- (fluoromethoxy)phenyl]imidazole-4- carboxamide; 2,2,2-trifluoroacetic acid |
| 295 | 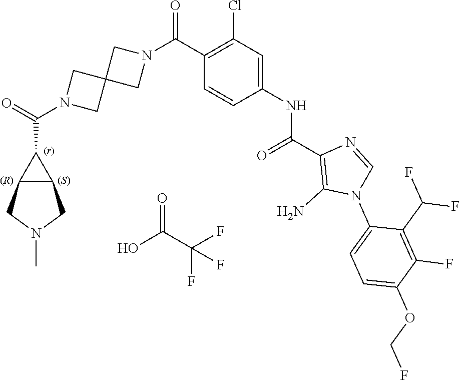 | 5-amino-N-[3-chloro-4-[2-[(1R,5S)-3- methyl-3-azabicyclo[3.1.0]hexane-6- carbonyl]-2,6-diazaspiro[3.3]heptane-6- carbonyl]phenyl]-1-[2-(difluoromethyl)-3- fluoro-4- (fluoromethoxy)phenyl]imidazole-4- carboxamide; 2,2,2-trifluoroacetic acid |
| 296 | 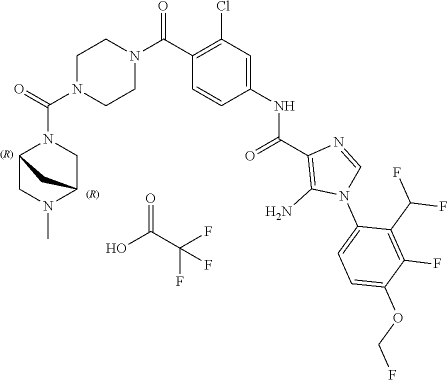 | 5-amino-N-[3-chloro-4-[4-[(1R,4R)-5- methyl-2,5-diazabicyclo[2.2.1]heptane- 2-carbonyl]piperazine-1- carbonyl]phenyl]-1-[2-(difluoromethyl)-3- fluoro-4- (fluoromethoxy)phenyl]imidazole-4- carboxamide; 2,2,2-trifluoroacetic acid |
| 297 | 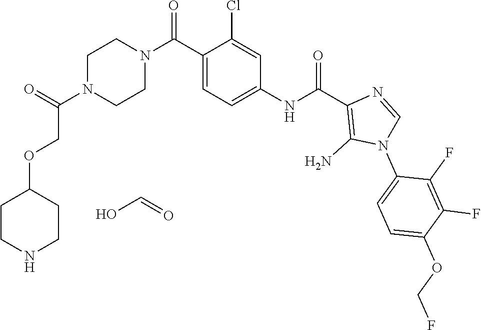 [SMILES: Nc1c(C(=O)Nc2ccc(C(=O)N3CCN(C(=O)COC4CCNCC4)CC3)c(Cl)c2)ncn1-c1ccc(OCF)c(F)c1F ; O=CO] | 5-amino-N-[3-chloro-4-[4-[2-(4- piperidyloxy)acetyl]piperazine-1- carbonyl]phenyl]-1-[2,3-difluoro-4- (fluoromethoxy)phenyl]imidazole-4- carboxamide; formic acid |
| 298 | 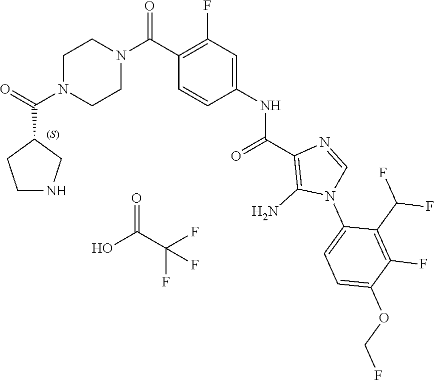 | 5-amino-1-[2-(difluoromethyl)-3-fluoro-4- (fluoromethoxy)phenyl]-N-[3-fluoro-4-[4- [(3S)-pyrrolidine-3-carbonyl]piperazine- 1-carbonyl]phenyl]imidazole-4- carboxamide; 2,2,2-trifluoroacetic acid |
| 299 | 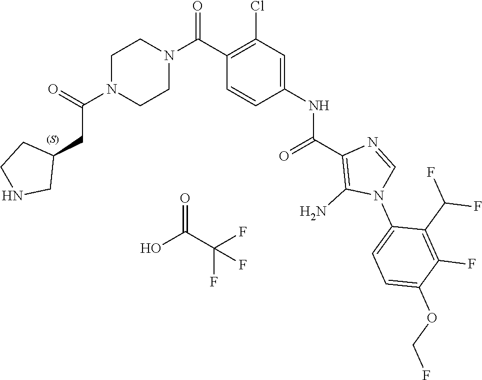 | 5-amino-N-[3-chloro-4-[4-[2-[(3R)- pyrrolidin-3-yl]acetyl]piperazine-1- carbonyl]phenyl]-1-[2-(difluoromethyl)-3- fluoro-4- (fluoromethoxy)phenyl]imidazole-4- carboxamide; 2,2,2-trifluoroacetic acid |
| 300 | 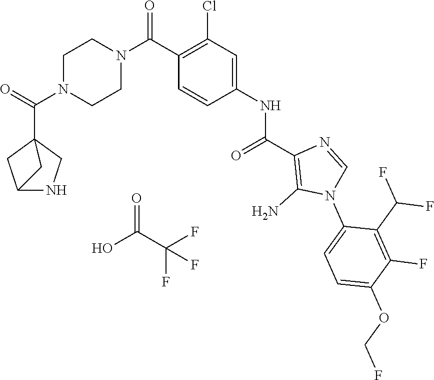 [SMILES: Nc1c(C(=O)Nc2ccc(C(=O)N3CCN(C(=O)C45CNC(C4)C5)CC3)c(Cl)c2)ncn1-c1ccc(OCF)c(F)c1C(F)F ; O=C(O)C(F)(F)F] | 5-amino-N-[4-[4-(2- azabicyclo[2.1.1 ]hexane-4- carbonyl)piperazine-1-carbonyl]-3- chlorophenyl]-1-[2-(difluoromethyl)-3- fluoro-4- (fluoromethoxy)phenyl]imidazole-4- carboxamide; 2,2,2-trifluoroacetic acid |
| 301 | 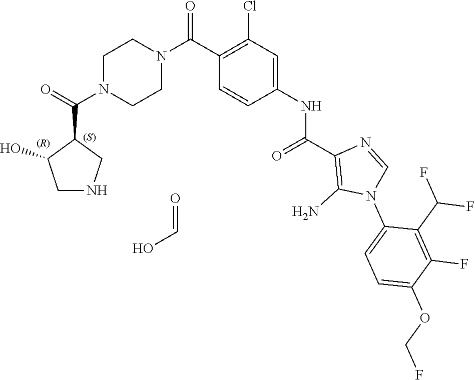 | 5-amino-N-[3-chloro-4-[4-[(3S,4R)-4- hydroxypyrrolidine-3- carbonyl]piperazine-1-carbonyl]phenyl]- 1-[2-(difluoromethyl)-3-fluoro-4- (fluoromethoxy)phenyl]imidazole-4- carboxamide; formic acid |
| 302 | 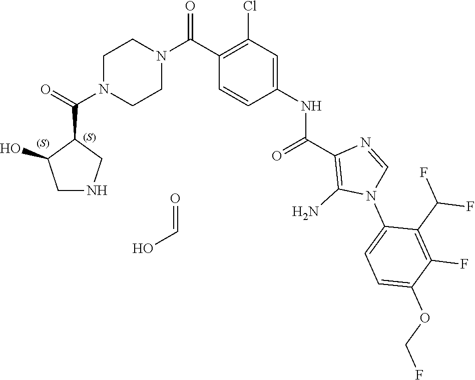 | 5-amino-N-[3-chloro-4-[4-[(3S,4S)-4- hydroxypyrrolidine-3- carbonyl]piperazine-1-carbonyl]phenyl]- 1-[2-(difluoromethyl)-3-fluoro-4- (fluoromethoxy)phenyl]imidazole-4- carboxamide; formic acid |
| 303 | 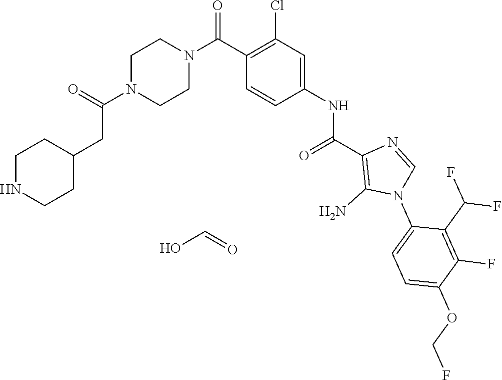 [SMILES: Nc1c(C(=O)Nc2ccc(C(=O)N3CCN(C(=O)CC4CCNCC4)CC3)c(Cl)c2)ncn1-c1ccc(OCF)c(F)c1C(F)F ; O=CO] | 5-amino-N-[3-chloro-4-[4-[2-(4- piperidyl)acetyl]piperazine-1- carbonyl]phenyl]-1-[2-(difluoromethyl)-3- fluoro-4- (fluoromethoxy)phenyl]imidazole-4- carboxamide; formic acid |
| 304 | 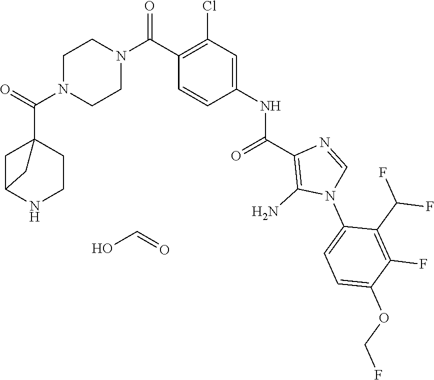 [SMILES: Nc1c(C(=O)Nc2ccc(C(=O)N3CCN(C(=O)C45CCNC(C4)C5)CC3)c(Cl)c2)ncn1-c1ccc(OCF)c(F)c1C(F)F ; O=CO] | 5-amino-N-[4-[4-(2- azabicyclo[3.1.1]heptane-5- carbonyl)piperazine-1-carbonyl]-3- chlorophenyl]-1-[2-(difluoromethyl)-3- fluoro-4- (fluoromethoxy)phenyl]imidazole-4- carboxamide; formic acid |
| 305 | 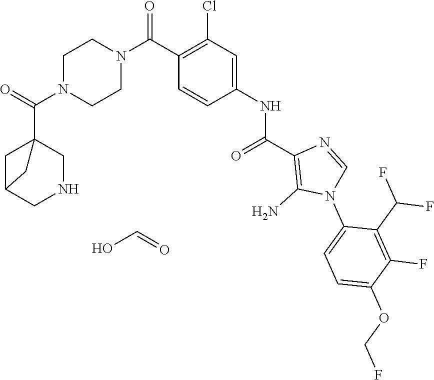 [SMILES: Nc1c(C(=O)Nc2ccc(C(=O)N3CCN(C(=O)C45CNCC(C4)C5)CC3)c(Cl)c2)ncn1-c1ccc(OCF)c(F)c1C(F)F ; O=CO] | 5-amino-N-[4-[4-(3- azabicyclo[3.1.1]heptane-1- carbonyl)piperazine-1-carbonyl]-3- chlorophenyl]-1-[2-(difluoromethyl)-3- fluoro-4- (fluoromethoxy)phenyl]imidazole-4- carboxamide; formic acid |
| 306 | 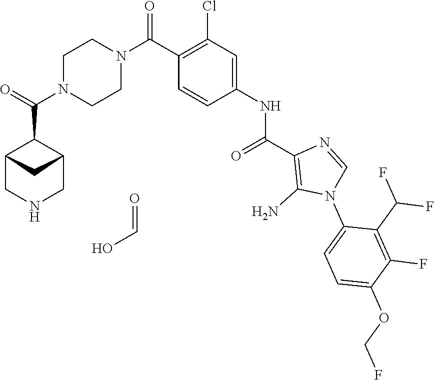 [SMILES: Nc1c(C(=O)Nc2ccc(C(=O)N3CCN(C(=O)[C@H]4[C@@H]5CNC[C@H]4C5)CC3)c(Cl)c2)ncn1-c1ccc(OCF)c(F)c1C(F)F ; O=CO] | 5-amino-N-[4-[4-[(1S,5R)-3- azabicyclo[3.1.1]heptane-6- carbonyl]piperazine-1-carbonyl]-3- chlorophenyl]-1-[2-(difluoromethyl)-3- fluoro-4- (fluoromethoxy)phenyl]imidazole-4- carboxamide; formic acid |
| 307 | 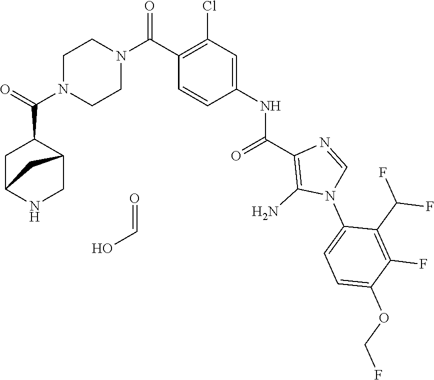 [SMILES: Nc1c(C(=O)Nc2ccc(C(=O)N3CCN(C(=O)[C@@H]4C[C@@H]5C[C@H]4CN5)CC3)c(Cl)c2)ncn1-c1ccc(OCF)c(F)c1C(F)F ; O=CO] | 5-amino-N-[4-[4-[(1S,4R,5R)-2- azabicyclo[2.2.1]heptane-5- carbonyl]piperazine-1-carbonyl]-3- chlorophenyl]-1-[2-(difluoromethyl)-3- fluoro-4- (fluoromethoxy)phenyl]imidazole-4- carboxamide; formic acid |
| 308 | 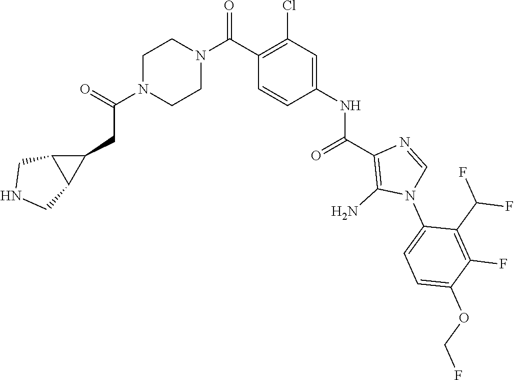 [SMILES: Nc1c(C(=O)Nc2ccc(C(=O)N3CCN(C(=O)C[C@H]4[C@@H]5CNC[C@@H]54)CC3)c(Cl)c2)ncn1-c1ccc(OCF)c(F)c1C(F)F] | 5-amino-N-[4-[4-[2-[(1R,5S)-3- azabicyclo[3.1.0]hexan-6- yl]acetyl]piperazine-1-carbonyl]-3- chlorophenyl]-1-[2-(difluoromethyl)-3- fluoro-4- (fluoromethoxy)phenyl]imidazole-4- carboxamide |
| 309 | 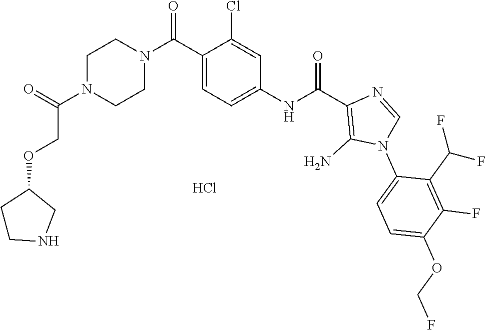 [SMILES: Nc1c(C(=O)Nc2ccc(C(=O)N3CCN(C(=O)CO[C@H]4CCNC4)CC3)c(Cl)c2)ncn1-c1ccc(OCF)c(F)c1C(F)F ; Cl] | 5-amino-N-[3-chloro-4-[4-[2-[(3S)- pyrrolidin-3-yl]oxyacetyl]piperazine-1- carbonyl]phenyl]-1-[2-(difluoromethyl)-3- fluoro-4- (fluoromethoxy)phenyl]imidazole-4- carboxamide; hydrochloride |
| 310 | 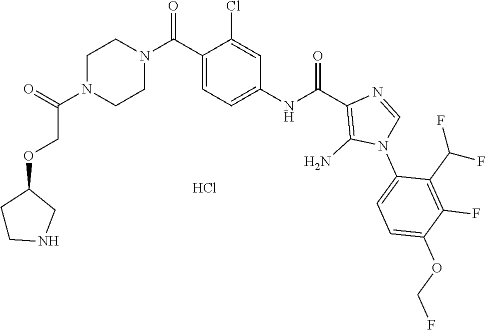 [SMILES: Nc1c(C(=O)Nc2ccc(C(=O)N3CCN(C(=O)CO[C@@H]4CCNC4)CC3)c(Cl)c2)ncn1-c1ccc(OCF)c(F)c1C(F)F ; Cl] | 5-amino-N-[3-chloro-4-[4-[2-[(3R)- pyrrolidin-3-yl]oxyacetyl]piperazine-1- carbonyl]phenyl]-1-[2-(difluoromethyl)-3- fluoro-4- (fluoromethoxy)phenyl]imidazole-4- carboxamide; hydrochloride |
| 311 | 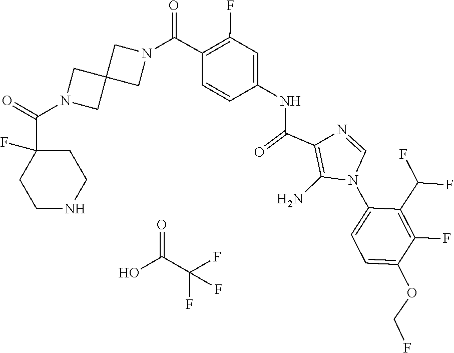 [SMILES: Nc1c(C(=O)Nc2ccc(C(=O)N3CC4(C3)CN(C(=O)C3(F)CCNCC3)C4)c(F)c2)ncn1-c1ccc(OCF)c(F)c1C(F)F ; O=C(O)C(F)(F)F] | 5-amino-N-[3-chloro-4-[2-(4- fluoropiperidine-4-carbonyl)-2,6- diazaspiro[3.3]heptane-6- carbonyl]phenyl]-1-[2-(difluoromethyl)-3- fluoro-4- (fluoromethoxy)phenyl]imidazole-4- carboxamide; 2,2,2-trifluoroacetic acid |
| 312 | 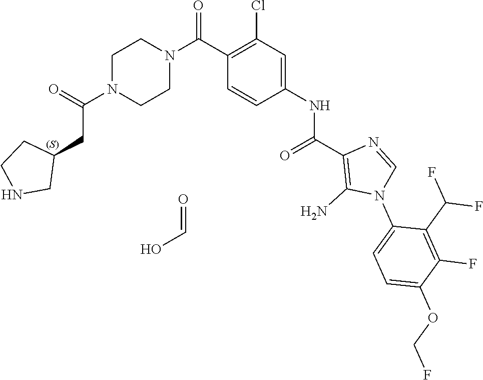 | 5-amino-N-[3-chloro-4-[4-[2-[(3S)- pyrrolidin-3-yl]acetyl]piperazine-1- carbonyl]phenyl]-1-[2,3-difluoro-4- (fluoromethoxy)phenyl]imidazole-4- carboxamide; formic acid |
| 313 | 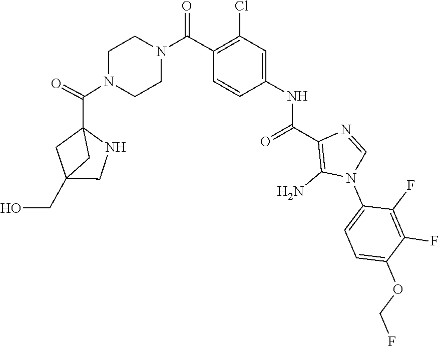 [SMILES: Nc1c(C(=O)Nc2ccc(C(=O)N3CCN(C(=O)C45CC(CO)(CN4)C5)CC3)c(Cl)c2)ncn1-c1ccc(OCF)c(F)c1F] | 5-amino-N-[3-chloro-4-[4-[4- (hydroxymethyl)-2- azabicyclo[2.1.1]hexane-1- carbonyl]piperazine-1-carbonyl]phenyl]- 1-[2,3-difluoro-4- (fluoromethoxy)phenyl]imidazole-4- carboxamide |
| 314 | 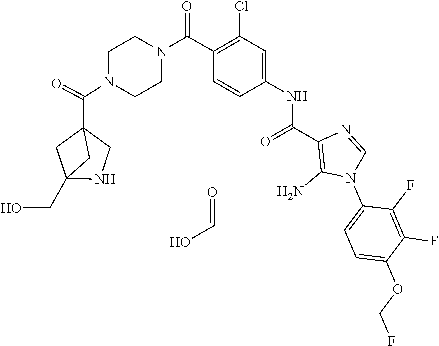 [SMILES: Nc1c(C(=O)Nc2ccc(C(=O)N3CCN(C(=O)C45CNC(CO)(C4)C5)CC3)c(Cl)c2)ncn1-c1ccc(OCF)c(F)c1F ; O=CO] | 5-amino-N-[3-chloro-4-[4-[1- (hydroxymethyl)-2- azabicyclo[2.1.1]hexane-4- carbonyl]piperazine-1-carbonyl]phenyl]- 1-[2,3-difluoro-4- (fluoromethoxy)phenyl]imidazole-4- carboxamide; formic acid |
| 315 | 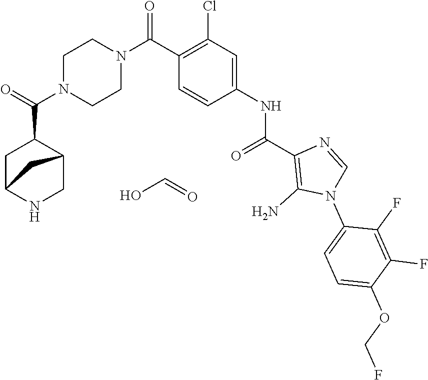 [SMILES: Nc1c(C(=O)Nc2ccc(C(=O)N3CCN(C(=O)[C@@H]4C[C@@H]5C[C@H]4CN5)CC3)c(Cl)c2)ncn1-c1ccc(OCF)c(F)c1F ; O=CO] | 5-amino-N-[4-[4-[(1S,4R,5R)-2- azabicyclo[2.2.1]heptane-5- carbonyl]piperazine-1-carbonyl]-3- chlorophenyl]-1-[2,3-difluoro-4- (fluoromethoxy)phenyl]imidazole-4- carboxamide; formic acid |
| 316 | 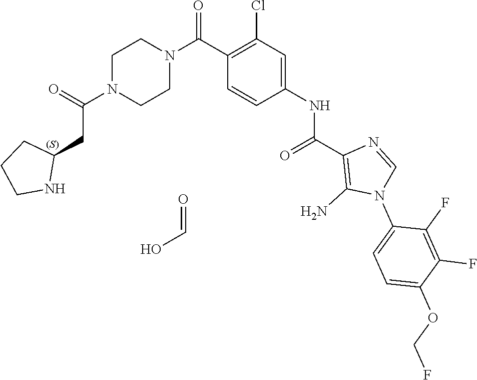 | 5-amino-N-[3-chloro-4-[4-[2-[(2S)- pyrrolidin-2-yl]acetyl]piperazine-1- carbonyl]phenyl]-1-[2,3-difluoro-4- (fluoromethoxy)phenyl]imidazole-4- carboxamide; formic acid |
| 317 | 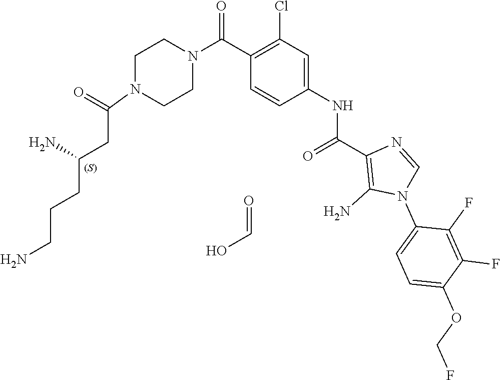 | 5-amino-N-[3-chloro-4-[4-[(3S)-3,6- diaminohexanoyl]piperazine-1- carbonyl]phenyl]-1-[2,3-difluoro-4- (fluoromethoxy)phenyl]imidazole-4- carboxamide; formic acid |
| 318 | 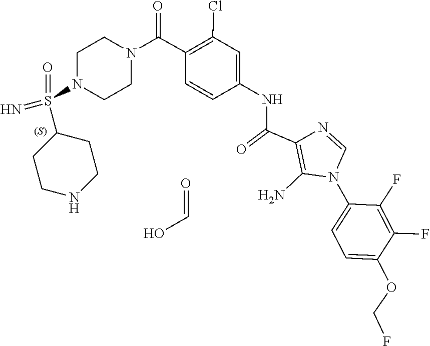 | (S)-5-amino-N-(3-chloro-4-(4- (piperidine-4-sulfonimidoyl)piperidine-1- carbonyl)phenyl)-1-(2,3-difluoro-4- (fluoromethoxy)phenyl)-1H-imidazole-4- carboxamide; formic acid |
| 319 | 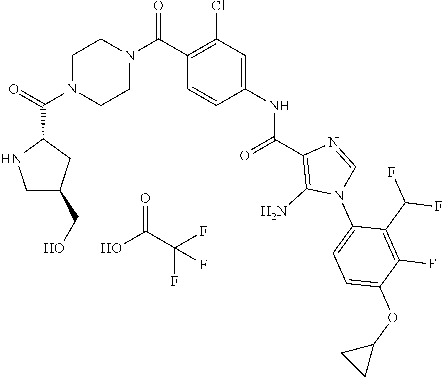 [SMILES: Nc1c(C(=O)Nc2ccc(C(=O)N3CCN(C(=O)[C@@H]4C[C@@H](CO)CN4)CC3)c(Cl)c2)ncn1-c1ccc(OC2CC2)c(F)c1C(F)F ; O=C(O)C(F)(F)F] | 5-amino-N-[3-chloro-4-[4-[(2S,4R)-4- (hydroxymethyl)pyrrolidine-2- carbonyl]piperazine-1-carbonyl]phenyl]- 1-[4-(cyclopropoxy)-2-(difluoromethyl)- 3-fluorophenyl]imidazole-4- carboxamide; 2,2,2-trifluoroacetic acid |
| 320 | 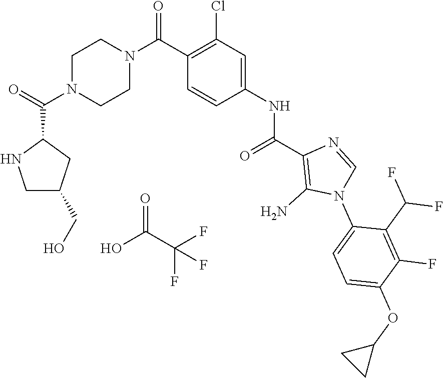 [SMILES: Nc1c(C(=O)Nc2ccc(C(=O)N3CCN(C(=O)[C@@H]4C[C@H](CO)CN4)CC3)c(Cl)c2)ncn1-c1ccc(OC2CC2)c(F)c1C(F)F ; O=C(O)C(F)(F)F] | 5-amino-N-[3-chloro-4-[4-[(2S,4S)-4- (hydroxymethyl)pyrrolidine-2- carbonyl]piperazine-1-carbonyl]phenyl]- 1-[4-(cyclopropoxy)-2-(difluoromethyl)- 3-fluorophenyl]imidazole-4- carboxamide; 2,2,2-trifluoroacetic acid |
| 321 | 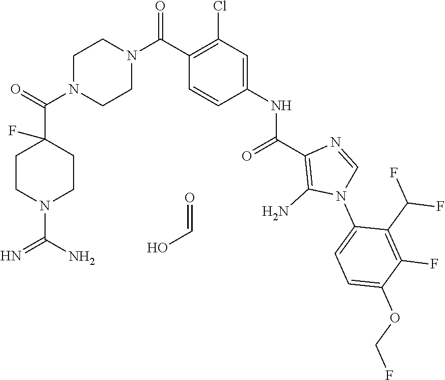 [SMILES: N=C(N)N1CCC(F)(C(=O)N2CCN(C(=O)c3ccc(NC(=O)c4ncn(-c5ccc(OCF)c(F)c5C(F)F)c4N)cc3Cl)CC2)CC1 ; O=CO] | 5-amino-N-[4-[4-(1-carbamimidoyl-4- fluoro-piperidine-4-carbonyl)piperazine- 1-carbonyl]-3-chlorophenyl]-1-[2- (difluoromethyl)-3-fluoro-4- (fluoromethoxy)phenyl]imidazole-4- carboxamide; formic acid |
| 322 | 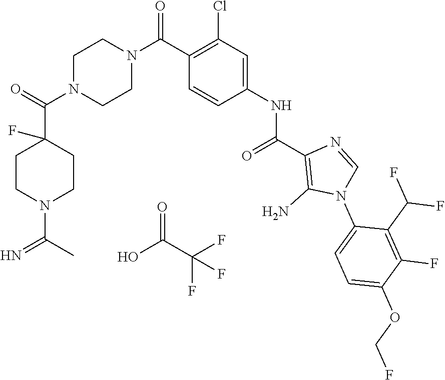 [SMILES: CC(=N)N1CCC(F)(C(=O)N2CCN(C(=O)c3ccc(NC(=O)c4ncn(-c5ccc(OCF)c(F)c5C(F)F)c4N)cc3Cl)CC2)CC1 ; O=C(O)C(F)(F)F] | 5-amino-N-[3-chloro-4-[4-(1- ethanimidoyl-4-fluoro-piperidine-4- carbonyl)piperazine-1-carbonyl]phenyl]- 1-[2-(difluoromethyl)-3-fluoro-4- (fluoromethoxy)phenyl]imidazole-4- carboxamide; 2,2,2-trifluoroacetic acid |
| 323 | 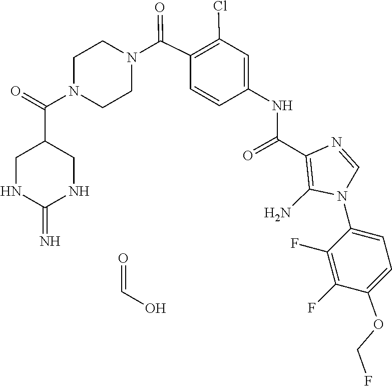 [SMILES: N=C1NCC(C(=O)N2CCN(C(=O)c3ccc(NC(=O)c4ncn(-c5ccc(OCF)c(F)c5F)c4N)cc3Cl)CC2)CN1 ; O=CO] | 5-amino-N-[3-chloro-4-[4-(2- iminohexahydropyrimidine-5- carbonyl)piperazine-1-carbonyl]phenyl]- 1-[2,3-difluoro-4- (fluoromethoxy)phenyl]imidazole-4- carboxamide; formic acid |
| 324 | 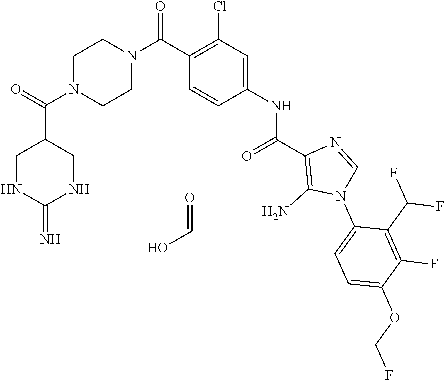 [SMILES: N=C1NCC(C(=O)N2CCN(C(=O)c3ccc(NC(=O)c4ncn(-c5ccc(OCF)c(F)c5C(F)F)c4N)cc3Cl)CC2)CN1 ; O=CO] | 5-amino-N-[3-chloro-4-[4-(2- iminohexahydropyrimidine-5- carbonyl)piperazine-1-carbonyl]phenyl]- 1-[2-(difluoromethyl)-3-fluoro-4- (fluoromethoxy)phenyl]imidazole-4- carboxamide; formic acid |
| 325 | 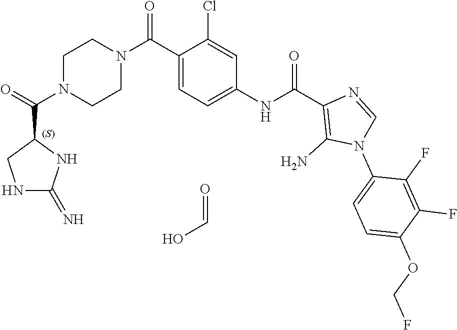 | 5-amino-N-[3-chloro-4-[4-[(4S)-2- iminoimidazolidine-4- carbonyl]piperazine-1-carbonyl]phenyl]- 1-[2-(difluoromethyl)-3-fluoro-4- (fluoromethoxy)phenyl]imidazole-4- carboxamide; formic acid |
| 326 | 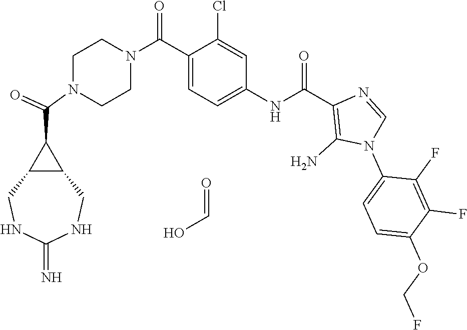 [SMILES: N=C1NC[C@@H]2[C@H](CN1)[C@H]2C(=O)N1CCN(C(=O)c2ccc(NC(=O)c3ncn(-c4ccc(OCF)c(F)c4F)c3N)cc2Cl)CC1 ; O=CO] | 5-amino-N-[3-chloro-4-[4-[(1S,7R)-4- imino-3,5-diazabicyclo[5.1.0]octane-8- carbonyl]piperazine-1-carbonyl]phenyl]- 1-[2,3-difluoro-4- (fluoromethoxy)phenyl]imidazole-4- carboxamide; formic acid |
| 327 | 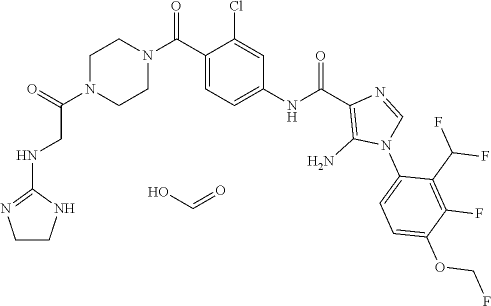 [SMILES: Nc1c(C(=O)Nc2ccc(C(=O)N3CCN(C(=O)CNC4=NCCN4)CC3)c(Cl)c2)ncn1-c1ccc(OCF)c(F)c1C(F)F ; O=CO] | 5-amino-N-[3-chloro-4-[4-[2-(4,5- dihydro-1H-imidazol-2- ylamino)acetyl]piperazine-1- carbonyl]phenyl]-1-[2-(difluoromethyl)-3- fluoro-4- (fluoromethoxy)phenyl]imidazole-4- carboxamide; formic acid |
| 328 | 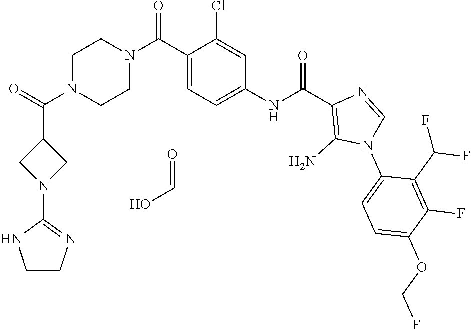 [SMILES: Nc1c(C(=O)Nc2ccc(C(=O)N3CCN(C(=O)C4CN(C5=NCCN5)C4)CC3)c(Cl)c2)ncn1-c1ccc(OCF)c(F)c1C(F)F ; O=CO] | 5-amino-N-[3-chloro-4-[4-[1-(4,5- dihydro-1H-imidazol-2-yl)azetidine-3- carbonyl]piperazine-1-carbonyl]phenyl]- 1-[2-(difluoromethyl)-3-fluoro-4- (fluoromethoxy)phenyl]imidazole-4- carboxamide; formic acid |
| 329 | 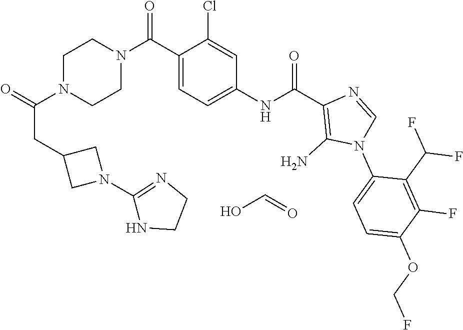 [SMILES: Nc1c(C(=O)Nc2ccc(C(=O)N3CCN(C(=O)CC4CN(C5=NCCN5)C4)CC3)c(Cl)c2)ncn1-c1ccc(OCF)c(F)c1C(F)F ; O=CO] | 5-amino-N-[3-chloro-4-[4-[2-[1-(4,5- dihydro-1H-imidazol-2-yl)azetidin-3- yl]acetyl]piperazine-1-carbonyl]phenyl]- 1-[2-(difluoromethyl)-3-fluoro-4- (fluoromethoxy)phenyl]imidazole-4- carboxamide; formic acid |
| 330 | 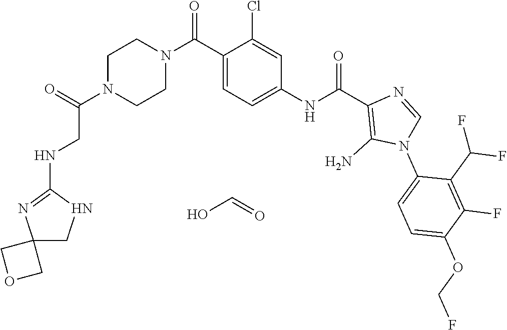 [SMILES: Nc1c(C(=O)Nc2ccc(C(=O)N3CCN(C(=O)CNC4=NC5(CN4)COC5)CC3)c(Cl)c2)ncn1-c1ccc(OCF)c(F)c1C(F)F ; O=CO] | 5-amino-N-[3-chloro-4-[4-[2-(2-oxa-5,7- diazaspiro[3.4]oct-5-en-6- ylamino)acetyl]piperazine-1- carbonyl]phenyl]-1-[2-(difluoromethyl)-3- fluoro-4- (fluoromethoxy)phenyl]imidazole-4- carboxamide; formic acid |
| 331 | 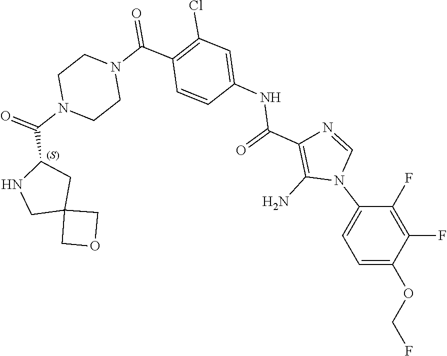 | 5-amino-N-[3-chloro-4-[4-[(6S)-2-oxa-7- azaspiro[3.4]octane-6- carbonyl]piperazine-1-carbonyl]phenyl]- 1-[2,3-difluoro-4- (fluoromethoxy)phenyl]imidazole-4- carboxamide |
| 332 | 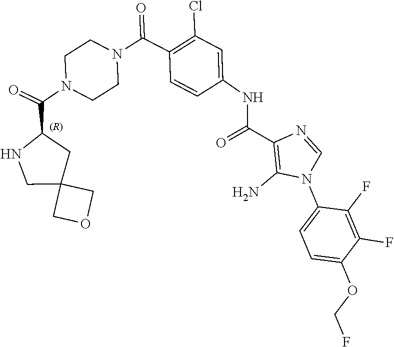 | 5-amino-N-[3-chloro-4-[4-[(6R)-2-oxa-7- azaspiro[3.4]octane-6- carbonyl]piperazine-1-carbonyl]phenyl]- 1-[2,3-difluoro-4- (fluoromethoxy)phenyl]imidazole-4- carboxamide |
| 333 | 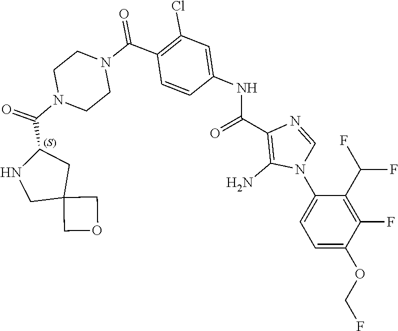 | 5-amino-N-[3-chloro-4-[4-[(6S)-2-oxa-7- azaspiro[3.4]octane-6- carbonyl]piperazine-1-carbonyl]phenyl]- 1-[2-(difluoromethyl)-3-fluoro-4- (fluoromethoxy)phenyl]imidazole-4- carboxamide |
| 334 | 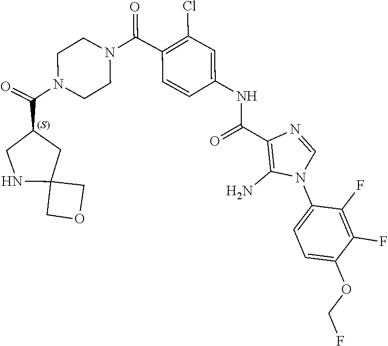 | 5-amino-N-[3-chloro-4-[4-[(7S)-2-oxa-5- azaspiro[3.4]octane-7- carbonyl]piperazine-1-carbonyl]phenyl]- 1-[2,3-difluoro-4- (fluoromethoxy)phenyl]imidazole-4- carboxamide |
| 335 | 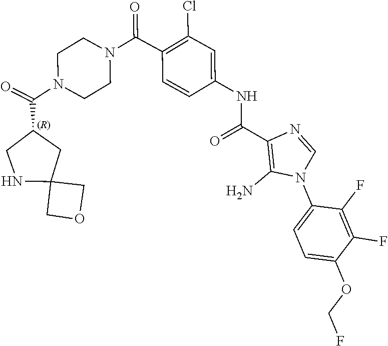 | 5-amino-N-[3-chloro-4-[4-[(7R)-2-oxa-5- azaspiro[3.4]octane-7- carbonyl]piperazine-1-carbonyl]phenyl]- 1-[2,3-difluoro-4- (fluoromethoxy)phenyl]imidazole-4- carboxamide |
| 336 | 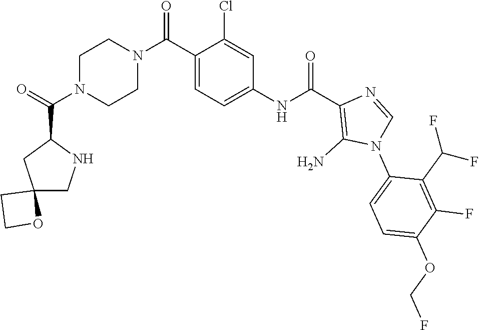 [SMILES: Nc1c(C(=O)Nc2ccc(C(=O)N3CCN(C(=O)[C@@H]4C[C@@]5(CCO5)CN4)CC3)c(Cl)c2)ncn1-c1ccc(OCF)c(F)c1C(F)F] | 5-amino-N-[3-chloro-4-[4-[(4R,6S)-1- oxa-7-azaspiro[3.4]octane-6- carbonyl]piperazine-1-carbonyl]phenyl]- 1-[2-(difluoromethyl)-3-fluoro-4- (fluoromethoxy)phenyl]imidazole-4- carboxamide |
| 337 | 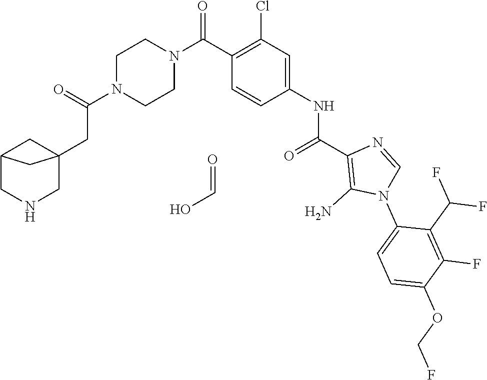 [SMILES: Nc1c(C(=O)Nc2ccc(C(=O)N3CCN(C(=O)CC45CNCC(C4)C5)CC3)c(Cl)c2)ncn1-c1ccc(OCF)c(F)c1C(F)F ; O=CO] | 5-amino-N-[4-[4-[2-(3- azabicyclo[3.1.1]heptan-1- yl)acetyl]piperazine-1-carbonyl]-3- chlorophenyl]-1-[2-(difluoromethyl)-3- fluoro-4- (fluoromethoxy)phenyl]imidazole-4- carboxamide; formic acid |
| 338 | 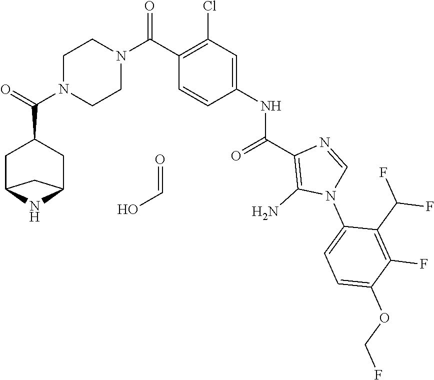 [SMILES: Nc1c(C(=O)Nc2ccc(C(=O)N3CCN(C(=O)[C@@H]4C[C@H]5C[C@@H](C4)N5)CC3)c(Cl)c2)ncn1-c1ccc(OCF)c(F)c1C(F)F ; O=CO] | 5-amino-N-[3-chloro-4-[4-[rac-(1S,5R)- 6-azabicyclo[3.1.1]heptane-3- carbonyl]piperazine-1-carbonyl]phenyl]- 1-[2-(difluoromethyl)-3-fluoro-4- (fluoromethoxy)phenyl]imidazole-4- carboxamide; formic acid |
| 339 | 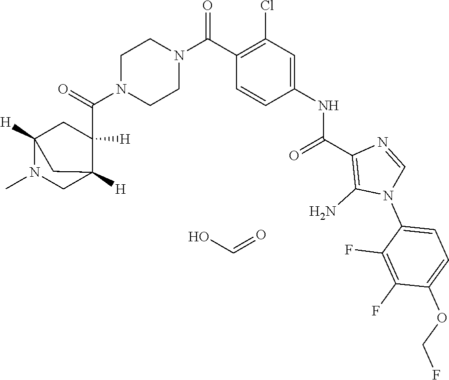 [SMILES: CN1C[C@@H]2C[C@H]1C[C@H]2C(=O)N1CCN(C(=O)c2ccc(NC(=O)c3ncn(-c4ccc(OCF)c(F)c4F)c3N)cc2Cl)CC1 ; O=CO] | 5-amino-N-[3-chloro-4-[4-[(1S,4R,5R)-2- methyl-2-azabicyclo[2.2.1]heptane-5- carbonyl]piperazine-1-carbonyl]phenyl]- 1-[2,3-difluoro-4- (fluoromethoxy)phenyl]imidazole-4- carboxamide; formic acid |
| 340 | 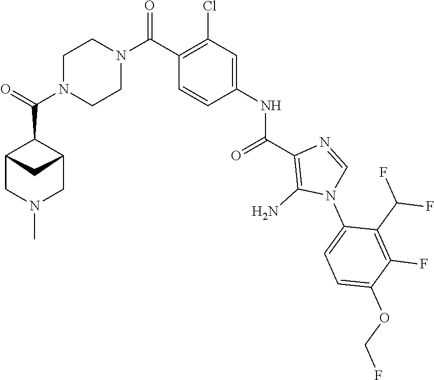 [SMILES: CN1C[C@H]2C[C@@H](C1)[C@@H]2C(=O)N1CCN(C(=O)c2ccc(NC(=O)c3ncn(-c4ccc(OCF)c(F)c4C(F)F)c3N)cc2Cl)CC1] | 5-amino-N-[3-chloro-4-[4-[(1S,5R)-3- methyl-3-azabicyclo[3.1.1]heptane-6- carbonyl]piperazine-1-carbonyl]phenyl]- 1-[2-(difluoromethyl)-3-fluoro-4- (fluoromethoxy)phenyl]imidazole-4- carboxamide |
| 341 | 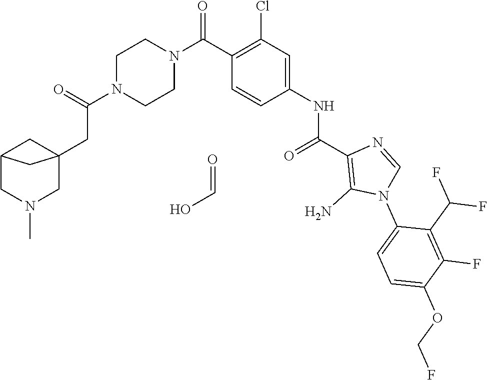 [SMILES: CN1CC2CC(CC(=O)N3CCN(C(=O)c4ccc(NC(=O)c5ncn(-c6ccc(OCF)c(F)c6C(F)F)c5N)cc4Cl)CC3)(C2)C1 ; O=CO] | 5-amino-N-[3-chloro-4-[4-[2-(3-methyl-3- azabicyclo[3.1.1]heptan-1- yl)acetyl]piperazine-1-carbonyl]phenyl]- 1-[2-(difluoromethyl)-3-fluoro-4- (fluoromethoxy)phenyl]imidazole-4- carboxamide; formic acid |
| 342 | 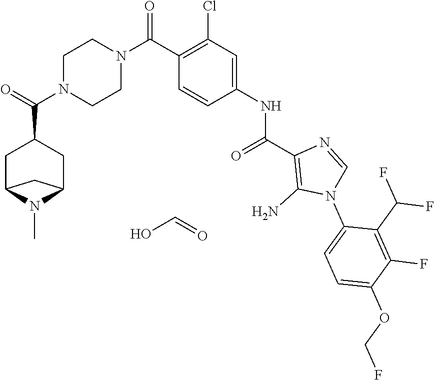 [SMILES: CN1[C@@H]2C[C@H](C(=O)N3CCN(C(=O)c4ccc(NC(=O)c5ncn(-c6ccc(OCF)c(F)c6C(F)F)c5N)cc4Cl)CC3)C[C@H]1C2 ; O=CO] | 5-amino-N-[3-chloro-4-[4-[(1S,5R)-6- methyl-6-azabicyclo[3.1.1]heptane-3- carbonyl]piperazine-1-carbonyl]phenyl]- 1-[2-(difluoromethyl)-3-fluoro-4- (fluoromethoxy)phenyl]imidazole-4- carboxamide; formic acid |
| 343 | 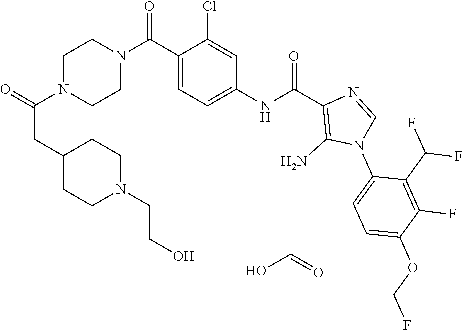 [SMILES: Nc1c(C(=O)Nc2ccc(C(=O)N3CCN(C(=O)CC4CCN(CCO)CC4)CC3)c(Cl)c2)ncn1-c1ccc(OCF)c(F)c1C(F)F ; O=CO] | 5-amino-N-[3-chloro-4-[4-[2-[1-(2- hydroxyethyl)-4- piperidyl]acetyl]piperazine-1- carbonyl]phenyl]-1-[2-(difluoromethyl)-3- fluoro-4- (fluoromethoxy)phenyl]imidazole-4- carboxamide; formic acid |
| 344 | 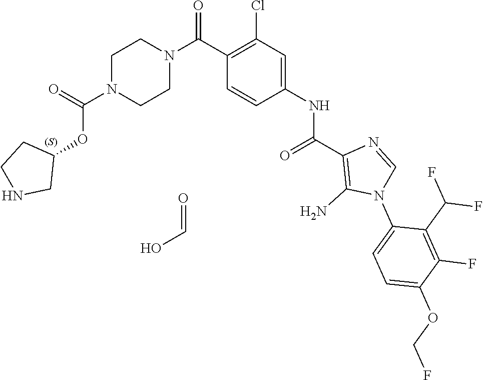 | [(3S)-pyrrolidin-3-yl] 4-[4-[[5-amino-1-[2- (difluoromethyl)-3-fluoro-4- (fluoromethoxy)phenyl]imidazole-4- carbonyl]amino]-2-chloro- benzoyl]piperazine-1-carboxylate; formic acid |
| 345 | 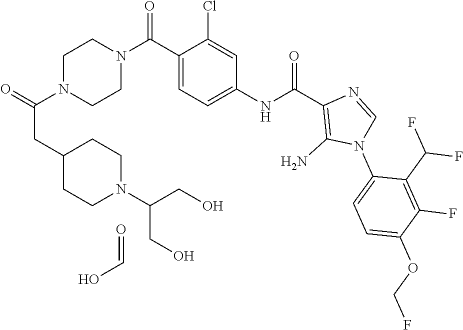 | 5-amino-N-[3-chloro-4-[4-[2-[1-[2- hydroxy-1 -(hydroxymethyl)ethyl]-4- piperidyl]acetyl]piperazine-1- carbonyl]phenyl]-1-[2-(difluoromethyl)-3- fluoro-4- (fluoromethoxy)phenyl]imidazole-4- carboxamide; formic acid |
| 346 | 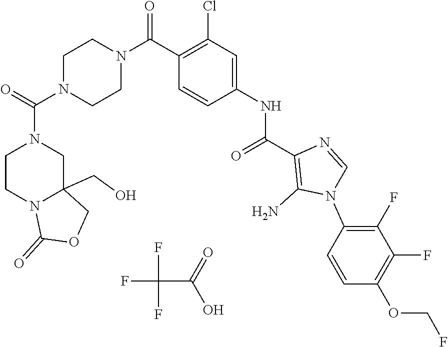 [SMILES: Nc1c(C(=O)Nc2ccc(C(=O)N3CCN(C(=O)N4CCN5C(=O)OCC5(CO)C4)CC3)c(Cl)c2)ncn1-c1ccc(OCF)c(F)c1F ; O=C(O)C(F)(F)F] | 5-amino-N-[3-chloro-4-[4-(3-keto-8a- methylol-1,5,6,8-tetrahydrooxazolo[3,4- alpyrazine-7-carbonyl)piperazine-1- carbonyl]phenyl]-1-[2,3-difluoro-4- (fluoromethoxy)phenyl]imidazole-4- carboxamide. 1:.63 2,2,2-trifluoroacetic acid |
| 347 | 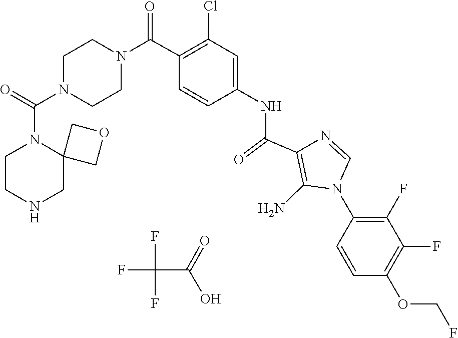 [SMILES: Nc1c(C(=O)Nc2ccc(C(=O)N3CCN(C(=O)N4CCNCC45COC5)CC3)c(Cl)c2)ncn1-c1ccc(OCF)c(F)c1F ; O=C(O)C(F)(F)F] | N-(4-(4-(2-oxa-5,8- diazaspiro[3.5]nonane-5- carbonyl)piperazine-1-carbonyl)-3- chlorophenyl)-5-amino-1-(2,3-difluoro-4- (fluoromethoxy)phenyl)-1H-imidazole-4- carboxamide 2,2,2-trifluoroacetate |
| 348 | 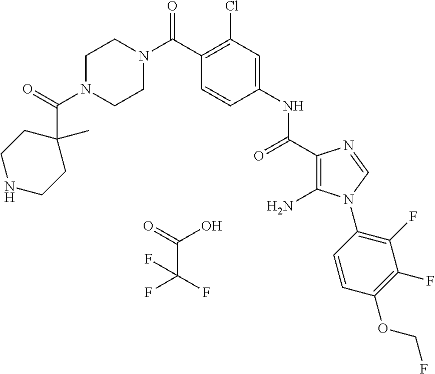 [SMILES: CC1(C(=O)N2CCN(C(=O)c3ccc(NC(=O)c4ncn(-c5ccc(OCF)c(F)c5F)c4N)cc3Cl)CC2)CCNCC1 ; O=C(O)C(F)(F)F] | 5-amino-N-[3-chloro-4-[4-(4- methylisonipecotoyl)piperazine-1- carbonyl]phenyl]-1-[2,3-difluoro-4- (fluoromethoxy)phenyl]imidazole-4- carboxamide. 1:1.3 2,2,2-trifluoroacetic acid |
| 349 | 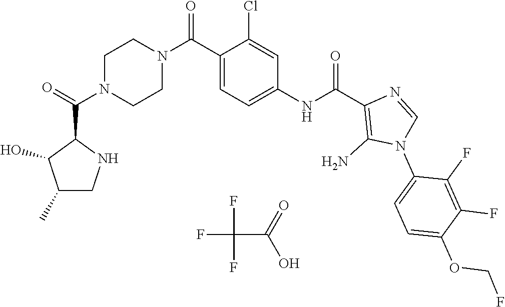 [SMILES: C[C@H]1CN[C@H](C(=O)N2CCN(C(=O)c3ccc(NC(=O)c4ncn(-c5ccc(OCF)c(F)c5F)c4N)cc3Cl)CC2)[C@H]1O ; O=C(O)C(F)(F)F] | 5-amino-N-[3-chloro-4-[4-[(2S,3S,4S)-3- hydroxy-4-methyl-prolyl]piperazine-1- carbonyl]phenyl]-1-[2,3-difluoro-4- (fluoromethoxy)phenyl]imidazole-4- carboxamide. 1:1 2,2,2-trifluoroacetic acid |
| 350 | 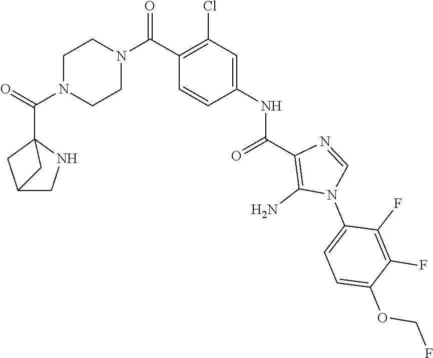 [SMILES: Nc1c(C(=O)Nc2ccc(C(=O)N3CCN(C(=O)C45CC(CN4)C5)CC3)c(Cl)c2)ncn1-c1ccc(OCF)c(F)c1F] | 5-amino-N-[4-[4-(2- azabicyclo[2.1.1]hexane-1- carbonyl)piperazine-1-carbonyl]-3- chlorophenyl]-1-[2,3-difluoro-4- (fluoromethoxy)phenyl]imidazole-4- carboxamide |
| 351 | 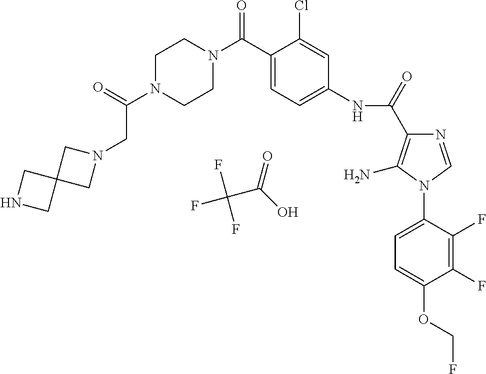 [SMILES: Nc1c(C(=O)Nc2ccc(C(=O)N3CCN(C(=O)CN4CC5(CNC5)C4)CC3)c(Cl)c2)ncn1-c1ccc(OCF)c(F)c1F ; O=C(O)C(F)(F)F] | 5-amino-N-[3-chloro-4-[4-[2-(2,6- diazaspiro[3.3]heptan-2- yl)acetyl]piperazine-1-carbonyl]phenyl]- 1-[2,3-difluoro-4- (fluoromethoxy)phenyl]imidazole-4- carboxamide. 1:2 2,2,2-trifluoroacetic acid |
| 352 | 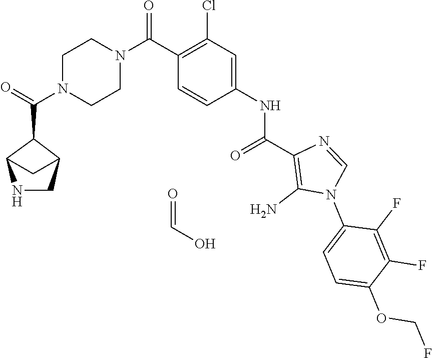 [SMILES: Nc1c(C(=O)Nc2ccc(C(=O)N3CCN(C(=O)[C@H]4[C@H]5CN[C@@H]4C5)CC3)c(Cl)c2)ncn1-c1ccc(OCF)c(F)c1F ; O=CO] | rac-5-amino-N-[4-[4-[(1S,4R,5R)-2- azabicyclo[2.1.1]hexane-5- carbonyl]piperazine-1-carbonyl]-3- chlorophenyl]-1-[2,3-difluoro-4- (fluoromethoxy)phenyl]imidazole-4- carboxamide. 1:1 formic acid |
| 353 | 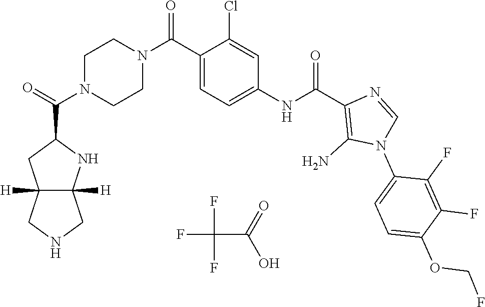 [SMILES: Nc1c(C(=O)Nc2ccc(C(=O)N3CCN(C(=O)[C@@H]4C[C@H]5CNC[C@H]5N4)CC3)c(Cl)c2)ncn1-c1ccc(OCF)c(F)c1F ; O=C(O)C(F)(F)F] | N-[4-[4-[(2S,3aS,6aS)-1,2,3,3a,4,5,6,6a- octahydropyrrolo[2,3-c]pyrrole-2- carbonyl]piperazine-1-carbonyl]-3- chlorophenyl]-5-amino-1-[2,3-difluoro-4- (fluoromethoxy)phenyl]imidazole-4- carboxamide. 1:2 2,2,2-trifluoroacetic acid |
| 355 | 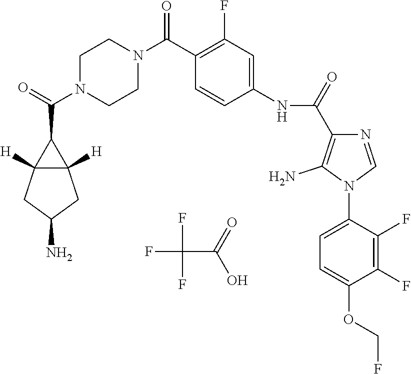 [SMILES: Nc1c(C(=O)Nc2ccc(C(=O)N3CCN(C(=O)[C@H]4[C@@H]5C[C@@H](N)C[C@@H]54)CC3)c(F)c2)ncn1-c1ccc(OCF)c(F)c1F ; O=C(O)C(F)(F)F] | 5-amino-N-[4-[4-[(1R,3s,5S,6r)-3- aminobicyclo[3.1.0]hexane-6- carbonyl]piperazine-1-carbonyl]-3- fluorophenyl]-1-[2,3-difluoro-4- (fluoromethoxy)phenyl]imidazole-4- carboxamide. 1:1 2,2,2-trifluoroacetic acid |
| 356 | 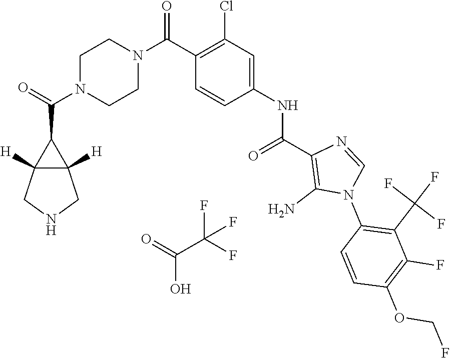 [SMILES: Nc1c(C(=O)Nc2ccc(C(=O)N3CCN(C(=O)[C@H]4[C@@H]5CNC[C@@H]54)CC3)c(Cl)c2)ncn1-c1ccc(OCF)c(F)c1C(F)(F)F ; O=C(O)C(F)(F)F] | 5-amino-N-[4-[4-[(1R,5S,6r)-3- azabicyclo[3.1.0]hexane-6- carbonyl]piperazine-1-carbonyl]-3- chlorophenyl]-1-[3-fluoro-4- (fluoromethoxy)-2- (trifluoromethyl)phenyl]imidazole-4- carboxamide. 1:1.5 2,2,2-trifluoroacetic acid |
| 357 | 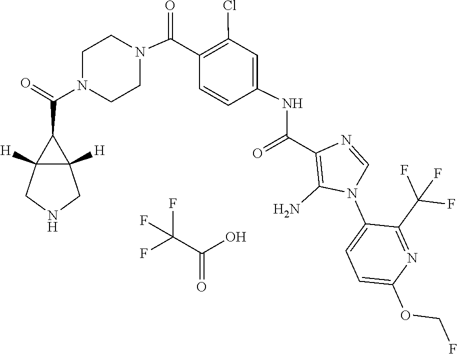 [SMILES: Nc1c(C(=O)Nc2ccc(C(=O)N3CCN(C(=O)[C@H]4[C@@H]5CNC[C@@H]54)CC3)c(Cl)c2)ncn1-c1ccc(OCF)nc1C(F)(F)F ; O=C(O)C(F)(F)F] | 5-amino-N-[4-[4-[(1-1R,5S,6r)-3- azabicyclo[3.1.0]hexane-6- carbonyl]piperazine-1-carbonyl]-3- chloro-phenyl]-1-[2-cyclopropyl-3-fluoro- 4-(fluoromethoxy)phenyl]imidazole-4- carboxamide; 2,2,2-trifluoroacetic acid |
| 358 | 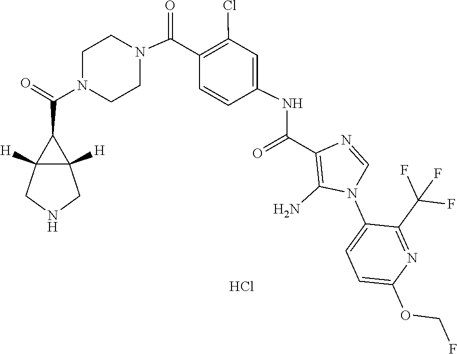 [SMILES: Nc1c(C(=O)Nc2ccc(C(=O)N3CCN(C(=O)[C@H]4[C@@H]5CNC[C@@H]54)CC3)c(Cl)c2)ncn1-c1ccc(OCF)nc1C(F)(F)F ; Cl] | 5-amino-N-[4-[4-[(1R,5S,6r)-3- azabicyclo[3.1.0]hexane-6- carbonyl]piperazine-1-carbonyl]-3- chlorophenyl]-1-[6-(fluoromethoxy)-2- (trifluoromethyl)-3-pyridyl]imidazole-4- carboxamide. 1:2 hydrogen chloride |
| 359 | 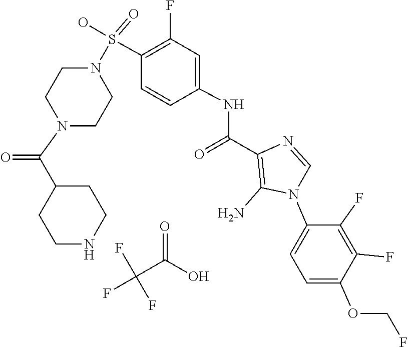 | 5-amino-1-[2,3-difluoro-4- (fluoromethoxy)phenyl]-N-[3-fluoro-4-(4- isonipecotoylpiperazino)sulphonylphenyl] imidazole-4-carboxamide. 1:1.5 2,2,2- trifluoroacetic acid |
| 360 | 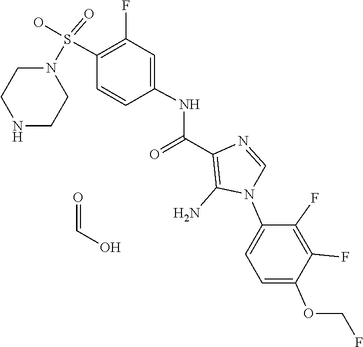 | 5-amino-1-[2,3-difluoro-4- (fluoromethoxy)phenyl]-N-(3-fluoro-4- piperazinosulphonylphenyl)imidazole-4- carboxamide. 1:0.23 formic acid |
| 361 | 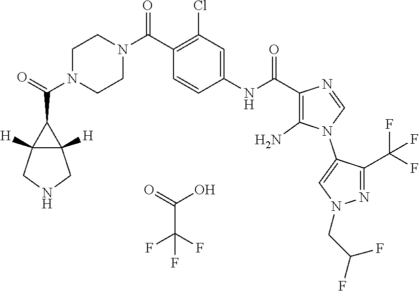 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC(F)F)nc1C(F)(F)F ; O=C(O)C(F)(F)F] | 5-amino-N-[4-[4-[(1R,5S,6r)-3- azabicyclo[3.1.0]hexane-6- carbonyl]piperazine-1-carbonyl]-3- chloro-phenyl]-1-[1-(2,2-difluoroethyl)-3- (trifluoromethyl)pyrazol-4-yl]imidazole-4- carboxamide. 1:1 2,2,2-trifluoroacetic acid |
| 362 | 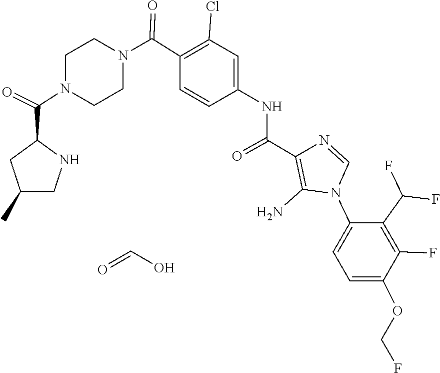 [SMILES: C[C@@H]1CN[C@H](C(=O)N2CCN(C(=O)c3ccc(NC(=O)c4ncn(-c5ccc(OCF)c(F)c5C(F)F)c4N)cc3Cl)CC2)C1 ; O=CO] | 5-amino-N-[3-chloro-4-[4-[(2S,4S)-4- methylpyrrolidine-2-carbonyl]piperazine- 1-carbonyl]phenyl]-1-[2-(difluoromethyl)- 3-fluoro-4- (fluoromethoxy)phenyl]imidazole-4- carboxamide; formic acid |
| 363 | 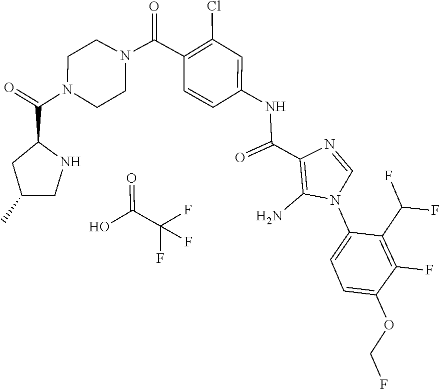 [SMILES: C[C@H]1CN[C@H](C(=O)N2CCN(C(=O)c3ccc(NC(=O)c4ncn(-c5ccc(OCF)c(F)c5C(F)F)c4N)cc3Cl)CC2)C1 ; O=C(O)C(F)(F)F] | 5-amino-N-[3-chloro-4-[4-[(2S,4R)-4- methylpyrrolidine-2-carbonyl]piperazine- 1-carbonyl]phenyl]-1-[2-(difluoromethyl)- 3-fluoro-4- (fluoromethoxy)phenyl]imidazole-4- carboxamide; 2,2,2-trifluoroacetic acid |
| 364 | 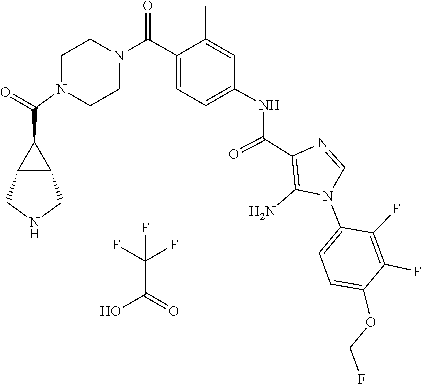 [SMILES: Cc1cc(NC(=O)c2ncn(-c3ccc(OCF)c(F)c3F)c2N)ccc1C(=O)N1CCN(C(=O)[C@H]2[C@@H]3CNC[C@@H]32)CC1 ; O=C(O)C(F)(F)F] | 5-Amino-N-[4-[4-[(1S,5R,6r)-3- azabicyclo[3.1.0]hexane-6- carbonyl]piperazine-1-carbonyl]-3- methylphenyl]-1-[2,3-difluoro-4- (fluoromethoxy)phenyl]imidazole-4- carboxamide; 2,2,2-trifluoroacetic acid |
| 365 | 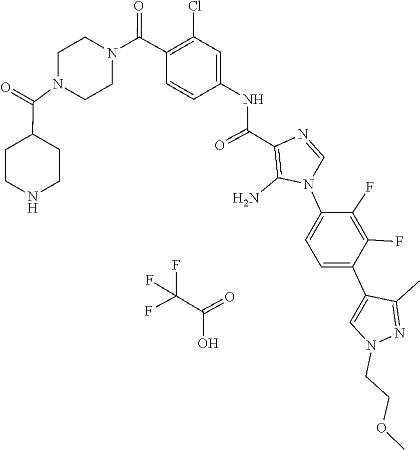 [SMILES: COCCn1cc(-c2ccc(-n3cnc(C(=O)Nc4ccc(C(=O)N5CCN(C(=O)C6CCNCC6)CC5)c(Cl)c4)c3N)c(F)c2F)c(C)n1 ; O=C(O)C(F)(F)F] | 5-Amino-N-[3-chloro-4-[4-(piperidine-4- carbonyl)piperazine-1-carbonyl]phenyl]- 1-[2,3-difluoro-4-[1-(2-methoxyethyl)-3- methyl-pyrazol-4-yl]phenyl]imidazole-4- carboxamide; 2,2,2-trifluoroacetic acid |
| 366 | 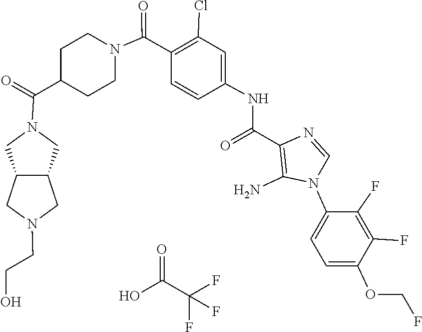 [SMILES: Nc1c(C(=O)Nc2ccc(C(=O)N3CCC(C(=O)N4C[C@H]5CN(CCO)C[C@H]5C4)CC3)c(Cl)c2)ncn1-c1ccc(OCF)c(F)c1F ; O=C(O)C(F)(F)F] | N-[4-[4-[(3aS,6aR)-2-(2-hydroxyethyl)- 1,3,3a,4,6,6a-hexahydropyrrolo[3,4- c]pyrrole-5-carbonyl]piperidine-1- carbonyl]-3-chloro-phenyl]-5-amino-1- [2,3-difluoro-4- (fluoromethoxy)phenyl]imidazole-4- carboxamide; 2,2,2-trifluoroacetic acid |
| 367 | 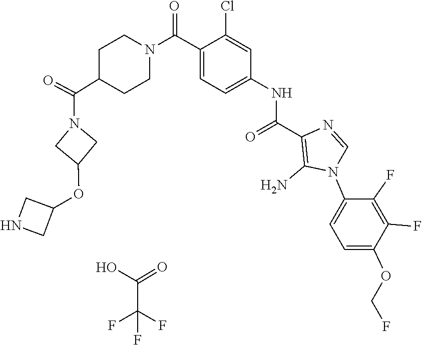 [SMILES: Nc1c(C(=O)Nc2ccc(C(=O)N3CCC(C(=O)N4CC(OC5CNC5)C4)CC3)c(Cl)c2)ncn1-c1ccc(OCF)c(F)c1F ; O=C(O)C(F)(F)F] | 5-Amino-N-[4-[4-[3-(azetidin-3- yloxy)azetidine-1-carbonyl]piperidine-1- carbonyl]-3-chlorophenyl]-1-[2,3- difluoro-4- (fluoromethoxy)phenyl]imidazole-4- carboxamide; 2,2,2-trifluoroacetic acid |
| 368 | 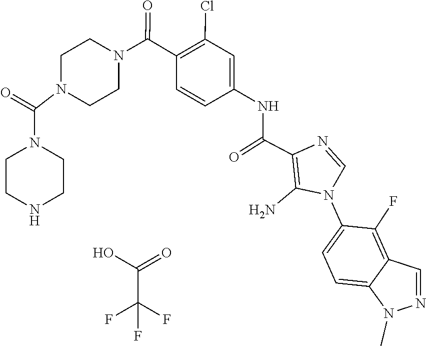 [SMILES: Cn1ncc2c(F)c(-n3cnc(C(=O)Nc4ccc(C(=O)N5CCN(C(=O)N6CCNCC6)CC5)c(Cl)c4)c3N)ccc21 ; O=C(O)C(F)(F)F] | 5-Amino-N-[3-chloro-4-[4-(piperazine-1- carbonyl)piperazine-1-carbonyl]phenyl]- 1-(4-fluoro-1-methyl-indazol-5- yl)imidazole-4-carboxamide; 2,2,2- trifluoroacetic acid |
| 369 | 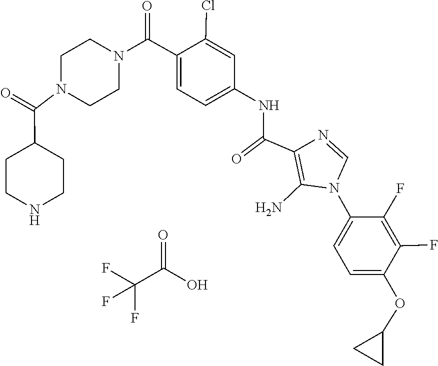 [SMILES: Nc1c(C(=O)Nc2ccc(C(=O)N3CCN(C(=O)C4CCNCC4)CC3)c(Cl)c2)ncn1-c1ccc(OC2CC2)c(F)c1F ; O=C(O)C(F)(F)F] | 5-amino-N-[3-chloro-4-[4-(piperidine-4- carbonyl)piperazine-1-carbonyl]phenyl]- 1-[4-(cyclopropoxy)-2,3-difluoro- phenyl]imidazole-4-carboxamide; 2,2,2- trifluoroacetic acid |
| 370 | 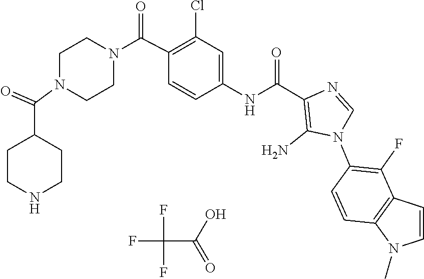 [SMILES: Cn1ccc2c(F)c(-n3cnc(C(=O)Nc4ccc(C(=O)N5CCN(C(=O)C6CCNCC6)CC5)c(Cl)c4)c3N)ccc21 ; O=C(O)C(F)(F)F] | 5-amino-N-[3-chloro-4-[4-(piperidine-4- carbonyl)piperazine-1-carbonyl]phenyl]- 1-(4-fluoro-1-methyl-indol-5- yl)imidazole-4-carboxamide; 2,2,2- trifluoroacetic acid |
| 371 | 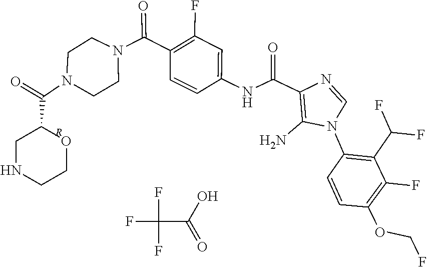 | 5-Amino-1-[2-(difluoromethyl)-3-fluoro- 4-(fluoromethoxy)phenyl]-N-[3-fluoro-4- [4-[(2R)-morpholine-2- carbonyl]piperazine-1- carbonyl]phenyl]imidazole-4- carboxamide; 2,2,2-trifluoroacetic acid |
| 372 | 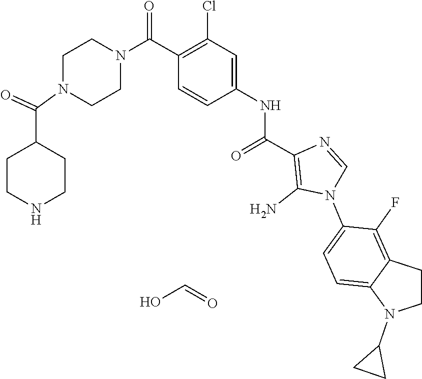 [SMILES: Nc1c(C(=O)Nc2ccc(C(=O)N3CCN(C(=O)C4CCNCC4)CC3)c(Cl)c2)ncn1-c1ccc2c(c1F)CCN2C1CC1 ; O=CO] | 5-Amino-N-[3-chloro-4-[4-(piperidine-4- carbonyl)piperazine-1-carbonyl]phenyl]- 1-(1-cyclopropyl-4-fluoroindolin-5- yl)imidazole-4-carboxamide; formic acid |
| 373 | 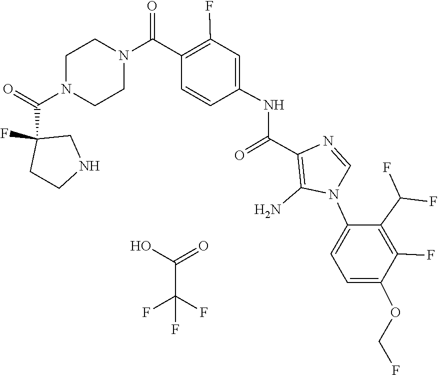 [SMILES: Nc1c(C(=O)Nc2ccc(C(=O)N3CCN(C(=O)[C@@]4(F)CCNC4)CC3)c(F)c2)ncn1-c1ccc(OCF)c(F)c1C(F)F ; O=C(O)C(F)(F)F] | 5-amino-1-[2-(difluoromethyl)-3-fluoro-4- (fluoromethoxy)phenyl]-N-[3-fluoro-4-[4- [(3R)-3-fluoropyrrolidine-3- carbonyl]piperazine-1- carbonyl]phenyl]imidazole-4- carboxamide; 2,2,2-trifluoroacetic acid |
| 374 | 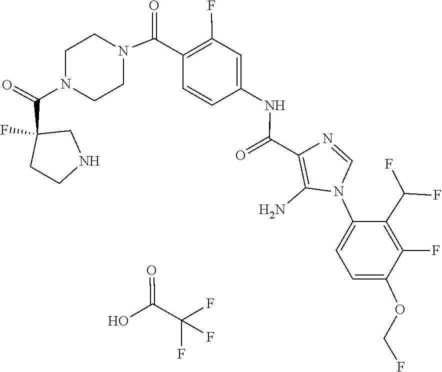 [SMILES: Nc1c(C(=O)Nc2ccc(C(=O)N3CCN(C(=O)[C@]4(F)CCNC4)CC3)c(F)c2)ncn1-c1ccc(OCF)c(F)c1C(F)F ; O=C(O)C(F)(F)F] | 5-amino-1-[2-(difluoromethyl)-3-fluoro-4- (fluoromethoxy)phenyl]-N-[3-fluoro-4-[4- [(3S)-3-fluoropyrrolidine-3- carbonyl]piperazine-1- carbonyl]phenyl]imidazole-4- carboxamide; 2,2,2-trifluoroacetic acid |
| 375 | 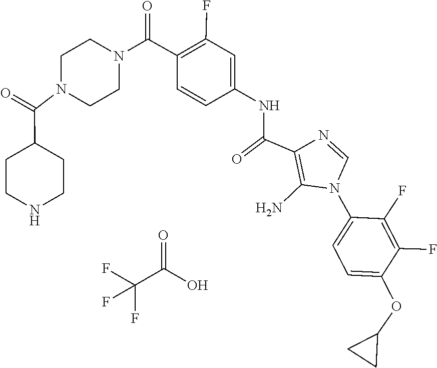 [SMILES: Nc1c(C(=O)Nc2ccc(C(=O)N3CCN(C(=O)C4CCNCC4)CC3)c(F)c2)ncn1-c1ccc(OC2CC2)c(F)c1F ; O=C(O)C(F)(F)F] | 5-Amino-1-[4-(cyclopropyloxy)-2,3- difluorophenyl]-N-[3-fluoro-4-[4- (piperazine-1-carbonyl)piperazine-1- carbonyl]phenyl]imidazole-4- carboxamide; 2,2,2-trifluoroacetic acid |
| 376 | 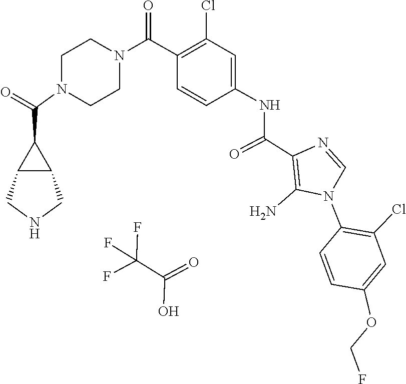 [SMILES: Nc1c(C(=O)Nc2ccc(C(=O)N3CCN(C(=O)[C@H]4[C@@H]5CNC[C@@H]54)CC3)c(Cl)c2)ncn1-c1ccc(OCF)cc1Cl ; O=C(O)C(F)(F)F] | 5-Amino-N-[4-[4-[(1R,5S,6r)-3- azabicyclo[3.1.0]hexane-6- carbonyl]piperazine-1-carbonyl]-3- chlorophenyl]-1-[2-chloro-4- (fluoromethoxy)phenyl]imidazole-4- carboxamide; 2,2,2-trifluoroacetic acid |
| 377 | 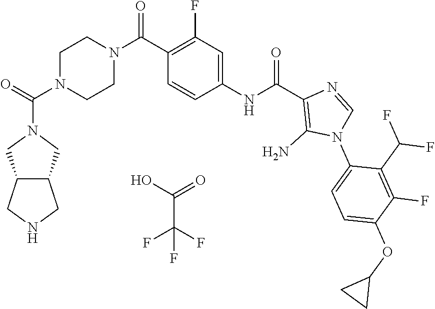 [SMILES: Nc1c(C(=O)Nc2ccc(C(=O)N3CCN(C(=O)N4C[C@H]5CNC[C@H]5C4)CC3)c(F)c2)ncn1-c1ccc(OC2CC2)c(F)c1C(F)F ; O=C(O)C(F)(F)F] | 5-Amino-1-[4-(cyclopropyloxy)-2- (difluoromethyl)-3-fluorophenyl]-N-[3- fluoro-4-[4-[(3aS,6aR)-2,3,3a,4,6,6a- hexahydro-1H-pyrrolo[3,4-c]pyrrole-5- carbonyl]piperazine-1- carbonyl]phenyl]imidazole-4- carboxamide; 2,2,2-trifluoroacetic acid |
| 378 | 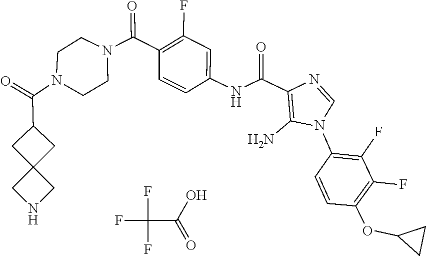 [SMILES: Nc1c(C(=O)Nc2ccc(C(=O)N3CCN(C(=O)C4CC5(CNC5)C4)CC3)c(F)c2)ncn1-c1ccc(OC2CC2)c(F)c1F ; O=C(O)C(F)(F)F] | 5-Amino-1-[4-(cyclopropyloxy)-2,3- difluorophenyl]-N-[4-[4-(2,6- diazaspiro[3.3]heptane-2- carbonyl)piperazine-1-carbonyl]-3- fluorophenyl]imidazole-4- carboxamide; 2,2,2-trifluoroacetic acid |
| 379 | 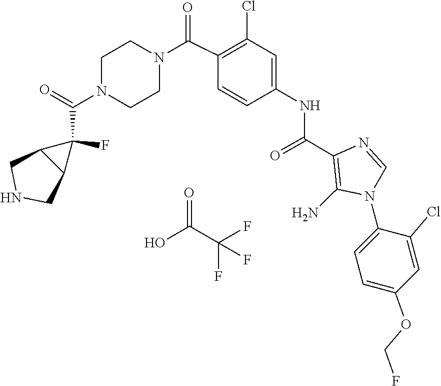 [SMILES: Nc1c(C(=O)Nc2ccc(C(=O)N3CCN(C(=O)[C@@]4(F)[C@@H]5CNC[C@@H]54)CC3)c(Cl)c2)ncn1-c1ccc(OCF)cc1Cl ; O=C(O)C(F)(F)F] | 5-Amino-N-[3-chloro-4-[4-[(1S,5R,6r)-6- fluoro-3-azabicyclo[3.1.0]hexane-6- carbonyl]piperazine-1-carbonyl]phenyl]- 1-[2-chloro-4- (fluoromethoxy)phenyl]imidazole-4- carboxamide; 2,2,2-trifluoroacetic acid |
| 380 | 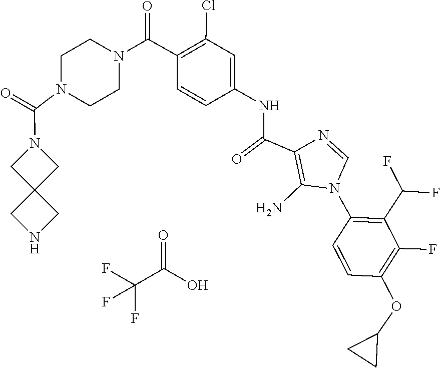 [SMILES: Nc1c(C(=O)Nc2ccc(C(=O)N3CCN(C(=O)N4CC5(CNC5)C4)CC3)c(Cl)c2)ncn1-c1ccc(OC2CC2)c(F)c1C(F)F ; O=C(O)C(F)(F)F] | 5-Amino-N-[3-chloro-4-[4-(2,6- diazaspiro[3.3]heptane-2- carbonyl)piperazine-1-carbonyl]phenyl]- 1-[4-(cyclopropyloxy)-2-(difluoromethyl)- 3-fluorophenyl]imidazole-4- carboxamide; 2,2,2-trifluoroacetic acid |
| 381 | 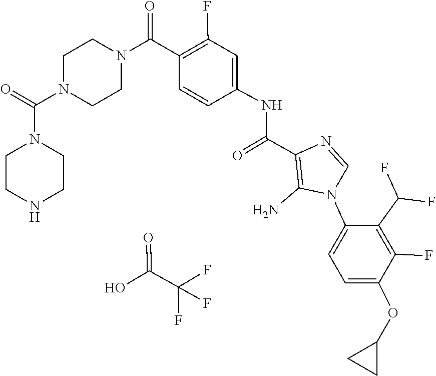 [SMILES: Nc1c(C(=O)Nc2ccc(C(=O)N3CCN(C(=O)N4CCNCC4)CC3)c(F)c2)ncn1-c1ccc(OC2CC2)c(F)c1C(F)F ; O=C(O)C(F)(F)F] | 5-Amino-1-[4-(cyclopropyloxy)-2- (difluoromethyl)-3-fluorophenyl]-N-[3- fluoro-4-[4-(piperazine-1- carbonyl)piperazine-1- carbonyl]phenyl]imidazole-4- carboxamide; 2,2,2-trifluoroacetic acid |
| 382 | 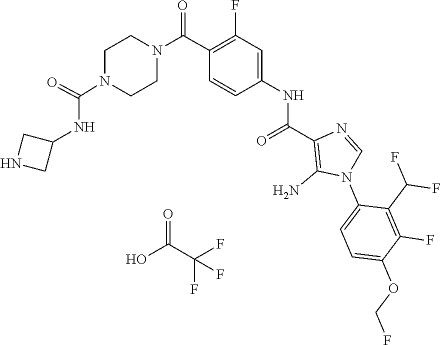 [SMILES: Nc1c(C(=O)Nc2ccc(C(=O)N3CCN(C(=O)NC4CNC4)CC3)c(F)c2)ncn1-c1ccc(OCF)c(F)c1C(F)F ; O=C(O)C(F)(F)F] | 4-[4-[[5-Amino-1-[2-(difluoromethyl)-3- fluoro-4- (fluoromethoxy)phenyl]imidazole-4- carbonyl]amino]-2-fluorobenzoyl]-N- (azetidin-3-yl)piperazine-1- carboxamide; 2,2,2-trifluoroacetic acid |
| 383 | 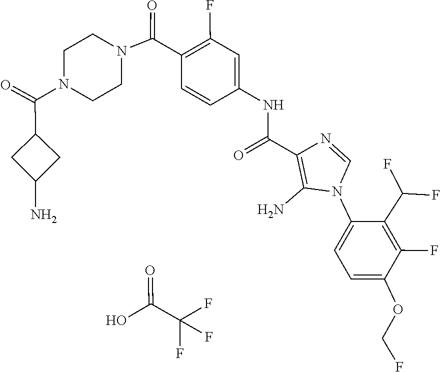 [SMILES: Nc1c(C(=O)Nc2ccc(C(=O)N3CCN(C(=O)C4CC(N)C4)CC3)c(F)c2)ncn1-c1ccc(OCF)c(F)c1C(F)F ; O=C(O)C(F)(F)F] | 5-Amino-N-[4-[4-(3-aminoazetidine-1- carbonyl)piperazine-1-carbonyl]-3- fluorophenyl]-1-[2-(difluoromethyl)-3- fluoro-4- (fluoromethoxy)phenyl]imidazole-4- carboxamide; 2,2,2-trifluoroacetic acid |
| 384 | 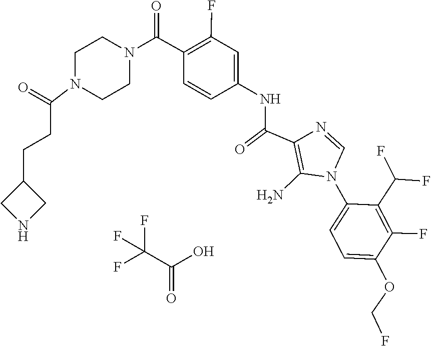 [SMILES: Nc1c(C(=O)Nc2ccc(C(=O)N3CCN(C(=O)CCC4CNC4)CC3)c(F)c2)ncn1-c1ccc(OCF)c(F)c1C(F)F ; O=C(O)C(F)(F)F] | 4-[4-[[5-Amino-1-[2-(difluoromethyl)-3- fluoro-4- (fluoromethoxy)phenyl]imidazole-4- carbonyl]amino]-2-fluorobenzoyl]-N- (azetidin-3-ylmethyl)piperazine-1- carboxamide; 2,2,2-trifluoroacetic acid |
| 385 | 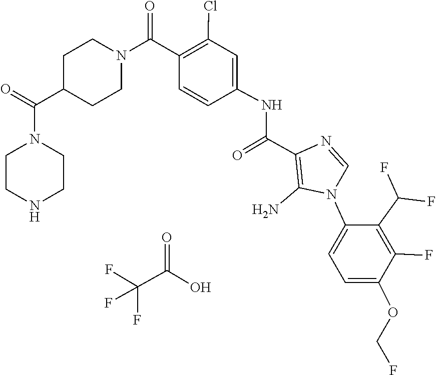 [SMILES: Nc1c(C(=O)Nc2ccc(C(=O)N3CCC(C(=O)N4CCNCC4)CC3)c(Cl)c2)ncn1-c1ccc(OCF)c(F)c1C(F)F ; O=C(O)C(F)(F)F] | 5-Amino-N-[3-chloro-4-[4-(piperazine-1- carbonyl)piperidine-1-carbonyl]phenyl]- 1-[2-(difluoromethyl)-3-fluoro-4- (fluoromethoxy)phenyl]imidazole-4- carboxamide; 2,2,2-trifluoroacetic acid |
| 386 | 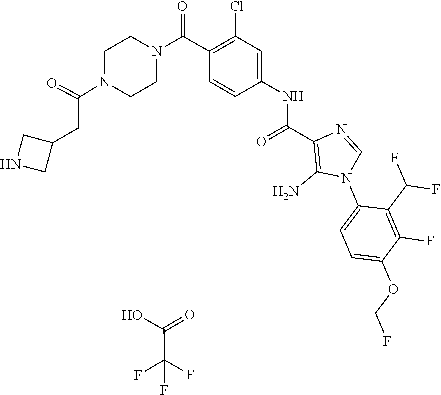 [SMILES: Nc1c(C(=O)Nc2ccc(C(=O)N3CCN(C(=O)CC4CNC4)CC3)c(Cl)c2)ncn1-c1ccc(OCF)c(F)c1C(F)F ; O=C(O)C(F)(F)F] | 5-Amino-N-[4-[4-[2-(azetidin-3- yl) acetyl]piperazine-1-carbonyl]-3- chlorophenyl]-1-[2-(difluoromethyl)-3- fluoro-4- (fluoromethoxy)phenyl]imidazole-4- carboxamide; 2,2,2-trifluoroacetic acid |
| 387 | 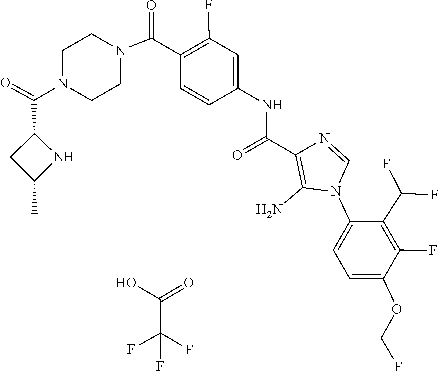 [SMILES: C[C@@H]1C[C@H](C(=O)N2CCN(C(=O)c3ccc(NC(=O)c4ncn(-c5ccc(OCF)c(F)c5C(F)F)c4N)cc3F)CC2)N1 ; O=C(O)C(F)(F)F] | 5-Amino-1-[2-(difluoromethyl)-3-fluoro- 4-(fluoromethoxy)phenyl]-N-[3-fluoro-4- [4-[(2R,4R)-4-methylazetidine-2- carbonyl]piperazine-1- carbonyl]phenyl]imidazole-4- carboxamide; 2,2,2-trifluoroacetic acid |
| 388 | 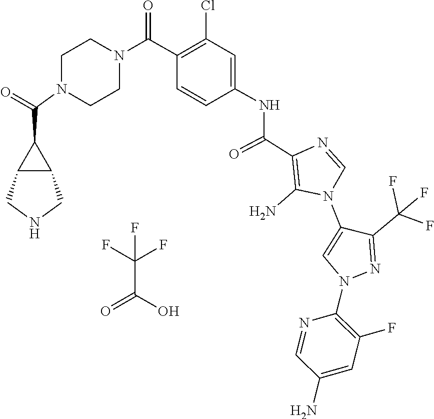 [SMILES: Nc1cnc(-n2cc(-n3cnc(C(=O)Nc4ccc(C(=O)N5CCN(C(=O)[C@H]6[C@@H]7CNC[C@@H]76)CC5)c(Cl)c4)c3N)c(C(F)(F)F)n2)c(F)c1 ; O=C(O)C(F)(F)F] | 5-Amino-1-[1-(5-amino-3-fluoro-2- pyridyl)-3-(trifluoromethyl)pyrazol-4-yl]- N-[3-chloro-4-[4-[(1S,5R,6r)-3- azabicyclo[3.1.0]hexane-6- carbonyl]piperazine-1- carbonyl]phenyl]imidazole-4- carboxamide; 2,2,2-trifluoroacetic acid |
| 389 | 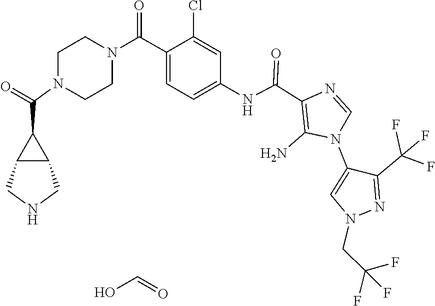 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC(F)(F)F)nc1C(F)(F)F ; O=CO] | 5-Amino-N-[3-chloro-4-[4-[(1S,5R,6r)-3- azoniabicyclo[3.1.0]hexane-6- carbonyl]piperazine-1-carbonyl]phenyl]- 1-[1-(2,2,2-trifluoroethyl)-3- (trifluoromethyl)pyrazol-4-yl]imidazole-4- carboxamide; formate |
| 390 | 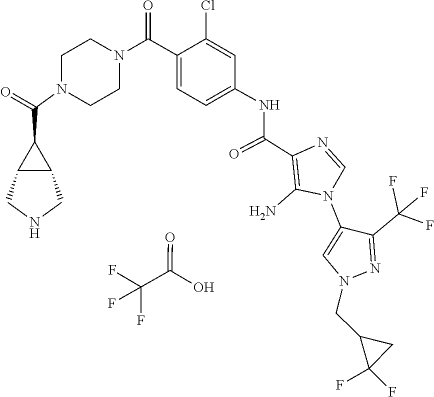 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2CC2(F)F)nc1C(F)(F)F ; O=C(O)C(F)(F)F] | 5-Amino-N-[3-chloro-4-[4-[(1S,5R,6r)-3- azabicyclo[3.1.0]hexane-6- carbonyl]piperazine-1-carbonyl]phenyl]- 1-[1-[(2,2-difluorocyclopropyl)methyl]-3- (trifluoromethyl)pyrazol-4-yl]imidazole-4- carboxamide; 2,2,2-trifluoroacetic acid |
| 391 | 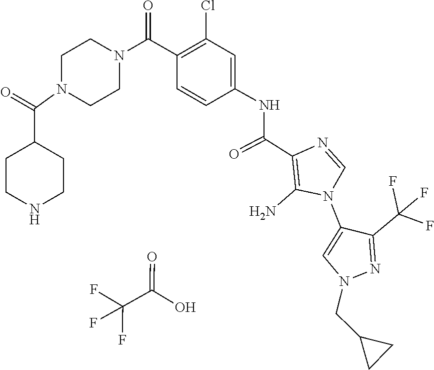 [SMILES: Nc1c(C(=O)Nc2ccc(C(=O)N3CCN(C(=O)C4CCNCC4)CC3)c(Cl)c2)ncn1-c1cn(CC2CC2)nc1C(F)(F)F ; O=C(O)C(F)(F)F] | 5-Amino-N-[3-chloro-4-[4-(piperidine-4- carbonyl)piperazine-1-carbonyl]phenyl]- 1-[1-(cyclopropylmethyl)-3- (trifluoromethyl)pyrazol-4-yl]imidazole-4- carboxamide; 2,2,2-trifluoroacetic acid |
| 392 | 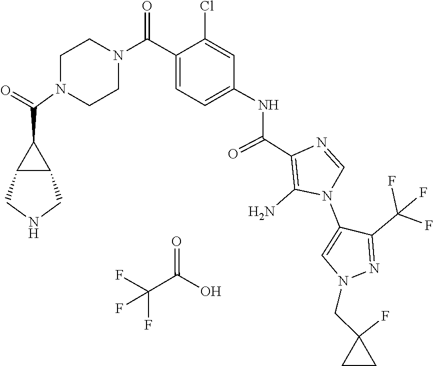 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2(F)CC2)nc1C(F)(F)F ; O=C(O)C(F)(F)F] | 5-Amino-N-[4-[4-[(1S,5R,6r)-3- azabicyclo[3.1.0]hexane-6- carbonyl]piperazine-1-carbonyl]-3- chlorophenyl]-1-[1-[(1- fluorocyclopropyl)methyl]-3- (trifluoromethyl)pyrazol-4-yl]imidazole-4- carboxamide |
| 393 | 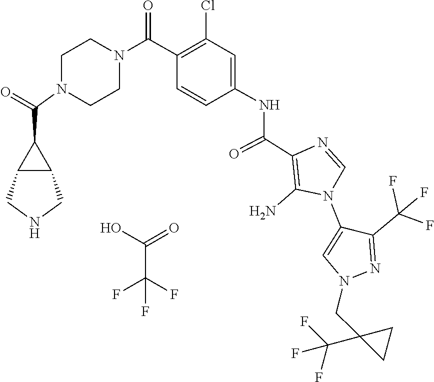 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2(C(F)(F)F)CC2)nc1C(F)(F)F ; O=C(O)C(F)(F)F] | 5-Amino-N-[3-chloro-4-[4-[(1S,5R,6r)-3- azabicyclo[3.1.0]hexane-6- carbonyl]piperazine-1-carbonyl]phenyl]- 1-[3-(trifluoromethyl)-1-[1- (trifluoromethyl) cyclopropyl]methyl] pyrazol-4-yl]imidazole- 4-carboxamide; 2,2,2- trifluoroacetic acid |
| 394 | 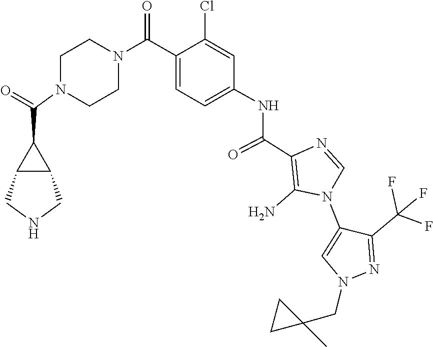 [SMILES: CC1(Cn2cc(-n3cnc(C(=O)Nc4ccc(C(=O)N5CCN(C(=O)[C@H]6[C@@H]7CNC[C@@H]76)CC5)c(Cl)c4)c3N)c(C(F)(F)F)n2)CC1] | 5-Amino-N-[3-chloro-4-[4-[(1S,5R,6r)-3- azabicyclo[3.1.0]hexane-6- carbonyl]piperazine-1-carbonyl]phenyl]- 1-[1-[(1-methylcyclopropyl)methyl]-3- (trifluoromethyl)pyrazol-4-yl]imidazole-4- carboxamide |
| 395 | 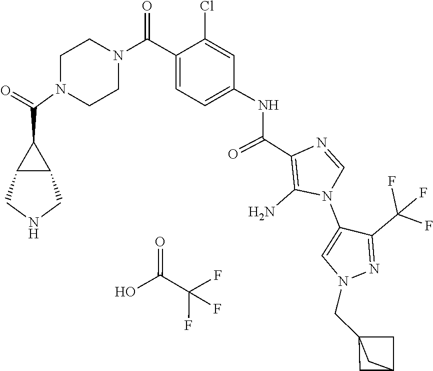 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC23CC(C2)C3)nc1C(F)(F)F ; O=C(O)C(F)(F)F] | 5-Amino-N-[4-[4-[(1S,5R,6r)-3- azabicyclo[3.1.0]hexane-6- carbonyl]piperazine-1-carbonyl]-3- chlorophenyl]-1-[1-(1- bicyclo[1.1.1]pentanylmethyl)-3- (trifluoromethyl)pyrazol-4-yl]imidazole-4- carboxamide; 2,2,2-trifluoroacetic acid |
| 396 | 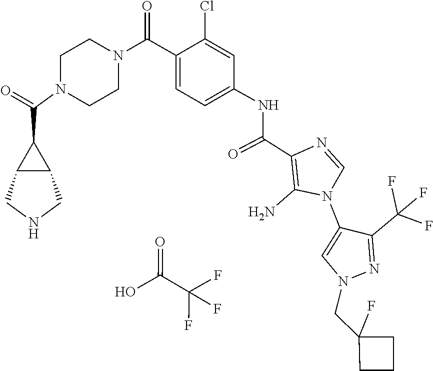 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2(F)CCC2)nc1C(F)(F)F ; O=C(O)C(F)(F)F] | 5-Amino-N-[4-[4-[(1S,5R,6r)-3- azabicyclo[3.1.0]hexane-6- carbonyl]piperazine-1-carbonyl]-3- chlorophenyl]-1-[1-[(1- fluorocyclobutyl)methyl]-3- (trifluoromethyl)pyrazol-4-yl]imidazole-4- carboxamide; 2,2,2-trifluoroacetic acid |
| 397 | 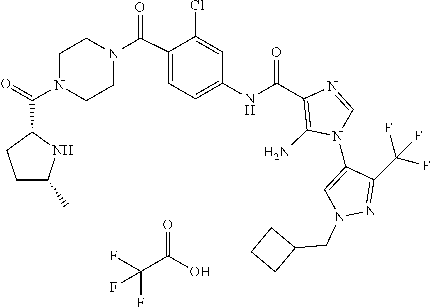 [SMILES: C[C@@H]1CC[C@H](C(=O)N2CCN(C(=O)c3ccc(NC(=O)c4ncn(-c5cn(CC6CCC6)nc5C(F)(F)F)c4N)cc3Cl)CC2)N1 ; O=C(O)C(F)(F)F] | 5-Amino-N-[3-chloro-4-[4-[(2R,5R)-5- methylpyrrolidine-2-carbonyl]piperazine- 1-carbonyl]phenyl]-1-[1- (cyclobutylmethyl)-3- (trifluoromethyl)pyrazol-4-yl]imidazole-4- carboxamide; 2,2,2-trifluoroacetic acid |
| 398 | 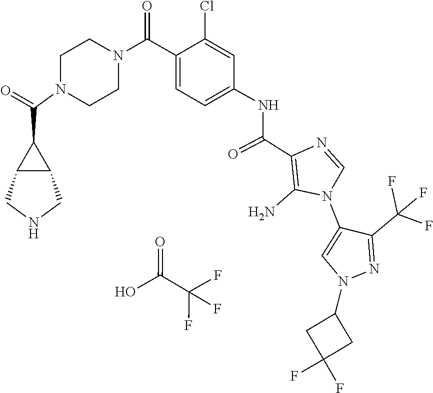 [SMILES: Nc1c(C(=O)Nc2ccc(C(=O)N3CCN(C(=O)[C@H]4[C@@H]5CNC[C@@H]54)CC3)c(Cl)c2)ncn1-c1cn(C2CC(F)(F)C2)nc1C(F)(F)F ; O=C(O)C(F)(F)F] | 5-Amino-N-[3-chloro-4-[4-[(1R,5S,6r)-3- azabicyclo[3.1.0]hexane-6- carbonyl]piperazine-1-carbonyl]phenyl]- 1-[1-(3,3-difluorocyclobutyl)-3- (trifluoromethyl)pyrazol-4-yl]imidazole-4- carboxamide; 2,2,2-trifluoroacetic acid |
| 399 | 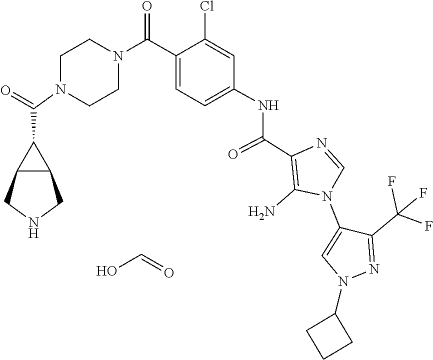 [SMILES: Nc1c(C(=O)Nc2ccc(C(=O)N3CCN(C(=O)[C@H]4[C@@H]5CNC[C@@H]54)CC3)c(Cl)c2)ncn1-c1cn(C2CCC2)nc1C(F)(F)F ; O=CO] | 5-Amino-N-[4-[4-[(1R,5S,6r)-3- azabicyclo[3.1.0]hexane-6- carbonyl]piperazine-1-carbonyl]-3- chlorophenyl]-1-[1-cyclobutyl-3- (trifluoromethyl)pyrazol-4-yl]imidazole-4- carboxamide; formic acid |
| 400 | 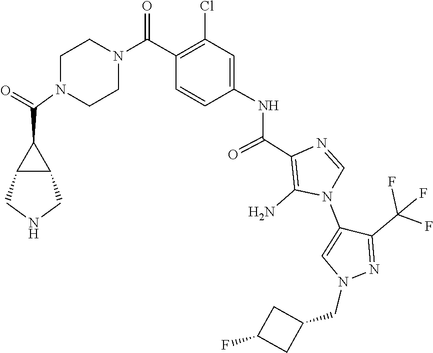 [SMILES: Nc1c(C(=O)Nc2ccc(C(=O)N3CCN(C(=O)[C@H]4[C@@H]5CNC[C@@H]54)CC3)c(Cl)c2)ncn1-c1cn(C[C@H]2C[C@@H](F)C2)nc1C(F)(F)F] | 5-amino-N-[4-[4-[(1S,5R,6r)-3- azabicyclo[3.1.0]hexane-6- carbonyl]piperazine-1-carbonyl]-3- chloro-phenyl]-1-[1-[cis((3- fluorocyclobutyl)methyl)]-3- (trifluoromethyl)pyrazol-4-yl]imidazole-4- carboxamide |
| 401 | 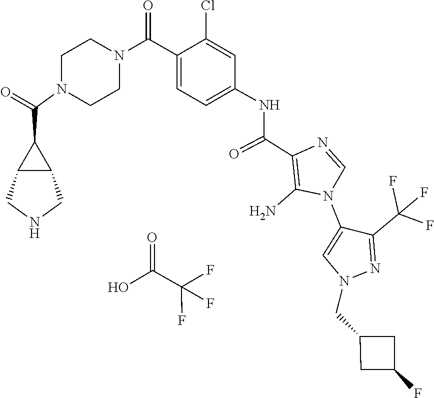 [SMILES: Nc1c(C(=O)Nc2ccc(C(=O)N3CCN(C(=O)[C@H]4[C@@H]5CNC[C@@H]54)CC3)c(Cl)c2)ncn1-c1cn(C[C@H]2C[C@H](F)C2)nc1C(F)(F)F ; O=C(O)C(F)(F)F] | N-(4-(4-((1R,5S,6r)-3- azabicyclo[3.1.0]hexane-6- carbonyl)piperazine-1-carbonyl)-3- chlorophenyl)-5-amino-1-(1-(((1r,3r)-3- fluorocyclobutyl)methyl)-3- (trifluoromethyl)-1H-pyrazol-4-yl)-1H- imidazole-4-carboxamide 2,2,2- trifluoroacetate |
| 402 | 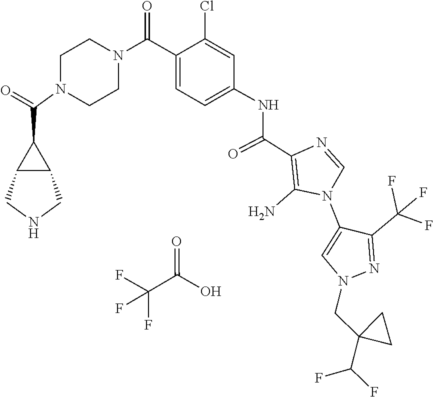 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2(C(F)F)CC2)nc1C(F)(F)F ; O=C(O)C(F)(F)F] | 5-Amino-N-[3-chloro-4-[4-[(1S,5R,6r)-3- azabicyclo[3.1.0]hexane-6- carbonyl]piperazine-1-carbonyl]phenyl]- 1-[1-[[1- (difluoromethyl)cyclopropyl]methyl]-3- (trifluoromethyl)pyrazol-4-yl]imidazole-4- carboxamide; 2,2,2-trifluoroacetic acid |
| 403 | 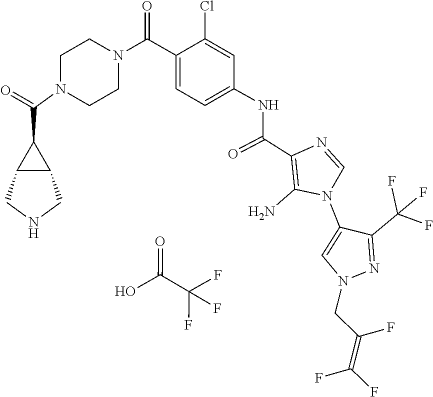 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC(F)=C(F)F)nc1C(F)(F)F ; O=C(O)C(F)(F)F] | 5-Amino-N-[4-[4-[(1S,5R,6r)-3- azabicyclo[3.1.0]hexane-6- carbonyl]piperazine-1-carbonyl]-3- chlorophenyl]-1-[1-(2,3,3-trifluoroallyl)-3- (trifluoromethyl)pyrazol-4-yl]imidazole-4- carboxamide; 2,2,2-trifluoroacetic acid |
| 404 | 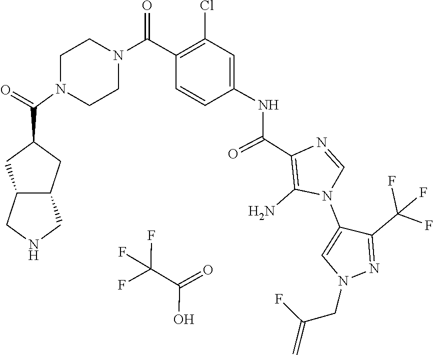 [SMILES: C=C(F)Cn1cc(-n2cnc(C(=O)Nc3ccc(C(=O)N4CCN(C(=O)[C@@H]5C[C@H]6CNC[C@H]6C5)CC4)c(Cl)c3)c2N)c(C(F)(F)F)n1 ; O=C(O)C(F)(F)F] | N-[4-[4-[(3aR,6aS)-1,2,3,3a,4,5,6,6a- Octahydrocyclopenta[c]pyrrole-5- carbonyl]piperazine-1-carbonyl]-3- chlorophenyl]-5-amino-1-[1-(2- fluoroallyl)-3-(trifluoromethyl)pyrazol-4- yl]imidazole-4-carboxamide; 2,2,2- trifluoroacetic acid |
| 405 | 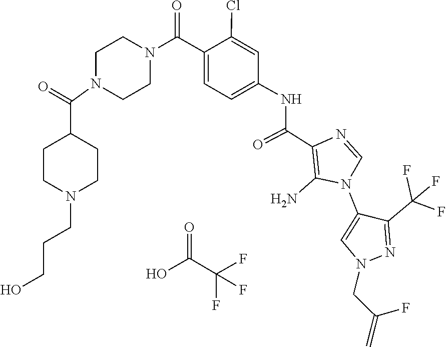 [SMILES: C=C(F)Cn1cc(-n2cnc(C(=O)Nc3ccc(C(=O)N4CCN(C(=O)C5CCN(CCCO)CC5)CC4)c(Cl)c3)c2N)c(C(F)(F)F)n1 ; O=C(O)C(F)(F)F] | 5-Amino-N-[3-chloro-4-[4-[1-(3- hydroxypropyl)piperidine-4- carbonyl]piperazine-1-carbonyl]phenyl]- 1-[1-(2-fluoroallyl)-3- (trifluoromethyl)pyrazol-4-yl]imidazole-4- carboxamide; 2,2,2-trifluoroacetic acid |
| 406 | 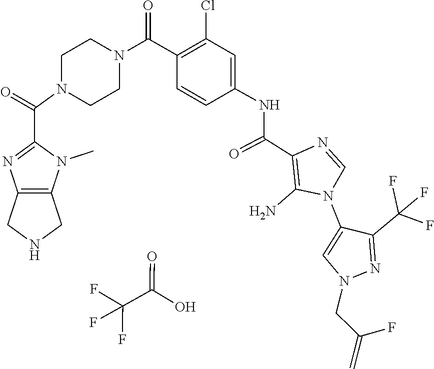 [SMILES: C=C(F)Cn1cc(-n2cnc(C(=O)Nc3ccc(C(=O)N4CCN(C(=O)c5nc6c(n5C)CNC6)CC4)c(Cl)c3)c2N)c(C(F)(F)F)n1 ; O=C(O)C(F)(F)F] | 5-Amino-N-[3-chloro-4-[4-(3-methyl-5,6- dihydro-4H-pyrrolo[3,4-d]imidazole-2- carbonyl)piperazine-1-carbonyl]phenyl]- 1-[1-(2-fluoroallyl)-3- (trifluoromethyl)pyrazol-4-yl]imidazole-4- carboxamide; 2,2,2-trifluoroacetic acid |
| 407 | 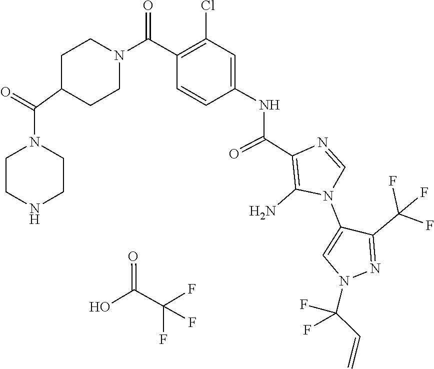 [SMILES: C=CC(F)(F)n1cc(-n2cnc(C(=O)Nc3ccc(C(=O)N4CCC(C(=O)N5CCNCC5)CC4)c(Cl)c3)c2N)c(C(F)(F)F)n1 ; O=C(O)C(F)(F)F] | 5-Amino-N-[3-chloro-4-[4-(piperazine-1- carbonyl)piperidine-1-carbonyl]phenyl]- 1-[1-(1,1-difluoroallyl)-3- (trifluoromethyl)pyrazol-4-yl]imidazole-4- carboxamide; 2,2,2-trifluoroacetic acid |
| 408 | 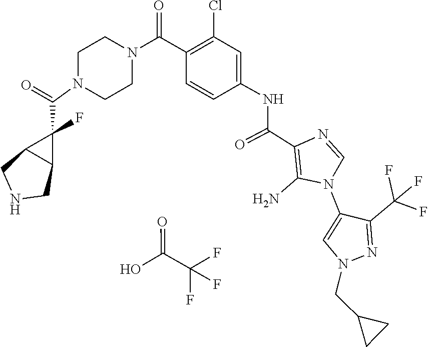 [SMILES: Nc1c(C(=O)Nc2ccc(C(=O)N3CCN(C(=O)[C@@]4(F)[C@@H]5CNC[C@@H]54)CC3)c(Cl)c2)ncn1-c1cn(CC2CC2)nc1C(F)(F)F ; O=C(O)C(F)(F)F] | 5-Amino-N-[3-chloro-4-[4-[(1S,5R,6r)-6- fluoro-3-azabicyclo[3.1.0]hexane-6- carbonyl]piperazine-1-carbonyl]phenyl]- 1-[1-(cyclopropylmethyl)-3- (trifluoromethyl)pyrazol-4-yl]imidazole-4- carboxamide; 2,2,2-trifluoroacetic acid |

### General Synthetic Methods

Compounds or a stereoisomer, tautomer, or pharmaceutically acceptable salt thereof as described herein of the present disclosure can be made by a variety of methods depicted in the synthetic reaction schemes shown and described herein. The starting materials and reagents used in preparing these compounds generally are either available from commercial suppliers or are prepared by methods known to those skilled in the art following procedures set forth in references such as Fieser and Fieser's Reagents for Organic Synthesis; Wiley & Sons: New York, vol. 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds.) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, vol. 1-40. T Synthetic chemistry transformations and protecting group methodologies (protection and deprotection) useful in synthesizing compounds described herein and necessary reagents and intermediates include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

The Examples provide exemplary methods for preparing compounds or a stereoisomer, tautomer, or pharmaceutically acceptable salt thereof as described herein. Those skilled in the art will appreciate that other synthetic routes can be used to synthesize the compounds or a stereoisomer, tautomer, or pharmaceutically acceptable salt thereof as described herein. Although specific starting materials and reagents are depicted and discussed in the Examples, other starting materials and reagents can be substituted to provide a variety of derivatives and/or reaction conditions. In addition, many of the exemplary compounds prepared by the described methods can be further modified in light of this disclosure using conventional chemistry.

In preparing compounds or a stereoisomer, tautomer, or pharmaceutically acceptable salt thereof as described herein protection of remote functionality (e.g., primary or secondary amine) of intermediates can be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz) and 9-fluorenylmethyleneoxycarbonyl (Fmoc). The need for such protection can be readily determined. For a general description of protecting groups and their use, see T. W. Greene, Protective Groups in Organic Synthesis, John Wiley & Sons, New York, 1991.

In the methods of preparing compounds or a stereoisomer, tautomer, or pharmaceutically acceptable salt thereof as described herein, it can be advantageous to separate reaction products from one another and/or from starting materials. The desired products of each step or series of steps may be separated and/or purified to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like. Selection of appropriate methods of separation depends on the nature of the materials involved, such as, boiling point and molecular weight in distillation and sublimation, presence or absence of polar functional groups in chromatography, stability of materials in acidic and basic media in multiphase extraction, and the like.

Diastereomeric mixtures can be separated into their individual diastereomers on the basis of their physical chemical differences by methods such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers.

A single stereoisomer, e.g., an enantiomer, substantially free of its stereoisomer can be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113 (3): 283-302). Racemic mixtures of chiral compounds or pharmaceutically acceptable salts thereof described herein can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

The chemical reactions described herein may be readily adapted to prepare other compounds and pharmaceutically acceptable salts thereof described herein. For example, the synthesis of non-exemplified compounds and pharmaceutically acceptable salts thereof described herein may be successfully performed by modifications apparent to those skilled in the art, e.g., by appropriately protecting interfering groups, by utilizing other suitable reagents known in the art other than those described, or by making routine modifications of reaction conditions. Alternatively, other reactions disclosed herein or known in the art will be recognized as having applicability for preparing other compounds and pharmaceutically acceptable salts thereof described herein.

The following exemplary abbreviations are used in the present text:
Å Angstrom (10⁻¹⁰ m)
ACN or Acetonitrile
MeCN
Aq. aqueous
BINAP 2,2′-bis(diphenylphosphino)-1,1′-binaphthyl
BOC or Boc tert-butyloxycarbonyl
CDI carbonyldiimidazole
CFU colony-forming unit
D day(s)
DCM dichloromethane
DEA Diethylamine
DIEA N,N-diisopropylethylamine
DIPEA N,N-diisopropylethylamine
DMF N,N-dimethylformamide
EDTA ethylenediaminetetraacetic acid
EtOAc or EA ethyl acetate
EtOH ethanol
Eq equivalent(s)
FA Formic acid
FMOC or fluorenylmethoxycarbonyl
Fmoc
h(s) or hr(s) hour
HATU: 1-[bis(dimethylamino)methylene]-1H-1,2,3-triazolo[4,5-b]pyridinium 3-oxid hexafluorophosphate
HPLC: high performance liquid chromatography
HPLC-UV: high performance liquid chromatography with ultraviolet detector
HV high vacuum
IC50 half maximal inhibitory concentration
IC90 90% inhibitory concentration
LED light-emitting diode
[M] molecular mass
M molar
MeOH Methanol
min minute(s)
mL milliliter(s)
MS mass spectrometry
NaBH₃CN Sodium cyanoborohydride
O/N overnight, approximately 16 hours
PE petroleum ether
PdCl₂(DPPF) [1,1′-bis(diphenylphosphino)ferrocene]dichloropalladium(II)
Pd₂(dba)₃ tris(dibenzylideneacetone)dipalladium(0)
PG protective group
Precat precatalyst
PyAOP (7-azabenzotriazol-1-yloxy)tripyrrolidinophosphonium hexafluorophosphate
quant quantitative
rac racemic
RP reverse phase
RPHPLC reverse-phase high-performance liquid chromatography
Rt retention time
RT room temperature, approximately 23° C.
sat saturated
SEM 2-methoxyethyl(trimethyl)silane
SFC supercritical fluid chromatography
T3P propylphosphonic anhydride solution
TEMPO (2,2,6,6-tetramethylpiperidin-1-yl)oxyl
TFA trifluoroacetic acid
THF tetrahydrofuran
wt weight
XANTPHOS 4,5-bis(diphenylphosphino)-9,9-dimethylxanthene

### Pharmaceutical Compositions

Also provided herein are pharmaceutical compositions comprising compound or a stereoisomer, tautomer, or pharmaceutically acceptable salt thereof as described herein and one or more pharmaceutically acceptable excipients.

Compounds or a stereoisomer, tautomer, or pharmaceutically acceptable salt thereof as described herein as described herein can be formulated in accordance with standard pharmaceutical practice as a pharmaceutical composition. Thus, further provided herein is a pharmaceutical composition comprising a compound or a stereoisomer, tautomer, or pharmaceutically acceptable salt thereof as described herein as described herein and one or more pharmaceutically acceptable excipients.

Suitable carriers, diluents and excipients include, but are not limited to, materials such as carbohydrates, waxes, water soluble and/or swellable polymers, hydrophilic or hydrophobic materials, gelatin, oils, solvents, water and the like. The particular excipient used will depend upon the means and purpose for which the compound or pharmaceutically acceptable salt thereof as described herein is being applied.

The pharmaceutical composition (or formulation) for application can be packaged in a variety of ways depending upon the method used for administering the drug. Generally, an article for distribution includes a container having deposited therein the pharmaceutical formulation in an appropriate form. Suitable containers include materials such as bottles (plastic and glass), sachets, ampoules, plastic bags, metal cylinders, and the like. The container can also include a tamper-proof assemblage to prevent indiscreet access to the contents of the package. In addition, the container has deposited thereon a label that describes the contents of the container. The label can also include appropriate warnings.

The pharmaceutical composition ordinarily can be stored as a solid composition, a lyophilized formulation or as an aqueous solution.

The pharmaceutical compositions described herein can be formulated, dosed and administered in a fashion, i.e., amounts, concentrations, schedules, course, vehicles and route of administration, consistent with good medical practice. Factors for consideration in this context include the particular disorder being treated, the particular patient being treated, the clinical condition of the individual patient, the cause of the disorder, the site of delivery of the agent, the method of administration, the scheduling of administration, and other factors known to medical practitioners. The effective amount of the compound or a stereoisomer, tautomer, or pharmaceutically acceptable salt thereof to be administered will be governed by such considerations, and is the minimum amount necessary to ameliorate, or treat the hyperproliferative disorder.

The formulations include those suitable for the administration routes detailed herein. The formulations can conveniently be presented in unit dosage form and can be prepared by any methods. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound or a stereoisomer, tautomer, or pharmaceutically acceptable salt thereof as described herein suitable for oral administration can be prepared as discrete units such as pills, capsules, cachets or tablets each containing a predetermined amount of such compound or a stereoisomer, tautomer, or pharmaceutically acceptable salt thereof. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom. Tablets, troches, lozenges, aqueous or oil suspensions, dispersible powders or granules, emulsions, hard or soft capsules, e.g., gelatin capsules, syrups or elixirs can be prepared for oral use. Formulations of compounds or pharmaceutically acceptable salts thereof as described herein intended for oral use can be prepared according to any method for the manufacture of pharmaceutical compositions and such compositions can contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

The pharmaceutical compositions of a compound or a stereoisomer, tautomer, or pharmaceutically acceptable salt thereof as described herein can be in the form of a sterile injectable preparation, such as a sterile injectable aqueous or oleaginous suspension. This suspension can be formulated using suitable dispersing or wetting agents and suspending agents which have been mentioned above. The sterile injectable preparation can also be a sterile injectable solution or suspension in a non-toxic parenterally acceptable diluent or solvent, such as a solution in 1,3-butanediol or prepared as a lyophilized powder. Among the acceptable vehicles and solvents that can be employed are water, Ringer's solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

In one embodiment, the compound or a stereoisomer, tautomer, or pharmaceutically acceptable salt thereof are formulated as a prodrug. The term prodrug as used herein refers to a derivative of a compound that can be hydrolyzed, oxidized, or cleaved under biological conditions to provide the compound or a stereoisomer, tautomer, or pharmaceutically acceptable salt thereof. A prodrug as defined herein includes derivatives comprising one or more moieties that modulate or improve one or more physical, physiological or pharmaceutical property such as, but not limited to, solubiliy, permeability, uptake, biodistribution, metabolic stability, onset of action or some other druglike property, and is transformed to the bioactive or more biologically active substance as provided herein. In one embodiment, a prodrug herein has no biological activity until release of the compound or pharmaceutically acceptable salt thereof.

Thus, in one aspect provided herein is a pharmaceutical composition comprising a compound or pharmaceutically acceptable salt thereof as described herein and one or more pharmaceutically acceptable excipients.

Compounds or a stereoisomer, tautomer, or pharmaceutically acceptable salt thereof described herein can be administered by any route appropriate to the condition to be treated. Suitable routes include oral, parenteral (including subcutaneous, intramuscular, intravenous (IV), intraarterial, intradermal, intrathecal and epidural), transdermal, rectal, nasal, topical (including buccal and sublingual), vaginal, intraperitoneal, intrapulmonary and intranasal. In one embodiment, a compound or a stereoisomer, tautomer, or pharmaceutically acceptable salt thereof described herein is administered orally or by IV.

In one embodiment, compounds or a stereoisomer, tautomer, or pharmaceutically acceptable salt thereof described herein are administered as pharmaceutical compositions capable of being administered to a subject orally or parenterally. The compounds or a stereoisomer, tautomer, or pharmaceutically acceptable salt thereof described herein can be formulated for topical or parenteral use where the compound or a stereoisomer, tautomer, or pharmaceutically acceptable salt thereof is dissolved or otherwise suspended in a solution suitable for injections, suspensions, syrups, creams, ointments, gels, sprays, solutions and emulsions.

Oral administration can promote patient compliance in taking the compound (e.g. formulated as a pharmaceutical composition), thereby increasing compliance and efficacy. Oral pharmaceutical compositions comprising a compound described herein include, but are not limited to, tablets (e.g. coated, non-coated and chewable) and capsules (e.g. hard gelatin capsules, soft gelatin capsules, enteric coated capsules, and sustained release capsules). Tablets can be prepared by direct compression, by wet granulation, or by dry granulation.

### Methods of Use

The compounds, stereoisomers, tautomer, and pharmaceutically acceptable salts thereof described herein possess pharmacological properties for the treatment or prevention of bacterial infections, including bacteremia, pneumonia, meningitis, urinary tract infection, and wound infection, or a combination thereof caused by one or more bacterial pathogens. In one such embodiment, the bacterial infection is caused by one or more Acinetobacter species. In one such embodiment, the bacterial infection is caused by Acinetobacter baumannii.

In one embodiment of the compounds described herein, the compounds, stereoisomers, tautomer, and pharmaceutically acceptable salts thereof described herein possess pharmacological properties for the treatment of infections, including bacteremia, pneumonia, meningitis, urinary tract infection, and wound infection, or a combination thereof caused by bacterial pathogens. In one such embodiment, the infection is caused by one or more Acinetobacter species. In one such embodiment, the bacterial infection is caused by Acinetobacter baumannii.

The compounds, stereoisomers, tautomer, and pharmaceutically acceptable salts thereof described herein can be used as antibiotics, i.e. as antibacterial pharmaceutical ingredients suitable in the treatment or prevention of bacterial infections. In one embodiment, the compounds, stereoisomers, tautomer, and pharmaceutically acceptable salts thereof described herein are used as antibiotics in the treatment of bacterial infections caused by Acinetobacter species. In another embodiment, the compounds, stereoisomers, tautomer, and pharmaceutically acceptable salts thereof described herein are used as antibiotics in the treatment and prevention of bacterial infections caused by Acinetobacter baumannii.

The compounds, stereoisomers, tautomer, and pharmaceutically acceptable salts thereof described herein can be used, either alone or in combination with other drugs, for the treatment or prevention of infections, including bacteremia, pneumonia, meningitis, urinary tract infection, and wound infection, or a combination thereof caused by pathogens such as bacteria. In one embodiment, the compounds, stereoisomers, tautomer, and pharmaceutically acceptable salts thereof described herein can be used, either alone or in combination with other drugs, for the treatment of infections and resulting diseases caused by Acinetobacter species. In one such embodiment, the Acinetobacter species is Acinetobacter baumannii.

In one aspect, the compounds, stereoisomers, tautomer, and pharmaceutically acceptable salts thereof described herein are useful as therapeutically active substances. In a further aspect, the present invention provides a compound, stereoisomer, tautomer, or a pharmaceutically acceptable salts thereof described herein, for use as antibiotic.

In a further aspect, the present invention provides a compound, stereoisomer, tautomer, or a pharmaceutically acceptable salt thereof described herein, for use in the treatment or prevention of nosocomial infections. In one such embodiment, the nosocomial infection is bacteremia, pneumonia, meningitis, urinary tract infection, and wound infection, or a combination thereof.

In a further aspect, the present invention provides a compound, stereoisomer, tautomer, or a pharmaceutically acceptable salt thereof described herein, for use in the treatment or prevention of infection caused by Gram-negative bacteria. In one such embodiment, the infection caused by Gram-negative bacteria is bacteremia, pneumonia, meningitis, urinary tract infection, and wound infection, or a combination thereof.

In a further aspect, the present invention provides a compound, stereoisomer, tautomer, or a pharmaceutically acceptable salt thereof described herein, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound, stereoisomer, tautomer, or a pharmaceutically acceptable salt thereof described herein, as an antibiotic.

In a further aspect, the present invention provides the use of compound, stereoisomer, tautomer, or a pharmaceutically acceptable salt thereof described herein, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound, stereoisomer, tautomer, or a pharmaceutically acceptable salt thereof described herein, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In one embodiment, the infection caused by Enterococcus faecium, Staphylococcus aureus, Klebsiella pneumoniae, Acinetobacter baumannii, Pseudomonas aeruginosa, Enterobacter species or E. coli, or a combination thereof, is bacteremia, pneumonia, meningitis, urinary tract infection, and wound infection, or a combination thereof.

In a further aspect, the present invention provides a compound, stereoisomer, tautomer, or a pharmaceutically acceptable salt thereof described for use in the treatment of infections and resulting diseases, particularly bacteremia, pneumonia, meningitis, urinary tract infection, and wound infection, caused by pathogens including bacteria. In one such embodiment, the bacteria is an Acinetobacter species. In one such embodiment, the Acinetobacter species is Acinetobacter baumannii.

Further provided herein are methods comprising treating a bacterial infection. In one such embodiment is a method comprising administering to a patient having such a bacterial infection an effective amount of a compound, stereoisomer, tautomer, or pharmaceutically acceptable salt thereof to the patient. In one such embodiment, the bacterial infection is caused by an Acinetobacter species. In another such embodiment, the bacterial infection is caused by Acinetobacter baumannii.

In another embodiment is a method of treating an infection where the method comprises administering to a patient having such an infection an effective amount of a compound, stereoisomer, tautomer, or pharmaceutically acceptable salt thereof described herein in combination with one or more other agents (i.e. Antibiotics). In one such embodiment, the infection is caused by a bacterial pathogen. In one such embodiment, the infection is bacteremia, pneumonia, meningitis, urinary tract infection, or wound infection, or a combination thereof caused by a bacterial pathogen. In one embodiment, the bacterial pathogen is an Acinetobacter species. In one such embodiment, the Acinetobacter species is Acinetobacter baumannii.

Further provided herein is a method of treating nosocomial infections where the method comprises administering to a patient having a nosocomial infection an effective amount of a compound, stereoisomer, tautomer, or a pharmaceutically acceptable salt thereof described herein. In one such embodiment, the nosocomial infection is bacteremia, pneumonia, meningitis, urinary tract infection, or wound infection, or a combination thereof. In one such embodiment, the nosocomial infection is caused by a bacterial pathogen. In one embodiment, the bacterial pathogen is an Acinetobacter species. In one such embodiment, the Acinetobacter species is Acinetobacter baumannii.

Also provided herein is a method of treating infections caused by Gram-negative bacteria where the method comprises administering to a patient having an infection caused by Gram-negative bacteria a compound, stereoisomer, tautomer, or a pharmaceutically acceptable salt thereof described herein. In one such embodiment, the infection caused by Gram-negative bacteria is bacteremia, pneumonia, meningitis, urinary tract infection, and wound infection, or a combination thereof. In another such embodiment, the Gram-negative bacteria is an Acinetobacter species. In one such embodiment, the Acinetobacter species is Acinetobacter baumannii.

Further provided herein is a method for treating infections caused by Enterococcus faecium, Staphylococcus aureus, Klebsiella pneumoniae, Acinetobacter baumannii, Pseudomonas aeruginosa, Enterobacter species or E. coli, or a combination thereof in a patient having such an infection, the method comprising administering to the patient an effective amount of a compound, stereoisomer, tautomer, or a pharmaceutically acceptable salt thereof described herein. In one such embodiment, the infection caused by Enterococcus faecium, Staphylococcus aureus, Klebsiella pneumoniae, Acinetobacter baumannii, Pseudomonas aeruginosa, Enterobacter species or E. coli, or a combination thereof, is bacteremia, pneumonia, meningitis, urinary tract infection, and wound infection, or a combination thereof.

### Co-Administration of Agents

The compound, stereoisomer, tautomer, or a pharmaceutically acceptable salt thereof as described herein may be employed alone or in combination with other agents for treatment. For example, the second agent of the pharmaceutical combination formulation or dosing regimen may have complementary activities to such compound such that they do not adversely affect each other. The second agent and a compound, stereoisomer, tautomer, or a pharmaceutically acceptable salt thereof as described herein may be administered together in a unitary pharmaceutical composition or separately. In one embodiment a compound, stereoisomer, tautomer, or a pharmaceutically acceptable salt thereof as described herein can be co-administered with an antibiotic, in particular with an antibiotic for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

Typically, any agent that has antimicrobial activity may be co-administered. Particular examples of such agents are Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified e.g. in WO 2017072062 A1, WO 2019185572 A1 and WO 2019206853 A1, and Macrolides (erythromycin).

In one aspect, is a pharmaceutical composition described herein, further comprising an additional therapeutic agent.

In one aspect, is a pharmaceutical combination comprising a compound, stereoisomer, tautomer, or a pharmaceutically acceptable salt thereof described herein and an additional therapeutic agent. In one such embodiment, the additional therapeutic agent is an antibiotic agent. In another such embodiment, the additional therapeutic agent is an antibiotic agent that is useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof. In one such embodiment, the additional therapeutic agent is an antibiotic agent selected from Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified in WO 2017072062 A1, WO 2019185572 A1 and WO 2019206853 A1, and Macrolides (erythromycin).

### Articles of Manufacture

Also provided herein are articles of manufacture, or “kit”, containing materials useful as an antibiotic agent that is useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof. In one embodiment, the kit comprises a container comprising compound or stereoisomer, tautomer, or pharmaceutically acceptable salt thereof described herein. The kit may further comprise a label or package insert on or associated with the container. Suitable containers include, for example, bottles, vials, syringes, blister pack, etc. The container may be formed from a variety of materials such as glass or plastic. The container may hold a compound or a pharmaceutically acceptable salt thereof described herein or a formulation thereof which is effective for treating the condition and may have a sterile access port (for example, the container may be an intravenous solution bag or a vial having a stopper pierceable by a hypodermic injection needle). At least one active agent in the composition is a compound or a pharmaceutically acceptable salt thereof described herein. Alternatively, or additionally, the article of manufacture may further comprise a second container comprising a pharmaceutical diluent, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In another embodiment, the kits are suitable for the delivery of solid oral forms of a compound or a pharmaceutically acceptable salt thereof described herein, such as tablets or capsules. Such a kit can include a number of unit dosages. An example of such a kit is a “blister pack”. Blister packs are well known in the packaging industry and are widely used for packaging pharmaceutical unit dosage forms.

### Exemplary Embodiments

Provided below are exemplary embodiments of the invention described herein.

Embodiment 1. A compound of formula (I) or (II):

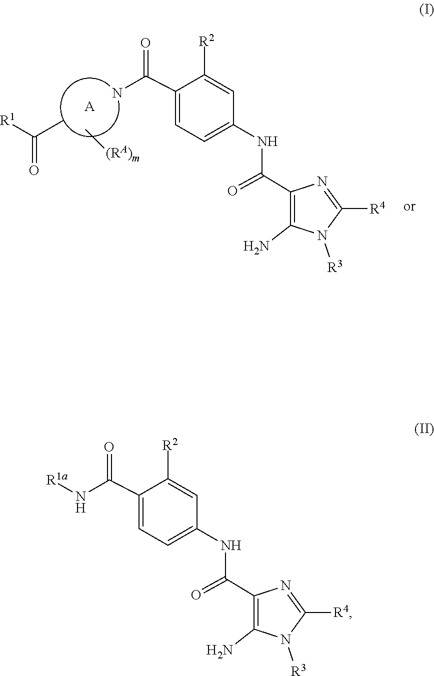

or a stereoisomer or pharmaceutically acceptable salt thereof, wherein
Ring A is 4- to 8-membered R^{A}-substituted or unsubstituted heterocyclyl;
R^{A} is halogen, NH₂, OH, unsubstituted C₁₋₃alkyl, or unsubstituted C₁₋₃haloalkyl;
m is 0 or 1;
R¹ is N(R⁶)₂, R⁶-substituted or unsubstituted C₁₋₆alkyl, R⁶-substituted or unsubstituted C₁₋₃haloalkyl, R⁶-substituted or unsubstituted C₃₋₆cycloalkyl, or R⁶-substituted or unsubstituted 4- to 8-membered heterocyclyl comprising 1 or 2 heteroatoms independently selected from N, O, S, or SO₂;
each R⁶ is independently hydrogen, halogen, CH(═NH)(NH₂), (═NH), N(R⁷)₂, OR⁷, NHC(O)R⁷, C(O)OR⁷, R⁷-substituted or unsubstituted C₁₋₆alkyl, R⁷-substituted or unsubstituted C₁₋₆haloalkyl, R⁷-substituted or unsubstituted C₃₋₆ cycloalkyl, or R⁷-substituted or unsubstituted 4- to 6-membered heterocyclyl; or
wherein two or more R⁶ groups together form a C₁₋₂ bridge
each R⁷ is hydrogen, halo, OH, COOH, (═NH), NH₂, unsubstituted C₁₋₃alkyl, unsubstituted C₁₋₃alkyoxy, unsubstituted C₁₋₃alkylaryl, unsubstituted C₁₋₆alkyl heterocyclyl, or unsubstituted 4- to 6-membered heterocyclyl;
R^{1a} is a moiety of formula:

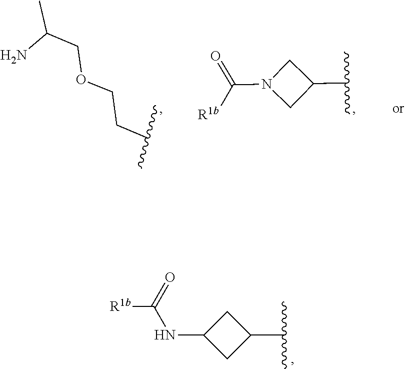

or a stereoisomer thereof;
R^{1b} is unsubstituted 5- to 7-membered heterocyclyl;
R² is hydrogen, halogen, unsubstituted C₁₋₃alkyl, or unsubstituted C₁₋₃haloalkyl;
R³ is a moiety of formula:

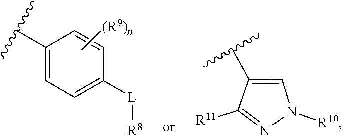

or a stereoisomer thereof
L is absent, —O—, or —NR^{8b}—;
R⁸ is R^{8a}-substituted or unsubstituted C₁₋₆alkyl, R^{8a}-substituted or unsubstituted C₁₋₃haloalkyl, or R^{8a}-substituted or unsubstituted C₃₋₆cycloalkyl;
wherein when L is absent R⁸ is

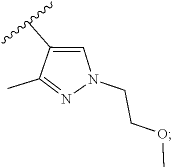

R^{8a} is unsubstituted C₁₋₃alkyl, OH, or OR^{8b} wherein R^{8b} is unsubstituted C₁₋₃alkyl;
R^{8b} is hydrogen or unsubstituted C₁₋₃alkyl;
R⁹ is halo, CN, unsubstituted C₁₋₆alkyl, unsubstituted C₁₋₃haloalkyl, unsubstituted C₁₋₆alkoxy, or unsubstituted cyclopropyl;
n is 0, 1, or 2;
R¹⁰ is R^{10A}-substituted or unsubstituted C₁₋₆alkyl, R^{10A}-substituted or unsubstituted C₂₋₆alkenyl, R^{10A}-substituted or unsubstituted C₂₋₆alkynl, R^{10A}, substituted or unsubstituted C₁₋₃haloalkyl, or R^{10A}-substituted or unsubstituted C₃₋₆cycloalkyl;
R^{10A} is halo, CN, OH, OCH₃, unsubstituted C₁₋₃haloalkyl, or R^{10B}-substituted or unsubstituted C₃₋₅cycloalkyl, or wherein 2 R^{10A} groups together form a R^{10B}, substituted or unsubstituted C₃₋₄spirocycle;
R^{10B} is halo or unsubstituted C₁₋₃haloalkyl;
R¹¹ is halo or unsubstituted C₁₋₃haloalkyl; and
R⁴ is hydrogen, halo, or unsubstituted C₁₋₃alkyl.

Embodiment 1a. A compound of formula (I) or (II):

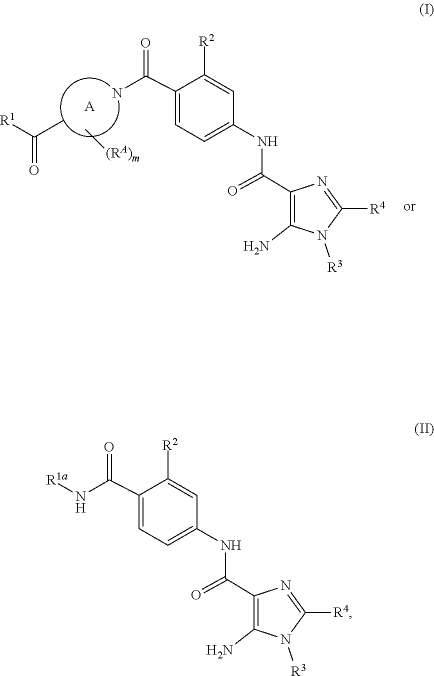

or a stereoisomer or pharmaceutically acceptable salt thereof, wherein
or a stereoisomer or pharmaceutically acceptable salt thereof, wherein
Ring A is 4- to 8-membered R^{A}-substituted or unsubstituted heterocyclyl;
R^{A} is halogen, NH₂, OH, unsubstituted C₁₋₃alkyl, or unsubstituted C₁₋₃haloalkyl;
m is 0 or 1;
R¹ is N(R⁶)₂, R⁶-substituted or unsubstituted C₁₋₆alkyl, R⁶-substituted or unsubstituted C₁₋₃haloalkyl, R⁶-substituted or unsubstituted C₃₋₆cycloalkyl, or R⁶-substituted or unsubstituted 4- to 8-membered heterocyclyl comprising 1 or 2 heteroatoms independently selected from N, O, S, or SO₂;
each R⁶ is independently hydrogen, halogen, CH(═NH)(NH₂), (═NH), N(R⁷)₂, OR⁷, NHC(O)R⁷, C(O)OR⁷, R⁷-substituted or unsubstituted C₁₋₆alkyl, R⁷-substituted or unsubstituted C₁₋₆haloalkyl, R⁷-substituted or unsubstituted C₃₋₆ cycloalkyl, or R⁷-substituted or unsubstituted 4- to 6-membered heterocyclyl; or
wherein two or more R⁶ groups together form a C₁₋₂ bridge;
each R⁷ is hydrogen, halo, OH, COOH, (═NH), NH₂, unsubstituted C₁₋₃alkyl, unsubstituted C₁₋₃alkyoxy, unsubstituted C₁₋₃alkylaryl, unsubstituted C₁₋₆alkyl heterocyclyl, or unsubstituted 4- to 6-membered heterocyclyl;
R^{1a} is a moiety of formula:

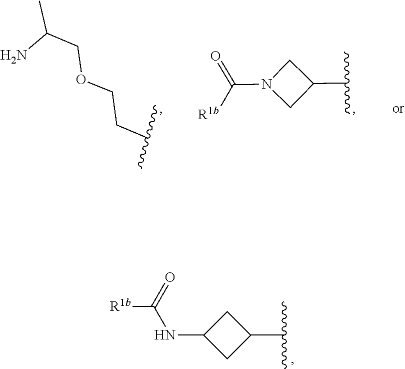

or a stereoisomer thereof;
R^{1b} is O—C₁₋₃alkyaryl, unsubstituted 5- to 7-membered heterocyclyl;
R² is hydrogen, halogen, unsubstituted C₁₋₃alkyl, or unsubstituted C₁₋₃haloalkyl;
R³ is a moiety of formula:

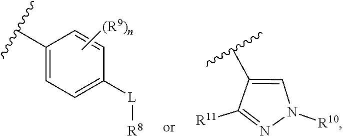

or a stereoisomer thereof
L is absent, —O—, or —NR^{8b}—;
R⁸ is R^{8a}-substituted or unsubstituted C₁₋₆alkyl, R^{8a}-substituted or unsubstituted C₁₋₃haloalkyl, or R^{8a}-substituted or unsubstituted C₃₋₆cycloalkyl;
wherein when L is absent R⁸ is

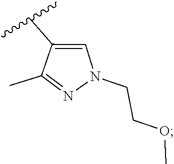

R^{8a} is unsubstituted C₁₋₃alkyl, OH, or OR^{8b} wherein R^{8b} is unsubstituted C₁₋₃alkyl;
R^{8b} is hydrogen or unsubstituted C₁₋₃alkyl;
R⁹ is halo, CN, unsubstituted C₁₋₆alkyl, unsubstituted C₁₋₃haloalkyl, unsubstituted C₁₋₆alkoxy, or unsubstituted cyclopropyl;
n is 0, 1, or 2;
R¹⁰ is R^{10A}-substituted or unsubstituted C₁₋₆alkyl, R^{10A}-substituted or unsubstituted C₂₋₆alkenyl, R^{10A}-substituted or unsubstituted C₂₋₆alkynl, R^{10A}, substituted or unsubstituted C₁₋₃haloalkyl, or R^{10A}-substituted or unsubstituted C₃₋₆cycloalkyl;
R^{10A} is halo, CN, OH, OCH₃, unsubstituted C₁₋₃haloalkyl, or R^{10B}-substituted or unsubstituted C₃₋₅cycloalkyl, or wherein 2 R^{10A} groups together form a R^{10B}-substituted or unsubstituted C₃₋₄spirocycle;
R^{10B} is halo or unsubstituted C₁₋₃haloalkyl;
R¹¹ is halo or unsubstituted C₁₋₃haloalkyl; and
R⁴ is hydrogen, halo, or unsubstituted C₁₋₃alkyl.

Embodiment 2. The compound of embodiment 1 wherein the compound comprises formula (I).

Embodiment 3. The compound of embodiment 1 or 2, wherein Ring A is a moiety of formula:

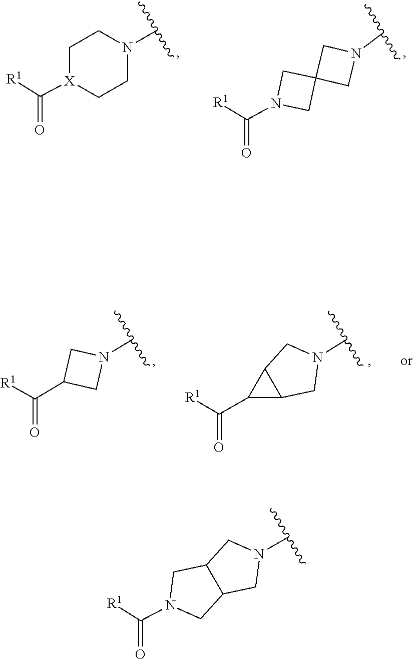

or a stereoisomer thereof, wherein
X is N or CR^{x};
R^{x} is hydrogen or halo; and
p is an integer of 0, 1, 2, 3, or 4.

Embodiment 4. The compound of any one of embodiments 1 to 3, wherein the compound of formula (I) comprises formula (Ia) as described herein or a stereoisomer or pharmaceutically acceptable salt thereof, wherein X is N, CH or CF.

Embodiment 5. The compound of any one of embodiments 1 to 4, wherein the compound of formula (I) comprises formula (Ia1) or (Ia2) as described herein, or a stereoisomer or pharmaceutically acceptable salt thereof.

Embodiment 6. The compound of any one of embodiments 1 to 5, wherein R¹ is R⁶-substituted or unsubstituted 4- to 8-membered heterocyclyl comprising 1 or 2 heteroatoms independently selected from N, or O.

Embodiment 7. The compound of any one of embodiments 1 to 6, wherein R¹ is a moiety of formula:

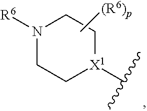

or a stereoisomer thereof, wherein X¹ is N or CH.

Embodiment 8. The compound of any one of embodiments 1 to 7, wherein R¹ is a moiety of formula:

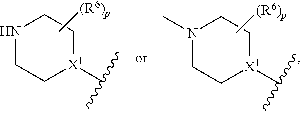

or a stereoisomer thereof, wherein X¹ is N or CH.

Embodiment 9. The compound of any one of embodiments 1 to 6, wherein R¹ is R⁶-substituted or unsubstituted 4- to 8-membered heterocyclyl comprising at least one N ring heteroatom.

Embodiment 10. The compound of any one of embodiments 1-6 or 9, wherein R¹ is R⁶-substituted or unsubstituted piperazinyl.

Embodiment 11. The compound of embodiment 10, wherein R¹ is a moiety of formula:

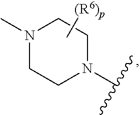

or a stereoisomer thereof, wherein R⁶ is oxo or halo (F), and p is 1 or 2.

Embodiment 12. The compound of embodiment 10, wherein R¹ is a moiety of formula:

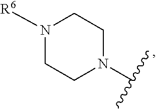

or a stereoisomer thereof.

Embodiment 13. The compound of any one of embodiments 1-6 or 9-10, wherein R¹ is unsubstituted piperazinyl.

Embodiment 14. The compound of embodiment 13, wherein R¹ is a moiety of formula:

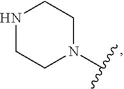 [SMILES: *N1CCNCC1]

or a stereoisomer thereof.

Embodiment 15. The compound of any one of embodiments 1-6 or 9, wherein R¹ is R⁶-substituted or unsubstituted azetidinyl, R⁶-substituted or unsubstituted pyrrolidinyl, or R⁶-substituted or unsubstituted piperidinyl.

Embodiment 16. The compound of any one of embodiments 1-6 or 9, wherein R⁶ is independently hydrogen, halogen, OR⁷, R⁷-substituted or unsubstituted C₁₋₆alkyl, or R⁷-substituted or unsubstituted C₁₋₆haloalkyl.

Embodiment 17. The compound of any one of embodiments 1-6, 9-10, 13 or 15, wherein R⁶ is halo.

Embodiment 18. The compound of any one of embodiments 1-6, 9-10, 13 or 15-16, wherein R⁶ is F.

Embodiment 19. The compound of any one of embodiments 15 to 18, wherein R¹ is a moiety of formula:

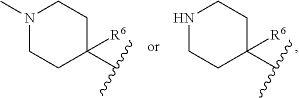

or a stereoisomer thereof.

Embodiment 20. The compound of any one of embodiments 15 to 18, wherein R¹ is a moiety of formula:

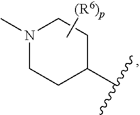

or a stereoisomer thereof, wherein R⁶ is oxo or halo (F), and p is 1 or 2.

Embodiment 21. The compound of any one of embodiments 15 to 18, wherein R¹ is R¹ is a moiety of formula:

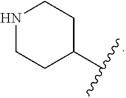 [SMILES: *C1CCNCC1]

Embodiment 22. The compound of any one of embodiments 1-6 or 9, wherein R¹ is a moiety of formula

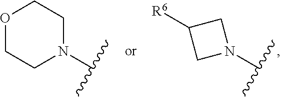

or a stereoisomer thereof.

Embodiment 23. The compound of any one of embodiments 1 to 6, wherein R¹ is a moiety of formula:

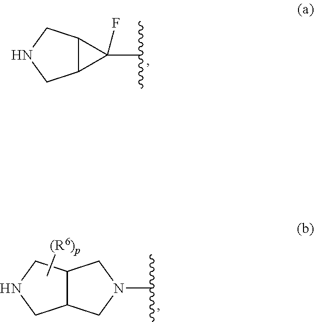

or a stereoisomer thereof.

Embodiment 24. The compound of embodiment 23, wherein p is 1 and R⁶ is halo.

Embodiment 25. The compound of embodiment 23, wherein the moiety is of formula (a), p is 0.

Embodiment 26. The compound of embodiment 23, wherein the moiety is of formula (a), p is 1 and R⁶ is halo.

Embodiment 27. The compound of any one of embodiments 1-6 or 9, wherein R¹ is a moiety of formula:

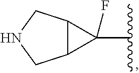 [SMILES: *C1(F)C2CNCC21]

or a stereoisomer thereof.

Embodiment 28. The compound of any one of embodiments 1-6 or 9, wherein R¹ is a moiety of formula:

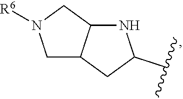

or a stereoisomer thereof, wherein R⁶ is hydrogen.

Embodiment 29. The compound of embodiment 1 or 22, wherein R⁶ is halo.

Embodiment 30. The compound of any one of embodiments 1-6 or 9, wherein R⁶ is hydrogen.

Embodiment 31. The compound of embodiment 1 having formula (II).

Embodiment 32. The compound of embodiment 1 or 31, wherein Ria is a moiety of formula:

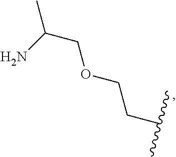 [SMILES: *CCOCC(C)N]

or a stereoisomer thereof.

Embodiment 33. The compound of embodiment 1 or 31, wherein Ria is a moiety of formula:

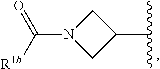

or a stereoisomer thereof.

Embodiment 34. The compound of embodiment 1 or 31, wherein Ria is a moiety of formula:

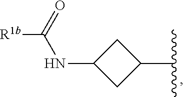

or a stereoisomer thereof.

Embodiment 35. The compound of any one of embodiments 1-6 or 9-34, wherein R³ is a moiety of formula:

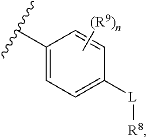

or a stereoisomer thereof.

Embodiment 36. The compound of embodiment 1 or 35, wherein L is —O—.

Embodiment 37. The compound of embodiment 1 or 35, wherein L is —NH—.

Embodiment 38. The compound of any one of embodiments 35 to 37, wherein R⁸ is unsubstituted haloalkyl.

Embodiment 39. The compound of any one of embodiments 35 to 37, wherein R⁸ is unsubstituted C₁₋₃alkyl.

Embodiment 40. The compound of any one of embodiments 35 to 37, wherein R⁸ is unsubstituted cyclopropyl.

Embodiment 41. The compound of embodiment 35, wherein L is L is —NR^{8b}— wherein R^{8b} is CH₃.

Embodiment 42. The compound of embodiment 1 or 35, wherein R³ is a moiety of formula:

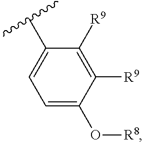

or a stereoisomer thereof.

Embodiment 43. The compound of embodiment 42, wherein R⁸ is CH₂F, CHF₂, CH₃, unsubstituted cyclopropyl.

Embodiment 44. The compound of any one of embodiments 1-6, 9-35 or 42, wherein R³ is a moiety of formula:

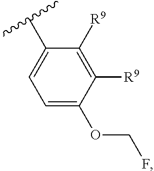

or a stereoisomer thereof.

Embodiment 45. The compound of any one of embodiments 35 to 44, wherein R⁹ is independently halo, CH₃, CF₃, CH₂F, or CHF₂.

Embodiment 46. The compound of any one of embodiments 35 to 44, wherein R⁹ is halo.

Embodiment 47. The compound of any one of embodiments 35 to 44, wherein R⁹ is F.

Embodiment 48. The compound of any one of embodiments 1-6 or 9-34, wherein R³ is a moiety of formula:

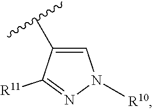

or a stereoisomer thereof.

Embodiment 49. The compound of embodiment 1 or 48, wherein R¹¹ is CF₃.

Embodiment 50. The compound of any one of embodiments 1 or 48-49, wherein R¹⁰ is R^{10A}-substituted or unsubstituted C₁₋₆alkyl, R^{10A}-substituted or unsubstituted C₂₋₆alkenyl, or R^{10A}-substituted or unsubstituted C₂₋₆alkynl.

Embodiment 51. The compound of embodiment 50, wherein R^{10A} is independently halo.

Embodiment 52. The compound of embodiment 1 or 48, wherein R¹⁰ is R^{10A}-substituted or unsubstituted C₂₋₆alkenyl, wherein each R^{10A} is independently halo, CN, OH, OCH₃, or unsubstituted C₃₋₅cycloalkyl.

Embodiment 53. The compound of embodiment 1 or 48, wherein R¹⁰ is unsubstituted cyclobutyl or unsubstituted cyclopropyl.

Embodiment 54. The compound of embodiment 1 or 48, wherein R³ is a moiety of formula:

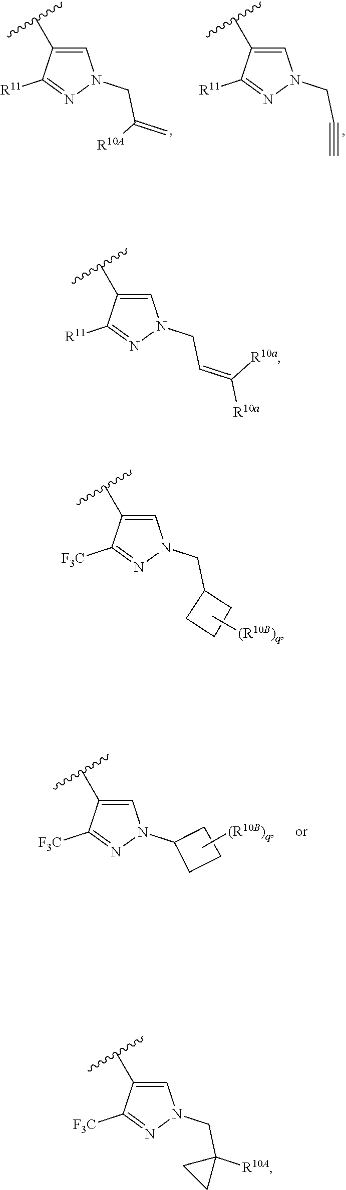

or a stereoisomer thereof, wherein q is an integer of 0, 1, or 2.

Embodiment 55. The compound of embodiment 54, wherein each R^{10A} is independently halo.

Embodiment 56. The compound of embodiment 54, wherein each R¹¹ is F or Cl.

Embodiment 57. The compound of any one of embodiments 1 to 56, wherein R² is hydrogen.

Embodiment 58. The compound of any one of embodiments 1-6 or 9-57, wherein R² is unsubstituted C₁₋₃alkyl.

Embodiment 59. The compound of any one of embodiments 1-6 or 9-57, wherein R² is halo.

Embodiment 60. The compound of any one of embodiments 1-6 or 9-57, wherein R² is C₁.

Embodiment 61. The compound of any one of embodiments 1-6 or 9-57, wherein R² is CH₃.

Embodiment 62. The compound of any one of embodiments 1-6 or 9-57, wherein R² is unsubstituted C₁₋₃haloalkyl.

Embodiment 63. The compound of any one of embodiments 1-6 or 9-57, wherein R² is CH₂F, CHF₂, or CF₃.

Embodiment 64. The compound of any one of embodiments 1-6 or 9-57, wherein R⁴ is hydrogen.

Embodiment 65. The compound of any one of embodiments 1-6 or 9-57, wherein R⁴ is halo.

Embodiment 66. The compound of any one of embodiments 1-6 or 9-57, wherein R⁴ is C₁.

Embodiment 67. The compound of any one of embodiments 1-6 or 9-57, wherein R⁴ is CH₃.

Embodiment 68. The compound of any one of embodiments 1-6 or 9-57, wherein the compound of formula (I) has formula (Id) as described herein, or a stereoisomer or pharmaceutically acceptable salt thereof.

Embodiment 69. The compound of embodiment 68, wherein X is N or CH.

Embodiment 70. The compound of embodiment 1, wherein the compound of formula (I) has formula (Ie) as described herein or a stereoisomer or pharmaceutically acceptable salt thereof, wherein,
X is N or CH;
X¹ is CH;
R² is halo; and
R⁴ is hydrogen.

Embodiment 71. A compound of Table 1 or a stereoisomer or pharmaceutically acceptable salt thereof.

Embodiment 72. A pharmaceutical composition comprising a compound or a pharmaceutically acceptable salt thereof of any one of embodiments 1-6, 9-57, 59-67 or 71 and one or more pharmaceutically acceptable excipients.

Embodiment 73. A compound according to any one of embodiments 1-6, 9-57, 59-67 or 71, or a pharmaceutically acceptable salt thereof, for use as antibiotic.

Embodiment 74. A compound according to any one of embodiments 1-6, 9-57, 59-67 or 71, or a pharmaceutically acceptable salt thereof, for use in the treatment of infection caused by Gram-negative bacteria.

Embodiment 75. The compound for use according to embodiment 74, wherein the Gram-negative bacteria are Klebsiella pneumoniae, Acinetobacter baumannii, Pseudomonas aeruginosa, Enterobacter species or E. coli.

Embodiment 76. The compound for use according to embodiment 74, wherein the Gram-negative bacteria are Acinetobacter baumannii.

Embodiment 77. A compound according to any one of embodiments 1-6, 9-57, 59-67 or 71, or a pharmaceutically acceptable salt thereof, for use in the treatment of infections caused by Enterococcus faecium, Staphylococcus aureus, Klebsiella pneumoniae, Acinetobacter baumannii, Pseudomonas aeruginosa, Enterobacter species or E. coli, or a combination thereof.

Embodiment 78. A method of treating an infection caused by Gram-negative bacteria in a patient having such an infection, the method comprising administering to the patient an effective amount of a compound of any one of embodiments 1-6, 9-57, 59-67 or 71 or a stereoisomer, tautomer, or pharmaceutically acceptable salt thereof.

Embodiment 79. The method of embodiment 78, wherein the Gram-negative bacteria are Klebsiella pneumoniae, Acinetobacter baumannii, Pseudomonas aeruginosa, Enterobacter species or E. coli.

Embodiment 80. The method of embodiment 78, wherein the Gram-negative bacteria are Acinetobacter baumannii.

Embodiment 81. A method of treating an infection caused by Enterococcus faecium, Staphylococcus aureus, Klebsiella pneumoniae, Acinetobacter baumannii, Pseudomonas aeruginosa, Enterobacter species or E. coli, or a combination thereof, in a patient having such infection, the method comprising administering to the patient a compound of any one of embodiments 1-6, 9-57, 59-67 or 71 or a stereoisomer, tautomer or pharmaceutically acceptable salt thereof.

### Examples

The following Examples are presented by way of illustration, not limitation.

### Intermediates

### Intermediate 1

### 2-amino-2-cyano-acetic acid ethyl ester

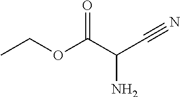

To a suspension of (2E)-2-cyano-2-hydroximino-acetic acid ethyl ester (10 g, 70 mmol, 1.0 eq) in water (60 mL) was added slowly saturated sodium bicarbonate solution in water (29.56 g, 29.56 mL, 351.84 mmol, 5 eq). Attention: vigorous gas evolution occurs. Then sodium dithionite (36.75 g, 211.1 mmol, 3.000 eq) was added in portions over a period of 40 min (exothermic, max temp 41° C.). After 1 h the solution was extracted with dichloromethane (4×50 ml). The combined organic layers were washed with brine, dried over magnesium sulfate, filtered and concentrated under reduced pressure to afford the title compound (2.33 g, 31%), which was used immediately without further purification. Beware: Compound is not stable at room temperature or in the refrigerator. Stored under Argon in the deep-freezer.

### Intermediate 2

### ethyl 5-amino-1-[2,3-difluoro-4-(fluoromethoxy)phenyl]imidazole-4-carboxylate

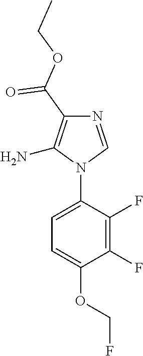 [SMILES: CCOC(=O)c1ncn(-c2ccc(OCF)c(F)c2F)c1N]

### Step 1: 2,3-difluoro-4-(fluoromethoxy)aniline

To a solution of 4-amino-2,3-difluorophenol (25.0 g, 172.28 mmol, 1.0 eq) and potassium carbonate (28.57 g, 206.7 mmol, 1.2 eq) in MeCN (400 mL) was added bromofluoromethane (1.51 mL, 23.8 mmol, 1.15 eq) at 0° C. The mixture was stirred at 20° C. for 16 hours to give a suspension. The mixture was concentrated under vacuum. The residue was diluted with saturated aq. NH₄Cl (200 mL) and then extracted with EA (3×200 mL). The combined organic layers were washed with brine (300 mL), dried over sodium sulphate, and filtered. The filtrate was concentrated under vacuum.

The residue was purified by silica gel chromatography using petrol ether/ethyl acetate as eluent. The corresponding fractions were concentrated under vacuum to afford the title compound (23.0 g, 130 mmol, 75.4% yield) as colorless oil, which was used without further purification. MS: m/z=178.0 [M+H]⁺, ESI pos.

### Step 2: ethyl 5-amino-1-[2,3-difluoro-4-(fluoromethoxy)phenyl]imidazole-4-carboxylate

To a solution of ethyl 2-amino-2-cyanoacetate 4-methylbenzene-1-sulfonic acid salt (14.89 g, 49.59 mmol, 1.25 eq) and triethylamine (6.89 mL, 49.4 mmol, 1.25 eq) in trimethyl orthoformate (150.0 mL, 1371.09 mmol, 34.69 eq). The mixture was stirred at 85° C. for 13 hours. Then the mixture was concentrated under reduced pressure. The residue was dissolved in ACN (100 mL), 2,3-difluoro-4-(fluoromethoxy)aniline (7.0 g, 40 mmol, 1.0 eq) was added and the mixture was stirred at 90° C. for 16 hours. The mixture was concentrated under vacuum. The residue was diluted with water (100 mL) and saturated aq. NaHCO₃ (50 mL). The mixture was extracted with EA (3×150 mL). The combined organic layers were was washed with brine (200 mL), dried over sodium sulfate, filtered, the filtrate was concentrated under vacuum. The residue was purified by silica gel chromatography using PE/EA as eluent to afford the title compound (5.0 g, 16 mmol, 40% yield). MS: 316.0 [M+H]⁺, ESI pos.

### Intermediate 3

### 5-amino-1-[2-(difluoromethyl)-3-fluoro-4-(fluoromethoxy)phenyl]imidazole-4-carboxylic acid ethyl ester

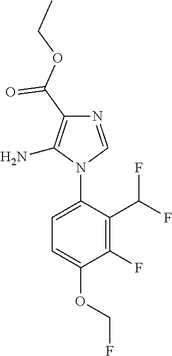 [SMILES: CCOC(=O)c1ncn(-c2ccc(OCF)c(F)c2C(F)F)c1N]

### Step 1:2-fluoro-3-methyl-4-nitro-phenol

To a solution of KOH (2.36 g, 42.02 mmol, 2.500 eq) in water (18 mL) and dimethyl sulfoxide (9 mL) was added 1,2-difluoro-3-methyl-4-nitro-benzene (3 g, 17 mmol, 1.0 eq). The solution was heated to 90° C. and stirred overnight. The reaction was cooled to room temperature and the pH was acidified with 3 M HCl (18.15 g, 15.13 mL, 45.38 mmol, 2.700 eq) and extracted 3 times with ethyl acetate. The combined organic layers were dried over magnesium sulfate, filtered and concentrated under reduced pressure to afford 3.26 g. The crude product was purified by silica gel chromatography using heptane/ethyl acetate 1:1 as eluent to afford the title compound (2.86 g, 80% yield) with a purity of approx 85%, which was used in the next step without any further purification.

¹H NMR (300 MHz, DMSO-d6) δ=11.30 (br s, 1H), 7.84 (dd, J=1.7, 9.2 Hz, 1H), 6.97 (t, J=8.9 Hz, 1H), 2.44 (d, J=2.8 Hz, 3H)

### Step 2: 2-fluoro-1-(fluoromethoxy)-3-methyl-4-nitrobenzene

To a solution of 2-fluoro-3-methyl-4-nitrophenol (2.86 g, 14.2 mmol, 1.00 eq) in acetonitrile (28 mL) was added DIEA (5.51 g, 7.44 mL, 42.6 mmol, 3.00 eq) and fluoro (iodo) methane (4.64 g, 1.96 mL, 28.4 mmol, 2.00 eq). The tube was sealed and heated to 50° C. for 4 h. The solvent was evaporated. The residue was diluted with ethyl acetate and washed with water and brine. The aq. layers were extracted additional 2 times with ethyl acetate. The combined organic layers were dried over magnesium sulfate, filtered and concentrated under reduced pressure to afford the title compound (3.38 g, 93.6% yield) with a purity of approx. 80%.

¹H NMR (300 MHz, CHLOROFORM-d) δ=7.88 (dd, J=2.0, 9.3 Hz, 1H), 7.17 (dt, J=0.7, 8.5 Hz, 1H), 5.82 (d, J=53.0 Hz, 2H), 2.57 (d, J=2.6 Hz, 3H)

### Step 3: 3-(bromomethyl)-2-fluoro-1-(fluoromethoxy)-4-nitrobenzene

To a solution of 2-fluoro-1-(fluoromethoxy)-3-methyl-4-nitro-benzene (2185 mg, 9.68 mmol, 1.000 eq) in carbon tetrachloride (27.55 mL) was added N-bromosuccinimide (2.58 g, 14.5 mmol, 1.50 eq) and benzoyl peroxide (312.65 mg, 968.05 μmol, 0.100 eq). The mixture was heated to 80° C. for 2 h. Again, benzoyl peroxide (312.65 mg, 968.05 μmol, 0.100 eq) and N-bromosuccinimide (2.58 g, 14.5 mmol, 1.50 eq) were added and stirred for 2 hours. Then again, N-bromosuccinimide (2.58 g, 14.52 mmol, 1.500 eq) and benzoyl peroxide (312.65 mg, 968.05 μmol, 0.100 eq) were added and stirring continued at 80° C. overnight. The reaction was cooled to room temperature and then diluted with dichloromethane and water. The aqueous layer was extracted 3 times with dichloromethane. The combined organic layers were washed again with water and brine, dried over magnesium sulfate, filtered and concentrated under reduced pressure. The residue as drypack on isolute HM-N adsorbent was purified by silica gel chromatography using heptane/ethyl acetate as eluent to afford the title compound (2.22 g, 69% yield) with a purity of approx. 85%.

¹H NMR (300 MHz, CHLOROFORM-d) δ=7.99 (dd, J=2.0, 9.3 Hz, 1H), 7.28 (ddd, J=1.2, 8.0, 9.2 Hz, 1H), 5.82 (d, J=53.0 Hz, 2H), 4.89 (d, J=2.4 Hz, 2H)

### Step 4: 2-fluoro-3-(fluoromethoxy)-6-nitrobenzaldehyde

To a solution of 3-(bromomethyl)-2-fluoro-1-(fluoromethoxy)-4-nitrobenzene (2.22 g, 6.69 mmol, 1.00 eq) in acetonitrile (26.45 mL) was added molecular sieves 4 Å (2.15 g) and 4-methylmorpholine N-oxide (1.94 g, 16.1 mmol, 2.40 eq). The reaction was stirred at 25° C. for 20 hours. The reaction was filtered and the solid was discarded and the mother liquor was concentrated. The residue was diluted with ethyl acetate and washed with water and brine. The aqueous layers were back-extracted 2 times with ethyl acetate. The combined organic layers were dried over magnesium sulfate, filtered and concentrated under reduced pressure. The residue was suspended in TBME, filtered, and the solid collected to afford the title compound (492 mg).

The mother liquor as drypack on isolute HM-N adsorbent was purified by silica gel chromatography using heptane/ethyl acetate as eluent to afford another batch of title compound (552 mg). Both batches were combined and used in subsequent steps.

¹H NMR (300 MHz, CHLOROFORM-d) δ=10.32 (s, 1H), 8.03 (dd, J=1.8, 9.1 Hz, 1H), 7.44 (ddd, J=1.2, 7.7, 9.0 Hz, 1H), 5.84 (d, J=52.6 Hz, 2H)

### Step 5: 3-(difluoromethyl)-2-fluoro-1-(fluoromethoxy)-4-nitrobenzene

To a solution of 2-fluoro-3-(fluoromethoxy)-6-nitrobenzaldehyde (1044 mg, 4.71 mmol, 1.000 eq) in 2.7 M [bis(2-methoxyethyl)amino]sulfur trifluoride solution in toluene (10.47 g, 8.73 mL, 23.56 mmol, 5.000 eq) was added MeOH (15.1 mg, 19.1 μL, 471 μmol, 0.100 eq). The reaction was heated to 50° C. for 2 hours. The reaction was cooled to room temperature and then poured onto ice/water and the resulting mixture extracted 3 times with ethyl acetate. The combines organic layers were washed with brine, dried over magnesium sulfate, filtered and concentrated under reduced pressure. The residue as drypack on isolute HM-N adsorbent was purified by silica gel chromatography using ethyl acetate/heptane as eluent to afford the title compound (1021 mg; 91% yield).

¹H NMR (300 MHz, CHLOROFORM-d) δ=7.90 (td, J=0.9, 9.2 Hz, 1H), 7.49-7.08 (m, 2H), 5.83 (d, J=52.8 Hz, 2H)

### Step 6: [2-(difluoromethyl)-3-fluoro-4-(fluoromethoxy)phenyl]amine

3-(difluoromethyl)-2-fluoro-1-(fluoromethoxy)-4-nitrobenzene (1016 mg, 4.25 mmol, 1.000 eq) was dissolved in ethanol (26 mL) and water (8 mL). zinc (2.78 g, 42.5 mmol, 10.0 eq) and ammonium chloride (4.55 g, 85.0 mmol, 20.0 eq) were added. The grayish suspension was heated to 50° C. and stirred for 20 hours. The reaction was cooled to room temperature, was filtered, and washed with ethyl acetate. The mother liquor was concentrated under reduced pressure and the residue was extracted using ethyl acetate and water. The organic phase was washed with brine, dried with MgSO₄, filtered and concentrated under reduced pressure. The crude material as drypack on isolute HM-N adsorbent was purified by silica gel chromatography using heptane/dichloromethane as eluent to afford the title compound (452 mg, 51% yield).

¹H NMR (300 MHz, CHLOROFORM-d) δ=7.11 (dt, J=1.1, 8.9 Hz, 1H), 6.99 (t, J=53.8 Hz, 1H), 6.42 (dd, J=1.7, 9.0 Hz, 1H), 5.57 (d, J=54.4 Hz, 2H), 4.25 (br s, 2H).

### Step 7: 5-amino-1-[2-(difluoromethyl)-3-fluoro-4-(fluoromethoxy)phenyl]imidazole-4-carboxylic acid ethyl ester

To 1 M solution of 2-amino-2-cyanoacetic acid ethyl ester in CH₃CN (intermediate 1, 2.59 mL, 2.59 mmol, 1.20 eq) was added a solution of [2-(difluoromethyl)-3-fluoro-4-(fluoromethoxy)phenyl]amine (452 mg, 2.16 mmol, 1.00 eq) in acetonitrile (3 mL). The reaction mixture was stirred at 80° C. for 3.5 h. Then triethyl orthoformate (384.35 mg, 431.86 μL, 2.59 mmol, 1.200 eq) was added and the stirring continued at 80° C. for 1 h 30 min, then at 50° C. overnight. The reaction mixture was allowed to cool to ambient temperature and was then concentrated under reduced pressure. The residue was dissolved in ethyl acetate and washed with sat. aq. NaHCO₃ solution and brine. The aq. layers were back-extracted with ethyl acetate. The combined organic layers were dried over magnesium sulfate, filtered and concentrated. The residue was suspended in TBME, filtered, and the solid collected. This solid was suspended in dichloromethane, filtered, and the solid collected to afford the title compound (143 mg, 19% yield). The mother liquor was concentrated and the residue purified by silica gel chromatography using ethyl acetate:heptane as eluent to afford another batch of title compound (66 mg, 9% yield). MS: 348.1 [M+H]⁺, ESI pos.

Both batches were combined used as such in subsequent chemistry.

### Intermediate 4

### 5-amino-1-[2-fluoro-4-(fluoromethoxy)phenyl]imidazole-4-carboxylic acid ethyl ester

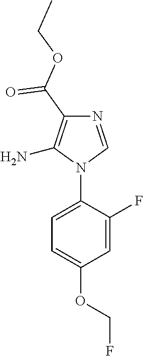 [SMILES: CCOC(=O)c1ncn(-c2ccc(OCF)cc2F)c1N]

### Step 1:2-fluoro-4-(fluoromethoxy)-1-nitrobenzene

To a solution of 3-fluoro-4-nitrophenol (2.3 g, 15 mmol, 1.0 eq) in acetonitrile (13 mL) were added DIEA (3.78 g, 5.11 mL, 29.3 mmol, 2.00 eq) and fluoro (iodo) methane (4.93 g, 2.09 mL, 29.3 mmol, 2.00 eq). The vessel was sealed and the mixture was stirred at ambient temperature overnight. The reaction mixture was directly concentrated under reduced pressure, the residue was suspended in ethyl acetate:heptane 1:1, the solid was filtered and discarded. The mother liquor was purified by silica gel chromatography using heptane/ethyl acetate as eluent to afford the title compound (2.68 g, 95% yield).

¹H NMR (300 MHz, CHLOROFORM-d) δ=8.22-8.08 (m, 1H), 7.02-6.92 (m, 2H), 5.78 (d, J=52.8 Hz, 2H)

### Step 2: [2-fluoro-4-(fluoromethoxy)phenyl]amine

2-fluoro-4-(fluoromethoxy)-1-nitrobenzene (2.68 g, 14.2 mmol, 1.00 eq) was dissolved in methanol (50 mL) and 10% palladium on carbon (754.03 mg, 708.54 μmol, 0.050 eq) was added. The reaction vessel was evacuated and back-filled with hydrogen gas 3 times. Then, the reaction was stirred at ambient temperature under hydrogen atmosphere for 2 hours. The reaction mixture was filtered over dicalite and the obtained solution concentrated under reduced pressure to afford the title compound (2.08 g, 89% yield).

¹H NMR (300 MHz, CHLOROFORM-d) δ=6.81 (dd, J=2.0, 11.5 Hz, 1H), 6.77-6.68 (m, 2H), 5.60 (d, J=55.0 Hz, 2H), 3.57 (br s, 2H)

### Step 3:5-amino-1-[2-fluoro-4-(fluoromethoxy)phenyl]imidazole-4-carboxylic acid ethyl ester

To a solution of 2-amino-2-cyano-acetic acid ethyl ester (intermediate 1, 2.41 g, 15.1 mmol, 1.20 eq) in acetonitrile (10 mL) was added a solution of [2-fluoro-4-(fluoromethoxy)phenyl]amine (2.08 g, 12.6 mmol, 1.00 eq) in acetonitrile (10 mL). The reaction mixture was stirred at 80° C. for 3 h. Then, triethyl orthoformate (2.28 g, 2.56 mL, 15.1 mmol, 1.20 eq) was added and the reaction mixture was stirred at 80° C. for another 90 min. The reaction mixture was allowed to cool to ambient temperature and was then concentrated under reduced pressure. The residue was dissolved in ethyl acetate and washed with sat. aq. NaHCO₃ solution and brine. The aqueous layers were back-extracted with ethyl acetate. The combined organic layers were dried over magnesium sulfate, filtered and concentrated under reduced pressure. The residue was suspended in TBME and the resulting solid filtered, collected, and dried to afford the title compound (2.23 g, 58% yield). MS: 298.1 [M+H]⁺, ESI pos.

### Intermediate 5

### 5-(tert-butoxycarbonylamino)-1-[2,3-difluoro-4-(fluoromethoxy)phenyl]imidazole-4-carboxylic acid

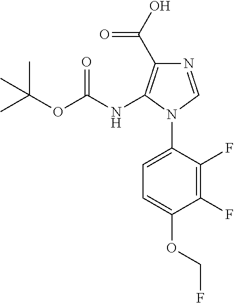 [SMILES: CC(C)(C)OC(=O)Nc1c(C(=O)O)ncn1-c1ccc(OCF)c(F)c1F]

### Step 1: ethyl 5-[bis(tert-butoxycarbonyl)amino]-1-[2,3-difluoro-4-(fluoromethoxy)phenyl]imidazole-4-carboxylate

To a solution of ethyl 5-amino-1-[2,3-difluoro-4-(fluoromethoxy)phenyl]imidazole-4-carboxylate (intermediate 2, 4.5 g, 14 mmol, 1.0 eq), DMAP (0.88 g, 7.1 mmol, 0.5 eq), triethylamine (7.96 mL, 57.1 mmol, 4.0 eq) in DCM (60 mL) was added di-t-butyldicarbonate (12.46 g, 57.1 mmol, 4.0 eq) dropwise at 0° C. under N₂, then the mixture was stirred at 25° C. for 16 h. Then, the reaction mixture was cooled to room temperature, ethyl acetate (100 mL) and water (100 mL) were added to the above mixture and the layers were separated. The aqueous phase was extracted with ethyl acetate (2×100 mL). Combined extracts were washed with brine (50 mL), dried over Na₂SO₄, filtered, and the filtrate was concentrated under reduced pressure. The residue was purified by silica gel column chromatography (petroleum ether:ethyl acetate) to afford the title compound (5.0 g, 9.7 mmol, 68% yield). MS: m/z=516.2 [M+H]⁺, ESI pos

### Step 2: 5-(tert-butoxycarbonylamino)-1-[2,3-difluoro-4-(fluoromethoxy)phenyl]imidazole-4-carboxylic acid

To a solution of methyl 5-[bis(tert-butoxycarbonyl)amino]-1-[2,3-difluoro-4-(fluoromethoxy)phenyl]imidazole-4-carboxylate (1.5 g, 3.0 mmol, 1.0 eq) in water (6 mL) and THF (6 mL) was added LiOH·H₂O (627.57 mg, 14.96 mmol, 5.0 eq) the mixture was stirred at 20° C. for 2 h. Then, ethyl acetate (30 mL) and water (30 mL) were added to the mixture and the layers were separated. The aqueous phase was extracted with ethyl acetate (30 mL), the combined organic layers were discarded. The aqueous was adjusted to pH=7 using 10% aqueous citric acid solution, and extracted with ethyl acetate (3×40 mL). The combined extracts were washed with brine (50 mL), dried over Na₂SO₄, filtered, and the filtrate was concentrated under reduced pressure. The residue was diluted with water (50 ml) followed by lyophilization to afford the title compound (1.11 g, 2.87 mmol, 90% yield). MS: m/z=388.0 [M+H]⁺, ESI pos.

The following compounds were prepared in analogy to the procedure above

| Int | Structure | Name | Reagents | MS |
|---|---|---|---|---|
| 6 | 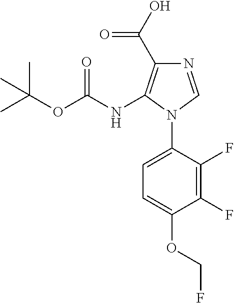 | 5-(tert-butoxycarbonylamino)- 1-[2-(difluoromethyl)-3- fluoro-4-(fluoromethoxy) phenyl]imidazole-4- carboxylic acid | Intermediate 3, DIPEA in step 1 instead of Et3N, 40° C. in step 2 | m/z = 420.1 [M + H]+, ESI pos |
| 7 | 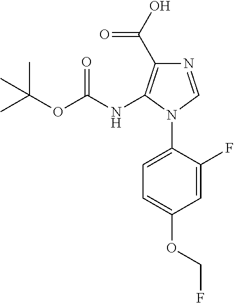 [SMILES: CC(C)(C)OC(=O)Nc1c(C(=O)O)ncn1-c1ccc(OCF)cc1F] | 5-(tert-butoxycarbonylamino)- 1-[4-(difluoromethoxy)-2,3- difluoro-phenyl]imidazole-4- carboxylic acid | Intermediate 4, step 1 at 30° C., step 2 at 30° C. | m/z = MS: 370.2 [M + H]+, ESI pos. |

### Intermediate 8

### 5-(tert-butoxycarbonylamino)-1-[2-fluoro-4-(fluoromethoxy)-3-(trifluoromethyl)phenyl]imidazole-4-carboxylic acid

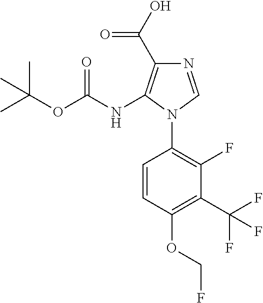 [SMILES: CC(C)(C)OC(=O)Nc1c(C(=O)O)ncn1-c1ccc(OCF)c(C(F)(F)F)c1F]

### Step 1:5-amino-1-[4-bromo-2-fluoro-3-(trifluoromethyl)phenyl]imidazole-4-carboxylic acid ethyl ester

To a solution of 2-amino-2-cyano-acetic acid ethyl ester (intermediate 1, 1.99 g, 11.6 mmol, 1.20 eq) in acetonitrile (12.5 mL) was added solution of [4-bromo-2-fluoro-3-(trifluoromethyl)phenyl]amine (2.5 g, 9.7 mmol, 1.0 eq) in acetonitrile (12.5 mL). The reaction mixture was stirred at 80° C. for 3 h. Then triethyl orthoformate (1.76 g, 1.98 mL, 11.6 mmol, 1.20 eq) was added and the reaction mixture was stirred another 3 hours at 80° C. The reaction mixture was allowed to cool to ambient temperature and was stored in the refrigerator overnight. The reaction was concentrated under reduced pressure. The residue was dissolved in ethyl acetate and washed with saturated aqueous NaHCO₃ solution and brine. The aqueous layers were back-extracted with ethyl acetate. The combined organic layers were dried over magnesium sulfate, filtered and partly concentrated. The resulting suspension was filtered and the solid collected to afford the title compound (1.2 g, 30% yield).

The mother liquor was concentrated under reduced pressure, the residue was diluted with dichloromethane and the resulting suspension filtered. The obtained solution was purified by silica gel chromatography using heptane/ethyl acetate as eluent to afford more title compound with a purity of approximately 80-85% (465 mg, 10% yield). The two batches were combined and used without further purification. LC-MS: 398.1 [M+H]⁺, ESI pos, bromine isotopic pattern.

### Step 2:5-[bis(tert-butoxycarbonyl)amino]-1-[4-bromo-2-fluoro-3-(trifluoromethyl)phenyl]imidazole-4-carboxylic acid ethyl ester

To a suspension of 5-amino-1-[4-bromo-2-fluoro-3-(trifluoromethyl)phenyl]imidazole-4-carboxylic acid ethyl ester (1.44 g, 3.46 mmol, 1.00 eq) and DIEA (1.34 g, 1.81 mL, 10.4 mmol, 3.00 eq) in dichloromethane (20.03 mL) was added (Boc)₂O (1.89 g, 2.01 mL, 8.65 mmol, 2.50 eq) and 4-dimethylaminopyridine (84.5 mg, 692 μmol, 0.200 eq) and the reaction was stirred 15 h at ambient temperature. The solvent was evaporated. The residue was dissolved in ethyl acetate and extracted with water. The aqueous layer was extracted 2 additional times with ethyl acetate. The combined organic layers were washed with brine, dried over magnesium sulfate, filtered and concentrated. The residue was suspended in ethyl acetate/heptane 1:1 and stirred for 1 h. Filtration afforded the title compound (1.6 g, 78% yield). MS: 598.4 [M+H]⁺, ESI pos bromine isotopic pattern.

### Step 3: 5-[bis(tert-butoxycarbonyl)amino]-1-[2-fluoro-4-(4,4,5,5-tetramethyl-1,3,2-dioxaborolan-2-yl)-3-(trifluoromethyl)phenyl]imidazole-4-carboxylic acid ethyl ester

To a mixture of 5-[bis(tert-butoxycarbonyl)amino]-1-[4-bromo-2-fluoro-3-(trifluoromethyl)phenyl]imidazole-4-carboxylic acid ethyl ester (1000 mg, 1.68 mmol, 1.000 eq), potassium acetate (493.69 mg, 5.03 mmol, 3.000 eq) and bis(pinacolato)diboron (510.97 mg, 2.01 mmol, 1.200 eq) was added dry 1,2-dimethoxyethane (9.52 g, 11.0 mL, 105.6 mmol, 63.00 eq) and the mixture was flushed with argon. 1,1′-bis(Diphenylphosphino)ferrocene palladium(ii)chloride dichloromethane complex (150.63 mg, 184.45 μmol, 0.110 eq) was added in one portion and the reaction vessel sealed. The mixture was then heated to 90° C. for 12 h. The reaction was cooled to room temperature, diluted with water, and extracted 3 times with ethyl acetate. The combined organic layers were dried over magnesium sulfate, filtered and concentrated under reduced pressure. The residue was purified by silica gel chromatography using 9:1 dichloromethane:MeOH+0.25% NH₃) as eluent to afford the title compound (452 mg, 38% yield). MS: 644.4 [M+H]⁺, ESI pos.

### Step 4: 5-[bis(tert-butoxycarbonyl)amino]-1-[2-fluoro-4-hydroxy-3-(trifluoromethyl)phenyl]imidazole-4-carboxylic acid ethyl ester

5-[bis(tert-butoxycarbonyl)amino]-1-[2-fluoro-4-(4,4,5,5-tetramethyl-1,3,2-dioxaborolan-2-yl)-3-(trifluoromethyl)phenyl]imidazole-4-carboxylic acid ethyl ester (452 mg, 632 μmol, 1.00 eq) was dissolved in tetrahydrofuran (12 mL) and water (4.5 mL). Then, sodium perborate tetrahydrate (389.1 mg, 2.53 mmol, 4.000 eq) was added in 4 portions and the reaction was stirred 16 hours at room temperature. The reaction was diluted with water and extracted 3 times with ethyl acetate. The combined ethyl acetate layers were washed with brine dried over magnesium sulfate, filtered and concentrated to afford the title compound (362 mg, quant). MS: 534.4 [M+H]⁺, ESI pos.

### Step 5: 5-[bis(tert-butoxycarbonyl)amino]-1-[2-fluoro-4-(fluoromethoxy)-3-(trifluoromethyl)phenyl]imidazole-4-carboxylic acid ethyl ester

To a solution of 5-[bis(tert-butoxycarbonyl)amino]-1-[2-fluoro-4-hydroxy-3-(trifluoromethyl)phenyl]imidazole-4-carboxylic acid ethyl ester (500 mg, 909 μmol, 1.000 eq) and DIEA (352.49 mg, 476.34 μL, 2.73 mmol, 3.000 eq) in acetonitrile (12 mL) was added fluoro (iodo) methane (290.8 mg, 123.2 μL, 1.82 mmol, 2.000 eq). The tube was sealed and heated to 50° C. for 6 hours. The reaction was allowed to stand at room temperature overnight. The solvent was evaporated and the residue diluted with ethyl acetate and water. The aqueous layer was extracted 2 additional times with ethyl acetate. The combined organic layers were washed with brine, dried over magnesium sulfate and concentrated under reduced pressure. The residue was purified by silica gel chromatography using ethyl acetate/heptane as eluent to afford the title compound (446 mg; 87% yield). MS: 566.4 [M+H]⁺, ESI pos.

### Step 6: 5-(tert-butoxycarbonylamino)-1-[2-fluoro-4-(fluoromethoxy)-3-(trifluoromethyl)phenyl]imidazole-4-carboxylic acid

To a mixture of 5-[bis(tert-butoxycarbonyl)amino]-1-[2-fluoro-4-(fluoromethoxy)-3-(trifluoromethyl)phenyl]imidazole-4-carboxylic acid ethyl ester (440 mg, 778 μmol, 1.00 eq) in tetrahydrofuran (7 mL) and water (3 mL) was added 1 M lithium hydroxide solution in water (3.89 mL, 3.89 mmol, 5.00 eq) and lithium hydroxide monohydrate (326.51 mg, 7.78 mmol, 10.000 eq). The reaction was heated to 30° C. for 15 h. The reaction was cooled to room temperature. Then, 25% aqueous HCl (1.89 mL, 15.6 mmol, 20.0 eq) was slowly added (pH between 1-2). The mixture was extracted with ethyl acetate (3 times). The combined organic layers were washed with brine, dried over MgSO₄, filtered and evaporated to afford the title compound (350 mg, 100%). MS: 438.3 [M+H]⁺, ESI pos.

### Intermediate 9

### 5-(tert-butoxycarbonylamino)-1-[4-(difluoromethoxy)-2,3-difluoro-phenyl]imidazole-4-carboxylic acid

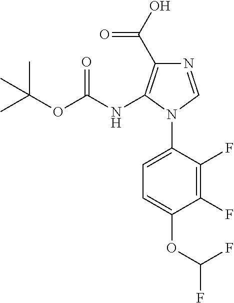 [SMILES: CC(C)(C)OC(=O)Nc1c(C(=O)O)ncn1-c1ccc(OC(F)F)c(F)c1F]

### Step 1: 1-(difluoromethoxy)-2,3-difluoro-4-nitrobenzene

To a mixture of 2,3-difluoro-4-nitrophenol (1 g, 5.7 mmol, 1.0 eq) in acetonitrile (6 mL) were added DIEA (1.48 g, 1.99 mL, 11.4 mmol, 2.00 eq) and a 2.5 M difluoro (iodo) methane solution in acetonitrile (4.57 mL, 11.4 mmol, 2.00 eq). The reaction vessel was sealed and the mixture was stirred at ambient temperature over the weekend. The reaction mixture was directly evaporated and the residue was extracted using ethyl acetate and deion. H₂O. The organic layer was washed with brine, dried over MgSO₄, filtered and concentrated under reduced pressure. The residue was purified by silica gel chromatography using ethyl acetate:heptane as eluent to afford the title compound (575 mg, 44% yield).

¹H NMR (300 MHz, CHLOROFORM-d) δ=7.94 (ddd, J=2.4, 7.4, 9.6 Hz, 1H), 7.21 (tddd, J=1.0, 2.2, 6.9, 9.3 Hz, 1H), 6.69 (t, J=71.3 Hz, 1H)

### Step 2: [4-(difluoromethoxy)-2,3-difluoro-phenyl]amine

1-(difluoromethoxy)-2,3-difluoro-4-nitro-benzene (575 mg, 2.5 mmol, 1.000 eq) was dissolved in methanol (5 mL) and 10% palladium on carbon (266.4 mg, 250.3 μmol, 0.100 eq) was added. The suspension was evacuated 3 times and back-filled with hydrogen and the reaction stirred 2.5 h at ambient temperature. The reaction mixture was filtered over dicalite and evaporated to afford the title compound (455 mg, 91% yield). MS: 196.0 [M+H]⁺, ESI pos.

### Step 3: 5-amino-1-[4-(difluoromethoxy)-2,3-difluoro-phenyl]imidazole-4-carboxylic acid ethyl ester

To a solution of 2-amino-2-cyanoacetic acid ethyl ester (intermediate 1, 247.13 mg, 1.93 mmol, 1.500 eq) in acetonitrile (4 mL) was added a solution of [4-(difluoromethoxy)-2,3-difluorophenyl]amine (256 mg, 1.29 mmol, 1.00 eq) in acetonitrile (4 mL). The reaction mixture was stirred at 80° C. for 3 h. Then triethyl orthoformate (285.84 mg, 321.17 μL, 1.93 mmol, 1.500 eq) was added and the reaction mixture was stirred at 80° C. for 1 hour 45 minutes. The reaction mixture was allowed to cool to ambient temperature and was stored in the refrigerator overnight. The reaction was concentrated under reduced pressure. The residue was dissolved in ethyl acetate and washed with saturated aqueous NaHCO₃ solution and brine. The aqueous layers were back-extracted with ethyl acetate. The combined organic layers were dried over magnesium sulfate, filtered and concentrated under reduced pressure. The residue was suspended in TBME, filtered, and the solid collected to afford the title compound (162 mg, 37% yield). MS: 334.0 [M+H]⁺, ESI pos.

### Step 4:5-[bis(tert-butoxycarbonyl)amino]-1-[4-(difluoromethoxy)-2,3-difluorophenyl]imidazole-4-carboxylic acid ethyl ester

To a light suspension of 5-amino-1-[4-(difluoromethoxy)-2,3-difluorophenyl]imidazole-4-carboxylic acid ethyl ester (156 mg, 454 μmol, 1.00 eq) and DIEA (176.06 mg, 237.92 μL, 1.36 mmol, 3.000 eq) in dichloromethane (4 mL) was added (Boc)₂O (247.76 mg, 263.58 μL, 1.14 mmol, 2.500 eq) and 4-dimethylaminopyridine (11.1 mg, 90.8 μmol, 0.200 eq). The reaction was stirred 80 min at ambient temperature. Then, the reaction was diluted with water and extracted 3 times with dichloromethane. The combined organic layers were washed with brine, dried over magnesium sulfate, filtered and concentrated under reduced pressure. The residue was purified by silica gel chromatography using heptane/ethyl acetate as eluent to afford the title compound (226 mg, 91% yield). MS: 534.1 [M+H]⁺, ESI pos.

### Step 5: 5-(tert-butoxycarbonylamino)-1-[4-(difluoromethoxy)-2,3-difluorophenyl]imidazole-4-carboxylic acid

To a mixture of 5-[bis(tert-butoxycarbonyl)amino]-1-[4-(difluoromethoxy)-2,3-difluorophenyl]imidazole-4-carboxylic acid ethyl ester (226 mg, 415 μmol, 1.00 eq) in tetrahydrofuran (4 mL) and water (1.5 mL) was added 1 M LiOH in water (2.08 mL, 2.08 mmol, 5.00 eq) and LiOH (99.43 mg, 4.15 mmol, 10.00 eq). The reaction was heated to 40° C. for 15 h. The reaction was cooled to room temperature. Then, 25% aq. HCl (858 μL, 7.06 mmol, 17.0 eq) was slowly added (pH between 1 and 2). The mixture was extracted with ethyl acetate (3 times). The combined organic layers were washed with brine, dried over MgSO₄, filtered and evaporated to afford the title compound (178 mg, 99% yield). MS: 406.1 [M+H]⁺, ESI pos.

### Intermediate 10

### 4-[[5-(tert-butoxycarbonylamino)-1-[2,3-difluoro-4-(fluoromethoxy)phenyl]imidazole-4-carbonyl]amino]-2-chlorobenzoic acid

### Step 1: methyl 4-[[5-(tert-butoxycarbonylamino)-1-[2,3-difluoro-4-(fluoromethoxy)phenyl]imidazole-4-carbonyl]amino]-2-chlorobenzoate

To a stirred mixture of 5-[(tert-butoxycarbonyl)amino]-1-[2,3-difluoro-4-(fluoromethoxy)phenyl]imidazole-4-carboxylic acid (intermediate 5, 28 g, 72 mmol, 1 equiv) and 1-methylimidazole (29.68 g, 361.5 mmol, 5 equiv) in ACN (280 mL) was added chloro-N,N,N′,N′-tetramethylformamidinium hexafluorophosphate (40.57 g, 144.6 mmol, 2 equiv) at room temperature under nitrogen atmosphere. The resulting mixture was stirred for 1 h at 30° C. To the above mixture was added methyl 4-amino-2-chlorobenzoate ([46004-37-9], 26.84 g, 144.59 mmol, 2 equiv) at room temperature. The resulting mixture was stirred for additional 3 h at 30° C. The resulting mixture was diluted with brine (300 mL), extracted with EtOAc (3×200 mL), the combined organic layers were washed with brine (3×200 mL), and dried over anhydrous Na₂SO₄. After filtration, the filtrate was concentrated under reduced pressure. The residue was purified by silica gel column chromatography using PE/EA as eluent to afford the title compound (15 g, 37% yield). MS: m/z=553.0 [M−H], ESI neg.

### Step 2: 4-[[5-(tert-butoxycarbonylamino)-1-[2,3-difluoro-4-(fluoromethoxy)phenyl]imidazole-4-carbonyl]amino]-2-chlorobenzoic acid

To a stirred mixture of methyl 4-[[5-(tert-butoxycarbonylamino)-1-[2,3-difluoro-4-(fluoromethoxy)phenyl]imidazole-4-carbonyl]amino]-2-chlorobenzoate (10.0 g, 18.0 mmol, 1.0 eq) in THF (100 mL) was added LiOH·H₂O (2.27 g, 54.1 mmol, 3.0 eq) in water (20 mL) dropwise at room temperature. The resulting mixture was stirred for 16 h at 30° C. The resulting mixture was concentrated under reduced pressure. The residue was diluted with water (100 mL) and the mixture was acidified to pH 6 with 1 M HCl (125 mL). The precipitated solids were collected by filtration and washed with water (3×50 mL) to afford the title compound (6.51 g, 12.0 mmol, 65.4% yield). MS: m/z=541.15 [M+H]⁺, ESI pos.

### Intermediate 11

### N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester

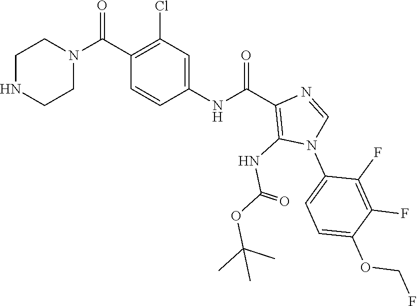 [SMILES: CC(C)(C)OC(=O)Nc1c(C(=O)Nc2ccc(C(=O)N3CCNCC3)c(Cl)c2)ncn1-c1ccc(OCF)c(F)c1F]

Under argon atmosphere 4-[[5-(tert-butoxycarbonylamino)-1-[2,3-difluoro-4-(fluoromethoxy)phenyl]imidazole-4-carbonyl]amino]-2-chlorobenzoic acid (intermediate 10, 1000 mg, 1.85 mmol, 1.000 eq) was dissolved in N,N-dimethylformamide, extra dry (20 mL), then DIEA (716.83 mg, 968.69 μL, 5.55 mmol, 3.000 eq) was added, followed by 1-piperazinecarboxylic acid, 9H-fluoren-9-ylmethyl ester, hydrochloride (715.62 mg, 2.03 mmol, 1.100 eq) and HATU (359.98 mg, 946.73 μmol, 1.100 eq). The reaction was stirred at ambient temperature for 20 h. Then, the reaction mixture was concentrated under reduced pressure and the residue extracted using ethyl acetate and water. The organic layer was washed with brine, dried over MgSO₄, filtered and then concentrated under reduced pressure. To a solution of the residue in tetrahydrofuran, extra dry (10 mL) was added 2 M dimethylamine solution in THF (2.88 g, 3.24 mL, 6.47 mmol, 3.50 eq) at ambient temperature and the reaction stirred for 18 hours. The solvent was evaporated and the residue was purified by silica gel chromatography using dichloromethane/(dichloromethane:MeOH=9:1+0.25% NH₃) as eluent to afford the title compound (1.08 g, 91% yield). MS: m/z=609.2 [M+H]⁺, ESI pos.

### Intermediate 12

### N-[3-[2,3-difluoro-4-(fluoromethoxy)phenyl]-5-[[3-fluoro-4-(piperazine-1-carbonyl)phenyl]carbamoyl]imidazol-4-yl]carbamic acid tert-butyl ester;2,2,2-trifluoroacetic acid

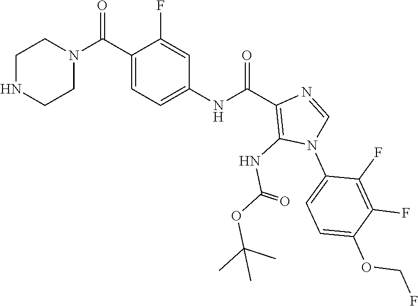 [SMILES: CC(C)(C)OC(=O)Nc1c(C(=O)Nc2ccc(C(=O)N3CCNCC3)c(F)c2)ncn1-c1ccc(OCF)c(F)c1F]

### Step 1: 4-(4-amino-2-fluorobenzoyl)piperazine-1-carboxylic acid 9H-fluoren-9-ylmethyl ester

To a mixture of 4-amino-2-fluorobenzoic acid (2 g, 12.6 mmol, 1.00 eq) in DMF, extra dry (20 mL) was added fmoc-piperazine hydrochloride (4.36 g, 12.6 mmol, 1.00 eq), DIPEA (4.9 g, 6.62 mL, 37.9 mmol, 3.00 eq) and HATU (7.21 g, 19.0 mmol, 1.50 eq). The clear brown solution stirred at ambient temperature for 15 h. The reaction mixture was concentrated, the residue diluted with water and extracted 3 times with ethyl acetate. The combined organic layers were washed with aq. NaHCO₃-solution, water and brine, dried over magnesium sulfate, filtered and concentrated. The residue was adsorbed on isolute HM-N and purified by silica gel chromatography using dichloromethane/(dichloromethane/methanol+0.25% NH₄OH) as eluent. Clean fractions were combined and concentrated under reduced pressure. The residue dissolved in ethyl acetate stirred at room temperature overnight. Filtration of the resulting suspension and collection of the solid afforded the title compound (2.87 g; 48% yield).

The mother liquor was combined with mixed fractions of the previous chromatography, adsorbed onto isolute HM-N, and was purified again by silica gel chromatography using heptane/ethyl acetate as eluent to afford more title compound with a purity of 85% (1.37 g; 22% yield). MS: m/z=446.3 [M+H]⁺, ESI pos.

### Step 2:4-[4-[[5-(tert-butoxycarbonylamino)-1-[2,3-difluoro-4-(fluoromethoxy)phenyl]imidazole-4-carbonyl]amino]-2-fluorobenzoyl]piperazine-1-carboxylic acid 9H-fluoren-9-ylmethyl ester

To a suspension of 5-(tert-butoxycarbonylamino)-1-[2,3-difluoro-4-(fluoromethoxy)phenyl]imidazole-4-carboxylic acid (intermediate 5, 200 mg, 506 μmol, 1.00 eq) and 4-(4-amino-2-fluorobenzoyl)piperazine-1-carboxylic acid 9H-fluoren-9-ylmethyl ester (261.04 mg, 556.66 μmol, 1.100 eq) in dichloromethane (7 mL) was added 1-methylimidazole (124.64 mg, 121.01 μL, 1.52 mmol, 3.000 eq). The reaction mixture was cooled to 0° C. after which n-propylphosphonic acid anhydride, cyclic trimer 50% in EtOAc (644.07 mg, 596.36 μL, 1.01 mmol, 2.000 eq) was added and stirred for 1 h at 0° C. Then, the reaction was heated to 40° C. for 19 h. The reaction was cooled down to room temperature and then concentrated under reduced pressure. The residue was dissolved in ethyl acetate and water and the mixture extracted with ethyl acetate. The combined organic layers were washed with brine, dried over MgSO₄, filtered and concentrated under reduced pressure. The residue was purified by reverse phase chromatography (Phenomenex Gemini-NX 5u 110A, I: 100 mm, dia: 30 mm) using acetonitrile/water (+0.05% HCl) as eluent. The corresponding fractions were combined and lyophilized to afford the title compound (292 mg, 71% yield). MS: m/z=815.2 [M+H]⁺, ESI pos.

### Step 3: N-[3-[2,3-difluoro-4-(fluoromethoxy)phenyl]-5-[[3-fluoro-4-(piperazine-1-carbonyl)phenyl]carbamoyl]imidazol-4-yl]carbamic acid tert-butyl ester

To a solution of 4-[4-[[5-(tert-butoxycarbonylamino)-1-[2,3-difluoro-4-(fluoromethoxy)phenyl]imidazole-4-carbonyl]amino]-2-fluorobenzoyl]piperazine-1-carboxylic acid 9H-fluoren-9-ylmethyl ester (292 mg, 0.358 mmol, 1.00 eq) in tetrahydrofuran (3 mL) was added 2M dimethylamine solution in THF (558.18 mg, 627.16 μL, 1.25 mmol, 3.500 eq) and the reaction stirred 18 hours at ambient temperature. The solvent was evaporated and the residue purified by silica gel chromatography using CH₂Cl₂/(CH₂Cl₂:MeOH=9:1+0.25% NH₃) as eluent to afford the title compound (208 mg, 98% yield). MS: 593.2 [M+H]⁺, ESI pos.

The following compounds were prepared in analogy to the procedure above

| Int | Structure | Name | Reagents | MS |
|---|---|---|---|---|
| 13 | 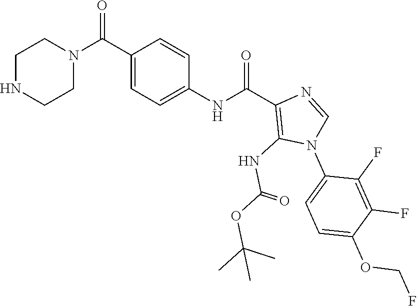 [SMILES: CC(C)(C)OC(=O)Nc1c(C(=O)Nc2ccc(C(=O)N3CCNCC3)cc2)ncn1-c1ccc(OCF)c(F)c1F] | N-[3-[2,3-difluoro- 4-(fluoromethoxy) phenyl]-5-[4- (piperazine-1- carbonyl)phenyl] carbamoyl]imidazol- 4-yl]carbamic acid tert-butyl ester | 4- aminobenzoic acid in step 1 | m/z = 575.3 [M + H+]+, ESI pos |
| 14 | 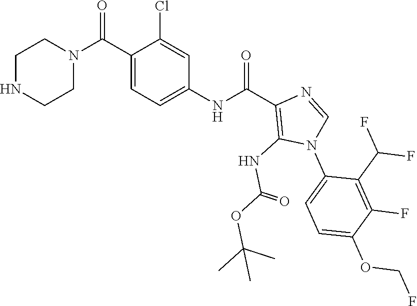 [SMILES: CC(C)(C)OC(=O)Nc1c(C(=O)Nc2ccc(C(=O)N3CCNCC3)c(Cl)c2)ncn1-c1ccc(OCF)c(F)c1C(F)F] | N-[5-[[3-chloro-4- (piperazine-1- carbonyl)phenyl] carbamoyl]-3-[2- (difluoromethyl)-3- fluoro-4- (fluoromethoxy) phenyl]imidazol-4- yl]carbamic acid tert-butyl ester | 4-amino-2- chlorobenzoic acid in step 1, intermediate 6 in step 2 | m/z = 639.2 [M − H]−, ESI neg |
| 15 | 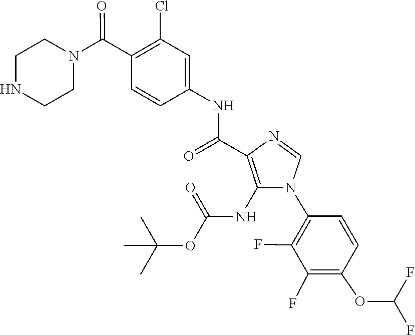 [SMILES: CC(C)(C)OC(=O)Nc1c(C(=O)Nc2ccc(C(=O)N3CCNCC3)c(Cl)c2)ncn1-c1ccc(OC(F)F)c(F)c1F] | N-[5-[[3-chloro-4- (piperazine-1- carbonyl)phenyl] carbamoyl]-3-[4- (difluoromethoxy)- 2,3-difluoro- phenyl]imidazol-4- yl]carbamic acid tert-butyl ester | 4-amino-2- chlorobenzoic acid in step 1, intermediate 9 in step 2 | m/z = 627.2 [M + H]+, ESI pos |
| 16 | 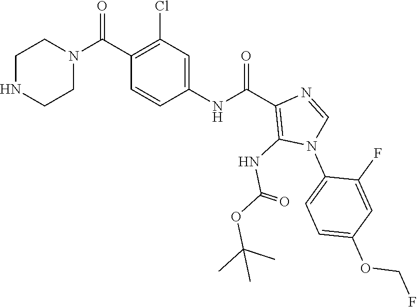 [SMILES: CC(C)(C)OC(=O)Nc1c(C(=O)Nc2ccc(C(=O)N3CCNCC3)c(Cl)c2)ncn1-c1ccc(OCF)cc1F] | N-[5-[[3-chloro-4- (piperazine-1- carbonyl)phenyl] carbamoyl]-3- [2-fluoro-4- (fluoromethoxy) phenyl]imidazol-4- yl]carbamic acid tert-butyl ester | 4-amino-2- chlorobenzoic acid in step 1, intermediate 7 in step 2 | m/z = 589.3 [M − H]−, ESI neg |

### Intermediate 17

### N-[5-[[4-(azetidin-3-ylcarbamoyl)-3-chloro-phenyl]carbamoyl]-3-[2,3-difluoro-4-(fluoromethoxy)phenyl]imidazol-4-yl]carbamic acid tert-butyl ester

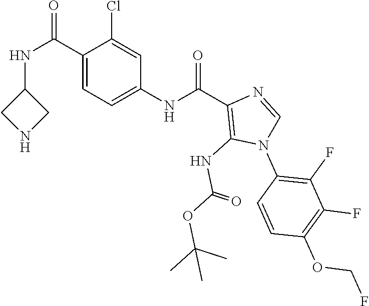 [SMILES: CC(C)(C)OC(=O)Nc1c(C(=O)Nc2ccc(C(=O)NC3CNC3)c(Cl)c2)ncn1-c1ccc(OCF)c(F)c1F]

### Step 1: 3-[[4-[[5-(tert-butoxycarbonylamino)-1-[2,3-difluoro-4-(fluoromethoxy)phenyl]imidazole-4-carbonyl]amino]-2-chlorobenzoyl]amino]azetidine-1-carboxylic acid benzyl ester

Under argon atmosphere 4-[[5-(tert-butoxycarbonylamino)-1-[2,3-difluoro-4-(fluoromethoxy)phenyl]imidazole-4-carbonyl]amino]-2-chlorobenzoic acid (intermediate 10, 250 mg, 462 μmol, 1.00 eq) was dissolved in N,N-dimethylformamide (5 mL), then DIEA (238.94 mg, 322.9 L, 1.85 mmol, 4.000 eq) was added, followed by HATU (193.32 mg, 508.43 μmol, 1.100 eq) and 3-aminoazetidine-1-carboxylic acid benzyl ester (104.86 mg, 508.43 μmol, 1.100 eq). The reaction was stirred at ambient temperature overnight. The mixture was directly concentrated under reduced pressure and the residue purified by preparative reverse phase HPLC (column: Phenomenex Gemini-NX 5u 110A, I: 100 mm, dia 30 mm) using water containing 0.05% TFA/acetonitrile as eluent. The corresponding fractions were combined to afford the title compound (332 mg, 96% yield). MS: m/z=729.6 [M+H]⁺, ESI pos.

### Step 2: N-[5-[[4-(azetidin-3-ylcarbamoyl)-3-chloro-phenyl]carbamoyl]-3-[2,3-difluoro-4-(fluoromethoxy)phenyl]imidazol-4-yl]carbamic acid tert-butyl ester

3-[[4-[[5-(tert-butoxycarbonylamino)-1-[2,3-difluoro-4-(fluoromethoxy)phenyl]imidazole-4-carbonyl]amino]-2-chloro-benzoyl]amino]azetidine-1-carboxylic acid benzyl ester (306 mg, 411 μmol, 1.00 eq) was dissolved in methanol (4 mL) and 10% palladium on carbon (131.31 mg, 123.39 μmol, 0.300 eq) was added. The reaction was evacuated and backfilled with hydrogen 3 times. The mixture was stirred at ambient temperature for 15 h under hydrogen atmosphere. The reaction mixture was filtered, and the obtained solution concentrated under reduced pressure to afford the title compound, containing 10% de-chlorinated byproduct (227 mg, 83% yield). The material was used without further purification. MS: 595, 17 [M+H]⁺, ESI pos and 561.2 [M+H]⁺, ESI pos.

### Intermediate 18

### 4-[1-(4-amino-2-chloro-benzoyl) isonipecotoyl]piperazine-1-carboxylic acid tert-butyl ester

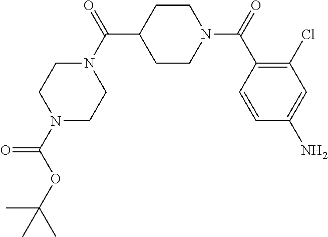 [SMILES: CC(C)(C)OC(=O)N1CCN(C(=O)C2CCN(C(=O)c3ccc(N)cc3Cl)CC2)CC1]

### Step 1: 1-(2-chloro-4-nitrobenzoyl) isonipecotic acid methyl ester

Under argon atmosphere 2-chloro-4-nitrobenzoic acid (1 g, 4.96 mmol, 1.000 eq) was dissolved in N,N-dimethylformamide, extra dry (20 mL), then DIEA (2.56 g, 3.47 mL, 19.8 mmol, 4.000 eq) was added, followed by methyl isonipecotate (781.4 mg, 737.2 μL, 5.46 mmol, 1.100 eq) and HATU (359.98 mg, 946.73 μmol, 1.100 eq). The reaction was stirred at ambient temperature for 1 h. Then the reaction mixture concentrated under reduced pressure and the residue was extracted using ethyl acetate and water. The organic layer was washed with brine, dried over MgSO₄, filtered and concentrated. The residue was purified by silica gel chromatography using heptane:EtOAc as eluent to afford the title compound (1.53 g, 93% yield). MS: m/z=327.0 [M+H]⁺, ESI pos.

### Step 2: 1-(2-chloro-4-nitrobenzoyl) isonipecotic acid

To a mixture of 1-(2-chloro-4-nitrobenzoyl) isonipecotic acid methyl ester (1.53 g, 4.68 mmol, 1.00 eq) in tetrahydrofuran (15 mL) was added 1 M LiOH solution in water (15 mL, 15 mmol, 3.2 eq) and LiOH·H₂O (2.36 g, 56.2 mmol, 12.0 eq). The reaction was stirred at ambient temperature for 45 min. Then, the mixture was acidified using HCl 25% (aq.)(9.11 mL, 74.9 mmol, 16.0 eq) and then extracted with ethyl acetate and water. The combined organic layers were washed with brine, dried over MgSO₄, filtered and concentrated under reduced pressure to afford the title compound (1.50 g, 99% yield). MS: m/z=313.0 [M+H]⁺ ESI pos.

### Step 3: 4-[1-(2-chloro-4-nitrobenzoyl) isonipecotoyl]piperazine-1-carboxylic acid tert-butyl ester

1-(2-chloro-4-nitro-benzoyl) isonipecotic acid (150 mg, 465 μmol, 1.00 eq) was dissolved in N, N-dimethylformamide (3 mL) under argon atmosphere, then DIEA (180.4 mg, 243.79 μL, 1.4 mmol, 3.000 eq) was added, followed by 1-Boc-piperazine (95.33 mg, 511.8 μmol, 1.100 eq) and HATU (194.61 mg, 511.82 μmol, 1.100 eq). The reaction was stirred at ambient temperature for 20 h. The reaction was poured onto water extracted with ethyl acetate. The combined organic layers were washed with brine, dried over MgSO₄, filtered and concentrated. The residue was purified by silica gel chromatography using heptane/ethyl acetate as eluent to afford the title compound (221 mg, 98% yield). MS: m/z=481.1 [M+H]⁺ ESI pos.

### Step 4: 4-[1-(4-amino-2-chlorobenzoyl) isonipecotoyl]piperazine-1-carboxylic acid tert-butyl ester

4-[1-(2-chloro-4-nitrobenzoyl) isonipecotoyl]piperazine-1-carboxylic acid tert-butyl ester (218 mg, 449 μmol, 1.00 eq) was dissolved in ethanol (4 mL) and water (1 mL), then ammonium chloride (600.09 mg, 11.22 mmol, 25.000 eq) and zinc (293.39 mg, 4.49 mmol, 10.000 eq) were added. The mixture was heated to 50° C. and stirred overnight. The reaction mixture was filtered and washed using ethyl acetate. The mother liquor was concentrated and the residue was extracted using ethyl acetate and water. The organic layer was washed with brine, dried with MgSO₄, filtered and concentrated. The residue was purified by silica gel chromatography using dichloromethane/(CH₂Cl₂:MeOH=9:1+0.25% NH₃) as eluent to afford the title compound (172 mg, 84% yield). MS: m/z=451.2 [M+H]⁺, ESI pos.

The following compounds were prepared in analogy to the procedure above

| Int | Structure | Name | Reagents | MS |
|---|---|---|---|---|
| 19 | 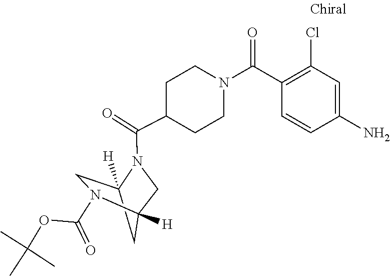 [SMILES: CC(C)(C)OC(=O)N1C[C@@H]2C[C@H]1CN2C(=O)C1CCN(C(=O)c2ccc(N)cc2Cl)CC1] | (1S,4S)-5-[1-(4-amino- 2-chloro-benzoyl) isonipecotoyl]-2,5- diazabicyclo[2.2.1] heptane-2- carboxylic acid tert-butyl ester | (1S,4S)-2,5- diazabicyclo [2.2.1] heptane-2- carboxylic acid tert-butyl ester in step 3 | m/z = 463.4 [M + H]+, ESI pos |
| 20 | 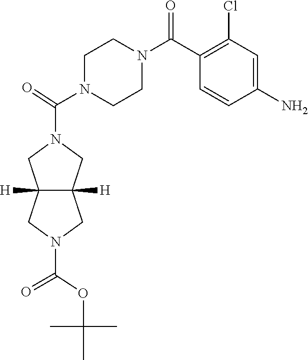 [SMILES: CC(C)(C)OC(=O)N1C[C@@H]2CN(C(=O)N3CCN(C(=O)c4ccc(N)cc4Cl)CC3)C[C@@H]2C1] | tert-butyl cis 2-[1-(4- amino-2- chlorobenzoyl) piperidine-4- carbonyl]- hexahydropyrrolo [3,4-c]pyrrole- 5-carboxylate | cis-2-boc- hexahydro- pyrrolo[3,4- c]pyrrole in step 3 | m/z = 477.2 [M + H]+, ESI pos |

### Intermediate 21

### (2-chloro-4-nitrophenyl)-piperazino-methanone

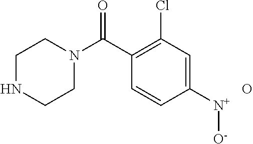

### Step 1: 4-(2-chloro-4-nitrobenzoyl)piperazine-1-carboxylic acid tert-butyl ester

Under argon atmosphere 2-chloro-4-nitrobenzoic acid (1 g, 5.0 mmol, 1.0 eq) was dissolved in N,N-dimethylformamide, extra dry (20 mL), then DIEA (2.56 g, 3.47 mL, 19.8 mmol, 4.00 eq) was added, followed by 1-Boc-piperazine (1.02 g, 5.46 mmol, 1.10 eq) and HATU (359.98 mg, 946.73 μmol, 1.100 eq). The reaction was stirred at ambient temperature for 30 min. Then the reaction mixture was concentrated under reduced pressure. The residue was extracted using ethyl acetate and water. The organic layer was washed with 1M NaOH, 2M Na₂CO₃ and brine, dried over MgSO₄, filtered and then concentrated under reduced pressure. The residue was purified by silica gel chromatography using heptane/EtOAc as eluent to afford the title compound (1.83 g, quant.). MS: m/z=314.1 [M+H-Buten]⁺, ESI pos.

### Step 2: (2-chloro-4-nitrophenyl)-piperazino-methanone

4-(2-chloro-4-nitrobenzoyl)piperazine-1-carboxylic acid tert-butyl ester (1.83 g, 4.95 mmol, 1.00 eq) was dissolved in dichloromethane (20 mL) and then TFA (5.64 g, 3.81 mL, 49.5 mmol, 10.0 eq) was added and the reaction stirred 1 hour at ambient temperature. The mixture was concentrated under reduced pressure and the residue was extracted with ethyl acetate and water. The organic layer was washed with 2M Na₂CO₃ and brine, dried over MgSO₄, filtered and evaporated to afford the title compound (1.16 g, 86% yield). MS: m/z=270.1 [M+H]⁺, ESI pos.

### Intermediate 22

### (1R,5S,6r)-6-[4-(4-amino-2-chlorobenzoyl)piperazine-1-carbonyl]-3-azabicyclo[3.1.0]hexane-3-carboxylic acid tert-butyl ester

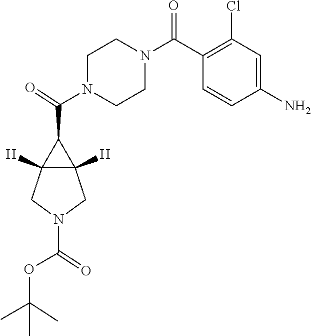 [SMILES: CC(C)(C)OC(=O)N1C[C@@H]2[C@H](C1)[C@H]2C(=O)N1CCN(C(=O)c2ccc(N)cc2Cl)CC1]

### Step 1: (1R,5S,6r)-6-[4-(2-chloro-4-nitrobenzoyl)piperazine-1-carbonyl]-3-azabicyclo[3.1.0]hexane-3-carboxylic acid tert-butyl ester

To a solution of (1R,5S,6r)-3-tert-butoxycarbonyl-3-azabicyclo[3.1.0]hexane-6-carboxylic acid (311.69 mg, 1.33 mmol, 1.200 eq) and DIEA (429.84 mg, 580.87 μL, 3.33 mmol, 3.00 eq) in N,N-dimethylformamide (3 mL) was added HATU (505.85 mg, 1.33 mmol, 1.20 eq). After 15 min a solution of (2-chloro-4-nitrophenyl)-piperazino-methanone (Intermediate 21, 302 mg, 1.11 mmol, 1.00 eq) in N,N-dimethylformamide (3 mL) was added and the mixture stirred 1 hour at room temperature. The solvent was evaporated. The residue was dissolved in ethyl acetate and washed with water, 2M Na₂CO₃ solution and brine. The aqueous layers were back-extracted with ethyl acetate 2 times. The combined organic layers were dried over magnesium sulfate, filtered and concentrated under reduced pressure. The residue was adsorbed on isolute HM-N and was purified by silica gel chromatography using heptane/ethyl acetate as eluent to afford the title compound (534 mg, 99% yield). MS: 523.3 [M+HCOO]⁻, ESI neg.

### Step 2: (1R,5S,6r)-6-[4-(4-amino-2-chlorobenzoyl)piperazine-1-carbonyl]-3-azabicyclo[3.1.0]hexane-3-carboxylic acid tert-butyl ester

(1R,5S,6r)-6-[4-(2-chloro-4-nitrobenzoyl)piperazine-1-carbonyl]-3-azabicyclo[3.1.0]hexane-3-carboxylic acid tert-butyl ester (519 mg, 1.06 mmol, 1.00 eq) was dissolved in ethanol (18 mL) and water (5 mL) and then ammonium chloride (1.14 g, 21.2 mmol, 20.0 eq) and iron (889.68 mg, 15.93 mmol, 15.00 eq) were added and stirred 1 h 15 min at ambient temperature. Then, the reaction was heated to 60° C. for 1.5 h. The mixture was filtered and then washed with ethyl acetate and the mother liquor was concentrated under reduced pressure. The residue was extracted using ethyl acetate and water. The organic layer was washed with brine, dried over MgSO₄, filtered and then concentrated to afford the title compound (502 mg; 100% yield). MS: 493.3 [M+HCOO]⁻, ESI neg.

### Intermediate 23

### (4-amino-2-chlorophenyl)-[4-[(3aS,6aR)-2-methyl-1,3,3a,4,6,6a-hexahydropyrrolo[3,4-c]pyrrole-5-carbonyl]piperazino]methanone

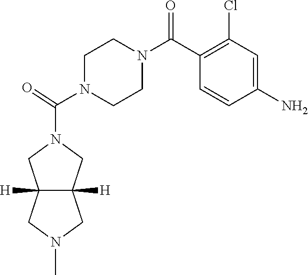 [SMILES: CN1C[C@@H]2CN(C(=O)N3CCN(C(=O)c4ccc(N)cc4Cl)CC3)C[C@@H]2C1]

### Step 1: [4-[(3aS,6aR)-2-methyl-1,3,3a,4,6,6a-hexahydropyrrolo[3,4-c]pyrrole-5-carbonyl]piperazino]-(2-chloro-4-nitrophenyl)methanone

To a solution/suspension of (2-chloro-4-nitrophenyl)-piperazino-methanone (intermediate 21, 0.500 g, 1.82 mmol, 1.00 eq) in dichloromethane (15 mL) was added triphosgene (215.67 mg, 726.79 μmol, 0.400 eq). After 20 min Et₃N (1.1 g, 1.52 mL, 10.9 mmol, 6.00 eq) and (3aR,6aS)-5-methyl-2,3,3a,4,6,6a-hexahydro-1H-pyrrolo[3,4-c]pyrrole (354.59 mg, 363.31 μL, 2.73 mmol, 1.50 eq) were added and the reaction was stirred for 1 hour. The reaction was diluted with water and extracted with dichloromethane. The combined organic layers were washed with brine, dried over magnesium sulfate, filtered and concentrated. The residue was purified by silica gel chromatography using CH₂Cl₂/(CH₂Cl₂:MeOH=9:1+0.25% NH₃) as eluent to afford the title compound (678 mg; 88% yield). MS: 422.159 [M+H]⁺, ESI pos.

### Step 2: [4-[(3aS,6aR)-2-methyl-1,3,3a,4,6,6a-hexahydropyrrolo[3,4-c]pyrrole-5-carbonyl]piperazino]-(4-amino-2-chlorophenyl)methanone

[4-[(3aS,6aR)-2-methyl-1,3,3a,4,6,6a-hexahydropyrrolo[3,4-c]pyrrole-5-carbonyl]piperazino]-(2-chloro-4-nitrophenyl)methanone (678 mg, 1.61 mmol, 1.000 eq) was dissolved in ethanol (10 mL) and water (3.5 mL). Then zinc (1.05 g, 16.1 mmol, 10.0 eq) and ammonium chloride (1.72 g, 32.1 mmol, 20.0 eq) were added, the reaction was heated to 50° C. and stirred 15 hours. The reaction was filtered and washed with ethyl acetate. The mother liquor was concentrated and the residue was diluted with ethyl acetate and water, extracted with sequentially with ethyl acetate and dichloromethane. Both the organic layers and the aq. layer were concentrated and then combined. This residue was treated with dichloromethane and methanol and stirred for 30 min. The mixture was was filtered, the mother liquor dried with MgSO₄, filtered again and concentrated to afford the title compound (492 mg, 62% yield). MS: 92.185 [M+H]⁺, ESI pos. The material was used without further purification.

### Intermediate 24

### (3aR,6aS)-2-[4-(4-amino-2-chlorobenzoyl)piperazine-1-carbonyl]-1,3,3a,4,6,6a-hexahydropyrrolo[3,4-c]pyrrole-5-carboxylic acid tert-butyl ester

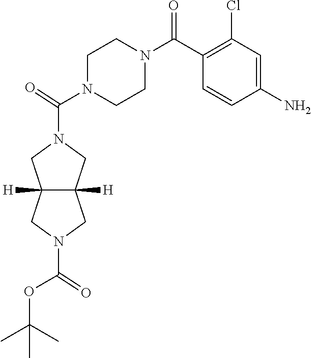 [SMILES: CC(C)(C)OC(=O)N1C[C@@H]2CN(C(=O)N3CCN(C(=O)c4ccc(N)cc4Cl)CC3)C[C@@H]2C1]

### Step 1: (3aR,6aS)-2-[4-(2-chloro-4-nitrobenzoyl)piperazine-1-carbonyl]-1,3,3a,4,6,6a-hexahydropyrrolo[3,4-c]pyrrole-5-carboxylic acid tert-butyl ester

(2-chloro-4-nitrophenyl)-piperazinomethanone (intermediate 21, 0.540 g, 1.96 mmol, 1.000 eq) and DIEA (1.52 g, 2.06 mL, 11.8 mmol, 6.00 eq) were dissolved in dichloromethane (7 mL) and cooled to 0° C. Then triphosgene (232.93 mg, 784.93 μmol, 0.400 eq) was added and the reaction stirred for 30 min. Then (3aR,6aS)-2,3,3a,4,6,6a-hexahydro-1H-pyrrolo[3,4-c]pyrrole-5-carboxylic acid tert-butyl ester (877.02 mg, 3.92 mmol, 2.000 eq) were added and stirring continued at 0° C. for 45 minutes. The reaction was diluted with water and extracted with dichloromethane. The combined organic layers were washed with brine, dried over magnesium sulfate, filtered and concentrated under reduced pressure. The residue was purified by silica gel chromatography using CH₂Cl₂/(CH₂Cl₂:MeOH=9:1+0.25% NH₃) as eluent to afford the title compound (645 mg; 64% yield). MS: 552.2 [M+HCOO]⁻, ESI neg.

### Step 2: (3aR,6aS)-2-[4-(4-amino-2-chlorobenzoyl)piperazine-1-carbonyl]-1,3,3a,4,6,6a-hexahydropyrrolo[3,4-c]pyrrole-5-carboxylic acid tert-butyl ester

(3aR,6aS)-2-[4-(2-chloro-4-nitrobenzoyl)piperazine-1-carbonyl]-1,3,3a,4,6,6a-hexahydropyrrolo[3,4-c]pyrrole-5-carboxylic acid tert-butyl ester (630 mg, 1.23 mmol, 1.00 eq) was dissolved in ethanol (8 mL) and water (2.5 mL). Then zinc (802.75 mg, 12.28 mmol, 10.00 eq) and ammonium chloride (1.31 g, 24.6 mmol, 20.0 eq) were added and the reaction heated to 50° C. for 15 hours. The mixture was solid was filtered and washed with ethyl acetate. The mother liquor was concentrated and the residue was extracted using ethyl acetate and water. The organic layer was washed with brine, dried with MgSO₄, filtered and evaporated to afford the title compound (591 mg, 96% yield). MS: 522.1 [M+HCOO]⁻, ESI neg.

### Intermediate 25

### (4-amino-2-chlorophenyl)-[4-(4-methylpiperazine-1-carbonyl)piperazino]methanone

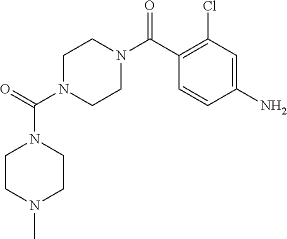 [SMILES: CN1CCN(C(=O)N2CCN(C(=O)c3ccc(N)cc3Cl)CC2)CC1]

### Step 1:(2-chloro-4-nitrophenyl)-[4-(4-methylpiperazine-1-carbonyl)piperazino]methanone

To a mixture of (2-chloro-4-nitrophenyl)-piperazino-methanone (intermediate 21, 500 mg, 1.82 mmol, 1.00 eq) in dichloromethane (5 mL) was added DIEA (1.06 g, 1.43 mL, 8.18 mmol, 4.50 eq) at ambient temperature followed by triphosgene (215.67 mg, 726.79 μmol, 0.400 eq) at 0° C. After 20 min 1-methylpiperazine (363.98 mg, 404.42 μL, 3.63 mmol, 2.000 eq) was added and the mixture was stirred at 0° C. for 30 min. The cooling bath was removed and the reaction was stirred at room temperature overnight. The reaction was diluted with water and extracted with dichloromethane. The combined organic layers were washed with brine, dried over magnesium sulfate, filtered and concentrated. The crude material was purified by silica gel chromatography using dichloromethane/(methanol+0.25% NH₄OH) as eluent to afford the title compound (478 mg; 66% yield). MS: m/z=396.1 [M+H]⁺, ESI pos.

### Step 2: (4-amino-2-chlorophenyl)-[4-(4-methylpiperazine-1-carbonyl)piperazino]methanone

(2-chloro-4-nitrophenyl)-[4-(4-methylpiperazine-1-carbonyl)piperazino]methanone (458 mg, 1.16 mmol, 1.00 eq) was dissolved in ethanol (8 mL) and water (2 mL). Zinc (756.47 mg, 11.57 mmol, 10.000 eq) and ammonium chloride (1.24 g, 23.1 mmol, 20.0 eq) were added. The grayish suspension was heated to 50° C. and stirred 15 hours. The solid was filtered and washed with ethyl acetate. The mother liquor was concentrated and the residue was extracted using ethyl acetate and water. The organic phase was washed with brine, dried with MgSO₄, filtered and concentrated to afford a first batch of the title compound (206 mg, 49% yield). The aqueous layer was extracted several times with dichloromethane. The combined dichloromethane layers were dried with MgSO₄, filtered and concentrated to afford another batch of the title compound (194 mg, 46% yield). MS: m/z=366.17 [M+H]⁺, ESI pos.

The following compounds were prepared in analogy to the procedure above

| Int | Structure | Name | Reagents | MS |
|---|---|---|---|---|
| 26 | 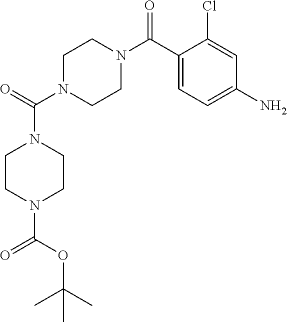 [SMILES: CC(C)(C)OC(=O)N1CCN(C(=O)N2CCN(C(=O)c3ccc(N)cc3Cl)CC2)CC1] | 4-[4-(4-amino-2-chloro- benzoyl)piperazine-1- carbonyl]piperazine-1- carboxylic acid tert- butyl ester | Step 1) 1-Boc- piperazine, reaction at 0° C. only, step 2) silica gel chromatography after extraction | m/z = 452.2 [M + H]+, ESI pos |

### Intermediate 27

### (4-amino-2-chlorophenyl)-[4-(morpholine-4-carbonyl)piperazino]methanone

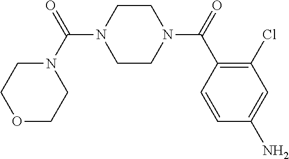 [SMILES: Nc1ccc(C(=O)N2CCN(C(=O)N3CCOCC3)CC2)c(Cl)c1]

### Step 1: (2-chloro-4-nitrophenyl)-[4-(morpholine-4-carbonyl)piperazino]methanone

To a clear solution of (2-chloro-4-nitrophenyl)-piperazino-methanone (intermediate 21, 50 mg, 184 μmol, 1.00 eq) in dichloromethane (2 mL) was added triphosgene (21.79 mg, 73.42 μmol, 0.400 eq) at ambient temperature. After 20 min morpholine (31.98 mg, 31.98 μL, 367.1 μmol, 2.000 eq) was added to the white suspension and the mixture was stirred for 20 min at ambient temperature. The reaction was diluted with water and extracted with ethyl acetate. The combined organic layers were combined, washed with brine, dried over magnesium sulfate, filtered and concentrated under reduced pressure. The residue was purified twice by silica gel chromatography using CH₂Cl₂/(MeOH+0.25% NH₃) as eluent to afford the title compound (19 mg, 27% yield). MS: m/z=383.2 [M+H]⁺.

### Step 2: (4-amino-2-chlorophenyl)-[4-(morpholine-4-carbonyl)piperazino]methanone

(2-chloro-4-nitrophenyl)-[4-(morpholine-4-carbonyl)piperazino]methanone (19 mg, 48 μmol, 1.0 eq) was dissolved in ethanol (1 mL) and water (300 μL) and ammonium chloride (65.05 mg, 1.22 mmol, 25.0 eq) and zinc (31.8 mg, 486 μmol, 10.0 eq) were added. The mixture was heated to 50° C. for 4 h. Again zinc (31.8 mg, 486 μmol, 10.0 eq) and ammonium chloride (65.05 mg, 1.22 mmol, 25.00 eq) were added and stirring continued at 50° C. for 1 hour. The reaction mixture was filtered and washed using ethyl acetate and the obtained solution concentrated. The residue was extracted using ethyl acetate and water, the organic phase was washed with brine, dried with MgSO₄, filtered and concentrated to afford the title compound (19 mg, quant.), which was used without further purification. MS: m/z=353.2 [M+H]⁺.

### Intermediate 28

### 4-(4-amino-2-chlorobenzoyl)piperazine-1-carboxylic acid 9H-fluoren-9-ylmethyl ester

To a solution of 4-amino-2-chlorobenzoic acid (2.5 g, 14 mmol, 1.0 eq) in DMF, extra dry (26 mL) was added fmoc-piperazine hydrochloride (5.02 g, 14.6 mmol, 1.00 eq), DIPEA (5.65 g, 7.63 mL, 43.7 mmol, 3.00 eq) and HATU (8.31 g, 21.9 mmol, 1.50 eq). The clear solution stirred at RT for 15 hours. The reaction mixture was concentrated under reduced pressure, the residue diluted with water and extracted 3 times with ethyl acetate. The combined organic layers were washed 2 times with water and brine, dried over magnesium sulfate, filtered and concentrated under reduced pressure. The residue was triturated with dichloromethane, the suspension filtered and the mother liquor concentrated. The residue was purified by silica gel chromatography using dichloromethane/(dichloromethane/methanol+0.2 5% NH₄OH 9:1) as eluent. The obtained material was again purified by silica gel chromatography using ethyl acetate/heptane as eluent to afford the title compound (3.81 g, 54% yield). MS: 462.2 [M+H]⁺, ESI pos.

### Intermediate 29

### (1R,5S,6r)-3-ethyl-3-azabicyclo[3.1.0]hexane-6-carboxylic acid; hydrochloride

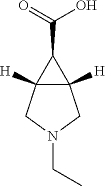

### Step 1: (1R,5S,6r)-3-ethyl-3-azabicyclo[3.1.0]hexane-6-carboxylic acid methyl ester

In a sealed tube (1R,5S,6r)-3-azabicyclo[3.1.0]hexane-6-carboxylic acid methyl ester (100 mg, 354 μmol, 1.00 eq) [1024038-72-9] was dissolved in ethanol (1 mL) and then K₂CO₃ (146.85 mg, 1.06 mmol, 3.000 eq) and ethyl iodide (110.48 mg, 57.25 μL, 708.37 μmol, 2.000 eq) were added at ambient temperature. The mixture was heated up to 85° C. for 1 hour. The suspension was filtered and washed with ethanol. The mother liquor was concentrated under reduced pressure, the residue was adsorbed on isolute HM-N, and purified by silica gel chromatography using CH₂Cl₂/(CH₂Cl₂:MeOH=9:1+0.25% NH₃) as eluent to afford the title compound (24 mg, 38% yield). MS: m/z=170.1 [M+H]⁺, ESI pos.

### Step 2: (1R,5S,6r)-3-ethyl-3-azabicyclo[3.1.0]hexane-6-carboxylic acid. 1:1 hydrogen chloride

To a mixture of (1R,5S,6r)-3-ethyl-3-azabicyclo[3.1.0]hexane-6-carboxylic acid methyl ester (24 mg, 135 μmol, 1.000 eq) in tetrahydrofuran (1 mL) was added 1 M LiOH solution in water (431.59 μL, 431.59 μmol, 3.203 eq) and LiOH·H₂O (67.95 mg, 1.62 mmol, 12.00 eq). The reaction was stirred at ambient temperature for 1.5 h. The mixture was acidified using 25% aq. HCl (314.4 mg, 262. μL, 2.16 mmol, 16.0 eq) and then concentrated under reduced pressure to afford the title compound, which was used without further purification (30 mg, quant.). MS: m/z=156.1 [M+H]⁺, ESI pos.

### Intermediate 30

### (1R,3s,5S,6r)-3-(benzyloxycarbonylamino)bicyclo[3.1.0]hexane-6-carboxylic acid

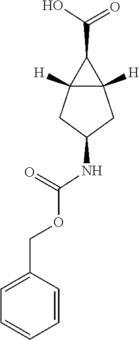 [SMILES: O=C(N[C@@H]1C[C@@H]2[C@H](C1)[C@H]2C(=O)O)OCc1ccccc1]

### Step 1:2-cyclopent-3-en-1-ylisoindoline-1,3-dione

To a stirred mixture of cyclopent-3-en-1-ol (24 g, 285 mmol, 1 equiv), phthalimide (83.95 g, 570.6 mmol, 2.0 eq) and triphenylphosphine (149.67 g, 570.62 mmol, 2 equiv) in THF (480 mL) was added diisopropyl azodicarboxylate (132.69 g, 656.22 mmol, 2.3 equiv) dropwise at 0° C. under nitrogen atmosphere. The resulting mixture was stirred for 16 h at room temperature under nitrogen atmosphere. The reaction was quenched by the addition of water/ice (1 L) at 0° C. The resulting mixture was extracted with EtOAc (3×500 mL). The combined organic layers were washed with brine (2×1 L), dried over anhydrous Na₂SO₄. After filtration, the filtrate was concentrated under reduced pressure. The residue was purified by reverse-phase flash chromatography with the following conditions: column, C18 silica gel; mobile phase, MeCN in Water (0.1% TFA) to afford the title compound (46 g, 76% yield). MS: m/z=214.1 [M+H]⁺, ESI pos.

### Step 2: ethyl (1R,3s,5S,6r)-3-(1,3-dioxoisoindolin-2-yl)bicyclo[3.1.0]hexane-6-carboxylate

A mixture of 2-(cyclopent-3-en-1-yl) isoindole-1,3-dione (46 g, 220 mmol, 1 equiv) and Rh2 (OAc) 4 (2.86 g, 6.47 mmol, 0.03 equiv) in DCM (460 mL) was stirred for 20 min at room temperature under nitrogen atmosphere. To the above mixture was added ethyl diazoacetate (98.46 g, 862.9 mmol, 4 equiv) dropwise over 12 h at room temperature. The resulting mixture was stirred for an additional 2 h at room temperature. The resulting mixture was filtered, the filter cake was washed with DCM (2×50 mL). The filtrate was concentrated under reduced pressure. The residue was purified by silica gel column chromatography, eluted with PE/EA (10:1) to afford the title compound (35 g, 54% yield). MS: m/z=300.2 [M+H]⁺, ESI pos.

### Step 3: ethyl (1R,3s,5S,6r)-3-aminobicyclo[3.1.0]hexane-6-carboxylate

A mixture of ethyl (1R,3s,5S,6r)-3-(1,3-dioxoisoindol-2-yl)bicyclo[3.1.0]hexane-6-carboxylate (65 g, 217 mmol, 1 equiv) and NH₂NH₂·H₂O (62.12 g, 868.62 mmol, 4 equiv, 70%) in EtOH (650 mL) was stirred for 16 h at 68° C. The mixture was allowed to cool down to room temperature. The resulting mixture was concentrated under reduced pressure to afford the title compound (34 g, 92% yield). MS: m/z=170.2 [M+H]⁺, ESI pos.

### Step 4: ethyl (1S,3s,5R,6r)-3-(benzyloxycarbonylamino)bicyclo[3.1.0]hexane-6-carboxylate

To a stirred mixture of ethyl (1R,3s,5S,6r)-3-aminobicyclo[3.1.0]hexane-6-carboxylate (33 g, 195 mmol, 1 equiv) and DIEA (50.41 g, 390.0 mmol, 2 equiv) in DCM (660 mL) was added benzyl chloroformate (36.59 g, 214.5 mmol, 1.1 equiv) dropwise at 0° C. under nitrogen atmosphere. The resulting mixture was stirred for 16 h at room temperature under nitrogen atmosphere. The resulting mixture was diluted with water (300 mL). The resulting mixture was extracted with EtOAc (3×100 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EA (5:1) to afford the title compound (43 g, 73% yield). MS: m/z=276.1 [M+H]⁺, ESI pos.

### Step 5: (1R,3s,5S,6r)-3-(benzyloxycarbonylamino)bicyclo[3.1.0]hexane-6-carboxylic acid

To a stirred mixture of ethyl (1R,3s,5S,6r)-3-{[(benzyloxy)carbonyl]amino}bicyclo[3.1.0]hexane-6-carboxylate (16 g, 52.7 mmol, 1 equiv) in THF (160 mL) was added LiOH (3.16 g, 132 mmol, 2.5 equiv) in water (40 mL) dropwise at room temperature. The resulting mixture was stirred for 16 h at 70° C. The mixture was allowed to cool down to room temperature. The resulting mixture was concentrated under reduced pressure. The residue was dissolved in water (100 mL). The mixture was acidified to pH 5 with 1 M HCl. The precipitated solids were collected by filtration and washed with water (3×10 mL). The crude product was re-crystallized from MeOH (250 mL) to afford (1R,3s,5S,6r)-3-{[(benzyloxy)carbonyl]amino}bicyclo[3.1.0]hexane-6-carboxylic acid (7.5 g, 52% yield). MS: m/z=276.2 [M+H]⁺, ESI pos.

### Intermediate 31

### rac-tert-butyl (3R,4R)-3-hydroxy-4-(piperazine-1-carboxamido)pyrrolidine-1-carboxylate

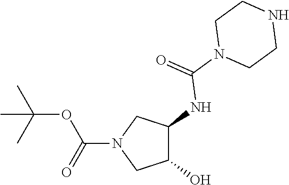 [SMILES: CC(C)(C)OC(=O)N1C[C@@H](O)[C@H](NC(=O)N2CCNCC2)C1]

### Step 1: rac-benzyl 4-(((3R,4R)-1-(tert-butoxycarbonyl)-4-hydroxypyrrolidin-3-yl)carbamoyl)piperazine-1-carboxylate

A solution of rac-tert-butyl (3R,4R)-3-amino-4-hydroxypyrrolidine-1-carboxylate (5.05 g, 25 mmol, 2.5 eq) and triethylamine (3.03 g, 4.18 ml, 30 mmol, 3 eq) in DMF (49.9 ml) was added CDI (4.05 g, 25 mmol, 2.5 eq) while cooling with an ice bath (temperature rose from 4° C. to 17° C.). Then the cooling bath was removed and the reaction mixture was stirred at RT for 20 minutes. Then benzyl piperazine-1-carboxylate (2.2 g, 9.99 mmol, 1 eq) was added and stirring at RT continued for 4 hours. The reaction mixture was concentrated under reduced pressure. The residue was dissolved in ethyl acetate and diluted with water. The mixture was extracted twice with ethyl acetate and the organic layers were washed once with brine. The combined organic layers were dried with sodium sulfate, filtered and concentrated under reduced pressure. The crude material was purified by silica gel chromatography using dichloromethane/methanol as eluent to afford the title compound (4.34 g, 6.58 mmol, 65.9% yield) which was used without further purification. MS: m/z=449.239 [M+H]⁺, ESI pos.

### Step 2: rac-tert-butyl (3R,4R)-3-hydroxy-4-(piperazine-1-carboxamido)pyrrolidine-1-carboxylate

A flask containing a solution of rac-benzyl 4-(((3R,4R)-1-(tert-butoxycarbonyl)-4-hydroxypyrrolidin-3-yl)carbamoyl)piperazine-1-carboxylate (4.34 g, 6.58 mmol, 1 eq) in methanol (21.9 ml) was evacuated three times and flushed with argon. Then palladium on carbon 10% (700 mg, 658 μmol, 0.1 eq) was added. The flask was evacuated three times and flushed with argon. Then the flask was evacuated three times and flushed with H₂. The reaction was stirred at RT under H₂ for 3 hours. The reaction mixture was filtered over a satorius filter and washed with methanol. The filtrate was concentrated under reduced pressure to afford the title compound (3.12 g, 6.55 mmol, 99.5% yield) which was used without further purification. MS: m/z=315.203 [M+H]⁺, ESI pos.

### Intermediate 32

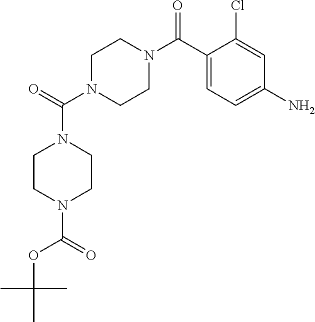 [SMILES: CC(C)(C)OC(=O)N1CCN(C(=O)N2CCN(C(=O)c3ccc(N)cc3Cl)CC2)CC1]

4-[4-(2-chloro-4-nitro-benzoyl)piperazine-1-carbonyl]piperazine-1-carboxylic acid tert-butyl ester (1.01 g, 2.1 mmol, 1.00 eq) was dissolved/suspended in ethanol (12 mL) and water (3 mL). Zinc (1.37 g, 21.0 mmol, 10.0 eq) and ammonium chloride (1.68 g, 31.4 mmol, 15.0 eq) were added. The grayish suspension was heated to 50° C. and stirred overnight. LC-MS after 15 h showed the reaction was complete. The solid was filtered and washed with ethyl acetate. The mother liquor was evaporated and the residue was extracted using ethyl acetate and water. The organic phase was washed with brine, dried with MgSO₄, filtered and evaporated to afford 1000 mg. It was purified by chromatography (SiO₂; Silicycle SiliaSep PREMIUM 24 g; gradient heptane/ethyl acetate to ethyl acetate) to yield the title compound (770 mg, 80% yield). MS [M+H]⁺: 452.2

### Intermediate 33

### 2-(difluoromethyl)-3-fluoro-4-(fluoromethoxy)aniline

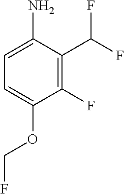 [SMILES: Nc1ccc(OCF)c(F)c1C(F)F]

### Step 1 6-bromo-2-fluoro-3-(fluoromethoxy)benzaldehyde

To a solution of 6-bromo-2-fluoro-3-hydroxy-benzaldehyde (45 g, 205 mmol, 1.0 eq) and K₂CO₃ (31.24 g, 226.0 mmol, 1.1 eq) in MeCN (600 mL) was added bromo (fluoro) methane (46.41 g, 410.9 mmol, 2.0 eq) at 0° C. The mixture was then warmed to 20° C. and stirred for 60 h. The mixture was filtered and concentrated under reduced pressure. The residue was purified by silica column (EtOAc in PE: 1-30%) to afford the title compound (43 g, 171 mmol, 83% yield). 1H NMR (400 MHz, CDCl₃) δ 10.32 (s, 1H), 7.45 (dd, J=1.6, 8.8 Hz, 1H), 7.34-7.28 (m, 1H), 5.80-5.64 (m, 2H) ppm.

### Step 2 1-bromo-2-(difluoromethyl)-3-fluoro-4-(fluoromethoxy)benzene

To a solution of 6-bromo-2-fluoro-3-(fluoromethoxy)benzaldehyde (43 g, 171 mmol, 1.0 eq) in CH₂Cl₂ (500 mL) was added dropwise diethylaminosulphur trifluoride (67.89 mL, 513.9 mmol, 3.0 eq) at 0° C. The resulting solution was then stirred at 20° C. for 2 h. The mixture was quenched with saturated NaHCO₃ (1500 mL), extracted with EtOAc (2×1000 mL), and washed with brine (500 mL). The organic layer was dried over Na₂SO₄ and concentrated under reduced pressure. The residue was purified by column (PE:EtOAc 1:0 to 4:1) to afford the title compound (43 g, 158 mmol, 92% yield) as a light brown oil. 1H NMR (400 MHz, CDCl₃) δ=7.38 (td, J=0.8, 9.0 Hz, 1H), 7.26-7.18 (m, 1H), 7.02 (dt, J=0.8, 52.8 Hz, 1H), 5.71 (d, J=53.6 Hz, 2H)

### Step 3 N-[2-(difluoromethyl)-3-fluoro-4-(fluoromethoxy)phenyl]-1,1-diphenyl-methanimine

A solution of 1-bromo-2-(difluoromethyl)-3-fluoro-4-(fluoromethoxy)benzene (43 g, 158 mmol, 1.0 eq), diphenylmethanimine (26.43 mL, 157.5 mmol, 1.0 eq), 9,9-dimethyl-4,5-bis(diphenylphosphino)xanthene (9.11 g, 15.8 mmol, 0.1 eq), tris(dibenzylideneacetone)dipalladium(0)(14.42 g, 15.75 mmol, 0.1 eq) and Cs₂CO₃ (102.63 g, 315.0 mmol, 2.0 eq) in 1,4-dioxane (500 mL) was stirred at 90° C. for 16 h under N₂. The mixture was filtered, and the filtrate was concentrated under reduced pressure. The residue was purified by column (PE:EtOAc 1:0 to 2:1) to afford the title compound (55 g, 147.31 mmol, 94% yield). MS: m/z=374.1. [M+H]+, ESI pos.

### Step 4 2-(difluoromethyl)-3-fluoro-4-(fluoromethoxy)aniline

A solution of N-[2-(difluoromethyl)-3-fluoro-4-(fluoromethoxy)phenyl]-1,1-diphenyl-methanimine (35 g, 93.8 mmol, 1.0 eq) in CH₂Cl₂ (200 mL) was added HCl (23.44 mL, 12 M) and stirred at 25° C. for 16 h. The reaction mixture was filtered, and the filtrate was concentrated under reduced pressure. The residue was purified by reverse phase HPLC (H₂O (0.1% HCl)-MeCN 40-55% B) to afford the title compound (23 g, 110 mmol, 75% yield). MS: m/z=210.0. [M+H]+, ESI pos.

The following compound was prepared in analogy to the procedure above

| Int | Structure | Name | Reagents | MS [M + H]+ |
|---|---|---|---|---|
| 34 | 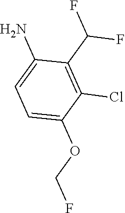 [SMILES: Nc1ccc(OCF)c(Cl)c1C(F)F] | 3-chloro-2- (difluoromethyl)-4- (fluoromethoxy) aniline | 6-bromo-2-chloro- 3-hydroxy- benzaldehyde | 226.1 |

### Intermediate 35

### 4-(cyclopropoxy)-2-(difluoromethyl)-3-fluoro-aniline

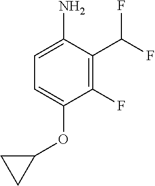 [SMILES: Nc1ccc(OC2CC2)c(F)c1C(F)F]

### Step 1: 4-bromo-1-(cyclopropoxy)-2-fluoro-benzene

To a solution of 4-bromo-2-fluorophenol (10 g, 52.36 mmol, 1.0 eq) in NMP (100 mL) was added cyclopropyl bromide (7.6 g, 62.83 mmol, 1.2 eq), cesium carbonate (18.76 g, 57.59 mmol, 1.1 eq) and NaI (7.85 g, 52.36 mmol, 1.0 eq) at 0° C., then the mixture was warmed to 20° C. and stirred at 180° C. for 5 h The reaction mixture was poured into 1N HCl water (100 mL), extracted with EtOAc (200 mL×3). Combined extracts were washed with brine (200 mL), dried over Na₂SO₄, filtered, and concentrated under vacuum. The residue was purified by silica gel chromatography to afford the title compound (3.8 g, 16.45 mmol, 25.13% yield). 1H NMR (400 MHz, CHLOROFORM-d) δ=7.25-7.13 (m, 3H), 3.82-3.76 (m, 1H), 0.86-0.78 (m, 4H).

### Step 2: 6-bromo-3-(cyclopropoxy)-2-fluoro-benzaldehyde

A mixture of 4-bromo-1-(cyclopropoxy)-2-fluoro-benzene (30 g, 129.84 mmol, 1.0 eq) in THF (500 mL) was evacuated and backfilled with N2. Then the solution was cooled to −78° C. and added dropwise dimethylformamide (40.22 mL, 519.35 mmol, 4.0 eq). Then lithium diisopropylamide (149.31 mL, 298.62 mmol, 2.3 eq) was added dropwise and the reaction stirred at −78° C. for 0.5 h. then warmed up to −40° C. for 2 h. The reaction mixture was quenched by dropwise addition of sat.aq NH₄Cl (400 mL) under N₂, then extracted with EtOAc (1000 mL). Combined extracts were washed with brine (1000 mL), dried over Na₂SO₄, filtered, and concentrated under vacuum. The residue was purified by silica gel chromatography (petroleum ether/ethyl acetate=1:0˜4:1) to afford the title compound (2.6 g, 10.04 mmol, 10.54% yield). 1H NMR (400 MHz, CHLOROFORM-d) δ=10.32 (s, 1H), 7.44-7.32 (m, 2H), 3.90-3.79 (m, 1H), 0.94-0.78 (m, 4H).

### Step 3: 1-bromo-4-(cyclopropoxy)-2-(difluoromethyl)-3-fluoro-benzene

To a solution of 6-bromo-3-(cyclopropoxy)-2-fluoro-benzaldehyde (2.6 g, 10.04 mmol, 1.0 eq) in DCM (30 mL) was added dropwise diethylaminosulfur trifluoride (3.98 mL, 30.11 mmol, 3.0 eq) at 0° C., then the solution was stirred at 20° C. for 2 h. The mixture was quenched with sat. NaHCO₃ (10 mL) and extracted with EtOAc (20 mL×2), washed with brine (20 mL). The combined organic layers were dried over sodium sulfate and concentrated in vacuo. The residue was purified by silica gel chromatography (PE:EA=1:0 to 4:1) to afford the title compound (2.1 g, 7.47 mmol, 74.45% yield) as light brown oil. 1H NMR (400 MHz, CHLOROFORM-d) δ=7.37-7.27 (m, 2H), 7.01 (t, J=52.9 Hz, 1H), 3.90-3.77 (m, 1H), 0.96-0.76 (m, 4H).

### Step 4: N-[4-(cyclopropoxy)-2-(difluoromethyl)-3-fluoro-phenyl]-1,1-diphenyl-methanimine

A mixture of 1-bromo-4-(cyclopropoxy)-2-(difluoromethyl)-3-fluoro-benzene (2.1 g, 7.47 mmol, 1.0 eq) diphenylmethanimine (1.25 mL, 7.47 mmol, 1.0 eq) 9,9-dimethyl-4,5-bis(diphenylphosphino)xanthene (0.43 g, 0.75 mmol, 0.1 eq) and tris(dibenzylideneacetone)dipalladium(0)(0.68 g, 0.75 mmol, 0.1 eq) cesium carbonate (4.87 g, 14.94 mmol, 2.0 eq) in 1,4-dioxane (30 mL) was stirred at 90° C. for 2 h under N2. The mixture was filtered and the filtate was concentrated under. The residue was purified by column chromatography (PE:EA=1:0 to 2:1 afford the title compound (1.7 g, 4.46 mmol, 59.66% yield) as yellow oil.

MS: 382.1 [M+H]+, ESI+

### Step 5: 4-(cyclopropoxy)-2-(difluoromethyl)-3-fluoro-aniline

A solution of N-[4-(cyclopropoxy)-2-(difluoromethyl)-3-fluoro-phenyl]-1,1-diphenyl-methanimine (1.7 g, 4.46 mmol, 1.0 eq) in DCM (5 mL) was added 12M HCl (5.0 mL, 60.0 mmol, 13.46 eq) and stirred at 25° C. for 2 h. The mixture was concentrated under vacuo. The residue was purified by reversed phase chromatography (water (0.1% FA)-ACN B %:60%-80%) to afford 4-(cyclopropoxy)-2-(difluoromethyl)-3-fluoro-aniline (357 mg, 1.64 mmol, 36.88% yield) as brown oil. MS: 218.0 [M+H]+, ESI+

### Intermediate 36

### 3-chloro-2-fluoro-4-(fluoromethoxy)aniline

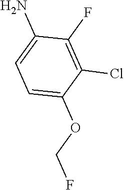 [SMILES: Nc1ccc(OCF)c(Cl)c1F]

### Step 1: 2-chloro-3-fluoro-1-(fluoromethoxy)-4-nitrobenzene

To a mixture of 2-chloro-3-fluoro-4-nitrophenol ([1632444-57-5], 1.1 g, 5.7 mmol, 1.0 eq) and K₂CO₃ (2381 mg, 17.23 mmol, 3.0 eq) in MeCN (12 mL) was added bromofluoromethane (3243 mg, 28.71 mmol, 5.0 eq) at 0° C. The mixture was then stirred at 10° C. for 16 h. The mixture was filtered, and the filtrate was concentrated under reduced pressure. The residue was purified on SiO₂ (EtOAc:PE 0:1 to 5:1) to afford the title compound (1 g, 4.5 mmol, 78% yield) as yellow oil. 1H NMR (400 MHz, CDCl₃) δ 8.08 (dd, J=8.0, 9.2 Hz, 1H), 7.11 (td, J=1.2, 9.2 Hz, 1H), 5.87 (d, J=52.4 Hz, 2H) ppm.

### Step 2: 3-chloro-2-fluoro-4-(fluoromethoxy)aniline

To a solution of 2-chloro-3-fluoro-1-(fluoromethoxy)-4-nitrobenzene (900 mg, 4.03 mmol, 1.0 eq) in EtOH (10 mL) were added Fe (1.12 g, 20.13 mmol, 5.0 eq) and NH₄Cl (1.08 g, 20.13 mmol, 5.0 eq) at 20° C. The reaction mixture was then stirred at 60° C. for 1 h. The mixture was filtered and washed with EtOAc (30 mL), the filtrate was poured into H₂O (100 ml) and extracted with EtOAc (3×30 mL). The combined organic phase was washed with brine (3×30 mL), dried over Na₂SO₄, filtered and concentrated under reduced pressure. The residue was purified by column chromatography (SiO₂, PE:EtOAc 1:0 to 3:1) to afford the title compound (650 mg, 3.36 mmol, 83% yield).

MS: m/z=193.9 [M+H]+, ESI pos.

### Intermediate 37

### 3-(trifluoromethyl)-1-trityl-pyrazol-4-amine

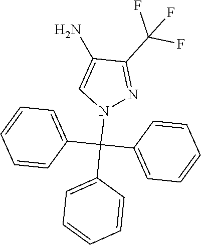 [SMILES: Nc1cn(C(c2ccccc2)(c2ccccc2)c2ccccc2)nc1C(F)(F)F]

### Step 1 4-nitro-3-(trifluoromethyl)-1-trityl-pyrazole

To a solution of 4-nitro-3-(trifluoromethyl)-1H-pyrazole (18 g, 99.4 mmol, 1.0 eq) and triethylamine (13.86 mL, 99.41 mmol, 1.0 eq) in DMF (250 mL) was added triphenylmethyl chloride (27.71 g, 99.41 mmol, 1.0 eq) at 0° C. The mixture was then stirred at 20° C. for 16 h. The mixture was diluted H₂O (500 mL) and then extracted with EtOAc (3×400 mL). The combined organic layers were washed with brine (3×150 mL), dried over sodium Na₂SO₄, the filtrate was concentrated and purified by silica gel column chromatography (PE:EtOAc 1:0 to 5:1) to give the title compound (48 g, 113.37 mmol, 91% yield). 1H NMR (400 MHz, CDCl₃) δ 8.18 (s, 1H), 7.43-7.36 (m, 9H), 7.14-7.09 (m, 6H) ppm.

### Step 2 3-(trifluoromethyl)-1-trityl-pyrazol-4-amine

To a stirred solution of NiCl2 (5.82 g, 44.87 mmol, 0.5 eq) in MeOH (400 mL) was added sodium borohydride (1.7 g, 44.87 mmol, 0.5 eq) in portions at 0° C. After stirred at 0° C. for 0.5 h, 4-nitro-3-(trifluoromethyl)-1-trityl-pyrazole (38.0 g, 89.75 mmol, 1.0 eq) was added to the reaction mixture at 0° C. Following by sodium borohydride (10.19 g, 269.25 mmol, 3.0 eq) was added in portions at 0° C. and stirred at 25° C. for 16 h. The mixture was filtered, the solid was washed with EtOAc (4×300 mL). The combined filtrate was diluted with H₂O (1000 mL) and then extracted with EtOAc (3×10⁰ mL). The combined organic layers were washed with brine (100 mL), dried over Na₂SO₄, concentrated and purified by column (PE:EtOAc 1:0˜1:1) to give the title compound (24 g, 61.0 mmol, 68% yield). MS: m/z=394.2. [M+H]⁺, ESI pos.

### Intermediate 38

### ethyl 5-[bis(tert-butoxycarbonyl)amino]-1-[2-(difluoromethyl)-3-fluoro-4-(fluoromethoxy)phenyl]imidazole-4-carboxylate

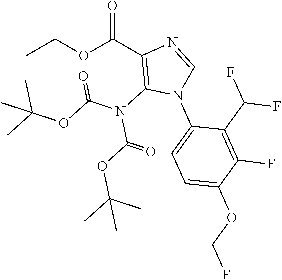 [SMILES: CCOC(=O)c1ncn(-c2ccc(OCF)c(F)c2C(F)F)c1N(C(=O)OC(C)(C)C)C(=O)OC(C)(C)C]

### Step 1 ethyl 5-amino-1-[2-(difluoromethyl)-3-fluoro-4-(fluoromethoxy)phenyl]imidazole-4-carboxylate

A solution of ethyl 2-amino-2-cyanoacetate; 4-methylbenzene-1-sulphonic acid (41.29 g, 137.47 mmol, 1.25 eq) and triethylamine (19.16 mL, 137.47 mmol, 1.25 eq) in trimethyl orthoformate (250 mL, 109.97 mmol, 1.0 eq) was stirred at 90° C. for 16 h. The solution was cooled to 25° C. concentrated under reduced pressure. The residue was dissolved in MeCN (200 mL), 2-(difluoromethyl)-3-fluoro-4-(fluoromethoxy)aniline (Intermediate 33, 23 g, 110 mmol, 1.0 eq) was added. The resulting mixture was stirred at 90° C. for another 16 h. The mixture was concentrated and purified by column (SiO₂, PE:EtOAc 0:1) to give the title compound (5.35 g, 15.41 mmol, 14% yield). MS: m/z=348.1. [M+H]+, ESI pos.

### Step 2 ethyl 5-[bis(tert-butoxycarbonyl)amino]-1-[2-(difluoromethyl)-3-fluoro-4-(fluoromethoxy)phenyl]imidazole-4-carboxylate

To a solution of ethyl 5-amino-1-[2-(difluoromethyl)-3-fluoro-4-(fluoromethoxy)phenyl]imidazole-4-carboxylate (5 g, 14.4 mmol, 1.0 eq), DMAP (0.88 g, 7.2 mmol, 0.5 eq), triethylamine (6.02 mL, 43.19 mmol, 3.0 eq) in CH₂Cl₂ (60 mL) was added Boc₂O (12.57 g, 57.59 mmol, 4.0 eq) at 0° C. under N₂. The mixture was then stirred at 25° C. for 3 h. The mixture was concentrated and purified by column (PE/EtOAc 1:1˜1:2) to afford the title compound (7 g, 12.79 mmol, 89% yield).

MS: m/z=548.3. [M+H]+, ESI pos.

The following compounds were prepared in analogy to the procedure above

| | | | | MS |
|---|---|---|---|---|
| Int | Structure | Name | Reagents | [M + H]+ |
| 39 | 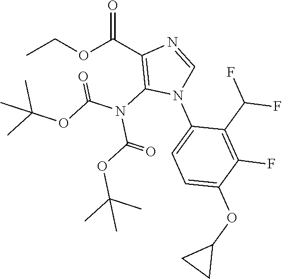 [SMILES: CCOC(=O)c1ncn(-c2ccc(OC3CC3)c(F)c2C(F)F)c1N(C(=O)OC(C)(C)C)C(=O)OC(C)(C)C] | ethyl 5-[bis(tert- butoxycarbonyl)amino]- 1-[4-(cyclopropoxy)-2- (difluoromethyl)-3- fluorophenyl]imidazole- 4-carboxylate | Intermediate 35 | 556.3 |
| 40 | 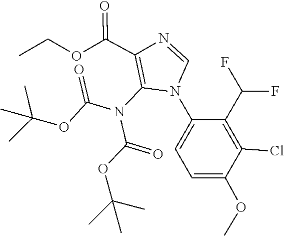 [SMILES: CCOC(=O)c1ncn(-c2ccc(OC)c(Cl)c2C(F)F)c1N(C(=O)OC(C)(C)C)C(=O)OC(C)(C)C] | ethyl 5-[bis(tert- butoxycarbonyl)amino]- 1-[3-chloro-2- (difluoromethyl)-4- methoxyphenyl] imidazole-4-carboxylate | 3-chloro-2- (difluoromethyl)-4- methoxyaniline [CAS: 2385293- 49-0] | 546.2 |
| 41 | 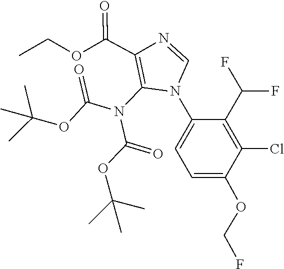 [SMILES: CCOC(=O)c1ncn(-c2ccc(OCF)c(Cl)c2C(F)F)c1N(C(=O)OC(C)(C)C)C(=O)OC(C)(C)C] | ethyl 5-[bis(tert- butoxycarbonyl)amino]- 1-[3-chloro-2- (difluoromethyl)-4- (fluoromethoxy)phenyl] imidazole-4-carboxylate | Intermediate 34 | 564.1 |
| 42 | 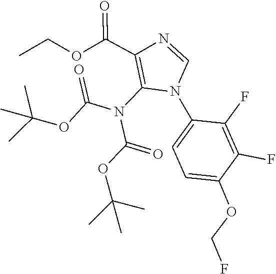 [SMILES: CCOC(=O)c1ncn(-c2ccc(OCF)c(F)c2F)c1N(C(=O)OC(C)(C)C)C(=O)OC(C)(C)C] | ethyl 5-[bis(tert- butoxycarbonyl)amino]- 1-[2,3-difluoro-4- (fluoromethoxy)phenyl] imidazole-4-carboxylate | 2,3-difluoro-4- (fluoromethoxy) aniline | 516.0 |
| 43 | 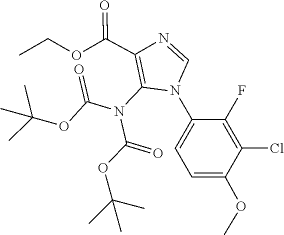 [SMILES: CCOC(=O)c1ncn(-c2ccc(OC)c(Cl)c2F)c1N(C(=O)OC(C)(C)C)C(=O)OC(C)(C)C] | ethyl 5-[bis(tert- butoxycarbonyl)amino]- 1-(3-chloro-2-fluoro-4- methoxyphenyl) imidazole-4-carboxylate | 3-chloro-2-fluoro- 4-methoxyaniline [1936314-06-5] | 514.2 |
| 44 | 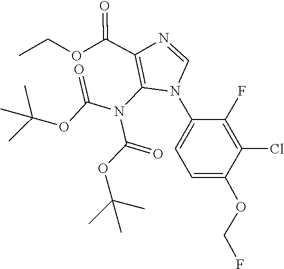 [SMILES: CCOC(=O)c1ncn(-c2ccc(OCF)c(Cl)c2F)c1N(C(=O)OC(C)(C)C)C(=O)OC(C)(C)C] | ethyl 5-[bis(tert- butoxycarbonyl)amino]- 1-[3-chloro-2-fluoro-4- (fluoromethoxy)phenyl] imidazole-4-carboxylate | Intermediate 36 | 532.2 |
| 45 | 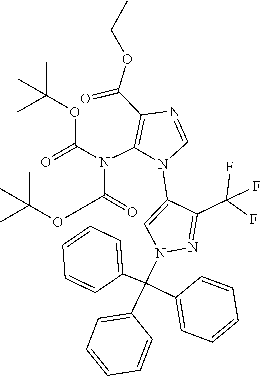 [SMILES: CCOC(=O)c1ncn(-c2cn(C(c3ccccc3)(c3ccccc3)c3ccccc3)nc2C(F)(F)F)c1N(C(=O)OC(C)(C)C)C(=O)OC(C)(C)C] | ethyl 5-[bis(tert- butoxycarbonyl)amino]- 1-[3-(trifluoromethyl)-1- trityl-pyrazol-4- yl]imidazole-4- carboxylate | Intermediate 37 | 732.3 |

### Intermediate 46

### 5-(tert-butoxycarbonylamino)-1-[2-(difluoromethyl)-3-fluoro-4-(fluoromethoxy)phenyl]imidazole-4-carboxylic acid

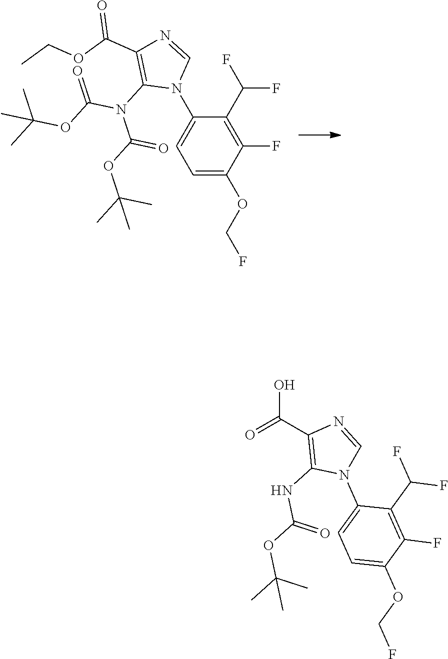 [SMILES: CCOC(=O)c1ncn(-c2ccc(OCF)c(F)c2C(F)F)c1N(C(=O)OC(C)(C)C)C(=O)OC(C)(C)C ; CC(C)(C)OC(=O)Nc1c(C(=O)O)ncn1-c1ccc(OCF)c(F)c1C(F)F]

To a solution of ethyl 5-[bis(tert-butoxycarbonyl)amino]-1-[2-(difluoromethyl)-3-fluoro-4-(fluoromethoxy)phenyl]imidazole-4-carboxylate (Intermediate 38, 1 g, 1.83 mmol, 1.0 eq) in THF (5 mL)/H₂O (5 mL) was added lithium hydroxide (0.08 mL, 8.22 mmol, 4.5 eq) at 20° C. under N₂, then the mixture was stirred at 30° C. for 16 h. The mixture was concentrated under reduced pressure. The residue was purified by reverse phase HPLC (H₂O (0.1% FA)-MeCN; 40-60% B) to afford the title compound (480.0 mg, 1.14 mmol, 63% yield). MS: m/z=420.3. [M+H]+, ESI pos.

### Intermediate 47

### ethyl 5-[bis(tert-butoxycarbonyl)amino]-1-[2,3-difluoro-4-(oxetan-3-yloxy)phenyl]imidazole-4-carboxylate

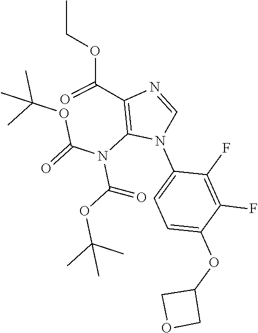 [SMILES: CCOC(=O)c1ncn(-c2ccc(OC3COC3)c(F)c2F)c1N(C(=O)OC(C)(C)C)C(=O)OC(C)(C)C]

### Step 1: 1-benzyloxy-2,3-difluoro-4-nitrobenzene

To a solution of 1,2,3-trifluoro-4-nitro benzene (25 g, 141.2 mmol, 1.0 eq) and K₂CO₃ (40.97 g, 296.48 mmol, 2.1 eq) in DMF (250 mL) was added benzyl alcohol (16.79 g, 155.3 mmol, 1.1 eq). The mixture was stirred at 10° C. for 16 h. The mixture was diluted with H₂O (500 ml) and extracted with EtOAc (3×200 mL). The combined organic phase was washed with brine (3×500 mL), dried over Na₂SO₄, filtered and concentrated under reduced pressure. The residue was purified by column (PE:EtOAc 1:0 to 5:1) to afford the title compound (9 g, 33.93 mmol, 24% yield). 1H NMR (400 MHz, DMSO-d₆) δ 8.13-8.04 (m, 1H), 7.51-7.30 (m, 6H), 5.38 (s, 2H) ppm.

### Step 2: 4-benzyloxy-2,3-difluoro-aniline

To a solution of 1-benzyloxy-2,3-difluoro-4-nitrobenzene (1 g, 3.77 mmol, 1.0 eq) and NH₄Cl (2 g, 37.7 mmol, 10.0 eq) in MeOH (50 mL) and H₂O (10 mL) was added Fe (2.11 g, 37.7 mmol, 10.0 eq). The mixture was stirred at 50° C. for 3 h. The mixture was filtered, and the filtrate was extracted with EtOAc (2×50 mL). The combined organic phase was washed with brine (100 mL), dried over Na₂SO₄, and filtered. The filtrate was concentrated under reduced pressure to afford the title compound (850 mg, 3.61 mmol, 92% yield). MS: m/z=236.0 [M+H]+, ESI pos.

### Step 3: ethyl 5-amino-1-(4-benzyloxy-2,3-difluorophenyl)imidazole-4-carboxylate

A mixture of ethyl 2-amino-2-cyanoacetate; 4-methylbenzene-1-sulphonic acid (1.36 g, 4.52 mmol, 1.25 eq), triethylamine (0.76 mL, 5.42 mmol, 1.5 eq) and trimethyl orthoformate (0.38 g, 3.61 mmol, 1.0 eq) was stirred at 90° C. for 4 h. The mixture was concentrated under reduced pressure. The residue was dissolved in MeCN (10 mL) and 4-benzyloxy-2,3-difluoro-aniline (0.85 g, 3.61 mmol, 1.0 eq) was added. The mixture was stirred at 90° C. for another 16 h. The reaction mixture was concentrated under reduced pressure and the residue was purified by reverse phase HPLC (H₂O (0.1% FA)-MeCN, 60-80% B) to afford the title compound (550 mg, 1.47 mmol, 34% yield). MS: m/z=374.2 [M+H]+, ESI pos.

### Step 4: ethyl 1-(4-benzyloxy-2,3-difluorophenyl)-5-[bis(tert-butoxycarbonyl)amino]imidazole-4-carboxylate

To a solution of ethyl 5-amino-1-(4-benzyloxy-2,3-difluorophenyl)imidazole-4-carboxylate (550 mg, 1.47 mmol, 1.0 eq), DMAP (0.54 g, 4.42 mmol, 3.0 eq) and triethylamine (0.82 mL, 5.89 mmol, 4.0 eq) in CH₂Cl₂ (10 mL) was added Boc₂O (0.71 g, 3.24 mmol, 2.2 eq), then the mixture was stirred at 25° C. for 2 h. The mixture was concentrated under reduced pressure. The residue was further purified by reverse phase HPLC (0.1% FA in H₂O-MeCN, 50-80% B) to afford the title compound (720 mg, 1.26 mmol, 85% yield). MS: m/z=574.3 [M+H]+, ESI pos.

### Step 5: ethyl 5-[bis(tert-butoxycarbonyl)amino]-1-(2,3-difluoro-4-hydroxyphenyl)imidazole-4-carboxylate

To a solution of ethyl 1-(4-benzyloxy-2,3-difluorophenyl)-5-[bis(tert-butoxycarbonyl)amino]imidazole-4-carboxylate (0.72 g, 1.26 mmol, 1.0 eq) in MeOH (10 mL) was added Pd/C (200 mg, 10%) under N₂. The mixture was then stirred at 20° C. for 16 h under H₂ (30 psi). The mixture was filtered and concentrated under reduced pressure afford the title compound (580 mg, 1.2 mmol, 92% yield) as light-yellow solid. MS: m/z=484.3. [M+H]+, ESI pos.

### Step 6: ethyl 5-[bis(tert-butoxycarbonyl)amino]-1-[2,3-difluoro-4-(oxetan-3-yloxy)phenyl]imidazole-4-carboxylate

To a mixture of ethyl 5-[bis(tert-butoxycarbonyl)amino]-1-(2,3-difluoro-4-hydroxyphenyl)imidazole-4-carboxylate (200 mg, 0.41 mmol, 1.0 eq) in DMF (8 mL) were added K₂CO₃ (172 mg, 1.24 mmol, 3.0 eq) and 3-iodooxetane (152 mg, 0.83 mmol, 2.0 eq). The mixture was then stirred at 90° C. for 16 h. The mixture was purified by reverse phase HPLC (0.1% FA in H₂O-MeCN, 50-80% B) to afford the title compound (140 mg, 0.26 mmol, 61% yield). MS: m/z=540.2 [M+H]+, ESI pos.

### Intermediate 48

### 5-amino-1-[2,3-difluoro-4-[1-(2-methoxyethyl)-3-methyl-pyrazol-4-yl]phenyl]imidazole-4-carboxylic acid

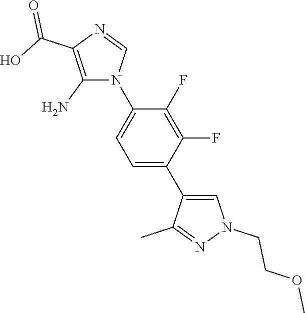 [SMILES: COCCn1cc(-c2ccc(-n3cnc(C(=O)O)c3N)c(F)c2F)c(C)n1]

### Step 1: 1-(2-methoxyethyl)-3-methyl-4-(4,4,5,5-tetramethyl-1,3,2-dioxaborolan-2-yl)pyrazole

To mixture of 5-methyl-4-(4,4,5,5-tetramethyl-1,3,2-dioxaborolan-2-yl)-1H-pyrazole (40 g, 192.24 mmol, 1.0 eq), K₂CO₃ (53.06 g, 384.49 mmol, 2.0 eq) in DMF (200 mL) was added 2-bromoethyl methyl ether (36.13 mL, 384.49 mmol, 2.0 eq). The mixture was stirred at 110° C. for 12 h. The reaction was concentrated to dryness and the residue was taken up in EtOAc (500 ml) and the organics washed with 2×500 ml water then 1×500 ml brine. The organics were then separated and dried (MgSO4) before concentration to dryness. The crude material was then purified by silica gel chromatography eluting 30% EtOAc in Isohexane. The desired fractions were concentrated to dryness in vacuo to afford the title compound as a mixture of regioisomers (33 g, 123.99 mmol, 58.05% yield) as colorless oil. MS: 267 [M+H]+

### Step 2: 2,3-difluoro-4-[1-(2-methoxyethyl)-3-methyl-pyrazol-4-yl]aniline

To a solution of 1-(2-methoxyethyl)-3-methyl-4-(4,4,5,5-tetramethyl-1,3,2-dioxaborolan-2-yl)pyrazole (mixture of regioisomers)(1407 mg, 5.29 mmol, 0.55 eq)/1-(2-methoxyethyl)-5-methyl-4-(4,4,5,5-tetramethyl-1,3,2-dioxaborolan-2-yl)pyrazole (2.8 g, 1.1 eq) in 1,4-Dioxane (80 mL) were added 4-bromo-2,3-difluoroaniline (2000 mg, 9.62 mmol, 1.0 eq), [1,1′-bis(diphenylphosphino)ferrocene]dichloropalladium(II)(703 mg, 0.96 mmol, 0.1 eq) and potassium carbonate (2658 mg, 19.23 mmol, 2.0 eq) under N₂ atmosphere. The resultant mixture was stirred at 110° C. for 15 h. The mixture was concentrated under vacuo, the residue diluted with EtOAc (50 mL) and washed with H₂O (25 mL×3), dried over Na₂SO₄ and concentrated under vacuo and the residue was purified by silica gel chromatography (PE:EA=65:35) to afford the title compound as mixture of regioisomers (3.58 g, 69% yield)) as a yellow oil. MS: 268.1 [M+H]+

### Step 3: ethyl 5-amino-1-[2,3-difluoro-4-[1-(2-methoxyethyl)-3-methyl-pyrazol-4-yl]phenyl]imidazole-4-carboxylate

To a solution of ethyl 2-amino-2-cyanoacetate; 4-methylbenzene-1-sulfonic aci (3.02 g, 10.05 mmol, 3.0 eq) were added triethylamine (2.8 mL, 20.09 mmol, 6.0 eq) and triethyl orthoformate (4.96 g, 33.49 mmol, 10.0 eq), the resultant mixture was stirred at 80° C. for 2 h. The solvent was removed under vacuo and the residue was diluted with ACN (90 mL), then 2,3-difluoro-4-[1-(2-methoxyethyl)-3-methyl-pyrazol-4-yl]aniline as a mixture of regioisomers (1.79 g, 3.35 mmol, 1.0 eq) was added and the resultant mixture was stirred at 40° C. for 15 h. The mixture was concentrated under vacuum and the residue was purified by silica gel chromatography on silica gel to afford the title compound ethyl 5-amino-1-[2,3-difluoro-4-[1-(2-methoxyethyl)-3-methyl-pyrazol-4-yl]phenyl]imidazole-4-carboxylate (840 mg, 2.07 mmol, 61.88% yield) as off white solid and the corresponding regioisomer ethyl 5-amino-1-[2,3-difluoro-4-[1-(2-methoxyethyl)-5-methyl-pyrazol-4-yl]phenyl]imidazole-4-carboxylate (430 mg, 1.06 mmol, 31.68% yield) as off-white solid. MS: 406.1 [M+H]+

### Step 4: 5-amino-1-[2,3-difluoro-4-[1-(2-methoxyethyl)-3-methyl-pyrazol-4-yl]phenyl]imidazole-4-carboxylic acid

To a solution of ethyl 5-amino-1-[2,3-difluoro-4-[1-(2-methoxyethyl)-3-methyl-pyrazol-4-yl]phenyl]imidazole-4-carboxylate (820 mg, 2.02 mmol, 1.0 eq), sodium hydroxide (404 mg, 10.11 mmol, 5.0 eq) in IPA (10 mL) and water (3 mL) was stirred at 70° C. for 4 h. The residue was diluted with 100 ml of water, then adjusted to pH 6˜7 with HCl (2M) and extracted with 3×10⁰ mL of EtOAc. The organic layers were combined and concentrated under vacuum to afford the title compound (640 mg, 1.7 mmol, 77.98% yield). MS: 378.1 [M+H]+

### Intermediate 49

### 5-[(E)-dimethylaminomethyleneamino]-1-(4-fluoro-1-methyl-indazol-5-yl)imidazole-4-carbonyl chloride

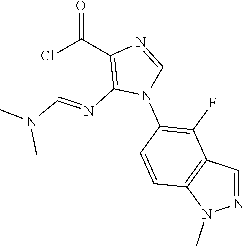 [SMILES: CN(C)/C=N/c1c(C(=O)Cl)ncn1-c1ccc2c(cnn2C)c1F]

### Step 1: 5-bromo-4-fluoro-1-methyl-indazole

iodomethane (11882 mg, 83.71 mmol, 3.0 eq) was added to a mixture of 5-bromo-4-fluoro-1H-indazole ([1082041-85-7], 6000 mg, 27.9 mmol, 1.0 eq) and potassium tert-butoxide (6262 mg, 55.81 mmol, 2.0 eq) in THF (100 mL), the mixture was stirred at 70° C. for 2 h. The mixture was diluted with EtOAc (20 mL), washed with NH₄Cl (aq, 20 mL) and water (20 mL), dried over anhydrous Na₂SO4, filtered, concentrated and purified by flash column chromatography eluting 0˜20% EtOAc in PE to afford the title compound (3200 mg, 13.97 mmol, 50.07% yield) along with the regiosiomer 5-bromo-4-fluoro-2-methyl-indazole (1000 mg, 4.37 mmol, 15.65% yield). MS: 229.0, 231.0 [M+H]+

### Step 2: N-(4-fluoro-1-methyl-indazol-5-yl)-1,1-diphenyl-methanimine

A mixture of 5-bromo-4-fluoro-1-methyl-indazole (3000 mg, 13.1 mmol, 1.0 eq), [1,3-Bis(2,6-Di-3-pentylphenyl) imidazol-2-ylidene](3-chloropyridyl)dichloropalladium(II)(518 mg, 0.65 mmol, 0.05 eq), cesium carbonate (12802 mg, 39.29 mmol, 3.0 eq) and diphenylmethanimine (2.2 mL, 13.1 mmol, 1.0 eq) in 1,4-dioxane (60 mL) was heated to 100° C. and stirred for 10 h under nitrogen atmosphere. The reaction was filtered, the filtrate was concentrated and purified by flash column chromatography eluting 0˜15% EtOAc in PE to the title compound (1.6 g, 4.86 mmol, 34.3% yield). MS: 330.1 [M+H]

### Step 3: 4-fluoro-1-methyl-indazol-5-amine

36% hydrochloric acid (4919 mg, 48.58 mmol, 10.0 eq) was added dropwise to a solution of N-(4-fluoro-1-methyl-indazol-5-yl)-1,1-diphenyl-methanimine (1600 mg, 4.86 mmol, 1.0 eq) in THF (40 mL). The mixture was stirred at 25° C. for 0.5 h. The mixture was filtered, the filter cake was dissolved in water (20 mL), adjust pH to 8 with NaHCO₃(aq), then extracted with EtOAcA (20 mL*2), the organics was washed with brine (40 mL), dried over anhydrous Na₂SO₄, filtered and concentrated in vacuum to afford the title compound (600 mg, 3.63 mmol, 74.78% yield). MS: 166.1 [M+H]+

### Step 4: ethyl 5-amino-1-(4-fluoro-1-methyl-indazol-5-yl)imidazole-4-carboxylate

A mixture of ethyl 2-amino-2-cyanoacetate; 4-methylbenzene-1-sulfonic acid (2181 mg, 7.27 mmol, 2.0 eq), triethylamine (2.03 mL, 14.53 mmol, 4.0 eq) and triethyl orthoformate (26917 mg, 181.63 mmol, 50.0 eq) was heated to 80° C. and stirred for 2 h, the solvent was removed under vacuo and the residue was diluted with ACN (10 mL). Then 4-fluoro-1-methyl-indazol-5-amine (600 mg, 3.63 mmol, 1.0 eq) was added and the resultant mixture was stirred at 40° C. for 15 h. The mixture was concentrated and purified by flash column chromatography eluting 0˜10% MeOH in DCM to afford the title compound (420 mg, 1.38 mmol, 38.12% yield). MS: 304.1 [M+H]+

### Step 5: 5-amino-1-(4-fluoro-1-methyl-indazol-5-yl)imidazole-4-carboxylic acid

A solution of sodium hydroxide (263 mg, 6.59 mmol, 5.0 eq) in water (5 mL) was added to a mixture of ethyl 5-amino-1-(4-fluoro-1-methyl-indazol-5-yl)imidazole-4-carboxylate (400 mg, 1.32 mmol, 1.0 eq) in IPA (10 mL), the mixture was heated to 50° C. and stirred for 6 h. The reaction was concentrated to dryness, then diluted with water (20 mL), adjust pH to 5˜6 with HCl (1M), filtered, the filter cake was dried in vacuum to afford the title compound (300 mg, 1.09 mmol, 82.65% yield). MS: 276.1 [M+H]+

### Step 6: 5-[(E)-dimethylaminomethyleneamino]-1-(4-fluoro-1-methyl-indazol-5-yl)imidazole-4-carbonyl chloride

Oxalyl chloride (92 mg, 0.73 mmol, 2.0 eq) was added to a mixture of 5-amino-1-(4-fluoro-1-methyl-indazol-5-yl)imidazole-4-carboxylic acid (100 mg, 0.36 mmol, 1.0 eq) and N, N-dimethylformamide (13 mg, 0.18 mmol, 0.5 eq) in DCM (5 mL) dropwise, the mixture was stirred at 25° C. for 2 h. The mixture was concentrated in vacuum to afford the title compound (120 mg, 0.34 mmol, 33.57% yield), which was used without further purification. MS: 345.2 [M−Cl+OMe+H]+, (quenched with MeOH)

### Intermediate 50

### ethyl 5-amino-1-(4-fluoro-1-methyl-indol-5-yl)imidazole-4-carboxylate

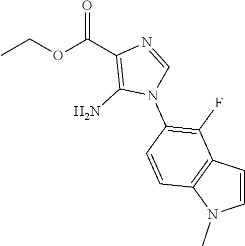 [SMILES: CCOC(=O)c1ncn(-c2ccc3c(ccn3C)c2F)c1N]

### Step 1: 4-fluoro-1-methyl-5-nitro-indole

A mixture of 4-fluoro-5-nitro-1H-indole (1000 mg, 5.55 mmol, 1.0 eq), potassium carbonate (2.3 g, 16.7 mmol, 3 eq) and iodomethane (0.52 mL, 8.33 mmol, 1.5 eq) in ACN (5 mL) was stirred at 25° C. for 4 h. The reaction was concentrated to dryness and the residue was taken up in EtOAc (3 ml) and the organics washed with 2×3 ml water then 1×3 ml saturated brine solution. The organics were then separated and dried (Na2SO4) before concentration to dryness. The crude was then purified by flash column chromatography eluting 20% EtOAc in PE. The desired fractions were concentrated to dryness in vacuo to get the title compound (570 mg, 2.94 mmol, 52.88% yield). MS: 195.1 [M+H]+

### Step 2: 4-fluoro-1-methyl-indol-5-amine

A solution of 4-fluoro-1-methyl-5-nitro-indole (570 mg, 2.94 mmol, 1.0 eq), iron (820 mg, 14.68 mmol, 5.0 eq) and ammonium chloride (785 mg, 14.68 mmol, 5.0 eq) in ethanol (5 mL) and water (1 mL) was stirred at 50° C. for 2 h. The solid was filtered. The crude was then purified by flash column chromatography eluting 40% EtOAc in PE. The desired fractions were concentrated to dryness in vacuo to afford the title compound (400 mg, 2.44 mmol, 82.99% yield).

MS: 165.1 [M+H]+

### Step 3: ethyl 5-amino-1-(4-fluoro-1-methyl-indol-5-yl)imidazole-4-carboxylate

To a solution of ethyl 2-amino-2-cyanoacetate; 4-methylbenzene-1-sulfonic acid (1463 mg, 4.87 mmol, 2.0 eq) in ACN (2 mL) were added triethylamine (1.36 mL, 9.75 mmol, 4.0 eq) and triethyl orthoformate (10.73 mL, 64.48 mmol, 26.47 eq), the resultant mixture was stirred at 80° C. for 2 h, the solvent was removed under vacuo and the residue was diluted with ACN (2 mL), then 4-fluoro-1-methyl-indol-5-amine (400 mg, 2.44 mmol, 1.0 eq) was added and the resultant mixture was stirred at 50° C. for 15 h. The mixture was concentrated under vacuum and the residue was purified by silica gel chromatography (PE:EA=30:70) to afford the title compound (550 mg, 1.82 mmol, 74.67% yield). MS: 303.2 [M+H]+

### Intermediate 51

### 5-amino-1-[1-(2-fluoroallyl)-3-(trifluoromethyl)pyrazol-4-yl]imidazole-4-carboxylic acid

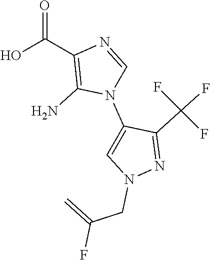 [SMILES: C=C(F)Cn1cc(-n2cnc(C(=O)O)c2N)c(C(F)(F)F)n1]

### Step 1: 2-fluoroallyl 4-methylbenzenesulfonate

A solution of 2-fluoroprop-2-en-1-ol ([5675-31-0], 10 g, 131.46 mmol, 1.0 eq) in DCM (100 mL) was added p-toluenesulfonyl chloride (37.59 g, 197.19 mmol, 1.5 eq), triethylamine (55 mL, 394.37 mmol, 3.0 eq) and 4-dimethylaminopyridine (1.61 g, 13.15 mmol, 0.1 eq) the reaction was stirred at 25° C. for 4 h The reaction was concentrated to dryness. The crude material was purified by silica gel chromatography eluting 10% EtOAc in Isohexane. The desired fractions were concentrated to dryness in vacuo to afford the title compound (13 g, 56.46 mmol, 41.28% yield) as colorless oil. MS: 231.0 [M+H]+

### Step 2: 1-(2-fluoroallyl)-4-nitro-3-(trifluoromethyl)pyrazole

A solution of 4-nitro-3-(trifluoromethyl)-1H-pyrazole (10.22 g, 56.46 mmol, 1.0 eq) in DMF (80 mL) was added 2-fluoroallyl 4-methylbenzenesulfonate (13 g, 56.46 mmol, 1.0 eq) and potassium carbonate (23.41 g, 169.37 mmol, 3.0 eq) the reaction was stirred at 25° C. for 16 h The reaction was concentrated to dryness and the residue diluted with EtOAc (20 ml) and the organic layer washed with 2×20 ml water then 1×20 ml brine. The organic layer was dried (MgSO4) before concentration to dryness. The crude material was then purified by silica gel chromatography eluting 60% EtOAc in Isohexane. The desired fractions were concentrated to dryness in vacuo to afford the title compound (11.4 g, 47.67 mmol, 71.36% yield) as yellow oil. MS: 240.1 [M+H]+

### Step 3: 1-(2-fluoroallyl)-3-(trifluoromethyl)pyrazol-4-amine

To a solution of 1-(2-fluoroallyl)-4-nitro-3-(trifluoromethyl)pyrazole (11.42 g, 47.76 mmol, 1.0 eq) in ethanol (100 mL) were added iron powder (10.67 g, 191.03 mmol, 4.0 eq), ammonium chloride (20.44 g, 382.05 mmol, 8.0 eq) and water (30 mL) under N₂ atmosphere. The resultant mixture was stirred at 50° C. for 2 h. The solid was filtered, and the filtrate was diluted with EtOAc (80 mL) and washed with water (25 mL×3), dried over Na₂SO₄ and evaporated under reduced pressure to afford the title compound (9678 mg, 46.28 mmol, 85.27% yield) as a light yellow oil, which was used without further purification. MS: 210.1 [M+H]+

### Step 4: ethyl 5-amino-1-[1-(2-fluoroallyl)-3-(trifluoromethyl)pyrazol-4-yl]imidazole-4-carboxylate

To a mixture of of ethyl 2-amino-2-cyanoacetate, 4-methylbenzene-1-sulfonic aci (27.8 g, 92.55 mmol, 2.0 eq) in triethyl orthoformate (170 mL, 1016.1 mmol, 21.96 eq) was added triethylamine (18.73 g, 185.1 mmol, 4.0 eq), the resultant mixture was stirred at 82° C. for 2 h. The solvent was removed under vacuo and the residue was diluted with ACN (120 mL). Then 1-(2-fluoroallyl)-3-(trifluoromethyl)pyrazol-4-amine (9.68 g, 46.28 mmol, 1.0 eq) was added and the resultant mixture was stirred at 25° C. for 15 h. The reaction was concentrated to dryness and the residue was diluted with EtOAc (100 mL) and the organics washed with 3×30 mL water then 1×30 mL brine. The layer was dried (Na2SO4) before concentration to dryness. The crude material was then purified by silica gel chromatography eluting 3% MeOH in DCM. The desired fractions were concentrated to dryness in vacuo to afford the title compound (7800 mg, 22.46 mmol, 46.11% yield) as a light yellow solid. MS: 348.1 [M+H]+

### Step 5: 5-amino-1-[1-(2-fluoroallyl)-3-(trifluoromethyl)pyrazol-4-yl]imidazole-4-carboxylic acid

To a solution of ethyl 5-amino-1-[1-(2-fluoroallyl)-3-(trifluoromethyl)pyrazol-4-yl]imidazole-4-carboxylate (3300 mg, 9.5 mmol, 1.0 eq) in IPA (50 mL) was added lithium hydroxide hydrate (saturated aqueous solution) (10 mL, 55.0 mmol, 5.79 eq) under N2, the resultant mixture was stirred at 35° C. for 48 h. The solvent was removed under vacuo, then diluted with H2O (50 mL), extracted with EtOAc (40 mL×3) and the organic layers were discarded. The aqueous layer was acidified with 4M HCl to pH to 5˜6 and the resulting solid filtered and dried to afford the title compound (950 mg, 2.98 mmol, 18.79% yield), which was used without further purification. MS: 320.0 [M+H]+

### Intermediate 52

### [4-[(3aS,6aS)-2-methyl-1,3,3a,4,6,6a-hexahydropyrrolo[3,4-c]pyrrole-5-carbonyl]piperazin-1-yl]-(4-amino-2-fluorophenyl)methanone

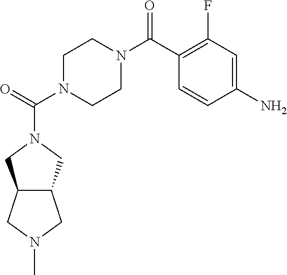 [SMILES: CN1C[C@H]2CN(C(=O)N3CCN(C(=O)c4ccc(N)cc4F)CC3)C[C@@H]2C1]

### Step 1: tert-butyl (3aR,6aR)-2-[4-(2-fluoro-4-nitrobenzoyl)piperazine-1-carbonyl]-1,3,3a,4,6,6a-hexahydropyrrolo[3,4-c]pyrrole-5-carboxylate

A mixture (2-fluoro-4-nitrophenyl)-piperazin-1-yl-methanone;hydrochloride ([1593114-33-0] 341 mg, 1.18 mmol, 1.0 eq), triethylamine (0.33 mL, 2.36 mmol, 2.0 eq) and bis(trichloromethyl) carbonate (350 mg, 1.18 mmol, 1.0 eq) in CH₂Cl₂ (5 mL) was stirred at 0° C. for 0.5 h, then tert-butyl (3aR,6aR)-2,3,3a,4,6,6a-hexahydro-1H-pyrrolo[3,4-c]pyrrole-5-carboxylate (250 mg, 1.18 mmol, 1.0 eq) was added. The resulting mixture was then stirred at 0° C. for 1 h. The reaction mixture was poured into H₂O (10 mL) and extracted with EtOAc (3×10 mL). The combined organic phase was washed with brine (2×10 mL), dried over Na₂SO₄, filtered and concentrated under reduced pressure. The residue was purified by reverse phase HPLC (H₂O-CAN; 40-50% B) to afford the title compound (300 mg, 0.61 mmol, 52% yield). MS: m/z=492.2 [M+H]+, ESI pos.

### Step 2: [4-[(3aS,6aS)-2,3,3a,4,6,6a-hexahydro-1H-pyrrolo[3,4-c]pyrrole-5-carbonyl]piperazin-1-yl]-(2-fluoro-4-nitrophenyl)methanone;2,2,2-trifluoroacetic acid

To a solution of tert-butyl (3aR,6aR)-2-[4-(2-fluoro-4-nitrobenzoyl)piperazine-1-carbonyl]-1,3,3a,4,6,6a-hexahydropyrrolo[3,4-c]pyrrole-5-carboxylate (300 mg, 0.61 mmol, 1.0 eq) in CH₂Cl₂ (5 mL) was added TFA (2.5 mL). The resulting solution was then stirred at 25° C. for 1.5 h. The solution was concentrated under reduced pressure, the residue was purified by reverse phase-HPLC (H₂O (0.1% FA-CAN; 20-50% B) to afford the title compound (300 mg, 0.59 mmol, 97% yield). MS: m/z=392.1 [M+H]+, ESI pos.

### Step 3: [4-[(3aS,6aS)-2-methyl-1,3,3a,4,6,6a-hexahydropyrrolo[3,4-c]pyrrole-5-carbonyl]piperazin-1-yl]-(2-fluoro-4-nitrophenyl)methanone

To a solution of [4-[(3aS,6aS)-2,3,3a,4,6,6a-hexahydro-1H-pyrrolo[3,4-c]pyrrole-5-carbonyl]piperazin-1-yl]-(2-fluoro-4-nitrophenyl)methanone;2,2,2-trifluoroacetic acid (300 mg, 0.59 mmol, 1.0 eq) in MeOH (5 mL) was added acetic acid (0.1 mL, 0.06 mmol, 0.1 eq) and formaldehyde (1.5 mL, 55.5 mmol, 93.5 eq) followed by sodium triacetoxyborohydride (188.7 mg, 0.89 mmol, 1.5 eq). The resulting mixture was then stirred at 20° C. for 16 h. The mixture was concentrated under reduced pressure, the residue was diluted with EtOAc (20 mL) and saturated NaHCO₃ solution (20 mL), the organic phase was separated and washed with brine (20 mL), dried over Na₂SO₄, concentrated under reduced pressure. The residue was purified by reverse phase HPLC (H₂O (0.1% FA)-CAN; 10-30% B) to afford the title compound (300 mg, 0.74 mmol, 96% yield). MS: m/z=406.1 [M+H]+, ESI pos.

### Step 4: [4-[(3aS,6aS)-2-methyl-1,3,3a,4,6,6a-hexahydropyrrolo[3,4-c]pyrrole-5-carbonyl]piperazin-1-yl]-(4-amino-2-fluorophenyl)methanone

To a mixture of [4-[(3aS,6aS)-2-methyl-1,3,3a,4,6,6a-hexahydropyrrolo[3,4-c]pyrrole-5-carbonyl]piperazin-1-yl]-(2-fluoro-4-nitrophenyl)methanone (300.0 mg, 0.74 mmol, 1.0 eq), NH₄Cl (316.7 mg, 5.92 mmol, 8.0 eq) in EtOH (8 mL) and H₂O (4 mL) was added iron (206 mg, 3.7 mmol, 5.0 eq). The resulting mixture was then stirred at 60° C. for 1.5 h. The mixture was filtered, and the filtrate was concentrated under reduced pressure. The residue was purified by reverse phase HPLC (H₂O (0.1% FA)-CAN; 40-60% B) to give the title compound (75 mg, 0.2 mmol, 27% yield). MS: m/z=376.2 [M+H]+, ESI pos.

The following compounds were prepared in analogy to the procedure above

| | | | | MS |
|---|---|---|---|---|
| Int | Structure | Name | Reagents | [M + H]+ |
| 53 | 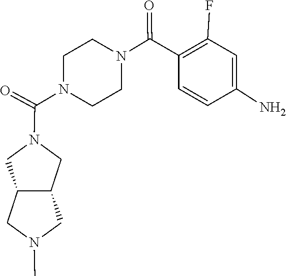 [SMILES: CN1C[C@@H]2CN(C(=O)N3CCN(C(=O)c4ccc(N)cc4F)CC3)C[C@@H]2C1] | [4-[(3aS,6aR)-2- methyl- 1,3,3a,4,6,6a- hexahydropyrrolo [3,4-c]pyrrole-5- carbonyl]piperazin- 1-yl]-(4-amino-2- fluorophenyl) methanone | (2-fluoro-4-nitrophenyl)- piperazin-1-yl- methanone;hydrochloride (3aR,6aS)-5-methyl- 2,3,3a,4,6,6a-hexahydro- 1H-pyrrolo[3,4-c]pyrrole [172739-03-6] | 376.1 |
| 54 | 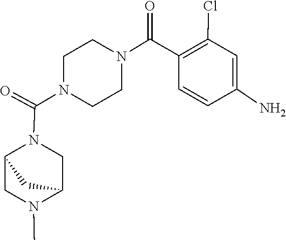 | (4-amino-2- chlorophenyl)-[4- [(1S,4S)-5-methyl- 2,5- diazabicyclo[2.2.1] heptane-2- carbonyl]piperazin- 1-yl]methanone | (2-chloro-4-nitrophenyl)- piperazin-1-yl- methanone;hydrochloride, (1S,4S)-2-BOC-2,5- diazabicyclo[2.2.1]heptane | 378.2 |
| 55 | 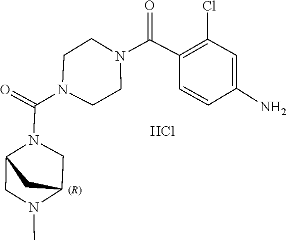 | (4-amino-2- chlorophenyl)-[4- [(1R,4R)-5-methyl- 2,5- diazabicyclo[2.2.1] heptane-2- carbonyl]piperazin- 1- yl]methanone; hydrochloride | (2-chloro-4-nitrophenyl)- piperazin-1-yl- methanone;hydrochloride, tert-butyl(1R,4R)-2,5- diazabicyclo[2.2.1]heptane- 2-carboxylate | 378.0 |

### Intermediate 56

### tert-butyl (3aR,5s,6aS)-5-(4-(4-amino-2-chlorobenzoyl)piperazine-1-carbonyl)hexahydrocyclopenta[c]pyrrole-2 (1H)-carboxylate

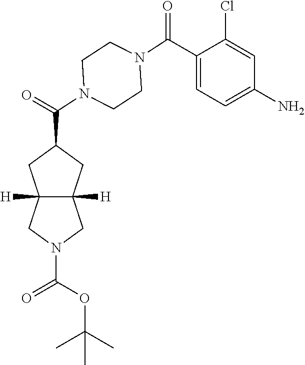 [SMILES: CC(C)(C)OC(=O)N1C[C@H]2C[C@H](C(=O)N3CCN(C(=O)c4ccc(N)cc4Cl)CC3)C[C@H]2C1]

### Step 1: tert-butyl (3aR,6aS)-5-[4-(2-chloro-4-nitro-benzoyl)piperazine-1-carbonyl]-3,3a,4,5,6,6a-hexahydro-1H-cyclopenta[c]pyrrole-2-carboxylate

To a solution of (2-chloro-4-nitro-phenyl)-piperazin-1-yl-methanone;hydrochloride ([1605138-92-8], 120 mg, 0.39 mmol, 1.0 eq) in DCM (2 mL) were added (3aR,6aS)-2-tert-butoxycarbonyl-3,3a,4,5,6,6a-hexahydro-1H-cyclopenta[c]pyrrole-5-carboxylic acid ([1401464-09-2], 100 mg, 0.39 mmol, 1.0 eq), O-(7-azabenzotriazol-1-yl)-N,N,N′,N′-tetramethyluronium hexafluorophosphate (193 mg, 0.51 mmol, 1.3 eq) and diisopropylethylamine (101 mg, 0.78 mmol, 2.0 eq), the resultant mixture was stirred at 25° C. for 3 h. The solvent was removed under vacuo, the residue was diluted with EtOAc (20 mL) and washed with H₂O (10 mL×3), dried over Na2SO4 and concentrated under vacuo. The residue was purified by silica gel chromatography (PE:EA=25:75) to afford the title compound (177 mg, 0.35 mmol, 83.73% yield) as a light yellow solid. MS: 450.9 [M−Bu+H]+

### Step 2: tert-butyl (3aR,6aS)-5-[4-(4-amino-2-chloro-benzoyl)piperazine-1-carbonyl]-3,3a,4,5,6,6a-hexahydro-1H-cyclopenta[c]pyrrole-2-carboxylate

To a solution of tert-butyl (3aR,6aS)-5-[4-(2-chloro-4-nitro-benzoyl)piperazine-1-carbonyl]-3,3a,4,5,6,6a-hexahydro-1H-cyclopenta[c]pyrrole-2-carboxylate (177 mg, 0.35 mmol, 1.0 eq) in ethanol (3 mL) were added iron atom (97 mg, 1.75 mmol, 5.0 eq), ammonium chloride (186 mg, 3.49 mmol, 10.0 eq) and water (1 mL) under N₂ atmosphere. The resultant mixture was stirred at 50° C. for 3 h. The solid was filtered and the filtrate concentrated under vacuo. The residue was diluted with EtOAc (20 mL) and washed with H₂O (15 mL×3), dried over Na₂SO₄ and concentrated under vacuo. The residue was purified by silica gel chromatography (PE:EA=70:30) to afford the title compound (150 mg, 0.31 mmol, 83.77% yield). MS: 421.0 [M−Bu+H]+

### Intermediate 57

### (4-amino-2-chlorophenyl)-[2-[(1R,5S,6r)-3-methyl-3-azabicyclo[3.1.0]hexane-6-carbonyl]-2,6-diazaspiro[3.3]heptan-6-yl]methanone;hydrochloride

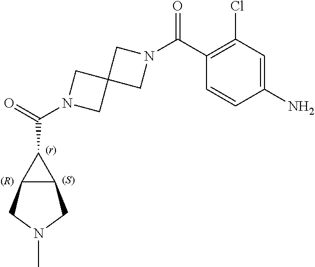

### Step 1: tert-butyl 6-(2-chloro-4-nitrobenzoyl)-2,6-diazaspiro[3.3]heptane-2-carboxylate

To a mixture of 2-chloro-4-nitrobenzoic acid (1.02 g, 5.04 mmol, 1.0 eq), tert-butyl 2,6-diazaspiro[3.3]heptane-2-carboxylate (1 g, 5.04 mmol, 1.0 eq) and N,N-diisopropylethylamine (2.64 mL, 15.13 mmol, 3.0 eq) in DMF (10 mL) was added HATU (1.31 g, 5.55 mmol, 1.1 eq). The mixture was then stirred at 10° C. for 2 h. The mixture was diluted with H₂O (80 mL) and extracted with EtOAc (3×50 mL). The combined organic phase was washed with brine (3×50 mL), dried over Na₂SO₄ and concentrated under reduced pressure. The residue was purified by column chromatography (SiO₂, PE:EtOAc 2:1 to 1:2) to afford the title compound (1 g, 2.62 mmol, 49% yield) as a yellow oil. MS: m/z=382.1 [M+H]+, ESI pos.

### Step 2: (2-chloro-4-nitrophenyl)-(2,6-diazaspiro[3.3]heptan-2-yl)methanone; 2,2,2-trifluoroacetic acid

To a solution of tert-butyl 6-(2-chloro-4-nitrobenzoyl)-2,6-diazaspiro[3.3]heptane-2-carboxylate (1 g, 2.62 mmol, 1.0 eq) in CH₂Cl₂ (10 mL) was added TFA (5.0 mL, 64.9 mmol, 24.78 eq). The mixture was then stirred at 10° C. for 0.5 h. The mixture was concentrated under reduced pressure to afford the title compound (810 mg, 2.05 mmol, 77% yield) as yellow oil.

MS: m/z=282.0 [M+H]+, ESI pos.

### Step 3: tert-butyl (1R,5S,6r)-6-[6-(2-chloro-4-nitrobenzoyl)-2,6-diazaspiro[3.3]heptane-2-carbonyl]-3-azabicyclo[3.1.0]hexane-3-carboxylate

To a mixture of (2-chloro-4-nitrophenyl)-(2,6-diazaspiro[3.3]heptan-2-yl)methanone;2,2,2-trifluoroacetic acid (398 mg, 1.01 mmol, 1.0 eq), (1R,5S,6r)-3-tert-butoxycarbonyl-3-azabicyclo[3.1.0]hexane-6-carboxylic acid (229 mg, 1.01 mmol, 1.0 eq) and N,N-diisopropylethylamine (0.53 mL, 3.02 mmol, 3.0 eq) in DMF (5 mL) was added HATU (260 mg, 1.11 mmol, 1.1 eq). The mixture was stirred at 10° C. for 1 h. The mixture was purified by reverse phase HPLC ([H₂O (0.1% FA)-MeCN]; 55-60% B) to afford the title compound (400 mg, 0.81 mmol, 74% yield). MS: m/z=491.3 [M+H]+, ESI pos.

### Step 4: [2-[(1R,5S,6r)-3-azabicyclo[3.1.0]hexane-6-carbonyl]-2,6-diazaspiro[3.3]heptan-6-yl]-(2-chloro-4-nitrophenyl)methanone;2,2,2-trifluoroacetic acid

To a mixture of tert-butyl (1R,5S)-6-[6-(2-chloro-4-nitrobenzoyl)-2,6-diazaspiro[3.3]heptane-2-carbonyl]-3-azabicyclo[3.1.0]hexane-3-carboxylate (400 mg, 0.81 mmol, 1.0 eq) in CH₂Cl₂ (6 mL) was added TFA (1.5 mL, 19.5 mmol, 24 eq). The mixture was stirred at 10° C. for 1 h. The mixture was concentrated to afford the title compound (300 mg, 0.59 mmol, 68% yield) as yellow oil. MS: m/z=391.0 [M+H]+, ESI pos.

### Step 5: (2-chloro-4-nitrophenyl)-[2-[(1R,5S,6r)-3-methyl-3-azabicyclo[3.1.0]hexane-6-carbonyl]-2,6-diazaspiro[3.3]heptan-6-yl]methanone

To a solution of [2-[(1R,5S)-3-azabicyclo[3.1.0]hexane-6-carbonyl]-2,6-diazaspiro[3.3]heptan-6-yl]-(2-chloro-4-nitrophenyl)methanone;2,2,2-trifluoroacetic acid (300 mg, 0.59 mmol, 1.0 eq) in MeOH (5 mL) was added AcOH (3 mg, 0.06 mmol, 0.1 eq) and formaldehyde (48 mg, 0.59 mmol, 1.0 eq) followed by sodium triacetoxyborohydride (188 mg, 0.89 mmol, 1.5 eq), the mixture was stirred at 10° C. for 2 h. The reaction mixture was concentrated under reduced pressure, the residue was purified by reverse-phase HPLC (H₂O (0.1% FA)-MeCN; 40-45% B) to afford the title compound (230.0 mg, 0.57 mmol, 95% yield). MS: m/z=405.0 [M+H]+, ESI pos.

### Step 6: (4-amino-2-chlorophenyl)-[2-[(1R,5S,6r)-3-methyl-3-azabicyclo[3.1.0]hexane-6-carbonyl]-2,6-diazaspiro[3.3]heptan-6-yl]methanone;hydrochloride

To a solution of (2-chloro-4-nitrophenyl)-[2-[(1R,5S)-3-methyl-3-azabicyclo[3.1.0]hexane-6-carbonyl]-2,6-diazaspiro[3.3]heptan-6-yl]methanone (230 mg, 0.57 mmol, 1.0 eq) in EtOH (3 mL) and H₂O (0.5 mL) was added NH₄Cl (243 mg, 4.54 mmol, 8.0 eq) and iron (158 mg, 2.84 mmol, 5.0 eq). The mixture was then stirred at 50° C. for 2 h. The mixture was filtered and washed with EtOH). The filtrate was concentrated under reduced pressure. The residue was purified by reverse phase HPLC (H₂O (0.1% HCl)-MeCN; 10-20% B) to afford the title compound (150 mg, 0.36 mmol, 69% yield).

MS: m/z=375.2 [M+H]+, ESI pos.

### Intermediate 58

### tert-butyl 4-[4-(4-amino-2-chloro-benzoyl)piperazine-1-carbonyl]-4-fluoro-piperidine-1-carboxylate

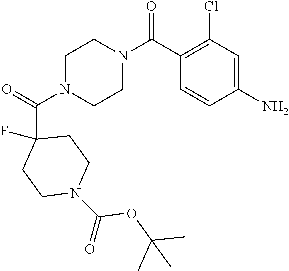 [SMILES: CC(C)(C)OC(=O)N1CCC(F)(C(=O)N2CCN(C(=O)c3ccc(N)cc3Cl)CC2)CC1]

To a solution of 1-BOC-4-fluoro-4-piperidinecarboxylic acid (286 mg, 1.16 mmol, 0.8 eq), (4-amino-2-chloro-phenyl)-piperazin-1-yl-methanone;hydrochloride (400 mg, 1.4 mmol, 1 eq) and N,N-diisopropylethylamine (0.76 mL, 4.35 mmol, 3.0 eq) in DMF (8 mL) was added CMPI ([14338-32-0], 296 mg, 1.16 mmol, 0.8 eq). The mixture was stirred at 15° C. for 1 h. The mixture was purified by reverse-phase HPLC (H₂O (0.1% HCl)-MeCN]; 60-65% B) to afford the title compound (300 mg, 0.64 mmol, 40% yield) as yellow oil.

MS: m/z=491.2 [M+Na]+, ESI pos.

### Intermediate 59

### tert-butyl (3aR,6aS)-2-[1-(4-amino-2-chloro-benzoyl)piperidine-4-carbonyl]-1,3,3a,4,6,6a-hexahydropyrrolo[3,4-c]pyrrole-5-carboxylate

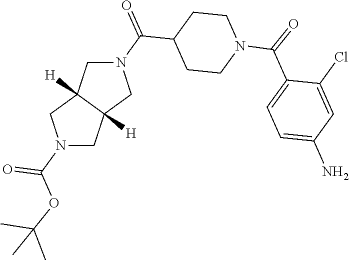 [SMILES: CC(C)(C)OC(=O)N1C[C@@H]2CN(C(=O)C3CCN(C(=O)c4ccc(N)cc4Cl)CC3)C[C@@H]2C1]

### Step 1: tert-butyl (3aR,6aS)-2-[1-(2-chloro-4-nitro-benzoyl)piperidine-4-carbonyl]-1,3,3a,4,6,6a-hexahydropyrrolo[3,4-c]pyrrole-5-carboxylate

To a solution of 1-(2-chloro-4-nitro-benzoyl)piperidine-4-carboxylic acid ([1054138-74-7], 2500 mg, 7.99 mmol, 1.0 eq) and 1,1-Dimethylethyl (3aR,6aS)-hexahydropyrrolo[3,4-c]pyrrole-2 (1H)-carboxylate ([250275-15-1], 1697 mg, 7.99 mmol, 1.0 eq) in DCM (25 mL) was added 2-(7-azabenzotriazol-1-yl)-N,N,N′,N′-tetramethyluronium hexafluorophosphate (6080 mg, 15.99 mmol, 2.0 eq) and N,N-diisopropylethylamine (4.18 mL, 23.98 mmol, 3.0 eq) and the mixture stirred at 25° C. for 2 h. The reaction was quenched with water (25 mL). The resulting solution was extracted with ethyl acetate (20 mL×3). The combined organic phases were dried over anhydrous sodium sulfate and concentrated under reduced pressure. The residue was purified by flash column to afford the title compound (3300 mg, 6.51 mmol, 81.42% yield) as a yellow oil.

MS: 407.0 [M+H-Boc]+

### Step 2: tert-butyl (3aR,6aS)-2-[1-(4-amino-2-chloro-benzoyl)piperidine-4-carbonyl]-1,3,3a,4,6,6a-hexahydropyrrolo[3,4-c]pyrrole-5-carboxylate

A solution of tert-butyl (3aR,6aS)-2-[1-(2-chloro-4-nitro-benzoyl)piperidine-4-carbonyl]-1,3,3a,4,6,6a-hexahydropyrrolo[3,4-c]pyrrole-5-carboxylate (4000 mg, 7.89 mmol, 1.0 eq), iron (2203 mg, 39.45 mmol, 5.0 eq) and ammonium chloride (2110 mg, 39.45 mmol, 5.0 eq) in ethanol (30 mL) and water (6 mL) was stirred at 50° C. for 2 h. Then the suspension was filtered. The crude material was then purified by flash column chromatography eluting 40% EtOAc in PE. The desired fractions were concentrated to dryness in vacuo to the title compound (3000 mg, 6.29 mmol, 79.71% yield) as a yellow oil. MS: 477.1 [M+H]+

### Intermediate 60

### tert-butyl 6-(4-amino-2-chloro-benzoyl)-2,6-diazaspiro[3.3]heptane-2-carboxylate

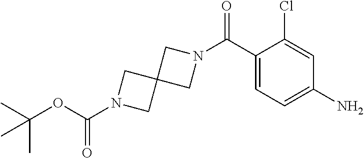 [SMILES: CC(C)(C)OC(=O)N1CC2(C1)CN(C(=O)c1ccc(N)cc1Cl)C2]

### Step 1: tert-butyl 6-(4-amino-2-chloro-benzoyl)-2,6-diazaspiro[3.3]heptane-2-carboxylate

To a stirred solution of 4-amino-2-chlorobenzoic acid (500 mg, 2.91 mmol, 1.0 eq), tert-butyl 2,6-diazaspiro[3.3]heptane-2-carboxylate (578 mg, 2.91 mmol, 1.0 eq) and N,N-diisopropylethylamine (1.02 mL, 5.83 mmol, 2.0 eq) in DMF (10 mL) was added O-(7-azabenzotriazol-1-yl)-N,N, N′,N′-tetramethyluronium hexafluorophosphate (1219 mg, 3.21 mmol, 1.1 eq) in one portion at 20° C. Then the solution was stirred at 20° C. for 1 h. The mixture was purified by reversed phase-HPLC (0.1% FA in water-ACN, B %=40%-60%) and lyophilized to afford the title compound (900 mg, 2.56 mmol, 87.78% yield). MS: 352.2, [M+H]+, ESI+

### Intermediate 61

### tert-butyl (2R,5R)-2-[4-(4-amino-2-chloro-benzoyl)piperazine-1-carbonyl]-5-methyl-pyrrolidine-1-carboxylate

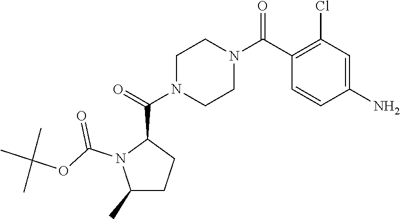 [SMILES: C[C@@H]1CC[C@H](C(=O)N2CCN(C(=O)c3ccc(N)cc3Cl)CC2)N1C(=O)OC(C)(C)C]

### Step 1: tert-butyl (2R,5R)-2-[4-(2-chloro-4-nitro-benzoyl)piperazine-1-carbonyl]-5-methyl-pyrrolidine-1-carboxylate

A solution of (2-chloro-4-nitro-phenyl)-piperazin-1-yl-methanone ([59939-73-0], 200 mg, 0.74 mmol, 1.0 eq) in DCM (5 mL) was added (2R,5R)-1-tert-butoxycarbonyl-5-methyl-pyrrolidine-2-carboxylic acid (170 mg, 0.74 mmol, 1.0 eq), 2-(7-azabenzotriazol-1-yl)-N,N,N′,N′-tetramethyluronium hexafluorophosphate (564 mg, 1.48 mmol, 2.0 eq) and N,N-diisopropylethylamine (287 mg, 2.22 mmol, 3.0 eq) the reaction was stirred at 25° C. for 5 h The reaction was concentrated to dryness and the residue was taken up in DCM (20 ml) and the organics washed with 2×10 ml water then 1×10 ml brine. The organics were then separated and dried (MgSO4) before concentration to dryness. The crude was then purified by silica gel chromatography eluting 70% EtOAc in Isohexane. The desired fractions were concentrated to dryness in vacuo to afford the title compound (265 mg, 0.55 mmol, 74.3% yield) as colorless oil. MS [M−Boc+H]+: 381.1

### Step 2: tert-butyl (2R,5R)-2-[4-(4-amino-2-chloro-benzoyl)piperazine-1-carbonyl]-5-methyl-pyrrolidine-1-carboxylate

To a solution of tert-butyl (2R,5R)-2-[4-(2-chloro-4-nitro-benzoyl)piperazine-1-carbonyl]-5-methyl-pyrrolidine-1-carboxylate (265 mg, 0.55 mmol, 1.0 eq) in ethanol (10 mL) and water (1 mL) was added iron powder (154 mg, 2.76 mmol, 5.0 eq) and ammonium chloride (147 mg, 2.76 mmol, 5.0 eq). The reaction was stirred at 50° C. for 16 h under nitrogen. The mixture was filtered through a Celite pad, and the filtrate was. The crude material was then purified by flash column chromatography eluting 10% MeOH in DCM. The desired fractions were concentrated to dryness in vacuo to afford the title compound (175 mg, 0.39 mmol, 70.43% yield) as colorless solid. MS [M+H]+: 451.3

### Intermediate 62

### tert-butyl N-[2-(6-amino-1-oxo-3,4-dihydroisoquinolin-2-yl)ethyl]carbamate

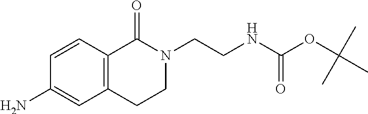 [SMILES: CC(C)(C)OC(=O)NCCN1CCc2cc(N)ccc2C1=O]

### Step 1: tert-butyl N-[2-(6-bromo-1-oxo-2-isoquinolyl)ethyl]carbamate

To a solution of 6-bromo-2H-isoquinolin-1-one (4 g, 17.85 mmol, 1.0 eq) in DMSO (100 mL) was added 60% sodium hydride (2.86 g, 71.41 mmol, 4.0 eq) at 25° C. After stirring for 30 min, tert-butyl N-(2-bromoethyl) carbamate (12 g, 53.56 mmol, 3.0 eq) was added, and the resulting mixture was stirred at 25° C. for 16 h. The resulting solution was diluted with 200 ml of EtOAc, then quenched by the addition of 300 mL of water. The organic layers were combined, washed with brine, dried and concentrated under vacuum. The residue silica gel chromatography eluting with ethyl acetate/PE (1/3) to afford the title comopund (1.3 g, 3.54 mmol, 19.83% yield). MS: 267.0, 269.0 [M−Boc+H]+

### Step 2: tert-butyl N-[2-[6-(benzhydrylideneamino)-1-oxo-2-isoquinolyl]ethyl]carbamate

A mixture of tert-butyl N-[2-(6-bromo-1-oxo-2-isoquinolyl)ethyl]carbamate (1500 mg, 4.08 mmol, 1.0 eq), tris(dibenzylideneacetone)dipalladium (815 mg, 0.82 mmol, 0.2 eq), cesium carbonate (3992 mg, 12.25 mmol, 3.0 eq) and diphenylmethanimine (1.03 mL, 6.13 mmol, 1.5 eq) in DMF (2 mL) was heated to 100° C. and stirred for 10 h under nitrogen atmosphere. The reaction was filtered, the filtrate was concentrated and purified by silica gel chromatography eluting 0˜60% EtOAc in PE to afford the title compound (1600 mg, 3.41 mmol, 83.42% yield). MS: 468.1 [M+H]+

### Step 3: tert-butyl N-[2-(6-amino-1-oxo-3,4-dihydroisoquinolin-2-yl)ethyl]carbamate

A solution of tert-butyl N-[2-[6-(benzhydrylideneamino)-1-oxo-2-isoquinolyl]ethyl]carbamate (1600 mg, 3.42 mmol, 1.0 eq) and Palladium on active carbon (535 mg) in ammonium hydroxide solution (6.0 mL) and methanol (40 mL) was stirred at 25° C. for 48 h. The resulting suspension was filtered and the filtrate was concentrated under vacuum. The residue was purified by silica gel chromatography eluting with EtOAc/PE (1/20) to afford the title compound (800 mg, 2.62 mmol, 76.56% yield) as a yellow oil. MS: 306.3 [M+H]+

### Intermediate 63

### tert-butyl 4-(4-piperidylsulfonimidoyl)piperidine-1-carboxylate

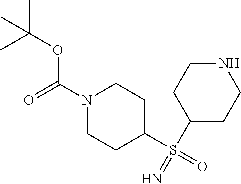 [SMILES: CC(C)(C)OC(=O)N1CCC(S(=N)(=O)C2CCNCC2)CC1]

### Step 1: benzyl 4-[(1-tert-butoxycarbonyl-4-piperidyl)sulfonimidoyl]piperidine-1-carboxylate

To a solution of benzyl 4-[(1-tert-butoxycarbonyl-4-piperidyl) sulfanyl]piperidine-1-carboxylate ([2099056-35-4], 400 mg, 0.92 mmol, 1.0 eq) in MeOH (5 mL) was added (NH₄)₂CO₃ (132 mg, 1.38 mmol, 1.5 eq) followed by PhI(OAc)₂ (681 mg, 2.12 mmol, 2.3 eq) under N₂. The reaction mixture was then stirred at 20° C. for 12 h. The reaction mixture was filtered, and the filtrate was concentrated under reduced pressure. The residue was purified by column silica gel column chromatography (PE:EtOAc 1:0 to 0:1) to afford the title compound (400 mg, 0.86 mmol, 75% yield) MS: m/z=466.2 [M+Na]+, ESI pos.

### Step 2: tert-butyl 4-(4-piperidylsulfonimidoyl)piperidine-1-carboxylate

Solution 1: {benzyl 4-[(1-tert-butoxycarbonyl-4-piperidyl)sulfonimidoyl]piperidine-1-carboxylate, 1 eq, 0.3 g} and {HOAc, 2 eq, 0.077 g} in {MeOH, 7.5 mL} and {THF, 7.5 mL}. The fixed bed (named FLR1, volume 5 mL) was completely packed with granular catalyst 5% Pd/Al₂O₃ (WXC1035, 3 g). The H₂ back pressure regulator was adjusted to 1.5 MPa, and the flow rate of H₂ was 30 mL/min. Then the solution S1 was pumped by Pump 1 {S1, P1, 0.3 mL/min} to fixed bed {FLR1, SS, Fixed bed, 6.350 (¼″) mm, 5 mL, 50° C.}. Then the reaction mixture was collected from the reactor output. The fixed bed was washed by extra {MeOH, 200 mL}. The combined filtrate was concentrated under reduced pressure to afford the title compound (400 mg, 1.21 mmol, 94% yield). 1H NMR (400 MHz, CD₃OD) δ 4.25 (d, J=7.2 Hz, 2H), 3.50-3.35 (m, 4H), 3.04-2.92 (m, 2H), 2.92-2.75 (m, 2H), 2.32-2.17 (m, 2H), 2.07 (d, J=10.8 Hz, 2H), 1.95 (d, J=1.6 Hz, 1H), 1.92-1.84 (m, 1H), 1.73-1.61 (m, 2H), 1.47 (s, 9H) ppm.

### Intermediate 64

### 2-[3-(9H-fluoren-9-ylmethoxycarbonyl)-3-azabicyclo[3.1.1]heptan-1-yl]acetic acid

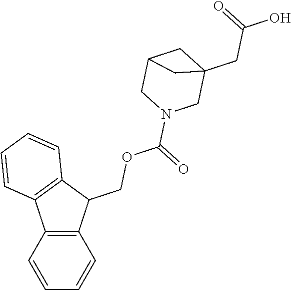 [SMILES: O=C(O)CC12CC(CN(C(=O)OCC3c4ccccc4-c4ccccc43)C1)C2]

### Step 1: 9H-fluoren-9-ylmethyl 1-chlorocarbonyl-3-azabicyclo[3.1.1]heptane-3-carboxylate

To a stirred solution of 3-(9H-fluoren-9-ylmethoxycarbonyl)-3-azabicyclo[3.1.1]heptane-1-carboxylic acid ([2375274-00-1], 100 mg, 0.28 mmol, 1.0 eq) in CH₂Cl₂ (1 mL) was added DMF (0.01 mL) at 0° C. Then oxalyl chloride (69 mg, 0.55 mmol, 2.0 eq) was dropwise added, the reaction mixture was stirred at 20° C. for 1 h. The reaction mixture was concentrated under reduced pressure to afford the title compound (100 mg, 0.26 mmol, 95% yield) as a yellow gum.

### Step 2: 2-[3-(9H-fluoren-9-ylmethoxycarbonyl)-3-azabicyclo[3.1.1]heptan-1-yl]acetic acid

To a solution of 9H-fluoren-9-ylmethyl 1-chlorocarbonyl-3-azabicyclo[3.1.1]heptane-3-carboxylate (300 mg, 0.79 mmol, 1.0 eq) in MeCN (25 mL) was added (trimethylsilyl)diazomethane (0.47 mL, 0.94 mmol, 1.2 eq), the mixture was stirred at 20° C. for 2 h. A solution of CF₃CO₂Ag (260 mg, 1.18 mmol, 1.5 eq) and triethylamine (0.22 mL, 1.57 mmol, 2.0 eq) in H₂O (3 mL) was added to reaction mixture, the resulting mixture was then stirred at 20° C. for 12 h under N₂. The reaction mixture was quenched with AcOH (20 mL), and the mixture was stirred at 20° C. for 10 min, then diluted with H₂O (100 mL), extracted with EtOAc (3×120 mL). The combined organic phases were washed with brine (200 mL), dried over Na₂SO₄, filtered and the filtrate was concentrated under reduced pressure. The residue was purified by silica gel column chromatography (PE:EtOAc 1:0 to 1:1) to give the title compound (70 mg, 0.19 mmol, 35% yield) as colorless oil. MS: m/z=378.1 [M+H]+, ESI pos.

### Intermediate 65

### piperazin-1-yl(1,4,5,6-tetrahydropyrimidin-5-yl)methanone; dihydrochloride

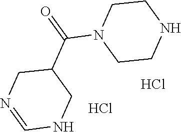 [SMILES: O=C(C1CN=CNC1)N1CCNCC1 ; Cl ; Cl]

### Step 1: tert-butyl 4-(1,4,5,6-tetrahydropyrimidine-5-carbonyl)piperazine-1-carboxylate

To a mixture of tert-butyl 4-(pyrimidine-5-carbonyl)piperazine-1-carboxylate (CAS: 2942774-49-2, 2000 mg, 6.84 mmol, 1.0 eq) in MeOH (50 mL) and HCl/MeOH (5.13 mL, 2 M) was added 5% Pd/SiO₂ under N₂. The reaction mixture was stirred at 25° C. for 16 h under H₂ (in balloon). The reaction mixture was concentrated under reduced pressure to afford the title compound (1.8 g, 6.07 mmol, 89% yield) as a yellow gum. MS: m/z=297.2 [M+H]+, ESI pos.

### Step 2: piperazin-1-yl(1,4,5,6-tetrahydropyrimidin-5-yl)methanone;dihydrochloride

A mixture of tert-butyl 4-(1,4,5,6-tetrahydropyrimidine-5-carbonyl)piperazine-1-carboxylate (200 mg, 0.67 mmol, 1.0 eq) in HCl/dioxane (1.0 mL, 2 M) was stirred at 20° C. for 2 h. The reaction mixture was concentrated under reduced pressure to afford the title compound (200 mg, 0.67 mmol, 1.0 eq). MS: m/z=197.1 [M+H]+, ESI pos.

### Intermediate 66

### Step 1: tert-butyl 4-(1-methyl-5,6-dihydro-4H-pyrimidine-5-carbonyl)piperazine-1-carboxylate

To a solution of tert-butyl 4-(1,4,5,6-tetrahydropyrimidine-5-carbonyl)piperazine-1-carboxylate (0.5 g, 1.69 mmol, 1.0 eq) in MeCN (10 mL) were added sodium hydrogen carbonate (708 mg, 8.44 mmol, 5.0 eq) and iodomethane (478.5 mg, 3.37 mmol, 2.0 eq). The reaction mixture was then stirred at 25° C. for 16 h. The reaction mixture was filtered and the filtrate was concentrated under reduced pressure. The residue was purified by reverse phase HPLC (H₂O (TFA)-MeCN; 10%-40%) to afford the title compound (300 mg, 0.97 mmol, 34% yield). MS: m/z=311.2 [M+H]+, ESI pos.

### Step 2: (1-methyl-5,6-dihydro-4H-pyrimidin-5-yl)-piperazin-1-yl-methanone;dihydrochloride

A solution of tert-butyl 4-(1-methyl-5,6-dihydro-4H-pyrimidine-5-carbonyl)piperazine-1-carboxylate (300 mg, 0.97 mmol, 1.0 eq) in HCl/dioxane (6.0 mL, 2 M) was stirred at 20° C. for 1 h. The reaction mixture was concentrated under reduced pressure to afford the title compound (270 mg, 0.95 mmol, 99% yield). MS: m/z=211.2 [M+H]+, ESI pos.

### Intermediate 67

### 5-amino-1-[2-(difluoromethyl)-3-fluoro-4-(fluoromethoxy)phenyl]-N-[3-fluoro-4-(piperazine-1-carbonyl)phenyl]imidazole-4-carboxamide;hydrochloride

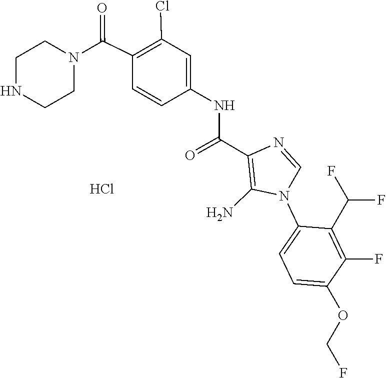 [SMILES: Nc1c(C(=O)Nc2ccc(C(=O)N3CCNCC3)c(Cl)c2)ncn1-c1ccc(OCF)c(F)c1C(F)F ; Cl]

### Step 1 tert-butyl 4-[4-[[5-(tert-butoxycarbonylamino)-1-[2-(difluoromethyl)-3-fluoro-4-(fluoromethoxy)phenyl]imidazole-4-carbonyl]amino]-2-fluoro-benzoyl]piperazine-1-carboxylate

A mixture of ethyl 5-[bis(tert-butoxycarbonyl)amino]-1-[2-(difluoromethyl)-3-fluoro-4-(fluoromethoxy)phenyl]imidazole-4-carboxylate (Intermediate 38, 3.05 g, 5.57 mmol, 1.0 eq) and tert-butyl 4-(4-amino-2-fluoro-benzoyl)piperazine-1-carboxylate ([1700051-29-1], 1.8 g, 5.57 mmol, 1.0 eq) in THF (30 mL) was evacuated and backfilled with N₂. Then the solution was cooled to −78° C. and lithium bis(trimethylsilyl)amide (22.27 mL, 22.27 mmol, 4.0 eq) was added dropwise. The resulting mixture was stirred at 20° C. for 16 h. The reaction mixture was poured into saturated aqueous 1M HCl (40 mL) and extracted with EtOAc (60 mL). Combined extracts were washed with brine (60 mL), dried over Na₂SO₄, filtered, and concentrated under reduced pressure. The residue was purified by column (PE:EtOAc=1:2 to 1:4, Rf=0.45, UV) to afford the title compound (1.5 g, 2.07 mmol, 37% yield). MS: m/z=725.3. [M+H]+, ESI pos.

### Step 2 5-amino-1-[2-(difluoromethyl)-3-fluoro-4-(fluoromethoxy)phenyl]-N-[3-fluoro-4-(piperazine-1-carbonyl)phenyl]imidazole-4-carboxamide;hydrochloride

A mixture of tert-butyl 4-[4-[[5-(tert-butoxycarbonylamino)-1-[2-(difluoromethyl)-3-fluoro-4-(fluoromethoxy)phenyl]imidazole-4-carbonyl]amino]-2-fluoro-benzoyl]piperazine-1-carboxylate (1.5 g, 2.07 mmol, 1.0 eq) in HCl/dioxane (30 mL, 2M) was stirred at 20° C. for 4 h. The mixture was concentrated under reduced pressure. The residue was purified by reverse phase HPLC (H₂O (0.1% HCl)-MeCN B %: 40%-60%) to afford the title compound (1 g, 1.78 mmol, 86% yield). MS: m/z=525.1. [M+H]+, ESI pos.

The following compounds were prepared in analogy to the procedure above

| | | | | MS |
|---|---|---|---|---|
| Int | Structure | Name | Reagents | [M + H]+ |
| 68 | 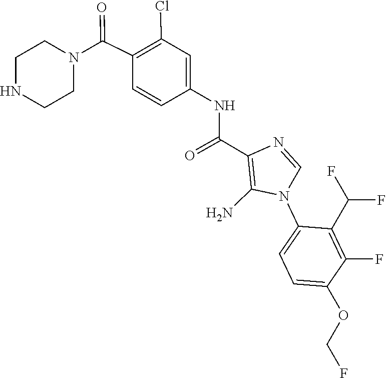 [SMILES: Nc1c(C(=O)Nc2ccc(C(=O)N3CCNCC3)c(Cl)c2)ncn1-c1ccc(OCF)c(F)c1C(F)F] | 5-amino-N- [3-chloro-4- (piperazine-1- carbonyl) phenyl]-1-[2- (difluoromethyl)-3- fluoro-4- (fluoromethoxy) phenyl] imidazole-4- carboxamide; hydrochloride | Intermediate 38, tert-butyl 4-(4- amino-2-chloro- benzoyl)piperazine- 1-carboxylate [1338249-09-4] | 541.1 |
| 69 | 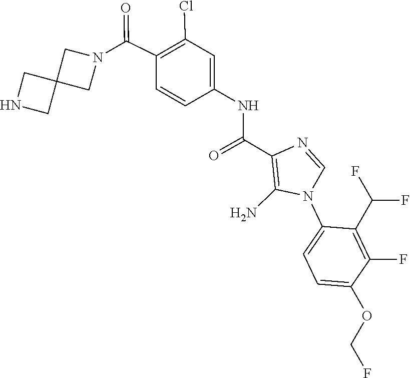 [SMILES: Nc1c(C(=O)Nc2ccc(C(=O)N3CC4(CNC4)C3)c(Cl)c2)ncn1-c1ccc(OCF)c(F)c1C(F)F] | 5-amino-N- [3-chloro-4- (2,6- diazaspiro[3.3] heptane- 2-carbonyl) phenyl]-1-[2- (difluoromethyl)-3- fluoro-4- (fluoromethoxy) phenyl] imidazole-4- carboxamide | Intermediate 38, Intermediate 60 | 553.2 |
| 70 | 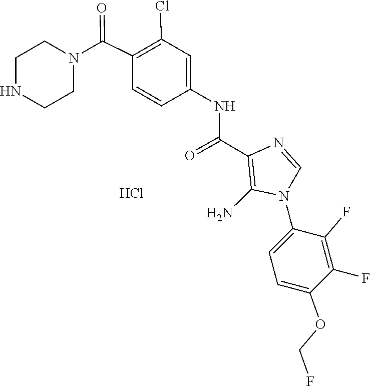 [SMILES: Nc1c(C(=O)Nc2ccc(C(=O)N3CCNCC3)c(Cl)c2)ncn1-c1ccc(OCF)c(F)c1F ; Cl] | 5-amino-N- [3-chloro-4- (piperazine-1- carbonyl)phenyl]- 1-[2,3- difluoro-4- (fluoromethoxy) phenyl] imidazole-4- carboxamide; hydrochloride | Intermediate 42, tert-butyl 4-(4- amino-2-chloro- benzoyl)piperazine- 1-carboxylate [1338249-09-4] | 509.2 |
| 71 | 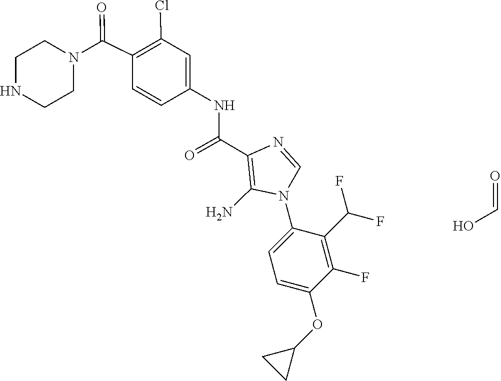 [SMILES: Nc1c(C(=O)Nc2ccc(C(=O)N3CCNCC3)c(Cl)c2)ncn1-c1ccc(OC2CC2)c(F)c1C(F)F ; O=CO] | 5-amino-N- [3-chloro-4- (piperazine-1- carbonyl) phenyl]-1-[4- (cyclopropoxy)-2- (difluoromethyl)-3- fluorophenyl] imidazole- 4-carboxamide; formic acid | Intermediate 39, tert-butyl 4-(4- amino-2-chloro- benzoyl)piperazine- 1-carboxylate [1338249-09-4] | 549.1 |
| 72 | 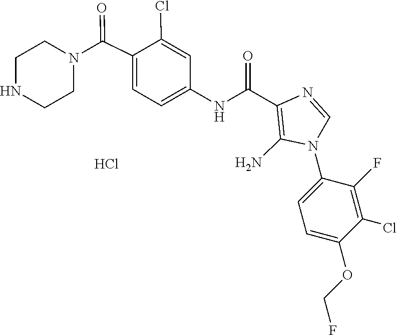 [SMILES: Nc1c(C(=O)Nc2ccc(C(=O)N3CCNCC3)c(Cl)c2)ncn1-c1ccc(OCF)c(Cl)c1F ; Cl] | 5-amino-1- [3-chloro-2- fluoro-4- (fluoromethoxy) phenyl]- N-[3-chloro-4- (piperazine-1- carbonyl)phenyl] imidazole-4- carboxamide; hydrochloride | Intermediate 44, tert-butyl 4-(4- amino-2-chloro- benzoyl)piperazine- 1-carboxylate [1338249-09-4] | 525.1 |
| 73 | 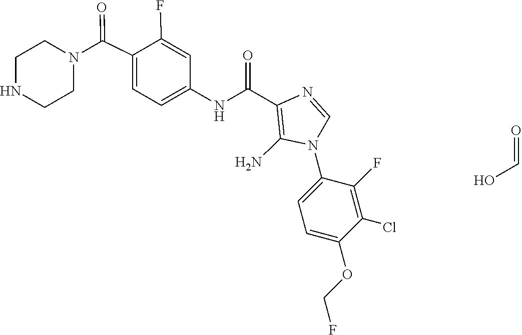 [SMILES: Nc1c(C(=O)Nc2ccc(C(=O)N3CCNCC3)c(F)c2)ncn1-c1ccc(OCF)c(Cl)c1F ; O=CO] | 5-amino-1- [3-chloro-2- fluoro-4- (fluoromethoxy) phenyl]- N-[3-fluoro-4- (piperazine-1- carbonyl)phenyl] imidazole-4- carboxamide; formic acid | Intermediate 44, tert-butyl 4-(4- amino-2-fluoro- benzoyl)piperazine- 1-carboxylate [1700051-29-1] | 509.3 |

### Intermediate 74

### 4-[[5-amino-1-[2,3-difluoro-4-(fluoromethoxy)phenyl]imidazole-4-carbonyl]amino]-2-chloro-benzoic acid;hydrochloride

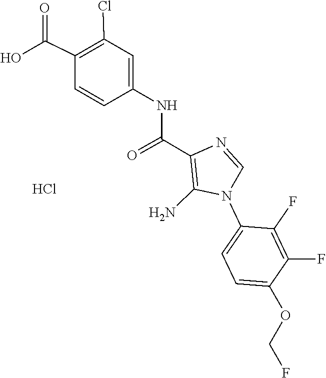 [SMILES: Nc1c(C(=O)Nc2ccc(C(=O)O)c(Cl)c2)ncn1-c1ccc(OCF)c(F)c1F ; Cl]

### Step 1 tert-butyl 4-[[5-(tert-butoxycarbonylamino)-1-[2,3-difluoro-4-(fluoromethoxy)phenyl]imidazole-4-carbonyl]amino]-2-chlorobenzoate

To a solution of ethyl 5-[bis(tert-butoxycarbonyl)amino]-1-[2,3-difluoro-4-(fluoromethoxy)phenyl]imidazole-4-carboxylate (Intermediate 42, 1 g, 1.94 mmol, 1.0 eq) and tert-butyl 4-amino-2-chlorobenzoate ([75294-49-4], 662 mg, 2.91 mmol, 1.5 eq) in THF (10 mL) was added dropwise LiHMDS (5.82 mL, 5.82 mmol, 3.0 eq) at −60° C. under N₂. The mixture was then stirred at −60° C. for 2 h under N₂. The reaction mixture was quenched with saturated NH₄Cl solution (40 mL) at 0° C. EtOAc (30 mL) and H₂O (20 mL) were added and layers were separated. The aqueous phase was extracted with EtOAc (2×30 mL). Combined extracts were washed with brine (3×30 mL), dried over Na₂SO₄, filtered, and concentrated under reduced pressure. The residue was purified by column silica gel column chromatography (PE:EtOAc=0:1 to 5:1) to afford the title compound (1.1 g, 1.84 mmol, 91% yield). MS: m/z=597.2. [M+H]+, ESI pos.

### Step 2 4-[[5-amino-1-[2,3-difluoro-4-(fluoromethoxy)phenyl]imidazole-4-carbonyl]amino]-2-chloro-benzoic acid;hydrochloride

To a solution of tert-butyl 4-[[5-(tert-butoxycarbonylamino)-1-[2,3-difluoro-4-(fluoromethoxy)phenyl]imidazole-4-carbonyl]amino]-2-chlorobenzoate (1.1 g, 1.32 mmol, 1.0 eq) in 1,4-dioxane (5 mL) was added HCl/dioxane (31.61 mL, 4 M) at 25° C. The mixture was then stirred at 25° C. for 4 h. The mixture was concentrated under reduced pressure to afford the title compound (600 mg, 1.26 mmol, 94% yield). MS: m/z=441.0. [M+H]+, ESI pos.

The following compound was prepared in analogy to the procedure above

| Int | Structure | Name | Reagents | MS [M + H]+ |
|---|---|---|---|---|
| 75 | 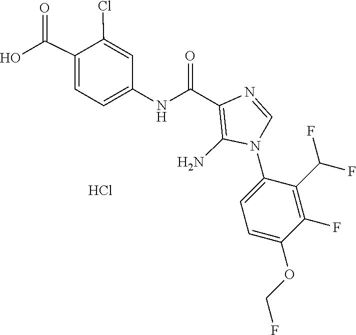 [SMILES: Nc1c(C(=O)Nc2ccc(C(=O)O)c(Cl)c2)ncn1-c1ccc(OCF)c(F)c1C(F)F ; Cl] | 4-[[5-amino-1-[2- (difluoromethyl)-3- fluoro-4- (fluoromethoxy) phenyl]imidazole-4- carbonyl]amino]- 2-chloro-benzoic acid;hydrochloride | Intermediate 38, tert-butyl 4-(4- amino-2-chloro- benzoyl)piperazine- 1-carboxylate [1338249-09-4] | 473.0 |

### Intermediate 76

### 1-[4-[[5-amino-1-[2,3-difluoro-4-(fluoromethoxy)phenyl]imidazole-4-carbonyl]amino]-2-chlorobenzoyl]piperidine-4-carboxylic acid

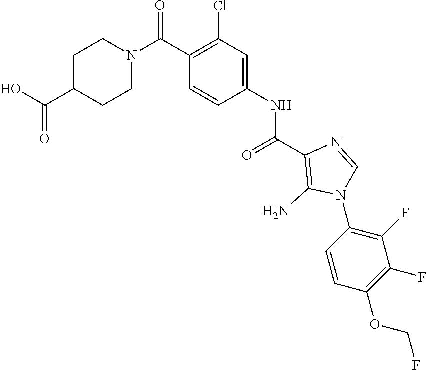 [SMILES: Nc1c(C(=O)Nc2ccc(C(=O)N3CCC(C(=O)O)CC3)c(Cl)c2)ncn1-c1ccc(OCF)c(F)c1F]

### Step 1: methyl 1-[4-[[5-amino-1-[2,3-difluoro-4-(fluoromethoxy)phenyl]imidazole-4-carbonyl]amino]-2-chloro-benzoyl]piperidine-4-carboxylate

To a solution of methyl 1-(4-amino-2-chloro-benzoyl)piperidine-4-carboxylate ([1307277-54-8], 97 mg, 0.33 mmol, 1.0 eq) and pyridine (0.53 mL, 6.54 mmol, 20.0 eq) in DCM (1 mL) was added a suspension of 5-amino-1-[2,3-difluoro-4-(fluoromethoxy)phenyl]imidazole-4-carbonyl chloride (100 mg, 0.33 mmol, 1.0 eq) in DCM (1 mL) at 25° C. The reaction was concentrated to dryness and the residue was taken up in DCM (3 ml) and the organics washed with 2×3 ml water then 1×3 ml saturated brine solution. The organics were then separated and dried (Na2SO4) before concentration to dryness. The crude was then purified by flash column chromatography eluting 4% MeOH in DCM. The desired fractions were concentrated to dryness in vacuo to afford the title compound (140 mg, 0.25 mmol, 74.85% yield). MS: 565.9 [M+H]+

### Step 2: 1-[4-[[5-amino-1-[2,3-difluoro-4-(fluoromethoxy)phenyl]imidazole-4-carbonyl]amino]-2-chlorobenzoyl]piperidine-4-carboxylic acid

To a solution of sodium hydroxide (67 mg, 1.68 mmol, 5.0 eq) was added to methyl 1-[4-[[5-amino-1-[2,3-difluoro-4-(fluoromethoxy)phenyl]imidazole-4-carbonyl]amino]-2-chloro-benzoyl]piperidine-4-carboxylate (190 mg, 0.34 mmol, 1.0 eq) in IPA (2 mL) and water (1 mL), the resultant mixture was stirred at 50° C. for 2 h. The mixture was adjusted to pH 5-6 with 2N aqueous HCl and extracted with EtOAc. The reaction mixture was partitioned between EtOAc and water. The aqueous layer was extracted again with EA. The combined organic layer was dried over anhydrous sodium sulfate and concentrated under vacuum to afford the title compound (150 mg, 0.27 mmol, 79.97% yield). MS: 552.2 [M+H]+

### Intermediate 77

### 5-amino-N-[3-chloro-4-(piperazine-1-carbonyl)phenyl]-1-[3-(trifluoromethyl)-1H-pyrazol-4-yl]imidazole-4-carboxamide;hydrochloride

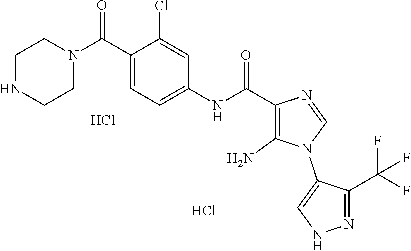 [SMILES: Nc1c(C(=O)Nc2ccc(C(=O)N3CCNCC3)c(Cl)c2)ncn1-c1c[nH]nc1C(F)(F)F ; Cl ; Cl]

### Step 1 tert-butyl 4-[[5-(tert-butoxycarbonylamino)-1-[2,3-difluoro-4-(fluoromethoxy)phenyl]imidazole-4-carbonyl]amino]-2-chlorobenzoate

To a solution of tert-butyl 4-(4-amino-2-chloro-benzoyl)piperazine-1-carboxylate ([1338249-09-4], 2.09 g, 6.15 mmol, 1.5 eq) and ethyl 5-[bis(tert-butoxycarbonyl)amino]-1-[3-(trifluoromethyl)-1-trityl-pyrazol-4-yl]imidazole-4-carboxylate (Intermediate 45, 3 g, 4.1 mmol, 1.0 eq) in THF (20 mL) was added dropwise LiHMDS (12.3 mL, 12.3 mmol, 3.0 eq) at −70° C. under N₂. The mixture was then stirred at 20° C. for 3.5 h under N₂. The reaction mixture was quenched with saturated NH₄Cl solution (40 mL) by stirring at 0° C. EtOAc (50 mL) and H₂O (50 mL) were added, and the layers were separated. The aqueous phase was extracted with EtOAc (2×50 mL). Combined extracts were washed with brine (3×50 mL), dried over Na₂SO₄, filtered, and concentrated under reduced pressure. The residue was purified by reverse phase HPLC (0.1% FA in H₂O-MeCN, 100% MeCN) to afford the title compound (3.5 g, 3.78 mmol, 92% yield). MS: m/z=925.4 [M+H]+, ESI pos.

### Step 2 5-amino-N-[3-chloro-4-(piperazine-1-carbonyl)phenyl]-1-[3-(trifluoromethyl)-1H-pyrazol-4-yl]imidazole-4-carboxamide;hydrochloride

A solution of tert-butyl 4-[4-[[5-(tert-butoxycarbonylamino)-1-[3-(trifluoromethyl)-1-trityl-pyrazol-4-yl]imidazole-4-carbonyl]amino]-2-chloro-benzoyl]piperazine-1-carboxylate (3.5 g, 3.78 mmol, 1.0 eq) in HCl/dioxane (68.76 mL, 4M) was stirred at 25° C. for 16 h. Then was concentrated under reduced pressure, The residue was triturated in MTBE (30 mL) and filtered to afford the title compound (1.7 g, 3.27 mmol, 87% yield). MS: m/z=483.2 [M+H]+, ESI pos.

The following compounds were prepared in analogy to the procedure above

| | | | | MS |
|---|---|---|---|---|
| Int | Structure | Name | Reagents | [M + H]+ |
| 78 | 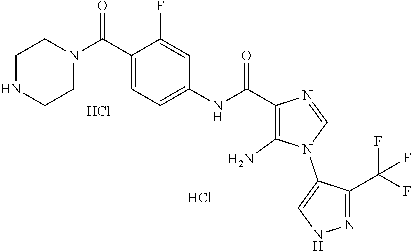 [SMILES: Nc1c(C(=O)Nc2ccc(C(=O)N3CCNCC3)c(F)c2)ncn1-c1c[nH]nc1C(F)(F)F ; Cl ; Cl] | 5-amino-N-[3-fluoro-4- (piperazine-1- carbonyl)phenyl]-1-[3- (trifluoromethyl)-1H- pyrazol-4-yl]imidazole- 4- carboxamide; dihydrochloride | Intermediate 45, tert-butyl 4-(4- amino-2-fluoro- benzoyl)piperazine- 1-carboxylate [1700051-29-1] | 467.2 |
| 79 | 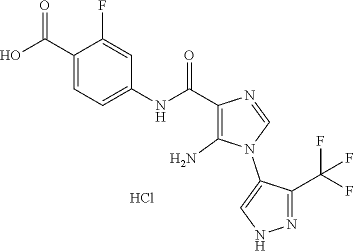 [SMILES: Nc1c(C(=O)Nc2ccc(C(=O)O)c(F)c2)ncn1-c1c[nH]nc1C(F)(F)F ; Cl] | 4-[[5-amino-1-[3- (trifluoromethyl)-1H- pyrazol-4-yl]imidazole- 4-carbonyl]amino]-2- fluoro-benzoic acid;hydrochloride | Intermediate 45, tert-butyl 4-amino- 2-fluoro-benzoate [140373-77-9] | 399.1 |

### Intermediate 80

### tert-butyl 4-[4-[[5-amino-1-[3-(trifluoromethyl)-1H-pyrazol-4-yl]imidazole-4-carbonyl]amino]-2-chloro-benzoyl]piperazine-1-carboxylate

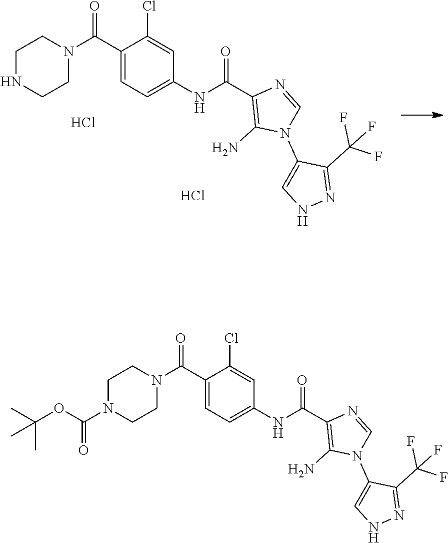 [SMILES: Nc1c(C(=O)Nc2ccc(C(=O)N3CCNCC3)c(Cl)c2)ncn1-c1c[nH]nc1C(F)(F)F ; Cl ; Cl ; CC(C)(C)OC(=O)N1CCN(C(=O)c2ccc(NC(=O)c3ncn(-c4c[nH]nc4C(F)(F)F)c3N)cc2Cl)CC1]

To a solution of 5-amino-N-[3-chloro-4-(piperazine-1-carbonyl)phenyl]-1-[3-(trifluoromethyl)-1H-pyrazol-4-yl]imidazole-4-carboxamide;hydrochloride (Intermediate 77, 2.5 g, 4.81 mmol, 1.0 eq) in CH₂Cl₂ (20 mL) were added (Boc) 20 (1155.74 mg, 5.3 mmol, 1.1 eq) and triethylamine (2.01 mL, 14.44 mmol, 3.0 eq). The resulting solution was stirred at 25° C. for 2 h. The solution was concentrated under reduced pressure, the residue was purified by reverse phase HPLC (H₂O (0.1% FA)-MeCN B %: 50-65%) to afford the title compound (1.8 g, 3.09 mmol, 64% yield). MS: m/z=583.3 [M+H]+, ESI pos.

### Intermediate 81

### tert-butyl 4-[4-[4-[[5-amino-1-[3-(trifluoromethyl)-1H-pyrazol-4-yl]imidazole-4-carbonyl]amino]-2-chlorobenzoyl]piperazine-1-carbonyl]piperidine-1-carboxylate

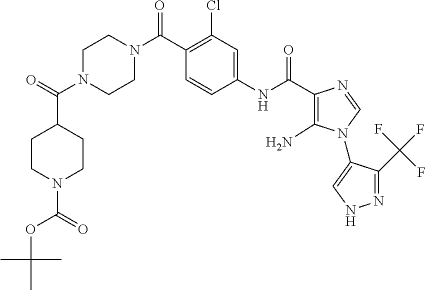 [SMILES: CC(C)(C)OC(=O)N1CCC(C(=O)N2CCN(C(=O)c3ccc(NC(=O)c4ncn(-c5c[nH]nc5C(F)(F)F)c4N)cc3Cl)CC2)CC1]

To a solution of 5-amino-N-[3-chloro-4-(piperazine-1-carbonyl)phenyl]-1-[3-(trifluoromethyl)-1H-pyrazol-4-yl]imidazole-4-carboxamide (200 mg, 0.41 mmol, 1.0 eq) in DCM (5 mL) were added N-BOC-isonipecotic acid (114 mg, 0.5 mmol, 1.2 eq), 50% propyl phosphate anhydride (395 mg, 0.62 mmol, 1.5 eq) and N,N-diisopropylethylamine (0.29 mL, 1.66 mmol, 4.0 eq), the resultant mixture was stirred at 0° C. for 1 h, the solvent was removed under vacuo, the residue was diluted with EtOAc (10 mL) and washed with H₂O (10 mL×4), dried over Na₂SO₄ and concentrated under vacuo to afford the title compound (210 mg, 0.3 mmol, 73.04% yield). MS: 594.0 [M−Boc+H]+

### Intermediate 82

### 1-[4-[[5-amino-1-[3-(trifluoromethyl)-1H-pyrazol-4-yl]imidazole-4-carbonyl]amino]-2-chloro-benzoyl]piperidine-4-carboxylic acid

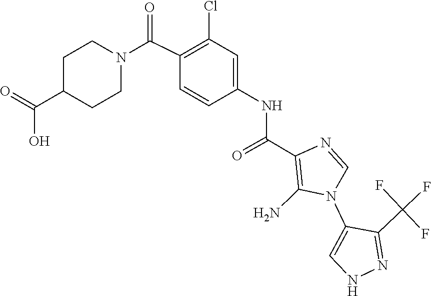 [SMILES: Nc1c(C(=O)Nc2ccc(C(=O)N3CCC(C(=O)O)CC3)c(Cl)c2)ncn1-c1c[nH]nc1C(F)(F)F]

### Step 1: methyl 1-[4-[[5-amino-1-[1-[(4-methoxyphenyl)methyl]-3-(trifluoromethyl)pyrazol-4-yl]imidazole-4-carbonyl]amino]-2-chloro-benzoyl]piperidine-4-carboxylate

To a solution of methyl 1-(4-amino-2-chloro-benzoyl)piperidine-4-carboxylate ([1307277-54-8], 624 mg, 2.1 mmol, 0.7 eq) and pyridine (4.86 mL, 60.04 mmol, 20.0 eq) in DCM (1 mL) was added a suspension of 5-amino-1-[1-[(4-methoxyphenyl)methyl]-3-(trifluoromethyl)pyrazol-4-yl]imidazole-4-carbonyl chloride (1200 mg, 3.0 mmol, 1.0 eq) in DCM (1 mL) at 25° C. The mixture was stirred at 25° C. for 1 h under N₂. The resulting solution was evaporated under reduced pressure. The residue was purified by column chromatography (silica gel, 0 to 10% MeOH in DCM) to afford the title compound (1123 mg, 1.7 mmol, 56.68% yield). MS: m/z=659.9 [M+H]+

### Step 2: methyl 1-[4-[[5-amino-1-[3-(trifluoromethyl)-1H-pyrazol-4-yl]imidazole-4-carbonyl]amino]-2-chloro-benzoyl]piperidine-4-carboxylate

A solution of methyl 1-[4-[[5-amino-1-[1-[(4-methoxyphenyl)methyl]-3-(trifluoromethyl)pyrazol-4-yl]imidazole-4-carbonyl]amino]-2-chloro-benzoyl]piperidine-4-carboxylate (1.12 g, 1.7 mmol, 1.0 eq) in trifluoroacetic acid (5.32 mL, 69.01 mmol, 40.56 eq) the reaction was stirred at 70° C. for 8 h. The resulting solution was evaporated under reduced pressure. The residue was purified by silica gel chromatography (0 to 10% MeOH in DCM) to afford the title compound (650 mg, 1.2 mmol, 70.76% yield). MS: 540.0 [M+H]+

### Step 3: 1-[4-[[5-amino-1-[3-(trifluoromethyl)-1H-pyrazol-4-yl]imidazole-4-carbonyl]amino]-2-chloro-benzoyl]piperidine-4-carboxylic acid

To a solution of methyl 1-[4-[[5-amino-1-[3-(trifluoromethyl)-1H-pyrazol-4-yl]imidazole-4-carbonyl]amino]-2-chloro-benzoyl]piperidine-4-carboxylate (650 mg, 1.2 mmol, 1.0 eq) and sodium hydroxide (240 mg, 6.02 mmol, 5.0 eq) in IPA (5 mL) were added sodium hydroxide (240 mg, 6.02 mmol, 5.0 eq) and water (1.5 mL). The resultant mixture was stirred at 50° C. for 4 h. The resulting solution was diluted with water and TBME. The layers were seaparated. The pH of the aq layer acidified and the resulting suspension filtered and dried to afford the title compound (510 mg, 0.97 mmol, 80.55% yield). MS: 526.0 [M+H]+

### Intermediate 83

### 5-amino-N-[3-chloro-4-(piperazine-1-carbonyl)phenyl]-1-[1-(cyclopropylmethyl)-3-(trifluoromethyl)pyrazol-4-yl]imidazole-4-carboxamide;hydrochloride

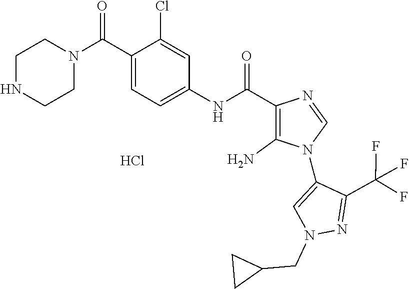 [SMILES: Nc1c(C(=O)Nc2ccc(C(=O)N3CCNCC3)c(Cl)c2)ncn1-c1cn(CC2CC2)nc1C(F)(F)F ; Cl]

### Step 1: tert-butyl 4-[4-[[5-amino-1-[1-(cyclopropylmethyl)-3-(trifluoromethyl)pyrazol-4-yl]imidazole-4-carbonyl]amino]-2-chloro-benzoyl]piperazine-1-carboxylate

To a solution of tert-butyl 4-[4-[[5-amino-1-[3-(trifluoromethyl)-1H-pyrazol-4-yl]imidazole-4-carbonyl]amino]-2-chloro-benzoyl]piperazine-1-carboxylate (Intermediate 80, 420 mg, 0.72 mmol, 1.0 eq) and K₂CO₃ (199 mg, 1.44 mmol, 2.0 eq) in MeCN (5 mL) was added ((bromomethyl)cyclopropane (0.07 mL, 0.72 mmol, 1.0 eq). The mixture was then stirred at 50° C. for 16 h. The mixture was poured into H₂O (90 mL) and extracted with EtOAc (3×30 mL). The combined organic phase was washed with brine (3×30 mL), dried over Na₂SO₄, filtered and concentrated under reduced pressure. The residue was purified by prep-HPLC (Welch ultimate XB-NH2 250×50×10 um, Hexane-EtOH, B %=10%-50%) to afford the title compound (340 mg, 0.53 mmol, 74% yield). MS: m/z=637.2 [M+H]+, ESI pos.

### Step 2: 5-amino-N-[3-chloro-4-(piperazine-1-carbonyl)phenyl]-1-[1-(cyclopropylmethyl)-3-(trifluoromethyl)pyrazol-4-yl]imidazole-4-carboxamide;hydrochloride

A solution of tert-butyl 4-[4-[[5-amino-1-[1-(cyclopropylmethyl)-3-(trifluoromethyl)pyrazol-4-yl]imidazole-4-carbonyl]amino]-2-chloro-benzoyl]piperazine-1-carboxylate (300 mg, 0.47 mmol, 1.0 eq) in HCl/dioxane (5.0 mL, 2 M) was stirred at 25° C. for 2 h. The mixture was concentrated under reduced pressure to afford the title compound (250 mg, 0.44 mmol, 93% yield). MS: m/z=537.1 [M+H]+, ESI pos.

The following compounds were prepared in analogy to the procedure above

| | | | | MS |
|---|---|---|---|---|
| Int | Structure | Name | Reagents | [M + H]+ |
| 84 | 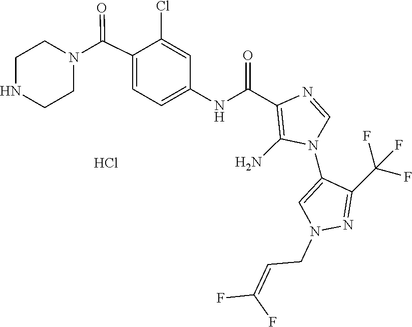 [SMILES: Nc1c(C(=O)Nc2ccc(C(=O)N3CCNCC3)c(Cl)c2)ncn1-c1cn(CC=C(F)F)nc1C(F)(F)F ; Cl] | 5-amino-N-[3-chloro-4- (piperazine-1- carbonyl)phenyl]-1-[1- (3,3-difluoroallyl)-3- (trifluoromethyl)pyrazol- 4-yl]imidazole-4- carboxamide;hydrochloride | Intermediate 80 3-bromo-3,3- difluoro-prop-1- ene [420-90-6] | 559.1 |
| 85 | 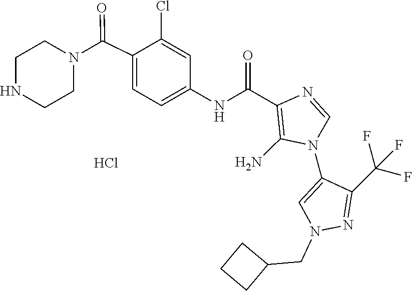 [SMILES: Nc1c(C(=O)Nc2ccc(C(=O)N3CCNCC3)c(Cl)c2)ncn1-c1cn(CC2CCC2)nc1C(F)(F)F ; Cl] | 5-amino-N-[3-chloro-4- (piperazine-1- carbonyl)phenyl]-1-[1- (cyclobutylmethyl)-3- (trifluoromethyl)pyrazol- 4-yl]imidazole-4- carboxamide;hydrochloride | Intermediate 80 (bromomethyl) cyclobutane [17247-58-4] | 551.3 |

### Intermediate 86

### tert-butyl 4-[2-[6-[[5-amino-1-[3-(trifluoromethyl)-1H-pyrazol-4-yl]imidazole-4-carbonyl]amino]-1-oxo-3,4-dihydroisoquinolin-2-yl]ethylcarbamoyl]-4-fluoro-piperidine-1-carboxylate

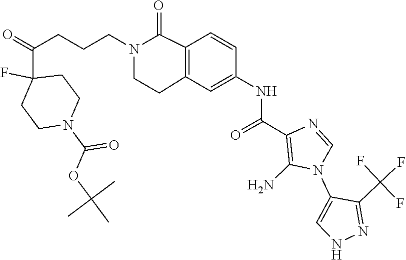 [SMILES: CC(C)(C)OC(=O)N1CCC(F)(C(=O)CCCN2CCc3cc(NC(=O)c4ncn(-c5c[nH]nc5C(F)(F)F)c4N)ccc3C2=O)CC1]

### Step 1: tert-butyl N-[2-[6-[[5-amino-1-[1-[(4-methoxyphenyl)methyl]-3-(trifluoromethyl)pyrazol-4-yl]imidazole-4-carbonyl]amino]-1-oxo-3,4-dihydroisoquinolin-2-yl]ethyl]carbamate

To a solution of tert-butyl N-[2-(6-amino-1-oxo-3,4-dihydroisoquinolin-2-yl)ethyl]carbamate (Intermediate 62, 305 mg, 1.0 mmol, 0.8 eq) and pyridine (1978 mg, 25.02 mmol, 20.0 eq) in DCM (5 mL) was was added a solution of 5-amino-1-[1-[(4-methoxyphenyl)methyl]-3-(trifluoromethyl)pyrazol-4-yl]imidazole-4-carbonyl chloride (500 mg, 1.25 mmol, 1.0 eq) in DCM (5 mL) at 25° C. and the mixture was stirred for 1 h under N₂. The resulting solution was concentrated under vacuum. The residue was purified by silica gel chromatography eluting with DCM/MeOH (10:1) to afford the title compound (420 mg, 0.63 mmol, 45.2% yield). MS 669.2 [M+H]+

### Step 2: 5-amino-N-[2-(2-aminoethyl)-1-oxo-3,4-dihydroisoquinolin-6-yl]-1-[3-(trifluoromethyl)-1H-pyrazol-4-yl]imidazole-4-carboxamide

A solution of tert-butyl N-[2-[6-[[5-amino-1-[1-[(4-methoxyphenyl)methyl]-3-(trifluoromethyl)pyrazol-4-yl]imidazole-4-carbonyl]amino]-1-oxo-3,4-dihydroisoquinolin-2-yl]ethyl]carbamate (420 mg, 0.63 mmol, 1.0 eq) in trifluoroacetic acid (5.0 mL, 64.9 mmol, 103.33 eq) the was stirred at 70° C. for 8 h. The reaction was concentrated to dryness to afford the title compound (220 mg, 0.49 mmol, 78.11% yield). MS: 449.0 [M+H]+

### Step 3: tert-butyl 4-[2-[6-[[5-amino-1-[3-(trifluoromethyl)-1H-pyrazol-4-yl]imidazole-4-carbonyl]amino]-1-oxo-3,4-dihydroisoquinolin-2-yl]ethylcarbamoyl]-4-fluoro-piperidine-1-carboxylate

To a solution of 5-amino-N-[2-(2-aminoethyl)-1-oxo-3,4-dihydroisoquinolin-6-yl]-1-[3-(trifluoromethyl)-1H-pyrazol-4-yl]imidazole-4-carboxamide (210 mg, 0.47 mmol, 1.0 eq) in DCM (1 mL) were added 1-BOC-4-fluoro-4-piperidinecarboxylic acid (116 mg, 0.47 mmol, 1 eq), N-butyl phosphate anhydride (674 mg, 0.94 mmol, 2.0 eq) and N,N-diisopropylethylamine (0.24 mL, 1.4 mmol, 3.0 eq), the resultant mixture was stirred at 25° C. for 1 h, the solvent was removed under vacuo, the residue was diluted with EtOAc (10 mL) and washed with H₂O (10 mL×4), dried over Na₂SO₄ and concentrated under vacuo to afford the title compound (260 mg, 0.38 mmol, 81.93% yield). MS 578.1 [M−Boc+H]+

### Intermediate 87

### 2-[3-(hydroxymethyl) azetidin-1-yl]-1,4,5,6-tetrahydropyrimidine-5-carboxylic acid;2,2,2-trifluoroacetic acid

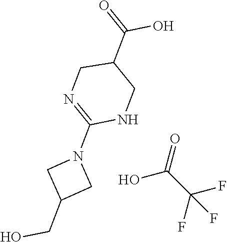

### Step 1: 2-[3-(hydroxymethyl) azetidin-1-yl]pyrimidine-5-carboxylic acid

To a mixture of 2-chloropyrimidine-5-carboxylic acid (0.5 g, 3.15 mmol, 1.0 eq) and azetidin-3-ylmethanol hydrochloride (506 mg, 4.1 mmol, 1.3 eq) in MeCN (10 mL) was added K₂CO₃ (1.31 g, 9.46 mmol, 3.0 eq) under N₂. The reaction mixture was then stirred for 16 h at 80° C. The reaction mixture was cooled to RT and concentrated under reduced pressure. The residue was resolved into H₂O (5 ml) and acidified to pH 3-4 by 1 M HCl. During this period, a white precipitate was formed. It was collected by filtration and dried under reduced pressure to afford the title compound (400 mg, 1.91 mmol, 58% yield). MS: m/z=209.9 [M+H]+, ESI pos.

### Step 2: 2-[3-(hydroxymethyl) azetidin-1-yl]-1,4,5,6-tetrahydropyrimidine-5-carboxylic acid;2,2,2-trifluoroacetic acid

Solution 1:2-[3-(hydroxymethyl) azetidin-1-yl]pyrimidine-5-carboxylic acid (200.0 mg, 0.96 mmol, 1.0 eq) and TFA (0.14 mL, 1.91 mmol, 2.0 eq) in MeOH (35 mL) The fixed bed (named FLR1, volume 5 mL) was completely packed with granular catalyst 5% Pd/SiO₂ (1.00 eq) 3 g. The H₂ back pressure regulator was adjusted to 0.5 MPa, and the flow rate of H₂ was 20 mL/min. Then the solution S1 was pumped by Pump 1 {S1, P1, 0.4 mL/min} to fixed bed {FLR1, SS, Fixed bed, 6.350 (¼″) mm, 1 mL, 35° C.}. Then the reaction mixture was collected from the reactor output. Stop collecting the reaction mixture after 180 min. Then the reaction mixture was collected from the reactor output and concentrated under reduced pressure to afford the title compound (250 mg, 1.17 mmol, 86% yield) as yellow oil. 1H NMR (400 MHz, CD₃OD) δ 4.10 (t, J=8.4 Hz, 2H), 3.85 (dd, J=5.6, 8.4 Hz, 2H), 3.69 (d, J=5.6 Hz, 2H), 3.60-3.51 (m, 4H), 3.08-3.00 (m, 1H), 2.90 (tt, J=5.6, 8.4 Hz, 1H) ppm.

The following compounds were prepared in analogy to the procedure above

| | | | | MS |
|---|---|---|---|---|
| Int | Structure | Name | Reagents | [M + H]+ |
| 88 | 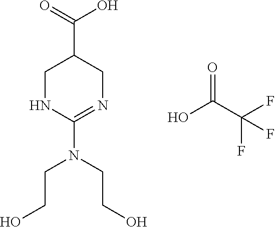 [SMILES: O=C(O)C1CN=C(N(CCO)CCO)NC1 ; O=C(O)C(F)(F)F] | 2-[bis(2- hydroxyethyl)amino]- 1,4,5,6- tetrahydropyrimidine- 5-carboxylic acid;2,2,2- trifluoroacetic acid | diethanolamine | 228.0 |
| 89 | 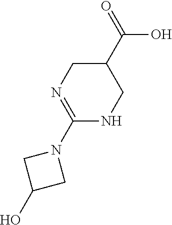 [SMILES: O=C(O)C1CN=C(N2CC(O)C2)NC1] | 2-(3-hydroxyazetidin- 1-yl)-1,4,5,6- tetrahydropyrimidine- 5-carboxylic acid | azetidin-3-ol | 200.1 |
| 90 | 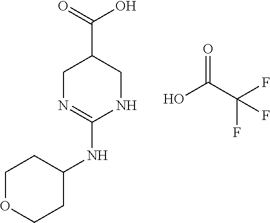 [SMILES: O=C(O)C1CN=C(NC2CCOCC2)NC1 ; O=C(O)C(F)(F)F] | 2-(tetrahydropyran-4- ylamino)-1,4,5,6- tetrahydropyrimidine- 5-carboxylic acid;2,2,2- trifluoroacetic acid | tetrahydro-2H- pyran-4-amine [38041-19-9] | 228.1 |
| 91 | 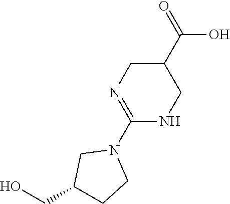 [SMILES: O=C(O)C1CN=C(N2CC[C@H](CO)C2)NC1] | 2-[(3S)-3- (hydroxymethyl)pyrrolidin- 1-yl]-1,4,5,6- tetrahydropyrimidine- 5-carboxylic acid | (S)-pyrrolidin-3- ylmethanol [110013-19-9] | 228.1 |
| 92 | 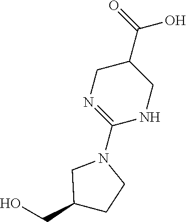 [SMILES: O=C(O)C1CN=C(N2CC[C@@H](CO)C2)NC1] | 2-[(3R)-3- (hydroxymethyl)pyrrolidin- 1-yl]-1,4,5,6- tetrahydropyrimidine- 5-carboxylic acid | (R)-pyrrolidin-3- ylmethanol [110013-18-8] | 228.1 |
| 93 | 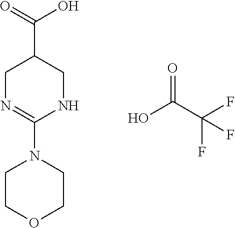 [SMILES: O=C(O)C1CN=C(N2CCOCC2)NC1 ; O=C(O)C(F)(F)F] | 2-morpholino-1,4,5,6- tetrahydropyrimidine- 5-carboxylic acid;2,2,2- trifluoroacetic acid | morpholine | 214.3 |
| 94 | 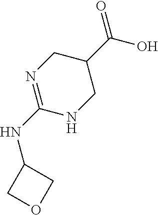 [SMILES: O=C(O)C1CN=C(NC2COC2)NC1] | 2-(oxetan-3-ylamino)- 1,4,5,6- tetrahydropyrimidine- 5-carboxylic acid | 3-oxetanamine [21635-88-1] | 200.1 |
| 95 | 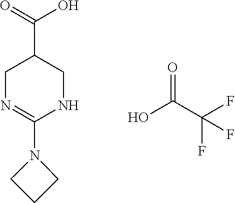 [SMILES: O=C(O)C1CN=C(N2CCC2)NC1 ; O=C(O)C(F)(F)F] | 2-(azetidin-1-yl)- 1,4,5,6- tetrahydropyrimidine- 5-carboxylic acid;2,2,2- trifluoroacetic acid | Azetidine [503-29- 7] | 184.1 |
| 96 | 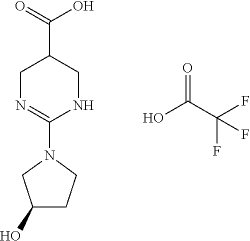 [SMILES: O=C(O)C1CN=C(N2CC[C@@H](O)C2)NC1 ; O=C(O)C(F)(F)F] | 2-[(3R)-3- hydroxypyrrolidin-1- yl]-1,4,5,6- tetrahydropyrimidine- 5-carboxylic acid;2,2,2- trifluoroacetic acid | (R)-pyrrolidin-3-ol [2799-21-5] | 214.0 |
| 97 | 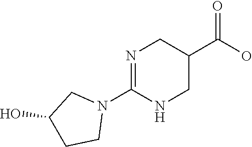 [SMILES: O=C(O)C1CN=C(N2CC[C@H](O)C2)NC1] | 2-[(3S)-3- hydroxypyrrolidin-1- yl]-1,4,5,6- tetrahydropyrimidine- 5-carboxylic acid | (S)-pyrrolidin-3-ol [100243-39-8] | 214.0 |
| 98 | 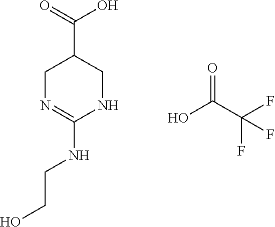 [SMILES: O=C(O)C1CN=C(NCCO)NC1 ; O=C(O)C(F)(F)F] | 2-(2- hydroxyethylamino)- 1,4,5,6- tetrahydropyrimidine- 5-carboxylic acid;2,2,2- trifluoroacetic acid | 2-aminoethan-1-ol [141-43-5] | 188.1 |
| 99 | 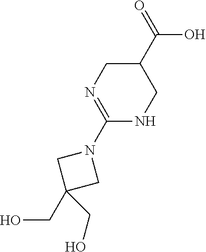 [SMILES: O=C(O)C1CN=C(N2CC(CO)(CO)C2)NC1] | 2-[3,3- bis(hydroxymethyl) azetidin-1-yl]-1,4,5,6- tetrahydropyrimidine- 5-carboxylic acid | [3-(hydroxymethyl) azetidin-3- yl]methanol; hydrochloride [1016232- 92-0] | 244.2 |
| 100 | 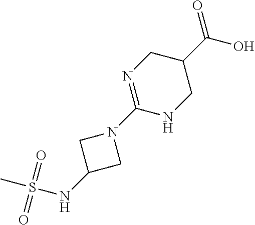 [SMILES: CS(=O)(=O)NC1CN(C2=NCC(C(=O)O)CN2)C1] | 2-[3- (methanesulfonamido) azetidin-1-yl]- 1,4,5,6- tetrahydropyrimidine- 5-carboxylic acid | N-(azetidin-3- yl)methanesulfonamide; hydrochloride [1239205-33-4] | 277.1 |
| 101 | 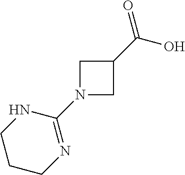 [SMILES: O=C(O)C1CN(C2=NCCCN2)C1] | 1-(1,4,5,6- tetrahydropyrimidin- 2-yl)azetidine-3- carboxylic acid | 2- methylsulfonylpyrimidine [14161-09-2], azetidine-3- carboxylic acid [36476-78-5] | 184.2 |
| 102 | 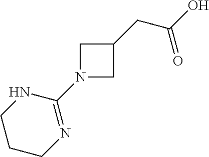 [SMILES: O=C(O)CC1CN(C2=NCCCN2)C1] | 2-[1-(1,4,5,6- tetrahydropyrimidin-2- yl)azetidin-3-yl]acetic acid | 2-methylsulphonyl pyrimidine [14161-09-2], ethyl 2-(azetidin-3- yl)acetate;hydrochloride | 198.1 |
| 103 | 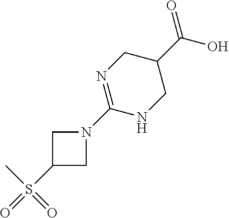 [SMILES: CS(=O)(=O)C1CN(C2=NCC(C(=O)O)CN2)C1] | 2-(3- methylsulphonylazetidin- 1-yl)-1,4,5,6- tetrahydropyrimidine- 5-carboxylic acid | 3-methylsulphonyl azetidine; hydrochloride [1400764-60-4] | 262.1 |
| 104 | 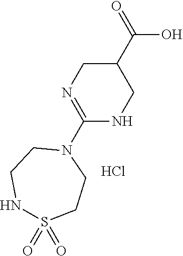 [SMILES: O=C(O)C1CN=C(N2CCNS(=O)(=O)CC2)NC1 ; Cl] | 2-(1,1-dioxo-1,2,5- thiadiazepan-5-yl)- 1,4,5,6- tetrahydropyrimidine- 5-carboxylic acid;hydrochloride | 1,2,5- thiadiazepane 1,1- dioxide [410545-38-9] | 277.0 |
| 105 | 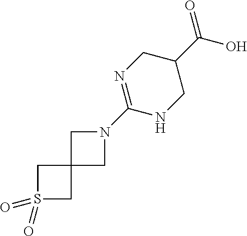 [SMILES: O=C(O)C1CN=C(N2CC3(C2)CS(=O)(=O)C3)NC1] | 2-(2,2-dioxo-2λ6-thia- 6-azaspiro[3.3]heptan- 6-yl)-1,4,5,6- tetrahydropyrimidine- 5-carboxylic acid | 2λ6-thia-6- azaspiro[3.3]heptane 2,2-dioxide; hydrochloride [1427388-39-3] | 274.1 |
| 106 | 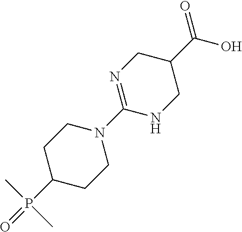 [SMILES: CP(C)(=O)C1CCN(C2=NCC(C(=O)O)CN2)CC1] | 2-(4- dimethylphosphoryl-1- piperidyl)-1,4,5,6- tetrahydropyrimidine- 5-carboxylic acid | 4-dimethyl phosphorylpiperidine; hydrochloride [2940953-08-0] | 288.1 |
| 107 | 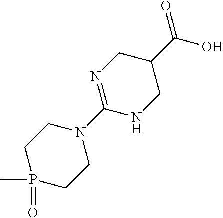 [SMILES: CP1(=O)CCN(C2=NCC(C(=O)O)CN2)CC1] | 2-(4-methyl-4-oxo- 1,4λ5-azaphosphinan- 1-yl)-1,4,5,6- tetrahydropyrimidine- 5-carboxylic acid | 4-methyl-1,4λ5- azaphosphinane 4- oxide;hydrochloride [945459-80-3] | 260.1 |
| 108 | 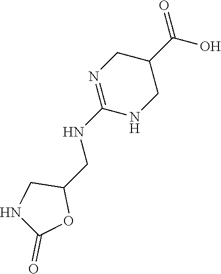 [SMILES: O=C1NCC(CNC2=NCC(C(=O)O)CN2)O1] | 2-[(2-oxooxazolidin-5- yl)methylamino]- 1,4,5,6- tetrahydropyrimidine- 5-carboxylic acid | 5- (aminomethyl) oxazolidin-2- one;hydrochloride | 243.0 |
| 109 | 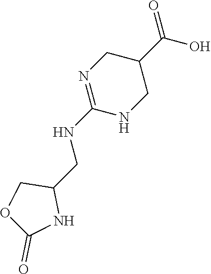 [SMILES: O=C1NC(CNC2=NCC(C(=O)O)CN2)CO1] | 2-[(2-oxooxazolidin-4- yl)methylamino]- 1,4,5,6- tetrahydropyrimidine- 5-carboxylic acid | 4- (aminomethyl) oxazolidin-2- one;hydrochloride [1638763-83-3] | 243.1 |
| 110 | 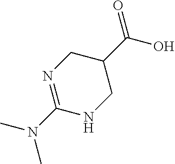 | 2-(dimethylamino)- 1,4,5,6- tetrahydropyrimidine- 5-carboxylic acid | Dimethylamine; hydrochloride | 172.1 |

### Intermediate 111

### 2-(1,1-dioxo-1,4-thiazinan-4-yl)-1,4,5,6-tetrahydropyrimidine-5-carboxylic acid

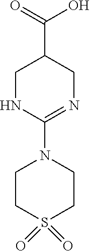 [SMILES: O=C(O)C1CN=C(N2CCS(=O)(=O)CC2)NC1]

### Step 1: 2-(1,1-dioxo-1,4-thiazinan-4-yl)pyrimidine-5-carboxylic acid

To a solution of 2-chloropyrimidine-5-carboxylic acid (200 mg, 1.26 mmol, 1.0 eq) in NMP (5 mL) was added potassium carbonate (349 mg, 2.52 mmol, 2.0 eq) and thiomorpholine 1,1-dioxide (256 mg, 1.89 mmol, 1.5 eq). The mixture was stirred at 100° C. for 16 h. The mixture was diluted with water 3 mL and the pH adjusted to 3 with 1M HCl. The resulting suspension was filtered, the solid washed with water (1 mL×2) and air-dried to afford the title compound (250.0 mg, 0.97 mmol, 77.03% yield) MS: 258.0, [M+H]+, ESI+

### Step 2: 2-(1,1-dioxo-1,4-thiazinan-4-yl)-1,4,5,6-tetrahydropyrimidine-5-carboxylic acid

To a solution of 2-(1,1-dioxo-1,4-thiazinan-4-yl)pyrimidine-5-carboxylic acid (250.0 mg, 0.97 mmol, 1.0 eq) in water (15 mL) was added Pd/SiO2 (250.0 mg) and 1M HCl (1.94 mL, 1.94 mmol, 2.0 eq) at 25° C. under N₂. The reaction mixture was stirred at 25° C. for 16 h under H₂ atmosphere. The mixture was filtered and filtrate was concentrated in vacuo afford the title compound (200 mg, 0.77 mmol, 78.83% yield). MS: 262.0, [M+H]+, ESI+)

The following compounds were prepared in analogy to the procedure above

| Int | Structure | Name | Reagents | MS |
|---|---|---|---|---|
| 112 | 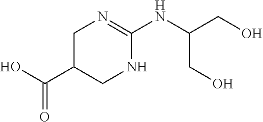 [SMILES: O=C(O)C1CN=C(NC(CO)CO)NC1] | 2-[2-hydroxy-1- (hydroxymethyl)ethyl] amino]-1,4,5,6- tetrahydropyrimidine- 5-carboxylic acid | 2-amino-1,3- propanediol, step 1 in DMF at 80° C. | 218.0, [M + H]+, ESI+ |
| 113 | 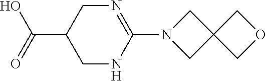 [SMILES: O=C(O)C1CN=C(N2CC3(COC3)C2)NC1] | 2-(2-oxa-6- azaspiro[3.3]heptan- 6-yl)-1,4,5,6- tetrahydropyrimidine- 5-carboxylic acid | 2-oxa-6-azaspiro [3.3]heptane, step 1 in MeCN at 80° C., step 2 TFA instead of HCl | 226.1, [M + H]+, ESI+ |

### Intermediate 114

### 2-(2-methylsulphonylethylamino)-1,4,5,6-tetrahydropyrimidine-5-carboxylic acid

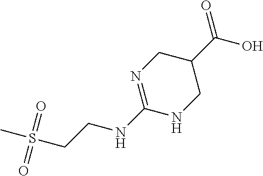 [SMILES: CS(=O)(=O)CCNC1=NCC(C(=O)O)CN1]

### Step 1: methyl 2-(2-methylsulphonylethylamino)pyrimidine-5-carboxylate

To a mixture of 2-aminoethylmethylsulphone hydrochloride (462 mg, 2.9 mmol, 1.0 eq) and K₂CO₃ (1.2 g, 8.69 mmol, 3.0 eq) in MeCN (10 mL) was added methyl 2-chloropyrimidine-5-carboxylate (0.5 g, 2.9 mmol, 1.0 eq). The mixture was then stirred at 80° C. for 12 h. The mixture was diluted with H₂O (15 mL), and a white precipitate was formed. The mixture was filtered. The filtrate was washed with PE (10 mL) and dried to afford the title compound (680 mg, 2.62 mmol, 90% yield). MS: m/z=260.2 [M+H]+, ESI pos.

### Step 2: 2-(2-methylsulphonylethylamino)pyrimidine-5-carboxylic acid

To a solution of methyl 2-(2-methylsulphonylethylamino)pyrimidine-5-carboxylate (680 mg, 2.62 mmol, 1.0 eq) in MeOH (8 mL) and H₂O (2 mL) was added LiOH·H₂O (132 mg, 3.15 mmol, 1.2 eq). The mixture was then stirred at 30° C. for 1 h. The mixture was concentrated under reduced pressure to afford the title compound (643 mg, 2.62 mmol, 100% yield). MS: m/z=246.1 [M+H]+, ESI pos.

### Step 3: 2-(2-methylsulphonylethylamino)-1,4,5,6-tetrahydropyrimidine-5-carboxylic acid

To a solution of 2-(2-methylsulphonylethylamino)pyrimidine-5-carboxylic acid (643 mg, 2.62 mmol, 1.0 eq) in MeOH (12 mL) were added HCl (3.0 mL, 1 M) and Pd/SiO₂ (500 mg, 1.0 eq) under N₂. The mixture was then stirred at 35° C. for 12 h under H₂ (15 psi). The mixture was diluted with MeOH (40 mL) and filtered. The filtrate was concentrated under reduced pressure to afford the title compound (644 mg, 2.58 mmol, 98% yield) as yellow oil. MS: m/z=250.0 [M+H]+, ESI pos.

The following compounds were prepared in analogy to the procedure above

| Int | Structure | Name | Reagents | MS [M + H]+ |
|---|---|---|---|---|
| 115 | 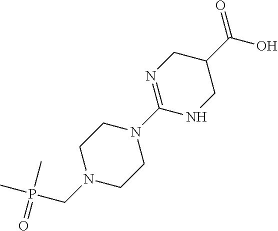 [SMILES: CP(C)(=O)CN1CCN(C2=NCC(C(=O)O)CN2)CC1] | 2-[4- (dimethylphosphoryl- methyl)piperazin-1-yl]- 1,4,5,6- tetrahydropyrimidine- 5-carboxylic acid | 1- (dimethyl- phosphorylmethyl) piperazine; hydrochloride [2445790-96-3] | 303.1 |
| 116 | 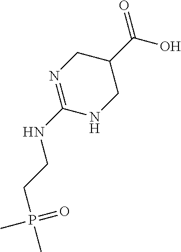 [SMILES: CP(C)(=O)CCNC1=NCC(C(=O)O)CN1] | 2-(2- dimethylphosphoryl- ethylamino)-1,4,5,6- tetrahydropyrimidine- 5-carboxylic acid | 2- dimethyl- phosphorylethanamine; hydrochloride [1003315-34-1] | 248.2 |
| 117 | 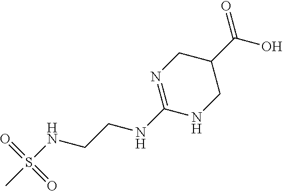 [SMILES: CS(=O)(=O)NCCNC1=NCC(C(=O)O)CN1] | 2-[2- (methanesulfonamido) ethylamino]-1,4,5,6- tetrahydropyrimidine- 5-carboxylic acid | N-(2- aminoethyl) methanesulfonamide; hydrochlorid [202197-61-3]e | 264.9 |

### Intermediate 118

### 2-[tert-butoxycarbonyl(oxetan-3-ylmethyl)amino]-1,4,5,6-tetrahydropyrimidine-5-carboxylic acid

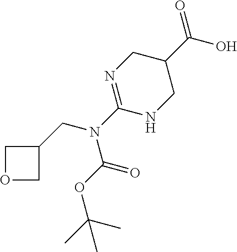 [SMILES: CC(C)(C)OC(=O)N(CC1COC1)C1=NCC(C(=O)O)CN1]

### Step 1: methyl 2-(oxetan-3-ylmethylamino)pyrimidine-5-carboxylate

A mixture of methyl 2-chloropyrimidine-5-carboxylate (2 g, 11.59 mmol, 1.0 eq), 3-oxetanemethanamine (1 g, 11.48 mmol, 0.99 eq) and K₂CO₃ (1.6 g, 11.59 mmol, 1.0 eq) in MeCN (25 mL) was stirred at 25° C. for 12 h. The mixture was poured into H₂O (100 mL) and extracted with EtOAc (3×50 mL). The combined organic phase was washed with brine (3×30 mL), dried with Na₂SO₄, filtered and concentrated under reduced pressure. The residue was purified by column chromatography (SiO₂, PE:EtOAc 2:1 to 1:1) to afford the title compound (1.85 g, 8.29 mmol, 58% yield). MS: m/z=224.0 [M+H]+, ESI pos.

### Step 2: methyl 2-[tert-butoxycarbonyl(oxetan-3-ylmethyl)amino]pyrimidine-5-carboxylate

To a solution of methyl 2-(oxetan-3-ylmethylamino)pyrimidine-5-carboxylate (1.85 g, 8.29 mmol, 1.0 eq), 4-dimethylaminopyridine (0.1 g, 0.83 mmol, 0.1 eq) and triethylamine (2.31 mL, 16.57 mmol, 2.0 eq) in THF (25 mL) was added Boc₂O (4.52 g, 20.72 mmol, 2.5 eq). The mixture was then stirred at 25° C. for 16. The mixture was poured into H₂O (100 mL) and extracted with EtOAc (3×50 mL). The combined organic phase was washed with brine (3×30 mL), dried with Na₂SO₄, filtered and concentrated under reduced pressure. The residue was purified by column chromatography (SiO₂, PE:EtOAc 3:1 to 2:1) to afford the title compound (2.57 g, 7.95 mmol, 91% yield) as yellow oil. 1H NMR (400 MHz, CDCl₃) δ 9.11 (s, 2H), 4.77-4.70 (m, 2H), 4.56 (t, J=6.4 Hz, 2H), 4.34 (d, J=6.8 Hz, 2H), 3.95 (s, 3H), 3.46-3.36 (m, 1H), 1.55 (s, 9H) ppm.

### Step 3: methyl 2-[tert-butoxycarbonyl(oxetan-3-ylmethyl)amino]-1,4,5,6-tetrahydropyrimidine-5-carboxylate

To a solution of methyl 2-[tert-butoxycarbonyl(oxetan-3-ylmethyl)amino]pyrimidine-5-carboxylate (1.5 g, 4.64 mmol, 1.0 eq) and 1,1,2-trichloroethane (0.62 g, 4.64 mmol, 1.0 eq) in MeOH (75 mL) and H₂O (30 mL) was added Pd/SiO₂ (1.5 g, 1.0 eq) under N₂. The mixture was then stirred at 25° C. for 16 h under H₂ (15 psi). The mixture was filtered, and the filtrate was concentrated under reduced pressure. The residue was purified by reverse phase HPLC (H₂O (0.1% EtOAc)-MeCN]; 12-25% B) to afford the title compound (300 mg, 0.92 mmol, 20% yield) as a white gum. MS: m/z=328.3 [M+H]+, ESI pos.

### Step 4: 2-[tert-butoxycarbonyl(oxetan-3-ylmethyl)amino]-1,4,5,6-tetrahydropyrimidine-5-carboxylic acid

To a stirred solution of methyl 2-[tert-butoxycarbonyl(oxetan-3-ylmethyl)amino]-1,4,5,6-tetrahydropyrimidine-5-carboxylate (90 mg, 0.27 mmol, 1.0 eq) in THF (4 mL) and H₂O (2 mL) was added LiOH·H₂O (11 mg, 0.27 mmol, 1.0 eq) at 20° C. The solution was then stirred at 20° C. for 2 h. The reaction mixture was concentrated under reduced pressure to afford crude title compound (86 mg, 0.27 mmol, 99% yield). MS: m/z=314.1 [M+H]+, ESI pos.

### Intermediate 119

### 2-(methylsulphonylmethylamino)-1,4,5,6-tetrahydropyrimidine-5-carboxylic acid

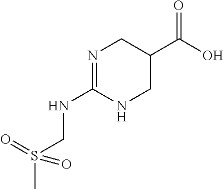

### Step 1: methyl 2-(tert-butoxycarbonylamino)pyrimidine-5-carboxylate

To a solution of methyl 2-aminopyrimidine-5-carboxylate (3 g, 19.59 mmol, 1.0 eq), DMAP (1 g, 8.19 mmol, 0.42 eq) and triethylamine (10.92 mL, 78.36 mmol, 4.0 eq) in DMSO (100 mL) was added Boc₂O (5.13 g, 23.51 mmol, 1.2 eq) at 0° C. The mixture was then stirred at 80° C. for 16 h. The mixture was poured into H₂O (200 mL) and extracted with EtOAc (3×200 mL). The combined organic phase was washed with brine (100 mL×3), dried over Na₂SO₄, filtered, and concentrated under reduced pressure. The residue was purified by reverse phase HPLC (H₂O (0.1% FA)-MeCN]; 50-70% B) to afford the title compound (930 mg, 3.67 mmol, 17% yield). MS: m/z=276.1 [M+Na]+, ESI pos.

### Step 2: methyl 2-[tert-butoxycarbonyl(methylsulfanylmethyl)amino]pyrimidine-5-carboxylate

To a solution of methyl 2-(tert-butoxycarbonylamino)pyrimidine-5-carboxylate (930 mg, 3.67 mmol, 1.0 eq) in CH₂Cl₂ (10 mL) was added tert-butylimino-tri (pyrrolidino)phosphorane (1.6 g, 5.12 mmol, 1.39 eq) and chloromethyl methyl sulphide (0.53 g, 5.51 mmol, 1.5 eq) at 25° C. The mixture was then stirred at 50° C. for 16 h. The mixture was purified by silica column (25% EtOAc in PE) to afford the title compound (530 mg, 1.69 mmol, 45% yield). MS: m/z=649.3 [2M+Na]+, ESI pos.

### Step 3: 2-[tert-butoxycarbonyl(methylsulfanylmethyl)amino]pyrimidine-5-carboxylic acid

To a solution of methyl 2-[tert-butoxycarbonyl(methylsulfanylmethyl)amino]pyrimidine-5-carboxylate (50 mg, 0.16 mmol, 1.0 eq) in THF (2 mL) and H₂O (0.5 mL) was added LiOH·H₂O (7 mg, 0.18 mmol, 1.1 eq) at 20° C. The mixture was then stirred at 20° C. for 16 h. The mixture was purified by reverse phase HPLC (0.1% FA in H₂O-MeCN, B %=50%-80%) to afford the title compound (38 mg, 0.13 mmol, 74% yield). MS: m/z=322.0 [M+Na]+, ESI pos.

### Step 4: 2-[tert-butoxycarbonyl(methylsulphonylmethyl)amino]pyrimidine-5-carboxylic acid

To a solution of 2-[tert-butoxycarbonyl(methylsulfanylmethyl)amino]pyrimidine-5-carboxylic acid (560 mg, 1.87 mmol, 1.0 eq) in CH₂Cl₂ (10 mL) was added 3-chlorobenzenecarboperoxoic acid (949 mg, 4.68 mmol, 2.5 eq). The mixture was then stirred at 20° C. for 2 h. The mixture was concentrated under reduced pressure. The residue was purified by reverse phase HPLC (0.1% FA in H₂O-MeCN, 50-80% B) to afford the title compound (280 mg, 0.85 mmol, 44% yield). MS: m/z=353.9 [M+Na]+, ESI pos.

### Step 5: 2-(methylsulphonylmethylamino)-1,4,5,6-tetrahydropyrimidine-5-carboxylic acid

To a solution of 2-[tert-butoxycarbonyl(methylsulphonylmethyl)amino]pyrimidine-5-carboxylic acid (280 mg, 0.85 mmol, 1.0 eq) in MeOH (50 mL) and H₂O (20 mL) was added HCl/H₂O (2.0 mL, 1 M) and Pd/SiO₂ (200 mg, 5%) under N₂. The reaction mixture was then stirred at 25° C. for 16 h under H₂ (in balloon). The mixture was filtered and concentrated under reduced pressure to afford the title compound (130 mg, 0.55 mmol, 65% yield). MS: m/z=236.1 [M+H]+, ESI pos.

### Intermediate 120

### 6-methylsulfanyl-2-oxa-5,7-diazaspiro[3.4]oct-5-ene

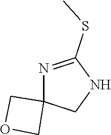

### Step 1: 2-oxa-5,7-diazaspiro[3.4]oct-5-ene-6-thiol

To a solution of 3-(aminomethyl) oxetan-3-amine;oxalic acid (200 mg, 1.04 mmol, 1.0 eq) in CH₂Cl₂ (15 mL) was added N,N-diisopropylethylamine (0.36 mL, 2.08 mmol, 2.0 eq) and 1,1′-thiocarbonyldiimidazole (204 mg, 1.14 mmol, 1.1 eq). The solution was then stirred at 25° C. for 12 h. The mixture was filtered and washed with PE (5 mL), the filter cake was collected to afford the title compound (200 mg, 1.39 mmol, 85% yield). 1H NMR (400 MHz, DMSO-d6) δ 9.03 (s, 1H), 8.22 (s, 1H), 4.64 (d, J=7.2 Hz, 2H), 4.55 (d, J=6.8 Hz, 2H), 3.86 (s, 2H) ppm.

### Step 2: 6-methylsulfanyl-2-oxa-5,7-diazaspiro[3.4]oct-5-ene

To a solution of 2-oxa-5,7-diazaspiro[3.4]oct-5-ene-6-thiol (150 mg, 0.67 mmol, 1.0 eq) in Ethanol (2 mL) was added iodomethane (94.5 mg, 0.67 mmol, 1.0 eq). The mixture was then stirred at 80° C. for 2 h. The mixture was concentrated under reduced pressure to afford the title compound (87 mg, 0.55 mmol, 83% yield). MS: m/z=159.1 [M+H]+, ESI pos.

### Intermediate 121

### (1r,2R,3S)-2,3-bis(((tert-butoxycarbonyl)amino)methyl)cyclopropane-1-carboxylic acid

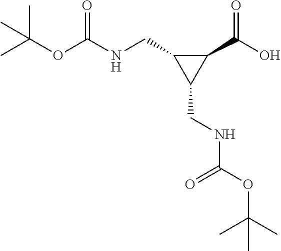 [SMILES: CC(C)(C)OC(=O)NC[C@@H]1[C@H](CNC(=O)OC(C)(C)C)[C@H]1C(=O)O]

### Step 1: methyl (1r,2R,3S)-2,3-bis(hydroxymethyl)cyclopropane-1-carboxylate

A solution of ethyl (1R,7S)-3,5-dioxabicyclo[5.1.0]octane-8-carboxylate ([81056-10-2], 1.54 g, 8.27 mmol, 1.0 eq) in HCl/MeOH (20 mL, 0.5 M) was stirred at 70° C. for 2 h. The mixture was concentrated under reduced pressure to give a residue, which was diluted with MeOH (3 mL) and neutralized with sodium bicarbonate. The mixture was stirred for 0.5 h, filtered and concentrated under reduced pressure. The residue was purified by column chromatography (SiO₂, PE:EtOAc=1:0 to 1:3) to afford the title compound (490 mg, 3.06 mmol, 37% yield) as colorless oil. 1H NMR (400 MHz, CDCl₃) δ 4.17-4.07 (m, 2H), 3.68 (s, 3H), 3.44-3.35 (m, 2H), 2.52-2.38 (m, 2H), 2.03-1.93 (m, 2H), 1.54 (t, J=4.4 Hz, 1H) ppm.

### Step 2: methyl (1s,2R,3S)-2,3-bis(chloromethyl)cyclopropane-1-carboxylate

To a solution of methyl (1r,2R,3S)-2,3-bis(hydroxymethyl)cyclopropane-1-carboxylate (700 mg, 4.37 mmol, 1.0 eq) in CH₂Cl₂ (10 mL) was added pyridine (0.04 mL, 0.44 mmol, 0.1 eq) and thionyl chloride (0.63 mL, 8.74 mmol, 2.0 eq) at 0° C. The mixture was warmed to 20° C. and stirred for 12 h. The mixture was concentrated under reduced pressure. The residue was purified by column chromatography (SiO₂, PE:EtOAc=1:0 to 5:1) to afford the title compound (530 mg, 2.69 mmol, 62% yield) as colorless oil. 1H NMR (400 MHz, CDCl₃) δ 3.72 (s, 3H), 3.69-3.59 (m, 4H), 2.19-2.10 (m, 2H), 1.73 (t, J=4.8 Hz, 1H) ppm.

### Step 3: methyl (1r,2R,3S)-2,3-bis(azidomethyl)cyclopropane-1-carboxylate

To a solution of methyl (1s,2R,3S)-2,3-bis(chloromethyl)cyclopropane-1-carboxylate (640 mg, 3.25 mmol, 1.0 eq) in DMF (10 mL) was added azidotrimethylsilane (1.08 mL, 8.12 mmol, 2.5 eq) and TBAF (9.74 mL, 9.74 mmol, 3.0 eq). The mixture was stirred at 70° C. for 2 h. The reaction mixture was poured into H₂O (30 mL) and extracted with EtOAc (3×30 mL). The combined organic phases were washed by brine (45 mL×2), dried over Na₂SO₄, filtered and concentrated under reduced pressure. The residue was purified by column chromatography (SiO₂, PE:EtOAc=20:1 to 5:1) to afford the title compound (584 mg, 2.78 mmol, 86% yield) as colorless oil. 1H NMR (400 MHz, CDCl₃) δ 3.72 (s, 3H), 3.47 (dd, J=6.4, 13.2 Hz, 2H), 3.35 (dd, J=6.8, 13.2 Hz, 2H), 1.98-1.88 (m, 2H), 1.65 (t, J=4.8 Hz, 1H) ppm.

### Step 4: methyl (1r,2R,3S)-2,3-bis(((tert-butoxycarbonyl)amino)methyl)cyclopropane-1-carboxylate

To a solution of methyl (1r,2R,3S)-2,3-bis(azidomethyl)cyclopropane-1-carboxylate (145 mg, 0.69 mmol, 1.0 eq) in THF (2 mL) and H₂O (2 mL) was added triphenylphosphine (452 mg, 1.72 mmol, 2.5 eq) at 0° C. The mixture was then warmed to 20° C. and stirred for 12 h. The mixture was concentrated under reduced pressure. The residue was diluted with THF (1 mL) and H₂O (1 mL), sodium hydrogen carbonate (173 mg, 2.07 mmol, 3.0 eq) and Boc₂O (451 mg, 2.07 mmol, 3.0 eq) was added to the mixture and stirred at 25° C. for 1 h. The reaction mixture was poured into H₂O (20 mL) and extracted with EtOAc (3×20 mL). The combined organic phases were washed by brine (2×30 mL), dried over Na₂SO₄, filtered and concentrated under reduced pressure. The residue was purified by column chromatography (SiO₂, PE:EtOAc=20:1 to 3:1) to afford the title compound (117 mg, 0.33 mmol, 47% yield). 1H NMR (400 MHz, CDCl₃) δ 5.22 (s, 2H), 3.67 (s, 3H), 3.58-3.38 (m, 2H), 3.14-2.96 (m, 2H), 1.78-1.70 (m, 2H), 1.45 (s, 18H), 1.38-1.31 (m, 1H) ppm.

### Step 6: (1r,2R,3S)-2,3-bis(((tert-butoxycarbonyl)amino)methyl)cyclopropane-1-carboxylic acid

To a solution of methyl (1r,2R,3S)-2,3-bis(((tert-butoxycarbonyl)amino)methyl)cyclopropane-1-carboxylate (117 mg, 0.33 mmol, 1.0 eq) in MeOH (2 mL) was added LiOH·H₂O (13.7 mg, 0.33 mmol, 1.0 eq) and stirred at 25° C. for 1 h. The mixture was concentrated under reduced pressure. The residue was resolved into H₂O (20 mL) and adjusted to pH 4 with 1M HCl aqueous solution. The mixture extracted with EtOAc (3×30 mL). The combined organic phases were washed by brine (2×40 mL), dried over Na₂SO₄, filtered and concentrated under reduced pressure to afford the title compound (105 mg, 0.3 mmol, 93% yield). 1H NMR (400 MHz, CDCl₃) δ 5.40 (s, 2H), 3.64-3.40 (m, 2H), 3.40-3.17 (m, 1H), 3.14-2.92 (m, 2H), 1.81-1.71 (m, 2H), 1.45 (s, 18H), 1.35-1.28 (m, 1H) ppm.

### Intermediate 122

### [1-(2-oxa-6,8-diazaspiro[3.5]non-6-en-7-yl)-4-piperidyl]-piperazin-1-yl-methanone

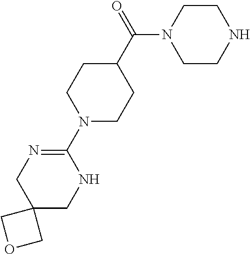 [SMILES: O=C(C1CCN(C2=NCC3(CN2)COC3)CC1)N1CCNCC1]

### Step 1: 2-oxa-6,8-diazaspiro[3.5]non-6-ene-7-thiol

To a solution of [3-(aminomethyl) oxetan-3-yl]methanamine (1 g, 8.61 mmol, 1.0 eq) in CH₂Cl₂ (30 mL) was dropwise added 1,1′-thiocarbonyldiimidazole (1.69 g, 9.47 mmol, 1.1 eq) in 5 portions over 0.2 h. The mixture was then stirred at 20° C. for 2 h. The reaction mixture was filtered and the filter cake was collected to afford the title compound (900 mg, 5.69 mmol, 66% yield). MS: m/z=159.1 [M+H]+, ESI pos.

### Step 2: 7-methylsulfanyl-2-oxa-6,8-diazaspiro[3.5]non-6-ene;hydroiodide

To solution of 2-oxa-6,8-diazaspiro[3.5]non-6-ene-7-thiol (0.9 g, 5.69 mmol, 1.0 eq) in EtOH (20 mL) was added iodomethane (1.21 g, 8.53 mmol, 1.5 eq) at 80° C. The mixture was stirred at 80° C. for 2 h. The reaction mixture was filtered and washed with MeOH (50 mL), the filtrate was concentrated under reduced pressure to afford the title compound (1 g, 3.33 mmol, 59% yield).

### Step 3: benzyl 4-[1-(2-oxa-6,8-diazaspiro[3.5]non-6-en-7-yl)piperidine-4-carbonyl]piperazine-1-carboxylate

A solution of benzyl 4-(piperidine-4-carbonyl)piperazine-1-carboxylate;hydrochloride ([1585033-98-2], 1 g, 2.72 mmol, 1.0 eq), triethylamine (1.52 mL, 10.87 mmol, 4.0 eq) and 7-methylsulfanyl-2-oxa-6,8-diazaspiro[3.5]non-6-ene;hydroiodide (0.9 g, 2.99 mmol, 1.1 eq) in DMF (10 mL) was heated to 80° C. and stirred for 40 h. The reaction mixture was purified by Prep-HPLC (Column: Waters Xbridge C18 150×50 mm×10 um; mobile phase: A: H₂O (10 mM NH₄HCO₃); B: MeCN; Gradient: 19-49% B in 11 min) to afford the title compound (170 mg, 0.37 mmol, 14% yield) MS: m/z=456.1 [M+H]+, ESI pos.

### Step 4: [1-(2-oxa-6,8-diazaspiro[3.5]non-6-en-7-yl)-4-piperidyl]-piperazin-1-yl-methanone

To a mixture of Pd/C (50 mg, 1.0 eq) in THF (10 mL) was added benzyl 4-[1-(2-oxa-6,8-diazaspiro[3.5]non-6-en-7-yl)piperidine-4-carbonyl]piperazine-1-carboxylate (90 mg, 0.2 mmol, 1.0 eq) at 25° C. under N₂. The mixture was then stirred at 25° for 1 h under H₂ (20 psi). The mixture was diluted with MeOH (5 mL), filtered and washed with MeOH (3×5 mL), the filtrate was concentrated under reduced pressure to afford the title compound (60 mg, 0.19 mmol, 95% yield) as a colorless gum. MS: m/z=322.2 [M+H]+, ESI pos.

### Intermediate 123

### 1-(6-methyl-2-oxa-6,8-diazaspiro[3.5]non-7-en-7-yl)piperidine-4-carboxylic acid

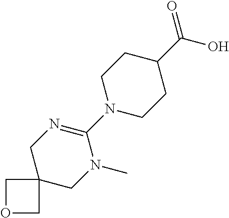 [SMILES: CN1CC2(CN=C1N1CCC(C(=O)O)CC1)COC2]

### Step 1: benzyl 1-(2-oxa-6,8-diazaspiro[3.5]non-6-en-7-yl)piperidine-4-carboxylate

To a solution of benzyl piperidine-4-carboxylate;hydrochloride ([704879-64-1], 1187 mg, 4.64 mmol, 1.0 eq) in DMF (4 mL) were added triethylamine (1.94 mL, 13.93 mmol, 3.0 eq) and 7-methylsulfanyl-2-oxa-6,8-diazaspiro[3.5]non-6-ene (800 mg, 4.64 mmol, 1.0 eq). The mixture was then stirred at 80° C. for 12 h. The mixture was poured into H₂O (20 mL), extracted with EtOAc (2×20 mL). The aqueous phase was concentrated under reduced pressure. The residue was purified by reverse phase HPLC (H₂O-MeCN; 5-30%) to afford the title compound (250 mg, 0.73 mmol, 16% yield). MS: m/z=344.2 [M+H]+, ESI pos.

### Step 2: benzyl 1-(6-methyl-2-oxa-6,8-diazaspiro[3.5]non-7-en-7-yl)piperidine-4-carboxylate

To a mixture of benzyl 1-(2-oxa-6,8-diazaspiro[3.5]non-6-en-7-yl)piperidine-4-carboxylate (150 mg, 0.44 mmol, 1.0 eq) in THF (5 mL) was added sodium hydride (60% in oil)(28 mg, 0.7 mmol, 1.6 eq) at 0° C. The reaction mixture was stirred at 0° C. for 0.5 h under N₂. Iodomethane (124 mg, 0.87 mmol, 2.0 eq) was added to the mixture and the resulting mixture was stirred at 25° C. for another 3.5 h. The mixture was quenched with saturated NH₄Cl aqueous solution (2 mL) slowly, then concentrated under reduced pressure. The residue was purified by reverse phase HPLC (H₂O-MeCN 10-40%) and SFC (column: DAICEL CHIRALPAK IC (250 mm×30 mm, 10 μm); mobile phase: [A: CO₂; B: EtOH (0.1% NH₃H₂O)]; 70-70% B in 3.80 min) to afford the title compound (55 mg, 0.15 mmol, 34% yield). MS: m/z=358.2 [M+H]+, ESI pos.

### Step 3: 1-(6-methyl-2-oxa-6,8-diazaspiro[3.5]non-7-en-7-yl)piperidine-4-carboxylic acid

To a solution of benzyl 1-(6-methyl-2-oxa-6,8-diazaspiro[3.5]non-7-en-7-yl)piperidine-4-carboxylate (45 mg, 0.13 mmol, 1.0 eq) in MeOH (0.5 mL) and H₂O (0.5 mL) was added sodium hydroxide (7 mg, 0.19 mmol, 1.5 eq). The reaction mixture was then stirred at 50° C. for 3 h. The mixture was diluted with H₂O (10 mL), adjusted to pH 7 with HCl aqueous solution (1 N). The resulting mixture was lyophilized to afford the title compound (30 mg, 0.11 mmol, 89% yield). MS: m/z=268.1 [M+H]+, ESI pos.

### Intermediate 124

### [2-(dimethylamino)-1-methyl-5,6-dihydro-4H-pyrimidin-5-yl]-piperazin-1-yl-methanone;hydrochloride

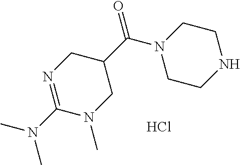 [SMILES: CN(C)C1=NCC(C(=O)N2CCNCC2)CN1C ; Cl]

### Step 1: tert-butyl 4-[2-(dimethylamino)pyrimidine-5-carbonyl]piperazine-1-carboxylate

To a solution of 1-BOC-piperazine (2.23 g, 11.96 mmol, 1.0 eq), 2-(dimethylamino)pyrimidine-5-carboxylic acid ([180283-66-3], 2 g, 11.96 mmol, 1.0 eq), triethylamine (3.34 mL, 23.93 mmol, 2.0 eq) in DMF (20 mL) was added HATU (4.55 g, 11.96 mmol, 1.0 eq). The mixture was then stirred at 25° C. for 0.5 h. The mixture was purified by reverse phase HPLC (0.1% FA in H₂O-MeCN, 70% MeCN) to afford the title compound (2 g, 5.96 mmol, 50% yield). MS: m/z=336.2 [M+H]+, ESI pos.

### Step 2: tert-butyl 4-[2-(dimethylamino)-1,4,5,6-tetrahydropyrimidine-5-carbonyl]piperazine-1-carboxylate

To a solution of tert-butyl 4-[2-(dimethylamino)pyrimidine-5-carbonyl]piperazine-1-carboxylate (2000 mg, 5.96 mmol, 1.0 eq) in MeOH (100 mL)/H₂O (100 mL) were added Pd/SiO₂ (2000 mg, 1.0 eq) and HCl/H₂O (8.94 mL, 1 M) at 25° C. The mixture was then stirred at 25° C. for 16 h under H₂. The reaction mixture was filtered. The filtrate was adjusted to pH=7 with saturated NaHCO₃ solution. The resulting mixture was concentrated under reduced pressure. The residue was triturated in MeCN (100 mL) for 0.5 h. The resulting mixture was filtered and the filtrate was concentrated under reduced pressure to afford the title compound (1300 mg, 3.83 mmol, 44% yield) as yellow oil. MS: m/z=340.2 [M+H]+, ESI pos.

### Step 3: tert-butyl 4-[2-(dimethylamino)-1-methyl-5,6-dihydro-4H-pyrimidine-5-carbonyl]piperazine-1-carboxylate

To a solution of tert-butyl 4-[2-(dimethylamino)-1,4,5,6-tetrahydropyrimidine-5-carbonyl]piperazine-1-carboxylate (500 mg, 1.47 mmol, 1.0 eq) in THF (15 mL) was added sodium hydride (58 mg, 1.47 mmol, 1.0 eq) at 0° C. After 0.5 h, iodomethane (209 mg, 1.47 mmol, 1.0 eq) was added to the mixture. The resulting mixture was then stirred at 25° C. for 16 h. The mixture was concentrated and purified by prep-HPLC (Phenomenex luna C18 150×25 mm×10 um; mobile phase: [H₂O (FA)-MeCN]; 7-37% B, 10 min) to afford the title compound (200.0 mg, 0.57 mmol, 22% yield). MS: m/z=354.1 [M+H]+, ESI pos.

### Step 4: [2-(dimethylamino)-1-methyl-5,6-dihydro-4H-pyrimidin-5-yl]-piperazin-1-yl-methanone;hydrochloride

A solution of tert-butyl 4-[2-(dimethylamino)-1-methyl-5,6-dihydro-4H-pyrimidine-5-carbonyl]piperazine-1-carboxylate (200 mg, 0.57 mmol, 1.0 eq) in HCl/dioxane (2.5 mL, 2 M) was stirred at 25° C. for 0.5 h. The mixture was concentrated under reduced pressure to afford the title compound (150 mg, 0.52 mmol, 91% yield). MS: m/z=254.2 [M+H]+, ESI pos.

### Intermediate 125

### tert-butyl 4-[(4-amino-2-chlorophenyl)sulfonimidoyl]piperazine-1-carboxylate

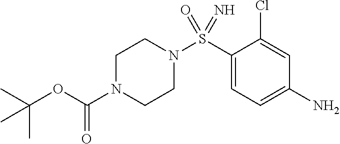 [SMILES: CC(C)(C)OC(=O)N1CCN(S(=N)(=O)c2ccc(N)cc2Cl)CC1]

### Step 1: tert-butyl 4-(2-chloro-4-nitrophenyl)sulfanylpiperazine-1-carboxylate

To a solution of 2-chloro-4-nitrobenzenethiol ([36776-29-1], 5.5 g, 29.01 mmol, 1.0 eq) in CH₂Cl₂ (60 mL) was added N-chlorosuccinimide (3.87 g, 29.01 mmol, 1.0 eq) and stirred at 10° C. for 0.5 h, then a solution of 1-BOC-piperazine (5.4 g, 29.01 mmol, 1.0 eq) and triethylamine (8.09 mL, 58.01 mmol, 2.0 eq) in CH₂Cl₂ (30 mL) was added to the mixture. The resulting mixture was then stirred at 10° C. for 1 h. The mixture was concentrated under reduced pressure. The residue was purified by reverse phase HPLC (H₂O (0.1% FA)-MeCN; B %: 100%) to afford the title compound (6.2 g, 16.58 mmol, 57% yield). MS: m/z=317.9 [M−C4H9+H]+, ESI pos.

### Step 2: tert-butyl 4-[(2-chloro-4-nitrophenyl)sulfonimidoyl]piperazine-1-carboxylate

To a mixture of tert-butyl 4-(2-chloro-4-nitrophenyl)sulfanylpiperazine-1-carboxylate (6.2 g, 16.58 mmol, 1.0 eq) in MeOH (100 mL) were added ammonium carbamate (6.47 g, 82.92 mmol, 5.0 eq) and iodobenzene diacetate (26.71 g, 82.92 mmol, 5.0 eq) at 10° C. The resulting mixture was then stirred at 10° C. for 2 h. The reaction mixture was poured into H₂O (800 mL), extracted with EtOAc (4×300 mL). The combined organic layer was washed with brine (400 mL), dried over Na₂SO₄, filtered and concentrated under reduced pressure. The residue was purified by column chromatography (SiO₂, PE:EtOAc=0:1 to 1:1) to afford the title compound (3.5 g, 8.64 mmol, 52% yield) as brown oil. MS: m/z=405.1 [M+H]+, ESI pos.

### Step 3: tert-butyl 4-[(4-amino-2-chlorophenyl)sulfonimidoyl]piperazine-1-carboxylate

To a mixture of tert-butyl 4-[(2-chloro-4-nitrophenyl)sulfonimidoyl]piperazine-1-carboxylate (1 g, 2.47 mmol, 1.0 eq) in MeOH (10 mL) and THF (30 mL) was added HCl/H₂O (3 mL, 1 M) and Pd/SiO₂ (2 g, 1.0 eq) under N₂. The resulting mixture was then stirred at 45° C. for 1 h under H₂ (1.5 MPa). The reaction mixture was cooled to RT, filtered, the filtrate was concentrated under reduced pressure to afford the title compound (702 mg, 1.87 mmol, 53% yield). MS: m/z=375.1 [M+H]+, ESI pos.

### Intermediate 126

### [2-cyclopropyl-3-fluoro-4-(fluoromethoxy)phenyl]amine

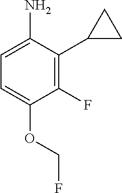 [SMILES: Nc1ccc(OCF)c(F)c1C1CC1]

### Step 1: 3-cyclopropyl-2-fluoro-4-nitrophenol

A mixture of 3-bromo-2-fluoro-4-nitrophenol (320 mg, 1.29 mmol, 1.000 eq), cyclopropylboronic acid (165 mg, 1.93 mmol, 1.500 eq), tricyclohexylphosphine (72 mg, 257.63 μmol, 0.200 eq), palladium (II) acetate (28 mg, 128.81 μmol, 0.100 eq) and tripotassium phosphate (976 mg, 4.51 mmol, 3.500 eq) in toluene (9.6 mL) and H₂O (1.28 mL) was stirred under Ar at 110° C. in a sealed tube. The reaction was concentrated to dryness and the residue was taken up in EtOAc and washed with H₂O and brine, the organic layer dried over MgSO₄, filtered and concentrated under reduced pressure. The residue was purified on SiO₂ using heptane/EtOAc as eluents. The corresponding fractions were combined and concentrated under reduced pressure to afford the title compound (210 mg, 78%) as light-yellow solid. MS: 198.1 [M+H]+, ESI pos

### Step 2: 3-cyclopropyl-2-fluoro-1-(fluoromethoxy)-4-nitrobenzene

To a solution of 3-cyclopropyl-2-fluoro-4-nitrophenol (210 mg, 1. mmol, 1.000 eq) in MeCN (3 mL) were added DIEA (258 mg, 349.7 μL, 2. mmol, 2.000 eq) and fluoro(iodo)methane (320 mg, 135.7 μL, 2. mmol, 2.000 eq). The vessel was sealed and the mixture was heated to 50° C. for 15 h. The reaction mixture was concentrated under reduced pressure and the residue was extracted using EtOAc and deionized H₂O. The organic layer was washed with brine, dried over MgSO₄, filtered and evaporated. The residue as drypack on isolute HM-N was purified on SiO₂ using EtOAc:heptane as eluents. The corresponding fractions were combined and concentrated under reduced pressure to afford the title compound (244 mg, 99%) as light-yellow liquid. MS: m/z=230.1 [M+H]+, ESI pos

### Step 3: [2-cyclopropyl-3-fluoro-4-(fluoromethoxy)phenyl]amine

3-cyclopropyl-2-fluoro-1-(fluoromethoxy)-4-nitrobenzene (244 mg, 0.990 mmol, 1.000 eq) was dissolved in MeOH (5 mL) and palladium on carbon (105 mg, 99.01 μmol, 0.100 eq) was added. The black suspension was 3 times evacuated and backfilled with hydrogen and the reaction stirred for 2 h. Then, the reaction mixture was filtered over diatomaceous filter-aid and the obtained solution concentrated under reduced pressure. The residue was purified on SiO₂ using EtOAc/heptane as eluents. The corresponding fractions were combined and concentrated under reduced pressure to afford the title compound (131 mg, 62%) as brown liquid. MS: 200.1 [M+H]+, ESI pos

The following compound was prepared in analogy to the procedure above

| Int | Structure | Name | Reagents | MS |
|---|---|---|---|---|
| 127 | 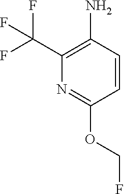 [SMILES: Nc1ccc(OCF)nc1C(F)(F)F] | [6- (fluoromethoxy)-2- (trifluoromethyl)-3- pyridyl]amine | From step 2, using 5-nitro-6- (trifluoromethyl) pyridin-2-ol | m/z = 211.0 [M + H]+, ESI pos |

### Intermediate 128

### [1-(2,2-difluoroethyl)-3-(trifluoromethyl)pyrazol-4-yl]amine

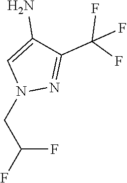 [SMILES: Nc1cn(CC(F)F)nc1C(F)(F)F]

### Step 1: 1-(2,2-difluoroethyl)-4-nitro-3-(trifluoromethyl)pyrazole

To a solution of 4-nitro-3-(trifluoromethyl)-1H-pyrazole (2 g, 11.05 mmol, 1.000 eq) in anhydrous DMF (30 mL) was added K₂CO₃ (3.05 g, 22.09 mmol, 2.000 eq) and 1,1-difluoro-2-iodo-ethane (5.3 g, 2.43 mL, 27.61 mmol, 2.500 eq). The reaction was heated to 70° C. and stirred for 16 h. The reaction was diluted with H₂O and extracted 3 times with EtOAc. The combined organic layers were washed several times with H₂O and brine, dried over MgSO₄, filtered and concentrated under reduced pressure. The residue as drypack on isolute HM-N was purified on SiO₂ using heptane/EtOAc as eluent. The corresponding fractions were concentrated under reduced pressure to afford the title compound (1.6 g, 59%) as light-yellow liquid. MS: m/z=290.1 [M+HCOO]−, ESI neg.

### Step 2: [1-(2,2-difluoroethyl)-3-(trifluoromethyl)pyrazol-4-yl]amine

1-(2,2-difluoroethyl)-4-nitro-3-(trifluoromethyl)pyrazole (1.6 g, 6.53 mmol, 1.000 eq) was dissolved in MeOH (20 mL) and 10% palladium on carbon (694 mg, 652.77 μmol, 0.100 eq) was added. The black suspension was 3 times evacuated and backfilled with hydrogen and the reaction stirred 5 h. The reaction mixture was filtered 2 times through diatomaceous filter-aid and the obtained solution concentrated to afford the title compound (1.26 g, 84.97%) as light brown liquid. MS: m/z=216.0 [M+H]+, ESI pos.

### Intermediate 129

### 5-(tert-butoxycarbonylamino)-1-[2-cyclopropyl-3-fluoro-4-(fluoromethoxy)phenyl]imidazole-4-carboxylic acid

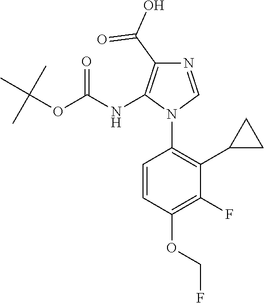 [SMILES: CC(C)(C)OC(=O)Nc1c(C(=O)O)ncn1-c1ccc(OCF)c(F)c1C1CC1]

### Step 1: 5-amino-1-[2-cyclopropyl-3-fluoro-4-(fluoromethoxy)phenyl]imidazole-4-carboxylic acid ethyl ester

To a solution of 2-amino-2-cyano-acetic acid ethyl ester (Intermediate 1, 103.69 mg, 728.31 μmol, 1.200 eq) in MeCN (2 mL) was added a second solution of [2-cyclopropyl-3-fluoro-4-(fluoromethoxy)phenyl]amine (Intermediate 126, 130 mg, 606.93 μmol, 1.000 eq) in MeCN (2 mL). The reaction mixture was stirred at 80° C. for 3 h. Then triethyl orthoformate (110 mg, 123.75 μL, 728.31 μmol, 1.200 eq) was added and the reaction mixture was stirred for another 3 h 50 min at 80° C. The reaction mixture was allowed to cool down and stirred at RT overnight. The reaction mixture was concentrated under reduced pressure, and the residue was dissolved in EtOAc and washed with saturated aqueous NaHCO₃ solution and brine. The aqueous layers were extracted with EtOAc. The combined organic layers were dried over MgSO₄, filtered and concentrated under reduced pressure. The residue was suspended in TBME. Filtration and collection of the solid afforded the title compound (101 mg, 48%) as off-white solid. The mother liquor was concentrated and purified on SiO₂ using heptane/EtOAc as eluent. The corresponding fractions were concentrated under reduced pressure to afford more of the title compound (16.5 mg, 7%) as light-yellow solid. Both materials were combined and used in subsequent steps. MS: 338.2 [M+H]+, ESI pos.

### Step 2: 5-[bis(tert-butoxycarbonyl)amino]-1-[2-cyclopropyl-3-fluoro-4-(fluoromethoxy)phenyl]imidazole-4-carboxylic acid ethyl ester

To a light-yellow solution of 5-amino-1-[2-cyclopropyl-3-fluoro-4-(fluoromethoxy)phenyl]imidazole-4-carboxylic acid ethyl ester (117 mg, 336.45 μmol, 1.000 eq) and DIEA (130 mg, 176.3 μL, 1.01 mmol, 3.000 eq) in CH₂Cl₂ (2 mL) was added (Boc)₂O (183 mg, 195.3 μL, 841.12 μmol, 2.500 eq) and 4-dimethylaminopyridine (8 mg, 67.3 μmol, 0.200 eq) and the reaction stirred 3.5 h. The reaction was diluted with H₂O and extracted 3 times with CH₂Cl₂. The combined organic layers were washed with brine, dried over MgSO₄, filtered and concentrated under reduced pressure. The residue was purified on SiO₂ using heptane/EtOAc as eluents. The corresponding fractions were concentrated under reduced pressure afford the title compound (166 mg, 87%) as light-yellow foam. MS: m/z=538.4 [M+H]+, ESI pos

### Step 3: 5-(tert-butoxycarbonylamino)-1-[2-cyclopropyl-3-fluoro-4-(fluoromethoxy)phenyl]imidazole-4-carboxylic acid

To a mixture of 5-[bis(tert-butoxycarbonyl)amino]-1-[2-cyclopropyl-3-fluoro-4-(fluoromethoxy)phenyl]imidazole-4-carboxylic acid ethyl ester (166 mg, 0.293 mmol, 1.000 eq) in THF (3 mL) and H₂O (1.5 mL) was added 1 M LiOH 1M solution in H₂O (1.47 mL, 1.47 mmol, 5.000 eq) and LiOH·H₂O (123 mg, 2.93 mmol, 10.000 eq). The reaction was heated to 30° C. for 15 h. 25% aqueous HCl (855.7 mg, 713 μL, 5.87 mmol, 20.000 eq) was slowly added (pH between 1-2). The mixture was extracted with EtOAc (3 times). The combined organic layers were washed with brine, dried over MgSO4, filtered and evaporated to afford the title compound (130 mg, 100%) as light-yellow foam. MS: m/z=410.2 [M+H]+, ESI pos

The following compounds were prepared in analogy to the procedure above

| Int | Structure | Name | Reagents | MS |
|---|---|---|---|---|
| 130 | 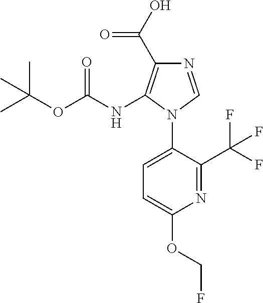 [SMILES: CC(C)(C)OC(=O)Nc1c(C(=O)O)ncn1-c1ccc(OCF)nc1C(F)(F)F] | 5-(tert- butoxycarbonylamino)- 1-[6-(fluoromethoxy)-2- (trifluoromethyl)-3- pyridyl]imidazole-4- carboxylic acid | [6- (fluoromethoxy)- 2- (trifluoromethyl)- 3-pyridyl]amine (Intermediate 127) | m/z = 421.2 [M + H]+, ESI pos |
| 131 | 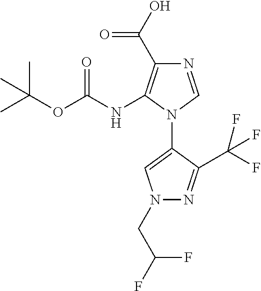 [SMILES: CC(C)(C)OC(=O)Nc1c(C(=O)O)ncn1-c1cn(CC(F)F)nc1C(F)(F)F] | 5-(tert- butoxycarbonylamino)- 1-[1-(2,2-difluoroethyl)- 3- (trifluoromethyl)pyrazol- 4-yl]imidazole-4- carboxylic acid | [1-(2,2- difluoroethyl)-3- (trifluoromethyl) pyrazol-4-yl]amine (Intermediate 128) | m/z = 426.1 [M + H]+, ESI pos |

### Intermediate 132

### 5-(tert-butoxycarbonylamino)-1-[3-fluoro-4-(fluoromethoxy)-2-(trifluoromethyl)phenyl]imidazole-4-carboxylic acid

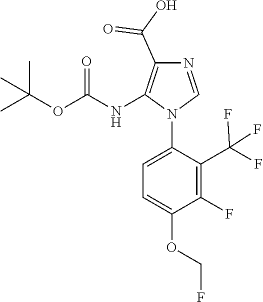 [SMILES: CC(C)(C)OC(=O)Nc1c(C(=O)O)ncn1-c1ccc(OCF)c(F)c1C(F)(F)F]

### Step 1: 5-amino-1-[4-bromo-3-fluoro-2-(trifluoromethyl)phenyl]imidazole-4-carboxylic acid ethyl ester

To a solution of 2-amino-2-cyano-acetic acid ethyl ester (Intermediate 1, 2.24 g, 16.3 mmol, 1.40 eq) in MeCN (15 mL) was added a second solution of [4-bromo-3-fluoro-2-(trifluoromethyl)phenyl]amine (3 g, 12 mmol, 1.0 eq) in MeCN (15 mL). The reaction mixture was stirred at 80° C. for 1 h 50 min. Then triethyl orthoformate (2.46 g, 2.77 mL, 16.3 mmol, 1.40 eq) was added and the reaction mixture was stirred further at 80° C. for 15 h. The reaction mixture was allowed to cool to RT and was then concentrated under reduced pressure. The residue was dissolved in EtOAc and washed with saturated aqueous NaHCO₃ solution and brine. The aqueous layers were extracted with EtOAc. The combined organic layers were dried over MgSO₄, filtered and concentrated under reduced pressure. The residue was suspended in TBME, the solid was filtered and collected. The obtained solid as a dry pack on isolute HM-N adsorbent was purified on SiO₂ using heptane/EtOAc as eluent. The corresponding fractions were combined and concentrated under reduced pressure to afford the title compound (1.02 g, 8%) as off-white solid, which was used without further purification. MS: m/z=396.1, 398.1 [M+H]+, ESI pos

### Step 2: 5-[bis(tert-butoxycarbonyl)amino]-1-[4-bromo-3-fluoro-2-(trifluoromethyl)phenyl]imidazole-4-carboxylic acid ethyl ester

To a yellow suspension of 5-amino-1-[4-bromo-3-fluoro-2-(trifluoromethyl)phenyl]imidazole-4-carboxylic acid ethyl ester (1 g, 883.53 μmol, 1.000 eq) and DIEA (342 mg, 463 μL, 2.65 mmol, 3.000 eq) in CH₂Cl₂ (8 mL) was added (Boc)₂O (482 mg mg, 2.21 mmol, 2.5 eq) and 4-dimethylaminopyridine (21 mg, 176.71 μmol, 0.200 eq) and the reaction stirred at RT for 15 h. The solvent was evaporated and the residue was dissolved in EtOAc and washed with H₂O. The aqueous layer was 2 additional times extracted with EtOAc. The combined organic layers were washed with brine, dried over MgSO₄, filtered and concentrated under reduced pressure. The residue as drypack on isolute HM-N adsorbent was purified on SiO₂ using heptane/EtOAc as eluent. The corresponding fractions were combined and concentrated under reduced pressure to afford the title compound (406 mg, 62%) as a light-yellow foam with a purity of approximately 80%. The material was used in the next step without further purification. MS: 598.4 [M+H]+, ESI pos

### Step 3: [4-[5-[bis(tert-butoxycarbonyl)amino]-4-carbethoxy-imidazol-1-yl]-2-fluoro-3-(trifluoromethyl)phenyl]boronic acid

The reaction was performed in a sealed tube. To a mixture of 5-[bis(tert-butoxycarbonyl)amino]-1-[4-bromo-3-fluoro-2-(trifluoromethyl)phenyl]imidazole-4-carboxylic acid ethyl ester (406 mg, 544.63 μmol, 1.000 eq), potassium acetate (160 mg, 1.63 mmol, 3.000 eq) and bis(pinacolato)diboron (165 mg, 653.55 μmol, 1.200 eq) in a glass tube was added dry 1,2-dimethoxyethane (3.44 g, 3.96 mL, 38.12 mmol, 70.000 eq) and the mixture was flushed with argon. To this 1,1′-bis(diphenylphosphino)ferrocene palladium (ii) chloride CH₂Cl₂ complex (48 mg, 59.91 μmol, 0.110 eq) was added in one portion and the vial sealed. The mixture was then heated to 90° C. for 12 h. The reaction was cooled to RT, diluted with H₂O and extracted 3 times with EtOAc. The combined organic layers were dried over MgSO₄, filtered and concentrated under reduced pressure. The obtained material was purified on SiO₂ using EtOAc/MeOH as eluent. The corresponding fractions were combined and concentrated under reduced pressure to afford the title compound (304 mg, 60%) as light brown foam with an estimated purity of ˜60%. The material was used as is in subsequent steps. MS: m/z=562.3 [M+H]+, ESI pos

### Step 4: 5-[bis(tert-butoxycarbonyl)amino]-1-[3-fluoro-4-hydroxy-2-(trifluoromethyl)phenyl]imidazole-4-carboxylic acid ethyl ester

[4-[5-[bis(tert-butoxycarbonyl)amino]-4-carbethoxy-imidazol-1-yl]-2-fluoro-3-(trifluoromethyl)phenyl]boronic acid (304 mg, 433.29 μmol, 1.000 eq) was dissolved in THF (10.67 mL) and H₂O (4 mL). Then, sodium perborate tetrahydrate (266 mg, 1.73 mmol, 4.000 eq) was added in 4 portions and the reaction stirred at RT 20 h. The reaction was diluted with H₂O and extracted 3 times with EtOAc and 3 times with CH₂Cl₂. The combined organic layers were dried over MgSO₄, filtered and evaporated. The residue was purified on SiO₂ using CH₂Cl₂/(CH₂Cl₂:MeOH 9:1+0.25% NH₄OH) as eluent. The corresponding fractions were concentrated under reduced pressure to afford the title compound (189 mg, 78%) as light-yellow foam. MS: 534.3 [M+H]+, ESI pos

### Step 5: 5-[bis(tert-butoxycarbonyl)amino]-1-[3-fluoro-4-(fluoromethoxy)-2-(trifluoromethyl)phenyl]imidazole-4-carboxylic acid ethyl ester

To a solution of 5-[bis(tert-butoxycarbonyl)amino]-1-[3-fluoro-4-hydroxy-2-(trifluoromethyl)phenyl]imidazole-4-carboxylic acid ethyl ester (211 mg, 367.84 μmol, 1.000 eq) and DIEA (142 mg, 192.7 μL, 1.1 mmol, 3.000 eq) in MeCN (5 mL) in a glass vial was added fluoro(iodo)methane (117 mg, 49.85 μL, 735.67 μmol, 2.000 eq). The tube was sealed and heated to 50° C. for 15 h. The solvent was evaporated and the residue was diluted with EtOAc and H₂O. The aqueous layer was extracted 2 additional times with EtOAc. The combined organic layers were washed with brine, dried over MgSO₄ and evaporated. The residue was purified on SiO₂ using heptane/EtOAc as eluent. The corresponding fractions were obtained to afford the title compound (143 mg, 65%). MS: 566.3 [M+H]+, ESI pos

And to afford 5-(tert-butoxycarbonylamino)-1-[3-fluoro-4-(fluoromethoxy)-2-(trifluoromethyl)phenyl]imidazole-4-carboxylic acid ethyl ester (24 mg; 13%) as light-yellow solid. MS: 466.3 [M+H]+, ESI pos. The combined obtained products were used in the subsequent step

### Step 6: 5-(tert-butoxycarbonylamino)-1-[3-fluoro-4-(fluoromethoxy)-2-(trifluoromethyl)phenyl]imidazole-4-carboxylic acid

To a mixture of 5-[bis(tert-butoxycarbonyl)amino]-1-[3-fluoro-4-(fluoromethoxy)-2-(trifluoromethyl)phenyl]imidazole-4-carboxylic acid ethyl ester (167 mg, 280.55 μmol, 1.000 eq) in THF (3 mL) and H₂O (1 mL) was added 1 M LiOH in H₂O (1.4 mL, 1.4 mmol, 5.000 eq) and LiOH (67 mg, 2.81 mmol, 10.000 eq). The reaction was heated to 40° C. for 15 h. The reaction was cooled to RT. Then, 25% aqueous HCl (695 mg, 579.6 μL, 4.77 mmol, 17.000 eq) was slowly added (pH between 1-2). The mixture was extracted with EtOAc (3 times). The combined organic layers were washed with brine, dried over MgSO₄, filtered and evaporated to afford the title compound (113 mg, 89%) as a light-brown solid. MS: 438.2 [M+H]+, ESI pos

### Intermediate 133

### 5-amino-N-[3-chloro-4-(piperazine-1-carbonyl)phenyl]-1-[2-(difluoromethyl)-3-fluoro-4-(fluoromethoxy)phenyl]imidazole-4-carboxamide

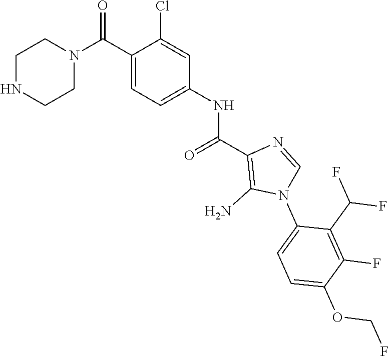 [SMILES: Nc1c(C(=O)Nc2ccc(C(=O)N3CCNCC3)c(Cl)c2)ncn1-c1ccc(OCF)c(F)c1C(F)F]

### Step 1: 5-amino-1-[2-(difluoromethyl)-3-fluoro-4-(fluoromethoxy)phenyl]imidazole-4-carboxylic acid

To sodium hydroxide (1900.4 mg, 47.51 mmol, 5.0 eq) was added to ethyl 5-amino-1-[2-(difluoromethyl)-3-fluoro-4-(fluoromethoxy)phenyl]imidazole-4-carboxylate (Intermediate 3, 3300 mg, 9.5 mmol, 1.0 eq) in IPA (4 mL) and H₂O (2 mL), the resultant mixture was stirred at 50° C. for 2 h. The mixture was adjusted to pH 5-6 with 2 N aqueous HCl and extracted with EtOAc. The reaction mixture was partitioned between EtOAc and H₂O. The aqueous layer was extracted again with EtOAc. The combined organic layer was dried over Na₂SO₄ and concentrated under reduced pressure to afford the title compound (1500 mg, 4.7 mmol, 49% yield). MS: m/z=320.0 [M+H]+, ESI pos

### Step 2: 5-amino-1-[2-(difluoromethyl)-3-fluoro-4-(fluoromethoxy)phenyl]imidazole-4-carbonyl chloride

A solution of 5-amino-1-[2-(difluoromethyl)-3-fluoro-4-(fluoromethoxy)phenyl]imidazole-4-carboxylic acid (400 mg, 1.25 mmol, 1.0 eq) in thionyl chloride (4.78 mL, 65.86 mmol, 52.6 eq) was stirred at 25° C. for 1 h. The resulting solution was concentrated under reduced pressure to give 5-amino-1-[2-(difluoromethyl)-3-fluoro-4-(fluoromethoxy)phenyl]imidazole-4-carbonyl chloride (400 mg, 1.18 mmol, 94% yield), which was used without further purification. MS: m/z=334.0 [(M−Cl+OCH3)+H]+, ESI pos

### Step 3: tert-butyl 4-[4-[[5-amino-1-[2-(difluoromethyl)-3-fluoro-4-(fluoromethoxy)phenyl]imidazole-4-carbonyl]amino]-2-chloro-benzoyl]piperazine-1-carboxylate

To a solution of tert-butyl 4-(4-amino-2-chloro-benzoyl)piperazine-1-carboxylate ([1338249-09-4], 402 mg, 1.18 mmol, 1.0 eq) and pyridine (1.92 mL, 23.69 mmol, 20.0 eq) in CH₂Cl₂ (1 mL) was added a suspension of 5-amino-1-[2-(difluoromethyl)-3-fluoro-4-(fluoromethoxy)phenyl]imidazole-4-carbonyl chloride (400 mg, 1.18 mmol, 1.0 eq) in CH₂Cl₂ (1 mL) and the reaction stirred at RT. Then the reaction was concentrated to dryness and the residue was taken up in CH₂Cl₂ (3 ml) and the organics washed with 2×3 ml H₂O then 1×3 ml brine solution. The organics were dried (Na₂SO₄) before concentration to dryness. The crude was then purified by flash column chromatography eluting 4% MeOH in CH₂Cl₂. The desired fractions were concentrated under reduced pressure to afford the title compound (600 mg, 0.94 mmol, 79% yield). MS: m/z=640.9 [M+H]+, ESI pos

### Step 4: 5-amino-N-[3-chloro-4-(piperazine-1-carbonyl)phenyl]-1-[2-(difluoromethyl)-3-fluoro-4-(fluoromethoxy)phenyl]imidazole-4-carboxamide

To a solution of tert-butyl 4-[4-[[5-amino-1-[2-(difluoromethyl)-3-fluoro-4-(fluoromethoxy)phenyl]imidazole-4-carbonyl]amino]-2-chloro-benzoyl]piperazine-1-carboxylate (600 mg, 0.94 mmol, 1.0 eq) and trifluoroacetic acid (1.0 mL, 12.98 mmol, 13.87 eq) in CH₂Cl₂ (3 mL) was stirred at 25° C. for 1 h. The resulting solution was concentrated under reduced pressure to give the title compound (400 mg, 0.74 mmol, 78% yield), which was used without further purification. MS: m/z=541.0 [M+H]+, ESI pos

### Intermediate 134

### 5-amino-1-[4-(cyclopropoxy)-2,3-difluoro-phenyl]imidazole-4-carboxylic acid

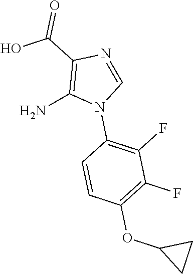 [SMILES: Nc1c(C(=O)O)ncn1-c1ccc(OC2CC2)c(F)c1F]

### Step 1: 3-chloro-1-(cyclopropoxy)-2-fluoro-4-nitro-benzene

To a solution of cyclopropanol (0.48 g, 8.27 mmol, 0.64 eq) in THF (35 mL) was added sodium hydride (0.31 g, 12.92 mmol, 1.0 eq) at 0° C. under nitrogen atmosphere. The mixture was stirred at 0° C. for 0.5 hr. Then 3-chloro-1,2-difluoro-4-nitro-benzene (2.5 g, 12.92 mmol, 1.0 eq) was added. The mixture was stirred at 25° C. for 16 h The reaction mixture was quenched by addition saturated ammonium chloride solution 40 mL at 0° C., and then extracted with ethyl acetate (6 mL×3). The combined organic layers were washed with brine 10 mL, dried over anhydrous sodium sulfate, filtered and concentrated under reduced pressure. The residue was purified by column chromatography eluting 10% EtOAc in Isohexane. The desired fractions were concentrated to dryness in vacuo to afford the title compound (2100 mg, 9.07 mmol, 70.19% yield) as a yellow oil. 1H NMR (400 MHz, DMSO) δ 8.11 (dd, J=9.3, 1.9 Hz, 1H), 7.62 (dd, J=9.2, 8.2 Hz, 1H), 4.18 (dt, J=8.9, 3.0 Hz, 1H), 0.88 (dd, J=10.8, 4.8 Hz, 2H), 0.83-0.79 (m, 2H).

### Step 2: 1-(cyclopropoxy)-2,3-difluoro-4-nitro-benzene

To a solution of 3-chloro-1-(cyclopropoxy)-2-fluoro-4-nitro-benzene (2100 mg, 9.07 mmol, 1.0 eq) in DMSO (40 mL) was added cesium fluoride (2755 mg, 18.13 mmol, 2.0 eq). The mixture was stirred at 140° C. for 5 h. The reaction mixture was quenched by addition water 30 mL at 0° C., and then extracted with ethyl acetate (40 mL×3). The combined organic layers were washed with brine 40 mL, dried over anhydrous sodium sulfate, filtered and concentrated under reduced pressure. The residue was purified by silica gel chromatography eluting 10% EtOAc in Isohexane. The desired fractions were concentrated to dryness in vacuo to afford the tile compound (1200 mg, 5.58 mmol, 49.21% yield) as a brown oil.

### Step 3: 4-(cyclopropoxy)-2,3-difluoro-aniline

A solution of 1-(cyclopropoxy)-2,3-difluoro-4-nitro-benzene (1200 mg, 5.58 mmol, 1.0 eq) in ethanol (12 mL) and water (3 mL) were added iron powder (1561 mg, 27.89 mmol, 5.0 eq), and ammonium chloride (1491 mg, 27.89 mmol, 5.0 eq) after which the reaction was stirred at 50° C. for 5 h. The suspension was filtered, and the filtrate was diluted with EA (50 mL) and washed with water (25 mL×3), dried over Na₂SO₄ and evaporated under reduced pressure to afford the title compound (720 mg, 3.89 mmol, 64.34% yield) as a brown oil. MS [M+H]+: 186.1

### Step 4: ethyl 5-amino-1-[4-(cyclopropoxy)-2,3-difluoro-phenyl]imidazole-4-carboxylate

To a solution of ethyl 2-amino-2-cyanoacetate; 4-methylbenzene-1-sulfonic acid (2335 mg, 7.78 mmol, 2.0 eq) were added triethylamine (2.17 mL, 15.55 mmol, 4.0 eq) and triethyl orthoformate (8407 mg, 56.73 mmol, 14.59 eq), the resultant mixture was stirred at 80° C. for 2 h. Then, the solvent was removed under vacuo and the residue was diluted with ACN (4 mL), then 4-(cyclopropoxy)-2,3-difluoro-aniline (720 mg, 3.89 mmol, 1.0 eq) was added and the resultant mixture was stirred at 50° C. for 16 h The reaction was concentrated to dryness and the residue was taken up in EtOAc (30 ml) and the organic layers washed with 2×30 ml water then 1×30 ml brine. The organic layers was separated and dried (MgSO4) before concentration to dryness. The crude was then purified by silica gel chromatography eluting 100% EtOAc in Isohexane. The desired fractions were concentrated to dryness in vacuo to afford the title compound (700 mg, 2.17 mmol, 55.68% yield) as a brown oil. MS [M+H]+: 324.0

### Step 5: 5-amino-1-[4-(cyclopropoxy)-2,3-difluoro-phenyl]imidazole-4-carboxylic acid

Ethyl 5-amino-1-[4-(cyclopropoxy)-2,3-difluoro-phenyl]imidazole-4-carboxylic acid (460 mg, 1.56 mmol, 56.74% yield) was dissolved in IPA (70 mL). A solution of sodium hydroxide (445 mg, 11.14 mmol, 5.0 eq) in water (35 mL) was added dropwise at 20° C. and stirred at 50° C. for 12 h. Then the reaction was concentrated. The residue was diluted with water (10 mL), and the solution was subjected to extraction with ethyl acetate (2×10⁰ mL). The aqueous layer was acidified to pH 5 with HCl (2M), and the product was collected by filtration afford the title compound (460 mg, 1.56 mmol, 69.96% yield). MS [M+H]⁺: 296.0

### Example Compounds

### Compound 1

### 5-amino-N-[4-[4-((1s,3s)-3-aminocyclobutanecarbonyl)piperazine-1-carbonyl]-3-chlorophenyl]-1-[2,3-difluoro-4-(fluoromethoxy)phenyl]imidazole-4-carboxamide 0.1:1.2 2,2,2-trifluoroacetic acid

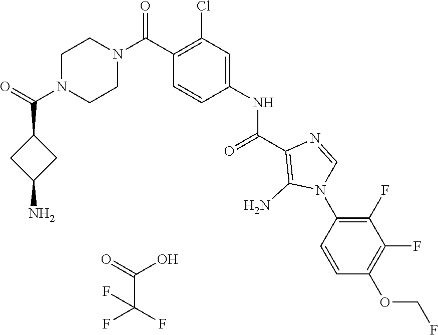 [SMILES: Nc1c(C(=O)Nc2ccc(C(=O)N3CCN(C(=O)[C@H]4C[C@@H](N)C4)CC3)c(Cl)c2)ncn1-c1ccc(OCF)c(F)c1F ; O=C(O)C(F)(F)F]

Under argon atmosphere intermediate 11 N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester (40 mg, 52.6 μmol, 1.00 eq) was dissolved in N,N-dimethylformamide (1.5 mL). Then DIEA (27.16 mg, 36.71 μL, 210.18 μmol, 4.000 eq) was added, followed by cis-3-(tert-butoxycarbonylamino)cyclobutanecarboxylic acid (12.7 mg, 57.8 μmol, 1.10 eq) and HATU (21.98 mg, 57.8 μmol, 1.100 eq). The reaction was stirred at ambient temperature overnight.

The mixture was concentrated under reduced pressure and the residue was dissolved in dichloromethane (2 mL) and treated with TFA (479.3 mg, 323.8 μL, 4.2 mmol, 80.0 eq). The resulting mixture was then stirred at ambient temperature for 6 hours. The mixture was concentrated under reduced pressure. The residue was re-dissolved in DMSO and directly purified by preparative reverse phase HPLC (column: Phenomenex Gemini-NX 5u 110A, I:100 mm, dia 30 mm) using water containing 0.05% TFA/acetonitrile as eluent. The corresponding fractions were and lyophilized to afford the title compound (22 mg, 55% yield). MS: 604.2 [M−H]⁻, ESI neg.

The following compounds were prepared in analogy to the procedure above

| Compound No | Reagents Used | MS |
|---|---|---|
| 2 | (2S,4S)-1-tert-butoxycarbonyl- | m/z = 680.2 |
| | 4-methoxy-proline | [M + HCOO]−, |
| | | ESI neg |
| 3 | (3R,6S)-1-tert-butoxycarbonyl- | m/z = 678.7 |
| | 6-methyl-nipecotic acid | [M + HCOO]−, |
| | | ESI neg |
| 4 | (2R,5S)-4-tert-butoxycarbonyl- | m/z = 680.6 |
| | 5-methyl-morpholine-2- | [M + HCOO]−, |
| | carboxylic acid | ESI neg |
| 5 | tetrahydropyran-4-carboxylic | m/z = 621.3 |
| | acid | [M + H]+ |
| 6 | 1-azabicyclo[2.2.1]heptane- | m/z = 676.2 |
| | 4-carboxylic acid; hydrochloride | [M + HCOO]−, |
| | | ESI neg |
| 7 | (3R,5S)-1-tert-butoxycarbonyl- | m/z = 664.2 |
| | 5-methyl-pyrrolidine-3- | [M + HCOO]−, |
| | carboxylic acid | ESI neg |
| 8 | (3S,6R)-4-tert-butoxycarbonyl- | m/z = 680.3 |
| | 6-methyl-morpholine-3- | [M + HCOO]−, |
| | carboxylic acid | ESI neg |
| 9 | (2S,3R)-1-tert-butoxycarbonyl- | m/z = 604.3 |
| | 3-methyl-azetidine-2- | [M − H]−, |
| | carboxylic acid | ESI neg |
| 10 | (2S,4R)-1-tert-butoxycarbonyl- | 678.5 |
| | 4-methyl-pipecolinic acid | [M + HCOO]−, |
| | | ESI neg |
| 11 | cis-4-(tert- | m/z = 678.6 |
| | butoxycarbonylamino)cyclo- | [M + HCOO]−, |
| | hexanecarboxylic acid | ESI neg |
| 12 | 2-keto-1-methyl-isonipecotic | m/z = 648.3 |
| | acid, water + 0.05% HCl as | [M + H]+ |
| | eluent for final purification | |
| 13 | (2S,5S)-1-tert-butoxycarbonyl- | m/z = 664.2 |
| | 5-methyl-proline | [M + HCOO]−, |
| | | ESI neg |
| 14 | (1R,3S)-3-(tert- | m/z = 664.3 |
| | butoxycarbonylamino)cyclo- | [M + HCOO]−, |
| | pentanecarboxylic acid | ESI neg |
| 15 | trans-3-[(tert- | m/z = 664.6 |
| | butoxycarbonylamino)methyl]cyclo- | [M + HCOO]−, |
| | butanecarboxylic acid | ESI neg |
| 16 | (2S,4S)-1-tert-butoxycarbonyl- | m/z = 650.5 |
| | 4-methyl-azetidine-2- | [M + HCOO]−, |
| | carboxylic acid | ESI neg |
| 17 | (2S,4S)-1-tert-butoxycarbonyl- | m/z = 679.4 |
| | 4-[(tert-butoxycarbonyl- | [M + HCOO]−, |
| | amino)methyl]proline | ESI neg |
| 18 | Methylamine hydrochloride | m/z = 454.3 |
| | | [M + H]+, |
| | | ESI pos |
| 19 | (2S)-1-tert-butoxycarbonyl- | m/z = 664.6 |
| | 3,3-dimethyl-azetidine-2- | [M + HCOO]−, |
| | carboxylic acid | ESI neg |
| 20 | 1-tert-butoxycarbonyl-4-hydroxy- | 680.6 |
| | isonipecotic acid | [M + HCOO]−, |
| | | ESI neg |
| 21 | 1-tert-butoxycarbonyl-4- | m/z = 714.5 |
| | (difluoromethyl)isonipecotic acid | [M + HCOO]−, |
| | | ESI neg |
| 22 | (2S,5R)-1-tert-butoxycarbonyl- | m/z = 678.4 |
| | 5-methyl-pipecolinic acid | [M + HCOO]−, |
| | | ESI neg |
| 23 | cis-4-(tert-butoxycarbonylamino)- | m/z = 696.4 |
| | 1-fluoro-cyclohexanecarboxylic acid | [M + HCOO]−, |
| | | ESI neg |
| 24 | boc-L-alanine | m/z = 578.4 |
| | | [M − H+]−, |
| | | ESI neg |
| 25 | 2-tert-butoxycarbonyl-2- | m/z = 632.2 |
| | azaspiro[3.3]heptane-6- | [M + H]+, |
| | carboxylic acid | ESI pos |
| 26 | (1R,5S,6r)-3-ethyl-3- | m/z = 690.2 |
| | azabicyclo[3.1.0]hexane-6- | [M + HCOO]−, |
| | carboxylic acid; hydrochloride | ESI neg |
| | (intermediate 29) | |
| 27 | (2S,4R)-1-tert-butoxycarbonyl- | m/z = 618.2 |
| | 4-methyl-proline | [M − H+]−, |
| | | ESI neg |
| 28 | trans-3-(tert- | m/z = 650.4 |
| | butoxycarbonylamino)cyclo- | [M + HCOO]−, |
| | butanecarboxylic acid | ESI neg |
| 29 | (1R,5S,6r)-3-methyl-3- | m/z = 676.2 |
| | azabicyclo[3.1.0]hexane- | [M + HCOO]−, |
| | 6-carboxylic acid hydrochloride | ESI neg |
| 30 | (2S,4S)-1-tert-butoxycarbonyl- | m/z = 664.2 |
| | 4-methyl-proline | [M + HCOO]−, |
| | | ESI neg |
| 31 | 1-methylisonipecotic acid, | m/z = 632.3 |
| | extraction after amide coupling, | [M − H]−, |
| | final purification with acetonitrile/ | ESI neg |
| | water + 0.05% HCl | |
| 32 | (3S)-1-tert-butoxycarbonyl-3- | m/z = 682.4 |
| | fluoro-nipecotic acid | [M + HCOO]−, |
| | | ESI neg |
| 33 | (1S,5R,6r)-3-tert-butoxycarbonyl- | m/z = 680.6 |
| | 6-fluoro-3-azabicyclo[3.1.0]hexane- | [M + HCOO]−, |
| | 6-carboxylic acid | ESI neg |
| 34 | 1-tert-butoxycarbonyl-4-fluoro- | m/z = 682.5 |
| | isonipecotic acid | [M + HCOO]−, |
| | | ESI neg |
| 35 | (3S)-4-tert- | m/z = 666.4 |
| | butoxycarbonylmorpholine- | [M + HCOO]−, |
| | 3-carboxylic acid | ESI neg |
| 36 | (2R)-4-tert- | m/z = 666.4 |
| | butoxycarbonylmorpholine- | [M + HCOO]−, |
| | 2-carboxylic acid | ESI neg |
| 37 | (3R)-1-tert- | m/z = 664.3 |
| | butoxycarbonylnipecotic acid | [M + HCOO]−, |
| | | ESI neg |
| 38 | (2S)-1-tert- | m/z = 664.5 |
| | butoxycarbonylpipecolinic acid | [M + HCOO]−, |
| | | ESI neg |
| 39 | 1-tert-butoxycarbonylazetidine- | m/z = 590.3 |
| | 3-carboxylic acid | [M − H]−, |
| | | ESI neg |
| 40 | (2S)-1-tert- | m/z = 590.3 |
| | butoxycarbonylazetidine- | [M − H]−, |
| | 2-carboxylic acid | ESI neg |
| 41 | (2S,4S)-1-tert-butoxycarbonyl- | m/z = 682.4 |
| | 4-(fluoromethyl)proline | [M + HCOO]−, |
| | | ESI neg |
| 42 | (3S)-1-tert- | m/z = 606.3 |
| | butoxycarbonylpyrrolidine- | [M + H]+, |
| | 3-carboxylic acid | ESI pos |
| 43 | (2S,4R)-1-tert-butoxycarbonyl- | m/z = 680.2 |
| | 4-methylol-proline | [M + HCOO]−, |
| | | ESI neg |
| 44 | (2S,3S)-1-tert-butoxycarbonyl- | m/z = 666.2 |
| | 3-hydroxy-proline | [M + HCOO]−, |
| | | ESI neg |
| 45 | (2S,4S)-1-tert-butoxycarbonyl- | m/z = 675.2 |
| | 4-cyano-proline | [M + HCOO]−, |
| | | ESI neg |
| 46 | (2S,4S)-1-tert-butoxycarbonyl- | m/z = 666.2 |
| | 4-hydroxy-proline | [M + HCOO]−, |
| | | ESI neg |
| 47 | (6S)-5-tert-butoxycarbonyl- | m/z = 712.2 |
| | 2,2-difluoro-5- | [M + HCOO]−, |
| | azaspiro[2.4]heptane- | ESI neg |
| | 6-carboxylic acid | |
| | [1357482-03-1] | |
| 48 | (1S,3S,5S)-2-tert-butoxycarbonyl- | m/z = 662.2 |
| | 2-azabicyclo[3.1.0]hexane- | [M + HCOO]−, |
| | 3-carboxylic acid | ESI neg |
| 49 | Boc-D-Pro-OH | m/z = 650.2 |
| | | [M + HCOO]−, |
| | | ESI neg |
| 50 | (3R)-1-tert- | m/z = 650.2 |
| | butoxycarbonylpyrrolidine- | [M + HCOO]−, |
| | 3-carboxylic acid | ESI neg |
| 51 | 1,3,3a,4,5,6,7,7a- | m/z = 705.2[M + |
| | octahydropyrrolo[3,4- | HCOO]−, |
| | c]pyridine-2-carboxylic | ESI neg |
| | acid tert-butyl ester | |
| 52 | Boc-L-proline | m/z = 650.2 |
| | | [M + HCOO]−, |
| | | ESI neg |
| 53 | 5-amino-N-[3-chloro-4-(piperazine-1- | m/z = 635.3 |
| | carbonyl)phenyl]-1-[2,3-difluoro- | [M + H]+ |
| | 4-(fluoromethoxy)phenyl]imidazole- | |
| | 4-carboxamide; dihydrochloride | |
| 54 | cis-2-Boc-3,3a,4,5,6,6a-hexahydro- | m/z = 646.3 |
| | 1H-cyclopenta[c]pyrrole- | [M + H]+ |
| | 5-carboxylic acid | |
| 55 | 1,1-diketothiane-4-carboxylic acid | m/z = 669.3 |
| | | [M + H]+ |
| 56 | (1R,5S,6r)-3-tert-butoxycarbonyl- | m/z = 618.18 |
| | 3-azabicyclo[3.1.0]dhexane-6- | [M + H]+, |
| | carboxylic acid | ESI pos |
| 57 | (2S,3aS,6aS)-1-tert-butoxycarbonyl- | m/z = 779.4 |
| | 5-carbobenzoxy-2,3,3a,4,6,6a- | [M − H]−, |
| | hexahydropyrrolo[3,4-b]pyrrole- | ESI neg |
| | 2-carboxylic acid | |

The following compounds were prepared in analogy to the procedure above, starting from N-[5-[[3-chloro-4-(piperazine-1-carbonyl)phenyl]carbamoyl]-3-[2-(difluoromethyl)-3-fluoro-4-(fluoromethoxy)phenyl]imidazol-4-yl]carbamic acid tert-butyl ester (intermediate 14)

| Compound No | Reagents Used | MS |
|---|---|---|
| 58 | 1-tert-butoxycarbonyl-4-fluoro- | m/z = 714.5 |
| | isonipecotic acid, purification | [M + HCOO]−, |
| | of the intermediate by prep HPLC | ESI neg |
| 59 | (1R,5S,6r)-3-methyl-3- | m/z = 708.2 |
| | azabicyclo[3.1.0]hexane- | [M + HCOO]−, |
| | 6-carboxylic acid hydrochloride | ESI neg |
| 60 | 1-methylisonipecotic acid | m/z = 664.2 |
| | | [M − H]−, |
| | | ESI neg |

The following compounds were prepared in analogy to the procedure above, starting from N-[3-[2,3-difluoro-4-(fluoromethoxy)phenyl]-5-[[3-fluoro-4-(piperazine-1-carbonyl)phenyl]carbamoyl]imidazol-4-yl]carbamic acid tert-butyl ester;2,2,2-trifluoroacetic acid (intermediate 12)

| Compound No | Reagents Used | MS |
|---|---|---|
| 61 | 1-tert-butoxycarbonyl-3-fluoro- | m/z = 666.4 |
| | nipecotic acid, purification of | [M + HCOO]−, |
| | the intermediate by prep HPLC | ESI neg |
| 62 | 1-tert-butoxycarbonyl-4-fluoro- | m/z = 666.6 |
| | isonipecotic acid | [M + HCOO]−, |
| | | ESI neg |
| 63 | (2S,4S)-1-tert-butoxycarbonyl- | m/z = 648.3 |
| | 4-methyl-proline | [M + HCOO]−, |
| | | ESI neg |
| 64 | (1R,5S,6r)-3-methyl-3- | m/z = 660.3 |
| | azabicyclo[3.1.0]hexane- | [M + HCOO]−, |
| | 6-carboxylic acid hydrochloride | ESI neg |

The following compounds were prepared in analogy to the procedure above, starting from N-[3-[2,3-difluoro-4-(fluoromethoxy)phenyl]-5-[[4-(piperazine-1-carbonyl)phenyl]carbamoyl]imidazol-4-yl]carbamic acid tert-butyl ester (intermediate 13)

| Compound No | Reagents Used | MS |
|---|---|---|
| 65 | (1R,5S,6r)-3-methyl-3- | m/z = 642.3 |
| | azabicyclo[3.1.0]hexane- | [M + HCOO]−, |
| | 6-carboxylic acid hydrochloride | ESI neg |

The following compounds were prepared in analogy to the procedure above, starting from N-[5-[[3-chloro-4-(piperazine-1-carbonyl)phenyl]carbamoyl]-3-[4-(difluoromethoxy)-2,3-difluorophenyl]imidazol-4-yl]carbamic acid tert-butyl ester (intermediate 15)

| Compound No | Reagents Used | MS |
|---|---|---|
| 66 | 1-methylisonipecotic acid | m/z = 696.3 |
| | | [M + HCOO]−, |
| | | ESI neg |
| 67 | (1R,5S,6r)-3-methyl-3- | m/z = 694.2 |
| | azabicyclo[3.1.0]hexane- | [M + HCOO]−, |
| | 6-carboxylic acid hydrochloride | ESI neg |

The following compounds were prepared in analogy to the procedure above, starting from 4-[[5-(tert-butoxycarbonylamino)-1-[2,3-difluoro-4-(fluoromethoxy)phenyl]imidazole-4-carbonyl]amino]-2-chlorobenzoic acid (intermediate 10)

| Cmpd No | Reagents Used | MS |
|---|---|---|
| 68 | rac-tert-butyl (3R,4R)-3-hydroxy- | m/z = 681.4 |
| | 4-(piperazine-1-carboxamido)pyrrolidine- | [M + HCOO]−, |
| | 1-carboxylate (intermediate 31) | ESI neg |
| 69 | methyl exo-3-azabicyclo[3.1.0]hexane- | m/z = 564.3 |
| | 6-carboxylate; hydrochloride | [M + H]+, |
| | [1212063-26-7], purification | ESI pos |
| | of the intermediate by prep-HPLC | |

### Compound 70

### 5-amino-N-[4-[[1-[(1R,5S,6r)-3-azabicyclo[3.1.0]hexane-6-carbonyl]azetidin-3-yl]carbamoyl]-3-chloro-phenyl]-1-[2,3-difluoro-4-(fluoromethoxy)phenyl]imidazole-4-carboxamide 0.1:1.3 2,2,2-trifluoroacetic acid

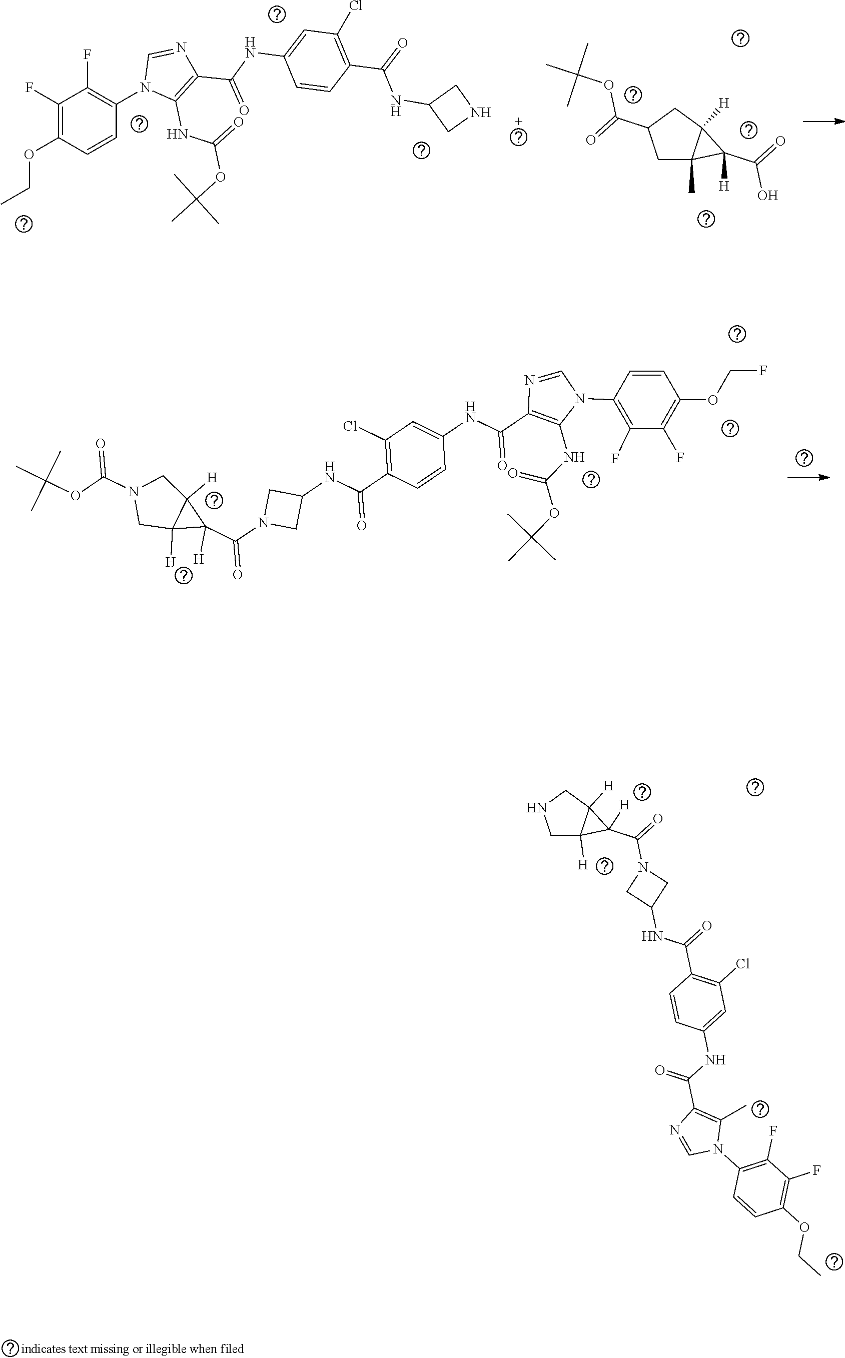 [SMILES: CCOc1ccc(-n2cnc(C(=O)Nc3ccc(C(=O)NC4CNC4)c(Cl)c3)c2NC(=O)OC(C)(C)C)c(F)c1F ; CC(C)(C)OC(=O)C1C[C@H]2[C@H](C(=O)O)[C@]2(C)C1 ; CC(C)(C)OC(=O)Nc1c(C(=O)Nc2ccc(C(=O)NC3CN(C(=O)C4C5CN(C(=O)OC(C)(C)C)CC54)C3)c(Cl)c2)ncn1-c1ccc(OCF)c(F)c1F ; CCOc1ccc(-n2cnc(C(=O)Nc3ccc(C(=O)NC4CN(C(=O)C5C6CNCC65)C4)c(Cl)c3)c2C)c(F)c1F]

Under argon atmosphere N-[5-[[4-(azetidin-3-ylcarbamoyl)-3-chloro-phenyl]carbamoyl]-3-[2,3-difluoro-4-(fluoromethoxy)phenyl]imidazol-4-yl]carbamic acid tert-butyl ester (intermediate 17, 52 mg, 78.7 μmol, 1.00 eq) was dissolved in N,N-dimethylformamide (2 mL), then DIEA (40.66 mg, 54.95 μL, 314.6 μmol, 4.000 eq) was added, followed by (1R,5S,6r)-3-tert-butoxycarbonyl-3-azabicyclo[3.1.0]hexane-6-carboxylic acid (19.66 mg, 86.53 μmol, 1.100 eq) and HATU (32.9 mg, 86.5 μmol, 1.10 eq). The reaction was stirred at ambient temperature for 2 hours. The mixture was concentrated under reduced pressure. The residue was purified by preparative reverse phase HPLC (column: Phenomenex Gemini-NX 5u 110A, I:100 mm, dia 30 mm) using water containing 0.05% TFA/acetonitrile as eluent. The corresponding fractions were collected and freeze dried from water/acetonitrile. Mixed fractions were again purified by preparative reverse phase HPLC under the same conditions. Combined purified fractions afforded 22 mg intermediate.

The material was dissolved in dichloromethane (2 mL) and TFA (448.44 mg, 303 μL, 3.93 mmol, 50.0 eq) was added. The mixture was then stirred at ambient temperature for 17 h. Then the solvent was evaporated and the residue purified by preparative reverse phase HPLC (column: Phenomenex Gemini-NX 5u 110A, 1:100 mm, dia 30 mm) using water containing 0.05% TFA/acetonitrile as eluent. The corresponding fractions were were collected and freeze dried afford the title compound (10 mg, 18% yield). MS: 604.17 [M+H]⁺, ESI pos, ratio parent: CF₃COOH by 19F NMR=1:1.3.

### Compound 71

### 5-amino-N-[4-[4-[3-(aminomethyl) azetidine-1-carbonyl]piperazine-1-carbonyl]-3-chlorophenyl]-1-[2,3-difluoro-4-(fluoromethoxy)phenyl]imidazole-4-carboxamide 0.1:1.2 2,2,2-trifluoroacetic acid

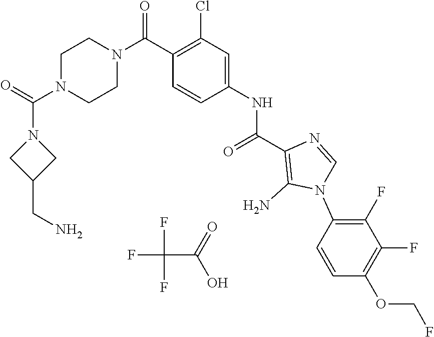 [SMILES: NCC1CN(C(=O)N2CCN(C(=O)c3ccc(NC(=O)c4ncn(-c5ccc(OCF)c(F)c5F)c4N)cc3Cl)CC2)C1 ; O=C(O)C(F)(F)F]

To a solution of N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester (intermediate 11, 50 mg, 82.1 μmol, 1.00 eq) in dichloromethane (2 mL) was added DIEA (63.67 mg, 86.03 μL, 492.6 μmol, 6.000 eq). The reaction was cooled to 0° C. and triphosgene (9.75 mg, 32.8 μmol, 0.400 eq) was added. After 20 min N-(azetidin-3-ylmethyl) carbamic acid tert-butyl ester (30.58 mg, 164.2 μmol, 2.000 eq) was added and the reaction was stored 21 hours in the refrigerator. A few drops of water were added and the mixture was concentrated under reduced pressure. The residue was dissolved in DMSO and the resulting solution was directly purified by preparative reverse phase HPLC (column: Phenomenex Gemini-NX 5u 110A, I:100 mm, dia 30 mm) using water containing 0.05% TFA/acetonitrile as eluent. The corresponding fractions were collected and lyophilized to afford (Intermediate MS: 819.6 [M−H]⁻, ESI neg).

The residue was dissolved in dichloromethane (2 mL) and TFA (748.9 mg, 506.0 μL, 6.57 mmol, 80.00 eq) was added. The clear solution was stirred at room temperature for 21 hours. Then again TFA (748.9 mg, 506.0 μL, 6.57 mmol, 80.0 eq) was added and stirring continued for 2 hours. The mixture was concentrated under reduced pressure. The residue was dissolved in DMSO and the solution was directly purified by preparative reverse phase HPLC (column: Phenomenex Gemini-NX 5u 110A, I:100 mm, dia 30 mm) using water containing 0.05% TFA/acetonitrile as eluent. The corresponding were combined and lyophilized to afford the title compound (11 mg; 17% yield). MS: 665.4 [M+HCOO]⁻, ESI neg.

The following compounds were prepared in analogy to the procedure above

| Compound No | Reagents Used | MS |
|---|---|---|
| 72 | 5-methyl-2-oxa-5,8- | 721.4 |
| | diazaspiro[3.5]nonane; | [M + HCOO]−, |
| | hydrochloride warmed to ambient | ESI neg |
| | temperature during urea formation | |
| 73 | 2-methyl-2,8- | 719.4 |
| | diazaspiro[3.5]nonane; | [M + HCOO]−, |
| | dihydrochloride | ESI neg |

### Compound 74

### 5-amino-N-[3-chloro-4-[4-[(1S,4S)-5-methyl-2,5-diazabicyclo[2.2.1]heptane-2-carbonyl]piperazine-1-carbonyl]phenyl]-1-[2,3-difluoro-4-(fluoromethoxy)phenyl]imidazole-4-carboxamide. 1:2 2,2,2-trifluoroacetic acid

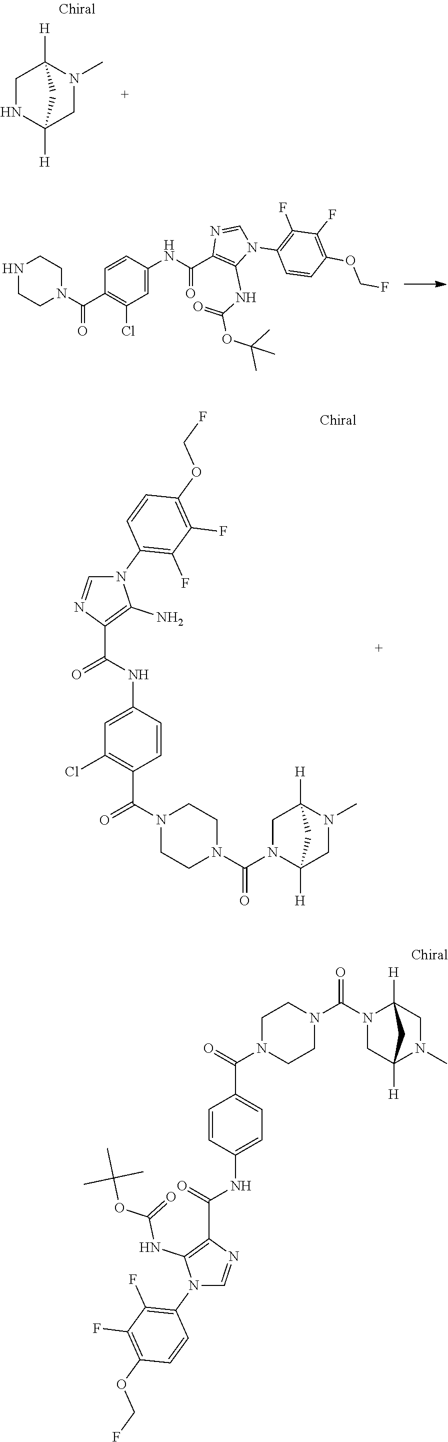

To a clear solution of N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester;2,2,2-trifluoroacetic acid (intermediate 11, 30 mg, 41.1 μmol, 1.00 eq) in dichloromethane (1 mL) was added DIEA (23.89 mg, 32.28 μL, 184.8 μmol, 4.500 eq) followed by triphosgene (4.88 mg, 16.4 μmol, 0.400 eq) at 0° C. After 20 min a solution of (1S,4S)-2-methyl-2,5-diazabicyclo[2.2.1]heptane dihydrobromide (22.51 mg, 82.16 μmol, 2.000 eq) and DIEA (10.62 mg, 14.35 μL, 82.16 μmol, 2.000 eq) in dichloromethane (1 mL) was added to the white suspension and the mixture was stirred in the ice-bath for further 20 min. The reaction was diluted with water and extracted with dichloromethane. The combined organic layers were washed with brine, dried over magnesium sulfate, filtered and concentrated under reduced pressure. The residue was purified using silica gel chromatography using dichloromethane/(methanol+0.25% NH₄OH) as eluent to afford 13 mg as intermediate. m/z=745.7 [M−H]⁻, ESI neg.

The residue was dissolved in dichloromethane (1 mL) and TFA (234.18 mg, 158.23 μL, 2.05 mmol, 50.0 eq) was added at 0° C. The mixture was allowed to warm up to ambient temperature after 10 min and the reaction stirred 20 hours at room temperature. The mixture was concentrated under reduced pressure and purified by reverse phase chromatography over a Phenomenex Gemini-NX 5u 110A, I:100 mm, dia: 30 mm column using acetonitrile/water (+0.05% TFA) as eluent. The corresponding fractions were lyophilized to afford the title compound (7.3 mg, 20% yield), m/z=691.7 [M−H]⁻, ESI neg.

The following compounds were prepared in analogy to the procedure above

| Compound No | Reagents | MS |
|---|---|---|
| 75 | 1-methylpiperazin-2-one | m/z = 647.3 |
| | | [M − H]− |
| 76 | (1S,4S)-2,5- | m/z = 677.2 |
| | diazabicyclo[2.2.1]heptane- | [M + HCOO]−, |
| | 2-carboxylic acid tert-butyl ester | ESI neg |
| 77 | cis-2-Boc- | m/z = 691.4 |
| | hexahydropyrrolo[3,4- | [M + HCOO]−, |
| | c]pyrrole | ESI neg |
| 78 | (1R,4R)-2,5- | m/z = 677.2 |
| | diazabicyclo[2.2.1]heptane- | [M + HCOO]−, |
| | 2-carboxylic acid tert-butyl ester | ESI neg |
| 79 | 1,4-thiazinane 1,1-dioxide | m/z = 670.2[M + |
| | | H]+, ESI pos |
| 80 | 1-methylpiperazine | m/z = 635.3 |
| | | [M + H]+, |
| | | ESI pos |
| 81 | 2,6-diazaspiro[3.3]heptane- | m/z = 631.3 |
| | 2-carboxylic acid tert- | [M − H]−, |
| | butyl ester | ESI neg |

The following compounds were prepared in analogy to the procedure above starting from N-[5-[[3-chloro-4-(piperazine-1-carbonyl)phenyl]carbamoyl]-3-[2-fluoro-4-(fluoromethoxy)phenyl]imidazol-4-yl]carbamic acid tert-butyl ester (intermediate 16)

| Compound No | Reagents | MS |
|---|---|---|
| 82 | 1-Boc-piperazine | m/z = 601.3 |
| | | [M − H]−, |
| | | ESI neg |

### Compound 83

### 5-amino-N-[3-chloro-4-[4-(piperazine-1-carbonyl)piperazine-1-carbonyl]phenyl]-1-[2,3-difluoro-4-(fluoromethoxy)phenyl]imidazole-4-carboxamide. 1:1.2 2,2,2-trifluoroacetic acid

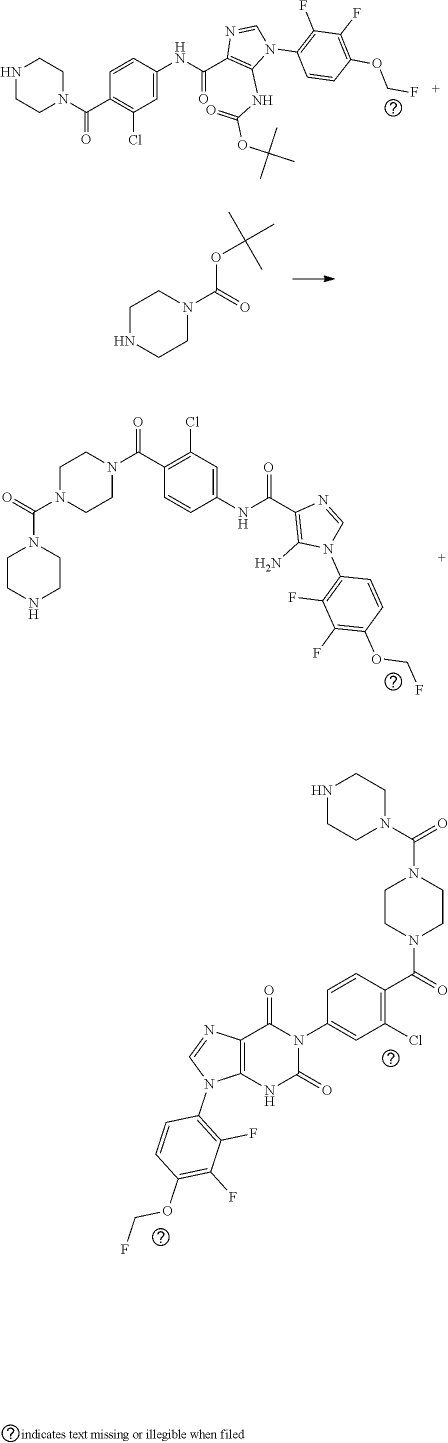 [SMILES: CC(C)(C)OC(=O)Nc1c(C(=O)Nc2ccc(C(=O)N3CCNCC3)c(Cl)c2)ncn1-c1ccc(OCF)c(F)c1F ; CC(C)(C)OC(=O)N1CCNCC1 ; Nc1c(C(=O)Nc2ccc(C(=O)N3CCN(C(=O)N4CCNCC4)CC3)c(Cl)c2)ncn1-c1ccc(OCF)c(F)c1F ; O=C(c1ccc(-n2c(=O)[nH]c3c(ncn3-c3ccc(OCF)c(F)c3F)c2=O)cc1Cl)N1CCN(C(=O)N2CCNCC2)CC1]

To a clear solution of N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester;2,2,2-trifluoroacetic acid (intermediate 11, 50 mg, 69.15 μmol, 1.000 eq) and DIEA (40.22 mg, 54.35 μL, 311.19 μmol, 4.500 eq) in dichloromethane (3.5 mL) was added triphosgene (8.21 mg, 27.7 μmol, 0.400 eq). After 30 min 1-Boc-piperazine (25.76 mg, 138.31 μmol, 2.000 eq) was added and the mixture stirred 15 hours. The reaction was diluted with water and extracted 3 times with dichloromethane. The combined organic layers were combined, washed with brine, dried over magnesium sulfate, filtered and evaporated. The residue was dissolved in dichloromethane (2 mL) and TFA (473.1 mg, 319.7 μL, 4.15 mmol, 60.00 eq) was added and the mixture stirred 6 hours at room temperature, then stored in the refrigerator overnight. The solvent was evaporated. The residue was directly purified by preparative reverse phase HPLC (column: Phenomenex Gemini-NX 5u 110A, I:100 mm, dia 30 mm) using water containing 0.05% TFA/acetonitrile as eluent. The corresponding fractions were combined and lyophilized to afford the title compound (26.4 mg, 50% yield). m/z=665.4 [M+HCOO]⁻, ESI neg.

### Compound 84

### 5-amino-N-[3-chloro-4-[4-(piperazine-1-carbonyl)piperidine-1-carbonyl]phenyl]-1-[2,3-difluoro-4-(fluoromethoxy)phenyl]imidazole-4-carboxamide. 1:2 2,2,2-trifluoroacetic acid

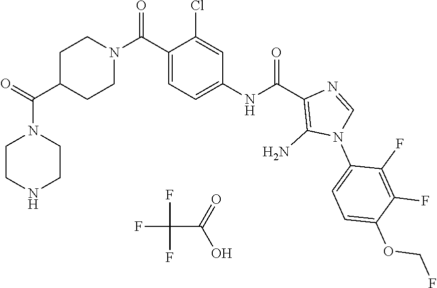 [SMILES: Nc1c(C(=O)Nc2ccc(C(=O)N3CCC(C(=O)N4CCNCC4)CC3)c(Cl)c2)ncn1-c1ccc(OCF)c(F)c1F ; O=C(O)C(F)(F)F]

To a solution of 5-(tert-butoxycarbonylamino)-1-[2,3-difluoro-4-(fluoromethoxy)phenyl]imidazole-4-carboxylic acid (intermediate 5, 32 mg, 82 μmol, 1.0 eq) and 4-[1-(4-amino-2-chloro-benzoyl) isonipecotoyl]piperazine-1-carboxylic acid tert-butyl ester (intermediate 18, 40.57 mg, 89.97 μmol, 1.100 eq) in dichloromethane (1 mL) was added 1-methylimidazole (20.15 mg, 19.56 μL, 245.4 μmol, 3.000 eq). The reaction mixture was cooled to 0° C. and n-propylphosphonic acid anhydride, cyclic trimer 50% in EtOAc (104.1 mg, 96.39 μL, 163.6 μmol, 2.000 eq) was added and stirred for 1 h at 0° C. The light solution was heated to 40° C. for 1 week. The reaction was cooled down to room temperature, concentrated under reduced pressure and the residue was purified by silica gel chromatography using dichloromethane/MeOH/NH₄OH as eluents. Corresponding fractions combined and concentrated under reduced pressure. The resulting residue was dissolved in dichloromethane (1 mL) and TFA (234.18 mg, 158.23 μL, 2.05 mmol, 50.000 eq) was added. The mixture was then stirred at ambient temperature for 20 h. The mixture was concentrated under reduced pressure and the residue was purified preparative reverse phase HPLC (column: Phenomenex Gemini-NX 5u 110A, I:100 mm, dia: 30 mm) using acetonitrile/water (+0.05% HCl) as eluent. The corresponding fractions were lyophilized to afford the title compound (20 mg, 48% yield). MS: m/z=696.4 [M+HCOO]⁻, ESI neg.

The following compounds were prepared in analogy to the procedure above

| Compound No | Reagents | MS |
|---|---|---|
| 85 | (1S,4S)-5-[1-(4-amino-2-chloro- | m/z = 676.6 |
| | benzoyl)isonipecotoyl]-2,5- | [M + HCOO]−, |
| | diazabicyclo[2.2.1]heptane-2- | ESI neg |
| | carboxylic acid tert-butyl ester | |
| | (intermediate 19) | |
| 86 | tert-butyl cis 2-[1-(4-amino-2- | m/z = 646.2 |
| | chlorobenzoyl)piperidine-4-carbonyl]- | [M + H]+ |
| | hexahydropyrrolo[3,4-c]pyrrole- | |
| | 5-carboxylate (Intermediate 20), no | |
| | purification after first step | |

### Compound 87

### 5-amino-N-[3-chloro-4-[4-(morpholine-4-carbonyl)piperazine-1-carbonyl]phenyl]-1-[2,3-difluoro-4-(fluoromethoxy)phenyl]imidazole-4-carboxamide. 1:1 2,2,2-trifluoroacetic acid

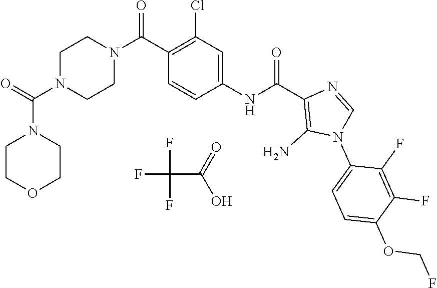 [SMILES: Nc1c(C(=O)Nc2ccc(C(=O)N3CCN(C(=O)N4CCOCC4)CC3)c(Cl)c2)ncn1-c1ccc(OCF)c(F)c1F ; O=C(O)C(F)(F)F]

To a solution of 5-(tert-butoxycarbonylamino)-1-[2,3-difluoro-4-(fluoromethoxy)phenyl]imidazole-4-carboxylic acid (intermediate 5, 24 mg, 59 μmol, 1.0 eq) in acetonitrile (700 μL) was added 1-methylimidazole (23 μL, 290 μmol, 5.0 eq). Then N,N,N′,N′-tetramethylchloroformamidinium hexafluorophosphate (TCFH)(25 mg, 88 μmol, 1.5 eq) was added. After 10 min a solution of (4-amino-2-chlorophenyl)-[4-(morpholine-4-carbonyl) piperazino]methanone (intermediate 27-A, 25 mg, 65 μmol, 1.1 eq) in acetonitrile (700 μL) was added and the mixture was stirred overnight at ambient temperature. Again TCFH (24.78 mg, 88.3 μmol, 1.500 eq) and 1-methylimidazole (23 μL, 290 μmol, 5.0 eq) were added and stirring continued for 1 hour. The mixture was concentrated under reduced pressure and the residue was purified by silica gel chromatography using first heptane/ethyl acetate, then dichloromethane/methanol+0.25% ammonium hydroxide as eluents. The corresponding fractions were concentrated under reduced pressure to afford 30 mg intermediate.

The residue was dissolved in dichloromethane (1 mL) and TFA (227 μL, 2.94 mmol, 50.0 eq) was added at ambient temperature and stirred for 2 hours. Again TFA (227 μL, 2.94 mmol, 50.0 eq) was added and the mixture was stirred overnight at ambient temperature. The mixture was concentrated under reduced pressure and the residue was purified by prep HPLC chromatography (column Phenomenex Gemini-NX 5u 110A, I:100 mm, dia: 30 mm/100×30 mm, gradient acetonitrile/water (+0.05% TFA)=10:90 to 50:50). The corresponding fractions were combined and lyophilized to afford the title compound compound (5.5 mg, 13% yield). MS: m/z=622.3 [M+H]⁺.

### Compound 88

### 5-amino-N-[3-chloro-4-(4-isonipecotoylpiperazine-1-carbonyl)phenyl]-1-[2,3-difluoro-4-(fluoromethoxy)phenyl]imidazole-4-carboxamide

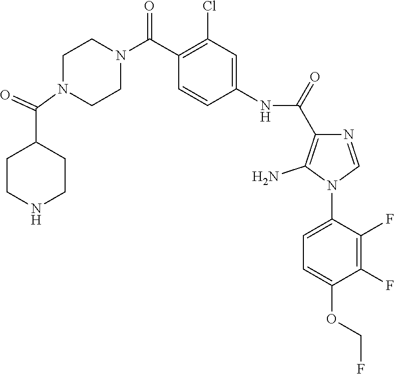 [SMILES: Nc1c(C(=O)Nc2ccc(C(=O)N3CCN(C(=O)C4CCNCC4)CC3)c(Cl)c2)ncn1-c1ccc(OCF)c(F)c1F]

### Step 1:

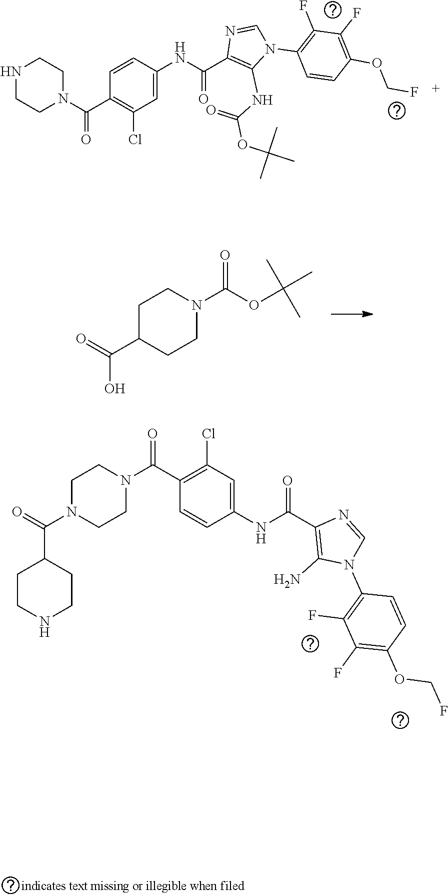 [SMILES: CC(C)(C)OC(=O)Nc1c(C(=O)Nc2ccc(C(=O)N3CCNCC3)c(Cl)c2)ncn1-c1ccc(OCF)c(F)c1F ; CC(C)(C)OC(=O)N1CCC(C(=O)O)CC1 ; Nc1c(C(=O)Nc2ccc(C(=O)N3CCN(C(=O)C4CCNCC4)CC3)c(Cl)c2)ncn1-c1ccc(OCF)c(F)c1F]

To a solution of 1-tert-butoxycarbonylisonipecotic acid (14 mg, 61 μmol, 1.2 eq) and DIEA (27 μL, 150 μmol, 3.0 eq) in N,N-dimethylformamide (500 μL) was added HATU (23 mg, 61 μmol, 1.2 eq). After 15 min a solution of N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester (intermediate 11, 31 mg, 0.051 mmol, 1.0 eq) in N,N-dimethylformamide (0.800 mL) was added and the reaction stirred for 2 hours at room temperature. The solvent was evaporated. The residue was dissolved in dichloromethane (1 mL) and TFA (196 μL, 2.55 mmol, 50.0 eq) was added and the reaction stirred for 15 hours at room temperature. The solvent was evaporated. The residue was dissolved in DMSO. This solution was directly purified by preparative reverse phase HPLC (column: Phenomenex Gemini-NX 5u 110A, I:100 mm, dia 30 mm) using water containing 0.05% TFA/acetonitrile as eluent. The corresponding fractions were collected and freeze dried. StratoSpheres SPE PLC-HCO3 MP Resin in Cartridge/200 mg-6 ml was conditioned with 3×5 ml MeOH. A solution of the above material in 10 ml CH₃CN/water 3:7 was added and allowed to run through by gravity. Resin was then washed with 4×10 ml CH₃CN/water 3:7 and 2×5 ml MeOH. The combined liquids were evaporated to remove methanol and then lyophilized to afford the title compound (10 mg, 30% yield). MS: 618.3 [M−H]⁻, ESI neg.

### Compound 89

### N-[4-[4-(1-acetimidoyl-4-fluoro-isonipecotoyl)piperazine-1-carbonyl]-3-chlorophenyl]-5-amino-1-[2,3-difluoro-4-(fluoromethoxy)phenyl]imidazole-4-carboxamide. 1:2 2,2,2-trifluoroacetic acid

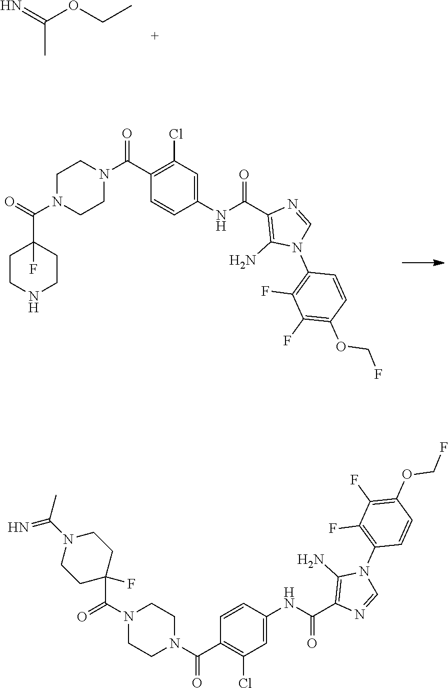

To solution of 5-amino-N-[3-chloro-4-[4-(4-fluoroisonipecotoyl)piperazine-1-carbonyl]phenyl]-1-[2,3-difluoro-4-(fluoromethoxy)phenyl]imidazole-4-carboxamide. 1:2 2,2,2-trifluoroacetic acid (Example Compound 34, 25 mg, 28 μmol, 1.0 eq) in N,N-dimethylformamide (1 mL) was added Et₃N (15.42 mg, 21.24 μL, 152.4 μmol, 5.500 eq) followed by acetimidic acid ethyl ester hydrochloride (6.85 mg, 55.4 μmol, 2.00 eq) at ambient temperature. The mixture was stirred overnight at ambient temperature. Then, the mixture was heated up to 50° C. and stirred for 1 h. Again acetimidic acid ethyl ester hydrochloride (6.85 mg, 55.4 μmol, 2.00 eq) and Et₃N (5.61 mg, 7.73 μL, 55.4 μmol, 2.00 eq) were added at ambient temperature. The reaction was then heated at 50° C. for 1 h, then at 60° C. for 1 h. Then, sodium bicarbonate (12.8 mg, 152 μmol, 5.50 eq) was added at ambient temperature. Then again acetimidic acid ethyl ester hydrochloride (6.85 mg, 55.4 μmol, 2.00 eq) was added and the reaction stirred at ambient temperature. Then Na₂CO₃ (2.94 mg, 27.7 μmol, 1.00 eq) was added and stirring continued at ambient temperature overnight. Then 1,4-dioxane (1 mL) and saturated aqueous NaHCO₃ solution (0.5 mL) were added and stirring continued. Then a solution of acetimidic acid ethyl ester hydrochloride (17.12 mg, 138.6 μmol, 5.000 eq) in saturated aqueous NaHCO₃ solution (0.50 mL) and 1,4-dioxane (500 μL) were added at ambient temperature and the mixture stirred over weekend at ambient temperature. The reaction mixture was filtered, washed with MeOH (5 ml), and the obtained solution concentrated under reduced pressure.

To a solution of acetimidic acid ethyl ester hydrochloride (68.49 mg, 554.2 μmol, 20.00 eq) in 1,4-dioxane (1 mL) and saturated aqueous NaHCO₃ solution (1 mL) was added a suspension of the above residue in saturated aqueous NaHCO₃ solution (1 mL) and 1,4-dioxane (1 mL). The resulting suspension was stirred at ambient temperature for 3 h. The mixture was filtered and washed with MeOH (5 ml), and the obtained solution concentrated under reduced pressure. The residue was suspended in DMSO and H₂O: Acetonitrile=9:1, filtered, and the obtained solution purified by preparative reverse phase chromatography (Phenomenex Gemini-NX 5u 110A, I:100 mm, dia: 30 mm) using acetonitrile/water (+0.05% TFA) as eluent. The corresponding fractions were combined and lyophilized to afford the title compound (14 mg, 55% yield). MS: m/z=723.4 [M+HCOO]⁻, ESI neg.

### Compound 90

### N-[4-[4-(1-amidino-4-fluoro-isonipecotoyl)piperazine-1-carbonyl]-3-chlorophenyl]-5-amino-1-[2,3-difluoro-4-(fluoromethoxy)phenyl]imidazole-4-carboxamide 1:1.3 2,2,2-trifluoroacetic acid

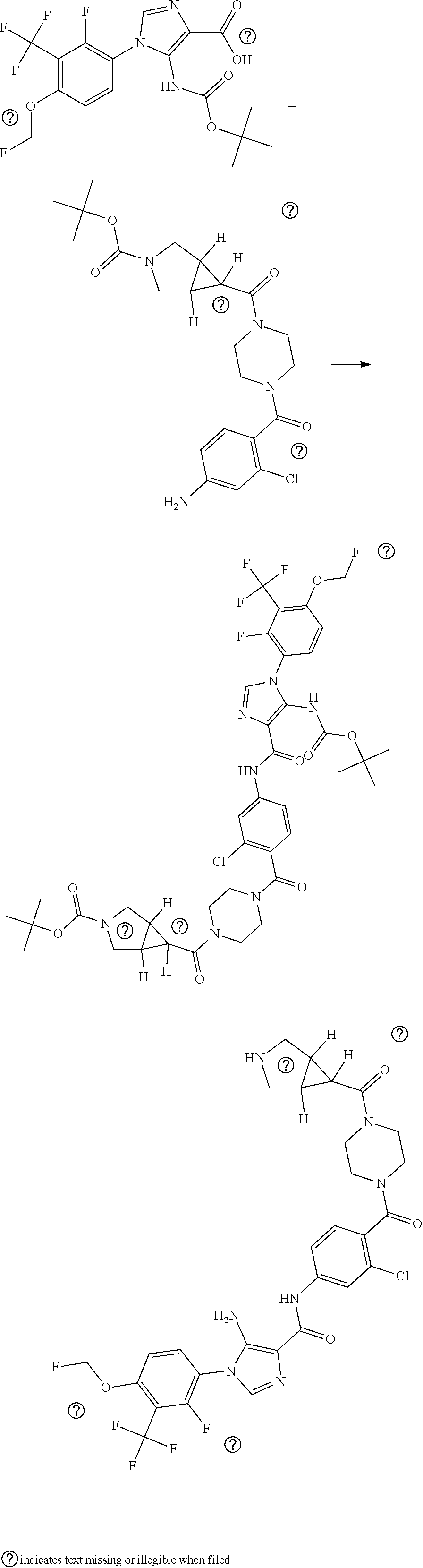

To a solution of 5-(tert-butoxycarbonylamino)-1-[2-fluoro-4-(fluoromethoxy)-3-(trifluoromethyl)phenyl]imidazole-4-carboxylic acid (intermediate 8, 41 mg, 91 μmol, 1.0 eq) and (1R,5S,6r)-6-[4-(4-amino-2-chlorobenzoyl)piperazine-1-carbonyl]-3-azabicyclo[3.1.0]hexane-3-carboxylic acid tert-butyl ester (intermediate 22, 47 mg, 100 μmol, 1.10 eq) in dichloromethane (1.5 mL) was added 1-methyl-1 h-imidazole (29 μL, 360 μmol, 4.0 eq). The reaction mixture was cooled to 0° C., then n-propylphosphonic acid anhydride, cyclic trimer (107 μL, 182 μmol, 2.00 eq) was added and the reaction stirred 20 min at 0° C. Then the reaction was heated to 40° C. for 5 hours. At room temperature were added again 1-methyl-1H-imidazole (29 μL, 360 μmol, 4.0 eq) and n-propylphosphonic acid anhydride, cyclic trimer (107 μL, 182 μmol, 2.00 eq) and the reaction was stirred overnight at 40° C. The reaction mixture was concentrated under reduced pressure. The residue was dissolved in DMSO and purified by preparative reverse phase HPLC (column: Phenomenex Gemini-NX 5u 110A, I:100 mm, dia 30 mm) using water containing 0.05% TFA/acetonitrile as eluent. The corresponding fractions were combined and lyophilized.

The intermediate was dissolved in dichloromethane (2 mL) and TFA (560 μL, 7.28 mmol, 80.0 eq) was added and the reaction stirred 4 hours at ambient temperature. The reaction was concentrated under reduced pressure, the residue dissolved in DMSO and then purified by preparative reverse phase HPLC (column: Phenomenex Gemini-NX 5u 110A, I:100 mm, dia 30 mm) using water containing 0.05% TFA/acetonitrile as eluent. The corresponding fractions were combined and lyophilized to afford the title compound (23 mg, 31% yield) as off-white lyoph. Solid. MS: m/z=712.6 [M+HCOO]⁻, ESI neg. ¹⁹F NMR confirmed ratio of parent to TFA as 1:1.3.

The following compounds were prepared in analogy to the procedure above starting from 5-(tert-butoxycarbonylamino)-1-[2-(difluoromethyl)-3-fluoro-4-(fluoromethoxy)phenyl]imidazole-4-carboxylic acid (intermediate 6).

| Compound No | Reagents | MS |
|---|---|---|
| 91 | (4-amino-2-chloro-phenyl)-[4- | m/z = 737.3 |
| | [(3aS,6aR)-2-methyl-1,3,3a,4,6,6a- | [M + HCOO]−, |
| | hexahydropyrrolo[3,4-c]pyrrole-5- | ESI neg |
| | carbonyl]piperazino]methanone | |
| | (intermediate 23) | |
| 92 | (3aR,6aS)-2-[4-(4-amino-2-chloro- | m/z = 723.3 |
| | benzoyl)piperazine-1-carbonyl]- | [M + HCOO]−, |
| | 1,3,3a,4,6,6a-hexahydropyrrolo[3,4- | ESI neg |
| | c]pyrrole-5-carboxylic acid tert- | |
| | butyl ester (intermediate 24) | |
| 93 | (4-amino-2-chloro-phenyl)-[4-(4- | m/z = 711.3 |
| | methylpiperazine-1-carbonyl)pi- | [M + HCOO]−, |
| | perazino]methanone (intermediate 25) | ESI neg |

The following compounds were prepared in analogy to the procedure above starting from 5-(tert-butoxycarbonylamino)-1-[4-(difluoromethoxy)-2,3-difluoro-phenyl]imidazole-4-carboxylic acid (intermediate 9).

| Compound No | Reagents | MS |
|---|---|---|
| 94 | 4-[4-(4-amino-2-chloro- | m/z = 683.2 |
| | benzoyl)piperazine-1- | [M + HCOO]−, |
| | carbonyl]piperazine-1- | ESI neg |
| | carboxylic acid tert- | |
| | butyl ester (Intermediate 26) | |
| 95 | 4-[1-(4-amino-2-chloro- | m/z = 682.3 |
| | benzoyl)isonipecotoyl]piperazine- | [M + HCOO]−, |
| | 1-carboxylic acid tert-butyl | ESI neg |
| | ester (intermediate 18), | |
| | extraction using 2M aq. | |
| | Na2CO3/dichloromethane | |
| | after deprotection | |
| 96 | (1R,5S,6r)-6-[4-(4-amino- | m/z = 680.2 |
| | 2-chloro-benzoyl)piperazine- | [M + HCOO]−, |
| | 1-carbonyl]-3- | ESI neg |
| | azabicyclo[3.1.0]hexane- | |
| | 3-carboxylic acid tert-butyl | |
| | ester (intermediate 22) | |

### Compound 97

### N-[4-[4-(1-amidino-4-fluoro-isonipecotoyl)piperazine-1-carbonyl]-3-chloro-phenyl]-5-amino-1-[2,3-difluoro-4-(fluoromethoxy)phenyl]imidazole-4-carboxamide. 1:2 2,2,2-trifluoroacetic acid

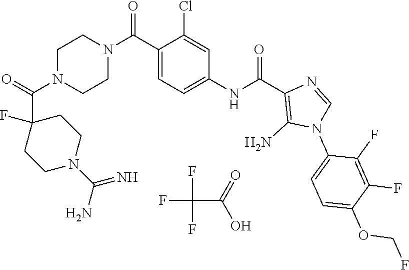 [SMILES: N=C(N)N1CCC(F)(C(=O)N2CCN(C(=O)c3ccc(NC(=O)c4ncn(-c5ccc(OCF)c(F)c5F)c4N)cc3Cl)CC2)CC1 ; O=C(O)C(F)(F)F]

### Step 1: N-[[4-[4-[4-[[5-amino-1-[2,3-difluoro-4-(fluoromethoxy)phenyl]imidazole-4-carbonyl]amino]-2-chloro-benzoyl]piperazine-1-carbonyl]-4-fluoro-piperidino]-(tert-butoxycarbonylamino)methylene]carbamic acid tert-butyl ester 0.1:1 2,2,2-trifluoroacetic acid

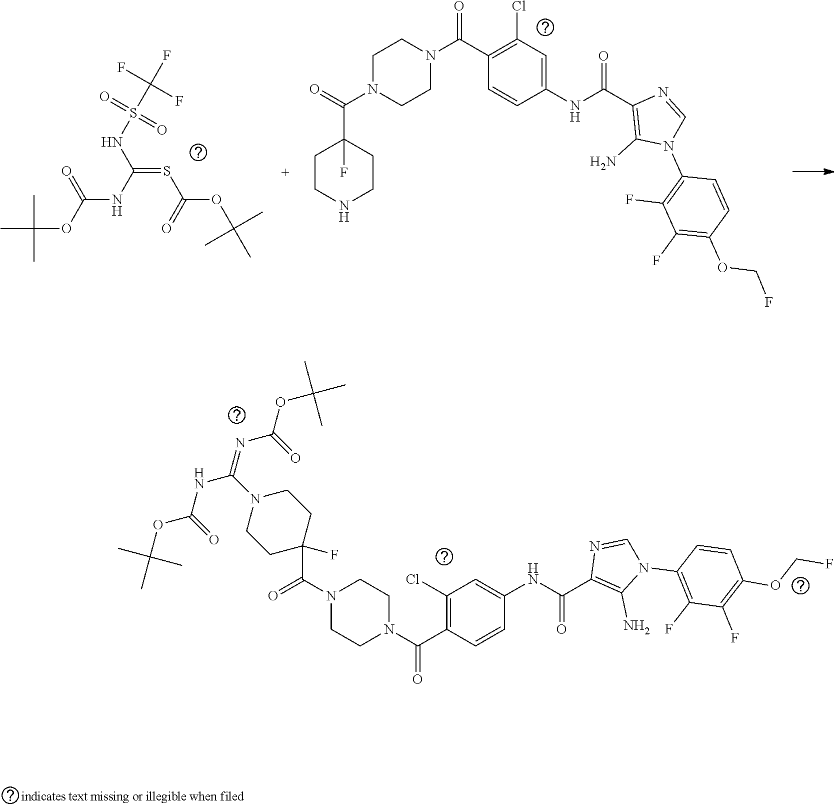

To a solution of 1,3-di-boc-2-(trifluoromethylsulfonyl) guanidine (260 mg, 665 μmol, 20.0 eq) in 1,4-dioxane (500 μL) was added a suspension of 5-amino-N-[3-chloro-4-[4-(4-fluoroisonipecotoyl)piperazine-1-carbonyl]phenyl]-1-[2,3-difluoro-4-(fluoromethoxy)phenyl]imidazole-4-carboxamide;2,2,2-trifluoroacetic acid (intermediate Compound 34, 30 mg, 33 μmol, 1.0 eq) in saturated aqueous NaHCO₃ solution (0.5 mL), and 1,4-dioxane (2 mL) was then used to transfer remains. The resulting suspension was stirred at room temperature overnight. The reaction mixture was concentrated under reduced pressure and the residue purified via reverse phase chromatography (Phenomenex Gemini-NX 5u 110A, I:100 mm, dia: 30 mm/100×30 mm, gradient acetonitrile/water (+0.05% TFA)=10:90 to 50:50). The corresponding fractions were combined and lyophilized to afford the title compound (13 mg, 39% yield). MS: m/z=878.7 [M−H]⁻, ESI neg.

### Step 2: N-[4-[4-(1-amidino-4-fluoro-isonipecotoyl)piperazine-1-carbonyl]-3-chloro-phenyl]-5-amino-1-[2,3-difluoro-4-(fluoromethoxy)phenyl]imidazole-4-carboxamide. 1:2 2,2,2-trifluoroacetic acid

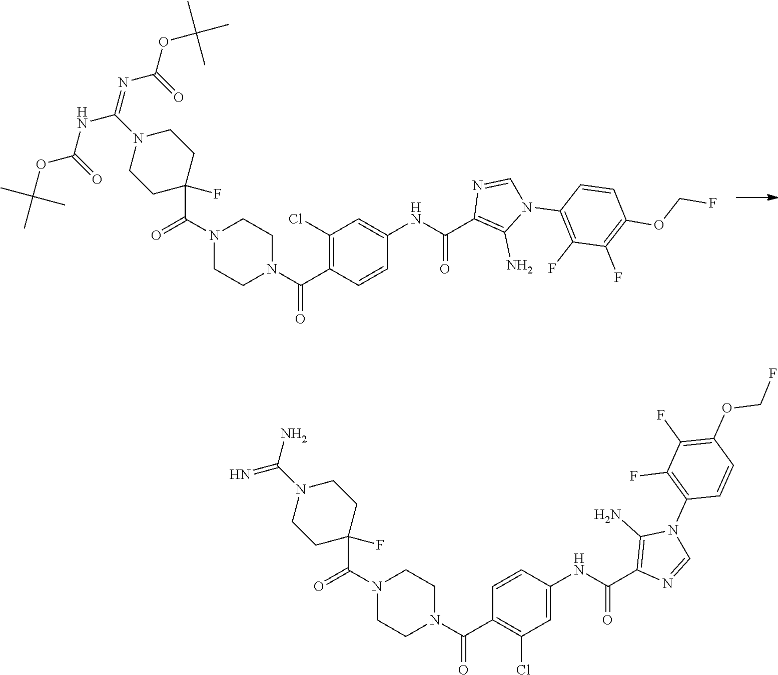 [SMILES: CC(C)(C)OC(=O)/N=C(/NC(=O)OC(C)(C)C)N1CCC(F)(C(=O)N2CCN(C(=O)c3ccc(NC(=O)c4ncn(-c5ccc(OCF)c(F)c5F)c4N)cc3Cl)CC2)CC1 ; N=C(N)N1CCC(F)(C(=O)N2CCN(C(=O)c3ccc(NC(=O)c4ncn(-c5ccc(OCF)c(F)c5F)c4N)cc3Cl)CC2)CC1]

N-[[4-[4-[4-[[5-amino-1-[2,3-difluoro-4-(fluoromethoxy)phenyl]imidazole-4-carbonyl]amino]-2-chlorobenzoyl]piperazine-1-carbonyl]-4-fluoro-piperidino]-(tert-butoxycarbonylamino)methylene]carbamic acid tert-butyl ester 0.1:1 2,2,2-trifluoroacetic acid (13 mg, 0.013 mmol, 1.000 eq) was combined with dichloromethane (1 mL) and TFA (50 μL, 647 μmol, 50 eq) was added. The mixture was stirred at ambient temperature overnight. Then, again TFA (50 μL, 650 μmol, 50 eq) was added and stirring continued for 1 hour. The reaction mixture was concentrated under reduced pressure and the residue purified by prep-HPLC (Phenomenex Gemini-NX 5u 110A, I:100 mm, dia: 30 mm/100×30 mm, gradient acetonitrile/water (+0.0 5% TFA)=10:90 to 30:70). The corresponding fractions were combined and lyophilized to afford the title compound (4.6 mg, 38% yield). MS: m/z=724.4 [M+HCOO]⁻, ESI neg.

### Compound 98

### 5-amino-N-[4-[4-[(1R,3s,5S,6r)-3-aminobicyclo[3.1.0]hexane-6-carbonyl]piperazine-1-carbonyl]-3-chlorophenyl]-1-[2,3-difluoro-4-(fluoromethoxy)phenyl]imidazole-4-carboxamide 0.1:1 2,2,2-trifluoroacetic acid

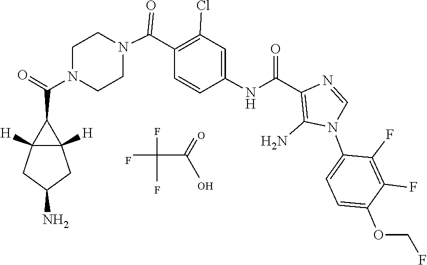

### Step 1: N-[5-[[4-[4-[(1R,3s,5S,6r)-3-(benzyloxycarbonylamino)bicyclo[3.1.0]hexane-6-carbonyl]piperazine-1-carbonyl]-3-chlorophenyl]carbamoyl]-3-[2,3-difluoro-4-(fluoromethoxy)phenyl]imidazol-4-yl]carbamic acid tert-butyl ester

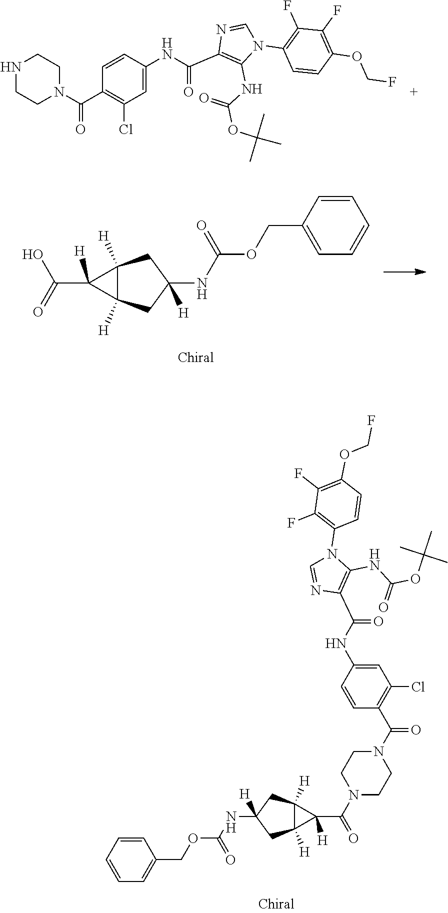 [SMILES: CC(C)(C)OC(=O)Nc1c(C(=O)Nc2ccc(C(=O)N3CCNCC3)c(Cl)c2)ncn1-c1ccc(OCF)c(F)c1F ; O=C(N[C@@H]1C[C@@H]2[C@H](C1)[C@H]2C(=O)O)OCc1ccccc1 ; CC(C)(C)OC(=O)Nc1c(C(=O)Nc2ccc(C(=O)N3CCN(C(=O)[C@H]4[C@@H]5C[C@@H](NC(=O)OCc6ccccc6)C[C@@H]54)CC3)c(Cl)c2)ncn1-c1ccc(OCF)c(F)c1F]

Under argon atmosphere N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester;2,2,2-trifluoroacetic acid (intermediate 11, 100 mg, 137 μmol, 1.00 eq) was dissolved in N,N-dimethylformamide, extra dry (3 mL), then DIEA (70.79 mg, 95.66 μL, 547.7 μmol, 4.000 eq) was added, followed by (1R,3s,5S,6r)-3-(benzyloxycarbonylamino)bicyclo[3.1.0]hexane-6-carboxylic acid (intermediate 30, 41.47 mg, 150.6 μmol, 1.100 eq) and HATU (57.27 mg, 150.6 μmol, 1.100 eq). The reaction was stirred at ambient temperature for 20 h. The reaction was poured onto water and extracted with ethyl acetate. The combined organic layers were washed with brine, dried over MgSO₄, filtered and evaporated. The residue was purified by silica gel chromatography using dichloromethane and (CH₂Cl₂:MeOH=9:1+0.25% NH₃) as eluents. The corresponding fractions were combined and concentrated under reduced pressure to afford the title compound (97 mg, 81% yield). MS: m/z=866.6 [M+H]⁺, ESI pos.

### Step 2: N-[(1R,3s,5S,6r)-6-[4-[4-[[5-amino-1-[2,3-difluoro-4-(fluoromethoxy)phenyl]imidazole-4-carbonyl]amino]-2-chlorobenzoyl]piperazine-1-carbonyl]-3-bicyclo[3.1.0]hexanyl]carbamic acid benzyl ester 0.1:1 2,2,2-trifluoroacetic acid

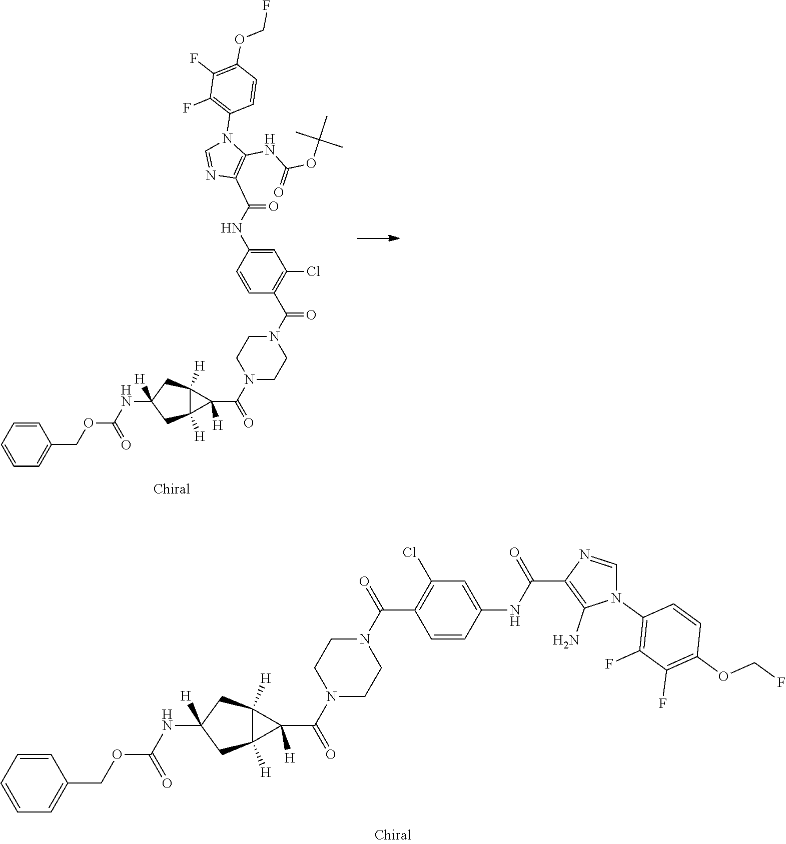 [SMILES: CC(C)(C)OC(=O)Nc1c(C(=O)Nc2ccc(C(=O)N3CCN(C(=O)[C@H]4[C@@H]5C[C@@H](NC(=O)OCc6ccccc6)C[C@@H]54)CC3)c(Cl)c2)ncn1-c1ccc(OCF)c(F)c1F ; Nc1c(C(=O)Nc2ccc(C(=O)N3CCN(C(=O)[C@H]4[C@@H]5C[C@@H](NC(=O)OCc6ccccc6)C[C@@H]54)CC3)c(Cl)c2)ncn1-c1ccc(OCF)c(F)c1F]

N-[5-[[4-[4-[(1R,3s,5S,6r)-3-(benzyloxycarbonylamino)bicyclo[3.1.0]hexane-6-carbonyl]piperazine-1-carbonyl]-3-chlorophenyl]carbamoyl]-3-[2,3-difluoro-4-(fluoromethoxy)phenyl]imidazol-4-yl]carbamic acid tert-butyl ester (35 mg, 40 μmol, 1.0 eq) was dissolved in dichloromethane (1 mL) and TFA (158 μL, 2.05 mmol, 50.0 eq) was added. The mixture was then stirred at ambient temperature for 20 h. The mixture was concentrated under reduced pressure and the obtained material purified by reverse phase chromatography (Phenomenex Gemini-NX 5u 110A, I:100 mm, dia: 30 mm/100×30 mm, gradient acetonitrile/water (+0.05% TFA)=10:90 to 70:30). The corresponding fractions were combined and lyophilized to afford the title compound (24 mg, 67% yield). MS: m/z=766.7 [M+H]⁺, ESI pos.

### Step 3: 5-amino-N-[4-[4-[(1R,3s,5S,6r)-3-aminobicyclo[3.1.0]hexane-6-carbonyl]piperazine-1-carbonyl]-3-chlorophenyl]-1-[2,3-difluoro-4-(fluoromethoxy)phenyl]imidazole-4-carboxamide 0.1:1 2,2,2-trifluoroacetic acid

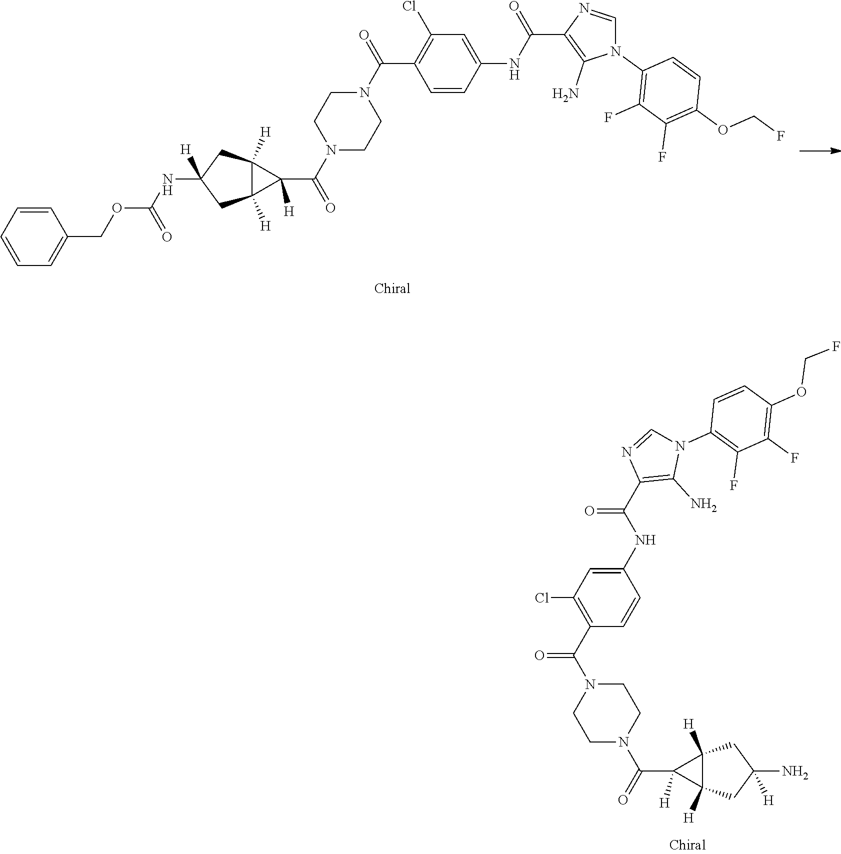 [SMILES: Nc1c(C(=O)Nc2ccc(C(=O)N3CCN(C(=O)[C@H]4[C@@H]5C[C@@H](NC(=O)OCc6ccccc6)C[C@@H]54)CC3)c(Cl)c2)ncn1-c1ccc(OCF)c(F)c1F ; Nc1c(C(=O)Nc2ccc(C(=O)N3CCN(C(=O)[C@H]4[C@@H]5C[C@@H](N)C[C@@H]54)CC3)c(Cl)c2)ncn1-c1ccc(OCF)c(F)c1F]

N-[(1S,3s,5R,6r)-6-[4-[4-[[5-amino-1-[2,3-difluoro-4-(fluoromethoxy)phenyl]imidazole-4-carbonyl]amino]-2-chlorobenzoyl]piperazine-1-carbonyl]-3-bicyclo[3.1.0]hexanyl]carbamic acid benzyl ester;2,2,2-trifluoroacetic acid (19 mg, 21 μmol, 1.0 eq) was dissolved in methanol (1 mL) and 10% palladium on carbon (6.8 mg, 6.4 μmol, 0.30 eq) was added. The reaction mixture was stirred under hydrogen atmosphere at ambient temperature for 18 h. The reaction mixture was then filtered, and the obtained solution concentrated under reduced pressure to afford the title compound (14 mg, 87% yield). MS: m/z=632.4 [M+H]+, ESI pos.

### Compound 99

### 3-[[4-[[5-amino-1-[2,3-difluoro-4-(fluoromethoxy)phenyl]imidazole-4-carbonyl]amino]-2-chlorobenzoyl]amino]azetidine-1-carboxylic acid benzyl ester 0.1:0.5 2,2,2-trifluoroacetic acid

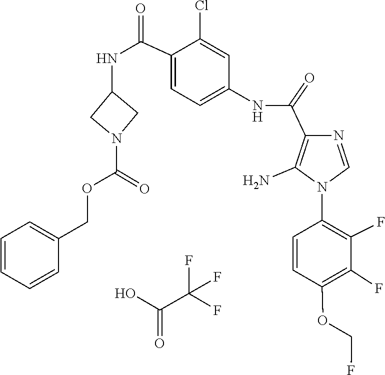 [SMILES: Nc1c(C(=O)Nc2ccc(C(=O)NC3CN(C(=O)OCc4ccccc4)C3)c(Cl)c2)ncn1-c1ccc(OCF)c(F)c1F ; O=C(O)C(F)(F)F]

### Step 1: 3-[[4-[[5-(tert-butoxycarbonylamino)-1-[2,3-difluoro-4-(fluoromethoxy)phenyl]imidazole-4-carbonyl]amino]-2-chlorobenzoyl]amino]azetidine-1-carboxylic acid benzyl ester

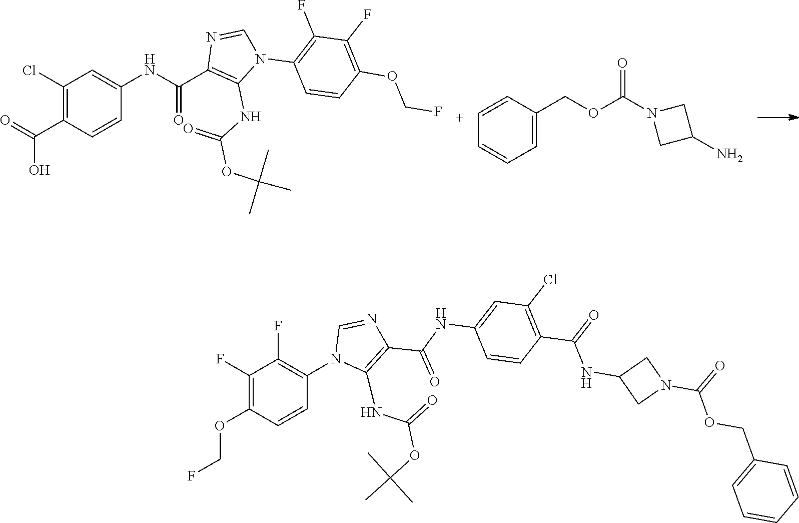

Under argon atmosphere 4-[[5-(tert-butoxycarbonylamino)-1-[2,3-difluoro-4-(fluoromethoxy)phenyl]imidazole-4-carbonyl]amino]-2-chlorobenzoic acid (intermediate 10, 250 mg, 462 μmol, 1.00 eq) was dissolved in N,N-dimethylformamide (5 mL), then DIEA (239 mg, 323 μL, 1.85 mmol, 4.00 eq) was added, followed by HATU (193 mg, 508 μmol, 1.10 eq) and 3-aminoazetidine-1-carboxylic acid benzyl ester (105 mg, 508 μmol, 1.10 eq). The reaction was stirred at ambient temperature for 17 hours. The mixture was concentrated under reduced pressure. The residue was dissolved in DMSO and then purified by preparative reverse phase HPLC (column: Phenomenex Gemini-NX 5u 110A, I:100 mm, dia 30 mm) using water containing 0.05% TFA/acetonitrile as eluent. The corresponding fractions were collected and lyophilized to afford the title compound (332 mg, 96% yield). MS: 729.6 [M+H]⁺, ESI pos.

### Step 2: 3-[[4-[[5-amino-1-[2,3-difluoro-4-(fluoromethoxy)phenyl]imidazole-4-carbonyl]amino]-2-chlorobenzoyl]amino]azetidine-1-carboxylic acid benzyl ester 0.1:0.5 2,2,2-trifluoroacetic acid

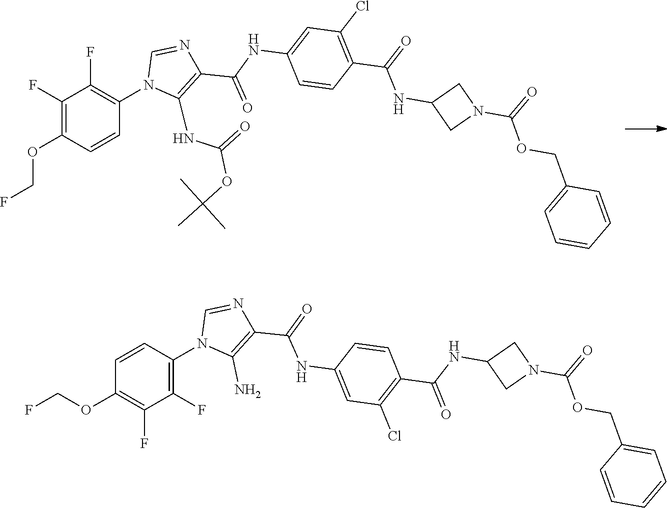 [SMILES: CC(C)(C)OC(=O)Nc1c(C(=O)Nc2ccc(C(=O)NC3CN(C(=O)OCc4ccccc4)C3)c(Cl)c2)ncn1-c1ccc(OCF)c(F)c1F ; Nc1c(C(=O)Nc2ccc(C(=O)NC3CN(C(=O)OCc4ccccc4)C3)c(Cl)c2)ncn1-c1ccc(OCF)c(F)c1F]

3-[[4-[[5-(tert-butoxycarbonylamino)-1-[2,3-difluoro-4-(fluoromethoxy)phenyl]imidazole-4-carbonyl]amino]-2-chlorobenzoyl]amino]azetidine-1-carboxylic acid benzyl ester (16 mg, 21 μmol, 1.0 eq) was dissolved in dichloromethane (1.07 mL) and TFA (122 mg, 83 μL, 1.08 mmol, 50.0 eq) was added. After 16 hours TFA (122 mg, 83 μL, 1.08 mmol, 50.0 eq) was added again and stirring continued for 1 hour. The reaction was concentrated under reduced pressure, the residue dissolved in DMSO, and purified by preparative reverse phase HPLC (column: Phenomenex Gemini-NX 5u 110A, I:100 mm, dia 30 mm) using water containing 0.05% TFA/acetonitrile as eluent. The corresponding fractions were combined and lyophilized to afford the title compound (6 mg, 40% yield). MS: 629.5 [M+H]⁺, ESI pos.

### Compound 100

### 5-amino-N-[4-[4-[(1R,5S,6r)-3-azabicyclo[3.1.0]hexane-6-carbonyl]piperazine-1-carbonyl]-3-chlorophenyl]-2-chloro-1-[2,3-difluoro-4-(fluoromethoxy)phenyl]imidazole-4-carboxamide 0.1:1 2,2,2-trifluoroacetic acid

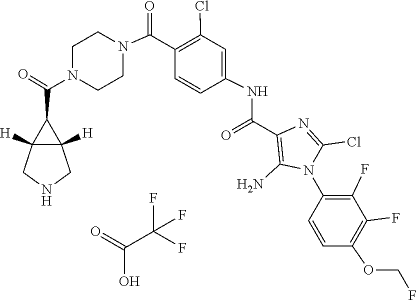 [SMILES: Nc1c(C(=O)Nc2ccc(C(=O)N3CCN(C(=O)[C@H]4[C@@H]5CNC[C@@H]54)CC3)c(Cl)c2)nc(Cl)n1-c1ccc(OCF)c(F)c1F ; O=C(O)C(F)(F)F]

### Step 1: 5-amino-2-chloro-1-[2,3-difluoro-4-(fluoromethoxy)phenyl]imidazole-4-carboxylic acid ethyl ester

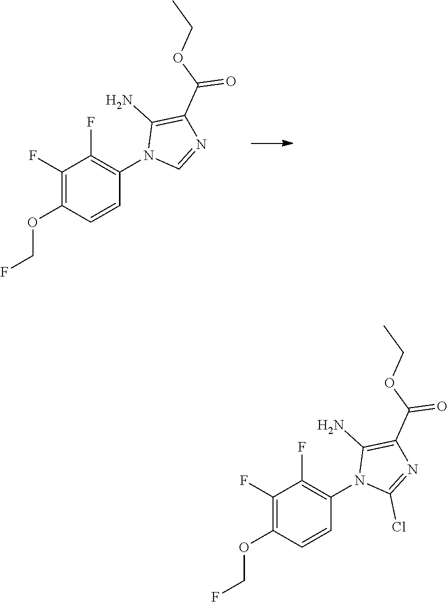 [SMILES: CCOC(=O)c1ncn(-c2ccc(OCF)c(F)c2F)c1N ; CCOC(=O)c1nc(Cl)n(-c2ccc(OCF)c(F)c2F)c1N]

To a solution of 5-amino-1-[2,3-difluoro-4-(fluoromethoxy)phenyl]imidazole-4-carboxylic acid ethyl ester (intermediate 2, 680 mg, 2.11 mmol, 1.000 eq) in N,N-dimethylformamide (7 mL) was added a solution of N-chlorosuccinimide (310.49 mg, 2.33 mmol, 1.100 eq) in N,N-dimethylformamide (7 mL) and the reaction stirred 3 h 40 min at ambient temperature. The reaction was diluted with water and extracted 3 times with ethyl acetate. The combined organic layers were washed with water and brine, dried over magnesium sulfate, filtered and concentrated under reduced pressure. The residue was suspended in TBME, filtered, and collection of the solid afforded the title compound (478 mg, 55.6% yield) with a purity of 86% (contains 14% of starting material). The material was used without further purification. MS: m/z=350.1 [M+H]⁺, ESI pos.

### Step 2: 5-[bis(tert-butoxycarbonyl)amino]-2-chloro-1-[2,3-difluoro-4-(fluoromethoxy)phenyl]imidazole-4-carboxylic acid ethyl ester

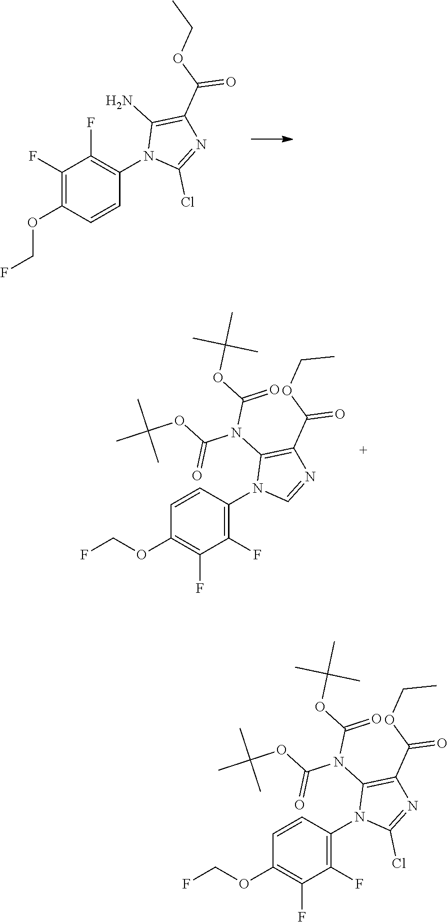 [SMILES: CCOC(=O)c1nc(Cl)n(-c2ccc(OCF)c(F)c2F)c1N ; CCOC(=O)c1ncn(-c2ccc(OCF)c(F)c2F)c1N(C(=O)OC(C)(C)C)C(=O)OC(C)(C)C ; CCOC(=O)c1nc(Cl)n(-c2ccc(OCF)c(F)c2F)c1N(C(=O)OC(C)(C)C)C(=O)OC(C)(C)C]

To a mixture of 5-amino-2-chloro-1-[2,3-difluoro-4-(fluoromethoxy)phenyl]imidazole-4-carboxylic acid ethyl ester (535 mg, 1.32 mmol, 1.00 eq) and 4-dimethylaminopyridine (32.15 mg, 263.2 μmol, 0.200 eq) in dichloromethane (10 mL) was added Et₃N (399.42 mg, 550.16 μL, 3.95 mmol, 3.000 eq) and di-tert-butyl dicarbonate (717.89 mg, 763.71 μL, 3.29 mmol, 2.500 eq). The reaction mixture was heated to 30° C. for 1.5 h. The reaction was diluted with water and extracted 3 times with dichloromethane. The combined organic layers were washed with brine, dried over magnesium sulfate, filtered and concentrated under reduced pressure. The residue as a drypack on isolute HM-N adsorbent was purified by silica gel chromatography using heptane:ethyl acetate as eluent to afford the title compound (598 mg, 81% yield) LC-MS: 550.2 [M+H]⁺, ESI pos. MS: m/z=550.2 [M+H]⁺, ESI pos.

### Step 3: 5-(tert-butoxycarbonylamino)-2-chloro-1-[2,3-difluoro-4-(fluoromethoxy)phenyl]imidazole-4-carboxylic acid

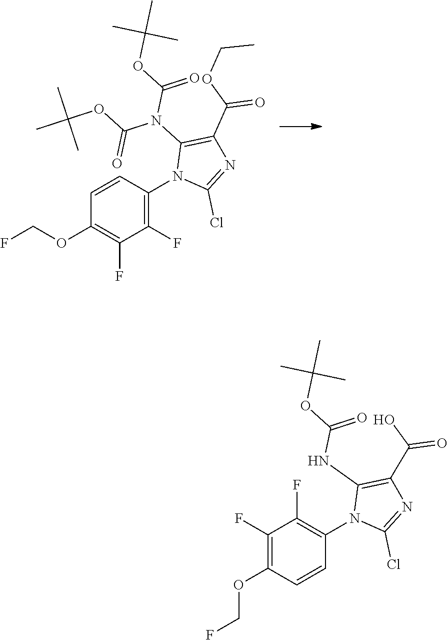 [SMILES: CCOC(=O)c1nc(Cl)n(-c2ccc(OCF)c(F)c2F)c1N(C(=O)OC(C)(C)C)C(=O)OC(C)(C)C ; CC(C)(C)OC(=O)Nc1c(C(=O)O)nc(Cl)n1-c1ccc(OCF)c(F)c1F]

To a solution of 5-[bis(tert-butoxycarbonyl)amino]-2-chloro-1-[2,3-difluoro-4-(fluoromethoxy)phenyl]imidazole-4-carboxylic acid ethyl ester (598 mg, 1.07 mmol, 1.00 eq) in tetrahydrofuran (5 mL) was added water (5 mL) and lithium hydroxide monohydrate (983.84 mg, 23.45 mmol, 22.000 eq) and the mixture stirred 3 hours at room temperature. Then, the reaction was heated to 30° C. for 18 hours. Again lithium hydroxide monohydrate (983.84 mg, 23.45 mmol, 22.000 eq) was added and stirring continued for 4 h. The reaction was cooled to room temperature and was acidified with 3 M HCl and extracted with ethyl acetate. The combined organic layers were washed with brine, dried over MgSO₄, filtered and concentrated under reduced pressure to afford the title compound with a purity of 97% (465 mg, 100% yield). MS: m/z=366.0 [M+H−butene]⁺, ESI pos.

### Step 4: 4-[4-[[5-(tert-butoxycarbonylamino)-2-chloro-1-[2,3-difluoro-4-(fluoromethoxy)phenyl]imidazole-4-carbonyl]amino]-2-chlorobenzoyl]piperazine-1-carboxylic acid 9H-fluoren-9-ylmethyl ester

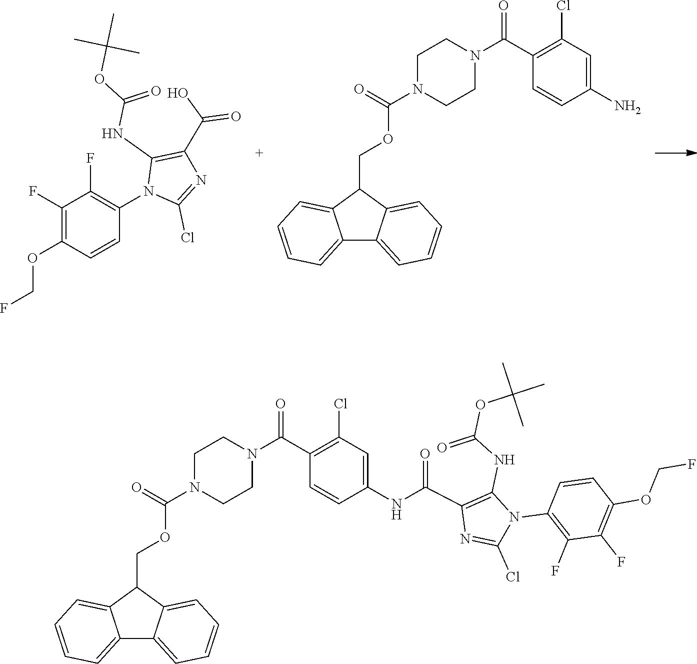 [SMILES: CC(C)(C)OC(=O)Nc1c(C(=O)O)nc(Cl)n1-c1ccc(OCF)c(F)c1F ; Nc1ccc(C(=O)N2CCN(C(=O)OCC3c4ccccc4-c4ccccc43)CC2)c(Cl)c1 ; CC(C)(C)OC(=O)Nc1c(C(=O)Nc2ccc(C(=O)N3CCN(C(=O)OCC4c5ccccc5-c5ccccc54)CC3)c(Cl)c2)nc(Cl)n1-c1ccc(OCF)c(F)c1F]

To a solution of 5-(tert-butoxycarbonylamino)-2-chloro-1-[2,3-difluoro-4-(fluoromethoxy)phenyl]imidazole-4-carboxylic acid (324 mg, 745 μmol, 1.00 eq) and DIEA (288.91 mg, 390.43 UL, 2.24 mmol, 3.000 eq) in N,N-dimethylformamide (2.89 mL) was added HATU (340 mg, 894 μmol, 1.20 eq). After 15 min a solution of 4-(4-amino-2-chlorobenzoyl)piperazine-1-carboxylic acid 9H-fluoren-9-ylmethyl ester (intermediate 28, 394.42 mg, 819.68 μmol, 1.100 eq) in N,N-dimethylformamide (2.89 mL) was added and the reaction stirred 3 days at ambient temperature. The reaction was diluted with water and extracted 3 times with ethyl acetate. The combined organic layers were washed with brine, dried over magnesium sulfate, filtered and concentrated under reduced pressure. The residue was purified by silica gel chromatography using dichloromethane/methanol+0.25% NH₄OH as eluent to afford the title compound (173 mg, 14.7% yield) with a purity of approx. 55%. This material was used in the next step without any further purification. MS: m/z=863.3 [M−H]⁻, ESI neg.

### Step 5: N-[2-chloro-5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester

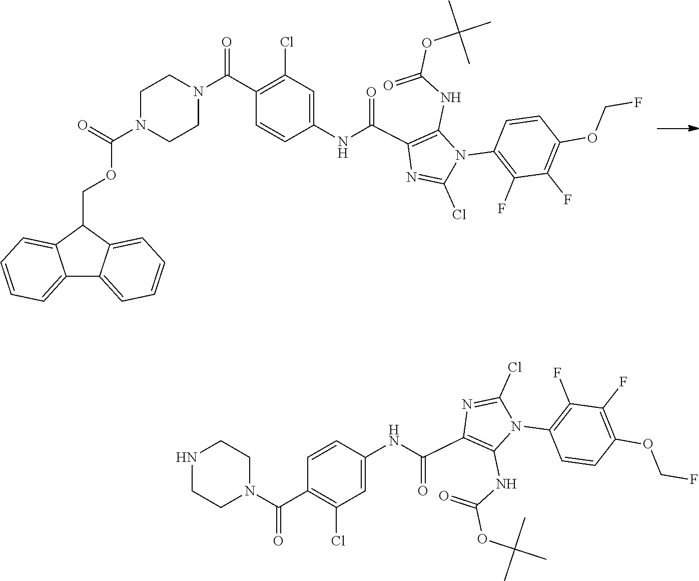 [SMILES: CC(C)(C)OC(=O)Nc1c(C(=O)Nc2ccc(C(=O)N3CCN(C(=O)OCC4c5ccccc5-c5ccccc54)CC3)c(Cl)c2)nc(Cl)n1-c1ccc(OCF)c(F)c1F ; CC(C)(C)OC(=O)Nc1c(C(=O)Nc2ccc(C(=O)N3CCNCC3)c(Cl)c2)nc(Cl)n1-c1ccc(OCF)c(F)c1F]

To a solution of 4-[4-[[5-(tert-butoxycarbonylamino)-2-chloro-1-[2,3-difluoro-4-(fluoromethoxy)phenyl]imidazole-4-carbonyl]amino]-2-chlorobenzoyl]piperazine-1-carboxylic acid 9H-fluoren-9-ylmethyl ester (173 mg, 0.110 mmol, 1.00 eq) in tetrahydrofuran (1 mL) was added 2 M dimethylamine in THF (146.73 mg, 164.87 L, 329.74 μmol, 3.000 eq) and the reaction stirred 16 hours at ambient temperature. The solvent was evaporated and the residue purified by silica gel chromatography using dichloromethane/methanol+0.25% NH₄OH as eluent to afford the title compound (65 mg, 91% yield). MS: m/z=643.3 [M+H]⁺, ESI pos.

### Step 6: 5-amino-N-[4-[4-[(1R,5S,6r)-3-azabicyclo[3.1.0]hexane-6-carbonyl]piperazine-1-carbonyl]-3-chlorophenyl]-2-chloro-1-[2,3-difluoro-4-(fluoromethoxy)phenyl]imidazole-4-carboxamide 0.1:1 2,2,2-trifluoroacetic acid

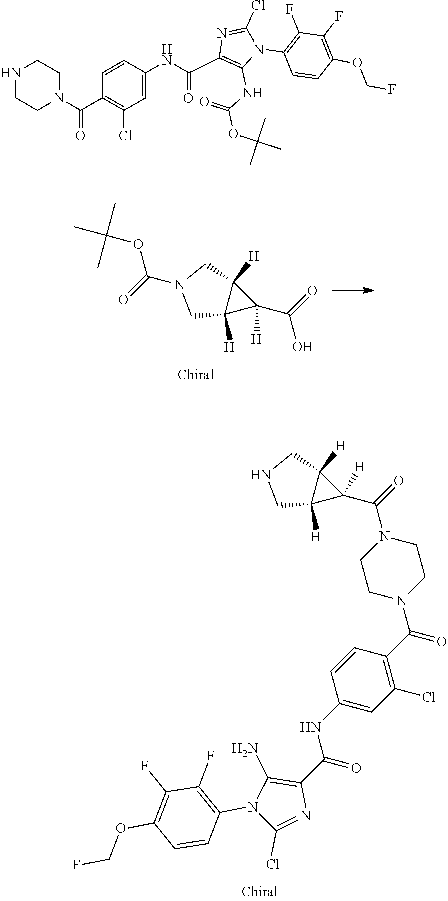 [SMILES: CC(C)(C)OC(=O)Nc1c(C(=O)Nc2ccc(C(=O)N3CCNCC3)c(Cl)c2)nc(Cl)n1-c1ccc(OCF)c(F)c1F ; CC(C)(C)OC(=O)N1C[C@@H]2[C@H](C1)[C@H]2C(=O)O ; Nc1c(C(=O)Nc2ccc(C(=O)N3CCN(C(=O)[C@H]4[C@@H]5CNC[C@@H]54)CC3)c(Cl)c2)nc(Cl)n1-c1ccc(OCF)c(F)c1F]

To a solution of (1R,5S,6r)-3-[(tert-butoxy) carbonyl]-3-azabicyclo[3.1.0]hexane-6-carboxylic acid (11.16 mg, 49.11 μmol, 1.200 eq) and DIEA (21.16 mg, 28.59 μL, 163.7 μmol, 4.000 eq) in N,N-dimethylformamide (500 μL) was added HATU (18.67 mg, 49.11 μmol, 1.200 eq) and the mixture stirred 2 hours at room temperature. Then, a solution of N-[2-chloro-5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester (31 mg, 41 μmol, 1.0 eq) in N,N-dimethylformamide (1 mL) was added and stirring continued for 1 h 20 min. The solvent was evaporated. The residue was dissolved in dichloromethane (1 mL) and TFA (466.63 mg, 315.29 μL, 4.09 mmol, 100.00 eq) was added and the reaction stirred 3 h at ambient temperature. The reaction was stored overnight in the refrigerator. Then, the solvent was evaporated, the residue dissolved in DMSO and purified by preparative reverse phase HPLC (column: Phenomenex Gemini-NX 5u 110A, I:100 mm, dia 30 mm) using water containing 0.05% TFA/acetonitrile as eluent. The corresponding fractions were combined and lyophilized to afford the title compound (23 mg, 73% yield). MS: 696.2 [M+HCOO]⁻, ESI neg.

### Compound 101

### 5-amino-N-[4-[4-[(1R,5S,6r)-3-azabicyclo[3.1.0]hexane-6-carbonyl]piperazine-1-carbonyl]-3-chlorophenyl]-2-chloro-1-[2-fluoro-4-(fluoromethoxy)phenyl]imidazole-4-carboxamide 0.1:1 2,2,2-trifluoroacetic acid

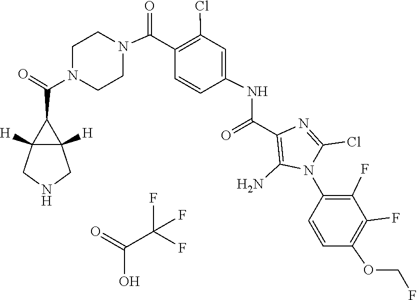 [SMILES: Nc1c(C(=O)Nc2ccc(C(=O)N3CCN(C(=O)[C@H]4[C@@H]5CNC[C@@H]54)CC3)c(Cl)c2)nc(Cl)n1-c1ccc(OCF)c(F)c1F ; O=C(O)C(F)(F)F]

### Step 1: 4-[4-[[5-(tert-butoxycarbonylamino)-1-[2-fluoro-4-(fluoromethoxy)phenyl]imidazole-4-carbonyl]amino]-2-chlorobenzoyl]piperazine-1-carboxylic acid 9H-fluoren-9-ylmethyl ester

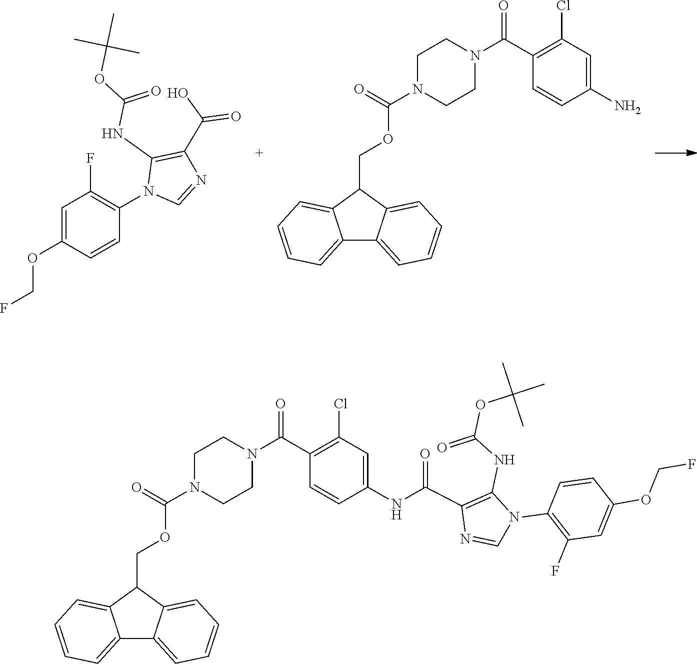 [SMILES: CC(C)(C)OC(=O)Nc1c(C(=O)O)ncn1-c1ccc(OCF)cc1F ; Nc1ccc(C(=O)N2CCN(C(=O)OCC3c4ccccc4-c4ccccc43)CC2)c(Cl)c1 ; CC(C)(C)OC(=O)Nc1c(C(=O)Nc2ccc(C(=O)N3CCN(C(=O)OCC4c5ccccc5-c5ccccc54)CC3)c(Cl)c2)ncn1-c1ccc(OCF)cc1F]

To a suspension of 5-(tert-butoxycarbonylamino)-1-[2-fluoro-4-(fluoromethoxy)phenyl]imidazole-4-carboxylic acid (intermediate 7, 300 mg, 812 μmol, 1.00 eq) in acetonitrile (5 mL) was added 1-methylimidazole (266.77 mg, 259 μL, 3.25 mmol, 4.000 eq). Then 4-(4-amino-2-chloro-benzoyl)piperazine-1-carboxylic acid 9H-fluoren-9-ylmethyl ester (intermediate 28, 562.85 mg, 1.22 mmol, 1.500 eq) and N,N,N′,N′-tetramethylchloroformamidinium hexafluorophosphate (TCFH) (341.87 mg, 1.22 mmol, 1.500 eq) were added and the reaction stirred 15 hours at ambient temperature. Again TCFH (227.92 mg, 812.3 μmol, 1.000 eq) and 1-methylimidazole (200.08 mg, 194.25 μL, 2.44 mmol, 3.000 eq) were added and stirring continued 4.5 h. Again, TCFH (227.92 mg, 812.3 μmol, 1.000 eq) was added and stirring continued for 15 h. The reaction mixture was filtered and the solid collected to afford the title compound (430 mg; 62% yield). MS: 811.5 [M−H]⁻, ESI neg.

The mother liquor was concentrated under reduced pressure. The residue was dissolved in DMSO and purified by preparative reverse phase chromatography (Phenomenex Gemini-NX 5u 110A, I:100 mm, dia: 30 mm/100×30 mm, eluents acetonitrile/water (+0.05% TFA). The corresponding fractions were collected and lyophilized to afford another batch of the title compound (TFA salt, 77 mg, 10% yield). MS: 811.4 [M−H]⁻, ESI neg.

Both batches were combined and used in the next step.

### Step 2: N-[5-[[3-chloro-4-(piperazine-1-carbonyl)phenyl]carbamoyl]-3-[2-fluoro-4-(fluoromethoxy)phenyl]imidazol-4-yl]carbamic acid tert-butyl ester

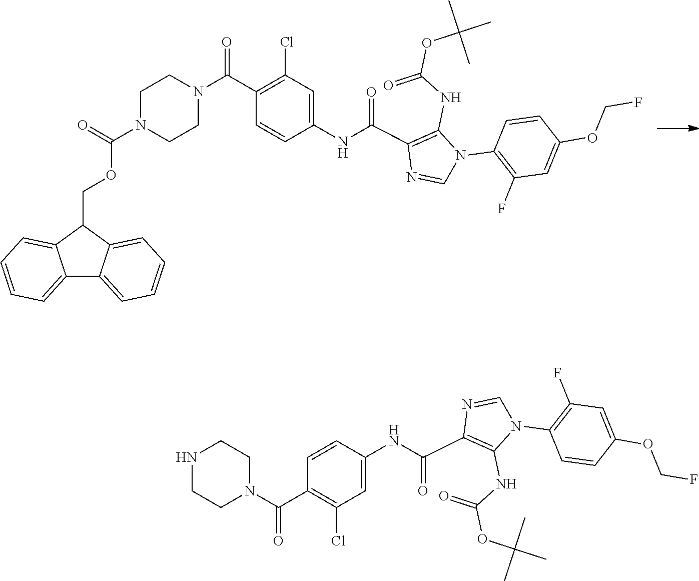 [SMILES: CC(C)(C)OC(=O)Nc1c(C(=O)Nc2ccc(C(=O)N3CCN(C(=O)OCC4c5ccccc5-c5ccccc54)CC3)c(Cl)c2)ncn1-c1ccc(OCF)cc1F ; CC(C)(C)OC(=O)Nc1c(C(=O)Nc2ccc(C(=O)N3CCNCC3)c(Cl)c2)ncn1-c1ccc(OCF)cc1F]

To a suspension of 4-[4-[[5-(tert-butoxycarbonylamino)-1-[2-fluoro-4-(fluoromethoxy)phenyl]imidazole-4-carbonyl]amino]-2-chlorobenzoyl]piperazine-1-carboxylic acid 9H-fluoren-9-ylmethyl ester (497 mg, 581 μmol, 1.00 eq) in tetrahydrofuran (5.11 mL) was added 2 M dimethylamine solution in THF (1.16 mL, 2.32 mmol, 4.00 eq) and the reaction stirred 17 h at ambient temperature. The solvent was evaporated and the residue as drypack on isolute HM-N adsorbent purified by silica gel chromatography using dichloromethane/methanol+0.25% NH₄OH as eluent to afford the title compound (341 mg, 98% yield). MS: 589.3 [M−H]⁻, ESI neg.

### Step 3: N-[2-chloro-5-[[3-chloro-4-(piperazine-1-carbonyl)phenyl]carbamoyl]-3-[2-fluoro-4-(fluoromethoxy)phenyl]imidazol-4-yl]carbamic acid tert-butyl ester 0.1:1 2,2,2-trifluoroacetic acid

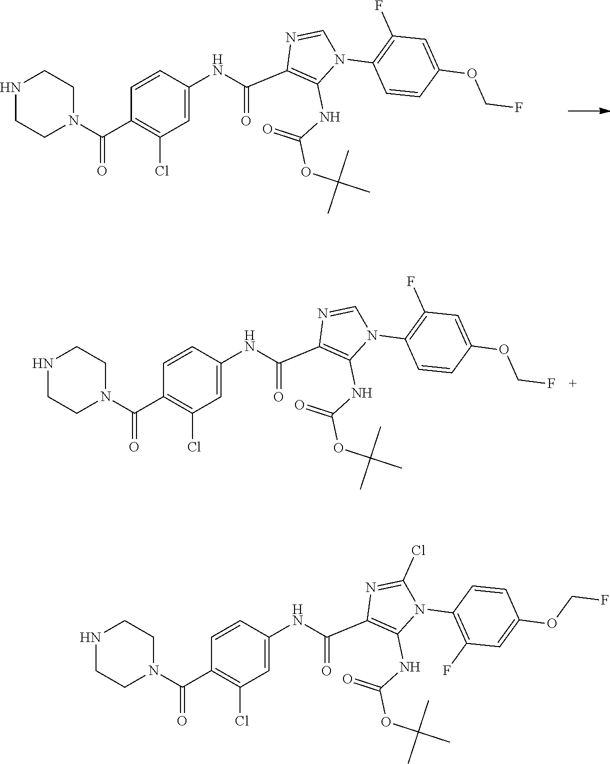 [SMILES: CC(C)(C)OC(=O)Nc1c(C(=O)Nc2ccc(C(=O)N3CCNCC3)c(Cl)c2)ncn1-c1ccc(OCF)cc1F ; CC(C)(C)OC(=O)Nc1c(C(=O)Nc2ccc(C(=O)N3CCNCC3)c(Cl)c2)ncn1-c1ccc(OCF)cc1F ; CC(C)(C)OC(=O)Nc1c(C(=O)Nc2ccc(C(=O)N3CCNCC3)c(Cl)c2)nc(Cl)n1-c1ccc(OCF)cc1F]

N-[5-[[3-chloro-4-(piperazine-1-carbonyl)phenyl]carbamoyl]-3-[2-fluoro-4-(fluoromethoxy)phenyl]imidazol-4-yl]carbamic acid tert-butyl ester (145 mg, 243 μmol, 1.000 eq) and N-chlorosuccinimide (38.92 mg, 291.5 μmol, 1.200 eq) were dissolved in N, N-dimethylformamide (2 mL) and the reaction stirred 6 h at ambient temperature. The reaction was diluted with water and stored overnight in the refrigerator. The reaction was extracted 3 times with ethyl acetate. The combined organic layers were washed with brine, dried over magnesium sulfate, filtered and concentrated under reduced pressure. The residue was dissolved in DMSO and purified by preparative reverse phase HPLC (column: Phenomenex Gemini-NX 5u 110A, I:100 mm, dia 30 mm) using water containing 0.05% TFA/acetonitrile as eluent. The corresponding fractions were combined and lyophilized to afford the title compound (23 mg, 12% yield). MS: m/z=625.4 [M+H]⁺, ESI pos.

### Step 4: 5-amino-N-[4-[4-[(1R,5S,6r)-3-azabicyclo[3.1.0]hexane-6-carbonyl]piperazine-1-carbonyl]-3-chloro-phenyl]-2-chloro-1-[2-fluoro-4-

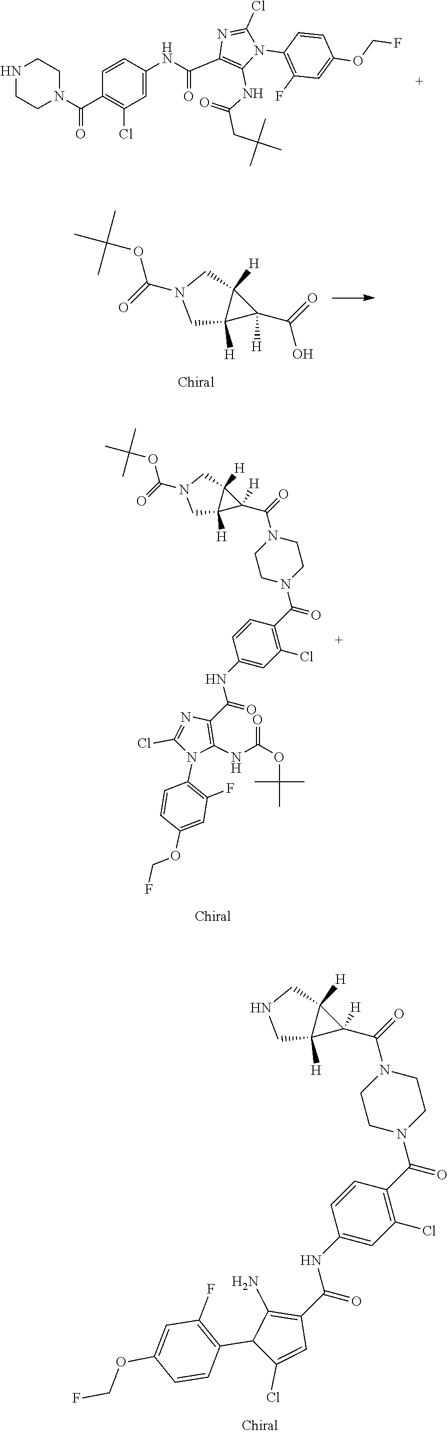 [SMILES: CC(C)(C)CC(=O)Nc1c(C(=O)Nc2ccc(C(=O)N3CCNCC3)c(Cl)c2)nc(Cl)n1-c1ccc(OCF)cc1F ; CC(C)(C)OC(=O)N1C[C@@H]2[C@H](C1)[C@H]2C(=O)O ; CC(C)(C)OC(=O)Nc1c(C(=O)Nc2ccc(C(=O)N3CCN(C(=O)[C@H]4[C@@H]5CN(C(=O)OC(C)(C)C)C[C@@H]54)CC3)c(Cl)c2)nc(Cl)n1-c1ccc(OCF)cc1F ; NC1=C(C(=O)Nc2ccc(C(=O)N3CCN(C(=O)[C@H]4[C@@H]5CNC[C@@H]54)CC3)c(Cl)c2)C=C(Cl)C1c1ccc(OCF)cc1F]

(fluoromethoxy)phenyl]imidazole-4-carboxamide 0.1:1 2,2,2-trifluoroacetic acid

To a solution of (1R,5S,6r)-3-tert-butoxycarbonyl-3-azabicyclo[3.1.0]hexane-6-carboxylic acid (12.36 mg, 52.76 μmol, 1.100 eq) and DIEA (18.6 mg, 25.1 μL, 144 μmol, 3.00 eq) in N,N-dimethylformamide (500 μL) were added HATU (20.06 mg, 52.76 μmol, 1.100 eq) and after 10 min N-[2-chloro-5-[[3-chloro-4-(piperazine-1-carbonyl)phenyl]carbamoyl]-3-[2-fluoro-4-(fluoromethoxy)phenyl]imidazol-4-yl]carbamic acid tert-butyl ester (30 mg, 48 μmol, 1.0 eq). After stirring at ambient temperature for 2 h the solvent was evaporated. The residue was dissolved in DMSO and the material purified by preparative reverse phase HPLC (column: Phenomenex Gemini-NX 5u 110A, I:100 mm, dia 30 mm) using water containing 0.05% TFA/acetonitrile as eluent to afford intermediate (22 mg) as white lyoph. solid. This solid was dissolved in dichloromethane (2 mL) and TFA (273.45 mg, 184.76 μL, 2.4 mmol, 50.000 eq) was added and the reaction stirred 4 h at ambient temperature. The reaction was diluted with water. The dichloromethane layer was washed several times with 1M HCl, until no product remained in the organic layer. The combined aqueous layers were made basic with 1 N NaOH and extracted 4 times with dichloromethane. The combined organic layers were dried over magnesium sulfate, filtered and concentrated under reduced pressure. Again, the aqueous layer were extracted several times with dichloromethane, the combined organic layers dried over magnesium sulfate, filtered and concentrated under reduced pressure, and combined with the previous crude material. This crude material was purified by preparative reverse phase HPLC (column: Phenomenex Gemini-NX 5u 110A, I:100 mm, dia 30 mm) using water containing 0.05% TFA/acetonitrile as eluent. The corresponding fractions were combined and lyophilized to afford the title compound (4.5 mg, 12% yield). MS: 632.3 [M−H]⁻, ESI neg.

### Compound 102

### 5-Amino-N-[4-[4-[(1R,5S,6r)-3-azabicyclo[3.1.0]hexane-6-carbonyl]piperazine-1-carbonyl]-3-fluoro-phenyl]-1-[2,3-difluoro-4-(fluoromethoxy)phenyl]imidazole-4-carboxamide; 1:1 2,2,2-trifluoroacetic acid

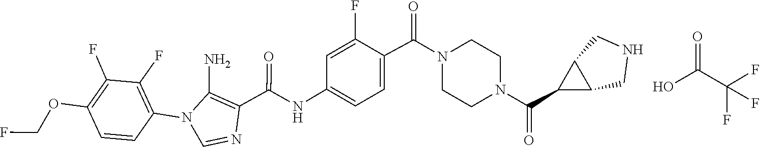

### Step 1: Ethyl 5-amino-1-(2,3-difluoro-4-methoxyphenyl)imidazole-4-carboxylate

To a solution of ethyl 2-amino-2-cyanoacetate was added 4-methylbenzene-1-sulfonic acid (8.49 g, 28.3 mmol, 3.0 eq) in triethyl orthoformate (80.0 mL, 478 mmol, 50.7 eq) and triethylamine (5.72 g, 56.6 mmol, 6.0 eq). The resulting mixture was stirred at 82° C. for 2 h, after which time the solvent was removed under reduced pressure. The residue was diluted with MeCN (40 mL), and then 2,3-difluoro-4-methoxyaniline (1.5 g, 9.4 mmol, 1.0 eq) was added and the resulting mixture was stirred at 25° C. for 15 h. The solvent was removed under reduced pressure, diluted with EtOAc (30 mL), washed with H₂O (3×25 mL), dried over Na₂SO₄ and concentrated under reduced pressure. The residue was purified by trituration with ethyl acetate to give ethyl 5-amino-1-(2,3-difluoro-4-methoxyphenyl)imidazole-4-carboxylate (900.0 mg, 3.03 mmol, 31.2% yield). MS [M+H]⁺: 298.1.

### Step 2: 5-amino-1-(2,3-difluoro-4-methoxyphenyl)imidazole-4-carboxylic acid

To a solution of ethyl 5-amino-1-(2,3-difluoro-4-methoxyphenyl)imidazole-4-carboxylate (400.0 mg, 1.35 mmol, 1.0 eq) in methanol (3 mL) were added sodium hydroxide (161.47 mg, 4.04 mmol, 3.0 eq) and water (0.5 mL) under N₂. The resulting mixture was stirred at 50° C. for 32 h. The solvent was removed under reduced pressure. The aqueous layer was then acidified with HCl (1 N) and the pH adjusted to 6-7. Filtration of the mixture yielded 5-amino-1-(2,3-difluoro-4-methoxyphenyl)imidazole-4-carboxylic acid (200.0 mg, 0.74 mmol, 55.2% yield). The crude product was used directly without further purification. MS [M+H]⁺: 270.1.

### Step 3: 5-amino-1-(2,3-difluoro-4-methoxyphenyl)imidazole-4-carbonyl chloride

A solution of 5-amino-1-(2,3-difluoro-4-methoxyphenyl)imidazole-4-carboxylic acid (150.0 mg, 0.56 mmol, 1.0 eq) in thionyl chloride (3.21 mL, 44.2 mmol, 79.4 eq) was stirred at 25° C. for 1.5 h. The resulting solution was concentrated under reduced pressure to give 5-amino-1-(2,3-difluoro-4-methoxyphenyl)imidazole-4-carbonyl chloride (150.0 mg, 0.52 mmol, 77.7% yield). MS [M−Cl+OCH₃+H]⁺: 284.0.

### Step 4: tert-butyl (1R,5S)-6-[4-[4-[[5-amino-1-(2,3-difluoro-4-methoxyphenyl)imidazole-4-carbonyl]amino]-2-fluorobenzoyl]piperazine-1-carbonyl]-3-azabicyclo[3.1.0]hexane-3-carboxylate

A solution of tert-butyl (1R,5S)-6-[4-(4-amino-2-fluorobenzoyl)piperazine-1-carbonyl]-3-azabicyclo[3.1.0]hexane-3-carboxylate (225.53 mg, 0.52 mmol, 1.0 eq) and pyridine (0.6 mL, 7.5 mmol, 14 eq) in methylene chloride (2 mL) was stirred at 0° C. Next, 5-amino-1-(2,3-difluoro-4-methoxyphenyl)imidazole-4-carbonyl chloride (150.0 mg, 0.52 mmol, 1.0 eq) in methylene chloride (2 mL) was added, and the temperature was increased to 25° C. for 1 h. The solvent was removed under reduced pressure and the residue was purified by flash column chromatography (90:10 methylene chloride:MeOH) to give tert-butyl (1R,5S)-6-[4-[4-[[5-amino-1-(2,3-difluoro-4-methoxyphenyl)imidazole-4-carbonyl]amino]-2-fluorobenzoyl]piperazine-1-carbonyl]-3-azabicyclo[3.1.0]hexane-3-carboxy-late (100.0 mg, 0.15 mmol, 27.2% yield). MS [M+H]⁺: 684.2.

### Step 5: 5-amino-N-[4-[4-[(1R,5S)-3-azabicyclo[3.1.0]hexane-6-carbonyl]piperazine-1-carbonyl]-3-fluorophenyl]-1-(2,3-difluoro-4-hydroxy-phenyl)imidazole-4-carboxamide

To a solution of tert-butyl (1R,5S)-6-[4-[4-[[5-amino-1-(2,3-difluoro-4-methoxyphenyl)imidazole-4-carbonyl]amino]-2-fluorobenzoyl]piperazine-1-carbonyl]-3-azabicyclo[3.1.0]hexane-3-carboxylate (80.0 mg, 0.12 mmol, 1.0 eq) in methylene chloride (2 mL) was added boron tribromide (293.14 mg, 1.17 mmol, 10.0 eq) dropwise at 0° C. under N₂ atmosphere. The resulting mixture was allowed to warm to 25° C. and stirred for 16 h. Filtration of the reaction mixture afforded crude 5-amino-N-[4-[4-[(1R,5S)-3-azabicyclo[3.1.0]hexane-6-carbonyl]piperazine-1-carbonyl]-3-fluorophenyl]-1-(2,3-difluoro-4-hydroxyphenyl)imidazole-4-carboxamide (60.0 mg, 0.11 mmol, 82.8% yield), which was used directly without further purification. MS [M+H]⁺: 570.1.

### Step 6: tert-butyl (1S,5R)-6-[4-[4-[[5-amino-1-(2,3-difluoro-4-hydroxyphenyl)imidazole-4-carbonyl]amino]-2-fluorobenzoyl]piperazine-1-carbonyl]-3-azabicyclo[3.1.0]hexane-3-carboxylate

To a solution of 5-amino-N-[4-[4-[(1S,5R)-3-azabicyclo[3.1.0]hexane-6-carbonyl]piperazine-1-carbonyl]-3-fluoro-phenyl]-1-(2,3-difluoro-4-hydroxyphenyl)imidazole-4-carboxamide (60.0 mg, 0.11 mmol, 1.0 eq) in THF (2 mL) and H₂O (2 mL) were added sodium bicarbonate (17.7 mg, 0.21 mmol, 2.0 eq) and di-tert-butyl dicarbonate (22.99 mg, 0.11 mmol, 1.0 eq) under N₂ atmosphere. The vessel was sealed and the mixture was stirred at 25° C. for 3 h. Concentration under reduced pressure and purification by flash column chromatography on silica gel (90:10 methylene chloride:MeOH) yielded tert-butyl (1S,5R)-6-[4-[4-[[5-amino-1-(2,3-difluoro-4-hydroxy-phenyl)imidazole-4-carbonyl]amino]-2-fluorobenzoyl]piperazine-1-carbonyl]-3-azabicyclo[3.1.0]hexane-3-carboxylate (50.0 mg, 0.07 mmol, 63.1% yield). MS [M+H]⁺: 670.0.

### Step 7: tert-butyl (1R,5S)-6-[4-[4-[[5-amino-1-[2,3-difluoro-4-(fluoromethoxy)phenyl]imidazole-4-carbonyl]amino]-2-fluorobenzoyl]piperazine-1-carbonyl]-3-azabicyclo[3.1.0]hexane-3-carboxylate

To a solution of tert-butyl (1R,5S)-6-[4-[4-[[5-amino-1-(2,3-difluoro-4-hydroxyphenyl)imidazole-4-carbonyl]amino]-2-fluorobenzoyl]piperazine-1-carbonyl]-3-azabicyclo[3.1.0]hexane-3-carboxylate (40.0 mg, 0.06 mmol, 1.0 eq) in MeCN (2 mL) were added potassium carbonate (24.77 mg, 0.18 mmol, 3.0 eq) and fluoro(iodo)methane (14.33 mg, 0.09 mmol, 1.5 eq) under N₂ atmosphere. The vessel was sealed and the mixture was stirred at 50° C. for 18 h. Concentration under reduced pressure and purification by flash column chromatography on silica gel (85:15 methylene chloride:MeOH) afforded tert-butyl (1R,5S)-6-[4-[4-[[5-amino-1-[2,3-difluoro-4-(fluoromethoxy)phenyl]imidazole-4-carbonyl]amino]-2-fluorobenzoyl]piperazine-1-carbonyl]-3-azabicyclo[3.1.0]hexane-3-carboxylate (42.0 mg, 0.06 mmol, 98.2% yield). MS [M−Boc+H]⁺: 646.1.

### Step 8: 5-amino-N-[4-[4-[(1R,5S)-3-azabicyclo[3.1.0]hexane-6-carbonyl]piperazine-1-carbonyl]-3-fluorophenyl]-1-[2,3-difluoro-4-(fluoromethoxy)phenyl]imidazole-4-carboxamide; 1:1 2,2,2-trifluoroacetic acid

To a solution of tert-butyl (1R,5S)-6-[4-[4-[[5-amino-1-[2,3-difluoro-4-(fluoromethoxy)phenyl]imidazole-4-carbonyl]amino]-2-fluorobenzoyl]piperazine-1-carbonyl]-3-azabicyclo[3.1.0]hexane-3-carboxylate (30.0 mg, 0.04 mmol, 1.0 eq) in methylene chloride (2 mL) was added trifluoroacetic acid (0.6 mL, 8.1 mmol, 190 eq). The resulting mixture was stirred at 25° C. for 2 h. The solvent was then removed under reduced pressure and the residue was purified by prep-HPLC [Gemini-C18, 150×21.2 mm, 5 μm, MeCN-H₂O (0.1% TFA), 15%˜40%] to provide 5-amino-N-[4-[4-[(1R,5S)-3-azabicyclo[3.1.0]hexane-6-carbonyl]piperazine-1-carbonyl]-3-fluorophenyl]-1-[2,3-difluoro-4-(fluoromethoxy)phenyl]imidazole-4-carboxamide; 2,2,2-trifluoroacetic acid (11.5 mg, 0.02 mmol, 44.3% yield). MS [M+H]⁺: 602.3.

### Compound 103

### 5-Amino-N-[4-[4-[(1R,5S,6r)-3-azabicyclo[3.1.0]hexane-6-carbonyl]piperazine-1-carbonyl]-3-fluorophenyl]-1-[2-chloro-3-fluoro-4-(fluoromethoxy)phenyl]imidazole-4-carboxamide; 1:1 formic acid

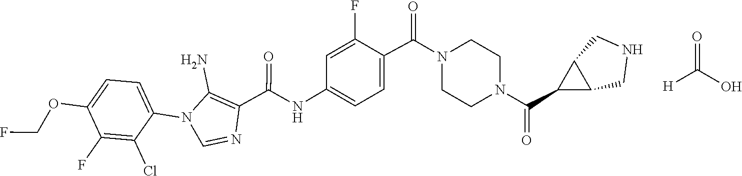

### Step 1: 3-chloro-2-fluoro-1-(fluoromethoxy)-4-nitrobenzene

To a solution of 3-chloro-2-fluoro-4-nitrophenol (5.0 g, 26.1 mmol, 1.0 eq) in MeCN (50 mL) were added N,N-diisopropylethylamine (10.12 g, 78.31 mmol, 3.0 eq) and fluoro(iodo)methane (5.01 g, 31.3 mmol, 1.2 eq) under N₂ atmosphere. The vessel was sealed, and the mixture was stirred at 45° C. for 15 h. Concentration under reduced pressure and purification by flash column chromatography on silica gel (1:1 petroleum ether:EtOAc) yielded 3-chloro-2-fluoro-1-(fluoromethoxy)-4-nitrobenzene (4.8 g, 21 mmol, 81% yield). MS [M−H]⁻: 222.0.

### Step 2: 2-chloro-3-fluoro-4-(fluoromethoxy)aniline

To a solution of 3-chloro-2-fluoro-1-(fluoromethoxy)-4-nitrobenzene (4.8 g, 21 mmol, 1.0 eq) in ethanol (50 mL) and water (10 mL) were added ammonium chloride (5.74 g, 107 mmol, 5.0 eq) and iron powder (5.99 g, 107 mmol, 5.0 eq) under N₂ atmosphere. The resulting mixture was stirred at 45° C. After 5 h, the reaction mixture was filtered, washed with methanol, and concentrated under reduced pressure. The residue was then diluted with EtOAc (10 mL), washed with H₂O (3×10 mL), dried over Na₂SO₄ and concentrated under reduced pressure. The residue was purified by flash column chromatography (70:30 petroleum ether:EtOAc) to afford 2-chloro-3-fluoro-4-(fluoromethoxy)aniline (3.4 g, 18 mmol, 82% yield). MS [M+H]⁺: 193.9.

### Step 3: ethyl 5-amino-1-[2-chloro-3-fluoro-4-(fluoromethoxy)phenyl]imidazole-4-carboxylate

A solution of ethyl 2-amino-2-cyanoacetate; 4-methylbenzene-1-sulfonic acid (620.58 mg, 2.07 mmol, 2.0 eq), triethylamine (418.18 mg, 4.13 mmol, 4.0 eq) and triethyl orthoformate (765.57 mg, 5.17 mmol, 5.0 eq) in MeCN (3 mL) was heated at reflux for 2 h. After cooling to room temperature, 2-chloro-3-fluoro-4-(fluoromethoxy)aniline (200.0 mg, 1.03 mmol, 1.0 eq) was added. The mixture was stirred for 18 h at room temperature. The solvent was removed under reduced pressure, then diluted with EtOAc (3×20 mL), washed with H₂O (20 mL), dried over Na₂SO₄ and concentrated under reduced pressure. The residue was purified by flash column chromatography (90:10 methylene chloride:MeOH) to afford ethyl 5-amino-1-[2-chloro-3-fluoro-4-(fluoromethoxy)phenyl]imidazole-4-carboxylate (40.0 mg, 0.12 mmol, 9.2% yield). MS [M+H]⁺: 331.8.

### Step 4: 5-amino-1-[2-chloro-3-fluoro-4-(fluoromethoxy)phenyl]imidazole-4-carboxylic acid

To a solution of ethyl 5-amino-1-[2-chloro-3-fluoro-4-(fluoromethoxy)phenyl]imidazole-4-carboxylate (150.0 mg, 0.45 mmol, 1.0 eq) in 1:1 ethanol:water (2 mL) was added sodium hydroxide (36.18 mg, 0.9 mmol, 2.0 eq) under N₂. The resulting mixture was stirred at 80° C. for 4 h and then cooled to room temperature. After adjustment of the pH to 7-8, the mixture was filtered to afford 5-amino-1-[2-chloro-3-fluoro-4-(fluoromethoxy)phenyl]imidazole-4-carboxylic acid (120.0 mg, 0.4 mmol, 85.6% yield). MS [M+H]⁺: 303.8.

### Step 5: 5-amino-1-[2-chloro-3-fluoro-4-(fluoromethoxy)phenyl]imidazole-4-carbonyl chloride

To a solution of 5-amino-1-[2-chloro-3-fluoro-4-(fluoromethoxy)phenyl]imidazole-4-carboxylic acid (120.0 mg, 0.28 mmol, 1.0 eq) was added thionyl chloride (3.36 mL, 46.3 mmol, 167 eq) under N₂ atmosphere. The mixture was stirred at 0° C. for 1 h. The solvent was removed under reduced pressure to afford 5-amino-1-[2-chloro-3-fluoro-4-(fluoromethoxy)phenyl]imidazole-4-carbonyl chloride (89.0 mg, 0.28 mmol, 97.9% yield). MS [M−Cl+OCH₃+H]⁺: 317.8.

### Step 6: tert-Butyl (1R,5S)-6-[4-[4-[[5-amino-1-[2-chloro-3-fluoro-4-(fluoromethoxy)phenyl]imidazole-4-carbonyl]amino]-2-fluorobenzoyl]piperazine-1-carbonyl]-3-azabicyclo[3.1.0]hexane-3-carboxylate

A solution of tert-butyl (1R,5S)-6-[4-(4-amino-2-fluorobenzoyl)piperazine-1-carbonyl]-3-azabicyclo[3.1.0]hexane-3-carboxylate (67.14 mg, 0.16 mmol, 1.0 eq) and pyridine (36.84 mg, 0.47 mmol, 3.0 eq) in methylene chloride (2 mL) was stirred at 0° C. Then tert-butyl (1R,5S)-6-[4-(4-amino-2-fluorobenzoyl)piperazine-1-carbonyl]-3-azabicyclo[3.1.0]hexane-3-carboxylate (67.14 mg, 0.16 mmol, 1.0 eq) was added under N₂ atmosphere. The mixture was stirred at 0° C. for 1 h. The solvent was removed under reduced pressure and the residue was purified by flash column chromatography (90:10 methylene chloride:MeOH) to afford tert-butyl (1R,5S)-6-[4-[4-[[5-amino-1-[2-chloro-3-fluoro-4-(fluoromethoxy)phenyl]imidazole-4-carbonyl]amino]-2-fluorobenzoyl]piperazine-1-carbonyl]-3-azabicyclo[3.1.0]hexane-3-carboxylate (30.0 mg, 0.04 mmol, 16.7% yield). MS [M−tBu+H]⁺: 661.6.

### Step 7: 5-amino-N-[4-[4-[(1R,5S)-3-azabicyclo[3.1.0]hexane-6-carbonyl]piperazine-1-carbonyl]-3-fluorophenyl]-1-[2-chloro-3-fluoro-4-(fluoromethoxy)phenyl]imidazole-4-carboxamide; 1:1 formic acid

To a solution of tert-butyl (1R,5S)-6-[4-[4-[[5-amino-1-[2-chloro-3-fluoro-4-(fluoromethoxy)phenyl]imidazole-4-carbonyl]amino]-2-fluorobenzoyl]piperazine-1-carbonyl]-3-azabicyclo[3.1.0]hexane-3-carboxylate (20.0 mg, 0.03 mmol, 1.0 eq) in methylene chloride (2 mL) was added trifluoroacetic acid (31.76 mg, 0.28 mmol, 10.0 eq). The resulting mixture was stirred at 25° C. for 1 h. The solvent was removed under reduced pressure and the residue was purified by prep-HPLC [Gemini-C18, 150×21.2 mm, 5 μm, MeCN-H₂O (0.1% FA), 15%˜40%] to afford 5-amino-N-[4-[4-[(1R,5S)-3-azabicyclo[3.1.0]hexane-6-carbonyl]piperazine-1-carbonyl]-3-fluorophenyl]-1-[2-chloro-3-fluoro-4-(fluoromethoxy)phenyl]imidazole-4-carboxamide; 1:1 formic acid (14.6 mg, 0.02 mmol, 83.4% yield). MS [M+H]⁺: 618.2.

### Compound 104

### 5-amino-N-[4-[4-[(1S,5R,6r)-3-azabicyclo[3.1.0]hexane-6-carbonyl]piperazine-1-carbonyl]-3-(fluoromethyl)phenyl]-2-chloro-1-[2,3-difluoro-4-(fluoromethoxy)phenyl]imidazole-4-carboxamide; 1:1 2,2,2-trifluoroacetic acid

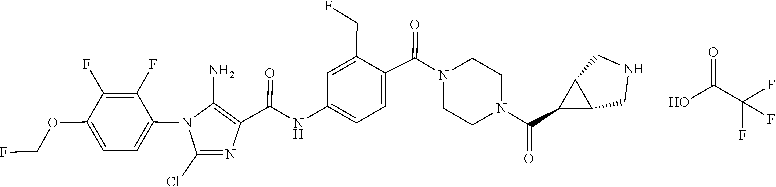 [SMILES: Nc1c(C(=O)Nc2ccc(C(=O)N3CCN(C(=O)[C@H]4[C@@H]5CNC[C@@H]54)CC3)c(CF)c2)nc(Cl)n1-c1ccc(OCF)c(F)c1F ; O=C(O)C(F)(F)F]

### Step 1: tert-butyl 4-[2-(hydroxymethyl)-4-nitrobenzoyl]piperazine-1-carboxylate

Trimethylaluminum (2.0 M in toluene)(6.36 mL, 12.7 mmol, 2.0 eq) was slowly added to a solution of 1-BOC-piperazine (2370.62 mg, 12.73 mmol, 2.0 eq) in methylene chloride (30 mL) under nitrogen at room temperature. The mixture was stirred at room temperature for 15 min, and then 5-nitro-3H-isobenzofuran-1-one (1140.0 mg, 6.36 mmol, 1.0 eq) was added. The mixture was stirred at 25° C. under nitrogen for 3 h. The reaction was carefully quenched with dilute HCl and extracted with methylene chloride. The organic extract was dried (MgSO₄) and concentrated. The residue was then purified by flash chromatography with 3:1 petroleum ether:EtOAc elution to afford tert-butyl 4-[2-(hydroxymethyl)-4-nitrobenzoyl]piperazine-1-carboxylate (1300.0 mg, 3.56 mmol, 52% yield). MS [M+Na]⁺: 388.1.

### Step 2: tert-Butyl 4-[2-(fluoromethyl)-4-nitrobenzoyl]piperazine-1-carboxylate

To a solution of tert-butyl 4-[2-(hydroxymethyl)-4-nitrobenzoyl]piperazine-1-carboxylate (1250.0 mg, 3.42 mmol, 1.0 eq) in methylene chloride (20 mL) was added (diethylamino) sulfur trifluoride (727.9 mg, 4.52 mmol, 1.32 eq) under N₂ atmosphere. The resulting mixture was stirred at 0° C. for 2 h, diluted with EtOAc (100 mL), and washed with H₂O (3×50 mL). After drying over Na₂SO₄ and concentrating under reduced pressure, the residue was purified by flash column chromatography (65:35 petroleum ether:EtOAc elution) to give tert-butyl 4-[2-(fluoromethyl)-4-nitrobenzoyl]piperazine-1-carboxylate (750.0 mg, 2.04 mmol, 59.6% yield). MS [M+Na]⁺: 390.1.

### Step 3: [2-(fluoromethyl)-4-nitrophenyl]-piperazin-1-yl-methanone

To a solution of tert-butyl 4-[2-(fluoromethyl)-4-nitrobenzoyl]piperazine-1-carboxylate (740.0 mg, 2.01 mmol, 1.0 eq) in methylene chloride (10 mL) was added trifluoroacetic acid (0.78 mL, 10.07 mmol, 5.0 eq) under N₂ atmosphere. The resulting mixture was stirred at 25° C. for 1 h. The solvent was removed under reduced pressure to give [2-(fluoromethyl)-4-nitrophenyl]-piperazin-1-yl-methanone (520.0 mg, 1.95 mmol, 93% yield). MS [M+H]⁺: 268.1.

### Step 4: tert-butyl (1S,5R)-6-[4-[2-(fluoromethyl)-4-nitrobenzoyl]piperazine-1-carbonyl]-3-azabicyclo[3.1.0]hexane-3-carboxylate

To a solution of [2-(fluoromethyl)-4-nitro-phenyl]-piperazin-1-yl-methanone (520.0 mg, 1.95 mmol, 1.0 eq) in methylene chloride (20 mL) were added (1S,5R)-3-tert-butoxycarbonyl-3-azabicyclo[3.1.0]hexane-6-carboxylic acid (663.26 mg, 2.92 mmol, 1.5 eq), 2-(7-aza-1H-benzotriazole-1-yl)-1,1,3,3-tetramethyluronium hexafluorophosphate (1479.6 mg, 3.89 mmol, 2.0 eq) and N, N-diisopropylethylamine (1005.83 mg, 7.78 mmol, 4.0 eq) under N₂ atmosphere. The resulting mixture was stirred at 25° C. for 1 h. After dilution with methylene chloride (50 mL), the mixture was washed with H₂O (3×25 mL), dried over Na₂SO₄ and concentrated under reduced pressure. The residue was purified by flash column chromatography (97:3 methylene chloride:MeOH elution) to give tert-butyl (1S,5R)-6-[4-[2-(fluoromethyl)-4-nitrobenzoyl]piperazine-1-carbonyl]-3-azabicyclo[3.1.0]hexane-3-carboxylate (875.0 mg, 1.84 mmol, 94% yield). MS [M+Na]⁺: 499.1.

### Step 5: tert-butyl (1S,5R)-6-[4-[4-amino-2-(fluoromethyl)benzoyl]piperazine-1-carbonyl]-3-azabicyclo[3.1.0]hexane-3-carboxylate

To a solution of tert-butyl (1S,5R)-6-[4-[2-(fluoromethyl)-4-nitrobenzoyl]piperazine-1-carbonyl]-3-azabicyclo[3.1.0]hexane-3-carboxylate (825.0 mg, 1.73 mmol, 1.0 eq) in ethanol (15 mL) were added iron powder (483.44 mg, 8.66 mmol, 5.0 eq), ammonium chloride (926.11 mg, 17.31 mmol, 10.0 eq) and water (3.0 mL) under N₂ atmosphere. The resulting mixture was stirred at 50° C. for 3 h. After filtration, the filtrate was diluted with EtOAc (50 mL), washed with H₂O (3×25 mL), dried over Na₂SO₄ and concentrated under reduced pressure. The residue was purified by flash column chromatography (95:5 methylene chloride:MeOH elution) to give tert-butyl (1S,5R)-6-[4-[4-amino-2-(fluoromethyl)benzoyl]piperazine-1-carbonyl]-3-azabicyclo[3.1.0]hexane-3-carboxylate (660.0 mg, 1.48 mmol, 82% yield). MS [M+Na]⁺: 469.2.

### Step 6: ethyl 5-amino-2-chloro-1-[2,3-difluoro-4-(fluoromethoxy)phenyl]imidazole-4-carboxylate

To a solution of ethyl 5-amino-1-[2,3-difluoro-4-(fluoromethoxy)phenyl]imidazole-4-carboxylate (350.0 mg, 1.11 mmol, 1.0 eq) in DMF (6.0 mL) was added N-chlorosuccinimide (192.72 mg, 1.44 mmol, 1.3 eq) under N₂ atmosphere. The resulting mixture was stirred at 50° C. for 3 h. The reaction mixture was diluted with EtOAc (30 mL), washed with H₂O (3×15 mL), dried over Na₂SO₄ and concentrated under reduced pressure. The residue was purified by flash column chromatography (65:35 petroleum ether:EtOAc elution) to give ethyl 5-amino-2-chloro-1-[2,3-difluoro-4-(fluoromethoxy)phenyl]imidazole-4-carboxylate (220.0 mg, 0.63 mmol, 53.3% yield). MS [M+H]⁺: 350.0.

### Step 7: 5-amino-2-chloro-1-[2,3-difluoro-4-(fluoromethoxy)phenyl]imidazole-4-carboxylic acid

To a solution of ethyl 5-amino-2-chloro-1-[2,3-difluoro-4-(fluoromethoxy)phenyl]imidazole-4-carboxylate (170.0 mg, 0.49 mmol, 1.0 eq) in ethanol (25 mL) were added sodium hydroxide (77.78 mg, 1.94 mmol, 4.0 eq) and water (8.5 mL, 470 mmol, 970 eq) under N₂. The resulting mixture was stirred at 50° C. for 72 h. The solvent was then removed under reduced pressure. The residue was diluted with H₂O (30 mL) and extracted with EtOAc (3×15 mL). The aqueous layer was acidified with HCl (4 N) and filtered to give 5-amino-2-chloro-1-[2,3-difluoro-4-(fluoromethoxy)phenyl]imidazole-4-carboxylic acid (60.0 mg, 0.19 mmol, 26.8% yield), the crude product was used directly without further purification. MS [M+H]⁺: 322.0.

### Step 8: 5-amino-2-chloro-1-[2,3-difluoro-4-(fluoromethoxy)phenyl]imidazole-4-carbonyl chloride

5-amino-2-chloro-1-[2,3-difluoro-4-(fluoromethoxy)phenyl]imidazole-4-carboxylic acid (60.0 mg, 0.19 mmol, 1.0 eq) was dissolved in thionyl chloride (2.0 mL, 28 mmol, 150 eq) at 0° C. under N₂ atmosphere. The resulting mixture was stirred at 25° C. for 1 h. The solvent was removed under reduced pressure to give 5-amino-2-chloro-1-[2,3-difluoro-4-(fluoromethoxy)phenyl]imidazole-4-carbonyl chloride (65.0 mg, 0.19 mmol, 73.8% yield). MS [M−Cl+OCH₃+H]⁺: 336.0.

### Step 9: tert-butyl (1S,5R)-6-[4-[4-[[5-amino-2-chloro-1-[2,3-difluoro-4-(fluoromethoxy)phenyl]imidazole-4-carbonyl]amino]-2-(fluoromethyl)benzoyl]piperazine-1-carbonyl]-3-azabicyclo[3.1.0]hexane-3-carboxylate

To a solution of tert-butyl (1S,5R)-6-[4-[4-amino-2-(fluoromethyl)benzoyl]piperazine-1-carbonyl]-3-azabicyclo[3.1.0]hexane-3-carboxylate (43.01 mg, 0.1 mmol, 0.7 eq) in methylene chloride (2 mL) were added pyridine (0.22 mL, 2.75 mmol, 20.0 eq) and 5-amino-2-chloro-1-[2,3-difluoro-4-(fluoromethoxy)phenyl]imidazole-4-carbonyl chloride (65.0 mg, 0.14 mmol, 1.0 eq) at 0° C. under N₂ atmosphere. The resulting mixture was then allowed to warm to 25° C. and stirred for 0.5 h. The solvent was removed under reduced pressure, diluted with EtOAc (25 mL), washed with H₂O (3×15 mL), dried over Na₂SO₄ and concentrated under reduced pressure. The resulting residue was purified by flash column chromatography (96:4 methylene chloride:MeOH) to give tert-butyl (1S,5R)-6-[4-[4-[[5-amino-2-chloro-1-[2,3-difluoro-4-(fluoromethoxy)phenyl]imidazole-4-carbonyl]amino]-2-(fluoromethyl)benzoyl]piperazine-1-carbonyl]-3-azabicyclo[3.1.0]hexane-3-carboxylate (80.0 mg, 0.11 mmol, 38.8% yield). MS [M+Na]⁺: 772.1.

### Step 10: 5-amino-N-[4-[4-[(1S,5R)-3-azabicyclo[3.1.0]hexane-6-carbonyl]piperazine-1-carbonyl]-3-(fluoromethyl)phenyl]-2-chloro-1-[2,3-difluoro-4-(fluoromethoxy)phenyl]imidazole-4-carboxamide;2,2,2-trifluoroacetic acid

To a solution of tert-butyl (1S,5R)-6-[4-[4-[[5-amino-2-chloro-1-[2,3-difluoro-4-(fluoromethoxy)phenyl]imidazole-4-carbonyl]amino]-2-(fluoromethyl)benzoyl]piperazine-1-carbonyl]-3-azabicyclo[3.1.0]hexane-3-carboxylate (80.0 mg, 0.05 mmol, 1.0 eq) in methanol (0.500 mL) was added trifluoroacetic acid (1.0 mL, 13 mmol, 240 eq). The resulting mixture was stirred at 25° C. for 0.5 h. After solvent removal under reduced pressure, the residue was purified by prep-HPLC [Gemini-C18, 150×21.2 mm, 5 μm, MeCN-H₂O (0.1% TFA), 30%˜80%] to give 5-amino-N-[4-[4-[(1S,5R)-3-azabicyclo[3.1.0]hexane-6-carbonyl]piperazine-1-carbonyl]-3-(fluoromethyl)phenyl]-2-chloro-1-[2,3-difluoro-4-(fluoromethoxy)phenyl]imidazole-4-carboxamide;2,2,2-trifluoroacetic acid (14.3 mg, 0.02 mmol, 34.8% yield). MS [M+H]⁺: 650.1.

### Compound 105

### 5-amino-N-[4-[4-[(1R,5S,6r)-3-azabicyclo[3.1.0]hexane-6-carbonyl]piperazine-1-carbonyl]-3-chlorophenyl]-1-[2-chloro-3-fluoro-4-(fluoromethoxy)phenyl]imidazole-4-carboxamide; 1:1 2,2,2-trifluoroacetic acid

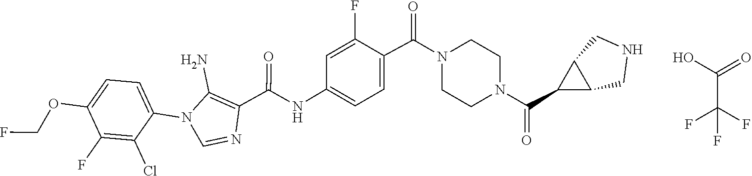 [SMILES: Nc1c(C(=O)Nc2ccc(C(=O)N3CCN(C(=O)[C@H]4[C@@H]5CNC[C@@H]54)CC3)c(F)c2)ncn1-c1ccc(OCF)c(F)c1Cl ; O=C(O)C(F)(F)F]

### Step 1: tert-butyl 4-(2-chloro-4-nitrobenzoyl)piperazine-1-carboxylate

To a solution of 2-chloro-4-nitrobenzoic acid (5.0 g, 25 mmol, 1.0 eq) in methylene chloride (100 mL) were added 1-BOC-piperazine (4.85 g, 26.0 mmol, 1.05 eq), propylphosphonic anhydride (23.68 g, 37.21 mmol, 1.5 eq) and N,N-diisopropylethylamine (6.41 g, 49.6 mmol, 2.0 eq). The resulting mixture was stirred at 25° C. for 2 h. After solvent removal under reduced pressure, the residue was diluted with EtOAc (100 mL), washed with H₂O (4×60 mL), dried over Na₂SO₄ and concentrated under reduced pressure to afford tert-butyl 4-(2-chloro-4-nitrobenzoyl)piperazine-1-carboxylate (7.20 g, 19.5 mmol, 47.1% yield). MS [(M+Na)+H]⁺: 392.1.

### Step 2: (2-chloro-4-nitrophenyl)-piperazin-1-yl-methanone

To a solution of tert-butyl 4-(2-chloro-4-nitrobenzoyl)piperazine-1-carboxylate (7.2 g, 19 mmol, 1.0 eq) and hydrogen chloride-1,4-dioxane solution (15.0 mL) in methylene chloride (45 mL) was stirred at 25° C. for 2 h. The combined organic layer was dried over anhydrous sodium sulfate and concentrated under reduced pressure to give (2-chloro-4-nitrophenyl)-piperazin-1-yl-methanone (5.0 g, 18.5 mmol, 90% yield). MS [M+H]⁺: 270.0

### Step 3: tert-butyl (1S,5R)-6-[4-(2-chloro-4-nitrobenzoyl)piperazine-1-carbonyl]-3-azabicyclo[3.1.0]hexane-3-carboxylate

To a solution of (2-chloro-4-nitrophenyl)-piperazin-1-yl-methanone (3.0 g, 11 mmol, 1.0 eq), (1S,5R)-3-tert-butoxycarbonyl-3-azabicyclo[3.1.0]hexane-6-carboxylic acid (2.53 g, 11.1 mmol, 1.0 eq), propylphosphonic anhydride (17.7 g, 55.6 mmol, 5.0 eq) and N,N-diisopropylethylamine (4.31 g, 33.4 mmol, 3.0 eq). The resulting mixture was stirred at 25° C. for 3 h. After solvent removal under reduced pressure, the residue was diluted with methylene chloride (100 mL), washed with H₂O (4×60 mL), dried over Na₂SO₄ and concentrated under reduced pressure to give tert-butyl (1S,5R)-6-[4-(2-chloro-4-nitrobenzoyl)piperazine-1-carbonyl]-3-azabicyclo[3.1.0]hexane-3-carboxylate (4.3 g, 9.0 mmol, 80% yield). MS [(M−C₄H₈)+H]⁺: 423.0.

### Step 4: tert-butyl (1S,5R)-6-[4-(4-amino-2-chlorobenzoyl)piperazine-1-carbonyl]-3-azabicyclo[3.1.0]hexane-3-carboxylate

A solution of tert-butyl (1S,5R)-6-[4-(2-chloro-4-nitrobenzoyl)piperazine-1-carbonyl]-3-azabicyclo[3.1.0]hexane-3-carboxylate (4.3 g, 9.0 mmol, 1.0 eq), iron powder (2.51 g, 44.9 mmol, 5.0 eq) and ammonium chloride (2.4 g, 45 mmol, 5.0 eq) in ethanol (40 mL) and water (8 mL) was stirred at 50° C. for 6 h. The solid was removed by filtration, and the reaction mixture was partitioned between methylene chloride and water. The aqueous layer was extracted again with methylene chloride. The combined organic layer was dried over anhydrous sodium sulfate and concentrated under reduced pressure to give the tert-butyl (1S,5R)-6-[4-(4-amino-2-chlorobenzoyl)piperazine-1-carbonyl]-3-azabicyclo[3.1.0]hexane-3-carboxylate (3.2 g, 7.1 mmol, 78% yield). MS [(M−C₄H₈)+H]⁺: 393.1.

### Step 5: 3-chloro-2-fluoro-1-(fluoromethoxy)-4-nitrobenzene

To a solution of 3-chloro-2-fluoro-4-nitrophenol (5.0 g, 26 mmol, 1.0 eq) in MeCN (50 mL) were added N,N-diisopropylethylamine (10.12 g, 78.31 mmol, 3.0 eq) and fluoro(iodo)methane (5.01 g, 31.3 mmol, 1.2 eq) under N₂ atmosphere. The vessel was sealed, and the mixture was stirred at 45° C. for 15 h. After concentration under reduced pressure, the residue was purified by flash column chromatography on silica gel (1:1 petroleum ether:EtOAc) to afford 3-chloro-2-fluoro-1-(fluoromethoxy)-4-nitrobenzene (4.8 g, 21 mmol, 81% yield).

### Step 6: ethyl 5-amino-1-[2-chloro-3-fluoro-4-(fluoromethoxy)phenyl]imidazole-4-carboxylate

A solution of ethyl 2-amino-2-cyanoacetate; 4-methylbenzene-1-sulfonic acid (620.58 mg, 2.07 mmol, 2.0 eq), triethylamine (418.18 mg, 4.13 mmol, 4.0 eq) and triethyl orthoformate (765.57 mg, 5.17 mmol, 5.0 eq) in MeCN (3 mL) was heated at reflux for 2 h. After cooling down to room temperature, 2-chloro-3-fluoro-4-(fluoromethoxy)aniline (200.0 mg, 1.03 mmol, 1.0 eq) was added. The mixture was stirred for 18 h at room temperature. The solvent was removed under reduced pressure, then diluted with EtOAc (3×20 mL), washed with H₂O (20 mL), dried over Na₂SO₄ and concentrated under reduced pressure. The residue was purified by flash column chromatography (90:10 methylene chloride:MeOH) to give ethyl 5-amino-1-[2-chloro-3-fluoro-4-(fluoromethoxy)phenyl]imidazole-4-carboxylate (40.0 mg, 0.12 mmol, 9.2% yield). MS [M+H]⁺: 331.8.

### Step 7: 5-amino-1-[2-chloro-3-fluoro-4-(fluoromethoxy)phenyl]imidazole-4-carboxylic acid

To a solution of ethyl 5-amino-1-[2-chloro-3-fluoro-4-(fluoromethoxy)phenyl]imidazole-4-carboxylate (150.0 mg, 0.45 mmol, 1.0 eq) in ethanol (1 mL) were added potassium hydroxide (36.18 mg, 0.9 mmol, 2.0 eq) and water (1 mL) under N₂. The resulting mixture was stirred at 80° C. for 4 h. The reaction solution was cooled to room temperature and the pH of the reaction solution adjusted to 7-8. Filtration of the mixture yielded 5-amino-1-[2-chloro-3-fluoro-4-(fluoromethoxy)phenyl]imidazole-4-carboxylic acid (120.0 mg, 0.4 mmol, 85.6% yield). MS [M+H]⁺: 303.8.

### Step 8: 5-amino-1-[2-chloro-3-fluoro-4-(fluoromethoxy)phenyl]imidazole-4-carbonyl chloride

To a solution of 5-amino-1-[2-chloro-3-fluoro-4-(fluoromethoxy)phenyl]imidazole-4-carboxylic acid (120.0 mg, 0.28 mmol, 1.0 eq) in thionyl chloride (3.36 mL, 46.3 mmol, 167.23 eq) under N₂ atmosphere. The mixture was stirred at 0° C. for 1 h. The solvent was removed under reduced pressure to give 5-amino-1-[2-chloro-3-fluoro-4-(fluoromethoxy)phenyl]imidazole-4-carbonyl chloride (89.0 mg, 0.28 mmol, 97.9% yield). MS [(M−Cl+OCH₃)+H]⁺: 317.8.

### Step 9: tert-butyl (1R,5S)-6-[4-[4-[[5-amino-1-[2-chloro-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate

A solution of tert-butyl (1R,5S)-6-[4-(4-amino-2-chlorobenzoyl)piperazine-1-carbonyl]-3-azabicyclo[3.1.0]hexane-3-carboxylate (69.69 mg, 0.16 mmol, 1.0 eq) and pyridine (36.84 mg, 0.47 mmol, 3.0 eq) in methylene chloride (2 mL) was stirred at 0° C. Then 5-amino-1-[2-chloro-3-fluoro-4-(fluoromethoxy)phenyl]imidazole-4-carbonyl chloride (50.0 mg, 0.16 mmol, 1.0 eq) was added under N₂ atmosphere. The mixture was stirred at 0° C. for 1 h. After concentration under reduced pressure, the residue was purified by flash column chromatography on silica gel (90:10 methylene chloride:MeOH) to give tert-butyl (1R,5S)-6-[4-[4-[[5-amino-1-[2-chloro-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (40.0 mg, 0.05 mmol, 34.4% yield). MS [(M−C₄H₈)+H]⁺: 677.7.

### Step 10: 5-amino-N-[4-[4-[(1R,5S)-3-azabicyclo[3.1.0]hexane-6-carbonyl]piperazine-1-carbonyl]-3-chlorophenyl]-1-[2-chloro-3-fluoro-4-(fluoromethoxy)phenyl]imidazole-4-carboxamide; 1:1 2,2,2-trifluoroacetic acid

To a solution of tert-butyl (1R,5S)-6-[4-[4-[[5-amino-1-[2-chloro-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (30.0 mg, 0.04 mmol, 1.0 eq) in methylene chloride (2 mL) was added trifluoroacetic acid (0.25 mL, 3.4 mmol, 82 eq) under N₂ atmosphere. The resulting mixture was stirred at 25° C. for 2 h. Afterwards the solvent was removed under reduced pressure and the residue was purified by prep-HPLC [Gemini-C18, 150×21.2 mm, 5 μm, MeCN-H₂O (0.1% FA), 15%˜40%] to give 5-amino-N-[4-[4-[(1R,5S)-3-azabicyclo[3.1.0]hexane-6-carbonyl]piperazine-1-carbonyl]-3-chlorophenyl]-1-[2-chloro-3-fluoro-4-(fluoromethoxy)phenyl]imidazole-4-carboxamide; 1:1 2,2,2-trifluoroacetic acid (19.8 mg, 0.03 mmol, 76.4% yield). MS [M+H]⁺: 633.7.

### Compound 106

### 5-Amino-N-[4-[4-[(1R,5S,6r)-3-azabicyclo[3.1.0]hexane-6-carbonyl]piperazine-1-carbonyl]-3-chlorophenyl]-2-chloro-1-[4-(fluoromethoxy)phenyl]imidazole-4-carboxamide; 1:1 formic acid

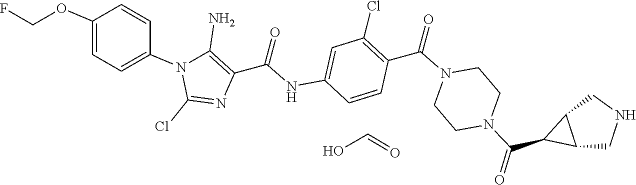

### Step 1: ethyl 5-amino-1-(4-methoxyphenyl)imidazole-4-carboxylate

A solution of ethyl 2-amino-2-cyanoacetate; 4-methylbenzene-1-sulfonic acid (9.75 g, 32.5 mmol, 2.0 eq), triethylamine (6.57 g, 65.0 mmol, 4.0 eq) and triethyl orthoformate (12.03 g, 81.2 mmol, 5.0 eq) in MeCN (40 mL) was heated at reflux for 2 h. After cooling to room temperature, p-anisidine (2.0 g, 16.24 mmol, 1.0 eq) was added. The mixture was stirred for 18 h at room temperature. After solvent removal under reduced pressure, the residue was diluted with EtOAc (3×40 mL), washed with H₂O (40 mL), dried over Na₂SO₄ and concentrated under reduced pressure. The residue was purified by flash column chromatography (30:70 petroleum ether:EtOAc) to give ethyl 5-amino-1-(4-methoxyphenyl)imidazole-4-carboxylate (700.0 mg, 2.68 mmol, 16.5% yield). MS [M+H]⁺: 261.9.

### Step 2: ethyl 5-amino-2-chloro-1-(4-methoxyphenyl)imidazole-4-carboxylate

To a solution of ethyl 5-amino-1-(4-methoxyphenyl)imidazole-4-carboxylate (100.0 mg, 0.38 mmol, 1.0 eq) in DMF (2 mL) was added N-chlorosuccinimide (51.11 mg, 0.38 mmol, 1.0 eq) under N₂ atmosphere. The mixture was stirred at 50° C. for 18 h, diluted with EtOAc (3×10 mL), washed with H₂O (20 mL), dried over Na₂SO₄ and concentrated under reduced pressure. The residue was purified by flash column chromatography (65:35 petroleum ether:EtOAc) to give ethyl 5-amino-2-chloro-1-(4-methoxyphenyl)imidazole-4-carboxylate (60.0 mg, 0.2 mmol, 52.5% yield). MS [M+H]⁺: 295.8.

### Step 3: 5-amino-2-chloro-1-(4-methoxyphenyl)imidazole-4-carboxylic acid

To a solution of ethyl 5-amino-2-chloro-1-(4-methoxyphenyl)imidazole-4-carboxylate (518.0 mg, 1.75 mmol, 1.0 eq) in ethanol (10 mL) were added sodium hydroxide (140.13 mg, 3.5 mmol, 2.0 eq) and water (5 mL) under N₂. The resulting mixture was stirred at 70° C. for 8 h. The reaction solution was cooled to room temperature and the pH of the reaction solution adjusted to 7-8. The mixture was then filtered to give 5-amino-2-chloro-1-(4-methoxyphenyl)imidazole-4-carboxylic acid (350.0 mg, 1.31 mmol, 74.6% yield). MS [M+H]⁺: 267.9.

### Step 4: 5-amino-2-chloro-1-(4-methoxyphenyl)imidazole-4-carbonyl chloride

To a solution of 5-amino-2-chloro-1-(4-methoxyphenyl)imidazole-4-carboxylic acid (50.0 mg, 0.19 mmol, 1.0 eq) in thionyl chloride (2.0 mL, 28 mmol, 150 eq) under N₂ atmosphere. The mixture was stirred at 0° C. for 1 h. The reaction mixture was concentrated under reduced pressure to give 5-amino-2-chloro-1-(4-methoxyphenyl)imidazole-4-carbonyl chloride (50.0 mg, 0.17 mmol, 93.6% yield). MS [(M−Cl+OCH₃)+H]⁺: 282.1.

### Step 5: tert-butyl (1R,5S)-6-[4-[4-[[5-amino-2-chloro-1-(4-methoxyphenyl)imidazole-4-carbonyl]amino]-2-chlorobenzoyl]piperazine-1-carbonyl]-3-azabicyclo[3.1.0]hexane-3-carboxylate

To a solution of tert-butyl (1R,5S)-6-[4-(4-amino-2-chlorobenzoyl)piperazine-1-carbonyl]-3-azabicyclo[3.1.0]hexane-3-carboxylate (470.74 mg, 1.05 mmol, 1.0 eq) in methylene chloride (3 mL) were added pyridine (248.83 mg, 3.15 mmol, 3.0 eq) and 5-amino-2-chloro-1-(4-methoxyphenyl)imidazole-4-carbonyl chloride (300.0 mg, 1.05 mmol, 1.0 eq) under N₂ atmosphere. The mixture was stirred at 0° C. for 1 h. The solvent was removed under reduced pressure and the residue was purified by flash column chromatography (90:10 methylene chloride:MeOH) to give tert-butyl (1R,5S)-6-[4-[4-[[5-amino-2-chloro-1-(4-methoxyphenyl)imidazole-4-carbonyl]amino]-2-chlorobenzoyl]piperazine-1-carbonyl]-3-azabicyclo[3.1.0]hexane-3-carboxylate (400.0 mg, 0.57 mmol, 52.4% yield). MS [M+H]⁺: 598.1.

### Step 6: 5-amino-N-[4-[4-[(1R,5S)-3-azabicyclo[3.1.0]hexane-6-carbonyl]piperazine-1-carbonyl]-3-chlorophenyl]-2-chloro-1-(4-hydroxyphenyl)imidazole-4-carboxamide

To a solution of tert-butyl (1R,5S)-6-[4-[4-[[5-amino-2-chloro-1-(4-methoxyphenyl)imidazole-4-carbonyl]amino]-2-chlorobenzoyl]piperazine-1-carbonyl]-3-azabicyclo[3.1.0]hexane-3-carboxylate (350.0 mg, 0.5 mmol, 1.0 eq) in methylene chloride (5 mL) was added boron tribromide (627.56 mg, 2.51 mmol, 5.0 eq) dropwise at 0° C. under N₂ atmosphere. The reaction mixture was allowed to warm to 25° C. and stirred for 5 h. Filtration of the reaction mixture afforded 5-amino-N-[4-[4-[(1R,5S)-3-azabicyclo[3.1.0]hexane-6-carbonyl]piperazine-1-carbonyl]-3-chlorophenyl]-2-chloro-1-(4-hydroxyphenyl)imidazole-4-carboxamide (230.0 mg, 0.39 mmol, 78.6% yield). MS [M+H]⁺: 584.2.

### Step 7: tert-butyl (1R,5S)-6-[4-[4-[[5-amino-2-chloro-1-(4-hydroxyphenyl)imidazole-4-carbonyl]amino]-2-chlorobenzoyl]piperazine-1-carbonyl]-3-azabicyclo[3.1.0]hexane-3-carboxylate

To a solution of 5-amino-N-[4-[4-[(1R,5S)-3-azabicyclo[3.1.0]hexane-6-carbonyl]piperazine-1-carbonyl]-3-chlorophenyl]-2-chloro-1-(4-hydroxyphenyl)imidazole-4-carboxamide (220.0 mg, 0.38 mmol, 1.0 eq) in THF (2 mL) was added saturated sodium bicarbonate aqueous solution (1.0 mL) at 25° C. under N₂ atmosphere. After stirring for 5 h, the reaction mixture was diluted with EtOAc (20 mL), washed with H₂O (3×20 mL), dried over Na₂SO₄ and concentrated under reduced pressure. The residue was purified by flash column chromatography (95:5 methylene chloride:MeOH) to give tert-butyl (1R,5S)-6-[4-[4-[[5-amino-2-chloro-1-(4-hydroxyphenyl)imidazole-4-carbonyl]amino]-2-chlorobenzoyl]piperazine-1-carbonyl]-3-azabicyclo[3.1.0]hexane-3-carboxylate (150.0 mg, 0.22 mmol, 53.6% yield). MS [M+H]⁺: 628.1.

### Step 8: tert-butyl (1R,5S)-6-[4-[4-[[5-amino-2-chloro-1-[4-(fluoromethoxy)phenyl]imidazole-4-carbonyl]amino]-2-chlorobenzoyl]piperazine-1-carbonyl]-3-azabicyclo[3.1.0]hexane-3-carboxylate

To a solution of tert-butyl (1R,5S)-6-[4-[4-[[5-amino-2-chloro-1-(4-hydroxyphenyl)imidazole-4-carbonyl]amino]-2-chlorobenzoyl]piperazine-1-carbonyl]-3-azabicyclo[3.1.0]hexane-3-carboxylate (70.0 mg, 0.1 mmol, 1.0 eq) in MeCN (2 mL) were added potassium carbonate (42.4 mg, 0.31 mmol, 3.0 eq) and fluoro(iodo)methane (32.71 mg, 0.2 mmol, 2.0 eq) under N₂ atmosphere. The vessel was sealed, and the mixture was stirred at 50° C. for 18 h. After concentration under reduced pressure, the residue was purified by flash column chromatography on silica gel (85:15 methylene chloride:MeOH) to yield tert-butyl (1R,5S)-6-[4-[4-[[5-amino-2-chloro-1-[4-(fluoromethoxy)phenyl]imidazole-4-carbonyl]amino]-2-chlorobenzoyl]piperazine-1-carbonyl]-3-azabicyclo[3.1.0]hexane-3-carboxylate (50.0 mg, 0.07 mmol, 60.7% yield). MS [(M−C₄H₈)+H]⁺: 660.1.

### Step 9: 5-amino-N-[4-[4-[(1R,5S)-3-azabicyclo[3.1.0]hexane-6-carbonyl]piperazine-1-carbonyl]-3-chloro-phenyl]-2-chloro-1-[4-(fluoromethoxy)phenyl]imidazole-4-carboxamide; 1:1 formic acid

To a solution of tert-butyl (1R,5S)-6-[4-[4-[[5-amino-2-chloro-1-[4-(fluoromethoxy)phenyl]imidazole-4-carbonyl]amino]-2-chlorobenzoyl]piperazine-1-carbonyl]-3-azabicyclo[3.1.0]hexane-3-carboxylate (20.0 mg, 0.03 mmol, 1.0 eq) in methylene chloride (2 mL) was added trifluoroacetic acid (0.25 mL, 3.4 mmol, 120 eq) under N₂ atmosphere. The resulting mixture was stirred at 25° C. After 2 h. The solvent was removed under reduced pressure and the residue was purified by prep-HPLC [Gemini-C18, 150×21.2 mm, 5 μm, MeCN-H₂O (0.1% FA), 15%˜40%] to give 5-amino-N-[4-[4-[(1R,5S)-3-azabicyclo[3.1.0]hexane-6-carbonyl]piperazine-1-carbonyl]-3-chloro-phenyl]-2-chloro-1-[4-(fluoromethoxy)phenyl]imidazole-4-carboxamide;formic acid (8.6 mg, 0.01 mmol, 25% yield). MS [M+H]⁺: 615.7

### Compound 107

### 5-amino-2-chloro-N-[3-chloro-4-[4-[(1R,5S,6r)-3-azabicyclo[3.1.0]hexane-6-carbonyl]piperazine-1-carbonyl]phenyl]-1-[2,3-difluoro-4-(4-hydroxybut-2-ynoxy)phenyl]imidazole-4-carboxamide; 1:1 2,2,2-trifluoroacetic acid

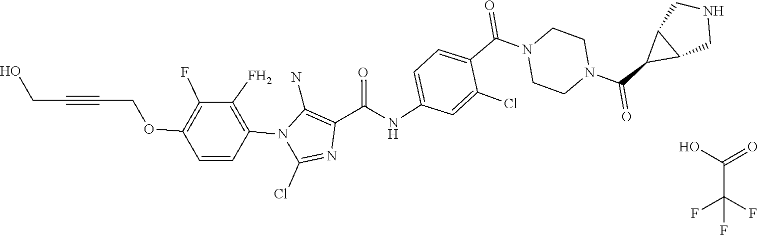

### Step 1: ethyl 5-amino-2-chloro-1-(2,3-difluoro-4-methoxyphenyl)imidazole-4-carboxylate

To a solution of ethyl 5-amino-1-(2,3-difluoro-4-methoxyphenyl)imidazole-4-carboxylate (500.0 mg, 1.68 mmol, 1.0 eq) in DMF (5 mL) was added N-chlorosuccinimide (269.52 mg, 2.02 mmol, 1.2 eq) under N₂ atmosphere. The mixture was stirred at 50° C. for 6 h and then diluted with EtOAc (3×10 mL), washed with H₂O (20 mL), dried over Na₂SO₄ and concentrated under reduced pressure. The residue was purified by flash column chromatography (65:35 petroleum ether:EtOAc) to give ethyl 5-amino-2-chloro-1-(2,3-difluoro-4-methoxyphenyl)imidazole-4-carboxylate (300.0 mg, 0.9 mmol, 52.2% yield). MS [M+H]⁺: 332.0.

### Step 2: ethyl 5-[bis(tert-butoxycarbonyl)amino]-2-chloro-1-(2,3-difluoro-4-methoxyphenyl)imidazole-4-carboxylate

To a solution of ethyl 5-amino-2-chloro-1-(2,3-difluoro-4-methoxyphenyl)imidazole-4-carboxylate (380.0 mg, 1.15 mmol, 1.0 eq) in methylene chloride (2 mL) were added triethylamine (695.55 mg, 6.87 mmol, 6.0 eq), di-tert-butyl dicarbonate (750.09 mg, 3.44 mmol, 3.0 eq) and 4-dimethylaminopyridine (279.92 mg, 2.29 mmol, 2.0 eq) at 25° C. under N₂ atmosphere. After stirring for 3 h, the reaction mixture was diluted with H₂O (3×20 mL), dried over Na₂SO₄ and concentrated under reduced pressure. The residue was purified by flash column chromatography (1:1 petroleum ether:EtOAc) to afford ethyl 5-[bis(tert-butoxycarbonyl)amino]-2-chloro-1-(2,3-difluoro-4-methoxyphenyl)imidazole-4-carboxylate (400.0 mg, 0.75 mmol, 61.0% yield). MS [M+H]⁺: 532.1.

### Step 3: 5-(tert-butoxycarbonylamino)-2-chloro-1-(2,3-difluoro-4-methoxyphenyl)imidazole-4-carboxylic acid

To a solution of ethyl 5-[bis(tert-butoxycarbonyl)amino]-2-chloro-1-(2,3-difluoro-4-methoxyphenyl)imidazole-4-carboxylate (400.0 mg, 0.75 mmol, 1.0 eq) in methanol (10 mL) were added LiOH·H₂O (157.77 mg, 3.76 mmol, 5.0 eq) and water (2.5 mL) under N₂. The resulting mixture was stirred at 25° C. for 72 h. After cooling to room temperature, the pH was adjusted to 7-8. Filtration of the mixture gave 5-(tert-butoxycarbonylamino)-2-chloro-1-(2,3-difluoro-4-methoxyphenyl)imidazole-4-carboxylic acid (260.0 mg, 0.64 mmol, 80.5% yield). MS [M+H]⁺: 426.0.

### Step 4: tert-butyl N-[2-chloro-5-chlorocarbonyl-3-(2,3-difluoro-4-methoxyphenyl) imidazol-4-yl]carbamate

To a solution of 5-(tert-butoxycarbonylamino)-2-chloro-1-(2,3-difluoro-4-methoxyphenyl)imidazole-4-carboxylic acid (200.0 mg, 0.5 mmol, 1.0 eq) in thionyl chloride (13.33 mL, 183.58 mmol, 370.61 eq) under N₂ atmosphere. The mixture was stirred at 0° C. for 1.5 h. The solvent was removed under reduced pressure to give tert-butyl N-[2-chloro-5-chlorocarbonyl-3-(2,3-difluoro-4-methoxyphenyl) imidazol-4-yl]carbamate (200.0 mg, 0.47 mmol, 75.6% yield). MS [(M−Cl+OCH₃)+H]⁺: 362.0.

### Step 5: tert-butyl (1R,5S)-6-[4-[4-[[5-amino-2-chloro-1-(2,3-difluoro-4-methoxyphenyl)imidazole-4-carbonyl]amino]-2-chlorobenzoyl]piperazine-1-carbonyl]-3-azabicyclo[3.1.0]hexane-3-carboxylate

A solution of tert-butyl (1R,5S)-6-[4-(4-amino-2-chlorobenzoyl)piperazine-1-carbonyl]-3-azabicyclo[3.1.0]hexane-3-carboxylate (221.17 mg, 0.49 mmol, 1.0 eq) and pyridine (0.57 mL, 7.0 mmol, 14 eq) in methylene chloride (2 mL) was stirred at 0° C. This was followed by the addition of a solution of tert-butyl N-[2-chloro-5-chlorocarbonyl-3-(2,3-difluoro-4-methoxyphenyl) imidazol-4-yl]carbamate (260.0 mg, 0.49 mmol, 1.0 eq) in methylene chloride (2 mL). Next, the temperature was increased to 25° C. for 30 min. The solvent was then removed under reduced pressure, and the residue was purified by flash column chromatography (90:10 methylene chloride:MeOH) to give tert-butyl (1R,5S)-6-[4-[4-[[5-amino-2-chloro-1-(2,3-difluoro-4-methoxyphenyl)imidazole-4-carbonyl]amino]-2-chlorobenzoyl]piperazine-1-carbonyl]-3-azabicyclo[3.1.0]hexane-3-carboxylate (200.0 mg, 0.27 mmol, 52.5% yield). MS [M+H]⁺: 678.1.

### Step 6: 5-amino-N-[4-[4-[(1R,5S)-3-azabicyclo[3.1.0]hexane-6-carbonyl]piperazine-1-carbonyl]-3-chloro-phenyl]-2-chloro-1-(2,3-difluoro-4-hydroxy-phenyl)imidazole-4-carboxamide

A solution of tert-butyl (1R,5S)-6-[4-[4-[[5-amino-2-chloro-1-(2,3-difluoro-4-methoxyphenyl)imidazole-4-carbonyl]amino]-2-chlorobenzoyl]piperazine-1-carbonyl]-3-azabicyclo[3.1.0]hexane-3-carboxylate (90.0 mg, 0.12 mmol, 1.0 eq) in boron tribromide (5.0 mL, 52 mmol, 420 eq) was stirred at 60° C. for 32 h. The solvent was removed under reduced pressure to give 5-amino-N-[4-[4-[(1R,5S)-3-azabicyclo[3.1.0]hexane-6-carbonyl]piperazine-1-carbonyl]-3-chlorophenyl]-2-chloro-1-(2,3-difluoro-4-hydroxy-phenyl)imidazole-4-carboxamide (90.0 mg, 0.15 mmol, 95.9% yield). The crude product was used directly without further purification. MS [M+H]⁺: 620.2.

### Step 7: tert-butyl (1R,5S)-6-[4-[4-[[5-amino-2-chloro-1-(2,3-difluoro-4-hydroxyphenyl)imidazole-4-carbonyl]amino]-2-chlorobenzoyl]piperazine-1-carbonyl]-3-azabicyclo[3.1.0]hexane-3-carboxylate

To a solution of 5-amino-N-[4-[4-[(1R,5S)-3-azabicyclo[3.1.0]hexane-6-carbonyl]piperazine-1-carbonyl]-3-chlorophenyl]-2-chloro-1-(2,3-difluoro-4-hydroxyphenyl)imidazole-4-carboxamide (90.0 mg, 0.15 mmol, 1.0 eq) in THF (2 mL) was added saturated sodium bicarbonate aqueous solution (1 mL) at 25° C. under N₂ atmosphere. After stirring for 2 h, the reaction mixture was diluted with EtOAc (20 mL), washed with H₂O (3×20 mL), dried over Na₂SO₄ and concentrated under reduced pressure. The residue was purified by flash column chromatography (90:10 methylene chloride:MeOH) to give tert-butyl (1R,5S)-6-[4-[4-[[5-amino-2-chloro-1-(2,3-difluoro-4-hydroxyphenyl)imidazole-4-carbonyl]amino]-2-chlorobenzoyl]piperazine-1-carbonyl]-3-azabicyclo[3.1.0]hexane-3-carboxylate (90.0 mg, 0.12 mmol, 70.6% yield). MS [M+H]⁺: 664.0.

### Step 8: tert-butyl (1R,5S)-6-[4-[4-[[5-amino-1-[4-[4-[tert-butyl(diphenyl)silyl]oxybut-2-ynoxy]-2,3-difluoro-phenyl]-2-chloroimidazole-4-carbonyl]amino]-2-chlorobenzoyl]piperazine-1-carbonyl]-3-azabicyclo[3.1.0]hexane-3-carboxylate

A solution of tert-butyl (1R,5S)-6-[4-[4-[[5-amino-2-chloro-1-(2,3-difluoro-4-hydroxyphenyl)imidazole-4-carbonyl]amino]-2-chlorobenzoyl]piperazine-1-carbonyl]-3-azabicyclo[3.1.0]hexane-3-carboxylate (80.0 mg, 0.11 mmol, 1.0 eq), potassium carbonate (46.03 mg, 0.33 mmol, 3.0 eq) and 4-[tert-butyl(diphenyl)silyl]oxybut-2-ynyl methanesulfonate (49.17 mg, 0.12 mmol, 1.1 eq) in DMF (2 mL) was stirred at 25° C. for 16 h. The reaction mixture was then diluted with EtOAc (10 mL), washed with H₂O (3×10 mL), dried over Na₂SO₄ and concentrated under reduced pressure. The residue was purified by flash column chromatography (95:5 methylene chloride:MeOH elution) to yield tert-butyl (1R,5S)-6-[4-[4-[[5-amino-1-[4-[4-[tert-butyl(diphenyl)silyl]oxybut-2-ynoxy]-2,3-difluorophenyl]-2-chloroimidazole-4-carbonyl]amino]-2-chlorobenzoyl]piperazine-1-carbonyl]-3-azabicyclo[3.1.0]hexane-3-carboxylate (70.0 mg, 0.07 mmol, 56.5% yield). MS [(M−Boc)+H]⁺: 926.2.

### Step 9: 5-amino-1-[4-[4-[tert-butyl(diphenyl)silyl]oxybut-2-ynoxy]-2,3-difluorophenyl]-2-chloro-N-[3-chloro-4-[4-[(1R,5S)-3-azabicyclo[3.1.0]hexane-6-carbonyl]piperazine-1-carbonyl]phenyl]imidazole-4-carboxamide

To a solution of tert-butyl (1R,5S)-6-[4-[4-[[5-amino-1-[4-[4-[tert-butyl(diphenyl)silyl]oxybut-2-ynoxy]-2,3-difluorophenyl]-2-chloroimidazole-4-carbonyl]amino]-2-chlorobenzoyl]piperazine-1-carbonyl]-3-azabicyclo[3.1.0]hexane-3-carboxylate (50.0 mg, 0.05 mmol, 1.0 eq) in methylene chloride (2 mL) was added trifluoroacetic acid (1.0 mL, 13.46 mmol, 276.53 eq). The resulting mixture was stirred at 25° C. for 12 h. Concentration under reduced pressure afforded 5-amino-1-[4-[4-[tert-butyl(diphenyl)silyl]oxybut-2-ynoxy]-2,3-difluorophenyl]-2-chloro-N-[3-chloro-4-[4-[(1R,5S)-3-azabicyclo[3.1.0]hexane-6-carbonyl]piperazine-1-carbonyl]phenyl]imidazole-4-carboxamide (50.0 mg, 0.05 mmol, 99.7% yield). The crude product was used directly without further purification. MS [M+H]⁺: 926.1.

### Step 10: 5-amino-2-chloro-N-[3-chloro-4-[4-[(1R,5S)-3-azabicyclo[3.1.0]hexane-6-carbonyl]piperazine-1-carbonyl]phenyl]-1-[2,3-difluoro-4-(4-hydroxybut-2-ynoxy)phenyl]imidazole-4-carboxamide; 1:1 2,2,2-trifluoroacetic acid

To a solution of 5-amino-1-[4-[4-[tert-butyl(diphenyl)silyl]oxybut-2-ynoxy]-2,3-difluorophenyl]-2-chloro-N-[3-chloro-4-[4-[(1R,5S)-3-azabicyclo[3.1.0]hexane-6-carbonyl]piperazine-1-carbonyl]phenyl]imidazole-4-carboxamide (40.0 mg, 0.04 mmol, 1.0 eq) in DMF (3 mL) was added cesium fluoride (32.78 mg, 0.22 mmol, 5.0 eq) under N₂ atmosphere. The resulting mixture was stirred at 25° C. for 5 h. After solvent removal under reduced pressure, the residue was purified by prep-HPLC [Gemini-C18, 150×21.2 mm, 5 μm, MeCN-H₂O (0.1% TFA), 15%˜40%] to give 5-amino-2-chloro-N-[3-chloro-4-[4-[(1R,5S)-3-azabicyclo[3.1.0]hexane-6-carbonyl]piperazine-1-carbonyl]phenyl]-1-[2,3-difluoro-4-(4-hydroxybut-2-ynoxy)phenyl]imidazole-4-carboxamide; 1:1 2,2,2-trifluoroacetic acid (24.2 mg, 0.03 mmol, 69.9% yield). MS [M+H]⁺: 688.0.

### Compound 108

### 5-amino-1-[2-chloro-3-fluoro-4-(fluoromethoxy)phenyl]-N-[3-fluoro-4-[4-(piperidine-4-carbonyl)piperazine-1-carbonyl]phenyl]imidazole-4-carboxamide; 1:1 2,2,2-trifluoroacetic acid

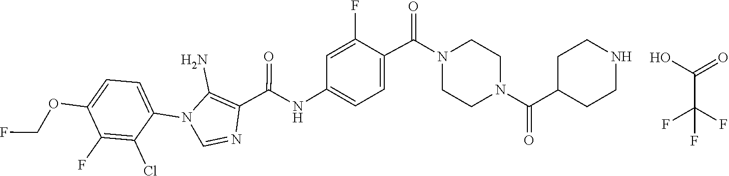 [SMILES: Nc1c(C(=O)Nc2ccc(C(=O)N3CCN(C(=O)C4CCNCC4)CC3)c(F)c2)ncn1-c1ccc(OCF)c(F)c1Cl ; O=C(O)C(F)(F)F]

### Step 1: 5-amino-1-[2-chloro-3-fluoro-4-(fluoromethoxy)phenyl]imidazole-4-carbonyl chloride

A solution of 5-amino-1-[2-chloro-3-fluoro-4-(fluoromethoxy)phenyl]imidazole-4-carboxylic acid (100.0 mg, 0.33 mmol, 1.0 eq) in thionyl chloride (4.0 mL, 55 mmol, 170 eq) was stirred at 0° C. for 1 h under N₂ atmosphere. The solvent was removed under reduced pressure to give 5-amino-1-[2-chloro-3-fluoro-4-(fluoromethoxy)phenyl]imidazole-4-carbonyl chloride (100.0 mg, 0.31 mmol, 89.56% yield). MS [(M−Cl+OCH₃)+H]⁺: 318.0.

### Step 2: tert-butyl 4-[4-[[5-amino-1-[2-chloro-3-fluoro-4-(fluoromethoxy)phenyl]imidazole-4-carbonyl]amino]-2-fluorobenzoyl]piperazine-1-carboxylate

A solution of tert-butyl 4-(4-amino-2-fluorobenzoyl)piperazine-1-carboxylate (100.39 mg, 0.31 mmol, 1.0 eq) and pyridine (73.67 mg, 0.93 mmol, 3.0 eq) in methylene chloride (2 mL) was stirred at 0° C. To the mixture was added 5-amino-1-[2-chloro-3-fluoro-4-(fluoromethoxy)phenyl]imidazole-4-carbonyl chloride (100.0 mg, 0.31 mmol, 1.0 eq) under N₂ atmosphere. The mixture was stirred at 25° C. for 16 h. Concentration under reduced pressure followed by purification with flash column chromatography on silica gel (90:10 methylene chloride:MeOH) afforded tert-butyl 4-[4-[[5-amino-1-[2-chloro-3-fluoro-4-(fluoromethoxy)phenyl]imidazole-4-carbonyl]amino]-2-fluorobenzoyl]piperazine-1-carboxylate (150.0 mg, 0.25 mmol, 61.01% yield). MS [M+H]⁺: 609.2.

### Step 3: 5-amino-1-[2-chloro-3-fluoro-4-(fluoromethoxy)phenyl]-N-[3-fluoro-4-(piperazine-1-carbonyl)phenyl]imidazole-4-carboxamide

To a solution of tert-butyl 4-[4-[[5-amino-1-[2-chloro-3-fluoro-4-(fluoromethoxy)phenyl]imidazole-4-carbonyl]amino]-2-fluorobenzoyl]piperazine-1-carboxylate (100.0 mg, 0.16 mmol, 1.0 eq) in methylene chloride (2 mL) was added hydrogen chloride (4 M in dioxane)(0.4 mL, 1.6 mmol, 9.7 eq). The resulting mixture was stirred at 25° C. for 1 h. The solvent was then removed under reduced pressure to give 5-amino-1-[2-chloro-3-fluoro-4-(fluoromethoxy)phenyl]-N-[3-fluoro-4-(piperazine-1-carbonyl)phenyl]imidazole-4-carboxamide (70.0 mg, 0.14 mmol, 81.3% yield). MS [M+H]⁺: 509.1.

### Step 4: tert-butyl 4-[4-[4-[[5-amino-1-[2-chloro-3-fluoro-4-(fluoromethoxy)phenyl]imidazole-4-carbonyl]amino]-2-fluorobenzoyl]piperazine-1-carbonyl]piperidine-1-carboxylate

To a solution of 5-amino-1-[2-chloro-3-fluoro-4-(fluoromethoxy)phenyl]-N-[3-fluoro-4-(piperazine-1-carbonyl)phenyl]imidazole-4-carboxamide (60.0 mg, 0.12 mmol, 1.0 eq) in methylene chloride (2 mL) were added N-BOC-isonipecotic acid (32.44 mg, 0.14 mmol, 1.2 eq), 2-(7-azabenzotriazol-1-yl)-N,N,N′,N′-tetramethyluronium hexafluorophosphate (134.5 mg, 0.35 mmol, 3.0 eq) and N,N-diisopropylethylamine (45.71 mg, 0.35 mmol, 3.0 eq). The resulting mixture was stirred at 25° C. for 3 h. The solvent was then removed under reduced pressure, and the residue was dissolved in EtOAc (30 mL) and washed with H₂O (3×15 mL). The combined organic phases were dried over Na₂SO₄ and concentrated. The crude material was purified by flash column chromatography on silica gel (90:10 methylene chloride:MeOH) to provide tert-butyl 4-[4-[4-[[5-amino-1-[2-chloro-3-fluoro-4-(fluoromethoxy)phenyl]imidazole-4-carbonyl]amino]-2-fluorobenzoyl]piperazine-1-carbonyl]piperidine-1-carboxylate (60.0 mg, 0.08 mmol, 65.7% yield). MS [M+H]⁺: 720.1.

### Step 5: 5-amino-1-[2-chloro-3-fluoro-4-(fluoromethoxy)phenyl]-N-[3-fluoro-4-[4-(piperidine-4-carbonyl)piperazine-1-carbonyl]phenyl]imidazole-4-carboxamide; 1:1 2,2,2-trifluoroacetic acid

To a solution of tert-butyl 4-[4-[4-[[5-amino-1-[2-chloro-3-fluoro-4-(fluoromethoxy)phenyl]imidazole-4-carbonyl]amino]-2-fluorobenzoyl]piperazine-1-carbonyl]piperidine-1-carboxylate (50.0 mg, 0.07 mmol, 1.0 eq) in methylene chloride (2 mL) was added trifluoroacetic acid (0.44 mL, 6.0 mmol, 86 eq) under N₂ atmosphere. The resulting mixture was stirred at 25° C. After 3 h the solvent was removed under reduced pressure, and the residue was purified by prep-HPLC [Gemini-C18, 150×21.2 mm, 5 μm, CAN-H₂O (0.1% FA), 15%˜40%] to give 5-amino-1-[2-chloro-3-fluoro-4-(fluoromethoxy)phenyl]-N-[3-fluoro-4-[4-(piperidine-4-carbonyl)piperazine-1-carbonyl]phenyl]imidazole-4-carboxamide; 1:1 2,2,2-trifluoroacetic acid (33.97 mg, 0.05 mmol, 78.8% yield). MS [M+H]⁺: 620.1.

### Compound 109

### 5-amino-N-[4-[4-[(1S,5R)-3-azabicyclo[3.1.0]hexane-6-carbonyl]piperazine-1-carbonyl]-3-fluorophenyl]-1-[2-chloro-4-(fluoromethoxy)-3-methoxyphenyl]imidazole-4-carboxamide; 1:1 2,2,2-trifluoroacetic acid

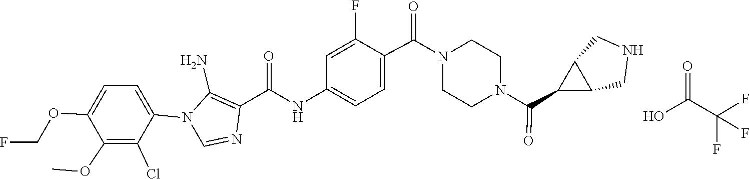 [SMILES: COc1c(OCF)ccc(-n2cnc(C(=O)Nc3ccc(C(=O)N4CCN(C(=O)[C@H]5[C@@H]6CNC[C@@H]65)CC4)c(F)c3)c2N)c1Cl ; O=C(O)C(F)(F)F]

### Step 1: 2-chloro-6-fluoro-3-nitrophenol

To a solution of 2-fluoro-5-nitrophenol (2000.0 mg, 12.73 mmol, 1.0 eq) in toluene (20 mL) was added diisobutylamine (164.53 mg, 1.27 mmol, 0.1 eq) and sulfuryl chloride (1804.18 mg, 13.37 mmol, 1.05 eq). The reaction was stirred at 70° C. for 72 h. The reaction was then quenched with H₂O (50 mL) and extracted with EtOAc (2×50 mL). The organic layers were combined, washed with brine, dried and concentrated under reduced pressure. The residue was purified by flash chromatography with petroleum ether:EtOAc elution (100:1 to 1:1) to afford 2-chloro-6-fluoro-3-nitrophenol (700.0 mg, 3.65 mmol, 28.7% yield). MS [M−H]⁺: 190.0.

### Step 2: 3-chloro-1-fluoro-2-methoxy-4-nitrobenzene

To a solution of 2-chloro-6-fluoro-3-nitrophenol (700.0 mg, 3.65 mmol, 1.0 eq) in MeCN (10 mL) were added potassium carbonate (1515.26 mg, 10.96 mmol, 3.0 eq) and methyl iodide (1037.46 mg, 7.31 mmol, 2.0 eq) under N₂ atmosphere. The vessel was sealed and the mixture was stirred at 40° C. for 2 h. The reaction was then concentrated under reduced pressure. The residue was purified by flash column chromatography on silica gel (95:5 petroleum ether:EtOAc) to give 3-chloro-1-fluoro-2-methoxy-4-nitrobenzene (500.0 mg, 2.43 mmol, 66.6% yield).

### Step 3: 3-chloro-2-methoxy-4-nitrophenol

A solution of 3-chloro-1-fluoro-2-methoxy-4-nitrobenzene (500.0 mg, 2.43 mmol, 1.0 eq), potassium hydroxide (341.16 mg, 6.08 mmol, 2.5 eq) in DMSO (5 mL) was heated at 80° C. for 4 h. The resulting solution was diluted with 10 ml of water, then extracted with 3×10 mL of EtOAc. The organic layers were combined, washed with brine, dried and concentrated under reduced pressure. The residue was applied on a silica gel column and eluted with petroleum ether/EtOAc (10:1 to 100% EtOAc) to give 3-chloro-2-methoxy-4-nitrophenol (400.0 mg, 1.96 mmol, 80.8% yield). MS [M−H]⁺: 202.0.

### Step 4: 3-chloro-1-(fluoromethoxy)-2-methoxy-4-nitrobenzene

To a solution of 3-chloro-2-methoxy-4-nitrophenol (350.0 mg, 1.72 mmol, 1.0 eq) in MeCN (3 mL) were added N,N-diisopropylethylamine (666.58 mg, 5.16 mmol, 3.0 eq) and fluoro(iodo)methane (412.43 mg, 2.58 mmol, 1.5 eq) under N₂ atmosphere. The vessel was sealed and the mixture was stirred at 50° C. for 15 h. Concentration under reduced pressure followed by purification with flash column chromatography on silica gel (95:5 petroleum ether:EtOAc) to give 3-chloro-1-(fluoromethoxy)-2-methoxy-4-nitrobenzene (320.0 mg, 1.36 mmol, 63.2% yield).

### Step 5: 2-chloro-4-(fluoromethoxy)-3-methoxyaniline

A mixture of 3-chloro-1-(fluoromethoxy)-2-methoxy-4-nitrobenzene (320.0 mg, 1.36 mmol, 1.0 eq), ammonium chloride (363.26 mg, 6.79 mmol, 5.0 eq) and iron powder (380.31 mg, 6.79 mmol, 5.0 eq) in ethanol (3 mL) and water (3 mL) was heated at 60° C. for 4 h. The mixture was cooled to room temperature and filtered through a short plug of Celite (200 mg), rinsing the solid support with methanol (30 mL), the solvent was removed under reduced pressure to produce a oil, which was dissolved with ethyl acetate (30 mL), and washed with water, dried (Na₂SO₄) and evaporated to dryness to afford 2-chloro-4-(fluoromethoxy)-3-methoxyaniline (250.0 mg, 1.22 mmol, 89.5% yield). MS [M+H]⁺: 206.0.

### Step 6: ethyl 5-amino-1-[2-chloro-4-(fluoromethoxy)-3-methoxyphenyl]imidazole-4-carboxylate

To a solution of ethyl 2-amino-2-cyanoacetate, 4-methylbenzenesulfonic acid (1:1)(1007.87 mg, 3.36 mmol, 3.0 eq) in MeCN (8 mL) were added triethylamine (0.94 mL, 6.7 mmol, 6.0 eq) and triethyl orthoformate (1657.8 mg, 11.19 mmol, 10.0 eq). The reaction mixture was stirred at 80° C. for 2 h. The solvent was then removed under reduced pressure. The residue was diluted with MeCN (8 mL), then 2-chloro-4-(fluoromethoxy)-3-methoxyaniline (230.0 mg, 1.12 mmol, 1.0 eq) was added and the resulting mixture was stirred at 40° C. for 15 h. Concentration under reduced pressure and purification by flash column chromatography on silica gel (1:3 petroleum ether:EtOAc) afforded ethyl 5-amino-1-[2-chloro-4-(fluoromethoxy)-3-methoxyphenyl]imidazole-4-carboxylate (300.0 mg, 0.87 mmol, 78.0% yield). MS [M+H]⁺: 344.0.

### Step 7: 5-amino-1-[2-chloro-4-(fluoromethoxy)-3-methoxyphenyl]imidazole-4-carboxylic acid

A solution of ethyl 5-amino-1-[2-chloro-4-(fluoromethoxy)-3-methoxyphenyl]imidazole-4-carboxylate (100.0 mg, 0.29 mmol, 1.0 eq), potassium hydroxide (81.61 mg, 1.45 mmol, 5.0 eq) in the isopropyl alcohol (9 mL) and water (3 mL) was stirred at 70° C. for 6 h. The residue was diluted with 100 ml of water, then adjusted to pH 6˜7 with HCl (2 M) and extracted with 3×100 ml of EtOAc. The organic layers were combined and concentrated under reduced pressure to give 5-amino-1-[2-chloro-4-(fluoromethoxy)-3-methoxyphenyl]imidazole-4-carboxylic acid (75.0 mg, 0.24 mmol, 73.5% yield). MS [M+H]⁺: 316.0.

### Step 8: 5-amino-1-[2-chloro-4-(fluoromethoxy)-3-methoxyphenyl]imidazole-4-carbonyl chloride

A solution of 5-amino-1-[2-chloro-4-(fluoromethoxy)-3-methoxyphenyl]imidazole-4-carboxylic acid (50.0 mg, 0.16 mmol, 1.0 eq) in thionyl chloride (2.0 mL, 27 mmol, 170 eq) was stirred at 25° C. for 1 h. The resulting solution was concentrated under reduced pressure to give 5-amino-1-[2-chloro-4-(fluoromethoxy)-3-methoxyphenyl]imidazole-4-carbonyl chloride (50.0 mg, 0.15 mmol, 94.5% yield). MS [M−Cl+OCH₃+H]⁺: 330.0.

### Step 9: tert-butyl (1S,5R)-6-[4-[4-[[5-amino-1-[2-chloro-4-(fluoromethoxy)-3-methoxyphenyl]imidazole-4-carbonyl]amino]-2-fluorobenzoyl]piperazine-1-carbonyl]-3-azabicyclo[3.1.0]hexane-3-carboxylate

A solution of tert-butyl (1S,5R)-6-[4-(4-amino-2-fluorobenzoyl)piperazine-1-carbonyl]-3-azabicyclo[3.1.0]hexane-3-carboxylate (64.72 mg, 0.15 mmol, 1.0 eq) and pyridine (0.04 mL, 0.45 mmol, 3.0 eq) in methylene chloride (2 mL) was stirred at 25° C. Next, 5-amino-1-[2-chloro-4-(fluoromethoxy)-3-methoxyphenyl]imidazole-4-carbonyl chloride (50.0 mg, 0.15 mmol, 1.0 eq) in methylene chloride (2 mL) was added, and the temperature was increased to 25° C. for 1 h. The resulting solution was concentrated under reduced pressure. The residue was purified on a silica gel column with 10:1 methylene chloride:MeOH elution to afford tert-butyl (1S,5R)-6-[4-[4-[[5-amino-1-[2-chloro-4-(fluoromethoxy)-3-methoxyphenyl]imidazole-4-carbonyl]amino]-2-fluorobenzoyl]piperazine-1-carbonyl]-3-azabicyclo[3.1.0]hexane-3-carboxylate (70.0 mg, 0.1 mmol, 51.2% yield). MS [M+H]⁺: 730.3.

### Step 10: 5-amino-N-[4-[4-[(1S,5R)-3-azabicyclo[3.1.0]hexane-6-carbonyl]piperazine-1-carbonyl]-3-fluorophenyl]-1-[2-chloro-4-(fluoromethoxy)-3-methoxyphenyl]imidazole-4-carboxamide; 1;1 2,2,2-trifluoroacetic acid

A solution of tert-butyl (1S,5R)-6-[4-[4-[[5-amino-1-[2-chloro-4-(fluoromethoxy)-3-methoxyphenyl]imidazole-4-carbonyl]amino]-2-fluorobenzoyl]piperazine-1-carbonyl]-3-azabicyclo[3.1.0]hexane-3-carboxylate (60.0 mg, 0.08 mmol, 1.0 eq), trifluoroacetic acid (1.5 mL, 19.5 mmol, 237 eq) in methylene chloride (1 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40) to afford 5-amino-N-[4-[4-[(1S,5R)-3-azabicyclo[3.1.0]hexane-6-carbonyl]piperazine-1-carbonyl]-3-fluorophenyl]-1-[2-chloro-4-(fluoromethoxy)-3-methoxyphenyl]imidazole-4-carboxamide;1:1 2,2,2-trifluoroacetic acid (33.0 mg, 0.04 mmol, 54% yield). MS [M+H]⁺: 631.4.

### Compound 110

### N-(4-(4-((1R,5S,6r)-3-azabicyclo[3.1.0]hexane-6-carbonyl)piperazine-1-carbonyl)-3-fluorophenyl)-5-amino-1-(2-fluoro-4-(fluoromethoxy)-3-methoxyphenyl)-1H-imidazole-4-carboxamide, 1:1 2,2,2-trifluoroacetate

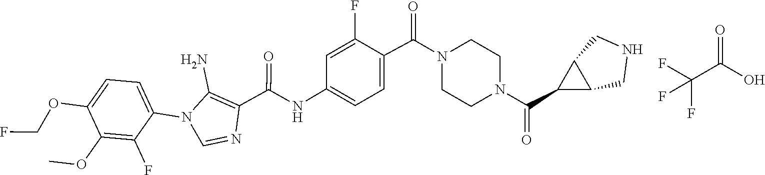

### Step 1: 4-bromo-2-fluoro-3-methoxyaniline

To a solution of 2-fluoro-3-methoxyaniline (2000.0 mg, 14.17 mmol, 1.0 eq) in DMF (20 mL) was added N-bromosuccinimide (2522.03 mg, 14.17 mmol, 1.0 eq). The reaction was stirred at 25° C. for 16 h and then concentrated to dryness. The residue was taken up in EtOAc (30 mL), and the organics were washed with 2×30 ml water and 30 mL brine. The organics were then separated and dried (MgSO₄) before concentrating to dryness. The crude was then purified by flash column chromatography eluting with 40% EtOAc in petroleum ether to yield 4-bromo-2-fluoro-3-methoxyaniline (2000.0 mg, 9.09 mmol, 64.1% yield). MS [M+H]⁺: 219.9.

### Step 2: tert-butyl (4-bromo-2-fluoro-3-methoxyphenyl) carbamate

To a solution of 4-bromo-2-fluoro-3-methoxyaniline (2000.0 mg, 9.09 mmol, 1.0 eq) in THF (3 mL) was added di-tert-butyl dicarbonate (2975.55 mg, 13.63 mmol, 1.5 eq) and sodium carbonate (2890.06 mg, 27.27 mmol, 3.0 eq). The reaction was stirred at 25° C. for 5 h and then concentrated to dryness. The residue was taken up in EtOAc (50 mL) and washed with 2×30 mL water followed by 30 mL brine. The organics were then separated and dried (MgSO₄) before concentrating to dryness. The crude was then purified by flash column chromatography eluting with 1% EtOAc in petroleum ether elution to afford tert-butyl N-(4-bromo-2-fluoro-3-methoxyphenyl) carbamate (1800.0 mg, 5.62 mmol, 61.9% yield). MS [M−Boc+H]⁺: 220.0.

### Step 3: tert-butyl (2-fluoro-3-methoxy-4-(4,4,5,5-tetramethyl-1,3,2-dioxaborolan-2-yl)phenyl) carbamate

A solution of tert-butyl N-(4-bromo-2-fluoro-3-methoxyphenyl) carbamate (1800.0 mg, 5.62 mmol, 1.0 eq) in 1,4-dioxane (20 mL) was added potassium acetate (1655.34 mg, 16.87 mmol, 3.0 eq) and [1,1′-bis(diphenylphosphino)ferrocene]palladium (II) chloride (815.99 mg, 1.12 mmol, 0.2 eq) and bis(pinacolato)diboron (2141.61 mg, 8.43 mmol, 1.5 eq) was stirred at 100° C. for 12 h under nitrogen. The reaction was then concentrated to dryness. The crude was then purified by flash column chromatography with 1% EtOAc in petroleum ether elution to obtain tert-butyl N-[2-fluoro-3-methoxy-4-(4,4,5,5-tetramethyl-1,3,2-dioxaborolan-2-yl)phenyl]carbamate (700.0 mg, 1.91 mmol, 25.8% yield). MS [M+Na]⁺: 390.1.

### Step 4: tert-butyl (2-fluoro-3-methoxy-4-(4,4,5,5-tetramethyl-1,3,2-dioxaborolan-2-yl)phenyl) carbamate

To a solution of tert-butyl N-[2-fluoro-3-methoxy-4-(4,4,5,5-tetramethyl-1,3,2-dioxaborolan-2-yl)phenyl]carbamate (630.0 mg, 1.72 mmol, 1.0 eq) in ethanol (8 mL) was added hydrogen peroxide (8.0 mL, 1.7 mmol, 1.0 eq). The reaction was stirred at 50° C. for 16 h. The residue was taken up in EtOAc (30 mL) and the organics washed with 2×20 mL water followed by 20 mL brine. The organics were then separated and dried (MgSO₄) before concentration under reduced pressure. The crude material was then purified by flash column chromatography eluting with with 5% EtOAc in petroleum ether to afford tert-butyl N-(2-fluoro-4-hydroxy-3-methoxyphenyl) carbamate (220.0 mg, 0.86 mmol, 49.8% yield). MS [M−t−Bu+H]⁺: 202.1.

### Step 5: tert-butyl (2-fluoro-4-(fluoromethoxy)-3-methoxyphenyl) carbamate

To a solution of tert-butyl N-(2-fluoro-4-hydroxy-3-methoxyphenyl) carbamate (200.0 mg, 0.78 mmol, 1.0 eq) in MeCN (3 mL) was added N, N-diisopropylethylamine (300.86 mg, 2.33 mmol, 3.0 eq) and iodomethane (331.04 mg, 2.33 mmol, 3.0 eq). The reaction was stirred at 50° C. for 12 h and then concentrated to dryness. The residue was taken up in EtOAc (10 mL) and the organics washed with 2×10 mL water and 10 mL brine. The organics were then separated and dried (MgSO₄) before concentrating to dryness. The crude material was then purified by flash column chromatography eluting with with 40% EtOAc in petroleum ether to afford tert-butyl N-[2-fluoro-4-(fluoromethoxy)-3-methoxyphenyl]carbamate (124.0 mg, 0.43 mmol, 46.67% yield). MS [M−tBu+H]⁺: 234.1.

### Step 6: 2-fluoro-4-(fluoromethoxy)-3-methoxyaniline

To a solution of tert-butyl N-[2-fluoro-4-(fluoromethoxy)-3-methoxyphenyl]carbamate (114.0 mg, 0.39 mmol, 1.0 eq) in methylene chloride (3 mL) was added trifluoroacetic acid (1.0 mL). The reaction was stirred at 25° C. for 3 h and then concentrated to dryness to afford crude product 2-fluoro-4-(fluoromethoxy)-3-methoxyaniline (65.0 mg, 0.34 mmol, 84.6% yield) MS [M+H]⁺: 190.1.

### Step 7: ethyl 5-amino-1-(2-fluoro-4-(fluoromethoxy)-3-methoxyphenyl)-1H-imidazole-4-carboxylate

To a solution of ethyl 2-amino-2-cyanoacetate; 4-methylbenzene-1-sulfonic acid (206.4 mg, 0.69 mmol, 2.0 eq) in triethyl orthoformate (2.49 mL, 14.91 mmol, 43.4 eq) was added triethylamine (139.09 mg, 1.37 mmol, 4.0 eq). The resulting mixture was stirred at 82° C. for 2 h, after which time the solvent was removed under reduced pressure. The residue was diluted with MeCN (20 mL), and then 2-fluoro-4-(fluoromethoxy)-3-methoxyaniline (65.0 mg, 0.34 mmol, 1.0 eq) was added. The resulting mixture was stirred at 25° C. for 15 h. The reaction was then concentrated to dryness. The residue was taken up in EtOAc (30 mL) and the organics washed with 2×30 mL water and 30 mL brine. The organics were then separated and dried (MgSO₄) before concentrating to dryness. The crude material was then purified by flash column chromatography eluting with with 3% MeOH in methylene chloride to afford ethyl 5-amino-1-[2-fluoro-4-(fluoromethoxy)-3-methoxyphenyl]imidazole-4-carboxylate (60.0 mg, 0.18 mmol, 53.4% yield). MS [M+H]⁺: 328.1.

### Step 8: 5-amino-1-[2-fluoro-4-(fluoromethoxy)-3-methoxyphenyl]imidazole-4-carboxylic acid

To a solution of ethyl 5-amino-1-[2-fluoro-4-(fluoromethoxy)-3-methoxyphenyl]imidazole-4-carboxylate (50.0 mg, 0.15 mmol, 1.0 eq) in 1-propanol (3 mL) and water (1.0 mL) added sodium hydroxide (30.55 mg, 0.76 mmol, 5.0 eq). The reaction was stirred at 70° C. for 4 h and then concentrated to dryness. The residue was taken up in water and then adjusted to pH 6˜7 with 1 N HCl. Filtration afforded the residue 5-amino-1-[2-fluoro-4-(fluoromethoxy)-3-methoxyphenyl]imidazole-4-carboxylic acid (30.0 mg, 0.1 mmol, 65.6% yield). MS [M+H]⁺: 300.1.

### Step 9: 5-amino-1-(2-fluoro-4-(fluoromethoxy)-3-methoxyphenyl)-1H-imidazole-4-carbonyl chloride

5-amino-1-[2-fluoro-4-(fluoromethoxy)-3-methoxyphenyl]imidazole-4-carboxylic acid (25.0 mg, 0.08 mmol, 1.0 eq) was added to thionyl chloride (2 mL) at 20° C. under N₂, and the solution was stirred at 20° C. for 1 hour under N₂. The resulting solution was evaporated to dryness with methylene chloride to afford the title compound 5-amino-1-(2-fluoro-4-(fluoromethoxy)-3-methoxyphenyl)-1H-imidazole-4-carbonyl chloride (30 mg, 0.94 mmol, 94.3% yield), which was used without further purification. MS [M−Cl+OCH₃+H]⁺: 314.1.

### Step 10: tert-butyl (1R,5S,6r)-6-(4-(4-(5-amino-1-(2-fluoro-4-(fluoromethoxy)-3-methoxyphenyl)-1H-imidazole-4-carboxamido)-2-fluorobenzoyl)piperazine-1-carbonyl)-3-azabicyclo[3.1.0]hexane-3-carboxylate

To a solution of tert-butyl (1S,5R)-6-[4-(4-amino-2-fluorobenzoyl)piperazine-1-carbonyl]-3-azabicyclo[3.1.0]hexane-3-carboxylate (32.67 mg, 0.08 mmol, 0.8 eq) in methylene chloride (3 mL) was added pyridine (149.4 mg, 1.89 mmol, 20.0 eq) and 5-amino-1-[2-fluoro-4-(fluoromethoxy)-3-methoxyphenyl]imidazole-4-carbonyl chloride (30.0 mg, 0.09 mmol, 1.0 eq). The reaction mixture was stirred at 25° C. for 6 h and then concentrated to dryness. The residue was taken up in methylene chloride (20 mL) and washed with 2×20 mL water followed by 20 mL brine. The organics were then separated and dried (MgSO₄) before concentrating to dryness. The crude material was then purified by flash column chromatography eluting with with 5% MeOH in methylene chloride to afford tert-butyl (1S,5R)-6-[4-[4-[[5-amino-1-[2-fluoro-4-(fluoromethoxy)-3-methoxyphenyl]imidazole-4-carbonyl]amino]-2-fluorobenzoyl]piperazine-1-carbonyl]-3-azabicyclo[3.1.0]hexane-3-carboxylate (50.0 mg, 0.07 mmol, 61.7% yield). MS [M+H]⁺: 714.3.

### Step 11: 5-amino-1-[2-fluoro-4-(fluoromethoxy)-3-methoxyphenyl]-N-[3-fluoro-4-[4-[(1S,5R)-3-azabicyclo[3.1.0]hexane-6-carbonyl]piperazine-1-carbonyl]phenyl]imidazole-4-carboxamide; 1:1 2,2,2-trifluoroacetic acid

To a solution of tert-butyl (1S,5R)-6-[4-[4-[[5-amino-1-[2-fluoro-4-(fluoromethoxy)-3-methoxyphenyl]imidazole-4-carbonyl]amino]-2-fluorobenzoyl]piperazine-1-carbonyl]-3-azabicyclo[3.1.0]hexane-3-carboxylate (40.0 mg, 0.06 mmol, 1.0 eq) in methylene chloride (3 mL) was added trifluoroacetic acid (1.0 mL). The reaction mixture was stirred at 25° C. for 3 h and then concentrated to afford crude product, The crude product was purified by Prep-HPLC (Gemini C18 150×21.2 mm, 5 μm, MeCN-H₂O (0.1% TFA), 25-40) to afford 5-amino-1-[2-fluoro-4-(fluoromethoxy)-3-methoxyphenyl]-N-[3-fluoro-4-[4-[(1S,5R)-3-azabicyclo[3.1.0]hexane-6-carbonyl]piperazine-1-carbonyl]phenyl]imidazole-4-carboxamide; 1:1 2,2,2-trifluoroacetic acid (27.1 mg, 0.04 mmol, 78.6% yield). MS [M+H]⁺: 614.2.

### Compound 111

### N-(4-(4-((1R,5S,6r)-3-azabicyclo[3.1.0]hexane-6-carbonyl)piperazine-1-carbonyl)-3-fluorophenyl)-5-amino-1-(3-chloro-4-(fluoromethoxy)-2-methoxyphenyl)-1H-imidazole-4-carboxamide; 1:1 2,2,2-trifluoroacetic acid

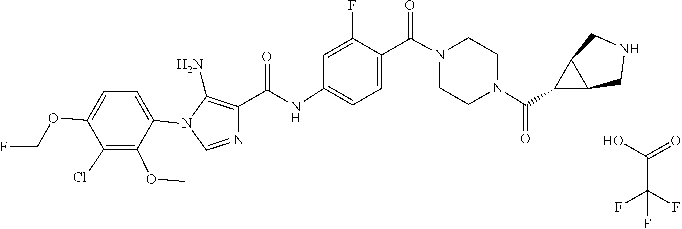 [SMILES: COc1c(-n2cnc(C(=O)Nc3ccc(C(=O)N4CCN(C(=O)[C@H]5[C@@H]6CNC[C@@H]65)CC4)c(F)c3)c2N)ccc(OCF)c1Cl ; O=C(O)C(F)(F)F]

### Step 1: 2-chloro-1-fluoro-3-methoxy-4-nitrobenzene

To a solution of 2-chloro-1,3-difluoro-4-nitrobenzene (2000.0 mg, 10.33 mmol, 1.0 eq) in methanol (2 mL) was added sodium methoxide (558.23 mg, 10.33 mmol, 1.0 eq). The reaction was stirred at 25° C. for 3 h. The resulting solution was concentrated under reduced pressure, and the residue was purified by flash column chromatography on silica gel (99:1 petroleum ether:EtOAc elution) to give 2-chloro-1-fluoro-3-methoxy-4-nitrobenzene (360.0 mg, 1.75 mmol, 17.0% yield).

¹H NMR (400 MHz, DMSO) δ 8.02 (dd, J=9.3, 5.7 Hz, 1H), 7.43 (dd, J=9.3, 8.3 Hz, 1H), 3.93 (s, 3H).

### Step 2: 2-chloro-3-methoxy-4-nitrophenol

A mixture of 2-chloro-1-fluoro-3-methoxy-4-nitrobenzene (310.0 mg, 1.51 mmol, 1.0 eq), potassium hydroxide (211.52 mg, 3.77 mmol, 2.5 eq) in DMSO (2.0 mL) was heated at 80° C. for 4 h. The resulting solution was diluted with 10 ml of water, then extracted with 3×10 mL of EtOAc. The organic layers were combined, washed with brine, dried and concentrated under reduced pressure. The residue was applied on a silica gel column and eluted with 90:10 petroleum ether:EtOAc to give 2-chloro-3-methoxy-4-nitrophenol (300.0 mg, 1.47 mmol, 85.0% yield). MS [M−H]⁻: 202.1

### Step 3: 2-chloro-1-(fluoromethoxy)-3-methoxy-4-nitrobenzene

To a solution of 2-chloro-3-methoxy-4-nitrophenol (330.0 mg, 1.62 mmol, 1.0 eq) in MeCN (5 mL) were added N,N-diisopropylethylamine (628.49 mg, 4.86 mmol, 3.0 eq) and fluoro(iodo)methane (388.87 mg, 2.43 mmol, 1.5 eq) under N₂ atmosphere. The vessel was sealed, and the mixture was stirred at 50° C. for 15 h. After concentration under reduced pressure, the residue was purified by flash column chromatography on silica gel (90:10 petroleum ether:EtOAc elution) to give 2-chloro-1-(fluoromethoxy)-3-methoxy-4-nitrobenzene (240.0 mg, 1.02 mmol, 62.2% yield). MS [(M+H]⁺: 236.0

### Step 4: 3-chloro-4-(fluoromethoxy)-2-methoxyaniline

A mixture of 2-chloro-1-(fluoromethoxy)-3-methoxy-4-nitrobenzene (190.0 mg, 0.81 mmol, 1.0 eq), iron (225.81 mg, 4.03 mmol, 5.0 eq) and ammonium chloride (215.69 mg, 4.03 mmol, 5.0 eq) in ethanol (5 mL) and water (2 mL) was stirred at 50° C. for 3 h. The solid was filtered off, and the crude was purified by flash column chromatography eluting with with 40% EtOAc in petroleum ether to give 3-chloro-4-(fluoromethoxy)-2-methoxyaniline (150.0 mg, 0.73 mmol, 76.0% yield). MS [(M+H]⁺: 206.1.

### Step 5: ethyl 5-amino-1-[3-chloro-4-(fluoromethoxy)-2-methoxyphenyl]imidazole-4-carboxylate

To a solution of ethyl 2-amino-2-cyanoacetate; 4-methylbenzene-1-sulfonic acid (832.59 mg, 2.77 mmol, 3.0 eq) in triethyl orthoformate (7.6 mL, 45 mmol, 49 eq) was added triethylamine (561.05 mg, 5.54 mmol, 6.0 eq). The resulting mixture was stirred at 83° C. for 2 h. Next, the solvent was removed under reduced pressure. The residue was diluted with MeCN (5 mL), and 3-chloro-4-(fluoromethoxy)-2-methoxyaniline (190.0 mg, 0.92 mmol, 1.0 eq) was added. The resulting mixture was stirred at 25° C. for 15 h. After concentration under reduced pressure, the crude material was diluted with EtOAc (20 mL), washed with H₂O (3×15 mL), dried over Na₂SO₄ and concentrated under reduced pressure. The residue was purified by flash column chromatography (45:55 petroleum ether:EtOAc elution) to give ethyl 5-amino-1-[3-chloro-4-(fluoromethoxy)-2-methoxyphenyl]imidazole-4-carboxylate (180.0 mg, 0.52 mmol, 55% yield). MS [(M+H]⁺: 344.1.

### Step 6: 5-amino-1-[3-chloro-4-(fluoromethoxy)-2-methoxyphenyl]imidazole-4-carboxylic acid

To a solution of potassium hydroxide (130.58 mg, 2.33 mmol, 5.0 eq) in isopropyl alcohol (2 mL) and water (1 mL) was added ethyl 5-amino-1-[3-chloro-4-(fluoromethoxy)-2-methoxyphenyl]imidazole-4-carboxylate (160.0 mg, 0.47 mmol, 1.0 eq). The resulting mixture was stirred at 70° C. for 6 h. The mixture was adjusted to pH 5-6 with 2N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure give 5-amino-1-[3-chloro-4-(fluoromethoxy)-2-methoxyphenyl]imidazole-4-carboxylic acid (100.0 mg, 0.32 mmol, 68.0% yield). MS [M+H]⁺: 316.0.

### Step 7: 5-amino-1-[3-chloro-4-(fluoromethoxy)-2-methoxyphenyl]imidazole-4-carbonyl chloride

A solution of 5-amino-1-[3-chloro-4-(fluoromethoxy)-2-methoxyphenyl]imidazole-4-carboxylic acid (80.0 mg, 0.25 mmol, 1.0 eq) in thionyl chloride (301.5 mg, 2.53 mmol, 10.0 eq) was stirred at 25° C. for 1 h. The resulting solution was concentrated under reduced pressure to give 5-amino-1-[3-chloro-4-(fluoromethoxy)-2-methoxyphenyl]imidazole-4-carbonyl chloride (80.0 mg, 0.24 mmol, 91.0% purity). MS [(M−Cl+OCH₃)+H]⁺: 330.0.

### Step 8: tert-butyl (1R,5S)-6-[4-[4-[[5-amino-1-[3-chloro-4-(fluoromethoxy)-2-methoxyphenyl]imidazole-4-carbonyl]amino]-2-fluorobenzoyl]piperazine-1-carbonyl]-3-azabicyclo[3.1.0]hexane-3-carboxylate

To a solution of tert-butyl (1R,5S)-6-[4-(4-amino-2-fluorobenzoyl)piperazine-1-carbonyl]-3-azabicyclo[3.1.0]hexane-3-carboxylate (103.55 mg, 0.24 mmol, 1.0 eq) and pyridine (0.19 mL, 2.39 mmol, 10.0 eq) in methylene chloride (2 mL) were added 5-amino-1-[3-chloro-4-(fluoromethoxy)-2-methoxyphenyl]imidazole-4-carbonyl chloride (80.0 mg, 0.24 mmol, 1.0 eq). The resulting mixture was stirred at 25° C. for 3 h. The reaction was concentrated to dryness and the residue was taken up in EtOAc (5 mL). The solution was washed with 2×3 mL water followed by 3 mL brine. The organics were then separated and dried (Na₂SO₄) before concentrating to dryness. The crude material was then purified by flash column chromatography with 90% EtOAc in petroleum ether elution to give tert-butyl (1R,5S)-6-[4-[4-[[5-amino-1-[3-chloro-4-(fluoromethoxy)-2-methoxyphenyl]imidazole-4-carbonyl]amino]-2-fluorobenzoyl]piperazine-1-carbonyl]-3-azabicyclo[3.1.0]hexane-3-carboxylate (130.0 mg, 0.18 mmol, 47.0% yield). MS [M+H]⁺: 730.3.

### Step 9: 5-amino-1-[3-chloro-4-(fluoromethoxy)-2-methoxyphenyl]-N-[3-fluoro-4-[4-[(1R,5S)-3-azabicyclo[3.1.0]hexane-6-carbonyl]piperazine-1-carbonyl]phenyl]imidazole-4-carboxamide; 1:1 2,2,2-trifluoroacetic acid

A solution of tert-butyl (1R,5S)-6-[4-[4-[[5-amino-1-[3-chloro-4-(fluoromethoxy)-2-methoxyphenyl]imidazole-4-carbonyl]amino]-2-fluorobenzoyl]piperazine-1-carbonyl]-3-azabicyclo[3.1.0]hexane-3-carboxylate (150.0 mg, 0.21 mmol, 1.0 eq) and trifluoroacetic acid (1.0 mL, 13 mmol, 63 eq) in methylene chloride (3 mL) was stirred at 25° C. After 1 h the reaction mixture was concentrated under reduced pressure. The residue was purified by Prep-HPLC (Gemini C18 150×21.2 mm, 5 μm, MeCN-H₂O (0.1% TFA)) to give 5-amino-1-[3-chloro-4-(fluoromethoxy)-2-methoxyphenyl]-N-[3-fluoro-4-[4-[(1R,5S)-3-azabicyclo[3.1.0]hexane-6-carbonyl]piperazine-1-carbonyl]phenyl]imidazole-4-carboxamide;1:1 2,2,2-trifluoroacetic acid (80.6 mg, 0.11 mmol, 51.7% yield). MS [M+H]⁺: 630.1.

### Compound 112

### N-(4-(4-((1R,5S,6r)-3-azabicyclo[3.1.0]hexane-6-carbonyl)piperazine-1-carbonyl)-3-fluorophenyl)-5-amino-1-(3-chloro-4-(fluoromethoxy)-2-methylphenyl)-1H-imidazole-4-carboxamide1:1 2,2,2-trifluoroacetate acid

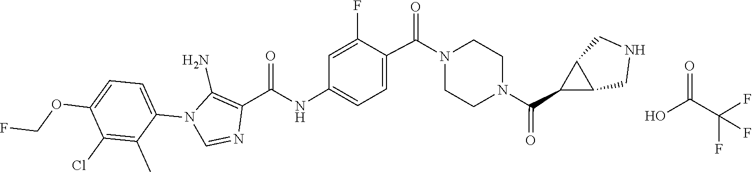 [SMILES: Cc1c(-n2cnc(C(=O)Nc3ccc(C(=O)N4CCN(C(=O)[C@H]5[C@@H]6CNC[C@@H]65)CC4)c(F)c3)c2N)ccc(OCF)c1Cl ; O=C(O)C(F)(F)F]

### Step 1: tert-butyl N-(4-bromo-3-chloro-2-methylphenyl) carbamate

A solution of 4-bromo-3-chloro-2-methyl-aniline (2.1 g, 9.5 mmol, 1.0 eq) and di-tert-butyl dicarbonate (3.12 g, 14.3 mmol, 1.5 eq) in 1,4-dioxane (20 mL) was stirred at 80° C. After 48 h, the reaction mixture was concentrated under reduced pressure. The residue was applied on a silica gel column and eluted with 30:1 petroleum ether:EtOAc to give tert-butyl N-(4-bromo-3-chloro-2-methylphenyl) carbamate (2.8 g, 8.73 mmol, 83.4% yield). MS [M−Boc+H]⁺: 266.0.

### Step 2: tert-butyl N-[3-chloro-2-methyl-4-(4,4,5,5-tetramethyl-1,3,2-dioxaborolan-2-yl)phenyl]carbamate

A solution of tert-butyl N-(4-bromo-3-chloro-2-methylphenyl) carbamate (680.0 mg, 2.12 mmol, 1.0 eq), bis(pinacolato)diboron (538.59 mg, 2.12 mmol, 1.0 eq), PdCl₂(dppf) (155.04 mg, 0.21 mmol, 0.1 eq) and potassium acetate (624.47 mg, 6.36 mmol, 3.0 eq) in 1,4-dioxane (10 mL) was stirred at 100° C. for 16 h under N₂. The resulting solution was concentrated under reduced pressure. The residue was applied on a silica gel column and eluted with 1:5 ethyl acetate:petroleum ether to give tert-butyl N-[3-chloro-2-methyl-4-(4,4,5,5-tetramethyl-1,3,2-dioxaborolan-2-yl)phenyl]carbamate (700.0 mg, 1.9 mmol, 79.9% yield). MS [M−Boc+H]⁺: 312.0.

### Step 3: tert-butyl N-(3-chloro-4-hydroxy-2-methylphenyl) carbamate

A solution of tert-butyl N-[3-chloro-2-methyl-4-(4,4,5,5-tetramethyl-1,3,2-dioxaborolan-2-yl)phenyl]carbamate (680.0 mg, 1.85 mmol, 1.0 eq) and hydrogen peroxide (314.5 mg, 9.25 mmol, 5.0 eq) in ethanol (5 mL) was stirred at 25° C. for 1 h. The residue was diluted with water, extracted with 3×20 ml of ethyl acetate. The organic layers were combined, washed with brine, dried and concentrated under reduced pressure. The residue was applied on a silica gel column and eluted with 1:5 ethyl acetate:petroleum ether to give tert-butyl N-(3-chloro-4-hydroxy-2-methylphenyl) carbamate (200.0 mg, 0.78 mmol, 21.4% yield). MS [M−Boc+H]⁺: 202.0.

### Step 4: tert-butyl N-[3-chloro-4-(fluoromethoxy)-2-methylphenyl]carbamate

To a solution of tert-butyl N-(3-chloro-4-hydroxy-2-methylphenyl) carbamate (190.0 mg, 0.74 mmol, 1.0 eq) in MeCN (5 mL) were added N, N-diisopropylethylamine (0.13 mL, 0.74 mmol, 1.0 eq) and fluoro(iodo)methane (175.84 mg, 1.11 mmol, 1.5 eq) under N₂ atmosphere. The vessel was sealed and the mixture was stirred at 50° C. for 15 h. After concentration under reduced pressure, the residue was purified by flash column chromatography on silica gel (95:5 petroleum ether:EtOAc) to afford tert-butyl N-[3-chloro-4-(fluoromethoxy)-2-methylphenyl]carbamate (155.0 mg, 0.53 mmol, 72.6% yield). MS [M−Boc+H]⁺: 234.0.

### Step 5: 3-chloro-4-(fluoromethoxy)-2-methyl-aniline

To a solution of tert-butyl N-[3-chloro-4-(fluoromethoxy)-2-methylphenyl]carbamate (150.0 mg, 0.52 mmol, 1.0 eq) in methanol (2 mL) was added hydrogen chloride (1.0 mL, 1 M in 1,4-dioxane). After stirring at 25° C. for 1 h, the resulting solution was concentrated under reduced pressure to afford 3-chloro-4-(fluoromethoxy)-2-methylaniline (95.0 mg, 0.5 mmol, 96.8% yield). MS [M+H]⁺: 190.0.

### Step 6: ethyl 5-amino-1-[3-chloro-4-(fluoromethoxy)-2-methylphenyl]imidazole-4-carboxylate

To a solution of ethyl 2-amino-2-cyanoacetate; 4-methylbenzene-1-sulfonic acid (285.11 mg, 0.95 mmol, 2.0 eq) in MeCN (3 mL) were added triethylamine (192.12 mg, 1.42 mmol, 4.0 eq) and triethyl orthoformate (703.43 mg, 4.74 mmol, 10.0 eq). After stirring the resulting mixture at 80° C. for 2 h. The solvent was removed under reduced pressure and the residue was diluted with MeCN (3 mL). Then 3-chloro-4-(fluoromethoxy)-2-methylaniline (90.0 mg, 0.47 mmol, 1.0 eq) was added and the resulting mixture was stirred at 40° C. for 15 h. After concentration under reduced pressure, the residue was purified by flash column chromatography on silica gel (1:3 petroleum ether:EtOAc elution) to provide ethyl ethyl 5-amino-1-[3-chloro-4-(fluoromethoxy)-2-methylphenyl]imidazole-4-carboxylate (60.0 mg, 0.18 mmol, 31.2% yield). MS [M+H]⁺: 328.0.

### Step 7: 5-amino-1-[3-chloro-4-(fluoromethoxy)-2-methylphenyl]imidazole-4-carboxylic acid

A solution of ethyl 5-amino-1-[3-chloro-4-(fluoromethoxy)-2-methylphenyl]imidazole-4-carboxylate (60.0 mg, 0.18 mmol, 1.0 eq), sodium hydroxide (73.22 mg, 1.83 mmol, 10.0 eq) in the isopropyl alcohol (3.0 mL) and water (1.0 mL) was stirred at 50° C. for 12 h. The residue was diluted with 5 mL of water, then adjusted to pH 5˜6 with HCl (2 M) and extracted with 3×5 mL of EtOAc. The organic layers were combined and concentrated under reduced pressure to give 5-amino-1-[3-chloro-4-(fluoromethoxy)-2-methylphenyl]imidazole-4-carboxylic acid (50.0 mg, 0.17 mmol, 82.9% yield). MS [M+H]⁺: 300.1

### Step 8: 5-amino-1-[3-chloro-4-(fluoromethoxy)-2-methylphenyl]imidazole-4-carbonyl chloride

A solution of 5-amino-1-[3-chloro-4-(fluoromethoxy)-2-methylphenyl]imidazole-4-carboxylic acid (30.0 mg, 0.1 mmol, 1.0 eq) in thionyl chloride (1.0 mL) was stirred at 25° C. for 1 h. The resulting solution was concentrated under reduced pressure to give 5-amino-1-[3-chloro-4-(fluoromethoxy)-2-methylphenyl]imidazole-4-carbonyl chloride (30.0 mg, 0.09 mmol, 94.2% yield). MS [M−Cl+OMe+H]⁺: 314.0.

### Step 9: tert-butyl (1R,5S)-6-[4-[4-[[5-amino-1-[3-chloro-4-(fluoromethoxy)-2-methylphenyl]imidazole-4-carbonyl]amino]-2-fluorobenzoyl]piperazine-1-carbonyl]-3-azabicyclo[3.1.0]hexane-3-carboxylate

A solution of tert-butyl (1R,5S)-6-[4-(4-amino-2-fluorobenzoyl)piperazine-1-carbonyl]-3-azabicyclo[3.1.0]hexane-3-carboxylate (40.78 mg, 0.09 mmol, 1.0 eq) and pyridine (74.59 mg, 0.94 mmol, 10.0 eq) in methylene chloride (2 mL) was stirred at 0° C. This was followed by the addition of 5-amino-1-[3-chloro-4-(fluoromethoxy)-2-methylphenyl]imidazole-4-carbonyl chloride (30.0 mg, 0.09 mmol, 1.0 eq) in methylene chloride (2 mL). The temperature was increased to 25° C. for 1 h. The resulting solution was concentrated under reduced pressure. The residue was applied on a silica gel column and eluted with 10:1 methylene chloride:MeOH to give tert-butyl (1R,5S)-6-[4-[4-[[5-amino-1-[3-chloro-4-(fluoromethoxy)-2-methylphenyl]imidazole-4-carbonyl]amino]-2-fluorobenzoyl]piperazine-1-carbonyl]-3-azabicyclo[3.1.0]hexane-3-carboxylate (50.0 mg, 0.07 mmol, 30.4% yield). MS [M−Boc+H]⁺: 658.1.

### Step 10: 5-amino-1-[3-chloro-4-(fluoromethoxy)-2-methylphenyl]-N-[3-fluoro-4-[4-[(1S,5R)-3-azoniabicyclo[3.1.0]hexane-6-carbonyl]piperazine-1-carbonyl]phenyl]imidazole-4-carboxamide; 1:1 2,2,2-trifluoroacetate acid

A solution of tert-butyl (1S,5R)-6-[4-[4-[[5-amino-1-[3-chloro-4-(fluoromethoxy)-2-methylphenyl]imidazole-4-carbonyl]amino]-2-fluorobenzoyl]piperazine-1-carbonyl]-3-azabicyclo[3.1.0]hexane-3-carboxylate (45.0 mg, 0.06 mmol, 1.0 eq), trifluoroacetic acid (1.0 mL) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40) to afford 5-amino-1-[3-chloro-4-(fluoromethoxy)-2-methylphenyl]-N-[3-fluoro-4-[4-[(1S,5R)-3-azoniabicyclo[3.1.0]hexane-6-carbonyl]piperazine-1-carbonyl]phenyl]imidazole-4-carboxamide; 1:1 2,2,2-trifluoroacetate acid (12.2 mg, 0.02 mmol, 25.7% yield). MS [M+H]⁺: 614.2.

### Compound 113

### 5-amino-1-[3-fluoro-4-(fluoromethoxy)-2-methylphenyl]-N-[3-fluoro-4-[4-[(1R,5S,6r)-3-azabicyclo[3.1.0]hexane-6-carbonyl]piperazine-1-carbonyl]phenyl]imidazole-4-carboxamide; 1:1 formic acid

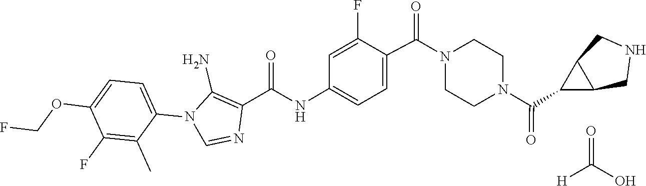

### Step 1: 2-fluoro-3-methyl-4-nitrophenol

A solution of 1,2-difluoro-3-methyl-4-nitrobenzene (4700.0 mg, 27.15 mmol, 1.0 eq) and potassium hydroxide (3808.0 mg, 67.87 mmol, 2.5 eq) in DMSO (10 mL) and water (30 mL) was stirred at 80° C. for 4 h. The mixture was adjusted to pH 5-6 with 2 N aqueous HCl and extracted with EtOAc (20 mL×3), the combined organic layer was washed with H₂O (3×15 mL), dried over anhydrous sodium sulfate and concentrated under reduced pressure give 2-fluoro-3-methyl-4-nitrophenol (3500.0 mg, 20.45 mmol, 75.3% yield). MS [M+H]⁺: 172.0.

### Step 2: 2-fluoro-1-(fluoromethoxy)-3-methyl-4-nitrobenzene

To a solution of 2-fluoro-3-methyl-4-nitrophenol (1000.0 mg, 5.84 mmol, 1.0 eq) in MeCN (20 mL) was added fluoroiodomethane (1869.11 mg, 11.69 mmol, 2.0 eq) and N,N-diisopropylethylamine (4.07 mL, 23.37 mmol, 4.0 eq). The reaction was stirred at 50° C. After 2 h, the reaction was concentrated to dryness and the residue was taken up in EtOAc (50 mL). The organic solution was washed with 20 mL water followed by 10 mL brine. The organics were then dried (Na₂SO₄) before concentration. The crude was then purified by flash column chromatography eluting with 20% EtOAc in petroleum ether elution to give 2-fluoro-1-(fluoromethoxy)-3-methyl-4-nitrobenzene (950.0 mg, 4.68 mmol, 80.0% yield). MS [M+H]⁺: 204.1.

### Step 3: 3-fluoro-4-(fluoromethoxy)-2-methylaniline

A solution of 2-fluoro-1-(fluoromethoxy)-3-methyl-4-nitrobenzene (1160.0 mg, 5.71 mmol, 1.0 eq), iron (1594.47 mg, 28.55 mmol, 5.0 eq) and ammonium chloride (1527.23 mg, 28.55 mmol, 5.0 eq) in ethanol (10 mL) and water (2 mL) was stirred at 50° C. for 2 h. The solid was filtered off. The crude was then purified by flash column chromatography eluting with 50% EtOAc in petroleum ether to give the 3-fluoro-4-(fluoromethoxy)-2-methylaniline (840.0 mg, 4.85 mmol, 80.6% yield). MS [M+H]⁺: 174.1.

### Step 4: ethyl 5-amino-1-[3-fluoro-4-(fluoromethoxy)-2-methylphenyl]imidazole-4-carboxylate

To a solution of ethyl 2-amino-2-cyanoacetate; 4-methylbenzene-1-sulfonic acid (693.76 mg, 2.31 mmol, 2.0 eq) in MeCN (4.0 mL) were added triethylamine (0.64 mL, 4.62 mmol, 4.0 eq) and triethyl orthoformate (5.11 mL, 30.6 mmol, 26.5 eq). The resulting mixture was stirred at 83° C. After for 2 h. The solvent was removed under reduced pressure. The residue was diluted with MeCN (4.0 mL), and then 3-fluoro-4-(fluoromethoxy)-2-methylaniline (200.0 mg, 1.16 mmol, 1.0 eq) was added. The reaction mixture was stirred at 20° C. for 15 h. After concentration under reduced pressure, the residue was purified by flash column chromatography on silica gel (55:45 petroleum ether:EtOAc elution) to afford ethyl 5-amino-1-[3-fluoro-4-(fluoromethoxy)-2-methylphenyl]imidazole-4-carboxylate (195.0 mg, 0.63 mmol, 50.2% yield). MS [M+H]⁺: 312.1.

### Step 5: 5-amino-1-[3-fluoro-4-(fluoromethoxy)-2-methylphenyl]imidazole-4-carboxylic acid

A solution of ethyl 5-amino-1-[3-fluoro-4-(fluoromethoxy)-2-methylphenyl]imidazole-4-carboxylate (230.0 mg, 0.74 mmol, 1.0 eq) and potassium hydroxide (165.82 mg, 2.96 mmol, 4.0 eq) in water (2.33 mL, 130 mmol, 175 eq) and ethanol (7 mL) was stirred at 50° C. for 12 h. The mixture was adjusted to pH 5-6 with 2 N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure give 5-amino-1-[3-fluoro-4-(fluoromethoxy)-2-methylphenyl]imidazole-4-carboxylic acid (150.0 mg, 0.53 mmol, 71.7% yield). MS [M+H]⁺: 284.1

### Step 6: 5-amino-1-[3-fluoro-4-(fluoromethoxy)-2-methylphenyl]imidazole-4-carbonyl chloride

A solution of 5-amino-1-[3-fluoro-4-(fluoromethoxy)-2-methylphenyl]imidazole-4-carboxylic acid (140.0 mg, 0.49 mmol, 1.0 eq) in thionyl chloride (588.07 mg, 4.94 mmol, 10.0 eq) was stirred at 25° C. for 1 h. The resulting solution was concentrated under reduced pressure to give 5-amino-1-[3-fluoro-4-(fluoromethoxy)-2-methylphenyl]imidazole-4-carbonyl chloride (120 mg, 0.40 mmol, 61.3% purity). MS [(M−Cl+OCH₃)+H]⁺: 298.1.

### Step 7: tert-butyl (1R,5S)-6-[4-[4-[[5-amino-1-[3-fluoro-4-(fluoromethoxy)-2-methylphenyl]imidazole-4-carbonyl]amino]-2-fluorobenzoyl]piperazine-1-carbonyl]-3-azabicyclo[3.1.0]hexane-3-carboxylate

To a solution of tert-butyl (1R,5S)-6-[4-(4-amino-2-fluorobenzoyl)piperazine-1-carbonyl]-3-azabicyclo[3.1.0]hexane-3-carboxylate (200.7 mg, 0.46 mmol, 1.0 eq) and pyridine (0.38 mL, 4.64 mmol, 10.0 eq) in methylene chloride (3.0 mL) were added 5-amino-1-[3-fluoro-4-(fluoromethoxy)-2-methylphenyl]imidazole-4-carbonyl chloride (140.0 mg, 0.46 mmol, 1.0 eq). The resulting mixture was stirred at 25° C. for 3 h. After concentration to dryness, the residue was taken up in EtOAc (30.0 mL) and the organics washed with 20 ml water followed by 10 mL brine. The organics were then separated and dried (Na₂SO₄) before concentrating to dryness. The crude was then purified by flash column chromatography eluting with with 90% EtOAc in petroleum ether to give tert-butyl (1R,5S)-6-[4-[4-[[5-amino-1-[3-fluoro-4-(fluoromethoxy)-2-methylphenyl]imidazole-4-carbonyl]amino]-2-fluorobenzoyl]piperazine-1-carbonyl]-3-azabicyclo[3.1.0]hexane-3-carboxylate (180.0 mg, 0.26 mmol, 53.4% yield). MS [(M−C₄H₈)+H]⁺: 642.1.

### Step 8: 5-amino-1-[3-fluoro-4-(fluoromethoxy)-2-methylphenyl]-N-[3-fluoro-4-[4-[(1R,5S)-3-azabicyclo[3.1.0]hexane-6-carbonyl]piperazine-1-carbonyl]phenyl]imidazole-4-carboxamide; 1:1 formic acid

To a solution of tert-butyl (1R,5S)-6-[4-[4-[[5-amino-1-[3-fluoro-4-(fluoromethoxy)-2-methylphenyl]imidazole-4-carbonyl]amino]-2-fluorobenzoyl]piperazine-1-carbonyl]-3-azabicyclo[3.1.0]hexane-3-carboxylate (180.0 mg, 0.26 mmol, 1.0 eq) and hydrogen chloride (4M in dioxane)(1.0 mL, 4.0 mmol, 15.5 eq) in methylene chloride (3 mL) was stirred at 25° C. for 1 h. The resulting solution was concentrated under reduced pressure. The residue was purified by Prep-HPLC (AQ.-C18, MeCN— H₂O (0.1% FA)) to provide 5-amino-1-[3-fluoro-4-(fluoromethoxy)-2-methylphenyl]-N-[3-fluoro-4-[4-[(1R,5S)-3-azabicyclo[3.1.0]hexane-6-carbonyl]piperazine-1-carbonyl]phenyl]imidazole-4-carboxamide; 1:1 formic acid (87.9 mg, 0.14 mmol, 52.7% yield). MS [M+H]⁺: 598.2.

### Compound 114

### N-(4-(4-((1R,5S,6r)-3-azabicyclo[3.1.0]hexane-6-carbonyl)piperazine-1-carbonyl)-3-fluorophenyl)-5-amino-1-(4-(fluoromethoxy)-2,3-dimethylphenyl)-1H-imidazole-4-carboxamide 2,2,2-trifluoroacetate; 1:1 trifluoroacetate

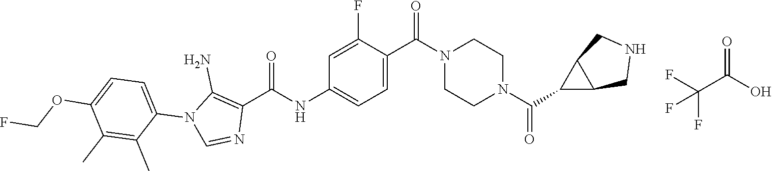

### Step 1: 1-(fluoromethoxy)-2,3-dimethyl-4-nitrobenzene

To a solution of 2,3-dimethyl-4-nitrophenol (1500.0 mg, 8.97 mmol, 1.0 eq) in MeCN (20 mL) was added N,N-diisopropylethylamine (3479.45 mg, 26.92 mmol, 3.0 eq) and fluoro(iodo)methane (2870.24 mg, 17.95 mmol, 2.0 eq). The reaction was stirred at 50° C. After 4 h, the reaction was concentrated to dryness and the residue was taken up in EtOAc (30 mL). The organics were washed with 2×30 mL water followed by 30 mL brine. The organics were then separated, dried (MgSO₄) and concentrated to dryness. The crude was then purified by flash column chromatography eluting with with 1:1 EtOAc:petroleum ether to afford 1-(fluoromethoxy)-2,3-dimethyl-4-nitrobenzene (1500.0 mg, 7.53 mmol, 62.7% yield). MS [M+H]⁺: 200.0.

### Step 2: 4-(fluoromethoxy)-2,3-dimethylaniline

A solution of 1-(fluoromethoxy)-2,3-dimethyl-4-nitrobenzene (1200.0 mg, 6.02 mmol, 1.0 eq) in methanol (15 mL) was added palladium 10% on carbon (320.57 mg, 3.01 mmol, 0.5 eq). The mixture was stirred 5 h at room temperature under an atmosphere of hydrogen (balloon). The mixture was filtered through diatomaceous earth. The filtrate was concentrated under reduced pressure to afford 4-(fluoromethoxy)-2,3-dimethylaniline (800.0 mg, 4.73 mmol, 76.9% yield), which was used in next step without further purification. MS [M+H]⁺: 170.1.

### Step 3: ethyl 5-amino-1-(4-(fluoromethoxy)-2,3-dimethylphenyl)-1H-imidazole-4-carboxylate

To a solution of ethyl 2-amino-2-cyanoacetate, 4-methylbenzenesulfonic acid (1:1)(4260.0 mg, 14.18 mmol, 3.0 eq) in triethyl orthoformate (34.33 mL, 205.19 mmol, 43.4 eq) was added triethylamine (2870.64 mg, 28.37 mmol, 6.0 eq). The resulting mixture was stirred at 80° C. for 2 h. The solvent was removed under reduced pressure and the residue was diluted with MeCN (20 mL). 4-(Fluoromethoxy)-2,3-dimethyl-aniline (800.0 mg, 4.73 mmol, 1.0 eq) was added, and the resulting mixture was stirred at 25° C. for 15 h. The reaction was then concentrated to dryness. The residue was taken up in EtOAc (30 mL) and the organics washed with 2×30 mL water followed by 30 mL brine. The organics were then separated and dried (MgSO₄) before concentrating to dryness. The crude was then purified by flash column chromatography eluting with with 3% MeOH in methylene chloride to afford 5-amino-1-[4-(fluoromethoxy)-2,3-dimethylphenyl]imidazole-4-carboxylate (890.0 mg, 2.9 mmol, 55.5% yield). MS [M+H]⁺: 308.1.

### Step 4: 5-amino-1-(4-(fluoromethoxy)-2,3-dimethylphenyl)-1H-imidazole-4-carboxylic acid

A solution of ethyl 5-amino-1-[4-(fluoromethoxy)-2,3-dimethylphenyl]imidazole-4-carboxylate (890.0 mg, 2.9 mmol, 1.0 eq) in methanol (10 mL) and water (1 mL) was lithium hydroxide (0.27 mL, 28.96 mmol, 10.0 eq) the reaction was stirred at 60° C. for 6 h, after which time solvent was evaporated. The residue was diluted with water (10 mL), and the solution was extracted with ethyl acetate (2×10 mL). The aqueous layer was acidified to pH 5 with HCl (2 M), and the product was collected by filtration to afford 5-amino-1-[4-(fluoromethoxy)-2,3-dimethylphenyl]imidazole-4-carboxylic acid (475.0 mg, 1.7 mmol, 46.4% yield). MS [M+H]⁺: 280.1.

### Step 5: 5-amino-1-(4-(fluoromethoxy)-2,3-dimethylphenyl)-1H-imidazole-4-carbonyl chloride

To 5-amino-1-[4-(fluoromethoxy)-2,3-dimethylphenyl]imidazole-4-carboxylic acid (100.0 mg, 0.36 mmol, 1.0 eq) under N₂ was added thionyl chloride (3 mL) at 20° C., the solution was stirred at 20° C. for 1 hour under N₂. The resulting solution was evaporated to dryness with methylene chloride to afford the title compound 5-amino-1-[4-(fluoromethoxy)-2,3-dimethylphenyl]imidazole-4-carbonyl chloride (100.0 mg, 0.34 mmol, 67.3% yield), which was used without further purification. MS [M-Cl+OCH₃+H]⁺: 294.1.

### Step 6: tert-butyl (1R,5S)-6-[4-[5-amino-1-[4-(fluoromethoxy)-2,3-dimethylphenyl]imidazole-4-carbonyl]amino]-2-fluorobenzoyl]piperazine-1-carbonyl]-3-azabicyclo[3.1.0]hexane-3-carboxylate

To a solution of tert-butyl (1R,5S)-6-[4-(4-amino-2-fluorobenzoyl)piperazine-1-carbonyl]-3-azabicyclo[3.1.0]hexane-3-carboxylate (145.27 mg, 0.34 mmol, 1.0 eq) in methylene chloride (5 mL) were added pyridine (0.27 mL, 3.36 mmol, 10.0 eq) and 5-amino-1-[4-(fluoromethoxy)-2,3-dimethylphenyl]imidazole-4-carbonyl chloride (100.0 mg, 0.34 mmol, 1.0 eq) at 0° C., then the mixture was allowed warm to 25° C. and stirred for 5 h. The reaction was concentrated to dryness and the residue was taken up in methylene chloride (20 mL) and the organics washed with 2×20 ml water and 20 mL brine. The organics were then separated and dried (MgSO₄) before concentrating to dryness. The crude was then purified by flash column chromatography eluting with EtOAc to afford tert-butyl (1R,5S)-6-[4-[5-amino-1-[4-(fluoromethoxy)-2,3-dimethylphenyl]imidazole-4-carbonyl]amino]-2-fluorobenzoyl]piperazine-1-carbonyl]-3-azabicyclo[3.1.0]hexane-3-carboxylate (150.0 mg, 0.22 mmol, 46.1% yield). MS [M+H]⁺: 694.2.

### Step 7: N-(4-(4-((1R,5S,6r)-3-azabicyclo[3.1.0]hexane-6-carbonyl)piperazine-1-carbonyl)-3-fluorophenyl)-5-amino-1-(4-(fluoromethoxy)-2,3-dimethylphenyl)-1H-imidazole-4-carboxamide 1:1 2,2,2-trifluoroacetate

To a solution of tert-butyl (1R,5S)-6-[4-[4-[[5-amino-1-[4-(fluoromethoxy)-2,3-dimethylphenyl]imidazole-4-carbonyl]amino]-2-fluorobenzoyl]piperazine-1-carbonyl]-3-azabicyclo[3.1.0]hexane-3-carboxylate (140.0 mg, 0.2 mmol, 1.0 eq) in methylene chloride (3 mL) was added trifluoroacetic acid (1.0 mL). The reaction was stirred at 25° C. for 3 h. It was then concentrated to dryness to afford crude product. The crude was purified by HPLC (Gemini-C18 150×21.2 mm, 5 μm, MeCN-H₂O (0.1% TFA), 20-60) to afford 5-amino-1-[4-(fluoromethoxy)-2,3-dimethylphenyl]-N-[3-fluoro-4-[4-[(1R,5S)-3-azabicyclo[3.1.0]hexane-6-carbonyl]piperazine-1-carbonyl]phenyl]imidazole-4-carboxamide; 1:1 2,2,2-trifluoroacetic acid (61.1 mg, 0.09 mmol, 50.9% yield). MS [M+H]⁺: 594.2.

### Compound 115

### N-(4-(4-((1R,5S,6r)-3-azabicyclo[3.1.0]hexane-6-carbonyl)piperazine-1-carbonyl)-3-fluorophenyl)-5-amino-1-(2-fluoro-4-(fluoromethoxy)-3-methylphenyl)-1H-imidazole-4-carboxamide; 1:1 2,2,2-trifluoroacetate acid

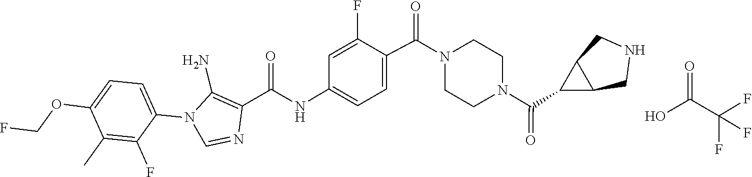 [SMILES: Cc1c(OCF)ccc(-n2cnc(C(=O)Nc3ccc(C(=O)N4CCN(C(=O)[C@H]5[C@@H]6CNC[C@@H]65)CC4)c(F)c3)c2N)c1F ; O=C(O)C(F)(F)F]

### Step 1: 3-fluoro-1-methoxy-2-methyl-4-nitrobenzene

A solution of 1,3-difluoro-2-methyl-4-nitrobenzene (2.0 g, 11.55 mmol, 1.0 eq) and sodium carbonate (1224.45 mg, 11.55 mmol, 1.0 eq) in methanol (30 mL) was stirred at 60° C. for 48 h. The resulting solution was concentrated under reduced pressure. The residue was applied on a silica gel column and eluted with 30:1 petroleum ether:EtOAc to give 3-fluoro-1-methoxy-2-methyl-4-nitrobenzene (1.0 g, 5.4 mmol, 46.8% yield). MS [M+H]⁺: 186.0.

### Step 2: 3-fluoro-2-methyl-4-nitrophenol

A solution of 3-fluoro-1-methoxy-2-methyl-4-nitrobenzene (900.0 mg, 4.86 mmol, 1.0 eq) and boron tribromide (8 mL) in methylene chloride 8 ml) was stirred at 80° C. After 16 h, the reaction mixture was cooled to room temperature, quenched with water (20 ml), and extracted with methylene chloride (20 ml). The combined extracts were washed with water (20 ml) followed by brine (40 ml), dried (Na₂SO₄), and concentrated to give 2,3-dichloro-4-nitrophenol (300 mg, 1.75 mmol, 36.1% yield). MS [M−H]: 170.0.

### Step 3: 3-fluoro-1-(fluoromethoxy)-2-methyl-4-nitrobenzene

To a solution of 3-fluoro-2-methyl-4-nitrophenol (290.0 mg, 1.69 mmol, 1.0 eq) in MeCN (20 mL) were added N, N-diisopropylethylamine (0.3 mL, 1.7 mmol, 1.0 eq) and fluoro(iodo)methane (404.17 mg, 2.54 mmol, 1.5 eq) under N₂ atmosphere. The vessel was sealed and the mixture was stirred at 50° C. for 15 h. After concentration under reduced pressure, the residue was purified by flash column chromatography on silica gel (95:5 petroleum ether:EtOAc) to give 3-fluoro-1-(fluoromethoxy)-2-methyl-4-nitrobenzene (220.0 mg, 1.08 mmol, 61.4% yield). MS [M+H]⁺: 204.0.

### Step 4: 2-fluoro-4-(fluoromethoxy)-3-methyl-aniline

A solution of 3-fluoro-1-(fluoromethoxy)-2-methyl-4-nitrobenzene (210.0 mg, 1.03 mmol, 1.0 eq), ammonium chloride (276.48 mg, 5.17 mmol, 5.0 eq) and iron dust (289.46 mg, 5.17 mmol, 5.0 eq) in ethanol (2 mL) water (2 mL) was heated at 50° C. was reacted for 2 h. The solid was filtered out. The filtrate was concentrated under reduced pressure. The residue was diluted with water, then extracted with 2×10 ml of ethyl acetate. The organic layers were combined, washed with brine, dried and concentrated under reduced pressure to afford 2-fluoro-4-(fluoromethoxy)-3-methyl-aniline (170.0 mg, 0.98 mmol, 95.0% yield). MS [M+H]⁺: 174.0.

### Step 5: ethyl 5-amino-1-[2-fluoro-4-(fluoromethoxy)-3-methylphenyl]imidazole-4-carboxylate

To a solution of ethyl 2-amino-2-cyanoacetate; 4-methylbenzene-1-sulfonic acid (555.01 mg, 1.85 mmol, 2.0 eq) in MeCN (5 mL) were added triethylamine (0.52 mL, 3.7 mmol, 4.0 eq) and triethyl orthoformate (1369.37 mg, 9.24 mmol, 10.0 eq). The resulting mixture was stirred at 80° C. for 2 h. The solvent was removed under reduced pressure. The residue was diluted with MeCN (5 mL), then 2-fluoro-4-(fluoromethoxy)-3-methyl-aniline (160.0 mg, 0.92 mmol, 1.0 eq) was added and the resulting mixture was stirred at 40° C. for 15 h. After concentration under reduced pressure, the residue was purified by flash column chromatography on silica gel (1:3 petroleum ether:EtOAc) to give ethyl 5-amino-1-[2-fluoro-4-(fluoromethoxy)-3-methylphenyl]imidazole-4-carboxylate (140.0 mg, 0.45 mmol, 46.2% yield). MS [M+H]⁺: 312.0.

### Step 6: 5-amino-1-[2-fluoro-4-(fluoromethoxy)-3-methylphenyl]imidazole-4-carboxylic acid

A solution of ethyl 5-amino-1-[2-fluoro-4-(fluoromethoxy)-3-methylphenyl]imidazole-4-carboxylate (130.0 mg, 0.42 mmol, 1.0 eq), lithium hydroxide (167.0 mg, 4.18 mmol, 10.0 eq) in methanol (3 mL) and water (1 mL) was stirred at 50° C. for 12 h. The residue was diluted with 10 ml of water, then adjusted to pH 5˜6 with HCl (2 M) and extracted with 3×10 mL of EtOAc. The organic layers were combined and concentrated under reduced pressure to give 5-amino-1-[2-fluoro-4-(fluoromethoxy)-3-methylphenyl]imidazole-4-carboxylic acid (80.0 mg, 0.28 mmol, 58.2% yield). MS [M+H]⁺: 284.1

### Step 7: 5-amino-1-[2-fluoro-4-(fluoromethoxy)-3-methylphenyl]imidazole-4-carbonyl chloride

A solution of 5-amino-1-[2-fluoro-4-(fluoromethoxy)-3-methylphenyl]imidazole-4-carboxylic acid (60.0 mg, 0.21 mmol, 1.0 eq) in thionyl chloride (2.0 mL) was stirred at 25° C. for 1 h. The resulting solution was concentrated under reduced pressure to give 5-amino-1-[2-fluoro-4-(fluoromethoxy)-3-methylphenyl]imidazole-4-carbonyl chloride (50.0 mg, 0.17 mmol, 72.8% yield). MS [M−Cl+OMe+H]⁺: 298.0

### Step 8: tert-butyl (1R,5S)-6-[4-[4-[[5-amino-1-[2-fluoro-4-(fluoromethoxy)-3-methylphenyl]imidazole-4-carbonyl]amino]-2-fluorobenzoyl]piperazine-1-carbonyl]-3-azabicyclo[3.1.0]hexane-3-carboxylate

A solution of tert-butyl (1R,5S)-6-[4-(4-amino-2-fluorobenzoyl)piperazine-1-carbonyl]-3-azabicyclo[3.1.0]hexane-3-carboxylate (78.85 mg, 0.18 mmol, 1.1 eq) and pyridine (0.03 mL, 0.33 mmol, 2.0 eq) in methylene chloride (2 mL) was stirred at 0° C., this was followed by the addition of 5-amino-1-[2-fluoro-4-(fluoromethoxy)-3-methylphenyl]imidazole-4-carbonyl chloride (50.0 mg, 0.17 mmol, 1.0 eq) in methylene chloride (2 mL). The temperature was increased to 25° C. for 1 h. The resulting solution was concentrated under reduced pressure. The residue was adsorbed on a silica gel column and eluted with 10:1 methylene chloride:MeOH to give tert-butyl (1R,5S)-6-[4-[4-[[5-amino-1-[2-fluoro-4-(fluoromethoxy)-3-methylphenyl]imidazole-4-carbonyl]amino]-2-fluorobenzoyl]piperazine-1-carbonyl]-3-azabicyclo[3.1.0]hexane-3-carboxylate (80.0 mg, 0.11 mmol, 45.0% yield). MS [M+H]⁺: 698.1.

### Step 9: 5-amino-N-[4-[4-[(1R,5S)-3-azoniabicyclo[3.1.0]hexane-6-carbonyl]piperazine-1-carbonyl]-3-fluorophenyl]-1-[2-fluoro-4-(fluoromethoxy)-3-methylphenyl]imidazole-4-carboxamide; 1:1 2,2,2-trifluoroacetate acid

To a solution of tert-butyl (1R,5S)-6-[4-[4-[[5-amino-1-[2-fluoro-4-(fluoromethoxy)-3-methylphenyl]imidazole-4-carbonyl]amino]-2-fluorobenzoyl]piperazine-1-carbonyl]-3-azabicyclo[3.1.0]hexane-3-carboxylate (70.0 mg, 0.1 mmol, 1.0 eq) in methylene chloride (4 mL) was added trifluoroacetic acid (1.0 mL). It was then stirred at 25° C. for 1 h. The resulting solution was concentrated under reduced pressure. The crude product was purified by Prep-HPLC (Gemini C18 150×21.2 mm, 5 μm, MeCN— H₂O (0.1% TFA), 15-40) to afford 5-amino-N-[4-[4-[(1R,5S)-3-azoniabicyclo[3.1.0]hexane-6-carbonyl]piperazine-1-carbonyl]-3-fluoro-phenyl]-1-[2-fluoro-4-(fluoromethoxy)-3-methylphenyl]imidazole-4-carboxamide; 1:1 2,2,2-trifluoroacetate acid (34.6 mg, 0.05 mmol, 48.4% yield). MS [M+H]⁺: 598.2.

### Compound 116

### 5-amino-N-[4-[4-[(1R,5S,6r)-3-azabicyclo[3.1.0]hexane-6-carbonyl]piperazine-1-carbonyl]-3-fluoro-phenyl]-1-[2-chloro-4-(fluoromethoxy)-3-methylphenyl]imidazole-4-carboxamide; 1:1 2,2,2-trifluoroacetic acid

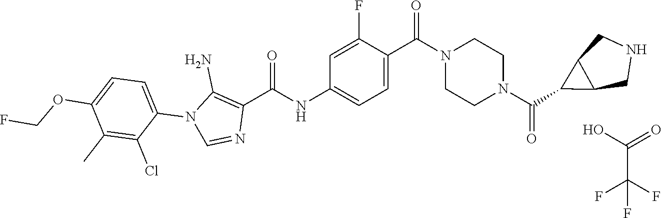

### Step 1: 3-chloro-1-(fluoromethoxy)-2-methyl-4-nitrobenzene

To a solution of 3-chloro-2-methyl-4-nitrophenol (776.0 mg, 4.14 mmol, 1.0 eq) in MeCN (10 mL) were added N,N-diisopropylethylamine (1603.96 mg, 12.41 mmol, 3.0 eq) and fluoro(iodo)methane (992.42 mg, 6.21 mmol, 1.5 eq) under N₂ atmosphere. The vessel was sealed, and the mixture was stirred at 50° C. for 15 h. After concentration under reduced pressure, the residue was purified by flash column chromatography on silica gel (90:10 petroleum ether:EtOAc) to give 3-chloro-1-(fluoromethoxy)-2-methyl-4-nitrobenzene (610.0 mg, 2.78 mmol, 65.8% yield). MS [M+H]⁺: 219.9.

### Step 2: 2-chloro-4-(fluoromethoxy)-3-methyl-aniline

A solution of 3-chloro-1-(fluoromethoxy)-2-methyl-4-nitrobenzene (610.0 mg, 2.78 mmol, 1.0 eq), ammonium chloride (742.92 mg, 13.89 mmol, 5.0 eq) and iron powder (777.78 mg, 13.89 mmol, 5.0 eq) in ethanol (5 mL) and water (5 mL) was heated at 50° C., then stirred for 6 h. The reaction mixture was quenched by the addition of the saturated aqueous NaCl. After quenching the reaction, the reaction mixture was poured into separatory funnel and separated. The residue was partitioned between ethyl acetate (50 mL). The organic layer was washed with water, dried (Na₂SO₄) and evaporated to dryness to afford 2-chloro-4-(fluoromethoxy)-3-methyl-aniline (400.0 mg, 2.11 mmol, 71.4% yield). MS [M+H]⁺: 190.0.

### Step 3: ethyl 5-amino-1-[3-chloro-4-(fluoromethoxy)-2-methylphenyl]imidazole-4-carboxylate

To a solution of ethyl 2-amino-2-cyanoacetate; 4-methylbenzene-1-sulfonic acid (633.57 mg, 2.11 mmol, 2.0 eq) in MeCN (10 mL) were added triethylamine (0.59 mL, 4.22 mmol, 4.0 eq) and triethyl orthoformate (1563.21 mg, 10.55 mmol, 10.0 eq). The resulting mixture was stirred at 80° C. for 2 h. The solvent was removed under reduced pressure and the residue was diluted with MeCN (10 mL). 2-Chloro-4-(fluoromethoxy)-3-methyl-aniline (200.0 mg, 1.05 mmol, 1.0 eq) was then added, and the resulting mixture was stirred at 25° C. for 15 h. After concentration under reduced pressure, the residue was purified by flash column chromatography on silica gel (1:3 petroleum ether:EtOAc) to give ethyl 5-amino-1-[3-chloro-4-(fluoromethoxy)-2-methylphenyl]imidazole-4-carboxylate (120.0 mg, 0.37 mmol, 34.7% yield). MS [M+H]⁺: 328.0.

### Step 4: 5-amino-1-[2-chloro-4-(fluoromethoxy)-3-methylphenyl]imidazole-4-carboxylic acid

To a solution of ethyl 5-amino-1-[2-chloro-4-(fluoromethoxy)-3-methylphenyl]imidazole-4-carboxylate (60.0 mg, 0.18 mmol, 1.0 eq) in methanol (4 mL) were added potassium hydroxide (61.62 mg, 1.1 mmol, 6.0 eq) and water (1.5 mL) under N₂. The resulting mixture was stirred at 50° C. for 5 h. The methanol was removed under reduced pressure, and diluted with water (3 mL), then acidified with HCl (1N), adjusted the pH of the reaction solution to 6-7. The mixture was filtered to afford 5-amino-1-[2-chloro-4-(fluoromethoxy)-3-methylphenyl]imidazole-4-carboxylic acid (90.0 mg, 0.3 mmol, 65.6% yield). The crude product was used directly without further purification. MS [M+H]⁺: 314.0.

### Step 5: 5-amino-1-[2-chloro-4-(fluoromethoxy)-3-methylphenyl]imidazole-4-carbonyl chloride

A solution of 5-amino-1-[2-chloro-4-(fluoromethoxy)-3-methylphenyl]imidazole-4-carboxylic acid (60.0 mg, 0.2 mmol, 1.0 eq) in thionyl chloride (6.0 mL, 82.61 mmol, 412.62 eq) was stirred at 25° C. for 1.5 h. The resulting solution was concentrated under reduced pressure to give 5-amino-1-[2-chloro-4-(fluoromethoxy)-3-methyl-phenyl]imidazole-4-carbonyl chloride (60.0 mg, 0.19 mmol, 91.2% yield). MS [(M−Cl+OCH₃)+H]⁺: 314.0.

### Step 6: tert-butyl (1R,5S)-6-[4-[4-[[5-amino-1-[2-chloro-4-(fluoromethoxy)-3-methylphenyl]imidazole-4-carbonyl]amino]-2-fluorobenzoyl]piperazine-1-carbonyl]-3-azabicyclo[3.1.0]hexane-3-carboxylate

A solution of tert-butyl (1R,5S)-6-[4-(4-amino-2-fluorobenzoyl)piperazine-1-carbonyl]-3-azabicyclo[3.1.0]hexane-3-carboxylate (36.71 mg, 0.08 mmol, 1.0 eq) and pyridine (0.1 mL, 1.21 mmol, 14.3 eq) in methylene chloride (2 mL) was stirred at 0° C. Next 5-amino-1-[2-chloro-4-(fluoromethoxy)-3-methylphenyl]imidazole-4-carbonyl chloride (90.0 mg, 0.08 mmol, 1.0 eq) in methylene chloride (2 mL) was added, and the temperature was increased to 25° C. for 2 h. The solvent was then removed under reduced pressure and the residue was purified by flash column chromatography (90:10 methylene chloride:MeOH elution) to give tert-butyl (1R,5S)-6-[4-[4-[[5-amino-1-[2-chloro-4-(fluoromethoxy)-3-methylphenyl]imidazole-4-carbonyl]amino]-2-fluorobenzoyl]piperazine-1-carbonyl]-3-azabicyclo[3.1.0]hexane-3-carboxylate (40.0 mg, 0.06 mmol, 42.9% yield). MS [M+H]⁺: 714.3.

### Step 7: 5-amino-N-[4-[4-[(1R,5S)-3-azabicyclo[3.1.0]hexane-6-carbonyl]piperazine-1-carbonyl]-3-fluoro-phenyl]-1-[2-chloro-4-(fluoromethoxy)-3-methylphenyl]imidazole-4-carboxamide; 1:1 2,2,2-trifluoroacetic acid

To a solution of tert-butyl (1R,5S)-6-[4-[4-[[5-amino-1-[2-chloro-4-(fluoromethoxy)-3-methylphenyl]imidazole-4-carbonyl]amino]-2-fluorobenzoyl]piperazine-1-carbonyl]-3-azabicyclo[3.1.0]hexane-3-carboxylate (30.0 mg, 0.03 mmol, 1.0 eq) in methylene chloride (2 mL) was added trifluoroacetic acid (0.3 mL, 4.0 mmol, 150 eq) under N₂ atmosphere. The resulting mixture was stirred at 25° C. for 2 h. Next the solvent was removed under reduced pressure, and the residue was purified by prep-HPLC [Gemini-C18, 150×21.2 mm, 5 μm, MeCN-H₂O (0.1% TFA), 15%˜40%] to give 5-amino-N-[4-[4-[(1R,5S)-3-azabicyclo[3.1.0]hexane-6-carbonyl]piperazine-1-carbonyl]-3-fluoro-phenyl]-1-[2-chloro-4-(fluoromethoxy)-3-methylphenyl]imidazole-4-carboxamide; 1:1 2,2,2-trifluoroacetic acid (6.5 mg, 0.01 mmol, 32.4% yield). MS [M+H]⁺: 614.4.

### Compound 117

### 5-amino-N-[4-[4-[(1R,5S,6r)-3-azabicyclo[3.1.0]hexane-6-carbonyl]piperazine-1-carbonyl]-3-fluoro-phenyl]-1-[2,3-dichloro-4-(fluoromethoxy)phenyl]imidazole-4-carboxamide; 1:1 2,2,2-trifluoroacetic acid

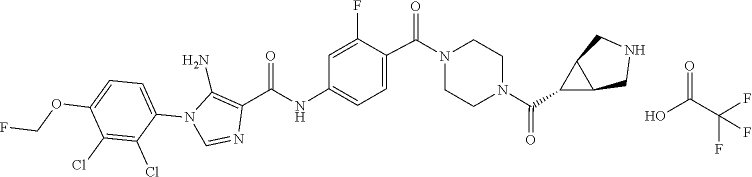 [SMILES: Nc1c(C(=O)Nc2ccc(C(=O)N3CCN(C(=O)[C@H]4[C@@H]5CNC[C@@H]54)CC3)c(F)c2)ncn1-c1ccc(OCF)c(Cl)c1Cl ; O=C(O)C(F)(F)F]

### Step 1: 2,3-dichloro-1-(fluoromethoxy)-4-nitrobenzene

To a solution of 2,3-dichloro-4-nitrophenol (1.1 g, 5.29 mmol, 1.0 eq) in MeCN (20 mL) were added N,N-diisopropylethylamine (2.05 g, 15.9 mmol, 3.0 eq) and fluoro(iodo)methane (1.27 g, 7.93 mmol, 1.5 eq) under N₂ atmosphere. The vessel was sealed, and the mixture was stirred at 50° C. for 15 h. After concentration under reduced pressure, the residue was purified by flash column chromatography on silica gel (petroleum ether:EtOAc=95:5) to give 2,3-dichloro-1-(fluoromethoxy)-4-nitrobenzene (1.1 g, 4.58 mmol, 86.7% yield).

### Step 2: 2,3-dichloro-4-(fluoromethoxy)aniline

To a solution of 2,3-dichloro-1-(fluoromethoxy)-4-nitrobenzene (1.0 g, 4.2 mmol, 1.0 eq) in ethanol (10 mL)/water (10 mL) added ammonium chloride (1.11 g, 20.8 mmol, 5.0 eq) and Fe (1.17 g, 20.8 mmol, 5.0 eq), then heated to 60° C. and stirred for 4 h. The reaction mixture was quenched by the addition of a saturated aqueous NaCl solution. After quenching, the reaction mixture was poured into a separatory funnel and partitioned. The residue was extracted with ethyl acetate (100 mL). The organic layer was washed with water, dried (Na₂SO₄) and evaporated to dryness to afford 2,3-dichloro-4-(fluoromethoxy)aniline (0.8 g, 3.8 mmol, 83% yield). MS [M+H]⁺: 210.0.

### Step 3: ethyl 5-amino-1-[2,3-dichloro-4-(fluoromethoxy)phenyl]imidazole-4-carboxylate

To a solution of ethyl 2-amino-2-cyanoacetate, 4-methylbenzenesulfonic acid (1:1)(3002.87 mg, 10.0 mmol, 3.0 eq) in triethyl orthoformate (4939.29 mg, 33.33 mmol, 10.0 eq) was added triethylamine (2.79 mL, 20.0 mmol, 6.0 eq). The resulting mixture was stirred at 80° C. for 2 h. The solvent was removed under reduced pressure and the residue was diluted with MeCN (10 mL), then 2,3-dichloro-4-(fluoromethoxy)aniline (700.0 mg, 3.33 mmol, 1.0 eq) was added and the resulting mixture was stirred at 40° C. for 15 h. After concentration under reduced pressure, the residue was purified by flash column chromatography on silica gel (1:3 petroleum ether:EtOAc) to give ethyl 5-amino-1-[2,3-dichloro-4-(fluoromethoxy)phenyl]imidazole-4-carboxylate (800.0 mg, 2.3 mmol, 68.9% yield). MS [M+H]⁺: 348.0.

### Step 4: 5-amino-1-[2,3-dichloro-4-(fluoromethoxy)phenyl]imidazole-4-carboxylic acid

A solution of ethyl 5-amino-1-[2,3-dichloro-4-(fluoromethoxy)phenyl]imidazole-4-carboxylate (100.0 mg, 0.29 mmol, 1.0 eq), potassium hydroxide (64.46 mg, 1.15 mmol, 4.0 eq) in the ethanol (3 mL) and water (1 mL) was stirred at 70° C. for 6 h. The residue was diluted with 100 ml of water, then adjusted to pH 6˜7 with HCl (2 M) and extracted with 100 mL of EtOAc. The organic layers were combined and concentrated under reduced pressure to give 5-amino-1-[2,3-dichloro-4-(fluoromethoxy)phenyl]imidazole-4-carboxylic acid (70.0 mg, 0.22 mmol, 76.1% yield). MS [M+H]⁺: 319.9.

### Step 5: 5-amino-1-[2,3-dichloro-4-(fluoromethoxy)phenyl]imidazole-4-carbonyl chloride

A solution of 5-amino-1-[2,3-dichloro-4-(fluoromethoxy)phenyl]imidazole-4-carboxylic acid (70.0 mg, 0.22 mmol, 1.0 eq) in thionyl chloride (1.79 mL, 24.7 mmol, 113.0 eq) was stirred at 25° C. for 1 h. The resulting solution was concentrated under reduced pressure to give 5-amino-1-[2,3-dichloro-4-(fluoromethoxy)phenyl]imidazole-4-carbonyl chloride (70.0 mg, 0.21 mmol, 94.6% yield). MS [M-Cl+OMe+H]⁺: 351.0.

### Step 6: tert-butyl (1R,5S)-6-[4-[4-[[5-amino-1-[2,3-dichloro-4-(fluoromethoxy)phenyl]imidazole-4-carbonyl]amino]-2-fluorobenzoyl]piperazine-1-carbonyl]-3-azabicyclo[3.1.0]hexane-3-carboxylate

A solution of tert-butyl (1R,5S)-6-[4-(4-amino-2-fluorobenzoyl)piperazine-1-carbonyl]-3-azabicyclo[3.1.0]hexane-3-carboxylate (89.42 mg, 0.21 mmol, 1.0 eq) and pyridine (0.05 mL, 0.62 mmol, 3.0 eq) in methylene chloride (2 mL) was stirred at 25° C. This was followed by the addition of 5-amino-1-[2,3-dichloro-4-(fluoromethoxy)phenyl]imidazole-4-carbonyl chloride (70.0 mg, 0.21 mmol, 1.0 eq) in methylene chloride (2 mL), the temperature was increased to 25° C. for 1 h. The resulting solution was concentrated under reduced pressure. The residue was applied on a silica gel column and eluted with 10:1 methylene chloride:MeOH to give tert-butyl (1R,5S)-6-[4-[4-[[5-amino-1-[2,3-dichloro-4-(fluoromethoxy)phenyl]imidazole-4-carbonyl]amino]-2-fluorobenzoyl]piperazine-1-carbonyl]-3-azabicyclo[3.1.0]hexane-3-carboxylate (80.0 mg, 0.11 mmol, 47.4% yield). MS [M-t-Bu+H]⁺: 678.1.

### Step 7: 5-amino-N-[4-[4-[(1R,5S)-3-azabicyclo[3.1.0]hexane-6-carbonyl]piperazine-1-carbonyl]-3-fluoro-phenyl]-1-[2,3-dichloro-4-(fluoromethoxy)phenyl]imidazole-4-carboxamide; 1:1 2,2,2-trifluoroacetic acid

A solution of tert-butyl (1R,5S)-6-[4-[4-[[5-amino-1-[2,3-dichloro-4-(fluoromethoxy)phenyl]imidazole-4-carbonyl]amino]-2-fluorobenzoyl]piperazine-1-carbonyl]-3-azabicyclo[3.1.0]hexane-3-carboxylate (70.0 mg, 0.1 mmol, 1.0 eq), trifluoroacetic acid (1.75 mL, 22.7 mmol, 238.37 eq) in methylene chloride (1 mL) was stirred at 25° C. for 1 h. The resulting solution was concentrated under reduced pressure. The crude product was purified by Prep-HPLC (Gemini C18 150×21.2 mm, 5 μm, MeCN— H₂O (0.1% TFA), 15-40) to afford 5-amino-N-[4-[4-[(1R,5S)-3-azabicyclo[3.1.0]hexane-6-carbonyl]piperazine-1-carbonyl]-3-fluoro-phenyl]-1-[2,3-dichloro-4-(fluoromethoxy)phenyl]imidazole-4-carboxamide; 1:1 2,2,2-trifluoroacetic acid (35.5 mg, 0.05 mmol, 49.7% yield). MS [M+H]⁺: 635.2.

### Compound 118

### 5-amino-N-[4-[4-[(1R,5S,6r)-3-azabicyclo[3.1.0]hexane-6-carbonyl]piperazine-1-carbonyl]-3-fluorophenyl]-1-[3-chloro-2-fluoro-4-(fluoromethoxy)phenyl]imidazole-4-carboxamide; 1:1 2,2,2-trifluoroacetic acid

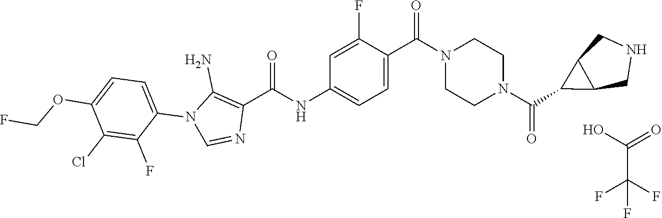

### Step 1: tert-butyl N-(3-chloro-2-fluoro-4-hydroxyphenyl) carbamate

To a solution of 4-amino-2-chloro-3-fluorophenol (2.0 g, 12.38 mmol, 1.0 eq) in THF (2 mL) was added aturated sodium bicarbonate (1 mL) at 25° C. under N₂ atmosphere, stirred for 2 h. Diluted with EtOAc (20 mL) and washed with H₂O (3×20 mL), dried over Na₂SO₄ and concentrated under reduced pressure and the residue was purified by flash column chromatography (85:15 petroleum ether:EtOAc) to give tert-butyl N-(3-chloro-2-fluoro-4-hydroxy-phenyl) carbamate (2.5 g, 9.6 mmol, 76% yield). MS [(M−C₄H₈)+H]⁺: 206.0.

### Step 2: tert-butyl N-[3-chloro-2-fluoro-4-(fluoromethoxy)phenyl]carbamate

To a solution of tert-butyl N-(3-chloro-2-fluoro-4-hydroxy-phenyl) carbamate (2.5 g, 9.55 mmol, 1.0 eq) were added N, N-diisopropylethylamine (3.7 g, 29 mmol, 3.0 eq) and fluoro(iodo)methane (2.29 g, 14.33 mmol, 1.5 eq) under N₂ atmosphere. The vessel was sealed and the mixture was stirred at 50° C. for 15 h. Concentrated under reduced pressure to give tert-butyl N-[3-chloro-2-fluoro-4-(fluoromethoxy)phenyl]carbamate (2.52 g, 8.58 mmol, 86% yield). The crude product was used directly without further purification. MS [(M−C₄H₈)+H]⁺: 238.0.

### Step 3: 3-chloro-2-fluoro-4-(fluoromethoxy)aniline

To a solution of tert-butyl N-[3-chloro-2-fluoro-4-(fluoromethoxy)phenyl]carbamate (2.52 g, 8.58 mmol, 1.0 eq) in methylene chloride (20 mL) was added trifluoroacetic acid (3.0 mL, 40.39 mmol, 4.71 eq) under N₂ atmosphere. The resulting mixture was stirred at 25° C. for 3 h. It was then concentrated under reduced pressure to afford 3-chloro-2-fluoro-4-(fluoromethoxy)aniline (1.3 g, 6.7 mmol, 78% yield). The crude product was used directly without further purification MS [M+H]⁺: 194.0.

### Step 4: ethyl 5-amino-1-[3-chloro-2-fluoro-4-(fluoromethoxy)phenyl]imidazole-4-carboxylate

To a solution of ethyl 2-amino-2-cyanoacetate; 4-methylbenzene-1-sulfonic acid (1551.45 mg, 5.17 mmol, 2.0 eq) in triethyl orthoformate (14.71 mL, 87.9 mmol, 34.03 eq) was added triethylamine (1045.46 mg, 10.33 mmol, 4.0 eq). The resulting mixture was stirred at 25° C. for 2 h. The solvent was removed under reduced pressure and the residue was diluted with MeCN (5 mL), then 3-chloro-2-fluoro-4-(fluoromethoxy)aniline (500.0 mg, 2.58 mmol, 1.0 eq) was added and the resulting mixture was stirred at 25° C. for 15 h. The solvent was removed under reduced pressure, diluted with EtOAc (50 mL), washed with H₂O (3×20 mL), dried over Na₂SO₄ and concentrated under reduced pressure. The residue was purified by flash column chromatography (30:70 petroleum ether:EtOAc) to give ethyl 5-amino-1-[3-chloro-2-fluoro-4-(fluoromethoxy)phenyl]imidazole-4-carboxylate (500.0 mg, 1.51 mmol, 55.4% yield). MS [M+H]⁺: 332.0.

### Step 5: 5-amino-1-[3-chloro-2-fluoro-4-(fluoromethoxy)phenyl]imidazole-4-carboxylic acid

To a solution of ethyl 5-amino-1-[3-chloro-2-fluoro-4-(fluoromethoxy)phenyl]imidazole-4-carboxylate (280.0 mg, 0.84 mmol, 1.0 eq) in 2-propanol (5 mL) were added potassium hydroxide (284.14 mg, 5.06 mmol, 6.0 eq) and water (2 mL) under N₂. The resulting mixture was stirred at 70° C. for 5 h. The solvent was removed under reduced pressure, then the aqueous layer was acidified with HCl (1 N) to pH 6-7. Isolation by filtration of the mixture gave 5-amino-1-[3-chloro-2-fluoro-4-(fluoromethoxy)phenyl]imidazole-4-carboxylic acid (200.0 mg, 0.66 mmol, 73.4% yield). The crude product was used directly without further purification. MS [M+H]⁺: 304.0

### Step 6: 5-amino-1-[3-chloro-2-fluoro-4-(fluoromethoxy)phenyl]imidazole-4-carbonyl chloride

A solution of 5-amino-1-[3-chloro-2-fluoro-4-(fluoromethoxy)phenyl]imidazole-4-carboxylic acid (50.0 mg, 0.16 mmol, 1.0 eq) in thionyl chloride (2.0 mL, 27.54 mmol, 167.23 eq) was stirred at 0° C. for 1 h. The resulting solution was concentrated under reduced pressure to give 5-amino-1-[3-chloro-2-fluoro-4-(fluoromethoxy)phenyl]imidazole-4-carbonyl chloride (51.0 mg, 0.16 mmol, 93.3% yield). MS [(M−Cl+OCH₃)+H]⁺: 318.0

### Step 7: tert-butyl (1R,5S)-6-[4-[4-[[5-amino-1-[3-chloro-2-fluoro-4-(fluoromethoxy)phenyl]imidazole-4-carbonyl]amino]-2-fluorobenzoyl]piperazine-1-carbonyl]-3-azabicyclo[3.1.0]hexane-3-carboxylate

A solution of tert-butyl (1R,5S)-6-[4-(4-amino-2-fluorobenzoyl)piperazine-1-carbonyl]-3-azabicyclo[3.1.0]hexane-3-carboxylate (201.41 mg, 0.47 mmol, 1.0 eq) and pyridine (0.6 mL, 7.46 mmol, 16.01 eq) in methylene chloride (2 mL) was stirred at 0° C., this was followed by the addition of 5-amino-1-[3-chloro-2-fluoro-4-(fluoromethoxy)phenyl]imidazole-4-carbonyl chloride (150.0 mg, 0.47 mmol, 1.0 eq) in methylene chloride (2 mL). The temperature was increased to 25° C. and stirred for 2 h. The solvent was removed under reduced pressure and the residue was purified by flash column chromatography (90:10 methylene chloride:MeOH) to give tert-butyl (1R,5S)-6-[4-[4-[[5-amino-1-[3-chloro-2-fluoro-4-(fluoromethoxy)phenyl]imidazole-4-carbonyl]amino]-2-fluorobenzoyl]piperazine-1-carbonyl]-3-azabicyclo[3.1.0]hexane-3-carboxylate (130.0 mg, 0.18 mmol, 38.1% yield). MS [(M−C₄H₈)+H]⁺: 662.2

### Step 8: 5-amino-N-[4-[4-[(1R,5S)-3-azabicyclo[3.1.0]hexane-6-carbonyl]piperazine-1-carbonyl]-3-fluoro-phenyl]-1-[3-chloro-2-fluoro-4-(fluoromethoxy)phenyl]imidazole-4-carboxamide; 1:1 2,2,2-trifluoroacetic acid

To a solution of tert-butyl (1R,5S)-6-[4-[4-[[5-amino-1-[3-chloro-2-fluoro-4-(fluoromethoxy)phenyl]imidazole-4-carbonyl]amino]-2-fluorobenzoyl]piperazine-1-carbonyl]-3-azabicyclo[3.1.0]hexane-3-carboxylate (110.0 mg, 0.15 mmol, 1.0 eq) in methylene chloride (4 mL) was added trifluoroacetic acid (1.0 mL, 13.46 mmol, 87.89 eq) under N₂ atmosphere. The resulting mixture was stirred at 25° C. for 2 h. The solvent was removed under reduced pressure and the residue was purified by prep-HPLC [Gemini-C18, 150×21.2 mm, 5 μm, MeCN-H₂O (0.1% TFA), 15%˜40%] to give 5-amino-N-[4-[4-[(1R,5S)-3-azabicyclo[3.1.0]hexane-6-carbonyl]piperazine-1-carbonyl]-3-fluoro-phenyl]-1-[3-chloro-2-fluoro-4-(fluoromethoxy)phenyl]imidazole-4-carboxamide;2,2,2-trifluoroacetic acid (81.73 mg, 0.11 mmol, 86.3% yield). MS [M+H]⁺: 618.0

### Compound 119

### 5-amino-N-[4-[4-[(1R,5S,6r)-3-azabicyclo[3.1.0]hexane-6-carbonyl]piperazine-1-carbonyl]-3-fluoro-phenyl]-1-[2-chloro-4-(fluoromethoxy)phenyl]imidazole-4-carboxamide; 1:1 2,2,2-trifluoroacetic acid

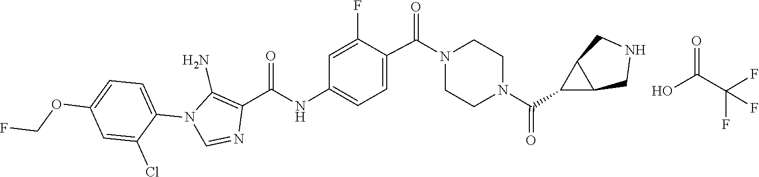

### Step 1: 2-chloro-4-(fluoromethoxy)-1-nitrobenzene

To a solution of 3-chloro-4-nitrophenol (1500.0 mg, 8.64 mmol, 1.0 eq) in MeCN (20 mL) were added N,N-diisopropylethylamine (3351.08 mg, 25.93 mmol, 3.0 eq) and fluoro (iodo)-methane (2073.42 mg, 12.96 mmol, 1.5 eq) under N₂ atmosphere. The vessel was sealed and the mixture was stirred at 50° C. for 15 h. After concentration under reduced pressure, the residue was purified by flash column chromatography on silica gel (90:10 petroleum ether:EtOAc) to give 2-chloro-4-(fluoromethoxy)-1-nitrobenzene (1200.0 mg, 5.84 mmol, 67.5% yield).

### Step 2: 2-chloro-4-(fluoromethoxy)aniline

To a solution of 2-chloro-4-(fluoromethoxy)-1-nitrobenzene (1780.0 mg, 8.66 mmol, 1.0 eq) in ethanol (25 mL) were added iron powder (2417.77 mg, 43.29 mmol, 5.0 eq), ammonium chloride (4631.62 mg, 86.59 mmol, 10.0 eq) and water (5 mL) under N₂ atmosphere. The resulting mixture was stirred at 50° C. for 10 h. The solid was filtered off and the filtrate was concentrated. The residue was diluted with EtOAc (50 mL) and washed with H₂O (3×25 mL), dried over Na₂SO₄ and concentrated under reduced pressure and the residue was purified by flash column chromatography (45:55 petroleum ether:EtOAc) to give 2-chloro-4-(fluoromethoxy)aniline (1300.0 mg, 7.4 mmol, 73.5% yield). MS [M+H]⁺: 176.1

### Step 3: ethyl 5-amino-1-[2-chloro-4-(fluoromethoxy)phenyl]imidazole-4-carboxylate

To a solution of ethyl 2-amino-2-cyanoacetate; 4-methylbenzene-1-sulfonic acid (4002.35 mg, 13.33 mmol, 2.0 eq) in triethyl orthoformate (35.0 mL, 209.2 mmol, 31.4 eq) was added triethylamine (2697.02 mg, 26.65 mmol, 4.0 eq). The resulting mixture was stirred at 83° C. for 2 h. The solvent was removed under reduced pressure and the residue was diluted with MeCN (30 mL), then 2-chloro-4-(fluoromethoxy)aniline (1170.0 mg, 6.66 mmol, 1.0 eq) was added and the resulting mixture was stirred at 25° C. for 15 h. The solvent was removed under reduced pressure, diluted with EtOAc (50 mL) and washed with H₂O (3×20 mL), dried over Na₂SO₄ and concentrated under reduced pressure and the residue was purified by flash column chromatography (55:45 petroleum ether:EtOAc) to give ethyl 5-amino-1-[2-chloro-4-(fluoromethoxy)phenyl]imidazole-4-carboxylate (1400.0 mg, 4.46 mmol, 63.6% yield). MS [M+H]⁺: 314.0

### Step 4: 5-amino-1-[2-chloro-4-(fluoromethoxy)phenyl]imidazole-4-carboxylic acid

To a solution of ethyl 5-amino-1-[2-chloro-4-(fluoromethoxy)phenyl]imidazole-4-carboxylate (200.0 mg, 0.64 mmol, 1.0 eq) in methanol (6 mL) were added lithium hydroxide hydrate (267.51 mg, 6.38 mmol, 10.0 eq) and water (1.3 mL, 72 mmol, 114 eq) under N₂. The resulting mixture was stirred at 50° C. for 15 h. The solvent was removed under reduced pressure, then diluted with H₂O (20 mL), extracted with EtOAc (3×10 mL) and the organic layers were discarded. The aqueous layer was acidified with HCl (4 N), then extracted with EtOAc, dried over Na₂SO₄ and concentrated to give 5-amino-1-[2-chloro-4-(fluoromethoxy)phenyl]imidazole-4-carboxylic acid (182.0 mg, 0.64 mmol, 94% yield). The crude product was used directly without further purification. MS [M+H]⁺: 286.0

### Step 5: 5-amino-1-[2-chloro-4-(fluoromethoxy)phenyl]imidazole-4-carbonyl chloride

5-amino-1-[2-chloro-4-(fluoromethoxy)phenyl]imidazole-4-carboxylic acid (182.0 mg, 0.64 mmol, 1.0 eq) was dissolved in thionyl chloride (3.0 mL, 41.3 mmol, 64.83 eq) at 0° C. under N₂ atmosphere. The resulting mixture was stirred at 25° C. for 3 h. The solvent was removed under reduced pressure to give 5-amino-1-[2-chloro-4-(fluoromethoxy)phenyl]imidazole-4-carbonyl chloride (190.0 mg, 0.62 mmol, 71.6% yield). MS [M−Cl+OCH₃+H]⁺: 300.0

### Step 6: tert-butyl (1R,5S)-6-[4-[4-[[5-amino-1-[2-chloro-4-(fluoromethoxy)phenyl]imidazole-4-carbonyl]amino]-2-fluorobenzoyl]piperazine-1-carbonyl]-3-azabicyclo[3.1.0]hexane-3-carboxylate

To a solution of tert-butyl (1R,5S)-6-[4-(4-amino-2-fluorobenzoyl)piperazine-1-carbonyl]-3-azabicyclo[3.1.0]hexane-3-carboxylate (114.84 mg, 0.27 mmol, 0.85 eq) in methylene chloride (2 mL) were added pyridine (0.51 mL, 6.25 mmol, 20.0 eq) and 5-amino-1-[2-chloro-4-(fluoromethoxy)phenyl]imidazole-4-carbonyl chloride (95.0 mg, 0.31 mmol, 1.0 eq) at 0° C. under N₂ atmosphere, then the resulting mixture was allowed to warm to 25° C. and stirred for 0.5 h. The solvent was removed under reduced pressure, diluted with EtOAc (25 mL) and washed with H₂O (3×10 mL), dried over Na₂SO₄ and concentrated under reduced pressure. The residue was purified by flash column chromatography (97:3 methylene chloride:MeOH) to give tert-butyl (1R,5S)-6-[4-[4-[[5-amino-1-[2-chloro-4-(fluoromethoxy)phenyl]imidazole-4-carbonyl]amino]-2-fluorobenzoyl]piperazine-1-carbonyl]-3-azabicyclo[3.1.0]hexane-3-carboxylate (120.0 mg, 0.17 mmol, 54.9% yield). MS [M+H]⁺: 700.1

### Step 7: 5-amino-N-[4-[4-[(1R,5S)-3-azabicyclo[3.1.0]hexane-6-carbonyl]piperazine-1-carbonyl]-3-fluorophenyl]-1-[2-chloro-4-(fluoromethoxy)phenyl]imidazole-4-carboxamide; 1:1 2,2,2-trifluoroacetic acid

To a solution of tert-butyl (1R,5S)-6-[4-[4-[[5-amino-1-[2-chloro-4-(fluoromethoxy)phenyl]imidazole-4-carbonyl]amino]-2-fluorobenzoyl]piperazine-1-carbonyl]-3-azabicyclo[3.1.0]hexane-3-carboxylate (120.0 mg, 0.17 mmol, 1.0 eq) in methanol (1 mL) was added trifluoroacetic acid (2.0 mL, 8.0 mmol, 47 eq). The resulting mixture was stirred at 25° C. for 0.5 h. The solvent was removed under reduced pressure, the residue was purified by prep-HPLC [Gemini-C18, 150×21.2 mm, 5 μm, MeCN-H₂O (0.1% TFA), 20%˜45%] to give 5-amino-N-[4-[4-[(1R,5S)-3-azabicyclo[3.1.0]hexane-6-carbonyl]piperazine-1-carbonyl]-3-fluoro-phenyl]-1-[2-chloro-4-(fluoromethoxy)phenyl]imidazole-4-carboxamide;2,2,2-trifluoroacetic acid (82.3 mg, 0.12 mmol, 66.6% yield). MS [M+H]⁺: 600.1

### Compound 120

### N-(4-(4-(1R,5S,6r)-3-azabicyclo[3.1.0]hexane-6-carbonyl)piperazine-1-carbonyl)-3-fluorophenyl)-5-amino-1-(3-chloro-4-(fluoromethoxy)phenyl)-1H-imidazole-4-carboxamide; 1:1 trifluoroacetate

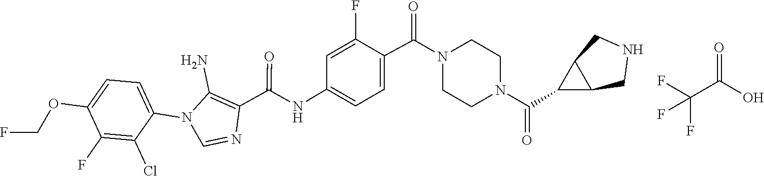

### Step 1: 2-chloro-1-(fluoromethoxy)-4-nitrobenzene

To a solution of 2-chloro-4-nitrophenol (1500.0 mg, 8.64 mmol, 1.0 eq) in MeCN (20 mL) was added N, N-diisopropylethylamine (3351.34 mg, 25.93 mmol, 3.0 eq) and fluoro(iodo)methane (2764.55 mg, 17.29 mmol, 2.0 eq). The reaction was stirred at 50° C. for 5 h and then concentrated to dryness. The residue was taken up in EtOAc (30 mL) and the organics washed with 2×30 ml water followed by 30 mL brine. The organics were then separated and dried overMgSO₄ before concentrating to dryness. The crude was then purified by flash column chromatography eluting with 50% EtOAc in petroleum ether to afford 2-chloro-1-(fluoromethoxy)-4-nitrobenzene (1500.0 mg, 7.3 mmol, 84.4% yield).

### Step 2: 3-chloro-4-(fluoromethoxy)aniline

To a solution of 2-chloro-1-(fluoromethoxy)-4-nitrobenzene (1500.0 mg, 7.3 mmol, 1.0 eq) in ethanol (20 mL) and water (2 mL) added iron powder (2043.1 mg, 36.48 mmol, 5.0 eq) and ammonium chloride (1933.65 mg, 36.48 mmol, 5.0 eq). The reaction was stirred at 70° C. for 3 h. The solid was filtered off and the filtrate was concentrated under reduced pressure to afford 3-chloro-4-(fluoromethoxy)aniline (960 mg, 66.31% yield, 88.5% purity) which was used in next step without further purification. MS [M+H]⁺: 176.1.

### Step 3: ethyl 5-amino-1-(3-chloro-4-(fluoromethoxy)phenyl)-1H-imidazole-4-carboxylate

To a solution of ethyl 2-amino-2-cyanoacetate; 4-methylbenzene-1-sulfonic acid (4618.09 mg, 15.38 mmol, 3.0 eq) in triethyl orthoformate (37.22 mL, 222.44 mmol, 43.4 eq) was added triethylamine (3111.94 mg, 30.75 mmol, 6.0 eq). The resulting mixture was stirred at 80° C. for 2 h. The solvent was then removed under reduced pressure, and the residue was diluted with MeCN (40 mL). 3-Chloro-4-(fluoromethoxy)aniline (900.0 mg, 5.13 mmol, 1.0 eq) was added and the resulting mixture was stirred at 25° C. for 15 h. The reaction was then concentrated to dryness. The residue was taken up in EtOAc (30 mL), and the organics washed with 2×30 mL water followed by 30 mL brine. The organics were then separated and dried (MgSO₄) before concentrating to dryness. The crude was then purified by flash column chromatography eluting with 3% MeOH in methylene chloride to afford ethyl 5-amino-1-(3-chloro-4-(fluoromethoxy)phenyl)-1H-imidazole-4-carboxylate (1200 mg, 72.23%, 96.8% purity). MS [M+H]⁺: 314.0

### Step 4: 5-amino-1-(3-chloro-4-(fluoromethoxy)phenyl)-1H-imidazole-4-carboxylic acid

To a solution of ethyl 5-amino-1-[3-chloro-4-(fluoromethoxy)phenyl]imidazole-4-carboxylate (1200.0 mg, 3.83 mmol, 1.0 eq) in methanol (15 mL) and water (1.5 mL) was added lithium hydroxide (0.36 mL, 38 mmol, 10.0 eq). The reaction was stirred at 60° C. for 6 h at which time the solvent was evaporated. The residue was diluted with water (10 mL), and the solution was extracted with ethyl acetate (2×10 mL). The aqueous layer was acidified to pH 5 with HCl (2 M), and the product was collected by filtration to afford 5-amino-1-[3-chloro-4-(fluoromethoxy)phenyl]imidazole-4-carboxylic acid (870.0 mg, 3.05 mmol, 69.9% yield). MS [M+H]⁺: 286.0

### Step 5: 5-amino-1-(3-chloro-4-(fluoromethoxy)phenyl)-1H-imidazole-4-carbonyl chloride

To 5-amino-1-(3-chloro-4-(fluoromethoxy)phenyl)-1H-imidazole-4-carboxylic acid (200.0 mg, 0.7 mmol, 1.0 eq) under N₂ was added thionyl chloride (5 mL) at 20° C. The reaction mixture was stirred at 20° C. for 1 hour under N₂. The resulting solution was evaporated to dryness to afford the title compound 5-amino-1-(3-chloro-4-(fluoromethoxy)phenyl)-1H-imidazole-4-carbonyl chloride (200 mg, 50.6% yield, 53.9% purity) which was used without further purification. MS [M−Cl+OCH₃+H]⁺: 300.0

### Step 6: tert-butyl (1R,5S,6r)-6-(4-(4-(5-amino-1-(3-chloro-4-(fluoromethoxy)phenyl)-1H-imidazole-4-carboxamido)-2-fluorobenzoyl)piperazine-1-carbonyl)-3-azabicyclo[3.1.0]hexane-3-carboxylate

To a solution of tert-butyl (1R,5S)-6-[4-(4-amino-2-fluorobenzoyl)piperazine-1-carbonyl]-3-azabicyclo[3.1.0]hexane-3-carboxylate (142.22 mg, 0.33 mmol, 1.0 eq) in methylene chloride (3 mL) was added pyridine (0.27 mL, 3.29 mmol, 10.0 eq) followed by 5-amino-1-[3-chloro-4-(fluoromethoxy)phenyl]imidazole-4-carbonyl chloride (100.0 mg, 0.33 mmol, 1.0 eq) at 0° C. The reaction mixture was stirred at 25° C. for 3 h at which time it was concentrated to dryness. The residue was taken up in methylene chloride (20 mL), and the organics washed with 2×20 mL water and 20 mL brine. The organics were then separated and dried (MgSO₄) before concentrating to dryness. The crude was purified by flash column chromatography eluting with EtOAc to afford tert-butyl (1R,5S,6r)-6-(4-(4-(5-amino-1-(3-chloro-4-(fluoromethoxy)phenyl)-1H-imidazole-4-carboxamido)-2-fluorobenzoyl)piperazine-1-carbonyl)-3-azabicyclo[3.1.0]hexane-3-carboxylate (10 mg, 43% yield, 100% purity). MS [M+H]⁺: 700.0

### Step 7: N-(4-(4-((1R,5S,6r)-3-azabicyclo[3.1.0]hexane-6-carbonyl)piperazine-1-carbonyl)-3-fluorophenyl)-5-amino-1-(3-chloro-4-(fluoromethoxy)phenyl)-1H-imidazole-4-carboxamide 1:1 2,2,2-trifluoroacetate

To a solution of tert-butyl (1R,5S)-6-[4-[4-[[5-amino-1-[3-chloro-4-(fluoromethoxy)phenyl]imidazole-4-carbonyl]amino]-2-fluorobenzoyl]piperazine-1-carbonyl]-3-azabicyclo[3.1.0]hexane-3-carboxylate (90.0 mg, 0.13 mmol, 1.0 eq) in methylene chloride (3 mL) was added trifluoroacetic acid (1.0 mL). The reaction stirred at 25° C. for 3 h. The reaction was concentrated to dryness to afford crude product. The crude was then purified by HPLC (Gemini-C18 150×21.2 mm, 5 μm, MeCN-H₂O (0.1% TFA), 20-60) to afford 5-amino-1-[3-chloro-4-(fluoromethoxy)phenyl]-N-[3-fluoro-4-[4-[(1R,5S)-3-azabicyclo[3.1.0]hexane-6-carbonyl]piperazine-1-carbonyl]phenyl]imidazole-4-carboxamide; 1:1 2,2,2-trifluoroacetic acid (38.3 mg, 0.05 mmol, 41.7% yield). MS [M+H]⁺: 600.0

### Compound 121

### 5-amino-1-[4-(difluoromethoxy)-2,3-difluoro-phenyl]-N-[3-fluoro-4-[4-[(1R,5S,6r)-3-azabicyclo[3.1.0]hexane-6-carbonyl]piperazine-1-carbonyl]phenyl]imidazole-4-carboxamide; 1:1 2,2,2-trifluoroacetic acid

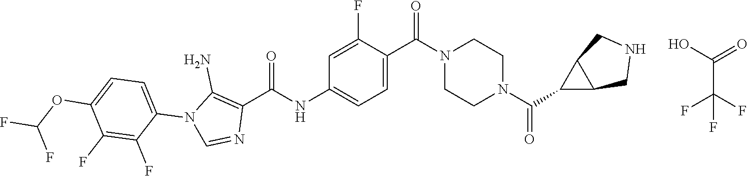 [SMILES: Nc1c(C(=O)Nc2ccc(C(=O)N3CCN(C(=O)[C@H]4[C@@H]5CNC[C@@H]54)CC3)c(F)c2)ncn1-c1ccc(OC(F)F)c(F)c1F ; O=C(O)C(F)(F)F]

### Step 1: ethyl 5-amino-1-[4-(difluoromethoxy)-2,3-difluorophenyl]imidazole-4-carboxylate

To a solution of ethyl 2-amino-2-cyanoacetate; 4-methylbenzene-1-sulfonic acid (4.62 g, 15.38 mmol, 3.0 eq) in triethyl orthoformate (42.15 mL, 251.95 mmol, 49.16 eq) was added triethylamine (3111.78 mg, 30.75 mmol, 6.0 eq). The resulting mixture was stirred at 83° C. for 2 h. The solvent was removed under reduced pressure, and the residue was diluted with MeCN (50 mL). Next, 4-(difluoromethoxy)-2,3-difluoro-aniline (1.0 g, 5.13 mmol, 1.0 eq) was added, and the resulting mixture was stirred at 25° C. for 15 h. After concentration under reduced pressure, the residue was purified by flash column chromatography on silica gel (55:45 petroleum ether:EtOAc elution) to give ethyl 5-amino-1-[4-(difluoromethoxy)-2,3-difluorophenyl]imidazole-4-carboxylate (1.2 g, 3.60 mmol, 67.5% yield). MS [M+H]⁺: 334.0

### Step 2: 5-amino-1-[4-(difluoromethoxy)-2,3-difluoro-phenyl]imidazole-4-carboxylic acid

To a solution of sodium hydroxide (720.15 mg, 18.01 mmol, 5.0 eq) was added ethyl 5-amino-1-[4-(difluoromethoxy)-2,3-difluorophenyl]imidazole-4-carboxylate (1200.0 mg, 3.6 mmol, 1.0 eq) in isopropyl alcohol (8 mL) and water (4 mL). The resulting mixture was stirred at 70° C. for 4 h. The mixture was adjusted to pH 5-6 with 2N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure to give 5-amino-1-[4-(difluoromethoxy)-2,3-difluorophenyl]imidazole-4-carboxylic acid (750.0 mg, 2.46 mmol, 36.4% yield). MS [M+H]⁺: 306.1.

### Step 3: 5-amino-1-[4-(difluoromethoxy)-2,3-difluorophenyl]imidazole-4-carbonyl chloride

A solution of 5-amino-1-[4-(difluoromethoxy)-2,3-difluorophenyl]imidazole-4-carboxylic acid (100.0 mg, 0.33 mmol, 1.0 eq) in thionyl chloride (389.82 mg, 3.28 mmol, 10.0 eq) was stirred at 25° C. for 1 h. The resulting solution was concentrated under reduced pressure to give 5-amino-1-[4-(difluoromethoxy)-2,3-difluorophenyl]imidazole-4-carbonyl chloride (100.0 mg, 0.31 mmol, 69.7% yield). MS [(M−Cl+OCH₃)+H]⁺: 320.0

### Step 4: tert-butyl (1R,5S)-6-[4-[4-[[5-amino-1-[4-(difluoromethoxy)-2,3-difluorophenyl]imidazole-4-carbonyl]amino]-2-fluorobenzoyl]piperazine-1-carbonyl]-3-azabicyclo[3.1.0]hexane-3-carboxylate

To a solution of tert-butyl (1R,5S)-6-[4-(4-amino-2-fluorobenzoyl)piperazine-1-carbonyl]-3-azabicyclo[3.1.0]hexane-3-carboxylate (133.64 mg, 0.31 mmol, 1.0 eq) and pyridine (0.25 mL, 3.09 mmol, 10.0 eq) in methylene chloride (2 mL) was added 5-amino-1-[4-(difluoromethoxy)-2,3-difluorophenyl]imidazole-4-carbonyl chloride (100.0 mg, 0.31 mmol, 1.0 eq). The resulting mixture was stirred at 25° C. for 3 h. The reaction was concentrated to dryness. The residue was taken up in EtOAc (5 mL) and the organics washed with 2×3 ml water followed by 3 ml brine. The organics were then separated and dried (Na₂SO₄) before concentrating to dryness. The crude was purified by flash column chromatography eluting with 90% EtOAc in petroleum ether elution to give tert-butyl (1R,5S)-6-[4-[4-[[5-amino-1-[4-(difluoromethoxy)-2,3-difluorophenyl]imidazole-4-carbonyl]amino]-2-fluorobenzoyl]piperazine-1-carbonyl]-3-azabicyclo[3.1.0]hexane-3-carboxylate (140.0 mg, 0.19 mmol, 47.6% yield). MS [(M−C₄H₈)+H]⁺: 664.2.

### Step 5: 5-amino-1-[4-(difluoromethoxy)-2,3-difluorophenyl]-N-[3-fluoro-4-[4-[(1R,5S)-3-azabicyclo[3.1.0]hexane-6-carbonyl]piperazine-1-carbonyl]phenyl]imidazole-4-carboxamide; 1:1 2,2,2-trifluoroacetic acid

A solution of tert-butyl (1R,5S)-6-[4-[4-[[5-amino-1-[4-(difluoromethoxy)-2,3-difluorophenyl]imidazole-4-carbonyl]amino]-2-fluorobenzoyl]piperazine-1-carbonyl]-3-azabicyclo[3.1.0]hexane-3-carboxylate (50.0 mg, 0.07 mmol, 1.0 eq) and trifluoroacetic acid (1.0 mL, 12.98 mmol, 186.82 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN— H₂O (0.1% TFA), 20-50) to give 5-amino-1-[4-(difluoromethoxy)-2,3-difluorophenyl]-N-[3-fluoro-4-[4-[(1R,5S)-3-azabicyclo[3.1.0]hexane-6-carbonyl]piperazine-1-carbonyl]phenyl]imidazole-4-carboxamide; 1:1 2,2,2-trifluoroacetic acid (13.8 mg, 0.02 mmol, 26% yield). MS [M+H]⁺: 620.3.

### Compound 122

### 5-amino-N-[3-chloro-4-[4-(piperazine-1-carbonyl)piperazine-1-carbonyl]phenyl]-1-[2-(difluoromethyl)-3-fluoro-4-(fluoromethoxy)phenyl]imidazole-4-carboxamide; 1:1 2,2,2-trifluoroacetic acid

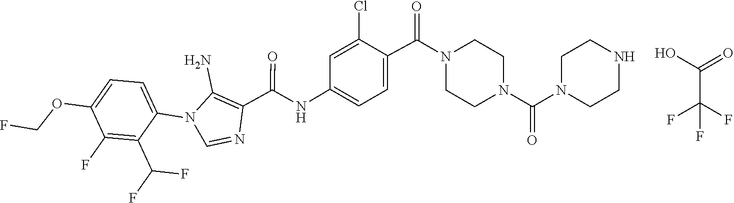 [SMILES: Nc1c(C(=O)Nc2ccc(C(=O)N3CCN(C(=O)N4CCNCC4)CC3)c(Cl)c2)ncn1-c1ccc(OCF)c(F)c1C(F)F ; O=C(O)C(F)(F)F]

### Step 1: 5-amino-1-[2-(difluoromethyl)-3-fluoro-4-(fluoromethoxy)phenyl]imidazole-4-carbonyl chloride

A solution of 5-amino-1-[2-(difluoromethyl)-3-fluoro-4-(fluoromethoxy)phenyl]imidazole-4-carboxylic acid (20.0 mg, 0.06 mmol, 1.0 eq) in thionyl chloride (74.54 mg, 0.63 mmol, 10.0 eq) was stirred at 25° C. for 1 h. The resulting solution was concentrated under reduced pressure to give 5-amino-1-[2-(difluoromethyl)-3-fluoro-4-(fluoromethoxy)phenyl]imidazole-4-carbonyl chloride (20.0 mg, 0.06 mmol, 59.8% yield). MS [(M−Cl+OCH₃)+H]⁺: 334.2.

### Step 2: tert-butyl 4-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]piperazine-1-carboxylate

To a solution of tert-butyl 4-[4-(4-amino-2-chlorobenzoyl)piperazine-1-carbonyl]piperazine-1-carboxylate (46.85 mg, 0.1 mmol, 1.0 eq) and pyridine (0.08 mL, 1.04 mmol, 10.0 eq) in methylene chloride (2 mL) was added 5-amino-1-[2-(difluoromethyl)-3-fluoro-4-(fluoromethoxy)phenyl]imidazole-4-carbonyl chloride (35.0 mg, 0.1 mmol, 1.0 eq). The resulting mixture was stirred at 25° C. for 3 h. The reaction was concentrated to dryness. The residue was taken up in EtOAc (5 ml) and the organics washed with 2×3 ml water followed by 3 ml brine. The organics were then separated and dried (Na₂SO₄) before concentrating to dryness. The crude was purified by flash column chromatography eluting with 90% EtOAc in petroleum ether to give tert-butyl 4-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]piperazine-1-carboxylate (60.0 mg, 0.08 mmol, 76.9% purity). MS [M+H]⁺: 753.0

### Step 3: 5-amino-N-[3-chloro-4-[4-(piperazine-1-carbonyl)piperazine-1-carbonyl]phenyl]-1-[2-(difluoromethyl)-3-fluoro-4-(fluoromethoxy)phenyl]imidazole-4-carboxamide; 1:1 2,2,2-trifluoroacetic acid

A solution of tert-butyl 4-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]piperazine-1-carboxylate (40.0 mg, 0.05 mmol, 1.0 eq) and trifluoroacetic acid (1.0 mL, 12.98 mmol, 244.39 eq) in methylene chloride (3 mL) was stirred at 25° C. for 1 h. The resulting solution was concentrated under reduced pressure. The residue was purified by Prep-HPLC (AQ-C18, MeCN— H₂O (0.1% TFA)) to give 5-amino-N-[3-chloro-4-[4-(piperazine-1-carbonyl)piperazine-1-carbonyl]phenyl]-1-[2-(difluoromethyl)-3-fluoro-4-(fluoromethoxy)phenyl]imidazole-4-carboxamide; 1:1 2,2,2-trifluoroacetic acid (6.1 mg, 0.01 mmol, 14.9% yield). MS [M+H]⁺: 652.9

### Compound 123

### 5-amino-N-(3-chloro-4-(4-(piperazine-1-carbonyl)piperazine-1-carbonyl)phenyl)-1-(3-(difluoromethyl)-2-fluoro-4-(fluoromethoxy)phenyl)-1H-imidazole-4-carboxamide; 1:1 2,2,2-trifluoroacetate acid

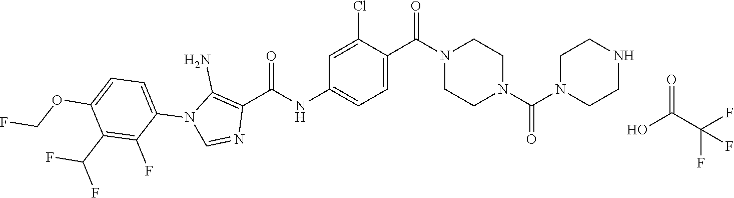 [SMILES: Nc1c(C(=O)Nc2ccc(C(=O)N3CCN(C(=O)N4CCNCC4)CC3)c(Cl)c2)ncn1-c1ccc(OCF)c(C(F)F)c1F ; O=C(O)C(F)(F)F]

### Step 1: 3-fluoro-1-methoxy-2-methyl-4-nitrobenzene

A solution of 1,3-difluoro-2-methyl-4-nitrobenzene (3.0 g, 17.33 mmol, 1.0 eq) and sodium carbonate (1.84 g, 17.33 mmol, 1.0 eq) in methanol (30 mL) was stirred at 60° C. for 48 h. The resulting solution was concentrated under reduced pressure. The residue was applied on a silica gel column and eluted with 30:1 petroleum ether:EtOAc to provide 3-fluoro-1-methoxy-2-methyl-4-nitrobenzene (1.5 g, 8.1 mmol, 46.8% yield). MS [M+H]⁺: 186.0

### Step 2: 3-fluoro-2-methyl-4-nitrophenol

A solution of 3-fluoro-1-methoxy-2-methyl-4-nitrobenzene (1.4 g, 7.6 mmol, 1.0 eq) and boron tribromide (10 mL) in methylene chloride (10 mL) was stirred at 80° C. for 16 h. After cooling to room temperature, the reaction mixture was quenched with water (20 mL) and extracted with methylene chloride (20 mL). The combined extracts were washed with water (20 mL) and brine (40 mL), dried (Na₂SO₄), and concentrated to give 2,3-dichloro-4-nitrophenol (1.1 g, 6.4 mmol, 82% yield). MS [M−H]⁺: 170.0

### Step 3: 3-fluoro-1-(fluoromethoxy)-2-methyl-4-nitrobenzene

To a solution of 3-fluoro-2-methyl-4-nitrophenol (1.1 g, 6.43 mmol, 1.0 eq) in MeCN (20 mL) were added N, N-diisopropylethylamine (3.36 mL, 19.28 mmol, 3.0 eq) and fluoro(iodo)methane (1.54 g, 9.64 mmol, 1.5 eq) under N₂ atmosphere. The vessel was sealed and the mixture was stirred at 50° C. for 15 h. After concentration under reduced pressure, the residue was purified by flash column chromatography on silica gel (95:5 petroleum ether:EtOAc) to give 3-fluoro-1-(fluoromethoxy)-2-methyl-4-nitrobenzene (1.1 g, 5.4 mmol, 83% yield). MS [M+H]⁺: 204.0

### Step 4: 2-(bromomethyl)-3-fluoro-1-(fluoromethoxy)-4-nitrobenzene

A solution of 3-fluoro-1-(fluoromethoxy)-2-methyl-4-nitrobenzene (1.0 g, 4.92 mmol, 1.0 eq), N-bromosuccinimide (1.31 g, 7.38 mmol, 1.5 eq), dibenzoyl peroxide (119.13 mg, 0.49 mmol, 0.1 eq) in tetrachloromethane (15.0 mL) was stirred at 80° C. for 15 h under N₂ atmosphere. The resulting solution was concentrated under reduced pressure. The residue was applied on a silica gel column and eluted with 1:2 EtOAc:petroleum ether elution to give 2-(bromomethyl)-3-fluoro-1-(fluoromethoxy)-4-nitrobenzene (1.23 g, 4.36 mmol, 84.12% yield).

### Step 5: 2-fluoro-6-(fluoromethoxy)-3-nitro-benzaldehyde

To a solution of 2-(bromomethyl)-3-fluoro-1-(fluoromethoxy)-4-nitrobenzene (1.2 g, 4.2 mmol, 1.0 eq) in MeCN (20 mL) were added N-methylmorpholine N-oxide (1.2 g, 10.2 mmol, 2.4 eq) and 4 Å molecular sieves (2.4 g) was stirred at 25° C. for 15 h under N₂ atmosphere. The resulting solution was concentrated under reduced pressure. The residue was applied on a silica gel column and eluted with 1:2 EtOAc:petroleum ether to give 2-fluoro-6-(fluoromethoxy)-3-nitrobenzaldehyde (880.0 mg, 4.05 mmol, 95.2% yield). MS [M+H]⁺: 218.1

### Step 6: 2-(difluoromethyl)-3-fluoro-1-(fluoromethoxy)-4-nitrobenzene

To a solution of 2-fluoro-6-(fluoromethoxy)-3-nitrobenzaldehyde (880.0 mg, 4.05 mmol, 1.0 eq) in bis(2-methoxyethyl) aminosulfur trifluoride (10.0 mL) were added methanol (20.29 mg, 0.41 mmol, 0.1 eq), then the solution stirred at 50° C. for 2 h. The solution was quenched with 20 mL MeOH. The resulting mixture was concentrated under reduced pressure. The residue was applied on a silica gel column and eluted with 2:1 EtOAc:petroleum ether to give 2-(difluoromethyl)-3-fluoro-1-(fluoromethoxy)-4-nitrobenzene (800.0 mg, 3.35 mmol, 82.6% yield).

### Step 7: 2-fluoro-4-(fluoromethoxy)-3-methyl-aniline

A solution of 2-(difluoromethyl)-3-fluoro-1-(fluoromethoxy)-4-nitrobenzene (780.0 mg, 3.26 mmol, 1.0 eq), ammonium chloride (872.41 mg, 16.31 mmol, 5.0 eq) and iron dust (913.35 mg, 16.31 mmol, 5.0 eq) in ethanol (10 mL) and water (10 mL) was heated at 50° C. for 2 h. The solid was filtered off, and the filtrate was concentrated under reduced pressure. The residue was diluted with water, then extracted with 2×20 mL of ethyl acetate. The organic layers were combined, washed with brine, dried and concentrated under reduced pressure to afford 3-(difluoromethyl)-2-fluoro-4-(fluoromethoxy)aniline (410.0 mg, 1.96 mmol, 54.1% yield). MS [M+H]⁺: 210.0

### Step 8: ethyl 5-amino-1-[3-(difluoromethyl)-2-fluoro-4-(fluoromethoxy)phenyl]imidazole-4-carboxylate

To a solution of ethyl 2-amino-2-cyanoacetate; 4-methylbenzene-1-sulfonic acid (861.61 mg, 2.87 mmol, 2.0 eq) in MeCN (5 mL) were added triethylamine (0.8 mL, 5.72 mmol, 4.0 eq) and triethyl orthoformate (2125.8 mg, 14.3 mmol, 10.0 eq). The resulting mixture was stirred at 80° C. for 2 h. The solvent was removed under reduced pressure and the residue was diluted with MeCN (5 mL). Next, 3-(difluoromethyl)-2-fluoro-4-(fluoromethoxy)aniline (300.0 mg, 1.43 mmol, 1.0 eq) was added and the resulting mixture was stirred at 40° C. for 15 h. After concentration under reduced pressure, the residue was purified by flash column chromatography on silica gel (1:3 petroleum ether:EtOAc) to give ethyl 5-amino-1-[3-(difluoromethyl)-2-fluoro-4-(fluoromethoxy)phenyl]imidazole-4-carboxylate (300.0 mg, 0.86 mmol, 30.1% yield). MS [M+H]⁺: 348.0

### Step 9: 5-amino-1-[3-(difluoromethyl)-2-fluoro-4-(fluoromethoxy)phenyl]imidazole-4-carboxylic acid

A solution of ethyl 5-amino-1-[3-(difluoromethyl)-2-fluoro-4-(fluoromethoxy)phenyl]imidazole-4-carboxylate (190.0 mg, 0.55 mmol, 1.0 eq), sodium hydroxide (109.42 mg, 2.74 mmol, 5.0 eq) in the isopropyl alcohol (3 mL) and water (1 mL) was stirred at 50° C. for 12 h. The residue was diluted with 10 ml of water, then adjusted to pH 5˜6 with HCl (2 M) and extracted with 3×10 mL of EtOAc. The organic layers were combined and concentrated under reduced pressure to give 5-amino-1-[3-(difluoromethyl)-2-fluoro-4-(fluoromethoxy)phenyl]imidazole-4-carboxylic acid (150.0 mg, 0.47 mmol, 77.3% yield). MS [M+H]⁺: 320.1

### Step 10: 5-amino-1-[3-(difluoromethyl)-2-fluoro-4-(fluoromethoxy)phenyl]imidazol-4-carbonyl chloride

A solution of 5-amino-1-[3-(difluoromethyl)-2-fluoro-4-(fluoromethoxy)phenyl]imidazole-4-carboxylic acid (40.0 mg, 0.13 mmol, 1.0 eq) in thionyl chloride (2.0 mL) was stirred at 25° C. for 1 h. The resulting solution was concentrated under reduced pressure to give 5-amino-1-[3-(difluoromethyl)-2-fluoro-4-(fluoromethoxy)phenyl]imidazole-4-carbonyl chloride (40.0 mg, 0.12 mmol, 85.1% yield). MS [M−Cl+OMe+H]⁺: 334.0

### Step 11: tert-butyl 4-[4-[4-[[5-amino-1-[3-(difluoromethyl)-2-fluoro-4-(fluoromethoxy)phenyl]imidazole-4-carbonyl]amino]-2-chlorobenzoyl]piperazine-1-carbonyl]piperazine-1-carboxylate

A solution of tert-butyl 4-[4-(4-amino-2-chlorobenzoyl)piperazine-1-carbonyl]piperazine-1-carboxylate (53.54 mg, 0.12 mmol, 1.0 eq) and pyridine (0.1 mL, 1.18 mmol, 10.0 eq) in methylene chloride (2 mL) was stirred at 25° C. Next, 5-amino-1-[3-(difluoromethyl)-2-fluoro-4-(fluoromethoxy)phenyl]imidazole-4-carbonyl chloride (40.0 mg, 0.12 mmol, 1.0 eq) in methylene chloride (2 mL) was added, and the temperature was increased to 25° C. for 1 h. The resulting solution was concentrated under reduced pressure. The residue was applied on a silica gel column and eluted with 10:1 methylene chloride:MeOH to give tert-butyl 4-[4-[4-[[5-amino-1-[3-(difluoromethyl)-2-fluoro-4-(fluoromethoxy)phenyl]imidazole-4-carbonyl]amino]-2-chlorobenzoyl]piperazine-1-carbonyl]piperazine-1-carboxylate (60.0 mg, 0.08 mmol, 6.7% yield). MS [M+H]⁺: 753.1

### Step 12: 5-amino-N-[3-chloro-4-[4-(piperazin-4-ium-1-carbonyl)piperazine-1-carbonyl]phenyl]-1-[3-(difluoromethyl)-2-fluoro-4-(fluoromethoxy)phenyl]imidazole-4-carboxamide; 1:1 2,2,2-trifluoroacetate acid

A solution of tert-butyl 4-[4-[4-[[5-amino-1-[3-(difluoromethyl)-2-fluoro-4-(fluoromethoxy)phenyl]imidazole-4-carbonyl]amino]-2-chlorobenzoyl]piperazine-1-carbonyl]piperazine-1-carboxylate (50.0 mg, 0.07 mmol, 1.0 eq), trifluoroacetic acid (1.0 mL) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40) to afford 5-amino-N-[3-chloro-4-[4-(piperazin-4-ium-1-carbonyl)piperazine-1-carbonyl]phenyl]-1-[3-(difluoromethyl)-2-fluoro-4-(fluoromethoxy)phenyl]imidazole-4-carboxamide;1:1 2,2,2-trifluoroacetate acid (7.5 mg, 0.01 mmol, 14.6% yield). MS [M+H]⁺: 653.2

### Compound 124

### 5-amino-N-[3-chloro-4-[4-[(1R,5S)-3-azabicyclo[3.1.0]hexane-6-carbonyl]piperazine-1-carbonyl]phenyl]-1-[2-(difluoromethyl)-3-fluoro-4-(fluoromethoxy)phenyl]imidazole-4-carboxamide; 1:1 formic acid

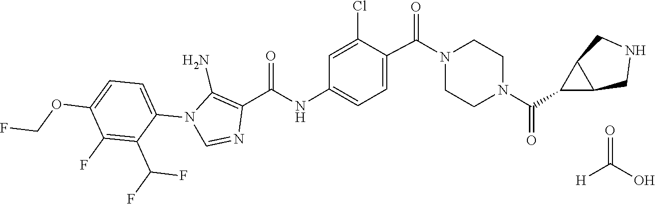

### Step 1: 2-fluoro-1-(fluoromethoxy)-3-methyl-4-nitrobenzene

To a solution of 2-fluoro-3-methyl-4-nitrophenol (1000.0 mg, 5.84 mmol, 1.0 eq) in MeCN (10 mL) were added fluoro (iodo) methane (1401.83 mg, 8.77 mmol, 1.5 eq) and N,N-diisopropylethylamine (2265.65 mg, 17.53 mmol, 3.0 eq). The resulting mixture was stirred at 50° C. for 16 h. The reaction was concentrated to dryness. The residue was taken up in EtOAc (50 ml), and the organics washed with 2×10 mL water followed by 10 mL brine. The organics were then separated and dried (Na₂SO₄) before concentrating to dryness. The crude was then purified by flash column chromatography, eluting with 7% EtOAc in petroleum ether to give 2-fluoro-1-(fluoromethoxy)-3-methyl-4-nitrobenzene (950 mg, 4.68 mmol, 80.0% yield). MS [M+H]⁺: 204.1.

### Step 2: 3-(bromomethyl)-2-fluoro-1-(fluoromethoxy)-4-nitrobenzene

To a solution of 2-fluoro-1-(fluoromethoxy)-3-methyl-4-nitrobenzene (960.0 mg, 4.73 mmol, 1.0 eq) in carbon tetrachloride (15 mL) were added N-bromosuccinimide (1261.65 mg, 7.09 mmol, 1.5 eq) and benzoyl peroxide (114.47 mg, 0.47 mmol, 0.1 eq). The resulting mixture was stirred at 80° C. for 15 h. The reaction was concentrated to dryness and the residue was taken up in EtOAc (5 ml) and the organics washed with 2×5 ml water followed by 5 ml brine. The organics were then separated and dried (Na₂SO₄) before concentrating to dryness. The crude was then purified by flash column chromatography eluting with 7% EtOAc in petroleum ether to give 3-(bromomethyl)-2-fluoro-1-(fluoromethoxy)-4-nitrobenzene (920 mg, 3.26 mmol, 69.0% purity). MS [M+H]⁺: 283.1.

### Step 3: 2-fluoro-3-(fluoromethoxy)-6-nitrobenzaldehyde

To a solution of 3-(bromomethyl)-2-fluoro-1-(fluoromethoxy)-4-nitrobenzene (920.0 mg, 3.26 mmol, 1.0 eq) in MeCN (15 mL) were added N-methylmorpholine N-oxide (917.1 mg, 7.83 mmol, 2.4 eq) and 4A molecular sieves (1980.0 mg). The resulting mixture was stirred at 25° C. for 16 h. The reaction was concentrated to dryness and the residue was taken up in EtOAc (15 ml). The organics washed with 2×5 ml water and 5 ml brine. The organics were then separated and dried (Na₂SO₄) before concentrating to dryness. The crude was then purified by flash column chromatography eluting with 7% EtOAc in petroleum ether to give 2-fluoro-3-(fluoromethoxy)-6-nitrobenzaldehyde (420.0 mg, 1.94 mmol, 57.1% yield). MS [M+H]⁺: 218.1.

### Step 4: 3-(difluoromethyl)-2-fluoro-1-(fluoromethoxy)-4-nitrobenzene

To a solution of 2-fluoro-3-(fluoromethoxy)-6-nitrobenzaldehyde (420.0 mg, 1.93 mmol, 1.0 eq) in bis(2-methoxyethyl)aminosulfur trifluoride (4279.5 mg, 19.34 mmol, 10.0 eq) was added methanol (9.68 mg, 0.19 mmol, 0.1 eq). The resulting mixture was stirred at 50° C. for 3 h. The reaction was concentrated to dryness and the residue was taken up in EtOAc (100 mL) and the organics washed with 2×10 mL water and 10 mL brine. The organics were then separated and dried (Na₂SO₄) before concentrating to dryness. The crude was then purified by flash column chromatography eluting with 7% EtOAc in petroleum ether to give 3-(difluoromethyl)-2-fluoro-1-(fluoromethoxy)-4-nitrobenzene (320.0 mg, 1.34 mmol, 69.2% yield). MS [M+H]⁺: 240.1.

### Step 5: 2-(difluoromethyl)-3-fluoro-4-(fluoromethoxy)aniline

A solution of 3-(difluoromethyl)-2-fluoro-1-(fluoromethoxy)-4-nitrobenzene (320.0 mg, 1.34 mmol, 1.0 eq), iron (373.67 mg, 6.69 mmol, 5.0 eq) and ammonium chloride (357.91 mg, 6.69 mmol, 5.0 eq) in ethanol (5 mL)/water (1 mL) was stirred at 50° C. for 2 h. The solid was filtered out. The crude was then purified by flash column chromatography eluting with 10% EtOAc in petroleum ether to give the 2-(difluoromethyl)-3-fluoro-4-(fluoromethoxy)aniline (210.0 mg, 1.0 mmol, 66.5% yield). MS [M+H]⁺: 210.1.

### Step 6: ethyl 5-amino-1-[2-(difluoromethyl)-3-fluoro-4-(fluoromethoxy)phenyl]imidazole-4-carboxylate

To a solution of ethyl 2-amino-2-cyanoacetate; 4-methylbenzene-1-sulfonic acid (775.45 mg, 2.58 mmol, 3.0 eq) in MeCN (4 mL) were added triethylamine (0.48 mL, 3.4 mmol, 4.0 eq) and triethyl orthoformate (3.81 mL, 22.8 mmol, 26.5 eq). The resulting mixture was stirred at 80° C. for 2 h. The solvent was removed under reduced pressure and the residue was diluted with MeCN (4 mL). Next, 2-(difluoromethyl)-3-fluoro-4-(fluoromethoxy)aniline (180.0 mg, 0.86 mmol, 1.0 eq) was added and the resulting mixture was stirred at 40° C. for 15 h. After concentration under reduced pressure, the residue was purified by flash column chromatography on silica gel (55:45 petroleum ether:EtOAc) to give ethyl 5-amino-1-[2-(difluoromethyl)-3-fluoro-4-(fluoromethoxy)phenyl]imidazole-4-carboxylate (245.0 mg, 0.71 mmol, 66.0% yield). MS [M+H]⁺: 348.2.

### Step 7: 5-amino-1-[2-(difluoromethyl)-3-fluoro-4-(fluoromethoxy)phenyl]imidazole-4-carboxylic acid

To a solution of sodium hydroxide (120.94 mg, 3.02 mmol, 5.0 eq) was added to ethyl 5-amino-1-[2-(difluoromethyl)-3-fluoro-4-(fluoromethoxy)phenyl]imidazole-4-carboxylate (210.0 mg, 0.6 mmol, 1.0 eq) in isopropyl alcohol (1 mL) and water (0.500 mL). The resulting mixture was stirred at 50° C. for 15 h. The mixture was adjusted to pH 5-6 with 2 N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure give 5-amino-1-[2-(difluoromethyl)-3-fluoro-4-(fluoromethoxy)phenyl]imidazole-4-carboxylic acid (120.0 mg, 0.38 mmol, 53.4% yield). MS [M+H]⁺: 320.1.

### Step 8: 5-amino-1-[2-(difluoromethyl)-3-fluoro-4-(fluoromethoxy)phenyl]imidazole-4-carbonyl chloride

A solution of 5-amino-1-[2-(difluoromethyl)-3-fluoro-4-(fluoromethoxy)phenyl]imidazole-4-carboxylic acid (20.0 mg, 0.06 mmol, 1.0 eq) in thionyl chloride (74.54 mg, 0.63 mmol, 10.0 eq) was stirred at 25° C. for 1 h. The resulting solution was concentrated under reduced pressure to give 5-amino-1-[2-(difluoromethyl)-3-fluoro-4-(fluoromethoxy)phenyl]imidazole-4-carbonyl chloride (20.0 mg, 0.06 mmol, 59.8% yield). MS [(M−Cl+OCH₃)+H]⁺: 334.2.

### Step 9: tert-butyl (1R,5S)-6-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate

To a solution of tert-butyl (1R,5S)-6-[4-(4-amino-2-chlorobenzoyl)piperazine-1-carbonyl]-3-azabicyclo[3.1.0]hexane-3-carboxylate (26.59 mg, 0.06 mmol, 1.0 eq) and pyridine (0.05 mL, 0.59 mmol, 10.0 eq) in methylene chloride (2 mL) was added 5-amino-1-[2-(difluoromethyl)-3-fluoro-4-(fluoromethoxy)phenyl]imidazole-4-carbonyl chloride (20.0 mg, 0.06 mmol, 1.0 eq). The resulting mixture was stirred at 25° C. for 3 h. The reaction was concentrated to dryness. The residue was taken up in EtOAc (5 ml) and the organics washed with 2×3 ml water followed by 3 ml brine. The organics were then separated and dried (Na₂SO₄) before concentrating to dryness. The crude was then purified by flash column chromatography eluting with 90% EtOAc in petroleum ether to give tert-butyl (1R,5S)-6-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (87.9 mg, 0.14 mmol, 52.7% yield). MS [M+H]⁺: 750.3.

### Step 10: 5-amino-1-[3-fluoro-4-(fluoromethoxy)-2-methylphenyl]-N-[3-fluoro-4-[4-[(1R,5S)-3-azabicyclo[3.1.0]hexane-6-carbonyl]piperazine-1-carbonyl]phenyl]imidazole-4-carboxamide; 1:1 formic acid

To a solution of tert-butyl (1R,5S)-6-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (50.0 mg, 0.07 mmol, 1.0 eq) in methylene chloride (3 mL) was added hydrogen chloride (4 M in dioxane)(3.33 mL, 13.3 mmol, 200 eq). The reaction mixture was stirred at 25° C. for 1 h. The resulting solution was then concentrated under reduced pressure. The residue was purified by Prep-HPLC (AQ-C18, MeCN— H₂O (0.1% FA)) to give 5-amino-N-[3-chloro-4-[4-[(1R,5S)-3-azabicyclo[3.1.0]hexane-6-carbonyl]piperazine-1-carbonyl]phenyl]-1-[2-(difluoromethyl)-3-fluoro-4-(fluoromethoxy)phenyl]imidazole-4-carboxamide; 1:1 formic acid (13.1 mg, 0.02 mmol, 27.6% yield). MS [M+H]⁺: 650.0

### Compound 125

### 5-amino-N-(3-chloro-4-(4-(piperazine-1-carbonyl)piperazine-1-carbonyl)phenyl)-1-(2-(difluoromethyl)-4-(fluoromethoxy)phenyl)-1H-imidazole-4-carboxamide; 1:1 2,2,2-trifluoroacetate

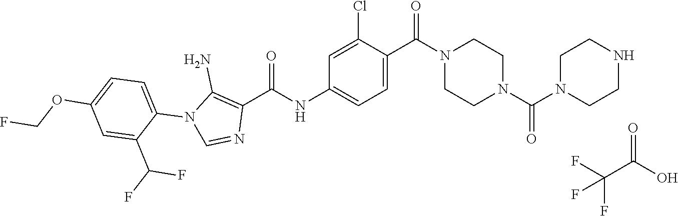

### Step 1: 5-(fluoromethoxy)-2-nitrobenzaldehyde

To a solution of 3-hydroxy-6-nitrobenzaldehyde (2000.0 mg, 11.97 mmol, 1.0 eq) in MeCN (25 mL) were added N,N-diisopropylethylamine (4631.4 mg, 35.9 mmol, 3.0 eq) and fluoro (iodo) methane (1913.95 mg, 11.97 mmol, 1.0 eq). The reaction was stirred at 50° C. for 4 h. The reaction was concentrated to dryness. The residue was taken up in EtOAc (30 mL) and the organics washed with 2×30 mL water followed by 30 mL brine. The organics were then separated and dried (MgSO₄) before concentrating to dryness. The crude was then purified by flash column chromatography eluting with 50% EtOAc in isohexane to afford 5-(fluoromethoxy)-2-nitrobenzaldehyde (1700.0 mg, 8.54 mmol, 52.7% yield). MS [M+H]⁺: 200.1

### Step 2: 2-(difluoromethyl)-4-(fluoromethoxy)-1-nitrobenzene

To a solution of 5-(fluoromethoxy)-2-nitrobenzaldehyde (1650.0 mg, 8.29 mmol, 1.0 eq) in 2-methoxy-N-(2-methoxyethyl)-N-(trifluoro->4-sulfanyl) ethanamine (10.0 mL) was added methanol (30 mL). The reaction was stirred at 50° C. After stirring for 2 h, the reaction mixture was diluted with water (30 mL) and extracted with EtOAc (30 mL). The organics were then separated and dried (MgSO₄) before concentrating under reduced pressure. The crude material was then purified by flash column chromatography eluting with 45% EtOAc in petroleum ether to afford 2-(difluoromethyl)-4-(fluoromethoxy)-1-nitrobenzene (1280.0 mg, 5.79 mmol, 69.9% yield).

### Step 3: 2-(difluoromethyl)-4-(fluoromethoxy)aniline

To a solution of 2-(difluoromethyl)-4-(fluoromethoxy)-1-nitrobenzene (1200.0 mg, 5.43 mmol, 1.0 eq) in methanol (15 mL) was added palladium 10% on carbon (600.0 mg, 5.64 mmol, 1.04 eq). The mixture was stirred 5 h at room temperature under an atmosphere of hydrogen (balloon). The mixture was filtered through diatomaceous earth. The filtrate was concentrated under reduced pressure to afford 2-(difluoromethyl)-4-(fluoromethoxy)aniline (800.0 mg, 4.18 mmol, 94.0% yield) which was used in the next step without further purification. MS [M+H]⁺: 192.1.

### Step 4: ethyl 5-amino-1-[1-[(4-methoxyphenyl)methyl]-3-(trifluoromethyl)pyrazol-4-yl]imidazole-4-carboxylate

To a solution of ethyl 2-amino-2-cyanoacetate; 4-methylbenzene-1-sulfonic acid (2121.09 mg, 7.06 mmol, 3.0 eq) in triethyl orthoformate (17.09 mL, 102.2 mmol, 43.4 eq) was added triethylamine (1429.31 mg, 14.13 mmol, 6.0 eq). The resulting mixture was stirred at 82° C. for 2 h. The solvent was removed under reduced pressure and the residue was diluted with MeCN (20 mL). Next 2-(difluoromethyl)-4-(fluoromethoxy)aniline (450.0 mg, 2.35 mmol, 1.0 eq) was added and the resulting mixture was stirred at 25° C. for 15 h. The reaction was then concentrated to dryness and the residue was taken up in EtOAc (30 mL). The organics were washed with 2×30 mL water followed by 30 mL brine. The organics were then separated and dried (MgSO₄) before concentrating to dryness. The crude was then purified by flash column chromatography eluting with 3% MeOH in methylene chloride to afford ethyl 5-amino-1-[1-[(4-methoxyphenyl)methyl]-3-(trifluoromethyl)pyrazol-4-yl]imidazole-4-carboxylate (400.0 mg, 0.98 mmol, 19.4% yield). MS [M+H]⁺: 330.1

### Step 5: 5-amino-1-[2-(difluoromethyl)-4-(fluoromethoxy)phenyl]imidazole-4-carboxylic acid

To a solution of ethyl 5-amino-1-[2-(difluoromethyl)-4-(fluoromethoxy)phenyl]imidazole-4-carboxylate (380.0 mg, 1.15 mmol, 1.0 eq) in 1-propanol (6 mL) and water (3 mL) was added sodium hydroxide (230.8 mg, 5.77 mmol, 5.0 eq). The reaction was stirred at 25° C. for 16 h, solvent was evaporated. The residue was diluted with water (10 mL), and the solution was extracted with ethyl acetate (2×10 mL). The aqueous layer was acidified to pH 5 with HCl (2 M), and the product was collected by filtration to afford 5-amino-1-[2-(difluoromethyl)-4-(fluoromethoxy)phenyl]imidazole-4-carboxylic acid (245.0 mg, 0.81 mmol, 43.7% yield). MS [M+H]⁺: 302.0

### Step 6: 5-amino-1-(2-(difluoromethyl)-4-(fluoromethoxy)phenyl)-1H-imidazole-4-carbonyl chloride

5-amino-1-[2-(difluoromethyl)-4-(fluoromethoxy)phenyl]imidazole-4-carboxylic acid (50.0 mg, 0.17 mmol, 1.0 eq) was added into thionyl chloride (2 mL) at 20° C. under N₂. The solution was stirred at 20° C. for 1 hour under N₂. The resulting solution was evaporated to dryness with methylene chloride to afford the title compound 5-amino-1-[2-(difluoromethyl)-4-(fluoromethoxy)phenyl]imidazole-4-carbonyl chloride (70.0 mg, 0.22 mmol, 119% yield), which was used without further purification. MS: [M−Cl+OCH₃+H]⁺: 316.1

### Step 7: tert-butyl 4-(4-(4-(5-amino-1-(2-(difluoromethyl)-4-(fluoromethoxy)phenyl)-1H-imidazole-4-carboxamido)-2-chlorobenzoyl)piperazine-1-carbonyl)piperazine-1-carboxylate

To a solution of tert-butyl 4-[4-(4-amino-2-chlorobenzoyl)piperazine-1-carbonyl]piperazine-1-carboxylate (69.28 mg, 0.15 mmol, 0.7 eq) in methylene chloride (3 mL) was added pyridine (0.02 mL, 0.22 mmol, 1.0 eq). The mixture was stirred at 0° C. Next, 5-amino-1-[2-(difluoromethyl)-4-(fluoromethoxy)phenyl]imidazole-4-carbonyl chloride (70.0 mg, 0.22 mmol, 1.0 eq) was added, and the reaction was stirred at 25° C. for 5 h. The reaction was then concentrated to dryness. The residue was taken up in methylene chloride (20 mL) and the organics washed with 2×20 mL water followed by 20 mL brine. The organics were then separated and dried (MgSO₄) before concentrating to dryness. The crude product was purified by Prep-HPLC (Gemini-C18 150×21.2 mm, 5 μm, MeCN-H₂O (0.1% TFA), 20-60) to afford tert-butyl 4-[4-[4-[[5-amino-1-[2-(difluoromethyl)-4-(fluoromethoxy)phenyl]imidazole-4-carbonyl]amino]-2-chlorobenzoyl]piperazine-1-carbonyl]piperazine-1-carboxylate (41.0 mg, 0.06 mmol, 20.5% yield). MS [M+H]⁺: 735.3.

### Step 8: 5-amino-N-(3-chloro-4-(4-(piperazine-1-carbonyl)piperazine-1-carbonyl)phenyl)-1-(2-(difluoromethyl)-4-(fluoromethoxy)phenyl)-1H-imidazole-4-carboxamide 1:1 2,2,2-trifluoroacetate

To a solution of tert-butyl 4-[4-[4-[[5-amino-1-[2-(difluoromethyl)-4-(fluoromethoxy)phenyl]imidazole-4-carbonyl]amino]-2-chlorobenzoyl]piperazine-1-carbonyl]piperazine-1-carboxylate (41.0 mg, 0.06 mmol, 1.0 eq) in methylene chloride (3 mL) was added trifluoroacetic acid (1.0 mL). The reaction was stirred at 25° C. for 3 h and then concentrated to dryness to afford crude product. The crude was then purified by HPLC (Gemini-C18 150×21.2 mm, 5 μm, MeCN-H₂O (0.1% TFA), 15-40) to afford 5-amino-N-[3-chloro-4-[4-(piperazine-1-carbonyl)piperazine-1-carbonyl]phenyl]-1-[2-(difluoromethyl)-4-(fluoromethoxy)phenyl]imidazole-4-carboxamide; 1:1 2,2,2-trifluoroacetic acid (3.6 mg, 0.0 mmol, 9.9% yield). MS [M+H]⁺: 635.1.

### Compound 126

### N-(4-(4-((1R,5S,6r)-3-azabicyclo[3.1.0]hexane-6-carbonyl)piperazine-1-carbonyl)-3-chlorophenyl)-5-amino-1-(2-(difluoromethyl)-4-(fluoromethoxy)phenyl)-1H-imidazole-4-carboxamide 1:1 2,2,2-trifluoroacetate

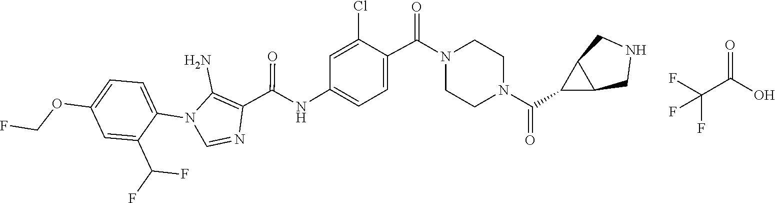

### Step 1: tert-butyl (1R,5S,6r)-6-(4-(4-(5-amino-1-(2-(difluoromethyl)-4-(fluoromethoxy)phenyl)-1H-imidazole-4-carboxamido)-2-chlorobenzoyl)piperazine-1-carbonyl)-3-azabicyclo[3.1.0]hexane-3-carboxylate

To a solution of tert-butyl (1R,5S)-6-[4-(4-amino-2-chlorobenzoyl)piperazine-1-carbonyl]-3-azabicyclo[3.1.0]hexane-3-carboxylate (108.14 mg, 0.24 mmol, 0.7 eq) in methylene chloride (3 mL) were added pyridine (0.56 mL, 6.88 mmol, 20.0 eq) and 5-amino-1-[2-(difluoromethyl)-4-(fluoromethoxy)phenyl]imidazole-4-carbonyl chloride (110.0 mg, 0.34 mmol, 1.0 eq). The reaction was stirred at 25° C. for 5 h and then concentrated to dryness. The residue was taken up in methylene chloride (20 mL) and the organics washed with 2×20 mL water followed by 20 mL brine. The organics were then separated and dried (MgSO₄) before concentrating to dryness. The crude was then purified by flash column chromatography eluting with 10% MeOH in methylene chloride to afford tert-butyl (1R,5S)-6-[4-[4-[[5-amino-1-[2-(difluoromethyl)-4-(fluoromethoxy)phenyl]imidazole-4-carbonyl]amino]-2-chlorobenzoyl]piperazine-1-carbonyl]-3-azabicyclo[3.1.0]hexane-3-carboxylate (100.0 mg, 0.14 mmol, 38.8% yield). MS [M+H]⁺: 732.2.

### Step 2: N-(4-(4-((1R,5S,6r)-3-azabicyclo[3.1.0]hexane-6-carbonyl)piperazine-1-carbonyl)-3-chlorophenyl)-5-amino-1-(2-(difluoromethyl)-4-(fluoromethoxy)phenyl)-1H-imidazole-4-carboxamide 1:1 2,2,2-trifluoroacetate

To a solution of tert-butyl (1R,5S)-6-[4-[4-[[5-amino-1-[2-(difluoromethyl)-4-(fluoromethoxy)phenyl]imidazole-4-carbonyl]amino]-2-chlorobenzoyl]piperazine-1-carbonyl]-3-azabicyclo[3.1.0]hexane-3-carboxylate (100.0 mg, 0.14 mmol, 1.0 eq) in methylene chloride (1 mL) was added trifluoroacetic acid (0.5 mL, 6.5 mmol, 48 eq). The reaction was stirred at 25° C. for 3 h. The reaction was concentrated to afford crude product. The crude product was purified by Prep-HPLC (Gemini C18 150×21.2 mm, 5 μm, MeCN-H₂O (0.1% TFA), 10-40) to afford 5-amino-N-[4-[4-[(1R,5S)-3-azabicyclo[3.1.0]hexane-6-carbonyl]piperazine-1-carbonyl]-3-chlorophenyl]-1-[2-(difluoromethyl)-4-(fluoromethoxy)phenyl]imidazole-4-carboxamide; 1:1 2,2,2-trifluoroacetic acid (35.5 mg, 0.05 mmol, 34.5% yield). MS [M+H]⁺: 632.3.

### Compound 127

### N-[4-[4-(2,3,3a,4,6,6a-hexahydro-1H-pyrrolo[3,4-c]pyrrole-5-carbonyl)piperazine-1-carbonyl]-3-chloro-phenyl]-5-amino-1-[4-(difluoromethoxy)-2,3-difluorophenyl]imidazole-4-carboxamide; 1:1 2,2,2-trifluoroacetic acid

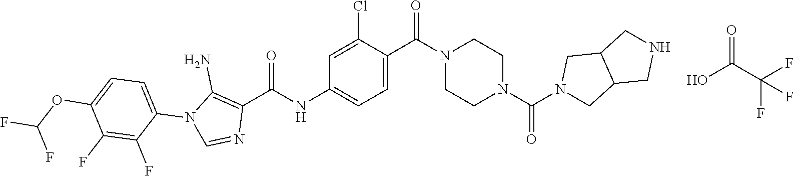 [SMILES: Nc1c(C(=O)Nc2ccc(C(=O)N3CCN(C(=O)N4CC5CNCC5C4)CC3)c(Cl)c2)ncn1-c1ccc(OC(F)F)c(F)c1F ; O=C(O)C(F)(F)F]

### Step 1: tert-butyl 2-[4-(2-chloro-4-nitrobenzoyl)piperazine-1-carbonyl]-1,3,3a,4,6,6a-hexahydropyrrolo[3,4-c]pyrrole-5-carboxylate

A solution of (2-chloro-4-nitrophenyl)-piperazin-1-yl-methanone (500.0 mg, 1.85 mmol, 1.0 eq) and triphosgene (275.09 mg, 0.93 mmol, 0.5 eq) in methylene chloride (5 mL) was stirred at 25° C. for 0.5 h. Next, tert-butyl 2,3,3a,4,6,6a-hexahydro-1H-pyrrolo[3,4-c]pyrrole-5-carboxylate (472.32 mg, 2.22 mmol, 1.2 eq) and triethylamine trihydrofluoride (2988.91 mg, 18.54 mmol, 10.0 eq) were added, the mixture was stirred at 25° C. for 1 h. The resulting solution was partitioned with water and extracted with ethyl acetate. The residue was purified by flash column chromatography on silica gel eluting with 45% EtOAc in petroleum ether to afford tert-butyl 2-[4-(2-chloro-4-nitrobenzoyl)piperazine-1-carbonyl]-1,3,3a,4,6,6a-hexahydropyrrolo[3,4-c]pyrrole-5-carboxylate (690.0 mg, 1.36 mmol, 69.6% yield). MS [(M-Boc)+H]⁺: 408.1.

### Step 2: tert-butyl 2-[4-(4-amino-2-chlorobenzoyl)piperazine-1-carbonyl]-1,3,3a,4,6,6a-hexahydropyrrolo[3,4-c]pyrrole-5-carboxylate

To a solution of tert-butyl 2-[4-(2-chloro-4-nitrobenzoyl)piperazine-1-carbonyl]-1,3,3a,4,6,6a-hexahydropyrrolo[3,4-c]pyrrole-5-carboxylate (680.0 mg, 1.34 mmol, 1.0 eq) in ethanol (10 mL) were added ammonium chloride (358.03 mg, 6.69 mmol, 5.0 eq), iron powder (373.79 mg, 6.69 mmol, 5.0 eq) and water (7 mL). The resulting mixture was stirred at 50° C. for 2 h. The mixture was cooled to room temperature and filtered through a short plug of Celite (200 mg), rinsing the solid support with methanol (30 mL). The solvent was removed under reduced pressure to produce an oil, which was dissolved with ethyl acetate (30 mL), and washed with water, dried (Na₂SO₄) and evaporated to dryness. The residue was purified by flash column chromatography on silica gel eluting with 75% EtOAc in petroleum ether to give tert-butyl 2-[4-(4-amino-2-chlorobenzoyl)piperazine-1-carbonyl]-1,3,3a,4,6,6a-hexahydropyrrolo[3,4-c]pyrrole-5-carboxylate (470.0 mg, 0.98 mmol, 71.2% yield). MS [(M+Na)+H]⁺: 500.0

### Step 3: 5-amino-1-[4-(difluoromethoxy)-2,3-difluoro-phenyl]imidazole-4-carbonyl chloride

A solution of 5-amino-1-[4-(difluoromethoxy)-2,3-difluorophenyl]imidazole-4-carboxylic acid (100.0 mg, 0.33 mmol, 1.0 eq) in thionyl dichloride (38.98 mg, 0.33 mmol, 1.0 eq) was stirred at 25° C. for 1 h. The resulting solution was concentrated under reduced pressure to give 5-amino-1-[4-(difluoromethoxy)-2,3-difluoro-phenyl]imidazole-4-carbonyl chloride (100.0 mg, 0.2 mmol, 90.2% yield). MS [(M−Cl+OCH₃)+H]⁺: 320.0

### Step 4: tert-butyl 2-[4-[4-[[5-amino-1-[4-(difluoromethoxy)-2,3-difluorophenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate

A solution of 5-amino-1-[4-(difluoromethoxy)-2,3-difluorophenyl]imidazole-4-carbonyl chloride (100.0 mg, 0.31 mmol, 1.0 eq) and tert-butyl 2-[4-(4-amino-2-chlorobenzoyl)piperazine-1-carbonyl]-1,3,3a,4,6,6a-hexahydropyrrolo[3,4-c]pyrrole-5-carboxylate (103.39 mg, 0.22 mmol, 0.7 eq) and pyridine (0.5 mL, 6.18 mmol, 20.0 eq) in methylene chloride (1 mL) was stirred at 25° C. for 1 h. The resulting solution was extracted with water and ethyl acetate and then concentrated under reduced pressure at 30° C. The residue was purified by flash column chromatography eluting with 10:1 methylene chloride:MeOH to afford tert-butyl 2-[4-[4-[[5-amino-1-[4-(difluoromethoxy)-2,3-difluoro-phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (70.0 mg, 0.09 mmol, 20.4% yield). MS [(M-Boc)+H]⁺: 665.0

### Step 5: N-[4-[4-(2,3,3a,4,6,6a-hexahydro-1H-pyrrolo[3,4-c]pyrrole-5-carbonyl)piperazine-1-carbonyl]-3-chlorophenyl]-5-amino-1-[4-(difluoromethoxy)-2,3-difluorophenyl]imidazole-4-carboxamide; 1:1 2,2,2-trifluoroacetic acid

A solution of tert-butyl 2-[4-[4-[[5-amino-1-[4-(difluoromethoxy)-2,3-difluorophenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (70.0 mg, 0.09 mmol, 1.0 eq) and trifluoroacetic acid (2.0 mL, 25.96 mmol, 283.76 eq) in methylene chloride (1 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40) to afford N-[4-[4-(2,3,3a,4,6,6a-hexahydro-1H-pyrrolo[3,4-c]pyrrole-5-carbonyl)piperazine-1-carbonyl]-3-chlorophenyl]-5-amino-1-[4-(difluoromethoxy)-2,3-difluoro-phenyl]imidazole-4-carboxamide; 1:1 2,2,2-trifluoroacetic acid (26.0 mg, 0.03 mmol, 42.7% yield). MS [M+H]⁺: 665.2.

### Compound 128

### 5-amino-N-[4-[4-[(1R,5S,6r)-3-azabicyclo[3.1.0]hexane-6-carbonyl]piperazine-1-carbonyl]-3-chlorophenyl]-1-[2,3-difluoro-4-[1-(2-methoxyethyl)-5-methylpyrazol-4-yl]phenyl]imidazole-4-carboxamide; 1:1 2,2,2-trifluoroacetic acid

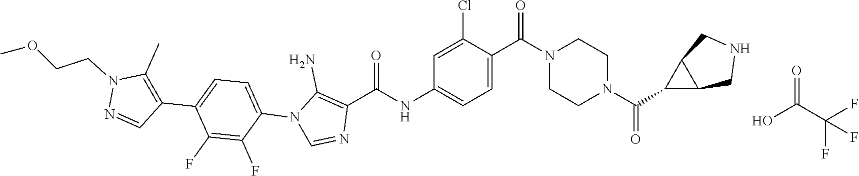 [SMILES: COCCn1ncc(-c2ccc(-n3cnc(C(=O)Nc4ccc(C(=O)N5CCN(C(=O)[C@H]6[C@@H]7CNC[C@@H]76)CC5)c(Cl)c4)c3N)c(F)c2F)c1C ; O=C(O)C(F)(F)F]

### Step 1: 1-(2-methoxyethyl)-5-methyl-4-(4,4,5,5-tetramethyl-1,3,2-dioxaborolan-2-yl)pyrazole

A solution of 5-methyl-4-(4,4,5,5-tetramethyl-1,3,2-dioxaborolan-2-yl)-1H-pyrazole (40.0 g, 192 mmol, 1.0 eq), potassium carbonate (53.06 g, 384.5 mmol, 2.0 eq), and 2-bromoethyl methyl ether (36.13 mL, 384.5 mmol, 2.0 eq) in DMF (200 mL) was stirred at 110° C. for 12 h. The reaction was concentrated to dryness and the residue was taken up in EtOAc (500 mL) and the organics washed with 2×500 mL water followed by 500 mL brine. The organics were then separated and dried (MgSO₄) before concentrating to dryness. The crude was then purified by flash column chromatography eluting with 30% EtOAc in isohexane to afford 1-(2-methoxyethyl)-5-methyl-4-(4,4,5,5-tetramethyl-1,3,2-dioxaborolan-2-yl)pyrazole (33.0 g, 123.99 mmol, 58.0% yield). MS [M+H]⁺: 267.1.

### Step 2: 2,3-difluoro-4-[1-(2-methoxyethyl)-5-methylpyrazol-4-yl]aniline

To a solution of 1-(2-methoxyethyl)-3-methyl-4-(4,4,5,5-tetramethyl-1,3,2-dioxaborolan-2-yl)pyrazole (1407.47 mg, 5.29 mmol, 0.55 eq) in 1,4-dioxane (80 mL) were added 4-bromo-2,3-difluoroaniline (2000.0 mg, 9.62 mmol, 1.0 eq), [1,1′-bis(diphenylphosphino)ferrocene]dichloropalladium (II) (703.57 mg, 0.96 mmol, 0.1 eq) and potassium carbonate (2657.81 mg, 19.23 mmol, 2.0 eq) under N₂ atmosphere. The resulting mixture was stirred at 110° C. for 15 h before concentration under reduced pressure. The residue was then diluted with EtOAc (50 mL), washed with H₂O (3×25 mL), dried over Na₂SO₄ and concentrated under reduced pressure. The residue was purified by flash column chromatography (65:35 petroleum ether:EtOAc) to afford 2,3-difluoro-4-[1-(2-methoxyethyl)-3-methyl-pyrazol-4-yl]aniline (1790.0 mg, 6.7 mmol, 34.8% yield)/2,3-difluoro-4-[1-(2-methoxyethyl)-5-methyl-pyrazol-4-yl]aniline (1790.0 mg, 6.7 mmol, 34.8% yield). MS [M+H]⁺: 268.1.

### Step 3: ethyl 5-amino-1-[2,3-difluoro-4-[1-(2-methoxyethyl)-5-methyl-pyrazol-4-yl]phenyl]imidazole-4-carboxylate

A mixture of ethyl 2-amino-2-cyanoacetate; 4-methylbenzene-1-sulfonic acid (3.02 g, 10.05 mmol, 3.0 eq), triethylamine (2.8 mL, 20.09 mmol, 6.0 eq) and triethyl orthoformate (4.96 g, 33.49 mmol, 10.0 eq) was stirred at 80° C. for 2 h. The solvent was removed under reduced pressure and the residue was diluted with MeCN (90 mL). Next 2,3-difluoro-4-[1-(2-methoxyethyl)-3-methyl-pyrazol-4-yl]aniline;2,3-difluoro-4-[1-(2-methoxyethyl)-5-methylpyrazol-4-yl]aniline (1.79 g, 3.35 mmol, 1.0 eq) was added and the resulting mixture was stirred at 40° C. for 15 h. After concentration under reduced pressure, the residue was purified by flash column chromatography (30:70 petroleum ether:EtOAc) to afford ethyl 5-amino-1-[2,3-difluoro-4-[1-(2-methoxyethyl)-5-methylpyrazol-4-yl]phenyl]imidazole-4-carboxylate (430.0 mg, 1.06 mmol, 31.7% yield). MS [M+H]⁺: 406.1.

### Step 4: 5-amino-1-[2,3-difluoro-4-[1-(2-methoxyethyl)-5-methylpyrazol-4-yl]phenyl]imidazole-4-carboxylic acid

A solution of ethyl 5-amino-1-[2,3-difluoro-4-[1-(2-methoxyethyl)-5-methylpyrazol-4-yl]phenyl]imidazole-4-carboxylate (410.0 mg, 1.01 mmol, 1.0 eq), sodium hydroxide (202.25 mg, 5.06 mmol, 5.0 eq) in isopropyl alcohol (6 mL) and water (2 mL) was stirred at 50° C. for 6 h. The residue was diluted with 10 mL of water, then adjusted to pH 6˜7 with HCl (2M) and extracted with 3×10 ml of EtOAc. The organic layers were combined and concentrated under reduced pressure to give 5-amino-1-[2,3-difluoro-4-[1-(2-methoxyethyl)-5-methylpyrazol-4-yl]phenyl]imidazole-4-carboxylic acid (310.0 mg, 0.82 mmol, 76.4% yield). MS [M+H]⁺: 378.1

### Step 5: 5-amino-1-[2,3-difluoro-4-[1-(2-methoxyethyl)-5-methylpyrazol-4-yl]phenyl]imidazole-4-carbonyl chloride

A solution of 5-amino-1-[2,3-difluoro-4-[1-(2-methoxyethyl)-5-methylpyrazol-4-yl]phenyl]imidazole-4-carboxylic acid (50.0 mg, 0.13 mmol, 1.0 eq) in thionyl chloride (1.5 mL, 21 mmol, 160 eq) was stirred at 25° C. for 1 h. The resulting solution was concentrated under reduced pressure to give 5-amino-1-[2,3-difluoro-4-[1-(2-methoxyethyl)-5-methylpyrazol-4-yl]phenyl]imidazole-4-carbonyl chloride (50.0 mg, 0.13 mmol, 95.3% yield). MS [M-Cl+OMe+H]⁺: 392.1

### Step 6: tert-butyl (1R,5S)-6-[4-[4-[[5-amino-1-[2,3-difluoro-4-[1-(2-methoxyethyl)-5-methyl-pyrazol-4-yl]phenyl]imidazole-4-carbonyl]amino]-2-chlorobenzoyl]piperazine-1-carbonyl]-3-azabicyclo[3.1.0]hexane-3-carboxylate

A solution of tert-butyl (1R,5S)-6-[4-(4-amino-2-chlorobenzoyl)piperazine-1-carbonyl]-3-azabicyclo[3.1.0]hexane-3-carboxylate (39.7 mg, 0.09 mmol, 0.7 eq) and pyridine (0.2 mL, 2.53 mmol, 20.0 eq) in methylene chloride (2 mL) was stirred at 25° C. A solution of 5-amino-1-[2,3-difluoro-4-[1-(2-methoxyethyl)-5-methyl-pyrazol-4-yl]phenyl]imidazole-4-carbonyl chloride (50.0 mg, 0.13 mmol, 1.0 eq) in methylene chloride (2 mL) was added, and the temperature was increased to 25° C. for 1 h. The resulting solution was concentrated under reduced pressure. The residue was applied on a silica gel column and eluted with 10:1 methylene chloride:MeOH to give tert-butyl (1R,5S)-6-[4-[4-[[5-amino-1-[2,3-difluoro-4-[1-(2-methoxyethyl)-5-methylpyrazol-4-yl]phenyl]imidazole-4-carbonyl]amino]-2-chlorobenzoyl]piperazine-1-carbonyl]-3-azabicyclo[3.1.0]hexane-3-carboxylate (60.0 mg, 0.07 mmol, 37.0% yield). MS [M+H]⁺: 808.1

### Step 7: 5-amino-N-[4-[4-[(1R,5S)-3-azabicyclo[3.1.0]hexane-6-carbonyl]piperazine-1-carbonyl]-3-chlorophenyl]-1-[2,3-difluoro-4-[1-(2-methoxyethyl)-5-methylpyrazol-4-yl]phenyl]imidazole-4-carboxamide; 1:1 2,2,2-trifluoroacetic acid

A solution of tert-butyl (1R,5S)-6-[4-[4-[[5-amino-1-[2,3-difluoro-4-[1-(2-methoxyethyl)-5-methylpyrazol-4-yl]phenyl]imidazole-4-carbonyl]amino]-2-chlorobenzoyl]piperazine-1-carbonyl]-3-azabicyclo[3.1.0]hexane-3-carboxylate (60.0 mg, 0.07 mmol, 1.0 eq), trifluoracetic acid (1.0 mL, 13 mmol, 170 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40) to afford 5-amino-N-[4-[4-[(1R,5S)-3-azabicyclo[3.1.0]hexane-6-carbonyl]piperazine-1-carbonyl]-3-chlorophenyl]-1-[2,3-difluoro-4-[1-(2-methoxyethyl)-5-methylpyrazol-4-yl]phenyl]imidazole-4-carboxamide; 1:2,2,2-trifluoroacetic acid (16.3 mg, 0.02 mmol, 26.1% yield). MS [M+H]⁺: 707.9

### Compound 129

### 5-amino-1-[2,3-difluoro-4-(fluoromethoxy)phenyl]-N-[3-fluoro-4-[4-[(3aS,6aR)-2,3,3a,4,6,6a-hexahydro-1H-pyrrolo[3,4-c]pyrrole-5-carbonyl]piperazine-1-carbonyl]phenyl]imidazole-4-carboxamide; 1:1 2,2,2-trifluoroacetic acid

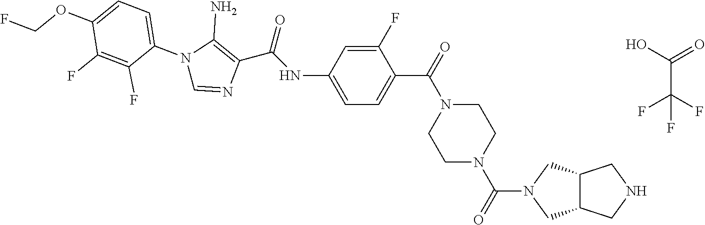 [SMILES: Nc1c(C(=O)Nc2ccc(C(=O)N3CCN(C(=O)N4C[C@H]5CNC[C@H]5C4)CC3)c(F)c2)ncn1-c1ccc(OCF)c(F)c1F ; O=C(O)C(F)(F)F]

### Step 1: tert-butyl (3aR,6aS)-2-[4-(2-fluoro-4-nitrobenzoyl)piperazine-1-carbonyl]-1,3,3a,4,6,6a-hexahydropyrrolo[3,4-c]pyrrole-5-carboxylate

A solution of (2-fluoro-4-nitrophenyl)-piperazin-1-yl-methanone (400.0 mg, 1.58 mmol, 1.0 eq) and triphosgene (234.37 mg, 0.79 mmol, 0.5 eq) in methylene chloride (5 mL) was stirred at 25° C. for 0.5 h. Next were added triethylamine (2.2 mL, 15.8 mmol, 10.0 eq) and tert-butyl (3aR,6aS)-hexahydropyrrolo[3,4-c]pyrrole-2 (1H)-carboxylate (402.4 mg, 1.9 mmol, 1.2 eq). The mixture was stirred at 25° C. for 1 h. The reaction was concentrated to dryness. The crude was purified by flash column chromatography (70:30 petroleum ether:EtOAc) to yield tert-butyl (3aR,6aS)-2-[4-(2-fluoro-4-nitrobenzoyl)piperazine-1-carbonyl]-1,3,3a,4,6,6a-hexahydropyrrolo[3,4-c]pyrrole-5-carboxylate (534.0 mg, 1.09 mmol, 41.3% yield). MS [(M-Boc)+H]⁺: 392.2

### Step 2: tert-butyl (3aR,6aS)-2-[4-(4-amino-2-fluorobenzoyl)piperazine-1-carbonyl]-1,3,3a,4,6,6a-hexahydropyrrolo[3,4-c]pyrrole-5-carboxylate

A mixture of tert-butyl (3aR,6aS)-2-[4-(2-fluoro-4-nitrobenzoyl)piperazine-1-carbonyl]-1,3,3a,4,6,6a-hexahydropyrrolo[3,4-c]pyrrole-5-carboxylate (534.0 mg, 1.09 mmol, 1.0 eq), ammonium chloride (290.57 mg, 5.43 mmol, 5.0 eq) and iron (303.36 mg, 5.43 mmol, 5.0 eq) in ethanol (10 mL) and water (2 mL) was stirred at 50° C. for 2 h. Iron was removed by filtration and then the resulting solution was concentrated under reduced pressure. The crude was extracted by water and ethyl acetate andconcentrated under reduced pressure to afford tert-butyl (3aR,6aS)-2-[4-(4-amino-2-fluorobenzoyl)piperazine-1-carbonyl]-1,3,3a,4,6,6a-hexahydropyrrolo[3,4-c]pyrrole-5-carboxylate (450.0 mg, 0.98 mmol, 82.6% yield). MS [(M−Boc)+H]⁺: 362.1

### Step 3: tert-butyl (3aR,6aS)-2-[4-[4-[[5-amino-1-[2,3-difluoro-4-(fluoromethoxy)phenyl]imidazole-4-carbonyl]amino]-2-fluorobenzoyl]piperazine-1-carbonyl]-1,3,3a,4,6,6a-hexahydropyrrolo[3,4-c]pyrrole-5-carboxylate

A mixture of tert-butyl (3aR,6aS)-2-[4-(4-amino-2-fluorobenzoyl)piperazine-1-carbonyl]-1,3,3a,4,6,6a-hexahydropyrrolo[3,4-c]pyrrole-5-carboxylate (105.7 mg, 0.23 mmol, 1.0 eq) and pyridine (0.19 mL, 2.29 mmol, 10.0 eq) in methylene chloride (2 mL) were added 5-amino-1-[2,3-difluoro-4-(fluoromethoxy)phenyl]imidazole-4-carbonyl chloride (70.0 mg, 0.23 mmol, 1.0 eq) was stirred at 25° C. for 1 h. The reaction was concentrated to dryness and the residue was taken up in EtOAc (5 mL) and the organics washed with 2×3 mL water and 3 mL brine. The organics were then separated and dried (Na₂SO₄) before concentrating to dryness. The crude was then purified by flash column chromatography eluting with 90% EtOAc in petroleum ether to give tert-butyl (3aR,6aS)-2-[4-[4-[[5-amino-1-[2,3-difluoro-4-(fluoromethoxy)phenyl]imidazole-4-carbonyl]amino]-2-fluorobenzoyl]piperazine-1-carbonyl]-1,3,3a,4,6,6a-hexahydropyrrolo[3,4-c]pyrrole-5-carboxylate (130.0 mg, 0.18 mmol, 77.7% yield). MS [(M−Boc)+H]⁺: 631.0

### Step 4: 5-amino-1-[2,3-difluoro-4-(fluoromethoxy)phenyl]-N-[3-fluoro-4-[4-[(3aS,6aR)-2,3,3a,4,6,6a-hexahydro-1H-pyrrolo[3,4-c]pyrrole-5-carbonyl]piperazine-1-carbonyl]phenyl]imidazole-4-carboxamide; 1:1 2,2,2-trifluoroacetic acid

A solution of tert-butyl (3aR,6aS)-2-[4-[4-[[5-amino-1-[2,3-difluoro-4-(fluoromethoxy)phenyl]imidazole-4-carbonyl]amino]-2-fluorobenzoyl]piperazine-1-carbonyl]-1,3,3a,4,6,6a-hexahydropyrrolo[3,4-c]pyrrole-5-carboxylate (130.0 mg, 0.18 mmol, 1.0 eq) and trifluoroacetic acid (1.0 mL, 12.98 mmol, 72.96 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA), 15-40) to give 5-amino-1-[2,3-difluoro-4-(fluoromethoxy)phenyl]-N-[3-fluoro-4-[4-[(3aS,6aR)-2,3,3a,4,6,6a-hexahydro-1H-pyrrolo[3,4-c]pyrrole-5-carbonyl]piperazine-1-carbonyl]phenyl]imidazole-4-carboxamide; 1:1 2,2,2-trifluoroacetic acid (46.4 mg, 0.06 mmol, 35.0% yield). MS [M+H]⁺: 631.3.

### Compound 130

### 5-amino-1-[2,3-difluoro-4-(fluoromethoxy)phenyl]-N-[3-fluoro-4-[4-(piperazine-1-carbonyl)piperazine-1-carbonyl]phenyl]imidazole-4-carboxamide; 1:1 2,2,2-trifluoroacetic acid

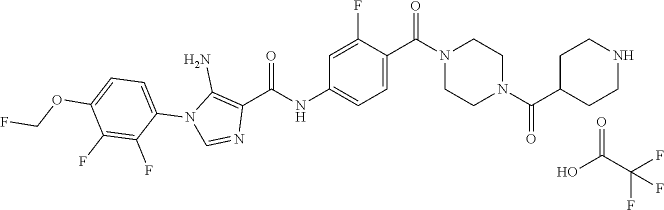

### Step 1: tert-butyl 4-[4-(2-fluoro-4-nitrobenzoyl)piperazine-1-carbonyl]piperazine-1-carboxylate

To a solution of (2-fluoro-4-nitrophenyl)-piperazin-1-yl-methanone (400.0 mg, 1.58 mmol, 1.0 eq) in methylene chloride (2 mL) was added triphosgene (234.37 mg, 0.79 mmol, 0.5 eq) at 25° C. After stirring for 0.5 h, triethylamine (2.2 mL, 15.8 mmol, 10.0 eq) and 1-BOC-piperazine (353.04 mg, 1.9 mmol, 1.2 eq) were added, and the resulting mixture was stirred at 25° C. for 1 h. The resulting solution was concentrated under reduced pressure. The residue was applied on a silica gel column and eluted with 1:20 MeOH:methylene chloride to give tert-butyl 4-[4-(2-fluoro-4-nitrobenzoyl)piperazine-1-carbonyl]piperazine-1-carboxylate (470.0 mg, 1.01 mmol, 55% yield). MS [M+Na]⁺: 488.0

### Step 2: tert-butyl 4-[4-(4-amino-2-fluorobenzoyl)piperazine-1-carbonyl]piperazine-1-carboxylate

A solution of tert-butyl 4-[4-(2-fluoro-4-nitrobenzoyl)piperazine-1-carbonyl]piperazine-1-carboxylate (450.0 mg, 0.97 mmol, 1.0 eq), iron powder (269.94 mg, 4.83 mmol, 5.0 eq) and ammonium chloride (258.56 mg, 4.83 mmol, 5.0 eq) in ethanol (5 mL) and water (2 mL) was stirred at 50° C. for 3 h. The solid was filtered off. The crude was purified by flash column chromatography eluting with 40% EtOAc in petroleum ether to give tert-butyl 4-[4-(4-amino-2-fluorobenzoyl)piperazine-1-carbonyl]piperazine-1-carboxylate (264.0 mg, 0.61 mmol, 59.6% yield). MS [M+H]⁺: 436.3.

### Step 3: tert-butyl 4-[4-[4-[[5-amino-1-[2,3-difluoro-4-(fluoromethoxy)phenyl]imidazole-4-carbonyl]amino]-3-fluorobenzoyl]piperazine-1-carbonyl]piperazine-1-carboxylate

A solution of 5-amino-1-[2,3-difluoro-4-(fluoromethoxy)phenyl]imidazole-4-carbonyl chloride (50.0 mg, 0.16 mmol, 1.0 eq), tert-butyl 4-[4-(4-amino-3-fluorobenzoyl)piperazine-1-carbonyl]piperazine-1-carboxylate (49.87 mg, 0.11 mmol, 0.7 eq) and pyridine (0.26 mL, 3.27 mmol, 20.0 eq) in methylene chloride (1 mL) was stirred at 25° C. for 1 h. After concentration under reduced pressure, the residue was purified by flash column chromatography on silica gel to give tert-butyl 4-[4-[4-[[5-amino-1-[2,3-difluoro-4-(fluoromethoxy)phenyl]imidazole-4-carbonyl]amino]-3-fluorobenzoyl]piperazine-1-carbonyl]piperazine-1-carboxylate (85.0 mg, 0.12 mmol, 65.6% yield). MS [M+H]⁺: 705.0

### Step 4: 5-amino-1-[2,3-difluoro-4-(fluoromethoxy)phenyl]-N-[3-fluoro-4-[4-(piperazine-1-carbonyl)piperazine-1-carbonyl]phenyl]imidazole-4-carboxamide; 1:1 2,2,2-trifluoroacetic acid

A solution of tert-butyl 4-[4-[4-[[5-amino-1-[2,3-difluoro-4-(fluoromethoxy)phenyl]imidazole-4-carbonyl]amino]-2-fluorobenzoyl]piperazine-1-carbonyl]piperazine-1-carboxylate (80.0 mg, 0.11 mmol, 1.0 eq) and trifluoroacetic acid (1.0 mL, 13 mmol, 110 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40) to afford 5-amino-1-[2,3-difluoro-4-(fluoromethoxy)phenyl]-N-[3-fluoro-4-[4-(piperazine-1-carbonyl)piperazine-1-carbonyl]phenyl]imidazole-4-carboxamide; 1:1 2,2,2-trifluoroacetic acid (32.1 mg, 0.04 mmol, 39% yield). MS [M+H]⁺: 605.3.

### Compound 131

### 5-amino-N-[4-[4-[(1R,5S,6r)-3-azabicyclo[3.1.0]hexane-6-carbonyl]piperazine-1-carbonyl]-3-chlorophenyl]-1-[1-(cyclobutylmethyl)-3-(trifluoromethyl)pyrazol-4-yl]imidazole-4-carboxamide

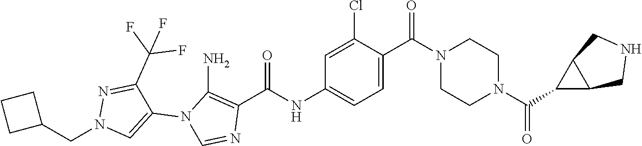 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2CCC2)nc1C(F)(F)F]

### Step 1: 1-[(4-methoxyphenyl)methyl]-4-nitro-3-(trifluoromethyl)pyrazole

To a solution of 4-nitro-3-(trifluoromethyl)-1H-pyrazole (11.0 g, 60.75 mmol, 1.0 eq) in DMF (100 mL) were added potassium carbonate (25.19 g, 182.25 mmol, 3.0 eq) and 4-methoxybenzylchloride (9.88 mL, 72.9 mmol, 1.2 eq) under N₂ atmosphere. The vessel was sealed, and the mixture was stirred at 25° C. for 15 h. The resulting solution was diluted with 200 ml of water, then extracted with 3×200 mL of EtOAc. The organic layers were combined, washed with brine, dried and concentrated under reduced pressure. The residue was applied on a silica gel column and eluted with petroleum ether/EtOAc (10:1-100) to give 1-[(4-methoxyphenyl)methyl]-4-nitro-3-(trifluoromethyl)pyrazole (16.5 g, 54.8 mmol, 90.2% yield).

### Step 2: 1-[(4-methoxyphenyl)methyl]-3-(trifluoromethyl)pyrazol-4-amine

A solution of 1-[(4-methoxyphenyl)methyl]-4-nitro-3-(trifluoromethyl)pyrazole (16.5 g, 54.78 mmol, 1.0 eq), ammonium chloride (14.65 g, 273.89 mmol, 5.0 eq) and Fe (15.34 g, 273.9 mmol, 5.0 eq) in ethanol (100 mL) water (100 mL) was heated to 60° C. for 4 h. The mixture was cooled to room temperature and filtered through a short plug of Celite (1 g), rinsing the solid support with methanol (100 mL), the solvent was removed under reduced pressure to produce anan oil, which was dissolved with ethyl acetate (200 mL), and washed with water, dried (Na₂SO₄) and evaporated to dryness to afford 1-[(4-methoxyphenyl)methyl]-3-(trifluoromethyl)pyrazol-4-amine (12.6 g, 46.4 mmol, 78.9% yield). MS [M+H]⁺: 272.1.

### Step 3: ethyl 5-amino-1-[1-[(4-methoxyphenyl)methyl]-3-(trifluoromethyl)pyrazol-4-yl]imidazole-4-carboxylate

A mixture of ethyl 2-amino-2-cyanoacetate; 4-methylbenzene-1-sulfonic acid (27.9 g, 92.9 mmol, 2.0 eq) triethylamine (25.9 mL, 186 mmol, 4.0 eq) and triethyl orthoformate (68.84 g, 464.5 mmol, 10.0 eq) was stirred at 80° C. for 2 h. The solvent was removed under reduced pressure and the residue was diluted with MeCN (90 mL), then 1-[(4-methoxyphenyl)methyl]-3-(trifluoromethyl)pyrazol-4-amine (12.6 g, 46.4 mmol, 1.0 eq) was added and the resulting mixture was stirred at 40° C. for 15 h. After concentration under reduced pressure, the residue was purified by flash column chromatography on silica gel (50:50 petroleum ether:EtOAc) to give ethyl 5-amino-1-[1-[(4-methoxyphenyl)methyl]-3-(trifluoromethyl)pyrazol-4-yl]imidazole-4-carboxylate (10.5 g, 25.6 mmol, 55.2% yield). MS [M+H]⁺: 410.1

### Step 4: 5-amino-1-[1-[(4-methoxyphenyl)methyl]-3-(trifluoromethyl)pyrazol-4-yl]imidazole-4-carboxylic acid

A solution of ethyl 5-amino-1-[1-[(4-methoxyphenyl)methyl]-3-(trifluoromethyl)pyrazol-4-yl]imidazole-4-carboxylate (1.0 g, 2.4 mmol, 1.0 eq), lithium hydroxide (0.23 mL, 24 mmol, 10 eq) in the methanol (10 mL) and water (4 mL) was stirred at 70° C. for 6 h. The residue was diluted with 20 ml of water, then adjusted to pH 5˜6 with HCl (2 M) and extracted with 3×20 mL of EtOAc. The organic layers were combined and concentrated under reduced pressure to give 5-amino-1-[1-[(4-methoxyphenyl)methyl]-3-(trifluoromethyl)pyrazol-4-yl]imidazole-4-carboxylic acid (520.0 mg, 1.36 mmol, 53.0% yield). MS [M+H]⁺: 382.0

### Step 5: 5-amino-1-[1-[(4-methoxyphenyl)methyl]-3-(trifluoromethyl)pyrazol-4-yl]imidazole-4-carbonyl chloride

A solution of 5-amino-1-[1-[(4-methoxyphenyl)methyl]-3-(trifluoromethyl)pyrazol-4-yl]imidazole-4-carboxylic acid (300.0 mg, 0.79 mmol, 1.0 eq) in thionyl chloride (3.0 mL, 41.3 mmol, 52.5 eq) was stirred at 25° C. for 1 h. The resulting solution was concentrated under reduced pressure to give 5-amino-1-[1-[(4-methoxyphenyl)methyl]-3-(trifluoromethyl)pyrazol-4-yl]imidazole-4-carbonyl chloride (300.0 mg, 0.75 mmol, 85.8% yield). MS [M−Cl+OMe+H]⁺: 396.0

### Step 6: tert-butyl (1R,5S)-6-[4-[4-[[5-amino-1-[1-[(4-methoxyphenyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of tert-butyl (1R,5S)-6-[4-(4-amino-2-chlorobenzoyl)piperazine-1-carbonyl]-3-azabicyclo[3.1.0]hexane-3-carboxylate (336.92 mg, 0.75 mmol, 1.0 eq) and pyridine (1.21 mL, 15.01 mmol, 20.0 eq) in methylene chloride (3 mL) was stirred at 25° C. This was followed by the addition of 5-amino-1-[1-[(4-methoxyphenyl)methyl]-3-(trifluoromethyl)pyrazol-4-yl]imidazole-4-carbonyl chloride (300.0 mg, 0.75 mmol, 1.0 eq) in methylene chloride (3 mL), and the temperature was increased to 25° C. for 1 h. The resulting solution was concentrated under reduced pressure. The residue was applied on a silica gel column and eluted with 10:1 methylene chloride:MeOH to give tert-butyl (1R,5S)-6-[4-[4-[[5-amino-1-[1-[(4-methoxyphenyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (430.0 mg, 0.53 mmol, 53.6% yield). MS [M+H]⁺: 812.3

### Step 7: 5-amino-N-[4-[4-[(1R,5S)-3-azabicyclo[3.1.0]hexane-6-carbonyl]piperazine-1-carbonyl]-3-chlorophenyl]-1-[3-(trifluoromethyl)-1H-pyrazol-4-yl]imidazole-4-carboxamide

A solution of tert-butyl (1R,5S)-6-[4-[4-[[5-amino-1-[1-[(4-methoxyphenyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (430.0 mg, 0.53 mmol, 1.0 eq) in the trifluoroacetic acid (3.0 mL, 39 mmol, 74 eq) was stirred at 70° C. for 8 h. The reaction was concentrated to dryness to give 5-amino-N-[4-[4-[(1R,5S)-3-azabicyclo[3.1.0]hexane-6-carbonyl]piperazine-1-carbonyl]-3-chlorophenyl]-1-[3-(trifluoromethyl)-1H-pyrazol-4-yl]imidazole-4-carboxamide (220.0 mg, 0.37 mmol, 49.14% yield). MS [M+H]⁺: 593.0

### Step 8: tert-butyl (1R,5S)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate

To a solution of 5-amino-N-[4-[4-[(1R,5S)-3-azabicyclo[3.1.0]hexane-6-carbonyl]piperazine-1-carbonyl]-3-chlorophenyl]-1-[3-(trifluoromethyl)-1H-pyrazol-4-yl]imidazole-4-carboxamide (200.0 mg, 0.34 mmol, 1.0 eq) in the THF (5 mL) and water (1 mL) was added tert-butoxycarbonyl anhydride (73.74 mg, 0.34 mmol, 1.0 eq) and sodium hydrogen carbonate (85.15 mg, 1.01 mmol, 3.0 eq). The mixture was stirred at 25° C. for 1 h. The resulting solution was diluted with 10 ml of water and extracted with 3×10 mL of ethyl acetate. The organic layers were combined, concentrated to dryness to afford crude product. The crude was then purified by flash column chromatography eluting with 10:1 methylene chloride:MeOH to give tert-butyl (1R,5S)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (180.0 mg, 0.26 mmol, 77% yield). MS [M−t−Bu+H]⁺: 636.1

### Step 9: tert-butyl (1R,5S)-6-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl (1R,5S)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (100.0 mg, 0.14 mmol, 1.0 eq) in DMF (3 mL) were added (bromomethyl)cyclobutane (25.84 mg, 0.17 mmol, 1.2 eq) and potassium carbonate (59.91 mg, 0.43 mmol, 3.0 eq). The mixture was stirred at 25° C. for 16 h. The resulting solution was diluted with 5.0 ml of water and extracted with 3×5 mL of ethyl acetate. The organic layers were combined, concentrated to dryness to afford crude product. The crude was then purified by flash column chromatography eluting with 10:1 methylene chloride:MeOH to give tert-butyl (1R,5S)-6-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (70.0 mg, 0.09 mmol, 63.7% yield). MS [M−t−Bu+H]⁺: 704.2

### Step 10: 5-amino-N-[4-[4-[(1R,5S)-3-azabicyclo[3.1.0]hexane-6-carbonyl]piperazine-1-carbonyl]-3-chlorophenyl]-1-[1-(cyclobutylmethyl)-3-(trifluoromethyl)pyrazol-4-yl]imidazole-4-carboxamide

A solution of tert-butyl (1R,5S)-6-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (60.0 mg, 0.08 mmol, 1.0 eq), trifluoroacetic acid (1.0 mL, 13 mmol, 160 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% NH₃·H₂O), 30-40) to afford 5-amino-N-[4-[4-[(1R,5S)-3-azabicyclo[3.1.0]hexane-6-carbonyl]piperazine-1-carbonyl]-3-chlorophenyl]-1-[1-(cyclobutylmethyl)-3-(trifluoromethyl)pyrazol-4-yl]imidazole-4-carboxamide; (17.3 mg, 0.02 mmol, 32.7% yield). MS [M+H]⁺: 660.4

### Compound 132

### 5-amino-N-[3-chloro-4-[4-(7-methyl-9-oxa-3,7-diazabicyclo[3.3.1]nonane-3-carbonyl)piperazine-1-carbonyl]phenyl]-1-[2,3-difluoro-4-(fluoromethoxy)phenyl]imidazole-4-carboxamide; 1:1 2,2,2-trifluoroacetic acid

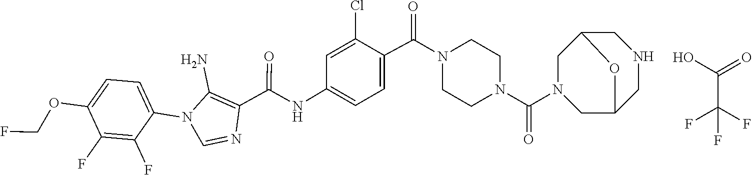 [SMILES: Nc1c(C(=O)Nc2ccc(C(=O)N3CCN(C(=O)N4CC5CNCC(C4)O5)CC3)c(Cl)c2)ncn1-c1ccc(OCF)c(F)c1F ; O=C(O)C(F)(F)F]

### Compound 133

### 5-amino-N-[3-chloro-4-[4-(9-oxa-3,7-diazabicyclo[3.3.1]nonane-3-carbonyl)piperazine-1-carbonyl]phenyl]-1-[2,3-difluoro-4-(fluoromethoxy)phenyl]imidazole-4-carboxamide; 1:1 2,2,2-trifluoroacetic acid

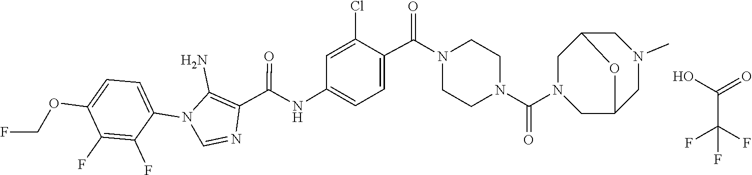

### Step 1: tert-butyl 7-[4-(2-chloro-4-nitrobenzoyl)piperazine-1-carbonyl]-9-oxa-3,7-diazabicyclo[3.3.1]nonane-3-carboxylate

To a solution of (2-chloro-4-nitro-phenyl)-piperazin-1-yl-methanone (300.0 mg, 1.11 mmol, 1.0 eq) in methylene chloride (2 mL) was added triphosgene (198.23 mg, 0.67 mmol, 0.6 eq) at 25° C. After stirring for 0.5 h, triethylamine (1123.55 mg, 11.12 mmol, 10.0 eq) and tert-butyl 9-oxa-3,7-diazabicyclo[3.3.1]nonane-3-carboxylate (253.96 mg, 1.11 mmol, 1.0 eq) were added, and the resulting mixture was stirred at 25° C. for 1 h. The resulting solution was concentrated under reduced pressure. The residue was applied on a silica gel column and eluted with 1:20 MeOH:methylene chloride to give tert-butyl 7-[4-(2-chloro-4-nitrobenzoyl)piperazine-1-carbonyl]-9-oxa-3,7-diazabicyclo[3.3.1]nonane-3-carboxylate (450.0 mg, 0.86 mmol, 77.2% yield). MS [(M−Boc+H]⁺: 424.2.

### Step 2: tert-butyl 7-[4-(4-amino-2-chlorobenzoyl)piperazine-1-carbonyl]-9-oxa-3,7-diazabicyclo[3.3.1]nonane-3-carboxylate

A solution of tert-butyl 7-[4-(2-chloro-4-nitrobenzoyl)piperazine-1-carbonyl]-9-oxa-3,7-diazabicyclo[3.3.1]nonane-3-carboxylate (450.0 mg, 0.86 mmol, 1.0 eq), iron powder (239.81 mg, 4.29 mmol, 5.0 eq) and ammonium chloride (229.69 mg, 4.29 mmol, 5.0 eq) in ethanol (5 mL) and water (1 mL) was stirred at 50° C. for 3 h. The mixture was cooled to room temperature and filtered through a short plug of Celite (200 mg), rinsing the solid support with methanol (30 mL). The solvent was removed under reduced pressure to produce an oil which was dissolved with ethyl acetate (50 mL) and washed with water, dried (Na₂SO₄) and evaporated to dryness. The crude material was then purified by flash column chromatography eluting with 40% EtOAc in petroleum ether to give tert-butyl 7-[4-(4-amino-2-chlorobenzoyl)piperazine-1-carbonyl]-9-oxa-3,7-diazabicyclo[3.3.1]nonane-3-carboxylate (340.0 mg, 53.7% yield, 67% purity). MS [(M−Boc+H]⁺: 394.2.

### Step 3: 5-amino-1-[2,3-difluoro-4-(fluoromethoxy)phenyl]imidazole-4-carbonyl chloride

A solution of 5-amino-1-[2,3-difluoro-4-(fluoromethoxy)phenyl]imidazole-4-carboxylic acid (70.0 mg, 0.24 mmol, 1.0 eq) in thionyl chloride (289.98 mg, 2.44 mmol, 10.0 eq) was stirred at 25° C. for 1 h. The resulting solution was concentrated under reduced pressure to give 5-amino-1-[2,3-difluoro-4-(fluoromethoxy)phenyl]imidazole-4-carbonyl chloride (70.0 mg, 0.23 mmol, 71.8% yield). MS [(M−Cl+OCH₃)+H]⁺: 302.0

### Step 4: tert-butyl 7-[4-[4-[[5-amino-1-[2,3-difluoro-4-(fluoromethoxy)phenyl]imidazole-4-carbonyl]amino]-2-chlorobenzoyl]piperazine-1-carbonyl]-9-oxa-3,7-diazabicyclo[3.3.1]nonane-3-carboxylate

To a solution of tert-butyl 7-[4-(4-amino-2-chlorobenzoyl)piperazine-1-carbonyl]-9-oxa-3,7-diazabicyclo[3.3.1]nonane-3-carboxylate (113.14 mg, 0.23 mmol, 1.0 eq) and pyridine (0.19 mL, 2.29 mmol, 10.0 eq) in methylene chloride (2 mL) was added 5-amino-1-[2,3-difluoro-4-(fluoromethoxy)phenyl]imidazole-4-carbonyl chloride (70.0 mg, 0.23 mmol, 1.0 eq). The mixture was stirred at 25° C. for 3 h. The reaction was concentrated to dryness and the residue was taken up in EtOAc (5 mL). The organics were washed with 2×3 mL water followed by 3 ml brine. The organics were then separated and dried (Na₂SO₄) before concentrating to dryness. The crude was then purified by flash column chromatography eluting with 5% MeOH in methylene chloride to give tert-butyl 7-[4-[4-[[5-amino-1-[2,3-difluoro-4-(fluoromethoxy)phenyl]imidazole-4-carbonyl]amino]-2-chlorobenzoyl]piperazine-1-carbonyl]-9-oxa-3,7-diazabicyclo[3.3.1]nonane-3-carboxylate (120.0 mg, 0.16 mmol, 66.01% yield). MS [(M−Boc)+H]⁺: 663.1.

### Step 5: 5-amino-N-[3-chloro-4-[4-(9-oxa-3,7-diazabicyclo[3.3.1]nonane-3-carbonyl)piperazine-1-carbonyl]phenyl]-1-[2,3-difluoro-4-(fluoromethoxy)phenyl]imidazole-4-carboxamide; 1:1 2,2,2-trifluoroacetic acid

A solution of tert-butyl 7-[4-[4-[[5-amino-1-[2,3-difluoro-4-(fluoromethoxy)phenyl]imidazole-4-carbonyl]amino]-2-chlorobenzoyl]piperazine-1-carbonyl]-9-oxa-3,7-diazabicyclo[3.3.1]nonane-3-carboxylate (120.0 mg, 0.16 mmol, 1.0 eq) and trifluoroacetic acid (1.0 mL, 13 mmol, 82 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA)) to give 5-amino-N-[3-chloro-4-[4-(9-oxa-3,7-diazabicyclo[3.3.1]nonane-3-carbonyl)piperazine-1-carbonyl]phenyl]-1-[2,3-difluoro-4-(fluoromethoxy)phenyl]imidazole-4-carboxamide; 1:1 2,2,2-trifluoroacetic acid (80.0 mg, 0.1 mmol, 65.5% yield). MS [M+H]⁺: 663.2.

### Compound 134

### N-(4-(4-((1R,5S,6r)-3-azabicyclo[3.1.0]hexane-6-carbonyl)piperazine-1-carbonyl)-3-chlorophenyl)-5-amino-1-(1-(cyanomethyl)-3-(trifluoromethyl)-1H-pyrazol-4-yl)-1H-imidazole-4-carboxamide; 1:1 2,2,2-trifluoroacetic acid

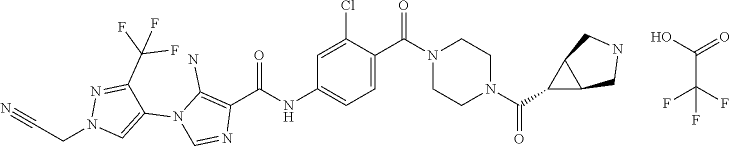

### Step 1: tert-butyl (1R,5S)-6-[4-[4-[[5-amino-1-[1-(cyanomethyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl (1R,5S)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (50.0 mg, 0.07 mmol, 1.0 eq) in DMF (1 mL) were added bromoacetonitrile (10.4 mg, 0.09 mmol, 1.2 eq) and potassium carbonate (29.95 mg, 0.22 mmol, 3.0 eq). The reaction was stirred at 25° C. for 3 h. The reaction was concentrated to dryness and the residue was taken up in EtOAc (20 mL). The organics were washed with 2×10 mL water followed by 10 mL brine. The organics were then separated and dried (Na₂SO₄) before concentration to dryness to give tert-butyl (1R,5S)-6-[4-[4-[[5-amino-1-[1-(cyanomethyl)-3-(trifluoromethyl)pyrazol-4-yl]imidazole-4-carbonyl]amino]-2-chloro-benzoyl]piperazine-1-carbonyl]-3-azabicyclo[3.1.0]hexane-3-carboxylate (40.0 mg, 75.73% yield). MS [(M−C₄H₈)+H]⁺: 675.0

### Step 2: 5-amino-N-[3-chloro-4-[4-[(1R,5S)-3-azabicyclo[3.1.0]hexane-6-carbonyl]piperazine-1-carbonyl]phenyl]-1-[1-(cyanomethyl)-3-(trifluoromethyl)pyrazol-4-yl]imidazole-4-carboxamide; 1:1 2,2,2-trifluoroacetic acid

A solution of tert-butyl (1R,5S)-6-[4-[4-[[5-amino-1-[1-(cyanomethyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (45.0 mg, 0.06 mmol, 1.0 eq) and trifluoroacetic acid (1.0 mL, 13 mmol, 210 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA)) to give 5-amino-N-[3-chloro-4-[4-[(1R,5S)-3-azabicyclo[3.1.0]hexane-6-carbonyl]piperazine-1-carbonyl]phenyl]-1-[1-(cyanomethyl)-3-(trifluoromethyl)pyrazol-4-yl]imidazole-4-carboxamide; 1:1 2,2,2-trifluoroacetic acid (17.0 mg, 0.02 mmol, 36.5% yield). MS [M+H]⁺: 631.3.

### Compound 135

### N-[4-[4-[(3aS,6aR)-2-methyl-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide

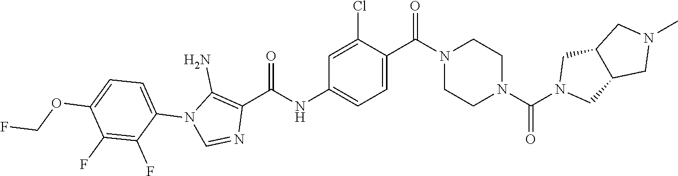 [SMILES: CN1C[C@@H]2CN(C(=O)N3CCN(C(=O)c4ccc(NC(=O)c5ncn(-c6ccc(OCF)c(F)c6F)c5N)cc4Cl)CC3)C[C@@H]2C1]

### Step 1: tert-butyl 4-(2-chloro-4-nitrobenzoyl)piperazine-1-carboxylate

To a solution of 2-chloro-4-nitrobenzoic acid (5.0 g, 24.81 mmol, 1.0 eq) in methylene chloride (50 mL) were added 1-BOC-piperazine (5.08 g, 27.29 mmol, 1.1 eq), propylphosphonic acid anhydride (31.57 g, 49.61 mmol, 2.0 eq) and N,N-diisopropylethylamine (9.62 g, 74.42 mmol, 3.0 eq). The resulting mixture was stirred at 25° C. for 1 h. The solvent was removed under reduced pressure. The residue was diluted with EtOAc (60 mL), washed with H₂O (3×60 mL), dried over Na₂SO₄ and concentrated under reduced pressure to give tert-butyl 4-(2-chloro-4-nitrobenzoyl)piperazine-1-carboxylate (7.7 g, 20.82 mmol, 83.9% yield). MS [M+Na]⁺: 392.0

### Step 2: (2-chloro-4-nitrophenyl)-piperazin-1-yl-methanone

A solution of tert-butyl 4-(2-chloro-4-nitrobenzoyl)piperazine-1-carboxylate (7.7 g, 20.82 mmol, 1.0 eq), hydrogen chloride/dioxane (30.0 mL) in methylene chloride (50 mL) was stirred at 25° C. for 1 h. The resulting solution was concentrated under reduced pressure to give (2-chloro-4-nitrophenyl)-piperazin-1-yl-methanone (4.9 g, 18.17 mmol, 87.3% yield). MS [M−H]⁻: 270.0

### Step 3: tert-butyl (3aS,6aR)-2-[4-(2-chloro-4-nitrobenzoyl)piperazine-1-carbonyl]-1,3,3a,4,6,6a-hexahydropyrrolo[3,4-c]pyrrole-5-carboxylate

A solution of (2-chloro-4-nitrophenyl)-piperazin-1-yl-methanone (300.0 mg, 1.11 mmol, 1.0 eq), triphosgene (165.06 mg, 0.56 mmol, 0.5 eq), triethylamine (0.47 mL, 3.34 mmol, 3.0 eq) and 1,1-dimethylethyl hexahydropyrrolo[3,4-c]pyrrole-2 (1H)-carbo (236.16 mg, 1.11 mmol, 1.0 eq) in methylene chloride (3 mL) was stirred at 25° C. for 1 h. The resulting solution was concentrated under reduced pressure. The residue was applied on a silica gel column and eluted with 1:1 petroleum ether:EtOAc to give tert-butyl (3aS,6aR)-2-[4-(2-chloro-4-nitrobenzoyl)piperazine-1-carbonyl]-1,3,3a,4,6,6a-hexahydropyrrolo[3,4-c]pyrrole-5-carboxylate (420.0 mg, 0.83 mmol, 74.3% yield). MS [M+Na]⁺: 530.1

### Step 4: tert-butyl (3aS,6aR)-2-[4-(4-amino-2-chlorobenzoyl)piperazine-1-carbonyl]-1,3,3a,4,6,6a-hexahydropyrrolo[3,4-c]pyrrole-5-carboxylate

A solution of tert-butyl (3aS,6aR)-2-[4-(2-chloro-4-nitrobenzoyl)piperazine-1-carbonyl]-1,3,3a,4,6,6a-hexahydropyrrolo[3,4-c]pyrrole-5-carboxylate (420.0 mg, 0.83 mmol, 1.0 eq), ammonium chloride (221.13 mg, 4.13 mmol, 5.0 eq) and iron (230.85 mg, 4.13 mmol, 5.0 eq) in ethanol (5 mL) and water (5 mL) was heated to 60° C. for 4 h. After this time the reaction was quenched by the addition of the saturated aqueous NaCl and then poured into separatory funnel and separated. The residue was extracted with ethyl acetate (30 mL). The organic layer was washed with water, dried (Na₂SO₄) and evaporated to dryness to afford tert-butyl (3aS,6aR)-2-[4-(4-amino-2-chlorobenzoyl)piperazine-1-carbonyl]-1,3,3a,4,6,6a-hexahydropyrrolo[3,4-c]pyrrole-5-carboxylate (320.0 mg, 0.67 mmol, 81% yield). MS [M−Boc+H]⁺: 378.1

### Step 5: 5-amino-1-[2,3-difluoro-4-(fluoromethoxy)phenyl]imidazole-4-carbonyl chloride

A solution of 5-amino-1-[2,3-difluoro-4-(fluoromethoxy)phenyl]imidazole-4-carboxylic acid (60.0 mg, 0.21 mmol, 1.0 eq) in thionyl chloride (2.0 mL, 28 mmol, 130 eq) was stirred at 25° C. for 1 h. The resulting solution was concentrated under reduced pressure to give 5-amino-1-[2,3-difluoro-4-(fluoromethoxy)phenyl]imidazole-4-carbonyl chloride (60.0 mg, 0.2 mmol, 94% yield). MS [M−Cl+OMe+H]⁺: 302.1

### Step 6: tert-butyl (3aR,6aS)-2-[4-[4-[[5-amino-1-[2,3-difluoro-4-(fluoromethoxy)phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate

A solution of tert-butyl (3aR,6aS)-2-[4-(4-amino-2-chlorobenzoyl)piperazine-1-carbonyl]-1,3,3a,4,6,6a-hexahydropyrrolo[3,4-c]pyrrole-5-carboxylate (65.68 mg, 0.14 mmol, 0.7 eq) and pyridine (0.32 mL, 3.93 mmol, 20.0 eq) in methylene chloride (2 mL) was stirred at 25° C. 5-Amino-1-[2,3-difluoro-4-(fluoromethoxy)phenyl]imidazole-4-carbonyl chloride (60.0 mg, 0.2 mmol, 1.0 eq) in methylene chloride (2 mL) was added, and the temperature increased to 25° C. for 1 h. The resulting solution was concentrated under reduced pressure. The residue was applied on a silica gel column and eluted with 10:1 methylene chloride:MeOH to give tert-butyl (3aR,6aS)-2-[4-[4-[[5-amino-1-[2,3-difluoro-4-(fluoromethoxy)phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (110.0 mg, 0.15 mmol, 75.0% yield). MS [M−Boc+H]⁺: 647.0

### Step 7: N-[4-[4-[(3aS,6aR)-2,3,3a,4,6,6a-hexahydro-1H-pyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide

To a solution of tert-butyl (3aR,6aS)-2-[4-[4-[[5-amino-1-[2,3-difluoro-4-(fluoromethoxy)phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (110.0 mg, 0.15 mmol, 1.0 eq) in methylene chloride (2 mL) was added hydrogen chloride (2.0 mL, 1M in dioxane). The solution was stirred at 25° C. for 1 h and then concentrated under reduced pressure to give N-[4-[4-[(3aS,6aR)-2,3,3a,4,6,6a-hexahydro-1H-pyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (75.0 mg, 0.12 mmol, 78.7% yield). MS [M+H]⁺: 647.1

### Step 8: N-[4-[4-[(3aS,6aR)-2-methyl-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide

A solution of N-[4-[4-[(3aS,6aR)-2,3,3a,4,6,6a-hexahydro-1H-pyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (65.0 mg, 0.1 mmol, 1.0 eq), polyformaldehyde (9.05 mg, 0.3 mmol, 3.0 eq), sodium acetate (24.72 mg, 0.3 mmol, 3.0 eq), sodium cyanoborohydride (31.56 mg, 0.5 mmol, 5.0 eq) in methanol (3 mL) was stirred at 25° C. for 12 h. The resulting solution was concentrated under reduced pressure. The crude product was purified by Prep-HPLC (Gemini C18 150×21.2 mm, 5 μm, MeCN-H₂O (0.1% TFA), 20-40) to afford N-[4-[4-[(3aS,6aR)-2-methyl-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (38.4 mg, 0.06 mmol, 57.6% yield). MS [M+H]⁺: 661.2

### Compound 136

### N-[4-[4-[(3aS,6aR)-2-methyl-1,3,3a,4,6,6a-hexahydropyrrolo[3,4-c]pyrrole-5-carbonyl]piperazine-1-carbonyl]-3-fluorophenyl]-5-amino-1-[2,3-difluoro-4-(fluoromethoxy)phenyl]imidazole-4-carboxamide; 1:1 formic acid

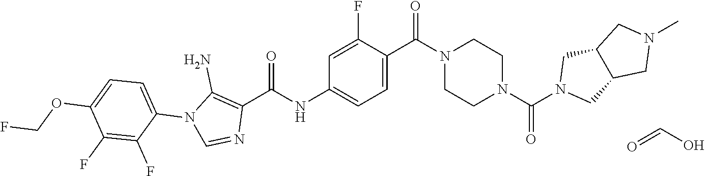 [SMILES: CN1C[C@@H]2CN(C(=O)N3CCN(C(=O)c4ccc(NC(=O)c5ncn(-c6ccc(OCF)c(F)c6F)c5N)cc4F)CC3)C[C@@H]2C1 ; O=CO]

### Step 1: tert-butyl (3aR,6aS)-2-[4-[4-[[5-amino-1-[2,3-difluoro-4-(fluoromethoxy)phenyl]imidazole-4-carbonyl]amino]-2-fluorobenzoyl]piperazine-1-carbonyl]-1,3,3a,4,6,6a-hexahydropyrrolo[3,4-c]pyrrole-5-carboxylate

A solution of 5-amino-1-[2,3-difluoro-4-(fluoromethoxy)phenyl]imidazole-4-carbonyl chloride (50.0 mg, 0.16 mmol, 1.0 eq), tert-butyl (3aR,6aS)-2-[4-(4-amino-2-fluorobenzoyl)piperazine-1-carbonyl]-1,3,3a,4,6,6a-hexahydropyrrolo[3,4-c]pyrrole-5-carboxylate (52.85 mg, 0.11 mmol, 0.7 eq) and pyridine (0.26 mL, 3.27 mmol, 20.0 eq) in methylene chloride (1 mL) was stirred at 20° C. for 1 h. After concentration under reduced pressure, the residue was purified by flash column chromatography on silica gel to give tert-butyl (3aR,6aS)-2-[4-[4-[[5-amino-1-[2,3-difluoro-4-(fluoromethoxy)phenyl]imidazole-4-carbonyl]amino]-2-fluorobenzoyl]piperazine-1-carbonyl]-1,3,3a,4,6,6a-hexahydro-pyrrolo[3,4-c]pyrrole-5-carboxylate (100.0 mg, 0.14 mmol, 83.7% yield). MS [(M−Boc)+H]⁺: 631.3

### Step 2: N-[4-[4-[(3aS,6aR)-2,3,3a,4,6,6a-hexahydro-1H-pyrrolo[3,4-c]pyrrole-5-carbonyl]piperazine-1-carbonyl]-3-fluoro-phenyl]-5-amino-1-[2,3-difluoro-4-(fluoromethoxy)phenyl]imidazole-4-carboxamide

A solution of tert-butyl (3aR,6aS)-2-[4-[4-[[5-amino-1-[2,3-difluoro-4-(fluoromethoxy)phenyl]imidazole-4-carbonyl]amino]-2-fluorobenzoyl]piperazine-1-carbonyl]-1,3,3a,4,6,6a-hexahydro-pyrrolo[3,4-c]pyrrole-5-carboxylate (100.0 mg, 0.14 mmol, 1.0 eq) and trifluoroacetic acid (1.0 mL, 12.98 mmol, 94.84 eq) in methylene chloride (4 mL) was stirred at 25° C. for 1 h. The resulting solution was concentrated under reduced pressure to give N-[4-[4-[(3aS,6aR)-2,3,3a,4,6,6a-hexahydro-1H-pyrrolo[3,4-c]pyrrole-5-carbonyl]piperazine-1-carbonyl]-3-fluorophenyl]-5-amino-1-[2,3-difluoro-4-(fluoromethoxy)phenyl]imidazole-4-carboxamide (75.0 mg, 0.12 mmol, 82.6% yield) as the crude product. MS [M+H]⁺: 631.3.

### Step 3: N-[4-[4-[(3aS,6aR)-2-methyl-1,3,3a,4,6,6a-hexahydropyrrolo[3,4-c]pyrrole-5-carbonyl]piperazine-1-carbonyl]-3-fluorophenyl]-5-amino-1-[2,3-difluoro-4-(fluoromethoxy)phenyl]imidazole-4-carboxamide; 1:1 formic acid

A solution of N-[4-[4-[(3aS,6aR)-2,3,3a,4,6,6a-hexahydro-1H-pyrrolo[3,4-c]pyrrole-5-carbonyl]piperazine-1-carbonyl]-3-fluorophenyl]-5-amino-1-[2,3-difluoro-4-(fluoromethoxy)phenyl]imidazole-4-carboxamide (70.0 mg, 0.11 mmol, 1.0 eq), paraformadehyde (61.68 mg, 0.11 mmol, 1.0 eq), NaCNBH₃ (34.97 mg, 0.56 mmol, 5.0 eq) and NaOAc (27.31 mg, 0.33 mmol, 3.0 eq) in methanol (2 mL) was stirred at 25° C. for 2 h. The resulting solution was extraction by water and ethyl acetate and then concentrated under reduced pressure at 30° C. The resulting solution was concentrated under reduced pressure. The crude product was purified by Prep-HPLC (Gemini C18 150×21.2 mm, 5 μm, MeCN-H₂O (0.1% TFA), 15-40) to afford N-[4-[4-[(3aS,6aR)-2-methyl-1,3,3a,4,6,6a-hexahydropyrrolo[3,4-c]pyrrole-5-carbonyl]piperazine-1-carbonyl]-3-fluorophenyl]-5-amino-1-[2,3-difluoro-4-(fluoromethoxy)phenyl]imidazole-4-carboxamide; 1:1 formic acid (13.5 mg, 0.02 mmol, 18.3% yield). MS [M+H]⁺: 648.3

### Compound 137

### 5-amino-N-(3-chloro-4-(4-(2-methyl-5-oxa-2,8-diazaspiro[3.5]nonane-8-carbonyl)piperazine-1-carbonyl)phenyl)-1-(2,3-difluoro-4-(fluoromethoxy)phenyl)-1H-imidazole-4-carboxamide; 1:1 2,2,2-trifluoroacetate

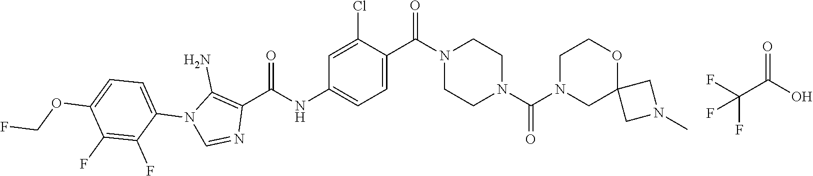

### Step 1: tert-butyl 8-(4-(2-chloro-4-nitrobenzoyl)piperazine-1-carbonyl)-5-oxa-2,8-diazaspiro[3.5]nonane-2-carboxylate

To a solution of (2-chloro-4-nitro-phenyl)-piperazin-1-yl-methanone (100.0 mg, 0.37 mmol, 1.0 eq) in methylene chloride (3 mL) was added triphosgene (44.01 mg, 0.15 mmol, 0.4 eq). The reaction was stirred at 25° C. for 0.5 h, and then triethylamine (0.78 mL, 5.56 mmol, 15.0 eq) and tert-butyl 5-oxa-2,8-diazaspiro[3.5]nonane-2-carboxylate (84.65 mg, 0.37 mmol, 1.0 eq) were added. The reaction was stirred at 25° C. for 3 h and then concentrated to dryness. The residue was taken up in methylene chloride (10 mL), and the organics were washed with 2×10 ml water and 10 mL brine. The organics were then separated and dried (MgSO₄) before concentrating to dryness. The crude was then purified by flash column chromatography eluting with 80% EtOAc in petroleum ether to afford tert-butyl 8-[4-(2-chloro-4-nitrobenzoyl)piperazine-1-carbonyl]-5-oxa-2,8-diazaspiro[3.5]nonane-2-carboxylate (100.0 mg, 0.19 mmol, 39.4% yield). MS [M+H−Boc]⁺: 424.1

### Step 2: tert-butyl 8-[4-(4-amino-2-chlorobenzoyl)piperazine-1-carbonyl]-5-oxa-2,8-diazaspiro[3.5]nonane-2-carboxylate

To a solution of tert-butyl 8-[4-(2-chloro-4-nitrobenzoyl)piperazine-1-carbonyl]-5-oxa-2,8-diazaspiro[3.5]nonane-2-carboxylate (100.0 mg, 0.19 mmol, 1.0 eq) in ethanol (5 mL) and water (0.5 mL) were added iron powder (106.59 mg, 1.91 mmol, 10.0 eq) and ammonium chloride (102.09 mg, 1.91 mmol, 10.0 eq). The reaction was stirred at 50° C. for 4 h. The reaction was concentrated to dryness. The residue was taken up in EtOAc (30 mL) and the organics washed with 2×20 mL water followed 20 mL brine. The organics were then separated and dried (MgSO₄) before concentrating to dryness to afford tert-butyl 8-[4-(4-amino-2-chlorobenzoyl) piperazine-1-carbonyl]-5-oxa-2,8-diazaspiro[3.5]nonane-2-carboxylate (75.0 mg, 0.15 mmol, 79.6% yield). MS [M+H−Boc]⁺: 394.1

### Step 3: 5-amino-1-[2,3-difluoro-4-(fluoromethoxy)phenyl]imidazole-4-carbonyl chloride

A mixture of 5-amino-1-[2,3-difluoro-4-(fluoromethoxy)phenyl]imidazole-4-carboxylic acid (10.0 mg, 0.03 mmol, 1.0 eq) and thionyl chloride (2 mL) was stirred at 20° C. for 1 hour under N₂. The resulting solution was evaporated to dryness to afford the title compound 5-amino-1-[2,3-difluoro-4-(fluoromethoxy)phenyl]imidazole-4-carbonyl chloride (10.0 mg, 0.03 mmol, 83.6% yield), which was used without further purification. MS: 302.0 [M−Cl+OCH₃+H]⁺

### Step 4: tert-butyl 8-[4-[4-[[5-amino-1-[2,3-difluoro-4-(fluoromethoxy)phenyl]imidazole-4-carbonyl]amino]-2-chlorobenzoyl]piperazine-1-carbonyl]-5-oxa-2,8-diazaspiro[3.5]nonane-2-carboxylate

To a solution of tert-butyl 8-[4-(4-amino-2-chlorobenzoyl)piperazine-1-carbonyl]-5-oxa-2,8-diazaspiro[3.5]nonane-2-carboxylate (67.88 mg, 0.14 mmol, 0.7 eq) in methylene chloride (3 mL) were added pyridine (0.32 mL, 3.93 mmol, 20.0 eq) and 5-amino-1-[2,3-difluoro-4-(fluoromethoxy)phenyl]imidazole-4-carbonyl chloride (60.0 mg, 0.2 mmol, 1.0 eq). The reaction was stirred at 25° C. for 6 h and then concentrated to dryness. The residue was taken up in methylene chloride (20 mL). The organics washed with 2×20 ml water followed by 20 mL brine. The organics were then separated and dried (MgSO₄) before concentrating to dryness. The crude was then purified by flash column chromatography eluting with 8% MeOH in methylene chloride to afford tert-butyl 8-[4-[4-[[5-amino-1-[2,3-difluoro-4-(fluoromethoxy)phenyl]imidazole-4-carbonyl]amino]-2-chlorobenzoyl]piperazine-1-carbonyl]-5-oxa-2,8-diazaspiro[3.5]nonane-2-carboxylate (100.0 mg, 0.13 mmol, 66.8% yield). MS [M+H−Boc]⁺: 663.0

### Step 5: 5-amino-N-[3-chloro-4-[4-(5-oxa-2,8-diazaspiro[3.5]nonane-8-carbonyl)piperazine-1-carbonyl]phenyl]-1-[2,3-difluoro-4-(fluoromethoxy)phenyl]imidazole-4-carboxamide; 1:1 2,2,2-trifluoroacetic acid

To a solution of tert-butyl 8-[4-[4-[[5-amino-1-[2,3-difluoro-4-(fluoromethoxy)phenyl]imidazole-4-carbonyl]amino]-2-chlorobenzoyl]piperazine-1-carbonyl]-5-oxa-2,8-diazaspiro[3.5]nonane-2-carboxylate (100.0 mg, 0.13 mmol, 1.0 eq) in methylene chloride (3 mL) was added trifluoroacetic acid (1.0 mL). The reaction was stirred at 25° C. for 1 h. The resulting solution was concentrated under reduced pressure, and the crude product was purified by Prep-HPLC (Gemini C18 150×21.2 mm, 5 μm, MeCN-H₂O (0.1% TFA), 5-50) to afford 5-amino-N-[3-chloro-4-[4-(5-oxa-2,8-diazaspiro[3.5]nonane-8-carbonyl)piperazine-1-carbonyl]phenyl]-1-[2,3-difluoro-4-(fluoromethoxy)phenyl]imidazole-4-carboxamide; 1:1 2,2,2-trifluoroacetic acid (80.0 mg, 0.1 mmol, 77.2% yield). MS [M+H]⁺: 663.1

### Step 6: 5-amino-N-[3-chloro-4-[4-(2-methyl-5-oxa-2,8-diazaspiro[3.5]nonane-8-carbonyl)piperazine-1-carbonyl]phenyl]-1-[2,3-difluoro-4-(fluoromethoxy)phenyl]imidazole-4-carboxamide; 1:1 2,2,2-trifluoroacetic acid

A solution of 5-amino-N-[3-chloro-4-[4-(5-oxa-2,8-diazaspiro[3.5]nonane-8-carbonyl)piperazine-1-carbonyl]phenyl]-1-[2,3-difluoro-4-(fluoromethoxy)phenyl]imidazole-4-carboxamide (35.0 mg, 0.05 mmol, 1.0 eq), polyformaldehyde (4.76 mg, 0.16 mmol, 3.0 eq), sodium acetate (12.99 mg, 0.16 mmol, 3.0 eq), sodium cyanoborohydride (16.59 mg, 0.26 mmol, 5.0 eq) in methanol (3 mL) was stirred at 25° C. for 10 h. The resulting solution was concentrated under reduced pressure, and the crude product was purified by Prep-HPLC (Gemini C18 150×21.2 mm, 5 μm, MeCN-H₂O (0.1% TFA), 20-40) to afford 5-amino-N-[3-chloro-4-[4-(2-methyl-5-oxa-2,8-diazaspiro[3.5]nonane-8-carbonyl)piperazine-1-carbonyl]phenyl]-1-[2,3-difluoro-4-(fluoromethoxy)phenyl]imidazole-4-carboxamide; 1:1 2,2,2-trifluoroacetic acid (13.3 mg, 0.02 mmol, 36.5% yield). MS [M+H]⁺: 677.2

### Compound 138

### 5-amino-N-[3-chloro-4-[4-[6-(3-hydroxypropyl)-2,6-diazaspiro[3.3]heptane-2-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

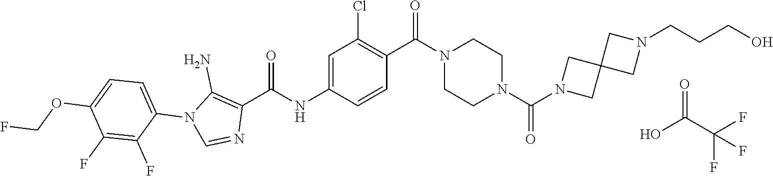

### Step 1: 5-amino-N-[4-[4-[6-[3-[tert-butyl(dimethyl)silyl]oxypropyl]-2,6-diazaspiro[3.3]heptane-2-carbonyl]piperazine-1-carbonyl]-3-chlorophenyl]-1-[2,3-difluoro-4-(fluoromethoxy)phenyl]imidazole-4-carboxamide

A solution of 5-amino-N-[3-chloro-4-[4-(2,6-diazaspiro[3.3]heptane-2-carbonyl)piperazine-1-carbonyl]phenyl]-1-[2,3-difluoro-4-(fluoromethoxy)phenyl]imidazole-4-carboxamide;2,2,2-trifluoroacetic acid (60.0 mg, 0.08 mmol, 1.0 eq), 3-[tert-butyl(dimethyl)silyl]oxypropanal (45.38 mg, 0.24 mmol, 3.0 eq), sodium acetate (19.77 mg, 0.24 mmol, 3.0 eq), and sodium cyanoborohydride (25.24 mg, 0.4 mmol, 5.0 eq) in methanol (2 mL) was stirred at 25° C. for 1 h. The resulting solution was concentrated under reduced pressure. The residue was applied on a silica gel column and eluted with 10:1 methylene chloride/MeOH to give 5-amino-N-[4-[4-[6-[3-[tert-butyl(dimethyl)silyl]oxypropyl]-2,6-diazaspiro[3.3]heptane-2-carbonyl]piperazine-1-carbonyl]-3-chlorophenyl]-1-[2,3-difluoro-4-(fluoromethoxy)phenyl]imidazole-4-carboxamide (50.0 mg, 0.06 mmol, 71.9% yield). MS [M+H]⁺: 805.2.

### Step 2: 5-amino-N-[3-chloro-4-[4-[6-(3-hydroxypropyl)-2,6-diazaspiro[3.3]heptane-2-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

A solution of 5-amino-N-[4-[4-[6-[3-[tert-butyl(dimethyl)silyl]oxypropyl]-2,6-diazaspiro[3.3]heptane-2-carbonyl]piperazine-1-carbonyl]-3-chlorophenyl]-1-[2,3-difluoro-4-(fluoromethoxy)phenyl]imidazole-4-carboxamide (40.0 mg, 0.05 mmol, 1.0 eq) and triethylamine trihydrofluoride (80.07 mg, 0.5 mmol, 10.0 eq) in THF (2 mL) was stirred at 25° C. for 1 h. The resulting solution was concentrated under reduced pressure. The residue was purified by Prep-HPLC (Gemini-C18 150×21.2 mm, 5 μm, MeCN-H₂O (0.1% TFA), 25-40) to give 5-amino-N-[3-chloro-4-[4-[6-(3-hydroxypropyl)-2,6-diazaspiro[3.3]heptane-2-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (14.9 mg, 0.02 mmol, 36.9% yield). MS [M+H]⁺: 691.2.

### Compound 139

### 5-amino-N-(3-chloro-4-(4-(6-(2-(2-hydroxyethoxy)ethyl)-2,6-diazaspiro[3.3]heptane-2-carbonyl)piperazine-1-carbonyl)phenyl)-1-(2,3-difluoro-4-(fluoromethoxy)phenyl)-1H-imidazole-4-carboxamide; 1:1 2,2,2-trifluoroacetic acid

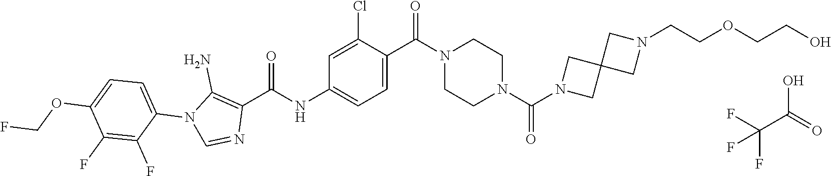 [SMILES: Nc1c(C(=O)Nc2ccc(C(=O)N3CCN(C(=O)N4CC5(CN(CCOCCO)C5)C4)CC3)c(Cl)c2)ncn1-c1ccc(OCF)c(F)c1F ; O=C(O)C(F)(F)F]

### Step 1: 5-amino-N-[4-[4-[6-[2-[2-[tert-butyl(diphenyl)silyl]oxyethoxy]ethyl]-2,6-diazaspiro[3.3]heptane-2-carbonyl]piperazine-1-carbonyl]-3-chlorophenyl]-1-[2,3-difluoro-4-(fluoromethoxy)phenyl]imidazole-4-carboxamide

A solution of 5-amino-N-[3-chloro-4-[4-(2,6-diazaspiro[3.3]heptane-2-carbonyl)piperazine-1-carbonyl]phenyl]-1-[2,3-difluoro-4-(fluoromethoxy)phenyl]imidazole-4-carboxamide;2,2,2-trifluoroacetic acid (50.0 mg, 0.07 mmol, 1.0 eq), 2-[2-[tert-butyl(diphenyl)silyl]oxyethoxy]acetaldehyde (45.85 mg, 0.13 mmol, 2.0 eq), sodium acetate (16.47 mg, 0.2 mmol, 3.0 eq), sodium cyanoborohydride (21.03 mg, 0.33 mmol, 5.0 eq) in methanol (2 mL) was stirred at 25° C. for 1 h. The resulting solution was concentrated under reduced pressure. The residue was applied on a silica gel column and eluted with 10:1 methylene chloride:MeOH to give 5-amino-N-[4-[4-[6-[2-[2-[tert-butyl(diphenyl)silyl]oxyethoxy]ethyl]-2,6-diazaspiro[3.3]heptane-2-carbonyl]piperazine-1-carbonyl]-3-chlorophenyl]-1-[2,3-difluoro-4-(fluoromethoxy)phenyl]imidazole-4-carboxamide (50.0 mg, 0.05 mmol, 77.8% yield). MS [M+H]⁺: 959.2.

### Step 2: 5-amino-N-[3-chloro-4-[4-[6-[2-(2-hydroxyethoxy)ethyl]-2,6-diazaspiro[3.3]heptane-2-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

A solution of 5-amino-N-[4-[4-[6-[2-[2-[tert-butyl(diphenyl)silyl]oxyethoxy]ethyl]-2,6-diazaspiro[3.3]heptane-2-carbonyl]piperazine-1-carbonyl]-3-chlorophenyl]-1-[2,3-difluoro-4-(fluoromethoxy)phenyl]imidazole-4-carboxamide (47.66 mg, 0.05 mmol, 1.0 eq) and triethylamine trihydrofluoride (80.07 mg, 0.5 mmol, 10.0 eq) in THF (2 mL) was stirred at 25° C. for 1 h. The resulting solution was concentrated under reduced pressure. The residue was purified by Prep-HPLC (Gemini-C18 150×21.2 mm, 5 μm, MeCN-H₂O (0.1% TFA), 25-40) to give 5-amino-N-[3-chloro-4-[4-[6-[2-(2-hydroxyethoxy)ethyl]-2,6-diazaspiro[3.3]heptane-2-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (13.6 mg, 0.02 mmol, 37.8% yield). MS [M+H]⁺: 721.2.

### Compound 140

### 5-amino-N-[3-chloro-4-[4-[6-(2-hydroxyethyl)-2,6-diazaspiro[3.3]heptane-2-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

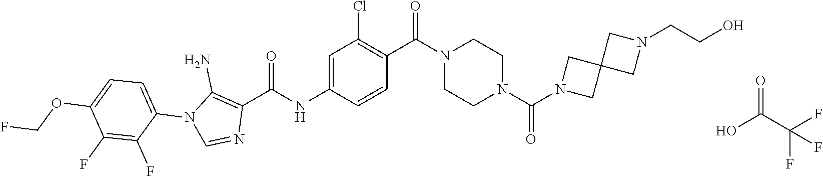 [SMILES: Nc1c(C(=O)Nc2ccc(C(=O)N3CCN(C(=O)N4CC5(CN(CCO)C5)C4)CC3)c(Cl)c2)ncn1-c1ccc(OCF)c(F)c1F ; O=C(O)C(F)(F)F]

### Step 1: tert-butyl 6-[4-(2-chloro-4-nitrobenzoyl)piperazine-1-carbonyl]-2,6-diazaspiro[3.3]heptane-2-carboxylate

To a solution of (2-chloro-4-nitrophenyl)-piperazin-1-yl-methanone hydrochloride (300.0 mg, 0.98 mmol, 1.0 eq) in methylene chloride (2 mL) was added triphosgene (174.62 mg, 0.59 mmol, 0.6 eq) at 25° C. After stirring for 0.5 h, triethylamine (989.71 mg, 9.8 mmol, 10.0 eq) and tert-butyl 2,6-diazaspiro[3.3]heptane-2-carboxylate;oxalic acid (572.14 mg, 1.18 mmol, 1.2 eq) were added, and the resulting mixture was stirred at 25° C. for 1 h. The resulting solution was concentrated under reduced pressure. The residue was applied on a silica gel column and eluted with 1:20 MeOH:methylene-chloride to give tert-butyl 6-[4-(2-chloro-4-nitrobenzoyl)piperazine-1-carbonyl]-2,6-diazaspiro[3.3]heptane-2-carboxylate (250.0 mg, 0.51 mmol, 51.6% yield). MS [(M−t−Bu+H]⁺: 438.1.

### Step 2: tert-butyl 6-[4-(4-amino-2-chlorobenzoyl)piperazine-1-carbonyl]-2,6-diazaspiro[3.3]heptane-2-carboxylate

A solution of tert-butyl 6-[4-(2-chloro-4-nitrobenzoyl)piperazine-1-carbonyl]-2,6-diazaspiro[3.3]heptane-2-carboxylate (240.0 mg, 0.49 mmol, 1.0 eq), iron powder (130.05 mg, 2.43 mmol, 5.0 eq) and ammonium chloride (131.19 mg, 2.43 mmol, 5.0 eq) in ethanol (5 mL) and water (2 mL) was stirred at 50° C. for 3 h. The solid was filtered off. The crude was then purified by flash column chromatography eluting with 40% EtOAc in petroleum ether to give tert-butyl 6-[4-(4-amino-2-chlorobenzoyl)piperazine-1-carbonyl]-2,6-diazaspiro[3.3]heptane-2-carboxylate (170.0 mg, 0.37 mmol, 75.4% yield). MS [(M+H]⁺: 464.1.

### Step 3: 5-amino-1-[2,3-difluoro-4-(fluoromethoxy)phenyl]imidazole-4-carbonyl chloride

A solution of 5-amino-1-[2,3-difluoro-4-(fluoromethoxy)phenyl]imidazole-4-carboxylic acid (110.0 mg, 0.17 mmol, 1.0 eq) in thionyl chloride (1 mL) was stirred at 25° C. for 1 h. The resulting solution was concentrated under reduced pressure to give 5-amino-1-[2,3-difluoro-4-(fluoromethoxy)phenyl]imidazole-4-carbonyl chloride (100.0 mg, 0.16 mmol, 53.6% yield). MS [(M−Cl+OCH₃)+H]⁺: 302.1.

### Step 4: tert-butyl 6-[4-[4-[[5-amino-1-[2,3-difluoro-4-(fluoromethoxy)phenyl]imidazole-4-carbonyl]amino]-2-chlorobenzoyl]piperazine-1-carbonyl]-2,6-diazaspiro[3.3]heptane-2-carboxylate

To a solution of tert-butyl 6-[4-(4-amino-2-chlorobenzoyl)piperazine-1-carbonyl]-2,6-diazaspiro[3.3]heptane-2-carboxylate (170.0 mg, 0.37 mmol, 1.0 eq) and pyridine (0.03 mL, 0.37 mmol, 1.0 eq) in methylene chloride (3 mL) was added 5-amino-1-[2,3-difluoro-4-(fluoromethoxy)phenyl]imidazole-4-carbonyl chloride (100.0 mg, 0.13 mmol, 1.0 eq) at 25° C., and the mixture was stirred for 1 h. The resulting solution was concentrated under reduced pressure. The residue was applied on a silica gel column and eluted with 1:20 MeOH:methylene chloride to give tert-butyl 6-[4-[4-[[5-amino-1-[2,3-difluoro-4-(fluoromethoxy)phenyl]imidazole-4-carbonyl]amino]-2-chlorobenzoyl]piperazine-1-carbonyl]-2,6-diazaspiro[3.3]heptane-2-carboxylate (230.0 mg, 0.25 mmol, 67.0% yield). MS [(M−Boc)+H]⁺: 633.1.

### Step 5: 5-amino-N-[3-chloro-4-[4-(2,6-diazaspiro[3.3]heptane-2-carbonyl)piperazine-1-carbonyl]phenyl]-1-[2,3-difluoro-4-(fluoromethoxy)phenyl]imidazole-4-carboxamide

A solution of tert-butyl 6-[4-[4-[[5-amino-1-[2,3-difluoro-4-(fluoromethoxy)phenyl]imidazole-4-carbonyl]amino]-2-chlorobenzoyl]piperazine-1-carbonyl]-2,6-diazaspiro[3.3]heptane-2-carboxylate (230.0 mg, 0.31 mmol, 1.0 eq) in methylene chloride (2 mL) was stirred at 25° C. for 1 h. The resulting solution was concentrated under reduced pressure to afford 5-amino-N-[3-chloro-4-[4-(2,6-diazaspiro[3.3]heptane-2-carbonyl)piperazine-1-carbonyl]phenyl]-1-[2,3-difluoro-4-(fluoromethoxy)phenyl]imidazole-4-carboxamide (210.0 mg, 0.33 mmol, 97.3% yield). MS [M+H]⁺: 633.2.

### Step 6: 5-amino-N-[4-[4-[6-[2-[tert-butyl(dimethyl)silyl]oxyethyl]-2,6-diazaspiro[3.3]heptane-2-carbonyl]piperazine-1-carbonyl]-3-chlorophenyl]-1-[2,3-difluoro-4-(fluoromethoxy)phenyl]imidazole-4-carboxamide

A solution of 5-amino-N-[3-chloro-4-[4-(2,6-diazaspiro[3.3]heptane-2-carbonyl)piperazine-1-carbonyl]phenyl]-1-[2,3-difluoro-4-(fluoromethoxy)phenyl]imidazole-4-carboxamide;2,2,2-trifluoroacetic acid (60.0 mg, 0.08 mmol, 1.0 eq), (tert-butyldimethylsilyloxy) acetaldehyde (42.0 mg, 0.24 mmol, 3.0 eq), sodium acetate (19.77 mg, 0.24 mmol, 3.0 eq), sodium cyanoborohydride (25.24 mg, 0.4 mmol, 5.0 eq) in methanol (2 mL) was stirred at 25° C. for 1 h. The resulting solution was concentrated under reduced pressure. The residue was applied on a silica gel column and eluted with 10:1 methylene chloride:MeOH to give 5-amino-N-[4-[4-[6-[2-[tert-butyl(dimethyl)silyl]oxyethyl]-2,6-diazaspiro[3.3]heptane-2-carbonyl]piperazine-1-carbonyl]-3-chlorophenyl]-1-[2,3-difluoro-4-(fluoromethoxy)phenyl]imidazole-4-carboxamide (50.0 mg, 0.06 mmol, 74% yield). MS [M+H]⁺: 791.3.

### Step 7: 5-amino-N-[3-chloro-4-[4-[6-(2-hydroxyethyl)-2,6-diazaspiro[3.3]heptane-2-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

A solution of 5-amino-N-[4-[4-[6-[2-[tert-butyl(dimethyl)silyl]oxyethyl]-2,6-diazaspiro[3.3]heptane-2-carbonyl]piperazine-1-carbonyl]-3-chlorophenyl]-1-[2,3-difluoro-4-(fluoromethoxy)phenyl]imidazole-4-carboxamide (40.0 mg, 0.05 mmol, 1.0 eq) and triethylamine trihydrofluoride (81.49 mg, 0.51 mmol, 10.0 eq) in THF (1 mL) was stirred at 25° C. for 1 h. The resulting solution was concentrated under reduced pressure. The residue was purified by Prep-HPLC (Gemini-C18 150×21.2 mm, 5 μm, MeCN— H₂O (0.1% TFA), 20-50) to give 5-amino-N-[3-chloro-4-[4-[6-(2-hydroxyethyl)-2,6-diazaspiro[3.3]heptane-2-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (11.0 mg, 0.01 mmol, 31.8% yield). MS [M+H]⁺: 677.3.

### Compound 141

### N-[4-[4-[(3aS,6aR)-2-[2-(2-hydroxyethoxy)ethyl]-1,3,3a, 4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide

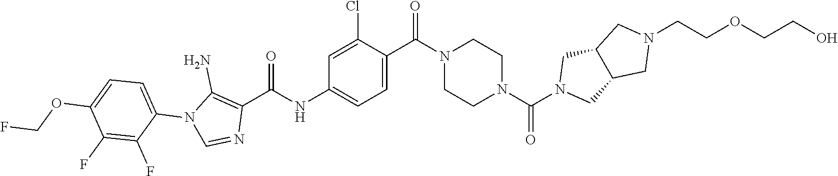 [SMILES: Nc1c(C(=O)Nc2ccc(C(=O)N3CCN(C(=O)N4C[C@H]5CN(CCOCCO)C[C@H]5C4)CC3)c(Cl)c2)ncn1-c1ccc(OCF)c(F)c1F]

### Step 1: 2-[2-[tert-butyl(diphenyl)silyl]oxyethoxy]ethanol

To a solution of diethylene glycol (2.0 g, 18.85 mmol, 1.0 eq) in pyridine (1.49 g, 18.85 mmol, 1.0 eq) was added tert-butylchlorodiphenylsilane (1.55 g, 5.65 mmol, 0.3 eq) at 0° C. The reaction mixture was stirred at 25° C. for 15 h, then poured into water (20 mL) and extracted with EtOAc (3×20 mL). The combined organic layers were concentrated and purified via silica gel chromatography (3:2 petroleum ether:EtOAc) to give 2-[2-[tert-butyl(diphenyl)silyl]oxyethoxy]ethanol (1.7 g, 4.93 mmol, 26.2% yield). MS [M+Na]⁺: 367.1

### Step 2: 2-[2-[tert-butyl(diphenyl)silyl]oxyethoxy]acetaldehyde

To a solution of oxalyl chloride (324.21 mg, 2.55 mmol, 1.1 eq) in methylene chloride (2 mL) was added dimethyl sulfoxide (399.13 mg, 5.11 mmol, 2.2 eq) at −78° C. The resulting mixture was stirred for 0.5 h. Next, 2-[2-[tert-butyl(diphenyl)silyl]oxyethoxy]ethanol (800.0 mg, 2.32 mmol, 1.0 eq) in methylene chloride (2 mL) was added, and the reaction was stirred at −78° C. for 1 h. Then triethylamine (1.62 mL, 11.61 mmol, 5.0 eq) was added to the solution. The temperature was increased to room temperature. The resulting solution was diluted with 10 ml of water, then extracted with 3×10 mL of EtOAc. The organic layers were combined, washed with brine, dried and concentrated under reduced pressure. The residue was applied on a silica gel column and eluted with 3:1 petroleum ether:EtOAc to give 2-[2-[tert-butyl(diphenyl)silyl]oxyethoxy]acetaldehyde (510.0 mg, 1.49 mmol, 64.1% yield). MS [M+MeCN]⁺: 383.1

### Step 3: N-[4-[4-[(3aS,6aR)-2-[2-[2-[tert-butyl(diphenyl)silyl]oxyethoxy]ethyl]-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide

A solution of N-[4-[4-[(3aS,6aR)-2,3,3a,4,6,6a-hexahydro-1H-pyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (60.0 mg, 0.09 mmol, 1.0 eq), 2-[2-[tert-butyl(diphenyl)silyl]oxyethoxy]acetaldehyde (95.28 mg, 0.28 mmol, 3.0 eq), sodium acetate (22.82 mg, 0.28 mmol, 3.0 eq), and sodium cyanoborohydride (29.14 mg, 0.46 mmol, 5.0 eq) in methanol (1 mL) was stirred at 25° C. for 1 h. The resulting solution was concentrated under reduced pressure. The residue was applied on a silica gel column and eluted with 10:1 methylene chloride:MeOH to give N-[4-[4-[(3aS,6aR)-2-[2-[2-[tert-butyl(diphenyl)silyl]oxyethoxy]ethyl]-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (72.0 mg, 0.07 mmol, 62.2% yield) as a white solid. MS [M+H]⁺: 973.3

### Step 4: N-[4-[4-[(3aS,6aR)-2-[2-(2-hydroxyethoxy)ethyl]-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide

A solution of N-[4-[4-[(3aS,6aR)-2-[2-[2-[tert-butyl(diphenyl)silyl]oxyethoxy]ethyl]-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (62.0 mg, 0.06 mmol, 1.0 eq), and triethylamine trihydrofluoride (102.67 mg, 0.64 mmol, 10.0 eq) in THF (3 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 20-50) to afford N-[4-[4-[(3aS,6aR)-2-[2-(2-hydroxyethoxy)ethyl]-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (24.6 mg, 0.03 mmol, 51.7% yield). MS [M+H]⁺: 735.2

### Compound 142

### N-[4-[4-[(3aS,6aR)-2-(2-hydroxyethyl)-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide

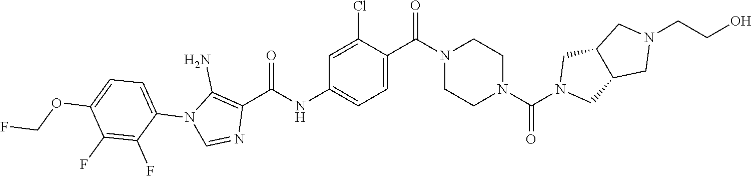 [SMILES: Nc1c(C(=O)Nc2ccc(C(=O)N3CCN(C(=O)N4C[C@H]5CN(CCO)C[C@H]5C4)CC3)c(Cl)c2)ncn1-c1ccc(OCF)c(F)c1F]

### Step 1: N-[4-[4-[(3aS,6aR)-2-[2-[tert-butyl(dimethyl)silyl]oxyethyl]-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide

A solution of N-[4-[4-[(3aS,6aR)-2,3,3a,4,6,6a-hexahydro-1H-pyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (60.0 mg, 0.09 mmol, 1.0 eq), (tert-butyldimethylsilyloxy) acetaldehyde (48.49 mg, 0.28 mmol, 3.0 eq), sodium acetate (22.82 mg, 0.28 mmol, 3.0 eq), sodium cyanoborohydride (29.14 mg, 0.46 mmol, 5.0 eq) in methanol (2 mL) was stirred at 25° C. for 1 h. The resulting solution was concentrated under reduced pressure. The residue was applied on a silica gel column and eluted with 10:1 methylene chloride:MeOH to give N-[4-[4-[(3aS,6aR)-2-[2-[tert-butyl(dimethyl)silyl]oxyethyl]-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (60.0 mg, 0.07 mmol, 80.3% yield). MS [M+H]⁺: 805.2

### Step 2: N-[4-[4-[(3aS,6aR)-2-(2-hydroxyethyl)-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide

A solution of N-[4-[4-[(3aS,6aR)-2-[2-[tert-butyl(dimethyl)silyl]oxyethyl]-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (50.0 mg, 0.06 mmol, 1.0 eq) and cesium fluoride (28.29 mg, 0.19 mmol, 3.0 eq) in MeCN (1 mL) was stirred at 25° C. for 4 h. The resulting solution was concentrated under reduced pressure. The crude product was purified by Prep-HPLC (Gemini C18 150×21.2 mm, 5 μm, MeCN-H₂O (0.1% TFA), 23-30) to afford N-[4-[4-[(3aS,6aR)-2-(2-hydroxyethyl)-1,3,3a,4,6,6a-hexahydropyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide (5.0 mg, 0.01 mmol, 11.65% yield). MS [M+H]⁺: 691.1

### Compound 143

### 5-amino-N-[3-chloro-4-[4-[1-(2-hydroxyethyl)piperidine-4-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

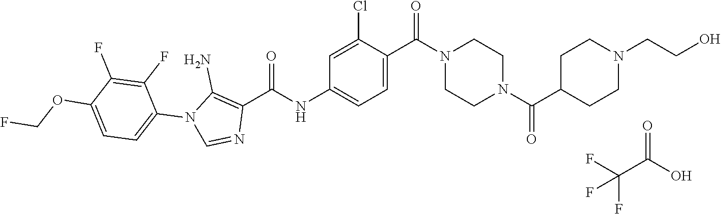

### Step 1: tert-butyl 4-[4-(2-chloro-4-nitrobenzoyl)piperazine-1-carbonyl]piperidine-1-carboxylate

A solution of (2-chloro-4-nitrophenyl)-piperazin-1-yl-methanone (1.3 g, 4.8 mmol, 1.0 eq), triphosgene (713.44 mg, 2.41 mmol, 0.5 eq), triethylamine (6.72 mL, 48.21 mmol, 10.0 eq), and 1-tert-butoxycarbonylpiperidine-4-carboxylic acid (0.9 g, 4.8 mmol, 1.0 eq) in methylene chloride (20 mL) was stirred at 20° C. for 1 h. The resulting solution was concentrated under reduced pressure. The residue was applied on a silica gel column and eluted with 99:1 ethyl acetate:petroleum ether to give tert-butyl 4-[4-(2-chloro-4-nitrobenzoyl)piperazine-1-carbonyl]piperidine-1-carboxylate (1.9 g, 3.9 mmol, 79% yield). MS [M+H]⁺: 481.0

### Step 2: tert-butyl 4-[4-(4-amino-2-chlorobenzoyl)piperazine-1-carbonyl]piperidine-1-carboxylate

A mixture of tert-butyl 4-[4-(2-chloro-4-nitrobenzoyl)piperazine-1-carbonyl]piperidine-1-carboxylate (1.5 g, 3.11 mmol, 1.0 eq), ammonium chloride (1.66 g, 31.1 mmol, 10.0 eq) and iron powder (1.74 g, 31.12 mmol, 10.0 eq) in ethanol (8 mL) and water (3 mL) was heated to 50° C. and reacted for 1 h. The solid was filtered out. The filtrate was concentrated under reduced pressure. The residue was diluted with water, then extracted with 2×50 mL of ethyl acetate. The organic layers were combined, washed with brine, dried and concentrated under reduced pressure to afford tert-butyl 4-[4-(4-amino-2-chlorobenzoyl)piperazine-1-carbonyl]piperidine-1-carboxylate (1.4 g, 3.1 mmol, 98% yield). MS [M+H]⁺: 451.0

### Step 3: 5-amino-1-[2,3-difluoro-4-(fluoromethoxy)phenyl]imidazole-4-carbonyl chloride

5-Amino-1-[2,3-difluoro-4-(fluoromethoxy)phenyl]imidazole-4-carboxylic acid (200.0 mg, 0.7 mmol, 1.0 eq) was added into thionyl chloride (2 mL) at 20° C. under N₂, and the solution was stirred at 20° C. for 1 hour under N₂. The resulting solution was evaporated to dryness with methylene chloride to afford the title compound 5-amino-1-[2,3-difluoro-4-(fluoromethoxy)phenyl]imidazole-4-carbonyl chloride (200.0 mg, 0.65 mmol, 84.8% yield), which was used in next step without further purification. MS [M−Cl+OCH₃+H]⁺: 302.1

### Step 4: tert-butyl 4-[4-[4-[[5-amino-1-[2,3-difluoro-4-(fluoromethoxy)phenyl]imidazole-4-carbonyl]amino]-2-chlorobenzoyl]piperazine-1-carbonyl]piperidine-1-carboxylate

To a solution of tert-butyl 4-[4-(4-amino-2-chlorobenzoyl)piperazine-1-carbonyl]piperidine-1-carboxylate (118.04 mg, 0.26 mmol, 0.8 eq) in methylene chloride (3 mL) were added pyridine (0.53 mL, 6.54 mmol, 20.0 eq) and 5-amino-1-[2,3-difluoro-4-(fluoromethoxy)phenyl]imidazole-4-carbonyl chloride (100.0 mg, 0.33 mmol, 1.0 eq). The reaction was stirred at 25° C. for 6 h and was then concentrated to dryness. The residue was taken up in methylene chloride (20 mL) and the organics washed with 2×20 mL water followed by 20 mL brine. The organics were then separated and dried (MgSO₄) before concentrating to dryness. The crude was then purified by flash column chromatography eluting with 8% MeOH in methylene chloride to afford tert-butyl 4-[4-[4-[[5-amino-1-[2,3-difluoro-4-(fluoromethoxy)phenyl]imidazole-4-carbonyl]amino]-2-chlorobenzoyl]piperazine-1-carbonyl]piperidine-1-carboxylate (150 mg, 0.21 mmol, 58% yield). MS [M−Boc+H]⁺: 620.1

### Step 5: 5-amino-N-[3-chloro-4-[4-(piperidine-4-carbonyl)piperazine-1-carbonyl]phenyl]-1-[2,3-difluoro-4-(fluoromethoxy)phenyl]imidazole-4-carboxamide

To a solution of tert-butyl 4-[4-[4-[[5-amino-1-[2,3-difluoro-4-(fluoromethoxy)phenyl]imidazole-4-carbonyl]amino]-2-chlorobenzoyl]piperazine-1-carbonyl]piperidine-1-carboxylate (150.0 mg, 0.21 mmol, 1.0 eq) in methylene chloride (3 mL) was added trifluoroacetic acid (1.0 mL). The reaction was stirred at 25° C. for 3 h. The reaction was concentrated to dryness to afford 5-amino-N-[3-chloro-4-[4-(piperidine-4-carbonyl)piperazine-1-carbonyl]phenyl]-1-[2,3-difluoro-4-(fluoromethoxy)phenyl]imidazole-4-carboxamide (120.0 mg, 0.19 mmol, 81.8% yield). MS [M+H]⁺: 620.2

### Step 6: 5-amino-N-[4-[4-[1-[2-[tert-butyl(dimethyl)silyl]oxyethyl]piperidine-4-carbonyl]piperazine-1-carbonyl]-3-chloro-phenyl]-1-[2,3-difluoro-4-(fluoromethoxy)phenyl]imidazole-4-carboxamide

A solution of 5-amino-N-[3-chloro-4-[4-(piperidine-4-carbonyl)piperazine-1-carbonyl]phenyl]-1-[2,3-difluoro-4-(fluoromethoxy)phenyl]imidazole-4-carboxamide (50.0 mg, 0.08 mmol, 1.0 eq), (tert-butyldimethylsilyloxy) acetaldehyde (42.17 mg, 0.24 mmol, 3.0 eq), sodium acetate (0.02 mL, 0.24 mmol, 3.0 eq), and sodium cyanoborohydride (25.34 mg, 0.4 mmol, 5.0 eq) in methanol (1 mL) was stirred at 25° C. for 3 h. The reaction was concentrated to dryness and the residue was taken up in EtOAc (20 mL) and the organics washed with 2×20 ml water and 20 mL brine. The organics were then separated and dried (MgSO₄) before concentrating to dryness to afford 5-amino-N-[4-[1-[2-[tert-butyl(dimethyl)silyl]oxyethyl]piperidine-4-carbonyl]piperazine-1-carbonyl]-3-chlorophenyl]-1-[2,3-difluoro-4-(fluoromethoxy)phenyl]imidazole-4-carboxamide (50.0 mg, 0.06 mmol, 73.2% yield). MS [M+H]⁺: 778.3

### Step 7: 5-amino-N-[3-chloro-4-[4-[1-(2-hydroxyethyl)piperidine-4-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

To a solution of 5-amino-N-[4-[4-[1-[2-[tert-butyl(dimethyl)silyl]oxyethyl]piperidine-4-carbonyl]piperazine-1-carbonyl]-3-chlorophenyl]-1-[2,3-difluoro-4-(fluoromethoxy)phenyl]imidazole-4-carboxamide (50.0 mg, 0.06 mmol, 1.0 eq) in THF (2 mL) added triethylamine trihydrofluoride (31.07 mg, 0.19 mmol, 3.0 eq). The reaction was stirred at 25° C. for 4 h. The reaction was concentrated to dryness to afford a crude product that was purified by Prep-HPLC (Gemini-C18 150×21.2 mm, 5 μm, MeCN-H₂O (0.1% TFA), 15-50) to afford 5-amino-N-[3-chloro-4-[4-[1-(2-hydroxyethyl)piperidine-4-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (20.0 mg, 0.03 mmol, 46.7% yield). MS [M+H]⁺: 664.2

### Compound 144

### 5-amino-N-[3-chloro-4-[4-[1-[2-(2-hydroxyethoxy)ethyl]piperidine-4-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

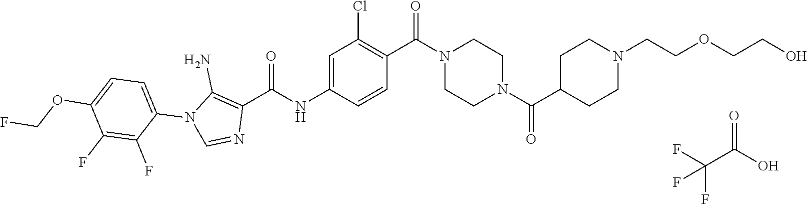 [SMILES: Nc1c(C(=O)Nc2ccc(C(=O)N3CCN(C(=O)C4CCN(CCOCCO)CC4)CC3)c(Cl)c2)ncn1-c1ccc(OCF)c(F)c1F ; O=C(O)C(F)(F)F]

### Step 1: 5-amino-N-[4-[4-[1-[2-[2-[tert-butyl(diphenyl)silyl]oxyethoxy]ethyl]piperidine-4-carbonyl]piperazine-1-carbonyl]-3-chlorophenyl]-1-[2,3-difluoro-4-(fluoromethoxy)phenyl]imidazole-4-carboxamide

A solution of 5-amino-N-[3-chloro-4-[4-(piperidine-4-carbonyl)piperazine-1-carbonyl]phenyl]-1-[2,3-difluoro-4-(fluoromethoxy)phenyl]imidazole-4-carboxamide (60.0 mg, 0.1 mmol, 1.0 eq), 2-[2-[tert-butyl(diphenyl)silyl]oxyethoxy]acetaldehyde (66.29 mg, 0.19 mmol, 2.0 eq) and sodium acetate (0.02 mL, 0.29 mmol, 3.0 eq), and sodium cyanoborohydride (30.41 mg, 0.48 mmol, 5.0 eq) in methanol (3 mL) was stirred at 25° C. for 3 h. The reaction was concentrated to dryness and the residue was taken up in methylene chloride (20 mL). The combined organics were washed with 2×20 mL water and 20 mL brine. The organics were then separated and dried (MgSO₄) before concentrating to dryness. to afford 5-amino-N-[1-[2-[tert-butyl(diphenyl)silyl]oxyethoxy]ethyl]piperidine-4-carbonyl]piperazine-1-carbonyl]-3-chlorophenyl]-1-[2,3-difluoro-4-(fluoromethoxy)phenyl]imidazole-4-carboxamide (55.0 mg, 0.06 mmol, 36% yield). MS [M+H]⁺: 946.2

### Step 2: 5-amino-N-[3-chloro-4-[4-[1-[2-(2-hydroxyethoxy)ethyl]piperidine-4-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

To a solution of 5-amino-N-[4-[4-[1-[2-[2-[tert-butyl(diphenyl)silyl]oxyethoxy]ethyl]piperidine-4-carbonyl]piperazine-1-carbonyl]-3-chlorophenyl]-1-[2,3-difluoro-4-(fluoromethoxy)phenyl]imidazole-4-carboxamide (55.0 mg, 0.06 mmol, 1.0 eq) in THF (2 mL) was added triethylamine trihydrofluoride (28.1 mg, 0.17 mmol, 3.0 eq), the reaction was stirred at 25° C. for 5 h. The reaction was concentrated to dryness. The crude was purified by Prep-HPLC (Gemini-C18 150×21.2 mm, 5 μm, MeCN-H₂O (0.1% TFA), 15-50) to afford 5-amino-N-[3-chloro-4-[4-[1-[2-(2-hydroxyethoxy)ethyl]piperidine-4-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (14.1 mg, 0.02 mmol, 29.2% yield). MS [M+H]⁺: 708.2

### Compound 145

### 5-amino-N-[3-chloro-4-[4-[4-(2-hydroxyethyl)piperazine-1-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

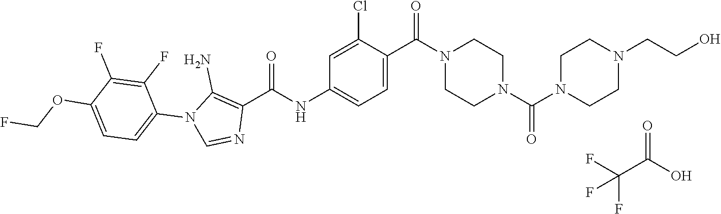

### Step 1: 5-amino-1-[2,3-difluoro-4-(fluoromethoxy)phenyl]imidazole-4-carbonyl chloride

A solution of 5-amino-1-[2,3-difluoro-4-(fluoromethoxy)phenyl]imidazole-4-carboxylic acid (200.0 mg, 0.7 mmol, 1.0 eq) and thionyl chloride (2 mL) was stirred at 20° C. for 1 hour under N₂. The resulting solution was evaporated to dryness with methylene chloride to afford the title compound 5-amino-1-[2,3-difluoro-4-(fluoromethoxy)phenyl]imidazole-4-carbonyl chloride (200.0 mg, 0.65 mmol, 84.8% yield), which was used in next step without further purification. LCMS: 302.1 [M−Cl+OCH₃+H]⁺

### Step 2: tert-butyl 4-[4-[4-[[5-amino-1-[2,3-difluoro-4-(fluoromethoxy)phenyl]imidazole-4-carbonyl]amino]-2-chlorobenzoyl]piperazine-1-carbonyl]piperazine-1-carboxylate

To a solution of tert-butyl 4-[4-(4-amino-2-chlorobenzoyl)piperazine-1-carbonyl]piperazine-1-carboxylate (118.3 mg, 0.26 mmol, 0.8 eq) in methylene chloride (3 mL) was added pyridine (0.53 mL, 6.54 mmol, 20.0 eq) and 5-amino-1-[2,3-difluoro-4-(fluoromethoxy)phenyl]imidazole-4-carbonyl chloride (100.0 mg, 0.33 mmol, 1.0 eq). The reaction was stirred at 25° C. for 6 h. The reaction was concentrated to dryness and the residue was taken up in methylene chloride (30 mL). These organics were washed with 2×20 ml water followed by 20 mL brine. The organics were separated and dried (MgSO₄) before concentrating to dryness. The crude was then purified by flash column chromatography eluting with 10% MeOH in methylene chloride to afford tert-butyl 4-[4-[4-[[5-amino-1-[2,3-difluoro-4-(fluoromethoxy)phenyl]imidazole-4-carbonyl]amino]-2-chlorobenzoyl]piperazine-1-carbonyl]piperazine-1-carboxylate (160.0 mg, 0.22 mmol, 67.8% yield). MS [M+H]⁺: 721.2

### Step 3: 5-amino-N-[3-chloro-4-[4-(piperazine-1-carbonyl)piperazine-1-carbonyl]phenyl]-1-[2,3-difluoro-4-(fluoromethoxy)phenyl]imidazole-4-carboxamide

To a solution of tert-butyl 4-[4-[4-[[5-amino-1-[2,3-difluoro-4-(fluoromethoxy)phenyl]imidazole-4-carbonyl]amino]-2-chlorobenzoyl]piperazine-1-carbonyl]piperazine-1-carboxylate (150.0 mg, 0.21 mmol, 1.0 eq) in methylene chloride (3 mL) was added trifluoroacetic acid (1.0 mL). The reaction was stirred at 25° C. for 3 h. The reaction was concentrated to dryness to afford 5-amino-N-[3-chloro-4-[4-(piperazine-1-carbonyl)piperazine-1-carbonyl]phenyl]-1-[2,3-difluoro-4-(fluoromethoxy)phenyl]imidazole-4-carboxamide (150.0 mg, 0.24 mmol, 84% yield). MS [M+H]⁺: 621.2

### Step 4: 5-amino-N-[4-[4-[4-[2-[tert-butyl(dimethyl)silyl]oxyethyl]piperazine-1-carbonyl]piperazine-1-carbonyl]-3-chlorophenyl]-1-[2,3-difluoro-4-(fluoromethoxy)phenyl]imidazole-4-carboxamide

A solution of 5-amino-N-[3-chloro-4-[4-(piperazine-1-carbonyl)piperazine-1-carbonyl]phenyl]-1-[2,3-difluoro-4-(fluoromethoxy)phenyl]imidazole-4-carboxamide (60.0 mg, 0.1 mmol, 1.0 eq), (tert-butyldimethylsilyloxy) acetaldehyde (50.52 mg, 0.29 mmol, 3.0 eq), sodium acetate (0.02 mL, 0.29 mmol, 3.0 eq), and sodium cyanoborohydride (30.36 mg, 0.48 mmol, 5.0 eq) in methanol (1 mL) was stirred at 25° C. for 1 h. The reaction was concentrated to dryness and the residue was taken up in EtOAc (20 mL) and the organics washed with 2×20 mL water followed by 20 mL brine. The organics were then separated and dried (MgSO₄) before concentrating to dryness to obtain 5-amino-N-[4-[4-[2-[tert-butyl(dimethyl)silyl]oxyethyl]piperazine-1-carbonyl]piperazine-1-carbonyl]-3-chlorophenyl]-1-[2,3-difluoro-4-(fluoromethoxy)phenyl]imidazole-4-carboxamide (50.0 mg, 0.06 mg, 0.06 mmol, 63.6% yield). MS [M+H]⁺: 779.3

### Step 5: 5-amino-N-[3-chloro-4-[4-[4-(2-hydroxyethyl)piperazine-1-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

To a solution of 5-amino-N-[4-[4-[4-[2-[tert-butyl(dimethyl)silyl]oxyethyl]piperazine-1-carbonyl]piperazine-1-carbonyl]-3-chlorophenyl]-1-[2,3-difluoro-4-(fluoromethoxy)phenyl]imidazole-4-carboxamide (50.0 mg, 0.06 mmol, 1.0 eq) in THF (2 mL) was added triethylamine trihydrofluoride (31.03 mg, 0.19 mmol, 3.0 eq). The reaction was stirred at 25° C. for 5 h and then concentrated to dryness. The crude material was purified by Prep-HPLC (Gemini-C18 150×21.2 mm, 5 μm, MeCN-H₂O (0.1% TFA), 15-50) to afford 5-amino-N-[3-chloro-4-[4-[4-(2-hydroxyethyl)piperazine-1-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (23.8 mg, 0.03 mmol, 47.4% yield). MS [M+H]⁺: 665.0

### Compound 146

### 5-amino-N-[3-chloro-4-[4-[4-[2-(2-hydroxyethoxy)ethyl]piperazine-1-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

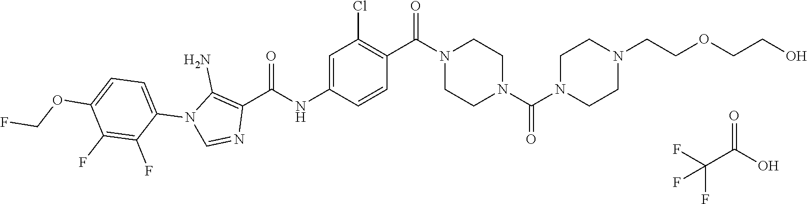 [SMILES: Nc1c(C(=O)Nc2ccc(C(=O)N3CCN(C(=O)N4CCN(CCOCCO)CC4)CC3)c(Cl)c2)ncn1-c1ccc(OCF)c(F)c1F ; O=C(O)C(F)(F)F]

### Step 1: 5-amino-N-[4-[4-[4-[2-[2-[tert-butyl(diphenyl)silyl]oxyethoxy]ethyl]piperazine-1-carbonyl]piperazine-1-carbonyl]-3-chlorophenyl]-1-[2,3-difluoro-4-(fluoromethoxy)phenyl]imidazole-4-carboxamide

A solution of 5-amino-N-[3-chloro-4-[4-(piperazine-1-carbonyl)piperazine-1-carbonyl]phenyl]-1-[2,3-difluoro-4-(fluoromethoxy)phenyl]imidazole-4-carboxamide (50.0 mg, 0.08 mmol, 1.0 eq), 2-[2-[tert-butyl(diphenyl)silyl]oxyethoxy]acetaldehyde (55.15 mg, 0.16 mmol, 2.0 eq), sodium acetate (0.02 mL, 0.24 mmol, 3.0 eq), and sodium cyanoborohydride (25.3 mg, 0.4 mmol, 5.0 eq) in methanol (3 mL) was stirred at 25° C. for 3 h. The reaction was concentrated to dryness and the residue was taken up in EtOAc (20 mL) and the organics washed with 2×20 mL water followed by 20 mL brine. The organics were then separated and dried (MgSO₄) before concentrating to dryness to afford 5-amino-N-[4-[4-[4-[2-[2-[tert-butyl(diphenyl)silyl]oxyethoxy]ethyl]piperazine-1-carbonyl]piperazine-1-carbonyl]-3-chlorophenyl]-1-[2,3-difluoro-4-(fluoromethoxy)phenyl]imidazole-4-carboxamide (53.0 mg, 0.06 mmol, 41% yield). MS [M+H]⁺: 947.2

### Step 2: 5-amino-N-[3-chloro-4-[4-[4-[2-(2-hydroxyethoxy)ethyl]piperazine-1-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

A solution of 5-amino-N-[4-[4-[4-[2-[2-[tert-butyl(diphenyl)silyl]oxyethoxy]ethyl]piperazine-1-carbonyl]piperazine-1-carbonyl]-3-chlorophenyl]-1-[2,3-difluoro-4-(fluoromethoxy)phenyl]imidazole-4-carboxamide (53.0 mg, 0.06 mmol, 1.0 eq) in THF (2 mL) was added triethylamine trihydrofluoride (27.05 mg, 0.17 mmol, 3.0 eq). The reaction was stirred at 25° C. for 2 h. The reaction was concentrated to dryness to afford a crude product The crude material was purified by Prep-HPLC (Gemini-C18 150×21.2 mm, 5 μm, MeCN-H₂O (0.1% TFA), 15-50) to afford 5-amino-N-[3-chloro-4-[4-[4-[2-(2-hydroxyethoxy)ethyl]piperazine-1-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (25.7 mg, 0.03 mmol, 55.4% yield). MS [M+H]⁺: 709.1

### Compound 147

### 5-amino-N-[3-chloro-4-[4-(piperazine-1-carbonyl)piperazine-1-carbonyl]phenyl]-1-[4-(difluoromethoxy)-2-fluoro-phenyl]imidazole-4-carboxamide; 1:1 2,2,2-trifluoroacetic acid

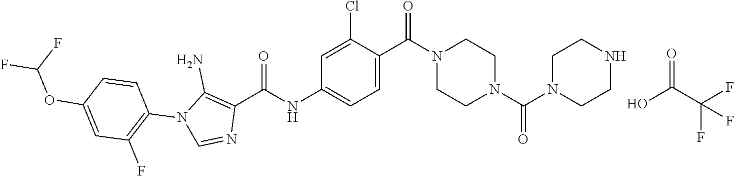

### Step 1: 4-(difluoromethoxy)-2-fluoro-1-nitrobenzene

To a mixture of 3-fluoro-4-nitrophenol (300.0 mg, 1.91 mmol, 1.0 eq) in MeCN (3 mL) were added N,N-diisopropylethylamine (987.19 mg, 7.64 mmol, 4.0 eq) and difluoroiodomethane (679.52 mg, 3.82 mmol, 2.0 eq). The vessel was sealed and the mixture was stirred at 25° C. After 15 h, the reaction mixture was diluted with EtOAc (30 mL), washed with H₂O (3×15 mL), dried over Na₂SO₄ and concentrated under reduced pressure. The resulting residue was purified by flash column chromatography (90:10 petroleum ether:EtOAc) to give 4-(difluoromethoxy)-2-fluoro-1-nitrobenzene (390.0 mg, 1.88 mmol, 98.6% yield).

### Step 2: 4-(difluoromethoxy)-2-fluoro-aniline

To a solution of 2-fluoro-4-difluoromethoxynitrobenzene (400.0 mg, 1.93 mmol, 1.0 eq) in methanol (15 mL) was added Pd/C (80.0 mg) under hydrogen atmosphere (balloon). The resulting mixture was stirred at 25° C. for 15 h. The solid was filtered off and the filtrate was concentrated under reduced pressure on 0° C. icewater bath to give 4-(difluoromethoxy)-2-fluoroaniline (320.0 mg, 1.81 mmol, 93.6% yield). MS [M+H]⁺: 178.1.

### Step 3: ethyl 5-amino-1-[4-(difluoromethoxy)-2-fluorophenyl]imidazole-4-carboxylate

To a solution of ethyl 2-amino-2-cyanoacetate; 4-methylbenzene-1-sulfonic acid (1085.2 mg, 3.61 mmol, 2.0 eq) in triethyl orthoformate (9.0 mL, 53.79 mmol, 29.77 eq) was added triethylamine (731.27 mg, 7.23 mmol, 4.0 eq). The resulting mixture was stirred at 83° C. for 2 h. The solvent was removed under reduced pressure and the residue was diluted with MeCN (7 mL), then 4-(difluoromethoxy)-2-fluoroaniline (320.0 mg, 1.81 mmol, 1.0 eq) was added. The resulting mixture was stirred at 40° C. for 15 h and then concentrated under reduced pressure. The resulting material was diluted with EtOAc (20 mL), washed with H₂O (3×15 mL), dried over Na₂SO₄, concentrated under reduced pressure and purified by flash column chromatography (60:40 petroleum ether:EtOAc) to give ethyl 5-amino-1-[4-(difluoromethoxy)-2-fluoro-phenyl]imidazole-4-carboxylate (550.0 mg, 1.74 mmol, 77.2% yield) as an orange solid. MS [M+H]⁺: 316.1

### Step 4: 5-amino-1-[4-(difluoromethoxy)-2-fluoro-phenyl]imidazole-4-carboxylic acid

To a solution of ethyl 5-amino-1-[4-(difluoromethoxy)-2-fluorophenyl]imidazole-4-carboxylate (550.0 mg, 1.74 mmol, 1.0 eq) in isopropyl alcohol (7.4 mL) were added sodium hydroxide (467.53 mg, 11.69 mmol, 6.7 eq) and water (3.7 mL) under N₂. The resulting mixture was stirred at 50° C. for 10 h. The solvent was removed under reduced pressure, then diluted with H₂O (15 mL), extracted with EtOAc (3×15 mL). The aqueous layer was adjusted pH to 5 with HCl (1 N), filtered and concentrated to give 5-amino-1-[4-(difluoromethoxy)-2-fluorophenyl]imidazole-4-carboxylic acid (200.0 mg, 0.7 mmol, 38.7% yield). MS [M+H]⁺: 288.0

### Step 5: 5-amino-1-[4-(difluoromethoxy)-2-fluorophenyl]imidazole-4-carbonyl chloride

5-Amino-1-[4-(difluoromethoxy)-2-fluorophenyl]imidazole-4-carboxylic acid (50.0 mg, 0.17 mmol, 1.0 eq) was dissolved in thionyl chloride (2.0 mL, 28 mmol, 160 eq) at 0° C. under N₂ atmosphere. The resulting mixture was stirred at 25° C. for 1 h. The solvent was removed under reduced pressure to give 5-amino-1-[4-(difluoromethoxy)-2-fluorophenyl]imidazole-4-carbonyl chloride (53.0 mg, 0.17 mmol, 66.7% yield). MS [M−Cl+OCH₃+H]⁺: 302.0

### Step 6: tert-butyl 4-[4-[4-[[5-amino-1-[4-(difluoromethoxy)-2-fluorophenyl]imidazole-4-carbonyl]amino]-2-chlorobenzoyl]piperazine-1-carbonyl]piperazine-1-carboxylate

To a solution of tert-butyl 4-[4-(4-amino-2-chlorobenzoyl)piperazine-1-carbonyl]piperazine-1-carboxylate (51.75 mg, 0.11 mmol, 0.7 eq) in methylene chloride (3 mL) were added pyridine (0.26 mL, 3.3 mmol, 20 eq) and 5-amino-1-[4-(difluoromethoxy)-2-fluorophenyl]imidazole-4-carbonyl chloride (50.0 mg, 0.16 mmol, 1.0 eq) under N₂ atmosphere. The resulting mixture was stirred at 25° C. for 1 h and then diluted with methylene chloride (20 mL). This solution was washed with H₂O (3×15 mL), dried over Na₂SO₄ and concentrated under reduced pressure. The resulting residue was purified by flash column chromatography (96:4 methylene chloride:MeOH) to give tert-butyl 4-[4-[4-[[5-amino-1-[4-(difluoromethoxy)-2-fluorophenyl]imidazole-4-carbonyl]amino]-2-chlorobenzoyl]piperazine-1-carbonyl]piperazine-1-carboxylate (120.0 mg, 0.17 mmol, 87.5% yield). MS [M+H]⁺: 721.3

### Step 7: 5-amino-N-[3-chloro-4-[4-(piperazine-1-carbonyl)piperazine-1-carbonyl]phenyl]-1-[4-(difluoromethoxy)-2-fluorophenyl]imidazole-4-carboxamide; 1:1 2,2,2-trifluoroacetic acid

To a solution of tert-butyl 4-[4-[4-[[5-amino-1-[4-(difluoromethoxy)-2-fluorophenyl]imidazole-4-carbonyl]amino]-2-chlorobenzoyl]piperazine-1-carbonyl]piperazine-1-carboxylate (60.0 mg, 0.08 mmol, 1.0 eq) in methylene chloride (1 mL) was added trifluoroacetic acid (2.0 mL, 25.96 mmol, 312.01 eq) under N₂ atmosphere. The resulting mixture was stirred at 25° C. for 1 h. The solvent was removed under reduced pressure, the residue was purified by prep-HPLC [Gemini-C18, 150×21.2 mm, 5 μm, MeCN-H₂O (0.1% TFA), 25%˜50%] to give 5-amino-N-[3-chloro-4-[4-(piperazine-1-carbonyl)piperazine-1-carbonyl]phenyl]-1-[4-(difluoromethoxy)-2-fluorophenyl]imidazole-4-carboxamide; 1:1 2,2,2-trifluoroacetic acid (33.8 mg, 0.05 mmol, 54.7% yield). MS [M+H]⁺: 621.2

### Compound 148

### 5-amino-N-[3-chloro-4-[4-(piperazine-1-carbonyl)piperazine-1-carbonyl]phenyl]-1-[4-(dimethylamino)-2,3-difluorophenyl]imidazole-4-carboxamide; 1;1 2,2,2-trifluoroacetate acid

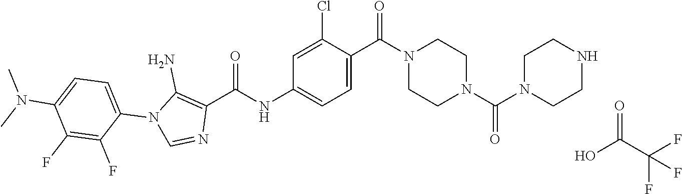

### Step 1: 4-bromo-2,3-difluoro-N,N-dimethylaniline

A solution of 4-bromo-2,3-difluoroaniline (2.0 g, 9.62 mmol, 1.0 eq), acetic acid (1.15 g, 19.2 mmol, 2.0 eq), polyformaldehyde (1.44 g, 48.1 mmol, 5.0 eq), and sodium cyanoborohydride (3.02 g, 48.1 mmol, 5.0 eq) in methanol (20 mL) was stirred at 50° C. for 16 h. The resulting solution was concentrated under reduced pressure. The residue was applied on a silica gel column and eluted with 30:1 petroleum ether:EtOAc to give 4-bromo-2,3-difluoro-N,N-dimethylaniline (1.2 g, 5.1 mmol, 50% yield). MS [M+H]⁺: 236.0

### Step 2: tert-butyl N-[4-(dimethylamino)-2,3-difluorophenyl]carbamate

A solution of 4-bromo-2,3-difluoro-N,N-dimethylaniline (1.1 g, 4.66 mmol, 1.0 eq), cesium carbonate (3.80 g, 11.65 mmol, 2.5 eq), tert-butyl carbamate (0.55 g, 4.66 mmol, 1.0 eq), tris(dibenzylideneacetonyl)bis-palladium (426.38 mg, 0.47 mmol, 0.1 eq) and Xantphos (269.34 mg, 0.47 mmol, 0.1 eq) in 1,4-dioxane (20 mL) was stirred at 85° C. for 16 h under N₂. The resulting solution was concentrated under reduced pressure. The residue was applied on a silica gel column and eluted with 30:1 petroleum ether:EtOAc to give tert-butyl N-[4-(dimethylamino)-2,3-difluorophenyl]carbamate (1.2 g, 2.2 mmol, 88% yield). MS [M−H]⁺: 273.0

### Step 3: 2,3-difluoro-N4,N4-dimethylbenzene-1,4-diamine

To a solution of tert-butyl N-[4-(dimethylamino)-2,3-difluoro-phenyl]carbamate (1.0 g, 3.67 mmol, 1.0 eq) in methylene chloride (10 mL) was added HCl (5 mL, 1M in 1,4-dioxane). The resulting mixture was stirred at 25° C. for 1 h. It was then concentrated under reduced pressure. The residue was applied on flash C18-m column (20-35 μm) and eluted with 1:2 H₂O:MeCN to give 2,3-difluoro-N4,N4-dimethylbenzene-1,4-diamine (300.0 mg, 1.74 mmol, 47% yield). MS [M+H]⁺: 173.0

### Step 4: ethyl 5-amino-1-[4-(dimethylamino)-2,3-difluorophenyl]imidazole-4-carboxylate

To a solution of ethyl 2-amino-2-cyanoacetate; 4-methylbenzene-1-sulfonic acid (837.25 mg, 2.79 mmol, 2.0 eq) in MeCN (5 mL) were added triethylamine (0.77 mL, 5.57 mmol, 4.0 eq) and triethyl orthoformate (2065.7 mg, 13.9 mmol, 10.0 eq). The resulting mixture was stirred at 80° C. for 2 h. The solvent was removed under reduced pressure and the residue was diluted with MeCN (5 mL), then 2,3-difluoro-N4,N4-dimethylbenzene-1,4-diamine (240.0 mg, 1.39 mmol, 1.0 eq) was added and the resulting mixture was stirred at 40° C. for 15 h. After concentration under reduced pressure, the residue was purified by flash column chromatography on silica gel (1:3 petroleum ether:EtOAc) to give ethyl 5-amino-1-[4-(dimethylamino)-2,3-difluorophenyl]imidazole-4-carboxylate (240.0 mg, 0.78 mmol, 51% yield). MS [M+H]⁺: 311.0

### Step 5: 5-amino-1-[4-(dimethylamino)-2,3-difluorophenyl]imidazole-4-carboxylic acid

Sodium hydroxide (128.9 mg, 3.22 mmol, 5.0 eq) was added to a solution of ethyl 5-amino-1-[4-(dimethylamino)-2,3-difluorophenyl]imidazole-4-carboxylate (200.0 mg, 0.64 mmol, 1.0 eq) in the isopropyl alcohol (3 mL) and water (1 mL), and the mixture was stirred at 50° C. for 12 h. The residue was diluted with 5 ml of water, then adjusted to pH=5˜6 with HCl (2M) and extracted with 3×5 mL of EtOAc. The organic layers were combined and concentrated under reduced pressure to give 5-amino-1-[4-(dimethylamino)-2,3-difluorophenyl]imidazole-4-carboxylic acid (120.0 mg, 0.43 mmol, 63. % yield). MS [M+H]⁺: 283.1

### Step 6: 5-amino-1-[4-(dimethylamino)-2,3-difluorophenyl]imidazole-4-carbonyl chloride

A solution of 5-amino-1-[4-(dimethylamino)-2,3-difluorophenyl]imidazole-4-carboxylic acid (90.0 mg, 0.32 mmol, 1.0 eq) in thionyl chloride (2.0 mL) was stirred at 25° C. for 1 h. The resulting solution was concentrated under reduced pressure to give 5-amino-1-[4-(dimethylamino)-2,3-difluorophenyl]imidazole-4-carbonyl chloride (80.0 mg, 0.27 mmol, 74. % yield). MS [M−Cl+OMe+H]⁺: 297.0

### Step 7: tert-butyl 4-[4-[4-[[5-amino-1-[4-(dimethylamino)-2,3-difluorophenyl]imidazole-4-carbonyl]amino]-2-chlorobenzoyl]piperazine-1-carbonyl]piperazine-1-carboxylate

A solution of tert-butyl 4-[4-(4-amino-2-chlorobenzoyl)piperazine-1-carbonyl]piperazine-1-carboxylate (90.18 mg, 0.2 mmol, 1.0 eq) and pyridine (0.16 mL, 2.0 mmol, 10.0 eq) in methylene chloride (2 mL) was stirred at 0° C., followed by addition of 5-amino-1-[4-(dimethylamino)-2,3-difluoro-phenyl]imidazole-4-carbonyl chloride (60.0 mg, 0.2 mmol, 1.0 eq) in methylene chloride (2 mL). The temperature was increased to 25° C. for 1 h. The resulting solution was concentrated under reduced pressure. The residue was applied on a silica gel column and eluted with 10:1 methylene chloride/MeOH to give tert-butyl 4-[4-[4-[[5-amino-1-[4-(dimethylamino)-2,3-difluorophenyl]imidazole-4-carbonyl]amino]-2-chloro-benzoyl]piperazine-1-carbonyl]piperazine-1-carboxylate (50.0 mg, 0.07 mmol, 26% yield). MS [M+H]⁺: 716.1

### Step 8: 5-amino-N-[3-chloro-4-[4-(piperazine-1-carbonyl)piperazine-1-carbonyl]phenyl]-1-[4-(dimethylamino)-2,3-difluorophenyl]imidazole-4-carboxamide; 1:1 2,2,2-trifluoroacetate acid

A solution of tert-butyl 4-[4-[4-[[5-amino-1-[4-(dimethylamino)-2,3-difluorophenyl]imidazole-4-carbonyl]amino]-2-chlorobenzoyl]piperazine-1-carbonyl]piperazine-1-carboxylate (50.0 mg, 0.07 mmol, 1.0 eq), and trifluoroacetic acid (1.0 mL) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN— H₂O (0.1% TFA), 15-40) to afford 5-amino-N-[3-chloro-4-[4-(piperazine-1-carbonyl)piperazine-1-carbonyl]phenyl]-1-[4-(dimethylamino)-2,3-difluorophenyl]imidazole-4-carboxamide;1:1 2,2,2-trifluoroacetate acid (13.7 mg, 0.02 mmol, 15.9% yield). MS [M+H]⁺: 616.2

### Compound 149

### 5-amino-N-[3-chloro-4-[4-(piperazine-1-carbonyl)piperazine-1-carbonyl]phenyl]-1-(2,3-difluoro-4-methoxyphenyl) imidazole-4-carboxamide; 1:1 2,2,2-trifluoroacetic acid

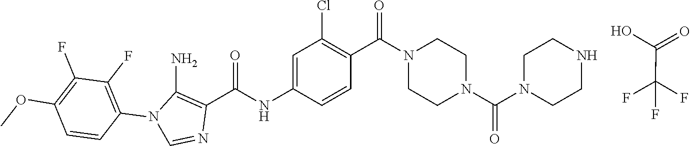

### Step 1: 2,3-difluoro-4-methoxyaniline

A solution of 2,3-difluoro-4-nitroanisole (4500.0 mg, 23.79 mmol, 1.0 eq), iron powder (6644.0 mg, 118.97 mmol, 5.0 eq) and ammonium chloride (6363.82 mg, 118.97 mmol, 5.0 eq) in ethanol (50 mL) and water (10 mL) was stirred at 50° C. for 3 h. The solid was filtered out. The crude was then purified by flash column chromatography eluting with with 40% EtOAc in petroleum ether to give the 2,3-difluoro-4-methoxyaniline (3000.0 mg, 18.85 mmol, 79% yield). MS [M+H]⁺: 160.1.

### Step 2: ethyl 5-amino-1-(2,3-difluoro-4-methoxyphenyl) imidazole-4-carboxylate

To a solution of ethyl 2-amino-2-cyanoacetate; 4-methylbenzene-1-sulfonic acid (10191.55 mg, 33.93 mmol, 2.0 eq) in MeCN (30 mL) were added triethylamine (9.46 mL, 67.87 mmol, 4.0 eq) and triethyl orthoformate (75.13 mL, 449.06 mmol, 26.47 eq). The resulting mixture was stirred at 80° C. for 2 h. The solvent was removed under reduced pressure and the residue was diluted with MeCN (30 mL). Next 2,3-difluoro-4-methoxyaniline (2700.0 mg, 16.97 mmol, 1.0 eq) was added. The resulting mixture was stirred at 20° C. for 15 h. After concentration under reduced pressure, the residue was purified by flash column chromatography on silica gel (55:45 petroleum ether:EtOAc) to give ethyl 5-amino-1-(2,3-difluoro-4-methoxyphenyl) imidazole-4-carboxylate (3500.0 mg, 11.77 mmol, 60.6% yield). MS [M+H]⁺: 298.1.

### Step 3: 5-amino-1-(2,3-difluoro-4-methoxyphenyl) imidazole-4-carboxylic acid

A solution of sodium hydroxide (2354.67 mg, 58.87 mmol, 5.0 eq) was added to ethyl 5-amino-1-(2,3-difluoro-4-methoxyphenyl) imidazole-4-carboxylate (3500.0 mg, 11.77 mmol, 1.0 eq) in isopropyl alcohol (4 mL) and water (2 mL). It was stirred at 50° C. for 15 h. The mixture was adjusted to pH 5-6 with 2N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure give 5-amino-1-(2,3-difluoro-4-methoxyphenyl) imidazole-4-carboxylic acid (1800.0 mg, 6.69 mmol, 56.8% yield). MS [M+H]⁺: 270.1.

### Step 4: 5-amino-1-(2,3-difluoro-4-methoxyphenyl) imidazole-4-carbonyl chloride

A solution of 5-amino-1-(2,3-difluoro-4-methoxyphenyl) imidazole-4-carboxylic acid (70.0 mg, 0.26 mmol, 1.0 eq) in thionyl chloride (309.36 mg, 2.6 mmol, 10.0 eq) was stirred at 25° C. for 1 h. The resulting solution was concentrated under reduced pressure to give 5-amino-1-(2,3-difluoro-4-methoxyphenyl) imidazole-4-carbonyl chloride (70.0 mg, 0.24 mmol, 85% yield). MS [(M−Cl+OCH₃)+H]⁺: 284.0

### Step 5: tert-butyl 4-[4-[4-[[5-amino-1-(2,3-difluoro-4-methoxyphenyl) imidazole-4-carbonyl]amino]-2-chlorobenzoyl]piperazine-1-carbonyl]piperazine-1-carboxylate

To a solution of tert-butyl 4-[4-(4-amino-2-chlorobenzoyl)piperazine-1-carbonyl]piperazine-1-carboxylate (76.99 mg, 0.17 mmol, 0.7 eq) and pyridine (0.2 mL, 2.43 mmol, 10.0 eq) in methylene chloride (2 mL) were added 5-amino-1-(2,3-difluoro-4-methoxyphenyl) imidazole-4-carbonyl chloride (70.0 mg, 0.24 mmol, 1.0 eq). The resulting mixture was stirred at 25° C. for 3 h. The reaction was concentrated to dryness and the residue was taken up in EtOAc (10 mL) and the organics washed with 2×3 mL water and 3 ml brine. The organics were then separated and dried (Na₂SO₄) before concentrating to dryness. The crude was then purified by flash column chromatography eluting with 5% MeOH in methylene chloride to give tert-butyl 4-[4-[4-[[5-amino-1-(2,3-difluoro-4-methoxyphenyl) imidazole-4-carbonyl]amino]-2-chlorobenzoyl]piperazine-1-carbonyl]piperazine-1-carboxylate (120.0 mg, 0.17 mmol, 64% yield). MS [M+H]⁺: 703.2

### Step 6: 5-amino-N-[3-chloro-4-[4-(piperazine-1-carbonyl)piperazine-1-carbonyl]phenyl]-1-(2,3-difluoro-4-methoxyphenyl) imidazole-4-carboxamide; 1:1 2,2,2-trifluoroacetic acid

To a solution of tert-butyl N-[2-[2-[[4-[[5-amino-1-(2,3-difluoro-4-methoxyphenyl) imidazole-4-carbonyl]amino]-2-chlorobenzoyl]amino]ethoxy]ethyl]carbamate (120.0 mg, 0.2 mmol, 1.0 eq) and trifluoroacetic acid (0.32 mL, 4.21 mmol, 21.36 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA)) to give 5-amino-N-[3-chloro-4-[4-(piperazine-1-carbonyl)piperazine-1-carbonyl]phenyl]-1-(2,3-difluoro-4-methoxyphenyl) imidazole-4-carboxamide; 1:1 2,2,2-trifluoroacetic acid (46.1 mg, 0.06 mmol, 32.2% yield). MS [M+H]⁺: 603.1.

### Compound 150

### N-[(1s,3s)-3-[[4-[[5-amino-1-[2,3-difluoro-4-(fluoromethoxy)phenyl]imidazole-4-carbonyl]amino]-2-chlorobenzoyl]amino]cyclobutyl]piperazine-1-carboxamide; 1:1 2,2,2-trifluoroacetic acid

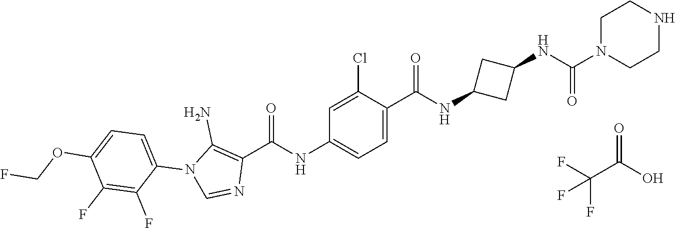 [SMILES: Nc1c(C(=O)Nc2ccc(C(=O)N[C@H]3C[C@@H](NC(=O)N4CCNCC4)C3)c(Cl)c2)ncn1-c1ccc(OCF)c(F)c1F ; O=C(O)C(F)(F)F]

### Step 1: tert-butyl N-(3-aminocyclobutyl) carbamate

To a solution of 2-chloro-4-nitrobenzoic acid (400.0 mg, 1.98 mmol, 1.0 eq) in methylene chloride (10 mL) were added tert-butyl N-(3-aminocyclobutyl) carbamate (369.62 mg, 1.98 mmol, 1.0 eq), o-(7-azabenzotriazol-1-yl)-N,N,N′,N′-tetramethyluronium hexafluorophosphate (1509.19 mg, 3.97 mmol, 2.0 eq) and N,N-diisopropylethylamine (768.01 mg, 5.95 mmol, 3.0 eq). The reaction was stirred at 25° C. for 15 h. The reaction was concentrated to dryness and the residue was taken up in methylene chloride (20 mL). The organics washed with 2×20 ml water followed by 20 mL brine. The organics were then separated and dried (MgSO₄) before concentrating to dryness. The crude was then purified by flash column chromatography eluting with 42% EtOAc in petroleum ether to afford tert-butyl N-[3-[(2-chloro-4-nitro-benzoyl)amino]cyclobutyl]carbamate (550.0 mg, 54.9% yield). MS [M−tBu+H]⁺: 314.00

### Step 2: N-(3-aminocyclobutyl)-2-chloro-4-nitrobenzamide

To a solution of tert-butyl N-[3-[(2-chloro-4-nitrobenzoyl)amino]cyclobutyl]carbamate (530.0 mg, 1.43 mmol, 1.0 eq) in methylene chloride (3 mL) was added HCl/dioxane (1.0 mL, 1.43 mmol, 1.0 eq). The reaction was stirred at 25° C. After 3 h the reaction was concentrated to dryness to afford a crude product N-(3-aminocyclobutyl)-2-chloro-4-nitrobenzamide (350.0 mg, 1.3 mmol, 73.2% yield). MS [M+H]⁺: 270.0

### Step 3: tert-butyl 4-[[3-[(2-chloro-4-nitrobenzoyl)amino]cyclobutyl]carbamoyl]piperazine-1-carboxylate

A solution of N-(3-aminocyclobutyl)-2-chloro-4-nitrobenzamide (350.0 mg, 1.3 mmol, 1.0 eq) in methylene chloride (3 mL) was added triphosgene (154.05 mg, 0.52 mmol, 0.4 eq). The reaction was stirred at 25° C. for 0.5 h. Triethylamine (2.71 mL, 19.47 mmol, 15.0 eq) and 1-Boc-piperazine (241.72 mg, 1.3 mmol, 1.0 eq) were then added and the reaction was stirred at 25° C. for 3 h. The reaction was concentrated to dryness and the residue was taken up in methylene chloride (10 mL). The organics were washed with 2×10 mL water followed by 10 mL brine. The organics were then separated and dried (MgSO₄) before concentrating to dryness. The crude was then purified by flash column chromatography eluting with 100% EtOAc to afford tert-butyl 4-[[3-[(2-chloro-4-nitrobenzoyl)amino]cyclobutyl]carbamoyl]piperazine-1-carboxylate (400.0 mg, 0.83 mmol, 50.8% yield). MS [M+H]⁺: 482.2

### Step 4: tert-butyl 4-[[3-[(4-amino-2-chlorobenzoyl)amino]cyclobutyl]carbamoyl]piperazine-1-carboxylate

To a solution of tert-butyl 4-[[3-[(2-chloro-4-nitrobenzoyl)amino]cyclobutyl]carbamoyl]piperazine-1-carboxylate (360.0 mg, 0.75 mmol, 1.0 eq) in ethanol (10 mL) and water (1 mL) were added iron powder (209.16 mg, 3.73 mmol, 5.0 eq) and ammonium chloride (199.79 mg, 3.73 mmol, 5.0 eq). The reaction was stirred at 50° C. After 5 h, the mixture was filtered through diatomaceous earth, and the filtrate was concentrated to afford the crude product tert-butyl 4-[[3-[(4-amino-2-chlorobenzoyl)amino]cyclobutyl]carbamoyl]piperazine-1-carboxylate (300.0 mg, 0.66 mmol, 66.3% yield). MS [M+H]⁺: 452.1

### Step 5: tert-butyl 4-[[3-[[4-[[5-amino-1-[2,3-difluoro-4-(fluoromethoxy)phenyl]imidazole-4-carbonyl]amino]-2-chlorobenzoyl]amino]cyclobutyl]carbamoyl]piperazine-1-carboxylate

To a solution of tert-butyl 4-[[3-[(4-amino-2-chlorobenzoyl)amino]cyclobutyl]carbamoyl]piperazine-1-carboxylate (73.94 mg, 0.16 mmol, 1.0 eq) in methylene chloride (3 mL) were added pyridine (0.26 mL, 3.27 mmol, 20.0 eq) and 5-amino-1-[2,3-difluoro-4-(fluoromethoxy)phenyl]imidazole-4-carbonyl chloride (50.0 mg, 0.16 mmol, 1.0 eq). The reaction was stirred 25° C. for 5 h. The reaction was concentrated to dryness and the residue was taken up in methylene chloride (15 mL). The organics washed with 2×10 mL water followed by 10 mL brine. The organics were then separated and dried (MgSO₄) before concentrating to dryness. The crude was then purified by flash column chromatography eluting with 12% MeOH in methylene chloride to afford tert-butyl 4-[[3-[[4-[[5-amino-1-[2,3-difluoro-4-(fluoromethoxy)phenyl]imidazole-4-carbonyl]amino]-2-chlorobenzoyl]amino]cyclobutyl]carbamoyl]piperazine-1-carboxylate (72.0 mg, 0.1 mmol, 54.2% yield). MS [M+H]⁺: 721.1

### Step 6: N-[3-[[4-[[5-amino-1-[2,3-difluoro-4-(fluoromethoxy)phenyl]imidazole-4-carbonyl]amino]-2-chlorobenzoyl]amino]cyclobutyl]piperazine-1-carboxamide; 1:1 2,2,2-trifluoroacetic acid

To a solution of tert-butyl 4-[[3-[[4-[[5-amino-1-[2,3-difluoro-4-(fluoromethoxy)phenyl]imidazole-4-carbonyl]amino]-2-chlorobenzoyl]amino]cyclobutyl]carbamoyl]piperazine-1-carboxylate (72.0 mg, 0.1 mmol, 1.0 eq) in methylene chloride (3 mL) was added trifluoroacetic acid (1.0 mL). The reaction was stirred at 25° C. After 3 h the reaction was concentrated to dryness to afford a crude product. The crude was purified by Prep-HPLC (Gemini-C18 150×21.2 mm, 5 μm, MeCN-H₂O (0.1% TFA), 20-40) to afford N-[3-[[4-[[5-amino-1-[2,3-difluoro-4-(fluoromethoxy)phenyl]imidazole-4-carbonyl]amino]-2-chlorobenzoyl]amino]cyclobutyl]piperazine-1-carboxamide; 1:1 2,2,2-trifluoroacetic acid (38.7 mg, 0.05 mmol, 62% yield). MS [M+H]⁺: 621.1

### Compound 151

### N-[4-[4-[(3aS,6aR)-2-methyl-1,3,3a,4,6,6a-hexahydropyrrolo[3,4-c]pyrrole-5-carbonyl]piperazine-1-carbonyl]-3-chlorophenyl]-5-amino-1-[1-(cyclobutylmethyl)-3-(trifluoromethyl)pyrazol-4-yl]imidazole-4-carboxamide; 1:1 2,2,2-trifluoroacetic acid

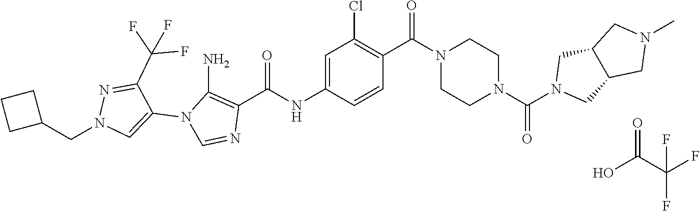 [SMILES: CN1C[C@@H]2CN(C(=O)N3CCN(C(=O)c4ccc(NC(=O)c5ncn(-c6cn(CC7CCC7)nc6C(F)(F)F)c5N)cc4Cl)CC3)C[C@@H]2C1 ; O=C(O)C(F)(F)F]

### Step 1: ethyl 5-amino-1-[3-(trifluoromethyl)-1H-pyrazol-4-yl]imidazole-4-carboxylate

A solution of 5-amino-1-[1-[(4-methoxyphenyl)methyl]-3-(trifluoromethyl)pyrazol-4-yl]imidazole-4-carboxylate (2.8 g, 6.84 mmol, 1.0 eq) in trifluoroacetic acid (20.0 mL, 259.6 mmol, 37.95 eq) was stirred at 70° C. for 8 h. The reaction was concentrated to dryness to afford ethyl 5-amino-1-[3-(trifluoromethyl)-1H-pyrazol-4-yl]imidazole-4-carboxylate (1.7 g, 5.88 mmol, 81.6% yield). MS [M+H]⁺: 290.0

### Step 2: 5-amino-1-[1-(cyclobutylmethyl)-3-(trifluoromethyl)pyrazol-4-yl]imidazole-4-carboxylate

To a solution of ethyl 5-amino-1-[3-(trifluoromethyl)-1H-pyrazol-4-yl]imidazole-4-carboxylate (1.65 g, 5.71 mmol, 1.0 eq) in DMF (20 mL) were added potassium carbonate (2.37 g, 17.12 mmol, 3.0 eq) and (bromomethyl)cyclobutene (1.02 g, 6.85 mmol, 1.2 eq) under N₂ atmosphere. The vessel was sealed, and the mixture was stirred at 25° for 15 h. The resulting solution was diluted with 100 ml of water, then extracted with 3×100 mL of EtOAc. The organic layers were combined, washed with brine, dried, and concentrated under reduced pressure. The crude product was purified by SFC separation to afford ethyl 5-amino-1-[1-(cyclobutylmethyl)-3-(trifluoromethyl)pyrazol-4-yl]imidazole-4-carboxylate (900.0 mg, 2.52 mmol, 44.15% yield). MS [M+H]⁺: 358.1.

### Step 3: 5-amino-1-[1-(cyclobutylmethyl)-3-(trifluoromethyl)pyrazol-4-yl]imidazole-4-carboxylic acid

A solution of ethyl 5-amino-1-[1-(cyclobutylmethyl)-3-(trifluoromethyl)pyrazol-4-yl]imidazole-4-carboxylate (850.0 mg, 2.38 mmol, 1.0 eq), sodium hydroxide (475.72 mg, 11.89 mmol, 5.0 eq) in isopropyl alcohol (10 mL) and water (5 mL) was stirred at 50° C. for 6 h. The residue was diluted with 100 ml of water, then adjusted to pH 6˜7 with HCl (2 M) and extracted with 3×10 mL of EtOAc. The organic layers were combined and concentrated under reduced pressure to afford 5-amino-1-[1-(cyclobutylmethyl)-3-(trifluoromethyl)pyrazol-4-yl]imidazole-4-carboxylic acid (630.0 mg, 1.91 mmol, 80.4% yield). MS [M+H]⁺: 330.1.

### Step 4: 5-amino-1-[1-(cyclobutylmethyl)-3-(trifluoromethyl)pyrazol-4-yl]imidazole-4-carbonyl chloride

5-Amino-1-[1-(cyclobutylmethyl)-3-(trifluoromethyl)pyrazol-4-yl]imidazole-4-carboxylic acid (100.0 mg, 0.3 mmol, 1.0 eq) was added into thionyl chloride (1.0 mL, 13.45 mmol, 44.28 eq). The mixture was stirred at 25° C. for 1 h. The resulting solution was concentrated under reduced pressure to give 5-amino-1-[1-(cyclobutylmethyl)-3-(trifluoromethyl)pyrazol-4-yl]imidazole-4-carbonyl chloride (100.0 mg, 0.29 mmol, 91.9% yield)(crude). MS [(M−Cl+OCH₃)+H]⁺: 344.2

### Step 5: tert-butyl (3aR,6aS)-2-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]-1,3,3a,4,6,6a-hexahydropyrrolo[3,4-c]pyrrole-5-carboxylate

A solution of 5-amino-1-[1-(cyclobutylmethyl)-3-(trifluoromethyl)pyrazol-4-yl]imidazole-4-carbonyl chloride (90.0 mg, 0.26 mmol, 1.0 eq), tert-butyl (3aR,6aS)-2-[4-(4-amino-2-chlorobenzoyl)piperazine-1-carbonyl]-1,3,3a,4,6,6a-hexahydropyrrolo[3,4-c]pyrrole-5-carboxylate (86.6 mg, 0.18 mmol, 0.7 eq) and pyridine (0.42 mL, 5.18 mmol, 20.0 eq) in methylene chloride (1 mL) was stirred at 25° C. for 1 h. The reaction was concentrated to dryness to afford crude product. The crude was then purified by flash column chromatography eluting with 95:5 methylene chloride:MeOH to give tert-butyl (3aR,6aS)-2-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (114.0 mg, 0.14 mmol, 43.0% yield). MS [(M−Boc)+H]⁺: 689.3.

### Step 6: N-[4-[4-[(3aS,6aR)-2,3,3a,4,6,6a-hexahydro-1H-pyrrolo[3,4-c]pyrrole-5-carbonyl]piperazine-1-carbonyl]-3-chloro-phenyl]-5-amino-1-[1-(cyclobutylmethyl)-3-(trifluoromethyl)pyrazol-4-yl]imidazole-4-carboxamide; 1:1 2,2,2-trifluoroacetic acid

A solution of tert-butyl (3aR,6aS)-2-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (104.0 mg, 0.13 mmol, 1.0 eq) and trifluoroacetic acid (1.0 mL) in methylene chloride (1 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40). The product was freeze-dried to afford N-[4-[4-[(3aS,6aR)-2,3,3a,4,6,6a-hexahydro-1H-pyrrolo[3,4-c]pyrrole-5-carbonyl]piperazine-1-carbonyl]-3-chlorophenyl]-5-amino-1-[1-(cyclobutylmethyl)-3-(trifluoromethyl)pyrazol-4-yl]imidazole-4-carboxamide; 1:1 2,2,2-trifluoroacetic acid (24.3 mg, 0.03 mmol, 26.4% yield). MS [M+H]⁺: 689.3.

### Step 7: N-[4-[4-[(3aS,6aR)-2-methyl-1,3,3a,4,6,6a-hexahydropyrrolo[3,4-c]pyrrole-5-carbonyl]piperazine-1-carbonyl]-3-chlorophenyl]-5-amino-1-[1-(cyclobutylmethyl)-3-(trifluoromethyl)pyrazol-4-yl]imidazole-4-carboxamide; 1:1 2,2,2-trifluoroacetic acid

A solution of N-[4-[4-[(3aS,6aR)-2,3,3a,4,6,6a-hexahydro-1H-pyrrolo[3,4-c]pyrrole-5-carbonyl]piperazine-1-carbonyl]-3-chlorophenyl]-5-amino-1-[1-(cyclobutylmethyl)-3-(trifluoromethyl)pyrazol-4-yl]imidazole-4-carboxamide (30.0 mg, 0.04 mmol, 1.0 eq), paraformadehyde (61.68 mg, 0.04 mmol, 1.0 eq), NaCNBH₃ (13.71 mg, 0.22 mmol, 5.0 eq) and NaOAc (10.71 mg, 0.13 mmol, 3.0 eq) in methanol (2 mL) was stirred at 25° C. for 2 h. The resulting solution was partitioned between water and ethyl acetate. The organic fraction was concentrated under reduced pressure at 30° C. The crude product was purified by Prep-HPLC (Gemini C18 150×21.2 mm, 5 μm, MeCN-H₂O (0.1% TFA), 15-40) to afford N-[4-[4-[(3aS,6aR)-2-methyl-1,3,3a,4,6,6a-hexahydropyrrolo[3,4-c]pyrrole-5-carbonyl]piperazine-1-carbonyl]-3-chlorophenyl]-5-amino-1-[1-(cyclobutylmethyl)-3-(trifluoromethyl)pyrazol-4-yl]imidazole-4-carboxamide; 1:1 2,2,2-trifluoroacetic acid (10.1 mg, 0.01 mmol, 32.5% yield). MS [M+H]⁺: 703.3.

### Compound 152

### 5-amino-N-[3-chloro-4-[4-(piperazine-1-carbonyl)piperidine-1-carbonyl]phenyl]-1-[1-(cyclobutylmethyl)-3-(trifluoromethyl)pyrazol-4-yl]imidazole-4-carboxamide; 1:1 2,2,2-trifluoroacetic acid

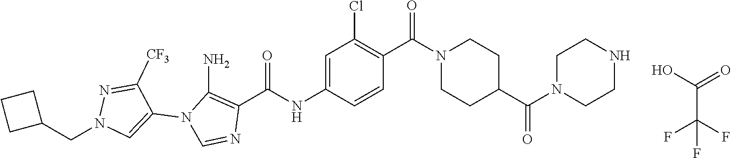 [SMILES: Nc1c(C(=O)Nc2ccc(C(=O)N3CCC(C(=O)N4CCNCC4)CC3)c(Cl)c2)ncn1-c1cn(CC2CCC2)nc1C(F)(F)F ; O=C(O)C(F)(F)F]

### Step 1: methyl 1-(2-chloro-4-nitrobenzoyl)piperidine-4-carboxylate

To a solution of 2-chloro-4-nitrobenzoic acid (1000.0 mg, 4.96 mmol, 1.0 eq) in methylene chloride (15 mL) were added methyl 4-piperidinecarboxylate (710.36 mg, 4.96 mmol, 1.0 eq), 2-(7-azabenzotriazol-1-yl)-N,N,N′,N′-tetramethyluronium hexafluorophosphate (3772.97 mg, 9.92 mmol, 2.0 eq) and N,N-diisopropylethylamine (2.59 mL, 14.88 mmol, 3.0 eq). The resulting mixture was stirred at 25° C. for 2 h. The reaction was then concentrated to dryness and the residue was taken up in EtOAc (50 ml). The organics washed with 2×10 mL water and 10 mL brine. The organics were then separated and dried (Na₂SO₄) before concentrating to dryness. The crude was then purified by flash column chromatography eluting with 60% EtOAc in petroleum ether to give methyl 1-(2-chloro-4-nitrobenzoyl)piperidine-4-carboxylate (1150.0 mg, 3.52 mmol, 70.9% yield). MS [M+H]⁺: 327.0

### Step 2: 1-(2-chloro-4-nitrobenzoyl)piperidine-4-carboxylic acid

Sodium hydroxide (673.29 mg, 16.83 mmol, 5.0 eq) was added to a solution of methyl 1-(2-chloro-4-nitrobenzoyl)piperidine-4-carboxylate (1100.0 mg, 3.37 mmol, 1.0 eq) in isopropyl alcohol (10 mL) and water (5 mL). The resulting mixture was stirred at 50° C. for 3 h. The mixture was adjusted to pH 5-6 with 2 N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure give 1-(2-chloro-4-nitrobenzoyl)piperidine-4-carboxylic acid (750.0 mg, 2.4 mmol, 71.2% yield). MS [M+H]⁺: 313.1.

### Step 3: tert-butyl 4-[1-(2-chloro-4-nitrobenzoyl)piperidine-4-carbonyl]piperazine-1-carboxylate

To a solution of 1-(2-chloro-4-nitrobenzoyl)piperidine-4-carboxylic acid (500.0 mg, 1.6 mmol, 1.0 eq) and 1-BOC-piperazine (297.8 mg, 1.6 mmol, 1.0 eq) in methylene chloride (3 mL) was added 2-(7-azabenzotriazol-1-yl)-N,N,N′,N′-tetramethyluronium hexafluorophosphate (1215.95 mg, 3.2 mmol, 2.0 eq) and N,N-diisopropylethylamine (0.84 mL, 4.8 mmol, 3.0 eq). The mixture was stirred at 25° C. for 2 h. The reaction was concentrated to dryness and the residue was taken up in EtOAc (10 ml). The organics was washed with 2×10 ml water followed by 10 ml brine. The organics was then separated and dried (MgSO₄) before concentrating to dryness. The crude was then purified by flash column chromatography eluting with 20% EtOAc in isohexane to give tert-butyl 4-[1-(2-chloro-4-nitrobenzoyl)piperidine-4-carbonyl]piperazine-1-carboxylate (538.0 mg, 1.12 mmol, 61.6% yield). MS [(M−C₄H₈)+H]⁺: 425.0

### Step 4: tert-butyl 4-[1-(4-amino-2-chlorobenzoyl)piperidine-4-carbonyl]piperazine-1-carboxylate

To a solution of tert-butyl 4-[1-(2-chloro-4-nitrobenzoyl)piperidine-4-carbonyl]piperazine-1-carboxylate (538.0 mg, 1.12 mmol, 1.0 eq) in ethanol (10 mL) were added iron powder (313.21 mg, 5.59 mmol, 5.0 eq), ammonium chloride (299.18 mg, 5.59 mmol, 5.0 eq) and water (1 mL) under N₂ atmosphere. The resulting mixture was stirred at 50° C. for 16 h. The solid was filtered out, and the filtrate was diluted with EtOAc (50 mL), washed with water (3×25 mL), dried over Na₂SO₄ and evaporated under reduced pressure to give tert-butyl 4-[1-(4-amino-2-chlorobenzoyl)piperidine-4-carbonyl]piperazine-1-carboxylate (403.0 mg, 0.89 mmol, 70.6% yield). MS [M+H]⁺: 451.0

### Step 5: 5-amino-1-[1-(cyclobutylmethyl)-3-(trifluoromethyl)pyrazol-4-yl]imidazole-4-carbonyl chloride

A solution of 5-amino-1-[1-(cyclobutylmethyl)-3-(trifluoromethyl)pyrazol-4-yl]imidazole-4-carboxylic acid (50.0 mg, 0.15 mmol, 1.0 eq) in thionyl chloride (180.65 mg, 1.52 mmol, 10.0 eq) was stirred at 25° C. for 1 h. The resulting solution was concentrated under reduced pressure to give 5-amino-1-[1-(cyclobutylmethyl)-3-(trifluoromethyl)pyrazol-4-yl]imidazole-4-carbonyl chloride (50.0 mg, 0.14 mmol, 73.2% yield). MS [(M−Cl+OCH₃)+H]⁺: 344.1.

### Step 6: tert-butyl 4-[1-[4-[[5-amino-1-[1-(cyclobutylmethyl)-3-(trifluoromethyl)pyrazol-4-yl]imidazole-4-carbonyl]amino]-2-chlorobenzoyl]piperidine-4-carbonyl]piperazine-1-carboxylate

To a solution of tert-butyl 4-[1-(4-amino-2-chlorobenzoyl)piperidine-4-carbonyl]piperazine-1-carboxylate (64.85 mg, 0.14 mmol, 1.0 eq) and pyridine (0.12 mL, 1.44 mmol, 10.0 eq) in methylene chloride (2 mL) were added 5-amino-1-[1-(cyclobutylmethyl)-3-(trifluoromethyl)pyrazol-4-yl]imidazole-4-carbonyl chloride (50.0 mg, 0.14 mmol, 1.0 eq). The resulting mixture was stirred at 25° C. for 3 h. The reaction was concentrated to dryness and the residue was taken up in EtOAc (10 ml) and the organics washed with 2×3 ml water followed by 3 ml brine. The organics were then separated and dried (Na₂SO₄) before concentrating to dryness. The crude was then purified by flash column chromatography eluting with 5% MeOH in methylene chloride to give tert-butyl 4-[1-[4-[[5-amino-1-[1-(cyclobutylmethyl)-3-(trifluoromethyl)pyrazol-4-yl]imidazole-4-carbonyl]amino]-2-chlorobenzoyl]piperidine-4-carbonyl]piperazine-1-carboxylate (80.0 mg, 0.1 mmol, 69.5% yield). MS [M+H]⁺: 762.2.

### Step 7: 5-amino-N-[3-chloro-4-[4-(piperazine-1-carbonyl)piperidine-1-carbonyl]phenyl]-1-[1-(cyclobutylmethyl)-3-(trifluoromethyl)pyrazol-4-yl]imidazole-4-carboxamide; 1:1 2,2,2-trifluoroacetic acid

A solution of tert-butyl 4-[1-[4-[[5-amino-1-[1-(cyclobutylmethyl)-3-(trifluoromethyl)pyrazol-4-yl]imidazole-4-carbonyl]amino]-2-chlorobenzoyl]piperidine-4-carbonyl]piperazine-1-carboxylate (60.0 mg, 0.08 mmol, 1.0 eq) and trifluoroacetic acid (1.0 mL, 12.98 mmol, 164.89 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA)) to give 5-amino-N-[3-chloro-4-[4-(piperazine-1-carbonyl)piperidine-1-carbonyl]phenyl]-1-[1-(cyclobutylmethyl)-3-(trifluoromethyl)pyrazol-4-yl]imidazole-4-carboxamide; 1:1 2,2,2-trifluoroacetic acid (20.8 mg, 0.03 mmol, 32.4% yield). MS [M+H]⁺: 662.3.

### Compound 153

### 5-amino-N-[3-chloro-4-[4-(piperazine-1-carbonyl)piperazine-1-carbonyl]phenyl]-1-[1-(cyclobutylmethyl)-3-(trifluoromethyl)pyrazol-4-yl]imidazole-4-carboxamide; 1:1 2,2,2-trifluoroacetic acid

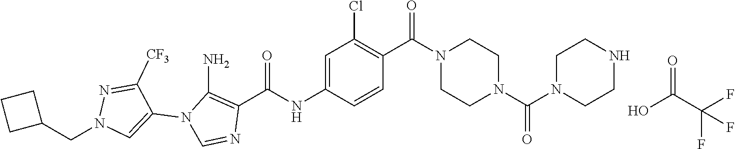

### Step 1: 5-amino-1-[1-(cyclobutylmethyl)-3-(trifluoromethyl)pyrazol-4-yl]imidazole-4-carbonyl chloride

A solution of 5-amino-1-[1-(cyclobutylmethyl)-3-(trifluoromethyl)pyrazol-4-yl]imidazole-4-carboxylic acid (50.0 mg, 0.15 mmol, 1.0 eq) in thionyl chloride (18.07 mg, 0.15 mmol, 1.0 eq) was stirred under N₂ atmosphere at 25° C. for 1 h. Concentrated under reduced pressure to give 5-amino-1-[1-(cyclobutylmethyl)-3-(trifluoromethyl)pyrazol-4-yl]imidazole-4-carbonyl chloride (52.0 mg, 0.15 mmol, 82.7% yield. MS [M−Cl+OCH₃+H]⁺: 344.0

### Step 2: tert-butyl 4-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]piperazine-1-carboxylate

To a solution of tert-butyl 4-[4-(4-amino-2-chlorobenzoyl)piperazine-1-carbonyl]piperazine-1-carboxylate (47.31 mg, 0.1 mmol, 0.7 eq) in methylene chloride (3 mL) were added pyridine (0.24 mL, 3.0 mmol, 20 eq) and 5-amino-1-[1-(cyclobutylmethyl)-3-(trifluoromethyl)pyrazol-4-yl]imidazole-4-carbonyl chloride (52.0 mg, 0.15 mmol, 1.0 eq) in order under N₂ atmosphere. The resulting mixture was stirred at 25° C. for 1 h. After this time, the reaction mixture was diluted with methylene chloride (20 mL), washed with H₂O (3×15 mL), dried over anhydrous Na₂SO₄, filtered and concentrated under reduced pressure. The residue was purified by flash column chromatography (92:8 methylene chloride:MeOH) to give tert-butyl 4-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]piperazine-1-carboxylate (100.0 mg, 0.13 mmol, 70.1% yield). MS [M+H]⁺: 763.0

### Step 3: 5-amino-N-[3-chloro-4-[4-(piperazine-1-carbonyl)piperazine-1-carbonyl]phenyl]-1-[1-(cyclobutylmethyl)-3-(trifluoromethyl)pyrazol-4-yl]imidazole-4-carboxamide; 1:1 2,2,2-trifluoroacetic acid

A solution of tert-butyl 4-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]piperazine-1-carboxylate (100.0 mg, 0.1 mmol, 1.0 eq) in trifluoroacetic acid (1.0 mL, 12.98 mmol, 123.83 eq) was stirred at 25° C. under N₂ atmosphere for 1 h. The solvent was removed under reduced pressure, the residue was purified by prep-HPLC [Gemini-C18, 150×21.2 mm, 5 μm, MeCN-H₂O (0.1% TFA), 20%˜45%] to give 5-amino-N-[3-chloro-4-[4-(piperazine-1-carbonyl)piperazine-1-carbonyl]phenyl]-1-[1-(cyclobutylmethyl)-3-(trifluoromethyl)pyrazol-4-yl]imidazole-4-carboxamide;2,2,2-trifluoroacetic acid (42.5 mg, 0.05 mmol, 51.6% yield). MS [M+H]⁺: 663.3

### Compound 154

### 5-amino-N-[3-chloro-4-[4-[(1R,5S,6r)-3-(2-hydroxyethyl)-3-azabicyclo[3.1.0]hexane-6-carbonyl]piperazine-1-carbonyl]phenyl]-1-[1-(cyclobutylmethyl)-3-(trifluoromethyl)pyrazol-4-yl]imidazole-4-carboxamide

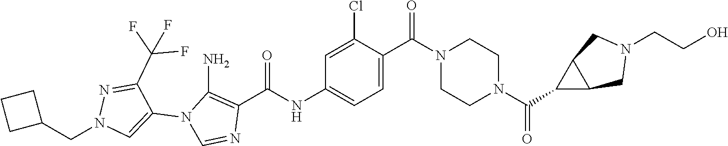 [SMILES: Nc1c(C(=O)Nc2ccc(C(=O)N3CCN(C(=O)[C@H]4[C@@H]5CN(CCO)C[C@@H]54)CC3)c(Cl)c2)ncn1-c1cn(CC2CCC2)nc1C(F)(F)F]

### Step 1: 5-amino-N-[4-[4-[(1R,5S)-3-[2-[tert-butyl(dimethyl)silyl]oxyethyl]-3-azabicyclo[3.1.0]hexane-6-carbonyl]piperazine-1-carbonyl]-3-chloro-phenyl]-1-[1-(cyclobutylmethyl)-3-(trifluoromethyl)pyrazol-4-yl]imidazole-4-carboxamide

A solution of 5-amino-N-[4-[4-[(1R,5S)-3-azabicyclo[3.1.0]hexane-6-carbonyl]piperazine-1-carbonyl]-3-chloro-phenyl]-1-[1-(cyclobutylmethyl)-3-(trifluoromethyl)pyrazol-4-yl]imidazole-4-carboxamide (60.0 mg, 0.09 mmol, 1.0 eq), (tert-butyldimethylsilyloxy) acetaldehyde (47.53 mg, 0.27 mmol, 3.0 eq), sodium acetate (22.37 mg, 0.27 mmol, 3.0 eq), sodium cyanoborohydride (28.56 mg, 0.45 mmol, 5.0 eq) in methanol (2 mL) was stirred at 25° C. for 1 h. The resulting solution was concentrated under reduced pressure. The residue was applied on a silica gel column and eluted with 10:1 methylene chloride/MeOH to give 5-amino-N-[4-[4-[(1R,5S)-3-[2-[tert-butyl(dimethyl)silyl]oxyethyl]-3-azabicyclo[3.1.0]hexane-6-carbonyl]piperazine-1-carbonyl]-3-chloro-phenyl]-1-[1-(cyclobutylmethyl)-3-(trifluoromethyl)pyrazol-4-yl]imidazole-4-carboxamide (60.0 mg, 0.07 mmol, 80.7% yield). MS [M+H]⁺: 818.2

### Step 2: 5-amino-N-[3-chloro-4-[4-[(1R,5S)-3-(2-hydroxyethyl)-3-azabicyclo[3.1.0]hexane-6-carbonyl]piperazine-1-carbonyl]phenyl]-1-[1-(cyclobutylmethyl)-3-(trifluoromethyl)pyrazol-4-yl]imidazole-4-carboxamide

A solution of 5-amino-N-[4-[4-[(1R,5S)-3-[2-[tert-butyl(dimethyl)silyl]oxyethyl]-3-azabicyclo[3.1.0]hexane-6-carbonyl]piperazine-1-carbonyl]-3-chlorophenyl]-1-[1-(cyclobutylmethyl)-3-(trifluoromethyl)pyrazol-4-yl]imidazole-4-carboxamide (60.0 mg, 0.07 mmol, 1.0 eq), triethylamine trihydrofluoride (118.19 mg, 0.73 mmol, 10.0 eq) in THF (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 20-50) to afford 5-amino-N-[3-chloro-4-[4-[(1R,5S)-3-(2-hydroxyethyl)-3-azabicyclo[3.1.0]hexane-6-carbonyl]piperazine-1-carbonyl]phenyl]-1-[1-(cyclobutylmethyl)-3-(trifluoromethyl)pyrazol-4-yl]imidazole-4-carboxamide; 2,2,2-trifluoroacetic acid (23.1 mg, 0.03 mmol, 44.6% yield). MS [M+H]⁺: 704.3

### Compound 155

### 5-amino-N-[3-chloro-4-[4-[(1S,5R,6r)-3-azabicyclo[3.1.0]hexane-6-carbonyl]piperazine-1-carbonyl]phenyl]-1-[1-cyclopropyl-3-(trifluoromethyl)pyrazol-4-yl]imidazole-4-carboxamide; 1:1 2,2,2-trifluoroacetic acid

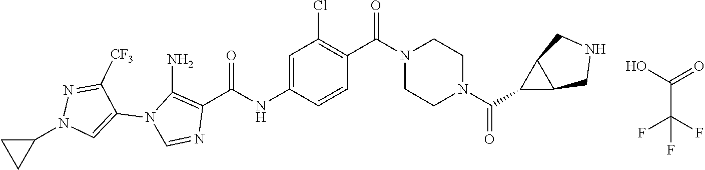 [SMILES: Nc1c(C(=O)Nc2ccc(C(=O)N3CCN(C(=O)[C@H]4[C@@H]5CNC[C@@H]54)CC3)c(Cl)c2)ncn1-c1cn(C2CC2)nc1C(F)(F)F ; O=C(O)C(F)(F)F]

### Step 1: 1-cyclopropyl-4-nitro-3-(trifluoromethyl)pyrazole

A solution of cupric acetate monohydrate (1543.66 mg, 7.73 mmol, 1.4 eq) and 2,2′-bipyridine (1207.56 mg, 7.73 mmol, 1.4 eq) in DCE (15 mL) was stirred at 80° C. for 30 min. The mixture was cooled to 25° C. 4-Nitro-3-(trifluoromethyl)-1H-pyrazole (1000.0 mg, 5.52 mmol, 1.0 eq), cyclopropylboronic acid (1897.61 mg, 22.09 mmol, 4.0 eq) and sodium carbonate (1463.36 mg, 13.81 mmol, 2.5 eq) were added to the reaction mixture and stirred at 80° C. for 16 h under O₂. The solid was filtered out. The crude was then purified by flash column chromatography eluting with 10% EtOAc in petroleum ether to give 1-cyclopropyl-4-nitro-3-(trifluoromethyl)pyrazole (300.0 mg, 1.36 mmol, 24.5% yield). MS [M+H]⁺: 222.0

### Step 2: 1-cyclopropyl-3-(trifluoromethyl)pyrazol-4-amine

To a solution of 1-cyclopropyl-4-nitro-3-(trifluoromethyl)pyrazole (300.0 mg, 1.36 mmol, 1.0 eq) in methanol (5 mL) was added palladium on carbon (60.0 mg) under hydrogen atmosphere (balloon). The resulting mixture was stirred at 25° C. for 2 h. The Pd/C was filtered off and the solvent was removed under reduced pressure to give 1-cyclopropyl-3-(trifluoromethyl)pyrazol-4-amine (210.0 mg, 1.1 mmol, 81.0% yield). MS [M+H]⁺: 192.0

### Step 3: ethyl 5-amino-1-[1-cyclopropyl-3-(trifluoromethyl)pyrazol-4-yl]imidazole-4-carboxylate

To a solution of ethyl 2-amino-2-cyanoacetate; 4-methylbenzene-1-sulfonic acid (628.47 mg, 2.09 mmol, 2.0 eq) in MeCN (30 mL) were added triethylamine (0.58 mL, 4.19 mmol, 4.0 eq) and triethyl orthoformate (4.63 mL, 27.7 mmol, 26.5 eq). The resulting mixture was stirred at 80° C. for 2 h. The solvent was removed under reduced pressure and the residue was diluted with MeCN (30 mL). 1-cyclopropyl-3-(trifluoromethyl)pyrazol-4-amine (200.0 mg, 1.05 mmol, 1.0 eq) was added and the resulting mixture was stirred at 50° C. for 15 h. After concentration under reduced pressure, the residue was purified by flash column chromatography on silica gel (30:70 petroleum ether:EtOAc) to give ethyl 5-amino-1-[1-cyclopropyl-3-(trifluoromethyl)pyrazol-4-yl]imidazole-4-carboxylate (200.0 mg, 0.61 mmol, 54.8% yield). MS [M+H]⁺: 330.0

### Step 4: 5-amino-1-[1-cyclopropyl-3-(trifluoromethyl)pyrazol-4-yl]imidazole-4-carboxylic acid

Sodium hydroxide (121.47 mg, 3.04 mmol, 5.0 eq) was added to a solution of ethyl 5-amino-1-[1-cyclopropyl-3-(trifluoromethyl)pyrazol-4-yl]imidazole-4-carboxylate (200.0 mg, 0.61 mmol, 1.0 eq) in isopropyl alcohol (4 mL) and water (2 mL). The resulting mixture was stirred at 50° C. for 15 h. The mixture was adjusted to pH 5-6 with 2 N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure give 5-amino-1-[1-cyclopropyl-3-(trifluoromethyl)pyrazol-4-yl]imidazole-4-carboxylic acid (100.0 mg, 0.33 mmol, 52.3% yield). MS [M+H]⁺: 302.0

### Step 5: 5-amino-1-[1-cyclopropyl-3-(trifluoromethyl)pyrazol-4-yl]imidazole-4-carbonyl chloride

A solution of 5-amino-1-[1-cyclopropyl-3-(trifluoromethyl)pyrazol-4-yl]imidazole-4-carboxylic acid (10.0 mg, 0.03 mmol, 1.0 eq) in thionyl chloride (39.5 mg, 0.33 mmol, 10.0 eq) was stirred at 25° C. for 1 h. The resulting solution was concentrated under reduced pressure to give 5-amino-1-[1-cyclopropyl-3-(trifluoromethyl)pyrazol-4-yl]imidazole-4-carbonyl chloride (10.0 mg, 0.03 mmol, 58.2% yield). MS [(M−Cl+OCH₃)+H]⁺: 316.2.

### Step 6: tert-butyl (1S,5R)-6-[4-[4-[[5-amino-1-[1-cyclopropyl-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl (1S,5R)-6-[4-(4-amino-2-chlorobenzoyl)piperazine-1-carbonyl]-3-azabicyclo[3.1.0]hexane-3-carboxylate (98.31 mg, 0.22 mmol, 1.0 eq) and pyridine (0.18 mL, 2.19 mmol, 10.0 eq) in methylene chloride (2 mL) was added 5-amino-1-[1-cyclopropyl-3-(trifluoromethyl)pyrazol-4-yl]imidazole-4-carbonyl chloride (70.0 mg, 0.22 mmol, 1.0 eq). The resulting mixture was stirred at 25° C. for 3 h. The reaction was concentrated to dryness and the residue was taken up in EtOAc (5 ml). The organics washed with 2×3 ml water followed by 3 ml brine. The organics were then separated and dried (Na₂SO₄) before concentrating to dryness. The crude was then purified by flash column chromatography eluting with 5% MeOH in methylene chloride to give tert-butyl (1S,5R)-6-[4-[4-[[5-amino-1-[1-cyclopropyl-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (100.0 mg, 0.14 mmol, 41.5% yield). MS [(M−Boc)+H]⁺: 632.0

### Step 7: 5-amino-N-[3-chloro-4-[4-[(1S,5R)-3-azabicyclo[3.1.0]hexane-6-carbonyl]piperazine-1-carbonyl]phenyl]-1-[1-cyclopropyl-3-(trifluoromethyl)pyrazol-4-yl]imidazole-4-carboxamide; 1:1 2,2,2-trifluoroacetic acid

To a solution of tert-butyl (1S,5R)-6-[4-[4-[[5-amino-1-[1-cyclopropyl-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (100.0 mg, 0.14 mmol, 1.0 eq) and trifluoroacetic acid (1.0 mL, 13 mmol, 95 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA), 25-40) to give 5-amino-N-[3-chloro-4-[4-[(1S,5R)-3-azabicyclo[3.1.0]hexane-6-carbonyl]piperazine-1-carbonyl]phenyl]-1-[1-cyclopropyl-3-(trifluoromethyl)pyrazol-4-yl]imidazole-4-carboxamide; 1:1 2,2,2-trifluoroacetic acid (34.3 mg, 0.05 mmol, 33.4% yield). MS [M+H]⁺: 631.2.

### Compound 156

### 5-amino-N-[4-[4-[(1S,5R,6r)-3-azabicyclo[3.1.0]hexane-6-carbonyl]piperazine-1-carbonyl]-3-chlorophenyl]-1-[1-[(3-fluorocyclobutyl)methyl]-3-(trifluoromethyl)pyrazol-4-yl]imidazole-4-carboxamide; 1:1 2,2,2-trifluoroacetic acid

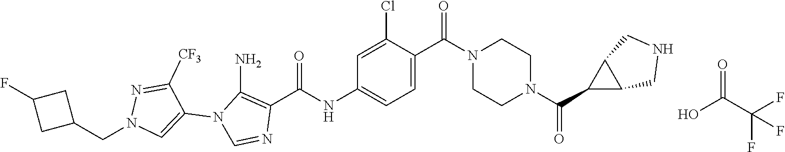 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2CC(F)C2)nc1C(F)(F)F ; O=C(O)C(F)(F)F]

### Step 1:(3-fluorocyclobutyl) methanol

To a solution of 3-fluorocyclobutanecarboxylic acid (1.0 g, 8.47 mmol, 1.0 eq) in Et₂O (10 mL) was added lithium aluminum hydride (0.32 g, 8.47 mmol, 1.0 eq). The reaction was stirred at 0° C. for 2 h then quenched by the addition of 0.03 mL of water. After addition of 0.03 mL 15% NaOH (aq.) and 0.09 mL water, the solid was filtered off. The filtrate was concentrated under reduced pressure to give (3-fluorocyclobutyl) methanol (600.0 mg, 5.76 mmol, 68.1% yield).

### Step 2: (3-fluorocyclobutyl)methyl 4-methylbenzenesulfonate

A solution of (3-fluorocyclobutyl) methanol (550.0 mg, 5.28 mmol, 1.0 eq), triethylamine (1.47 mL, 10.56 mmol, 2.0 eq), 4-dimethylaminopyridine (129.07 mg, 1.06 mmol, 0.2 eq) and p-toluenesulfonyl chloride (1208.5 mg, 6.34 mmol, 1.2 eq) in methylene chloride (1 mL) was stirred at 25° C. for 15 h. The resulting solution was concentrated under reduced pressure. The residue applied on a silica gel column and eluted with 1:2 EtOAc:petroleum ether to give (3-fluorocyclobutyl)methyl 4-methylbenzenesulfonate (1100.0 mg, 4.26 mmol, 80.6% yield). MS [M+Na]⁺: 281.0

### Step 3: tert-butyl (1S,5R)-6-[4-[4-[[5-amino-1-[1-[(3-fluorocyclobutyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of tert-butyl (1S,5R)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (400.0 mg, 0.58 mmol, 1.0 eq), (3-fluorocyclobutyl)methyl 4-methylbenzenesulfonate (223.94 mg, 0.87 mmol, 1.5 eq) and potassium carbonate (239.63 mg, 1.73 mmol, 3.0 eq) in DMF (6 mL). The resulting mixture was stirred at 25° C. for 2 h. The resulting solution was diluted with 10 mL of water, then extracted with 3×10 mL of EtOAc. The organic layers were combined, dried and concentrated under reduced pressure. The residue was applied on a silica gel column and eluted with 10:1 methylene chloride/MeOH to give tert-butyl (1S,5R)-6-[4-[4-[[5-amino-1-[1-[(3-fluorocyclobutyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (380.0 mg, 0.49 mmol, 84.5% yield). MS [M−tBu+H]⁺: 722.1

### Step 4: 5-amino-N-[4-[4-[(1S,5R)-3-azabicyclo[3.1.0]hexane-6-carbonyl]piperazine-1-carbonyl]-3-chlorophenyl]-1-[1-[(3-fluorocyclobutyl)methyl]-3-(trifluoromethyl)pyrazol-4-yl]imidazole-4-carboxamide; 1:2,2,2-trifluoroacetic acid

A solution of tert-butyl (1S,5R)-6-[4-[4-[[5-amino-1-[1-[(3-fluorocyclobutyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (60.0 mg, 0.08 mmol, 1.0 eq) and trifluoroacetic acid (1.0 mL, 13 mmol, 170 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 20-50) to afford 5-amino-N-[4-[4-[(1S,5R)-3-azabicyclo[3.1.0]hexane-6-carbonyl]piperazine-1-carbonyl]-3-chlorophenyl]-1-[1-[(3-fluorocyclobutyl)methyl]-3-(trifluoromethyl)pyrazol-4-yl]imidazole-4-carboxamide; 1:1 2,2,2-trifluoroacetic acid (38.1 mg, 0.05 mmol, 62.4% yield). MS [M+H]⁺: 678.1

### Compound 157

### 5-amino-N-[3-chloro-4-[4-[(1S,5R,6r)-3-methyl-3-azabicyclo[3.1.0]hexane-6-carbonyl]piperazine-1-carbonyl]phenyl]-1-[1-(3,3-difluoroallyl)-3-(trifluoromethyl)pyrazol-4-yl]imidazole-4-carboxamide

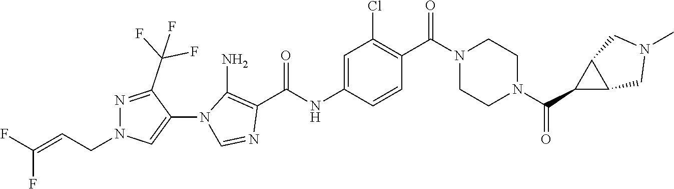 [SMILES: CN1C[C@@H]2[C@H](C1)[C@H]2C(=O)N1CCN(C(=O)c2ccc(NC(=O)c3ncn(-c4cn(CC=C(F)F)nc4C(F)(F)F)c3N)cc2Cl)CC1]

### Compound 158

### 5-amino-N-[4-[4-[(1S,5R,6r)-3-azabicyclo[3.1.0]hexane-6-carbonyl]piperazine-1-carbonyl]-3-chlorophenyl]-1-[1-(3,3-difluoroallyl)-3-(trifluoromethyl)pyrazol-4-yl]imidazole-4-carboxamide; 1:1 2,2,2-trifluoroacetic acid

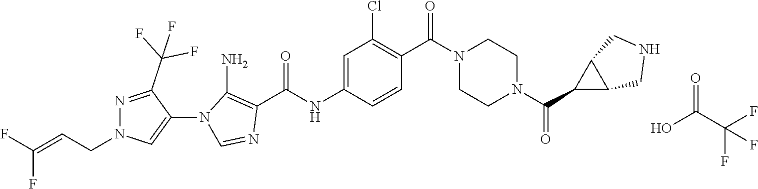 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC=C(F)F)nc1C(F)(F)F ; O=C(O)C(F)(F)F]

### Step 1: 3,3-difluoroallyl trifluoromethanesulfonate

A solution of 3,3-difluoroprop-2-en-1-ol (350.0 mg, 3.72 mmol, 1.0 eq), triethylamine (1.04 mL, 7.44 mmol, 2.0 eq), 4-dimethylaminopyridine (90.92 mg, 0.74 mmol, 0.2 eq) and trifluoromethanesulfonyl chloride (940.55 mg, 5.58 mmol, 1.5 eq) in methylene chloride (3 mL) was stirred at 25° C. for 1 h. The resulting solution was diluted with 5 ml of water then extracted with 2×5 mL of methylene chloride. The organic layers were combined and concentrated under reduced pressure to give 3,3-difluoroallyl trifluoromethanesulfonate (540.0 mg, 2.39 mmol, 64.2% yield).

### Step 2: tert-butyl (1S,5R)-6-[4-[4-[[5-amino-1-[1-(3,3-difluoroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of tert-butyl (1S,5R)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (180.0 mg, 0.26 mmol, 1.0 eq), 3,3-difluoroallyl trifluoromethanesulfonate (88.21 mg, 0.39 mmol, 1.5 eq) and potassium carbonate (107.83 mg, 0.78 mmol, 3.0 eq) in DMF (3 mL) was stirred at 25° C. for 2 h. The resulting solution was diluted with 10 mL of water then extracted with 3×10 mL of EtOAc. The organic layers were combined, dried and concentrated under reduced pressure. The residue was applied on a silica gel column and eluted with 10:1 methylene chloride/MeOH to give tert-butyl (1S,5R)-6-[4-[4-[[5-amino-1-[1-(3,3-difluoroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (152.0 mg, 0.2 mmol, 76.1% yield). MS [M−tBu+H]⁺: 712.1

### Step 3: 5-amino-N-[4-[4-[(1S,5R)-3-azabicyclo[3.1.0]hexane-6-carbonyl]piperazine-1-carbonyl]-3-chloro-phenyl]-1-[1-(3,3-difluoroallyl)-3-(trifluoromethyl)pyrazol-4-yl]imidazole-4-carboxamide; 1:1 2,2,2-trifluoroacetic acid

A solution of tert-butyl (1S,5R)-6-[4-[4-[[5-amino-1-[1-(3,3-difluoroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (152.0 mg, 0.2 mmol, 1.0 eq), trifluoroacetic acid (1.0 mL, 12.98 mmol, 65.59 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 30-60) to afford 5-amino-N-[4-[4-[(1S,5R)-3-azabicyclo[3.1.0]hexane-6-carbonyl]piperazine-1-carbonyl]-3-chlorophenyl]-1-[1-(3,3-difluoroallyl)-3-(trifluoromethyl)pyrazol-4-yl]imidazole-4-carboxamide; 1:1 2,2,2-trifluoroacetic acid (84.0 mg, 0.11 mmol, 53.8% yield). MS [M+H]⁺: 668.1

### Step 4: 5-amino-N-[3-chloro-4-[4-[(1S,5R)-3-methyl-3-azabicyclo[3.1.0]hexane-6-carbonyl]piperazine-1-carbonyl]phenyl]-1-[1-(3,3-difluoroallyl)-3-(trifluoromethyl)pyrazol-4-yl]imidazole-4-carboxamide

A solution of 5-amino-N-[4-[4-[(1S,5R)-3-azabicyclo[3.1.0]hexane-6-carbonyl]piperazine-1-carbonyl]-3-chlorophenyl]-1-[1-(3,3-difluoroallyl)-3-(trifluoromethyl)pyrazol-4-yl]imidazole-4-carboxamide;2,2,2-trifluoroacetic acid; 1:1 trifluoroacetic acid (65.0 mg, 0.1 mmol, 1.0 eq), polyformaldehyde (8.77 mg, 0.29 mmol, 3.0 eq), sodium acetate (23.95 mg, 0.29 mmol, 3.0 eq), and sodium cyanoborohydride (30.57 mg, 0.49 mmol, 5.0 eq) in methanol (3 mL) was stirred at 25° C. for 15 h. The resulting solution was concentrated under reduced pressure. The crude product was purified by Prep-HPLC (Gemini C18 150×21.2 mm, 5 μm, MeCN-H₂O (0.1% TFA), 20-40) to afford 5-amino-N-[3-chloro-4-[4-[(1S,5R)-3-methyl-3-azabicyclo[3.1.0]hexane-6-carbonyl]piperazine-1-carbonyl]phenyl]-1-[1-(3,3-difluoroallyl)-3-(trifluoromethyl)pyrazol-4-yl]imidazole-4-carboxamide (30.8 mg, 0.04 mmol, 39.8% yield). MS [M+H]⁺: 682.3

### Compound 159

### 5-amino-N-[3-chloro-4-[4-[(1S,5R,6r)-3-methyl-3-azabicyclo[3.1.0]hexane-6-carbonyl]piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid

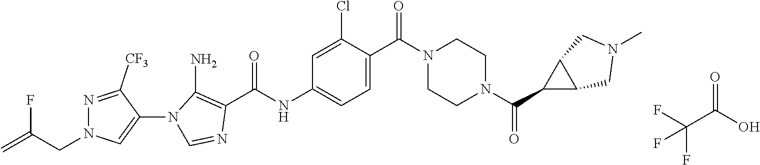 [SMILES: C=C(F)Cn1cc(-n2cnc(C(=O)Nc3ccc(C(=O)N4CCN(C(=O)[C@H]5[C@@H]6CN(C)C[C@@H]65)CC4)c(Cl)c3)c2N)c(C(F)(F)F)n1 ; O=C(O)C(F)(F)F]

### Step 1: 2-fluoroprop-2-en-1-ol

A solution of methyl 2-fluoroacrylate (500.0 mg, 4.8 mmol, 1.0 eq) in Et₂O (10 mL) was added lithium aluminum hydride (364.66 mg, 9.61 mmol, 2.0 eq). The reaction was stirred at 0° C. for 4 h. An excess of wet sodium sulphate was added to decompose excess lithium aluminum hydride. The solid phase was removed by filtration. The organics were then separated and dried (MgSO₄) before concentrating to dryness to afford 2-fluoroprop-2-en-1-ol (300.0 mg, 3.94 mmol, 41.0% yield).

### Step 2: 2-fluoroallyl 4-methylbenzenesulfonate

A solution of 2-fluoroprop-2-en-1-ol (150.0 mg, 1.97 mmol, 1.0 eq) in methylene chloride (5 mL) was added p-toluenesulfonyl chloride (375.94 mg, 1.97 mmol, 1.0 eq), triethylamine (0.82 mL, 5.92 mmol, 3.0 eq) and 4-dimethylaminopyridine (240.9 mg, 1.97 mmol, 1.0 eq). The reaction was stirred at 25° C. for 4 h. The reaction was concentrated to dryness to afford a crude product. The crude was purified by flash column chromatography eluting with 10% EtOAc in isohexane to afford 2-fluoroallyl 4-methylbenzenesulfonate (110.0 mg, 0.48 mmol, 22.5% yield). MS [M+H]⁺: 231.0

### Step 3: tert-butyl (1S,5R)-6-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of tert-butyl (1S,5R)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (130.0 mg, 0.19 mmol, 1.0 eq) in DMF (3 mL) was added 2-fluoroallyl 4-methylbenzenesulfonate (129.75 mg, 0.56 mmol, 3.0 eq) and potassium carbonate (77.88 mg, 0.56 mmol, 3.0 eq). The reaction was stirred at 25° C. for 10 h. The reaction was concentrated to dryness and the residue was taken up in EtOAc (20 mL) and the organics washed with 2×20 mL water followed by 20 mL brine. The organics were then separated and dried (MgSO₄) before concentrating to dryness. The crude was then purified by flash column chromatography eluting with 5% MeOH in methylene chloride to afford tert-butyl (1S,5R)-6-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (110.0 mg, 0.15 mmol, 75.5% yield). MS [M+H]⁺: 751.2

### Step 4: 5-amino-N-[3-chloro-4-[4-[(1S,5R)-3-azabicyclo[3.1.0]hexane-6-carbonyl]piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid

To a solution of tert-butyl (1S,5R)-6-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (100.0 mg, 0.13 mmol, 1.0 eq) in methylene chloride (3 mL) was added trifluoroacetic acid (1.0 mL). The reaction was stirred at 25° C. After 3 h the reaction mixture was concentrated to dryness to afford a crude product. The crude material was purified by Prep-HPLC (Gemini-C18 150×21.2 mm, 5 μm, MeCN-H₂O (0.1% TFA), 20-40) to afford 5-amino-N-[3-chloro-4-[4-[(1S,5R)-3-azabicyclo[3.1.0]hexane-6-carbonyl]piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid (80 mg, 0.10 mmol, 87.4% yield). MS [M+H]⁺: 650.2

### Step 5: 5-amino-N-[3-chloro-4-[4-[(1S,5R)-3-methyl-3-azabicyclo[3.1.0]hexane-6-carbonyl]piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid

A solution of 5-amino-N-[4-[4-[(1S,5R)-3-azabicyclo[3.1.0]hexane-6-carbonyl]piperazine-1-carbonyl]-3-chlorophenyl]-1-[1-(2-fluoroallyl)-3-(trifluoromethyl)pyrazol-4-yl]imidazole-4-carboxamide (40.0 mg, 0.06 mmol, 1.0 eq), polyformaldehyde (5.54 mg, 0.18 mmol, 3.0 eq), and sodium acetate (15.14 mg, 0.18 mmol, 3.0 eq), sodium cyanoborohydride (19.33 mg, 0.31 mmol, 5.0 eq) in methanol (3 mL) was stirred at 25° C. for 10 h. The resulting trifluoromethyl pyrazole was concentration under vaunted crude product was purified by Prep-HPLC (Gemini C18 150×21.2 mm, 5 μm, MeCN-H₂O (0.1% TFA), 20-40) to afford 5-amino-N-[3-chloro-4-[4-[(1S,5R)-3-methyl-3-azabicyclo[3.1.0]hexane-6-carbonyl]piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid (20.9 mg, 0.03 mmol, 43.4% yield). MS [M+H]⁺: 664.0

### Compound 160

### 5-amino-N-[3-chloro-4-[4-(4-methylpiperazine-1-carbonyl)piperazine-1-carbonyl]phenyl]-1-[2-(difluoromethyl)-3-fluoro-4-methoxyphenyl]imidazole-4-carboxamide; 1:1 2,2,2-trifluoroacetic acid

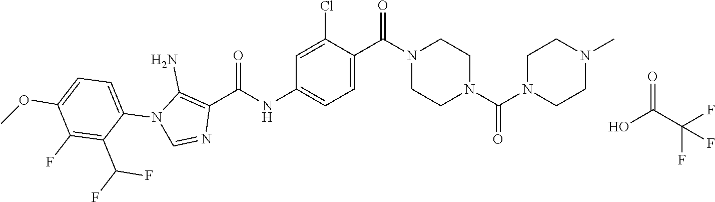

### Step 1: 5-amino-1-[2-(difluoromethyl)-3-fluoro-4-methoxyphenyl]imidazole-4-carbonyl chloride

A solution of 5-amino-1-[2-(difluoromethyl)-3-fluoro-4-methoxyphenyl]imidazole-4-carboxylic acid (100.0 mg, 0.33 mmol, 1.0 eq) in thionyl chloride (2.57 mL, 35.46 mmol, 106.81 eq) was stirred at 25° C. for 1 h. The resulting solution was concentrated under reduced pressure to afford 5-amino-1-[2-(difluoromethyl)-3-fluoro-4-methoxyphenyl]imidazole-4-carbonyl chloride (100.0 mg, 0.31 mmol, 94.2% yield). MS [(M−Cl+OCH₃)+H]⁺: 316.0

### Step 2: tert-butyl 4-[4-[4-[[5-amino-1-[2-(difluoromethyl)-3-fluoro-4-methoxyphenyl]imidazole-4-carbonyl]amino]-2-chlorobenzoyl]piperazine-1-carbonyl]piperazine-1-carboxylate

To a solution of tert-butyl 4-[4-(4-amino-2-chlorobenzoyl)piperazine-1-carbonyl]piperazine-1-carboxylate (141.38 mg, 0.31 mmol, 1.0 eq) and pyridine (0.46 mL, 5.63 mmol, 20.0 eq) in methylene chloride (2 mL) was added a suspension of 5-amino-1-[2-(difluoromethyl)-3-fluoro-4-methoxyphenyl]imidazole-4-carbonyl chloride (100.0 mg, 0.31 mmol, 1.0 eq). The mixture was stirred at 25° C. for 1 h under N₂. The resulting solution was evaporated under reduced pressure. The residue was purified by column chromatography (silica gel, 0 to 10% MeOH in methylene chloride) to give tert-butyl 4-[4-[4-[[5-amino-1-[2-(difluoromethyl)-3-fluoro-4-methoxyphenyl]imidazole-4-carbonyl]amino]-2-chlorobenzoyl]piperazine-1-carbonyl]piperazine-1-carboxylate (120.0 mg, 0.16 mmol, 52.2% yield). MS [M+H]⁺: 735.2.

### Step 3: 5-amino-N-[3-chloro-4-[4-(piperazine-1-carbonyl)piperazine-1-carbonyl]phenyl]-1-[2-(difluoromethyl)-3-fluoro-4-methoxyphenyl]imidazole-4-carboxamide

A solution of tert-butyl 4-[4-[4-[[5-amino-1-[2-(difluoromethyl)-3-fluoro-4-methoxyphenyl]imidazole-4-carbonyl]amino]-2-chlorobenzoyl]piperazine-1-carbonyl]piperazine-1-carboxylate (110.0 mg, 0.15 mmol, 1.0 eq) and trifluoroacetic acid (0.25 mL, 0.15 mmol, 1.0 eq) in methylene chloride (1 mL) was stirred at 25° C. for 1 h. The resulting solution was concentrated under reduced pressure. The crude product was purified by Prep-HPLC (Gemini-C18 150×21.2 mm, 5 μm, MeCN-H₂O (0.1% TFA), 15-50) to give 5-amino-N-[3-chloro-4-[4-(piperazine-1-carbonyl)piperazine-1-carbonyl]phenyl]-1-[2-(difluoromethyl)-3-fluoro-4-methoxyphenyl]imidazole-4-carboxamide (95.0 mg, 0.13 mmol, 84.8% yield). MS [M+H]⁺: 635.1.

### Step 4: 5-amino-N-[3-chloro-4-[4-(4-methylpiperazine-1-carbonyl)piperazine-1-carbonyl]phenyl]-1-[2-(difluoromethyl)-3-fluoro-4-methoxyphenyl]imidazole-4-carboxamide; 1:1 2,2,2-trifluoroacetic acid

To a solution of 5-amino-N-[3-chloro-4-[4-(piperazine-1-carbonyl)piperazine-1-carbonyl]phenyl]-1-[2-(difluoromethyl)-3-fluoro-4-methoxyphenyl]imidazole-4-carboxamide (50.0 mg, 0.08 mmol, 1.0 eq) and sodium acetate (0.02 mL, 0.24 mmol, 3.0 eq) in methanol (1 mL) was added polyformaldehyde (7.09 mg, 0.24 mmol, 3.0 eq) and sodium cyanoborohydride (24.74 mg, 0.39 mmol, 5.0 eq). The resulting mixture was stirred at 25° C. for 2 h. The solvent was then removed under reduced pressure, diluted with EtOAc (50 mL), washed with water (3×20 mL), dried over Na₂SO₄ and concentrated under reduced pressure. The crude product was purified by Prep-HPLC (Gemini-C18 150×21.2 mm, 5 μm, MeCN-H₂O (0.1% TFA), 35-40) to give 5-amino-N-[3-chloro-4-[4-(4-methylpiperazine-1-carbonyl)piperazine-1-carbonyl]phenyl]-1-[2-(difluoromethyl)-3-fluoro-4-methoxyphenyl]imidazole-4-carboxamide; 1:1 2,2,2-trifluoroacetic acid (49.2 mg, 0.06 mmol, 81.9% yield). MS [M+H]⁺: 649.0

### Compound 161

### 5-amino-N-[3-chloro-4-[4-(1-methylpiperidine-4-carbonyl)piperazine-1-carbonyl]phenyl]-1-[2-(difluoromethyl)-3-fluoro-4-methoxyphenyl]imidazole-4-carboxamide; 1:1 2,2,2-trifluoroacetic acid

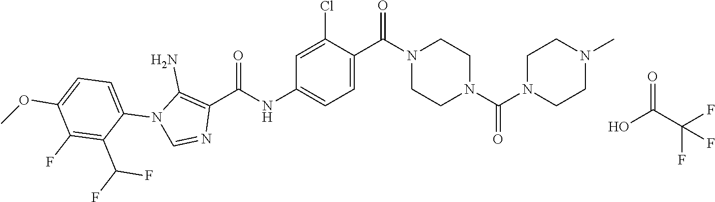

### Compound 162

### 5-amino-N-[3-chloro-4-[4-(piperidine-4-carbonyl)piperazine-1-carbonyl]phenyl]-1-[2-(difluoromethyl)-3-fluoro-4-methoxyphenyl]imidazole-4-carboxamide; 1:1 2,2,2-trifluoroacetic acid

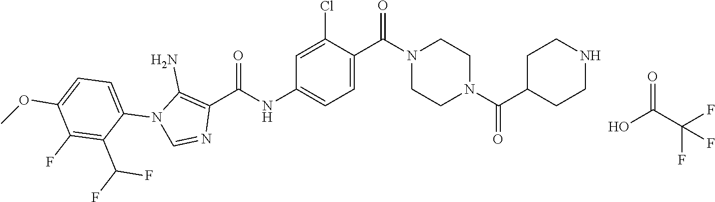 [SMILES: COc1ccc(-n2cnc(C(=O)Nc3ccc(C(=O)N4CCN(C(=O)C5CCNCC5)CC4)c(Cl)c3)c2N)c(C(F)F)c1F ; O=C(O)C(F)(F)F]

### Step 1: 5-amino-1-[2-(difluoromethyl)-3-fluoro-4-methoxyphenyl]imidazole-4-carbonyl chloride

5-Amino-1-[2-(difluoromethyl)-3-fluoro-4-methoxyphenyl]imidazole-4-carboxylic acid (90.0 mg, 0.3 mmol, 1.0 eq) was added into thionyl chloride (3.0 mL) at 25° C. under N₂, the solution was stirred at 25° C. for 3 h under N₂. The resulting solution was evaporated to dryness and azeotroped with methylene chloride to afford the title compound 5-amino-1-[2-(difluoromethyl)-3-fluoro-4-methoxyphenyl]imidazole-4-carbonyl chloride (90.0 mg, 0.28 mmol, 68.3% yield), which was used without further purification. MS [M−Cl+OCH₃+H]⁺: 316.0

### Step 2: tert-butyl 4-[4-[4-[[5-amino-1-[2-(difluoromethyl)-3-fluoro-4-methoxyphenyl]imidazole-4-carbonyl]amino]-2-chlorobenzoyl]piperazine-1-carbonyl]piperidine-1-carboxylate

To a solution of tert-butyl 4-[4-(4-amino-2-chlorobenzoyl)piperazine-1-carbonyl]piperidine-1-carboxylate (88.87 mg, 0.2 mmol, 0.7 eq) and pyridine (0.46 mL, 5.63 mmol, 20.0 eq) in methylene chloride (3.0 mL) was added a suspension of 5-amino-1-[2-(difluoromethyl)-3-fluoro-4-methoxyphenyl]imidazole-4-carbonyl chloride (90.0 mg, 0.28 mmol, 1.0 eq) in methylene chloride (3.0 mL) at 25° C. The mixture was stirred at 25° C. for 15 h under N₂. The resulting solution was evaporated under reduced pressure. The residue was purified by column chromatography (silica gel, 2% MeOH in methylene chloride) to afford tert-butyl 4-[4-[4-[[5-amino-1-[2-(difluoromethyl)-3-fluoro-4-methoxyphenyl]imidazole-4-carbonyl]amino]-2-chlorobenzoyl]piperazine-1-carbonyl]piperidine-1-carboxylate (150.0 mg, 0.2 mmol, 59.7% yield). MS [M+H−Boc]⁺: 634.0

### Step 3: 5-amino-N-[3-chloro-4-[4-(piperidine-4-carbonyl)piperazine-1-carbonyl]phenyl]-1-[2-(difluoromethyl)-3-fluoro-4-methoxyphenyl]imidazole-4-carboxamide; 1:1 2,2,2-trifluoroacetic acid

To a solution of tert-butyl 4-[4-[4-[[5-amino-1-[2-(difluoromethyl)-3-fluoro-4-methoxyphenyl]imidazole-4-carbonyl]amino]-2-chlorobenzoyl]piperazine-1-carbonyl]piperidine-1-carboxylate (140.0 mg, 0.19 mmol, 1.0 eq) in methylene chloride (3 mL) was added trifluoroacetic acid (1.0 mL), The reaction was stirred at 25° C. for 3 h. The reaction was concentrated to dryness to afford a crude product. The crude product was purified by Prep-HPLC (Gemini-C18 150×21.2 mm, 5 μm, MeCN-H₂O (0.1% TFA), 15-40) to afford 5-amino-N-[3-chloro-4-[4-(piperidine-4-carbonyl)piperazine-1-carbonyl]phenyl]-1-[2-(difluoromethyl)-3-fluoro-4-methoxyphenyl]imidazole-4-carboxamide; 1:1 2,2,2-trifluoroacetic acid (41.9 mg, 0.056 mmol, 69.8% yield). MS: [M+H]⁺: 633.9

### Step 4: 5-amino-N-[3-chloro-4-[4-(1-methylpiperidine-4-carbonyl)piperazine-1-carbonyl]phenyl]-1-[2-(difluoromethyl)-3-fluoro-4-methoxyphenyl]imidazole-4-carboxamide; 1:1 2,2,2-trifluoroacetic acid

A solution of 5-amino-N-[3-chloro-4-[4-(piperidine-4-carbonyl)piperazine-1-carbonyl]phenyl]-1-[2-(difluoromethyl)-3-fluoro-4-methoxyphenyl]imidazole-4-carboxamide (44.0 mg, 0.07 mmol, 1.0 eq), polyformaldehyde (6.25 mg, 0.21 mmol, 3.0 eq), and sodium acetate (0.02 mL, 0.21 mmol, 3.0 eq), and sodium cyanoborohydride (21.8 mg, 0.35 mmol, 5.0 eq) in methanol (3 mL) was stirred at 25° C. for 15 h. The resulting solution was concentrated under reduced pressure. The crude product was purified by Prep-HPLC (Gemini C18 150×21.2 mm, 5 μm, MeCN-H₂O (0.1% TFA), 20-40) to afford 5-amino-N-[3-chloro-4-[4-(1-methylpiperidine-4-carbonyl)piperazine-1-carbonyl]phenyl]-1-[2-(difluoromethyl)-3-fluoro-4-methoxyphenyl]imidazole-4-carboxamide; 1:1 2,2,2-trifluoroacetic acid (31.3 mg, 0.04 mmol, 59.2% yield). MS [M+H]⁺: 648.0

### Compound 163

### 5-amino-N-[-(3-chloro-4-[-(4-[(-((1R,5S,6r)-3-methyl-3-azabicyclo[3.1.0]hexane-6-carbonyl)piperazine-1-carbonyl)phenyl)-1-(2-(difluoromethyl)-3-fluoro-4-methoxyphenyl)-1H-imidazole-4-carboxamide; 1:1 2,2,2-trifluoroacetate

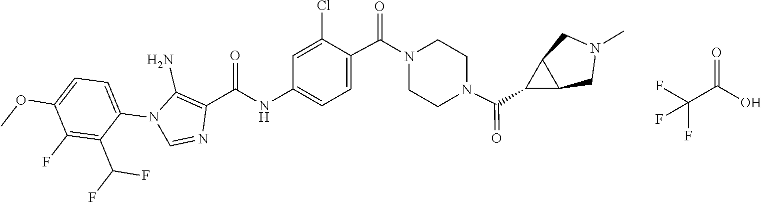

### Step 1: 5-amino-N-[3-chloro-4-[4-[(1R,5S)-3-methyl-3-azoniabicyclo[3.1.0]hexane-6-carbonyl]piperazine-1-carbonyl]phenyl]-1-[2-(difluoromethyl)-3-fluoro-4-methoxyphenyl]imidazole-4-carboxamide; 1:1 2,2,2-trifluoroacetate

A solution of 5-amino-N-[4-[4-[(1R,5S)-3-azabicyclo[3.1.0]hexane-6-carbonyl]piperazine-1-carbonyl]-3-chlorophenyl]-1-[2-(difluoromethyl)-3-fluoro-4-methoxyphenyl]imidazole-4-carboxamide (60.0 mg, 0.09 mmol, 1.0 eq), sodium cyanoborohydride (29.43 mg, 0.47 mmol, 5.0 eq), POM (14.24 mg, 0.47 mmol, 5.0 eq) and sodium acetate (0.04 mL, 0.47 mmol, 5.0 eq) in methanol (3 mL) was stirred at 20° C. for 6 h. The resulting solution was concentrated under a vacuum.

The residue was purified by Prep-HPLC (Gemini-C18 150×19 mm, 5 μm, MeCN-H₂O (0.1% TFA), 35-45) to afford 5-amino-N-[3-chloro-4-[4-[(1R,5S)-3-methyl-3-azoniabicyclo[3.1.0]hexane-6-carbonyl]piperazine-1-carbonyl]phenyl]-1-[2-(difluoromethyl)-3-fluoro-4-methoxyphenyl]imidazole-4-carboxamide; 1:1 2,2,2-trifluoroacetate (35.5 mg, 0.05 mmol, 49.0% yield). MS [M+H]⁺: 646.0

### Compound 164

### 5-amino-1-[2-(difluoromethyl)-3-fluoro-4-methoxyphenyl]-N-[3-fluoro-4-[4-[(1R,5S,6r)-3-methyl-3-azabicyclo[3.1.0]hexane-6-carbonyl]piperazine-1-carbonyl]phenyl]imidazole-4-carboxamide

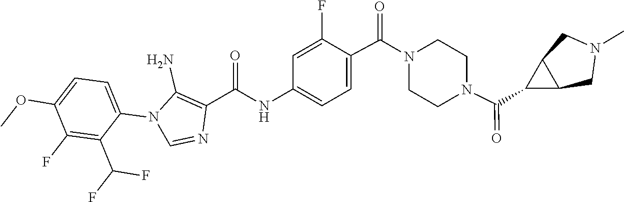

### Compound 165

### 5-amino-N-[4-[4-[(1R,5S,6r)-3-azabicyclo[3.1.0]hexane-6-carbonyl]piperazine-1-carbonyl]-3-fluorophenyl]-1-[2-(difluoromethyl)-3-fluoro-4-methoxyphenyl]imidazole-4-carboxamide; 1:1 2,2,2-trifluoroacetic acid

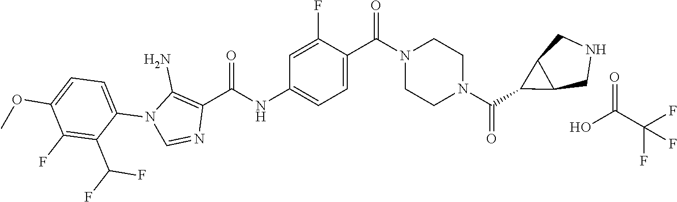

### Step 1: tert-butyl 4-(2-fluoro-4-nitrobenzoyl)piperazine-1-carboxylate

To a solution of 2-fluoro-4-nitrobenzoic acid (5.0 g, 27.01 mmol, 1.0 eq) in methylene chloride (100 mL) were added 1-BOC-piperazine (5.53 g, 29.71 mmol, 1.1 eq), propylphosphonic anhydride (34.38 g, 54.02 mmol, 2.0 eq) and N,N-diisopropylethylamine (10.47 g, 81.03 mmol, 3.0 eq). The resulting mixture was stirred at 25° C. for 1 h. The solvent was removed under reduced pressure, the residue was diluted with EtOAc (100 mL) and washed with H₂O (3×60 mL), dried over Na₂SO₄ and concentrated under reduced pressure to give tert-butyl 4-(2-fluoro-4-nitrobenzoyl)piperazine-1-carboxylate (8.0 g, 22.64 mmol, 83.8% yield). MS [M-t-Bu+H]⁺: 297.8

### Step 2: (2-fluoro-4-nitrophenyl) piperazin-1-yl-methanone

A solution of tert-butyl 4-(2-fluoro-4-nitrobenzoyl)piperazine-1-carboxylate (7.5 g, 21.23 mmol, 1.0 eq) in methylene chloride (50 mL) was added hydrogen chloride (30.0 mL, 1M in dioxane), then the mixture was stirred at 25° C. for 1 h. The resulting solution was concentrated under reduced pressure to give (2-fluoro-4-nitrophenyl) piperazin-1-yl-methanone (5.0 g, 19.74 mmol, 93.0% yield). MS [M+H]⁺: 253.9

### Step 3: tert-butyl (1S,5R)-6-[4-(2-fluoro-4-nitrobenzoyl)piperazine-1-carbonyl]-3-azabicyclo[3.1.0]hexane-3-carboxylate

A solution of (2-fluoro-4-nitrophenyl)-piperazin-1-yl-methanone (3.0 g, 12 mmol, 1.0 eq), (1S,5R)-3-tert-butoxycarbonyl-3-azabicyclo[3.1.0]hexane-6-carboxylic acid (2.96 g, 13.03 mmol, 1.1 eq), propylphosphonic anhydride (15.08 g, 23.69 mmol, 2.0 eq) and N,N-diisopropylethylamine (4.59 g, 35.54 mmol, 3.0 eq) in methylene chloride (30 mL) was stirred at 25° C. for 1 h. The resulting solution was diluted with 30 ml of water, then extracted with methylene chloride (2×30 mL). The organic layers were combined, washed with brine, dried and concentrated under reduced pressure. The residue was applied on a silica gel column and eluted with 1:1 petroleum ether:EtOAc to give tert-butyl (1S,5R)-6-[4-(2-fluoro-4-nitrobenzoyl)piperazine-1-carbonyl]-3-azabicyclo[3.1.0]hexane-3-carboxylate (4.5 g, 9.73 mmol, 82.1% yield). MS [M−t−Bu+H]⁺: 406.9

### Step 4: tert-butyl (1S,5R)-6-[4-(4-amino-2-fluorobenzoyl)piperazine-1-carbonyl]-3-azabicyclo[3.1.0]hexane-3-carboxylate

A solution of tert-butyl (1S,5R)-6-[4-(2-fluoro-4-nitrobenzoyl)piperazine-1-carbonyl]-3-azabicyclo[3.1.0]hexane-3-carboxylate (4.5 g, 9.73 mmol, 1.0 eq), ammonium chloride (2.6 g, 48.65 mmol, 5.0 eq) and Fe (2.72 g, 48.65 mmol, 5.0 eq) in ethanol (20 mL) and water (20 mL) was stirred at 60° C. for 4 h. The reaction mixture was quenched by the addition of brine. After quenching the reaction, the reaction mixture was poured into separatory funnel and separated. The residue was partitioned between ethyl acetate (100 mL). The organic layer was washed with water, dried (Na₂SO₄) and evaporated to dryness to afford tert-butyl (1S,5R)-6-[4-(4-amino-2-fluorobenzoyl)piperazine-1-carbonyl]-3-azabicyclo[3.1.0]hexane-3-carboxylate (3.3 g, 7.63 mmol, 78.4% yield). MS [M−Boc+H]⁺: 376.9

### Step 5: 5-amino-1-[2-(difluoromethyl)-3-fluoro-4-methoxyphenyl]imidazole-4-carbonyl chloride

A solution of 5-amino-1-[2-(difluoromethyl)-3-fluoro-4-methoxyphenyl]imidazole-4-carboxylic acid (100.0 mg, 0.33 mmol, 1.0 eq) in thionyl chloride (2.57 mL, 35.46 mmol, 106.81 eq) was stirred at 25° C. for 1 h. The resulting solution was concentrated under reduced pressure to give 5-amino-1-[2-(difluoromethyl)-3-fluoro-4-methoxyphenyl]imidazole-4-carbonyl chloride (100.0 mg, 0.31 mmol, 82.9% yield). MS [M−Cl+OMe+H]⁺: 316.0

### Step 6: tert-butyl (1R,5S)-6-[4-[4-[[5-amino-1-[2-(difluoromethyl)-3-fluoro-4-methoxyphenyl]imidazole-4-carbonyl]amino]-2-fluorobenzoyl]piperazine-1-carbonyl]-3-azabicyclo[3.1.0]hexane-3-carboxylate

A solution of tert-butyl (1R,5S)-6-[4-(4-amino-2-fluorobenzoyl)piperazine-1-carbonyl]-3-azabicyclo[3.1.0]hexane-3-carboxylate (94.7 mg, 0.22 mmol, 0.7 eq) and pyridine (0.51 mL, 6.26 mmol, 20.0 eq) in methylene chloride (2 mL) was stirred at 0° C. This was followed by the addition of 5-amino-1-[2-(difluoromethyl)-3-fluoro-4-methoxyphenyl]imidazole-4-carbonyl chloride (100.0 mg, 0.31 mmol, 1.0 eq) in methylene chloride (2 mL). The temperature was increased to 25° C. for 1 h. The resulting solution was concentrated under reduced pressure. The residue was applied on a silica gel column and eluted with 10:1 methylene chloride:MeOH to give tert-butyl (1R,5S)-6-[4-[4-[[5-amino-1-[2-(difluoromethyl)-3-fluoro-4-methoxyphenyl]imidazole-4-carbonyl]amino]-2-fluorobenzoyl]piperazine-1-carbonyl]-3-azabicyclo[3.1.0]hexane-3-carboxylate (140.0 mg, 0.2 mmol, 57.5% yield). MS [M+H]⁺: 716.2

### Step 7: 5-amino-N-[4-[4-[(1R,5S)-3-azabicyclo[3.1.0]hexane-6-carbonyl]piperazine-1-carbonyl]-3-fluoro-phenyl]-1-[2-(difluoromethyl)-3-fluoro-4-methoxyphenyl]imidazole-4-carboxamide; 1:1 2,2,2-trifluoroacetic acid

A solution of tert-butyl (1R,5S)-6-[4-[4-[[5-amino-1-[2-(difluoromethyl)-3-fluoro-4-methoxyphenyl]imidazole-4-carbonyl]amino]-2-fluorobenzoyl]piperazine-1-carbonyl]-3-azabicyclo[3.1.0]hexane-3-carboxylate (140.0 mg, 0.2 mmol, 1.0 eq), and trifluoroacetic acid (1 mL) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 20-50) to afford 5-amino-N-[4-[4-[(1R,5S)-3-azabicyclo[3.1.0]hexane-6-carbonyl]piperazine-1-carbonyl]-3-fluorophenyl]-1-[2-(difluoromethyl)-3-fluoro-4-methoxyphenyl]imidazole-4-carboxamide; 1:1 2,2,2-trifluoroacetic acid (90.0 mg, 0.12 mmol, 62.4% yield). MS [M+H]⁺: 616.2

### Step 8: 5-amino-1-[2-(difluoromethyl)-3-fluoro-4-methoxyphenyl]-N-[3-fluoro-4-[4-[(1R,5S)-3-methyl-3-azabicyclo[3.1.0]hexane-6-carbonyl]piperazine-1-carbonyl]phenyl]imidazole-4-carboxamide

A solution of 5-amino-N-[4-[4-[(1R,5S)-3-azabicyclo[3.1.0]hexane-6-carbonyl]piperazine-1-carbonyl]-3-fluoro-phenyl]-1-[2-(difluoromethyl)-3-fluoro-4-methoxyphenyl]imidazole-4-carboxamide (60.0 mg, 0.1 mmol, 1.0 eq), polyformaldehyde (8.78 mg, 0.29 mmol, 3.0 eq), sodium acetate (23.99 mg, 0.29 mmol, 3.0 eq), and sodium cyanoborohydride (30.62 mg, 0.49 mmol, 5.0 eq) in methanol (3 mL) was stirred at 25° C. for 15 h. The resulting solution was concentrated under reduced pressure. The crude product was purified by Prep-HPLC (Gemini C18 150×21.2 mm, 5 μm, MeCN-H₂O (0.1% TFA), 20-50) to afford 5-amino-1-[2-(difluoromethyl)-3-fluoro-4-methoxyphenyl]-N-[3-fluoro-4-[4-[(1R,5S)-3-methyl-3-azabicyclo[3.1.0]hexane-6-carbonyl]piperazine-1-carbonyl]phenyl]imidazole-4-carboxamide (29.8 mg, 0.04 mmol, 41.1% yield). MS [M+H]⁺: 630.2

### Compound 166

### N-(4-(4-((1R,5S,6r)-3-azabicyclo[3.1.0]hexane-6-carbonyl)piperazine-1-carbonyl)-3-chlorophenyl)-5-amino-1-(2-(difluoromethyl)-3-fluoro-4-methoxyphenyl)-1H-imidazole-4-carboxamide; 1:1 2,2,2-trifluoroacetate acid

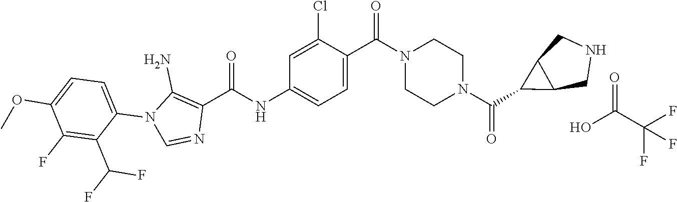

### Step 1: 5-amino-1-[2-(difluoromethyl)-3-fluoro-4-methoxyphenyl]imidazole-4-carbonyl chloride

A solution of 5-amino-1-[2-(difluoromethyl)-3-fluoro-4-methoxyphenyl]imidazole-4-carboxylic acid (50.0 mg, 0.17 mmol, 1.0 eq) in thionyl chloride (2.0 mL) was stirred at 25° C. for 1 h. The resulting solution was concentrated under reduced pressure to give 5-amino-1-[2-(difluoromethyl)-3-fluoro-4-methoxyphenyl]imidazole-4-carbonyl chloride (50.0 mg, 0.16 mmol, 76.3% yield). MS [M−Cl+OMe+H]⁺: 316.0

### Step 2: tert-butyl (1R,5S)-6-[4-[4-[[5-amino-1-[2-(difluoromethyl)-3-fluoro-4-methoxyphenyl]imidazole-4-carbonyl]amino]-2-chlorobenzoyl]piperazine-1-carbonyl]-3-azabicyclo[3.1.0]hexane-3-carboxylate

A solution of tert-butyl (1R,5S)-6-[4-(4-amino-2-chlorobenzoyl)piperazine-1-carbonyl]-3-azabicyclo[3.1.0]hexane-3-carboxylate (50.0 mg, 0.11 mmol, 0.71 eq) and pyridine (0.03 mL, 0.48 mmol, 3.0 eq) in methylene chloride (2 mL) was stirred at 0° C. This was followed by the addition of 5-amino-1-[2-(difluoromethyl)-3-fluoro-4-methoxyphenyl]imidazole-4-carbonyl chloride (50.0 mg, 0.16 mmol, 1.0 eq) in methylene chloride (2 mL). The temperature was increased to 25° C. for 1 h. The resulting solution was concentrated under reduced pressure. The residue was applied on a silica gel column and eluted with 10:1 methylene chloride:MeOH to give tert-butyl (1R,5S)-6-[4-[4-[[5-amino-1-[2-(difluoromethyl)-3-fluoro-4-methoxyphenyl]imidazole-4-carbonyl]amino]-2-chlorobenzoyl]piperazine-1-carbonyl]-3-azabicyclo[3.1.0]hexane-3-carboxylate (110.0 mg, 0.15 mmol, 96.0% yield). MS [M+H]⁺: 732.1

### Step 3: 5-amino-N-[4-[4-[(1R,5S)-3-azoniabicyclo[3.1.0]hexane-6-carbonyl]piperazine-1-carbonyl]-3-chloro-phenyl]-1-[2-(difluoromethyl)-3-fluoro-4-methoxyphenyl]imidazole-4-carboxamide; 1:1 2,2,2-trifluoroacetate acid

A solution of tert-butyl (1R,5S)-6-[4-[4-[[5-amino-1-[2-(difluoromethyl)-3-fluoro-4-methoxyphenyl]imidazole-4-carbonyl]amino]-2-chlorobenzoyl]piperazine-1-carbonyl]-3-azabicyclo[3.1.0]hexane-3-carboxylate (60.0 mg, 0.08 mmol, 1.0 eq), and trifluoroacetic acid (1.0 mL) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40%) to afford 5-amino-N-[4-[4-[(1R,5S)-3-azoniabicyclo[3.1.0]hexane-6-carbonyl]piperazine-1-carbonyl]-3-chlorophenyl]-1-[2-(difluoromethyl)-3-fluoro-4-methoxyphenyl]imidazole-4-carboxamide; 1:1 2,2,2-trifluoroacetate acid (31.5 mg, 0.04 mmol, 60.6% yield). MS [M+H]⁺: 632.2

### Compound 167

### 5-amino-N-[3-chloro-4-[4-[(3aS,6aR)-2-methyl-1,3,3a,4,6,6a-hexahydropyrrolo[3,4-c]pyrrole-5-carbonyl]piperazine-1-carbonyl]phenyl]-1-[2-(difluoromethyl)-3-fluoro-4-methoxyphenyl]imidazole-4-carboxamide; 1:1 2,2,2-trifluoroacetic acid

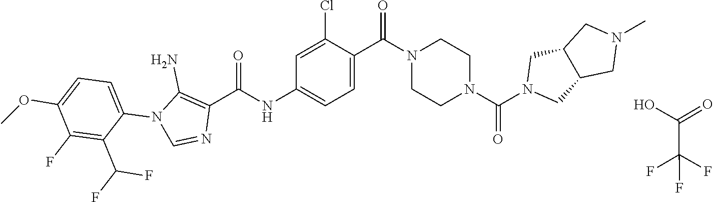

### Step 1: 2-fluoro-1-methoxy-3-methyl-4-nitrobenzene

A solution of 1,2-difluoro-3-methyl-4-nitrobenzene (10 g, 57.76 mmol, 1.0 eq) and sodium carbonate (18.3 g, 173.29 mmol, 3.0 eq) in methanol (120 mL) was stirred at 25° C. for 16 h. The reaction was concentrated to dryness and the residue was taken up in EtOAc (100 mL). The organics were washed with 2×100 ml water followed by 100 ml brine. The organics were then separated and dried (Na₂SO₄) before concentration to dryness to give 2-fluoro-1-methoxy-3-methyl-4-nitrobenzene (9000.0 mg, 48.61 mmol, 84.1% yield). MS [M+H]⁺: 186.1

### Step 2: 3-(bromomethyl)-2-fluoro-1-methoxy-4-nitrobenzene

To a solution of 2-fluoro-1-methoxy-3-methyl-4-nitrobenzene (8.4 g, 45.37 mmol, 1.0 eq) in carbon tetrachloride (60 mL) were added N-bromosuccinimide (12.1 g, 68.05 mmol, 1.5 eq) and benzoyl peroxide (1.1 g, 4.54 mmol, 0.1 eq). The resulting mixture was stirred at 80° C. for 36 h. The reaction was concentrated to dryness. The residue was taken up in EtOAc (200 ml) and the organics washed with 2×50 ml water followed by 50 ml brine. The organics were then separated and dried (Na₂SO₄) before concentrating to dryness. The crude was then purified by flash column chromatography eluting with 7% EtOAc in petroleum ether to give 3-(bromomethyl)-2-fluoro-1-methoxy-4-nitrobenzene (9000.0 mg, 34.08 mmol, 75.1% yield). MS [M+H]⁺: 265.0

### Step 3: 2-fluoro-3-methoxy-6-nitrobenzaldehyde

To a solution of 3-(bromomethyl)-2-fluoro-1-methoxy-4-nitrobenzene (9000.0 mg, 34.08 mmol, 1.0 eq) in MeCN (100 mL) were added N-methylmorpholine N-oxide (9582.86 mg, 81.8 mmol, 2.4 eq) and MOLECULAR SIEVES PACK 4A (1980.0 mg). The resulting mixture was stirred at 25° C. for 3 h. The reaction was concentrated to dryness and the residue was taken up in EtOAc (200 mL). The organics were washed with 2×50 mL water followed by 50 mL brine. The organics were then separated and dried (Na₂SO₄) before concentrating to dryness. The crude was then purified by flash column chromatography eluting with 7% EtOAc in petroleum ether to give 2-fluoro-3-methoxy-6-nitrobenzaldehyde (4700.0 mg, 23.6 mmol, 69.2% yield). MS [M+H]⁺: 200.1

### Step 4: 3-(difluoromethyl)-2-fluoro-1-methoxy-4-nitrobenzene

To a solution of 2-fluoro-3-methoxy-6-nitrobenzaldehyde (4400.0 mg, 22.1 mmol, 1.0 eq) in bis(2-methoxyethyl)aminosulfur trifluoride (22.0 mL, 118.33 mmol, 5.36 eq) was added methanol (110.61 mg, 2.21 mmol, 0.1 eq). The resulting mixture was stirred at 50° C. for 3 h. The reaction was concentrated to dryness. The residue was taken up in EtOAc (5 ml) and the organics washed with 2×3 ml water followed by 3 ml brine. The organics were then separated and dried (Na₂SO₄) before concentrating to dryness. The crude was then purified by flash column chromatography eluting with 7% EtOAc in petroleum ether to give 3-(difluoromethyl)-2-fluoro-1-methoxy-4-nitrobenzene (3200.0 mg, 14.47 mmol, 65.4% yield). MS [M+H]⁺: 221.0

### Step 5: 2-(difluoromethyl)-3-fluoro-4-methoxyaniline

A solution of 3-(difluoromethyl)-2-fluoro-1-methoxy-4-nitrobenzene (3200.0 mg, 14.47 mmol, 1.0 eq), iron (4040.7 mg, 72.36 mmol, 5.0 eq) and ammonium chloride (3870.3 mg, 72.36 mmol, 5.0 eq) in ethanol (30 mL) and water (6 mL) was stirred at 50° C. for 2 h. The solid was filtered off. The crude was then purified by flash column chromatography eluting with 40% EtOAc in petroleum ether to give the 2-(difluoromethyl)-3-fluoro-4-methoxyaniline (2000.0 mg, 10.46 mmol, 72.3% yield). MS [M+H]⁺: 192.1

### Step 6: ethyl 5-amino-1-[2-(difluoromethyl)-3-fluoro-4-methoxyphenyl]imidazole-4-carboxylate

To a solution of ethyl 2-amino-2-cyanoacetate; 4-methylbenzene-1-sulfonic acid (4713.52 mg, 15.69 mmol, 1.5 eq) in MeCN (30 mL) were added triethylamine (4.37 mL, 31.39 mmol, 3.0 eq) and triethyl orthoformate (46.33 mL, 276.92 mmol, 26.47 eq). The resulting mixture was stirred at 80° C. for 2 h. The solvent was removed under reduced pressure and the residue was diluted with MeCN (30 mL). Next, 2-(difluoromethyl)-3-fluoro-4-methoxyaniline (2000.0 mg, 10.46 mmol, 1.0 eq) was added and the resulting mixture was stirred at 50° C. for 15 h. After concentration under reduced pressure, the residue was purified by flash column chromatography on silica gel (30:70 petroleum ether:EtOAc) to give ethyl 5-amino-1-[2-(difluoromethyl)-3-fluoro-4-methoxyphenyl]imidazole-4-carboxylate (1700.0 mg, 5.16 mmol, 44.0% yield). MS [M+H]⁺: 330.0

### Step 7: 5-amino-1-[2-(difluoromethyl)-3-fluoro-4-methoxyphenyl]imidazole-4-carboxylic acid

Sodium hydroxide (789.57 mg, 19.74 mmol, 5.0 eq) was added into a solution of ethyl 5-amino-1-[2-(difluoromethyl)-3-fluoro-4-methoxyphenyl]imidazole-4-carboxylate (1300.0 mg, 3.95 mmol, 1.0 eq) in isopropyl alcohol (10 mL) and water (5 mL). The resulting mixture was stirred at 50° C. for 15 h. The mixture was adjusted to pH 5-6 with 2 N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure give 5-amino-1-[2-(difluoromethyl)-3-fluoro-4-methoxyphenyl]imidazole-4-carboxylic acid (860.0 mg, 2.86 mmol, 72.3% yield). MS [M+H]⁺: 302.2.

### Step 8: 5-amino-1-[2-(difluoromethyl)-3-fluoro-4-methoxyphenyl]imidazole-4-carbonyl chloride

A solution of 5-amino-1-[2-(difluoromethyl)-3-fluoro-4-methoxyphenyl]imidazole-4-carboxylic acid (95.0 mg, 0.32 mmol, 1.0 eq) in thionyl chloride (375.21 mg, 3.15 mmol, 10.0 eq) was stirred at 25° C. for 1 h. The resulting solution was concentrated under reduced pressure to give 5-amino-1-[2-(difluoromethyl)-3-fluoro-4-methoxyphenyl]imidazole-4-carbonyl chloride (95.0 mg, 0.3 mmol, 79.0% yield). MS [(M−Cl+OCH₃)+H]⁺: 316.0

### Step 9: tert-butyl (3aR,6aS)-2-[4-[4-[[5-amino-1-[2-(difluoromethyl)-3-fluoro-4-methoxyphenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate

To a solution of tert-butyl (3aR,6aS)-2-[4-(4-amino-2-chlorobenzoyl)piperazine-1-carbonyl]-1,3,3a,4,6,6a-hexahydropyrrolo[3,4-c]pyrrole-5-carboxylate (99.43 mg, 0.21 mmol, 0.7 eq) and pyridine (0.24 mL, 2.97 mmol, 10.0 eq) in methylene chloride (3 mL) were added 5-amino-1-[2-(difluoromethyl)-3-fluoro-4-methoxyphenyl]imidazole-4-carbonyl chloride (95.0 mg, 0.3 mmol, 1.0 eq). The resulting mixture was stirred at 25° C. for 3 h. The reaction was concentrated to dryness and the residue was taken up in EtOAc (5 ml) and the organics washed with 2×3 mL water and 3 mL brine. The organics were then separated and dried (Na₂SO₄) before concentrating to dryness. The crude was then purified by flash column chromatography eluting with 5% MeOH in methylene chloride to give tert-butyl (3aR,6aS)-2-[4-[4-[[5-amino-1-[2-(difluoromethyl)-3-fluoro-4-methoxyphenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (150.0 mg, 0.2 mmol, 66.3% yield). MS [M+H]⁺: 761.3

### Step 10: 5-amino-N-[3-chloro-4-[4-[(3aS,6aR)-2,3,3a,4,6,6a-hexahydro-1H-pyrrolo[3,4-c]pyrrole-5-carbonyl]piperazine-1-carbonyl]phenyl]-1-[2-(difluoromethyl)-3-fluoro-4-methoxyphenyl]imidazole-4-carboxamide; 1:1 2,2,2-trifluoroacetic acid

To a solution of tert-butyl (3aR,6aS)-2-[4-[4-[[5-amino-1-[2-(difluoromethyl)-3-fluoro-4-methoxyphenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (120.0 mg, 0.16 mmol, 1.0 eq) and trifluoroacetic acid (1.0 mL, 12.98 mmol, 82.33 eq) in methylene chloride (3 mL) was stirred at 25° C. for 1 h. The resulting solution was concentrated under reduced pressure to give 5-amino-N-[3-chloro-4-[4-[(3aS,6aR)-2,3,3a,4,6,6a-hexahydro-1H-pyrrolo[3,4-c]pyrrole-5-carbonyl]piperazine-1-carbonyl]phenyl]-1-[2-(difluoromethyl)-3-fluoro-4-methoxyphenyl]imidazole-4-carboxamide; 1:1 2,2,2-trifluoroacetic acid (80.0 mg, 0.1 mmol, 63.7% yield). MS [M+H]⁺: 661.3

### Step 11:5-amino-N-[3-chloro-4-[4-[(3aS,6aR)-2-methyl-1,3,3a,4,6,6a-hexahydropyrrolo[3,4-c]pyrrole-5-carbonyl]piperazine-1-carbonyl]phenyl]-1-[2-(difluoromethyl)-3-fluoro-4-methoxyphenyl]imidazole-4-carboxamide; 1:1 2,2,2-trifluoroacetic acid

A solution of 5-amino-N-[3-chloro-4-[4-[(3aS,6aR)-2,3,3a,4,6,6a-hexahydro-1H-pyrrolo[3,4-c]pyrrole-5-carbonyl]piperazine-1-carbonyl]phenyl]-1-[2-(difluoromethyl)-3-fluoro-4-methoxyphenyl]imidazole-4-carboxamide (40.0 mg, 0.06 mmol, 1.0 eq), polyformaldehyde (5.45 mg, 0.18 mmol, 3.0 eq), sodium acetate (14.89 mg, 0.18 mmol, 3.0 eq), and sodium cyanoborohydride (19.01 mg, 0.3 mmol, 5.0 eq) in methanol (3 mL) was stirred at 25° C. for 16 h. The resulting solution was concentrated under reduced pressure. The crude product was purified by Prep-HPLC (chiralpak-AS, CO₂-MEOH(DEtOAc)) to afford 5-amino-N-[3-chloro-4-[4-[(3aS,6aR)-2-methyl-1,3,3a,4,6,6a-hexahydropyrrolo[3,4-c]pyrrole-5-carbonyl]piperazine-1-carbonyl]phenyl]-1-[2-(difluoromethyl)-3-fluoro-4-methoxyphenyl]imidazole-4-carboxamide; 1:1 2,2,2-trifluoroacetic acid (35.5 mg, 0.04 mmol, 73.3% yield). MS [M+H]⁺: 675.3

### Compound 168

### N-[4-[4-[(3aS,6aR)-2-methyl-1,3,3a,4,6,6a-hexahydropyrrolo[3,4-c]pyrrole-5-carbonyl]piperazine-1-carbonyl]-3-fluoro-phenyl]-5-amino-1-[2-(difluoromethyl)-3-fluoro-4-methoxyphenyl]imidazole-4-carboxamide; 1:1 2,2,2-trifluoroacetic acid

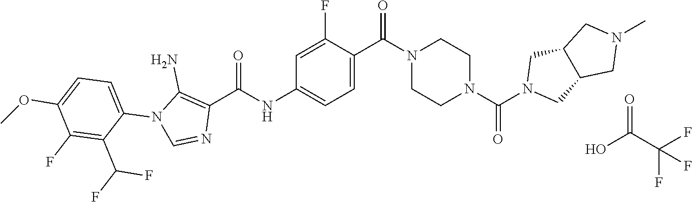

### Step 1: tert-butyl (3aR,6aS)-2-[4-[4-[[5-amino-1-[2-(difluoromethyl)-3-fluoro-4-methoxyphenyl]imidazole-4-carbonyl]amino]-2-fluorobenzoyl]piperazine-1-carbonyl]-1,3,3a,4,6,6a-hexahydropyrrolo[3,4-c]pyrrole-5-carboxylate

To a solution of tert-butyl (3aR,6aS)-2-[4-(4-amino-2-fluorobenzoyl)piperazine-1-carbonyl]-1,3,3a,4,6,6a-hexahydropyrrolo[3,4-c]pyrrole-5-carboxylate (90.96 mg, 0.2 mmol, 0.7 eq) and pyridine (0.46 mL, 5.63 mmol, 20.0 eq) in methylene chloride (2 mL) was added a suspension of 5-amino-1-[2-(difluoromethyl)-3-fluoro-4-methoxyphenyl]imidazole-4-carbonyl chloride (90.0 mg, 0.28 mmol, 1.0 eq) in methylene chloride at 25° C. The mixture was stirred at 25° C. for 1 h under N₂. The resulting solution was evaporated under reduced pressure. The residue was purified by column chromate-graphy (silica gel, 0 to 10% MeOH in methylene chloride) to give tert-butyl (3aR,6aS)-2-[4-[4-[[5-amino-1-[2-(difluoromethyl)-3-fluoro-4-methoxyphenyl]imidazole-4-carbonyl]amino]-2-fluorobenzoyl]piperazine-1-carbonyl]-1,3,3a,4,6,6a-hexahydropyrrolo[3,4-c]pyrrole-5-carboxylate (157.0 mg, 0.21 mmol, 72.6% yield). MS [M+H]⁺: 745.4

### Step 2: N-[4-[4-[(3aS,6aR)-2,3,3a, 4,6,6a-hexahydro-1H-pyrrolo[3,4-c]pyrrole-5-carbonyl]piperazine-1-carbonyl]-3-fluorophenyl]-5-amino-1-[2-(difluoromethyl)-3-fluoro-4-methoxyphenyl]imidazole-4-carboxamide; 1:1 2,2,2-trifluoroacetic acid

A solution of tert-butyl (3aR,6aS)-2-[4-[4-[[5-amino-1-[2-(difluoromethyl)-3-fluoro-4-methoxyphenyl]imidazole-4-carbonyl]amino]-2-fluorobenzoyl]piperazine-1-carbonyl]-1,3,3a,4,6,6a-hexahydropyrrolo[3,4-c]pyrrole-5-carboxylate (147.0 mg, 0.2 mmol, 1.0 eq) and trifluoroacetic acid (1.0 mL) in methylene chloride (1 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40). The product was freeze-dried to afford N-[4-[4-[(3aS,6aR)-2,3,3a,4,6,6a-hexahydro-1H-pyrrolo[3,4-c]pyrrole-5-carbonyl]piperazine-1-carbonyl]-3-fluorophenyl]-5-amino-1-[2-(difluoromethyl)-3-fluoro-4-methoxyphenyl]imidazole-4-carboxamide; 1:1 2,2,2-trifluoroacetic acid (47.4 mg, 0.06 mmol, 31.6% yield). MS [M+H]⁺: 645.0

### Step 3: N-[4-[4-[(3aS,6aR)-2-methyl-1,3,3a,4,6,6a-hexahydropyrrolo[3,4-c]pyrrole-5-carbonyl]piperazine-1-carbonyl]-3-fluorophenyl]-5-amino-1-[2-(difluoromethyl)-3-fluoro-4-methoxyphenyl]imidazole-4-carboxamide; 1:1 2,2,2-trifluoroacetic acid

A solution of N-[4-[4-[(3aS,6aR)-2,3,3a,4,6,6a-hexahydro-1H-pyrrolo[3,4-c]pyrrole-5-carbonyl]piperazine-1-carbonyl]-3-fluorophenyl]-5-amino-1-[2-(difluoromethyl)-3-fluoro-4-methoxyphenyl]imidazole-4-carboxamide (40.0 mg, 0.06 mmol, 1.0 eq), paraformadehyde (61.68 mg, 0.06 mmol, 1.0 eq), NaCNBH₃ (19.55 mg, 0.31 mmol, 5.0 eq) and NaOAc (15.26 mg, 0.19 mmol, 3.0 eq) in methanol (2 mL) was stirred at 25° C. for 2 h. The resulting solution was partitioned between water and ethyl acetate. The organic fraction was concentrated under reduced pressure at 30° C. The resulting solution was concentrated under reduced pressure. The crude product was purified by Prep-HPLC (Gemini C18 150×21.2 mm, 5 μm, MeCN-H₂O (0.1% TFA), 15-40). The product was freeze-dried to afford N-[4-[4-[(3aS,6aR)-2-methyl-1,3,3a,4,6,6a-hexahydropyrrolo[3,4-c]pyrrole-5-carbonyl]piperazine-1-carbonyl]-3-fluorophenyl]-5-amino-1-[2-(difluoromethyl)-3-fluoro-4-methoxyphenyl]imidazole-4-carboxamide; 1:1 2,2,2-trifluoroacetic acid (44.7 mg, 0.06 mmol, 91.6% yield). MS [M+H]⁺: 659.2

### Compound 169

### 5-amino-N-[3-chloro-4-[4-[(1R,5S,6r)-3-(3-hydroxypropyl)-3-azabicyclo[3.1.0]hexane-6-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

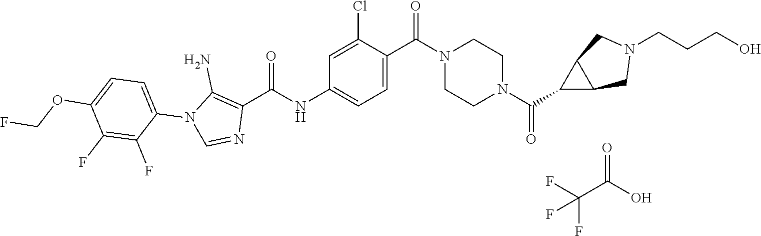

### Compound 170

### 5-amino-N-[3-chloro-4-[4-[(1R,5S,6r)-3-[2-(2-hydroxyethoxy)ethyl]-3-azabicyclo[3.1.0]hexane-6-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

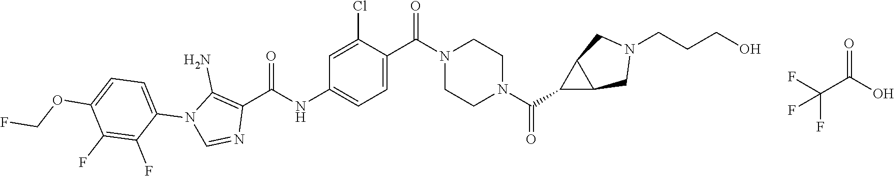 [SMILES: Nc1c(C(=O)Nc2ccc(C(=O)N3CCN(C(=O)[C@H]4[C@@H]5CN(CCCO)C[C@@H]54)CC3)c(Cl)c2)ncn1-c1ccc(OCF)c(F)c1F ; O=C(O)C(F)(F)F]

### Compound 171

### 5-amino-N-[3-chloro-4-[4-[(1R,5S,6r)-3-(2-hydroxyethyl)-3-azabicyclo[3.1.0]hexane-6-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

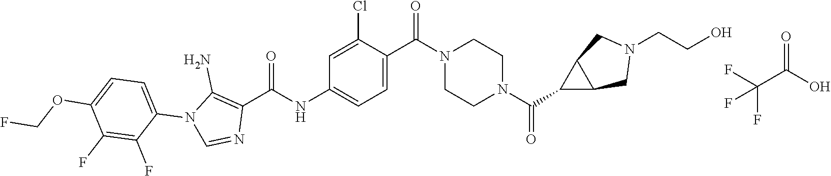

### Step 1: tert-butyl (1R,5S)-6-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate

To a solution of tert-butyl (1R,5S)-6-[4-(4-amino-2-chlorobenzoyl)piperazine-1-carbonyl]-3-azabicyclo[3.1.0]hexane-3-carboxylate (264.39 mg, 0.59 mmol, 1.0 eq) in methylene chloride (5 mL) was added pyridine (0.95 mL, 11.78 mmol, 20.0 eq), 5-amino-1-[2,3-difluoro-4-(fluoromethoxy)phenyl]imidazole-4-carbonyl chloride (180.0 mg, 0.59 mmol, 1.0 eq). The reaction was stirred at 25° C. for 15 h. The reaction was concentrated to dryness and the residue was taken up in methylene chloride (20 mL). The organics were washed with 2×20 mL water followed by 20 mL brine. The organics were then separated and dried (MgSO₄) before concentrating to dryness. The crude was then purified by flash column chromatography eluting with 12% MeOH in methylene chloride to afford tert-butyl (1R,5S)-6-[4-[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (290.0 mg, 0.4 mg, 0.4 mmol, 54.2% yield). MS [M−tBu+H]⁺: 662.2

### Step 2: 5-amino-N-[4-[4-[(1R,5S)-3-azabicyclo[3.1.0]hexane-6-carbonyl]piperazine-1-carbonyl]-3-chloro-phenyl]-1-[2,3-difluoro-4-(fluoromethoxy)phenyl]imidazole-4-carboxamide

To a solution of tert-butyl (1R,5S)-6-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (270.0 mg, 0.38 mmol, 1.0 eq) in methylene chloride (3 mL) was added trifluoroacetic acid (1.0 mL). The reaction was stirred at 25° C. for 1 h. The reaction was concentrated to dryness to afford a crude product 5-amino-N-[4-[4-[(1R,5S)-3-azabicyclo[3.1.0]hexane-6-carbonyl]piperazine-1-carbonyl]-3-chloro-phenyl]-1-[2,3-difluoro-4-(fluoromethoxy)phenyl]imidazole-4-carboxamide (180.0 mg, 0.29 mmol, 51.4% yield). MS [M+H]⁺: 618.0

### Step 3: 5-amino-N-[4-[4-[(1R,5S)-3-[3-[tert-butyl(dimethyl)silyl]oxypropyl]-3-azabicyclo[3.1.0]hexane-6-carbonyl]piperazine-1-carbonyl]-3-chloro-phenyl]-1-[2,3-difluoro-4-(fluoromethoxy)phenyl]imidazole-4-carboxamide

A solution of 5-amino-N-[4-[4-[(1R,5S)-3-azabicyclo[3.1.0]hexane-6-carbonyl]piperazine-1-carbonyl]-3-chloro-phenyl]-1-[2,3-difluoro-4-(fluoromethoxy)phenyl]imidazole-4-carboxamide (50.0 mg, 0.08 mmol, 1.0 eq) in methanol (3 mL) was added sodium acetate (0.02 mL, 0.24 mmol, 3.0 eq), 3-[tert-butyl(dimethyl)silyl]oxypropanal (30.48 mg, 0.16 mmol, 2.0 eq) and sodium cyanoborohydride (25.42 mg, 0.4 mmol, 5.0 eq). The reaction was stirred at 25° C. for 4 h. The reaction was concentrated to dryness and the residue was taken up in methylene chloride (10 mL). The organics were washed with 2×10 mL water followed by 10 mL brine. The organics were then separated and dried (MgSO₄) before concentrating to dryness. The crude was then purified by flash column chromatography eluting with with 10% MeOH in methylene chloride to afford 5-amino-N-[4-[4-[(1R,5S)-3-[3-[tert-butyl(dimethyl)silyl]oxypropyl]-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide (40.0 mg, 0.05 mmol, 57.6% yield). MS [M+H]⁺: 790.2

### Step 4: 5-amino-N-[3-chloro-4-[4-[(1R,5S)-3-(3-hydroxypropyl)-3-azabicyclo[3.1.0]hexane-6-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

To a solution of 5-amino-N-[4-[4-[(1R,5S)-3-[3-[tert-butyl(dimethyl)silyl]oxypropyl]-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide (35.0 mg, 0.04 mmol, 1.0 eq) in THF (3 mL) was added triethylamine trihydrofluoride (21.42 mg, 0.13 mmol, 3.0 eq). The reaction was stirred at 25° C. for 5 h. The reaction was concentrated to dryness to afford a crude product. The crude was purified by Prep-HPLC (Gemini-C18 150×21.2 mm, 5 μm, MeCN-H₂O (0.1% TFA), 20-40) to afford 5-amino-N-[3-chloro-4-[4-[(1R,5S)-3-(3-hydroxypropyl)-3-azabicyclo[3.1.0]hexane-6-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (7.2 mg, 0.01 mmol, 19.7% yield). MS [M+H]⁺: 676.0

### Step 5: 5-amino-N-[4-[4-[(1R,5S)-3-[2-[2-[tert-butyl(diphenyl)silyl]oxyethoxy]ethyl]-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide

To a solution of 5-amino-N-[4-[4-[(1R,5S)-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide (50.0 mg, 0.08 mmol, 1.0 eq) in methanol (3 mL) was added 2-[2-[tert-butyl(diphenyl)silyl]oxyethoxy]acetaldehyde (55.42 mg, 0.16 mmol, 2.0 eq), sodium acetate (0.02 mL, 0.24 mmol, 3.0 eq) and sodium cyanoborohydride (25.42 mg, 0.4 mmol, 5.0 eq). The reaction was stirred at 25° C. for 15 h. The reaction was concentrated to dryness and the residue was taken up in EtOAc (20 mL) and the organics washed with 2×10 mL water followed by 10 mL brine. The organics were then separated and dried (MgSO₄) before concentrating to dryness. The crude was then purified by flash column chromatography eluting with 5% MeOH in methylene chloride to afford 5-amino-N-[4-[4-[(1R,5S)-3-[2-[2-[tert-butyl(diphenyl)silyl]oxyethoxy]ethyl]-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide (47.0 mg, 0.05 mmol, 55.2% yield). MS [M+H]⁺: 943.9

### Step 6: 5-amino-N-[3-chloro-4-[4-[(1R,5S)-3-[2-(2-hydroxyethoxy)ethyl]-3-azabicyclo[3.1.0]hexane-6-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

To a solution of 5-amino-N-[4-[4-[(1R,5S)-3-[2-[2-[tert-butyl(diphenyl)silyl]oxyethoxy]ethyl]-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide (42.0 mg, 0.04 mmol, 1.0 eq) in THF (2 mL) was added triethylamine trihydrofluoride (21.51 mg, 0.13 mmol, 3.0 eq). The reaction was stirred at 25° C. for 3 h. The reaction was concentrated to dryness to afford a crude product. The crude was purified by Prep-HPLC (Gemini-C18 150×21.2 mm, 5 μm, MeCN-H₂O (0.1% TFA), 15-50) to afford 5-amino-N-[3-chloro-4-[4-[(1R,5S)-3-[2-(2-hydroxyethoxy)ethyl]-3-azabicyclo[3.1.0]hexane-6-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (22.3 mg, 0.03 mmol, 71.0% yield). MS [M+H]⁺: 706.0

### Step 7: 5-amino-N-[4-[4-[(1R,5S)-3-[2-[tert-butyl(dimethyl)silyl]oxyethyl]-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide

To a solution of 5-amino-N-[4-[4-[(1R,5S)-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide (50.0 mg, 0.08 mmol, 1.0 eq) in methanol (3 mL) was added sodium acetate (0.02 mL, 0.24 mmol, 3.0 eq), (tert-butyldimethylsilyloxy) acetaldehyde (28.21 mg, 0.16 mmol, 2.0 eq) and sodium cyanoborohydride (25.42 mg, 0.4 mmol, 5.0 eq). The reaction was stirred at 25° C. for 4 h. The reaction was concentrated to dryness and the residue was taken up in methylene chloride (20 mL). The organics were washed with 2×20 mL water followed by 20 mL brine. The organics were then separated and dried (MgSO₄) before concentrating to dryness. The crude was then purified by flash column chromatography eluting with with 12% MeOH in methylene chloride to afford 5-amino-N-[4-[4-[(1R,5S)-3-[2-[tert-butyl(dimethyl)silyl]oxyethyl]-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide (50.0 mg, 0.06 mmol, 72.8% yield). MS [M+H]⁺: 776.2

### Step 8: 5-amino-N-[3-chloro-4-[4-[(1R,5S)-3-(2-hydroxyethyl)-3-azabicyclo[3.1.0]hexane-6-carbonyl]piperazine-1-carbonyl]phenyl]-1-[2,3-difluoro-4-(fluoromethoxy)phenyl]imidazole-4-carboxamide; 1:1 2,2,2-trifluoroacetic acid

To a solution of 5-amino-N-[4-[4-[(1R,5S)-3-[2-[tert-butyl(dimethyl)silyl]oxyethyl]-3-azabicyclo[3.1.0]hexane-6-carbonyl]piperazine-1-carbonyl]-3-chlorophenyl]-1-[2,3-difluoro-4-(fluoromethoxy)phenyl]imidazole-4-carboxamide (45.0 mg, 0.06 mmol, 1.0 eq) in THF (3 mL) was added triethylamine trihydrofluoride (28.03 mg, 0.17 mmol, 3.0 eq). The reaction was stirred at 25° C. for 5 h. The reaction was concentrated to dryness to afford a crude product. The crude product was purified by Prep-HPLC (Gemini-C18 150×21.2 mm, 5 μm, MeCN-H₂O (0.1% TFA), 15-50) to afford 5-amino-N-[3-chloro-4-[4-[(1R,5S)-3-(2-hydroxyethyl)-3-azabicyclo[3.1.0]hexane-6-carbonyl]piperazine-1-carbonyl]phenyl]-1-[2,3-difluoro-4-(fluoromethoxy)phenyl]imidazole-4-carboxamide; 1:1 2,2,2-trifluoroacetic acid (32.0 mg, 0.04 mmol, 79.9% yield). MS [M+H]⁺: 662.0

### Compound 172

### N-[4-[4-[(3aR,6aR)-2,3,3a,4,6,6a-hexahydro-1H-pyrrolo[3,4-c]pyrrole-5-carbonyl]piperazine-1-carbonyl]-3-chloro-phenyl]-5-amino-1-[2,3-difluoro-4-(fluoromethoxy)phenyl]imidazole-4-carboxamide; 1:1 2,2,2-trifluoroacetic acid

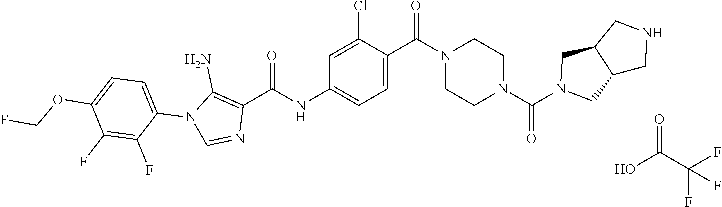 [SMILES: Nc1c(C(=O)Nc2ccc(C(=O)N3CCN(C(=O)N4C[C@H]5CNC[C@@H]5C4)CC3)c(Cl)c2)ncn1-c1ccc(OCF)c(F)c1F ; O=C(O)C(F)(F)F]

### Step 1: tert-butyl (3aR,6aR)-2-[4-(2-chloro-4-nitrobenzoyl)piperazine-1-carbonyl]-1,3,3a,4,6,6a-hexahydropyrrolo[3,4-c]pyrrole-5-carboxylate

A solution of (2-chloro-4-nitrophenyl)-piperazin-1-yl-methanone (698.69 mg, 2.59 mmol, 1.1 eq) and triphosgene (349.46 mg, 1.18 mmol, 0.5 eq) in methylene chloride (5 mL) was stirred at 25° C. for 0.5 h. tert-Butyl (3aR,6aR)-2,3,3a,4,6,6a-hexahydro-1H-pyrrolo[3,4-c]pyrrole-5-carboxylate (500.0 mg, 2.36 mmol, 1.0 eq) and triethylamine (3.28 mL, 23.55 mmol, 10.0 eq) were then added while stirring at 25° C. After 1 h the resulting solution was partitioned between water and ethyl acetate. After concentration of the organic fraction, the crude was purified by flash column chromatography eluting with 87:13 methylene chloride:MeOH to give tert-butyl (3aR,6aR)-2-[4-(2-chloro-4-nitrobenzoyl)piperazine-1-carbonyl]-1,3,3a, 4,6,6a-hexahydropyrrolo[3,4-c]pyrrole-5-carboxylate (620.0 mg, 1.22 mmol, 27.0% yield). MS [M+H]⁺: 508.1.

### Step 2: tert-butyl (3aR,6aR)-2-[4-(4-amino-2-chlorobenzoyl)piperazine-1-carbonyl]-1,3,3a,4,6,6a-hexahydropyrrolo[3,4-c]pyrrole-5-carboxylate

A solution of tert-butyl (3aR,6aR)-2-[4-(2-chloro-4-nitrobenzoyl)piperazine-1-carbon-yl]-1,3,3a,4,6,6a-hexahydropyrrolo[3,4-c]pyrrole-5-carboxylate (570.0 mg, 1.12 mmol, 1.0 eq), ammonium chloride (300.11 mg, 5.61 mmol, 5.0 eq) and iron (313.32 mg, 5.61 mmol, 5.0 eq) in ethanol (5 mL) and water (2 mL) was stirred at 50° C. for 2 h. After removal of the iron by filtration, the resulting solution was concentrated under reduced pressure, diluted with EtOAc (30 mL), washed with H₂O (3×20 mL), dried over Na₂SO₄ and concentrated under reduced pressure. The residue was then purified by flash column chromatography eluting with 9:1 methylene chloride:MeOH to give tert-butyl (3aR,6aR)-2-[4-(4-amino-2-chlorobenzoyl)piperazine-1-carbonyl]-1,3,3a,4,6,6a-hexahydropyrrolo[3,4-c]pyrrole-5-carboxylate (248.0 mg, 0.52 mmol, 44.8% yield). MS [M+H]⁺: 478.3.

### Step 3: tert-butyl (3aR,6aR)-2-[4-[4-[[5-amino-1-[2,3-difluoro-4-(fluoromethoxy)phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate

To a solution of tert-butyl (3aR,6aR)-2-[4-(4-amino-2-chlorobenzoyl)piperazine-1-carbonyl]-1,3,3a,4,6,6a-hexahydropyrrolo[3,4-c]pyrrole-5-carboxylate (54.74 mg, 0.11 mmol, 0.7 eq) and pyridine (0.26 mL, 3.27 mmol, 20.0 eq) was added a suspension of 5-amino-1-[2,3-difluoro-4-(fluoromethoxy)phenyl]imidazole-4-carbonyl chloride (50.0 mg, 0.16 mmol, 1.0 eq) in methylene chloride (1 mL) at 25° C. The mixture was stirred at 25° C. for 1 h under N₂. The resulting solution was evaporated under reduced pressure. The residue was purified by column chromatography (silica gel, 0 to 10% MeOH in methylene chloride) to give tert-butyl (3aR,6aR)-2-[4-[4-[[5-amino-1-[2,3-difluoro-4-(fluoromethoxy)phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (78.0 mg, 0.1 mmol, 61.3% yield). MS [M+H]⁺: 746.9.

### Step 4: N-[4-[4-[(3aR,6aR)-2,3,3a,4,6,6a-hexahydro-1H-pyrrolo[3,4-c]pyrrole-5-carbonyl]piperazine-1-carbonyl]-3-chloro-phenyl]-5-amino-1-[2,3-difluoro-4-(fluoromethoxy)phenyl]imidazole-4-carboxamide; 1:1 2,2,2-trifluoroacetic acid

A solution of tert-butyl (3aR,6aR)-2-[4-[4-[[5-amino-1-[2,3-difluoro-4-(fluoromethoxy)phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (68.0 mg, 0.09 mmol, 1.0 eq) and trifluoroacetic acid (1.0 mL) in methylene chloride (1 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40). The product was freeze-dried to afford N-[4-[4-[(3aR,6aR)-2,3,3a,4,6,6a-hexahydro-1H-pyrrolo[3,4-c]pyrrole-5-carbonyl]piperazine-1-carbonyl]-3-chlorophenyl]-5-amino-1-[2,3-difluoro-4-(fluoromethoxy)phenyl]imidazole-4-carboxamide; 1:1 2,2,2-trifluoroacetic acid (42.6 mg, 0.06 mmol, 71.0% yield). MS [M+H]⁺: 646.9.

### Compound 173

### N-[4-[4-[(3aR,6aR)-2-methyl-1,3,3a,4,6,6a-hexahydropyrrolo[3,4-c]pyrrole-5-carbonyl]piperazine-1-carbonyl]-3-chlorophenyl]-5-amino-1-[2-(difluoromethyl)-3-fluoro-4-(fluoromethoxy)phenyl]imidazole-4-carboxamide; 1:1 2,2,2-trifluoroacetic acid

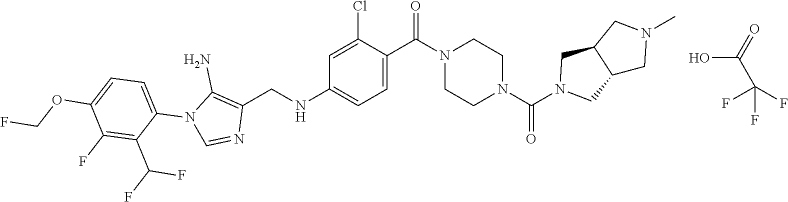 [SMILES: CN1C[C@@H]2CN(C(=O)N3CCN(C(=O)c4ccc(NCc5ncn(-c6ccc(OCF)c(F)c6C(F)F)c5N)cc4Cl)CC3)C[C@H]2C1 ; O=C(O)C(F)(F)F]

### Compound 174

### N-[4-[4-[(3aR,6aR)-2,3,3a,4,6,6a-hexahydro-1H-pyrrolo[3,4-c]pyrrole-5-carbonyl]piperazine-1-carbonyl]-3-chlorophenyl]-5-amino-1-[2-(difluoromethyl)-3-fluoro-4-(fluoromethoxy)phenyl]imidazole-4-carboxamide; 1:1 2,2,2-trifluoroacetic acid

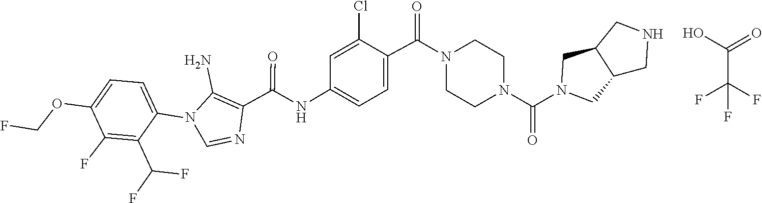 [SMILES: Nc1c(C(=O)Nc2ccc(C(=O)N3CCN(C(=O)N4C[C@H]5CNC[C@@H]5C4)CC3)c(Cl)c2)ncn1-c1ccc(OCF)c(F)c1C(F)F ; O=C(O)C(F)(F)F]

### Step 1: 5-amino-1-[2-(difluoromethyl)-3-fluoro-4-(fluoromethoxy)phenyl]imidazole-4-carboxylic acid

Sodium hydroxide (575.9 mg, 14.4 mmol, 5.0 eq) was added to a solution of ethyl 5-amino-1-[2-(difluoromethyl)-3-fluoro-4-(fluoromethoxy)phenyl]imidazole-4-carboxylate (1000.0 mg, 2.88 mmol, 1.0 eq) in isopropyl alcohol (10 mL) and water (5 mL). The resulting mixture was stirred at 50° C. for 2 h. The mixture was adjusted to pH 5-6 with 2 N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure give 5-amino-1-[2-(difluoromethyl)-3-fluoro-4-(fluoromethoxy)phenyl]imidazole-4-carboxylic acid (400.0 mg, 1.25 mmol, 43.3% yield). MS [M+H]⁺: 320.2.

### Step 2: 5-amino-1-[2-(difluoromethyl)-3-fluoro-4-(fluoromethoxy)phenyl]imidazole-4-carbonyl chloride

A solution of 5-amino-1-[2-(difluoromethyl)-3-fluoro-4-(fluoromethoxy)phenyl]imidazole-4-carboxylic acid (100.0 mg, 0.31 mmol, 1.0 eq) in thionyl chloride (372.7 mg, 3.13 mmol, 10.0 eq) was stirred at 25° C. for 1 h. The resulting solution was concentrated under reduced pressure to give 5-amino-1-[2-(difluoromethyl)-3-fluoro-4-(fluoromethoxy)phenyl]imidazole-4-carbonyl chloride (100.0 mg, 0.3 mmol, 69.8% yield). MS [(M−Cl+OCH₃)+H]⁺: 334.0

### Step 3: tert-butyl (3aR,6aR)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate

To a solution of tert-butyl (3aR,6aR)-2-[4-(4-amino-2-chlorobenzoyl)piperazine-1-carbonyl]-1,3,3a,4,6,6a-hexahydropyrrolo[3,4-c]pyrrole-5-carboxylate (141.56 mg, 0.3 mmol, 1.0 eq) and pyridine (0.24 mL, 2.96 mmol, 10.0 eq) in methylene chloride (2 mL) were added 5-amino-1-[2-(difluoromethyl)-3-fluoro-4-(fluoromethoxy)phenyl]imidazole-4-carbonyl chloride (100.0 mg, 0.3 mmol, 1.0 eq). The resulting mixture was stirred at 25° C. for 3 h. The reaction was concentrated to dryness and the residue was taken up in EtOAc (50 ml). The organics washed with 2×10 mL water followed by 20 mL brine. The organics were then separated and dried (Na₂SO₄) before concentrating to dryness. The crude was then purified by flash column chromatography eluting with 5% MeOH in methylene chloride to give tert-butyl (3aR,6aR)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (140.0 mg, 0.18 mmol, 57.7% yield). MS [M+H]⁺: 778.9

### Step 4: N-[4-[4-[(3aR,6aR)-2,3,3a,4,6,6a-hexahydro-1H-pyrrolo[3,4-c]pyrrole-5-carbonyl]piperazine-1-carbonyl]-3-chloro-phenyl]-5-amino-1-[2-(difluoromethyl)-3-fluoro-4-(fluoromethoxy)phenyl]imidazole-4-carboxamide; 1:1 2,2,2-trifluoroacetic acid

A solution of tert-butyl (3aR,6aR)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-1,3,3a,4,6,6a-hexahydropyrrolo[3,4-c]pyrrole-5-carboxylate (130.0 mg, 0.17 mmol, 1.0 eq) and trifluoroacetic acid (1.0 mL, 13 mmol, 78 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA), 25-40) to give N-[4-[4-[(3aR,6aR)-2,3,3a,4,6,6a-hexahydro-1H-pyrrolo[3,4-c]pyrrole-5-carbonyl]piperazine-1-carbonyl]-3-chlorophenyl]-5-amino-1-[2-(difluoromethyl)-3-fluoro-4-(fluoromethoxy)phenyl]imidazole-4-carboxamide; 1:1 2,2,2-trifluoroacetic acid (80.0 mg, 0.1 mmol, 60.5% yield). MS [M+H]⁺: 679.3

### Step 5: N-[4-[4-[(3aR,6aR)-2-methyl-1,3,3a,4,6,6a-hexahydropyrrolo[3,4-c]pyrrole-5-carbonyl]piperazine-1-carbonyl]-3-chlorophenyl]-5-amino-1-[2-(difluoromethyl)-3-fluoro-4-(fluoromethoxy)phenyl]imidazole-4-carboxamide; 1:1 2,2,2-trifluoroacetic acid

A solution of N-[4-[4-[(3aR,6aR)-2,3,3a,4,6,6a-hexahydro-1H-pyrrolo[3,4-c]pyrrole-5-carbonyl]piperazine-1-carbonyl]-3-chlorophenyl]-5-amino-1-[2-(difluoromethyl)-3-fluoro-4-(fluoromethoxy)phenyl]imidazole-4-carboxamide (40.0 mg, 0.06 mmol, 1.0 eq), polyformaldehyde (5.31 mg, 0.18 mmol, 3.0 eq), sodium acetate (14.5 mg, 0.18 mmol, 3.0 eq), and sodium cyanoborohydride (18.51 mg, 0.29 mmol, 5.0 eq) in methanol (3 mL) was stirred at 25° C. for 16 h. The resulting solution was concentrated under reduced pressure. The crude product was purified by Prep-HPLC (chiral Pak-AS, CO₂-MeOH(EtOAc)) to afford N-[4-[4-[(3aR,6aR)-2-methyl-1,3,3a,4,6,6a-hexahydropyrrolo[3,4-c]pyrrole-5-carbonyl]piperazine-1-carbonyl]-3-chlorophenyl]-5-amino-1-[2-(difluoromethyl)-3-fluoro-4-(fluoromethoxy)phenyl]imidazole-4-carboxamide; 1:1 2,2,2-trifluoroacetic acid (6.0 mg, 0.01 mmol, 12.0% yield). MS [M+H]⁺: 693.0

### Compound 175

### N-[4-[(3aS,6aR)-5-[(1S,5R,6r)-3-methyl-3-azabicyclo[3.1.0]hexane-6-carbonyl]-1,3,3a,4,6,6a-hexahydropyrrolo[3,4-c]pyrrole-2-carbonyl]-3-chlorophenyl]-5-amino-1-[2,3-difluoro-4-(fluoromethoxy)phenyl]imidazole-4-carboxamide; 1:1 2,2,2-trifluoroacetic acid

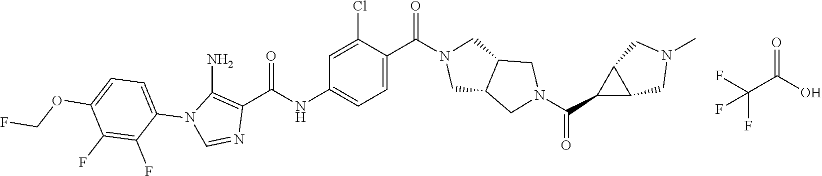

### Step 1: tert-butyl (3aR,6aS)-2-(2-chloro4-nitrobenzoyl)-1,3,3a,4,6,6a-hexahydropyrrolo[3,4-c]pyrrole-5-carboxylate

A solution of 2-chloro-4-nitrobenzoic acid (2.0 g, 9.92 mmol, 1.0 eq), 1,1-dimethylethyl hexahydropyrrolo[3,4-c]pyrrole-2 (1H)-carbo (2.11 g, 9.92 mmol, 1.0 eq), 2-(7-azabenzotriazol-1-yl)-N,N,N′,N′-tetramethyluronium hexafluorophosphate (7.54 g, 19.85 mmol, 2.0 eq) and N,N-diisopropylethylamine (5.18 mL, 29.77 mmol, 3.0 eq) in methylene chloride (20 mL) was stirred at 20° C. for 1 h. The resulting solution was concentrated under reduced pressure. The residue was applied on a silica gel column and eluted with 3:1 ethyl acetate:isohexane to afford tert-butyl (3aR,6aS)-2-(2-chloro-4-nitrobenzoyl)-1,3,3a,4,6,6a-hexahydropyrrolo[3,4-c]pyrrole-5-carboxylate (3.8 g, 9.6 mmol, 96.8% yield). MS [M−tBu+H]⁺: 340

### Step 2: [(3aS,6aR)-2,3,3a,4,6,6a-hexahydro-1H-pyrrolo[3,4-c]pyrrol-5-yl]-(2-chloro-4-nitrophenyl) methanone

A solution of tert-butyl (3aR,6aS)-2-(2-chloro-4-nitrobenzoyl)-1,3,3a,4,6,6a-hexahydropyrrolo[3,4-c]pyrrole-5-carboxylate (3.0 g, 7.58 mmol, 1.0 eq) and hydrogen chloride (8 mL, 1M in 1,4-dioxane) in 1,4-dioxane (20 mL) was stirred at 20° C. for 2 h. The resulting solution was concentrated under reduced pressure to afford [(3aS,6aR)-2,3,3a,4,6,6a-hexahydro-1H-pyrrolo[3,4-c]pyrrol-5-yl]-(2-chloro-4-nitrophenyl) methanone (2.0 g, 6.76 mmol, 89.2% yield). MS [M+H]⁺: 296

### Step 3: tert-butyl (1S,5R)-6-[(3aS,6aR)-2-(2-chloro-4-nitrobenzoyl)-1,3,3a,4,6,6a-hexahydropyrrolo[3,4-c]pyrrole-5-carbonyl]-3-azabicyclo[3.1.0]hexane-3-carboxylate

A solution of [(3aS,6aR)-2,3,3a, 4,6,6a-hexahydro-1H-pyrrolo[3,4-c]pyrrol-5-yl]-(2-chloro-4-nitrophenyl) methanone (500.0 mg, 1.69 mmol, 1.0 eq), (1S,5R)-3-tert-butoxycarbonyl-3-azabicyclo[3.1.0]hexane-6-carboxylic acid (384.25 mg, 1.69 mmol, 1.0 eq), 2-(7-azabenzotriazol-1-yl)-N,N,N′,N′-tetramethyluronium hexafluorophosphate (1.28 g, 3.38 mmol, 2.0 eq) and 2-(7-azabenzotriazol-1-yl)-N,N,N′,N′-tetramethyluronium hexafluorophosphate (1.28 g, 3.38 mmol, 2.0 eq) in methylene chloride (20 mL) was stirred at 20° C. for 1 h. The resulting solution was concentrated under reduced pressure. The residue was applied on a silica gel column and eluted with 4:1 ethyl acetate:isohexane to afford tert-butyl (1S,5R)-6-[(3aS,6aR)-2-(2-chloro-4-nitrobenzoyl)-1,3,3a,4,6,6a-hexahydropyrrolo[3,4-c]pyrrole-5-carbonyl]-3-azabicyclo[3.1.0]hexane-3-carboxylate (780.0 mg, 1.54 mmol, 91.4% yield). MS [M−tBu+H]⁺: 449

### Step 4: tert-butyl (1S,5R)-6-[(3aS,6aR)-2-(4-amino-2-chlorobenzoyl)-1,3,3a,4,6,6a-hexahydropyrrolo[3,4-c]pyrrole-5-carbonyl]-3-azabicyclo[3.1.0]hexane-3-carboxylate

To a solution of tert-butyl (1S,5R)-6-[(3aS,6aR)-2-(2-chloro-4-nitrobenzoyl) 1,3,3a,4,6,6a-hexahydropyrrolo[3,4-c]pyrrole-5-carbonyl]-3-azabicyclo[3.1.0]hexane-3-carboxylate (500.0 mg, 0.99 mmol, 1.0 eq) in ethanol (10 mL) were added iron powder (442.37 mg, 7.92 mmol, 8.0 eq), ammonium chloride (423.72 mg, 7.92 mmol, 8.0 eq) and water (3 mL) under N₂ atmosphere. The resulting mixture was stirred at 60° C. for 2 h. The solid was filtered off. The filtrate was diluted with EtOAc (50 mL), washed with water (3×25 mL), dried over Na₂SO₄ and evaporated under reduced pressure to afford tert-butyl (1S,5R)-6-[(3aS,6aR)-2-(4-amino-2-chlorobenzoyl)-1,3,3a,4,6,6a-hexahydropyrrolo[3,4-c]pyrrole-5-carbonyl]-3-azabicyclo[3.1.0]hexane-3-carboxylate (400.0 mg, 0.84 mmol, 85.0% yield). MS [M−tBu+H]⁺: 419

### Step 5: tert-butyl (1S,5R)-6-[(3aS,6aR)-2-[4-[[5-amino-1-[2,3-difluoro-4-(fluoromethoxy)phenyl]imidazole-4-carbonyl]amino]-2-chlorobenzoyl]-1,3,3a,4,6,6a-hexahydropyrrolo[3,4-c]pyrrole-5-carbonyl]-3-azabicyclo[3.1.0]hexane-3-carboxylate

To a solution of tert-butyl (1S,5R)-6-[(3aS,6aR)-2-(4-amino-2-chlorobenzoyl)-1,3,3a,4,6,6a-hexahydropyrrolo[3,4-c]pyrrole-5-carbonyl]-3-azabicyclo[3.1.0]hexane-3-carboxylate (200.0 mg, 0.42 mmol, 1.0 eq) and pyridine (0.34 mL, 4.21 mmol, 10.0 eq) in methylene chloride (6 mL) was added a suspension of 5-amino-1-[2,3-difluoro-4-(fluoromethoxy)phenyl]imidazole-4-carbonyl chloride (128.7 mg, 0.42 mmol, 1.0 eq) in methylene chloride (6 mL) at 20° C., The mixture was stirred at 20° C. for 3 h under N₂. The resulting solution was evaporated under reduced pressure. The residue was purified by column chromatography (silica gel, 0 to 10% MeOH in methylene chloride) to afford tert-butyl (1S,5R)-6-[(3aS,6aR)-2-[4-[[5-amino-1-[2,3-difluoro-4-(fluoromethoxy)phenyl]imidazole-4-carbonyl]amino]-2-chlorobenzoyl]-1,3,3a,4,6,6a-hexahydropyrrolo[3,4-c]pyrrole-5-carbonyl]-3-azabicyclo[3.1.0]hexane-3-carboxylate (190.0 mg, 0.26 mmol, 60.6% yield). MS [M−tBu+H]⁺: 687.9

### Step 6: N-[4-[(3aS,6aR)-5-[(1S,5R)-3-azoniabicyclo[3.1.0]hexane-6-carbonyl]-1,3,3a,4,6,6a-hexahydropyrrolo[3,4-c]pyrrole-2-carbonyl]-3-chlorophenyl]-5-amino-1-[2,3-difluoro-4-(fluoromethoxy)phenyl]imidazole-4-carboxamide; 1:1 2,2,2-trifluoroacetate

A solution of tert-butyl (1S,5R)-6-[(3aS,6aR)-2-[4-[[5-amino-1-[2,3-difluoro-4-(fluoromethoxy)phenyl]imidazole-4-carbonyl]amino]-2-chlorobenzoyl]-1,3,3a,4,6,6a-hexahydropyrrolo[3,4-c]pyrrole-5-carbonyl]-3-azabicyclo[3.1.0]hexane-3-carboxylate (60.0 mg, 0.08 mmol, 1.0 eq) and trifluoroacetic acid (2.0 mL, 25.96 mmol, 321.97 eq) in methylene chloride (4 mL) was stirred at 20° C. for 1 h. The resulting solution was concentrated under reduced pressure. The residue was purified by Prep-HPLC (Gemini-C18 150×19 mm, 5 μm, MeCN-H₂O (0.1% TFA), 35-45) to afford N-[4-[(3aS,6aR)-5-[(1S,5R)-3-azoniabicyclo[3.1.0]hexane-6-carbonyl]-1,3,3a,4,6,6a-hexahydropyrrolo[3,4-c]pyrrole-2-carbonyl]-3-chlorophenyl]-5-amino-1-[2,3-difluoro-4-(fluoromethoxy)phenyl]imidazole-4-carboxamide; 1:1 2,2,2-trifluoroacetate (43.4 mg, 0.06 mmol, 81.6% yield). MS [M+H]⁺: 644

### Step 7: N-[4-[(3aS,6aR)-5-[(1S,5R)-3-methyl-3-azabicyclo[3.1.0]hexane-6-carbonyl]-1,3,3a,4,6,6a-hexahydropyrrolo[3,4-c]pyrrole-2-carbonyl]-3-chlorophenyl]-5-amino-1-[2,3-difluoro-4-(fluoromethoxy)phenyl]imidazole-4-carboxamide; 1:1 2,2,2-trifluoroacetic acid

A solution of N-[4-[(3aS,6aR)-5-[(1S,5R)-3-azabicyclo[3.1.0]hexane-6-carbonyl]-1,3,3a,4,6,6a-hexahydropyrrolo[3,4-c]pyrrole-2-carbonyl]-3-chlorophenyl]-5-amino-1-[2,3-difluoro-4-(fluoromethoxy)phenyl]imidazole-4-carboxamide (58.0 mg, 0.09 mmol, 1.0 eq) and trifluoroacetic acid (1.0 mL, 12.98 mmol, 144.13 eq) in methylene chloride (2 mL) was stirred at 20° C. for 2 h. The resulting solution was concentrated under reduced pressure. The residue was purified by Prep-HPLC (Gemini-C18 150×19 mm, 5 μm, MeCN-H₂O (0.1% TFA), 35-45) to afford N-[4-[(3aS,6aR)-5-[(1S,5R)-3-methyl-3-azabicyclo[3.1.0]hexane-6-carbonyl]-1,3,3a,4,6,6a-hexahydropyrrolo[3,4-c]pyrrole-2-carbonyl]-3-chlorophenyl]-5-amino-1-[2,3-difluoro-4-(fluoromethoxy)phenyl]imidazole-4-carboxamide; 1:1 2,2,2-trifluoroacetic acid (42.0 mg, 0.05 mmol, 60.1% yield). MS [M+H]⁺: 658

### Compound 176

### 5-amino-N-[3-chloro-4-[4-[(3aS,6aR)-2-methyl-1,3,3a,4,6,6a-hexahydropyrrolo[3,4-c]pyrrole-5-carbonyl]piperidine-1-carbonyl]phenyl]-1-[2-(difluoromethyl)-3-fluoro-4-(fluoromethoxy)phenyl]imidazole-4-carboxamide; 1:1 2,2,2-trifluoroacetic acid

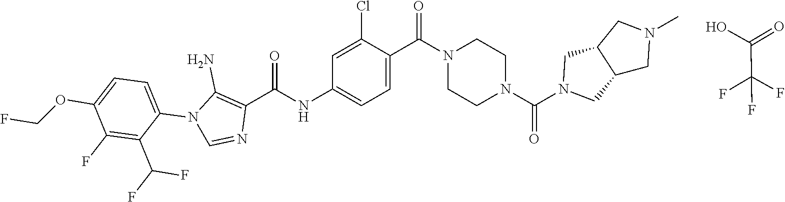

### Step 1: tert-butyl (3aR,6aS)-2-[1-(2-chloro-4-nitrobenzoyl)piperidine-4-carbonyl]-1,3,3a,4,6,6a-hexahydropyrrolo[3,4-c]pyrrole-5-carboxylate

To a solution of 1-(2-chloro-4-nitrobenzoyl)piperidine-4-carboxylic acid (2500.0 mg, 7.99 mmol, 1.0 eq) and 1,1-dimethylethyl hexahydropyrrolo[3,4-c]pyrrole-2 (1H)-carbo (1697.18 mg, 7.99 mmol, 1.0 eq) in methylene chloride (25 mL) was added 2-(7-azabenzotriazol-1-yl)-N,N,N′,N′-tetramethyluronium hexafluorophosphate (6079.75 mg, 15.99 mmol, 2.0 eq) and N,N-diisopropylethylamine (4.18 mL, 23.98 mmol, 3.0 eq). The solution was stirred at 25° C. for 1 h. The reaction was quenched with water (25 mL). The resulting solution was extracted with ethyl acetate (3×20 mL). The combined organic phases were dried over anhydrous sodium sulfate and concentrated under reduced pressure. The residue was purified by flash column to give tert-butyl (3aR,6aS)-2-[1-(2-chloro-4-nitrobenzoyl)piperidine-4-carbonyl]-1,3,3a,4,6,6a-hexahydropyrrolo[3,4-c]pyrrole-5-carboxylate (3300.0 mg, 6.51 mmol, 81.4% yield). MS [(M−Boc)+H]⁺: 407.0

### Step 2: tert-butyl (3aR,6aS)-2-[1-(4-amino-2-chlorobenzoyl)piperidine-4-carbonyl]-1,3,3a,4,6,6a-hexahydropyrrolo[3,4-c]pyrrole-5-carboxylate

A solution of tert-butyl (3aR,6aS)-2-[1-(2-chloro-4-nitrobenzoyl)piperidine-4-carbonyl]-1,3,3a,4,6,6a-hexahydropyrrolo[3,4-c]pyrrole-5-carboxylate (4000.0 mg, 7.89 mmol, 1.0 eq), iron (2203.05 mg, 39.45 mmol, 5.0 eq) and ammonium chloride (2110.14 mg, 39.45 mmol, 5.0 eq) in ethanol (30 mL) and water (6 mL) was stirred at 50° C. for 2 h. The solid was filtered off. The crude was then purified by flash column chromatography eluting with 40% EtOAc in petroleum ether to give the tert-butyl (3aR,6aS)-2-[1-(4-amino-2-chlorobenzoyl)piperidine-4-carbonyl]-1,3,3a,4,6,6a-hexahydropyrrolo[3,4-c]pyrrole-5-carboxylate (3000.0 mg, 6.29 mmol, 79.7% yield). MS [M+H]⁺: 477.1

### Step 3: 5-amino-1-[2-(difluoromethyl)-3-fluoro-4-(fluoromethoxy)phenyl]imidazole-4-carbonyl chloride

A solution of 5-amino-1-[2-(difluoromethyl)-3-fluoro-4-(fluoromethoxy)phenyl]imidazole-4-carboxylic acid (200.0 mg, 0.63 mmol, 1.0 eq) in thionyl chloride (745.4 mg, 6.27 mmol, 10.0 eq) was stirred at 25° C. for 1 h. The resulting solution was concentrated under reduced pressure to give 5-amino-1-[2-(difluoromethyl)-3-fluoro-4-(fluoromethoxy)phenyl]imidazole-4-carbonyl chloride (200.0 mg, 0.59 mmol, 86.2% yield). MS [(M−Cl+OCH₃)+H]⁺: 334.2.

### Step 4: tert-butyl (3aR,6aS)-2-[1-[4-[[5-amino-1-[2-(difluoromethyl)-3-fluoro-4-(fluoromethoxy)phenyl]imidazole-4-carbonyl]amino]-2-chlorobenzoyl]piperidine-4-carbonyl]-1,3,3a,4,6,6a-hexahydropyrrolo[3,4-c]pyrrole-5-carboxylate

To a solution of tert-butyl (3aR,6aS)-2-[1-(4-amino-2-chlorobenzoyl)piperidine-4-carbonyl]-1,3,3a,4,6,6a-hexahydropyrrolo[3,4-c]pyrrole-5-carboxylate (282.53 mg, 0.59 mmol, 1.0 eq) and pyridine (0.48 mL, 5.92 mmol, 10.0 eq) in methylene chloride (2 mL) were added 5-amino-1-[2-(difluoromethyl)-3-fluoro-4-(fluoromethoxy)phenyl]imidazole-4-carbonyl chloride (200.0 mg, 0.59 mmol, 1.0 eq). The resulting mixture was stirred at 25° C. for 1 h. The reaction was concentrated to dryness. The residue was taken up in methylene chloride (20 mL) and the organics washed with 2×5 mL water followed by 5 mL brine. The organics were then separated and dried (Na₂SO₄) before concentrating to dryness. The crude was then purified by flash column chromatography eluting with 4% MeOH in methylene chloride to give tert-butyl (3aR,6aS)-2-[1-[4-[[5-amino-1-[2-(difluoromethyl)-3-fluoro-4-(fluoromethoxy)phenyl]imidazole-4-carbonyl]amino]-2-chlorobenzoyl]piperidine-4-carbonyl]-1,3,3a,4,6,6a-hexahydropyrrolo[3,4-c]pyrrole-5-carboxylate (240.0 mg, 0.31 mmol, 45.8% yield). MS [M+H]⁺: 778.0

### Step 5: 5-amino-N-[3-chloro-4-[4-[(3aS,6aR)-2,3,3a,4,6,6a-hexahydro-1H-pyrrolo[3,4-c]pyrrole-5-carbonyl]piperidine-1-carbonyl]phenyl]-1-[2-(difluoromethyl)-3-fluoro-4-(fluoromethoxy)phenyl]imidazole-4-carboxamide

To a solution of tert-butyl (3aR,6aS)-2-[1-[4-[[5-amino-1-[2-(difluoromethyl)-3-fluoro-4-(fluoromethoxy)phenyl]imidazole-4-carbonyl]amino]-2-chlorobenzoyl]piperidine-4-carbonyl]-1,3,3a,4,6,6a-hexahydropyrrolo[3,4-c]pyrrole-5-carboxylate (240.0 mg, 0.31 mmol, 1.0 eq) and trifluoroacetic acid (1.0 mL, 13 mmol, 42 eq) in methylene chloride (3 mL) was stirred at 25° C. for 1 h. The resulting solution was concentrated under reduced pressure to give 5-amino-N-[3-chloro-4-[4-[(3aS,6aR)-2,3,3a,4,6,6a-hexahydro-1H-pyrrolo[3,4-c]pyrrole-5-carbonyl]piperidine-1-carbonyl]phenyl]-1-[2-(difluoromethyl)-3-fluoro-4-(fluoromethoxy)phenyl]imidazole-4-carboxamide;2,2,2-trifluoroacetic acid (150.0 mg, 0.19 mmol, 60.0% yield). MS [M+H]⁺: 678.3.

### Step 4: 5-amino-N-[3-chloro-4-[4-[(3aS,6aR)-2-methyl-1,3,3a,4,6,6a-hexahydropyrrolo[3,4-c]pyrrole-5-carbonyl]piperidine-1-carbonyl]phenyl]-1-[2-(difluoromethyl)-3-fluoro-4-(fluoromethoxy)phenyl]imidazole-4-carboxamide; 1:1 2,2,2-trifluoroacetic acid

A solution of 5-amino-N-[3-chloro-4-[4-[(3aS,6aR)-2,3,3a,4,6,6a-hexahydro-1H-pyrrolo[3,4-c]pyrrole-5-carbonyl]piperidine-1-carbonyl]phenyl]-1-[2-(difluoromethyl)-3-fluoro-4-(fluoromethoxy)phenyl]imidazole-4-carboxamide (50.0 mg, 0.07 mmol, 1.0 eq), polyformaldehyde (6.64 mg, 0.22 mmol, 3.0 eq), sodium acetate (18.15 mg, 0.22 mmol, 3.0 eq), and sodium cyanoborohydride (23.17 mg, 0.37 mmol, 5.0 eq) in methanol (3 mL) was stirred at 25° C. for 3 h. The resulting solution was concentrated under reduced pressure. The crude product was purified by Prep-HPLC (chiral Pak-AS, CO₂-MEOH (DEtOAc)) to afford 5-amino-N-[3-chloro-4-[4-[(3aS,6aR)-2-methyl-1,3,3a,4,6,6a-hexahydropyrrolo[3,4-c]pyrrole-5-carbonyl]piperidine-1-carbonyl]phenyl]-1-[2-(difluoromethyl)-3-fluoro-4-(fluoromethoxy)phenyl]imidazole-4-carboxamide; 1:1 2,2,2-trifluoroacetic acid (7.8 mg, 0.01 mmol, 13.0% yield). MS [M+H]⁺: 692.3.

### Compound 177

### N-[4-[4-[(3aS,6aR)-2-methyl-1,3,3a,4,6,6a-hexahydropyrrolo[3,4-c]pyrrole-5-carbonyl]piperidine-1-carbonyl]-3-chlorophenyl]-5-amino-1-[2,3-difluoro-4-(fluoromethoxy)phenyl]imidazole-4-carboxamide; 1:1 2,2,2-trifluoroacetic acid

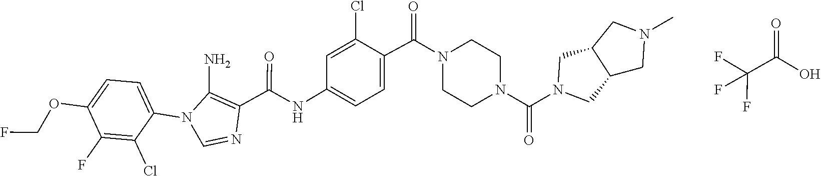 [SMILES: CN1C[C@@H]2CN(C(=O)N3CCN(C(=O)c4ccc(NC(=O)c5ncn(-c6ccc(OCF)c(F)c6Cl)c5N)cc4Cl)CC3)C[C@@H]2C1 ; O=C(O)C(F)(F)F]

### Step 1: tert-butyl (3aR,6aS)-2-[1-[4-[[5-amino-1-[2,3-difluoro-4-(fluoromethoxy)phenyl]imidazole-4-carbonyl]amino]-2-chlorobenzoyl]piperidine-4-carbonyl]-1,3,3a,4,6,6a-hexahydropyrrolo[3,4-c]pyrrole-5-carboxylate

A solution of tert-butyl (3aR,6aS)-2-[1-(4-amino-2-chlorobenzoyl)piperidine-4-carbonyl]-1,3,3a,4,6,6a-hexahydropyrrolo[3,4-c]pyrrole-5-carboxylate (100.0 mg, 0.21 mmol, 1.0 eq), 5-amino-1-[2,3-difluoro-4-(fluoromethoxy)phenyl]imidazole-4-carbonyl chloride (64.08 mg, 0.21 mmol, 1.0 eq) and pyridine (0.17 mL, 2.1 mmol, 10.0 eq) in methylene chloride (4 mL) was stirred at 20° C. for 3 h. The resulting solution was concentrated under reduced pressure. The residue was applied on a silica gel column and eluted with 1:20 MeOH/methylene chloride to afford tert-butyl (3aR,6aS)-2-[1-[4-[[5-amino-1-[2,3-difluoro-4-(fluoromethoxy)phenyl]imidazole-4-carbonyl]amino]-2-chlorobenzoyl]piperidine-4-carbonyl]-1,3,3a,4,6,6a-hexahydropyrrolo[3,4-c]pyrrole-5-carboxylate (150.0 mg, 0.2 mmol, 95.9% yield). MS [M+H]⁺: 746.2

### Step 2: N-[4-[4-[(3aS,6aR)-2,3,3a,4,6,6a-hexahydro-1H-pyrrolo[3,4-c]pyrrole-5-carbonyl]piperidine-1-carbonyl]-3-chlorophenyl]-5-amino-1-[2,3-difluoro-4-(fluoromethoxy)phenyl]imidazole-4-carboxamide

A solution of tert-butyl (3aR,6aS)-2-[1-[4-[[5-amino-1-[2,3-difluoro-4-(fluoromethoxy)phenyl]imidazole-4-carbonyl]amino]-2-chlorobenzoyl]piperidine-4-carbonyl]-1,3,3a,4,6,6a-hexahydropyrrolo[3,4-c]pyrrole-5-carboxylate (100.0 mg, 0.13 mmol, 1.0 eq) and trifluoroacetic acid (1.0 mL, 12.98 mmol, 96.85 eq) in methylene chloride (3 mL) was stirred at 20° C. for 1 h. The resulting solution was concentrated under reduced pressure to afford N-[4-[4-[(3aS,6aR)-2,3,3a,4,6,6a-hexahydro-1H-pyrrolo[3,4-c]pyrrole-5-carbonyl]piperidine-1-carbonyl]-3-chlorophenyl]-5-amino-1-[2,3-difluoro-4-(fluoromethoxy)phenyl]imidazole-4-carboxamide (80.0 mg, 92.4% yield). MS [M+H]⁺: 646.0

### Step 3: N-[4-[4-[(3aS,6aR)-2-methyl-1,3,3a,4,6,6a-hexahydropyrrolo[3,4-c]pyrrole-5-carbonyl]piperidine-1-carbonyl]-3-chlorophenyl]-5-amino-1-[2,3-difluoro-4-(fluoromethoxy)phenyl]imidazole-4-carboxamide; 1:1 2,2,2-trifluoroacetic acid

A solution of N-[4-[4-[(3aS,6aR)-2,3,3a,4,6,6a-hexahydro-1H-pyrrolo[3,4-c]pyrrole-5-carbonyl]piperidine-1-carbonyl]-3-chlorophenyl]-5-amino-1-[2,3-difluoro-4-(fluoromethoxy)phenyl]imidazole-4-carboxamide (50.0 mg, 0.08 mmol, 1.0 eq), polyformaldehyde (11.61 mg, 0.39 mmol, 5.0 eq), sodium acetate (0.06 mL, 0.77 mmol, 10.0 eq) and sodium cyanoborohydride (24.32 mg, 0.39 mmol, 5.0 eq) in methanol (3 mL) was stirred at 20° C. for 6 h. The resulting solution was concentrated under reduced pressure. The residue was purified by Prep-HPLC (Gemini-C18 150×19 mm, 5 μm, MeCN-H₂O (0.1% TFA), 35-45) to afford N-[4-[4-[(3aS,6aR)-2-methyl-1,3,3a,4,6,6a-hexahydropyrrolo[3,4-c]pyrrole-5-carbonyl]piperidine-1-carbonyl]-3-chlorophenyl]-5-amino-1-[2,3-difluoro-4-(fluoromethoxy)phenyl]imidazole-4-carboxamide; 1:1 2,2,2-trifluoroacetic acid (23.9 mg, 0.03 mmol, 46.6% yield). MS [M+H]⁺: 659.9

### Compound 178

### 5-amino-N-[3-chloro-4-[4-[(2R)-pyrrolidine-2-carbonyl]piperazine-1-carbonyl]phenyl]-1-[1-(2-fluoroallyl)-3-(trifluoromethyl)pyrazol-4-yl]imidazole-4-carboxamide;2,2,2-trifluoroacetic acid

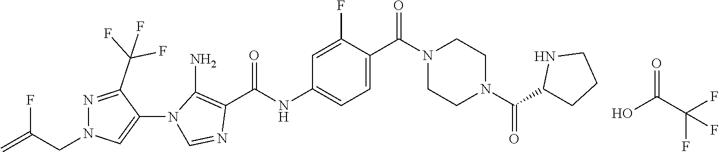

### Step 1: tert-butyl(2R)-2-[4-(2-chloro-4-nitrobenzoyl)piperazine-1-carbonyl]pyrrolidine-1-carboxylate

To a solution of (2-chloro-4-nitrophenyl)-piperazin-1-yl-methanone; hydrochloride (200.0 mg, 0.65 mmol, 1.0 eq) in methylene chloride (2 mL) were added BOC-D-Pro-OH (168.74 mg, 0.78 mmol, 1.2 eq), O-(7-azabenzotriazol-1-yl)-N,N,N′,N′-tetramethyluronium hexafluorophosphate (372.59 mg, 0.98 mmol, 1.5 eq) and N,N-diisopropylethylamine (168.86 mg, 1.31 mmol, 2.0 eq). The resulting mixture was stirred at 25° C. for 3 h. The solvent was removed under reduced pressure. The residue was diluted with EtOAc (20 mL), washed with H₂O (3×10 mL), dried over Na₂SO₄ and concentrated under reduced pressure. The residue was purified by flash column chromatography (1:4 petroleum ether:EtOAc) to give tert-butyl(2R)-2-[4-(2-chloro-4-nitrobenzoyl)piperazine-1-carbonyl]pyrrolidine-1-carboxylate (270.0 mg, 0.58 mmol, 88.5% yield). MS: [M+Na]⁺: 488.9

### Step 2: tert-butyl(2R)-2-[4-(4-amino-2-chlorobenzoyl)piperazine-1-carbonyl]pyrrolidine-1-carboxylate

To a solution of tert-butyl(2R)-2-[4-(2-chloro-4-nitrobenzoyl)piperazine-1-carbonyl]pyrrolidine-1-carboxylate (305.0 mg, 0.65 mmol, 1.0 eq) in ethanol (3 mL) were added iron powder (182.39 mg, 3.27 mmol, 5.0 eq), ammonium chloride (349.41 mg, 6.53 mmol, 10.0 eq) and water (1 mL) under N₂ atmosphere. The resulting mixture was stirred at 50° C. for 3 h. The solid was filtered off and EtOH was removed under reduced pressure. The residue was diluted with EtOAc (30 mL), washed with H₂O (3×15 mL), dried over Na₂SO₄ and concentrated under reduced pressure. The residue was purified by flash column chromatography (2:1 petroleum ether:EtOAc) to give tert-butyl(2R)-2-[4-(4-amino-2-chlorobenzoyl)piperazine-1-carbonyl]pyrrolidine-1-carboxylate (250.0 mg, 0.57 mmol, 81.5% yield). MS: [M−Boc+H]⁺: 337.0

### Step 3: 1-(2-fluoroallyl)-3-(trifluoromethyl)pyrazol-4-amine

To a solution of 1-(2-fluoroallyl)-4-nitro-3-(trifluoromethyl)pyrazole (11.42 g, 47.76 mmol, 1.0 eq) in ethanol (100 mL) were added iron powder (10.67 g, 191.03 mmol, 4.0 eq), ammonium chloride (20.44 g, 382.05 mmol, 8.0 eq) and water (30 mL) under N₂ atmosphere. The resulting mixture was stirred at 50° C. for 2 h. The solid was filtered off. The filtrate was diluted with EtOAc (80 mL), washed with water (3×25 mL), dried over Na₂SO₄ and evaporated under reduced pressure to give 1-(2-fluoroallyl)-3-(trifluoromethyl)pyrazol-4-amine (9678.0 mg, 46.28 mmol, 85.3% yield), the crude product was used directly without further purification. MS: [M+H]⁺: 210.1

### Step 4: ethyl 5-amino-1-[1-(2-fluoroallyl)-3-(trifluoromethyl)pyrazol-4-yl]imidazole-4-carboxylate

To a solution of ethyl 2-amino-2-cyanoacetate; 4-methylbenzene-1-sulfonic acid (27.8 g, 92.55 mmol, 2.0 eq) in triethyl orthoformate (170.0 mL, 1016.1 mmol, 21.96 eq) was added triethylamine (18.73 g, 185.1 mmol, 4.0 eq). The resulting mixture was stirred at 82° C. for 2 h. The solvent was removed under reduced pressure and the residue was diluted with MeCN (120 mL). Next, 1-(2-fluoroallyl)-3-(trifluoromethyl)pyrazol-4-amine (9.68 g, 46.28 mmol, 1.0 eq) was added and the resulting mixture was stirred at 25° C. for 15 h. The reaction was concentrated to dryness and the residue was taken up in EtOAc (100 mL). The organics were washed with 3×30 mL water followed by 30 mL brine. The organics were then separated and dried (Na₂SO₄) before concentrating to dryness. The crude was then purified by flash column chromatography eluting with 3% MeOH in methylene chloride to give ethyl 5-amino-1-[1-(2-fluoroallyl)-3-(trifluoromethyl)pyrazol-4-yl]imidazole-4-carboxylate (7800.0 mg, 22.46 mmol, 46.1% yield). MS: [M+H]⁺: 348.1

### Step 5: 5-amino-1-[1-(2-fluoroallyl)-3-(trifluoromethyl)pyrazol-4-yl]imidazole-4-carboxylic acid

To a solution of ethyl 5-amino-1-[1-(2-fluoroallyl)-3-(trifluoromethyl)pyrazol-4-yl]imidazole-4-carboxylate (3300.0 mg, 9.5 mmol, 1.0 eq) in isopropyl alcohol (50 mL) was added lithium hydroxide hydrate (saturated aqueous solution)(10.0 mL, 55.0 mmol, 5.79 eq) under N₂. The resulting mixture was stirred at 35° C. for 48 h. The solvent was removed under reduced pressure. The residue was diluted with H₂O (50 mL), extracted with EtOAc (3×40 mL) and the organic layers were discarded. The aqueous layer was acidified with HCl (4 N) pH to 5˜6 and filtered to provide 5-amino-1-[1-(2-fluoroallyl)-3-(trifluoromethyl)pyrazol-4-yl]imidazole-4-carboxylic acid (950.0 mg, 2.98 mmol, 18.79% yield), the crude product was used directly without further purification. MS [M+H]⁺: 320.0

### Step 6: 5-amino-1-[1-(2-fluoroallyl)-3-(trifluoromethyl)pyrazol-4-yl]imidazole-4-carbonyl chloride

A solution of 5-amino-1-[1-(2-fluoroallyl)-3-(trifluoromethyl)pyrazol-4-yl]imidazole-4-carboxylic acid (93.0 mg, 0.29 mmol, 1.0 eq) in thionyl chloride (2.0 mL, 27.54 mmol, 94.52 eq) was stirred under N₂ atmosphere at 25° C. for 1 h. Concentration under reduced pressure gave 5-amino-1-[1-(2-fluoroallyl)-3-(trifluoromethyl)pyrazol-4-yl]imidazole-4-carbonyl chloride (98.0 mg, 0.29 mmol, 63.8% yield), the crude product was used directly without further purification. MS [M−Cl+OCH₃+H]⁺: 334.0

### Step 7: tert-butyl(2R)-2-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]pyrrolidine-1-carboxylate

To a solution of tert-butyl(2R)-2-[4-(4-amino-2-chlorobenzoyl)piperazine-1-carbonyl]pyrrolidine-1-carboxylate (88.77 mg, 0.2 mmol, 0.7 eq) in methylene chloride (3 mL) were added pyridine (0.47 mL, 5.8 mmol, 20.0 eq) and 5-amino-1-[1-(2-fluoroallyl)-3-(trifluoromethyl)pyrazol-4-yl]imidazole-4-carbonyl chloride (98.0 mg, 0.29 mmol, 1.0 eq) in order under N₂ atmosphere. The resulting mixture was stirred at 25° C. for 1 h. The reaction mixture was then diluted with methylene chloride (20 mL), washed with H₂O (3×15 mL), dried over Na₂SO₄ and concentrated under reduced pressure. The residue was purified by flash column chromatography (94:6 methylene chloride:MeOH) to give tert-butyl(2R)-2-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]pyrrolidine-1-carboxylate (190.0 mg, 0.26 mmol, 49.7% yield). MS [M+H]⁺: 738.2

### Step 8: 5-amino-N-[3-chloro-4-[4-[(2R)-pyrrolidine-2-carbonyl]piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid

To a solution of tert-butyl(2R)-2-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]pyrrolidine-1-carboxylate (190.0 mg, 0.26 mmol, 1.0 eq) in dichloromethane (2 mL) was added trifluoroacetic acid (1.0 mL, 13.07 mmol, 50.77 eq) under N₂ atmosphere. The resulting mixture was stirred at 25° C. for 1 h. After concentration under reduced pressure, the residue was purified by prep-HPLC [Gemini-C18 150×21.2 mm, 5 μm, MeCN— H₂O (0.1% TFA), 25%˜35%] to give 5-amino-N-[3-chloro-4-[4-[(2R)-pyrrolidine-2-carbonyl]piperazine-1-carbonyl]phenyl]-1-[1-(2-fluoroallyl)-3-(trifluoromethyl)pyrazol-4-yl]imidazole-4-carboxamide; 2,2,2-trifluoroacetic acid (69.2 mg, 0.09 mmol, 35.0% yield). MS [M+H]⁺: 638.3

### Compound 179

### 5-amino-N-[3-chloro-4-[4-[(1S,5R,6r)-3-methyl-3-azabicyclo[3.1.0]hexane-6-carbonyl]piperazine-1-carbonyl]phenyl]-1-[1-cyclobutyl-3-(trifluoromethyl)pyrazol-4-yl]imidazole-4-carboxamide

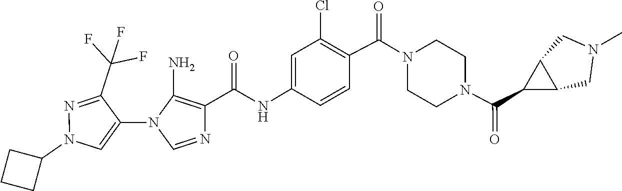 [SMILES: CN1C[C@@H]2[C@H](C1)[C@H]2C(=O)N1CCN(C(=O)c2ccc(NC(=O)c3ncn(-c4cn(C5CCC5)nc4C(F)(F)F)c3N)cc2Cl)CC1]

### Step 1: cyclobutyl 4-methylbenzenesulfonate

A solution of cyclobutanol (100.0 mg, 1.39 mmol, 1.0 eq), triethylamine (0.39 mL, 2.77 mmol, 2.0 eq), 4-dimethylaminopyridine (33.88 mg, 0.28 mmol, 0.2 eq) and p-toluenesulfonyl chloride (317.27 mg, 1.66 mmol, 1.2 eq) in methylene chloride (3 mL) was stirred at 25° C. for 15 h. The resulting solution was concentrated under reduced pressure. The residue applied on a silica gel column and eluted with 1:2 EtOAc:petroleum ether to give cyclobutyl 4-methylbenzenesulfonate (240.0 mg, 1.06 mmol, 76.5% yield). MS [M+H]⁺: 227.0

### Step 2: tert-butyl (1S,5R)-6-[4-[4-[[5-amino-1-[1-cyclobutyl-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of tert-butyl (1S,5R)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (100.0 mg, 0.14 mmol, 1.0 eq), cyclobutyl 4-methylbenzenesulfonate (49.04 mg, 0.22 mmol, 1.5 eq) and potassium carbonate (59.91 mg, 0.43 mmol, 3.0 eq) in DMF (2 mL) was stirred at 50° C. for 16 h. The resulting solution was diluted with 5 ml of water, then extracted with 3×5 mL of EtOAc. The organic layers were combined, dried and concentrated under reduced pressure. The residue was applied on a silica gel column and eluted with 10:1 methylene chloride:MeOH to give tert-butyl (1S,5R)-6-[4-[4-[[5-amino-1-[1-cyclobutyl-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (89.0 mg, 0.12 mmol, 82.6% yield). MS [M−t−Bu+H]⁺: 690.2

### Step 3: 5-amino-N-[4-[4-[(1R,5S)-3-azabicyclo[3.1.0]hexane-6-carbonyl]piperazine-1-carbonyl]-3-chlorophenyl]-1-[1-cyclobutyl-3-(trifluoromethyl)pyrazol-4-yl]imidazole-4-carboxamide

A solution of tert-butyl (1R,5S)-6-[4-[4-[[5-amino-1-[1-cyclobutyl-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (89.0 mg, 0.12 mmol, 1.0 eq) and trifluoroacetic acid (1 mL) in methylene chloride (4 mL) was stirred at 25° C. for 1 h. The resulting solution was concentrated under reduced pressure to give 5-amino-N-[4-[4-[(1R,5S)-3-azabicyclo[3.1.0]hexane-6-carbonyl]piperazine-1-carbonyl]-3-chlorophenyl]-1-[1-cyclobutyl-3-(trifluoromethyl)pyrazol-4-yl]imidazole-4-carboxamide (65.0 mg, 0.1 mmol, 84.4% yield) MS [M+H]⁺: 646.2

### Step 4: 5-amino-N-[3-chloro-4-[4-[(1S,5R)-3-methyl-3-azabicyclo[3.1.0]hexane-6-carbonyl]piperazine-1-carbonyl]phenyl]-1-[1-cyclobutyl-3-(trifluoromethyl)pyrazol-4-yl]imidazole-4-carboxamide

A solution of 5-amino-N-[4-[4-[(1R,5S)-3-azabicyclo[3.1.0]hexane-6-carbonyl]piperazine-1-carbonyl]-3-chlorophenyl]-1-[1-cyclobutyl-3-(trifluoromethyl)pyrazol-4-yl]imidazole-4-carboxamide (65.0 mg, 0.1 mmol, 1.0 eq), polyformaldehyde (9.06 mg, 0.3 mmol, 3.0 eq), sodium acetate (24.76 mg, 0.3 mmol, 3.0 eq), and sodium cyanoborohydride (31.61 mg, 0.5 mmol, 5.0 eq) in methanol (3 mL) was stirred at 25° C. for 15 h. The resulting solution was concentrated under reduced pressure. The crude product was purified by Prep-HPLC (Gemini C18 150×21.2 mm, 5 μm, MeCN— H₂O (0.1% TFA), 20-50) to afford 5-amino-N-[3-chloro-4-[4-[(1S,5R)-3-methyl-3-azabicyclo[3.1.0]hexane-6-carbonyl]piperazine-1-carbonyl]phenyl]-1-[1-cyclobutyl-3-(trifluoromethyl)pyrazol-4-yl]imidazole-4-carboxamide;2,2,2-trifluoroacetic acid (12.2 mg, 0.02 mmol, 15.5% yield). MS [M+H]⁺: 660.2

### Compound 180

### 5-amino-N-[3-chloro-4-[4-[(1S,5R,6r)-3-methyl-3-azabicyclo[3.1.0]hexane-6-carbonyl]piperazine-1-carbonyl]phenyl]-1-[1-[(1-fluorocyclopropyl)methyl]-3-(trifluoromethyl)pyrazol-4-yl]imidazole-4-carboxamide; 1:1 2,2,2-trifluoroacetic acid

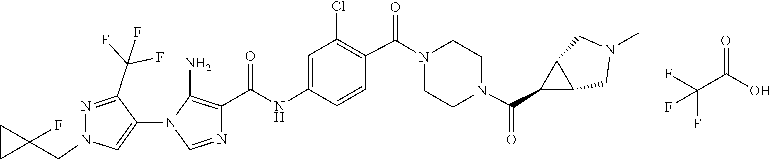 [SMILES: CN1C[C@@H]2[C@H](C1)[C@H]2C(=O)N1CCN(C(=O)c2ccc(NC(=O)c3ncn(-c4cn(CC5(F)CC5)nc4C(F)(F)F)c3N)cc2Cl)CC1 ; O=C(O)C(F)(F)F]

### Step 1: tert-butyl (1S,5R)-6-[4-[4-[[5-amino-1-[1-[(1-fluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl (1S,5R)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (550.0 mg, 0.79 mmol, 1.0 eq) in DMF (8 mL) was added potassium carbonate (366.11 mg, 2.65 mmol, 3.33 eq) and (1-fluorocyclopropyl)methyl 4-methylbenzenesulfonate (485.32 mg, 1.99 mmol, 2.5 eq). The reaction was stirred at 50° C. for 16 h. The reaction was concentrated to dryness and the residue was taken up in EtOAc (40 mL). The organics were washed with 2×20 mL water followed by 20 mL brine. The organics were then separated and dried (MgSO₄) before concentrating to dryness. The crude was then purified by flash column chromatography eluting with 10% MeOH in methylene chloride to afford tert-butyl (1S,5R)-6-[4-[4-[[5-amino-1-[1-[(1-fluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (513.0 mg, 0.67 mmol, 84.5% yield). MS [M−tBu+H]⁺: 708.1

### Step 2: 5-amino-N-[4-[4-[(1S,5R)-3-azabicyclo[3.1.0]hexane-6-carbonyl]piperazine-1-carbonyl]-3-chloro-phenyl]-1-[1-[(1-fluorocyclopropyl)methyl]-3-(trifluoromethyl)pyrazol-4-yl]imidazole-4-carboxamide; 1:1 2,2,2-trifluoroacetic acid

A solution of tert-butyl (1R,5S)-6-[4-[4-[[5-amino-1-[1-[(1-fluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (100.0 mg, 0.13 mmol, 1.0 eq) in methylene chloride (3 mL) was added trifluoroacetic acid (1.0 mL). The reaction was stirred at 25° C. for 3 h. The reaction was concentrated to afford 5-amino-N-[4-[4-[(1S,5R)-3-azabicyclo[3.1.0]hexane-6-carbonyl]piperazine-1-carbonyl]-3-chloro-phenyl]-1-[1-[(1-fluorocyclopropyl)methyl]-3-(trifluoromethyl)pyrazol-4-yl]imidazole-4-carboxamide; 1:1 2,2,2-trifluoroacetic acid (68.0 mg, 0.1 mmol, 73.2% yield). MS [M+H]⁺: 664.2

### Step 3: 5-amino-N-[3-chloro-4-[4-[(1S,5R)-3-methyl-3-azabicyclo[3.1.0]hexane-6-carbonyl]piperazine-1-carbonyl]phenyl]-1-[1-[(1-fluorocyclopropyl)methyl]-3-(trifluoromethyl)pyrazol-4-yl]imidazole-4-carboxamide; 1:1 2,2,2-trifluoroacetic acid

A solution of 5-amino-N-[4-[4-[(1S,5R)-3-azabicyclo[3.1.0]hexane-6-carbonyl]piperazine-1-carbonyl]-3-chlorophenyl]-1-[1-[(1-fluorocyclopropyl)methyl]-3-(trifluoromethyl)pyrazol-4-yl]imidazole-4-carboxamide (68.0 mg, 0.1 mmol, 1.0 eq), polyformaldehyde (9.23 mg, 0.31 mmol, 3.0 eq), sodium acetate (0.02 mL, 0.31 mmol, 3.0 eq), and sodium cyanoborohydride (32.17 mg, 0.51 mmol, 5.0 eq) in methanol (3 mL) was stirred at 25° C. for 15 h. The resulting solution was concentrated under reduced pressure. The crude product was purified by Prep-HPLC (Gemini C18 150×21.2 mm, 5 μm, MeCN-H₂O (0.1% TFA), 20-40) to afford 5-amino-N-[3-chloro-4-[4-[(1S,5R)-3-methyl-3-azabicyclo[3.1.0]hexane-6-carbonyl]piperazine-1-carbonyl]phenyl]-1-[1-[(1-fluorocyclopropyl)methyl]-3-(trifluoromethyl)pyrazol-4-yl]imidazole-4-carboxamide; 1:1 2,2,2-trifluoroacetic acid (27.1 mg, 0.03 mmol, 33.4% yield). MS [M+H]⁺: 678.0

### Compound 181

### 5-amino-N-[3-chloro-4-[4-[(1S,5R,6r)-3-methyl-3-azabicyclo[3.1.0]hexane-6-carbonyl]piperazine-1-carbonyl]phenyl]-1-[1-[(2,2-difluorocyclopropyl)methyl]-3-(trifluoromethyl)pyrazol-4-yl]imidazole-4-carboxamide; 1:1 2,2,2-trifluoroacetic acid

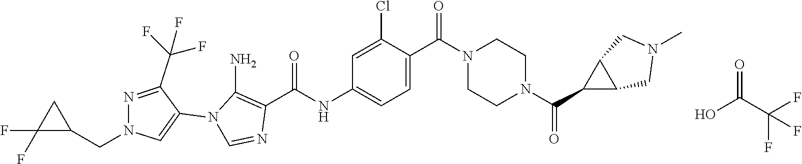 [SMILES: CN1C[C@@H]2[C@H](C1)[C@H]2C(=O)N1CCN(C(=O)c2ccc(NC(=O)c3ncn(-c4cn(CC5CC5(F)F)nc4C(F)(F)F)c3N)cc2Cl)CC1 ; O=C(O)C(F)(F)F]

### Step 1: tert-butyl (1R,5S)-6-[4-[4-[[5-amino-1-[1-[(2,2-difluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl (1R,5S)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (100.0 mg, 0.14 mmol, 1.0 eq) in DMF (3 mL) was added 1-bromomethyl-2,2-difluorocyclopropane (49.41 mg, 0.29 mmol, 2.0 eq) and potassium carbonate (59.91 mg, 0.43 mmol, 3.0 eq). The reaction was stirred at 25° C. for 16 h. The reaction was concentrated to dryness and the residue was taken up in EtOAc (20 mL). The organics were washed with 2×10 mL water followed by 10 mL brine. The organics were then separated and dried (MgSO₄) before concentrating to dryness to afford product tert-butyl (1R,5S)-6-[4-[4-[[5-amino-1-[1-[(2,2-difluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (91.0 mg, 0.12 mmol, 80.5% yield). MS [M-Bu+H]⁺: 725.9

### Step 2: 5-amino-N-[4-[4-[(1S,5R)-3-azabicyclo[3.1.0]hexane-6-carbonyl]piperazine-1-carbonyl]-3-chlorophenyl]-1-[1-[(2,2-difluorocyclopropyl)methyl]-3-(trifluoromethyl)pyrazol-4-yl]imidazole-4-carboxamide; 1:1 2,2,2-trifluoroacetic acid

To a solution of tert-butyl (1R,5S)-6-[4-[4-[[5-amino-1-[1-[(2,2-difluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (91.0 mg, 0.12 mmol, 1.0 eq) in methylene chloride (3 mL) was added trifluoroacetic acid (1.0 mL). The reaction was stirred at 25° C. for 3 h. The reaction was concentrated to afford 5-amino-N-[4-[4-[(1S,5R)-3-azabicyclo[3.1.0]hexane-6-carbonyl]piperazine-1-carbonyl]-3-chlorophenyl]-1-[1-[(2,2-difluorocyclopropyl)methyl]-3-(trifluoromethyl)pyrazol-4-yl]imidazole-4-carboxamide; 1:1 2,2,2-trifluoroacetic acid (63.0 mg, 0.09 mmol, 79.4% yield). MS [M+H]⁺: 681.9

### Step 3: 5-amino-N-[3-chloro-4-[4-[(1S,5R)-3-methyl-3-azabicyclo[3.1.0]hexane-6-carbonyl]piperazine-1-carbonyl]phenyl]-1-[1-[(2,2-difluorocyclopropyl)methyl]-3-(trifluoromethyl)pyrazol-4-yl]imidazole-4-carboxamide; 1:1 2,2,2-trifluoroacetic acid

A solution of 5-amino-N-[4-[4-[(1S,5R)-3-azabicyclo[3.1.0]hexane-6-carbonyl]piperazine-1-carbonyl]-3-chlorophenyl]-1-[1-[(2,2-difluorocyclopropyl)methyl]-3-(trifluoromethyl)pyrazol-4-yl]imidazole-4-carboxamide (63.0 mg, 0.09 mmol, 1.0 eq), polyformaldehyde (8.32 mg, 0.28 mmol, 3.0 eq), sodium acetate (0.02 mL, 0.28 mmol, 3.0 eq), and sodium cyanoborohydride (29.02 mg, 0.46 mmol, 5.0 eq) in methanol (3 mL) was stirred at 25° C. for 12 h. The resulting solution was concentrated under reduced pressure. The crude product was purified by Prep-HPLC (Gemini C18 150×21.2 mm, 5 μm, MeCN-H₂O (0.1% TFA), 15-40) to afford 5-amino-N-[3-chloro-4-[4-[(1S,5R)-3-methyl-3-azabicyclo[3.1.0]hexane-6-carbonyl]piperazine-1-carbonyl]phenyl]-1-[1-[(2,2-difluorocyclopropyl)methyl]-3-(trifluoromethyl)pyrazol-4-yl]imidazole-4-carboxamide; 1:1 2,2,2-trifluoroacetic acid (43.7 mg, 0.05 mmol, 67.6% yield). MS [M+H]⁺: 696.2

### Compound 182

### 5-amino-1-[1-(2-chloroallyl)-3-(trifluoromethyl)pyrazol-4-yl]-N-[3-chloro-4-[4-[(1S,5R,6r)-3-(2-hydroxyethyl)-3-azabicyclo[3.1.0]hexane-6-carbonyl]piperazine-1-carbonyl]phenyl]imidazole-4-carboxamide; 1:1 2,2,2-trifluoroacetic acid

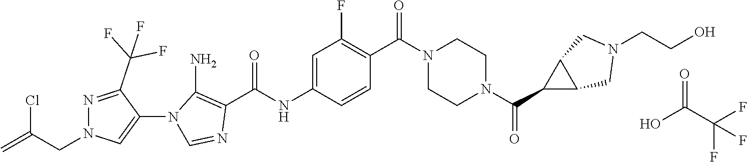

### Step 1: 2-chloroallyl 4-methylbenzenesulfonate

To a solution of 2-chloro-2-propen-1-ol (1000.0 mg, 10.81 mmol, 1.0 eq) in methylene chloride (5 mL) was added p-toluenesulfonyl chloride (3090.95 mg, 16.21 mmol, 1.5 eq), triethylamine (4.52 mL, 32.43 mmol, 3.0 eq) and 4-dimethylaminopyridine (132.04 mg, 1.08 mmol, 0.1 eq). The reaction was stirred at 25° C. for 16 h. The reaction was concentrated to dryness to give a crude product. The crude was purified by flash column chromatography eluting with 10% EtOAc/petroleum ether to afford 2-chloroallyl 4-methylbenzenesulfonate (850.0 mg, 3.45 mmol, 31.9% yield). MS [M+H]⁺: 247.0

### Step 2: tert-butyl (1S,5R)-6-[4-[4-[[5-amino-1-[1-(2-chloroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl (1S,5R)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (100.0 mg, 0.14 mmol, 1.0 eq) in DMF (2 mL) was added potassium carbonate (66.56 mg, 0.48 mmol, 3.33 eq) and 2-chloroallyl 4-methylbenzenesulfonate (89.12 mg, 0.36 mmol, 2.5 eq). The reaction was stirred at 50° C. for 2 h. The reaction was concentrated to dryness and the residue was taken up in EtOAc (20 mL). The organics were washed with 2×20 mL water followed by 20 mL brine. The organics were then separated and dried (MgSO₄) before concentrating to dryness. The crude was then purified by flash column chromatography eluting with with 10% MeOH in methylene chloride to afford tert-butyl (1S,5R)-6-[4-[4-[[5-amino-1-[1-(2-chloroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (65.0 mg, 0.08 mmol, 58.7% yield). MS [M+H]⁺: 767.3.

### Step 3: 5-[2-chloro-3-fluoro-4-[1-(2-methoxyethyl)-5-methyl-pyrazol-4-yl]phenyl]-4-[(2,2,2-trifluoroacetyl)amino]isothiazole-3-carboxylic acid

A solution of tert-butyl 1S,5R)-6-[4-[4-[[5-amino-1-[1-(2-chloroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (55.0 mg, 0.07 mmol, 1.0 eq) and trifluoroacetic acid (0.25 mL, 0.07 mmol, 1.0 eq) in methylene chloride (1 mL) was stirred at 25° C. for 1 h. After quenching the reaction, the organic layer was evaporated to dryness to afford 5-amino-1-[1-(2-chloroallyl)-3-(trifluoromethyl) pyrazol-4-yl]-N-[3-chloro-4-[4-[(1S,5R)-3-azabicyclo[3.1.0]hexane-6-carbonyl]piperazine-1-carbonyl]phenyl]imidazole-4-carboxamide (50.0 mg, 0.08 mmol, 95.3% yield). MS [M+H]⁺: 666.3.

### Step 4: 5-amino-1-[1-(2-chloroallyl)-3-(trifluoromethyl)pyrazol-4-yl]-N-[3-chloro-4-[4-[(1S,5R)-3-(2-hydroxyethyl)-3-azabicyclo[3.1.0]hexane-6-carbonyl]piperazine-1-carbonyl]phenyl]imidazole-4-carboxamide; 1:1 2,2,2-trifluoroacetic acid

A solution of 5-amino-1-[1-(2-chloroallyl)-3-(trifluoromethyl)pyrazol-4-yl]-N-[3-chloro-4-[4-[(1S,5R)-3-azabicyclo[3.1.0]hexane-6-carbonyl]piperazine-1-carbonyl]phenyl]imidazole-4-carboxamide (40.0 mg, 0.06 mmol, 1.0 eq), sodium acetate (14.77 mg, 0.18 mmol, 3.0 eq), glycolaldehyde (7.21 mg, 0.12 mmol, 2.0 eq) and sodium cyanoborohydride (18.86 mg, 0.3 mmol, 5.0 eq) in methanol (2 mL) was stirred at 25° C. for 1 h. The resulting solution was concentrated under reduced pressure. The residue was purified by Prep-HPLC (Gemini-C18 150×21.2 mm, 5 μm, MeCN-H₂O (0.1% TFA), 35-40) to give crude. The crude was purified by chiral-HPLC (chiralpak-OJ, CO₂-MeOH(EtOAc)) to give 5-amino-1-[1-(2-chloroallyl)-3-(trifluoromethyl)pyrazol-4-yl]-N-[3-chloro-4-[4-[(1S,5R)-3-(2-hydroxyethyl)-3-azabicyclo[3.1.0]hexane-6-carbonyl]piperazine-1-carbonyl]phenyl]imidazole-4-carboxamide; 1:1 2,2,2-trifluoroacetic acid (12.4 mg, 0.02 mmol, 29.1% yield). MS [M+H]⁺: 709.9

### Compound 183

### 5-amino-N-[4-[4-[(1R,5S,6r)-3-azabicyclo[3.1.0]hexane-6-carbonyl]piperazine-1-carbonyl]-3-chlorophenyl]-1-[4-(dimethylamino)-2,3-difluorophenyl]imidazole-4-carboxamide; 1:1 2,2,2-trifluoroacetic acid

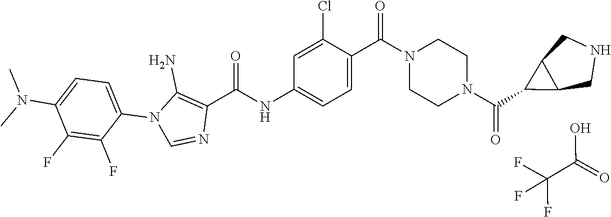

### Step 1: tert-butyl (1R,5S)-6-[4-[4-[[5-amino-1-[4-(dimethylamino)-2,3-difluorophenyl]imidazole-4-carbonyl]amino]-2-chlorobenzoyl]piperazine-1-carbonyl]-3-azabicyclo[3.1.0]hexane-3-carboxylate

To a solution of tert-butyl (1R,5S)-6-[4-(4-amino-2-chlorobenzoyl)piperazine-1-carbonyl]-3-azabicyclo[3.1.0]hexane-3-carboxylate (52.26 mg, 0.12 mmol, 0.7 eq) and pyridine (0.27 mL, 3.33 mmol, 20.0 eq) in methylene chloride (1 mL) was added a suspension of 5-amino-1-[4-(dimethylamino)-2,3-difluorophenyl]imidazole-4-carbonyl chloride (50.0 mg, 0.17 mmol, 1.0 eq) in methylene chloride (1 mL) at 25° C. The mixture was stirred at 25° C. for 1 h under N₂. The resulting solution was evaporated under reduced pressure. The residue was purified by column chromatography (silica gel, 0 to 10% MeOH in methylene chloride) to give tert-butyl (1R,5S)-6-[4-[4-[[5-amino-1-[4-(dimethylamino)-2,3-difluorophenyl]imidazole-4-carbonyl]amino]-2-chlorobenzoyl]piperazine-1-carbonyl]-3-azabicyclo[3.1.0]hexane-3-carboxylate (59.0 mg, 0.08 mmol, 32.3% yield). MS [M+H]⁺: 713.3.

### Step 2: 5-amino-N-[4-[4-[(1R,5S)-3-azabicyclo[3.1.0]hexane-6-carbonyl]piperazine-1-carbonyl]-3-chlorophenyl]-1-[4-(dimethylamino)-2,3-difluoro-phenyl]imidazole-4-carboxamide; 1:1 2,2,2-trifluoroacetic acid

A solution of tert-butyl (1R,5S)-6-[4-[4-[[5-amino-1-[4-(dimethylamino)-2,3-difluorophenyl]imidazole-4-carbonyl]amino]-2-chlorobenzoyl]piperazine-1-carbonyl]-3-azabicyclo[3.1.0]hexane-3-carboxylate (31.17 mg, 0.04 mmol, 1.0 eq) and trifluoroacetic acid (4.98 mg, 0.04 mmol, 1.0 eq) in methylene chloride (1 mL) was stirred at 25° C. for 1 h. The resulting solution was concentrated under reduced pressure. The crude product was purified by Prep-HPLC (Gemini-C18 150×21.2 mm, 5 μm, MeCN-H₂O (0.1% TFA), 15-45) to give 5-amino-N-[4-[4-[(1R,5S)-3-azabicyclo[3.1.0]hexane-6-carbonyl]piperazine-1-carbonyl]-3-chlorophenyl]-1-[4-(dimethyl-amino)-2,3-difluorophenyl]imidazole-4-carboxamide; 1:1 2,2,2-trifluoroacetic acid (14.5 mg, 0.02 mmol, 52.7% yield). MS [M+H]⁺: 613.8.

### Compound 184

### 5-amino-N-[3-chloro-4-[4-(piperidine-4-carbonyl)piperazine-1-carbonyl]phenyl]-1-[4-(dimethylamino)-2,3-difluorophenyl]imidazole-4-carboxamide; 1:1 2,2,2-trifluoroacetic acid

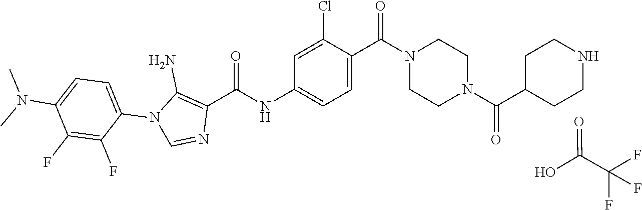

### Step 1: ethyl 5-amino-1-(4-bromo-2,3-difluorophenyl) imidazole-4-carboxylate

To a solution of ethyl 2-amino-2-cyanoacetate; 4-methylbenzene-1-sulfonic acid (14.4 g, 48.1 mmol, 2.0 eq) in triethyl orthoformate (154.6 g, 1043 mmol, 43.4 eq) was added triethylamine (9.7 g, 96.15 mmol, 4.0 eq). The resulting mixture was stirred at 82° C. for 2 h. The solvent was removed under reduced pressure and the residue was diluted with MeCN (20 mL). Next, 4-bromo-2,3-difluoroaniline (5.0 g, 24.04 mmol, 1.0 eq) was added and the resulting mixture was stirred at 45° C. for 15 h. The reaction was concentrated to dryness and the residue was taken up in EtOAc (30 mL). After washing with 2×30 mL water and 30 mL brine, the organics were separated and dried (MgSO₄) before concentrating to dryness. The crude material was then purified by flash column chromatography eluting with 3% MeOH in methylene chloride to yield ethyl 5-amino-1-(4-bromo-2,3-difluorophenyl) imidazole-4-carboxylate (4.5 mg, 0.01 mmol, 0.05% yield). MS [M+H]⁺: 345.9.

### Step 2: ethyl 5-amino-1-[4-(dimethylamino)-2,3-difluorophenyl]imidazole-4-carboxylate

To a solution of ethyl 5-amino-1-(4-bromo-2,3-difluorophenyl) imidazole-4-carboxylate (500.0 mg, 1.44 mmol, 1.0 eq) in 1,4-dioxane (30 mL) were added dimethylamine (2.17 mL, 4.33 mmol, 3.0 eq), palladium, [1,3-bis[2,6-bis(1-propylbutyl)phenyl]-4,5-dichloro-1,3-dihydro-2H-imidazol-2-ylidene]dichloro(3-chloropyridine-κN)-, (SP-4-1)-(268.04 mg, 0.29 mmol, 0.2 eq) and cesium carbonate (2823.97 mg, 8.67 mmol, 6.0 eq) under N₂ atmosphere. The resulting mixture was stirred at 100° C. for 15 h. The solvent was removed under reduced pressure, diluted with EtOAc (50 mL) and washed with H₂O (3×20 mL), dried over Na₂SO₄ and concentrated under reduced pressure and the residue was purified by flash column chromatography (1:9 petroleum ether:EtOAc) to give ethyl 5-amino-1-[4-(dimethylamino)-2,3-difluorophenyl]imidazole-4-carboxylate (310.0 mg, 1.0 mmol, 65.7% yield). MS [M+H]⁺: 311.1.

### Step 3: 5-amino-1-[4-(dimethylamino)-2,3-difluorophenyl]imidazole-4-carboxylic acid

To a solution of ethyl 5-amino-1-[4-(dimethylamino)-2,3-difluorophenyl]imidazole-4-carboxylate (310.0 mg, 1.0 mmol, 1.0 eq) in isopropyl alcohol (5 mL) were added NaOH (199.79 mg, 5.0 mmol, 5.0 eq) and water (1.5 mL). The resulting mixture was stirred at 50° C. for 8 h. The solvent was removed under vacuo, then diluted with H₂O (15 mL), extracted with EtOAc (10 mL×3) and the organic layers were discarded, the aqueous layer was acidified with HCl (4 N), then extracted with EtOAc, dried over Na₂SO₄ and concentrated to afford 5-amino-1-[4-(dimethylamino)-2,3-difluorophenyl]imidazole-4-carboxylic acid (90.0 mg, 0.32 mmol, 31.9% yield). The crude was used directly without further purification. MS [M+H]⁺: 283.1.

### Step 4: 5-amino-1-[4-(dimethylamino)-2,3-difluorophenyl]imidazole-4-carbonyl chloride

A solution of 5-amino-1-[4-(dimethylamino)-2,3-difluorophenyl]imidazole-4-carboxylic acid (32.0 mg, 0.11 mmol, 1.0 eq) in thionyl chloride (2.42 mL, 33.4 mmol, 295 eq) was stirred at 25° C. for 1 h. The resulting solution was concentrated under reduced pressure to give 5-amino-1-[4-(dimethylamino)-2,3-difluorophenyl]imidazole-4-carbonyl chloride (32.0 mg, 0.11 mmol, 78.8% yield). MS [(M−Cl+OCH₃)+H]⁺: 297.2.

### Step 5: tert-butyl 4-[4-[4-[[5-amino-1-[4-(dimethylamino)-2,3-difluorophenyl]imidazole-4-carbonyl]amino]-2-chlorobenzoyl]piperazine-1-carbonyl]piperidine-1-carboxylate

To a solution of tert-butyl 4-[4-[4-[[5-amino-1-[4-(dimethylamino)-2,3-difluorophenyl]imidazole-4-carbonyl]amino]-2-chlorobenzoyl]piperazine-1-carbonyl]piperidine-1-carboxylate (33.59 mg, 0.07 mmol, 0.7 eq) and pyridine (0.17 mL, 2.13 mmol, 20.0 eq) in methylene chloride (1 mL) was added a suspension of 5-amino-1-[4-(dimethylamino)-2,3-difluorophenyl]imidazole-4-carbonyl chloride (32.0 mg, 0.11 mmol, 1.0 eq) in methylene chloride (1 mL) at 25° C. The mixture was stirred at 25° C. for 1 h under N₂. The resulting solution was evaporated under reduced pressure. The residue was purified by column chromatography (silica gel, 0 to 10% MeOH in methylene chloride) to give tert-butyl 4-[4-[4-[[5-amino-1-[4-(dimethylamino)-2,3-difluorophenyl]imidazole-4-carbonyl]amino]-2-chlorobenzoyl]piperazine-1-carbonyl]piperidine-1-carboxylate (46.0 mg, 0.06 mmol, 46.5% yield). MS [M+H]⁺: 715.2.

### Step 6: 5-amino-N-[3-chloro-4-[4-(piperidine-4-carbonyl)piperazine-1-carbonyl]phenyl]-1-[4-(dimethylamino)-2,3-difluorophenyl]imidazole-4-carboxamide; 1:1 2,2,2-trifluoroacetic acid

A solution of tert-butyl 4-[4-[4-[[5-amino-1-[4-(dimethylamino)-2,3-difluorophenyl]imidazole-4-carbonyl]amino]-2-chlorobenzoyl]piperazine-1-carbonyl]piperidine-1-carboxylate (36.0 mg, 0.05 mmol, 1.0 eq) and trifluoroacetic acid (1.0 mL, 1.0 eq) in methylene chloride (1 mL) was stirred at 25° C. for 1 h. The resulting solution was concentrated under reduced pressure. The crude product was purified by Prep-HPLC (Gemini-C18 150×21.2 mm, 5 μm, MeCN-H₂O (0.1% TFA), 15-45) to give 5-amino-N-[3-chloro-4-[4-(piperidine-4-carbonyl)piperazine-1-carbonyl]phenyl]-1-[4-(dimethylamino)-2,3-difluorophenyl]imidazole-4-carboxamide; 1:1 2,2,2-trifluoroacetic acid (32.5 mg, 0.04 mmol, 87.3% yield). MS [M+H]⁺: 615.2.

### Compound 185

### 5-amino-N-[3-chloro-4-[4-(piperazine-1-carbonyl)piperazine-1-carbonyl]phenyl]-1-[4-(cyclopropylamino)-2,3-difluorophenyl]imidazole-4-carboxamide; 1:1 2,2,2-trifluoroacetic acid

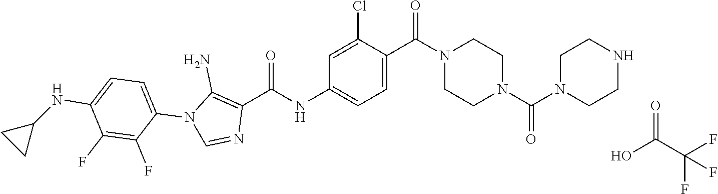

### Step 1: ethyl 5-amino-1-(4-bromo-2,3-difluorophenyl) imidazole-4-carboxylate

To a solution of ethyl 2-amino-2-cyanoacetate; 4-methylbenzene-1-sulfonic acid (11.55 g, 38.46 mmol, 2.0 eq) in triethyl orthoformate (28.5 g, 192.31 mmol, 10.0 eq) was added triethylamine (10.72 mL, 76.92 mmol, 4.0 eq). The resulting mixture was stirred at 80° C. for 2 h. The solvent was removed under reduced pressure and the residue was diluted with MeCN (40 mL), and then 4-bromo-2,3-difluoroaniline (4.0 g, 19.23 mmol, 1.0 eq) was added. The resulting mixture was stirred at 40° C. for 15 h. After concentration under reduced pressure, the residue was purified by flash column chromatography on silica gel (ethyl acetate:petroleum ether 60:40) to give ethyl 5-amino-1-(4-bromo-2,3-difluorophenyl) imidazole-4-carboxylate (4.2 g, 12.13 mmol, 54.9% yield). MS [M+H]⁺: 346.0

### Step 2: ethyl 5-amino-1-[4-(cyclopropylamino)-2,3-difluorophenyl]imidazole-4-carboxylate

To a solution of ethyl 5-amino-1-(4-bromo-2,3-difluorophenyl) imidazole-4-carboxylate (600.0 mg, 1.73 mmol, 1.0 eq) in 1,4-dioxane (10 mL) were added cyclopropylamine (296.94 mg, 5.2 mmol, 3.0 eq), cesium carbonate (1694.38 mg, 5.2 mmol, 3.0 eq) and palladium, [1,3-bis[2,6-bis(1-propylbutyl)phenyl]-4,5-dichloro-1,3-dihydro-2H-imidazol-2-ylidene]dichloro(3-chloropyridine-κN)-, (SP-4-1)-(337.25 mg, 0.35 mmol, 0.2 eq) under N₂ atmosphere. The resulting mixture was stirred at 80° C. for 8 h. The resulting solution was concentrated under reduced pressure. The residue was applied on a silica gel column and eluted with 1:1 ethyl acetate:hexane to give ethyl 5-amino-1-[4-(cyclopropylamino)-2,3-difluoro-phenyl]imidazole-4-carboxylate (370.0 mg, 1.15 mmol, 55.6% yield). MS [M+H]⁺: 323.1

### Step 3: 5-amino-1-[4-(cyclopropylamino)-2,3-difluorophenyl]imidazole-4-carboxylic acid

Ethyl 5-amino-1-[4-(cyclopropylamino)-2,3-difluorophenyl]imidazole-4-carboxylate (370.0 mg, 1.15 mmol, 1.0 eq) was dissolved in isopropyl alcohol (3 mL). A solution of sodium hydroxide (229.57 mg, 5.74 mmol, 5.0 eq) in water (1.5 mL) was added dropwise at 25° C. and stirred at 50° C. for 6 h. The solvent was then evaporated, and the residue was diluted with water (10 mL). The solution was extracted with ethyl acetate (2×10 mL). The aqueous layer was acidified to pH 5 with HCl (2 M), and the product was collected by filtration to give 5-amino-1-[4-(cyclopropylamino)-2,3-difluorophenyl]imidazole-4-carboxylic acid (240.0 mg, 0.82 mmol, 63.2% yield). MS [M−H]⁺: 295.0

### Step 4: 5-amino-1-[4-(cyclopropylamino)-2,3-difluorophenyl]imidazole-4-carbonyl chloride

To 5-amino-1-[4-(cyclopropylamino)-2,3-difluorophenyl]imidazole-4-carboxylic acid (80.0 mg, 0.27 mmol, 1.0 eq) under N₂ was added thionyl chloride (4.57 mL, 63.02 mmol, 231.79 eq) at 25° C. The solution was stirred at 25° C. for 1 h under N₂. The resulting solution was evaporated to dryness to give 5-amino-1-[4-(cyclopropylamino)-2,3-difluorophenyl]imidazole-4-carbonyl chloride (80.0 mg, 0.26 mmol, 79.0% yield), which was used without further purification. MS [M−Cl+OCH₃+H]⁺: 309.1

### Step 5: tert-butyl 4-[4-[4-[[5-amino-1-[4-(cyclopropylamino)-2,3-difluorophenyl]imidazole-4-carbonyl]amino]-2-chlorobenzoyl]piperazine-1-carbonyl]piperazine-1-carboxylate

A solution of tert-butyl 4-[4-(4-amino-2-chlorobenzoyl)piperazine-1-carbonyl]piperazine-1-carboxylate (92.5 mg, 0.2 mmol, 0.8 eq) and pyridine (0.41 mL, 5.12 mmol, 20.0 eq) in methylene chloride (2 mL) was stirred at 25° C. Next a solution of 5-amino-1-[4-(cyclopropylamino)-2,3-difluorophenyl]imidazole-4-carbonyl chloride (80.0 mg, 0.26 mmol, 1.0 eq) in methylene chloride (2 mL) was added, and the mixture was stirred at 25° C. for 1 h under N₂. The resulting solution was concentrated under reduced pressure. The residue was applied on a silica gel column and eluted with 10:1 methylene chloride:MeOH to give tert-butyl 4-[4-[4-[[5-amino-1-[4-(cyclopropylamino)-2,3-difluorophenyl]imidazole-4-carbonyl]amino]-2-chlorobenzoyl]piperazine-1-carbonyl]piperazine-1-carboxylate (95.0 mg, 0.13 mmol, 41.8% yield). MS [M+H]⁺: 728.2.

### Step 6: 5-amino-N-[3-chloro-4-[4-(piperazine-1-carbonyl)piperazine-1-carbonyl]phenyl]-1-[4-(cyclopropylamino)-2,3-difluorophenyl]imidazole-4-carboxamide; 1:1 2,2,2-trifluoroacetic acid

A solution of tert-butyl 4-[4-[4-[[5-amino-1-[4-(cyclopropylamino)-2,3-difluorophenyl]imidazole-4-carbonyl]amino]-2-chlorobenzoyl]piperazine-1-carbonyl]piperazine-1-carboxylate (95.0 mg, 0.13 mmol, 1.0 eq), trifluoroacetic acid (1.5 mL, 19.46 mmol, 149.2 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 30-60) to afford 5-amino-N-[3-chloro-4-[4-(piperazine-1-carbonyl)piperazine-1-carbonyl]phenyl]-1-[4-(cyclopropylamino)-2,3-difluorophenyl]imidazole-4-carboxamide; 1:1 2,2,2-trifluoroacetic acid (49.5 mg, 0.07 mmol, 70.7% yield). MS [M+H]⁺: 628.3

### Compound 186

### 5-amino-N-[3-chloro-4-[4-(piperazine-1-carbonyl)piperazine-1-carbonyl]phenyl]-1-[2,3-difluoro-4-(methylamino)phenyl]imidazole-4-carboxamide; 1:1 2,2,2-trifluoroacetic acid

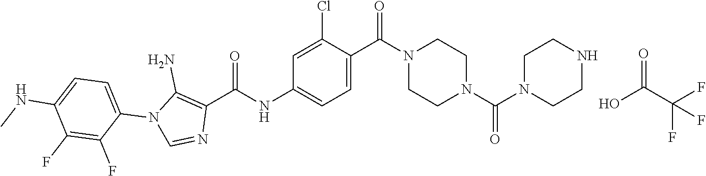

### Step 1: 5-amino-1-(4-bromo-2,3-difluorophenyl) imidazole-4-carboxylic acid

Ethyl 5-amino-1-(4-bromo-2,3-difluorophenyl) imidazole-4-carboxylate (1.1 g, 3.18 mmol, 1.0 eq) was dissolved in isopropyl alcohol (16 mL). A solution of sodium hydroxide (635.55 mg, 15.89 mmol, 5.0 eq) in water (8 mL) was added dropwise at 25° C. and the reaction mixture was stirred at 50° C. for 6 h. Solvent was then evaporated. The residue was diluted with water (20 mL), and the solution was extracted with ethyl acetate (2×50 mL). The aqueous layer was acidified to pH 5 with HCl (2 M), and the product was collected by filtration to give 5-amino-1-(4-bromo-2,3-difluorophenyl) imidazole-4-carboxylic acid (720.0 mg, 2.26 mmol, 71.2% yield). MS [M+H]⁺: 317.9

### Step 2: 5-amino-1-(4-bromo-2,3-difluorophenyl) imidazole-4-carbonyl chloride

5-amino-1-(4-bromo-2,3-difluorophenyl) imidazole-4-carboxylic acid (1083.0 mg, 3.4 mmol, 1.0 eq) was dissolved in thionyl chloride (8.0 mL, 110.28 mmol, 32.39 eq) at 25° C. under N₂. The reaction mixture was stirred at 25° C. for 1 h under N₂. The resulting solution was evaporated to dryness to give the title compound 5-amino-1-(4-bromo-2,3-difluorophenyl) imidazole-4-carbonyl chloride (1143.0 mg, 3.4 mmol, 79.8% yield), which was used without further purification. MS [M−Cl+OCH₃+H]⁺: 331.9

### Step 3: tert-butyl 4-[4-[4-[[5-amino-1-(4-bromo-2,3-difluorophenyl) imidazole-4-carbonyl]amino]-2-chlorobenzoyl]piperazine-1-carbonyl]piperazine-1-carboxylate

A solution of tert-butyl 4-[4-(4-amino-2-chlorobenzoyl)piperazine-1-carbonyl]piperazine-1-carboxylate (859.63 mg, 1.9 mmol, 0.7 eq) and pyridine (2.2 mL, 27.17 mmol, 10.0 eq) in methylene chloride (20 mL) was stirred at 25° C. This was followed by the addition of 5-amino-1-(4-bromo-2,3-difluoro-phenyl) imidazole-4-carbonyl chloride (1143.0 mg, 2.72 mmol, 1.0 eq) in methylene chloride (10 mL). The reaction mixture was stirred at 25° C. for 1 h under N₂. The resulting solution was concentrated under reduced pressure. The residue was applied on a silica gel column and eluted with methylene chloride/MeOH (92:8) to give tert-butyl 4-[4-[4-[[5-amino-1-(4-bromo-2,3-difluorophenyl) imidazole-4-carbonyl]amino]-2-chlorobenzoyl]piperazine-1-carbonyl]piperazine-1-carboxylate (1360.0 mg, 1.81 mmol, 66.6% yield). MS [M+H]⁺: 750.8

### Step 4: tert-butyl 4-[4-[4-[[5-amino-1-[2,3-difluoro-4-(methylamino)phenyl]imidazole-4-carbonyl]amino]-2-chlorobenzoyl]piperazine-1-carbonyl]piperazine-1-carboxylate

To a solution of tert-butyl 4-[4-[4-[[5-amino-1-(4-bromo-2,3-difluorophenyl) imidazole-4-carbonyl]amino]-2-chlorobenzoyl]piperazine-1-carbonyl]piperazine-1-carboxylate (100.0 mg, 0.13 mmol, 1.0 eq) in 1,4-dioxane (2 mL) were added methylamine hydrochloride (17.96 mg, 0.27 mmol, 2.0 eq), cesium carbonate (216.63 mg, 0.66 mmol, 5.0 eq) and palladium, [1,3-bis[2,6-bis(1-propylbutyl)phenyl]-4,5-dichloro-1,3-dihydro-2H-imidazol-2-ylidene]dichloro(3-chloropyridine-κN)-, (SP-4-1)-(12.94 mg, 0.01 mmol, 0.1 eq) under N₂. The resulting mixture was stirred at 100° C. for 16 h and then diluted with EtOAc (30 mL). The solution was then washed with H₂O (3×20 mL), dried over Na₂SO₄ and concentrated under reduced pressure. The residue was purified by flash column chromatography on silica gel (95:5 methylene chloride:MeOH) to give tert-butyl 4-[4-[4-[[5-amino-1-[2,3-difluoro-4-(methylamino)phenyl]imidazole-4-carbonyl]amino]-2-chlorobenzoyl]piperazine-1-carbonyl]piperazine-1-carboxylate (77.0 mg, 0.11 mmol, 75.0% yield). MS [M+H]⁺: 702.0

### Step 5: 5-amino-N-[3-chloro-4-[4-(piperazine-1-carbonyl)piperazine-1-carbonyl]phenyl]-1-[2,3-difluoro-4-(methylamino)phenyl]imidazole-4-carboxamide; 1:1 2,2,2-trifluoroacetic acid

A solution of tert-butyl 4-[4-[4-[[5-amino-1-[2,3-difluoro-4-(methylamino)phenyl]imidazole-4-carbonyl]amino]-2-chlorobenzoyl]piperazine-1-carbonyl]piperazine-1-carboxylate (72.0 mg, 0.1 mmol, 1.0 eq) in methylene chloride (3 mL) was added trifluoroacetic acid (1 mL). The reaction was stirred at 25° C. for 2 h. The reaction was concentrated to dryness to afford a crude product which was purified by Prep-HPLC (Gemini-C18 150×21.2 mm, 5 μm, MeCN-H₂O (0.1% TFA), 15-40) to afford 5-amino-N-[3-chloro-4-[4-(piperazine-1-carbonyl)piperazine-1-carbonyl]phenyl]-1-[2,3-difluoro-4-(methylamino)phenyl]imidazole-4-carboxamide; 1:1 2,2,2-trifluoroacetic acid (19.7 mg, 0.03 mmol, 30.9% yield). MS [M+H]⁺: 602.0

### Compound 187

### 5-amino-N-[4-[4-[(2S)-azetidine-2-carbonyl]piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid

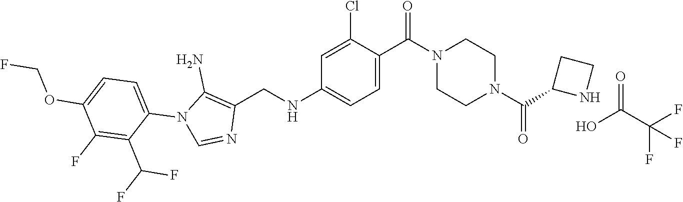

### Step 1: 5-amino-1-[2-(difluoromethyl)-3-fluoro-4-(fluoromethoxy)phenyl]imidazole-4-carbonyl chloride

A solution of 5-amino-1-[2-(difluoromethyl)-3-fluoro-4-(fluoromethoxy)phenyl]imidazole-4-carboxylic acid (570.0 mg, 1.79 mmol, 1.0 eq) in thionyl chloride (10.0 mL, 137.85 mmol, 77.2 eq) was stirred at 25° C. for 1 h. The resulting solution was concentrated under reduced pressure to give 5-amino-1-[2-(difluoromethyl)-3-fluoro-4-(fluoromethoxy)phenyl]imidazole-4-carbonyl chloride (570.0 mg, 1.69 mmol, 89.8% yield). MS [(M−Cl+OCH₃)+H]⁺: 344.0

### Step 2: tert-butyl 4-[4-[[5-amino-1-[2-(difluoromethyl)-3-fluoro-4-(fluoromethoxy)phenyl]imidazole-4-carbonyl]amino]-2-chlorobenzoyl]piperazine-1-carboxylate

To a solution of tert-butyl 4-(4-amino-2-chlorobenzoyl)piperazine-1-carboxylate (401.55 mg, 1.18 mmol, 0.7 eq) and pyridine (2.73 mL, 33.76 mmol, 20.0 eq) in methylene chloride (1 mL) was added a suspension of 5-amino-1-[2-(difluoromethyl)-3-fluoro-4-(fluoromethoxy)phenyl]imidazole-4-carbonyl chloride (570.0 mg, 1.69 mmol, 1.0 eq) in methylene chloride (1 mL) at 25° C. The mixture was stirred at 25° C. for 1 h under N₂. The resulting solution was evaporated under reduced pressure. The residue was purified by column chromatography (silica gel, 0 to 10% MeOH in methylene chloride) to give tert-butyl4-[4-[[5-amino-1-[2-(difluoromethyl)-3-fluoro-4-(fluoromethoxy)phenyl]imidazole-4-carbonyl]amino]-2-chlorobenzoyl]piperazine-1-carboxylate (680.0 mg, 1.06 mmol, 62.8% yield). MS [M+H]⁺: 641.3.

### Step 3: 5-amino-N-[3-chloro-4-(piperazine-1-carbonyl)phenyl]-1-[2-(difluoromethyl)-3-fluoro-4-(fluoromethoxy)phenyl]imidazole-4-carboxamide

A solution of tert-butyl 4-[4-[[5-amino-1-[2-(difluoromethyl)-3-fluoro-4-(fluoromethoxy)phenyl]imidazole-4-carbonyl]amino]-2-chlorobenzoyl]piperazine-1-carboxylate (680.0 mg, 1.06 mmol, 1.0 eq) and hydrogen chloride solution (4.77 mL, 19.1 mmol, 18.0 eq) in methylene chloride (2 mL) was stirred at 25° C. for 1 h. The resulting solution was concentrated under reduced pressure to give 5-amino-N-[3-chloro-4-(piperazine-1-carbonyl)phenyl]-1-[2-(difluoromethyl)-3-fluoro-4-(fluoromethoxy)phenyl]imidazole-4-carboxamide (560.0 mg, 1.04 mmol, 97.6% yield). MS [M+H]⁺: 541.0

### Step 4: tert-butyl(2S)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidine-1-carboxylate

To a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide (50.0 mg, 0.09 mmol, 1.0 eq) in methylene chloride (2 mL) were added (2S)-1-tert-butoxycarbonylazetidine-2-carboxylic acid (20.46 mg, 0.1 mmol, 1.1 eq), 1,3,5,2,4,6-trioxatriphosphorinane (49.95 mg, 0.14 mmol, 1.5 eq) and N,N-diisopropylethylamine (35.84 mg, 0.28 mmol, 3.0 eq). The resulting mixture was stirred at 25° C. for 1 h. The solvent was removed under reduced pressure. The residue was diluted with EtOAc (100 mL), washed with H₂O (4×60 mL), dried over Na₂SO₄ and concentrated under reduced pressure and the residue was purified by flash column chromatography (4:1 petroleum ether:EtOAc) to give tert-butyl(2S)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidine-1-carboxylate (60.0 mg, 0.08 mmol, 89.6% yield). MS [M+H]⁺: 724.1.

### Step 5: 5-amino-N-[4-[4-[(2S)-azetidine-2-carbonyl]piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid

A solution of tert-butyl(2S)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidine-1-carboxylate (50.0 mg, 0.07 mmol, 1.0 eq) and trifluoroacetic acid (1.0 mL) in methylene chloride (1 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15-40) to afford 5-amino-N-[4-[4-[(2S)-azetidine-2-carbonyl]piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid (38.5 mg, 0.05 mmol, 75.0% yield). MS [M+H]⁺: 624.3.

### Compound 188

### 5-amino-N-[4-[4-(azetidine-3-carbonyl)piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid

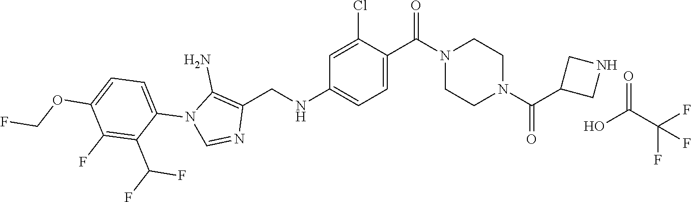

### Step 1: tert-butyl 3-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidine-1-carboxylate

To a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide (70.0 mg, 0.13 mmol, 1.0 eq) in methylene chloride (3 mL) were added 1-BOC-azetidine-3-carboxylic acid (31.25 mg, 0.16 mmol, 1.2 eq), propylphosphonic anhydride (164.71 mg, 0.26 mmol, 2.0 eq) and N,N-diisopropylethylamine (0.09 mL, 0.52 mmol, 4.0 eq). The resulting mixture was stirred at 25° C. for 1 h. The solvent was removed under reduced pressure. The residue was diluted with EtOAc (5 mL), washed with H₂O (3×5 mL), dried over Na₂SO₄ and concentrated under reduced pressure to give tert-butyl 3-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidine-1-carboxylate (75.0 mg, 0.1 mmol, 80.0% yield). MS [M−Boc+H]⁺: 624.1

### Step 2: 5-amino-N-[4-[4-(azetidine-3-carbonyl)piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid

A solution of tert-butyl 3-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidine-1-carboxylate (75.0 mg, 0.1 mmol, 1.0 eq), trifluoroacetic acid (1 mL) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN/H₂O (0.1% TFA), 20%-40%) to afford 5-amino-N-[4-[4-(azetidine-3-carbonyl)piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid (49.2 mg, 0.07 mmol, 64.3% yield). MS [M+H]⁺: 624.1

### Compound 189

### 5-amino-N-[4-[4-[3-(azetidin-3-yloxy) azetidine-1-carbonyl]piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid

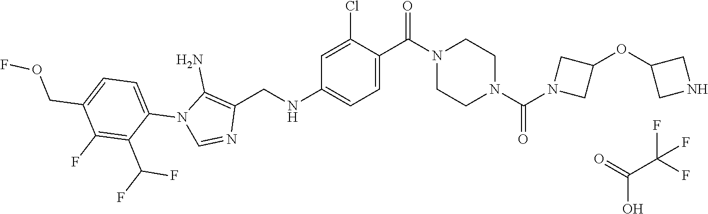

### Step 1: tert-butyl 3-[1-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidin-3-yl]oxyazetidine-1-carboxylate

A solution of tert-butyl 3-(azetidin-3-yloxy) azetidine-1-carboxylate (35.45 mg, 0.16 mmol, 1.4 eq) and triphosgene (16.46 mg, 0.06 mmol, 0.5 eq) in methylene chloride (5 mL) was stirred at 25° C. for 0.5 h. Next, 5-amino-N-[3-chloro-4-(piperazine-1-carbonyl)phenyl]-1-[2-(difluoromethyl)-3-fluoro-4-(fluoromethoxy)phenyl]imidazole-4-carboxamide (60.0 mg, 0.11 mmol, 1.0 eq) and triethylamine (0.15 mL, 1.11 mmol, 10.0 eq) were added, the mixture was stirred at 25° C. for 1 h. Diluted with EA (30 mL) and washed with H₂O (3×15 mL), dried over Na₂SO₄ and concentrated under vacuo and the residue was purified by flash column chromatography eluting with 87:13 methylene chloride:MeOH to give tert-butyl 3-[1-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidin-3-yl]oxyazetidine-1-carboxylate (71.0 mg, 0.09 mmol, 80.5% yield). MS [(M−Boc)+H]⁺: 695.0

### Step 2: 5-amino-N-[4-[4-[3-(azetidin-3-yloxy) azetidine-1-carbonyl]piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid

A solution of tert-butyl 3-[1-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidin-3-yl]oxyazetidine-1-carboxylate (60.0 mg, 0.08 mmol, 1.0 eq) and trifluoroacetic acid (8.6 mg, 0.08 mmol, 1.0 eq) in methylene chloride (1 mL) was stirred at 25° C. for 1 h. The resulting solution was concentrated under reduced pressure. The crude product was purified by Prep-HPLC (Gemini-C18 150×21.2 mm, 5 μm, MeCN-H₂O (0.1% TFA), 15-45) to give 5-amino-N-[4-[4-[3-(azetidin-3-yloxy) azetidine-1-carbonyl]piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid (12.8 mg, 0.02 mmol, 24.0% yield). MS [M+H]⁺: 694.9.

### Compound 190

### 5-amino-N-[4-[4-[3-(azetidin-3-yloxy) azetidine-1-carbonyl]piperazine-1-carbonyl]-3-chlorophenyl]-1-[2,3-difluoro-4-(fluoromethoxy)phenyl]imidazole-4-carboxamide;2,2,2-trifluoroacetic acid

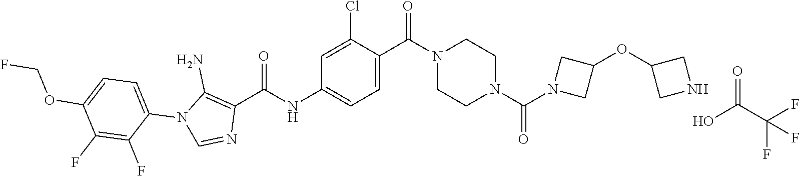

### Step 1: benzyl 3-(1-tert-butoxycarbonylazetidin-3-yl)oxyazetidine-1-carboxylate

A mixture of 1-N-CBZ-3-hydroxyazetidine (15.0 g, 72.38 mmol, 1.0 eq), 1-BOC-3-iodoazetidine (24.59 g, 86.86 mmol, 1.2 eq), and cesium carbonate (70.75 g, 217.2 mmol, 3.0 eq) in DMF (80 mL) was degassed and purged with N₂ three times. Then the mixture was stirred at 100° C. for 24 h under N₂ atmosphere. Afer this time the reaction mixture was concentrated to give a residue. The residue was purified by flash chromatography on silica gel [70:30 petroleum ether:EtOAc] to give benzyl 3-(1-tert-butoxycarbonylazetidin-3-yl)oxyazetidine-1-carboxylate (1850.0 mg, 5.1 mmol, 6.8% yield). MS [M+Na]⁺: 385.0

### Step 2: tert-butyl 3-(azetidin-3-yloxy) azetidine-1-carboxylate

To a solution of benzyl 3-(1-tert-butoxycarbonylazetidin-3-yl)oxyazetidine-1-carboxylate (1850.0 mg, 5.1 mmol, 1.0 eq) in methanol (120 mL) was added palladium (carbon-supported, spherical particles)(430.0 mg, 0.16 mmol, 0.03 eq) under hydrogen atmosphere (balloon). The resulting mixture was stirred at 25° C. for 20 h. The solid was filtered off and solvent was removed under reduced pressure to give tert-butyl 3-(azetidin-3-yloxy) azetidine-1-carboxylate (1100.0 mg, 4.82 mmol, 94.4% yield). MS [M+H]⁺: 229.1

### Step 3: tert-butyl 3-[1-[4-[4-[[5-amino-1-[2,3-difluoro-4-(fluoromethoxy)phenyl]imidazole-4-carbonyl]amino]-2-chlorobenzoyl]piperazine-1-carbonyl]azetidin-3-yl]oxyazetidine-1-carboxylate

To a solution of tert-butyl 3-(azetidin-3-yloxy) azetidine-1-carboxylate (62.8 mg, 0.28 mmol, 1.4 eq) in methylene chloride (1 mL) were added triphosgene (40.82 mg, 0.14 mmol, 0.7 eq) and tert-butyl 3-(azetidin-3-yloxy) azetidine-1-carboxylate (62.8 mg, 0.28 mmol, 1.4 eq). The resulting mixture was stirred at 25° C. for 0.5 h, and then triethylamine (0.14 mL, 0.98 mmol, 5.0 eq) and a solution of 5-amino-N-[3-chloro-4-(piperazine-1-carbonyl)phenyl]-1-[2,3-difluoro-4-(fluoromethoxy)phenyl]imidazole-4-carboxamide (100.0 mg, 0.2 mmol, 1.0 eq) in methylene chloride (1 mL) was added in order. The resulting mixture was stirred at 25° C. for another 1.5 h. After this time, the reaction mixture was diluted with methylene chloride (30 mL). This solution was washed with H₂O (3×15 mL), dried over Na₂SO₄ and concentrated under reduced pressure. The residue was purified by flash column chromatography (methylene chloride:MeOH=96:4) to give tert-butyl 3-[1-[4-[4-[[5-amino-1-[2,3-difluoro-4-(fluoromethoxy)phenyl]imidazole-4-carbonyl]amino]-2-chlorobenzoyl]piperazine-1-carbonyl]azetidin-3-yl]oxyazetidine-1-carboxylate (115.0 mg, 0.15 mmol, 76.7% yield). MS [M+Na]⁺: 784.9

### Step 4: 5-amino-N-[4-[4-[3-(azetidin-3-yloxy) azetidine-1-carbonyl]piperazine-1-carbonyl]-3-chlorophenyl]-1-[2,3-difluoro-4-(fluoromethoxy)phenyl]imidazole-4-carboxamide;2,2,2-trifluoroacetic acid

To a solution of tert-butyl 3-[1-[4-[4-[[5-amino-1-[2,3-difluoro-4-(fluoromethoxy)phenyl]imidazole-4-carbonyl]amino]-2-chlorobenzoyl]piperazine-1-carbonyl]azetidin-3-yl]oxyazetidine-1-carboxylate (115.0 mg, 0.15 mmol, 1.0 eq) in methylene chloride (1 mL) was added trifluoroacetic acid (0.5 mL, 6.53 mmol, 43.33 eq). The resulting mixture was stirred at 25° C. for 1 h. After concentration under reduced pressure, the residue was purified by prep-HPLC [Gemini-C18 150×21.2 mm, 5 μm, MeCN— H₂O (0.1% TFA), 5%-50%] to give 5-amino-N-[4-[4-[3-(azetidin-3-yloxy) azetidine-1-carbonyl]piperazine-1-carbonyl]-3-chlorophenyl]-1-[2,3-difluoro-4-(fluoromethoxy)phenyl]imidazole-4-carboxamide; 1:1 2,2,2-trifluoroacetic acid (20.8 mg, 0.03 mmol, 17.0% yield). MS [M+H]⁺: 663.0

### Compound 191

### 5-amino-N-[4-[4-[3-(azetidin-3-yl) azetidine-1-carbonyl]piperazine-1-carbonyl]-3-chlorophenyl]-1-[2,3-difluoro-4-(fluoromethoxy)phenyl]imidazole-4-carboxamide; 1:1 2,2,2-trifluoroacetic acid

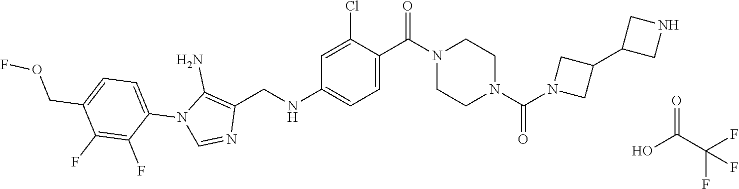 [SMILES: Nc1c(CNc2ccc(C(=O)N3CCN(C(=O)N4CC(C5CNC5)C4)CC3)c(Cl)c2)ncn1-c1ccc(COF)c(F)c1F ; O=C(O)C(F)(F)F]

### Step 1: tert-butyl 4-[4-[[5-amino-1-[2,3-difluoro-4-(fluoromethoxy)phenyl]imidazole-4-carbonyl]amino]-2-chlorobenzoyl]piperazine-1-carboxylate

To a solution of tert-butyl 4-(4-amino-2-chlorobenzoyl)piperazine-1-carboxylate (233.48 mg, 0.69 mmol, 0.7 eq) and pyridine (1.59 mL, 19.6 mmol, 20.0 eq) was added a suspension of 5-amino-1-[2,3-difluoro-4-(fluoromethoxy)phenyl]imidazole-4-carbonyl chloride (300.0 mg, 0.98 mmol, 1.0 eq) in methylene chloride (1 mL). The mixture was stirred at 25° C. for 1 h under N₂. The resulting solution was evaporated under reduced pressure. The residue was purified by column chromatography (silica gel, 0 to 10% MeOH in methylene chloride) to give tert-butyl 4-[4-[[5-amino-1-[2,3-difluoro-4-(fluoromethoxy)phenyl]imidazole-4-carbonyl]amino]-2-chlorobenzoyl]piperazine-1-carboxylate (326.0 mg, 0.54 mmol, 47.4% yield). MS [M+H]⁺: 609.1.

### Step 2: 5-amino-N-[3-chloro-4-(piperazine-1-carbonyl)phenyl]-1-[2,3-difluoro-4-(fluoromethoxy)phenyl]imidazole-4-carboxamide

A solution of tert-butyl 4-[4-[[5-amino-1-[2,3-difluoro-4-(fluoromethoxy)phenyl]imidazole-4-carbonyl]amino]-2-chlorobenzoyl]piperazine-1-carboxylate (326.0 mg, 0.54 mmol, 1.0 eq) and hydrogen chloride solution (2.51 mL, 10.0 mmol, 18.7 eq, 1M) in methylene chloride (5 mL) was stirred at 25° C. for 1 h. The reaction mixture was then concentrated under reduced pressure at 30° C. The resulting solution was evaporated to dryness and azeotroped with methylene chloride to give the title compound 5-amino-N-[3-chloro-4-(piperazine-1-carbonyl)phenyl]-1-[2,3-difluoro-4-(fluoromethoxy)phenyl]imidazole-4-carboxamide (250.0 mg, 0.49 mmol, 91.8% yield), which was used without further purification. MS [M+H]⁺: 509.2

### Step 3: tert-butyl 3-[1-[4-[4-[[5-amino-1-[2,3-difluoro-4-(fluoromethoxy)phenyl]imidazole-4-carbonyl]amino]-2-chlorobenzoyl]piperazine-1-carbonyl]azetidin-3-yl]azetidine-1-carboxylate

A solution of 5-amino-N-[3-chloro-4-(piperazine-1-carbonyl)phenyl]-1-[2,3-difluoro-4-(fluoromethoxy)phenyl]imidazole-4-carboxamide (100.0 mg, 0.2 mmol, 1.0 eq) and triphosgene (29.16 mg, 0.1 mmol, 0.5 eq) in methylene chloride (5 mL) was stirred at 25° C. for 0.5 h. Next, tert-butyl 3-(azetidin-3-yl) azetidine-1-carboxylate (125.15 mg, 0.59 mmol, 3.0 eq) and triethylamine (0.27 mL, 1.97 mmol, 10.0 eq) were added, and the solution was stirred at 25° C. for 1 h. Diluted with EtOAc (40 mL) and washed with H₂O (3×20 mL), dried over Na₂SO₄ and concentrated under vacuo and the residue was purified by flash column chromatography eluting with 87:13 methylene chloride:MeOH to give tert-butyl 3-[1-[4-[4-[[5-amino-1-[2,3-difluoro-4-(fluoromethoxy)phenyl]imidazole-4-carbonyl]amino]-2-chlorobenzoyl]piperazine-1-carbonyl]azetidin-3-yl]azetidine-1-carboxylate (60.0 mg, 0.08 mmol, 40.9% yield). MS [(M−Boc)+H]⁺: 647.0

### Step 4: 5-amino-N-[4-[4-[3-(azetidin-3-yl) azetidine-1-carbonyl]piperazine-1-carbonyl]-3-chlorophenyl]-1-[2,3-difluoro-4-(fluoromethoxy)phenyl]imidazole-4-carboxamide; 1:1 2,2,2-trifluoroacetic acid

A solution of tert-butyl 3-[1-[4-[4-[[5-amino-1-[2,3-difluoro-4-(fluoromethoxy)phenyl]imidazole-4-carbonyl]amino]-2-chlorobenzoyl]piperazine-1-carbonyl]azetidin-3-yl]azetidine-1-carboxylate (50.0 mg, 0.07 mmol, 1.0 eq) and trifluoroacetic acid (7.63 mg, 0.07 mmol, 1.0 eq) in methylene chloride (1 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 15%-40%) to yield 5-amino-N-[4-[4-[3-(azetidin-3-yl) azetidine-1-carbonyl]piperazine-1-carbonyl]-3-chlorophenyl]-1-[2,3-difluoro-4-(fluoromethoxy)phenyl]imidazole-4-carboxamide; 1:1 2,2,2-trifluoroacetic acid (12.3 mg, 0.02 mmol, 27.3% yield). MS [M+H]⁺: 647.1.

### Compound 192

### 5-amino-N-[4-[4-[(1S,5R,6r)-3-azabicyclo[3.1.0]hexane-6-carbonyl]piperazine-1-carbonyl]-3-fluoro-phenyl]-1-[1-(2-chloroallyl)-3-(trifluoromethyl)pyrazol-4-yl]imidazole-4-carboxamide; 1:1 2,2,2-trifluoroacetic acid

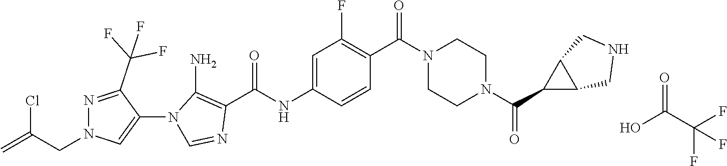 [SMILES: C=C(Cl)Cn1cc(-n2cnc(C(=O)Nc3ccc(C(=O)N4CCN(C(=O)[C@H]5[C@@H]6CNC[C@@H]65)CC4)c(F)c3)c2N)c(C(F)(F)F)n1 ; O=C(O)C(F)(F)F]

### Step 1: tert-butyl (1S,5R)-6-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate

A solution of tert-butyl (1S,5R)-6-[4-[4-[[5-amino-1-[3-(trifluoromethyl)-1H-pyrazol-4-yl]imidazole-4-carbonyl]amino]-2-fluorobenzoyl]piperazine-1-carbonyl]-3-azabicyclo[3.1.0]hexane-3-carboxylate (50.0 mg, 0.07 mmol, 1.0 eq), 2-chloroallyl 4-methylbenzenesulfonate (27.39 mg, 0.11 mmol, 1.5 eq) and potassium carbonate (30.68 mg, 0.22 mmol, 3.0 eq) in DMF (2 mL) was stirred at 25° C. for 2 h. The resulting solution was diluted with 5 ml of water and then extracted with 3×5 mL EtOAc. The organic layers were combined, dried and concentrated under reduced pressure. The residue was applied on a silica gel column and eluted with 10:1 methylene chloride:MeOH to give tert-butyl (1S,5R)-6-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate (40.0 mg, 0.05 mmol, 72.0% yield). MS [M+H]⁺: 750.1

### Step 2: 5-amino-N-[4-[4-[(1S,5R)-3-azabicyclo[3.1.0]hexane-6-carbonyl]piperazine-1-carbonyl]-3-fluorophenyl]-1-[1-(2-chloroallyl)-3-(trifluoromethyl)pyrazol-4-yl]imidazole-4-carboxamide; 1:1 2,2,2-trifluoroacetic acid

A solution of tert-butyl (1S,5R)-6-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate (40.0 mg, 0.05 mmol, 1.0 eq), trifluoroacetic acid (0.5 mL, 6.4 mmol, 120 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 20-40%) to give 5-amino-N-[4-[4-[(1S,5R)-3-azabicyclo[3.1.0]hexane-6-carbonyl]piperazine-1-carbonyl]-3-fluorophenyl]-1-[1-(2-chloroallyl)-3-(trifluoromethyl)pyrazol-4-yl]imidazole-4-carboxamide; 1:1 2,2,2-trifluoroacetic acid (10.8 mg, 0.01 mmol, 26.2% yield). MS [M+H]⁺: 650.1

### Compound 193

### 5-amino-N-[4-[4-[(1S,5R,6r)-3-azabicyclo[3.1.0]hexane-6-carbonyl]piperazine-1-carbonyl]-3-fluorophenyl]-1-[1-prop-2-ynyl-3-(trifluoromethyl)pyrazol-4-yl]imidazole-4-carboxamide; 1:1 2,2,2-trifluoroacetic acid

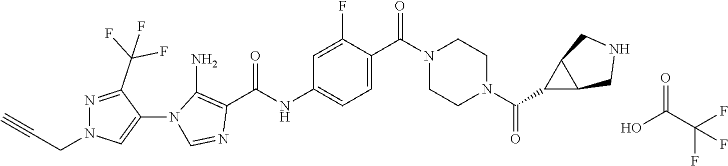

### Step 1: 5-amino-1-[1-[(4-methoxyphenyl)methyl]-3-(trifluoromethyl)pyrazol-4-yl]imidazole-4-carbonyl chloride

A solution of 5-amino-1-[1-[(4-methoxyphenyl)methyl]-3-(trifluoromethyl)pyrazol-4-yl]imidazole-4-carboxylic acid (200.0 mg, 0.52 mmol, 1.0 eq) in thionyl chloride (3.0 mL, 41.35 mmol, 78.85 eq) was stirred at 25° C. for 1 h. The resulting solution was concentrated under reduced pressure to give 5-amino-1-[1-[(4-methoxyphenyl)methyl]-3-(trifluoromethyl)pyrazol-4-yl]imidazole-4-carbonyl chloride (200.0 mg, 0.5 mmol, 95.4% yield). MS [M−Cl+OMe+H]⁺: 396.0

### Step 2: tert-butyl (1S,5R)-6-[4-[4-[[5-amino-1-[1-[(4-methoxyphenyl)methyl]-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate

A solution of tert-butyl (1S,5R)-6-[4-(4-amino-2-fluorobenzoyl)piperazine-1-carbonyl]-3-azabicyclo[3.1.0]hexane-3-carboxylate (216.38 mg, 0.5 mmol, 1.0 eq) and pyridine (0.81 mL, 10.01 mmol, 20.0 eq) in methylene chloride (2 mL) was stirred at 25° C. Next, a solution of 5-amino-1-[1-[(4-methoxyphenyl)methyl]-3-(trifluoromethyl)pyrazol-4-yl]imidazole-4-carbonyl chloride (200.0 mg, 0.5 mmol, 1.0 eq) in methylene chloride (2 mL) was added, and the mixture was stirred at 25° C. for 1 h under N₂. The resulting solution was concentrated under reduced pressure. The residue was applied on a silica gel column and eluted with 10:1 methylene chloride:MeOH to give tert-butyl (1S,5R)-6-[4-[4-[[5-amino-1-[1-[(4-methoxyphenyl)methyl]-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate (320.0 mg, 0.4 mmol, 65.9% yield). MS [M+H]⁺: 796.2

### Step 3: 5-amino-N-[4-[4-[(1R,5S)-3-azabicyclo[3.1.0]hexane-6-carbonyl]piperazine-1-carbonyl]-3-fluorophenyl]-1-[3-(trifluoromethyl)-1H-pyrazol-4-yl]imidazole-4-carboxamide

A solution of tert-butyl (1R,5S)-6-[4-[4-[[5-amino-1-[1-[(4-methoxyphenyl)methyl]-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate (320.0 mg, 0.4 mmol, 1.0 eq) in trifluoroacetic acid (5.0 mL, 64.9 mmol, 161.39 eq) was stirred at 70° C. for 8 h. The reaction was concentrated to dryness to give 5-amino-N-[4-[4-[(1R,5S)-3-azabicyclo[3.1.0]hexane-6-carbonyl]piperazine-1-carbonyl]-3-fluorophenyl]-1-[3-(trifluoromethyl)-1H-pyrazol-4-yl]imidazole-4-carboxamide (180.0 mg, 0.31 mmol, 49.0% yield). MS [M+H]⁺: 576.3

### Step 4: tert-butyl (1R,5S)-6-[4-[4-[[5-amino-1-[3-(trifluoromethyl)-1H-pyrazol-4-yl]imidazole-4-carbonyl]amino]-2-fluorobenzoyl]piperazine-1-carbonyl]-3-azabicyclo[3.1.0]hexane-3-carboxylate

To a solution of 5-amino-N-[4-[4-[(1R,5S)-3-azabicyclo[3.1.0]hexane-6-carbonyl]piperazine-1-carbonyl]-3-fluorophenyl]-1-[3-(trifluoromethyl)-1H-pyrazol-4-yl]imidazole-4-carboxamide (180.0 mg, 0.31 mmol, 1.0 eq) in the THF (5 mL) and water (1 mL) was added tert-butoxy carbonyl anhydride (68.26 mg, 0.31 mmol, 1.0 eq) and sodium hydrogen carbonate (78.82 mg, 0.94 mmol, 3.0 eq). The mixture was stirred at 25° C. for 1 h. The resulting solution was diluted with 10 ml of water, then extracted with 3×10 mL of ethyl acetate. The organic layers were combined, concentrated to dryness to afford crude product. The crude was then purified by flash column chromatography eluting with 10:1 methylene chloride:MeOH to give tert-butyl (1R,5S)-6-[4-[4-[[5-amino-1-[3-(trifluoromethyl)-1H-pyrazol-4-yl]imidazole-4-carbonyl]amino]-2-fluorobenzoyl]piperazine-1-carbonyl]-3-azabicyclo[3.1.0]hexane-3-carboxylate (140.0 mg, 0.21 mmol, 66.5% yield). MS [M−tBu+H]⁺: 620.2

### Step 5: tert-butyl (1S,5R)-6-[4-[4-[[5-amino-1-[1-prop-2-ynyl-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate

To a solution of tert-butyl (1S,5R)-6-[4-[4-[[5-amino-1-[3-(trifluoromethyl)-1H-pyrazol-4-yl]imidazole-4-carbonyl]amino]-2-fluorobenzoyl]piperazine-1-carbonyl]-3-azabicyclo[3.1.0]hexane-3-carboxylate (115.0 mg, 0.17 mmol, 1.0 eq) in DMF (3 mL) was added propargyl bromide (30.37 mg, 0.26 mmol, 1.5 eq) and potassium carbonate (70.57 mg, 0.51 mmol, 3.0 eq), and the mixture was stirred at 25° C. for 2 h. The resulting solution was diluted with 5 ml of water then extracted with 3×5 mL of ethyl acetate. The organic layers were combined, concentrated to dryness to afford crude product. The crude was then purified by flash column chromatography eluting with 10:1 methylene chloride:MeOH to give tert-butyl (1S,5R)-6-[4-[4-[[5-amino-1-[1-prop-2-ynyl-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate (90.0 mg, 0.13 mmol, 74.0% yield). MS [M−t−Bu+H]⁺: 658.2

### Step 6: 5-amino-N-[4-[4-[(1S,5R)-3-azabicyclo[3.1.0]hexane-6-carbonyl]piperazine-1-carbonyl]-3-fluorophenyl]-1-[1-prop-2-ynyl-3-(trifluoromethyl)pyrazol-4-yl]imidazole-4-carboxamide; 1:1 2,2,2-trifluoroacetic acid

A solution of tert-butyl (1S,5R)-6-[4-[4-[[5-amino-1-[1-prop-2-ynyl-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate (90.0 mg, 0.13 mmol, 1.0 eq), trifluoroacetic acid (1.0 mL, 12.98 mmol, 102.93 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 20-40) to afford 5-amino-N-[4-[4-[(1S,5R)-3-azabicyclo[3.1.0]hexane-6-carbonyl]piperazine-1-carbonyl]-3-fluorophenyl]-1-[1-prop-2-ynyl-3-(trifluoromethyl)pyrazol-4-yl]imidazole-4-carboxamide; 1:1 2,2,2-trifluoroacetic acid (44.1 mg, 0.06 mmol, 56.0% yield). MS [M+H]⁺: 614.1

### Compound 194

### 5-amino-N-[4-[4-[(1S,5R,6r)-3-azabicyclo[3.1.0]hexane-6-carbonyl]piperazine-1-carbonyl]-3-fluorophenyl]-1-[1-(2-chloro-2,2-difluoroethyl)-3-(trifluoromethyl)pyrazol-4-yl]imidazole-4-carboxamide; 1:1 2,2,2-trifluoroacetic acid

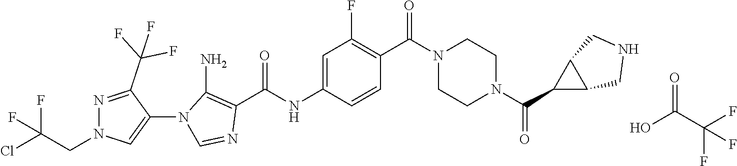 [SMILES: Nc1c(C(=O)Nc2ccc(C(=O)N3CCN(C(=O)[C@H]4[C@@H]5CNC[C@@H]54)CC3)c(F)c2)ncn1-c1cn(CC(F)(F)Cl)nc1C(F)(F)F ; O=C(O)C(F)(F)F]

### Step 1:(2-chloro-2,2-difluoroethyl) trifluoromethanesulfonate

A solution of 2-chloro-2,2-difluoroethanol (860.0 mg, 7.38 mmol, 1.0 eq), triethylamine (1491.29 mg, 14.77 mmol, 2.0 eq), 4-dimethylaminopyridine (180.38 mg, 1.48 mmol, 0.2 eq) and trifluoromethanesulfonyl chloride (1866.06 mg, 11.07 mmol, 1.5 eq) in methylene chloride (5 mL) was stirred at 25° C. for 1 h. The resulting solution was diluted with 5 ml of water, then extracted with 2×5 mL of methylene chloride. The organic layers were combined and concentrated under reduced pressure to give (2-chloro-2,2-difluoroethyl) trifluoromethanesulfonate (1150.0 mg, 4.63 mmol, 62.7% yield).

### Step 2: tert-butyl (1S,5R)-6-[4-[4-[[5-amino-1-[1-(2-chloro-2,2-difluoroethyl)-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate

A solution of tert-butyl (1S,5R)-6-[4-[4-[[5-amino-1-[3-(trifluoromethyl)-1H-pyrazol-4-yl]imidazole-4-carbonyl]amino]-2-fluorobenzoyl]piperazine-1-carbonyl]-3-azabicyclo[3.1.0]hexane-3-carboxylate (100.0 mg, 0.15 mmol, 1.0 eq), (2-chloro-2,2-difluoroethyl)trifluoromethanesulfonate (55.18 mg, 0.22 mmol, 1.5 eq) and potassium carbonate (61.37 mg, 0.44 mmol, 3.0 eq) in DMF (3 mL) was stirred at 25° C. for 2 h. The resulting solution was diluted with 10 ml of water then extracted with 3×10 mL of EtOAc. The organic layers were combined, dried and concentrated under reduced pressure. The residue was applied on a silica gel column and eluted with 10:1 methylene chloride:MeOH to give tert-butyl (1S,5R)-6-[4-[4-[[5-amino-1-[1-(2-chloro-2,2-difluoroethyl)-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate (75.0 mg, 0.1 mmol, 65.5% yield). MS [M−t−Bu+H]⁺: 718.1

### Step 3: 5-amino-N-[4-[4-[(1S,5R)-3-azabicyclo[3.1.0]hexane-6-carbonyl]piperazine-1-carbonyl]-3-fluorophenyl]-1-[1-(2-chloro-2,2-difluoroethyl)-3-(trifluoromethyl)pyrazol-4-yl]imidazole-4-carboxamide; 1:1 2,2,2-trifluoroacetic acid

A solution of tert-butyl (1S,5R)-6-[4-[4-[[5-amino-1-[1-(2-chloro-2,2-difluoroethyl)-3-(trifluoromethyl)pyrazol-4-yl]imidazole-4-carbonyl]amino]-2-fluorobenzoyl]piperazine-1-carbonyl]-3-azabicyclo[3.1.0]hexane-3-carboxylate (75.0 mg, 0.1 mmol, 1.0 eq), trifluoroacetic acid (0.91 mL, 11.76 mmol, 121.38 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm MeCN-H₂O (0.1% TFA), 20-40) to give 5-amino-N-[4-[4-[(1S,5R)-3-azabicyclo[3.1.0]hexane-6-carbonyl]piperazine-1-carbonyl]-3-fluorophenyl]-1-[1-(2-chloro-2,2-difluoroethyl)-3-(trifluoromethyl)pyrazol-4-yl]imidazole-4-carboxamide; 1:1 2,2,2-trifluoroacetic acid (33.2 mg, 0.04 mmol, 43.5% yield). MS [M+H]⁺: 674.2

### Compound 195

### 5-amino-1-[1-(2-chloroallyl)-3-(trifluoromethyl)pyrazol-4-yl]-N-[3-fluoro-4-[4-(piperazine-1-carbonyl)piperazine-1-carbonyl]phenyl]imidazole-4-carboxamide; 1:1 2,2,2-trifluoroacetic acid

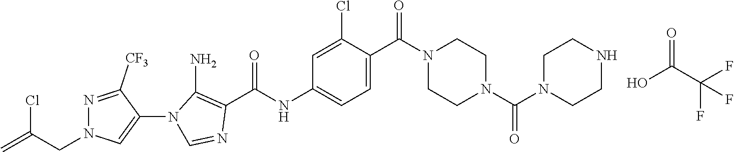

### Step 1: 5-amino-1-[1-[(4-methoxyphenyl)methyl]-3-(trifluoromethyl)pyrazol-4-yl]imidazole-4-carbonyl chloride

A solution of 5-amino-1-[1-[(4-methoxyphenyl)methyl]-3-(trifluoromethyl)pyrazol-4-yl]imidazole-4-carboxylic acid (1050.0 mg, 2.75 mmol, 1.0 eq) in thionyl chloride (15.0 mL, 206.54 mmol, 75.01 eq) was stirred under N₂ at 25° C. for 2 h. The solvent was removed under reduced pressure to give 5-amino-1-[1-[(4-methoxyphenyl)methyl]-3-(trifluoromethyl)pyrazol-4-yl]imidazole-4-carbonyl chloride (1100.0 mg, 2.75 mmol, 96.9% yield), the crude product was used directly without further purification. MS [M−Cl+OCH₃+H]⁺: 396.0

### Step 2: tert-butyl 4-[4-[[5-amino-1-[1-[(4-methoxyphenyl)methyl]-3-(trifluoromethyl)pyrazol-4-yl]imidazole-4-carbonyl]amino]-2-fluorobenzoyl]piperazine-1-carboxylate

To a solution of tert-butyl 4-(4-amino-2-fluorobenzoyl)piperazine-1-carboxylate (533.88 mg, 1.65 mmol, 0.6 eq) in methylene chloride (25 mL) was added pyridine (1088.28 mg, 13.76 mmol, 5.0 eq) and 5-amino-1-[1-[(4-methoxyphenyl)methyl]-3-(trifluoromethyl)pyrazol-4-yl]imidazole-4-carbonyl chloride (1100.0 mg, 2.75 mmol, 1.0 eq) under N₂ atmosphere, the resulting mixture was stirred at 25° C. for 2 h. The solution was concentrated under reduced pressure, and the residue was diluted with EtOAc (50 mL), washed with an aqueous solution of citric acid (3×35 mL), dried over Na₂SO₄ and concentrated under reduced pressure to give tert-butyl 4-[4-[[5-amino-1-[1-[(4-methoxyphenyl)methyl]-3-(trifluoromethyl)pyrazol-4-yl]imidazole-4-carbonyl]amino]-2-fluorobenzoyl]piperazine-1-carboxylate (1360.0 mg, 1.98 mmol, 65.5% yield). The crude product was used directly without further purification. MS [M+H]⁺: 686.9

### Step 3: 5-amino-N-[3-fluoro-4-(piperazine-1-carbonyl)phenyl]-1-[3-(trifluoromethyl)-1H-pyrazol-4-yl]imidazole-4-carboxamide

A solution of tert-butyl 4-[4-[[5-amino-1-[1-[(4-methoxyphenyl)methyl]-3-(trifluoromethyl)pyrazol-4-yl]imidazole-4-carbonyl]amino]-2-fluorobenzoyl]piperazine-1-carboxylate (1360.0 mg, 1.8 mmol, 1.0 eq) in trifluoroacetic acid (8.0 mL, 104.47 mmol, 57.97 eq) was stirred at 70° C. for 8 h. After concentration under reduced pressure, the residue was purified by flash column chromatography (90:10 methylene chloride:MeOH) to give 5-amino-N-[3-fluoro-4-(piperazine-1-carbonyl)phenyl]-1-[3-(trifluoromethyl)-1H-pyrazol-4-yl]imidazole-4-carboxamide (710.0 mg, 1.52 mmol, 81.1% yield). MS [M+H]⁺: 467.0

### Step 4: tert-butyl 4-[4-[4-[[5-amino-1-[3-(trifluoromethyl)-1H-pyrazol-4-yl]imidazole-4-carbonyl]amino]-2-fluorobenzoyl]piperazine-1-carbonyl]piperazine-1-carboxylate

To a solution of 1-BOC-piperazine (95.84 mg, 0.51 mmol, 1.2 eq) in methylene chloride (2 mL) were added triphosgene (76.35 mg, 0.26 mmol, 0.6 eq) and triethylamine (0.21 mL, 1.5 mmol, 3.5 eq). The resulting mixture was stirred at 25° C. for 0.5 h. Next triethylamine (0.21 mL, 1.5 mmol, 3.5 eq) and a solution of 5-amino-N-[3-fluoro-4-(piperazine-1-carbonyl)phenyl]-1-[3-(trifluoromethyl)-1H-pyrazol-4-yl]imidazole-4-carboxamide (200.0 mg, 0.43 mmol, 1.0 eq) in methylene chloride (1 mL) were added in order, and the resulting mixture was stirred at 25° C. for another 1.5 h. The mixture was then diluted with methylene chloride (30 mL), washed with H₂O (3×15 mL), dried over Na₂SO₄ and concentrated under reduced pressure. The residue was purified by flash column chromatography (96:4 methylene chloride:MeOH) to give tert-butyl 4-[4-[4-[[5-amino-1-[3-(trifluoromethyl)-1H-pyrazol-4-yl]imidazole-4-carbonyl]amino]-2-fluorobenzoyl]piperazine-1-carbonyl]piperazine-1-carboxylate (192.0 mg, 0.28 mmol, 63.3% yield). MS: [M+H]⁺: 679.0

### Step 5: tert-butyl 4-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]piperazine-1-carboxylate

To a solution of tert-butyl 4-[4-[4-[[5-amino-1-[3-(trifluoromethyl)-1H-pyrazol-4-yl]imidazole-4-carbonyl]amino]-2-fluorobenzoyl]piperazine-1-carbonyl]piperazine-1-carboxylate (90.0 mg, 0.13 mmol, 1.0 eq) in DMF (3 mL) were added potassium carbonate (54.99 mg, 0.4 mmol, 3.0 eq) and 2-chloroallyl 4-methylbenzenesulfonate (49.08 mg, 0.2 mmol, 1.5 eq) in DMF (3 mL). The resulting mixture was stirred at 25° C. for 2 h. The resulting solution was diluted with 30 ml of water then extracted with EtOAc (3×10 mL). The organic layers were combined, dried and concentrated under reduced pressure. The residue was applied on a silica gel column and eluted with 93:7 methylene chloride:MeOH to give tert-butyl 4-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]piperazine-1-carboxylate (58.0 mg, 0.08 mmol, 48.2% yield). MS [M+H]⁺: 753.0

### Step 6: 5-amino-1-[1-(2-chloroallyl)-3-(trifluoromethyl)pyrazol-4-yl]-N-[3-fluoro-4-[4-(piperazine-1-carbonyl)piperazine-1-carbonyl]phenyl]imidazole-4-carboxamide; 1:1 2,2,2-trifluoroacetic acid

To a solution of tert-butyl 4-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]piperazine-1-carboxylate (58.0 mg, 0.06 mmol, 1.0 eq) in methylene chloride (1 mL) was added trifluoroacetic acid (0.5 mL, 6.53 mmol, 102.16 eq) under N₂ atmosphere. The resulting mixture was stirred at 25° C. for 1 h. After concentration under reduced pressure, the residue was purified by prep-HPLC [Gemini-C18 150×21.2 mm, 5 μm, MeCN— H₂O (0.1% TFA), 30-50%] to give 5-amino-1-[1-(2-chloroallyl)-3-(trifluoromethyl)pyrazol-4-yl]-N-[3-fluoro-4-[4-(piperazine-1-carbonyl)piperazine-1-carbonyl]phenyl]imidazole-4-carboxamide; 1:1 2,2,2-trifluoroacetic acid (47.7 mg, 0.06 mmol, 79.1% yield). MS [M+H]⁺: 652.9

### Compound 196

### 5-amino-1-[1-(2-chloroallyl)-3-(trifluoromethyl)pyrazol-4-yl]-N-[3-fluoro-4-[4-(piperazine-1-carbonyl)piperidine-1-carbonyl]phenyl]imidazole-4-carboxamide;2,2,2-trifluoroacetic acid

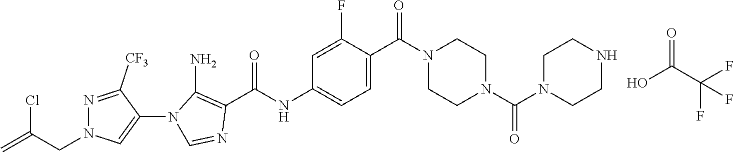

### Step 1: 2-chloroallyl 4-methylbenzenesulfonate

A solution of 2-chloro-2-propen-1-ol (200.0 mg, 2.16 mmol, 1.0 eq), triethylamine (0.6 mL, 4.32 mmol, 2.0 eq), 4-dimethylaminopyridine (52.82 mg, 0.43 mmol, 0.2 eq) and p-toluenesulfonyl chloride (494.55 mg, 2.59 mmol, 1.2 eq) in methylene chloride (3 mL) was stirred at 25° C. for 1 h. The resulting solution was concentrated under reduced pressure. The residue applied on a silica gel column and eluted with 1:2 EtOAc:petroleum ether to give 2-chloroallyl 4-methylbenzenesulfonate (310.0 mg, 1.26 mmol, 58.1% yield). MS [M+H]⁺: 247.0

### Step 2: methyl 1-(2-fluoro-4-nitrobenzoyl)piperidine-4-carboxylate

To a solution of 2-fluoro-4-nitrobenzoic acid (5.0 g, 27.01 mmol, 1.0 eq) in methylene chloride (50 mL) was added methyl 4-piperidinecarboxylate (4.64 g, 32.4 mmol, 1.2 eq), 2-(7-aza-1H-benzotriazole-1-yl)-1,1,3,3-tetramethyluronium hexafluorophosphate (20.54 g, 54.02 mmol, 2.0 eq) and N,N-diisopropylethylamine (14.11 mL, 81.03 mmol, 3.0 eq), and the mixture was stirred at 25° C. for 1 h. The reaction was quenched with water (25 mL). The resulting solution was extracted with methylene chloride (3×20 mL). The combined organic phases were dried over anhydrous sodium sulfate and concentrated under reduced pressure. The residue was purified by flash column to give methyl 1-(2-fluoro-4-nitrobenzoyl)piperidine-4-carboxylate (6.5 g, 21 mmol, 78% yield). MS [M+H]⁺: 311.0

### Step 3: methyl 1-(4-amino-2-fluorobenzoyl)piperidine-4-carboxylate

A solution of methyl 1-(2-fluoro-4-nitrobenzoyl)piperidine-4-carboxylate (6.5 g, 20.95 mmol, 1.0 eq), iron powder (5.87 g, 104.74 mmol, 5.0 eq) and ammonium chloride (5.6 g, 104.74 mmol, 5.0 eq) in ethanol (100 mL) and water (50 mL) was stirred at 50° C. for 3 h. The solid was filtered out. The crude was then purified by flash column chromatography eluting with 40% EtOAc in petroleum ether to give methyl 1-(4-amino-2-fluorobenzoyl)piperidine-4-carboxylate (4.8 g, 17 mmol, 82% yield). MS [M+H]⁺: 281.1

### Step 4: methyl 1-[4-[[5-amino-1-[1-[(4-methoxyphenyl)methyl]-3-(trifluoromethyl)pyrazol-4-yl]imidazole-4-carbonyl]amino]-2-fluorobenzoyl]piperidine-4-carboxylate

A solution of methyl 1-(4-amino-2-fluorobenzoyl)piperidine-4-carboxylate (350.59 mg, 1.25 mmol, 1.0 eq) and pyridine (1.01 mL, 12.51 mmol, 10.0 eq) in methylene chloride (2 mL) was stirred at 25° C. Next 5-amino-1-[1-[(4-methoxyphenyl)methyl]-3-(trifluoromethyl)pyrazol-4-yl]imidazole-4-carbonyl chloride (500.0 mg, 1.25 mmol, 1.0 eq) in methylene chloride (2 mL) was added, and the mixture was stirred at 25° C. for 1 h under N₂. The resulting solution was concentrated under reduced pressure. The residue was applied on a silica gel column and eluted with 10:1 methylene chloride:MeOH to give methyl 1-[4-[[5-amino-1-[1-[(4-methoxyphenyl)methyl]-3-(trifluoromethyl)pyrazol-4-yl]imidazole-4-carbonyl]amino]-2-fluorobenzoyl]piperidine-4-carboxylate (690.0 mg, 1.07 mmol, 85.7% yield). MS [M+H]⁺: 644.1

### Step 5: methyl 1-[4-[[5-amino-1-[3-(trifluoromethyl)-1H-pyrazol-4-yl]imidazole-4-carbonyl]amino]-2-fluorobenzoyl]piperidine-4-carboxylate

A solution of methyl 1-[4-[[5-amino-1-[1-[(4-methoxyphenyl)methyl]-3-(trifluoromethyl)pyrazol-4-yl]imidazole-4-carbonyl]amino]-2-fluorobenzoyl]piperidine-4-carboxylate (690.0 mg, 1.07 mmol, 1.0 eq) in trifluoroacetic acid (10.0 mL) was stirred at 70° C. for 8 h. The reaction was concentrated to dryness to give methyl 1-[4-[[5-amino-1-[3-(trifluoromethyl)-1H-pyrazol-4-yl]imidazole-4-carbonyl]amino]-2-fluorobenzoyl]piperidine-4-carboxylate (480.0 mg, 0.92 mmol, 80.4% yield). MS [M+H]⁺: 524.0

### Step 6: 1-[4-[[5-amino-1-[3-(trifluoromethyl)-1H-pyrazol-4-yl]imidazole-4-carbonyl]amino]-2-fluorobenzoyl]piperidine-4-carboxylic acid

Methyl 1-[4-[[5-amino-1-[3-(trifluoromethyl)-1H-pyrazol-4-yl]imidazole-4-carbonyl]amino]-2-fluorobenzoyl]piperidine-4-carboxylate (480.0 mg, 0.92 mmol, 1.0 eq) was dissolved in isopropyl alcohol (5 mL). A solution of sodium hydroxide (183.39 mg, 4.58 mmol, 5.0 eq) in water (2.5 mL) was added dropwise at 25° C. and stirred at 50° C. for 3 h. The solvent was then evaporated. The residue was diluted with water (10 mL) and extracted with ethyl acetate (2×10 mL). The aqueous layer was acidified to pH=5 with HCl (2 M), and the product was collected by filtration to give 1-[4-[[5-amino-1-[3-(trifluoromethyl)-1H-pyrazol-4-yl]imidazole-4-carbonyl]amino]-2-fluorobenzoyl]piperidine-4-carboxylic acid (370.0 mg, 0.73 mmol, 76.0% yield). MS [M+H]⁺: 510.1

### Step 7: tert-butyl 4-[1-[4-[[5-amino-1-[3-(trifluoromethyl)-1H-pyrazol-4-yl]imidazole-4-carbonyl]amino]-2-fluorobenzoyl]piperidine-4-carbonyl]piperazine-1-carboxylate

To a solution of 1-[4-[[5-amino-1-[3-(trifluoromethyl)-1H-pyrazol-4-yl]imidazole-4-carbonyl]amino]-2-fluorobenzoyl]piperidine-4-carboxylic acid (370.0 mg, 0.73 mmol, 1.0 eq) in methylene chloride (2 mL) were added 1-BOC-piperazine (162.33 mg, 0.87 mmol, 1.2 eq), N-butylphosphonic anhydride (1046.65 mg, 1.45 mmol, 2.0 eq) and N,N-diisopropylethylamine (281.61 mg, 2.18 mmol, 3.0 eq). The resulting mixture was stirred at 25° C. for 1 h. The solvent was removed under reduced pressure, and the residue was diluted with EtOAc (10 mL), washed with H₂O (3×10 mL), and dried over Na₂SO₄. Concentration under reduced pressure yielded tert-butyl 4-[1-[4-[[5-amino-1-[3-(trifluoromethyl)-1H-pyrazol-4-yl]imidazole-4-carbonyl]amino]-2-fluorobenzoyl]piperidine-4-carbonyl]piperazine-1-carboxylate (360.0 mg, 0.53 mmol, 73.1% yield). MS [M+H]⁺: 678.2

### Step 8: tert-butyl 4-[1-[4-[[5-amino-1-[1-(2-chloroallyl)-3-(trifluoromethyl)pyrazol-4-yl]imidazole-4-carbonyl]amino]-2-fluorobenzoyl]piperidine-4-carbonyl]piperazine-1-carboxylate

A mixture of tert-butyl 4-[1-[4-[[5-amino-1-[3-(trifluoromethyl)-1H-pyrazol-4-yl]imidazole-4-carbonyl]amino]-2-fluorobenzoyl]piperidine-4-carbonyl]piperazine-1-carboxylate (100.0 mg, 0.15 mmol, 1.0 eq), 2-chloroallyl 4-methylbenzenesulfonate (54.61 mg, 0.22 mmol, 1.5 eq) and potassium carbonate (61.18 mg, 0.44 mmol, 3.0 eq) in DMF (3 mL) was stirred at 25° C. for 3 h. The resulting solution was diluted with 5 ml of water and then extracted with 3×10 ml of ethyl acetate. The organic layers were combined and concentrated to dryness to afford crude product. The crude was then purified by flash column chromatography eluting with 10:1 methylene chloride:MeOH to give tert-butyl 4-[1-[4-[[5-amino-1-[1-(2-chloroallyl)-3-(trifluoromethyl)pyrazol-4-yl]imidazole-4-carbonyl]amino]-2-fluorobenzoyl]piperidine-4-carbonyl]piperazine-1-carboxylate (90.0 mg, 0.12 mmol, 81.1% yield). MS [M+H]⁺: 752.2

### Step 9: 5-amino-1-[1-(2-chloroallyl)-3-(trifluoromethyl)pyrazol-4-yl]-N-[3-fluoro-4-[4-(piperazine-1-carbonyl)piperidine-1-carbonyl]phenyl]imidazole-4-carboxamide; 1:1 2,2,2-trifluoroacetic acid; 1:1 trifluoroacetic acid

A solution of tert-butyl 4-[1-[4-[[5-amino-1-[1-(2-chloroallyl)-3-(trifluoromethyl)pyrazol-4-yl]imidazole-4-carbonyl]amino]-2-fluorobenzoyl]piperidine-4-carbonyl]piperazine-1-carboxylate (90.0 mg, 0.12 mmol, 1.0 eq) and trifluoroacetic acid (1.42 mL, 18.39 mmol, 153.7 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 20-40) to afford 5-amino-1-[1-(2-chloroallyl)-3-(trifluoromethyl)pyrazol-4-yl]-N-[3-fluoro-4-[4-(piperazine-1-carbonyl)piperidine-1-carbonyl]phenyl]imidazole-4-carboxamide;2,2,2-trifluoroacetic acid (61.7 mg, 0.08 mmol, 67.2% yield). MS [M+H]⁺: 652.1

### Compound 197

### 5-amino-1-[1-(2-chloroallyl)-3-(trifluoromethyl)pyrazol-4-yl]-N-[3-chloro-4-[4-(piperazine-1-carbonyl)piperazine-1-carbonyl]phenyl]imidazole-4-carboxamide; 1:1 2,2,2-trifluoroacetic acid

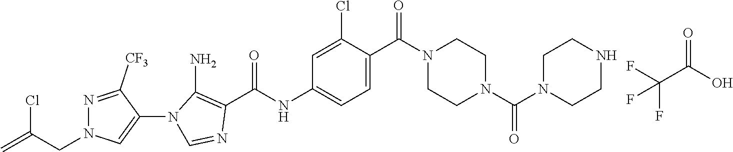

### Step 1: 4-[4-[4-[[5-amino-1-[1-[(4-methoxyphenyl)methyl]-3-(trifluoromethyl)pyrazol-4-yl]imidazole-4-carbonyl]amino]-2-chlorobenzoyl]piperazine-1-carbonyl]piperazine-1-carboxylate

To a solution of tert-butyl 4-[4-(4-amino-2-chlorobenzoyl)piperazine-1-carbonyl]piperazine-1-carboxylate (113.06 mg, 0.25 mmol, 1.0 eq) and pyridine (395.74 mg, 5.0 mmol, 20.0 eq) in methylene chloride (3 mL) was added 5-amino-1-[1-[(4-methoxyphenyl)methyl]-3-(trifluoromethyl)pyrazol-4-yl]imidazole-4-carbonyl chloride (100.0 mg, 0.25 mmol, 1.0 eq), and the mixture was stirred at 25° C. for 16 h under N₂. The resulting solution was evaporated under reduced pressure. The residue was purified by column chromatography (silica gel, 0 to 10% MeOH in methylene chloride) to afford tert-butyl 4-[4-[4-[[5-amino-1-[1-[(4-methoxyphenyl)methyl]-3-(trifluoromethyl)pyrazol-4-yl]imidazole-4-carbonyl]amino]-2-chlorobenzoyl]piperazine-1-carbonyl]piperazine-1-carboxylate (127.0 mg, 0.16 mmol, 56.7% yield). MS [M+H]⁺: 815.3

### Step 2: 5-amino-N-[3-chloro-4-[4-(piperazine-1-carbonyl)piperazine-1-carbonyl]phenyl]-1-[3-(trifluoromethyl)-1H-pyrazol-4-yl]imidazole-4-carboxamide

A solution of tert-butyl 4-[4-[4-[[5-amino-1-[1-[(4-methoxyphenyl)methyl]-3-(trifluoromethyl)pyrazol-4-yl]imidazole-4-carbonyl]amino]-2-chlorobenzoyl]piperazine-1-carbonyl]piperazine-1-carboxylate (1500.0 mg, 1.84 mmol, 1.0 eq) in trifluoracetic acid (15.0 mL, 1.84 mmol, 1.0 eq) was stirred at 70° C. for 16 h. The reaction was concentrated to dryness to afford crude product 5-amino-N-[3-chloro-4-[4-(piperazine-1-carbonyl)piperazine-1-carbonyl]phenyl]-1-[3-(trifluoromethyl)-1H-pyrazol-4-yl]imidazole-4-carboxamide (650.0 mg, 1.09 mmol, 33.8% yield). MS [M+H]⁺: 595.0

### Step 3: tert-butyl 4-[4-[4-[[5-amino-1-[3-(trifluoromethyl)-1H-pyrazol-4-yl]imidazole-4-carbonyl]amino]-2-chlorobenzoyl]piperazine-1-carbonyl]piperazine-1-carboxylate

To a solution of 5-amino-N-[3-chloro-4-[4-(piperazine-1-carbonyl)piperazine-1-carbonyl]phenyl]-1-[3-(trifluoromethyl)-1H-pyrazol-4-yl]imidazole-4-carboxamide (650.0 mg, 1.09 mmol, 1.0 eq) in THF (5 mL) and water (0.500 mL) was added di-tert-butyl dicarbonate (119.22 mg, 0.55 mmol, 0.5 eq) and sodium bicarbonate (275.34 mg, 3.28 mmol, 3.0 eq). The reaction was stirred at 25° C. for 6 h. The reaction was concentrated to dryness and the residue was taken up in EtOAc (20 mL) and the organics washed with 2×20 ml water and 20 mL brine. The organics were then separated and dried (MgSO₄) before concentrating to dryness. The crude was then purified by flash column chromatography eluting with 100% EtOAc to afford tert-butyl 4-[4-[4-[[5-amino-1-[3-(trifluoromethyl)-1H-pyrazol-4-yl]imidazole-4-carbonyl]amino]-2-chlorobenzoyl]piperazine-1-carbonyl]piperazine-1-carboxylate (450.0 mg, 0.65 mmol, 48.8% yield). MS [M+H]⁺: 695.9

### Step 4: tert-butyl 4-[4-[4-[[5-amino-1-[1-(2-chloroallyl)-3-(trifluoromethyl)pyrazol-4-yl]imidazole-4-carbonyl]amino]-2-chlorobenzoyl]piperazine-1-carbonyl]piperazine-1-carboxylate

A solution of tert-butyl 4-[4-[4-[[5-amino-1-[3-(trifluoromethyl)-1H-pyrazol-4-yl]imidazole-4-carbonyl]amino]-2-chlorobenzoyl]piperazine-1-carbonyl]piperazine-1-carboxylate (100.0 mg, 0.14 mmol, 1.0 eq) in DMF (3 mL) was added potassium carbonate (39.77 mg, 0.29 mmol, 2.0 eq) and 2-chloroallyl 4-methylbenzenesulfonate (106.48 mg, 0.43 mmol, 3.0 eq). The reaction mixture was stirred at 25° C. for 6 h. The reaction was concentrated to dryness and the residue was taken up in EtOAc (20 mL) and the organics washed with 2×10 mL water followed by 10 mL brine. The organics were then separated and dried (MgSO₄) before concentrating to dryness. The crude was then purified by flash column chromatography eluting with 10% MeOH in methylene chloride to afford tert-butyl 4-[4-[4-[[5-amino-1-[1-(2-chloroallyl)-3-(trifluoromethyl)pyrazol-4-yl]imidazole-4-carbonyl]amino]-2-chlorobenzoyl]piperazine-1-carbonyl]piperazine-1-carboxylate (78.0 mg, 0.1 mmol, 60.4% yield). MS [M+H]⁺: 768.9

### Step 5: 5-amino-1-[1-(2-chloroallyl)-3-(trifluoromethyl)pyrazol-4-yl]-N-[3-chloro-4-[4-(piperazine-1-carbonyl)piperazine-1-carbonyl]phenyl]imidazole-4-carboxamide; 1:1 2,2,2-trifluoroacetic acid

To a solution of tert-butyl 4-[4-[4-[[5-amino-1-[1-(2-chloroallyl)-3-(trifluoromethyl)pyrazol-4-yl]imidazole-4-carbonyl]amino]-2-chlorobenzoyl]piperazine-1-carbonyl]piperazine-1-carboxylate (78.0 mg, 0.1 mmol, 1.0 eq) in methylene chloride (3 mL) was added trifluoroacetic acid (1.0 mL). The reaction was stirred at 25° C. for 2 h. The reaction was concentrated to dryness to afford a crude product. The crude was purified by Prep-HPLC (Gemini-C18 150×21.2 mm, 5 μm, MeCN-H₂O (0.1% TFA), 15-40) to afford 5-amino-1-[1-(2-chloroallyl)-3-(trifluoromethyl)pyrazol-4-yl]-N-[3-chloro-4-[4-(piperazine-1-carbonyl)piperazine-1-carbonyl]phenyl]imidazole-4-carboxamide; 1:1 2,2,2-trifluoroacetic acid (55.8 mg, 0.07 mmol, 70% yield). MS [M+H]⁺: 668.9

### Compound 198

### 5-amino-N-[4-[4-[(2R)-azetidine-2-carbonyl]piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid

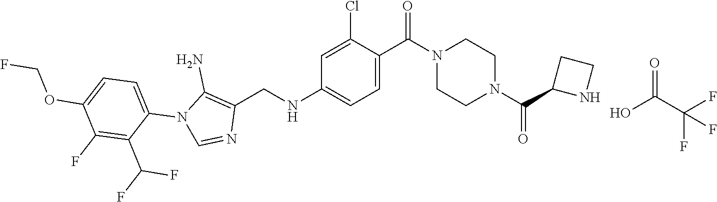

### Step 1: tert-butyl(2R)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidine-1-carboxylate

To a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide (70.0 mg, 0.13 mmol, 1.0 eq) in methylene chloride (2 mL) were added (R)-1-(tert-butoxycarbonyl) azetidine-2-carboxylic acid (28.64 mg, 0.14 mmol, 1.1 eq),N,N-diisopropylethylamine (50.18 mg, 0.39 mmol, 3.0 eq) and 1,3,5,2,4,6-trioxatriphosphorinane, 2,4,6-tributyl-, 2,4,6-trioxide (186.49 mg, 0.26 mmol, 2.0 eq) under N₂ atmosphere. The resulting mixture was stirred at 25° C. for 1 h. The reaction mixture was then diluted with EtOAc (30 mL), washed with H₂O (3×15 mL), dried over Na₂SO₄ and concentrated under reduced pressure. The residue was purified by flash column chromatography (95:5 methylene chloride:MeOH) to give tert-butyl(2R)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidine-1-carboxylate (78.0 mg, 0.11 mmol, 79.9% yield). MS [M−Boc+H]⁺: 623.9

### Step 2: 5-amino-N-[4-[4-[(2R)-azetidine-2-carbonyl]piperazine-1-carbonyl]-3-chlorophenyl]-1-[2-(difluoromethyl)-3-fluoro-4-(fluoromethoxy)phenyl]imidazole-4-carboxamide; 1:1 2,2,2-trifluoroacetic acid

To a solution of tert-butyl(2R)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]azetidine-1-carboxylate (78.0 mg, 0.11 mmol, 1.0 eq) in methylene chloride (1.0 mL) was added trifluoroacetic acid (0.5 mL, 6.53 mmol, 472.81 eq). The resulting mixture was stirred at 25° C. for 1 h. After concentration under reduced pressure, the residue was purified by prep-HPLC [Gemini-C18 150×21.2 mm; 5 μm; MeCN/H₂O (0.1% TFA), 35%-50%] to give 5-amino-N-[4-[4-[(2R)-azetidine-2-carbonyl]piperazine-1-carbonyl]-3-chloro-phenyl]-1-[2-(difluoromethyl)-3-fluoro-4-(fluoromethoxy)phenyl]imidazole-4-carboxamide; 1:1 2,2,2-trifluoroacetic acid (63.1 mg, 0.09 mmol, 79.4% yield). MS [M+H]⁺: 623.9

### Compound 199

### N-[4-[4-[(2S,3aR,6aS)-2,3,3a,4,6,6a-hexahydro-1H-furo[3,4-b]pyrrole-2-carbonyl]piperazine-1-carbonyl]-3-chlorophenyl]-5-amino-1-[2,3-difluoro-4-(fluoromethoxy)phenyl]imidazole-4-carboxamide

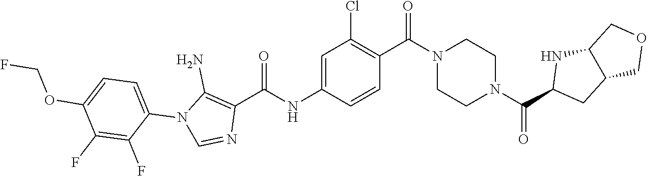 [SMILES: Nc1c(C(=O)Nc2ccc(C(=O)N3CCN(C(=O)[C@@H]4C[C@H]5COC[C@H]5N4)CC3)c(Cl)c2)ncn1-c1ccc(OCF)c(F)c1F]

### Step 1: 12-phenyl-4,10-dioxa-1-azatricyclo[6.4.002,6]dodecan-9-one

To a solution of N,N-diisopropylethylamine (2.95 mL, 16.93 mmol, 1.0 eq) and (5S)-5-phenylmorpholin-2-one (3000.0 mg, 16.93 mmol, 1.0 eq) in toluene (30 mL) was added a suspension of 2-allyloxyacetaldehyde (5085.1 mg, 50.79 mmol, 3.0 eq) in toluene (30 mL) at 140° C. for 16 h under N₂. The reaction was concentrated to dryness. The residue was taken up in EtOAc (100 ml) and the organics washed with 2×10 ml water followed by 10 mL brine. The organics were then separated and dried (Na₂SO₄) before concentrating to dryness. The crude was then purified by flash column chromatography eluting with 20% EtOAc in petroleum ether to (2S,6R,8S,12S)-12-phenyl-4,10-dioxa-1-azatricyclo[6.4.002,6]dodecan-9-one (1800.0 mg, 6.94 mmol, 41.0% yield). MS [M+H]⁺: 260.0

### Step 2: methyl(2S,3aR,6aS)-1-[(1S)-2-hydroxy-1-phenylethyl]-2,3,3a,4,6,6a-hexahydrofuro[3,4-b]pyrrole-2-carboxylate

A solution of (2S,6R,8S,12S)-12-phenyl-4,10-dioxa-1-azatricyclo[6.4.002,6]dodecan-9-one (1000.0 mg, 3.86 mmol, 1.0 eq), trifluoroacetic acid (1.86 mL, 25.07 mmol, 6.5 eq) and palladium hydroxide (541.59 mg, 0.77 mmol, 0.2 eq) in methanol (15 mL) was stirred at 25° C. for 16 h under hydrogen (777.36 mg, 385.65 mmol, 100.0 eq). The solid was filtered out. The crude was then purified by flash column chromatography eluting with 40% EtOAc in petroleum ether to give the methyl(2S,3aR,6aS)-1-[(1S)-2-hydroxy-1-phenylethyl]-2,3,3a,4,6,6a-hexahydrofuro[3,4-b]pyrrole-2-carboxylate (900.0 mg, 3.09 mmol, 80.1% yield). MS [M+H]⁺: 292.1

### Step 3: methyl(2S,3aR,6aS)-2,3,3a,4,6,6a-hexahydro-1H-furo[3,4-b]pyrrole-2-carboxylate

A solution of methyl(2S,3aR,6aS)-1-[(1S)-2-hydroxy-1-phenylethyl]-2,3,3a,4,6,6a-hexahydrofuro[3,4-b]pyrrole-2-carboxylate (900.0 mg, 3.09 mmol, 1.0 eq), hydrochloric acid (0.44 mL, 5.31 mmol, 1.72 eq) and palladium hydroxide (86.76 mg, 0.62 mmol, 0.2 eq) in methanol (30 mL) was stirred at 70° C. for 5 h under hydrogen (1000.0 mL, 3.09 mmol, 1.0 eq). The solid was filtered out. The crude was then purified by flash column chromatography eluting with 40% EtOAc in petroleum ether to give the methyl(2S,3aR,6aS)-2,3,3a,4,6,6a-hexahydro-1H-furo[3,4-b]pyrrole-2-carboxylate (450.0 mg, 2.63 mmol, 85.1% yield). MS [M+H]⁺: 172.1

### Step 4: (2S,3aR,6aS)-2,3,3a,4,6,6a-hexahydro-1H-furo[3,4-b]pyrrole-2-carboxylic acid

Lithium hydroxide (34.97 mg, 1.46 mmol, 5.0 eq) was added to a solution of methyl(2S,3aR,6aS)-2,3,3a,4,6,6a-hexahydro-1H-furo[3,4-b]pyrrole-2-carboxylate (50.0 mg, 0.29 mmol, 1.0 eq) in THF (2 mL) and water (2 mL). The resulting mixture was stirred at 50° C. for 2 h. The reaction was concentrated to dryness and the residue was taken up in EtOAc (2 mL) and the organics washed with 2×3 mL water followed by 3 mL brine. The water fractions were concentrated to dryness to give (2S,3aR,6aS)-2,3,3a,4,6,6a-hexahydro-1H-furo[3,4-b]pyrrole-2-carboxylic acid (25.0 mg, 0.16 mmol, 54.5% yield). MS [M+H]⁺: 158.1

### Step 5: N-[4-[4-[(2S,3aR,6aS)-2,3,3a,4,6,6a-hexahydro-1H-furo[3,4-b]pyrrole-2-carbonyl]piperazine-1-carbonyl]-3-chlorophenyl]-5-amino-1-[2,3-difluoro-4-(fluoromethoxy)phenyl]imidazole-4-carboxamide

To a solution of (2S,3aR,6aS)-2,3,3a,4,6,6a-hexahydro-1H-furo[3,4-b]pyrrole-2-carboxylic acid (41.96 mg, 0.27 mmol, 1.0 eq) and 5-amino-N-[3-chloro-4-(piperazine-1-carbonyl)phenyl]-1-[2,3-difluoro-4-(fluoromethoxy)phenyl]imidazole-4-carboxamide (135.87 mg, 0.27 mmol, 1.0 eq) in methylene chloride (1 mL) was added 2-(7-azabenzotriazol-1-yl)-N,N,N′,N′-tetramethyluronium hexafluorophosphate (203.04 mg, 0.53 mmol, 2.0 eq) and triethylamine (0.11 mL, 0.8 mmol, 3.0 eq). The reaction mixture was stirred at 25° C. for 2 h. The reaction was then quenched with water (25 mL). The resulting solution was extracted with ethyl acetate (3×20 mL). The combined organic phases were dried over anhydrous sodium sulfate and concentrated under reduced pressure. The residue was purified by flash column to give N-[4-[4-[(2S,3aR,6aS)-2,3,3a,4,6,6a-hexahydro-1H-furo[3,4-b]pyrrole-2-carbonyl]piperazine-1-carbonyl]-3-chlorophenyl]-5-amino-1-[2,3-difluoro-4-(fluoromethoxy)phenyl]imidazole-4-carboxamide (8.4 mg, 0.01 mmol, 4.8% yield). MS [M+H]⁺: 648.0

### Compound 200

### 5-amino-1-[1-(cyclopropylmethyl)-3-(trifluoromethyl)pyrazol-4-yl]-N-[3-fluoro-4-[4-(4-fluoropiperidine-4-carbonyl)piperazine-1-carbonyl]phenyl]imidazole-4-carboxamide;2,2,2-trifluoroacetic acid

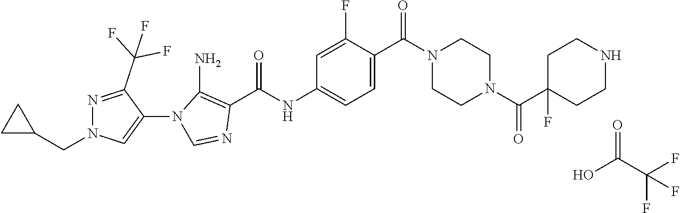

### Step 1: tert-butyl 4-[4-[4-[[5-amino-1-[3-(trifluoromethyl)-1H-pyrazol-4-yl]imidazole-4-carbonyl]amino]-2-fluorobenzoyl]piperazine-1-carbonyl]-4-fluoropiperidine-1-carboxylate

To a solution of 5-amino-N-[3-fluoro-4-(piperazine-1-carbonyl)phenyl]-1-[3-(trifluoromethyl)-1H-pyrazol-4-yl]imidazole-4-carboxamide (200.0 mg, 0.43 mmol, 1.0 eq) in methylene chloride (2 mL) were added 1-BOC-4-fluoro-4-piperidinecarboxylic acid (106.03 mg, 0.43 mmol, 1.0 eq), 1,3,5,2,4,6-Trioxatriphosphorinane (617.94 mg, 0.86 mmol, 2.0 eq) and N,N-diisopropylethylamine (166.26 mg, 1.29 mmol, 3.0 eq). The resulting mixture was stirred at 25° C. for 1 h. The reaction was concentrated to dryness to afford crude product. The crude was then purified by flash column chromatography eluting with 10:1 methylene chloride:MeOH to give tert-butyl 4-[4-[4-[[5-amino-1-[3-(trifluoromethyl)-1H-pyrazol-4-yl]imidazole-4-carbonyl]amino]-2-fluorobenzoyl]piperazine-1-carbonyl]-4-fluoropiperidine-1-carboxylate (238.0 mg, 0.34 mmol, 79.8% yield). MS [(M−Boc)+H]⁺: 696.3

### Step 2: tert-butyl 4-[4-[4-[[5-amino-1-[1-(cyclopropylmethyl)-3-(trifluoromethyl)pyrazol-4-yl]imidazole-4-carbonyl]amino]-2-fluorobenzoyl]piperazine-1-carbonyl]-4-fluoropiperidine-1-carboxylate

A solution of tert-butyl 4-[4-[4-[[5-amino-1-[3-(trifluoromethyl)-1H-pyrazol-4-yl]imidazole-4-carbonyl]amino]-2-fluorobenzoyl]piperazine-1-carbonyl]-4-fluoropiperidine-1-carboxylate (60.0 mg, 0.09 mmol, 1.0 eq), (bromomethyl)cyclopropane (0.01 mL, 0.13 mmol, 1.5 eq) and potassium carbonate (35.76 mg, 0.26 mmol, 3.0 eq) in DMF (1 mL) was stirred at 25° C. for 1 h, then concentrated under reduced pressure at 30° C. The resulting residue was purified by flash chromatography to tert-butyl 4-[4-[4-[[5-amino-1-[1-(cyclopropylmethyl)-3-(trifluoromethyl)pyrazol-4-yl]imidazole-4-carbonyl]amino]-2-fluorobenzoyl]piperazine-1-carbonyl]-4-fluoropiperidine-1-carboxylate (45.0 mg, 0.06 mmol, 69.6% yield). MS [(M−Boc)+H]⁺: 650.0

### Step 3: 5-amino-1-[1-(cyclopropylmethyl)-3-(trifluoromethyl)pyrazol-4-yl]-N-[3-fluoro-4-[4-(4-fluoropiperidine-4-carbonyl)piperazine-1-carbonyl]phenyl]imidazole-4-carboxamide;2,2,2-trifluoroacetic acid

A solution of tert-butyl 4-[4-[4-[[5-amino-1-[1-(cyclopropylmethyl)-3-(trifluoromethyl)pyrazol-4-yl]imidazole-4-carbonyl]amino]-2-fluorobenzoyl]piperazine-1-carbonyl]-4-fluoropiperidine-1-carboxylate (35.0 mg, 0.05 mmol, 1.0 eq) and trifluoroacetic acid (5.32 mg, 0.05 mmol, 1.0 eq) in methylene chloride (1 mL) was stirred at 25° C. for 1 h. The resulting solution was concentrated under reduced pressure. The crude product was purified by Prep-HPLC (Gemini-C18 150×21.2 mm, 5 μm, MeCN-H₂O (0.1% TFA), 15%-45%) to give 5-amino-1-[1-(cyclopropylmethyl)-3-(trifluoromethyl)pyrazol-4-yl]-N-[3-fluoro-4-[4-(4-fluoropiperidine-4-carbonyl)piperazine-1-carbonyl]phenyl]imidazole-4-carboxamide;2,2,2-trifluoroacetic acid (9.3 mg, 0.01 mmol, 24% yield). MS [M+H]⁺: 650.3

### Compound 201

### 5-amino-1-[1-(2-chloroallyl)-3-(trifluoromethyl)pyrazol-4-yl]-N-[3-fluoro-4-[4-(4-fluoropiperidine-4-carbonyl)piperazine-1-carbonyl]phenyl]imidazole-4-carboxamide; 1:1 2,2,2-trifluoroacetic acid

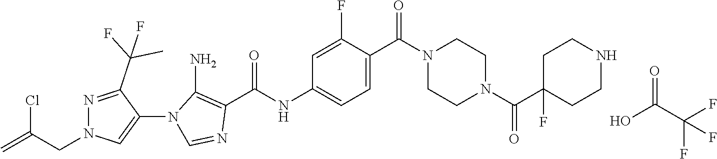

### Step 1: tert-butyl 4-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]-4-fluoropiperidine-1-carboxylate

A solution of tert-butyl 4-[4-[4-[[5-amino-1-[3-(trifluoromethyl)-1H-pyrazol-4-yl]imidazole-4-carbonyl]amino]-2-fluorobenzoyl]piperazine-1-carbonyl]-4-fluoropiperidine-1-carboxylate (82.0 mg, 0.12 mmol, 1.0 eq), 2-chloroallyl 4-methylbenzenesulfonate (43.62 mg, 0.18 mmol, 1.5 eq) and potassium carbonate (48.87 mg, 0.35 mmol, 3.0 eq) in DMF (3 mL) was stirred at 25° C. for 2 h. The resulting solution was diluted with 5 ml of water, then extracted with 3×5 mL EtOAc. The organic layers were combined, dried and concentrated under reduced pressure. The residue was applied on a silica gel column and eluted with 10:1 methylene chloride:MeOH to give tert-butyl 4-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]-4-fluoropiperidine-1-carboxylate (67.0 mg, 0.09 mmol, 73.8% yield). MS [M−Boc+H]⁺: 670.1

### Step 2: 5-amino-1-[1-(2-chloroallyl)-3-(trifluoromethyl)pyrazol-4-yl]-N-[3-fluoro-4-[4-(4-fluoropiperidine-4-carbonyl)piperazine-1-carbonyl]phenyl]imidazole-4-carboxamide; 1:1 2,2,2-trifluoroacetic acid

A solution of tert-butyl 4-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]-4-fluoropiperidine-1-carboxylate (67.0 mg, 0.09 mmol, 1.0 eq), trifluoroacetic acid (1.0 mL, 12.98 mmol, 149.2 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 30-60) to afford 5-amino-1-[1-(2-chloroallyl)-3-(trifluoromethyl)pyrazol-4-yl]-N-[3-fluoro-4-[4-(4-fluoropiperidine-4-carbonyl)piperazine-1-carbonyl]phenyl]imidazole-4-carboxamide;1:1 2,2,2-trifluoroacetic acid (44.2 mg, 0.06 mmol, 64.8% yield). MS [M+H]⁺: 670.1

### Compound 202

### 5-amino-1-[1-(2-fluoroallyl)-3-(trifluoromethyl)pyrazol-4-yl]-N-[3-fluoro-4-[4-(4-fluoropiperidine-4-carbonyl)piperazine-1-carbonyl]phenyl]imidazole-4-carboxamide; 1:1 2,2,2-trifluoroacetic acid

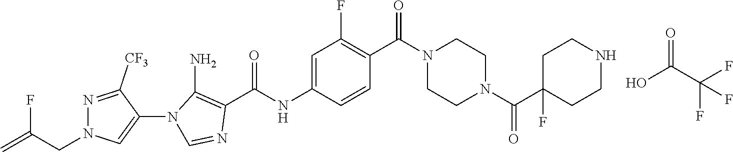

### Step 1: tert-butyl 4-fluoro-4-[4-(2-fluoro-4-nitrobenzoyl)piperazine-1-carbonyl]piperidine-1-carboxylate

To a solution of (2-fluoro-4-nitrophenyl)-piperazin-1-yl-methanone hydrochloride (200.0 mg, 0.69 mmol, 1.0 eq) in methylene chloride (5 mL) were added 1-BOC-4-fluoro-4-piperidine-carboxylic acid (179.25 mg, 0.72 mmol, 1.05 eq), 1,3,5,2,4,6-trioxatriphosphorinane, 2,4,6-tributyl-, 2,4,6-trioxide (596.93 mg, 0.83 mmol, 1.2 eq) and N,N-diisopropylethylamine (178.45 mg, 1.38 mmol, 2.0 eq). The resulting mixture was stirred at 25° C. for 1 h. The solvent was removed under reduced pressure, and the residue was diluted with EtOAc (30 mL), washed with H₂O (4×15 mL), dried over Na₂SO₄ and concentrated under reduced pressure. The residue was purified by flash column chromatography (1:1 petroleum ether:EtOAc) to give tert-butyl 4-fluoro-4-[4-(2-fluoro-4-nitrobenzoyl)piperazine-1-carbonyl]piperidine-1-carboxylate (230.0 mg, 0.48 mmol, 69.0% yield). MS [M+Na]⁺: 505.0

### Step 2: tert-butyl 4-[4-(4-amino-2-fluorobenzoyl)piperazine-1-carbonyl]-4-fluoropiperidine-1-carboxylate

To a solution of tert-butyl 4-fluoro-4-[4-(2-fluoro-4-nitrobenzoyl)piperazine-1-carbonyl]piperidine-1-carboxylate (230.0 mg, 0.48 mmol, 1.0 eq) in methanol (20 mL) was added palladium (carbon-supported, spherical particles)(50.0 mg) under hydrogen (0.96 mg, 0.48 mmol, 1.0 eq) atmosphere. The resulting mixture was stirred at 25° C. for 15 h. The Pd/C was filtered off and the solvent was removed under reduced pressure to give tert-butyl 4-[4-(4-amino-2-fluorobenzoyl)piperazine-1-carbonyl]-4-fluoropiperidine-1-carboxylate (146.0 mg, 0.32 mmol, 65.6% yield). MS [M+Na]⁺: 475.0

### Step 3: tert-butyl 4-[4-[4-[[5-amino-1-[1-(2-fluoroallyl)-3-(trifluoromethyl)pyrazol-4-yl]imidazole-4-carbonyl]amino]-2-fluorobenzoyl]piperazine-1-carbonyl]-4-fluoropiperidine-1-carboxylate

To a solution of tert-butyl 4-[4-(4-amino-2-fluorobenzoyl)piperazine-1-carbonyl]-4-fluoropiperidine-1-carboxylate (144.73 mg, 0.32 mmol, 0.6 eq) in methylene chloride (3 mL) were added pyridine (0.43 mL, 5.33 mmol, 10.0 eq) and 5-amino-1-[1-(2-fluoroallyl)-3-(trifluoromethyl)pyrazol-4-yl]imidazole-4-carbonyl chloride (180.0 mg, 0.53 mmol, 1.0 eq) in order under N₂ atmosphere. The resulting mixture was stirred at 25° C. for 1 h. The reaction mixture was diluted with methylene chloride (30 mL), washed with citric acid aqueous solution (15 mL×3), dried over Na₂SO₄, and concentrated under reduced pressure. The residue was purified by flash column chromatography (95:5 methylene chloride:MeOH) to give tert-butyl 4-[4-[4-[[5-amino-1-[1-(2-fluoroallyl)-3-(trifluoromethyl)pyrazol-4-yl]imidazole-4-carbonyl]amino]-2-fluorobenzoyl]piperazine-1-carbonyl]-4-fluoropiperidine-1-carboxylate (330.0 mg, 0.44 mmol, 34.5% yield). MS [M−Boc+H]⁺: 654.0

### Step 4: 5-amino-1-[1-(2-fluoroallyl)-3-(trifluoromethyl)pyrazol-4-yl]-N-[3-fluoro-4-[4-(4-fluoropiperidine-4-carbonyl)piperazine-1-carbonyl]phenyl]imidazole-4-carboxamide; 1:1 2,2,2-trifluoroacetic acid

To a solution of tert-butyl 4-[4-[4-[[5-amino-1-[1-(2-fluoroallyl)-3-(trifluoromethyl)pyrazol-4-yl]imidazole-4-carbonyl]amino]-2-fluorobenzoyl]piperazine-1-carbonyl]-4-fluoropiperidine-1-carboxylate (330.0 mg, 0.18 mmol, 1.0 eq) in methylene chloride (3 mL) was added trifluoroacetic acid (2.0 mL, 26.12 mmol, 142.03 eq) under N₂ atmosphere. The resulting mixture was stirred at 25° C. for 1 h. After concentration under reduced pressure, the residue was purified by prep-HPLC [Gemini-C18 150×21.2 mm, 5 μm, MeCN— H₂O (0.1% TFA), 25%˜35%] to give 5-amino-1-[1-(2-fluoroallyl)-3-(trifluoromethyl)pyrazol-4-yl]-N-[3-fluoro-4-[4-(4-fluoropiperidine-4-carbonyl)piperazine-1-carbonyl]phenyl]imidazole-4-carboxamide;2,2,2-trifluoroacetic acid (58.2 mg, 0.08 mmol, 39.6% yield). MS [M+H]⁺: 654.0

### Compound 203

### 5-amino-N-[3-chloro-4-[4-(4-fluoropiperidine-4-carbonyl)piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid

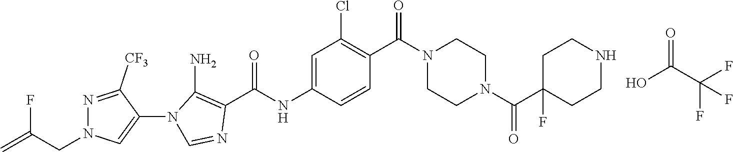

### Step 1: tert-butyl 4-[4-[4-[[5-amino-1-[3-(trifluoromethyl)-1H-pyrazol-4-yl]imidazole-4-carbonyl]amino]-2-chlorobenzoyl]piperazine-1-carbonyl]-4-fluoropiperidine-1-carboxylate

A solution of 5-amino-N-[3-chloro-4-(piperazine-1-carbonyl)phenyl]-1-[3-(trifluoromethyl)-1H-pyrazol-4-yl]imidazole-4-carboxamide (200.0 mg, 0.41 mmol, 1.0 eq), 1-BOC-4-fluoro-4-piperidinecarboxylic acid (102.42 mg, 0.41 mmol, 1.0 eq), propylphosphonic anhydride (1317.92 mg, 2.07 mmol, 5.0 eq) and N,N-diisopropylethylamine (0.43 mL, 2.49 mmol, 6.0 eq) was stirred at 25° C. for 3 h. The reaction was concentrated to dryness and the residue was taken up in EtOAc (50 mL). The organics were washed with 2×10 mL water followed by 10 mL brine. The organics were then separated and dried (Na₂SO₄) before concentrating to dryness. The crude was then purified by flash column chromatography eluting with 60% EtOAc in petroleum ether to give tert-butyl 4-[4-[4-[[5-amino-1-[3-(trifluoromethyl)-1H-pyrazol-4-yl]imidazole-4-carbonyl]amino]-2-chlorobenzoyl]piperazine-1-carbonyl]-4-fluoropiperidine-1-carboxylate (180.0 mg, 0.25 mmol, 61.0% yield). MS [(M−Boc)+H]⁺: 612.2.

### Step 2: tert-butyl 4-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-4-fluoropiperidine-1-carboxylate

To a solution of tert-butyl 4-[4-[4-[[5-amino-1-[3-(trifluoromethyl)-1H-pyrazol-4-yl]imidazole-4-carbonyl]amino]-2-chlorobenzoyl]piperazine-1-carbonyl]-4-fluoropiperidine-1-carboxylate (90.0 mg, 0.13 mmol, 1.0 eq) in DMF (1 mL) were added 2-fluoroallyl 4-methylbenzenesulfonate (34.92 mg, 0.15 mmol, 1.2 eq) and potassium carbonate (52.4 mg, 0.38 mmol, 3.0 eq). The reaction was stirred at 25° C. for 3 h. The reaction was concentrated to dryness, and the residue was taken up in EtOAc (20 ml) and washed with 2×10 ml water followed 10 ml brine. The organics were then separated and dried (Na₂SO₄) before concentration to dryness to give tert-butyl 4-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-4-fluoropiperidine-1-carboxylate (70.0 mg, 0.09 mmol, 71.9% yield). MS [(M−Boc)+H]⁺: 669.9

### Step 3: 5-amino-N-[3-chloro-4-[4-(4-fluoropiperidine-4-carbonyl)piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid

A solution of tert-butyl 4-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-4-fluoropiperidine-1-carboxylate (60.0 mg, 0.08 mmol, 1.0 eq) and trifluoroacetic acid (1.0 mL, 12.98 mmol, 166.61 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA), 25%-40%) to give 5-amino-N-[3-chloro-4-[4-(4-fluoropiperidine-4-carbonyl)piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid (8.9 mg, 0.01 mmol, 14.5% yield). MS [M+H]⁺: 669.9

### Compound 204

### 5-amino-1-[2-(difluoromethyl)-3-fluoro-4-(fluoromethoxy)phenyl]-N-[3-fluoro-4-[4-(4-fluoropiperidine-4-carbonyl)piperazine-1-carbonyl]phenyl]imidazole-4-carboxamide; 1:1 2,2,2-trifluoroacetic acid

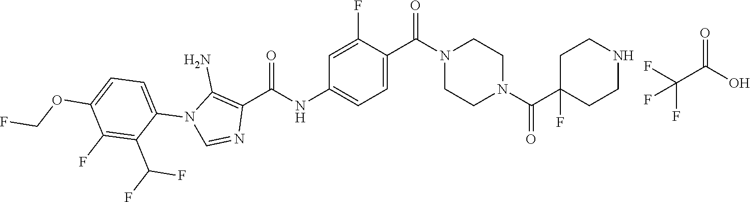

### Step 1: tert-butyl 4-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-4-fluoropiperidine-1-carboxylate

To a solution of 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (60.0 mg, 0.11 mmol, 1.0 eq) in methylene chloride (2 mL) were added 1-Boc-4-fluoro-4-piperidinecarboxylic acid (31.12 mg, 0.13 mmol, 1.1 eq), 1,3,5,2,4,6-trioxatriphosphorinane (164.86 mg, 0.23 mmol, 2.0 eq) and N,N-diisopropylethylamine (44.36 mg, 0.34 mmol, 3.0 eq). The resulting mixture was stirred at 25° C. for 1 h. The solvent was removed under reduced pressure, and the residue was diluted with EtOAc (100 mL), washed with H₂O (4×60 mL), dried over Na₂SO₄ and concentrated under reduced pressure. The residue was purified by flash column chromatography (4:1 isohexane:EtOAc) to afford tert-butyl 4-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-4-fluoropiperidine-1-carboxylate (70.0 mg, 0.09 mmol, 81.2% yield). MS [M−Boc+H]⁺: 655.3

### Step 2: 5-amino-1-[2-(difluoromethyl)-3-fluoro-4-(fluoromethoxy)phenyl]-N-[3-fluoro-4-[4-(4-fluoropiperidine-4-carbonyl)piperazine-1-carbonyl]phenyl]imidazole-4-carboxamide; 1:1 2,2,2-trifluoroacetic acid

To a solution of tert-butyl 4-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-4-fluoropiperidine-1-carboxylate (68.0 mg, 0.09 mmol, 1.0 eq) in methylene chloride (2 mL) was added trifluoroacetic acid (2.32 mL, 30.3 mmol, 335 eq). The resulting mixture was stirred at 25° C. for 1 h. The mixture was concentrated under reduced pressure. The residue was purified by prep-HPLC [Gemini-C18 150×21.2 mm, 5 μm, MeCN-H₂O (0.1% TFA)] to afford 5-amino-1-[2-(difluoromethyl)-3-fluoro-4-(fluoromethoxy)phenyl]-N-[3-fluoro-4-[4-(4-fluoropiperidine-4-carbonyl)piperazine-1-carbonyl]phenyl]imidazole-4-carboxamide; 1:1 2,2,2-trifluoroacetic acid (51.2 mg, 0.07 mmol, 71.0% yield). MS [M+H]⁺: 655.3

### Compound 205

### 5-amino-1-[2-(difluoromethyl)-3-fluoro-4-(fluoromethoxy)phenyl]-N-[3-fluoro-4-[4-[(1R,5S,6r)-6-fluoro-3-azabicyclo[3.1.0]hexane-6-carbonyl]piperazine-1-carbonyl]phenyl]imidazole-4-carboxamide; 1:1 2,2,2-trifluoroacetic acid

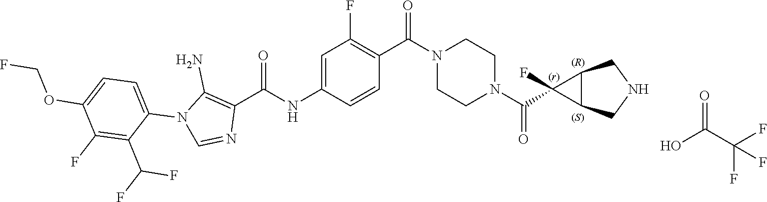

### Step 1: 5-amino-1-[2-(difluoromethyl)-3-fluoro-4-(fluoromethoxy)phenyl]imidazole-4-carbonyl chloride

5-Amino-1-[2-(difluoromethyl)-3-fluoro-4-(fluoromethoxy)phenyl]imidazole-4-carboxylic acid (100.0 mg, 0.31 mmol, 1.0 eq) was dissolved in thionyl chloride (4.0 mL, 55.14 mmol, 176.02 eq) at 25° C. under N₂, and the solution was stirred at 25° C. for 1 h under N₂. The resulting solution was evaporated to dryness and azeotroped with methylene chloride to give 5-amino-1-[2-(difluoromethyl)-3-fluoro-4-(fluoromethoxy)phenyl]imidazole-4-carbonyl chloride (100.0 mg, 0.3 mmol, 50.1% yield), which was used without further purification. MS [M−Cl+OMe+H]⁺: 334.0

### Step 2: tert-butyl 4-[4-[[5-amino-1-[2-(difluoromethyl)-3-fluoro-4-(fluoromethoxy)phenyl]imidazole-4-carbonyl]amino]-2-fluorobenzoyl]piperazine-1-carboxylate

A solution of tert-butyl 4-(4-amino-2-fluorobenzoyl)piperazine-1-carboxylate (76.61 mg, 0.24 mmol, 0.8 eq) and pyridine (0.48 mL, 5.92 mmol, 20.0 eq) in methylene chloride (2 mL) was stirred at 25° C. Next, a solution of 5-amino-1-[2-(difluoromethyl)-3-fluoro-4-(fluoromethoxy)phenyl]imidazole-4-carbonyl chloride (100.0 mg, 0.3 mmol, 1.0 eq) in methylene chloride (2 mL) was added, and the mixture was stirred at 25° C. for 1 h under N₂. The resulting solution was concentrated under reduced pressure. The residue was applied on a silica gel column and eluted with 10:1 methylene chloride:MeOH to give tert-butyl 4-[4-[[5-amino-1-[2-(difluoromethyl)-3-fluoro-4-(fluoromethoxy)phenyl]imidazole-4-carbonyl]amino]-2-fluorobenzoyl]piperazine-1-carboxylate (85.0 mg, 0.14 mmol, 31.7% yield). MS [M+H]⁺: 625.1

### Step 3: 5-amino-1-[2-(difluoromethyl)-3-fluoro-4-(fluoromethoxy)phenyl]-N-[3-fluoro-4-(piperazine-1-carbonyl)phenyl]imidazole-4-carboxamide

A solution of tert-butyl 4-[4-[[5-amino-1-[2-(difluoromethyl)-3-fluoro-4-(fluoromethoxy)phenyl]imidazole-4-carbonyl]amino]-2-fluorobenzoyl]piperazine-1-carboxylate (85.0 mg, 0.14 mmol, 1.0 eq) and trifluoroacetic acid (1.0 mL, 12.98 mmol, 95.37 eq) in methylene chloride (4 mL) was stirred at 25° C. for 1 h. The resulting solution was concentrated under reduced pressure to give 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (60.0 mg, 0.11 mmol, 77.3% yield). MS [M+H]⁺: 525.0

### Step 4: tert-butyl (1R,5S)-6-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-6-fluoro-3-azabicyclo[3.1.0]hexane-3-carboxylate

To a solution of 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (50.0 mg, 0.1 mmol, 1.0 eq) in methylene chloride (3 mL) were added (1R,5S)-3-tert-butoxycarbonyl-6-fluoro-3-azabicyclo[3.1.0]hexane-6-carboxylic acid (46.76 mg, 0.19 mmol, 2.0 eq), 1,3,5,2,4,6-trioxatriphosphorinane, 2,4,6-tributyl-, 2,4,6-trioxide (72.5 mg, 0.19 mmol, 2.0 eq) and N,N-diisopropylethylamine (36.96 mg, 0.29 mmol, 3.0 eq). The resulting mixture was stirred at 25° C. for 1 h. The solvent was removed under reduced pressure, and the residue was diluted with EtOAc (5 mL), washed with H₂O (3×5 mL), dried over Na₂SO₄ and concentrated under reduced pressure to give tert-butyl (1R,5S)-6-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-6-fluoro-3-azabicyclo[3.1.0]hexane-3-carboxylate (65.0 mg, 0.09 mmol, 39.9% yield). MS [M+H]⁺: 752.2

### Step 5: 5-amino-1-[2-(difluoromethyl)-3-fluoro-4-(fluoromethoxy)phenyl]-N-[3-fluoro-4-[4-[(1R,5S)-6-fluoro-3-azabicyclo[3.1.0]hexane-6-carbonyl]piperazine-1-carbonyl]phenyl]imidazole-4-carboxamide; 1:1 2,2,2-trifluoroacetic acid

A solution of tert-butyl (1R,5S)-6-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-6-fluoro-3-azabicyclo[3.1.0]hexane-3-carboxylate (65.0 mg, 0.09 mmol, 1.0 eq) and trifluoroacetic acid (1.0 mL, 12.98 mmol, 150.1 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 30-60) to afford 5-amino-1-[2-(difluoromethyl)-3-fluoro-4-(fluoromethoxy)phenyl]-N-[3-fluoro-4-[4-[(1R,5S)-6-fluoro-3-azabicyclo[3.1.0]hexane-6-carbonyl]piperazine-1-carbonyl]phenyl]imidazole-4-carboxamide;1:1 2,2,2-trifluoroacetic acid (34.7 mg, 0.05 mmol, 58.8% yield). MS [M+H]⁺: 652.1

### Compound 206

### 5-amino-N-[3-chloro-4-[4-[(1R,5S,6r)-6-fluoro-3-azabicyclo[3.1.0]hexane-6-carbonyl]piperazine-1-carbonyl]phenyl]-1-[1-cyclobutyl-3-(trifluoromethyl)pyrazol-4-yl]imidazole-4-carboxamide; 1:1 2,2,2-trifluoroacetic acid

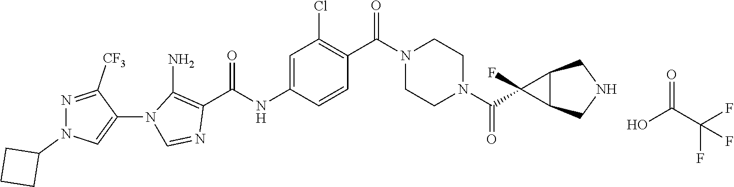 [SMILES: Nc1c(C(=O)Nc2ccc(C(=O)N3CCN(C(=O)[C@@]4(F)[C@@H]5CNC[C@@H]54)CC3)c(Cl)c2)ncn1-c1cn(C2CCC2)nc1C(F)(F)F ; O=C(O)C(F)(F)F]

### Step 1: tert-butyl 4-[4-[[5-amino-1-[1-[(4-methoxyphenyl)methyl]-3-(trifluoromethyl)pyrazol-4-yl]imidazole-4-carbonyl]amino]-2-chlorobenzoyl]piperazine-1-carboxylate

To a solution of tert-butyl 4-(4-amino-2-chlorobenzoyl)piperazine-1-carboxylate (1700.12 mg, 5.0 mmol, 1.0 eq) and pyridine (6.07 mL, 75.05 mmol, 15.0 eq) in methylene chloride (10 mL) was added a suspension of 5-amino-1-[1-[(4-methoxyphenyl)methyl]-3-(trifluoromethyl)pyrazol-4-yl]imidazole-4-carbonyl chloride (2000.0 mg, 5.0 mmol, 1.0 eq) in methylene chloride (10 mL) at 25° C. The mixture was stirred at 25° C. for 1 h under N₂. The resulting solution was evaporated under reduced pressure. The residue was purified by column chromatography (silica gel, 0 to 10% MeOH in methylene chloride) to give tert-butyl 4-[4-[[5-amino-1-[1-[(4-methoxyphenyl)methyl]-3-(trifluoromethyl)pyrazol-4-yl]imidazole-4-carbonyl]amino]-2-chlorobenzoyl]piperazine-1-carboxylate (2500.0 mg, 3.56 mmol, 71.1% yield). MS [M+H]⁺: 702.9

### Step 2: 5-amino-N-[3-chloro-4-(piperazine-1-carbonyl)phenyl]-1-[3-(trifluoromethyl)-1H-pyrazol-4-yl]imidazole-4-carboxamide

A solution of tert-butyl 4-[4-[[5-amino-1-[1-[(4-methoxyphenyl)methyl]-3-(trifluoromethyl)pyrazol-4-yl]imidazole-4-carbonyl]amino]-2-chlorobenzoyl]piperazine-1-carboxylate (3000.0 mg, 4.27 mmol, 1.0 eq) in trifluoroacetic acid (28.16 mL, 365.54 mmol, 85.67 eq) was stirred at 70° C. for 9 h. The resulting solution was concentrated under reduced pressure to give 5-amino-N-[3-chloro-4-(piperazine-1-carbonyl)phenyl]-1-[3-(trifluoromethyl)-1H-pyrazol-4-yl]imidazole-4-carboxamide (1000.0 mg, 2.07 mmol, 48.5% yield). MS [M+H]⁺: 483.0

### Step 3: tert-butyl (1R,5S)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-6-fluoro-3-azabicyclo[3.1.0]hexane-3-carboxylate

To a solution of (1R,5S)-3-tert-butoxycarbonyl-6-fluoro-3-azabicyclo[3.1.0]hexane-6-carboxylic acid (507.92 mg, 2.07 mmol, 1.0 eq) in DMF (12 mL) was added 2-(7-azabenzotriazol-1-yl)-N,N,N′,N′-tetramethyluronium hexafluorophosphate (1574.98 mg, 4.14 mmol, 2.0 eq), triethylamine (628.7 mg, 6.21 mmol, 3.0 eq) and 5-amino-N-[3-chloro-4-(piperazine-1-carbonyl)phenyl]-1-[3-(trifluoromethyl)-1H-pyrazol-4-yl]imidazole-4-carboxamide (1000.0 mg, 2.07 mmol, 1.0 eq) was stirred at 25° C. for 2 h. After concentration to dryness, the residue was taken up in EtOAc (30 mL) and washed with 2×30 mL water followed by 30 mL brine. The organics were then separated and dried (MgSO₄) before concentrating to dryness. The crude was then purified by flash column chromatography eluting with 60% EtOAc in petroleum ether to give tert-butyl (1R,5S)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-6-fluoro-3-azabicyclo[3.1.0]hexane-3-carboxylate (730.0 mg, 1.03 mmol, 49.6% yield). MS [M−tBu+H]⁺: 654.0

### Step 4: tert-butyl (1R,5S)-6-[4-[4-[[5-amino-1-[1-cyclobutyl-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate

To a solution of tert-butyl (1R,5S)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-6-fluoro-3-azabicyclo[3.1.0]hexane-3-carboxylate (70.0 mg, 0.1 mmol, 1.0 eq) in MeCN (2 mL) were added bromocyclobutane (66.54 mg, 0.49 mmol, 5.0 eq) and potassium carbonate (27.25 mg, 0.2 mmol, 2.0 eq). The resulting mixture was stirred at 80° C. for 15 h. The resulting solution was diluted with 10 ml of water and then extracted with 3×10 mL of EtOAc. The organic layers were combined, dried and concentrated under reduced pressure. The residue was applied on a silica gel column and eluted with 94:6 methylene chloride:MeOH to give tert-butyl (1R,5S)-6-[4-[4-[[5-amino-1-[1-cyclobutyl-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate (62.0 mg, 0.08 mmol, 82.3% yield). MS [M−Boc+H]⁺: 664.0

### Step 5: 5-amino-N-[3-chloro-4-[4-[(1R,5S)-6-fluoro-3-azabicyclo[3.1.0]hexane-6-carbonyl]piperazine-1-carbonyl]phenyl]-1-[1-cyclobutyl-3-(trifluoromethyl)pyrazol-4-yl]imidazole-4-carboxamide; 1:1 2,2,2-trifluoroacetic acid

To a solution of tert-butyl (1R,5S)-6-[4-[4-[[5-amino-1-[1-cyclobutyl-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate (62.0 mg, 0.08 mmol, 1.0 eq) in methylene chloride (2 mL) was added trifluoroacetic acid (1.41 mL, 18.4 mmol, 226.8 eq). The resulting mixture was stirred at 25° C. for 1 h. After concentration under reduced pressure, the residue was purified by SFC [chiral Pak-OJ, CO₂-MeOH(DEtOAc), 55˜70%] and prep-HPLC [Gemini-C18 150×21.2 mm, 5 μm, MeCN— H₂O (0.1% TFA), 40%˜60%] to give 5-amino-N-[3-chloro-4-[4-[(1R,5S)-6-fluoro-3-azabicyclo[3.1.0]hexane-6-carbonyl]piperazine-1-carbonyl]phenyl]-1-[1-cyclobutyl-3-(trifluoromethyl)pyrazol-4-yl]imidazole-4-carboxamide; 1:1 2,2,2-trifluoroacetic acid (18.0 mg, 0.02 mmol, 28.2% yield). MS [M+H]⁺: 664.0

### Compound 207

### 5-amino-N-[3-chloro-4-[4-[(1R,5S,6r)-6-fluoro-3-azabicyclo[3.1.0]hexane-6-carbonyl]piperazine-1-carbonyl]phenyl]-1-[2-(difluoromethyl)-3-fluoro-4-(fluoromethoxy)phenyl]imidazole-4-carboxamide; 1:1 2,2,2-trifluoroacetic acid

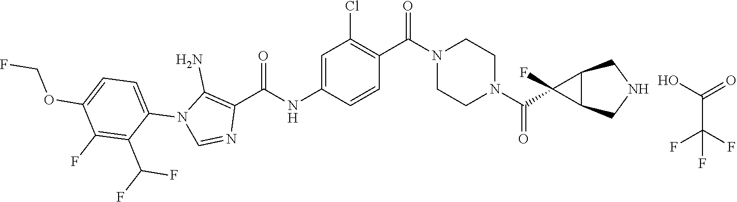

### Step 1: 5-amino-1-[2-(difluoromethyl)-3-fluoro-4-(fluoromethoxy)phenyl]imidazole-4-carbonyl chloride

A solution of 5-amino-1-[2-(difluoromethyl)-3-fluoro-4-(fluoromethoxy)phenyl]imidazole-4-carboxylic acid (400.0 mg, 1.25 mmol, 1.0 eq) in thionyl chloride (4.78 mL, 65.86 mmol, 52.56 eq) was stirred at 25° C. for 1 h. The resulting solution was concentrated under reduced pressure to give 5-amino-1-[2-(difluoromethyl)-3-fluoro-4-(fluoromethoxy)phenyl]imidazole-4-carbonyl chloride (400.0 mg, 1.18 mmol, 94.5% yield). MS [(M−Cl+OCH₃)+H]⁺: 334.0

### Step 2: tert-butyl 4-[4-[[5-amino-1-[2-(difluoromethyl)-3-fluoro-4-(fluoromethoxy)phenyl]imidazole-4-carbonyl]amino]-2-chlorobenzoyl]piperazine-1-carboxylate

To a solution of tert-butyl 4-(4-amino-2-chlorobenzoyl)piperazine-1-carboxylate (402.56 mg, 1.18 mmol, 1.0 eq) and pyridine (1.92 mL, 23.69 mmol, 20.0 eq) in methylene chloride (2 mL) was added a suspension of 5-amino-1-[2-(difluoromethyl)-3-fluoro-4-(fluoromethoxy)phenyl]imidazole-4-carbonyl chloride (400.0 mg, 1.18 mmol, 1.0 eq) in methylene chloride (1 mL) at 25° C. The reaction was concentrated to dryness and the residue was taken up in methylene chloride (10 mL). The organics were washed with 2×5 mL water followed by 5 mL brine. The organics were then separated and dried (Na₂SO₄) before concentrating to dryness. The crude was then purified by flash column chromatography eluting with 4% MeOH in methylene chloride to give tert-butyl 4-[4-[[5-amino-1-[2-(difluoromethyl)-3-fluoro-4-(fluoromethoxy)phenyl]imidazole-4-carbonyl]amino]-2-chlorobenzoyl]piperazine-1-carboxylate (600.0 mg, 0.94 mmol, 79.0% yield). MS [M+H]⁺: 640.9

### Step 3: 5-amino-N-[3-chloro-4-(piperazine-1-carbonyl)phenyl]-1-[2-(difluoromethyl)-3-fluoro-4-(fluoromethoxy)phenyl]imidazole-4-carboxamide

To a solution of tert-butyl 4-[4-[[5-amino-1-[2-(difluoromethyl)-3-fluoro-4-(fluoromethoxy)phenyl]imidazole-4-carbonyl]amino]-2-chlorobenzoyl]piperazine-1-carboxylate (600.0 mg, 0.94 mmol, 1.0 eq) and trifluoroacetic acid (1.0 mL, 12.98 mmol, 13.87 eq) in methylene chloride (3 mL) was stirred at 25° C. for 1 h. The resulting solution was concentrated under reduced pressure to give 5-amino-N-[3-chloro-4-(piperazine-1-carbonyl)phenyl]-1-[2-(difluoromethyl)-3-fluoro-4-(fluoromethoxy)phenyl]imidazole-4-carboxamide (400.0 mg, 0.74 mmol, 77.6% yield). MS [M+H]⁺: 541.0

### Step 4: tert-butyl (1R,5S)-6-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-6-fluoro-3-azabicyclo[3.1.0]hexane-3-carboxylate

To a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide (50.0 mg, 0.09 mmol, 1.0 eq) and (1R,5S)-3-tert-butoxycarbonyl-6-fluoro-3-azabicyclo[3.1.0]hexane-6-carboxylic acid (22.67 mg, 0.09 mmol, 1.0 eq) in methylene chloride (1 mL) was added 2-(7-azabenzotriazol-1-yl)-N,N,N′,N′-tetramethyluronium hexafluorophosphate (70.3 mg, 0.18 mmol, 2.0 eq) and triethylamine (0.04 mL, 0.28 mmol, 3.0 eq). The reaction mixture was stirred at 25° C. for 2 h. The reaction was quenched with water (25 mL) and extracted with ethyl acetate (3×20 mL). The combined organic phases were dried over anhydrous sodium sulfate and concentrated under reduced pressure. The residue was purified by flash column to give tert-butyl (1R,5S)-6-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-6-fluoro-3-azabicyclo[3.1.0]hexane-3-carboxylate (50.0 mg, 0.07 mmol, 68.6% yield). MS [M+H]⁺: 767.9.

### Step 5: 5-amino-N-[3-chloro-4-[4-[(1R,5S)-6-fluoro-3-azabicyclo[3.1.0]hexane-6-carbonyl]piperazine-1-carbonyl]phenyl]-1-[2-(difluoromethyl)-3-fluoro-4-(fluoromethoxy)phenyl]imidazole-4-carboxamide; 1:1 2,2,2-trifluoroacetic acid

To a solution of tert-butyl (1R,5S)-6-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-6-fluoro-3-azabicyclo[3.1.0]hexane-3-carboxylate (50.0 mg, 0.07 mmol, 1.0 eq) and trifluoroacetic acid (1.0 mL, 12.98 mmol, 199.41 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA), 25%-40%) to give 5-amino-N-[3-chloro-4-[4-[(1R,5S)-6-fluoro-3-azabicyclo[3.1.0]hexane-6-carbonyl]piperazine-1-carbonyl]phenyl]-1-[2-(difluoromethyl)-3-fluoro-4-(fluoromethoxy)phenyl]imidazole-4-carboxamide; 1:1 2,2,2-trifluoroacetic acid (19.3 mg, 0.02 mmol, 37.3% yield). MS [M+H]⁺: 668.0

### Compound 208

### 5-amino-1-(1-(2-chloroallyl)-3-(trifluoromethyl)-1H-pyrazol-4-yl)-N-(3-fluoro-4-(4-((1R,5S,6r)-6-fluoro-3-azabicyclo[3.1.0]hexane-6-carbonyl)piperazine-1-carbonyl)phenyl)-1H-imidazole-4-carboxamide;2,2,2-trifluoroacetic acid

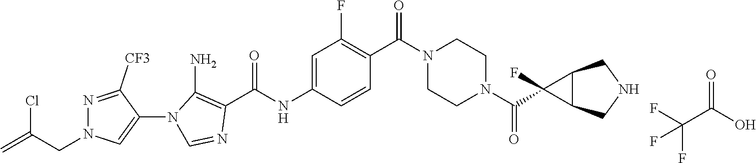

The intermediate 1 was prepared as intermediate

### Step 1: tert-butyl (1R,5S,6r)-6-(4-(4-(5-amino-1-(3-(trifluoromethyl)-1H-pyrazol-4-yl)-1H-imidazole-4-carboxamido)-2-fluorobenzoyl)piperazine-1-carbonyl)-6-fluoro-3-azabicyclo[3.1.0]hexane-3-carboxylate

1,3,5,2,4,6-Trioxatriphosphorinane, 2,4,6-tributyl-2,4,6-trioxide (556.15 mg, 0.77 mmol, 1.8 eq)(50% in EtOAc) was added to a solution of 5-amino-N-[3-fluoro-4-(piperazine-1-carbonyl)phenyl]-1-[3-(trifluoromethyl)-1H-pyrazol-4-yl]imidazole-4-carboxamide (200.0 mg, 0.43 mmol, 1.0 eq), (1R,5S)-3-tert-butoxycarbonyl-6-fluoro-3-azabicyclo[3.1.0]hexane-6-carboxylic acid (136.72 mg, 0.56 mmol, 1.3 eq) and N,N-diisopropylethylamine (221.68 mg, 1.72 mmol, 4.0 eq) in methylene chloride (10 mL) dropwise, and the mixture was stirred at 25° C. for 1 h. The reaction was concentrated to dryness and the residue was taken up in EtOAc (30 mL). The organics were washed with 30 mL water followed by 30 mL brine. The organics were then separated and dried (Na₂SO₄) before concentrating to dryness. The crude was then purified by flash column chromatography eluting with 0˜10% MeOH in methylene chloride to afford tert-butyl (1R,5S)-6-[4-[4-[[5-amino-1-[3-(trifluoromethyl)-1H-pyrazol-4-yl]imidazole-4-carbonyl]amino]-2-fluorobenzoyl]piperazine-1-carbonyl]-6-fluoro-3-azabicyclo[3.1.0]hexane-3-carboxylate (220.0 mg, 0.32 mmol, 67.6% yield). MS [M+H−tBu]⁺: 638.3

### Step 2: tert-butyl (1R,5S,6r)-6-(4-(4-(5-amino-1-(1-(2-chloroallyl)-3-(trifluoromethyl)-1H-pyrazol-4-yl)-1H-imidazole-4-carboxamido)-2-fluorobenzoyl)piperazine-1-carbonyl)-6-fluoro-3-azabicyclo[3.1.0]hexane-3-carboxylate

Potassium carbonate (71.73 mg, 0.52 mmol, 2.0 eq) was added to a solution of tert-butyl (1R,5S)-6-[4-[4-[[5-amino-1-[3-(trifluoromethyl)-1H-pyrazol-4-yl]imidazole-4-carbonyl]amino]-2-fluorobenzoyl]piperazine-1-carbonyl]-6-fluoro-3-azabicyclo[3.1.0]hexane-3-carboxylate (180.0 mg, 0.26 mmol, 1.0 eq) and 2-chloroallyl 4-methylbenzenesulfonate (192.06 mg, 0.78 mmol, 3.0 eq) in DMF (5 mL), The mixture was stirred at 25° C. for 3 h. The reaction was taken up in EtOAc (20 mL) and the organics washed with 20 mL water followed by 20 mL brine. The organics were then separated and dried (Na₂SO₄) before concentrating to dryness. The crude was then purified by flash column chromatography eluting with 0˜10% MeOH in methylene chloride to afford tert-butyl (1R,5S)-6-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]-6-fluoro-3-azabicyclo[3.1.0]hexane-3-carboxylate (150.0 mg, 0.2 mmol, 75.2% yield). MS [M+H−t−Bu]⁺: 712.2

### Step 3: 5-amino-1-(1-(2-chloroallyl)-3-(trifluoromethyl)-1H-pyrazol-4-yl)-N-(3-fluoro-4-(4-((1R,5S,6r)-6-fluoro-3-azabicyclo[3.1.0]hexane-6-carbonyl)piperazine-1-carbonyl)phenyl)-1H-imidazole-4-carboxamide;2,2,2-trifluoroacetic acid

Trifluoroacetic acid (1 mL) was added to a solution of tert-butyl (1R,5S)-6-[4-[4-[[5-amino-1-[1-(2-chloroallyl)-3-(trifluoromethyl)pyrazol-4-yl]imidazole-4-carbonyl]amino]-2-fluorobenzoyl]piperazine-1-carbonyl]-6-fluoro-3-azabicyclo[3.1.0]hexane-3-carboxylate (140.0 mg, 0.18 mmol, 1.0 eq) in methylene chloride (5 mL). The mixture was stirred at 25° C. for 1 h. The mixture was concentrated then purified by prep-HPLC: column Gemini-C18 150×21.2 m, 5 μm MeCN-H₂O (0.1% TFA) to afford 5-amino-1-[1-(2-chloroallyl)-3-(trifluoromethyl)pyrazol-4-yl]-N-[3-fluoro-4-[4-[(1R,5S)-6-fluoro-3-azabicyclo[3.1.0]hexane-6-carbonyl]piperazine-1-carbonyl]phenyl]imidazole-4-carboxamide;2,2,2-trifluoroacetic acid (109.5 mg, 0.14 mmol, 76.7% yield). MS [M+H]⁺: 668.3

### Compound 209

### 5-amino-N-[3-chloro-4-[4-[(1R,5S,6r)-6-fluoro-3-azabicyclo[3.1.0]hexane-6-carbonyl]piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid

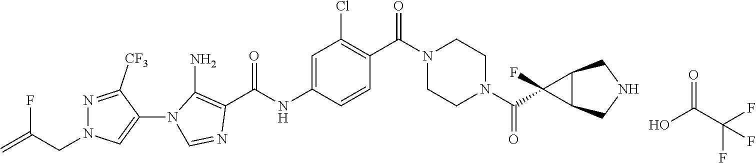 [SMILES: C=C(F)Cn1cc(-n2cnc(C(=O)Nc3ccc(C(=O)N4CCN(C(=O)[C@@]5(F)[C@@H]6CNC[C@@H]65)CC4)c(Cl)c3)c2N)c(C(F)(F)F)n1 ; O=C(O)C(F)(F)F]

### Step 1: tert-butyl 4-[4-[[5-amino-1-[1-(2-fluoroallyl)-3-(trifluoromethyl)pyrazol-4-yl]imidazole-4-carbonyl]amino]-2-chlorobenzoyl]piperazine-1-carboxylate

To a solution of tert-butyl 4-(4-amino-2-chlorobenzoyl)piperazine-1-carboxylate (119.76 mg, 0.35 mmol, 0.7 eq) in methylene chloride (2 mL) were added pyridine (0.41 mL, 5.03 mmol, 10.0 eq) and 5-amino-1-[1-(2-fluoroallyl)-3-(trifluoromethyl)pyrazol-4-yl]imidazole-4 carbonyl chloride (170.0 mg, 0.5 mmol, 1.0 eq) under N₂ atmosphere. The resulting mixture was stirred at 25° C. for 1 h before it was concentrated and diluted with EtOAc (30 mL). The organic solution was washed with H₂O (3×25 mL), dried over Na₂SO₄ and concentrated under reduced pressure to give tert-butyl 4-[4-[[5-amino-1-[1-(2-fluoroallyl)-3-(trifluoromethyl)pyrazol-4-yl]imidazole-4-carbonyl]amino]-2-chlorobenzoyl]piperazine-1-carboxylate (280.0 mg, 0.44 mmol, 37.3% yield). MS [M+H]⁺: 641.1

Step 2: 5-amino-N-[3-chloro-4-(piperazine-1-carbonyl)phenyl]-1-[1-(2-fluoroallyl)-3-(trifluoromethyl)pyrazol-4-yl]imidazole-4-carboxamide; 1:1 2,2,2-trifluoroacetic acid

To a solution of tert-butyl 4-[4-[[5-amino-1-[1-(2-fluoroallyl)-3-(trifluoromethyl)pyrazol-4-yl]imidazole-4-carbonyl]amino]-2-chlorobenzoyl]piperazine-1-carboxylate (280.0 mg, 0.44 mmol, 1.0 eq) in methylene chloride (3 mL) was added trifluoroacetic acid (1.0 mL, 13.07 mmol, 29.92 eq) under N₂ atmosphere. The resulting mixture was stirred at 25° C. for 1 h. Concentrated under reduced pressure to give 5-amino-N-[3-chloro-4-(piperazine-1-carbonyl)phenyl]-1-[1-(2-fluoroallyl)-3-(trifluoromethyl)pyrazol-4-yl]imidazole-4-carboxamide; 1:1 2,2,2-trifluoroacetic acid (245.0 mg, 0.37 mmol, 34.3% yield). MS [M+H]⁺: 541.0

### Step 3: tert-butyl(1R,5S)-6-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate

To a solution of 5-amino-N-[3-chloro-4-(piperazine-1-carbonyl)phenyl]-1-[1-(2-fluoro-allyl)-3-(trifluoromethyl)pyrazol-4-yl]imidazole-4-carboxamide;2,2,2-trifluoroacetic acid (245.0 mg, 0.37 mmol, 1.0 eq) in methylene chloride (6 mL) were added (1R,5S)-3-tert-butoxycarbonyl-6-fluoro-3-azabicyclo[3.1.0]hexane-6-carboxylic acid (119.27 mg, 0.49 mmol, 1.3 eq), N,N-diisopropylethylamine (145.04 mg, 1.12 mmol, 3.0 eq) and 1,3,5,2,4,6-trioxa-triphosphorinane, 2,4,6-tributyl-2,4,6-trioxide (404.3 mg, 0.56 mmol, 1.5 eq) under N₂ atmosphere. The resulting mixture was stirred at 25° C. for 1 h and then diluted with EtOAc (30 mL). The resulting solution was washed with H₂O (3×25 mL), dried over Na₂SO₄ and concentrated under reduced pressure. The residue was purified by flash column chromatography (94:6 methylene chloridemia methyl alcohol) to give tert-butyl(1R,5S)-6-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate (254.0 mg, 0.33 mmol, 34.5% yield). MS [M-Boc+H]⁺: 668.0

### Step 4: 5-amino-N-[3-chloro-4-[4-[(1R,5S)-6-fluoro-3-azabicyclo[3.1.0]hexane-6-carbonyl]piperazine-1-carbonyl]phenyl]-1-[1-(2-fluoroallyl)-3-(trifluoromethyl)pyrazol-4-yl]imidazole-4-carboxamide; 1:1 2,2,2-trifluoroacetic acid

To a solution of tert-butyl(1R,5S)-6-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate (254.0 mg, 0.13 mmol, 1.0 eq) in methylene chloride (2 mL) was added trifluoroacetic acid (1.0 mL, 13 mmol, 100 eq). The resulting mixture was stirred at 25° C. for 1 h. After concentration under reduced pressure, the residue was purified by prep-HPLC [Gemini-C18 150×21.2 mm, 5 μm, MeCN— H₂O (0.1% TFA), 25%˜35%] to give 5-amino-N-[3-chloro-4-[4-[(1R,5S)-6-fluoro-3-azabicyclo[3.1.0]hexane-6-carbonyl]piperazine-1-carbonyl]phenyl]-1-[1-(2-fluoroallyl)-3-(trifluoromethyl)pyrazol-4-yl]imidazole-4-carboxamide;2,2,2-trifluoroacetic acid (67.5 mg, 0.09 mmol, 63.6% yield). MS [M+H]⁺: 668.0

### Compound 210

### 5-amino-1-[1-(2-chloroallyl)-3-(trifluoromethyl)pyrazol-4-yl]-N-[3-chloro-4-[4-[(1S,5R,6r)-6-fluoro-3-azabicyclo[3.1.0]hexane-6-carbonyl]piperazine-1-carbonyl]phenyl]imidazole-4-carboxamide;2,2,2-trifluoroacetic acid

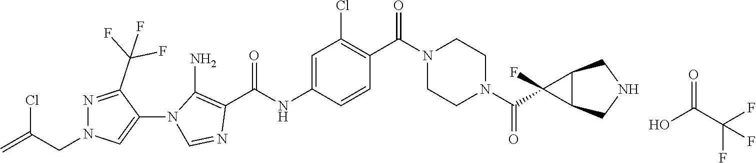 [SMILES: C=C(Cl)Cn1cc(-n2cnc(C(=O)Nc3ccc(C(=O)N4CCN(C(=O)[C@@]5(F)[C@@H]6CNC[C@@H]65)CC4)c(Cl)c3)c2N)c(C(F)(F)F)n1 ; O=C(O)C(F)(F)F]

### Step 1: tert-butyl(1S,5R)-6-[4-[4-[[5-amino-1-[1-(2-chloroallyl)-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate

A solution of tert-butyl(1S,5R)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-6-fluoro-3-azabicyclo[3.1.0]hexane-3-carboxylate (50.0 mg, 0.07 mmol, 1.0 eq), 2-chloroallyl 4-methylbenzenesulfonate (173.72 mg, 0.7 mmol, 10.0 eq) and potassium carbonate (97.32 mg, 0.7 mmol, 10.0 eq) in DMF (1 mL) was stirred at 25° C. for 1 h. The resulting solution was concentrated under reduced pressure. The reaction was concentrated to dryness to afford crude product. The crude product was then purified by flash column chromatography eluting with 10:1 methylene chloridemia to give tert-butyl(1S,5R)-6-[4-[4-[[5-amino-1-[1-(2-chloroallyl)-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate (40.0 mg, 0.05 mmol, 72.4% yield). MS [M+H]⁺: 784.2

### Step 2: 5-amino-1-[1-(2-chloroallyl)-3-(trifluoromethyl)pyrazol-4-yl]-N-[3-chloro-4-[4-[(1S,5R)-6-fluoro-3-azabicyclo[3.1.0]hexane-6-carbonyl]piperazine-1-carbonyl]phenyl]imidazole-4-carboxamide;2,2,2-trifluoroacetic acid

A solution of tert-butyl(1S,5R)-6-[4-[4-[[5-amino-1-[1-(2-chloroallyl)-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate (50.0 mg, 0.06 mmol, 1.0 eq) and trifluoroacetic acid (7.27 mg, 0.06 mmol, 1.0 eq) in methylene chloride (1 mL) was stirred at 25° C. for 1 h. The resulting solution was concentrated under reduced pressure. The crude product was purified by Prep-HPLC (Gemini-C18 150×21.2 mm, 5 μm, MeCN-H₂O (0.1% TFA), 15-45) to give 5-amino-1-[1-(2-chloroallyl)-3-(trifluoromethyl)pyrazol-4-yl]-N-[3-chloro-4-[4-[(1S,5R)-6-fluoro-3-azabicyclo[3.1.0]hexane-6-carbonyl]piperazine-1-carbonyl]phenyl]imidazole-4-carboxamide;2,2,2-trifluoroacetic acid (24.2 mg, 0.03 mmol, 55.2% yield). MS [M+H]⁺: 683.9

### Compound 211

### 5-amino-N-[3-chloro-4-[4-fluoro-4-(piperazine-1-carbonyl)piperidine-1-carbonyl]phenyl]-1-[2-(difluoromethyl)-3-fluoro-4-(fluoromethoxy)phenyl]imidazole-4-carboxamide; 1:1 2,2,2-trifluoroacetic acid

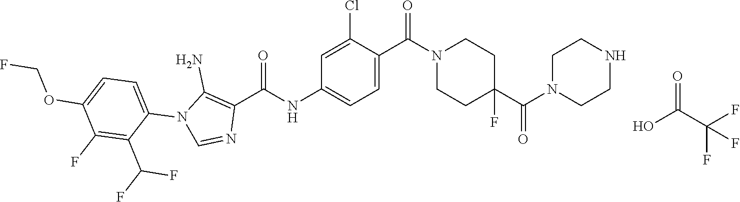 [SMILES: Nc1c(C(=O)Nc2ccc(C(=O)N3CCC(F)(C(=O)N4CCNCC4)CC3)c(Cl)c2)ncn1-c1ccc(OCF)c(F)c1C(F)F ; O=C(O)C(F)(F)F]

### Step 1: ethyl 1-(2-chloro-4-nitrobenzoyl)-4-fluoropiperidine-4-carboxylate

To a solution of 2-chloro-4-nitrobenzoic acid (300.0 mg, 1.49 mmol, 1.0 eq) in methylene chloride (5 mL) were added ethyl 4-fluoropiperidine-4-carboxylate;hydrochloride (378.04 mg, 1.79 mmol, 1.2 eq), N-butylphosphonic anhydride (2144.83 mg, 2.98 mmol, 2.0 eq) and N,N-diisopropylethylamine (0.78 mL, 4.47 mmol, 3.0 eq). The resulting mixture was stirred at 25° C. for 1 h. The solvent was removed under reduced pressure. The residue was diluted with EtOAc (5 mL), washed with H₂O (3×5 mL), dried over Na₂SO₄ and concentrated under reduced pressure to give ethyl 1-(2-chloro-4-nitrobenzoyl)-4-fluoropiperidine-4-carboxylate (420.0 mg, 1.17 mmol, 78.7% yield). MS [M-Boc+H]⁺: 298.0

### Step 2: 1-(2-chloro-4-nitrobenzoyl)-4-fluoropiperidine-4-carboxylic acid

Ethyl 1-(2-chloro-4-nitrobenzoyl)-4-fluoropiperidine-4-carboxylate (420.0 mg, 1.17 mmol, 1.0 eq) was dissolved in isopropyl alcohol (10 mL). A solution of sodium hydroxide (234.13 mg, 5.85 mmol, 5.0 eq) in water (5 mL) was added dropwise at 25° C. and stirred at 50° C. for 2 h. The solvent was then evaporated. The residue was diluted with water (10 mL), and the solution was extracted with ethyl acetate (2×10 mL). The aqueous layer was acidified to pH 5 with HCl (2 M), and the product was collected by filtration to give 1-(2-chloro-4-nitrobenzoyl)-4-fluoropiperidine-4-carboxylic acid (310.0 mg, 0.94 mmol, 80.1% yield). MS [M+H]⁺: 331.0

### Step 3: tert-butyl 4-[1-(2-chloro-4-nitrobenzoyl)-4-fluoropiperidine-4-carbonyl]piperazine-1-carboxylate

To a solution of 1-(2-chloro-4-nitrobenzoyl)-4-fluoropiperidine-4-carboxylic acid (310.0 mg, 0.94 mmol, 1.0 eq) in methylene chloride (5 mL) were added 1-BOC-piperazine (209.51 mg, 1.12 mmol, 1.2 eq), N-butylphosphonic anhydride (1350.84 mg, 1.87 mmol, 2.0 eq) and N,N-diisopropylethylamine (0.49 mL, 2.81 mmol, 3.0 eq). The resulting mixture was stirred at 25° C. for 1 h. The solvent was removed under reduced pressure. The residue was diluted with EtOAc (5 mL) and washed with H₂O (3×5 mL), dried over Na₂SO₄ and concentrated under reduced pressure to give tert-butyl 4-[1-(2-chloro-4-nitrobenzoyl)-4-fluoropiperidine-4-carbonyl]piperazine-1-carboxylate (390.0 mg, 0.78 mmol, 83.4% yield). MS [M-Boc+H]⁺: 443.0

### Step 4: tert-butyl 4-[1-(4-amino-2-chlorobenzoyl)-4-fluoropiperidine-4-carbonyl]piperazine-1-carboxylate

A mixture of tert-butyl 4-[1-(2-chloro-4-nitrobenzoyl)-4-fluoropiperidine-4-carbonyl]piperazine-1-carboxylate (390.0 mg, 0.78 mmol, 1.0 eq), ammonium chloride (209.05 mg, 3.91 mmol, 5.0 eq) and iron powder (218.86 mg, 3.91 mmol, 5.0 eq) in 1:1 ethanol (10 mL) was reacted at 60° C. for 4 h. The reaction mixture was quenched by the addition of the saturated aqueous NaCl. After quenching the reaction, the reaction mixture was poured into separatory funnel and separated. The residue was partitioned with ethyl acetate (100 mL). The organic layer was washed with water, dried (Na₂SO₄) and evaporated to dryness to afford tert-butyl 4-[1-(4-amino-2-chlorobenzoyl)-4-fluoropiperidine-4-carbonyl]piperazine-1-carboxylate (270.0 mg, 0.58 mmol, 73.7% yield). MS [M+H]⁺: 469.0

### Step 5: 5-amino-1-[2-(difluoromethyl)-3-fluoro-4-(fluoromethoxy)phenyl]imidazole-4-carboxylic acid

Ethyl 5-amino-1-[2-(difluoromethyl)-3-fluoro-4-(fluoromethoxy)phenyl]imidazole-4-carboxylate (500.0 mg, 1.44 mmol, 1.0 eq) was dissolved in isopropyl alcohol (10 mL). A solution of sodium hydroxide (287.94 mg, 7.2 mmol, 5.0 eq) in water (5 mL) was added dropwise at 25° C. and stirred at 50° C. for 2 h. Solvent was evaporated. The residue was diluted with water (10 mL), and the solution was extracted with ethyl acetate (2×10 mL). The aqueous layer was acidified to pH 5 with HCl (2 M), and the product was collected by filtration to give 5-amino-1-[2-(difluoromethyl)-3-fluoro-4-(fluoromethoxy)phenyl]imidazole-4-carboxylic acid (190.0 mg, 0.6 mmol, 41.3% yield). MS [M+H]⁺: 320.0

### Step 6: 5-amino-1-[2-(difluoromethyl)-3-fluoro-4-(fluoromethoxy)phenyl]imidazole-4-carbonyl chloride

5-Amino-1-[2-(difluoromethyl)-3-fluoro-4-(fluoromethoxy)phenyl]imidazole-4-carboxylic acid (70.0 mg, 0.22 mmol, 1.0 eq) was dissolved in thionyl chloride (3.0 mL, 41.35 mmol, 188.59 eq) at 25° C. The reaction mixture was stirred at 25° C. for 1 h under N₂. The resulting solution was evaporated to dryness to give 5-amino-1-[2-(difluoromethyl)-3-fluoro-4-(fluoromethoxy)phenyl]imidazole-4-carbonyl chloride (70.0 mg, 0.21 mmol, 61.4% yield), which was used without further purification. MS [M−Cl+OMe+H]⁺: 334.0

### Step 7: tert-butyl 4-[1-[4-[[5-amino-1-[2-(difluoromethyl)-3-fluoro-4-(fluoromethoxy)phenyl]imidazole-4-carbonyl]amino]-2-chlorobenzoyl]-4-fluoropiperidine-4-carbonyl]piperazine-1-carboxylate

A solution of tert-butyl 4-[1-(4-amino-2-chlorobenzoyl)-4-fluoropiperidine-4-carbonyl]piperazine-1-carboxylate (77.77 mg, 0.17 mmol, 0.8 eq) and pyridine (0.34 mL, 4.15 mmol, 20.0 eq) in methylene chloride (2 mL) was stirred at 25° C. Next 5-amino-1-[2-(difluoromethyl)-3-fluoro-4-(fluoromethoxy)phenyl]imidazole-4-carbonyl chloride (70.0 mg, 0.21 mmol, 1.0 eq) in methylene chloride (2 mL) was added, and the mixture was stirred at 25° C. for 1 h under N₂. The resulting solution was concentrated under reduced pressure. The residue was applied on a silica gel column and eluted with 10:1 methylene chloridemia to give tert-butyl 4-[1-[4-[[5-amino-1-[2-(difluoromethyl)-3-fluoro-4-(fluoromethoxy)phenyl]imidazole-4-carbonyl]amino]-2-chlorobenzoyl]-4-fluoropiperidine-4-carbonyl]piperazine-1-carboxylate (90.0 mg, 0.12 mmol, 39.5% yield). MS [M+H]⁺: 770.0

### Step 8: 5-amino-N-[3-chloro-4-[4-fluoro-4-(piperazine-1-carbonyl)piperidine-1-carbonyl]phenyl]-1-[2-(difluoromethyl)-3-fluoro-4-(fluoromethoxy)phenyl]imidazole-4-carboxamide;1:1 2,2,2-trifluoroacetic acid

A solution of tert-butyl 4-[1-[4-[[5-amino-1-[2-(difluoromethyl)-3-fluoro-4-(fluoromethoxy)phenyl]imidazole-4-carbonyl]amino]-2-chlorobenzoyl]-4-fluoropiperidine-4-carbonyl]piperazine-1-carboxylate (90.0 mg, 0.12 mmol, 1.0 eq), trifluoroacetic acid (1.0 mL, 12.98 mmol, 111.07 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 30-60) to afford 5-amino-N-[3-chloro-4-[4-fluoro-4-(piperazine-1-carbonyl)piperidine-1-carbonyl]phenyl]-1-[2-(difluoromethyl)-3-fluoro-4-(fluoromethoxy)phenyl]imidazole-4-carboxamide; 1:1 2,2,2-trifluoroacetic acid (28.3 mg, 0.04 mmol, 30.7% yield). MS [M+H]⁺: 670.1

### Compound 212

### (R)-5-amino-N-(3-chloro-4-(4-(morpholine-2-carbonyl)piperazine-1-carbonyl)phenyl)-1-(2-(difluoromethyl)-3-fluoro-4-(fluoromethoxy)phenyl)-1H-imidazole-4-carboxamide;2,2,2-trifluoroacetic acid

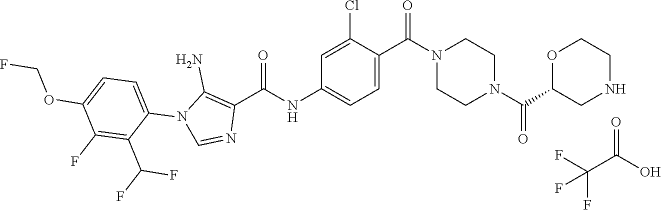

The intermediate 1 was prepared as intermediate

### Step 1: tert-butyl(R)-2-(4-(4-(5-amino-1-(2-(difluoromethyl)-3-fluoro-4-(fluoromethoxy)phenyl)-1H-imidazole-4-carboxamido)-2-chlorobenzoyl)piperazine-1-carbonyl) morpholine-4-carboxylate

2-(7-Azabenzotriazol-1-yl)-N, N,N′,N′-tetramethyluronium hexafluorophosphate (98.79 mg, 0.26 mmol, 1.5 eq) was added to a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide;hydrochloride (100.0 mg, 0.17 mmol, 1.0 eq), (R)-4-(tert-butoxycarbonyl) morpholine-2-carboxylic acid (52.07 mg, 0.23 mmol, 1.3 eq) and triethylamine (0.1 mL, 0.69 mmol, 4.0 eq) in methylene chloride (2 mL) in one portion. The mixture was stirred at 25° C. for 1 h. The reaction was concentrated to dryness. The residue was taken up in EtOAc (30 mL) and the organics washed with 30 mL water followed by 30 mL brine. The organics were then separated and dried (Na₂SO₄) before concentrating to dryness. The crude was then purified by flash column chromatography eluting with of 0˜10% MeOH in methylene chloride to afford tert-butyl(2R)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]morpholine-4-carboxylate (120.0 mg, 0.16 mmol, 79.2% yield). MS [M−boc+H]⁺: 654.3

### Step 2: (R)-5-amino-N-(3-chloro-4-(4-(morpholine-2-carbonyl)piperazine-1-carbonyl)phenyl)-1-(2-(difluoromethyl)-3-fluoro-4-(fluoromethoxy)phenyl)-1H-imidazole-4-carboxamide;2,2,2-trifluoroacetic acid

Trifluoroacetic acid (1.0 mL) was added to a solution of tert-butyl(2R)-2-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]morpholine-4-carboxylate (100.0 mg, 0.13 mmol, 1.0 eq) in methylene chloride (5.0 mL) dropwise. The mixture was stirred at 25° C. for 1 h. The reaction mixture was concentrated to dryness and the residue was purified by prep-HPLC: column Gemini-C18 150×21.2 mm, 5 μm MeCN-H₂O (0.1% TFA) to afford 5-amino-N-[3-chloro-4-[4-[(2R)-morpholine-2-carbonyl]piperazine-1-carbonyl]phenyl]-1-[2-(difluoromethyl)-3-fluoro-4-(fluoromethoxy)phenyl]imidazole-4-carboxamide;2,2,2-trifluoroacetic acid (58.0 mg, 0.08 mmol, 56.5% yield). MS [M+H]⁺: 654.0

### Compound 213

### 5-amino-N-(3-chloro-4-(4-glycylpiperazine-1-carbonyl)phenyl)-1-(2,3-difluoro-4-(fluoromethoxy)phenyl)-1H-imidazole-4-carboxamide; 1:1 2,2,2-trifluoroacetate

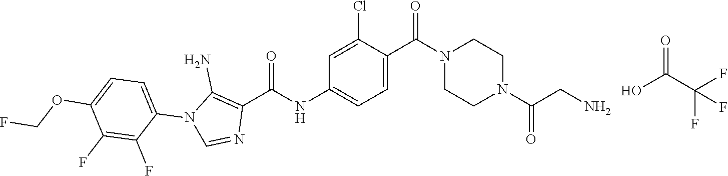 [SMILES: NCC(=O)N1CCN(C(=O)c2ccc(NC(=O)c3ncn(-c4ccc(OCF)c(F)c4F)c3N)cc2Cl)CC1 ; O=C(O)C(F)(F)F]

### Step 1: tert-butyl N-[2-[4-[4-[[5-amino-1-[2,3-difluoro-4-(fluoromethoxy)phenyl]imidazole-4-carbonyl]amino]-2-chlorobenzoyl]piperazin-1-yl]-2-oxo-ethyl]carbamate

A solution of 5-amino-N-[3-chloro-4-(piperazine-1-carbonyl)phenyl]-1-[2,3-difluoro-4-(fluoromethoxy)phenyl]imidazole-4-carboxamide (100.0 mg, 0.2 mmol, 1.0 eq), BOC-glycine (103.27 mg, 0.59 mmol, 3.0 eq), N,N-diisopropylethylamine (0.1 mL, 0.59 mmol, 3.0 eq) and propylphosphonic anhydride solution (374.94 mg, 0.59 mmol, 3.0 eq) in methylene chloride (5 mL) was stirred at 20° C. for 2 h. The resulting solution was concentrated under reduced pressure. The residue was applied on a silica gel column and eluted with 20:1 methylene chloride:MeOH to give tert-butyl N-[2-[4-[4-[[5-amino-1-[2,3-difluoro-4-(fluoromethoxy)phenyl]imidazole-4-carbonyl]amino]-2-chlorobenzoyl]piperazin-1-yl]-2-oxo-ethyl]carbamate (60.0 mg, 0.09 mmol, 34.4% yield). MS [M-Boc+H]⁺: 566.1.

### Step 2: [2-[4-[4-[[5-amino-1-[2,3-difluoro-4-(fluoromethoxy)phenyl]imidazole-4-carbonyl]amino]-2-chlorobenzoyl]piperazin-1-yl]-2-oxo-ethyl]ammonium; 1:1 2,2,2-trifluoroacetate

A solution of tert-butyl N-[2-[4-[4-[[5-amino-1-[2,3-difluoro-4-(fluoromethoxy)phenyl]imidazole-4-carbonyl]amino]-2-chlorobenzoyl]piperazin-1-yl]-2-oxo-ethyl]carbamate (60.0 mg, 0.09 mmol, 1.0 eq) and trifluoroacetic acid (1.0 mL, 12.98 mmol, 144.09 eq) in methylene chloride (5 mL) was stirred at 20° C. for 1 h. The resulting solution was concentrated under reduced pressure. The residue was purified by Prep-HPLC (Gemini-C18 150×19 mm, 5 μm, MeCN-H₂O (0.1% TFA), 35-45) to give [2-[4-[4-[[5-amino-1-[2,3-difluoro-4-(fluoromethoxy)phenyl]imidazole-4-carbonyl]amino]-2-chlorobenzoyl]piperazin-1-yl]-2-oxo-ethyl]ammonium; 1:1 2,2,2-trifluoroacetate (7.4 mg, 0.01 mmol, 12.0% yield). MS [M+H]⁺: 566.0.

### Compound 214

### 4-[4-[[5-amino-1-[2,3-difluoro-4-(fluoromethoxy)phenyl]imidazole-4-carbonyl]amino]-2-chlorobenzoyl]-N-(2-aminoethyl)piperazine-1-carboxamide; 1:1 2,2,2-trifluoroacetic acid

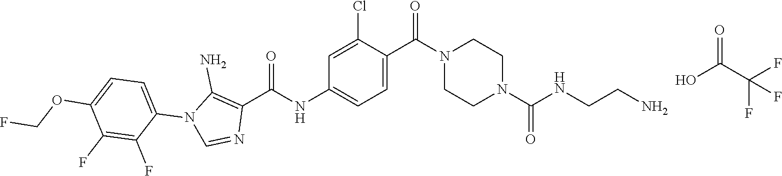 [SMILES: NCCNC(=O)N1CCN(C(=O)c2ccc(NC(=O)c3ncn(-c4ccc(OCF)c(F)c4F)c3N)cc2Cl)CC1 ; O=C(O)C(F)(F)F]

### Step 1: 2,3-difluoro-4-nitrophenol

To a solution of 2,3-difluoro-4-nitroanisole (10.0 g, 52.88 mmol, 1.0 eq) in methylene chloride (30 mL) was added boron tribromide (66.23 g, 264.38 mmol, 5.0 eq) under N₂ atmosphere. The resulting mixture was stirred at 25° C. for 15 h, quenched with MeOH (50 mL) and concentrated under reduced pressure. The residue was purified by flash column chromatography (3:1 petroleum ether:EtOAc) to give 2,3-difluoro-4-nitrophenol (8.3 g, 47.4 mmol, 89.6% yield). MS [M−H]: 174.1.

### Step 2: 2,3-difluoro-1-(fluoromethoxy)-4-nitrobenzene

To a solution of 2,3-difluoro-4-nitrophenol (8300.0 mg, 47.4 mmol, 1.0 eq) in acetonitrile (80 mL) were added fluoro (iodo) methane (11372.03 mg, 71.11 mmol, 1.5 eq) and N,N-diisopropylethylamine (18379.55 mg, 142.21 mmol, 3.0 eq) under N₂ atmosphere. The resulting mixture was stirred at 45° C. for 15 h. After concentration under reduced pressure, the residue was diluted with EtOAc (100 mL), washed with H₂O (3×35 mL), dried over Na₂SO₄ and concentrated under reduced pressure. The residue was purified by flash column chromatography (85:15 petroleum ether:EtOAc) to give 2,3-difluoro-1-(fluoromethoxy)-4-nitrobenzene (9455.0 mg, 45.65 mmol, 96.3% yield).

### Step 3: 2,3-difluoro-4-(fluoromethoxy) aniline

To a solution of 2,3-difluoro-1-(fluoromethoxy)-4-nitrobenzene (9.46 g, 45.6 mmol, 1.0 eq) in ethanol (170 mL) were added iron powder (12.75 g, 228.3 mmol, 5.0 eq), ammonium chloride (24.42 g, 456.52 mmol, 10.0 eq) and water (35 mL) under N₂ atmosphere. The resulting mixture was stirred at 50° C. for 5 h. The EtOH was removed under reduced pressure. The residue was diluted with EtOAc (100 mL), washed with H₂O (3×35 mL), dried over Na₂SO₄ and concentrated under reduced pressure. The residue was purified by flash column chromatography (55:45 petroleum ether:EtOAc) to give 2,3-difluoro-4-(fluoromethoxy) aniline (6000.0 mg, 33.88 mmol, 74.2% yield). MS [M+H]⁺: 178.1.

### Step 4: ethyl 5-amino-1-[2,3-difluoro-4-(fluoromethoxy)phenyl]imidazole-4-carboxylate

To a solution of ethyl 2-amino-2-cyanoacetate, 4-methylbenzenesulfonate (1:1)(13565.04 mg, 45.17 mmol, 2.0 eq) in triethyl orthoformate (100.0 mL, 597.71 mmol, 26.47 eq) was added triethylamine (12.59 mL, 90.33 mmol, 4.0 eq). The resulting mixture was stirred at 82° C. for 2 h, after which time the solvent was removed under reduced pressure. The residue was diluted with MeCN (80 mL), and then 2,3-difluoro-4-(fluoromethoxy) aniline (4000.0 mg, 22.58 mmol, 1.0 eq) was added. The resulting mixture was stirred at 20° C. for 15 h. After concentration under reduced pressure, the residue was purified by flash column chromatography on silica gel (35:65 petroleum ether:EtOAc) to give ethyl 5-amino-1-[2,3-difluoro-4-(fluoromethoxy)phenyl]imidazole-4-carboxylate (5000.0 mg, 15.86 mmol, 70.2% yield). MS [M+H]⁺: 316.1.

### Step 5: 5-amino-1-[2,3-difluoro-4-(fluoromethoxy)phenyl]imidazole-4-carboxylic acid

To a solution of ethyl 5-amino-1-[2,3-difluoro-4-(fluoromethoxy)phenyl]imidazole-4-carboxylate (1500.0 mg, 4.76 mmol, 1.0 eq) in ethanol (45 mL) were added potassium hydroxide (1067.83 mg, 19.03 mmol, 4.0 eq) and water (15.0 mL, 832.63 mmol, 174.99 eq) under N₂. The resulting mixture was stirred at 70° C. for 3 h. The solvent was removed under reduced pressure. The residue was diluted with H₂O (50 mL) and extracted with EtOAc (3×40 mL). The organic layers were discarded, and the aqueous layer was acidified with HCl (4 N) to pH=5 and filtered to give 5-amino-1-[2,3-difluoro-4-(fluoromethoxy)phenyl]imidazole-4-carboxylic acid (1400.0 mg, 4.87 mmol, 71.7% yield), the crude product was used directly without further purification. MS [M+H]⁺: 288.0.

### Step 6: 5-amino-1-[2,3-difluoro-4-(fluoromethoxy)phenyl]imidazole-4-carbonyl chloride

A solution of 5-amino-1-[2,3-difluoro-4-(fluoromethoxy)phenyl]imidazole-4-carboxylic acid (200.0 mg, 0.70 mmol, 1.0 eq) in thionyl chloride (3.0 mL) was stirred at 25° C. for 1 h. The reaction was concentrated to dryness to afford 5-amino-1-[2,3-difluoro-4-(fluoromethoxy)phenyl]imidazole-4-carbonyl chloride (200.0 mg, 0.65 mmol, 74.4% yield). MS [M−Cl+OCH₃+H]⁺: 302.0.

### Step 7: tert-butyl 4-[4-[[5-amino-1-[2,3-difluoro-4-(fluoromethoxy)phenyl]imidazole-4-carbonyl]amino]-2-chlorobenzoyl]piperazine-1-carboxylate

To a solution of tert-butyl 4-(4-amino-2-chlorobenzoyl)piperazine-1-carboxylate (222.37 mg, 0.65 mmol, 1.0 eq) in methylene chloride (3 mL) was added pyridine (258.8 mg, 3.27 mmol, 5.0 eq) and 5-amino-1-[2,3-difluoro-4-(fluoromethoxy)phenyl]imidazole-4-carbonyl chloride (200.0 mg, 0.65 mmol, 1.0 eq). The reaction was stirred at 25° C. for 3 h and then concentrated to dryness. The residue was taken up in EtOAc (20 mL) and washed with 2×20 mL water and 20 mL brine. The organics were then separated and dried (MgSO₄) before concentrating to dryness. The crude material was then purified by flash column chromatography eluting with with 70% EtOAc in petroleum ether to give tert-butyl 4-[4-[[5-amino-1-[2,3-difluoro-4-(fluoromethoxy)phenyl]imidazole-4-carbonyl]amino]-2-chlorobenzoyl]piperazine-1-carboxylate (300.0 mg, 0.49 mmol, 75.3% yield). MS [M+H]⁺: 609.0.

### Step 8: 5-amino-N-[3-chloro-4-(piperazine-1-carbonyl)phenyl]-1-[2,3-difluoro-4-(fluoromethoxy)phenyl]imidazole-4-carboxamide

To a solution of tert-butyl 4-[4-[[5-amino-1-[2,3-difluoro-4-(fluoromethoxy)phenyl]imidazole-4-carbonyl]amino]-2-chlorobenzoyl]piperazine-1-carboxylate (260.0 mg, 0.43 mmol, 1.0 eq) in methylene chloride (3 mL) was added trifluoroacetic acid (1.0 mL). The reaction was stirred at 25° C. for 4 h and then concentrated to dryness to give 5-amino-N-[3-chloro-4-(piperazine-1-carbonyl)phenyl]-1-[2,3-difluoro-4-(fluoromethoxy)phenyl]imidazole-4-carboxamide (173.0 mg, 0.34 mmol, 53.8% yield). MS [M+H]⁺: 509.0.

### Step 9: tert-butyl N-[2-[[4-[4-[[5-amino-1-[2,3-difluoro-4-(fluoromethoxy)phenyl]imidazole-4-carbonyl]amino]-2-chlorobenzoyl]piperazine-1-carbonyl]amino]ethyl]carbamate

To a solution of N-BOC-ethylenediamine (44.08 mg, 0.28 mmol, 2.0 eq) in methylene chloride (3 mL) was added triphosgene (12.25 mg, 0.04 mmol, 0.3 eq). The reaction was stirred at 25° C. for 0.5 h. Next triethylamine (208.79 mg, 2.06 mmol, 15.0 eq) and 5-amino-N-[3-chloro-4-(piperazine-1-carbonyl)phenyl]-1-[2,3-difluoro-4-(fluoromethoxy)phenyl]imidazole-4-carboxamide (70.0 mg, 0.14 mmol, 1.0 eq) were added and the reaction was stirred at 25° C. for 4 h. Concentration to dryness was then followed by purification by flash column chromatography eluting with 80% EtOAc in petroleum ether to afford tert-butyl N-[2-[[4-[4-[[5-amino-1-[2,3-difluoro-4-(fluoromethoxy)phenyl]imidazole-4-carbonyl]amino]-2-chlorobenzoyl]piperazine-1-carbonyl]amino]ethyl]carbamate (30 mg, 0.04 mmol, 30.2% yield). MS [M-Boc+H]⁺: 595.1.

### Step 10: 4-[4-[[5-amino-1-[2,3-difluoro-4-(fluoromethoxy)phenyl]imidazole-4-carbonyl]amino]-2-chlorobenzoyl]-N-(2-aminoethyl)piperazine-1-carboxamide; 1:1 2,2,2-trifluoroacetic acid

A solution of tert-butyl N-[2-[[4-[4-[[5-amino-1-[2,3-difluoro-4-(fluoromethoxy)phenyl]imidazole-4-carbonyl]amino]-2-chlorobenzoyl]piperazine-1-carbonyl]amino]ethyl]carbamate (30.0 mg, 0.04 mmol, 1.0 eq) in methylene chloride (3 mL) was added trifluoroacetic acid (1.0 mL). The reaction was stirred at 25° C. for 3 h. Concentration to dryness followed by purification by Prep-HPLC (Gemini-C18 150×21.2 mm, 5 μm, MeCN— H₂O (0.1% TFA), 35-40) yielded 4-[4-[[5-amino-1-[2,3-difluoro-4-(fluoromethoxy)phenyl]imidazole-4-carbonyl]amino]-2-chlorobenzoyl]-N-(2-aminoethyl)piperazine-1-carboxamide; 1:1 2,2,2-trifluoroacetic acid (7.7 mg, 0.01 mmol, 29.5% yield). MS [M+H]⁺: 595.0.

### Compound 215

### 5-amino-N-[4-[4-(2-aminoethyl)piperidine-1-carbonyl]-3-chlorophenyl]-1-[2,3-difluoro-4-(fluoromethoxy)phenyl]imidazole-4-carboxamide; 1:2 2,2,2-trifluoroacetic acid

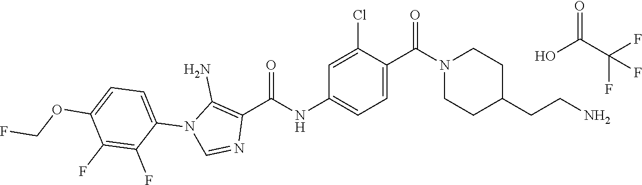

### Step 1: methyl 4-[[5-amino-1-[2,3-difluoro-4-(fluoromethoxy)phenyl]imidazole-4-carbonyl]amino]-2-chlorobenzoate

To a stirring solution of methyl 4-amino-2-chlorobenzoate (182.18 mg, 0.98 mmol, 1.0 eq) and pyridine (0.24 mL, 2.94 mmol, 3.0 eq) in methylene chloride (3 mL) at 0° C. was added 5-amino-1-[2,3-difluoro-4-(fluoromethoxy)phenyl]imidazole-4-carbonyl chloride (300.0 mg, 0.98 mmol, 1.0 eq) in methylene chloride (3 mL). The temperature was increased to 25° C. for 1 h. The resulting solution was concentrated under reduced pressure. The residue was purified on a silica gel column and eluted with 10:1 methylene chloride:MeOH to give methyl 4-[[5-amino-1-[2,3-difluoro-4-(fluoromethoxy)phenyl]imidazole-4-carbonyl]amino]-2-chloro-benzoate (360.0 mg, 0.79 mmol, 64.5% yield). MS [M+H]⁺: 455.0.

### Step 2: 4-[[5-amino-1-[2,3-difluoro-4-(fluoromethoxy)phenyl]imidazole-4-carbonyl]amino]-2-chloro-benzoic acid

To a solution of methyl 4-[[5-amino-1-[2,3-difluoro-4-(fluoromethoxy)phenyl]imidazole-4-carbonyl]amino]-2-chlorobenzoate (360.0 mg, 0.79 mmol, 1.0 eq) in methanol (12 mL) and water (1.5 mL) was added lithium hydroxide (0.04 mL, 3.96 mmol, 5.0 eq). Following 2 h stirring at 50° C. for 2 h, the reaction mixture was diluted with 30 ml of water, adjusted to pH 6˜7 with HCl (2M), and extracted with 3×30 mL of EtOAc. The organic layers were combined and concentrated under reduced pressure to give 4-[[5-amino-1-[2,3-difluoro-4-(fluoromethoxy)phenyl]imidazole-4-carbonyl]amino]-2-chlorobenzoic acid (290.0 mg, 0.66 mmol, 83.1% yield). MS [M+H]⁺: 441.0.

### Step 3: tert-butyl N-[2-[1-[4-[[5-amino-1-[2,3-difluoro-4-(fluoromethoxy)phenyl]imidazole-4-carbonyl]amino]-2-chlorobenzoyl]-4-piperidyl]ethyl]carbamate

To a solution of 4-[[5-amino-1-[2,3-difluoro-4-(fluoromethoxy)phenyl]imidazole-4-carbonyl]amino]-2-chlorobenzoic acid (40.0 mg, 0.09 mmol, 1.0 eq) in DMF (2 mL) were added 4-(2-BOC-aminoethyl)piperidine (20.72 mg, 0.09 mmol, 1.0 eq), 2-(7-azabenzotriazol-1-yl)-N,N,N′,N′-tetramethyluronium hexafluorophosphate (103.52 mg, 0.27 mmol, 3.0 eq) and N,N-diisopropylethylamine (35.19 mg, 0.27 mmol, 3.0 eq). The resulting mixture was stirred at 25° C. for 3 h. The solvent was removed under reduced pressure and the residue was dissolved in EtOAc (30 mL) and washed with H₂O (3×15 mL). The combined organic phases were dried over Na₂SO₄ and concentrated. The crude material was purified by flash column chromatography on silica gel (90:10 methylene chloride:MeOH) to give tert-butyl N-[2-[1-[4-[[5-amino-1-[2,3-difluoro-4-(fluoromethoxy)phenyl]imidazole-4-carbonyl]amino]-2-chlorobenzoyl]-4-piperidyl]ethyl]carbamate (30.0 mg, 0.05 mmol, 49.8% yield). MS [M+H]⁺: 651.2.

### Step 4: 5-amino-N-[4-[4-(2-aminoethyl)piperidine-1-carbonyl]-3-chloro-phenyl]-1-[2,3-difluoro-4-(fluoromethoxy)phenyl]imidazole-4-carboxamide; 1:2 2,2,2-trifluoroacetic acid

To a solution of tert-butyl N-[2-[1-[4-[[5-amino-1-[2,3-difluoro-4-(fluoromethoxy)phenyl]imidazole-4-carbonyl]amino]-2-chlorobenzoyl]-4-piperidyl]ethyl]carbamate (30.0 mg, 0.05 mmol, 1.0 eq) in methylene chloride (1 mL) was added trifluoroacetic acid (1.0 mL, 12.98 mmol, 281.7 eq) under N₂ atmosphere. The resulting mixture was stirred at 20° C. for 1 h. Following concentration under reduced pressure, the residue was purified by prep-HPLC [Gemini-C18, 150×21.2 mm, 5 μm, MeCN-H₂O (0.1% TFA), 25%˜45%] to give 5-amino-N-[4-[4-(2-aminoethyl)piperidine-1-carbonyl]-3-chloro-phenyl]-1-[2,3-difluoro-4-(fluoromethoxy)phenyl]imidazole-4-carboxamide; 1:2 2,2,2-trifluoroacetic acid (11.8 mg, 0.02 mmol, 32.6% yield). MS [M+H]⁺: 551.2.

### Compound 216

### 5-amino-N-[4-[4-(3-aminopropanoyl)piperazine-1-carbonyl]-3-chlorophenyl]-1-[2,3-difluoro-4-(fluoromethoxy)phenyl]imidazole-4-carboxamide; 1:1 2,2,2-trifluoroacetic acid

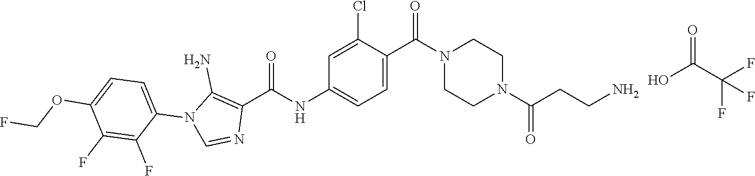 [SMILES: NCCC(=O)N1CCN(C(=O)c2ccc(NC(=O)c3ncn(-c4ccc(OCF)c(F)c4F)c3N)cc2Cl)CC1 ; O=C(O)C(F)(F)F]

### Step 1: tert-butyl N-[3-[4-[4-[[5-amino-1-[2,3-difluoro-4-(fluoromethoxy)phenyl]imidazole-4-carbonyl]amino]-2-chlorobenzoyl]piperazin-1-yl]-3-oxo-propyl]carbamate

A solution of 5-amino-N-[3-chloro-4-(piperazine-1-carbonyl)phenyl]-1-[2,3-difluoro-4-(fluoromethoxy)phenyl]imidazole-4-carboxamide (80.0 mg, 0.16 mmol, 1.0 eq), 5-amino-N-[3-chloro-4-(piperazine-1-carbonyl)phenyl]-1-[2,3-difluoro-4-(fluoromethoxy)phenyl]imidazole-4-carboxamide (80.0 mg, 0.16 mmol, 1.0 eq), BOC-BETA-ALA-OH (32.72 mg, 0.17 mmol, 1.1 eq), N,N-diisopropylethylamine (60.95 mg, 0.47 mmol, 3.0 eq), propyl-phosphonic anhydride (200.08 mg, 0.31 mmol, 2.0 eq) in methylene chloride (4.0 mL) was stirred at 25° C. for 1 h. The resulting solution was concentrated under reduced pressure to give tert-butyl N-[3-[4-[4-[[5-amino-1-[2,3-difluoro-4-(fluoromethoxy)phenyl]imidazole-4-carbonyl]amino]-2-chlorobenzoyl]piperazin-1-yl]-3-oxo-propyl]carbamate (72.0 mg, 0.11 mmol, 57.9% yield). MS [M-Boc+H]⁺: 580.2.

### Step 2: 5-amino-N-[4-[4-(3-aminopropanoyl)piperazine-1-carbonyl]-3-chloro-phenyl]-1-[2,3-difluoro-4-(fluoromethoxy)phenyl]imidazole-4-carboxamide; 1:1 2,2,2-trifluoroacetic acid

A solution of tert-butyl N-[3-[4-[4-[[5-amino-1-[2,3-difluoro-4-(fluoromethoxy)phenyl]imidazole-4-carbonyl]amino]-2-chlorobenzoyl]piperazin-1-yl]-3-oxo-propyl]carbamate (72.0 mg, 0.11 mmol, 1.0 eq), trifluoroacetic acid (1.0 mL, 12.98 mmol, 122.6 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN— H₂O (0.1% TFA), 15-40) to afford 5-amino-N-[4-[4-(3-aminopropanoyl)piperazine-1-carbonyl]-3-chlorophenyl]-1-[2,3-difluoro-4-(fluoromethoxy)phenyl]imidazole-4-carboxamide; 1:1 2,2,2-trifluoroacetic acid (12.2 mg, 0.02 mmol, 16.4% yield). MS [M+H]⁺: 580.1.

### Compound 217

### 5-amino-1-[2-(difluoromethyl)-3-fluoro-4-(fluoromethoxy)phenyl]-N-[3-fluoro-4-[4-(3-fluoroazetidine-3-carbonyl)piperazine-1-carbonyl]phenyl]imidazole-4-carboxamide; 1:1 2,2,2-trifluoroacetic acid

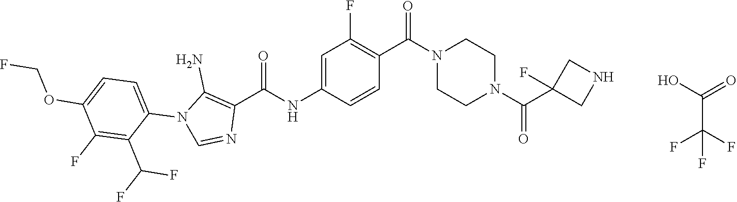 [SMILES: Nc1c(C(=O)Nc2ccc(C(=O)N3CCN(C(=O)C4(F)CNC4)CC3)c(F)c2)ncn1-c1ccc(OCF)c(F)c1C(F)F ; O=C(O)C(F)(F)F]

### Step 1: tert-butyl 3-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-3-fluoroazetidine-1-carboxylate

To a solution of 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (30.0 mg, 0.06 mmol, 1.0 eq) and 1-tert-butoxycarbonyl-3-fluoro-azetidine-3-carboxylic acid (12.54 mg, 0.06 mmol, 1.0 eq) in methylene chloride (1 mL) was added 2-(7-azabenzotriazol-1-yl)-N,N,N′,N′-tetramethyluronium hexafluorophosphate (43.5 mg, 0.11 mmol, 2.0 eq) and triethylamine (0.02 mL, 0.17 mmol, 3.0 eq). The reaction mixture was stirred at 25° C. for 2 h. The reaction was quenched with water (25 mL). The resulting solution was extracted with ethyl acetate (3×20 mL). The combined organic phases were dried over anhydrous sodium sulfate and concentrated under reduced pressure. The residue was purified by flash column (methylene chloride:MeOH=96:4) to give tert-butyl 3-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-3-fluoroazetidine-1-carboxylate (30.0 mg, 0.04 mmol, 69.2% yield). MS [M+H]⁺: 725.2

### Step 2: 5-amino-1-[2-(difluoromethyl)-3-fluoro-4-(fluoromethoxy)phenyl]-N-[3-fluoro-4-[4-(3-fluoroazetidine-3-carbonyl)piperazine-1-carbonyl]phenyl]imidazole-4-carboxamide; 1:1 2,2,2-trifluoroacetic acid

A solution of tert-butyl 3-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-3-fluoroazetidine-1-carboxylate (28.0 mg, 0.04 mmol, 1.0 eq) and trifluoroacetic acid (1.0 mL, 12.98 mmol, 336.39 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA), 25-40) to give 5-amino-1-[2-(difluoromethyl)-3-fluoro-4-(fluoromethoxy)phenyl]-N-[3-fluoro-4-[4-(3-fluoroazetidine-3-carbonyl)piperazine-1-carbonyl]phenyl]imidazole-4-carboxamide; 1:1 2,2,2-trifluoroacetic acid (9.1 mg, 0.01 mmol, 31.5% yield). MS [M+H]⁺: 626.0

### Compound 218

### 5-amino-1-[2,3-difluoro-4-(fluoromethoxy)phenyl]-N-[3-fluoro-4-[4-fluoro-4-(piperazine-1-carbonyl)piperidine-1-carbonyl]phenyl]imidazole-4-carboxamide; 1:1 2,2,2-trifluoroacetic acid

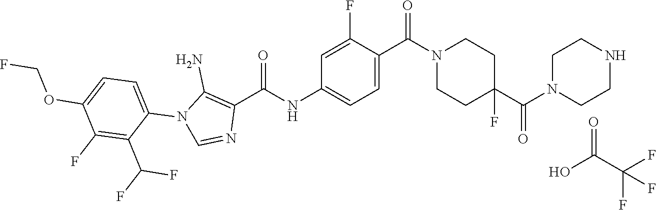

### Step 1: ethyl 4-fluoro-1-(2-fluoro-4-nitrobenzoyl)piperidine-4-carboxylate

To a solution of 2-fluoro-4-nitrobenzoic acid (500.0 mg, 2.7 mmol, 1.0 eq) in methylene chloride (5 mL) were added ethyl 4-fluoropiperidine-4-carboxylate;hydrochloride (628.89 mg, 2.97 mmol, 1.1 eq), 1,3,5,2,4,6-trioxatriphosphorinane (3892.39 mg, 5.4 mmol, 2.0 eq) and N,N-diisopropylethylamine (1047.27 mg, 8.1 mmol, 3.0 eq). The resulting mixture was stirred at 25° C. for 1 h. The solvent was removed under reduced pressure. The residue was diluted with EtOAc (5 mL), washed with H₂O (3×5 mL), dried over Na₂SO₄ and concentrated under reduced pressure to give ethyl 4-fluoro-1-(2-fluoro-4-nitrobenzoyl)piperidine-4-carboxylate (660.0 mg, 1.93 mmol, 71.34% yield). MS [M+H]⁺: 343.0

### Step 2: 4-fluoro-1-(2-fluoro-4-nitrobenzoyl)piperidine-4-carboxylic acid

Ethyl 4-fluoro-1-(2-fluoro-4-nitrobenzoyl)piperidine-4-carboxylate (640.0 mg, 1.87 mmol, 1.0 eq) was dissolved in isopropyl alcohol (10 mL). A solution of sodium hydroxide (373.91 mg, 9.35 mmol, 5.0 eq) in water (5 mL) was added dropwise at 25° C. and stirred at 50° C. for 2 h. The solvent was evaporated. The residue was diluted with water (10 mL) and extracted with ethyl acetate (2×10 mL). The aqueous layer was acidified to pH 5 with HCl (2 M), and the product was collected by filtration to give 4-fluoro-1-(2-fluoro-4-nitrobenzoyl)piperidine-4-carboxylic acid (410.0 mg, 1.3 mmol, 62.8% yield). MS [M+H]⁺: 314.7

### Step 3: tert-butyl 4-[4-fluoro-1-(2-fluoro-4-nitrobenzoyl)piperidine-4-carbonyl]piperazine-1-carboxylate

To a solution of 4-fluoro-1-(2-fluoro-4-nitrobenzoyl)piperidine-4-carboxylic acid (200.0 mg, 0.64 mmol, 1.0 eq) in methylene chloride (5 mL) were added 1-BOC-piperazine (130.39 mg, 0.7 mmol, 1.1 eq), 2-(7-azabenzotriazol-1-yl)-N,N,N′,N′-tetramethyluronium hexafluorophosphate (362.78 mg, 1.0 mmol, 1.5 eq) and N,N-diisopropylethylamine (246.77 mg, 1.91 mmol, 3.0 eq). The resulting mixture was stirred at 25° C. for 1 h. The solvent was removed under reduced pressure. The residue was diluted with EtOAc (5 mL) and washed with H₂O (3×5 mL), dried over Na₂SO₄ and concentrated under reduced pressure to give tert-butyl 4-[4-fluoro-1-(2-fluoro-4-nitrobenzoyl)piperidine-4-carbonyl]piperazine-1-carboxylate (197.0 mg, 0.41 mmol, 64.2% yield). MS [M+Na]⁺: 505.1

### Step 4: tert-butyl 4-[1-(4-amino-2-fluorobenzoyl)-4-fluoropiperidine-4-carbonyl]piperazine-1-carboxylate

A solution of tert-butyl 4-[4-fluoro-1-(2-fluoro-4-nitrobenzoyl)piperidine-4-carbonyl]piperazine-1-carboxylate (177.0 mg, 0.37 mmol, 1.0 eq), ammonium chloride (98.12 mg, 1.83 mmol, 5.0 eq) and iron powder (102.35 mg, 1.83 mmol, 5.0 eq) in ethanol (0.961 mL) was stirred at 50° C. for 2 h. The resulting solution was extracted with by water and ethyl acetate followed by concentration under reduced pressure at 30° C. The residue was purified by flash chromatography (silica gel, 0 to 10% MeOH in methylene chloride) to tert-butyl 4-[1-(4-amino-2-fluorobenzoyl)-4-fluoropiperidine-4-carbonyl]piperazine-1-carboxylate (126.0 mg, 0.28 mmol, 75.9% yield). MS [M+H]⁺: 453.3

### Step 5: tert-butyl 4-[1-[4-[[5-amino-1-[2,3-difluoro-4-(fluoromethoxy)phenyl]imidazole-4-carbonyl]amino]-2-fluorobenzoyl]-4-fluoropiperidine-4-carbonyl]piperazine-1-carboxylate

To a solution of tert-butyl 4-[1-(4-amino-2-fluorobenzoyl)-4-fluoropiperidine-4-carbonyl]piperazine-1-carboxylate (41.45 mg, 0.09 mmol, 0.7 eq) and pyridine (0.21 mL, 2.62 mmol, 20.0 eq) in methylene chloride (1 mL) was added a suspension of 5-amino-1-[2,3-difluoro-4-(fluoromethoxy)phenyl]imidazole-4-carbonyl chloride (40.0 mg, 0.13 mmol, 1.0 eq) in methylene chloride (1 mL) at 25° C. The mixture was stirred at 25° C. for 1 h under N₂. The resulting solution was evaporated under reduced pressure. The residue was purified by column chromatography (silica gel, 0 to 10% MeOH in methylene chloride) to give tert-butyl 4-[1-[4-[[5-amino-1-[2,3-difluoro-4-(fluoromethoxy)phenyl]imidazole-4-carbonyl]amino]-2-fluorobenzoyl]-4-fluoropiperidine-4-carbonyl]piperazine-1-carboxylate (40.0 mg, 0.06 mmol, 42.4% yield). MS [M+H]⁺: 722.3

### Step 6: 5-amino-1-[2,3-difluoro-4-(fluoromethoxy)phenyl]-N-[3-fluoro-4-[4-fluoro-4-(piperazine-1-carbonyl)piperidine-1-carbonyl]phenyl]imidazole-4-carboxamide; 1:1 2,2,2-trifluoroacetic acid

A solution of tert-butyl 4-[1-[4-[[5-amino-1-[2,3-difluoro-4-(fluoromethoxy)phenyl]imidazole-4-carbonyl]amino]-2-fluorobenzoyl]-4-fluoropiperidine-4-carbonyl]piperazine-1-carboxylate (30.0 mg, 0.04 mmol, 1.0 eq) and trifluoroacetic acid (4.74 mg, 0.04 mmol, 1.0 eq) in methylene chloride (1 mL) was stirred at 25° C. for 1 h. The resulting solution was concentrated under reduced pressure. The crude product was purified by Prep-HPLC (Gemini-C18 150×21.2 mm, 5 μm, MeCN-H₂O (0.1% TFA), 15-45) to give 5-amino-1-[2,3-difluoro-4-(fluoromethoxy)phenyl]-N-[3-fluoro-4-[4-fluoro-4-(piperazine-1-carbonyl)piperidine-1-carbonyl]phenyl]imidazole-4-carboxamide; 1:1 2,2,2-trifluoroacetic acid (14.9 mg, 0.02 mmol, 48.4% yield). MS [M+H]⁺: 622.3

### Compound 219

### 5-amino-N-[4-[4-[(1S,5S)-3-azabicyclo[3.1.0]hexane-1-carbonyl]piperazine-1-carbonyl]-3-chloro-phenyl]-1-[2-(difluoromethyl)-3-fluoro-4-(fluoromethoxy)phenyl]imidazole-4-carboxamide; 1:1 2,2,2-trifluoroacetic acid

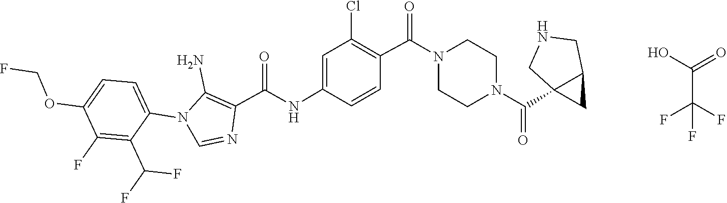 [SMILES: Nc1c(C(=O)Nc2ccc(C(=O)N3CCN(C(=O)[C@]45CNC[C@H]4C5)CC3)c(Cl)c2)ncn1-c1ccc(OCF)c(F)c1C(F)F ; O=C(O)C(F)(F)F]

### Step 1: tert-butyl(1S,5S)-1-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate

To a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide (50.0 mg, 0.09 mmol, 1.0 eq) and (1S,5S)-3-tert-butoxycarbonyl-3-azabicyclo[3.1.0]hexane-1-carboxylic acid (21.01 mg, 0.09 mmol, 1.0 eq) in methylene chloride (1 mL) was added 2-(7-azabenzotriazol-1-yl)-N,N,N′,N′-tetramethyluronium hexafluorophosphate (70.3 mg, 0.18 mmol, 2.0 eq) and triethylamine (0.04 mL, 0.28 mmol, 3.0 eq). The mixture was stirred at 25° C. for 2 h. The reaction was quenched with water (25 mL). The resulting solution was extracted with ethyl acetate (3×20 mL). The combined organic phases were dried over anhydrous sodium sulfate and concentrated under reduced pressure. The residue was purified by flash column to give tert-butyl(1S,5S)-1-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (45.0 mg, 0.06 mmol, 64.1% yield). MS [M+H]⁺: 750.2

### Step 2: 5-amino-N-[4-[4-[(1S,5S)-3-azabicyclo[3.1.0]hexane-1-carbonyl]piperazine-1-carbonyl]-3-chloro-phenyl]-1-[2-(difluoromethyl)-3-fluoro-4-(fluoromethoxy)phenyl]imidazole-4-carboxamide; 1:1 2,2,2-trifluoroacetic acid

A solution of tert-butyl(1S,5S)-1-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (50.0 mg, 0.07 mmol, 1.0 eq) and trifluoroacetic acid (1.0 mL, 12.98 mmol, 194.74 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN— H₂O (0.1% TFA), 25-40) to give 5-amino-N-[4-[4-[(1S,5S)-3-azabicyclo[3.1.0]hexane-1-carbonyl]piperazine-1-carbonyl]-3-chloro-phenyl]-1-[2-(difluoromethyl)-3-fluoro-4-(fluoromethoxy)phenyl]imidazole-4-carboxamide; 1:1 2,2,2-trifluoroacetic acid (23.6 mg, 0.03 mmol, 45.9% yield). MS [M+H]⁺: 650.2

### Compound 220

### N-(4-(4-((1R,5S,6s)-3-azabicyclo[3.1.0]hexane-6-carbonyl)piperazine-1-carbonyl)-3-chlorophenyl)-5-amino-1-(2-(difluoromethyl)-3-fluoro-4-(fluoromethoxy)phenyl)-1H-imidazole-4-carboxamide;2,2,2-trifluoroacetic acid

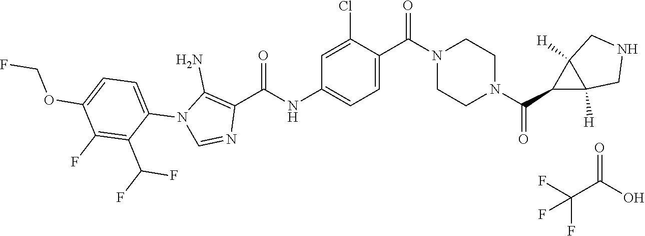

The intermediate 1 was prepared as intermediate

### Step 1: tert-butyl(1R,5S,6s)-6-(4-(4-(5-amino-1-(2-(difluoromethyl)-3-fluoro-4-(fluoromethoxy)phenyl)-1H-imidazole-4-carboxamido)-2-chlorobenzoyl)piperazine-1-carbonyl)-3-azabicyclo[3.1.0]hexane-3-carboxylate

O-(7-Azabenzotriazol-1-yl)-N,N,N′,N′-tetramethyluronium hexafluorophosphate (52.72 mg, 0.14 mmol, 1.5 eq) was added to a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide (50.0 mg, 0.09 mmol, 1.0 eq), (1S,5R)-3-tert-butoxycarbonyl-3-azabicyclo[3.1.0]hexane-6-carboxylic acid (27.31 mg, 0.12 mmol, 1.3 eq) and triethylamine (0.05 mL, 0.37 mmol, 4.0 eq) in methylene chloride (2 mL), the mixture was stirred at 25° C. for 2 h. The reaction was concentrated to dryness and the residue was taken up in EtOAc (20 mL) and the organics washed with 20 ml water and 20 mL brine. The organics were then separated and dried (Na₂SO₄) before concentrating to dryness. The crude was then purified by flash column chromatography eluting with 0˜10% MeOH in methylene chloride to afford tert-butyl(1S,5R)-6-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (50.0 mg, 0.07 mmol, 71.0% yield). MS [M+H−Boc]⁺: 650.3

### Step 2: N-(4-(4-((1R,5S,6s)-3-azabicyclo[3.1.0]hexane-6-carbonyl)piperazine-1-carbonyl)-3-chlorophenyl)-5-amino-1-(2-(difluoromethyl)-3-fluoro-4-(fluoromethoxy)phenyl)-1H-imidazole-4-carboxamide;2,2,2-trifluoroacetic acid

Trifluoroacetic acid (0.5 mL) was added to a solution of tert-butyl(1S,5R)-6-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (40.0 mg, 0.05 mmol, 1.0 eq) in methylene chloride (2 mL). The mixture was stirred at 25° C. for 1 h. The mixture was concentrated then purified by prep-HPLC: column Gemini-C18 150×21.2 mm, um MeCN-H₂O (0.1% TFA) to afford 5-amino-N-[4-[4-[(1S,5R)-3-azabicyclo[3.1.0]hexane-6-carbonyl]piperazine-1-carbonyl]-3-chloro-phenyl]-1-[2-(difluoromethyl)-3-fluoro-4-(fluoromethoxy)phenyl]imidazole-4-carboxamide;2,2,2-trifluoroacetic acid (29.7 mg, 0.04 mmol, 72.9% yield). MS [M+H]⁺: 650.3

### Compound 221

### 5-amino-N-[4-[4-[(1R,5R)-3-azabicyclo[3.1.0]hexane-1-carbonyl]piperazine-1-carbonyl]-3-chloro-phenyl]-1-[2,3-difluoro-4-(fluoromethoxy)phenyl]imidazole-4-carboxamide; 1:1 2,2,2-trifluoroacetic acid

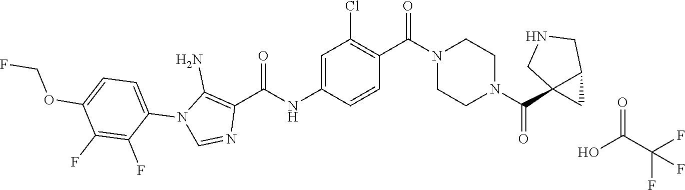

### Step 1: tert-butyl(1R,5R)-1-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate

To a solution of 5-amino-N-[3-chloro-4-(piperazine-1-carbonyl)phenyl]-1-[2,3-difluoro-4-(fluoromethoxy)phenyl]imidazole-4-carboxamide (100.0 mg, 0.2 mmol, 1.0 eq) in methylene chloride (3 mL) were added (1R,5R)-3-tert-butoxycarbonyl-3-azabicyclo[3.1.0]hexane-1-carboxylic acid (66.99 mg, 0.29 mmol, 1.5 eq), 2-(7-aza-1H-benzotriazole-1-yl)-1,1,3,3-tetramethyluronium hexafluorophosphate (149.44 mg, 0.39 mmol, 2.0 eq) and N,N-diisopropylethylamine (0.1 mL, 0.59 mmol, 3.0 eq). The resulting mixture was stirred at 25° C. for 1 h. The solvent was removed under reduced pressure. The residue was diluted with EtOAc (5 mL), washed with H₂O (3×5 mL), dried over Na₂SO₄ and concentrated under reduced pressure to give tert-butyl (1R,5R)-1-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (85.0 mg, 0.12 mmol, 50.0% yield). MS [M-Boc+H]⁺: 618.1

### Step 2: 5-amino-N-[4-[4-[(1R,5R)-3-azabicyclo[3.1.0]hexane-1-carbonyl]piperazine-1-carbonyl]-3-chloro-phenyl]-1-[2,3-difluoro-4-(fluoromethoxy)phenyl]imidazole-4-carboxamide; 1:1 2,2,2-trifluoroacetic acid

A solution of tert-butyl(1R,5R)-1-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (85.0 mg, 0.12 mmol, 1.0 eq), trifluoroacetic acid (1.0 mL, 13 mmol, 110 eq) in methylene chloride (4 mL) was stirred at 25° C. for 1 h. The resulting solution was concentrated under reduced pressure. The crude product was purified by Prep-HPLC (Gemini C18 150×21.2 mm, 5 μm, MeCN-H₂O (0.1% TFA), 30-60) to afford 5-amino-N-[4-[4-[(1R,5R)-3-azabicyclo[3.1.0]hexane-1-carbonyl]piperazine-1-carbonyl]-3-chloro-phenyl]-1-[2,3-difluoro-4-(fluoromethoxy)phenyl]imidazole-4-carboxamide; 1:1 2,2,2-trifluoroacetic acid (65.8 mg, 0.09 mmol, 75.8% yield). MS [M+H]⁺: 618.1

### Compound 222

### 5-amino-N-[4-[4-[(1S,5S)-3-azabicyclo[3.1.0]hexane-1-carbonyl]piperazine-1-carbonyl]-3-chloro-phenyl]-1-[2,3-difluoro-4-(fluoromethoxy)phenyl]imidazole-4-carboxamide; 1:1 2,2,2-trifluoroacetic acid

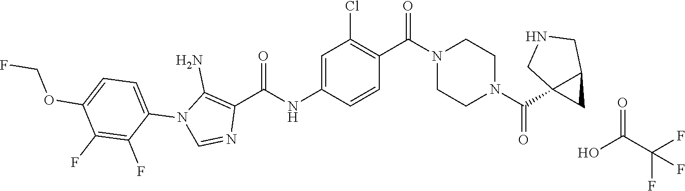

### Step 1: tert-butyl(1S,5S)-1-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate

To a solution of 5-amino-N-[3-chloro-4-(piperazine-1-carbonyl)phenyl]-1-[2,3-difluoro-4-(fluoromethoxy)phenyl]imidazole-4-carboxamide (90.0 mg, 0.18 mmol, 1.0 eq) in methylene chloride (5 mL) were added (1S,5S)-3-tert-butoxycarbonyl-3-azabicyclo[3.1.0]hexane-1-carboxylic acid (52.25 mg, 0.23 mmol, 1.3 eq),N,N-diisopropylethylamine (57.14 mg, 0.44 mmol, 2.5 eq) and O-(7-azabenzotriazol-1-yl)-N,N,N′,N′-tetramethyluronium hexafluorophosphate (121.04 mg, 0.32 mmol, 1.8 eq) under N₂ atmosphere. The resulting mixture was stirred at 25° C. for 1 h. It was then diluted with EtOAc (30 mL), washed with H₂O (3×25 mL), dried over Na₂SO₄ and concentrated under reduced pressure to give tert-butyl(1S,5S)-1-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (104.0 mg, 0.14 mmol, 68.0% yield). The crude product was used directly without further purification. MS [M+H]⁺: 718.2

### Step 2: 5-amino-N-[4-[4-[(1S,5S)-3-azabicyclo[3.1.0]hexane-1-carbonyl]piperazine-1-carbonyl]-3-chloro-phenyl]-1-[2,3-difluoro-4-(fluoromethoxy)phenyl]imidazole-4-carboxamide; 1:1 2,2,2-trifluoroacetic acid

To a solution of tert-butyl(1S,5S)-1-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (104.0 mg, 0.12 mmol, 1.0 eq) in methylene chloride (2 mL) was added trifluoroacetic acid (0.83 mL, 10.84 mmol, 90.17 eq). The resulting mixture was stirred at 25° C. for 1 h. After concentration under reduced pressure, the residue was purified by prep-HPLC [Gemini-C18 150×21.2 mm, 5 μm, MeCN-H₂O (0.1% TFA), 40%˜60%] to give 5-amino-N-[4-[4-[(1S,5S)-3-azabicyclo[3.1.0]hexane-1-carbonyl]piperazine-1-carbonyl]-3-chloro-phenyl]-1-[2,3-difluoro-4-(fluoromethoxy)phenyl]imidazole-4-carboxamide; 1:1 2,2,2-trifluoroacetic acid (57.3 mg, 0.08 mmol, 64.5% yield). MS [M+H]⁺: 618.1

### Compound 223

### 5-amino-N-[4-[4-[(1R,5R)-3-azabicyclo[3.1.0]hexane-1-carbonyl]piperazine-1-carbonyl]-3-chloro-phenyl]-1-[2-(difluoromethyl)-3-fluoro-4-(fluoromethoxy)phenyl]imidazole-4-carboxamide; 1:1 2,2,2-trifluoroacetic acid

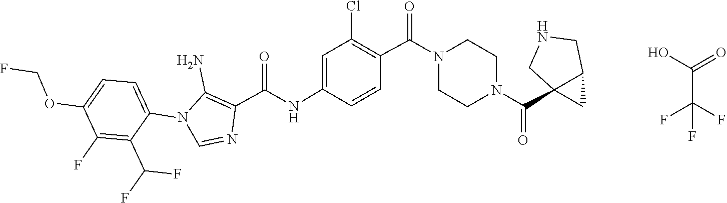 [SMILES: Nc1c(C(=O)Nc2ccc(C(=O)N3CCN(C(=O)[C@@]45CNC[C@@H]4C5)CC3)c(Cl)c2)ncn1-c1ccc(OCF)c(F)c1C(F)F ; O=C(O)C(F)(F)F]

### Step 1: tert-butyl(1R,5R)-1-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate

To a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide (50.0 mg, 0.09 mmol, 1.0 eq) and (1R,5R)-3-tert-butoxycarbonyl-3-azabicyclo[3.1.0]hexane-1-carboxylic acid (21.01 mg, 0.09 mmol, 1.0 eq) in methylene chloride (1 mL) was added 2-(7-azabenzotriazol-1-yl)-N,N,N′,N′-tetramethyluronium hexafluorophosphate (70.3 mg, 0.18 mmol, 2.0 eq) and triethylamine (0.04 mL, 0.28 mmol, 3.0 eq), the mixture was stirred at 25° C. for 2 h. The reaction was quenched with water (25 mL). The resulting solution was extracted with ethyl acetate (3×20 mL). The combined organic phases were dried over anhydrous Na₂SO₄ and concentrated under reduced pressure. The residue was purified by flash column to give tert-butyl(1R,5R)-1-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (45.0 mg, 0.06 mmol, 64.9% yield). MS [M+H]⁺: 750.2

### Step 2: 5-amino-N-[4-[4-[(1R,5R)-3-azabicyclo[3.1.0]hexane-1-carbonyl]piperazine-1-carbonyl]-3-chloro-phenyl]-1-[2-(difluoromethyl)-3-fluoro-4-(fluoromethoxy)phenyl]imidazole-4-carboxamide; 1:1 2,2,2-trifluoroacetic acid

A solution of tert-butyl(1R,5R)-1-[4-[4-[[5-amino-1-[2-(difluoromethyl)-3-fluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (50.0 mg, 0.07 mmol, 1.0 eq) and trifluoroacetic acid (1.0 mL, 12.98 mmol, 194.74 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA), 25-40) to give 5-amino-N-[4-[4-[(1R,5R)-3-azabicyclo[3.1.0]hexane-1-carbonyl]piperazine-1-carbonyl]-3-chloro-phenyl]-1-[2-(difluoromethyl)-3-fluoro-4-(fluoromethoxy)phenyl]imidazole-4-carboxamide; 1:1 2,2,2-trifluoroacetic acid (24.6 mg, 0.03 mmol, 56.3% yield). MS [M+H]⁺: 650.0

### Compound 224

### 5-amino-N-[4-[4-[(1R,5S,6r)-3-azabicyclo[3.1.0]hexane-6-carbonyl]piperazine-1-carbonyl]-3-chloro-phenyl]-1-[2,3-difluoro-4-(fluoromethoxy)phenyl]imidazole-4-carboxamide; 1:1 2,2,2-trifluoroacetic acid

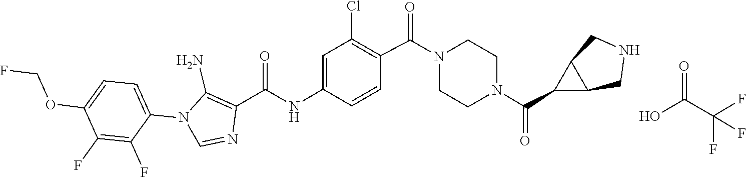 [SMILES: Nc1c(C(=O)Nc2ccc(C(=O)N3CCN(C(=O)[C@@H]4[C@@H]5CNC[C@@H]54)CC3)c(Cl)c2)ncn1-c1ccc(OCF)c(F)c1F ; O=C(O)C(F)(F)F]

### Step 1: tert-butyl 4-[4-[[5-amino-1-[2,3-difluoro-4-(fluoromethoxy)phenyl]imidazole-4-carbonyl]amino]-2-chlorobenzoyl]piperazine-1-carboxylate

To a solution of tert-butyl 4-(4-amino-2-chlorobenzoyl)piperazine-1-carboxylate (999.7 mg, 2.94 mmol, 0.65 eq) in methylene chloride (15 mL) was added pyridine (1790.0 mg, 22.63 mmol, 5.0 eq) and 5-amino-1-[2,3-difluoro-4-(fluoromethoxy)phenyl]imidazole-4-carbonyl chloride (1590.0 mg, 4.53 mmol, 1.0 eq) under N₂ atmosphere. The resulting mixture was stirred at 25° C. for 1 h. Diluted with methylene chloride (50 mL) and washed with citric acid aqueous solution (3×20 mL), dried over Na₂SO₄ and concentrated under reduced pressure and the residue was purified by flash column chromatography (3:2 petroleum ether:EtOAc) to give tert-butyl 4-[4-[[5-amino-1-[2,3-difluoro-4-(fluoromethoxy)phenyl]imidazole-4-carbonyl]amino]-2-chlorobenzoyl]piperazine-1-carboxylate (2400.0 mg, 3.94 mmol, 74.9% yield). MS [M+H]⁺: 608.9

### Step 2: 5-amino-N-[3-chloro-4-(piperazine-1-carbonyl)phenyl]-1-[2,3-difluoro-4-(fluoromethoxy)phenyl]imidazole-4-carboxamide; 1:1 hydrochloride

To a solution of tert-butyl 4-[4-[[5-amino-1-[2,3-difluoro-4-(fluoromethoxy)phenyl]imidazole-4-carbonyl]amino]-2-chlorobenzoyl]piperazine-1-carboxylate (2400.0 mg, 3.39 mmol, 1.0 eq) in methylene chloride (10 mL) was added hydrochloric acid (4M in dioxane)(5.0 mL, 20.0 mmol, 5.9 eq). The resulting mixture was stirred at 25° C. for 3 h. Concentration under reduced pressure afforded 5-amino-N-[3-chloro-4-(piperazine-1-carbonyl)phenyl]-1-[2,3-difluoro-4-(fluoromethoxy)phenyl]imidazole-4-carboxamide; 1:1 hydrochloride (1820.0 mg, 3.34 mmol, 82.7% yield), the crude product was used directly without further purification. MS [M+H]⁺: 508.9

### Step 3: tert-butyl(1R,5S)-6-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate

To a solution of 5-amino-N-[3-chloro-4-(piperazine-1-carbonyl)phenyl]-1-[2,3-difluoro-4-(fluoromethoxy)phenyl]imidazole-4-carboxamide (80.0 mg, 0.16 mmol, 1.0 eq) in methylene chloride (5 mL) were added (1R,5S)-3-tert-butoxycarbonyl-3-azabicyclo[3.1.0]hexane-6-carboxylic acid (46.44 mg, 0.2 mmol, 1.3 eq), N,N-diisopropylethylamine (50.79 mg, 0.39 mmol, 2.5 eq) and O-(7-azabenzotriazol-1-yl)-N,N,N′,N′-tetramethyluronium hexafluorophosphate (107.59 mg, 0.28 mmol, 1.8 eq) under N₂ atmosphere. The resulting mixture was stirred at 25° C. for 1 h. Diluted with EtOAc (30 mL) and washed with H₂O (3×25 mL), dried over Na₂SO₄ and concentrated under reduced pressure to give tert-butyl(1R,5S)-6-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (91.0 mg, 0.13 mmol, 62.1% yield). MS [M-Boc+H]⁺: 618.3

### Step 4: 5-amino-N-[4-[4-[(1R,5S)-3-azabicyclo[3.1.0]hexane-6-carbonyl]piperazine-1-carbonyl]-3-chloro-phenyl]-1-[2,3-difluoro-4-(fluoromethoxy)phenyl]imidazole-4-carboxamide; 1:1 2,2,2-trifluoroacetic acid

To a solution of tert-butyl(1R,5S)-6-[4-[4-[[5-amino-1-[2,3-difluoro-4-(fluoromethoxy)phenyl]imidazole-4-carbonyl]amino]-2-chlorobenzoyl]piperazine-1-carbonyl]-3-azabicyclo[3.1.0]hexane-3-carboxylate (91.0 mg, 0.1 mmol, 1.0 eq) in methylene chloride (2 mL) was added trifluoroacetic acid (1.0 mL, 13.06 mmol, 133.84 eq). The resulting mixture was stirred at 25° C. for 1 h. After concentration under reduced pressure, the residue was purified by prep-HPLC [Gemini-C18 150×21.2 mm, 5 μm, MeCN-H₂O (0.1% TFA), 40%˜55%] to give 5-amino-N-[4-[4-[(1R,5S)-3-azabicyclo[3.1.0]hexane-6-carbonyl]piperazine-1-carbonyl]-3-chloro-phenyl]-1-[2,3-difluoro-4-(fluoromethoxy)phenyl]imidazole-4-carboxamide;2,2,2-trifluoroacetic acid (48.4 mg, 0.07 mmol, 67.1% yield). MS [M+H]⁺: 618.0

### Compound 225

### 5-amino-N-[3-chloro-4-[4-(piperazine-1-carbonyl)piperazine-1-carbonyl]phenyl]-1-[4-(cyclopropoxy)-2,3-difluoro-phenyl]imidazole-4-carboxamide; 1:1 2,2,2-trifluoroacetic acid

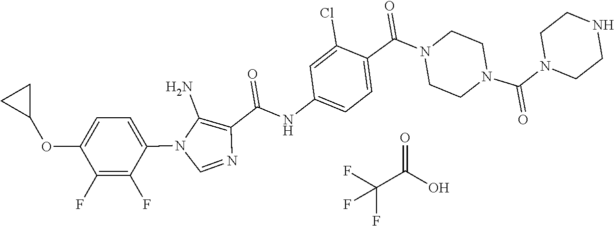 [SMILES: Nc1c(C(=O)Nc2ccc(C(=O)N3CCN(C(=O)N4CCNCC4)CC3)c(Cl)c2)ncn1-c1ccc(OC2CC2)c(F)c1F ; O=C(O)C(F)(F)F]

### Step 1: 3-chloro-1-(cyclopropoxy)-2-fluoro-4-nitrobenzene

To a solution of cyclopropanol (0.48 g, 8.27 mmol, 0.64 eq) in THF (35 mL) was added sodium hydride (0.31 g, 12.92 mmol, 1.0 eq) at 0° C. under nitrogen atmosphere. The mixture was stirred at 0° C. for 0.5 hr. Then 3-chloro-1,2-difluoro-4-nitrobenzene (2.5 g, 12.92 mmol, 1.0 eq) was added. The mixture was stirred at 25° C. for 16 h. The reaction mixture was quenched by addition of saturated ammonium chloride solution 40 mL at 0° C., and then extracted with ethyl acetate (3×20 mL). The combined organic layers were washed with brine 10 mL, dried over anhydrous sodium sulfate, filtered, and concentrated under reduced pressure to afford residue. The residue was purified by column chromatography eluting with 10% EtOAc in isohexane to afford 3-chloro-1-(cyclopropoxy)-2-fluoro-4-nitrobenzene (2100.0 mg, 9.07 mmol, 70.2% yield).

¹H NMR (400 MHz, DMSO) δ 8.11 (dd, J=9.3, 1.9 Hz, 1H), 7.62 (dd, J=9.2, 8.2 Hz, 1H), 4.18 (dt, J=8.9, 3.0 Hz, 1H), 0.88 (dd, J=10.8, 4.8 Hz, 2H), 0.83-0.79 (m, 2H).

### Step 2: 1-(cyclopropoxy)-2,3-difluoro-4-nitrobenzene

To a solution of 3-chloro-1-(cyclopropoxy)-2-fluoro-4-nitrobenzene (2100.0 mg, 9.07 mmol, 1.0 eq) in DMSO (40 mL) was added cesium fluoride (0.67 mL, 18.1 mmol, 2.0 eq). The mixture was stirred at 140° C. for 5 h. The reaction mixture was quenched by addition water 30 mL at 0° C., and then extracted with ethyl acetate (3×40 mL). The combined organic layers were washed with brine 40 mL, dried over anhydrous sodium sulfate, filtered, and concentrated under reduced pressure to afford residue. The residue was purified by flash column chromatography eluting with 10% EtOAc in isohexane to afford 1-(cyclopropoxy)-2,3-difluoro-4-nitrobenzene (1200.0 mg, 5.58 mmol, 49.2% yield).

### Step 3: 4-(cyclopropoxy)-2,3-difluoro-aniline

To a solution of 1-(cyclopropoxy)-2,3-difluoro-4-nitrobenzene (1200.0 mg, 5.58 mmol, 1.0 eq) in ethanol (12 mL) and water (3 mL) was added iron powder (1561.63 mg, 27.96 mmol, 5.0 eq) and ammonium chloride (1491.63 mg, 27.89 mmol, 5.0 eq). The reaction was stirred at 50° C. for 5 h, the solid was filtered off, and the filtrate was diluted with EtOAc (50 mL) and washed with water (3×25 mL), dried over Na₂SO₄ and evaporated under reduced pressure to afford product 4-(cyclopropoxy)-2,3-difluoroaniline (720.0 mg, 3.89 mmol, 64.3% yield). MS [M+H]⁺: 186.1

### Step 4: ethyl 5-amino-1-[4-(cyclopropoxy)-2,3-difluorophenyl]imidazole-4-carboxylate

To a solution of ethyl 2-amino-2-cyanoacetate; 4-methylbenzene-1-sulfonic acid (973.15 mg, 3.24 mmol, 2.0 eq) were added triethylamine (0.9 mL, 6.48 mmol, 4.0 eq) and triethyl orthoformate (3503.11 mg, 23.64 mmol, 14.59 eq). The resulting mixture was stirred at 80° C. for 1.5 h. The solvent was removed under reduced pressure and the residue was diluted with MeCN (4 mL). Next 4-(cyclopropoxy)-2,3-difluoro-aniline (300.0 mg, 1.62 mmol, 1.0 eq) was added and the resulting mixture was stirred at 50° C. for 16 h. The reaction was concentrated to dryness and the residue was taken up in EtOAc (30 mL) and the organics washed with 2×30 mL water followed by 30 mL brine. The organics were then separated and dried (MgSO₄) before concentrating to dryness. The crude was then purified by flash column chromatography eluting with 80% EtOAc in petroleum ether to afford ethyl 5-amino-1-[4-(cyclopropoxy)-2,3-difluoro-phenyl]imidazole-4-carboxylate (270.0 mg, 0.84 mmol, 51.6% yield). MS [M+H]⁺: 324.0

### Step 5: 5-amino-1-[4-(cyclopropoxy)-2,3-difluoro-phenyl]imidazole-4-carboxylic acid

Sodium hydroxide (105.16 mg, 2.63 mmol, 5.0 eq) was added to the solution of 5-amino-1-[4-(cyclopropoxy)-2,3-difluoro-phenyl]imidazole-4-carboxylate (170.0 mg, 0.53 mmol, 1.0 eq) in isopropyl alcohol (20 mL) and water (10 mL). The solution was stirred at 50° C. for 12 h. The solvent was then evaporated. The residue was diluted with water (10 mL), and the solution was extracted with ethyl acetate (2×10 mL). The aqueous layer was acidified to pH=5 with HCl (2 M), and the product was collected by filtration to afford 5-amino-1-[4-(cyclopropoxy)-2,3-difluorophenyl]imidazole-4-carboxylic acid (100.0 mg, 0.34 mmol, 64.4% yield). MS [M+H]⁺: 296.0

### Step 6: 5-amino-1-[4-(cyclopropoxy)-2,3-difluorophenyl]imidazole-4-carbonyl chloride

5-amino-1-[4-(cyclopropoxy)-2,3-difluorophenyl]imidazole-4-carboxylic acid (100.0 mg, 0.34 mmol, 1.0 eq) was dissolved in thionyl chloride (0.5 mL) at 25° C. under N₂. The solution was stirred at 25° C. for 1 h under N₂. The resulting solution was evaporated to dryness and azeotroped with methylene chloride to afford the title compound 5-amino-1-[4-(cyclopropoxy)-2,3-difluorophenyl]imidazole-4-carbonyl chloride (100.0 mg, 0.32 mmol, 65.2% yield), which was used without further purification. MS [M−Cl+OCH₃+H]⁺: 310.1

### Step 7: tert-butyl 4-[4-[4-[[5-amino-1-[4-(cyclopropoxy)-2,3-difluoro-phenyl]imidazole-4-carbonyl]amino]-2-chlorobenzoyl]piperazine-1-carbonyl]piperazine-1-carboxylate

To a solution of tert-butyl 4-[4-(4-amino-2-chlorobenzoyl)piperazine-1-carbonyl]piperazine-1-carboxylate (90.77 mg, 0.2 mmol, 0.7 eq) and pyridine (453.89 mg, 5.74 mmol, 20.0 eq) in methylene chloride (5 mL) was added a suspension of 5-amino-1-[4-(cyclopropoxy)-2,3-difluoro-phenyl]imidazole-4-carbonyl chloride (90.0 mg, 0.29 mmol, 1.0 eq) in methylene chloride (5 mL) at 25° C. The mixture was stirred at 25° C. for 12 h under N₂. The resulting solution was evaporated under reduced pressure. The residue was purified by column chromatography (silica gel, 0 to 10% MeOH in methylene chloride) to afford tert-butyl 4-[4-[4-[[5-amino-1-[4-(cyclopropoxy)-2,3-difluorophenyl]imidazole-4-carbonyl]amino]-2-chlorobenzoyl]piperazine-1-carbonyl]piperazine-1-carboxylate (121.0 mg, 0.17 mmol, 50.0% yield). MS [M+H]⁺: 728.8

### Step 8: 5-amino-N-[3-chloro-4-[4-(piperazine-1-carbonyl)piperazine-1-carbonyl]phenyl]-1-[4-(cyclopropoxy)-2,3-difluoro-phenyl]imidazole-4-carboxamide; 1:1 2,2,2-trifluoroacetic acid

A solution of tert-butyl 4-[4-[4-[[5-amino-1-[4-(cyclopropoxy)-2,3-difluorophenyl]imidazole-4-carbonyl]amino]-2-chlorobenzoyl]piperazine-1-carbonyl]piperazine-1-carboxylate (101.0 mg, 0.14 mmol, 1.0 eq) in methylene chloride (3 mL) was added trifluoroacetic acid (1.0 mL, 0.14 mmol, 1.0 eq). The reaction was stirred at 25° C. for 1 h. The reaction was concentrated to dryness to afford a crude product. The crude was purified by Prep-HPLC (Gemini-C18 150×21.2 mm, 5 μm, MeCN-H₂O (0.1% TFA), 15%-40%) to afford 5-amino-N-[3-chloro-4-[4-(piperazine-1-carbonyl)piperazine-1-carbonyl]phenyl]-1-[4-(cyclopropoxy)-2,3-difluorophenyl]imidazole-4-carboxamide; 1:1 2,2,2-trifluoroacetic acid (37.5 mg, 0.05 mmol, 36.4% yield). MS [M+H]⁺: 629.0

### Compound 226

### 5-amino-N-[3-chloro-4-[4-(piperazine-1-carbonyl)piperazine-1-carbonyl]phenyl]-1-(4-fluoro-1-methylindol-5-yl)imidazole-4-carboxamide; 1:1 2,2,2-trifluoroacetic acid

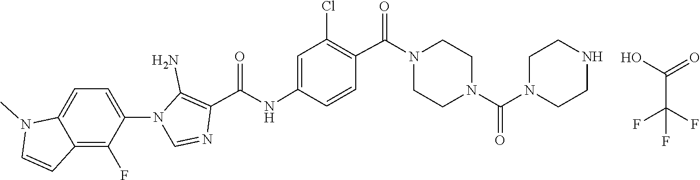 [SMILES: Cn1ccc2c(F)c(-n3cnc(C(=O)Nc4ccc(C(=O)N5CCN(C(=O)N6CCNCC6)CC5)c(Cl)c4)c3N)ccc21 ; O=C(O)C(F)(F)F]

### Step 1: 4-fluoro-1-methyl-5-nitroindole

A solution of 4-fluoro-5-nitro-1H-indole (500.0 mg, 2.78 mmol, 1.0 eq) and iodomethane (0.26 mL, 4.16 mmol, 1.5 eq) in MeCN (5 mL) was stirred at 25° C. for 4 h. The reaction was concentrated to dryness. The residue was taken up in EtOAc (30 mL) and the organics washed with 2×10 mL water followed by 10 mL brine. The organics were then separated and dried (Na₂SO₄) before concentrating to dryness. The crude was then purified by flash column chromatography eluting with 20% EtOAc in petroleum ether to afford 4-fluoro-1-methyl-5-nitro-indole (350.0 mg, 1.8 mmol, 64.9% yield). MS [M+H]⁺: 195.1

### Step 2: 4-fluoro-1-methyl-indol-5-amine

A solution of 4-fluoro-1-methyl-5-nitroindole (300.0 mg, 1.55 mmol, 1.0 eq), iron (431.41 mg, 7.73 mmol, 5.0 eq) and ammonium chloride (413.22 mg, 7.73 mmol, 5.0 eq) in ethanol (3 mL) and water (0.600 mL) was stirred at 50° C. for 2 h. The solid was filtered off. The crude was then purified by flash column chromatography eluting with 40% EtOAc in petroleum ether to give the 4-fluoro-1-methylindol-5-amine (200.0 mg, 1.22 mmol, 78.8% yield). MS [M+H]⁺: 165.1

### Step 3: ethyl 5-amino-1-(4-fluoro-1-methyl-indol-5-yl)imidazole-4-carboxylate

To a solution of ethyl 2-amino-2-cyanoacetate; 4-methylbenzene-1-sulfonic acid (914.64 mg, 3.05 mmol, 2.0 eq) in MeCN (2 mL) were added triethylamine (0.85 mL, 6.09 mmol, 4.0 eq) and triethyl orthoformate (6.7 mL, 40.3 mmol, 26.47 eq). The resulting mixture was stirred at 80° C. for 2 h. The solvent was removed under reduced pressure and the residue was diluted with MeCN (2 mL). Then 4-fluoro-1-methylindol-5-amine (250.0 mg, 1.52 mmol, 1.0 eq) was added and the resulting mixture was stirred at 50° C. for 15 h. After concentration under reduced pressure, the residue was purified by flash column chromatography on silica gel (30:70 petroleum ether:EtOAc) to give ethyl 5-amino-1-(4-fluoro-1-methylindol-5-yl)imidazole-4-carboxylate (300.0 mg, 0.99 mmol, 65.2% yield). MS [M+H]⁺: 302.1

### Step 4: 5-amino-1-(4-fluoro-1-methylindol-5-yl)imidazole-4-carboxylic acid

Sodium hydroxide (211.69 mg, 5.29 mmol, 5.0 eq) was added to a solution of ethyl 5-amino-1-(4-fluoro-1-methylindol-5-yl)imidazole-4-carboxylate (320.0 mg, 1.06 mmol, 1.0 eq) in isopropyl alcohol (4 mL) and water (2 mL). The resulting mixture was stirred at 50° C. for 2 h. The mixture was adjusted to pH 5-6 with 2 N aqueous HCl and extracted with EtOAc. The reaction mixture was partitioned between EtOAc and water. The aqueous layer was extracted again with EtOAc. The combined organic layer was dried over anhydrous sodium sulfate and concentrated under reduced pressure give 5-amino-1-(4-fluoro-1-methylindol-5-yl)imidazole-4-carboxylic acid (140.0 mg, 0.51 mmol, 48.2% yield). MS [M+H]⁺: 275.2

### Step 5: 5-[(E)-dimethylaminomethyleneamino]-1-(4-fluoro-1-methylindol-5-yl)imidazole-4-carbonyl chloride

A solution of 5-amino-1-(4-fluoro-1-methylindol-5-yl)imidazole-4-carboxylic acid (40.0 mg, 0.15 mmol, 1.0 eq), oxalyl chloride (0.01 mL, 0.18 mmol, 1.2 eq) and N,N-dimethylformamide (0.0 mL, 0.01 mmol, 0.1 eq) in methylene chloride (2 mL) was stirred at 25° C. for 1 h. The resulting solution was concentrated under reduced pressure to give 5-[(E)-dimethylaminomethyleneamino]-1-(4-fluoro-1-methylindol-5-yl)imidazole-4-carbonyl chloride (45.0 mg, 0.13 mmol, 88.7% yield). MS [(M−Cl+OCH₃)+H]⁺: 334.0

### Step 6: tert-butyl 4-[4-[2-chloro-4-[[5-[(E)-dimethylaminomethyleneamino]-1-(4-fluoro-1-methyl-indol-5-yl)imidazole-4-carbonyl]amino]benzoyl]piperazine-1-carbonyl]piperazine-1-carboxylate

A solution of 5-[(E)-dimethylaminomethyleneamino]-1-(4-fluoro-1-methyl-indol-5-yl)imidazole-4-carbonyl chloride (45.0 mg, 0.13 mmol, 1.0 eq) and tert-butyl 4-[4-(4-amino-2-chlorobenzoyl)piperazine-1-carbonyl]piperazine-1-carboxylate (40.94 mg, 0.09 mmol, 0.7 eq) in pyridine (0.16 mL, 1.94 mmol, 15.0 eq) was stirred at 25° C. for 1 h. The resulting solution was concentrated under reduced pressure to give tert-butyl 4-[4-[2-chloro-4-[[5-[(E)-dimethylaminomethyleneamino]-1-(4-fluoro-1-methylindol-5-yl)imidazole-4-carbonyl]amino]benzoyl]piperazine-1-carbonyl]piperazine-1-carboxylate (40.0 mg, 0.05 mmol, 40.5% yield). MS [M+H]⁺: 763.3

### Step 7: tert-butyl 4-[4-[4-[[5-amino-1-(4-fluoro-1-methylindol-5-yl)imidazole-4-carbonyl]amino]-2-chlorobenzoyl]piperazine-1-carbonyl]piperazine-1-carboxylate

A solution of tert-butyl 4-[4-[2-chloro-4-[[5-[(E)-dimethylaminomethyleneamino]-1-(4-fluoro-1-methylindol-5-yl)imidazole-4-carbonyl]amino]benzoyl]piperazine-1-carbonyl]piperazine-1-carboxylate (40.0 mg, 0.05 mmol, 1.0 eq) and NaOH (10.48 mg, 0.26 mmol, 5.0 eq) in methanol (1 mL) and water (0.250 mL) was stirred for 1 h at 60° C. The resulting solution was diluted with 50 ml of water, then extracted with 3×40 mL of ethyl acetate. The organic layers were combined, dried and concentrated under reduced pressure to give tert-butyl 4-[4-[4-[[5-amino-1-(4-fluoro-1-methylindol-5-yl)imidazole-4-carbonyl]amino]-2-chlorobenzoyl]piperazine-1-carbonyl]piperazine-1-carboxylate (30.0 mg, 0.04 mmol, 80.8% yield). MS [M+H]⁺: 708.1

### Step 8: 5-amino-N-[3-chloro-4-[4-(piperazine-1-carbonyl)piperazine-1-carbonyl]phenyl]-1-(4-fluoro-1-methylindol-5-yl)imidazole-4-carboxamide; 1:1 2,2,2-trifluoroacetic acid

A solution of tert-butyl 4-[4-[4-[[5-amino-1-(4-fluoro-1-methylindol-5-yl)imidazole-4-carbonyl]amino]-2-chlorobenzoyl]piperazine-1-carbonyl]piperazine-1-carboxylate (30.0 mg, 0.04 mmol, 1.0 eq) and trifluoroacetic acid (1.0 mL, 12.98 mmol, 306.41 eq) in methylene chloride (3 mL) was stirred at 25° C. for 1 h. The resulting solution was concentrated under reduced pressure. The residue was purified by Prep-HPLC (Gemini C18 150×21.2 mm, 5 μm, MeCN-H₂O (0.1% TFA), 25-40) to give 5-amino-N-[3-chloro-4-[4-(piperazine-1-carbonyl)piperazine-1-carbonyl]phenyl]-1-(4-fluoro-1-methylindol-5-yl)imidazole-4-carboxamide; 1:1 2,2,2-trifluoroacetic acid (16.3 mg, 0.02 mmol, 53.2% yield). MS [M+H]⁺: 608.3

### Compound 228

### 5-amino-N-[3-chloro-4-[4-(piperidine-4-carbonyl)piperazine-1-carbonyl]phenyl]-1-[4-(difluoromethoxy)-2,3-difluorophenyl]imidazole-4-carboxamide; formic acid

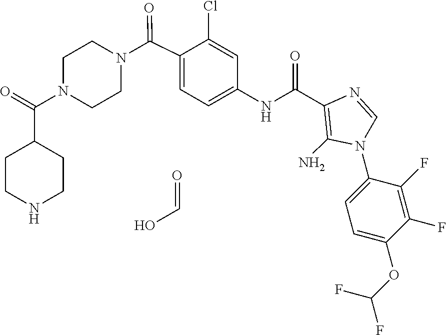 [SMILES: Nc1c(C(=O)Nc2ccc(C(=O)N3CCN(C(=O)C4CCNCC4)CC3)c(Cl)c2)ncn1-c1ccc(OC(F)F)c(F)c1F ; O=CO]

### Step 1: 1-bromo-4-(difluoromethoxy)-2,3-difluoro-benzene

To a solution of 4-bromo-2,3-difluorophenol (100 g, 478.49 mmol, 1.0 eq) in DMF (1000 mL) and H₂O (200 mL) was added sodium chlorodifluoroacetate (145.9 g, 957.0 mmol, 2.0 eq) and K₂CO₃ (79.36 g, 574.19 mmol, 1.2 eq), then the mixture was heated to 100° C. and stirred for 16 h. The mixture was quenched with H₂O (50 mL) and extracted with EtOAc (2×30 mL). The combined organic phase was washed with brine (30 mL), dried over Na₂SO₄, filtered and the filtrate was concentrated under reduced pressure. The residue was purified by column (PE:EtOAc=1:0 to 3:1, Rf=0.65, UV) to afford the title compound (111.0 g, 428.57 mmol, 90% yield) as light-yellow oil. 1H NMR (400 MHz, CDCl₃) δ 7.36-7.28 (m, 1H), 7.04-6.94 (m, 1H), 6.57 (t, J=72.4 Hz, 1H) ppm.

### Step 2: N-[4-(difluoromethoxy)-2,3-difluorophenyl]-1,1-diphenyl-methanimine

A solution of 1-bromo-4-(difluoromethoxy)-2,3-difluoro-benzene (20 g, 77.22 mmol, 1.0 eq), benzenemethanimine, alphaphenyl-(12.96 mL, 77.22 mmol, 1.0 eq), 9,9-dimethyl-4,5-bis(diphenylphosphino) xanthene (2.23 g, 3.86 mmol, 0.05 eq), tris(dibenzylideneacetone)dipalladium (0) (3.54 g, 3.86 mmol, 0.05 eq) and CS₂CO₃ (50.32 g, 154.44 mmol, 2.0 eq) in 1,4-dioxane (200 mL) was stirred at 90° C. for 4 h under N₂. The mixture was filtered and the filtrate was concentrated under reduced pressure. The residue was purified by column (PE:EtOAc 1:0 to 4:1, Rf=0.60, UV) to afford the title compound (18 g, 50.09 mmol, 65% yield) as yellow oil.

### Step 3: tert-butyl 4-[4-[4-[[5-(tert-butoxycarbonylamino)-1-[4-(difluoromethoxy)-2,3-difluorophenyl]imidazole-4-carbonyl]amino]-2-chloro-benzoyl]piperazine-1-carbonyl]piperidine-1-carboxylate

A solution of N-[4-(difluoromethoxy)-2,3-difluorophenyl]-1,1-diphenyl-methanimine (18 g, 50.09 mmol, 1.0 eq) in CH₂Cl₂ (150 mL) was added HCl (12.52 mL, 150.3 mmol, 3.0 eq), the mixture was stirred at 25° C. for 3 h. The mixture was quenched with H₂O (100 mL) and extracted with EtOAc (2×100 mL). The combined organic phase was washed with brine (100 mL), dried over Na₂SO₄ and concentrated under reduced pressure. The residue was purified by column (PE:EtOAc=1:0 to 4:1, Rf=0.40, UV) to afford the title compound (5.4 g, 27.68 mmol, 55% yield) as brown oil. MS: m/z=196.0 [M+H]+, ESI pos.

### Step 4: ethyl 5-amino-1-[4-(difluoromethoxy)-2,3-difluorophenyl]imidazole-4-carboxylate

A mixture of ethyl 2-amino-2-cyanoacetate; 4-methylbenzene-1-sulphonic aci (10.97 g, 36.52 mmol, 1.25 eq), triethylamine (5.09 mL, 36.52 mmol, 1.25 eq) and trimethyl orthoformate (100 mL, 29.21 mmol, 1.0 eq) was stirred at 90° C. for 16 h. The reaction mixture was concentrated under reduced pressure. The residue was dissolved in MeCN (100 mL) and 4-(difluoromethoxy)-2,3-difluoro-aniline (5.7 g, 29.21 mmol, 1.0 eq) was added. The mixture was stirred at 90° C. for another 4 h. The mixture was concentrated under reduced pressure. The residue was purified by reverse phase HPLC (H₂O (0.1% FA)-MeCN, 40-50% B) to afford the title compound (3.2 g, 9.6 mmol, 33% yield) as brown oil. MS: m/z=334.1 [M+H]+, ESI pos.

### Step 5: ethyl 5-[bis(tert-butoxycarbonyl)amino]-1-[4-(difluoromethoxy)-2,3-difluorophenyl]imidazole-4-carboxylate

To a solution of ethyl 5-amino-1-[4-(difluoromethoxy)-2,3-difluorophenyl]imidazole-4-carboxylate (3.2 g, 9.6 mmol, 1.0 eq), DMAP (0.59 g, 4.8 mmol, 0.5 eq) and triethylamine (4.02 mL, 28.81 mmol, 3.0 eq) in CH₂Cl₂ (30 mL) was dropwise added Boc₂O (8.38 g, 38.41 mmol, 4.0 eq) at 0° C. under N₂, then the mixture was stirred at 25° C. for 16 h. The mixture was concentrated under reduced pressure. The residue was purified by reverse phase HPLC (H₂O (0.1% FA)-MeCN, 60-80% B) to afford the title compound (2.5 g, 4.69 mmol, 49% yield) as brown oil. MS: m/z=534.2 [M+H]+, ESI pos.

### Step 6: tert-butyl 4-[4-[4-[[5-(tert-butoxycarbonylamino)-1-[4-(difluoromethoxy)-2,3-difluorophenyl]imidazole-4-carbonyl]amino]-2-chloro-benzoyl]piperazine-1-carbonyl]piperidine-1-carboxylate

To a mixture of tert-butyl 4-[4-(4-amino-2-chloro-benzoyl)piperazine-1-carbonyl]piperidine-1-carboxylate ([2489205-72-1], 250 mg, 0.55 mmol, 1.0 eq) and ethyl 5-[bis(tert-butoxycarbonyl)amino]-1-[4-(difluoromethoxy)-2,3-difluorophenyl]imidazole-4-carboxylate (295 mg, 0.55 mmol, 1.0 eq) in THF (5 mL) was added dropwise lithium bis(trimethylsilyl)amide (2.22 mL, 2.22 mmol, 4.0 eq) at −78° C. under N₂. The resulting solution was then stirred at −78° C. for 1 h. Then the solution was warmed to 20° C. and stirred for another 16 h. The solution was poured into 1M HCl aqueous solution (40 mL), extracted with EtOAc (3×60 mL). The combined organic phase was washed with brine (3×60 mL), dried over Na₂SO₄, filtered and the filtrate was concentrated under reduced pressure. The residue was purified by reverse phase HPLC (H₂O (0.1% FA)-MeCN; 60-80% B) to afford the title compound (230 mg, 0.26 mmol, 49% yield). MS: m/z=738.4 [M−C5H9O2+H]+, ESI pos.

### Step 7: 5-amino-N-[3-chloro-4-[4-(piperidine-4-carbonyl)piperazine-1-carbonyl]phenyl]-1-[4-(difluoromethoxy)-2,3-difluorophenyl]imidazole-4-carboxamide; formic acid

A solution of tert-butyl 4-[4-[4-[[5-(tert-butoxycarbonylamino)-1-[4-(difluoromethoxy)-2,3-difluorophenyl]imidazole-4-carbonyl]amino]-2-chloro-benzoyl]piperazine-1-carbonyl]piperidine-1-carboxylate (180 mg, 0.2 mmol, 1.0 eq) in HCl/dioxane (2.0 mL, 2 M) was stirred at 10° C. for 0.5 h. The mixture was concentrated and purified by prep-HPLC (Instrument: Gilson GX-281+Shimadzu 20AP Pump+SPD-20A Detector, column: Boston Green ODS 150×30 mm×5 μm, mobile phase: A: H₂O (0.225% FA); B: MeCN, 15-45% B in 11 min) to afford the title compound (68.3 mg, 0.1 mmol, 48% yield). MS: m/z=638.3 [M+H]+, ESI pos.

The following compounds were prepared in analogy to the procedure above

| Compound | Reagents | MS [M + H]+ |
|---|---|---|
| 229 | Intermediate 43, | 618.1 |
| 230 | Intermediate 44, | 636.3 |
| 231 | Intermediate 40, | 650.3 |
| | Prep-HPLC (HCl) | |
| 232 | Intermediate 47/With HFIP | 644.1 |
| 233 | Intermediate 41, | 652.3 |
| | tert-butyl 4-[4-(4-amino- | |
| | 2-fluoro-benzoyl)piperazine- | |
| | 1-carbonyl]piperidine-1- | |
| | carboxylate | |
| 234 | Intermediate 42 | 544.1 |
| | Intermediate 125/With | |
| | Prep-HPLC (NH₃•H₂O) | |

### Compound 235

### 5-amino-N-[3-chloro-4-[4-[2-(tetrahydropyran-4-ylamino)-1,4,5,6-tetrahydropyrimidine-5-carbonyl]piperazine-1-carbonyl]phenyl]-1-[2,3-difluoro-4-(fluoromethoxy)phenyl]imidazole-4-carboxamide;2,2,2-trifluoroacetic acid

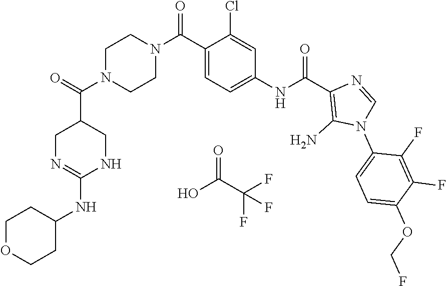 [SMILES: Nc1c(C(=O)Nc2ccc(C(=O)N3CCN(C(=O)C4CN=C(NC5CCOCC5)NC4)CC3)c(Cl)c2)ncn1-c1ccc(OCF)c(F)c1F ; O=C(O)C(F)(F)F]

### Step 1: 5-amino-N-[3-chloro-4-[4-[2-(tetrahydropyran-4-ylamino)-1,4,5,6-tetrahydropyrimidine-5-carbonyl]piperazine-1-carbonyl]phenyl]-1-[2,3-difluoro-4-(fluoromethoxy)phenyl]imidazole-4-carboxamide;2,2,2-trifluoroacetic acid

To a solution of 5-amino-N-[3-chloro-4-(piperazine-1-carbonyl)phenyl]-1-[2,3-difluoro-4-(fluoromethoxy)phenyl]imidazole-4-carboxamide;hydrochloride (Intermediate 70, 50 mg, 0.09 mmol, 1.0 eq), 2-(tetrahydropyran-4-ylamino)-1,4,5,6-tetrahydropyrimidine-5-carboxylic acid;2,2,2-trifluoroacetic acid (Intermediate 90, 125 mg, 0.37 mmol, 4.0 eq) and DIEA (47 mg, 0.37 mmol, 4.0 eq) in DMF (1 mL) was added HATU (69 mg, 0.18 mmol, 2.0 eq) at 20° C., the mixture was stirred at 20° C. for 1 h. The reaction mixture was purified by prep-HPLC (column: Unisil 3-100 C18 Ultra 150×50 mm×3 μm; mobile phase: [H₂O (0.1% FA, V/V)-MeCN]; 10-40% B, 10 min) to afford the title compound (38.2 mg, 0.05 mmol, 48% yield). MS: m/z=718.4 [M+H]+, ESI pos.

### Compound 237

### 5-amino-N-[3-chloro-4-[4-[2-(1-imino-1-oxo-1,4-thiazinan-4-yl)-1,4,5,6-tetrahydropyrimidine-5-carbonyl]piperazine-1-carbonyl]phenyl]-1-[2,3-difluoro-4-(fluoromethoxy)phenyl]imidazole-4-carboxamide;2,2,2-trifluoroacetic acid

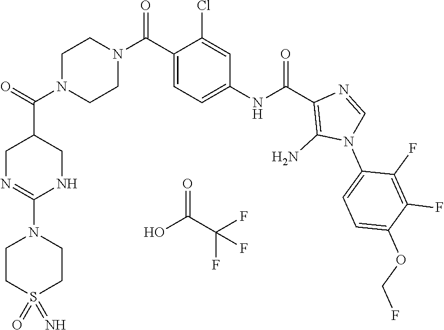 [SMILES: N=S1(=O)CCN(C2=NCC(C(=O)N3CCN(C(=O)c4ccc(NC(=O)c5ncn(-c6ccc(OCF)c(F)c6F)c5N)cc4Cl)CC3)CN2)CC1 ; O=C(O)C(F)(F)F]

### Step 1: methyl 2-thiomorpholinopyrimidine-5-carboxylate

To a mixture of methyl 2-chloropyrimidine-5-carboxylate (5 g, 28.97 mmol, 1.0 eq), K₂CO₃ (4 g, 28.97 mmol, 1.0 eq) in DMF (50 mL) was added thiomorpholine (2.99 g, 28.97 mmol, 1.0 eq), then the mixture was stirred at 30° C. for 16 h. The mixture was poured into H₂O (300 mL) and extracted with EtOAc (2×200 mL), washed with brine (3×400 mL), dried over Na₂SO₄, concentrated to give a residue. The residue was triturated in MTBE (100 mL) and filtered to afford the title compound (6.8 g, 28.42 mmol, 98% yield). MS: m/z=240.0 [M+H]+, ESI pos.

### Step 2: methyl 2-(1-imino-1-oxo-1,4-thiazinan-4-yl)pyrimidine-5-carboxylate

To a mixture of methyl 2-thiomorpholinopyrimidine-5-carboxylate (1.7 g, 7.1 mmol, 1.0 eq) and (diacetoxyiodo)benzene (4.58 g, 14.21 mmol, 2.0 eq) in MeOH (40 mL) was added ammonium carbamate (2.77 g, 35.52 mmol, 5.0 eq) in portions at 30° C. The mixture was then stirred at 30° C. for 2 h. A large quantity of white solid was formed. The mixture was filtered, washed by MeOH (30 mL) and the filter cake was collected and dried to afford the title compound (1360 mg, 5.03 mmol, 71% yield). MS: m/z=271.2 [M+H]+, ESI pos.

### Step 3: 2-(1-imino-1-oxo-1,4-thiazinan-4-yl)pyrimidine-5-carboxylic acid

To a mixture of methyl 2-(1-imino-1-oxo-1,4-thiazinan-4-yl)pyrimidine-5-carboxylate (400 mg, 1.48 mmol, 1.0 eq) in MeOH (6 mL) and H₂O (1.5 mL) was added LiOH·H₂O (68.3 mg, 1.63 mmol, 1.1 eq). The mixture was then stirred at 30° C. for 2 h. The mixture was diluted with MeOH (20 mL) and concentrated under reduced pressure to afford the title compound (379 mg, 1.48 mmol, 99% yield).

MS: m/z=257.1 [M+H]+, ESI pos.

### Step 4: 2-(1-imino-1-oxo-1,4-thiazinan-4-yl)-1,4,5,6-tetrahydropyrimidine-5-carboxylic acid

Solution 1:2-(1-imino-1-oxo-1,4-thiazinan-4-yl)pyrimidine-5-carboxylic acid (426.0 mg, 1.66 mmol, 1.0 eq) in MeOH (40 mL) and trifluoroacetic acid (4.0 mL, 1.66 mmol, 1.0 eq). The fixed bed (named FLR1, volume 5 mL) was completely packed with granular catalyst Pd/Al₂O₃ (3 g, 1.0 eq). The H₂ back pressure regulator was adjusted to 0.5 MPa, and the flow rate of {H2, WuXi-EHS, 30 mL/min}. Then the solution S1 was pumped by Pump 1 {S1, P1, 0.4 mL/min} to fixed bed {FLR1, SS, Fixed bed, 6.350 (¼″) mm, 1 mL, 35° C.}. Then the reaction mixture was collected from the reactor output. The reaction was lasted 3 h. The mixture was concentrated under reduced pressure to afford the title compound (430 mg, 1.65 mmol, 99% yield) as white oil. MS: m/z=261.1 [M+H]+, ESI pos.

### Step 5: 2-(1-imino-1-oxo-1,4-thiazinan-4-yl)-1,4,5,6-tetrahydropyrimidine-5-carboxylic acid

To a solution of 2-(1-imino-1-oxo-1,4-thiazinan-4-yl)-1,4,5,6-tetrahydropyrimidine-5-carboxylic acid (100 mg, 0.38 mmol, 5.24 eq) in DMF (1 mL) was added 5-amino-N-[3-chloro-4-(piperazine-1-carbonyl)phenyl]-1-[2,3-difluoro-4-(fluoromethoxy)phenyl]imidazole-4-carboxamide;hydrochloride (Intermediate 70, 40 mg, 0.07 mmol, 1.0 eq), N,N-diisopropylethylamine (0.04 mL, 0.22 mmol, 3.0 eq) and O-(7-azabenzotriazol-1-yl)-N,N,N′,N′-tetramethyluronium hexafluorophosphate (30 mg, 0.08 mmol, 1.1 eq). The mixture was stirred at 25° C. for 1 h. The reaction mixture was poured into H₂O (20 mL) and extracted with EtOAc (3×30 mL). The combined organic phase was washed by brine (2×40 mL), dried over Na₂SO₄, filtered and concentrated under reduced pressure. The residue was purified by prep-HPLC (column: Waters Xbridge 150×25 mm×10 μm; mobile phase: [H₂O (FA)-MeCN]; 10-40% B, 10 min) to afford the title compound (5.9 mg, 0.01 mmol, 9% yield). MS: m/z=751.1 [M+H]+, ESI pos.

The following compounds were prepared in analogy to the procedure above

| Compound | Reagents | MS [M + H]+ |
|---|---|---|
| 238 | Intermediate 68, | 736.2 |
| | Intermediate 93 | |
| 239 | Intermediate 68, | 706.3 |
| | Intermediate 95 | |
| 240 | Intermediate 68, | 710.2 |
| | Intermediate 98 | |
| 241 | Intermediate 68, | 706.3 |
| | Intermediate 101 | |
| 242 | Intermediate 68, | 720.3 |
| | Intermediate 102, | |
| 243 | Intermediate 70, | 704.3 |
| | Intermediate 87 | |
| 244 | Intermediate 70, | 752.4 |
| | Intermediate 111 | |
| 245 | Intermediate 70, | 718.1 |
| | Intermediate 91 | |
| 246 | Intermediate 70, | 718.2 |
| | Intermediate 92 | |
| 247 | Intermediate 70, | 674.2 |
| | Intermediate 95 | |
| 248 | Intermediate 70, | 690.2 |
| | Intermediate 94 | |
| 249 | Intermediate 70, | 690.2 |
| | Intermediate 89 | |
| 250 | Intermediate 70, | 722.2 |
| | Intermediate 88 | |
| 251 | Intermediate 70, | 678.2 |
| | Intermediate 98 | |
| 252 | Intermediate 70, | 708.3 |
| | Intermediate 112 | |
| 253 | Intermediate 70, | 704.4 |
| | Intermediate 96 | |
| 254 | Intermediate 70, | 734.2 |
| | Intermediate 99 | |
| 255 | Intermediate 70, | 767.2 |
| | Intermediate 100 | |
| 256 | Intermediate 70, | 752.2 |
| | Intermediate 103 | |
| 257 | Intermediate 70, | 662.2 |
| | Intermediate 110 | |
| 258 | Intermediate 70, | 767.2 |
| | Intermediate 104 | |
| 259 | Intermediate 70, | 716.2 |
| | Intermediate 113 | |
| 260 | Intermediate 70, | 764.1 |
| | Intermediate 105 | |
| 261 | Intermediate 70, | 778.2 |
| | Intermediate 106 | |
| 262 | Intermediate 70, | 750.2 |
| | Intermediate 107 | |
| 263 | Intermediate 70, | 793.5 |
| | Intermediate 115 | |
| 264 | Intermediate 70, | 738.3 |
| | Intermediate 116 | |
| 265 | Intermediate 70, | 740.3 |
| | Intermediate 114 | |
| 266 | Intermediate 70, | 755.2 |
| | Intermediate 117 | |
| 267 | Intermediate 70, | 733.2 |
| | Intermediate 108 | |
| 268 | Intermediate 70, | 726.2 |
| | Intermediate 119/With BBDI | |
| 269 | Intermediate 70, | 733.2 |
| | Intermediate 109 | |
| 270 | Intermediate 70, | 758.3 |
| | Intermediate 123 | |
| 271 | Intermediate 68, | 680.2 |
| | 1-methyl-4- | |
| | piperidineacetic acid | |
| 272 | Intermediate 68, | 678.2 |
| | 2-[(1R,5S)-3-methyl-3- | |
| | azabicyclo[3.1.0]hexan- | |
| | 6-yl]acetic acid | |
| 273 | Intermediate 72 | 724.2 |
| | Intermediate 112 | |
| 274 | Intermediate 73 | 708.3 |
| | Intermediate 112 | |

### Compound 275

### 5-amino-N-[3-chloro-4-[4-(1,4,5,6-tetrahydropyrimidine-5-carbonyl)piperazine-1-carbonyl]phenyl]-1-[2,3-difluoro-4-(fluoromethoxy)phenyl]imidazole-4-carboxamide; bis(2,2,2-trifluoroacetic acid)

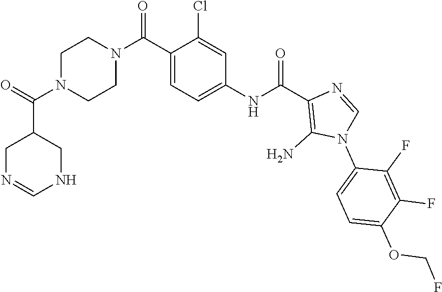 [SMILES: Nc1c(C(=O)Nc2ccc(C(=O)N3CCN(C(=O)C4CN=CNC4)CC3)c(Cl)c2)ncn1-c1ccc(OCF)c(F)c1F]

To a mixture of 4-[[5-amino-1-[2,3-difluoro-4-(fluoromethoxy)phenyl]imidazole-4-carbonyl]amino]-2-chloro-benzoic acid;hydrochloride (Intermediate 74, 100 mg, 0.21 mmol, 1.0 eq) and N-ethyl-N-isopropylpropan-2-amine (162 mg, 1.26 mmol, 6.0 eq) in DMSO (2 mL) was added HATU (79 mg, 0.21 mmol, 1.0 eq). The reaction mixture was stirred at 25° C. for 0.25 h. Then, piperazin-1-yl(1,4,5,6-tetrahydropyrimidin-5-yl) methanone;dihydrochloride (112 mg, 0.42 mmol, 2.0 eq) was added. The resulting mixture was stirred was stirred at 25° C. for another 0.25 h. The reaction mixture was adjust to pH=6 with TFA. The mixture was purified by reverse phase HPLC (H₂O (TFA)-MeCN; 10-35%) and prep-HPLC (column: Phenomenex Luna C18 150×25 mm×10 μm; mobile phase: [H₂O (0.1% TFA)/MeCN]; 3-33% B, 15 min) to afford the title compound (11.9 mg, 0.01 mmol, 9% yield). MS: m/z=619.1 [M+H]+, ESI pos.

The following compounds were prepared in analogy to the procedure above

| Compound | Reagents | MS [M + H]+ |
|---|---|---|
| 276 | Intermediate 74, | 663.1 |
| | Intermediate 66 | |
| 277 | Intermediate 74, | 676.2 |
| | Intermediate 124 | |
| 278 | Intermediate 75, | 776.3 |
| | Intermediate 122 | |

### Compound 279

### 5-amino-N-[3-chloro-4-[4-[2-(oxetan-3-ylmethylamino)-1,4,5,6-tetrahydropyrimidine-5-carbonyl]piperazine-1-carbonyl]phenyl]-1-[2,3-difluoro-4-(fluoromethoxy)phenyl]imidazole-4-carboxamide;hydrochloride

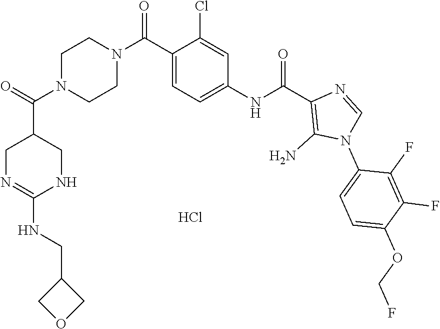 [SMILES: Nc1c(C(=O)Nc2ccc(C(=O)N3CCN(C(=O)C4CN=C(NCC5COC5)NC4)CC3)c(Cl)c2)ncn1-c1ccc(OCF)c(F)c1F ; Cl]

### Step 1: tert-butyl N-[5-[4-[4-[[5-amino-1-[2,3-difluoro-4-(fluoromethoxy)phenyl]imidazole-4-carbonyl]amino]-2-chloro-benzoyl]piperazine-1-carbonyl]-1,4,5,6-tetrahydropyrimidin-2-yl]-N-(oxetan-3-ylmethyl) carbamate

To a stirred solution of 2-[tert-butoxycarbonyl(oxetan-3-ylmethyl)amino]-1,4,5,6-tetrahydropyrimidine-5-carboxylic acid (Intermediate 118, 73 mg, 0.24 mmol, 3.0 eq), 5-amino-N-[3-chloro-4-(piperazine-1-carbonyl)phenyl]-1-[2,3-difluoro-4-(fluoromethoxy)phenyl]imidazole-4-carboxamide;hydrochloride (Intermediate 70, 42 mg, 0.08 mmol, 1.0 eq) and N,N-diisopropylethylamine (0.04 mL, 0.24 mmol, 3.0 eq) in DMF (4 mL) was added HATU (29 mg, 0.08 mmol, 1.0 eq) in one portion at 25° C. The solution was then stirred at 25° C. for 1 h. The mixture was purified by prep-HPLC (Waters Xbridge 150×25 mm×5 μm; mobile phase: [H₂O (FA)-MeCN], 18-48% B, 10 min) to afford the title compound (35 mg, 0.04 mmol, 55% yield). MS: m/z=704.2 [M−C5H9O2+H]+, ESI pos.

### Step 2: 5-amino-N-[3-chloro-4-[4-[2-(oxetan-3-ylmethylamino)-1,4,5,6-tetrahydropyrimidine-5-carbonyl]piperazine-1-carbonyl]phenyl]-1-[2,3-difluoro-4-(fluoromethoxy)phenyl]imidazole-4-carboxamide;hydrochloride

A solution of tert-butyl N-[5-[4-[4-[[5-amino-1-[2,3-difluoro-4-(fluoromethoxy)phenyl]imidazole-4-carbonyl]amino]-2-chloro-benzoyl]piperazine-1-carbonyl]-1,4,5,6-tetrahydropyrimidin-2-yl]-N-(oxetan-3-ylmethyl) carbamate (20 mg, 0.02 mmol, 1.0 eq) in HFIP (2.0 mL, 0.02 mmol, 1.0 eq) was stirred at 80° C. for 16 h. The solution was concentrated under reduced pressure. The residue was purified by prep-HPLC (Waters Xbridge 150×25 mm×5 μm; mobile phase: [H₂O (HCl)-MeCN], 18-48% B, 10 min) to afford the title compound (10.7 mg, 0.01 mmol, 57% yield). MS: m/z=704.2 [M+H]+, ESI pos.

### Compound 280

### 5-amino-N-[3-chloro-4-[4-(1,2,3,5,6,7-hexahydroimidazo[1,2-a]pyrimidine-6-carbonyl)piperazine-1-carbonyl]phenyl]-1-[2,3-difluoro-4-(fluoromethoxy)phenyl]imidazole-4-carboxamide;hydrochloride

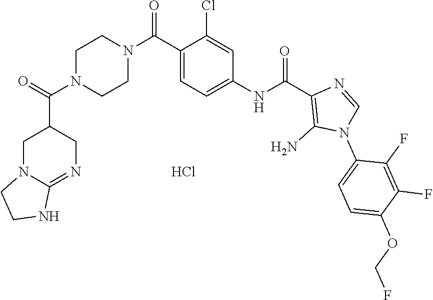 [SMILES: Nc1c(C(=O)Nc2ccc(C(=O)N3CCN(C(=O)C4CN=C5NCCN5C4)CC3)c(Cl)c2)ncn1-c1ccc(OCF)c(F)c1F ; Cl]

### Step 1: methyl 2-(2-hydroxyethylamino)pyrimidine-5-carboxylate

To a solution of methyl 2-chloropyrimidine-5-carboxylate (5 g, 28.97 mmol, 1.0 eq) in MeCN (40 mL) was added 2-aminoethanol (1.77 g, 28.97 mmol, 1.0 eq) and K₂CO₃ (4 g, 28.97 mmol, 1.0 eq), the mixture was stirred at 25° C. for 16 h. The mixture was poured into H₂O (100 mL) and extracted with EtOAc (3×60 mL). The combined organic phase was washed with brine (3×30 mL), dried with Na₂SO₄, filtered and concentrated under reduced pressure, the residue was purified by column (SiO₂, PE:EtOAc=2:1 to 1:1) to afford the title compound (3.8 g, 19.27 mmol, 66% yield). MS: m/z=198.1 [M+H]+, ESI pos.

### Step 2: methyl 2-[tert-butoxycarbonyl(2-tert-butoxycarbonyloxyethyl)amino]pyrimidine-5-carboxylate

To a solution of methyl 2-(2-hydroxyethylamino)pyrimidine-5-carboxylate (3.5 g, 17.75 mmol, 1.0 eq), 4-dimethylaminopyridine (0.22 g, 1.77 mmol, 0.1 eq) and triethylamine (4.95 mL, 35.5 mmol, 2.0 eq) in THF (30 mL) was added BOC₂O (5.81 g, 26.62 mmol, 1.5 eq). The mixture was stirred at 25° C. for 16 h. The mixture was poured into H₂O (60 mL) and extracted with EtOAc (3×60 mL). The combined organic phase was washed with brine (3×50 mL), dried with Na₂SO₄, filtered and concentrated under reduced pressure, the residue was purified by column (SiO₂, PE:EtOAc=5:1 to 3:1) to afford the title compound (4.2 g, 10.57 mmol, 60% yield). MS: m/z=398.1 [M+H]+, ESI pos.

### Step 3: 1-tert-butoxycarbonyl-3,5,6,7-tetrahydro-2H-imidazo[1,2-a]pyrimidine-6-carboxylic acid

To a solution of methyl 2-[tert-butoxycarbonyl(2-tert-butoxycarbonyloxyethyl)amino]pyrimidine-5-carboxylate (2 g, 5.03 mmol, 1.0 eq) in MeOH (30 mL) and H₂O (5 mL) was added 1,1,2-trichloroethane (0.67 g, 5.03 mmol, 1.0 eq) and Pd/SiO₂ (2 g, 1.0 eq), the mixture was stirred at 25° C. for 16 h under H₂ (in balloon). The reaction mixture was filtered, and the filtrate was adjusted to pH 7 with saturated NaHCO₃ aqueous solution. The resulting mixture was concentrated under reduced pressure, the residue was purified by reverse phase HPLC (H₂O-MeCN; 55-65% B) to afford the title compound (150 mg, 0.56 mmol, 10% yield). MS: m/z=270.0 [M+H]+, ESI pos.

### Step 4: tert-butyl 6-[4-[4-[[5-amino-1-[2,3-difluoro-4-(fluoromethoxy)phenyl]imidazole-4-carbonyl]amino]-2-chloro-benzoyl]piperazine-1-carbonyl]-3,5,6,7-tetrahydro-2H-imidazo[1,2-a]pyrimidine-1-carboxylate

To a solution of 1-tert-butoxycarbonyl-3,5,6,7-tetrahydro-2H-imidazo[1,2-a]pyrimidine-6-carboxylic acid (80 mg, 0.3 mmol, 1.0 eq) in DMF (3 mL) was added 5-amino-N-[3-chloro-4-(PIPERAZINE-1-CARBONYL)PHENYL]-1-[2,3-DIFLUORO-4-(FLUOROMETHOXY)PHENYL]IMIDAZOLE-4-carboxamide;hydrochloride (Intermediate 70, 162 mg, 0.3 mmol, 1.0 eq), N,N-diisopropylethylamine (0.16 mL, 0.89 mmol, 3.0 eq) and O-(7-azabenzotriazol-1-yl)-N,N,N′,N′-tetramethyluronium hexafluorophosphate (169 mg, 0.45 mmol, 1.5 eq), the mixture was stirred at 25° C. for 1 h. The mixture was purified by reverse phase HPLC (H₂O (0.1% FA)-MeCN; 60-65% B) to afford the title compound (31.0 mg, 0.04 mmol, 14% yield). MS: m/z=760.3 [M+H]+, ESI pos.

### Step 5: 5-amino-N-[3-chloro-4-[4-(1,2,3,5,6,7-hexahydroimidazo[1,2-a]pyrimidine-6-carbonyl)piperazine-1-carbonyl]phenyl]-1-[2,3-difluoro-4-(fluoromethoxy)phenyl]imidazole-4-carboxamide;hydrochloride

To a solution of tert-butyl 6-[4-[4-[[5-amino-1-[2,3-difluoro-4-(fluoromethoxy)phenyl]imidazole-4-carbonyl]amino]-2-chloro-benzoyl]piperazine-1-carbonyl]-3,5,6,7-tetrahydro-2H-imidazo[1,2-a]pyrimidine-1-carboxylate (15 mg, 0.02 mmol, 1.0 eq) in CH₂Cl₂ (1 mL) was added hydrogen chloride in 1,4-dioxane (0.1 mL, 0.2 mmol, 10.14 eq) and stirred at 25° C. for 0.5 h. The mixture was concentrated under reduced pressure, the residue was purified by prep-HPLC (column: Phenomenex luna C18 150×25 mm×10 um; mobile phase: [A: H₂O (0.05% HCl); B: MeCN]; 12-42% B, 10 min) to afford the title compound (11 mg, 0.02 mmol, 78% yield). MS: m/z=660.3 [M+H]+, ESI pos.

### Compound 281

### 5-amino-1-[1-(2-chloroallyl)-3-(trifluoromethyl)pyrazol-4-yl]-N-[3-chloro-4-[4-[(3R)-3-fluoropyrrolidine-3-carbonyl]piperazine-1-carbonyl]phenyl]imidazole-4-carboxamide;2,2,2-trifluoroacetic acid

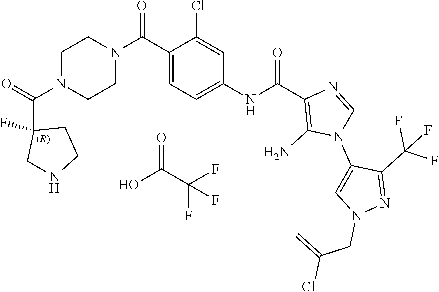

### Step 1: tert-butyl(3R)-3-[4-[4-[[5-amino-1-[3-(trifluoromethyl)-1H-pyrazol-4-yl]imidazole-4-carbonyl]amino]-2-chloro-benzoyl]piperazine-1-carbonyl]-3-fluoro-pyrrolidine-1-carboxylate

To a stirred solution of (3R)-1-tert-butoxycarbonyl-3-fluoro-pyrrolidine-3-carboxylic acid (211 mg, 0.45 mmol, 1.5 eq), 5-amino-N-[3-chloro-4-(piperazine-1-carbonyl)phenyl]-1-[3-(trifluoromethyl)-1H-pyrazol-4-yl]imidazole-4-carboxamide; formic acid (Intermediate 77, 160 mg, 0.3 mmol, 1.0 eq) and N,N-diisopropylethylamine (0.16 mL, 0.91 mmol, 3.0 eq) in DMF (3 mL) was added HATU (172 mg, 0.45 mmol, 1.5 eq) in one portion at 20° C. Then the solution was stirred at 20° C. for 1 h. The reaction mixture was concentrated under reduced pressure. The residue was purified by reverse phase HPLC (0.1% formic acid condition/MeCN, B %: 40-60%) to afford the title compound (114 mg, 0.16 mmol, 53% yield). MS: m/z=642.1 [M−C4H9+H]+, ESI pos.

### Step 2: tert-butyl(3R)-3-[4-[4-[[5-amino-1-[1-(2-chloroallyl)-3-(trifluoromethyl)pyrazol-4-yl]imidazole-4-carbonyl]amino]-2-chloro-benzoyl]piperazine-1-carbonyl]-3-fluoro-pyrrolidine-1-carboxylate

To a solution tert-butyl(3R)-3-[4-[4-[[5-amino-1-[3-(trifluoromethyl)-1H-pyrazol-4-yl]imidazole-4-carbonyl]amino]-2-chloro-benzoyl]piperazine-1-carbonyl]-3-fluoro-pyrrolidine-1-carboxylate (114 mg, 0.16 mmol, 1.0 eq) and K₂CO₃ (67 mg, 0.49 mmol, 3.0 eq) in DMF (5 mL) was added 2,3-dichloro-1-propene (36 mg, 0.33 mmol, 2.0 eq). The mixture was stirred at 20° C. for 16 h. The reaction mixture was concentrated under reduced pressure. The residue was purified by reverse phase HPLC (0.1% formic acid condition/MeCN, B %: 60-80%) and prep-HPLC (column: Welch ultimate XB-NH2 250×50×10 um, mobile phase: condition: (0.1% Heptane)-EtOH; B: 15-55%) to afford the title compound (90 mg, 0.12 mmol, 71% yield). MS: m/z=774.2 [M+H]+, ESI pos.

### Step 3: 5-amino-1-[1-(2-chloroallyl)-3-(trifluoromethyl)pyrazol-4-yl]-N-[3-chloro-4-[4-[(3R)-3-fluoropyrrolidine-3-carbonyl]piperazine-1-carbonyl]phenyl]imidazole-4-carboxamide;2,2,2-trifluoroacetic acid

To a mixture of tert-butyl(3R)-3-[4-[4-[[5-amino-1-[1-(2-chloroallyl)-3-(trifluoromethyl)pyrazol-4-yl]imidazole-4-carbonyl]amino]-2-chloro-benzoyl]piperazine-1-carbonyl]-3-fluoro-pyrrolidine-1-carboxylate (90 mg, 0.12 mmol, 1.0 eq) in CH₂Cl₂ (1 mL) was added TFA (1.0 mL, 1.16 mmol, 10.0 eq) and stirred at 25° C. for 1 h. The reaction mixture was concentrated under reduced pressure. The residue was purified by prep-HPLC (Phenomenex luna C18 150×25 mm×10 um; mobile phase: [H₂O (0.1% TFA)-MeCN]; 10-40% B, 10 min) to afford the title compound (57.3 mg, 0.07 mmol, 62% yield). MS: m/z=672.1 [M+H]+, ESI pos.

The following compounds were prepared in analogy to the procedure above

| | | MS |
|---|---|---|
| Compound | Reagents | [M + H]+ |
| 282 | Intermediate 78 | 656.1 |
| | (R)-1-(tert-butoxycarbonyl)-3- | |
| | fluoropyrrolidine-3-carboxylic acid, | |
| | 2,3-dichloro-1-propene | |
| 283 | Intermediate 77, | 720.2 |
| | BOC-4-fluoro-4-piperidinecarboxylic acid, | |
| | 2-(bromomethyl)-3,3,3-trifluoro-prop-1-ene | |

### Compound 284

### 5-amino-1-[1-(2-chloroallyl)-3-(trifluoromethyl)pyrazol-4-yl]-N-[3-fluoro-4-[4-[(1S,5R)-3-methyl-3-azabicyclo[3.1.0]hexane-6-carbonyl]piperazine-1-carbonyl]phenyl]imidazole-4-carboxamide; hydrochloride

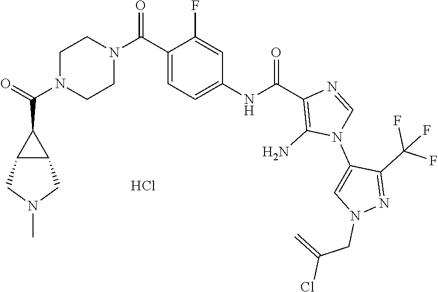 [SMILES: C=C(Cl)Cn1cc(-n2cnc(C(=O)Nc3ccc(C(=O)N4CCN(C(=O)[C@H]5[C@@H]6CN(C)C[C@@H]65)CC4)c(F)c3)c2N)c(C(F)(F)F)n1 ; Cl]

### Step 1: tert-butyl(1S,5R)-6-(4-benzyloxycarbonylpiperazine-1-carbonyl)-3-azabicyclo[3.1.0]hexane-3-carboxylate

To a solution of 1-CBZ-piperazine (2.13 g, 9.68 mmol, 1.1 eq), N,N-diisopropylethylamine (4.18 mL, 24.0 mmol, 2.73 eq) and (1S,5R)-3-tert-butoxycarbonyl-3-azabicyclo[3.1.0]hexane-6-carboxylic acid (2 g, 8.8 mmol, 1.0 eq) in DMF (25 mL) was added HATU (2.82 g, 12.0 mmol, 1.36 eq) at 10° C., the mixture was stirred at 10° C. for 1 h. The mixture was diluted with 1M HCl aqueous solution (50 mL) and extracted with EtOAc (3×50 mL). The combined organic phase was washed with brine (3×300 mL), dried over Na₂SO₄, filtered and concentrated under reduced pressure. The residue was purified by column chromatography (SiO2, PE:EtOAc=2:1 to: 1:3) to afford the title compound (3.44 g, 8.01 mmol, 90% yield). MS: m/z=430.3 [M+H]+, ESI pos.

### Step 2: benzyl 4-[(1S,5R)-3-azabicyclo[3.1.0]hexane-6-carbonyl]piperazine-1-carboxylate;2,2,2-trifluoroacetic acid

To a solution of tert-butyl(1S,5R)-6-(4-benzyloxycarbonylpiperazine-1-carbonyl)-3-azabicyclo[3.1.0]hexane-3-carboxylate (1 g, 2.33 mmol, 1.0 eq) in CH₂Cl₂ (10 mL) was added TFA (4.0 mL, 51.92 mmol, 22.3 eq). The mixture was stirred at 15° C. for 1 h. The mixture was concentrated under reduced pressure to afford the title compound (700 mg, 1.58 mmol, 87% yield) as yellow oil. MS: m/z=330.0 [M+H]+, ESI pos.

### Step 3: benzyl 4-[(1S,5R)-3-methyl-3-azabicyclo[3.1.0]hexane-6-carbonyl]piperazine-1-carboxylate

To a solution of benzyl 4-[(1S,5R)-3-azabicyclo[3.1.0]hexane-6-carbonyl]piperazine-1-carboxylate;2,2,2-trifluoroacetic acid (700 mg, 1.58 mmol, 1.0 eq) in MeOH (10 mL) was added acetic acid (9 mg, 0.16 mmol, 0.1 eq) and formaldehyde (47 mg, 1.58 mmol, 1.0 eq) followed by sodium triacetoxyborohydride (501 mg, 2.37 mmol, 1.5 eq), the mixture was stirred at 20° C. for 2 h. The reaction mixture was concentrated under reduced pressure. The residue was purified by reverse phase HPLC (H₂O (0.1% FA)-MeCN; 50-55% B) to afford the title compound (540 mg, 1.57 mmol, 80% yield). MS: m/z=344.3 [M+H]+, ESI pos.

### Step 4: [(1S,5R)-3-methyl-3-azabicyclo[3.1.0]hexan-6-yl]-piperazin-1-yl-methanone

To a solution of benzyl 4-[(1S,5R)-3-methyl-3-azabicyclo[3.1.0]hexane-6-carbonyl]piperazine-1-carboxylate (500 mg, 1.46 mmol, 1.0 eq) in MeOH (20 mL) was added Pd/Al₂O₃ (3 g, 1.0 eq). The suspension was degassed and purged with H₂ several times. The mixture was stirred at 45° C. for 0.5 h under H₂ (45 psi). The reaction mixture was filtered and washed with MeOH (3×5 mL), the filtrate was concentrated under reduced pressure to afford the title compound (300 mg, 1.43 mmol, 100% yield) as colorless oil. MS: m/z=210.1 [M+H]+, ESI pos.

### Step 5: 5-amino-N-[3-fluoro-4-[4-[(1S,5R)-3-methyl-3-azabicyclo[3.1.0]hexane-6-carbonyl]piperazine-1-carbonyl]phenyl]-1-[3-(trifluoromethyl)-1H-pyrazol-4-yl]imidazole-4-carboxamide;hydrochloride

To a solution of 4-[[5-amino-1-[3-(trifluoromethyl)-1H-pyrazol-4-yl]imidazole-4-carbonyl]amino]-2-fluoro-benzoic acid;hydrochloride (79, 160 mg, 0.37 mmol, 1.0 eq), [(1S,5R)-3-methyl-3-azabicyclo[3.1.0]hexan-6-yl]-piperazin-1-yl-methanone (77 mg, 0.37 mmol, 1.0 eq) and N,N-diisopropylethylamine (0.19 mL, 1.1 mmol, 3.0 eq) in DMF (1.5 mL) was added O-(7-azabenzotriazol-1-yl)-N,N,N′,N′-tetramethyluronium hexafluorophosphate (139 mg, 0.37 mmol, 1.0 eq). The reaction mixture was stirred at 20° C. for 2 h. The mixture was purified by reverse phase HPLC (H₂O (0.1% HCl)-MeCN]; 60-70% B) to afford the title compound (150 mg, 0.24 mmol, 65% yield). MS: m/z=590.3 [M+H]+, ESI pos.

### Step 6: 5-amino-1-[1-(2-chloroallyl)-3-(trifluoromethyl)pyrazol-4-yl]-N-[3-fluoro-4-[4-[(1S,5R)-3-methyl-3-azabicyclo[3.1.0]hexane-6-carbonyl]piperazine-1-carbonyl]phenyl]imidazole-4-carboxamide;hydrochloride

To a solution 5-amino-N-[3-fluoro-4-[4-[(1S,5R)-3-methyl-3-azabicyclo[3.1.0]hexane-6-carbonyl]piperazine-1-carbonyl]phenyl]-1-[3-(trifluoromethyl)-1H-pyrazol-4-yl]imidazole-4-carboxamide;hydrochloride (70.0 mg, 0.11 mmol, 1.0 eq) and K₂CO₃ (46 mg, 0.34 mmol, 3.0 eq) in MeCN (2 mL) was added 2,3-dichloro-1-propene (14 mg, 0.13 mmol, 1.2 eq). The mixture was stirred at 20° C. for 16 h. The mixture was purified by prep-HPLC (column: Waters Xbridge 150×25 mm×5 μm; mobile phase: [H₂O (HCl)-MeCN]; 16-46% B) to afford the title compound (18.3 mg, 0.03 mmol, 23% yield). MS: m/z=664.3 [M+H]+, ESI pos.

The following compounds were prepared in analogy to the procedure above

| Compound | Reagents | MS |
|---|---|---|
| 285 | 2-fluoroallyl 4-methylbenzenesulfonate | 648.2 |

### Compound 286

### 5-amino-N-[3-fluoro-4-[4-[(1S,5R)-3-methyl-3-azabicyclo[3.1.0]hexane-6-carbonyl]piperazine-1-carbonyl]phenyl]-1-[1-[(2S)-2-fluoropropyl]-3-(trifluoromethyl)pyrazol-4-yl]imidazole-4-carboxamide; formic acid

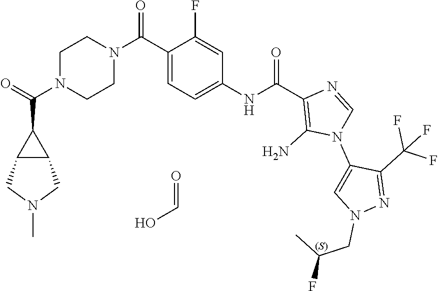

### Step 1: 5-amino-N-[3-fluoro-4-[4-[(1S,5R)-3-methyl-3-azabicyclo[3.1.0]hexane-6-carbonyl]piperazine-1-carbonyl]phenyl]-1-[3-(trifluoromethyl)-1H-pyrazol-4-yl]imidazole-4-carboxamide; hydrochloride

To a solution of 5-amino-N-[3-fluoro-4-(piperazine-1-carbonyl)phenyl]-1-[3-(trifluoromethyl)-1H-pyrazol-4-yl]imidazole-4-carboxamide; dihydrochloride (Intermediate 78, 500 mg, 0.78 mmol, 1.0 eq), (1S,5R)-3-methyl-3-azabicyclo[3.1.0]hexane-6-carboxylic acid;hydrochloride (138 mg, 0.78 mmol, 1.0 eq) and N,N-diisopropylethylamine (0.41 mL, 2.34 mmol, 3.0 eq) in DMF (5 mL), was added HATU (355 mg, 0.93 mmol, 1.2 eq). The reaction was then stirred at 20° C. for 0.5 h. The solution was purified by reverse phase HPLC (H₂O (0.1% HCl)-MeCN]; 40-50% B) to afford the title compound (400 mg, 0.64 mmol, 82% yield). MS: m/z=590.3 [M+H]+, ESI pos.

### Step 2: 5-amino-N-[3-fluoro-4-[4-[(1S,5R)-3-methyl-3-azabicyclo[3.1.0]hexane-6-carbonyl]piperazine-1-carbonyl]phenyl]-1-[1-[(2R)-2-hydroxypropyl]-3-(trifluoromethyl)pyrazol-4-yl]imidazole-4-carboxamide

To a mixture of 5-amino-N-[3-fluoro-4-[4-[(1S,5R)-3-methyl-3-azabicyclo[3.1.0]hexane-6-carbonyl]piperazine-1-carbonyl]phenyl]-1-[3-(trifluoromethyl)-1H-pyrazol-4-yl]imidazole-4-carboxamide;hydrochloride (180.0 mg, 0.29 mmol, 1.0 eq) and K₂CO₃ (119 mg, 0.86 mmol, 3.0 eq) in DMF (3 mL) was dropwise added (R)-(+)-propylene oxide (167 mg, 2.88 mmol, 10.0 eq) at 20° C. The mixture was then stirred at 50° C. for 16 h. The mixture was diluted with H₂O (100 mL) and extracted with EtOAc (3×30 mL). The combined organic phase was washed with brine (3×20 mL), dried over Na₂SO₄, filtered and concentrated under reduced pressure. The residue was purified by prep-HPLC (column: Welch ultimate XB-SiOH 250×50×10 um; mobile phase: [Hexane-EtOH]; 20-60% B over 15 min) to afford the title compound (70 mg, 0.11 mmol, 38% yield). MS: m/z=648.3 [M+H]+, ESI pos.

### Step 3: 5-amino-N-[3-fluoro-4-[4-[(1S,5R)-3-methyl-3-azabicyclo[3.1.0]hexane-6-carbonyl]piperazine-1-carbonyl]phenyl]-1-[1-[(2S)-2-fluoropropyl]-3-(trifluoromethyl)pyrazol-4-yl]imidazole-4-carboxamide; formic acid

To a solution of 5-amino-N-[3-fluoro-4-[4-[(1S,5R)-3-methyl-3-azabicyclo[3.1.0]hexane-6-carbonyl]piperazine-1-carbonyl]phenyl]-1-[1-[(2R)-2-hydroxypropyl]-3-(trifluoromethyl)pyrazol-4-yl]imidazole-4-carboxamide (60 mg, 0.09 mmol, 1.0 eq) in CH₂Cl₂ (2 mL) was added DAST (44.8 mg, 0.28 mmol, 3.0 eq) at 0° C. The mixture was then stirred at 25° C. for 2 h. The mixture was concentrated under reduced pressure. The residue was purified by prep-HPLC (Phenomenex luna C18 150×25 mm×10 um; mobile phase: [H₂O (FA)-MeCN]; 10-40% B, 11 min) to afford the title compound (12.3 mg, 0.02 mmol, 19% yield). MS: m/z=650.5 [M+H]+, ESI pos.

The following compounds were prepared in analogy to the procedure above

| | | MS |
|---|---|---|
| Compound | Reagents | [M + H]+ |
| 287 | Intermediate 77, | 666.3 |
| | (1S,5R)-3-methyl-3-azabicyclo[3.1.0] | |
| | hexane-6-carboxylic acid;hydrochloride | |
| 288 | Intermediate 77, | 652.5 |
| | rac-(1S,5R)-3-tert-butoxycarbonyl-3- | |
| | azabicyclo[3.1.0]hexane-6-carboxylic acid | |
| | with HCl/dioxane | |

### Compound 289

### 5-amino-N-[3-chloro-4-[4-(2-iminohexahydropyrimidine-5-carbonyl)piperazine-1-carbonyl]phenyl]-1-[1-(cyclopropylmethyl)-3-(trifluoromethyl)pyrazol-4-yl]imidazole-4-carboxamide;2,2,2-trifluoroacetic acid

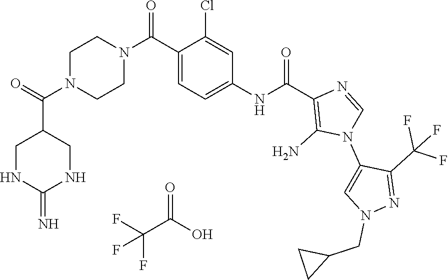 [SMILES: N=C1NCC(C(=O)N2CCN(C(=O)c3ccc(NC(=O)c4ncn(-c5cn(CC6CC6)nc5C(F)(F)F)c4N)cc3Cl)CC2)CN1 ; O=C(O)C(F)(F)F]

To a solution of 5-amino-N-[3-chloro-4-(piperazine-1-carbonyl)phenyl]-1-[1-(cyclopropylmethyl)-3-(trifluoromethyl)pyrazol-4-yl]imidazole-4-carboxamide;hydrochloride (Intermediate 83, 100 mg, 0.17 mmol, 1.0 eq) N,N-diisopropylethylamine (0.09 mL, 0.52 mmol, 3.0 eq), and 2-iminohexahydropyrimidine-5-carboxylic acid (74.9 mg, 0.52 mmol, 3.0 eq) in DMF (2 mL) was HATU (66 mg, 0.17 mmol, 1.0 eq) at 25° C. The mixture was then stirred at 25° C. for 0.5 h. The reaction was purified by prep-HPLC (Phenomenex luna C18 150×25 mm×10 um; mobile phase: [H₂O (FA-MeCN]; 30-40% B, 9 min) to afford the title compound (70.6 mg, 0.09 mmol, 51% yield). MS: m/z=662.1 [M+H]+, ESI pos.

The following compound was prepared in analogy to the procedure above

| Compound | Reagents | MS [M + H]+ |
|---|---|---|
| 290 | Intermediate 84 | 684.4 |

### Compound 291

### 5-amino-N-[3-chloro-4-[4-[rac-(1S,5R)-3-(3-hydroxypropyl)-3-azabicyclo[3.1.0]hexane-6-carbonyl]piperazine-1-carbonyl]phenyl]-1-[1-(cyclobutylmethyl)-3-(trifluoromethyl)pyrazol-4-yl]imidazole-4-carboxamide;hydrochloride

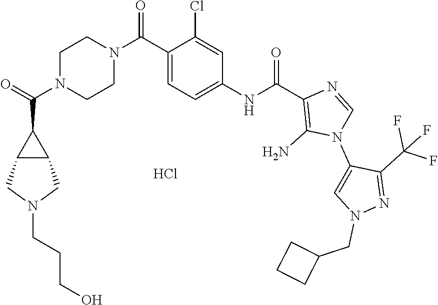 [SMILES: Nc1c(C(=O)Nc2ccc(C(=O)N3CCN(C(=O)[C@H]4[C@@H]5CN(CCCO)C[C@@H]54)CC3)c(Cl)c2)ncn1-c1cn(CC2CCC2)nc1C(F)(F)F ; Cl]

### Step 1: tert-butyl rac-(1S,5R)-6-[4-[4-[[5-amino-1-[1-(cyclobutylmethyl)-3-(trifluoromethyl)pyrazol-4-yl]imidazole-4-carbonyl]amino]-2-chloro-benzoyl]piperazine-1-carbonyl]-3-azabicyclo[3.1.0]hexane-3-carboxylate

To a solution of 5-amino-N-[3-chloro-4-(piperazine-1-carbonyl)phenyl]-1-[1-(cyclobutylmethyl)-3-(trifluoromethyl)pyrazol-4-yl]imidazole-4-carboxamide;hydrochloride (Intermediate 85, 170 mg, 0.29 mmol, 1.0 eq) and rac-(1S,5R)-3-tert-butoxycarbonyl-3-azabicyclo[3.1.0]hexane-6-carboxylic acid (65 mg, 0.29 mmol, 1.0 eq) in DMF (2 mL) was added N,N-diisopropylethylamine (0.15 mL, 0.87 mmol, 3.0 eq) followed by HATU (220 mg, 0.58 mmol, 2.0 eq) at 10° C. The resulting mixture was then stirred at 10° C. for 1 h. The mixture was purified by reverse phase HPLC (H₂O (0.1% FA)-MeCN; 70-80% B) to afford the title compound (210 mg, 0.28 mmol, 95% yield). MS: m/z=760.5 [M+H]+, ESI pos.

### Step 2: 5-amino-N-[3-chloro-4-[4-[rac-(1S,5R)-3-azabicyclo[3.1.0]hexane-6-carbonyl]piperazine-1-carbonyl]phenyl]-1-[1-(cyclobutylmethyl)-3-(trifluoromethyl)pyrazol-4-yl]imidazole-4-carboxamide;hydrochloride

A solution of tert-butyl rac-(1S,5R)-6-[4-[4-[[5-amino-1-[1-(cyclobutylmethyl)-3-(trifluoromethyl)pyrazol-4-yl]imidazole-4-carbonyl]amino]-2-chloro-benzoyl]piperazine-1-carbonyl]-3-azabicyclo[3.1.0]hexane-3-carboxylate (190.0 mg, 0.25 mmol, 1.0 eq) in HCl/dioxane (3.0 mL, 2 M) was stirred at 10° C. for 1 h. The mixture was concentrated under reduced pressure to afford the title compound (160 mg, 0.23 mmol, 97% yield). MS: m/z=660.2 [M+H]+, ESI pos.

### Step 3: 5-amino-N-[3-chloro-4-[4-[rac-(1S,5R)-3-[3-[tert-butyl(dimethyl)silyl]oxypropyl]-3-azabicyclo[3.1.0]hexane-6-carbonyl]piperazine-1-carbonyl]phenyl]-1-[1-(cyclobutylmethyl)-3-(trifluoromethyl)pyrazol-4-yl]imidazole-4-carboxamide

To a solution of 5-amino-N-[3-chloro-4-[4-[rac-(1S,5R)-3-azabicyclo[3.1.0]hexane-6-carbonyl]piperazine-1-carbonyl]phenyl]-1-[1-(cyclobutylmethyl)-3-(trifluoromethyl)pyrazol-4-yl]imidazole-4-carboxamide;hydrochloride (120 mg, 0.17 mmol, 1.0 eq) in MeOH (3 mL) was added 3-[tert-butyl(dimethyl)silyl]oxypropanal (32 mg, 0.17 mmol, 1.0 eq) and sodium acetate (0.07 mL, 0.86 mmol, 5.0 eq). After stirred at 10° C. for 1 h, sodium cyanoborohydride (21 mg, 0.34 mmol, 2.0 eq) was added to the mixture. The resulting mixture was then stirred at 10° C. for another 1 h. The mixture was concentrated under reduced pressure. The residue was purified by reverse phase HPLC (0.1% FA in H₂O-MeCN, 60-80% B) to afford the title compound (70 mg, 0.08 mmol, 49% yield). MS: m/z=832.4 [M+H]+, ESI pos.

### Step 4: 5-amino-N-[3-chloro-4-[4-[rac-(1S,5R)-3-(3-hydroxypropyl)-3-azabicyclo[3.1.0]hexane-6-carbonyl]piperazine-1-carbonyl]phenyl]-1-[1-(cyclobutylmethyl)-3-(trifluoromethyl)pyrazol-4-yl]imidazole-4-carboxamide;hydrochloride

A solution of 5-amino-N-[3-chloro-4-[4-[rac-(1S,5R)-3-[3-[tert-butyl(dimethyl)silyl]oxypropyl]-3-azabicyclo[3.1.0]hexane-6-carbonyl]piperazine-1-carbonyl]phenyl]-1-[1-(cyclobutylmethyl)-3-(trifluoromethyl)pyrazol-4-yl]imidazole-4-carboxamide (60 mg, 0.07 mmol, 1.0 eq) in HCl/dioxane (3.0 mL, 2 M) was stirred at 10° C. for 1 h. The mixture was concentrated under reduced pressure. The residue was purified by prep-HPLC (Instrument: Gilson GX-281+Shimadzu 20AP Pump+SPD-20A Detector, column: Phenomenex luna C18 150×25 mm×10 um, mobile phase: A: H₂O (0.2% FA); B: MeCN; 15-45% B, 10 min) to afford the title compound (8.3 mg, 0.01 mmol, 15% yield). MS: m/z=718.3 [M+H]+, ESI pos.

### Compound 292

### N-[4-[4-[(3aS,6aR)-2-methyl-1,3,3a, 4,6,6a-hexahydropyrrolo[3,4-c]pyrrole-5-carbonyl]piperazine-1-carbonyl]-3-fluorophenyl]-5-amino-1-[2-(difluoromethyl)-3-fluoro-4-(fluoromethoxy)phenyl]imidazole-4-carboxamide;2,2,2-trifluoroacetic acid

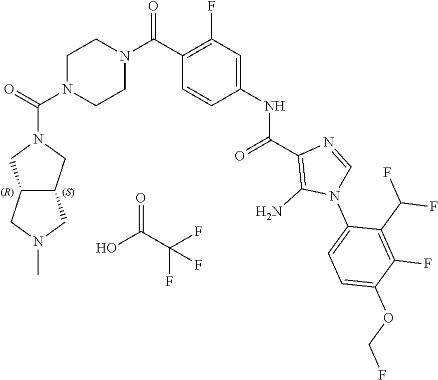

### Step 3: tert-butyl N-[5-[[4-[4-[(3aS,6aR)-2-methyl-1,3,3a, 4,6,6a-hexahydropyrrolo[3,4-c]pyrrole-5-carbonyl]piperazine-1-carbonyl]-3-fluorophenyl]carbamoyl]-3-[2-(difluoromethyl)-3-fluoro-4-(fluoromethoxy)phenyl]imidazol-4-yl]carbamate

To a solution of [4-[(3aS,6aR)-2-methyl-1,3,3a,4,6,6a-hexahydropyrrolo[3,4-c]pyrrole-5-carbonyl]piperazin-1-yl]-(4-amino-2-fluorophenyl) methanone (Intermediate 53, 40 mg, 0.11 mmol, 1.0 eq) ethyl 5-[bis(tert-butoxycarbonyl)amino]-1-[2-(difluoromethyl)-3-fluoro-4-(fluoromethoxy)phenyl]imidazole-4-carboxylate (Intermediate 38, 58 mg, 0.11 mmol, 1.0 eq) in THF (2 mL) was added dropwise lithium bis(trimethylsilyl)amide (0.43 mL, 0.43 mmol, 4.0 eq) at −78° C. under N₂ for 0.5 h. Then the solution was warmed to 20° C. and poured into saturated NH₄Cl aqueous solution (10 mL), then extracted with EtOAc (20 mL). Combined organic phase was washed with brine (20 mL), dried over Na₂SO₄, filtered, and concentrated under reduced pressure. The residue was purified by reverse phase HPLC (H₂O (0.1% FA)-CAN; 20-40% B) to afford the title compound (15 mg, 0.02 mmol, 18% yield). MS: m/z=777.3 [M+H]+, ESI pos.

### Step 4: N-[4-[4-[(3aS,6aR)-2-methyl-1,3,3a,4,6,6a-hexahydropyrrolo[3,4-c]pyrrole-5-carbonyl]piperazine-1-carbonyl]-3-fluorophenyl]-5-amino-1-[2-(difluoromethyl)-3-fluoro-4-(fluoromethoxy)phenyl]imidazole-4-carboxamide;2,2,2-trifluoroacetic acid

A solution of tert-butyl N-[5-[[4-[4-[(3aS,6aR)-2-methyl-1,3,3a,4,6,6a-hexahydropyrrolo[3,4-c]pyrrole-5-carbonyl]piperazine-1-carbonyl]-3-fluorophenyl]carbamoyl]-3-[2-(difluoromethyl)-3-fluoro-4-(fluoromethoxy)phenyl]imidazol-4-yl]carbamate (15 mg, 0.02 mmol, 1.0 eq) in HCl/dioxane (0.5 mL, 2 M) was stirred at 20° C. for 0.5 h. The mixture was concentrated under reduced pressure. The residue was purified by prep-HPLC (column: Phenomenex luna C18 150×25 mm×10 um; condition: H₂O (TFA)-MeCN; 12-42% B) to afford the title compound (8.6 mg, 0.01 mmol, 65% yield). MS: m/z=677.2 [M+H]+, ESI pos.

The following compounds were prepared in analogy to the procedure above

| Compound | Reagents | MS [M + H]+ |
|---|---|---|
| 293 | Intermediate 38, | 677.4 |
| | Intermediate 52 | |
| 294 | Intermediate 46, | 679.2 |
| | Intermediate 54 | |
| 295 | Intermediate 38, | 676.4 |
| | Intermediate 57 | |

### Compound 296

### 5-amino-N-[3-chloro-4-[4-[(1R,4R)-5-methyl-2,5-diazabicyclo[2.2.1]heptane-2-carbonyl]piperazine-1-carbonyl]phenyl]-1-[2-(difluoromethyl)-3-fluoro-4-(fluoromethoxy)phenyl]imidazole-4-carboxamide;2,2,2-trifluoroacetic acid

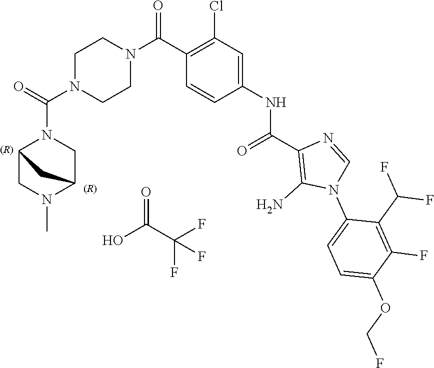

### Step 1: tert-butyl N-[5-[[3-chloro-4-[4-[(1R,4R)-5-methyl-2,5-diazabicyclo[2.2.1]heptane-2-carbonyl]piperazine-1-carbonyl]phenyl]carbamoyl]-3-[2-(difluoromethyl)-3-fluoro-4-(fluoromethoxy)phenyl]imidazol-4-yl]carbamate

To a mixture of 5-(tert-butoxycarbonylamino)-1-[2-(difluoromethyl)-3-fluoro-4-(fluoromethoxy)phenyl]imidazole-4-carboxylic acid (Intermediate 46, 111 mg, 0.27 mmol, 1.0 eq), (4-amino-2-chlorophenyl)-[4-[(1R,4R)-5-methyl-2,5-diazabicyclo[2.2.1]heptane-2-carbonyl]piperazin-1-yl]methanone;hydrochloride (Intermediate 55, 110 mg, 0.27 mmol, 1.0 eq) and 1-methylimidazole (0.08 mL, 1.06 mmol, 4.0 eq) in CH₂Cl₂ (5 mL) was added T4P (573 mg, 0.8 mmol, 3.0 eq). The mixture was stirred at 10° C. for 1 h. The mixture was purified by reverse phase HPLC (H₂O (0.1% FA)-MeCN]; 55-60% B) to afford the title compound (30 mg, 0.04 mmol, 13% yield) as yellow oil. MS: m/z=777.2 [M−H]+, ESI pos.

### Step 2: 5-amino-N-[3-chloro-4-[4-[(1R,4R)-5-methyl-2,5-diazabicyclo[2.2.1]heptane-2-carbonyl]piperazine-1-carbonyl]phenyl]-1-[2-(difluoromethyl)-3-fluoro-4-(fluoromethoxy)phenyl]imidazole-4-carboxamide;2,2,2-trifluoroacetic acid

A mixture of tert-butyl N-[5-[[3-chloro-4-[4-[(1R,4R)-5-methyl-2,5-diazabicyclo[2.2.1]heptane-2-carbonyl]piperazine-1-carbonyl]phenyl]carbamoyl]-3-[2-(difluoromethyl)-3-fluoro-4-(fluoromethoxy)phenyl]imidazol-4-yl]carbamate (40 mg, 0.05 mmol, 1.0 eq) in HCl/dioxane (1.5 mL, 2 M) was stirred at 10° C. for 1 h. The reaction mixture was concentrated under reduced pressure. The residue was purified by prep-HPLC (column: Waters Xbridge 150×25 mm×5 um; mobile phase: [H₂O (TFA)-MeCN]; 15-45% B, 18 min) to afford the title compound (5.6 mg, 0.01 mmol, 13% yield). MS: m/z=679.4 [M+H]+, ESI pos.

### Compound 297

### 5-amino-N-[3-chloro-4-[4-[2-(4-piperidyloxy) acetyl]piperazine-1-carbonyl]phenyl]-1-[2,3-difluoro-4-(fluoromethoxy)phenyl]imidazole-4-carboxamide;formic acid

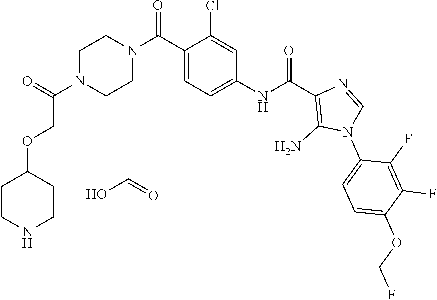 [SMILES: Nc1c(C(=O)Nc2ccc(C(=O)N3CCN(C(=O)COC4CCNCC4)CC3)c(Cl)c2)ncn1-c1ccc(OCF)c(F)c1F ; O=CO]

### Step 1: tert-butyl 4-[2-[4-[4-[[5-amino-1-[2,3-difluoro-4-(fluoromethoxy)phenyl]imidazole-4-carbonyl]amino]-2-chloro-benzoyl]piperazin-1-yl]-2-oxo-ethoxy]piperidine-1-carboxylate

To a solution of 5-amino-N-[3-chloro-4-(piperazine-1-carbonyl)phenyl]-1-[2,3-difluoro-4-(fluoromethoxy)phenyl]imidazole-4-carboxamide;hydrochloride (42 mg, 0.08 mmol, 1.0 eq) and 2-[(1-tert-butoxycarbonyl-4-piperidyl)oxy]acetic acid (24 mg, 0.09 mmol, 1.2 eq) in DMF (1 mL) was added N,N-diisopropylethylamine (0.04 mL, 0.24 mmol, 3.0 eq) and HATU (29 mg, 0.08 mmol, 1.0 eq), the mixture was stirred at 20° C. for 2 h. The mixture was purified by reverse phase HPLC (H₂O (0.1% FA)-MeCN, 20-25% B) to afford the title compound (45 mg, 0.06 mmol, 74% yield). MS: m/z=750.3 [M+H]+, ESI pos.

### Step 2: 5-amino-N-[3-chloro-4-[4-[2-(4-piperidyloxy) acetyl]piperazine-1-carbonyl]phenyl]-1-[2,3-difluoro-4-(fluoromethoxy)phenyl]imidazole-4-carboxamide; formic acid

A stirred solution of tert-butyl 4-[2-[4-[4-[[5-amino-1-[2,3-difluoro-4-(fluoromethoxy)phenyl]imidazole-4-carbonyl]amino]-2-chloro-benzoyl]piperazin-1-yl]-2-oxo-ethoxy]piperidine-1-carboxylate (40 mg, 0.05 mmol, 1.0 eq) in HCl/dioxane (3.24 mL, 2 M) was stirred at 20° C. for 0.5 h. The mixture was purified by prep-HPLC (Phenomenex luna C18 150×25 mm×10 um; mobile phase: [H₂O (HCl)-MeCN]; 11-41% B, 9 min) to afford the title compound (15.2 mg, 0.02 mmol, 40% yield). MS: m/z=650.1 [M+H]+, ESI pos.

The following compounds were prepared in analogy to the procedure above

| | | MS |
|---|---|---|
| Compound | Reagents | [M + H]+ |
| 298 | Intermediate 67, | 622.2 |
| | (S)-1-BOC-pyrrolidine-3-carboxylic acid | |
| 299 | Intermediate 68, | 652.1 |
| | 2-[(3R)-1-tert-butoxycarbonylpyrrolidin-3-yl] | |
| | acetic acid | |
| 300 | Intermediate 68, | 650.2 |
| | 2-tert-butoxycarbonyl-2-azabicyclo[2.1.1] | |
| | hexane-4-carboxylic acid | |
| 301 | Intermediate 68, | 654.2 |
| | (3S,4R)-1-tert-butoxycarbonyl-4-hydroxy- | |
| | pyrrolidine-3-carboxylic acid | |
| 302 | Intermediate 68, | 654.2 |
| | (3S,4S)-1-tert-butoxycarbonyl-4-hydroxy- | |
| | pyrrolidine-3-carboxylic acid | |
| 303 | Intermediate 68, | 666.2 |
| | 1-BOC-4-piperidylacetic aci | |
| 304 | Intermediate 68, | 664.2 |
| | 2-tert-butoxycarbonyl-2-azabicyclo[3.1.1 ] | |
| | heptane-5-carboxylic acid | |
| 305 | Intermediate 68, | 664.4 |
| | 3-tert-butoxycarbonyl-3-azabicyclo[3.1.1] | |
| | heptane-1-carboxylic acid | |
| 306 | Intermediate 68, | 644.3 |
| | (1S,5R)-3-tert-butoxycarbonyl-3- | |
| | azabicyclo[3.1.1]heptane-6-carboxylic | |
| | acid | |
| 307 | Intermediate 68, | 664.5 |
| | (1S,4R,5R)-2-tert-butoxycarbonyl-2- | |
| | azabicyclo[2.2.1]heptane-5-carboxylic | |
| | acid | |
| 308 | Intermediate 68, | 664.2 |
| | 2-((1R,5S,6s)-3-(tert-butoxycarbonyl)-3- | |
| | azabicyclo[3.1.0]hexan-6-yl)acetic | |
| | acid | |
| 309 | Intermediate 68, | 668.2 |
| | 2-[(3S)-1-tert-butoxycarbonylpyrrolidin-3-yl] | |
| | oxyacetic acid | |
| 310 | Intermediate 68, | 668.1 |
| | 2-[(3R)-1-tert-butoxycarbonylpyrrolidin-3-yl] | |
| | oxyacetic acid | |
| 311 | Intermediate 69, | 682.2 |
| | 1-BOC-4-fluoro-4-piperidinecarboxylic acid | |
| 312 | Intermediate 70, | 620.3 |
| | 2-[(3S)-1-tert-butoxycarbonylpyrrolidin-3-yl] | |
| | acetic acid | |
| 313 | Intermediate 70, | 648.2 |
| | 2-tert-butoxycarbonyl-4-(hydroxymethyl)-2- | |
| | azabicyclo[2.1.1 ]hexane-1-carboxylic acid | |
| 314 | Intermediate 70, | 648.3 |
| | 2-tert-butoxycarbonyl-1-(hydroxymethyl)-2- | |
| | azabicyclo[2.1.1]hexane-4-carboxylic acid | |
| 315 | Intermediate 70, | 632.2 |
| | (1S,4R,5R)-2-tert-butoxycarbonyl-2- | |
| | azabicyclo[2.2.1 ]heptane-5-carboxylic | |
| | acid | |
| 316 | Intermediate 70, | 620.1 |
| | BOC-L-BETA-HOMOPROLINE | |
| 317 | Intermediate 70, | 637.2 |
| | (3S)-3,6-bis(tert-butoxycarbonylamino) | |
| | hexanoic acid | |
| 318 | Intermediate 74 Intermediate 63 | 654.3 |
| 319 | Intermediate 71, | 676.3 |
| | (2S,4R)-1-tert-butoxycarbonyl-4- | |
| | (hydroxymethyl)pyrrolidine-2-carboxylic | |
| | acid | |
| 320 | Intermediate 71, | 676.3 |
| | (2S,4S)-1-tert-butoxycarbonyl-4- | |
| | (hydroxymethyl)pyrrolidine-2-carboxylic | |
| | acid | |

and

| Inter- | | | | |
|---|---|---|---|---|
| mediate | | | | MS |
| Num- | | | | [M + |
| ber | Structure | Name | Reagents | H]+ |
| 135 | 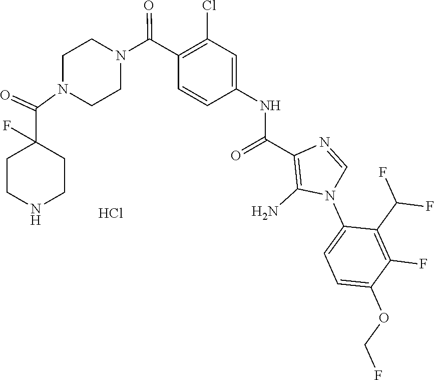 [SMILES: Nc1c(C(=O)Nc2ccc(C(=O)N3CCN(C(=O)C4(F)CCNCC4)CC3)c(Cl)c2)ncn1-c1ccc(OCF)c(F)c1C(F)F ; Cl] | 5-amino-N- [3-chloro-4- [4-(4-fluoro- piperidine-4- carbonyl) piperazine-1- carbonyl] phenyl]-1-[2- (difluoro- methyl)-3- fluoro-4- (fluoro- methoxy) phenyl] imidazole-4- carboxamide; hydrochloride | Inter- mediate 68, Inter- mediate 58 | 670.2 |
| 136 | 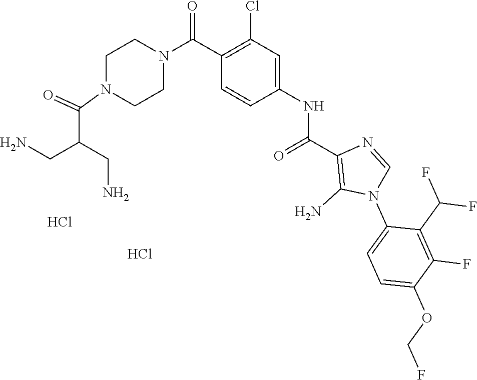 [SMILES: NCC(CN)C(=O)N1CCN(C(=O)c2ccc(NC(=O)c3ncn(-c4ccc(OCF)c(F)c4C(F)F)c3N)cc2Cl)CC1 ; Cl ; Cl] | 5-amino- N-[4-[4-[3- amino-2- (aminomethyl) propanoyl] piperazine-1- carbonyl]- 3-chloro- phenyl]-1-[2- (difluoro- methyl)-3- fluoro-4- (fluoro- methoxy) phenyl] imidazole-4- carboxamide; dihydrochloride | Inter- mediate 68, 3-(tert- butoxy- carbonyl- amino)- 2- [(tert- butoxy- carbonyl- amino) methyl] propanoic acid | 641.2 |
| 137 | 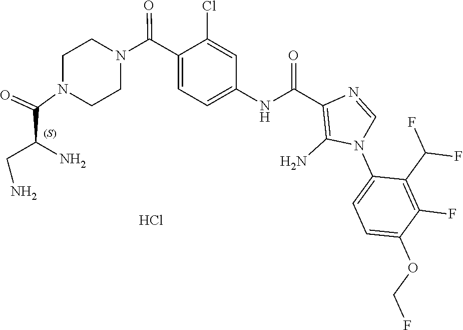 | 5-amino-N- [3-chloro-4- [4-[(2S)-2,3- diamino- propanoyl] piperazine-1- carbonyl] phenyl]-1-[2- (difluoro- methyl)-3- fluoro-4- (fluoromethoxy) phenyl] imidazole-4- carboxamide; hydrochloride | Inter- mediate 68, (2S)-2,3- bis(tert- butoxy- carbonyl- amino) propanoic acid | 627.1 |
| 138 | 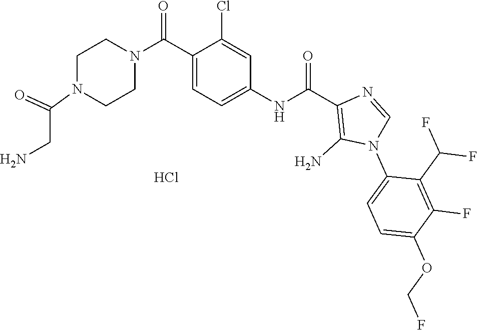 [SMILES: NCC(=O)N1CCN(C(=O)c2ccc(NC(=O)c3ncn(-c4ccc(OCF)c(F)c4C(F)F)c3N)cc2Cl)CC1 ; Cl] | 5-amino-N- [4-[4-(2- aminoacetyl) piperazine- 1-carbonyl]-3- chloro- phenyl]-1-[2- (difluoro- methyl)-3- fluoro-4- (fluoromethoxy) phenyl] imidazole-4- carboxamide; hydrochloride | Inter- mediate 68, BOC- glycine | 598.1 |
| 139 | 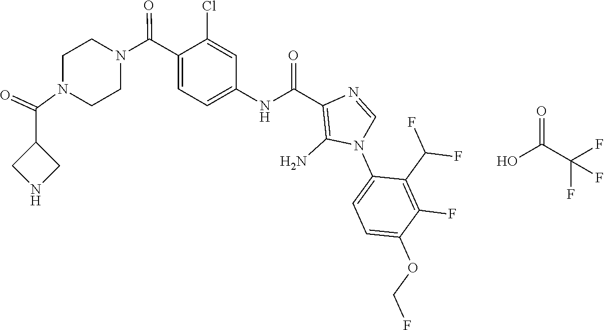 [SMILES: Nc1c(C(=O)Nc2ccc(C(=O)N3CCN(C(=O)C4CNC4)CC3)c(Cl)c2)ncn1-c1ccc(OCF)c(F)c1C(F)F ; O=C(O)C(F)(F)F] | 5-amino-N-[4-[4- (azetidine-3- carbonyl) piperazine-1- carbonyl]-3- chlorophenyl]- 1-[2- (difluoromethyl)- 3- fluoro-4- (fluoromethoxy) phenyl] imidazole-4- carboxamide; 2,2,2- trifluoroacetic acid | Inter- mediate 68, 1-BOC- azetidine- 3- carboxylic acid | 624.1 |
| 140 | 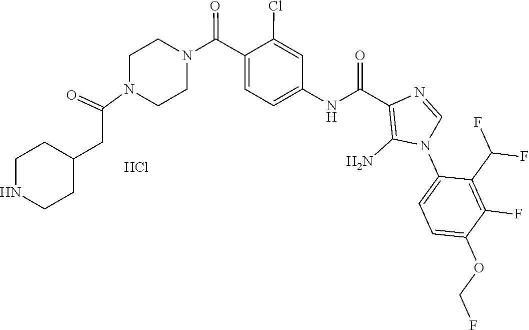 [SMILES: Nc1c(C(=O)Nc2ccc(C(=O)N3CCN(C(=O)CC4CCNCC4)CC3)c(Cl)c2)ncn1-c1ccc(OCF)c(F)c1C(F)F ; Cl] | 5-amino-N- [3-chloro-4- [4-[2-(4- piperidyl)acetyl] piperazine- 1-carbonyl] phenyl]- 1-[2-(difluoro- methyl)-3- fluoro-4- (fluoromethoxy) phenyl] imidazole-4- carboxamide; hydrochlorid | Inter- mediate 68, 1- BOC-4- piperidyl- acetic acid | 666.3 |
| 141 | 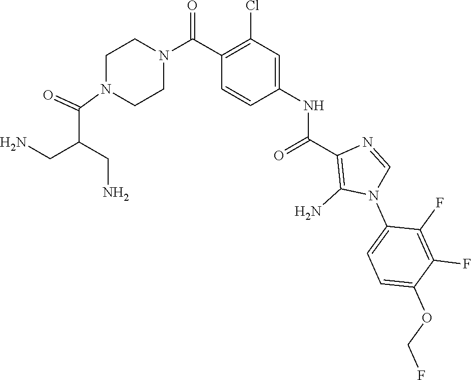 [SMILES: NCC(CN)C(=O)N1CCN(C(=O)c2ccc(NC(=O)c3ncn(-c4ccc(OCF)c(F)c4F)c3N)cc2Cl)CC1] | 5-amino-N- [4-[4-[3- amino-2- (aminomethyl) propanoyl] piperazine-1- carbonyl]- 3-chlorophenyl]- 1-[2,3- difluoro-4- (fluoromethoxy) phenyl] imidazole-4- carboxamide | Inter- mediate 70, 3-(tert- butoxy- carbonyl- amino)-2- [(tert- butoxy- carbonyl- amino) methyl] propanoic acid | 609.3 |
| 142 | 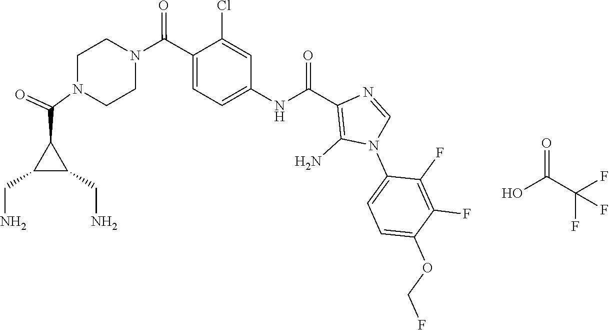 [SMILES: NC[C@@H]1[C@H](CN)[C@H]1C(=O)N1CCN(C(=O)c2ccc(NC(=O)c3ncn(-c4ccc(OCF)c(F)c4F)c3N)cc2Cl)CC1 ; O=C(O)C(F)(F)F] | 5-amino- N-[4-[4- [(2R,3S)-2,3- bis(amino- methyl) cyclopropane- carbonyl] piperazine-1- carbonyl]-3- chlorophenyl]- 1-[2,3- difluoro-4- (fluoromethoxy) phenyl] imidazole-4- carboxamide; 2,2,2- trifluoroacetic acid | Inter- mediate 70, Inter- mediate 121 | 635.4 |

### Compound 321

### 5-amino-N-[4-[4-(1-carbamimidoyl-4-fluoro-piperidine-4-carbonyl)piperazine-1-carbonyl]-3-chlorophenyl]-1-[2-(difluoromethyl)-3-fluoro-4-(fluoromethoxy)phenyl]imidazole-4-carboxamide; formic acid

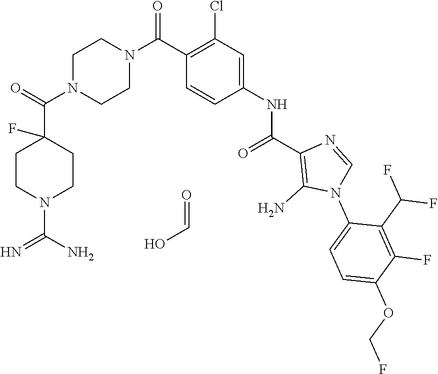 [SMILES: N=C(N)N1CCC(F)(C(=O)N2CCN(C(=O)c3ccc(NC(=O)c4ncn(-c5ccc(OCF)c(F)c5C(F)F)c4N)cc3Cl)CC2)CC1 ; O=CO]

### Step 1: 5-amino-N-[4-[4-(1-carbamimidoyl-4-fluoro-piperidine-4-carbonyl)piperazine-1-carbonyl]-3-chlorophenyl]-1-[2-(difluoromethyl)-3-fluoro-4-(fluoromethoxy)phenyl]imidazole-4-carboxamide; formic acid

To a mixture of 1H-pyrazole-1-carboxamidine hydrochloride (31 mg, 0.21 mmol, 5.0 eq) and 5-amino-N-[3-chloro-4-[4-(4-fluoropiperidine-4-carbonyl)piperazine-1-carbonyl]phenyl]-1-[2-(difluoromethyl)-3-fluoro-4-(fluoromethoxy)phenyl]imidazole-4-carboxamide;hydrochloride (Intermediate 135, 30 mg, 0.04 mmol, 1.0 eq) in EtOH (1 mL) was added triethylamine (0.02 mL, 0.17 mmol, 4.0 eq) at 15° C., the mixture was stirred for 16 h. The reaction mixture was concentrated under reduced pressure. The residue was purified by prep-HPLC (column: Waters Xbridge 150×25 mm×5 um; mobile phase: [H₂O (FA)-MeCN]; 10-40% B, 10 min) to afford the title compound (15 mg, 0.02 mmol, 45% yield). MS: m/z=712.1 [M+H]+, ESI pos.

The following compound was prepared in analogy to the procedure above

| | | MS |
|---|---|---|
| Compound | Reagents | [M + H]+ |
| 322 | ethyl acetimidate hydrochloride | 711.2 |

### Compound 323

### 5-amino-N-[3-chloro-4-[4-(2-iminohexahydropyrimidine-5-carbonyl)piperazine-1-carbonyl]phenyl]-1-[2,3-difluoro-4-(fluoromethoxy)phenyl]imidazole-4-carboxamide;formic acid

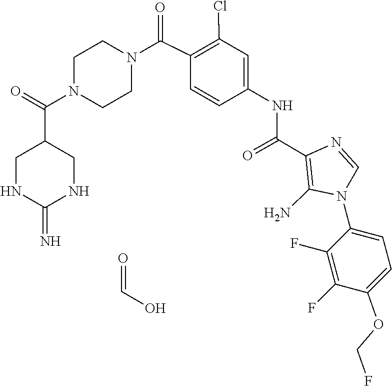 [SMILES: N=C1NCC(C(=O)N2CCN(C(=O)c3ccc(NC(=O)c4ncn(-c5ccc(OCF)c(F)c5F)c4N)cc3Cl)CC2)CN1 ; O=CO]

To a solution of 5-amino-N-[4-[4-[3-amino-2-(aminomethyl) propanoyl]piperazine-1-carbonyl]-3-chlorophenyl]-1-[2,3-difluoro-4-(fluoromethoxy)phenyl]imidazole-4-carboxamide (Intermediate 141, 50 mg, 0.08 mmol, 1.0 eq) in THF (0.5 mL) was added di(1H-imidazol-1-yl) methanimine (26 mg, 0.16 mmol, 2.0 eq) and imidazole (16 mg, 0.25 mmol, 3.0 eq). Then the mixture was then stirred at 35° C. for 12 h. The reaction mixture was concentrated under reduced pressure. The residue was purified by prep-HPLC (Phenomenex luna C18 150×25 mm×10 um; mobile phase: [H₂O (0.1% FA, V/V)-MeCN]; 13-43% B, 11 min) to afford the title compound (17.8 mg, 0.03 mmol, 30% yield). MS: m/z=634.1 [M+H]+, ESI pos.

The following compounds were prepared in analogy to the procedure above

| Compound | Reagents | MS [M + H]+ |
|---|---|---|
| 324 | Intermediate 136 | 666.2 |
| 325 | Intermediate 137 | 652.1 |
| 326 | Intermediate 142 | 660.3 |

### Compound 327

### 5-amino-N-[3-chloro-4-[4-[2-(4,5-dihydro-1H-imidazol-2-ylamino) acetyl]piperazine-1-carbonyl]phenyl]-1-[2-(difluoromethyl)-3-fluoro-4-(fluoromethoxy)phenyl]imidazole-4-carboxamide; formic acid

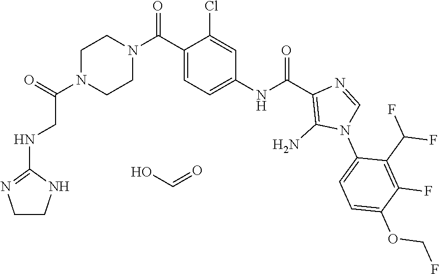 [SMILES: Nc1c(C(=O)Nc2ccc(C(=O)N3CCN(C(=O)CNC4=NCCN4)CC3)c(Cl)c2)ncn1-c1ccc(OCF)c(F)c1C(F)F ; O=CO]

To a solution of 5-amino-N-[4-[4-(2-aminoacetyl)piperazine-1-carbonyl]-3-chlorophenyl]-1-[2-(difluoromethyl)-3-fluoro-4-(fluoromethoxy)phenyl]imidazole-4-carboxamide;hydrochloride (Intermediate 138, 60 mg, 0.09 mmol, 1.0 eq) and 2-methylthio-2-imidazoline hydriodide (69 mg, 0.28 mmol, 3.0 eq) in DMF (1 mL) was added triethylamine (47 mg, 0.47 mmol, 5.0 eq). Then the mixture was then stirred at 80° C. for 2 h. The reaction mixture was purified by Prep HPLC (column: Unisil 3-100 C18 Ultra 150×50 mm×3 um; mobile phase: [H₂O (0.1% FA, V/V)-MeCN]; 10-40% B, 2 min) to afford the title compound (5.7 mg, 0.01 mmol, 8% yield). MS: m/z=666.1 [M+H]+, ESI pos.

The following compounds were prepared in analogy to the procedure above

| Compound | Reagents | MS [M + H]+ |
|---|---|---|
| 328 | Intermediate 139 | 692.3 |
| 329 | Intermediate 143 | 706.4 |
| 330 | Intermediate 138, | 708.2 |
| | Intermediate 120 | |

### Compound 331

### 5-amino-N-[3-chloro-4-[4-[(6S)-2-oxa-7-azaspiro[3.4]octane-6-carbonyl]piperazine-1-carbonyl]phenyl]-1-[2,3-difluoro-4-(fluoromethoxy)phenyl]imidazole-4-carboxamide

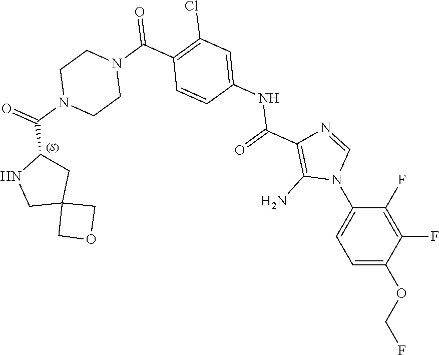

### Step 1: 9H-fluoren-9-ylmethyl (6S)-6-[4-[4-[[5-amino-1-[2,3-difluoro-4-(fluoromethoxy)phenyl]imidazole-4-carbonyl]amino]-2-chloro-benzoyl]piperazine-1-carbonyl]-2-oxa-7-azaspiro[3.4]octane-7-carboxylate

To a solution of 5-amino-N-[3-chloro-4-(piperazine-1-carbonyl)phenyl]-1-[2,3-difluoro-4-(fluoromethoxy)phenyl]imidazole-4-carboxamide (Intermediate 70, 120 mg, 0.24 mmol, 1.0 eq), 7-(9H-fluoren-9-ylmethoxycarbonyl)-2-oxa-7-azaspiro[3.4]octane-6-carboxylic acid (153 mg, 0.28 mmol, 1.2 eq) and N,N-diisopropylethylamine (0.16 mL, 0.94 mmol, 4.0 eq) in DMF (0.5 mL) was added O-(7-azabenzotriazol-1-yl)-N,N,N′,N′-tetramethyluronium hexafluorophosphate (134 mg, 0.35 mmol, 1.5 eq) at 25° C., the mixture was stirred at 25° C. for 1 h. The reaction mixture was purified by reverse phase flash (0.1% FA, H₂O-MeCN, 60-70% B) to give a crude product, which was purified by prep-SFC (column: DAICEL CHIRALPAK IC, 250 mm×30 mm, 10 um; mobile phase: [CO₂-MeCN/EtOH (0.1% NH₃·H₂O)]; 75-75% B, 8.8 min) to afford the title compound (50 mg, 0.06 mmol, 45.45% yield), and 9H-fluoren-9-ylmethyl (6R)-6-[4-[4-[[5-amino-1-[2,3-difluoro-4-(fluoromethoxy)phenyl]imidazole-4-carbonyl]amino]-2-chloro-benzoyl]piperazine-1-carbonyl]-2-oxa-7-azaspiro[3.4]octane-7-carboxylate (45.0 mg, 0.05 mmol, 38% yield). MS: m/z=870.3 [M+H]+, ESI pos.

### Step 2: 5-amino-N-[3-chloro-4-[4-[(6S)-2-oxa-7-azaspiro[3.4]octane-6-carbonyl]piperazine-1-carbonyl]phenyl]-1-[2,3-difluoro-4-(fluoromethoxy)phenyl]imidazole-4-carboxamide

To a mixture of 9H-fluoren-9-ylmethyl (6S)-6-[4-[4-[[5-amino-1-[2,3-difluoro-4-(fluoromethoxy)phenyl]imidazole-4-carbonyl]amino]-2-chlorobenzoyl]piperazine-1-carbonyl]-2-oxa-7-azaspiro[3.4]octane-7-carboxylate (50 mg, 0.06 mmol, 1.0 eq) in DMF (0.5 mL) was added piperidine (24 mg, 0.29 mmol, 5.0 eq). The mixture was then stirred at 20° C. for 1 h. The reaction mixture was purified by reverse phase HPLC (0.1% NH₃·H₂O, H₂O-MeCN, 30-40% B) to afford the title compound (14.2 mg, 0.02 mmol, 38% yield). MS: m/z=648.3 [M+H]+, ESI pos.

The following compounds were prepared in analogy to the procedure above

| | | MS |
|---|---|---|
| Compound | Reagents | [M + H]+ |
| 332 | Intermediate 70, | 648.2 |
| | 7-(9H-fluoren-9-ylmethoxycarbonyl)-2-oxa-7- | |
| | azaspiro[3.4]octane-6-carboxylic acid | |
| 333 | Intermediate 68, | 680.2 |
| | 7-(9H-fluoren-9-ylmethoxycarbonyl)-2-oxa-7- | |
| | azaspiro[3.4]octane-6-carboxylic acid | |
| 334 | Intermediate 70, | 648.1 |
| | 5-(9H-fluoren-9-ylmethoxycarbonyl)-2-oxa-5- | |
| | azaspiro[3.4]octane-7-carboxylic acid | |
| 335 | Intermediate 70, | 648.1 |
| | 5-(9H-fluoren-9-ylmethoxycarbonyl)-2-oxa-5- | |
| | azaspiro[3.4]octane-7-carboxylic acid | |
| 336 | Intermediate 68, | 680.2 |
| | (7S)-6-(tert-butoxycarbonyl)-1-oxa-6- | |
| | azaspiro[3.4]octane-7-carboxylic acid [CAS: | |
| | 2766205-01-8]/with HFIP | |
| 337 | Intermediate 68, | 678.2 |
| | Intermediate 64 | |
| 338 | Intermediate 68, | 664.4 |
| | (1R,3r,5S)-6-(((9H-fluoren-9- | |
| | yl)methoxy)carbonyl)-6- | |
| | azabicyclo[3.1. 1 ]heptane-3-carboxylic acid | |
| | [CAS: 2869870-80-2] | |

and

| Intermediate | | | | MS |
|---|---|---|---|---|
| number | Structure | Name | Reagents | [M + H]+ |
| 143 | 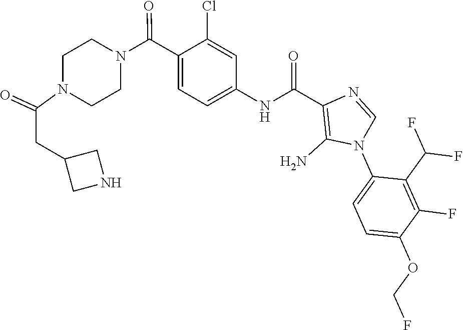 [SMILES: Nc1c(C(=O)Nc2ccc(C(=O)N3CCN(C(=O)CC4CNC4)CC3)c(Cl)c2)ncn1-c1ccc(OCF)c(F)c1C(F)F] | 5-amino-N-[4-[4-[2- (azetidin-3- yl)acetyl]piperazine-1- carbonyl]-3- chlorophenyl]-1-[2- (difluoromethyl)-3- fluoro-4- (fluoromethoxy)phenyl] imidazole-4- carboxamide | Intermediate 68, 2-[1-(9H- fluoren-9- ylmethoxy- carbonyl) azetidin- 3-yl]acetic acid | 638.2 |

### Compound 339

### 5-amino-N-[3-chloro-4-[4-[(1S,4R,5R)-2-methyl-2-azabicyclo[2.2.1]heptane-5-carbonyl]piperazine-1-carbonyl]phenyl]-1-[2,3-difluoro-4-(fluoromethoxy)phenyl]imidazole-4-carboxamide;formic acid

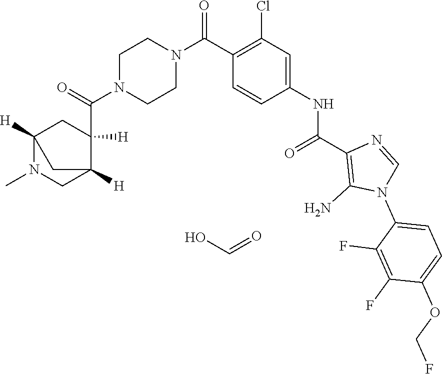 [SMILES: CN1C[C@@H]2C[C@H]1C[C@H]2C(=O)N1CCN(C(=O)c2ccc(NC(=O)c3ncn(-c4ccc(OCF)c(F)c4F)c3N)cc2Cl)CC1 ; O=CO]

To a solution of 5-amino-N-[4-[4-[(1S,4R,5R)-2-azabicyclo[2.2.1]heptane-5-carbonyl]piperazine-1-carbonyl]-3-chlorophenyl]-1-[2,3-difluoro-4-(fluoromethoxy)phenyl]imidazole-4-carboxamide (60 mg, 0.09 mmol, 1.0 eq) in DCE (0.5 mL) was added HCHO (11 mg, 0.14 mmol, 1.5 eq) followed by NaBH(OAc)₃ (40 mg, 0.19 mmol, 2.0 eq). The mixture was then stirred at 20° C. for 1 h. The reaction mixture was concentrated under reduced pressure. The residue was purified by prep-HPLC (column: Phenomenex luna C18 150×25 mm×10 um; mobile phase: [H₂O (0.1% FA, V/V)-MeCN]; 15-45% B, 11 min) to afford the title compound (20 mg, 0.03 mmol, 30% yield). MS: m/z=646.2 [M+H]+, ESI pos.

The following compounds were prepared in analogy to the procedure above

| Compound | Reagents | MS [M + H]+ |
|---|---|---|
| 340 | Compound 306 | 678.4 |
| 341 | Compound 337 | 692.2 |
| 342 | Compound 338 | 678.2 |

### Compound 343

### 5-amino-N-[3-chloro-4-[4-[2-[1-(2-hydroxyethyl)-4-piperidyl]acetyl]piperazine-1-carbonyl]phenyl]-1-[2-(difluoromethyl)-3-fluoro-4-(fluoromethoxy)phenyl]imidazole-4-carboxamide;formic acid

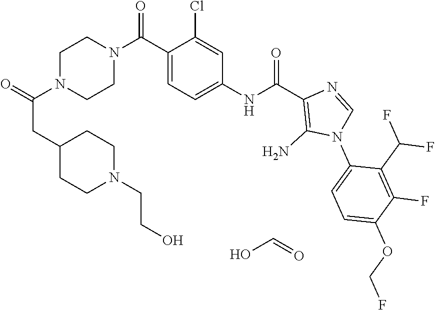 [SMILES: Nc1c(C(=O)Nc2ccc(C(=O)N3CCN(C(=O)CC4CCN(CCO)CC4)CC3)c(Cl)c2)ncn1-c1ccc(OCF)c(F)c1C(F)F ; O=CO]

To a solution of 5-amino-N-[3-chloro-4-[4-[2-(4-piperidyl) acetyl]piperazine-1-carbonyl]phenyl]-1-[2-(difluoromethyl)-3-fluoro-4-(fluoromethoxy)phenyl]imidazole-4-carboxamide;hydrochloride (Intermediate 140, 40 mg, 0.06 mmol, 1.0 eq) and ethylene bromohydrin (4 μL, 0.06 mmol, 1.0 eq) in MeCN (0.5 mL) was added triethylamine (16 μL, 0.11 mmol, 2.0 eq). The mixture was then stirred at 20° C. for 16 h. The mixture was concentrated under reduced pressure. The residue was purified by prep-HPLC (column: Phenomenex luna C18 150×25 mm×10 um; condition: H₂O (FA)-MeCN; 8-38% B) to give the title compound (5.6 mg, 0.01 mmol, 12% yield). MS: m/z=670.2 [M+H]+, ESI pos.

### Compound 344

### [(3S)-pyrrolidin-3-yl] 4-[4-[[5-amino-1-[2-(difluoromethyl)-3-fluoro-4-(fluoromethoxy)phenyl]imidazole-4-carbonyl]amino]-2-chloro-benzoyl]piperazine-1-carboxylate; formic acid

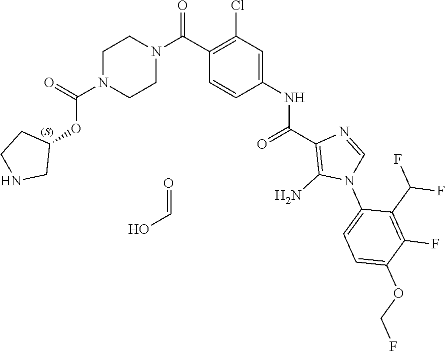

### Step 1: tert-butyl(3S)-3-(4-nitrophenoxy)carbonyloxypyrrolidine-1-carboxylate

To a solution of(S)-1-N-BOC-3-hydroxypyrrolidine (500 mg, 2.67 mmol, 1.0 eq) in CH₂Cl₂ (5 mL) was added DMAP (32 mg, 0.27 mmol, 0.1 eq) and 4-nitrophenyl chloroformate (645 mg, 3.2 mmol, 1.2 eq). The mixture was then stirred at 20° C. for 4 h. The reaction mixture was diluted with H₂O (10 mL), extracted with EtOAc (3×20 mL). The combined organic phase was washed with brine (30 mL), dried over Na₂SO₄, filtered and the filtrate was concentrated under reduced pressure. The residue was purified by silica gel column chromatography (PE:EtOAc=1:0 to 5:1) to afford the title compound (170 mg, 0.48 mmol, 16% yield).

### Step 2: [(3S)-1-tert-butoxycarbonylpyrrolidin-3-yl] 4-[4-[[5-amino-1-[2-(difluoromethyl)-3-fluoro-4-(fluoromethoxy)phenyl]imidazole-4-carbonyl]amino]-2-chloro-benzoyl]piperazine-1-carboxylate

To a solution of tert-butyl(3S)-3-(4-nitrophenoxy)carbonyloxypyrrolidine-1-carboxylate (40 mg, 0.11 mmol, 1.0 eq) and 5-amino-N-[3-chloro-4-(piperazine-1-carbonyl)phenyl]-1-[2-(difluoromethyl)-3-fluoro-4-(fluoromethoxy)phenyl]imidazole-4-carboxamide;2,2,2-trifluoroacetic acid (Intermediate 68, 81 mg, 0.12 mmol, 1.1 eq) in DMF (1 mL) was added DIEA (0.06 mL, 0.34 mmol, 3.0 eq) at 0° C. The mixture was then stirred at 20° C. for 1 h. The reaction mixture was poured into H₂O (2 mL) at 0° C. and filtered to afford the title compound (50 mg, 0.07 mmol, 58% yield). MS: m/z=754.3 [M+H]+, ESI pos.

### Step 3: [(3S)-pyrrolidin-3-yl] 4-[4-[[5-amino-1-[2-(difluoromethyl)-3-fluoro-4-(fluoromethoxy)phenyl]imidazole-4-carbonyl]amino]-2-chloro-benzoyl]piperazine-1-carboxylate; formic acid

A solution of [(3R)-1-tert-butoxycarbonylpyrrolidin-3-yl] 4-[4-[[5-amino-1-[2-(difluoromethyl)-3-fluoro-4-(fluoromethoxy)phenyl]imidazole-4-carbonyl]amino]-2-chloro-benzoyl]piperazine-1-carboxylate (50 mg, 0.07 mmol, 1.0 eq) in HCl/dioxane (1.0 mL, 2 M) was stirred at 20° C. for 2 h. The reaction mixture was concentrated under reduced pressure. The residue was purified by reverse phase flash (CombiFlash mobile phase: [H₂O (0.1% FA v/v)-MeCN]; 30-45% B) to afford the title compound (40 mg, 0.06 mmol, 87% yield). MS: m/z=654.3 [M+H]+, ESI pos.

### Compound 345

### 5-amino-N-[3-chloro-4-[4-[2-[1-[2-hydroxy-1-(hydroxymethyl)ethyl]-4-piperidyl]acetyl]piperazine-1-carbonyl]phenyl]-1-[2-(difluoromethyl)-3-fluoro-4-(fluoromethoxy)phenyl]imidazole-4-carboxamide; formic acid

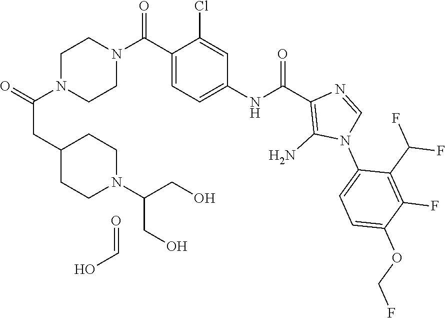

### Step 1: 2-[1-[2-[tert-butyl(dimethyl)silyl]oxy-1-[[tert-butyl(dimethyl)silyl]oxymethyl]ethyl]-4-piperidyl]acetic acid

To a solution of 4-piperidinylacetic acid hydrochloride (1 g, 5.57 mmol, 1.0 eq) and [2-[tert-butyl(dimethyl)silyl]oxy-1-[[tert-butyl(dimethyl)silyl]oxymethyl]ethyl] 4-methylbenzenesulfonate (2643 mg, 5.57 mmol, 1.0 eq) in DMSO (20 mL) was added Cs₂CO₃ (5441 mg, 16.7 mmol, 3.0 eq). The mixture was stirred at 80° C. for 16 h. The mixture was filtered, and the filtrate was poured into H₂O (30 mL) and then extracted with EtOAc (3×50 mL). The combined organic layers were washed with brine (3×30 mL), dried over Na₂SO₄, filtered, and the filtrate was concentrated under reduced pressure to afford the title compound (3.0 g, 6.73 mmol, 36% yield) as light-yellow oil.

### Step 2: 5-amino-N-[4-[4-[2-[1-[2-[tert-butyl(dimethyl)silyl]oxy-1-[[tert-butyl(dimethyl)silyl]oxymethyl]ethyl]-4-piperidyl]acetyl]piperazine-1-carbonyl]-3-chlorophenyl]-1-[2-(difluoromethyl)-3-fluoro-4-(fluoromethoxy)phenyl]imidazole-4-carboxamide

To a light-yellow stirred solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide;hydrochloride (50 mg, 0.09 mmol, 1.0 eq), 2-[1-[2-[tert-butyl(dimethyl)silyl]oxy-1-[[tert-butyl(dimethyl)silyl]oxymethyl]ethyl]-4-piperidyl]acetic acid (38 mg, 0.09 mmol, 1.0 eq) and N,N-diisopropylethylamine (0.05 mL, 0.26 mmol, 3.0 eq) in DMF (1 mL) was added HATU (49 mg, 0.13 mmol, 1.5 eq) in portions at 0° C. The resulting solution was then stirred at 20° C. for 1 h. The mixture was purified by reverse phase HPLC (0.1% FA in H₂O-MeCN, 60-70% B) to afford the title compound (20.0 mg, 0.02 mmol, 24% yield). MS: m/z=968.5 [M+H]+, ESI pos.

### Step 3: 5-amino-N-[3-chloro-4-[4-[2-[1-[2-hydroxy-1-(hydroxymethyl)ethyl]-4-piperidyl]acetyl]piperazine-1-carbonyl]phenyl]-1-[2-(difluoromethyl)-3-fluoro-4-(fluoromethoxy)phenyl]imidazole-4-carboxamide; formic acid

A solution of 5-amino-N-[4-[4-[2-[1-[2-[tert-butyl(dimethyl)silyl]oxy-1-[[tert-butyl(dimethyl)silyl]oxymethyl]ethyl]-4-piperidyl]acetyl]piperazine-1-carbonyl]-3-chlorophenyl]-1-[2-(difluoromethyl)-3-fluoro-4-(fluoromethoxy)phenyl]imidazole-4-carboxamide (20 mg, 0.02 mmol, 1.0 eq) in HCl/dioxane (1.0 mL, 2 M) was stirred at 20° C. for 1 h. The mixture was concentrated under reduced pressure. The residue was purified by prep-HPLC (column: Phenomenex luna C18 150×25 mm×10 um; condition: H₂O (FA)-MeCN; 12-42% B) to afford the title compound (3.2 mg, 0.0 mmol, 19% yield). MS: m/z=740.2 [M+H]+, ESI pos.

### Compound 346

### 5-amino-N-[3-chloro-4-[4-(3-keto-8a-methylol-1,5,6,8-tetrahydrooxazolo[3,4-a]pyrazine-7-carbonyl)piperazine-1-carbonyl]phenyl]-1-[2,3-difluoro-4-(fluoromethoxy)phenyl]imidazole-4-carboxamide 2,2,2-trifluoroacetic acid

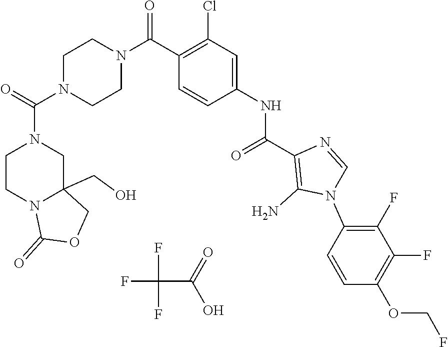 [SMILES: Nc1c(C(=O)Nc2ccc(C(=O)N3CCN(C(=O)N4CCN5C(=O)OCC5(CO)C4)CC3)c(Cl)c2)ncn1-c1ccc(OCF)c(F)c1F ; O=C(O)C(F)(F)F]

To a clear solution of N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester .1:1 2,2,2-trifluoroacetic acid (Intermediate 11, 45 mg, 61.62 μmol, 1.000 eq) in CH₂Cl₂ (2.5 mL) was added DIEA (43.8 mg, 59.2 μL, 339 μmol, 5.50 eq) followed by triphosgene (7 mg, 24.65 μmol, 0.400 eq) at 0° C. After 20 min 2-oxa-5,8-diazaspiro[3.5]nonane-5-carboxylic acid tert-butyl ester (29.61 mg, 123.23 μmol, 2.000 eq) was added and the ice-bath was removed and the reaction was stirred at RT overnight. The reaction was diluted with H₂O and extracted with CH₂Cl₂. The combined organic layer was washed with brine, dried over MgSO₄, filtered and evaporated. The residue was purified on SiO₂ using CH₂Cl₂:(CH₂Cl₂:MeOH 9:1+0.25% NH₃) as eluent to afford 47 mg intermediate.

The material was dissolved in CH₂Cl₂ (2 mL) and TFA (562 mg, 380 μL, 4.93 mmol, 80.000 eq) was added and the mixture stirred 15 h at RT. Then the mixture was concentrated under reduced pressure and the residue purified by preparative reverse phase HPLC (column: Phenomenex Gemini-NX 5u 110A, I: 100 mm, dia 30 mm) using H₂O containing 0.05% TFA/MeCN as eluent. The corresponding fractions were combined and lyophilized to afford the title compound (20 mg, 41%) MS: m/z=707.2 [M+H]⁺, ESI pos

### Compound 347

### N-(4-(4-(2-oxa-5,8-diazaspiro[3.5]nonane-5-carbonyl)piperazine-1-carbonyl)-3-chlorophenyl)-5-amino-1-(2,3-difluoro-4-(fluoromethoxy)phenyl)-1H-imidazole-4-carboxamide 2,2,2-trifluoroacetate

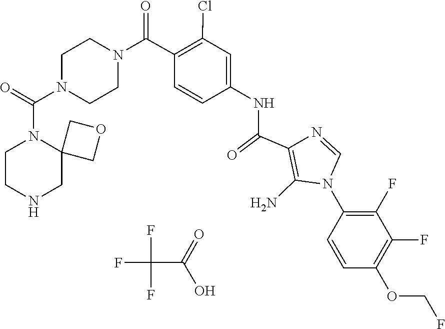 [SMILES: Nc1c(C(=O)Nc2ccc(C(=O)N3CCN(C(=O)N4CCNCC45COC5)CC3)c(Cl)c2)ncn1-c1ccc(OCF)c(F)c1F ; O=C(O)C(F)(F)F]

### Step 1: 5-amino-N-[3-chloro-4-[4-(2-oxa-5,8-diazaspiro[3.5]nonane-5-carbonyl)piperazine-1-carbonyl]phenyl]-1-[2,3-difluoro-4-(fluoromethoxy)phenyl]imidazole-4-carboxamide. 1:1.8 2,2,2-trifluoroacetic acid

To a clear solution of N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester. 1:1 2,2,2-trifluoroacetic acid (Intermediate 11, 45 mg, 61.62 μmol, 1.000 eq) in CH₂Cl₂ (2.5 mL) was added DIEA (47 mg, 64.6 μL, 369.7 μmol, 6.000 eq) followed by triphosgene (7 mg, 24.65 μmol, 0.400 eq) at 0° C. After 20 min 2-oxa-5,8-diazaspiro[3.5]nonane-8-carboxylic acid tert-butyl ester (28.13 mg, 123.23 μmol, 2.000 eq) was added. The reaction was stored overnight under refrigeration. A few drops of H₂O were added to the reaction mixture and the solvent was evaporated. The residue was dissolved in DMSO and purified by preparative reversed phase HPLC (column: Phenomenex Gemini-NX 5u 110A, I: 100 mm, dia 30 mm) using H₂O containing 0.05% TFA/MeCN as eluent. The corresponding fractions were collected and lyophilized. The residue was dissolved in CH₂Cl₂ (1 mL) and TFA (140 mg, 95 μL, 1.23 mmol, 20.000 eq) was added. The clear solution was stirred for 15 min at RT and was then stored overnight in the refrigerator. Then, the solvent was evaporated, the residue dissolved in DMSO and this solution purified by preparative reversed phase HPLC (column: Phenomenex Gemini-NX 5u 110A, I: 100 mm, dia 30 mm) using H₂O containing 0.05% TFA/MeCN as eluent. The corresponding fractions were lyophilized to afford the title compound (4 mg; 7%). MS: 707.5 [M+HCOO]⁻, ESI neg

### Compound 348

### 5-amino-N-[3-chloro-4-[4-(4-methylisonipecotoyl)piperazine-1-carbonyl]phenyl]-1-[2,3-difluoro-4-(fluoromethoxy)phenyl]imidazole-4-carboxamide. 1:1.3 2,2,2-trifluoroacetic acid

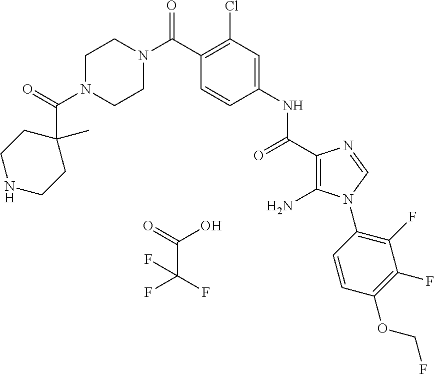 [SMILES: CC1(C(=O)N2CCN(C(=O)c3ccc(NC(=O)c4ncn(-c5ccc(OCF)c(F)c5F)c4N)cc3Cl)CC2)CCNCC1 ; O=C(O)C(F)(F)F]

Under argon atmosphere N-[5-[[3-chloro-4-(piperazine-1-carbonyl)phenyl]carbamoyl]-3-[2,3-difluoro-4-(fluoromethoxy)phenyl]imidazol-4-yl]carbamic acid tert-butyl ester;2,2,2-trifluoroacetic acid (Intermediate 11, 40 mg, 54.77 μmol, 1.000 eq) was dissolved in DMF (1.5 mL), then DIEA (28 mg, 38.26 μL, 219.08 μmol, 4.000 eq) was added, followed by 1-tert-butoxycarbonyl-4-methyl-isonipecotic acid (15 mg, 60.25 μmol, 1.100 eq) and HATU (22 mg, 60.25 μmol, 1.100 eq). The reaction was stirred at RT for 2 h. The mixture was concentrated under reduced pressure. The residue was dissolved in CH₂Cl₂ (2 mL) and TFA (499 mg, 338 μL, 4.38 mmol, 80.000 eq) was added and stirred at RT overnight. The mixture was concentrated under reduced pressure and the residue purified by preparative reversed phase HPLC (column: Phenomenex Gemini-NX 5u 110A, I: 100 mm, dia 30 mm) using H₂O containing 0.05% TFA/MeCN as eluent. The corresponding fractions combined and lyophilized to afford the title compound (27 mg, 62%) MS: m/z=678.6 [M+HCOO]⁻, ESI neg

The following compounds were prepared in analogy to the procedure above

| Compound | Reagents | MS |
|---|---|---|
| 349 | (2S,3S,4S)-1-tert-butoxycarbonyl-3- | m/z = 680.4 |
| | hydroxy-4-methyl-proline | [M + HCOO]−, |
| | | ESI neg |
| 350 | 2-tert-butoxycarbonyl-2- | m/z = 618.2 |
| | azabicyclo[2.1.1]hexane-1-carboxylic | [M + H]+, |
| | acid, deprotection using 2M HCl in | ESI pos |
| | dioxane, purification using YMC-Triart | |
| | C18 column and MeCN/H₂O + 0.1% | |
| | triethylamine as eluent | |
| 351 | Lithium 2-(2-tert-butoxycarbonyl-2,6- | m/z = 691.3 |
| | diazaspiro[3.3]heptan-6-yl)acetate | [M + HCOO]−, |
| | [2913268-38-7] | ESI neg |
| 352 | 2-tert-butoxycarbonyl-2- | M/z = 618.2 |
| | azabicyclo[2.1.1]hexane-5-carboxylic | [M + H]+ , |
| | acid, deprotection using 2M HCl in | ESI pos |
| | dioxane, purification using | |
| | MeCN/H₂O + 0.1% formic acid | |

### Compound 353

### N-[4-[4-[(2S,3aS,6aS)-1,2,3,3a,4,5,6,6a-octahydropyrrolo[2,3-c]pyrrole-2-carbonyl]piperazine-1-carbonyl]-3-chlorophenyl]-5-amino-1-[2,3-difluoro-4-(fluoromethoxy)phenyl]imidazole-4-carboxamide .1:2 2,2,2-trifluoroacetic acid

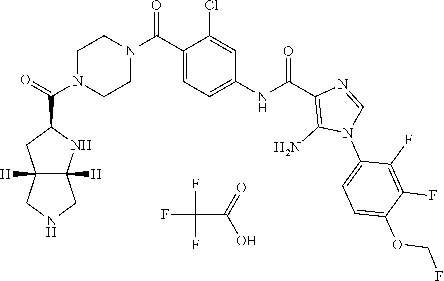 [SMILES: Nc1c(C(=O)Nc2ccc(C(=O)N3CCN(C(=O)[C@@H]4C[C@H]5CNC[C@H]5N4)CC3)c(Cl)c2)ncn1-c1ccc(OCF)c(F)c1F ; O=C(O)C(F)(F)F]

### Step 1:(2S,3aS,6aS)-2-[4-[4-[[5-amino-1-[2,3-difluoro-4-(fluoromethoxy)phenyl]imidazole-4-carbonyl]amino]-2-chloro-benzoyl]piperazine-1-carbonyl]-2,3,3a,4,6,6a-hexahydro-1H-pyrrolo[2,3-c]pyrrole-5-carboxylic acid benzyl ester .1:2 2,2,2-trifluoroacetic acid

The title compound was prepared in analogy to Compound 348 using (2S,3aS,6aS)-1-tert-butoxycarbonyl-5-carbobenzoxy-2,3,3a,4,6,6a-hexahydropyrrolo[3,4-b]pyrrole-2-carboxylic acid as reagent.

The title compound was obtained (33 mg, 59%) MS: m/z=779.4 [M−H]⁻, ESI neg

### Step 2: N-[4-[4-[(2S,3aS,6aS)-1,2,3,3a,4,5,6,6a-octahydropyrrolo[2,3-c]pyrrole-2-carbonyl]piperazine-1-carbonyl]-3-chlorophenyl]-5-amino-1-[2,3-difluoro-4-(fluoromethoxy)phenyl]imidazole-4-carboxamide .1:2 2,2,2-trifluoroacetic acid

(2S,3aS,6aS)-2-[4-[4-[[5-amino-1-[2,3-difluoro-4-(fluoromethoxy)phenyl]imidazole-4-carbonyl]amino]-2-chloro-benzoyl]piperazine-1-carbonyl]-2,3,3a,4,6,6a-hexahydro-1H-pyrrolo[2,3-c]pyrrole-5-carboxylic acid benzyl ester;2,2,2-trifluoroacetic acid (28 mg, 27 μmol, 1.0 eq) was dissolved in MeOH (1 mL) and 10% palladium on carbon (8 mg, 8.24 μmol, 0.300 eq) was added. Then a hydrogen-balloon was put on the reaction and the mixture was stirred at RT for 20 h. The reaction mixture was filtered, and the obtained solution concentrated under reduced pressure to afford the title compound (22 mg, 91%) MS: m/z=645.4 [M−H]⁻, ESI neg

### Compound 355

### 5-amino-N-[4-[4-[(1R,3s,5S,6r)-3-aminobicyclo[3.1.0]hexane-6-carbonyl]piperazine-1-carbonyl]-3-fluorophenyl]-1-[2,3-difluoro-4-(fluoromethoxy)phenyl]imidazole-4-carboxamide .1:1 2,2,2-trifluoroacetic acid

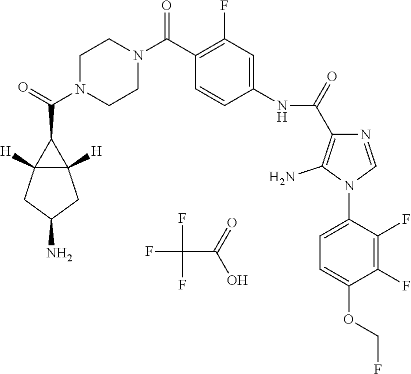 [SMILES: Nc1c(C(=O)Nc2ccc(C(=O)N3CCN(C(=O)[C@H]4[C@@H]5C[C@@H](N)C[C@@H]54)CC3)c(F)c2)ncn1-c1ccc(OCF)c(F)c1F ; O=C(O)C(F)(F)F]

### Step 1: N-[5-[[4-[4-[(1R,3s,5S,6r)-3-(benzyloxycarbonylamino)bicyclo[3.1.0]hexane-6-carbonyl]piperazine-1-carbonyl]-3-fluorophenyl]carbamoyl]-3-[2,3-difluoro-4-(fluoromethoxy)phenyl]imidazol-4-yl]carbamic acid tert-butyl ester .1:1 2,2,2-trifluoroacetic acid

Under argon atmosphere N-[3-[2,3-difluoro-4-(fluoromethoxy)phenyl]-5-[[3-fluoro-4-(piperazine-1-carbonyl)phenyl]carbamoyl]imidazol-4-yl]carbamic acid tert-butyl ester;2,2,2-trifluoroacetic acid (Intermediate 12, 54 mg, 76.43 μmol, 1.000 eq) was dissolved in anhydrous DMF (1 mL), then DIEA (39 mg, 53.4 μL, 305.7 μmol, 4.000 eq) was added, followed by (1R,3s,5S,6r)-3-(benzyloxycarbonylamino)bicyclo[3.1.0]hexane-6-carboxylic acid (Intermediate 30, 23 mg, 84.07 μmol, 1.100 eq) and HATU (31 mg, 84.07 μmol, 1.100 eq). The reaction was stirred at RT for 20 h. The mixture was directly purified by prep HPLC (column: Phenomenex Gemini-NX 5u 110A, I: 100 mm, dia: 30 mm) using MeCN/H₂O (+0.05% TFA) as eluent. The corresponding fractions were combined and lyophilized to afford the title compound (70 mg, 95%). MS: m/z=850.5 [M+H]+, ESI pos

### Step 2: N-[5-[[4-[4-[(1R,3s,5S,6r)-3-aminobicyclo[3.1.0]hexane-6-carbonyl]piperazine-1-carbonyl]-3-fluorophenyl]carbamoyl]-3-[2,3-difluoro-4-(fluoromethoxy)phenyl]imidazol-4-yl]carbamic acid tert-butyl ester .1:1 2,2,2-trifluoroacetic acid

N-[3-[2,3-difluoro-4-(fluoromethoxy)phenyl]-5-[[3-fluoro-4-[4-[(1R,3s, 5S,6r)-3-(benzyloxycarbonylamino)bicyclo[3.1.0]hexane-6-carbonyl]piperazine-1-carbonyl]phenyl]carbamoyl]imidazol-4-yl]carbamic acid tert-butyl ester;2,2,2-trifluoroacetic acid (70 mg, 72.63 μmol, 1.000 eq) was dissolved in MeOH (2 mL) and 10% palladium on carbon (23 mg, 21.79 μmol, 0.300 eq) was added. Then a balloon filled with hydrogen gas was put on the reaction and the mixture was stirred at RT for 20 h. The reaction mixture was filtered, and the obtained solution concentrated under reduced pressure to afford the title compound (54 mg, 88%). MS: m/z=716.6 [M+H]+, ESI pos

### Step 3: 5-amino-N-[4-[4-[(1R,3s,5S,6r)-3-aminobicyclo[3.1.0]hexane-6-carbonyl]piperazine-1-carbonyl]-3-fluorophenyl]-1-[2,3-difluoro-4-(fluoromethoxy)phenyl]imidazole-4-carboxamide .1:1 2,2,2-trifluoroacetic acid

N-[3-[2,3-difluoro-4-(fluoromethoxy)phenyl]-5-[[3-fluoro-4-[4-[(1S,3s,5R,6r)-3-aminobicyclo[3.1.0]hexane-6-carbonyl]piperazine-1-carbonyl]phenyl]carbamoyl]imidazol-4-yl]carbamic acid tert-butyl ester; 2,2,2-trifluoroacetic acid (10 mg, 11.81 μmol, 1.000 eq) was dissolved in CH₂Cl₂ (1.06 mL) and then TFA (67 mg, 45.5 μL, 590.6 μmol, 50.00 eq) was added and the reaction stirred at RT over-night. The mixture was purified by prep HPLC (column: Phenomenex Gemini-NX 5u 110A, I: 100 mm, dia: 30 mm) using MeCN/H₂O (+0.05% TFA) as eluent. The corresponding fractions were combined and lyophilized to afford the title compound (4.6 mg, 51%). MS: m/z=660.3 [M+HCOO]−, ESI neg

### Compound 356

### 5-amino-N-[4-[4-[(1R,5S,6r)-3-azabicyclo[3.1.0]hexane-6-carbonyl]piperazine-1-carbonyl]-3-chlorophenyl]-1-[3-fluoro-4-(fluoromethoxy)-2-(trifluoromethyl)phenyl]imidazole-4-carboxamide. 1:1.5 2,2,2-trifluoroacetic acid

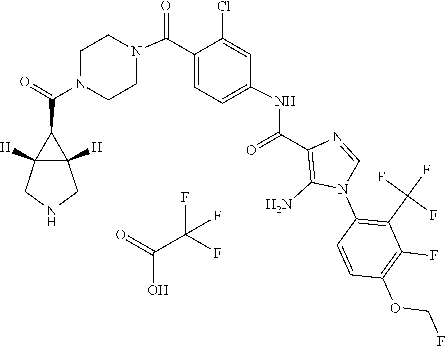 [SMILES: Nc1c(C(=O)Nc2ccc(C(=O)N3CCN(C(=O)[C@H]4[C@@H]5CNC[C@@H]54)CC3)c(Cl)c2)ncn1-c1ccc(OCF)c(F)c1C(F)(F)F ; O=C(O)C(F)(F)F]

To a solution of 5-(tert-butoxycarbonylamino)-1-[3-fluoro-4-(fluoromethoxy)-2-(trifluoromethyl)phenyl]imidazole-4-carboxylic acid (Intermediate 132, 30 mg, 66.54 μmol, 1.000 eq) and (1R,5S,6r)-6-[4-(4-amino-2-chloro-benzoyl)piperazine-1-carbonyl]-3-azabicyclo[3.1.0]hexane-3-carboxylic acid tert-butyl ester (Intermediate 22, 34 mg, 73.2 μmol, 1.100 eq) in CH₂Cl₂ (1 mL) was added 1-methylimidazole (16 mg, 15.9 μL, 199.62 μmol, 3.000 eq). The reaction mixture was cooled to 0° C. and 1-propanephosphonic acid cyclic anhydride (84 mg, 78.4 μL, 133.08 μmol, 2.000 eq) was added. After 30 min at 0° C. the reaction was heated to 40° C. for 1 h. Then, again 1-methylimidazole (16 mg, 15.9 μL, 199.62 μmol, 3.000 eq) and 1-propanephosphonic acid cyclic anhydride (84 mg, 78.4 μL, 133.1 μmol, 2.000 eq) were added and the reaction was stirred at 40° C. over-night. The solvent was evaporated and the residue directly purified by preparative reversed phase HPLC (column: Phenomenex Gemini-NX 5u 110A, I: 100 mm, dia 30 mm) using H₂O containing 0.05% TFA/MeCN as eluent. The corresponding fractions were collected, and the MeCN was partly evaporated, after which the residue was lyophilized to afford 26 mg intermediate (MS: 868.4 [M+H]+, ESI pos). The obtained material was dissolved in CH₂Cl₂ (2 mL) and TFA (778 mg, 526.3 μL, 6.83 mmol, 80.000 eq) was added and the mixture stirred 2 h at RT. The solvent was evaporated and the residue purified by preparative reversed phase HPLC (column: Phenomenex Gemini-NX 5u 110A, I: 100 mm, dia 30 mm) using H₂O containing 0.05% TFA/MeCN as eluent. The corresponding fractions were combined lyophilized to afford the title compound (16.5 mg, 30%) as lyophilized solid. MS: 712.5 [M+HCOO]−, ESI neg.

The following compounds were prepared in analogy to the procedure above

| Compound | Reagents | MS |
|---|---|---|
| 357 | 5-(tert-butoxycarbonylamino)-1-[ | m/z = 684.5 |
| | 2-cyclopropyl-3-fluoro-4-(fluoromethoxy) | [M + HCOO]−, |
| | phenyl]imidazole-4-carboxylic acid | ESI neg |
| | (Intermediate 129) | |

### Compound 358

### 5-amino-N-[4-[4-[(1R,5S,6r)-3-azabicyclo[3.1.0]hexane-6-carbonyl]piperazine-1-carbonyl]-3-chlorophenyl]-1-[6-(fluoromethoxy)-2-(trifluoromethyl)-3-pyridyl]imidazole-4-carboxamide .1:2 hydrogen chloride

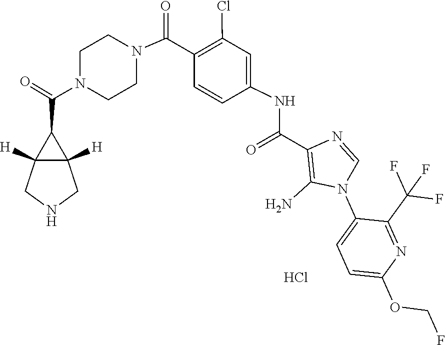 [SMILES: Nc1c(C(=O)Nc2ccc(C(=O)N3CCN(C(=O)[C@H]4[C@@H]5CNC[C@@H]54)CC3)c(Cl)c2)ncn1-c1ccc(OCF)nc1C(F)(F)F ; Cl]

### Step 1:(1R,5S,6r)-6-[4-[4-[[5-(tert-butoxycarbonylamino)-1-[6-(fluoromethoxy)-2-(trifluoromethyl)-3-pyridyl]imidazole-4-carbonyl]amino]-2-chloro-benzoyl]piperazine-1-carbonyl]-3-azabicyclo[3.1.0]hexane-3-carboxylic acid tert-butyl ester .1:1 2,2,2-trifluoroacetic acid

To a solution of 5-(tert-butoxycarbonylamino)-1-[6-(fluoromethoxy)-2-(trifluoromethyl)-3-pyridyl]imidazole-4-carboxylic acid (Intermediate 130, 55 mg, 124.31 μmol, 1.000 eq) in MeCN (2 mL) was added 1-methylimidazole (40 mg, 39.6 μL, 497.24 μmol, 4.000 eq) and N,N,N′,N′-tetramethylchloroformamidinium hexafluorophosphate (TCFH)(52 mg, 186.47 μmol, 1.500 eq) and the mixture stirred 30 min. Then, (1R,5S,6r)-6-[4-(4-amino-2-chloro-benzoyl)piperazine-1-carbonyl]-3-azabicyclo[3.1.0]hexane-3-carboxylic acid tert-butyl ester (Intermediate 22, 70 mg, 149.17 μmol, 1.200 eq) in MeCN (1 mL) was added and stirring continued for 15 h. Again TCFH (34.88 mg, 124.31 μmol, 1.000 eq) and 1-methylimidazole (30 mg, 29.7 μL, 372.93 μmol, 3.000 eq) were added and stirring continued for 5 h, after which the reaction was stored in the refrigerator for 3 days. Again TCFH (34 mg, 124.31 μmol, 1.000 eq) and 1-methylimidazole (30 mg, 29.7 μL, 372.93 μmol, 3.000 eq) were added and stirring continued for 35 h. The reaction was concentrated under reduced pressure. The residue was dissolved in EtOAc and washed with H₂O and brine. The aqueous layers were extracted with EtOAc 2 times. The combined organic layers were dried over MgSO₄, filtered and concentrated under reduced pressure. The residue was dissolved in DMSO and purified by preparative reversed phase HPLC (column: Phenomenex Gemini-NX 5u 110A, I: 100 mm, dia 30 mm) using H₂O containing 0.05% TFA/MeCN as eluent. The corresponding fractions were collected and lyophilized to afford the title compound (40 mg; 32%). MS: m/z=849.5 [M−H]−, ESI neg

### Step 2: 5-amino-N-[4-[4-[(1R,5S,6r)-3-azabicyclo[3.1.0]hexane-6-carbonyl]piperazine-1-carbonyl]-3-chlorophenyl]-1-[6-(fluoromethoxy)-2-(trifluoromethyl)-3-pyridyl]imidazole-4-carboxamide .1:2 hydrogen chloride

To a solution of (1R,5S,6r)-6-[4-[4-[[5-(tert-butoxycarbonylamino)-1-[6-(fluoromethoxy)-2-(trifluoromethyl)-3-pyridyl]imidazole-4-carbonyl]amino]-2-chloro-benzoyl]piperazine-1-carbonyl]-3-azabicyclo[3.1.0]hexane-3-carboxylic acid tert-butyl ester .1:1 2,2,2-trifluoroacetic acid (40 mg, 0.039 mmol, 1.000 eq) in CH₂Cl₂ (2 mL) was added TFA (269 mg, 182 μL, 2.36 mmol, 60.000 eq) and the mixture stirred 15 h. Then, the solvent was evaporated and the residue was dissolved in DMSO. This solution was purified by preparative reversed phase HPLC (column: Phenomenex Gemini-NX 5u 110A, I: 100 mm, dia 30 mm) using H₂O containing 0.05% HCl/MeCN as eluent. The corresponding fractions were collected and lyophilized to afford the title compound (19 mg, 67%) as lyophilized solid. MS: m/z=649.3 [M−H]−, ESI neg

### Compound 359

### 5-amino-1-[2,3-difluoro-4-(fluoromethoxy)phenyl]-N-[3-fluoro-4-(4-isonipecotoylpiperazino)sulphonylphenyl]imidazole-4-carboxamide 2,2,2-trifluoroacetic acid

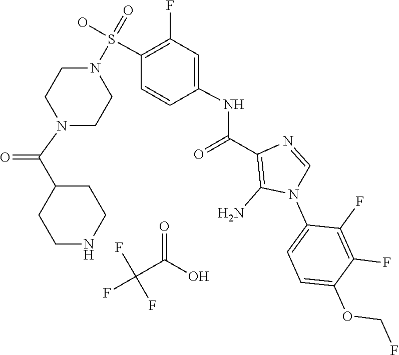

### Step 1: 4-[4-(2-fluoro-4-nitrophenyl)sulphonylpiperazine-1-carbonyl]piperidine-1-carboxylic acid tert-butyl ester

To a yellow solution of 4-(piperazine-1-carbonyl)piperidine-1-carboxylic acid tert-butyl ester (CAS [887587-18-0], 409 mg, 1.24 mmol, 1.000 eq) and DIEA (192.4 mg, 0.260 mL, 1.49 mmol, 1.203 eq) in CH₂Cl₂, extra dry (1.6 mL) was added 2-fluoro-4-nitrobenzenesulphonyl chloride (341 mg, 1.38 mmol, 1.115 eq) in portions (exothermic). The reaction was stirred at RT for 1 h. The reaction was diluted with CH₂Cl₂ and quenched with H₂O and basified using 1M aqueous sodium carbonate solution. The resulting mixture was extracted twisce with CH₂Cl₂ and the organic layers washed with H₂O/brine. The combined organic layers were dried with Na₂SO₄, filtered and concentrated under reduced pressure. The residue was purified on SiO₂ using heptane/EtOAc as eluent. The corresponding fractions were combined and concentrated under reduced pressure to afford the title compound (277 mg, 43%) as yellow foam. MS: m/z=501.1 [M+H]+, ESI pos

### Step 2: 4-[4-(4-amino-2-fluorophenyl) sulphonylpiperazine-1-carbonyl]piperidine-1-carboxylic acid tert-butyl ester

In a sealed tube a suspension of iron powder (160 mg, 2.86 mmol, 5.392 eq), NH₄Cl (149 mg, 2.79 mmol, 5.243 eq), and 4-[4-(2-fluoro-4-nitrophenyl) sulphonylpiperazine-1-carbonyl]piperidine-1-carboxylic acid tert-butyl ester (277 mg, 0.531 mmol, 1.000 eq) in EtOH (0.760 mL) and H₂O (0.120 mL) was heated to 55° C. (outer temp) and stirred 4 h, then at RT over weekend. The reaction was diluted with EtOAc and the mixture filtered through celite, washed with EtOAc and H₂O. The filtrate was treated with brine and saturated aqueous NaHCO₃. The layers were separated, the organic layer dried using Na₂SO₄, the suspension filtered and the filtrate concentrated under reduced pressure to afford the title compound (236 mg, 81%) as a brown foam. The material was used without further purificationMS: m/z=471.2 [M+H]+, ESI pos

### Step 3: 4-[4-[4-[[5-(tert-butoxycarbonylamino)-1-[2,3-difluoro-4-(fluoromethoxy)phenyl]imidazole-4-carbonyl]amino]-2-fluorophenyl]sulphonylpiperazine-1-carbonyl]piperidine-1-carboxylic acid tert-butyl ester

To a yellow suspension of 4-[4-(4-amino-2-fluorophenyl) sulphonylpiperazine-1-carbonyl]piperidine-1-carboxylic acid tert-butyl ester (51 mg, 93 μmol, 1.0 eq) and 5-(tert-butoxycarbonylamino)-1-[2,3-difluoro-4-(fluoromethoxy)phenyl]imidazole-4-carboxylic acid (Intermediate 5, 40 mg, 96 μmol, 1.0 eq) in MeCN (400 μL) was added 1-propylphosphonic acid cyclic anhydride, 50% in EtOAc (89 mg, 83 μL, 140.86 μmol, 1.511 eq) and the mixture stirred 30 min at RT. Then, N-methylimidazole (12 mg, 12 μL, 149.04 μmol, 1.599 eq) was added and stirring at RT continued 20 h. Again 1-propylphosphonic acid cyclic anhydride, 50% in EtOAc (89 mg, 83 μL, 140.86 μmol, 1.511 eq) was added and stirring at RT continued for 24 h. Then, the reaction was heated to 50° C. for 16 h, then at RT over weekend. The reaction was diluted with 400 μL MeCN, and again 1-propylphosphonic acid cyclic anhydride, 50% in EtOAc (178.2 mg, 165 μL, 280.03 μmol, 3.004 eq) and N-methylimidazole (23 mg, 23 μL, 285.67 μmol, 3.065 eq) were added and the reaction heated to 40° C. overnight. The reaction mixture was diluted with EtOAc and quenched with saturated aqueous. NaHCO₃ solution (pH ˜9) and further diluted with H₂O. The resulting mixture was extracted twice with EtOAc and the organic layers washed with brine. The combined organic layers were dried using Na₂SO₄, filtered and concentrated under reduced pressure. The residue was purified on SiO₂ using heptane/EtOAc as eluent. The corresponding fractions were combined and concentrated under reduced pressure to afford the title compound (24 mg, 26%) as colorless amorphous. The material was used without further purification. MS: m/z=840.3 [M+H]+, ESI pos

### Step 4: 5-amino-1-[2,3-difluoro-4-(fluoromethoxy)phenyl]-N-[3-fluoro-4-(4-isonipecotoylpiperazino)sulphonylphenyl]imidazole-4-carboxamide .1:1.5 2,2,2-trifluoroacetic acid

To a solution of 4-[4-[4-[[5-(tert-butoxycarbonylamino)-1-[2,3-difluoro-4-(fluoromethoxy)phenyl]imidazole-4-carbonyl]amino]-2-fluorophenyl]sulphonylpiperazine-1-carbonyl]piperidine-1-carboxylic acid tert-butyl ester (24 mg, 0.024 mmol, 1.0 eq) in 1,4-dioxane, extra dry (0.120 mL) was added 4 M HCl in dioxane (120 μL, 480 μmol, 19.8 eq) and the reaction stirred at RT for 1 h. The reaction was concentrated under reduced pressure and the residue was purified by preparative HPLC using a Gemini NX column and MeCN/H₂O+0.05% TFA as eluent. The corresponding fractions were combined and concentrated under reduced pressure. The residue was redissolved in MeCN/H₂O and lyophilized to afford the title compound (7.1 mg, 35%) as light brown powder. MS: m/z=640.2 [M+H]+, ESI pos

The following compounds were prepared in analogy to the procedure above

| Ex | Structure | Name | Reagents | MS |
|---|---|---|---|---|
| 360 | 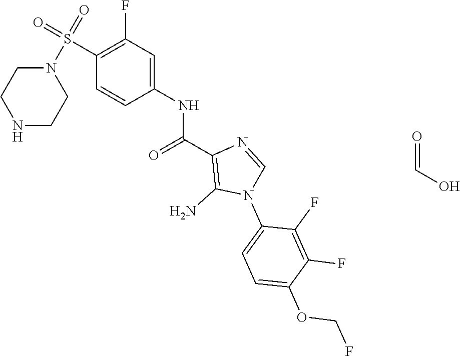 [SMILES: Nc1c(C(=O)Nc2ccc(S(=O)(=O)N3CCNCC3)c(F)c2)ncn1-c1ccc(OCF)c(F)c1F ; O=CO] | 5-amino-1-[2,3- difluoro-4- (fluoromethoxy) phenyl]- N-(3-fluoro-4- piperazinosulphonyl- phenyl)imidazole-4- carboxamide formic acid | 1-Boc-piperazine in step 1, no additional adding of NMI and T3P in step 3, purification in step 4 using YMC-Triart C18 12 nm, 5 μm, 100 × 30 mm column and MeCN/H₂O + 0.1% formic acid as eluent. | m/z = 529.1 [M + H]+, ESI pos |

### Compound 361

### 5-amino-N-[4-[4-[(1R,5S,6r)-3-azabicyclo[3.1.0]hexane-6-carbonyl]piperazine-1-carbonyl]-3-chloro-phenyl]-1-[1-(2,2-difluoroethyl)-3-(trifluoromethyl)pyrazol-4-yl]imidazole-4-carboxamide .1:1 2,2,2-trifluoroacetic acid

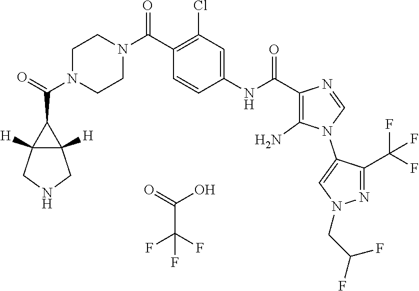 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC(F)F)nc1C(F)(F)F ; O=C(O)C(F)(F)F]

### Step 1: 4-[4-[[5-(tert-butoxycarbonylamino)-1-[1-(2,2-difluoroethyl)-3-(trifluoromethyl)pyrazol-4-yl]imidazole-4-carbonyl]amino]-2-chloro-benzoyl]piperazine-1-carboxylic acid 9H-fluoren-9-ylmethyl ester. 1:1 2,2,2-trifluoroacetic acid

To a solution of 5-(tert-butoxycarbonylamino)-1-[1-(2,2-difluoroethyl)-3-(trifluoromethyl)pyrazol-4-yl]imidazole-4-carboxylic acid (Intermediate 129, 158 mg, 0.319 mmol, 1.000 eq) and DIEA (123 mg, 167.4 μL, 958.45 μmol, 3.000 eq) in DMF (2 mL) was added HATU (145 mg, 383.38 μmol, 1.200 eq). After 15 min a solution of 4-(4-amino-2-chloro-benzoyl)piperazine-1-carboxylic acid 9H-fluoren-9-ylmethyl ester (Intermediate 28, 169 mg, 351.43 μmol, 1.100 eq) in DMF (1 mL) was added and the reaction stirred 3 days. The reaction was diluted with H₂O and extracted 3 times with EtOAc. The combined organic layers were washed with brine, dried over MgSO₄, filtered and evaporated. The residue was dissolved in DMSO. This solution was directly purified by preparative reversed phase HPLC (column: Phenomenex Gemini-NX 5u 110A, I: 100 mm, dia 30 mm) using H₂O containing 0.05% as eluent. The corresponding fractions were collected and lyophilized to afford the title compound (114 mg; 34%). MS: 869.5 [M+H]+, ESI pos

### Step 2: N-[5-[[3-chloro-4-(piperazine-1-carbonyl)phenyl]carbamoyl]-3-[1-(2,2-difluoroethyl)-3-(trifluoromethyl)pyrazol-4-yl]imidazol-4-yl]carbamic acid tert-butyl ester

To a solution of 4-[4-[[5-(tert-butoxycarbonylamino)-1-[1-(2,2-difluoroethyl)-3-(trifluoromethyl)pyrazol-4-yl]imidazole-4-carbonyl]amino]-2-chloro-benzoyl]piperazine-1-carboxylic acid 9H-fluoren-9-ylmethyl ester. 1:1 2,2,2-trifluoroacetic acid (114 mg, 110.14 μmol, 1.000 eq) in THF (1 mL) was added 2 M dimethylamine in THF (196 mg, 220 μL, 440.58 μmol, 4.000 eq) and the mixture stirred 16 h. The solvent was evaporated and the residue purified on SiO₂ using CH₂Cl₂/(CH₂Cl₂/MeOH+0.25% NH₄OH) as eluent. The corresponding fractions were concentrated under reduced pressure to afford the title compound (66 mg, 90%) as light brown foam. MS: 647.3 [M+H]+, ESI pos

### Step 3: 5-amino-N-[4-[4-[(1R,5S,6r)-3-azabicyclo[3.1.0]hexane-6-carbonyl]piperazine-1-carbonyl]-3-chloro-phenyl]-1-[1-(2,2-difluoroethyl)-3-(trifluoromethyl)pyrazol-4-yl]imidazole-4-carboxamide .1:1 2,2,2-trifluoroacetic acid

To a solution of (1R,5S,6s)-3-tert-butoxycarbonyl-3-azabicyclo[3.1.0]hexane-6-carboxylic acid (13 mg, 59.37 μmol, 1.200 eq) and DIEA (25 mg, 34.6 μL, 197.9 μmol, 4.000 eq) in DMF (500 μL) were added HATU (22 mg, 59.37 μmol, 1.200 eq) after 15 min a solution of N-[5-[[3-chloro-4-(piperazine-1-carbonyl)phenyl]carbamoyl]-3-[1-(2,2-difluoroethyl)-3-(trifluoromethyl)pyrazol-4-yl]imidazol-4-yl]carbamic acid tert-butyl ester (33 mg, 49.47 μmol, 1.000 eq) in DMF (1 mL) was added and the mixture stirred 1 h. The solvent was evaporated. The residue was dissolved in CH₂Cl₂ (1 mL) and TFA (564 mg, 381.15 μL, 4.95 mmol, 100.000 eq) was added, the mixture stirred 1 h at RT and then left standing in the refrigerator for 3 days. The solvent was evaporated. The residue was dissolved in DMSO and purified twice by preparative reversed phase HPLC (column: Phenomenex Gemini-NX 5u 110A, I: 100 mm, dia 30 mm) using H₂O containing 0.05% as eluent. The corresponding fractions were collected and lyophilized to afford the title compound (11 mg, 29%) as lyophilized solid. MS: m/z=700.3 [M+HCOO]−, ESI neg.

### Compound 362

### 5-amino-N-[3-chloro-4-[4-[(2S,4S)-4-methylpyrrolidine-2-carbonyl]piperazine-1-carbonyl]phenyl]-1-[2-(difluoromethyl)-3-fluoro-4-(fluoromethoxy)phenyl]imidazole-4-carboxamide;formic acid

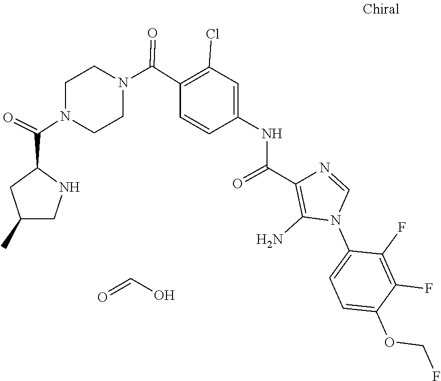 [SMILES: C[C@@H]1CN[C@H](C(=O)N2CCN(C(=O)c3ccc(NC(=O)c4ncn(-c5ccc(OCF)c(F)c5F)c4N)cc3Cl)CC2)C1 ; O=CO]

### Step 1: tert-butyl(2S,4S)-2-[4-[4-[[5-amino-1-[2-(difluoromethyl)-3-fluoro-4-(fluoromethoxy)phenyl]imidazole-4-carbonyl]amino]-2-chloro-benzoyl]piperazine-1-carbonyl]-4-methyl-pyrrolidine-1-carboxylate

To a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide (Intermediate 133, 50 mg, 0.09 mmol, 1.0 eq) in CH₂Cl₂ (3 mL) were added (2S,4S)-1-tert-butoxycarbonyl-4-methyl-pyrrolidine-2-carboxylic acid (42 mg, 0.18 mmol, 2.0 eq), 1,3,5,2,4,6-trioxatriphosphorinane, 2,4,6-tributyl-, 2,4,6-trioxide (70.3 mg, 0.18 mmol, 2.0 eq) and N,N-diisopropylethylamine (35 mg, 0.28 mmol, 3.0 eq), the resultant mixture was stirred at 25° C. for 1 h. The solvent was removed under reduced pressure, the residue was diluted with EtOAc (5 mL) and washed with H₂O (4×5 mL), dried over Na₂SO₄ and concentrated under reduced pressure to give the title compound (60 mg, 0.08 mmol, 86% yield). MS: m/z=652.0 [(M−Boc+H)+])

### Step 2: 5-amino-N-[3-chloro-4-[4-[(2S,4S)-4-methylpyrrolidine-2-carbonyl]piperazine-1-carbonyl]phenyl]-1-[2-(difluoromethyl)-3-fluoro-4-(fluoromethoxy)phenyl]imidazole-4-carboxamide; formic acid

A solution of tert-butyl(2S,4S)-2-[4-[4-[[5-amino-1-[2-(difluoromethyl)-3-fluoro-4-(fluoromethoxy)phenyl]imidazole-4-carbonyl]amino]-2-chloro-benzoyl]piperazine-1-carbonyl]-4-methyl-pyrrolidine-1-carboxylate (60 mg, 0.08 mmol, 1.0 eq) and trifluoroacetic acid (1.0 mL, 12.98 mmol, 162.72 eq) in CH₂Cl₂ (4 mL) was stirred at 25° C. for 1 h. The resulting solution was concentrated under reduced pressure. The crude product was purified by Prep-HPLC (Gemini-C18 150×21.2 mm, 5 um) using CAN/H₂O+ as eluent to afford the title compound (22.8 mg, 0.03 mmol, 41% yield). MS: m/z=652.1 [M+H]+, ESI pos

The following compounds were prepared in analogy to the procedure above

| Compound | Reagents | MS |
|---|---|---|
| 363 | (2S,4R)-1-tert-butoxycarbonyl-4-methyl- | m/z = 652.1 |
| | pyrrolidine-2-carboxylic acid, purification | [M + H] |
| | using MeCN/H₂O + 0.1% TFA as eluent | |

### Compound 364

### 5-Amino-N-[4-[4-[(1S,5R,6r)-3-azabicyclo[3.1.0]hexane-6-carbonyl]piperazine-1-carbonyl]-3-methylphenyl]-1-[2,3-difluoro-4-(fluoromethoxy)phenyl]imidazole-4-carboxamide;2,2,2-trifluoroacetic acid

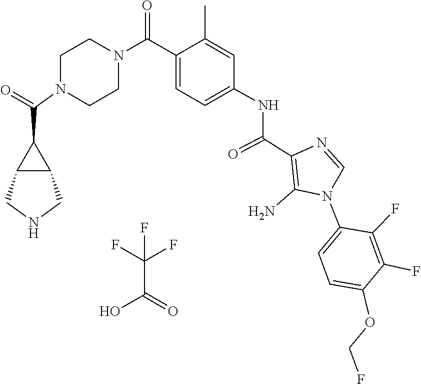 [SMILES: Cc1cc(NC(=O)c2ncn(-c3ccc(OCF)c(F)c3F)c2N)ccc1C(=O)N1CCN(C(=O)[C@H]2[C@@H]3CNC[C@@H]32)CC1 ; O=C(O)C(F)(F)F]

### Step 1: tert-Butyl (1R,5S,6r)-6-[4-(2-methyl-4-nitrobenzoyl)piperazine-1-carbonyl]-3-azabicyclo[3.1.0]hexane-3-carboxylate

A solution of (2-methyl-4-nitrophenyl)-piperazin-1-yl-methanone ([1305571-38-3], 1.8 g, 7.2 mmol, 1.0 eq), (1R,5S,6r)-3-tert-butoxycarbonyl-3-azabicyclo[3.1.0]hexane-6-carboxylic acid ([927679-54-7], 1.64 g, 7.22 mmol, 1.0 eq), propylphosphonic anhydride (11.49 g, 36.11 mmol, 5.0 eq) and diisopropylethylamine (4.67 g, 36.1 mmol, 5.0 eq) was stirred at 25° C. for 3 h. The solvent was removed under reduced pressure. The residue was diluted with CH₂Cl₂ (30 mL), washed with H₂O (4×20 mL), dried over Na₂SO₄ and concentrated under reduced pressure to yield the title compound (2.6 g, 76% yield). MS: 403.0 [M−tBu+H]+, ESI pos

### Step 2: tert-Butyl (1R,5S,6r)-6-[4-(4-amino-2-methyl-benzoyl)piperazine-1-carbonyl]-3-azabicyclo[3.1.0]hexane-3-carboxylate

A mixture of tert-butyl(1R,5S,6r)-6-[4-(2-methyl-4-nitrobenzoyl)piperazine-1-carbonyl]-3-azabicyclo[3.1.0]hexane-3-carboxylate (2.6 g, 5.7 mmol, 1.0 eq), NH₄Cl (3.03 g, 56.7 mmol, 10.0 eq), and Fe dust (3.18 g, 56.7 mmol, 10.0 eq) in EtOH (15 mL) and H₂O (5 mL) was heated to 50° C. for 2 h. The solid was filtered out. The filtrate was concentrated under reduced pressure. The residue was diluted with H₂O and then extracted with 2×50 mL of EtOAc. The organic layers were combined, washed with brine, dried and concentrated under reduced pressure to afford the title compound (2100 mg, 4.9 mmol, 86% yield). MS (ESI): m/z=373 [M−t−Bu+H]+

### Step 3: tert-Butyl (1S,5R,6r)-6-[4-[4-[[5-amino-1-[2,3-difluoro-4-(fluoromethoxy)phenyl]imidazole-4-carbonyl]amino]-2-methyl-benzoyl]piperazine-1-carbonyl]-3-azabicyclo[3.1.0]hexane-3-carboxylate

To a solution of tert-butyl(1S,5R,6r)-6-[4-(4-amino-2-methyl-benzoyl)piperazine-1-carbonyl]-3-azabicyclo[3.1.0]hexane-3-carboxylate (56 mg, 0.13 mmol, 0.8 eq) in CH₂Cl₂ (2 mL) were added pyridine (0.26 mL, 3.3 mmol, 20 eq) and 5-amino-1-[2,3-difluoro-4-(fluoromethoxy)phenyl]imidazole-4-carbonyl chloride (50 mg, 0.16 mmol, 1.0 eq) under N₂ atmosphere. The resultant mixture was stirred at 20° C. for 1 h and then diluted with EtOAc (30 mL) washed with H₂O (3×15 mL), dried over Na₂SO₄, and concentrated under reduced pressure. The residue was purified by flash column chromatography (CH₂Cl₂:MeOH=95:5) to afford the title compound (80 mg, 0.11 mmol, 49% yield). MS: 698.2 [M+H]+ ESI pos

### Step 4: 5-Amino-N-[4-[4-[(1S,5R,6r)-3-azabicyclo[3.1.0]hexane-6-carbonyl]piperazine-1-carbonyl]-3-methylphenyl]-1-[2,3-difluoro-4-(fluoromethoxy)phenyl]imidazole-4-carboxamide;2,2,2-trifluoroacetic acid

A solution of tert-butyl(1S,5R,6r)-6-[4-[4-[[5-amino-1-[2,3-difluoro-4-(fluoromethoxy)phenyl]imidazole-4-carbonyl]amino]-2-methyl-benzoyl]piperazine-1-carbonyl]-3-azabicyclo[3.1.0]hexane-3-carboxylate (49 mg, 0.07 mmol, 1.0 eq) and trifluoroacetic acid (0.25 mL) in CH₂Cl₂ (1 mL) was stirred at 25° C. for 0.5 h. The resulting solution was concentrated under reduced pressure. The residue was purified by Prep-HPLC (Gemini-C18 150×21.2 mm, 5 um, MeCN— H₂O (0.1% TFA), 35-40% B) to afford the title compound (17.5 mg, 0.02 mmol, 34% yield). MS (ESI): m/z 598.1 [M+H]+

### Compound 365

### 5-Amino-N-[3-chloro-4-[4-(piperidine-4-carbonyl)piperazine-1-carbonyl]phenyl]-1-[2,3-difluoro-4-[1-(2-methoxyethyl)-3-methyl-pyrazol-4-yl]phenyl]imidazole-4-carboxamide;2,2,2-trifluoroacetic acid

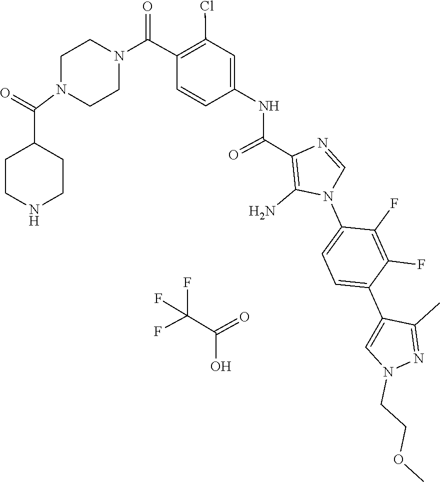 [SMILES: COCCn1cc(-c2ccc(-n3cnc(C(=O)Nc4ccc(C(=O)N5CCN(C(=O)C6CCNCC6)CC5)c(Cl)c4)c3N)c(F)c2F)c(C)n1 ; O=C(O)C(F)(F)F]

### Step 1: 5-Amino-1-[2,3-difluoro-4-[1-(2-methoxyethyl)-3-methyl-pyrazol-4-yl]phenyl]imidazole-4-carbonyl chloride

A solution of 5-amino-1-[2,3-difluoro-4-[1-(2-methoxyethyl)-3-methyl-pyrazol-4-yl]phenyl]imidazole-4-carboxylic acid (Intermediate 48, 50 mg, 0.13 mmol, 1.0 eq) in thionyl chloride (1.43 mL, 19.7 mmol, 148 eq) was stirred at 25° C. for 1 h. The resulting solution was concentrated under reduced pressure to give the title compound (50 mg, 0.13 mmol, 48% yield) as a yellow oil. MS: m/z=392.1 [(M+H)+] ESI pos

### Step 2: tert-Butyl 4-[4-[4-[[5-amino-1-[2,3-difluoro-4-[1-(2-methoxyethyl)-3-methyl-pyrazol-4-yl]phenyl]imidazole-4-carbonyl]amino]-2-chlorobenzoyl]piperazine-1-carbonyl]piperidine-1-carboxylate

To a solution of tert-butyl 4-[4-(4-amino-2-chlorobenzoyl)piperazine-1-carbonyl]piperidine-1-carboxylate (50 mg, 0.11 mmol, 0.7 eq) in CH₂Cl₂ (2 mL) were added pyridine (0.26 mL, 3.18 mmol, 20.0 eq) and 5-amino-1-[2,3-difluoro-4-[1-(2-methoxyethyl)-3-methyl-pyrazol-4-yl]phenyl]imidazole-4-carbonyl chloride (63 mg, 0.16 mmol, 1.0 eq) under N₂ atmosphere. The resultant mixture was stirred at 25° C. for 1 h. The mixture was diluted with EtOAc (20 mL) and washed with H₂O (3×15 mL), dried over Na₂SO₄ and concentrated under reduced pressure and the residue was purified by prep-TLC (CH₂Cl₂:MeOH 93:7) to afford the title compound (30 mg, 0.04 mmol, 23% yield). MS: 810.4 [M+H]+ ESI pos

### Step 3: 5-Amino-N-[3-chloro-4-[4-(piperidine-4-carbonyl)piperazine-1-carbonyl]phenyl]-1-[2,3-difluoro-4-[1-(2-methoxyethyl)-3-methyl-pyrazol-4-yl]phenyl]imidazole-4-carboxamide;2,2,2-trifluoroacetic acid

To a solution of tert-butyl 4-[4-[4-[[5-amino-1-[2,3-difluoro-4-[1-(2-methoxyethyl)-3-methyl-pyrazol-4-yl]phenyl]imidazole-4-carbonyl]amino]-2-chlorobenzoyl]piperazine-1-carbonyl]piperidine-1-carboxylate (30 mg, 0.04 mmol, 1.0 eq) in CH₂Cl₂ (1 mL) was added trifluoroacetic acid (1.0 mL, 13 mmol, 350 eq) under N₂ atmosphere. The resultant mixture was stirred at 25° C. for 0.5 h. The solvent was removed under reduced pressure, the residue was purified by prep-HPLC [Gemini-C18, 150×21.2 mm, 5 μm, MeCN-H₂O (0.1% TFA), 25-50% B] to afford the title compound (12.2 mg, 0.01 mmol, 48% yield). MS: 710.3 [M+H]+ ESI pos

### Compound 366

### N-[4-[4-[(3aS,6aR)-2-(2-hydroxyethyl)-1,3,3a,4,6,6a-hexahydropyrrolo[3,4-c]pyrrole-5-carbonyl]piperidine-1-carbonyl]-3-chloro-phenyl]-5-amino-1-[2,3-difluoro-4-(fluoromethoxy)phenyl]imidazole-4-carboxamide;2,2,2-trifluoroacetic acid

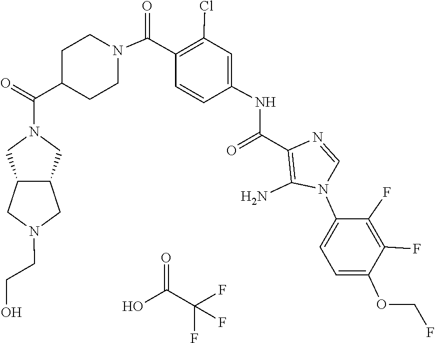 [SMILES: Nc1c(C(=O)Nc2ccc(C(=O)N3CCC(C(=O)N4C[C@H]5CN(CCO)C[C@H]5C4)CC3)c(Cl)c2)ncn1-c1ccc(OCF)c(F)c1F ; O=C(O)C(F)(F)F]

### Step 1: tert-butyl(3aR,6aS)-2-[1-[4-[[5-amino-1-[2,3-difluoro-4-(fluoromethoxy)phenyl]imidazole-4-carbonyl]amino]-2-chloro-benzoyl]piperidine-4-carbonyl]-1,3,3a,4,6,6a-hexahydropyrrolo[3,4-c]pyrrole-5-carboxylate

A solution of tert-butyl(3aR,6aS)-2-[1-(4-amino-2-chloro-benzoyl)piperidine-4-carbonyl]-1,3,3a,4,6,6a-hexahydropyrrolo[3,4-c]pyrrole-5-carboxylate (Intermediate 59, 100 mg, 0.21 mmol, 1.0 eq), 5-amino-1-[2,3-difluoro-4-(fluoromethoxy)phenyl]imidazole-4-carbonyl chloride (64 mg, 0.21 mmol, 1.0 eq) and pyridine (0.17 mL, 2.1 mmol, 10.0 eq) in DCM (4 mL) was stirred at 20° C. for 3 h. The resulting solution was concentrated under vacuum. The residue was applied on a silica gel column and eluted with MeOH/DCM (1/20) to afford the title compound (150 mg, 0.2 mmol, 95.89% yield). MS: 746.2 [M+H]+

### Step 2: N-[4-[4-[(3aS,6aR)-2,3,3a,4,6,6a-hexahydro-1H-pyrrolo[3,4-c]pyrrole-5-carbonyl]piperidine-1-carbonyl]-3-chloro-phenyl]-5-amino-1-[2,3-difluoro-4-(fluoromethoxy)phenyl]imidazole-4-carboxamide

A solution of tert-butyl(3aR,6aS)-2-[1-[4-[[5-amino-1-[2,3-difluoro-4-(fluoromethoxy)phenyl]imidazole-4-carbonyl]amino]-2-chloro-benzoyl]piperidine-4-carbonyl]-1,3,3a,4,6,6a-hexahydropyrrolo[3,4-c]pyrrole-5-carboxylate (100 mg, 0.13 mmol, 1.0 eq) and trifluoroacetic acid (1.0 mL, 12.98 mmol, 96.85 eq) in DCM (3 mL) was stirred at 20° C. for 1 h.

The mixture was concentrated in vacuo to afford the title compound (80 mg, 92% yield) as brown oil, which was used without further purification. MS: 646 [M+H]+

### Step 3: N-[4-[4-[(3aS,6aR)-2-(2-hydroxyethyl)-1,3,3a,4,6,6a-hexahydropyrrolo[3,4-c]pyrrole-5-carbonyl]piperidine-1-carbonyl]-3-chloro-phenyl]-5-amino-1-[2,3-difluoro-4-(fluoromethoxy)phenyl]imidazole-4-carboxamide;2,2,2-trifluoroacetic acid

A solution of N-[4-[4-[(3aS,6aR)-2,3,3a,4,6,6a-hexahydro-1H-pyrrolo[3,4-c]pyrrole-5-carbonyl]piperidine-1-carbonyl]-3-chloro-phenyl]-5-amino-1-[2,3-difluoro-4-(fluoromethoxy)phenyl]imidazole-4-carboxamide (50 mg, 0.08 mmol, 1.0 eq), sodium acetate (0.06 mL, 0.77 mmol, 10.0 eq), 2-hydroxyacetaldehyd (23 mg, 0.39 mmol, 5.0 eq) and sodium cyanoborohydride (24 mg, 0.39 mmol, 5.0 eq) in methanol (3 mL) was stirred at 20° C. for 6 h.

The resulting solution was concentrated under vacuum. The residue was purified by Prep-HPLC (Gemini-C18 150×19 mm, 5 um, ACN— H₂O (0.1% TFA), 35-45) to afford the title compound (11.7 mg, 0.01 mmol, 22.4% yield). MS: 712.2 [M+Na]+

### Compound 367

### 5-Amino-N-[4-[4-[3-(azetidin-3-yloxy) azetidine-1-carbonyl]piperidine-1-carbonyl]-3-chlorophenyl]-1-[2,3-difluoro-4-(fluoromethoxy)phenyl]imidazole-4-carboxamide;2,2,2-trifluoroacetic acid

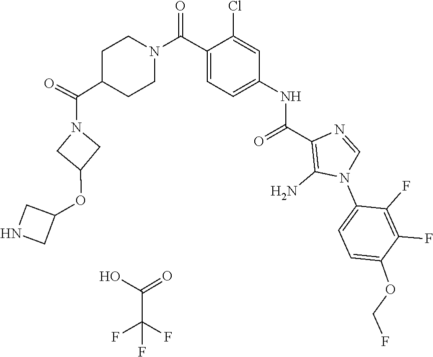 [SMILES: Nc1c(C(=O)Nc2ccc(C(=O)N3CCC(C(=O)N4CC(OC5CNC5)C4)CC3)c(Cl)c2)ncn1-c1ccc(OCF)c(F)c1F ; O=C(O)C(F)(F)F]

### Step 1: tert-Butyl 3-(Azetidin-3-yloxy) azetidine-1-carboxylate

To a solution of benzyl 3-(1-tert-butoxycarbonylazetidin-3-yl)oxyazetidine-1-carboxylate ([2920758-35-4], 1850.0 mg, 5.1 mmol, 1.0 eq) in MeOH (120 mL) was added Pd (carbon-supported, spherical particles)(430 mg, 0.16 mmol, 0.03 eq) under hydrogen atmosphere, the resultant mixture was stirred at 25° C. for 20 h. The solid was filtered off and solvent was removed under reduced pressure to give the title compound (630 mg, 2.76 mmol, 54% yield) as a light-yellow gum. MS: 229.1 [M+H]+ ESI pos

### Step 2: tert-Butyl 3-[1-[1-[4-[[5-amino-1-[2,3-difluoro-4-(fluoromethoxy)phenyl]imidazole-4-carbonyl]amino]-2-chlorobenzoyl]piperidine-4-carbonyl]azetidin-3-yl]oxyazetidine-1-carboxylate

To a solution of 1-[4-[[5-amino-1-[2,3-difluoro-4-(fluoromethoxy)phenyl]imidazole-4-carbonyl]amino]-2-chlorobenzoyl]piperidine-4-carboxylic acid (Intermediate 76, 80 mg, 0.14 mmol, 1.0 eq) and tert-butyl 3-(azetidin-3-yloxy) azetidine-1-carboxylate (33 mg, 0.14 mmol, 1.0 eq) in CH₂Cl₂ (1 mL) was added 2-(7-azabenzotriazol-1-yl)-N,N,N′,N′-tetramethyluronium hexafluorophosphate (110 mg, 0.29 mmol, 2.0 eq) and N,N-diisopropylethylamine (0.08 mL, 0.43 mmol, 3.0 eq) stirred at 25° C. for 2 h. The reaction was quenched with H₂O (25 mL). The resulting solution was extracted with EtOAc (3×20 mL). The combined organic phases were dried over Na₂SO₄ and concentrated under reduced pressure. The residue was purified by flash column to afford the title compound (90 mg, 0.12 mmol, 78% yield) as a yellow oil. MS: 761.3 [M+H]+, ESI pos

### Step 3: 5-Amino-N-[4-[4-[3-(azetidin-3-yloxy) azetidine-1-carbonyl]piperidine-1-carbonyl]-3-chlorophenyl]-1-[2,3-difluoro-4-(fluoromethoxy)phenyl]imidazole-4-carboxamide;2,2,2-trifluoroacetic acid

To a solution of tert-butyl 3-[1-[1-[4-[[5-amino-1-[2,3-difluoro-4-(fluoromethoxy)phenyl]imidazole-4-carbonyl]amino]-2-chlorobenzoyl]piperidine-4-carbonyl]azetidin-3-yl]oxyazetidine-1-carboxylate (53 mg, 0.07 mmol, 1.0 eq) and trifluoroacetic acid (1.0 mL, 13 mmol, 190 eq) in CH₂Cl₂ (3 mL) was stirred at 25° C. for 1 h. The resulting solution was concentrated under reduced pressure. The residue was purified by Prep-HPLC (Gemini-C18 150×21.2 mm, 5 μm, MeCN— H₂O (0.1% TFA), 25-40% B) to afford the title compound (28.7 mg, 0.04 mmol, 53% yield). MS: 661.9 [M+H+] ESI pos

### Compound 368

### 5-Amino-N-[3-chloro-4-[4-(piperazine-1-carbonyl)piperazine-1-carbonyl]phenyl]-1-(4-fluoro-1-methyl-indazol-5-yl)imidazole-4-carboxamide;2,2,2-trifluoroacetic acid

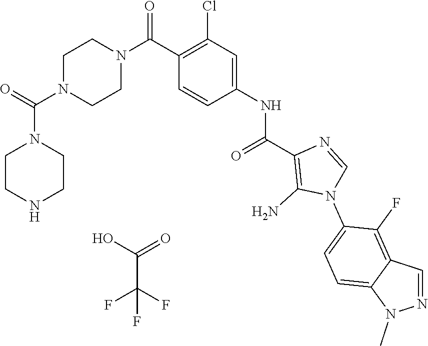 [SMILES: Cn1ncc2c(F)c(-n3cnc(C(=O)Nc4ccc(C(=O)N5CCN(C(=O)N6CCNCC6)CC5)c(Cl)c4)c3N)ccc21 ; O=C(O)C(F)(F)F]

### Step 1: tert-Butyl 4-[4-(2-chloro-4-nitrobenzoyl)piperazine-1-carbonyl]piperazine-1-carboxylate

A solution of (2-chloro-4-nitrophenyl)-piperazin-1-yl-methanone ([59939-73-0], 3 g, 11.12 mmol, 1.0 eq), triphosgene (1.65 g, 5.56 mmol, 0.5 eq), NEt₃ (15.51 mL, 111.24 mmol, 10.0 eq), and 1-BOC-piperazine (2.07 g, 11.12 mmol, 1.0 eq) in CH₂Cl₂ (20 mL) was stirred at 20° C. for 1 h. The resulting solution was concentrated under reduced pressure. The residue was applied on a silica gel column and eluted with i-PrOH/PE (99/1) to give the title compound (3 g, 6.22 mmol, 55% yield). MS (ESI): m/z=426 [M+H−tBu]+

### Step 2: tert-Butyl 4-[4-(4-amino-2-chlorobenzoyl)piperazine-1-carbonyl]piperazine-1-carboxylate

A mixture of tert-butyl 4-[4-(2-chloro-4-nitrobenzoyl)piperazine-1-carbonyl]piperazine-1-carboxylate (3 g, 6.22 mmol, 1.0 eq), NH₄Cl (3.33 g, 62.25 mmol, 10.0 eq) andiron dust (3.49 g, 62.25 mmol, 10.0 eq) in EtOH (8 mL) and H₂O (3 mL) was heated to 50° C. for 1 h. The solid was filtered out, and the filtrate was concentrated under reduced pressure. The residue was diluted with H₂O and then extracted with EtOAc (2×50 mL). The organic layers were combined, washed with brine, dried and concentrated under reduced pressure to afford the title compound (2 g, 4.43 mmol, 71% yield). MS (ESI): m/z=452 [M+H]+

### Step 3: tert-Butyl 4-[4-[2-chloro-4-[[5-[(E)-dimethylaminomethyleneamino]-1-(4-fluoro-1-methyl-indazol-5-yl)imidazole-4-carbonyl]amino]benzoyl]piperazine-1-carbonyl]piperazine-1-carboxylate

Pyridine (0.12 mL, 1.43 mmol, 5.0 eq) and tert-butyl 4-[4-(4-amino-2-chlorobenzoyl)piperazine-1-carbonyl]piperazine-1-carboxylate (129 mg, 0.29 mmol, 1.0 eq) were added to a mixture of 5-[(E)-dimethylaminomethyleneamino]-1-(4-fluoro-1-methyl-indazol-5-yl)imidazole-4-carbonyl chloride (Intermediate 49, 100 mg, 0.29 mmol, 1.0 eq) in CH₂Cl₂ (5 mL) in turn. The mixture was stirred at 25° C. for 1 h. The reaction was taken up in CH₂Cl₂ (30 ml) and the organics washed with 30 ml H₂O then 30 ml brine. The organics were then separated and dried (MgSO₄) before concentration to dryness. The crude was then purified by flash column chromatography eluting 0˜10% MeOH in CH₂Cl₂ to afford the title compound (180 mg, 0.24 mmol, 62% yield). MS: 764.2 [M+H]+

### Step 4: tert-Butyl 4-[4-[4-[[5-amino-1-(4-fluoro-1-methyl-indazol-5-yl)imidazole-4-carbonyl]amino]-2-chlorobenzoyl]piperazine-1-carbonyl]piperazine-1-carboxylate

A solution of NaOH (39 mg, 0.98 mmol, 5.0 eq) in H₂O (1 mL) was added to a solution of tert-butyl 4-[4-[2-chloro-4-[[5-[(E)-dimethylaminomethyleneamino]-1-(4-fluoro-1-methyl-indazol-5-yl)imidazole-4-carbonyl]amino]benzoyl]piperazine-1-carbonyl]piperazine-1-carboxylate (150 mg, 0.2 mmol, 1.0 eq) in MeOH (5 mL), then mixture was heated to 50° C. and stirred for 1 h. The reaction was concentrated to dryness and the residue was taken up in CH₂Cl₂ (20 ml) and the organics washed with 20 ml H₂O then 20 ml brine. The organics were then separated and dried (MgSO₄), then concentrated under reduced pressure to afford the title compound (100 mg, 0.14 mmol, 66% yield). The crude product was directly used to the next step without further purification. MS: 709.3 [M+H+]

### Step 6: 5-Amino-N-[3-chloro-4-[4-(piperazine-1-carbonyl)piperazine-1-carbonyl]phenyl]-1-(4-fluoro-1-methyl-indazol-5-yl)imidazole-4-carboxamide;2,2,2-trifluoroacetic acid

Trifluoroacetic acid (723.5 mg, 6.35 mmol, 50.0 eq) was added to a solution of tert-butyl 4-[4-[4-[[5-amino-1-(4-fluoro-1-methyl-indazol-5-yl)imidazole-4-carbonyl]amino]-2-chlorobenzoyl]piperazine-1-carbonyl]piperazine-1-carboxylate (90 mg, 0.13 mmol, 1.0 eq) in CH₂Cl₂ (5 mL), the mixture was stirred at 25° C. for 1 h. The mixture was concentrated under reduced pressure and purified by prep-HPLC: column Gemini-C18 150×21.2 mm, um MeCN— H₂O (0.1% TFA) to afford the title compound (60.1 mg, 0.08 mmol, 65% yield). MS: 609.1 [M+H]+ ESI pos

### Compound 369

### 5-amino-N-[3-chloro-4-[4-(piperidine-4-carbonyl)piperazine-1-carbonyl]phenyl]-1-[4-(cyclopropoxy)-2,3-difluoro-phenyl]imidazole-4-carboxamide;2,2,2-trifluoroacetic acid

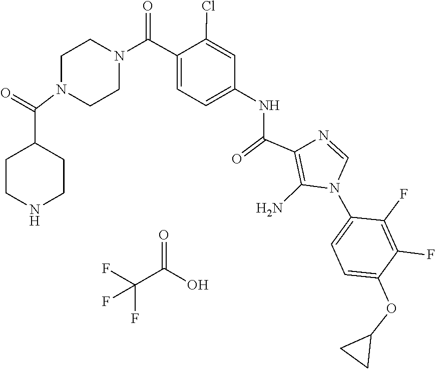 [SMILES: Nc1c(C(=O)Nc2ccc(C(=O)N3CCN(C(=O)C4CCNCC4)CC3)c(Cl)c2)ncn1-c1ccc(OC2CC2)c(F)c1F ; O=C(O)C(F)(F)F]

### Step 1: 5-amino-1-[4-(cyclopropoxy)-2,3-difluoro-phenyl]imidazole-4-carbonyl chloride

To 5-amino-1-[4-(cyclopropoxy)-2,3-difluoro-phenyl]imidazole-4-carboxylic acid (Intermediate 134, 100 mg, 0.34 mmol, 1.0 eq) under N₂ was added thionyl chloride (40 mg, 0.34 mmol, 1.0 eq) at 25° C., the solution was stirred at 25° C. for 1 h under N₂. The resultant solution was evaporated to dryness and azeotroped with DCM to give the title compound (100 mg, 0.32 mmol, 65.2% yield), which was used without further purification. MS: 310.1 [M−Cl+OCH₃+H]+ (MeOH quench)

### Step 2: tert-butyl 4-[4-[4-[[5-amino-1-[4-(cyclopropoxy)-2,3-difluoro-phenyl]imidazole-4-carbonyl]amino]-2-chloro-benzoyl]piperazine-1-carbonyl]piperidine-1-carboxylate

To a solution of tert-butyl 4-[4-(4-amino-2-chloro-benzoyl)piperazine-1-carbonyl]piperidine-1-carboxylate ([2489205-72-1], 100 mg, 0.22 mmol, 0.7 eq) and pyridine (504 mg, 6.38 mmol, 20.0 eq) in DCM (3 mL) was added a suspension of 5-amino-1-[4-(cyclopropoxy)-2,3-difluoro-phenyl]imidazole-4-carbonyl chloride (100 mg, 0.32 mmol, 1.0 eq) in DCM (3 mL) at 25° C. The mixture was stirred at 25° C. for 12 h under N₂. The resulting solution was evaporated under reduced pressure. The residue was purified by column chromatography (silica gel, 0 to 10% MeOH in DCM) to give the title compound (100 mg, 0.14 mmol, 38.26% yield). MS: 627.8 [M+H−tBu]+

### Step 3: 5-amino-N-[3-chloro-4-[4-(piperidine-4-carbonyl)piperazine-1-carbonyl]phenyl]-1-[4-(cyclopropoxy)-2,3-difluoro-phenyl]imidazole-4-carboxamide;2,2,2-trifluoroacetic acid

A solution of tert-butyl 4-[4-[4-[[5-amino-1-[4-(cyclopropoxy)-2,3-difluoro-phenyl]imidazole-4-carbonyl]amino]-2-chloro-benzoyl]piperazine-1-carbonyl]piperidine-1-carboxylate (80 mg, 0.11 mmol, 1.0 eq) in trifluoroacetic acid (1.0 mL, 0.11 mmol, 1.0 eq) the reaction was stirred at 25° C. C for 1 h The reaction was concentrated to dryness and the crude material purified by Prep-HPLC (Gemini-C18 150×21.2 mm, 5 um, ACN— H₂O (0.1% TFA), 15-40) to afford the title compound (55.5 mg, 0.07 mmol, 65.87% yield). MS [M+H]+: 627.9

### Compound 370

### 5-amino-N-[3-chloro-4-[4-(piperidine-4-carbonyl)piperazine-1-carbonyl]phenyl]-1-(4-fluoro-1-methyl-indol-5-yl)imidazole-4-carboxamide;2,2,2-trifluoroacetic acid

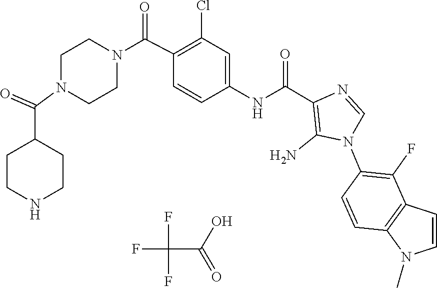 [SMILES: Cn1ccc2c(F)c(-n3cnc(C(=O)Nc4ccc(C(=O)N5CCN(C(=O)C6CCNCC6)CC5)c(Cl)c4)c3N)ccc21 ; O=C(O)C(F)(F)F]

### Step 1: 5-Amino-1-(4-fluoro-1-methyl-indol-5-yl)imidazole-4-carboxylic acid

To NaOH (363 mg, 9.1 mmol, 5.0 eq) was added to ethyl 5-amino-1-(4-fluoro-1-methyl-indol-5-yl)imidazole-4-carboxylate (Intermediate 50, 550 mg, 1.82 mmol, 1.0 eq) in i-PrOH (4 mL) and H₂O (2 mL). The resultant mixture was stirred at 50° C. for 2 h. The mixture was adjusted to pH 5-6 with 2N aqueous HCl and extracted with EtOAc. The aqueous layer was extracted again with EtOAc. The combined organic layer was dried over Na₂SO₄ and concentrated under reduced pressure the title compound (300 mg, 1.09 mmol, 60% yield). MS: 275.1 [M+H]+, ESI pos

### Step 2: 5-[(E)-Dimethylaminomethyleneamino]-1-(4-fluoro-1-methyl-indol-5-yl)imidazole-4-carbonyl chloride

A solution of 5-amino-1-(4-fluoro-1-methyl-indol-5-yl)imidazole-4-carboxylic acid (70 mg, 0.26 mmol, 1.0 eq), oxalyl chloride (26 μL, 0.31 mmol, 1.2 eq) and DMF (2 μL, 0.03 mmol, 0.1 eq) in CH₂Cl₂ (2 mL) was stirred at 25° C. for 1 h. The resulting solution was concentrated under reduced pressure to afford the title compound (70 mg, 0.2 mmol, 79% yield). MS: 344.1 [(M−Cl+OCH₃)+H]+, ESI pos (after MeOH quench)

### Step 3: tert-Butyl 4-[4-[2-chloro-4-[[5-[(E)-dimethylaminomethyleneamino]-1-(4-fluoro-1-methyl-indol-5-yl)imidazole-4-carbonyl]amino]benzoyl]piperazine-1-carbonyl]piperidine-1-carboxylate

To a solution of 5-[(E)-dimethylaminomethyleneamino]-1-(4-fluoro-1-methyl-indol-5-yl)imidazole-4-carbonyl chloride (45 mg, 0.13 mmol, 1.0 eq) and tert-butyl 4-[4-(4-amino-2-chlorobenzoyl)piperazine-1-carbonyl]piperidine-1-carboxylate (40 mg, 0.09 mmol, 0.7 eq) in pyridine (0.16 mL, 1.9 mmol, 15 eq) was stirred at 25° C. for 1 h. The resulting solution was concentrated under reduced pressure to the title compound (50 mg, 0.07 mmol, 51% yield). MS: 762.0 [M+H]+, ESI pos

### Step 4: tert-Butyl 4-[4-[4-[[5-amino-1-(4-fluoro-1-methyl-indol-5-yl)imidazole-4-carbonyl]amino]-2-chlorobenzoyl]piperazine-1-carbonyl]piperidine-1-carboxylate

A solution of tert-butyl 4-[4-[2-chloro-4-[[5-[(E)-dimethylaminomethyleneamino]-1-(4-fluoro-1-methyl-indol-5-yl)imidazole-4-carbonyl]amino]benzoyl]piperazine-1-carbonyl]piperidine-1-carboxylate (40 mg, 0.05 mmol, 1.0 eq) and NaOH (10 mg, 0.26 mmol, 5.0 eq) in MeOH (1 mL) and H₂O (0.250 mL) was stirred for 1 h at 60° C. The resulting solution was diluted with 50 ml of H₂O, then extracted with EtOAc (3×40 mL). The organic layers were combined, dried and concentrated under reduced pressure to give the title compound (30 mg, 0.04 mmol, 81% yield). MS: 707.3 [M+H]+, ESI pos

### Step 5: 5-amino-N-[3-chloro-4-[4-(piperidine-4-carbonyl)piperazine-1-carbonyl]phenyl]-1-(4-fluoro-1-methyl-indol-5-yl)imidazole-4-carboxamide;2,2,2-trifluoroacetic acid

To a solution of tert-butyl 4-[4-[4-[[5-amino-1-(4-fluoro-1-methyl-indol-5-yl)imidazole-4-carbonyl]amino]-2-chloro-benzoyl]piperazine-1-carbonyl]piperidine-1-carboxylate (30 mg, 0.04 mmol, 1.0 eq) and trifluoroacetic acid (1.0 mL) in DCM (3 mL) was stirred at 25° C. for 1 h. The resulting solution was concentrated under vacuum. The residue was purified by prep-HPLC (Gemini-C18 150×21.2 mm, 5 um, ACN— H₂O (0.1% TFA), 25-40) to give the title compound (28.4 mg). MS: 607.3 [M+H]+, ESI pos

### Compound 371

### 5-Amino-1-[2-(difluoromethyl)-3-fluoro-4-(fluoromethoxy)phenyl]-N-[3-fluoro-4-[4-[(2R)-morpholine-2-carbonyl]piperazine-1-carbonyl]phenyl]imidazole-4-carboxamide;2,2,2-trifluoroacetic acid

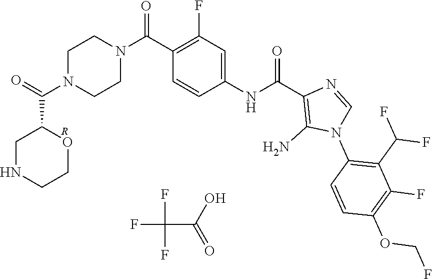

### Step 1: tert-Butyl(2R)-2-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]morpholine-4-carboxylate

2-(7-Azabenzotriazol-1-yl)-N,N,N′,N′-tetramethyluronium hexafluorophosphate (81 mg, 0.21 mmol, 1.5 eq) was added to a solution of 5-amino-1-[2-(difluoromethyl)-3-fluoro-4-(fluoromethoxy)phenyl]-N-[3-fluoro-4-(piperazine-1-carbonyl)phenyl]imidazole-4-carboxamide;hydrochloride (Intermediate 67, 80 mg, 0.14 mmol, 1.0 eq), (R)-4-(tert-butoxycarbonyl) morpholine-2-carboxylic acid (42 mg, 0.19 mmol, 1.3 eq) and NEt₃ (0.08 mL, 0.57 mmol, 4.0 eq) in CH₂Cl₂ (2 mL) in one portion, the mixture was stirred at 25° C. for 1 h. The reaction was concentrated to dryness and the residue was taken up in EtOAc (30 ml) and the organics washed with H₂O (2×30 ml) then 30 ml brine. The organics were then separated and dried (MgSO₄) before concentration to dryness. The crude was then purified by flash column chromatography eluting 0˜10% MeOH in CH₂Cl₂ to afford the title compound (90 mg, 0.12 mmol, 79% yield). MS: 738.0 [M+H]+, ESI pos

### Step 2: 5-Amino-1-[2-(difluoromethyl)-3-fluoro-4-(fluoromethoxy)phenyl]-N-[3-fluoro-4-[4-[(2R)-morpholine-2-carbonyl]piperazine-1-carbonyl]phenyl]imidazole-4-carboxamide;2,2,2-trifluoroacetic acid

A solution of tert-butyl(2R)-2-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]morpholine-4-carboxylate (90 mg, 0.12 mmol, 1.0 eq) in trifluoroacetic acid (1.0 mL, 0.12 mmol, 1.0 eq) the reaction was stirred at 25° C. for 1 h The reaction was concentrated to dryness. The crude material was purified by Prep-HPLC (Gemini-C18 150×21.2 mm, 5 um, MeCN— H₂O (0.1% TFA), 15-40% B) to give the title compound (60.1 mg, 0.08 mmol, 65% yield). MS: 638.0 [M+H]+, ESI pos

### Compound 372

### 5-Amino-N-[3-chloro-4-[4-(piperidine-4-carbonyl)piperazine-1-carbonyl]phenyl]-1-(1-cyclopropyl-4-fluoroindolin-5-yl)imidazole-4-carboxamide;formic acid

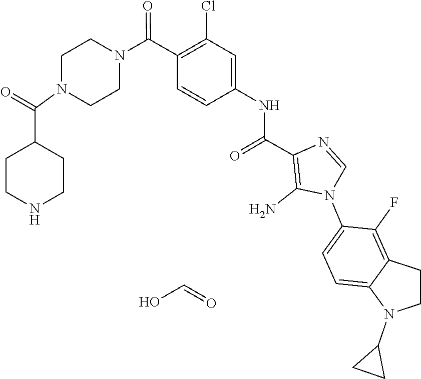 [SMILES: Nc1c(C(=O)Nc2ccc(C(=O)N3CCN(C(=O)C4CCNCC4)CC3)c(Cl)c2)ncn1-c1ccc2c(c1F)CCN2C1CC1 ; O=CO]

### Step 1: 5-Amino-1-(1-cyclopropyl-4-fluoroindolin-5-yl)imidazole-4-carboxylic acid

Ethyl 5-amino-1-(1-cyclopropyl-4-fluoroindolin-5-yl)imidazole-4-carboxylate (520 mg, 1.57 mmol, 1.0 eq) was dissolved in i-PrOH (10 mL). A solution of NaOH (314 mg, 7.87 mmol, 5.0 eq) in H₂O (5 mL) was added dropwise at 25° C. and stirred at 50° C. for 6 h. Solvent was evaporated. The residue was diluted with H₂O (10 mL), and the solution was subjected to extraction with EtOAc (2×10 mL). The aqueous layer was acidified to pH 5 with HCl (2 M), and the product was collected by filtration to give the title compound (310 mg, 1.03 mmol, 65% yield). MS: 303.0 [M+H]+

### Step 2: 5-Amino-1-(1-cyclopropyl-4-fluoroindolin-5-yl)imidazole-4-carbonyl chloride

A solution of 5-amino-1-(1-cyclopropyl-4-fluoroindolin-5-yl)imidazole-4-carboxylic acid (100.0 mg, 0.33 mmol, 1.0 eq), oxalyl chloride (83 mg, 0.66 mmol, 2.0 eq) and DMF (2 mg, 0.03 mmol, 0.1 eq) in CH₂Cl₂ (2 mL) under N₂ was stirred at 25° C. for 1 h. The resultant solution was evaporated to dryness and azeotroped with CH₂Cl₂ to give the title compound (100 mg, 0.31 mmol, 66% yield) which was used without further purification. MS: 317.1 [M−Cl+OCH₃+H]+ (after MeOH quench)

### Step 3: tert-Butyl 4-[4-[4-[[5-amino-1-(1-cyclopropyl-4-fluoroindolin-5-yl)imidazole-4-carbonyl]amino]-2-chlorobenzoyl]piperazine-1-carbonyl]piperidine-1-carboxylate

A solution of tert-butyl 4-[4-(4-amino-2-chlorobenzoyl)piperazine-1-carbonyl]piperidine-1-carboxylate (112 mg, 0.25 mmol, 0.8 eq) and pyridine (0.5 mL, 6.24 mmol, 20.0 eq) in CH₂Cl₂ (2 mL) was stirred at 25° C. This was followed by the addition of a solution of 5-amino-1-(1-cyclopropyl-4-fluoroindolin-5-yl)imidazole-4-carbonyl chloride (100 mg, 0.31 mmol, 1.0 eq) in CH₂Cl₂ (2 mL). The mixture was stirred at 25° C. for 1 h under N₂ before concentration under reduced pressure. The residue was applied on a silica gel column and eluted with CH₂Cl₂/MeOH (10:1) to give the title compound (90 mg, 0.12 mmol, 34% yield). MS: 735.2 [M+H]+

### Step 4: 5-Amino-N-[3-chloro-4-[4-(piperidine-4-carbonyl)piperazine-1-carbonyl]phenyl]-1-(1-cyclopropyl-4-fluoroindolin-5-yl)imidazole-4-carboxamide; formic acid

To a solution of tert-butyl 4-[4-[4-[[5-amino-1-(1-cyclopropyl-4-fluoroindolin-5-yl)imidazole-4-carbonyl]amino]-2-chlorobenzoyl]piperazine-1-carbonyl]piperidine-1-carboxylate (90 mg, 0.12 mmol, 1.0 eq) in MeOH (10 mL) was added oxalyl chloride (155 mg, 1.22 mmol, 10.0 eq). The resultant mixture was stirred at 25° C. for 6 before concentrating under reduced pressure. The residue was purified by prep-HPLC [Gemini-C18 150×21.2 mm, 5 μm, MeCN— H₂O (0.1% FA), 25-50% B] to give the title compound (32.1 mg, 0.05 mmol, 38% yield). MS: 635.1 [M+H]+

### Compound 373

### 5-amino-1-[2-(difluoromethyl)-3-fluoro-4-(fluoromethoxy)phenyl]-N-[3-fluoro-4-[4-[(3R)-3-fluoropyrrolidine-3-carbonyl]piperazine-1-carbonyl]phenyl]imidazole-4-carboxamide;2,2,2-trifluoroacetic acid

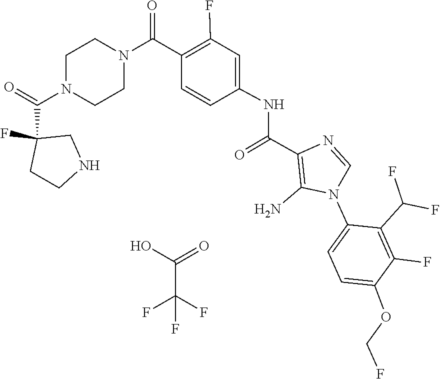 [SMILES: Nc1c(C(=O)Nc2ccc(C(=O)N3CCN(C(=O)[C@@]4(F)CCNC4)CC3)c(F)c2)ncn1-c1ccc(OCF)c(F)c1C(F)F ; O=C(O)C(F)(F)F]

### Step 1:(3R)-1-tert-butoxycarbonyl-3-fluoro-pyrrolidine-3-carboxylic acid

To a solution of sodium hydroxide (64.7 mg, 1.62 mmol, 5.0 eq) was added to O1-tert-butyl O₃-methyl (3R)-3-fluoropyrrolidine-1,3-dicarboxylate ([1438852-70-0], 80 mg, 0.32 mmol, 1.0 eq) in IPA (4 mL) and water (2 mL), the resultant mixture was stirred at 50° C. for 2. The mixture was adjusted to pH 5-6 with 2N aqueous HCl and extracted 2× with EtOAc. The combined organic layer was dried over anhydrous sodium sulfate and concentrated under vacuum afford the title compound (50 mg, 0.21 mmol, 66.26% yield). MS: 232.1 [M−H]−

### Step 2: tert-butyl(3R)-3-[4-[4-[[5-amino-1-[2-(difluoromethyl)-3-fluoro-4-(fluoromethoxy)phenyl]imidazole-4-carbonyl]amino]-2-fluoro-benzoyl]piperazine-1-carbonyl]-3-fluoro-pyrrolidine-1-carboxylate

To a solution of 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (50 mg, 0.1 mmol, 1.0 eq) and (3R)-1-tert-butoxycarbonyl-3-fluoro-pyrrolidine-3-carboxylic acid (22 mg, 0.1 mmol, 1.0 eq) in DCM (1 mL) was added 2-(7-azabenzotriazol-1-yl)-N,N,N′,N′-tetramethyluronium hexafluorophosphate (72.5 mg, 0.19 mmol, 2.0 eq) and triethylamine (0.04 mL, 0.29 mmol, 3.0 eq) and the mixture stirred at 25° C. for 2 h. The reaction was quenched with water (25 mL). The resulting solution was extracted with ethyl acetate (20 mL×3). The combined organic phases were dried over anhydrous sodium sulfate and concentrated under reduced pressure. The residue was purified by silica gel chromatography to afford the title compound (45 mg, 0.06 mmol, 63.81% yield). MS: 640.0 [M+H−Boc]+

### Step 3: 5-amino-1-[2-(difluoromethyl)-3-fluoro-4-(fluoromethoxy)phenyl]-N-[3-fluoro-4-[4-[(3R)-3-fluoropyrrolidine-3-carbonyl]piperazine-1-carbonyl]phenyl]imidazole-4-carboxamide;2,2,2-trifluoroacetic acid

A solution of tert-butyl(3R)-3-[4-[4-[[5-amino-1-[2-(difluoromethyl)-3-fluoro-4-(fluoromethoxy)phenyl]imidazole-4-carbonyl]amino]-2-fluoro-benzoyl]piperazine-1-carbonyl]-3-fluoro-pyrrolidine-1-carboxylate (31 mg, 0.04 mmol, 1.0 eq) and trifluoroacetic acid (1.0 mL, 12.98 mmol, 306.41 eq) in DCM (3 mL) was stirred at 25° C. for 1 h. The resulting solution was concentrated under vacuum. The residue was purified by Prep-HPLC (-Gemini-C18 150×21.2 mm, 5 um, ACN— H₂O (0.1% TFA), 25-40) to afford the title compound (20 mg, 0.03 mmol, 61.94% yield). MS: 640.1 [M+H]+

### Compound 374

### 5-amino-1-[2-(difluoromethyl)-3-fluoro-4-(fluoromethoxy)phenyl]-N-[3-fluoro-4-[4-[(3S)-3-fluoropyrrolidine-3-carbonyl]piperazine-1-carbonyl]phenyl]imidazole-4-carboxamide;2,2,2-trifluoroacetic acid

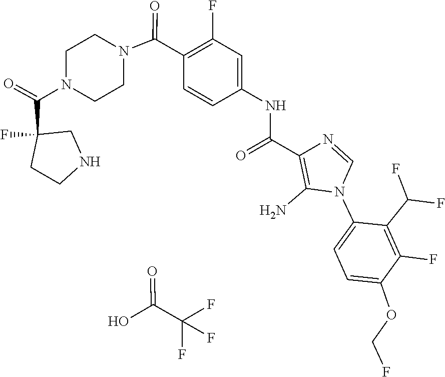 [SMILES: Nc1c(C(=O)Nc2ccc(C(=O)N3CCN(C(=O)[C@]4(F)CCNC4)CC3)c(F)c2)ncn1-c1ccc(OCF)c(F)c1C(F)F ; O=C(O)C(F)(F)F]

### Step 1:(3S)-1-tert-butoxycarbonyl-3-fluoro-pyrrolidine-3-carboxylic acid

To a solution of lithium hydroxide (0.02 mL, 1.62 mmol, 5.0 eq) was added to O1-tert-butyl O3-methyl (3S)-3-fluoropyrrolidine-1,3-dicarboxylate ([1438852-71-1], 80 mg, 0.32 mmol, 1.0 eq) in methanol (12 mL) and water (6 mL), the resultant mixture was stirred at 25° C. for 2 h. The resulting solution was concentrated under vacuum at 30° C. the mixture was extracted using water and ethyl acetate. The pH of the aq. layer was acidified, filtered and the material was lyophilized to afford the title compound (53 mg, 0.23 mmol, 70.24% yield) as a colorless liquid. MS: 232.1 [M−H]−

### Step 2: tert-butyl(3S)-3-[4-[4-[[5-amino-1-[2-(difluoromethyl)-3-fluoro-4-(fluoromethoxy)phenyl]imidazole-4-carbonyl]amino]-2-fluoro-benzoyl]piperazine-1-carbonyl]-3-fluoro-pyrrolidine-1-carboxylate

To a solution of (3S)-1-tert-butoxycarbonyl-3-fluoro-pyrrolidine-3-carboxylic acid (43 mg, 0.18 mmol, 1.0 eq), 2-(7-azabenzotriazol-1-yl)-N,N,N′,N′-tetramethyluronium hexafluorophosphate (140.2 mg, 0.37 mmol, 2.0 eq) and triethylamine (0.08 mL, 0.55 mmol, 3.0 eq) in DMF (5 mL) was added 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (Intermediate 67, 48 mg, 0.09 mmol, 0.5 eq) and the reaction was stirred at 25° C. for 2 h. The reaction was concentrated to dryness and the residue was taken up in EtOAc (20 ml) and the organics washed with 2×20 ml water then 1×20 ml brine. The combined organic layers were dried (MgSO₄) before concentration to dryness. The residue was purified by silica gel chromatography to afford the tile compound (63 mg, 0.09 mmol, 46.2% yield). MS: 640.1 [M−Boc+H]+

### Step 3: 5-amino-1-[2-(difluoromethyl)-3-fluoro-4-(fluoromethoxy)phenyl]-N-[3-fluoro-4-[4-[(3S)-3-fluoropyrrolidine-3-carbonyl]piperazine-1-carbonyl]phenyl]imidazole-4-carboxamide;2,2,2-trifluoroacetic acid

A solution of tert-butyl(3S)-3-[4-[4-[[5-amino-1-[2-(difluoromethyl)-3-fluoro-4-(fluoromethoxy)phenyl]imidazole-4-carbonyl]amino]-2-fluoro-benzoyl]piperazine-1-carbonyl]-3-fluoro-pyrrolidine-1-carboxylate (63 mg, 0.09 mmol, 1.0 eq) and trifluoroacetic acid (194 mg, 1.7 mmol, 20.0 eq) in DCM (2 mL) was stirred at 25° C. for 1 h. The resulting solution was concentrated under vacuum. The crude product was purified by Prep-HPLC (Gemini-C18 150×21.2 mm, 5 um, ACN— H₂O (0.1% TFA), 15-45) to afford the title compound (46.5 mg, 0.06 mmol, 72.23% yield) as a freeze-dried solid. MS: 640.1 [M+H]+

### Compound 375

### 5-Amino-1-[4-(cyclopropyloxy)-2,3-difluorophenyl]-N-[3-fluoro-4-[4-(piperazine-1-carbonyl)piperazine-1-carbonyl]phenyl]imidazole-4-carboxamide;2,2,2-trifluoroacetic acid

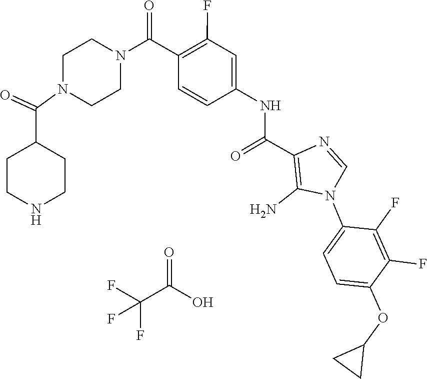 [SMILES: Nc1c(C(=O)Nc2ccc(C(=O)N3CCN(C(=O)C4CCNCC4)CC3)c(F)c2)ncn1-c1ccc(OC2CC2)c(F)c1F ; O=C(O)C(F)(F)F]

### Step 1: tert-Butyl 4-[4-[4-[[5-amino-1-[4-(cyclopropyloxy)-2,3-difluorophenyl]imidazole-4-carbonyl]amino]-2-fluorobenzoyl]piperazine-1-carbonyl]piperazine-1-carboxylate

To a solution of tert-butyl 4-[4-(4-amino-2-fluorobenzoyl)piperazine-1-carbonyl]piperazine-1-carboxylate (87 mg, 0.2 mmol, 0.7 eq) and pyridine (453 mg, 5.74 mmol, 20.0 eq) in CH₂Cl₂ (5 mL) was added a suspension of 5-amino-1-[4-(cyclopropoxy)-2,3-difluoro-phenyl]imidazole-4-carbonyl chloride (90 mg, 0.29 mmol, 1.0 eq) in CH₂Cl₂ (5 mL) at 25° C. The mixture was stirred at 25° C. for 12 h under N₂. The resulting solution was evaporated under reduced pressure. The residue was purified by column chromatography (silica gel, 0 to 10% MeOH in CH₂Cl₂) to give the title compound (110 mg, 0.15 mmol, 54% yield). MS: 712.8 [M+H]+, ESI pos

### Step 2: 5-Amino-1-[4-(cyclopropyloxy)-2,3-difluorophenyl]-N-[3-fluoro-4-[4-(piperazine-1-carbonyl)piperazine-1-carbonyl]phenyl]imidazole-4-carboxamide;2,2,2-trifluoroacetic acid

To a solution of tert-butyl 4-[4-[4-[[5-amino-1-[4-(cyclopropyloxy)-2,3-difluorophenyl]imidazole-4-carbonyl]amino]-2-fluorobenzoyl]piperazine-1-carbonyl]piperazine-1-carboxylate (80 mg, 0.11 mmol, 1.0 eq) in CH₂Cl₂ (3 mL) was added trifluoroacetic acid (1.0 mL, 0.11 mmol, 1.0 eq). The reaction was stirred at 25° C. for 1 h and then concentrated to dryness to give a crude product. The crude was purified by Prep-HPLC (Gemini-C18 150×21.2 mm, 5 μm, MeCN— H₂O (0.1% TFA), 15-40% B) to give the title compound (35.6 mg, 0.05 mmol, 43% yield). MS 613.0 [M+H]+

### Compound 376

### 5-Amino-N-[4-[4-[(1R,5S,6r)-3-azabicyclo[3.1.0]hexane-6-carbonyl]piperazine-1-carbonyl]-3-chlorophenyl]-1-[2-chloro-4-(fluoromethoxy)phenyl]imidazole-4-carboxamide;2,2,2-trifluoroacetic acid

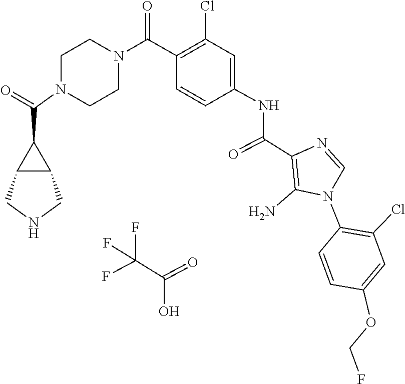 [SMILES: Nc1c(C(=O)Nc2ccc(C(=O)N3CCN(C(=O)[C@H]4[C@@H]5CNC[C@@H]54)CC3)c(Cl)c2)ncn1-c1ccc(OCF)cc1Cl ; O=C(O)C(F)(F)F]

### Step 1: 5-Amino-1-[2-chloro-4-(fluoromethoxy)phenyl]imidazole-4-carbonyl chloride

To 5-amino-1-[2-chloro-4-(fluoromethoxy)phenyl]imidazole-4-carboxylic acid (100 mg, 0.35 mmol, 1.0 eq) under N₂ was added thionyl chloride (2.0 mL, 28 mmol, 79 eq) at 25° C. The solution was stirred at 25° C. for 1 h under N₂. The resultant solution was evaporated to dryness and azeotroped with CH₂Cl₂ to afford the title compound (100 mg, 0.33 mmol, 94% yield) which was used without further purification. MS: 300.0 [M−Cl+OCH₃+H]+ (after MeOH quench).

### Step 2: tert-Butyl (1R,5S,6r)-6-[4-[4-[[5-amino-1-[2-chloro-4-(fluoromethoxy)phenyl]imidazole-4-carbonyl]amino]-2-chlorobenzoyl]piperazine-1-carbonyl]-3-azabicyclo[3.1.0]hexane-3-carboxylate

A solution of tert-butyl(1R,5S,6r)-6-[4-(4-amino-2-chlorobenzoyl)piperazine-1-carbonyl]-3-azabicyclo[3.1.0]hexane-3-carboxylate (Intermediate 22, 118 mg, 0.26 mmol, 0.8 eq) and pyridine (520 mg, 6.58 mmol, 20.0 eq) in CH₂Cl₂ (2 mL) was stirred at 25° C. This was followed by the addition of a solution of 5-amino-1-[2-chloro-4-(fluoromethoxy)phenyl]imidazole-4-carbonyl chloride (100.0 mg, 0.33 mmol, 1.0 eq) in CH₂Cl₂ (2 mL). The mixture was stirred at 25° C. for 1 h under N₂. The resulting solution was concentrated under reduced pressure. The residue was applied on a silica gel column and eluted with CH₂Cl₂/MeOH (10:1) to give the title compound (120 mg, 0.17 mmol, 46% yield). MS: 660.0 [M−t−Bu+H]+

### Step 3: 5-Amino-N-[4-[4-[(1R,5S,6r)-3-azabicyclo[3.1.0]hexane-6-carbonyl]piperazine-1-carbonyl]-3-chlorophenyl]-1-[2-chloro-4-(fluoromethoxy)phenyl]imidazole-4-carboxamide;2,2,2-trifluoroacetic acid

A solution of tert-butyl(1R,5S,6r)-6-[4-[4-[[5-amino-1-[2-chloro-4-(fluoromethoxy)phenyl]imidazole-4-carbonyl]amino]-2-chlorobenzoyl]piperazine-1-carbonyl]-3-azabicyclo[3.1.0]hexane-3-carboxylate (120 mg, 0.17 mmol, 1.0 eq), trifluoroacetic acid (1.0 mL, 13 mmol, 78 eq) in CH₂Cl₂ (4 mL) was stirred at 25° C. for 1 h. The resulting solution was concentrated under reduced pressure. The crude product was purified by Prep-HPLC (Gemini-C18 150×21.2 mm, 5 μm, MeCN— H₂O (0.1% TFA), 30-60% B) to get the title compound (56.6 mg, 0.08 mmol, 46% yield). MS: 616.0 [M+H]+

### Compound 377

### 5-Amino-1-[4-(cyclopropyloxy)-2-(difluoromethyl)-3-fluorophenyl]-N-[3-fluoro-4-[4-[(3aS,6aR)-2,3,3a,4,6,6a-hexahydro-1H-pyrrolo[3,4-c]pyrrole-5-carbonyl]piperazine-1-carbonyl]phenyl]imidazole-4-carboxamide;2,2,2-trifluoroacetic acid

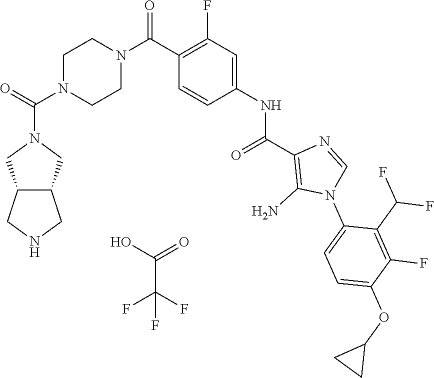 [SMILES: Nc1c(C(=O)Nc2ccc(C(=O)N3CCN(C(=O)N4C[C@H]5CNC[C@H]5C4)CC3)c(F)c2)ncn1-c1ccc(OC2CC2)c(F)c1C(F)F ; O=C(O)C(F)(F)F]

### Step 1: Ethyl 5-amino-1-[4-(cyclopropyloxy)-2-(difluoromethyl)-3-fluorophenyl]imidazole-4-carboxylate

To a solution of ethyl 2-amino-2-cyanoacetate; 4-methylbenzene-1-sulphonic aci (4148 mg, 13.81 mmol, 2.0 eq) in MeCN (2 mL) were added NEt₃ (3.85 mL, 27.6 mmol, 4.0 eq) and triethyl orthoformate (30.4 mL, 183 mmol, 26.5 eq), the resultant mixture was stirred at 80° C. for 2 h, the solvent was removed under reduced pressure and the residue was diluted with MeCN (2 mL), then 4-(cyclopropoxy)-2-(difluoromethyl)-3-fluoro-aniline (Intermediate 35, 1500 mg, 6.91 mmol, 1.0 eq) was added and the resultant mixture was stirred at 50° C. for 15 h. The mixture was concentrated under reduced pressure and the residue was purified by silica gel chromatography (PE:EtOAc=30:70) to afford the title compound (1800 mg, 5.07 mmol, 73% yield). MS: 356.1 [M+H]+

### Step 2: 5-Amino-1-[4-(cyclopropyloxy)-2-(difluoromethyl)-3-fluorophenyl]imidazole-4-carboxylic acid

To a solution of NaOH (1013.1 mg, 25.33 mmol, 5.0 eq) was added to ethyl 5-amino-1-[4-(cyclopropyloxy)-2-(difluoromethyl)-3-fluorophenyl]imidazole-4-carboxylate (1800 mg, 5.07 mmol, 1.0 eq) in i-PrOH (14 mL) and H₂O (7 mL). The resultant mixture was stirred at 50° C. for 2 h. The mixture was adjusted to pH 5-6 with 2N aqueous HCl and extracted with EtOAc. The reaction mixture was partitioned between EtOAc and H₂O. The aqueous layer was extracted again with EtOAc. The combined organic layer was dried over Na₂SO₄ and concentrated under reduced pressure give the title compound (720 mg, 2.2 mmol, 43% yield). MS: 328.0 [M+H]+

### Step 3: 5-Amino-1-[4-(cyclopropyloxy)-2-(difluoromethyl)-3-fluorophenyl]imidazole-4-carbonyl chloride

To 5-amino-1-[4-(cyclopropyloxy)-2-(difluoromethyl)-3-fluorophenyl]imidazole-4-carboxylic acid (100 mg, 0.31 mmol, 1.0 eq) under N₂ was added thionyl chloride (1.75 mL, 24.1 mmol, 78.8 eq) at 25° C. The solution was stirred at 25° C. for 1 h under N₂, evaporated to dryness, and azeotroped with CH₂Cl₂ to afford the title compound (100 mg, 0.29 mmol, 95% yield) which was used without further purification. MS: 342.0 [M−Cl+OCH₃+H]+ (after MeOH quench)

### Step 4: tert-Butyl (3aR,6aS)-2-[4-[4-[[5-amino-1-[4-(cyclopropyloxy)-2-(difluoromethyl)-3-fluorophenyl]imidazole-4-carbonyl]amino]-2-fluorobenzoyl]piperazine-1-carbonyl]-1,3,3a,4,6,6a-hexahydropyrrolo[3,4-c]pyrrole-5-carboxylate

To a solution of tert-butyl(3aR,6aS)-2-[4-(4-amino-2-fluorobenzoyl)piperazine-1-carbonyl]-1,3,3a,4,6,6a-hexahydropyrrolo[3,4-c]pyrrole-5-carboxylate (53.4 mg, 0.12 mmol, 0.8 eq) and pyridine (0.18 mL, 2.17 mmol, 15.0 eq) in CH₂Cl₂ (30 mL) was added a suspension of 5-amino-1-[4-(cyclopropyloxy)-2-(difluoromethyl)-3-fluorophenyl]imidazole-4-carbonyl chloride (50 mg, 0.14 mmol, 1.0 eq) in CH₂Cl₂ (30 mL) at 25° C. The mixture was stirred at 25° C. for 1 h under N₂. The resulting solution was evaporated under reduced pressure. The residue was purified by column chromatography (silica gel, 0 to 10% MeOH in CH₂Cl₂) to give the title compound (70 mg, 0.09 mmol, 63% yield). MS: 671.1 [M+H−Boc]+

### Step 5: 5-Amino-1-[4-(cyclopropyloxy)-2-(difluoromethyl)-3-fluorophenyl]-N-[3-fluoro-4-[4-[(3aS,6aR)-2,3,3a,4,6,6a-hexahydro-1H-pyrrolo[3,4-c]pyrrole-5-carbonyl]piperazine-1-carbonyl]phenyl]imidazole-4-carboxamide;2,2,2-trifluoroacetic acid

To a solution of tert-butyl(3aR,6aS)-2-[4-[4-[[5-amino-1-[4-(cyclopropyloxy)-2-(difluoromethyl)-3-fluorophenyl]imidazole-4-carbonyl]amino]-2-fluorobenzoyl]piperazine-1-carbonyl]-1,3,3a,4,6,6a-hexahydropyrrolo[3,4-c]pyrrole-5-carboxylate (40 mg, 0.05 mmol, 1.0 eq) and trifluoroacetic acid (0.99 mL, 13 mmol, 250 eq) in CH₂Cl₂ (3 mL) was stirred at 25° C. for 1 h. The resulting solution was concentrated under reduced pressure. The residue was purified by Prep-HPLC (Gemini-C18 150×21.2 mm, 5 μm, MeCN— H₂O (0.1% TFA), 25-40% B) to afford the title compound (16.5 mg, 0.02 mmol, 38% yield). MS: 671.1 [M+H]+, ESI pos

### Compound 378

### 5-Amino-1-[4-(cyclopropyloxy)-2,3-difluorophenyl]-N-[4-[4-(2,6-diazaspiro[3.3]heptane-2-carbonyl)piperazine-1-carbonyl]-3-fluorophenyl]imidazole-4-carboxamide;2,2,2-trifluoroacetic acid

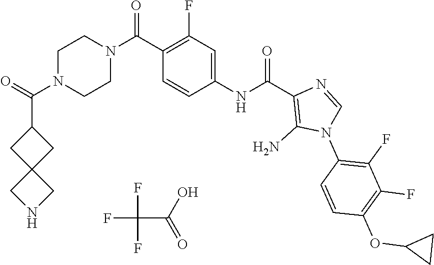 [SMILES: Nc1c(C(=O)Nc2ccc(C(=O)N3CCN(C(=O)C4CC5(CNC5)C4)CC3)c(F)c2)ncn1-c1ccc(OC2CC2)c(F)c1F ; O=C(O)C(F)(F)F]

### Step 1: tert-Butyl 6-[4-(2-fluoro-4-nitrobenzoyl)piperazine-1-carbonyl]-2,6-diazaspiro[3.3]heptane-2-carboxylate

To a solution of (2-fluoro-4-nitrophenyl)-piperazin-1-yl-methanone;hydrochloride ([1593114-33-0], 478.6 mg, 1.65 mmol, 1.0 eq) in CH₂Cl₂ (10 mL) was added Triphosgene (294.41 mg, 0.99 mmol, 0.6 eq) at 25° C. After stirring for 0.5 h, NEt₃ (1668 mg, 16.52 mmol, 10.0 eq) and tert-butyl 2,6-diazaspiro[3.3]heptane-2-carboxylate;oxalic acid (401 mg, 0.83 mmol, 0.5 eq) was added, and the resulting mixture was stirred for 1.5 h at 25° C. The resulting solution was concentrated under reduced pressure. The residue was applied on a silica gel column and eluted with MeOH/CH₂Cl₂ (1/20) to give the title compound (70% yield). MS: 422.1 [M+H−tBu]+, ESI pos

### Step 2: tert-Butyl 6-[4-(4-amino-2-fluorobenzoyl)piperazine-1-carbonyl]-2,6-diazaspiro[3.3]heptane-2-carboxylate

A mixture of tert-butyl 6-[4-(2-fluoro-4-nitrobenzoyl)piperazine-1-carbonyl]-2,6-diazaspiro[3.3]heptane-2-carboxylate (500 mg, 1.05 mmol, 1.0 eq), Fe dust (586.4 mg, 10.47 mmol, 10.0 eq), and NH₄Cl (560 mg, 10.47 mmol, 10.0 eq), in EtOH (10 mL) and H₂O (3 mL) was stirred at 50° C. for 1 h. The mixture was filtered, and the filtrate was concentrated under reduced pressure. The residue was diluted with H₂O and extracted with 2×50 mL of EtOAc. The organic layers were combined, washed with brine, dried and concentrated under reduced pressure to the title compound (162 mg, 0.36 mmol, 32% yield).

MS: 448 [M+H]⁺, ESI pos

### Step 3: tert-Butyl 6-[4-[4-[[5-amino-1-[4-(cyclopropyloxy)-2,3-difluorophenyl]imidazole-4-carbonyl]amino]-2-fluorobenzoyl]piperazine-1-carbonyl]-2,6-diazaspiro[3.3]heptane-2-carboxylate

To a solution of tert-butyl 6-[4-(4-amino-2-fluorobenzoyl)piperazine-1-carbonyl]-2,6-diazaspiro[3.3]heptane-2-carboxylate (39 mg, 0.09 mmol, 0.7 eq) and pyridine (0.21 mL, 2.55 mmol, 20.0 eq) was added a suspension of 5-amino-1-[4-(cyclopropoxy)-2,3-difluoro-phenyl]imidazole-4-carbonyl chloride (40 mg, 0.13 mmol, 1.0 eq) at 25° C. The mixture was stirred at 25° C. for 1 h under N₂. The resulting solution was evaporated under reduced pressure. The residue was purified by column chromatography (silica gel, 0 to 10% MeOH in CH₂Cl₂) to afford the title compound (43 mg, 0.06 mmol, 46% yield). MS: 725.7 [M+H]⁺, ESI pos

### Step 4: 5-Amino-1-[4-(cyclopropyloxy)-2,3-difluorophenyl]-N-[4-[4-(2,6-diazaspiro[3.3]heptane-2-carbonyl)piperazine-1-carbonyl]-3-fluorophenyl]imidazole-4-carboxamide;2,2,2-trifluoroacetic acid

A solution of tert-butyl 6-[4-[4-[[5-amino-1-[4-(cyclopropyloxy)-2,3-difluorophenyl]imidazole-4-carbonyl]amino]-2-fluorobenzoyl]piperazine-1-carbonyl]-2,6-diazaspiro[3.3]heptane-2-carboxylate (33 mg, 0.05 mmol, 1.0 eq) and trifluoroacetic acid (5 mg, 0.05 mmol, 1.0 eq) in CH₂Cl₂ (1 mL) was stirred at 25° C. for 1 h. The resulting solution was concentrated under reduced pressure. The crude product was purified by Prep-HPLC (Gemini-C18 150×21.2 mm, 5 μm, MeCN— H₂O (0.1% TFA), 15-45% B) to the title compound (28.2 mg, 0.04 mmol, 77% yield). MS: 626.0 [M+H]+, ESI pos

### Compound 379

### 5-Amino-N-[3-chloro-4-[4-[(1S,5R,6r)-6-fluoro-3-azabicyclo[3.1.0]hexane-6-carbonyl]piperazine-1-carbonyl]phenyl]-1-[2-chloro-4-(fluoromethoxy)phenyl]imidazole-4-carboxamide;2,2,2-trifluoroacetic acid

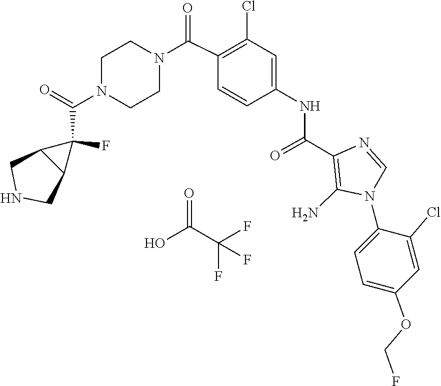 [SMILES: Nc1c(C(=O)Nc2ccc(C(=O)N3CCN(C(=O)[C@@]4(F)[C@@H]5CNC[C@@H]54)CC3)c(Cl)c2)ncn1-c1ccc(OCF)cc1Cl ; O=C(O)C(F)(F)F]

### Step 1: tert-butyl(1S,5R,6r)-6-(4-(4-(5-amino-1-(2-chloro-4-(fluoromethoxy)phenyl)-1H-imidazole-4-carboxamido)-2-chlorobenzoyl)piperazine-1-carbonyl)-6-fluoro-3-azabicyclo[3.1.0]hexane-3-carboxylate

To a solution of 5-amino-1-[2-chloro-4-(fluoromethoxy)phenyl]-N-[3-chloro-4-(piperazine-1-carbonyl)phenyl]imidazole-4-carboxamide (70 mg, 0.14 mmol, 1.0 eq) in CH₂Cl₂ (3 mL) were added (1S,5R,6r)-3-tert-butoxycarbonyl-6-fluoro-3-azabicyclo[3.1.0]hexane-6-carboxylic acid (67 mg, 0.28 mmol, 2.0 eq), 1,3,5,2,4,6-trioxatriphosphorinane, 2,4,6-tributyl-, 2,4,6-trioxide (104 mg, 0.28 mmol, 2.0 eq) and N,N-diisopropylethylamine (53 mg, 0.41 mmol, 3.0 eq). The resultant mixture was stirred at 25° C. for 1 h, and then the solvent was removed under reduced pressure. The residue was diluted with EtOAc (5 mL) and washed with H₂O (4×5 mL), dried over Na₂SO₄, and concentrated under reduced pressure to give the title compound (90 mg, 0.12 mmol, 59% yield). MS: 678.0 [M−t−Bu+H]⁺, ESI pos

### Step 2: 5-Amino-N-[3-chloro-4-[4-[(1S,5R,6r)-6-fluoro-3-azabicyclo[3.1.0]hexane-6-carbonyl]piperazine-1-carbonyl]phenyl]-1-[2-chloro-4-(fluoromethoxy)phenyl]imidazole-4-carboxamide;2,2,2-trifluoroacetic acid

A solution of tert-butyl(1S,5R,6)-6-[4-[4-[[5-amino-1-[2-chloro-4-(fluoromethoxy)phenyl]imidazole-4-carbonyl]amino]-2-chlorobenzoyl]piperazine-1-carbonyl]-6-fluoro-3-azabicyclo[3.1.0]hexane-3-carboxylate (90 mg, 0.12 mmol, 1.0 eq), trifluoroacetic acid (1.0 mL, 13 mmol, 100 eq) in CH₂Cl₂ (4 mL) was stirred at 25° C. for 1 h. The resulting solution was concentrated under reduced pressure. The crude product was purified by Prep-HPLC (Gemini-C18 150×21.2 mm, 5 μm, MeCN— H₂O (0.1% TFA), 30-60% B) to afford the title compound (62.1 mg, 0.08 mmol, 68% yield). MS: 634.0 [M+H]+; ESI pos

### Compound 380

### 5-Amino-N-[3-chloro-4-[4-(2,6-diazaspiro[3.3]heptane-2-carbonyl)piperazine-1-carbonyl]phenyl]-1-[4-(cyclopropyloxy)-2-(difluoromethyl)-3-fluorophenyl]imidazole-4-carboxamide;2,2,2-trifluoroacetic acid

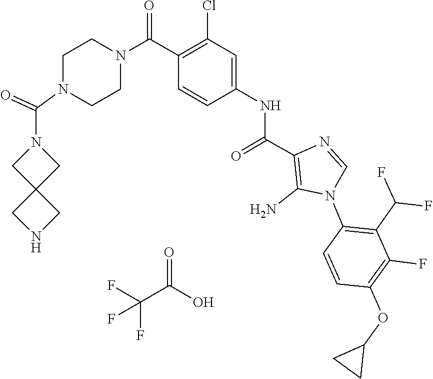 [SMILES: Nc1c(C(=O)Nc2ccc(C(=O)N3CCN(C(=O)N4CC5(CNC5)C4)CC3)c(Cl)c2)ncn1-c1ccc(OC2CC2)c(F)c1C(F)F ; O=C(O)C(F)(F)F]

### Step 1: tert-Butyl 6-[4-(2-chloro-4-nitrobenzoyl)piperazine-1-carbonyl]-2,6-diazaspiro[3.3]heptane-2-carboxylate

A solution of (2-chloro-4-nitro-phenyl)-piperazin-1-yl-methanone;hydrochloride ([1605138-92-8], 300 mg, 0.98 mmol, 1.0 eq) and triphosgene (174 mg, 0.59 mmol, 0.6 eq) in CH₂Cl₂ (5 mL) was stirred at 25° C. for 0.3 h. tert-Butyl 2,6-diazaspiro[3.3]heptane-2-carboxylate; oxalic acid (286 mg, 0.59 mmol, 0.6 eq) and NEt₃ (1.37 mL, 9.8 mmol, 10.0 eq) were then added and the mixture was stirred at 25° C. for 1 h. The reaction was concentrated to dryness and the residue was taken up in EtOAc (10 ml) and washed with H₂O (2×3 ml) then 3 ml brine. The organics were then separated and dried (Na₂SO₄) before concentration to dryness. The crude was then purified by flash column chromatography eluting 60% EtOAc in PE. The desired fractions were concentrated to dryness under reduced pressure to give the title compound (310 mg, 0.63 mmol, 64% yield). MS: 517.0 [M+Na+H]+, ESI pos

### Step 2: tert-Butyl 6-[4-(4-amino-2-chlorobenzoyl)piperazine-1-carbonyl]-2,6-diazaspiro[3.3]heptane-2-carboxylate

A solution of tert-butyl 6-[4-(2-chloro-4-nitrobenzoyl)piperazine-1-carbonyl]-2,6-diazaspiro[3.3]heptane-2-carboxylate (240 mg, 0.49 mmol, 1.0 eq), Fe powder (272.1 mg, 4.86 mmol, 10.0 eq) and NH₄Cl (262 mg, 4.86 mmol, 10.0 eq) in EtOH (3 mL) and H₂O (1 mL) was stirred at 70° C. for 1 h. The solid was filtered. The filtrate was concentrated under reduced pressure. The residue was diluted with 40 mL of EtOAc. The organic layer was washed with H₂O (3×20 mL), dried and concentrated under reduced pressure to give the title compound (170 mg, 0.37 mmol, 75% yield). MS: 464.1 [M+H]+, ESI pos

### Step 3: tert-Butyl 6-[4-[4-[[5-amino-1-[4-(cyclopropyloxy)-2-(difluoromethyl)-3-fluorophenyl]imidazole-4-carbonyl]amino]-2-chlorobenzoyl]piperazine-1-carbonyl]-2,6-diazaspiro[3.3]heptane-2-carboxylate

To a solution of tert-butyl 6-[4-(4-amino-2-chlorobenzoyl)piperazine-1-carbonyl]-2,6-diazaspiro[3.3]heptane-2-carboxylate (51 mg, 0.11 mmol, 0.7 eq) in CH₂Cl₂ (3 mL) was added pyridine (252 mg, 3.19 mmol, 20.0 eq) and 5-amino-1-[4-(cyclopropyloxy)-2-(difluoromethyl)-3-fluorophenyl]imidazole-4-carbonyl chloride (60 mg, 0.16 mmol, 1.0 eq). The reaction was stirred at 25° C. for 16 h and then concentrated to dryness. The residue was taken up in CH₂Cl₂ (20 mL) and the organics washed with H₂O (2×20 mL) then brine (20 mL). The organics were then separated and dried (MgSO₄) before concentrating to dryness. The crude was then purified by flash column chromatography eluting 12% MeOH in CH₂Cl₂. The desired fractions were concentrated to dryness under reduced pressure to afford the title compound (80 mg, 0.1 mmol, 64% yield). MS 773.1 [M+H]+, ESI pos

### Step 4: 5-Amino-N-[3-chloro-4-[4-(2,6-diazaspiro[3.3]heptane-2-carbonyl)piperazine-1-carbonyl]phenyl]-1-[4-(cyclopropyloxy)-2-(difluoromethyl)-3-fluorophenyl]imidazole-4-carboxamide;2,2,2-trifluoroacetic acid

To a solution of tert-butyl 6-[4-[4-[[5-amino-1-[4-(cyclopropyloxy)-2-(difluoromethyl)-3-fluorophenyl]imidazole-4-carbonyl]amino]-2-chlorobenzoyl]piperazine-1-carbonyl]-2,6-diazaspiro[3.3]heptane-2-carboxylate (70 mg, 0.09 mmol, 1.0 eq) in CH₂Cl₂ (3 mL) was added trifluoroacetic acid (1.0 mL, 13 mmol, 1.5 eq). The resultant mixture was stirred at 25° C. for 1 h. The solvent was removed under reduced pressure and the residue was purified by prep-HPLC [Gemini-C18, 150×21.2 mm, 5 μm, MeCN-H₂O (0.1% TFA), 15-40% B] to afford the title compound (27 mg, 0.03 mmol, 38% yield). MS: 673.0 [M+H]+, ESI pos

### Compound 381

### 5-Amino-1-[4-(cyclopropyloxy)-2-(difluoromethyl)-3-fluorophenyl]-N-[3-fluoro-4-[4-(piperazine-1-carbonyl)piperazine-1-carbonyl]phenyl]imidazole-4-carboxamide;2,2,2-trifluoroacetic acid

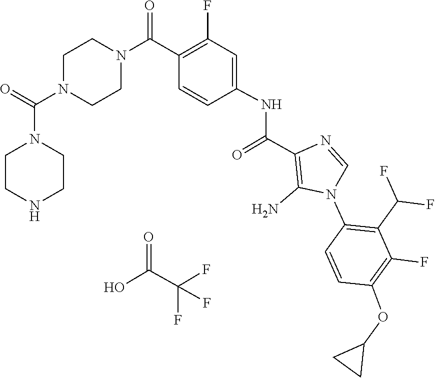 [SMILES: Nc1c(C(=O)Nc2ccc(C(=O)N3CCN(C(=O)N4CCNCC4)CC3)c(F)c2)ncn1-c1ccc(OC2CC2)c(F)c1C(F)F ; O=C(O)C(F)(F)F]

### Step 1: tert-Butyl 4-[4-[4-[[5-amino-1-[4-(cyclopropyloxy)-2-(difluoromethyl)-3-fluorophenyl]imidazole-4-carbonyl]amino]-2-fluorobenzoyl]piperazine-1-carbonyl]piperazine-1-carboxylate

To a solution of 5-amino-1-[4-(cyclopropyloxy)-2-(difluoromethyl)-3-fluorophenyl]imidazole-4-carbonyl chloride (74 mg, 0.21 mmol, 1.0 eq) in CH₂Cl₂ (3 mL) were added tert-butyl 4-[4-(4-amino-2-fluorobenzoyl)piperazine-1-carbonyl]piperazine-1-carboxylate (65 mg, 0.15 mmol, 0.7 eq) and pyridine (84 mg, 1.07 mmol, 5.0 eq) in order under N₂ atmosphere. After stirring for 1 h at 25° C., the reaction mixture was concentrated and diluted with EtOAc (30 mL), washed with citric acid aqueous solution (2×15 mL) and H₂O (15 mL), dried over Na₂SO₄ and concentrated under reduced pressure to give the title compound (144 mg, 0.19 mmol, 90% yield). MS: 745.2 [M+H]+, ESI pos

### Step 2: 5-Amino-1-[4-(cyclopropyloxy)-2-(difluoromethyl)-3-fluorophenyl]-N-[3-fluoro-4-[4-(piperazine-1-carbonyl)piperazine-1-carbonyl]phenyl]imidazole-4-carboxamide;2,2,2-trifluoroacetic acid

To a solution of tert-butyl 4-[4-[4-[[5-amino-1-[4-(cyclopropyloxy)-2-(difluoromethyl)-3-fluorophenyl]imidazole-4-carbonyl]amino]-2-fluorobenzoyl]piperazine-1-carbonyl]piperazine-1-carboxylate (144 mg, 0.19 mmol, 1.0 eq) in CH₂Cl₂ (3 mL) was added trifluoroacetic acid (3.0 mL, 39 mmol, 200 eq). The resultant mixture was stirred at 25° C. for 1 h and then concentrated under reduced pressure. The residue was purified by prep-HPLC [Gemini-C18 150×21.2 mm, 5 μm, MeCN— H₂O (0.1% TFA), 40-55% B] to the title compound (85.2 mg, 0.11 mmol, 57% yield). MS: 645.2 [M+H]+, ESI pos

### Compound 382

### 4-[4-[[5-Amino-1-[2-(difluoromethyl)-3-fluoro-4-(fluoromethoxy)phenyl]imidazole-4-carbonyl]amino]-2-fluorobenzoyl]-N-(azetidin-3-yl)piperazine-1-carboxamide;2,2,2-trifluoroacetic acid

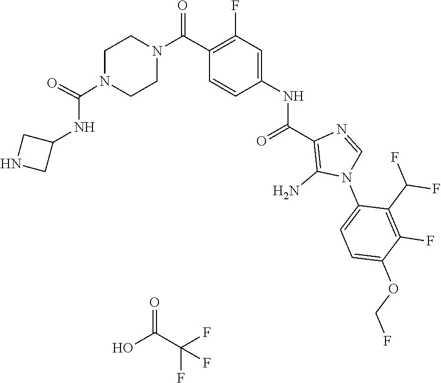 [SMILES: Nc1c(C(=O)Nc2ccc(C(=O)N3CCN(C(=O)NC4CNC4)CC3)c(F)c2)ncn1-c1ccc(OCF)c(F)c1C(F)F ; O=C(O)C(F)(F)F]

### Step 1: tert-Butyl 3-[[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]amino]azetidine-1-carboxylate

A solution of 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (Intermediate 67, 80 mg, 0.15 mmol, 1.0 eq) and triphosgene (27 mg, 0.09 mmol, 0.6 eq) in CH₂Cl₂ (5 mL) was stirred at 25° C. for 0.3 h. And then added 1-BOC-3-(amino) azetidine (52 mg, 0.31 mmol, 2.0 eq) and NEt₃ (0.21 mL, 1.53 mmol, 10.0 eq) was stirred at 25° C. for 1 h. The reaction was concentrated to dryness. The crude material was then purified by flash column chromatography to afford the title compound (47.0 mg, 0.07 mmol, 43% yield). MS: 723.2 [M+H]+

### Step 2: 4-[4-[[5-Amino-1-[2-(difluoromethyl)-3-fluoro-4-(fluoromethoxy)phenyl]imidazole-4-carbonyl]amino]-2-fluorobenzoyl]-N-(azetidin-3-yl)piperazine-1-carboxamide;2,2,2-trifluoroacetic acid

A solution of tert-butyl 3-[[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]amino]azetidine-1-carboxylate (37 mg, 0.05 mmol, 1.0 eq) and trifluoroacetic acid (5 mg, 0.05 mmol, 1.0 eq) in CH₂Cl₂ (1 mL) was stirred at 25° C. for 1 h. The resulting solution was concentrated under reduced pressure. The crude product was purified by Prep-HPLC (Gemini-C18 150×21.2 mm, 5 μm, MeCN— H₂O (0.1% TFA), 15-45% B) to give the title compound (9.9 mg, 0.01 mmol, 30% yield). MS: 623.1 [M+H]+

### Compound 383

### 5-Amino-N-[4-[4-(3-aminoazetidine-1-carbonyl)piperazine-1-carbonyl]-3-fluorophenyl]-1-[2-(difluoromethyl)-3-fluoro-4-(fluoromethoxy)phenyl]imidazole-4-carboxamide;2,2,2-trifluoroacetic acid

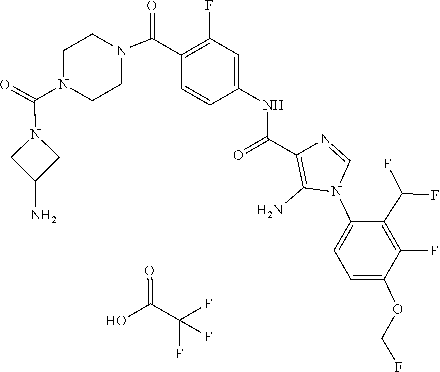 [SMILES: Nc1c(C(=O)Nc2ccc(C(=O)N3CCN(C(=O)N4CC(N)C4)CC3)c(F)c2)ncn1-c1ccc(OCF)c(F)c1C(F)F ; O=C(O)C(F)(F)F]

### Step 1: tert-Butyl N-[1-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]azetidin-3-yl]carbamate

A solution of 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (Intermediate 67, 80 mg, 0.15 mmol, 1.0 eq) and triphosgene (27 mg, 0.09 mmol, 0.6 eq) in CH₂Cl₂ (5 mL) was stirred at 25° C. for 0.3 h, then added 3-N-BOC-amino-azetidine (52 mg, 0.31 mmol, 2.0 eq) and NEt₃ (154 mg, 1.53 mmol, 10.0 eq) was stirred at 25° C. for 1 h. The reaction was concentrated to dryness to afford crude product. The crude was then purified by flash column chromatography eluting to give the title compound (85 mg, 0.12 mmol, 59% yield). MS: 723 [M+H]+ ESI pos

### Step 2: 5-Amino-N-[4-[4-(3-aminoazetidine-1-carbonyl)piperazine-1-carbonyl]-3-fluorophenyl]-1-[2-(difluoromethyl)-3-fluoro-4-(fluoromethoxy)phenyl]imidazole-4-carboxamide;2,2,2-trifluoroacetic acid

A solution of tert-butyl N-[1-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]azetidin-3-yl]carbamate (85 mg, 0.12 mmol, 1.0 eq), trifluoroacetic acid (1.0 mL, 13 mmol, 110 eq) in CH₂Cl₂ (4 mL) was stirred at 25° C. for 1 h. The resulting solution was concentrated under reduced pressure. The crude product was purified by Prep-HPLC (Gemini-C18 150×21.2 mm, 5 μm, MeCN— H₂O (0.1% TFA), 30-60% B) to afford the title compound (14 mg, 0.02 mmol, 15.97% yield). MS: 623.2 [M+H]⁺ ESI pos

### Compound 384

### 4-[4-[[5-Amino-1-[2-(difluoromethyl)-3-fluoro-4-(fluoromethoxy)phenyl]imidazole-4-carbonyl]amino]-2-fluorobenzoyl]-N-(azetidin-3-ylmethyl)piperazine-1-carboxamide;2,2,2-trifluoroacetic acid

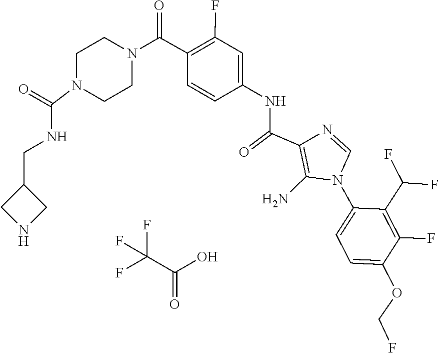 [SMILES: Nc1c(C(=O)Nc2ccc(C(=O)N3CCN(C(=O)NCC4CNC4)CC3)c(F)c2)ncn1-c1ccc(OCF)c(F)c1C(F)F ; O=C(O)C(F)(F)F]

### Step 1: tert-Butyl 3-[[[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]amino]methyl]azetidine-1-carboxylate

A solution of 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (Intermediate 67, 80 mg, 0.15 mmol, 1.0 eq) and triphosgene (27 mg, 0.09 mmol, 0.6 eq) in CH₂Cl₂ (5 mL) was stirred at 25° C. for 0.3 h, then NEt₃ (77 mg, 0.76 mmol, 5.0 eq) and 1-BOC-3-(aminomethyl) azetidine (56 mg, 0.31 mmol, 2.0 eq) were added in order, the resultant mixture was stirred at 25° C. for 1 h. The reaction was concentrated to dryness. The crude material was then purified by flash column chromatography on silica gel (CH₂Cl₂:MeOH=93:7) to afford the title compound (73 mg, 0.1 mmol, 44% yield). MS: 737.2 [M+H]⁺ ESI pos

### Step 2: 4-[4-[[5-Amino-1-[2-(difluoromethyl)-3-fluoro-4-(fluoromethoxy)phenyl]imidazole-4-carbonyl]amino]-2-fluorobenzoyl]-N-(azetidin-3-ylmethyl)piperazine-1-carboxamide;2,2,2-trifluoroacetic acid

To a solution of tert-butyl 3-[[[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]amino]methyl]azetidine-1-carboxylate (73 mg, 0.07 mmol, 1.0 eq) in CH₂Cl₂ (2 mL) was added trifluoroacetic acid (0.5 mL, 6.53 mmol, 98.35 eq). The resulting mixture was stirred at 25° C. for 1 h before cconcentrating under reduced pressure. The residue was purified by prep-HPLC [Gemini-C18 150×21.2 mm, 5 μm, MeCN— H₂O (0.1% TFA), 20-50% B] to afford the title compound (7.4 mg, 0.01 mmol, 15% yield). MS: 637.2 [M+H]+ ESI pos

### Compound 385

### 5-Amino-N-[3-chloro-4-[4-(piperazine-1-carbonyl)piperidine-1-carbonyl]phenyl]-1-[2-(difluoromethyl)-3-fluoro-4-(fluoromethoxy)phenyl]imidazole-4-carboxamide;2,2,2-trifluoroacetic acid

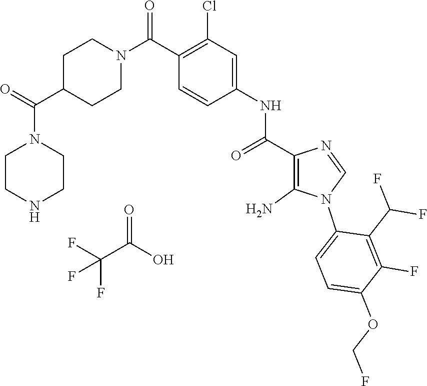 [SMILES: Nc1c(C(=O)Nc2ccc(C(=O)N3CCC(C(=O)N4CCNCC4)CC3)c(Cl)c2)ncn1-c1ccc(OCF)c(F)c1C(F)F ; O=C(O)C(F)(F)F]

### Step 1: tert-Butyl 4-[1-[4-[[5-amino-1-[2-(difluoromethyl)-3-fluoro-4-(fluoromethoxy)phenyl]imidazole-4-carbonyl]amino]-2-chlorobenzoyl]piperidine-4-carbonyl]piperazine-1-carboxylate

To a solution of tert-butyl 4-[1-(4-amino-2-chlorobenzoyl)piperidine-4-carbonyl]piperazine-1-carboxylate (74 mg, 0.17 mmol, 0.7 eq) in CH₂Cl₂ (3 mL) was added pyridine (374 mg, 4.74 mmol, 20.0 eq) and 5-amino-1-[2-(difluoromethyl)-3-fluoro-4-(fluoromethoxy)phenyl]imidazole-4-carbonyl chloride (80 mg, 0.24 mmol, 1.0 eq). The reaction mixture was stirred at 25° C. for 16 h before concentration to dryness. The residue was taken up in CH₂Cl₂ (20 mL) and the organics washed with H₂O (2×20 mL) then brine (20 mL). The organics were then separated and dried (MgSO₄) before concentrating to dryness. The crude was then purified by flash column chromatography eluting 12% MeOH in CH₂Cl₂. The desired fractions were concentrated to dryness under reduced pressure. to afford the title compound (80 mg, 0.11 mmol, 45% yield). MS 752.2 [M+H]+, ESI pos

### Step 2: 5-Amino-N-[3-chloro-4-[4-(piperazine-1-carbonyl)piperidine-1-carbonyl]phenyl]-1-[2-(difluoromethyl)-3-fluoro-4-(fluoromethoxy)phenyl]imidazole-4-carboxamide;2,2,2-trifluoroacetic acid

To a solution of tert-butyl 4-[1-[4-[[5-amino-1-[2-(difluoromethyl)-3-fluoro-4-(fluoromethoxy)phenyl]imidazole-4-carbonyl]amino]-2-chlorobenzoyl]piperidine-4-carbonyl]piperazine-1-carboxylate (70 mg, 0.09 mmol, 1.0 eq) in CH₂Cl₂ (3 mL) was added trifluoroacetic acid (1.0 mL). The reaction stirred at 25° C. for 1 h and was then concentrated to dryness. The crude was then purified by HPLC (Gemini-C18 150×21.2 mm, 5 μm, MeCN— H₂O (0.1% TFA), 20-60% B) to afford the title compound (29.7 mg, 0.04 mmol, 40% yield). MS: 652.0 [M+H]+ ESI pos

### Compound 386

### 5-Amino-N-[4-[4-[2-(azetidin-3-yl) acetyl]piperazine-1-carbonyl]-3-chlorophenyl]-1-[2-(difluoromethyl)-3-fluoro-4-(fluoromethoxy)phenyl]imidazole-4-carboxamide;2,2,2-trifluoroacetic acid

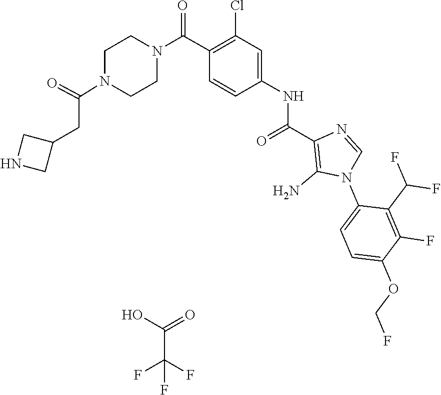 [SMILES: Nc1c(C(=O)Nc2ccc(C(=O)N3CCN(C(=O)CC4CNC4)CC3)c(Cl)c2)ncn1-c1ccc(OCF)c(F)c1C(F)F ; O=C(O)C(F)(F)F]

### Step 1: tert-Butyl 3-[2-[4-[4-[[5-amino-1-[2-(difluoromethyl)-3-fluoro-4-(fluoromethoxy)phenyl]imidazole-4-carbonyl]amino]-2-chlorobenzoyl]piperazin-1-yl]-2-oxo-ethyl]azetidine-1-carboxylate

To a solution of 5-amino-N-[3-chloro-4-(piperazine-1-carbonyl)phenyl]-1-[2-(difluoromethyl)-3-fluoro-4-(fluoromethoxy)phenyl]imidazole-4-carboxamide (Intermediate 133, 80.0 mg, 0.15 mmol, 1.0 eq), 2-(1-(tert-butoxycarbonyl) azetidin-3-yl) acetic acid (22 mg, 0.1 mmol, 0.7 eq), propylphosphonic anhydride (470 mg, 0.74 mmol, 5.0 eq) and diisopropylethylamine (0.15 mL, 0.89 mmol, 6.0 eq). The resultant mixture was stirred at 25° C. for 3 h. The reaction was concentrated to dryness and the residue was taken up in EtOAc (5 ml) and the organics washed with H₂O (2×3 ml) then saturated bring (3 ml). The organics were then separated and dried (Na₂SO₄) before concentration to dryness. The crude was then purified by flash column chromatography eluting 60% EtOAc in PE. The desired fractions were concentrated to dryness under reduced pressure to afford the title compound (70 mg, 0.09 mmol, 64% yield). MS: 738.2 [M+H]+

### Step 2: 5-Amino-N-[4-[4-[2-(azetidin-3-yl) acetyl]piperazine-1-carbonyl]-3-chlorophenyl]-1-[2-(difluoromethyl)-3-fluoro-4-(fluoromethoxy)phenyl]imidazole-4-carboxamide;2,2,2-trifluoroacetic acid

To a solution of tert-butyl 3-[2-[4-[4-[[5-amino-1-[2-(difluoromethyl)-3-fluoro-4-(fluoromethoxy)phenyl]imidazole-4-carbonyl]amino]-2-chlorobenzoyl]piperazin-1-yl]-2-oxo-ethyl]azetidine-1-carboxylate (40 mg, 0.05 mmol, 1.0 eq) and trifluoroacetic acid (1.0 mL, 13 mmol, 240 eq) in CH₂Cl₂ (3 mL) was stirred at 25° C. for 1 h. The resulting solution was concentrated under reduced pressure. The residue was purified by Prep-HPLC (Gemini-C18 150×21.2 mm, 5 μm, MeCN— H₂O (0.1% TFA), 25-40% B) to afford the title compound (6.2 mg, 0.01 mmol, 14% yield). MS: 638.2 [M+H]+ ESI pos

### Compound 387

### 5-Amino-1-[2-(difluoromethyl)-3-fluoro-4-(fluoromethoxy)phenyl]-N-[3-fluoro-4-[4-[(2R,4R)-4-methylazetidine-2-carbonyl]piperazine-1-carbonyl]phenyl]imidazole-4-carboxamide;2,2,2-trifluoroacetic acid

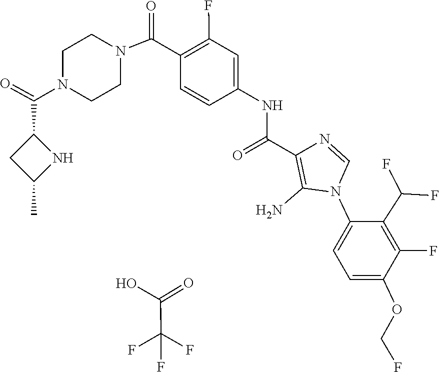 [SMILES: C[C@@H]1C[C@H](C(=O)N2CCN(C(=O)c3ccc(NC(=O)c4ncn(-c5ccc(OCF)c(F)c5C(F)F)c4N)cc3F)CC2)N1 ; O=C(O)C(F)(F)F]

### Step 1:(2R,4R)-1-tert-Butoxycarbonyl-4-methylazetidine-2-carboxylic acid

A solution of (2R,4R)-4-methylazetidine-2-carboxylic acid (50 mg, 0.43 mmol, 1.0 eq), di-tert-butyl dicarbonate (285 mg, 1.3 mmol, 3.0 eq), and NEt₃ (0.3 mL, 2.17 mmol, 5.0 eq) in THF (2 mL) was stirred at 25° C. for 1 h. The resulting solution was concentrated under reduced pressure. The residue was applied on a silica gel column and eluted with i-PrOH/PE (1/1) to afford the title compound (70 mg, 0.33 mmol, 75% yield). MS: 214.1 [M−H]−, ESI neg

### Step 2: tert-Butyl(2R,4R)-2-[4-[4-[[5-Amino-1-[2-(difluoromethyl)-3-fluoro-4-(fluoromethoxy)phenyl]imidazole-4-carbonyl]amino]-2-fluorobenzoyl]piperazine-1-carbonyl]-4-methylazetidine-1-carboxylate

A solution of 5-amino-1-[2-(difluoromethyl)-3-fluoro-4-(fluoromethoxy)phenyl]-N-[3-fluoro-4-(piperazine-1-carbonyl)phenyl]imidazole-4-carboxamide (Intermediate 67, 70 mg, 0.13 mmol, 1.0 eq), (2R,4R)-1-tert-butoxycarbonyl-4-methylazetidine-2-carboxylic acid (28 mg, 0.13 mmol, 1.0 eq), propylphosphonic anhydride (424 mg, 0.67 mmol, 5.0 eq) and diisopropylethylamine (0.14 mL, 0.8 mmol, 6.0 eq) was stirred at 25° C. for 3 h. The reaction was concentrated to dryness and the residue was taken up in EtOAc (5 ml) and the organics washed with H₂O (2×3 ml) then brine (3 ml). The organics were then separated and dried (Na₂SO₄) before concentration to dryness. The crude was then purified by flash column chromatography eluting 60% EtOAc in PE. The desired fractions were concentrated to dryness under reduced pressure to the title compound (75 mg, 0.1 mmol, 78% yield). MS: 622.0 [M+H−Boc], ESI pos

### Step 3: 5-Amino-1-[2-(difluoromethyl)-3-fluoro-4-(fluoromethoxy)phenyl]-N-[3-fluoro-4-[4-[(2R,4R)-4-methylazetidine-2-carbonyl]piperazine-1-carbonyl]phenyl]imidazole-4-carboxamide;2,2,2-trifluoroacetic acid

To a solution of tert-butyl(2R,4R)-2-[4-[4-[[5-amino-1-[2-(difluoromethyl)-3-fluoro-4-(fluoromethoxy)phenyl]imidazole-4-carbonyl]amino]-2-fluorobenzoyl]piperazine-1-carbonyl]-4-methylazetidine-1-carboxylate (50 mg, 0.07 mmol, 1.0 eq) and trifluoroacetic acid (1.0 mL, 13 mmol, 190 eq) in CH₂Cl₂ (3 mL) was stirred at 25° C. for 1 h. The resulting solution was concentrated under reduced pressure. The residue was purified by Prep-HPLC (-Gemini-C18 150×21.2 mm, 5 μm, MeCN— H₂O (0.1% TFA), 25-40) to afford the title compound (11.1 mg, 0.02 mmol, 22% yield). MS: 622.2 [M+H]+ ESI pos

### Compound 388

### 5-Amino-1-[1-(5-amino-3-fluoro-2-pyridyl)-3-(trifluoromethyl)pyrazol-4-yl]-N-[3-chloro-4-[4-[(1S,5R,6r)-3-azabicyclo[3.1.0]hexane-6-carbonyl]piperazine-1-carbonyl]phenyl]imidazole-4-carboxamide;2,2,2-trifluoroacetic acid

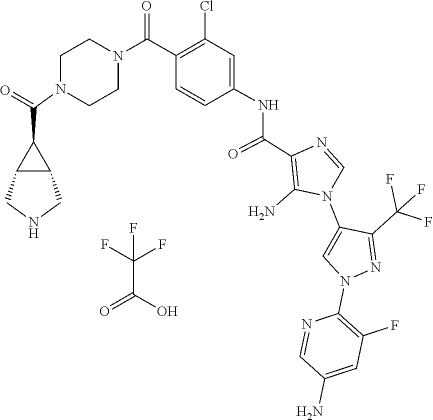 [SMILES: Nc1cnc(-n2cc(-n3cnc(C(=O)Nc4ccc(C(=O)N5CCN(C(=O)[C@H]6[C@@H]7CNC[C@@H]76)CC5)c(Cl)c4)c3N)c(C(F)(F)F)n2)c(F)c1 ; O=C(O)C(F)(F)F]

### Step 1: tert-Butyl (1R,5S,6r)-6-[4-[4-[[5-Amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate

To a solution of 5-amino-N-[3-chloro-4-(piperazine-1-carbonyl)phenyl]-1-[3-(trifluoromethyl)-1H-pyrazol-4-yl]imidazole-4-carboxamide (Intermediate 77, free base, 2.55 g, 5.28 mmol, 1.0 eq) in CH₂Cl₂ (20 mL) were added (1S,5R,6r)-3-tert-butoxycarbonyl-3-azabicyclo[3.1.0]hexane-6-carboxylic acid (0.84 g, 3.7 mmol, 0.7 eq), propyl phosphate anhydride (5.04 g, 7.92 mmol, 1.5 eq) and N,N-diisopropylethylamine (3.68 mL, 21.12 mmol, 4.0 eq). The resultant mixture was stirred at 0° C. for 1 h before the solvent was removed under reduced pressure. The residue was diluted with EtOAc (10 mL), washed with H₂O (4×10 mL), dried over Na₂SO₄ and concentrated under reduced pressure to afford the title compound (3.1 g, 4.48 mmol, 85% yield). MS [M-tBu+H]+: 636.1

### Step 2: tert-Butyl (1S,5R,6r)-6-[4-[4-[[5-amino-1-[1-(3-fluoro-5-nitro-2-pyridyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of tert-butyl(1S,5R,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (100 mg, 0.14 mmol, 1.0 eq) in the DMF (1 mL) was added 2-chloro-3-fluoro-5-nitro-pyridine (30 mg, 0.17 mmol, 1.2 eq) and K₂CO₃ (59 mg, 0.43 mmol, 3.0 eq) the reaction was stirred at 60° C. for 3 h. The reaction was concentrated to dryness and the residue was taken up in EtOAc (20 ml) and the organics washed with H₂O (2×10 ml) then brine (10 ml). The organics were then separated and dried (Na₂SO₄) before concentration to dryness to afford the title compound (65 mg, 0.08 mmol, 40% yield). MS: 732.0 [M+H−Boc]+

### Step 3: tert-Butyl (1S,5R,6r)-6-[4-[4-[[5-Amino-1-[1-(5-amino-3-fluoro-2-pyridyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A mixture of tert-butyl(1S,5R,6r)-6-[4-[4-[[5-amino-1-[1-(3-fluoro-5-nitro-2-pyridyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (50 mg, 0.06 mmol, 1.0 eq), Fe powder (16.78 mg, 0.3 mmol, 5.0 eq) and NH₄Cl (16 mg, 0.3 mmol, 5.0 eq) in EtOH (3 mL) and H₂O (0.6 mL) was stirred at 50° C. for 2 h. The solid was filtered. The crude was then purified by flash column chromatography eluting 40% EtOAc in PE. The desired fractions were concentrated to dryness under reduced pressure to afford the title compound (35 mg, 0.04 mmol, 72% yield) as a yellow oil. MS: 801.9 [M+H]+

### Step 4: 5-Amino-1-[1-(5-amino-3-fluoro-2-pyridyl)-3-(trifluoromethyl)pyrazol-4-yl]-N-[3-chloro-4-[4-[(1S,5R,6r)-3-azabicyclo[3.1.0]hexane-6-carbonyl]piperazine-1-carbonyl]phenyl]imidazole-4-carboxamide;2,2,2-trifluoroacetic acid

To a solution of tert-butyl(1S,5R,6r)-6-[4-[4-[[5-amino-1-[1-(5-amino-3-fluoro-2-pyridyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (30 mg, 0.04 mmol, 1.0 eq) and trifluoroacetic acid (1.0 mL, 13 mmol, 350 eq) in CH₂Cl₂ (3 mL) was stirred at 25° C. for 1 h. The resulting solution was concentrated under reduced pressure. The residue was purified by Prep-HPLC (Gemini-C18 150×21.2 mm, 5 μm, MeCN— H₂O (0.1% TFA), 25-40% B) to afford the title compound (6.6 mg, 0.01 mmol, 21% yield). MS: 702.0 [M+H]+, ESI pos

### Compound 389

### 5-Amino-N-[3-chloro-4-[4-[(1S,5R,6r)-3-azoniabicyclo[3.1.0]hexane-6-carbonyl]piperazine-1-carbonyl]phenyl]-1-[1-(2,2,2-trifluoroethyl)-3-(trifluoromethyl)pyrazol-4-yl]imidazole-4-carboxamide; formate

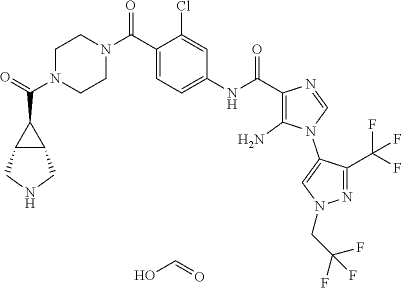 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC(F)(F)F)nc1C(F)(F)F ; O=CO]

### Step 1: tert-Butyl (1S,5R,6r)-6-[4-[4-[[5-amino-1-[1-(2,2,2-trifluoroethyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of 2,2,2-trifluoroethyl trifluoromethanesulfonate (100 mg, 0.43 mmol, 1.5 eq), tert-butyl(1S,5R,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (200 mg, 0.29 mmol, 1.0 eq) and K₂CO₃ (119 mg, 0.87 mmol, 3.0 eq) in DMF (5 mL) was stirred at 50° C. for 1 h. The reaction was concentrated under reduced pressure. The residue was applied on a silica gel column and eluted with MeOH/CH₂Cl₂ (1/20) to afford the title compound (180 mg, 0.23 mmol, 80% yield). MS: 717.7 [(M+H−tBu] ESI pos.

### Step 2: 5-Amino-N-[3-chloro-4-[4-[(1S,5R,6r)-3-azoniabicyclo[3.1.0]hexane-6-carbonyl]piperazine-1-carbonyl]phenyl]-1-[1-(2,2,2-trifluoroethyl)-3-(trifluoromethyl)pyrazol-4-yl]imidazole-4-carboxamide; formate

A solution of tert-butyl(1S,5R,6r)-6-[4-[4-[[5-amino-1-[1-(2,2,2-trifluoroethyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (40 mg, 0.05 mmol, 1.0 eq) and hydrochloric acid in dioxane (4.0 mL, 16 mmol, 310 eq) in MeOH (1 mL) was stirred at 20° C. for 1 h. The resulting solution was concentrated under reduced pressure. The residue was purified by Prep-HPLC (Gemini-C18 150×21.2 mm, 5 μm, MeCN— H₂O (0.1% FA), 35-40% B) to give the title compound (24.4 mg, 0.03 mmol, 65% yield). MS: 674 [M+H]+ ESI pos

### Compound 390

### 5-Amino-N-[3-chloro-4-[4-[(1S,5R,6r)-3-azabicyclo[3.1.0]hexane-6-carbonyl]piperazine-1-carbonyl]phenyl]-1-[1-[(2,2-difluorocyclopropyl)methyl]-3-(trifluoromethyl)pyrazol-4-yl]imidazole-4-carboxamide;2,2,2-trifluoroacetic acid

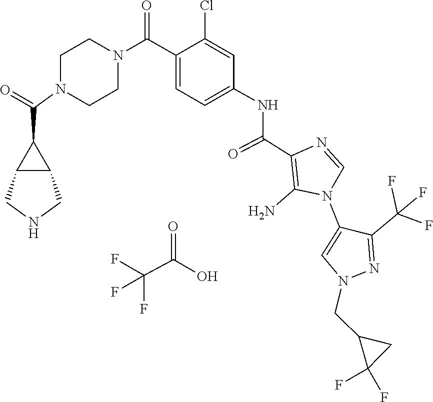 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2CC2(F)F)nc1C(F)(F)F ; O=C(O)C(F)(F)F]

### Step 1: tert-Butyl (1S,5R,6r)-6-[4-[4-[[5-amino-1-[1-[(2,2-difluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of tert-butyl(1S,5R,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (100 mg, 0.14 mmol, 1.0 eq) in the DMF (3 mL) was added 1-bromomethyl-2,2-difluorocyclopropane (49 mg, 0.29 mmol, 2.0 eq) and K₂CO₃ (59 mg, 0.43 mmol, 3.0 eq) the reaction was stirred at 25° C. for 16 h The reaction was concentrated to dryness and the residue was taken up in EtOAc (20 ml) and the organics washed with H₂O (2×10 ml) then brine (10 ml). The organics were then separated and dried (MgSO₄) before concentration to dryness to afford the title compound (91 mg, 0.12 mmol, 80% yield). MS: 725.9 [M+H−tBu]+ ESI pos

### Step 2: 5-Amino-N-[3-chloro-4-[4-[(1S,5R,6r)-3-azabicyclo[3.1.0]hexane-6-carbonyl]piperazine-1-carbonyl]phenyl]-1-[1-[(2,2-difluorocyclopropyl)methyl]-3-(trifluoromethyl)pyrazol-4-yl]imidazole-4-carboxamide;2,2,2-trifluoroacetic acid

A solution of tert-butyl(1S,5R,6r)-6-[4-[4-[[5-amino-1-[1-[(2,2-difluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (47 mg, 0.06 mmol, 1.0 eq) in the CH₂Cl₂ (3 mL) was added trifluoroacetic acid (1.0 mL) the reaction was stirred at 25° C. for 3 h The reaction was concentrated to dryness. The crude material was then purified by by Prep-HPLC (Gemini-C18 150×21.2 mm, 5 μm, MeCN— H₂O (0.1% TFA), 35-40% B) to give the title compound (38 mg, 0.05 mmol, 89% yield). MS: 682 [M+H]+, ESI pos

### Compound 391

### 5-Amino-N-[3-chloro-4-[4-(piperidine-4-carbonyl)piperazine-1-carbonyl]phenyl]-1-[1-(cyclopropylmethyl)-3-(trifluoromethyl)pyrazol-4-yl]imidazole-4-carboxamide;2,2,2-trifluoroacetic acid

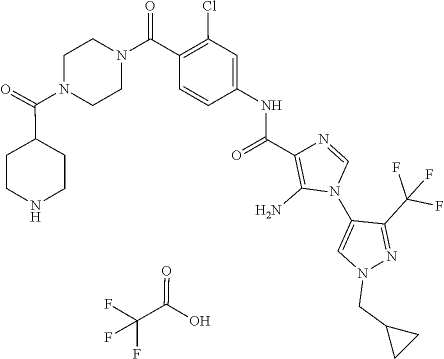 [SMILES: Nc1c(C(=O)Nc2ccc(C(=O)N3CCN(C(=O)C4CCNCC4)CC3)c(Cl)c2)ncn1-c1cn(CC2CC2)nc1C(F)(F)F ; O=C(O)C(F)(F)F]

### Step 1: tert-Butyl 4-[4-[4-[[5-amino-1-[1-(cyclopropylmethyl)-3-(trifluoromethyl)pyrazol-4-yl]imidazole-4-carbonyl]amino]-2-chlorobenzoyl]piperazine-1-carbonyl]piperidine-1-carboxylate

To a solution of tert-butyl 4-[4-[4-[[5-amino-1-[3-(trifluoromethyl)-1H-pyrazol-4-yl]imidazole-4-carbonyl]amino]-2-chlorobenzoyl]piperazine-1-carbonyl]piperidine-1-carboxylate (Intermediate 81, 50 mg, 0.07 mmol, 1.0 eq) in DMF (3 mL) was added (bromomethyl)cyclopropane (0.01 mL, 0.07 mmol, 1.0 eq) and K₂CO₃ (29 mg, 0.22 mmol, 3.0 eq). The reaction was stirred at 25° C. for 16 h before concentrating to dryness. The residue was taken up in EtOAc (20 ml) and the organics washed with H₂O (2×20 ml) then brine (20 ml) to afford the title compound, which was used without further purification. MS: 748 [M+H]+

### Step 2: 5-Amino-N-[3-chloro-4-[4-(piperidine-4-carbonyl)piperazine-1-carbonyl]phenyl]-1-[1-(cyclopropylmethyl)-3-(trifluoromethyl)pyrazol-4-yl]imidazole-4-carboxamide;2,2,2-trifluoroacetic acid

To a solution of tert-butyl 4-[4-[4-[[5-amino-1-[1-(cyclopropylmethyl)-3-(trifluoromethyl)pyrazol-4-yl]imidazole-4-carbonyl]amino]-2-chlorobenzoyl]piperazine-1-carbonyl]piperidine-1-carboxylate (35 mg, 0.05 mmol, 1.0 eq) in DCM (3 mL) was added trifluoroacetic acid (1.0 mL, 13 mmol mmol, 2.6 eq). The reaction was stirred at 25° C. for 3 h. The reaction was concentrated to dryness. The crude was then purified by Prep-HPLC (Gemini-C18 150×21.2 mm, 5 μm, MeCN— H₂O (0.1% TFA), 30-40% B) to afford the title compound (5.9 mg, 0.01 mmol, 16% yield). MS: 648 [M+H]+, ESI pos

### Compound 392

### 5-Amino-N-[4-[4-[(1S,5R,6r)-3-azabicyclo[3.1.0]hexane-6-carbonyl]piperazine-1-carbonyl]-3-chlorophenyl]-1-[1-[(1-fluorocyclopropyl)methyl]-3-(trifluoromethyl)pyrazol-4-yl]imidazole-4-carboxamide

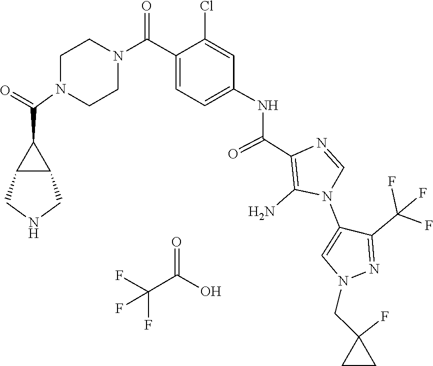 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2(F)CC2)nc1C(F)(F)F ; O=C(O)C(F)(F)F]

### Step 1:(1-Fluorocyclopropyl)methyl 4-methylbenzenesulfonate

To a solution of (1-fluorocyclopropyl)methanol (200 mg, 2.22 mmol, 1.0 eq) in CH₂Cl₂ (3 mL) was added p-toluenesulphonyl chloride (846 mg, 4.44 mmol, 2.0 eq). The reaction was stirred at 0° C. for 0.5 h before addition of p-toluenesulphonyl chloride (846 mg, 4.44 mmol, 2.0 eq). The reaction was stirred at 50° C. for 4 h. The resulting solution was diluted with 10 ml of H₂O, then extracted with EtOAc (20 ml) and the organics washed with H₂O (2×10 ml) then saturated brine (10 ml). The organics were then separated and dried with MgSO₄ before concentration to dryness. The crude was then purified by flash column chromatography eluting 10% EtOAc in isohexane. The desired fractions were concentrated to dryness under reduced pressure. to afford the title compound (350 mg, 1.43 mmol, 64% yield) as colorless oil. MS [M+Na]+: 267.0

### Step 2: tert-Butyl (1S,5R,6r)-6-[4-[4-[[5-amino-1-[1-[(1-fluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl(1S,5R,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (550 mg, 0.79 mmol, 1.0 eq) in DMF (8 mL) was added K₂CO₃ (366 mg, 2.65 mmol, 3.33 eq) and (1-fluorocyclopropyl)methyl 4-methylbenzenesulfonate (485 mg, 1.99 mmol, 2.5 eq). The reaction was stirred at 50° C. for 16 h before concentration to dryness. The residue was taken up in EtOAc (20 ml) and the organics washed with H₂O (2×20 ml) then brine (20 ml). The organics were then separated and dried (MgSO₄) before concentration to dryness. The crude was then purified by flash column chromatography eluting with 10% MeOH in CH₂Cl₂. The desired fractions were concentrated to dryness under reduced pressure to afford the title compound (513 mg, 0.67 mmol, 84% yield). MS: [M-tBu+H]+=708.1

### Step 3: 5-Amino-N-[4-[4-[(1S,5R,6r)-3-azabicyclo[3.1.0]hexane-6-carbonyl]piperazine-1-carbonyl]-3-chlorophenyl]-1-[1-[(1-fluorocyclopropyl)methyl]-3-(trifluoromethyl)pyrazol-4-yl]imidazole-4-carboxamide

To a solution of tert-butyl(1R,5S,6r)-6-[4-[4-[[5-amino-1-[1-[(1-fluorocyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (100 mg, 0.13 mmol, 1.0 eq) in CH₂Cl₂ (3 mL) was added trifluoroacetic acid (1.0 mL). The reaction was stirred at 25° C. for 3 h before concentration the title compound (68 mg, 0.1 mmol, 73% yield). MS: 664.2 [M+H]+

### Compound 393

### 5-Amino-N-[3-chloro-4-[4-[(1S,5R,6r)-3-azabicyclo[3.1.0]hexane-6-carbonyl]piperazine-1-carbonyl]phenyl]-1-[3-(trifluoromethyl)-1-[[1-(trifluoromethyl)cyclopropyl]methyl]pyrazol-4-yl]imidazole-4-carboxamide;2,2,2-trifluoroacetic acid

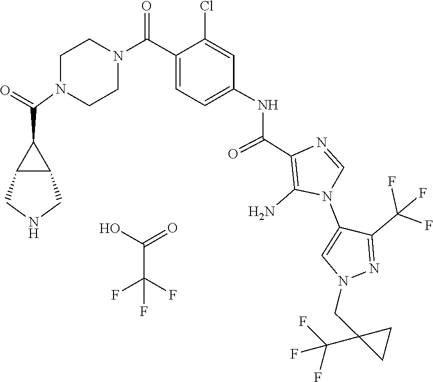 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2(C(F)(F)F)CC2)nc1C(F)(F)F ; O=C(O)C(F)(F)F]

### Step 1: [1-(trifluoromethyl)cyclopropyl]methyl 4-methylbenzenesulfonate

A solution of [1-(trifluoromethyl)cyclopropyl]methanol (100 mg, 0.71 mmol, 1.0 eq), 4-dimethylaminopyridine (17 mg, 0.14 mmol, 0.2 eq), tosyl chloride (163.3 mg, 0.86 mmol, 1.2 eq) and triethylamine (0.2 mL, 1.43 mmol, 2.0 eq) in DCM (2 mL) was stirred at 25° C. for 16 h. The reaction was concentrated to dryness. The crude was then purified by flash column chromatography eluting 15% EtOAc in PE. The desired fractions were concentrated to dryness in vacuo to afford the title compound (150 mg, 0.51 mmol, 71.41% yield) as a colorless liquid. MS: 317.0 [M+Na]+

### Step 2: tert-Butyl (1S,5R,6r)-6-[4-[4-[[5-amino-1-[3-(trifluoromethyl)-1-[[1-(trifluoromethyl)cyclopropyl]methyl]pyrazol-4-yl]imidazole-4-carbonyl]amino]-2-chloro-benzoyl]piperazine-1-carbonyl]-3-azabicyclo[3.1.0]hexane-3-carboxylate

To a solution of tert-butyl(1R,5S,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (50 mg, 0.07 mmol, 1.0 eq) in the DMF (2 mL) was added [1-(trifluoromethyl)cyclopropyl]methyl 4-methylbenzenesulfonate (25 mg, 0.09 mmol, 1.2 eq) and K₂CO₃ (29 mg, 0.22 mmol, 3.0 eq). The reaction was stirred at 25° C. for 3 h before solvent removal under reduced pressure. The residue was taken up in EtOAc (20 ml) and the organics washed with H₂O (2×10 ml) then brine (10 ml). The organics were then separated and dried (Na₂SO₄) before concentration to dryness to give the title compund (50 mg, 0.07 mmol, 1.0 eq). MS: 814.0 [M+H]+

### Step 3: 5-Amino-N-[3-chloro-4-[4-[(1S,5R,6r)-3-azabicyclo[3.1.0]hexane-6-carbonyl]piperazine-1-carbonyl]phenyl]-1-[3-(trifluoromethyl)-1-[[1-(trifluoromethyl)cyclopropyl]methyl]pyrazol-4-yl]imidazole-4-carboxamide;2,2,2-trifluoroacetic acid

To a solution of tert-butyl(1S,5R,6r)-6-[4-[4-[[5-amino-1-[3-(trifluoromethyl)-1-[[1-(trifluoromethyl)cyclopropyl]methyl]pyrazol-4-yl]imidazole-4-carbonyl]amino]-2-chloro-benzoyl]piperazine-1-carbonyl]-3-azabicyclo[3.1.0]hexane-3-carboxylate (30 mg, 0.04 mmol, 1.0 eq) and trifluoroacetic acid (1.0 mL, 12.98 mmol, 352.26 eq) in DCM (3 mL) was stirred at 25° C. for 1 h. The resulting solution was concentrated under vacuum. The residue was purified by Prep-HPLC (-Gemini-C18 150×21.2 mm, 5 um, ACN— H₂O (0.1% TFA)) to afford the title compound (19 mg, 0.02 mmol, 62.27% yield). MS: 714.2 [M+H]+

### Compound 394

### 5-Amino-N-[3-chloro-4-[4-[(1S,5R,6r)-3-azabicyclo[3.1.0]hexane-6-carbonyl]piperazine-1-carbonyl]phenyl]-1-[1-[(1-methylcyclopropyl)methyl]-3-(trifluoromethyl)pyrazol-4-yl]imidazole-4-carboxamide

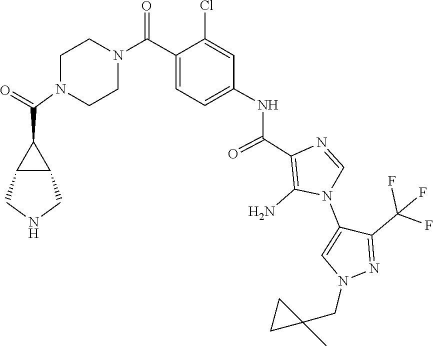 [SMILES: CC1(Cn2cc(-n3cnc(C(=O)Nc4ccc(C(=O)N5CCN(C(=O)[C@H]6[C@@H]7CNC[C@@H]76)CC5)c(Cl)c4)c3N)c(C(F)(F)F)n2)CC1]

### Step 1:(1-Methylcyclopropyl)methyl methanesulfonate

A solution of 1-methylcyclopropanemethanol (20 mg, 0.23 mmol, 1.0 eq), NEt₃ (46 mg, 0.46 mmol, 2.0 eq), and methanesulphonyl chloride (0.03 mL, 0.35 mmol, 1.5 eq) in CH₂Cl₂ (1 mL) was stirred at 0° C. for 1 h. The residue was washed with NaHCO₃(aqueous), dried, and concentrated under reduced pressure to give the title compound (30 mg, 0.18 mmol, 79% yield). 1H NMR (400 MHz,) δ 4.00 (s, 2H), 3.01 (s, 3H), 1.18 (s, 2H), 0.56 (t, J=5.3 Hz, 1H), 0.46 (t, J=5.4 Hz, 1H).

### Step 2: tert-Butyl (1S,5R,6r)-6-[4-[4-[[5-amino-1-[1-[(1-methylcyclopropyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl(1R,5S,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (50 mg, 0.07 mmol, 1.0 eq) in the DMF (2 mL) was added (1-methylcyclopropyl)methyl methanesulfonate (14 mg, 0.09 mmol, 1.2 eq) and K₂CO₃ (29 mg, 0.22 mmol, 3.0 eq). The reaction was stirred at 25° C. for 3 h and then concentrated to dryness. The residue was taken up in EtOAc (20 ml) and the organics washed with H₂O (2×10 ml) then brine (10 ml). The organics were then separated and dried (Na₂SO₄) before concentration to dryness to the title compound (40 mg, 0.05 mmol, 60% yield). MS: 660.2 [M+H−Boc]+

### Step 3: 5-Amino-N-[3-chloro-4-[4-[(1S,5R,6r)-3-azabicyclo[3.1.0]hexane-6-carbonyl]piperazine-1-carbonyl]phenyl]-1-[1-[(1-methylcyclopropyl)methyl]-3-(trifluoromethyl)pyrazol-4-yl]imidazole-4-carboxamide

A solution of tert-butyl(1S,5R,6r)-6-[4-[4-[[5-amino-1-[1-[(1-methylcyclopropyl)methyl]-3-(trifluoromethyl)pyrazol-4-yl]imidazole-4-carbonyl]amino]-2-chloro-benzoyl]piperazine-1-carbonyl]-3-azabicyclo[3.1.0]hexane-3-carboxylate (40 mg, 0.05 mmol, 1.0 eq) and trifluoroacetic acid (1.0 mL, 12.98 mmol, 246.69 eq) in DCM (3 mL) was stirred at 25° C. for 1 h. The resulting solution was concentrated under vacuum. The residue was purified by Prep-HPLC (chiralpak-OJ, CO2-ETOH(DEA)) to afford the title compound (2.9 mg, 0.0 mmol, 8.21% yield). MS: 660.1 [M+H]+

### Compound 395

### 5-Amino-N-[4-[4-[(1S,5R,6r)-3-azabicyclo[3.1.0]hexane-6-carbonyl]piperazine-1-carbonyl]-3-chlorophenyl]-1-[1-(1-bicyclo[1.1.1]pentanylmethyl)-3-(trifluoromethyl)pyrazol-4-yl]imidazole-4-carboxamide;2,2,2-trifluoroacetic acid

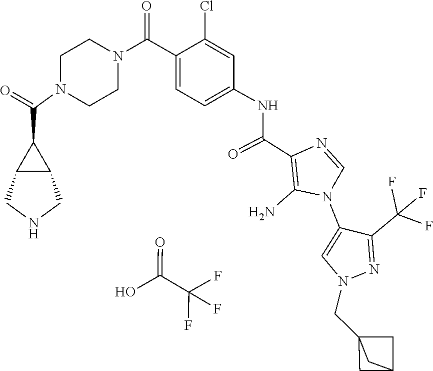 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC23CC(C2)C3)nc1C(F)(F)F ; O=C(O)C(F)(F)F]

### Step 1: 1-Bicyclo[1.1.1]pentanylmethyl 4-methylbenzenesulfonate

A solution of 1-bicyclo[1.1.1]pentylmethanol (91 mg, 0.93 mmol, 1.0 eq), 4-dimethylaminopyridine (22 mg, 0.19 mmol, 0.2 eq), tosyl chloride (212 mg, 1.11 mmol, 1.2 eq) and NEt₃ (0.26 mL, 1.85 mmol, 2.0 eq) in CH₂Cl₂ (2 mL) was stirred at 25° C. for 16 h. The reaction was concentrated to dryness. The crude was purified by flash column chromatography eluting 15% EtOAc in PE. The desired fractions were concentrated to dryness under reduced pressure to give the title compound (151 mg, 0.6 mmol, 41% yield). MS: 270.1 [M+NH4]+

### Step 2: tert-Butyl (1S,5R,6r)-6-[4-[4-[[5-amino-1-[1-(1-bicyclo[1.1.1]pentanylmethyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl(1R,5S,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (50 mg, 0.07 mmol, 1.0 eq) in the DMF (2 mL) was added 1-bicyclo[1.1.1]pentanylmethyl 4-methylbenzenesulfonate (21 mg, 0.09 mmol, 1.2 eq) and K₂CO₃ (29 mg, 0.22 mmol, 3.0 eq). The reaction was stirred at 50° C. for 2 h before concentrating to dryness. The residue was taken up in EtOAc (20 ml) and the organics washed with H₂O (2×10 ml) then brine (10 ml). The organics were then separated and dried (Na₂SO₄) before concentration to dryness to give the title compound (45 mg, 0.06 mmol, 56% yield). MS: 716.3 [M−tBu+H]+

### Step 3: 5-Amino-N-[4-[4-[(1S,5R,6r)-3-azabicyclo[3.1.0]hexane-6-carbonyl]piperazine-1-carbonyl]-3-chlorophenyl]-1-[1-(1-bicyclo[1.1.1]pentanylmethyl)-3-(trifluoromethyl)pyrazol-4-yl]imidazole-4-carboxamide;2,2,2-trifluoroacetic acid

A solution of tert-butyl(1S,5R,6r)-6-[4-[4-[[5-amino-1-[1-(1-bicyclo[1.1.1]pentanylmethyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (40 mg, 0.05 mmol, 1.0 eq) and TFA (1.0 mL, 13 mmol, 2.6 eq) in CH₂Cl₂ (1 mL) was stirred at 25° C. for 1 h. The resulting solution was concentrated under reduced pressure. The crude product was purified by Prep-HPLC (CO₂-MeOH(DEA)). The product was freeze dried to get the title compound (12.6 mg, 0.02 mmol, 35% yield). MS: 672.3 [(M+H)+]

### Compound 396

### 5-Amino-N-[4-[4-[(1S,5R,6r)-3-azabicyclo[3.1.0]hexane-6-carbonyl]piperazine-1-carbonyl]-3-chlorophenyl]-1-[1-[(1-fluorocyclobutyl)methyl]-3-(trifluoromethyl)pyrazol-4-yl]imidazole-4-carboxamide;2,2,2-trifluoroacetic acid

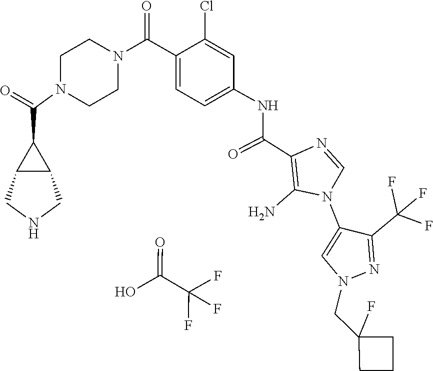 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2(F)CCC2)nc1C(F)(F)F ; O=C(O)C(F)(F)F]

### Step 1:(1-Fluorocyclobutyl)methyl 4-methylbenzenesulfonate

A solution of (1-fluorocyclobutyl)methanol (50 mg, 0.48 mmol, 1.0 eq), 4-dimethylaminopyridine (11 mg, 0.1 mmol, 0.2 eq), tosyl chloride (109 mg, 0.58 mmol, 1.2 eq) and NEt₃ (0.13 mL, 0.96 mmol, 2.0 eq) in CH₂Cl₂ (1 mL) was stirred at 25° C. for 16 h. The reaction was concentrated to dryness. The crude material was then purified by flash column chromatography eluting PE:EtOAc (10:1). The desired fractions were concentrated to the title compound (53 mg, 0.21 mmol, 27% yield). MS: 276.2 [M+NH4]+

### Step 2: tert-Butyl (1S,5R,6r)-6-[4-[4-[[5-amino-1-[1-[(1-fluorocyclobutyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl(1R,5S,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (60 mg, 0.09 mmol, 1.0 eq) in the DMF (2 mL) was added (1-fluorocyclobutyl)methyl 4-methylbenzenesulfonate (26 mg, 0.1 mmol, 1.2 eq) and K₂CO₃ (35 mg, 0.26 mmol, 3.0 eq). The reaction was stirred at 50° C. for 2 h before concentration to dryness. The residue was taken up in EtOAc (20 ml) and the organics were washed with H₂O (2×10 ml) and brine (10 ml). The organics were then separated and dried (Na₂SO₄) before concentration to dryness to afford the title compound (33 mg, 0.04 mmol, 33% yield). MS: 722.3 [M−tBu+H]]+

### Step 3: 5-Amino-N-[4-[4-[(1S,5R,6r)-3-azabicyclo[3.1.0]hexane-6-carbonyl]piperazine-1-carbonyl]-3-chlorophenyl]-1-[1-[(1-fluorocyclobutyl)methyl]-3-(trifluoromethyl)pyrazol-4-yl]imidazole-4-carboxamide;2,2,2-trifluoroacetic acid

A solution of tert-butyl(1S,5R,6r)-6-[4-[4-[[5-amino-1-[1-[(1-fluorocyclobutyl)methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (27 mg, 0.03 mmol, 1.0 eq) and trifluoroacetic acid (1.0 mL, 13 mmol, 4.2 eq) in CH₂Cl₂ (1 mL) was stirred at 25° C. for 1 h. The resulting solution was concentrated under reduced pressure. The crude product was purified by Prep-HPLC (Gemini-C18 150×21.2 mm, 5 μm, MeCN— H₂O (0.1% TFA), 15-40% B). The corresponding fractions were freeze dried to afford the title compound (7.4 mg, 0.01 mmol, 27% yield). MS: 678.3 [M+H]+

### Compound 397

### 5-Amino-N-[3-chloro-4-[4-[(2R,5R)-5-methylpyrrolidine-2-carbonyl]piperazine-1-carbonyl]phenyl]-1-[1-(cyclobutylmethyl)-3-(trifluoromethyl)pyrazol-4-yl]imidazole-4-carboxamide;2,2,2-trifluoroacetic acid

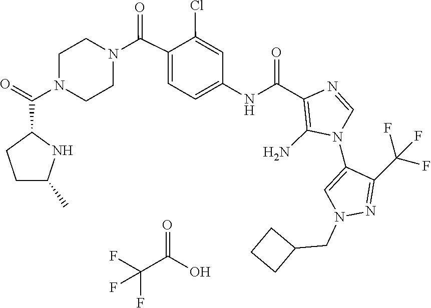 [SMILES: C[C@@H]1CC[C@H](C(=O)N2CCN(C(=O)c3ccc(NC(=O)c4ncn(-c5cn(CC6CCC6)nc5C(F)(F)F)c4N)cc3Cl)CC2)N1 ; O=C(O)C(F)(F)F]

### Step 1: Ethyl 5-amino-1-[3-(trifluoromethyl)-1H-pyrazol-4-yl]imidazole-4-carboxylate

A solution of ethyl 5-amino-1-[1-[(4-methoxyphenyl)methyl]-3-(trifluoromethyl)pyrazol-4-yl]imidazole-4-carboxylate (2.8 g, 6.84 mmol, 1.0 eq) in the trifluoroacetic acid (20 mL, 260 mmol, 38.0 eq) was stirred at 70° C. for 8 h. The reaction was concentrated to dryness to give the title compound (1.7 g, 5.88 mmol, 82% yield). MS: 290.0 [M+H]+

### Step 2: Ethyl 5-amino-1-[1-(cyclobutylmethyl)-3-(trifluoromethyl)pyrazol-4-yl]imidazole-4-carboxylate

To a solution of ethyl 5-amino-1-[3-(trifluoromethyl)-1H-pyrazol-4-yl]imidazole-4-carboxylate (1.65 g, 5.71 mmol, 1.0 eq) in DMF (20 mL) were added K₂CO₃ (2.37 g, 17.1 mmol, 3.0 eq) and (bromomethyl)cyclobutane (1.02 g, 6.85 mmol, 1.2 eq) under N₂ atmosphere. The vessel was sealed and the mixture was stirred at 25° C. for 15 h. The resulting solution was diluted with H₂O (100 ml), then extracted with EtOAc (3×100 mL). The organic layers were combined, washed with brine, dried and concentrated under reduced pressure. The crude product was purified by SFC separation (-chiralpak-IC, CO2-EtOH(DEA)) to afford the title compound (1838.0 mg, 5.14 mmol, 90% yield). MS: 358.1 [M+H]+

### Step 3: 5-Amino-1-[1-(cyclobutylmethyl)-3-(trifluoromethyl)pyrazol-4-yl]imidazole-4-carboxylic acid

A solution of ethyl 5-amino-1-[1-(cyclobutylmethyl)-3-(trifluoromethyl)pyrazol-4-yl]imidazole-4-carboxylate (850 mg, 2.38 mmol, 1.0 eq), NaOH (475 mg, 11.89 mmol, 5.0 eq) in i-PrOH (10 mL) and H₂O (5 mL) was stirred at 50° C. for 6 h. The solvent was removed under reduced pressure, and the residue was diluted with 100 ml of H₂O, then adjusted to pH 6˜7 with HCl (2M) and extracted with EtOAc (3×10 mL). The organic layers were combined and concentrated under reduced pressure to afford the title compoun (630 mg, 1.91 mmol, 80% yield). MS 330.1 [(M+H)+]

### Step 4: 5-Amino-1-[1-(cyclobutylmethyl)-3-(trifluoromethyl)pyrazol-4-yl]imidazole-4-carbonyl chloride

A solution of 5-amino-1-[1-(cyclobutylmethyl)-3-(trifluoromethyl)pyrazol-4-yl]imidazole-4-carboxylic acid (100 mg, 0.3 mmol, 1.0 eq) in thionyl chloride (2.0 mL, 27.57 mmol, 90.78 eq) was stirred at 25° C. for 1 h. The resulting solution was concentrated under reduced pressure to give the title compound (100 mg, 0.29 mmol, 94% yield). MS: 344.0 [M−Cl+OCH₃+H]+ (after MeOH quench)

### Step 5: tert-Butyl(2R,5R)-2-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]-5-methylpyrrolidine-1-carboxylate

A solution of tert-butyl(2R,5R)-2-[4-(4-amino-2-chloro-benzoyl)piperazine-1-carbonyl]-5-methyl-pyrrolidine-1-carboxylate (Intermediate 61, 64 mg, 0.14 mmol, 1.0 eq) in CH₂Cl₂ (3 mL) was added pyridine (227 mg, 2.88 mmol, 20.0 eq) and 5-amino-1-[1-(cyclobutylmethyl)-3-(trifluoromethyl)pyrazol-4-yl]imidazole-4-carbonyl chloride (50 mg, 0.14 mmol, 1.0 eq) was stirred at 25° C. for 5 h. The reaction was concentrated to dryness and the residue was taken up in CH₂Cl₂ (15 ml). This solution was washed with H₂O (2×15 ml) and brine (15 ml). The organics were then separated and dried (MgSO₄) before concentration to dryness. The crude was then purified by flash column chromatography eluting 12% MeOH in CH₂Cl₂. The desired fractions were concentrated to dryness under reduced pressure to afford the title compound (81 mg, 0.11 mmol, 66% yield) as yellow oil. MS: 762.2 [M+H]+

### Step 6: 5-Amino-N-[3-chloro-4-[4-[(2R,5R)-5-methylpyrrolidine-2-carbonyl]piperazine-1-carbonyl]phenyl]-1-[1-(cyclobutylmethyl)-3-(trifluoromethyl)pyrazol-4-yl]imidazole-4-carboxamide;2,2,2-trifluoroacetic acid

To a solution of tert-butyl(2R,5R)-2-[4-[4-[[5-amino-1-[1-(cyclobutylmethyl)-3-(trifluoromethyl)pyrazol-4-yl]imidazole-4-carbonyl]amino]-2-chlorobenzoyl]piperazine-1-carbonyl]-5-methylpyrrolidine-1-carboxylate (82 mg, 0.11 mmol, 1.0 eq) in CH₂Cl₂ (3 mL) was added trifluoroacetic acid (1.0 mL). The reaction was stirred at 25° C. for 4 h and then concentrated to dryness. The crude was purified by Prep-HPLC (Gemini-C18 150×21.2 mm, 5 μm, MeCN— H₂O (0.1% TFA), 20-40% B) to give the title compound (39.4 mg, 0.05 mmol, 55% yield). MS: 662.2 [M+H]+

### Compound 398

### 5-Amino-N-[3-chloro-4-[4-[(1R,5S,6r)-3-azabicyclo[3.1.0]hexane-6-carbonyl]piperazine-1-carbonyl]phenyl]-1-[1-(3,3-difluorocyclobutyl)-3-(trifluoromethyl)pyrazol-4-yl]imidazole-4-carboxamide;2,2,2-trifluoroacetic acid

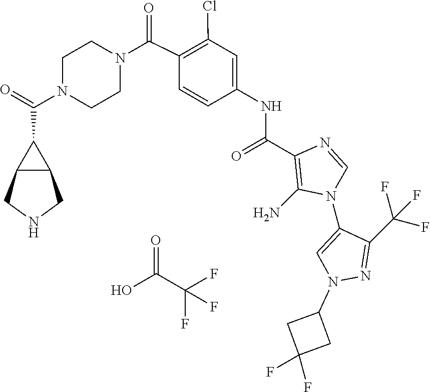 [SMILES: Nc1c(C(=O)Nc2ccc(C(=O)N3CCN(C(=O)[C@H]4[C@@H]5CNC[C@@H]54)CC3)c(Cl)c2)ncn1-c1cn(C2CC(F)(F)C2)nc1C(F)(F)F ; O=C(O)C(F)(F)F]

### Step 1: (3,3-difluorocyclobutyl) 4-methylbenzenesulfonate

A solution of 3,3-difluorocyclobutanol (100 mg, 0.93 mmol, 1.0 eq) in DCM (3 mL) was added p-toluenesulfonyl chloride (176 mg, 0.93 mmol, 1.0 eq) and pyridine (0.224 mL, 2.8 mmol, 3.0 eq) the reaction was stirred at 55° C. for 5 h The resulting solution was diluted with 10 ml of water, then extracted with EtOAc (20 ml) and the organics washed with 2×10 ml water then 1×10 ml brine. The combined organic layesr were dried dried (MgSO₄) before concentration to dryness. The crude material was then purified by flash column chromatography eluting 30% EtOAc in Isohexane. The desired fractions were concentrated to dryness in vacuo. to afford the title compound (170 mg, 0.65 mmol, 62.73% yield) as colorless oil. MS [M+H]+: 263.0

### Step 2: tert-Butyl (1R,5S,6r)-6-[4-[4-[[5-amino-1-[1-(3,3-difluorocyclobutyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

To a solution of tert-butyl(1S,5R,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (50 mg, 0.07 mmol, 1.0 eq) in DMF (3 mL) was added K₂CO₃ (29 mg, 0.22 mmol, 3.0 eq) and (3,3-difluorocyclobutyl) 4-methylbenzenesulfonate (56 mg, 0.22 mmol, 3.0 eq). The reaction was stirred at 50° C. for 12 h and then concentrated to dryness. The residue was taken up in EtOAc (20 ml) and the organics washed with H₂O (2×20 ml) then brine (20 ml). The organics were then separated and dried (MgSO₄) before concentration to dryness. The crude was then purified by flash column chromatography eluting 12% MeOH in CH₂Cl₂. The desired fractions were concentrated to dryness under reduced pressure.to give the title compound (40 mg, 0.05 mmol, 48% yield) as yellow oil. MS: 682.0 [M−Boc+H]+

### Step 3: 5-Amino-N-[3-chloro-4-[4-[(1R,5S,6r)-3-azabicyclo[3.1.0]hexane-6-carbonyl]piperazine-1-carbonyl]phenyl]-1-[1-(3,3-difluorocyclobutyl)-3-(trifluoromethyl)pyrazol-4-yl]imidazole-4-carboxamide;2,2,2-trifluoroacetic acid

To a solution of tert-butyl(1R,5S,6r)-6-[4-[4-[[5-amino-1-[1-(3,3-difluorocyclobutyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (35 mg, 0.04 mmol, 1.0 eq) in CH₂Cl₂ (3 mL) was added trifluoroacetic acid (1.0 mL, 13 mmol, 3.2 eq) the reaction was stirred at 25° C. for 3 h, The reaction was concentrated to dryness. The crude material was purified by Prep-HPLC (Gemini-C18 150×21.2 mm, 5 μm, MeCN— H₂O (0.1% TFA), 15-35% B) to give the title compound (4.1 mg, 0.01 mmol, 13% yield). MS: 682.0 [M+H]+

### Compound 399

### 5-Amino-N-[4-[4-[(1R,5S,6r)-3-azabicyclo[3.1.0]hexane-6-carbonyl]piperazine-1-carbonyl]-3-chlorophenyl]-1-[1-cyclobutyl-3-(trifluoromethyl)pyrazol-4-yl]imidazole-4-carboxamide; formic acid

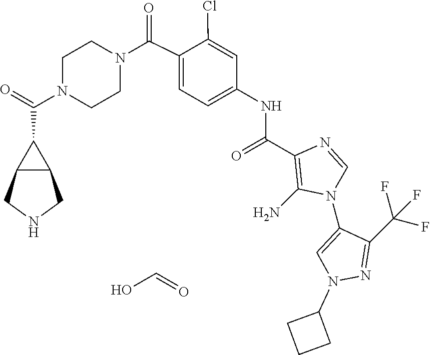 [SMILES: Nc1c(C(=O)Nc2ccc(C(=O)N3CCN(C(=O)[C@H]4[C@@H]5CNC[C@@H]54)CC3)c(Cl)c2)ncn1-c1cn(C2CCC2)nc1C(F)(F)F ; O=CO]

### Step 1: Cyclobutyl 4-methylbenzenesulfonate

A solution of cyclobutanol (100 mg, 1.39 mmol, 1.0 eq), NEt₃ (0.39 mL, 2.77 mmol, 2.0 eq), 4-dimethylaminopyridine (33 mg, 0.28 mmol, 0.2 eq) and p-toluenesulphonyl chloride (317.27 mg, 1.66 mmol, 1.2 eq) in CH₂Cl₂ (1 mL) was stirred at 25° C. for 15 h and then concentrated under reduced pressure. The residue applied on a silica gel column and eluted with EtOAc/PE (1/2) to give the title compound (240 mg, 1.06 mmol, 76% yield). MS: 227.0 [M+H]+

### Step 2: tert-Butyl (1S,5R,6r)-6-[4-[4-[[5-amino-1-[1-cyclobutyl-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of tert-butyl(1S,5R,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (70 mg, 0.1 mmol, 1.0 eq), cyclobutyl 4-methylbenzenesulfonate (34 mg, 0.15 mmol, 1.5 eq) and K₂CO₃ (41 mg, 0.3 mmol, 3.0 eq) in DMF (3 mL) was stirred at 50° C. for 16 h. The reaction was diluted with 50 ml of H₂O, then extracted with EtOAc (3×20 mL). The organic layers were combined, dried and concentrated under reduced pressure. The residue was applied on a silica gel column and eluted with CH₂Cl₂/MeOH (10/1) to give the title compound (65 mg, 0.09 mmol, 86% yield). MS: 690.2 [M−t−Bu+H]+

### Step 3: 5-Amino-N-[4-[4-[(1R,5S,6r)-3-azabicyclo[3.1.0]hexane-6-carbonyl]piperazine-1-carbonyl]-3-chlorophenyl]-1-[1-cyclobutyl-3-(trifluoromethyl)pyrazol-4-yl]imidazole-4-carboxamide; formic acid

A solution of tert-butyl(1S,5R,6r)-6-[4-[4-[[5-amino-1-[1-cyclobutyl-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (65 mg, 0.09 mmol, 1.0 eq), trifluoroacetic acid (1.13 mL, 14.7 mmol, 168 eq) in CH₂Cl₂ (4 mL) was stirred at 25° C. for 1 h. The resulting solution was concentrated under reduced pressure. The crude product was purified by Prep-HPLC (Gemini-C18 150×21.2 mm, 5 μm, MeCN— H₂O (0.1% FA), 10-40% B) to the title compound (9.8 mg, 0.01 mmol, 16% yield).

MS 646.2 [M+H]+

### Compound 400

### 5-amino-N-[4-[4-[(1S,5R,6r)-3-azabicyclo[3.1.0]hexane-6-carbonyl]piperazine-1-carbonyl]-3-chloro-phenyl]-1-[1-[cis((3-fluorocyclobutyl)methyl)]-3-(trifluoromethyl)pyrazol-4-yl]imidazole-4-carboxamide

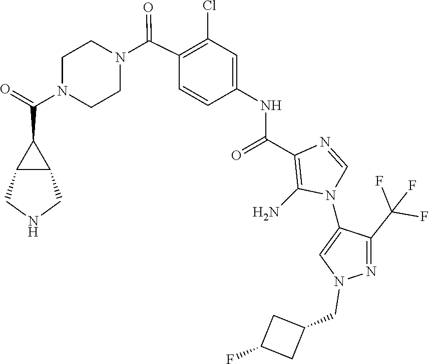 [SMILES: Nc1c(C(=O)Nc2ccc(C(=O)N3CCN(C(=O)[C@H]4[C@@H]5CNC[C@@H]54)CC3)c(Cl)c2)ncn1-c1cn(C[C@H]2C[C@@H](F)C2)nc1C(F)(F)F]

### Step 1: cis-(3-fluorocyclobutyl)methanol

A solution of 3-fluorocyclobutanecarboxylic acid ([123812-78-2], 200 mg, 1.69 mmol, 1.0 eq) in the Et2O (5 mL) was added lithium aluminum hydride (64 mg, 1.69 mmol, 1.0 eq) the reaction was stirred at 0° C. for 2 h. The reaction was then quenched by the addition of 0.06 ml of water and added 0.06 mL NaOH (15%) in water and 0.18 mL water. The solid was filtered and the filtrate was concentrated under vacuum to afford the title compound (150 mg, 85% yield) as a colorless liquid.

NMR (400 MHz,) δ 5.17-4.93 (m, 1H), 3.58 (dd, J=9.0, 5.8 Hz, 2H), 2.51-2.39 (m, 1H), 2.38-2.24 (m, 2H), 2.23-2.12 (m, 2H), 2.08 (s, 1H).

### Step 2: cis((3-fluorocyclobutyl)methyl) 4-methylbenzenesulfonate

A solution of cis-(3-fluorocyclobutyl)methanol (150 mg, 1.44 mmol, 1.0 eq), triethylamine (0.4 mL, 2.88 mmol, 2.0 eq), 4-dimethylaminopyridine (35.2 mg, 0.29 mmol, 0.2 eq) and p-toluenesulfonyl chloride (329 mg, 1.73 mmol, 1.2 eq) in DCM (3 mL) was stirred at 25° C. for 15 h. The mixture was concentrated and the residue purified by silica gel chromatography using EtOAc/PE (1/2) to afford cis((3-fluorocyclobutyl)methyl) 4-methylbenzenesulfonate (270 mg, 1.05 mmol, 72.55% yield). MS [M+Na]+: 281.0.

### Step 3: tert-butyl(1S,5R,6r)-6-[4-[4-[[5-amino-1-[1-[cis((3-fluorocyclobutyl)methyl)]-3-(trifluoromethyl)pyrazol-4-yl]imidazole-4-carbonyl]amino]-2-chloro-benzoyl]piperazine-1-carbonyl]-3-azabicyclo[3.1.0]hexane-3-carboxylate

To a solution of tert-butyl(1S,5R,6r)-6-[4-[4-[[5-amino-1-[3-(trifluoromethyl)-1H-pyrazol-4-yl]imidazole-4-carbonyl]amino]-2-chloro-benzoyl]piperazine-1-carbonyl]-3-azabicyclo[3.1.0]hexane-3-carboxylate (70 mg, 0.1 mmol, 1.0 eq), cis((3-fluorocyclobutyl)methyl) 4-methylbenzenesulfonate (39 mg, 0.15 mmol, 1.5 eq) and potassium carbonate (41 mg, 0.3 mmol, 3.0 eq) in DMF (3 mL) was stirred at 50° C. for 16 h. The resulting solution was diluted with 10 ml of water, then extracted with 3×10 mL of EtOAc. The organic layers were combined, dried and concentrated under vacuum. The residue was purified by silica gel chromatography and eluted with DCM/MeOH (10/1) to afford the title compound (60 mg, 0.08 mmol, 76.23% yield). MS [M−t−Bu+H]+: 722.2

### Step 4: 5-amino-N-[4-[4-[(1S,5R,6r)-3-azabicyclo[3.1.0]hexane-6-carbonyl]piperazine-1-carbonyl]-3-chloro-phenyl]-1-[1-[cis((3-fluorocyclobutyl)methyl)]-3-(trifluoromethyl)pyrazol-4-yl]imidazole-4-carboxamide

A solution of tert-butyl(1S,5R,6r)-6-[4-[4-[[5-amino-1-[1-[cis((3-fluorocyclobutyl)methyl)]-3-(trifluoromethyl)pyrazol-4-yl]imidazole-4-carbonyl]amino]-2-chloro-benzoyl]piperazine-1-carbonyl]-3-azabicyclo[3.1.0]hexane-3-carboxylate (60 mg, 0.08 mmol, 1.0 eq), trifluoroacetic acid (1.0 mL, 12.98 mmol, 168.35 eq) in DCM (4 mL) was stirred at 25° C. for 1 h. The resulting solution was concentrated under vacuum. The crude product was purified by SFC separation (-chiralpak-OJ, CO2-MeOH(DEA), 10-40) to afford the titlec compound (34.9 mg, 0.05 mmol, 66.76% yield). MS [M+H]+: 679.0

### Compound 401

### N-(4-(4-((1R,5S,6r)-3-azabicyclo[3.1.0]hexane-6-carbonyl)piperazine-1-carbonyl)-3-chlorophenyl)-5-amino-1-(1-(((1r,3r)-3-fluorocyclobutyl)methyl)-3-(trifluoromethyl)-1H-pyrazol-4-yl)-1H-imidazole-4-carboxamide 2,2,2-trifluoroacetate

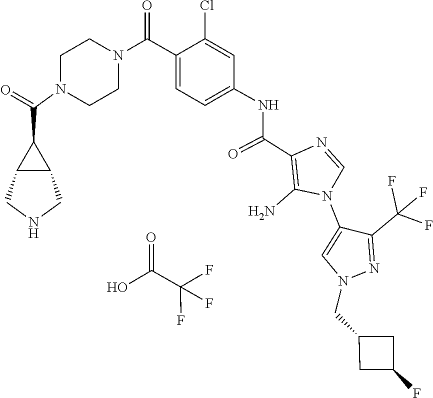 [SMILES: Nc1c(C(=O)Nc2ccc(C(=O)N3CCN(C(=O)[C@H]4[C@@H]5CNC[C@@H]54)CC3)c(Cl)c2)ncn1-c1cn(C[C@H]2C[C@H](F)C2)nc1C(F)(F)F ; O=C(O)C(F)(F)F]

### Step 1: trans-(3-fluorocyclobutyl)methanol

A solution of trans-3-fluorocyclobutanecarboxylic acid ([123812-79-3], 200 mg, 1.69 mmol, 1.0 eq) and lithium aluminum hydride (64 mg, 1.69 mmol, 1.0 eq) in Et2O (2 mL) was stirred at 0° C. for 2 h. The reaction was quenched by the addition of 0.3 mL water and added 0.3 mL NaOH (15%) in water and 0.9 mL water. The solid was filtered and the filtrate was concentrated under vacuum to afford the title compound (140 mg, 1.34 mmol, 75.44% yield) as a coloerless oil. 1H NMR (400 MHz, DMSO) δ 5.20-4.98 (m, 1H), 4.65 (t, J=5.3 Hz, 1H), 3.39-3.34 (m, 2H), 2.31 (qd, J=9.1, 5.3 Hz, 1H), 2.24-2.10 (m, 4H).

### Step 2: trans-(3-fluorocyclobutyl)methyl 4-methylbenzenesulfonate

A solution of trans-(3-fluorocyclobutyl)methanol (120 mg, 1.15 mmol, 1.0 eq), 4-dimethylaminopyridine (28 mg, 0.23 mmol, 0.2 eq), tosyl chloride (264 mg, 1.38 mmol, 1.2 eq) and triethylamine (0.32 mL, 2.31 mmol, 2.0 eq) in DCM (1 mL) was stirred at 25° C. for 16 h. The reaction was concentrated to dryness and the crude material was then purified by silica gel chromatography eluting PE:EA(4:1). The desired fractions were concentrated to afford the title compound (173 mg, 0.67 mmol, 54.04% yield). MS: 281.0 [M+Na]+

### Step 3: tert-butyl(1R,5S,6r)-6-(4-(4-(5-amino-1-(1-(((1r,3r)-3-fluorocyclobutyl)methyl)-3-(trifluoromethyl)-1H-pyrazol-4-yl)-1H-imidazole-4-carboxamido)-2-chlorobenzoyl)piperazine-1-carbonyl)-3-azabicyclo[3.1.0]hexane-3-carboxylate

A solution of tert-butyl(1R,5S,6r)-6-(4-(4-(5-amino-1-(3-(trifluoromethyl)-1H-pyrazol-4-yl)-1H-imidazole-4-carboxamido)-2-chlorobenzoyl)piperazine-1-carbonyl)-3-azabicyclo[3.1.0]hexane-3-carboxylate (60 mg, 0.09 mmol, 1.0 eq) in the DMF (2 mL) was added trans-(3-fluorocyclobutyl)methyl 4-methylbenzenesulfonate (26 mg, 0.1 mmol, 1.2 eq) and potassium carbonate (35 mg, 0.26 mmol, 3.0 eq) the reaction was stirred at 50° C. for 2 h. The reaction was concentrated to dryness and the residue was taken up in EtOAc (20 ml) and the organics washed with 2×10 ml water then 1×10 ml brine. The combined organic layers were dried (Na₂SO₄) before concentration to dryness to afford the title compund (52 mg, 0.07 mmol, 59.35% yield). MS: 678.3 [M−Boc+H]+

### Step 4: N-(4-(4-((1R,5S,6r)-3-azabicyclo[3.1.0]hexane-6-carbonyl)piperazine-1-carbonyl)-3-chlorophenyl)-5-amino-1-(1-(((1r,3r)-3-fluorocyclobutyl)methyl)-3-(trifluoromethyl)-1H-pyrazol-4-yl)-1H-imidazole-4-carboxamide 2,2,2-trifluoroacetate

A solution of tert-butyl(1S,5R,6r)-6-[4-[4-[[5-amino-1-[1-[trans-((3-fluorocyclobutyl)methyl)]-3-(trifluoromethyl)pyrazol-4-yl]imidazole-4-carbonyl]amino]-2-chloro-benzoyl]piperazine-1-carbonyl]-3-azabicyclo[3.1.0]hexane-3-carboxylate (42 mg, 0.05 mmol, 1.0 eq) and trifluoroacetic acid (1.0 mL) in DCM (1 mL) was stirred at 25° C. for 1 h. The resulting solution was concentrated under vacuum. The crude product was purified by Prep-HPLC (-Gemini-C18 150×21.2 mm, 5 um, ACN— H2O (0.1% TFA), 15-40) to afford the title compound (39.6 mg, 0.05 mmol, 90.41% yield). MS: 678.2 [M+H]+

### Compound 402

### 5-Amino-N-[3-chloro-4-[4-[(1S,5R,6r)-3-azabicyclo[3.1.0]hexane-6-carbonyl]piperazine-1-carbonyl]phenyl]-1-[1-[[1-(difluoromethyl)cyclopropyl]methyl]-3-(trifluoromethyl)pyrazol-4-yl]imidazole-4-carboxamide;2,2,2-trifluoroacetic acid

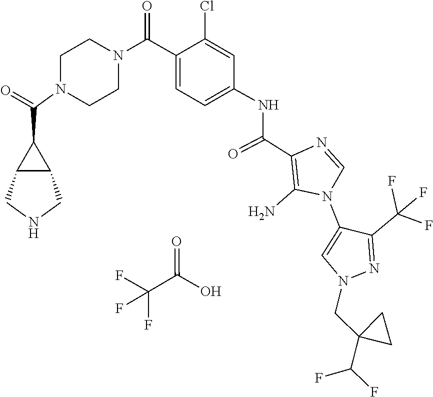 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC2(C(F)F)CC2)nc1C(F)(F)F ; O=C(O)C(F)(F)F]

### Step 1: [1-[[tert-butyl(diphenyl)silyl]oxymethyl]cyclopropyl]methanol

1,1-bis(hydroxymethyl)cyclopropane (1000 mg, 9.79 mmol, 1.0 eq) was dissolved in THF (30 mL), cooled to 0° C. and NaH (392 mg, 9.79 mmol, 1.0 eq) added. The resulting suspension was stirred at 25° C. for 1 h. Then tert-butylchlorodiphenylsilane (2691 mg, 9.79 mmol, 1.0 eq) added dropwise in 15 min. The reaction mixture was stirred at 25° C. for another 4 h, diluted with saturated aqueous NH₄Cl solution (80 mL) and extracted with CH₂Cl₂ (3×30 mL). The combined organic layers were washed with brine, dried (Na₂SO₄), filtered and concentrated under reduced pressure. The crude residue was purified by silica gel chromatography (PE:EtOAc=85:15) to afford the title compound (2640 mg, 7.75 mmol, 79.18% yield). MS: 363.2 [M+Na]+

### Step 2: tert-butyl-[[1-[[4-nitro-3-(trifluoromethyl)pyrazol-1-yl]methyl]cyclopropyl]methoxy]-diphenyl-silane

To a solution of 4-nitro-3-(trifluoromethyl)-1H-pyrazole (300 mg, 1.66 mmol, 1.0 eq) in toluene (10 mL) were added [1-[[tert-butyl(diphenyl)silyl]oxymethyl]cyclopropyl]methanol (677 mg, 1.99 mmol, 1.2 eq) and 2-(tributylphosphoranylidene)acetonitrile (799 mg, 3.31 mmol, 2.0 eq) under N₂ atmosphere. The resultant mixture was stirred at 110° C. for 8 h. The reaction was diluted with EtAOc (40 mL) and washed with water (25 mL×3), dried over Na₂SO₄ and concentrated under vacuo. The residue was purified by silica gel chromatography (PE:EA=90:10) to give the title compound (685 mg, 1.36 mmol, 78.82% yield). 1H NMR (400 MHz, CDCl3) δ 8.21 (s, 1H), 7.62 (dd, J=7.8, 1.4 Hz, 4H), 7.45-7.35 (m, 6H), 4.25 (s, 2H), 3.39 (s, 2H), 1.08 (s, 9H), 0.73 (t, J=5.6 Hz, 2H), 0.55 (t, J=5.6 Hz, 2H).

### Step 3: [1-[[4-nitro-3-(trifluoromethyl)pyrazol-1-yl]methyl]cyclopropyl]methanol

A solution of tert-butyl-[[1-[[4-nitro-3-(trifluoromethyl)pyrazol-1-yl]methyl]cyclopropyl]methoxy]-diphenyl-silane (685 mg, 1.36 mmol, 1.0 eq) in 2M hydrogen chloride in EtOH (4.72 mL, 9.45 mmol, 6.95 eq) was stirred at 50° C. for 15 h. The mixture was concentrated under vacuo and the residue was purified by silica gel chromatography (PE:EA=80:20) to afford the title compound (375 mg, 1.41 mmol, 98.76% yield). MS: 266.0 [M+H]+

### Step 4: 1-[[4-nitro-3-(trifluoromethyl)pyrazol-1-yl]methyl]cyclopropanecarbaldehyde

To a solution of [1-[[4-nitro-3-(trifluoromethyl)pyrazol-1-yl]methyl]cyclopropyl]methanol (325 mg, 1.23 mmol, 1.0 eq) in DCM (6 mL) was added dess-martin periodinane (728 mg, 1.72 mmol, 1.4 eq) at 0° C. under N₂ atmosphere. The resultant mixture was stirred at 0° C. for 5 h. The solid was filtered off and the solvent was removed under vacuo, the residue was diluted with EtOAc (20 mL) and washed with H₂O (10 mL×3), dried over Na₂SO₄ and concentrated under vacuo to afford the title compound (310 mg, 1.18 mmol, 89.39% yield) as a colorless gum, which was used without further purification. MS: 264.0 [M+H]+

### Step 5: 1-[[1-(difluoromethyl)cyclopropyl]methyl]-4-nitro-3-(trifluoromethyl)pyrazole

To a solution of 1-[[4-nitro-3-(trifluoromethyl)pyrazol-1-yl]methyl]cyclopropanecarbaldehyde (200 mg, 0.76 mmol, 1.0 eq) in bis(2-methoxyethyl)aminosulfur trifluoride (4.29 mL, 23.06 mmol, 30.35 eq) was added methanol (0.86 mL, 21.17 mmol, 27.86 eq) dropwise under N₂ atmosphere. The resultant mixture was stirred at 25° C. for 2 h, diluted with EtOAc (20 mL) and washed with water (15 mL×3), dried over Na₂SO₄ and concentrated under vacuo. The residue was purified by silica gel chromatography (PE:EA=97:3) to afford the title compound (130 mg, 60% yield). MS: 286.1 [M+H]+

### Step 6: 1-[[1-(Difluoromethyl)cyclopropyl]methyl]-3-(trifluoromethyl)pyrazol-4-amine

To a solution of 1-[[1-(difluoromethyl)cyclopropyl]methyl]-4-nitro-3-(trifluoromethyl)pyrazole (70 mg, 0.25 mmol, 1.0 eq) in EtOH (2 mL) were added Fe powder (68 mg, 1.23 mmol, 5.0 eq), NH₄Cl (131.3 mg, 2.45 mmol, 10.0 eq), and H₂O (0.4 mL) under N₂ atmosphere. The resultant mixture was stirred at 50° C. for 3 h. The solid was filtered out. The filtrate was concentrated under reduced pressure. The residue was diluted with H₂O, then extracted with EtOAc (3×40 mL). The organic layers were combined, washed with aqueous sodium carbonate and brine, dried and concentrated under reduced pressure to afford the title compound (60 mg, 0.24 mmol, 96% yield). MS: 256.0 [M+H]+

### Step 7: Ethyl 5-amino-1-[1-[[1-(difluoromethyl)cyclopropyl]methyl]-3-(trifluoromethyl)pyrazol-4-yl]imidazole-4-carboxylate

To a solution of ethyl 2-amino-2-cyano-acetate (50 mg, 0.39 mmol, 2.0 eq) in triethyl orthoformate (580 mg, 3.92 mmol, 20.0 eq) was added NEt₃ (79 mg, 0.78 mmol, 4.0 eq), the resultant mixture was stirred at 83° C. for 2 h. The solvent was removed under reduced pressure and the residue was diluted with MeCN (30 mL). Next 1-[[1-(difluoromethyl)cyclopropyl]methyl]-3-(trifluoromethyl)pyrazol-4-amine (50 mg, 0.2 mmol, 1.0 eq) was added and the resultant mixture was stirred at 25° C. for 15 h. The solvent was removed under reduced pressure. The residue was diluted with EtOAc (50 mL), washed with H₂O (3×20 mL), dried over Na₂SO₄, and concentrated under reduced pressure. The residue was purified by flash column chromatography (PE:EtOAc=55:45) to the title compound (35 mg, 0.09 mmol, 45% yield). MS: 394.0 [M+H]+

### Step 8: 5-Amino-1-[1-[[1-(difluoromethyl)cyclopropyl]methyl]-3-(Trifluoromethyl)pyrazol-4-yl]imidazole-4-carboxylic acid

A solution of ethyl 5-amino-1-[1-[[1-(difluoromethyl)cyclopropyl]methyl]-3-(trifluoromethyl)pyrazol-4-yl]imidazole-4-carboxylate (30 mg, 0.08 mmol, 1.0 eq), NaOH (15 mg, 0.38 mmol, 5.0 eq) in i-PrOH (1 mL) and H₂O (0.250 mL) was stirred at 50° C. for 6 h before concentrating under reduced pressure. The residue was diluted with 8 ml of H₂O, then adjusted to pH 6-7 with HCl (2 M) and extracted with EtOAc (3×8 mL). The organic layers were combined and concentrated under reduced pressure to afford the title compound (15 mg, 0.04 mmol, 54% yield). MS: 366.0 [M+H]+

### Step 9: tert-Butyl (1S,5R,6r)-6-[4-[4-[[5-Amino-1-[1-[[1-(difluoromethyl)cyclopropyl]methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of tert-butyl(1S,5R,6r)-6-[4-(4-amino-2-chlorobenzoyl)piperazine-1-carbonyl]-3-azabicyclo[3.1.0]hexane-3-carboxylate (Intermediate 22, 20 mg, 0.04 mmol, 1.0 eq) in thionyl chloride (5.3 mg, 0.04 mmol, 1.0 eq) was stirred at 25° C. for 1 h before concentrating under reduced pressure. To a solution of 5-amino-1-[1-[[1-(difluoromethyl)cyclopropyl]methyl]-3-(trifluoromethyl)pyrazol-4-yl]imidazole-4-carboxylic acid (17.9 mg, 0.05 mmol, 1.1 eq) and pyridine (35.19 mg, 0.45 mmol, 10.0 eq) in CH₂Cl₂ (2 mL) was added above solid in CH₂Cl₂ (2 mL). The resulting solution was concentrated under reduced pressure. The residue was applied on a silica gel column and eluted with MeOH/CH₂Cl₂ (1/20) to afford the title compound (15 mg, 0.02 mmol, 14% yield). MS: 695.9 [M+H−Boc]+

### Step 10: 5-Amino-N-[3-chloro-4-[4-[(1S,5R,6r)-3-azabicyclo[3.1.0]hexane-6-carbonyl]piperazine-1-carbonyl]phenyl]-1-[1-[[1-(difluoromethyl)cyclopropyl]methyl]-3-(trifluoromethyl)pyrazol-4-yl]imidazole-4-carboxamide;2,2,2-trifluoroacetic acid

A solution of tert-butyl(1S,5R,6r)-6-[4-[4-[[5-amino-1-[1-[[1-(difluoromethyl)cyclopropyl]methyl]-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (15 mg, 0.02 mmol, 1.0 eq) and trifluoroacetic acid (0.25 mL, 3.7 mmol, 18 eq) in CH₂Cl₂ (1 mL) was stirred at 25° C. for 1 h. The resulting solution was concentrated under reduced pressure. The crude product was purified by Prep-HPLC (Gemini-C18 150×21.2 mm, 5 μm, MeCN— H₂O (0.1% TFA), 15-50% B) to afford the title compound (3.3 mg, 0.0 mmol, 21% yield). MS: 695.9 [M+H]+

### Compound 403

### 5-Amino-N-[4-[4-[(1S,5R,6r)-3-azabicyclo[3.1.0]hexane-6-carbonyl]piperazine-1-carbonyl]-3-chlorophenyl]-1-[1-(2,3,3-trifluoroallyl)-3-(trifluoromethyl)pyrazol-4-yl]imidazole-4-carboxamide;2,2,2-trifluoroacetic acid

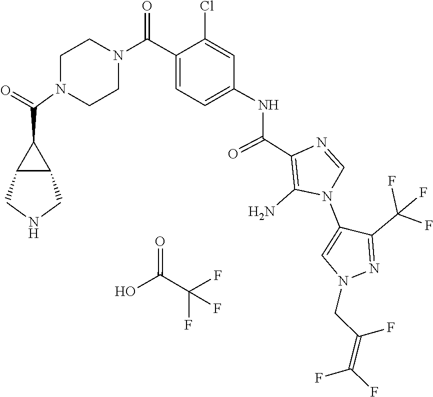 [SMILES: Nc1c(C(=O)Nc2ccc(C(=O)N3CCN(C(=O)[C@H]4[C@@H]5CNC[C@@H]54)CC3)c(Cl)c2)ncn1-c1cn(CC(F)=C(F)F)nc1C(F)(F)F ; O=C(O)C(F)(F)F]

### Step 1: 2,3,3-Trifluoroallyl methanesulfonate

A solution of 2,3,3-trifluoroprop-2-en-1-ol (200 mg, 1.78 mmol, 1.0 eq), methanesulphonyl chloride (0.28 mL, 3.57 mmol, 2.0 eq) and NEt₃ (0.75 mL, 5.35 mmol, 3.0 eq) in Et₂O (5 mL) was stirred at 20° C. for 2 h. The resulting solution was concentrated under reduced pressure to give the title compound (200 mg, 1.05 mmol, 53% yield), which was used without further purification.

### Step 2: tert-Butyl (1S,5R,6r)-6-[4-[4-[[5-Amino-1-[1-(2,3,3-trifluoroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate

A solution of 2,3,3-trifluoroallyl methanesulfonate (50 mg, 0.26 mmol, 1.0 eq), K₂CO₃ (109 mg, 0.79 mmol, 3.0 eq) and tert-butyl(1S,5R,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-3-azabicyclo[3.1.0]hexane-3-carboxylate (181 mg, 0.26 mmol, 1.0 eq) in DMF (3 mL) was stirred at 50° C. for 16 h. The resulting solution was extracted with EtOAc (3×10 mL). The organic layers were combined, washed with brine, dried and concentrated under reduced pressure. The residue was applied on a silica gel column and eluted with CH₂Cl₂/MeOH (25:1) to the title compound (150 mg, 0.19 mmol, 73% yield). MS: 730 [M+H−tBu]+

### Step 3: 5-Amino-N-[4-[4-[(1S,5R,6r)-3-azabicyclo[3.1.0]hexane-6-carbonyl]piperazine-1-carbonyl]-3-chlorophenyl]-1-[1-(2,3,3-trifluoroallyl)-3-(trifluoromethyl)pyrazol-4-yl]imidazole-4-carboxamide;2,2,2-trifluoroacetic acid

A solution of tert-butyl(1S,5R,6r)-6-[4-[4-[[5-amino-1-[1-(2,3,3-trifluoroallyl)-3-(trifluoromethyl)pyrazol-4-yl]imidazole-4-carbonyl]amino]-2-chlorobenzoyl]piperazine-1-carbonyl]-3-azabicyclo[3.1.0]hexane-3-carboxylate (100 mg, 0.13 mmol, 1.0 eq), trifluoroacetic acid (1.0 mL, 13 mmol, 100 eq) in CH₂Cl₂ (4 mL) was stirred at 20° C. for 2 h before concentration under reduced pressure. The residue was purified by Prep-HPLC (Gemini-C18 150×19 mm, 5 μm, MeCN— H₂O (0.1% TFA), 35-45% B) to afford the title compound (11.3 mg, 0.01 mmol, 13% yield). MS: 686 [M+H]+

### Compound 404

### N-[4-[4-[(3aR,6aS)-1,2,3,3a,4,5,6,6a-Octahydrocyclopenta[c]pyrrole-5-carbonyl]piperazine-1-carbonyl]-3-chlorophenyl]-5-amino-1-[1-(2-fluoroallyl)-3-(trifluoromethyl)pyrazol-4-yl]imidazole-4-carboxamide;2,2,2-trifluoroacetic acid

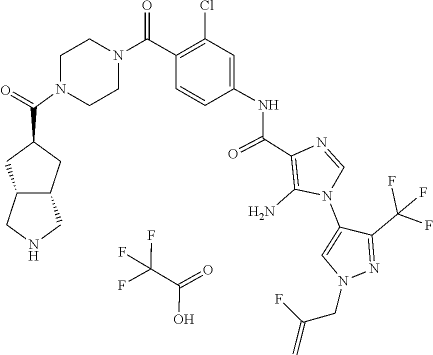 [SMILES: C=C(F)Cn1cc(-n2cnc(C(=O)Nc3ccc(C(=O)N4CCN(C(=O)[C@@H]5C[C@H]6CNC[C@H]6C5)CC4)c(Cl)c3)c2N)c(C(F)(F)F)n1 ; O=C(O)C(F)(F)F]

### Step 1: 5-Amino-1-[1-(2-fluoroallyl)-3-(trifluoromethyl)pyrazol-4-yl]imidazole-4-carbonyl chloride

A solution of 5-amino-1-[1-(2-fluoroallyl)-3-(trifluoromethyl)pyrazol-4-yl]imidazole-4-carboxylic acid (Intermediate 51, 80 mg, 0.25 mmol, 1.0 eq) in thionyl chloride (265.45 mL, 3659.2 mmol, 14600 eq) was stirred at 25° C. for 1 h. The reaction was concentrated under reduced pressure to afford the title compound (80.0 mg, 0.24 mmol, 71% yield). MS: 335.0 [(M−Cl+OCH3)+H] (after MeOH quench)

### Step 2: tert-Butyl (3aR,6aS)-5-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-3,3a,4,5,6,6a-hexahydro-1H-cyclopenta[c]pyrrole-2-carboxylate

To a solution of tert-butyl(3aR,6aS)-5-(4-(4-amino-2-chlorobenzoyl)piperazine-1-carbonyl) hexahydrocyclopenta[c]pyrrole-2 (1H)-carboxylate (Intermediate 56, 91 mg, 0.19 mmol, 0.7 eq) in CH₂Cl₂ (3 mL) were added pyridine (0.45 mL, 5.51 mmol, 20.0 eq) and 5-amino-1-[1-(2-fluoroallyl)-3-(trifluoromethyl)pyrazol-4-yl]imidazole-4-carbonyl chloride (93 mg, 0.28 mmol, 1.0 eq) in order under N₂ atmosphere. The resultant mixture was stirred at 25° C. for 1 h, diluted with CH₂Cl₂ (20 mL), washed with H₂O (3×15 mL), dried over Na₂SO₄, and concentrated under reduced pressure. The residue was purified by flash column chromatography (CH₂Cl₂:MeOH=92:8) to afford the title compound (214 mg, 0.27 mmol, 80% yield). MS: 677.9 [M−Boc+H]+

### Step 3: N-[4-[4-[(3aR,6aS)-1,2,3,3a,4,5,6,6a-Octahydrocyclopenta[c]pyrrole-5-carbonyl]piperazine-1-carbonyl]-3-chlorophenyl]-5-amino-1-[1-(2-fluoroallyl)-3-(trifluoromethyl)pyrazol-4-yl]imidazole-4-carboxamide;2,2,2-trifluoroacetic acid

To a solution of tert-butyl(3aR,6aS)-5-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-3,3a,4,5,6,6a-hexahydro-1H-cyclopenta[c]pyrrole-2-carboxylate (214 mg, 0.27 mmol, 1.0 eq) in CH₂Cl₂ (2 mL) was added trifluoroacetic acid (1.0 mL, 13 mmol, 47 eq) under N₂ atmosphere. The resulting mixture was stirred at 25° C. for 1 h and then concentrated under reduced pressure. The residue was purified by prep-HPLC [Gemini-C18 150×21.2 mm, 5 μm, MeCN— H₂O (0.1% TFA), 25-35% B] to afford the title compound (80.1 mg, 0.1 mmol, 36.04% yield). MS: 678.3 [M+H]+

### Compound 405

### 5-Amino-N-[3-chloro-4-[4-[1-(3-hydroxypropyl)piperidine-4-carbonyl]piperazine-1-carbonyl]phenyl]-1-[1-(2-fluoroallyl)-3-(trifluoromethyl)pyrazol-4-yl]imidazole-4-carboxamide;2,2,2-trifluoroacetic acid

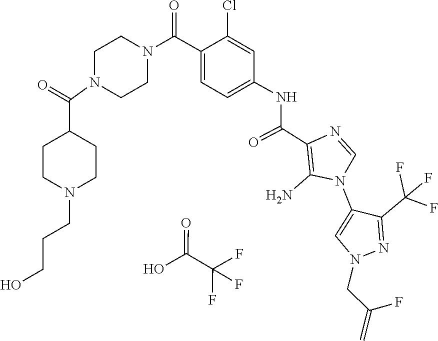 [SMILES: C=C(F)Cn1cc(-n2cnc(C(=O)Nc3ccc(C(=O)N4CCN(C(=O)C5CCN(CCCO)CC5)CC4)c(Cl)c3)c2N)c(C(F)(F)F)n1 ; O=C(O)C(F)(F)F]

### Step 1: 2-Fluoroallyl 4-methylbenzenesulfonate

A solution of 2-fluoroprop-2-en-1-ol (150 mg, 1.97 mmol, 1.0 eq) in CH₂Cl₂ (5 mL) was added p-toluenesulphonyl chloride (375 mg, 1.97 mmol, 1.0 eq), NEt₃ (0.82 mL, 5.92 mmol, 3.0 eq) and 4-dimethylaminopyridine (240.9 mg, 1.97 mmol, 1.0 eq) the reaction was stirred at 25° C. for 4 h The reaction was concentrated to dryness to give a crude product The crude was purified by flash column chromatography eluting 10% EtOAc in isohexane. The desired fractions were concentrated to dryness under reduced pressure. to afford the title compound (110 mg, 0.48 mmol, 22% yield) as colorless oil. MS: 231.0 [M+H]+

### Step 2: tert-Butyl 4-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]piperidine-1-carboxylate

To a solution of tert-butyl 4-[4-[4-[[5-amino-1-[3-(trifluoromethyl)-1H-pyrazol-4-yl]imidazole-4-carbonyl]amino]-2-chlorobenzoyl]piperazine-1-carbonyl]piperidine-1-carboxylate (Intermediate 81, 210 mg, 0.3 mmol, 1.0 eq), 2-fluoroallyl 4-methylbenzenesulfonate (209 mg, 0.91 mmol, 3.0 eq) and K₂CO₃ (125 mg, 0.91 mmol, 3.0 eq) in DMF (3 mL) was stirred at 50° C. for 16 h. The resulting solution was diluted with 10 ml of H₂O, then extracted with EtOAc (3×10 mL). The organic layers were combined, dried and concentrated under reduced pressure. The residue was applied on a silica gel column and eluted with CH₂Cl₂/MeOH (10/1) to affor the title compound (180 mg, 0.24 mmol, 79% yield). MS: 652.1 [M−Boc+H]+

### Step 3: 5-Amino-N-[3-chloro-4-[4-(piperidine-4-carbonyl)piperazine-1-carbonyl]phenyl]-1-[1-(2-fluoroallyl)-3-(trifluoromethyl)pyrazol-4-yl]imidazole-4-carboxamide

A solution of tert-butyl 4-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]piperidine-1-carboxylate (120 mg, 0.16 mmol, 1.0 eq), trifluoroacetic acid (1.49 mL, 19.3 mmol, 121 eq) in CH₂Cl₂ (4 mL) was stirred at 25° C. for 1 h. The resulting solution was concentrated under reduced pressure to afford the title compound (90 mg, 0.14 mmol, 86% yield). MS: 652.1 [M+H]+

### Step 4: 5-Amino-N-[3-chloro-4-[4-[1-(3-hydroxypropyl)piperidine-4-carbonyl]piperazine-1-carbonyl]phenyl]-1-[1-(2-fluoroallyl)-3-(trifluoromethyl)pyrazol-4-yl]imidazole-4-carboxamide;2,2,2-trifluoroacetic acid

A solution of 5-amino-N-[3-chloro-4-[4-(piperidine-4-carbonyl)piperazine-1-carbonyl]phenyl]-1-[1-(2-fluoroallyl)-3-(trifluoromethyl)pyrazol-4-yl]imidazole-4-carboxamide (45 mg, 0.07 mmol, 1.0 eq), 3-hydroxypropanal (15 mg, 0.21 mmol, 3.0 eq), NaOAc (16 mg, 0.21 mmol, 3.0 eq), sodium cyanoborohydride (21 mg, 0.35 mmol, 5.0 eq) in MeOH (3 mL) was stirred at 25° C. for 1 h before concentration under reduced pressure. The crude product was purified by Prep-HPLC (Gemini-C18 150×21.2 mm, 5 μm, MeCN— H₂O (0.1% TFA), 20-50) to afford the title compound (17.3 mg, 0.02 mmol, 30% yield).

MS: 710.1 [M+H]+

### Compound 406

### 5-Amino-N-[3-chloro-4-[4-(3-methyl-5,6-dihydro-4H-pyrrolo[3,4-d]imidazole-2-carbonyl)piperazine-1-carbonyl]phenyl]-1-[1-(2-fluoroallyl)-3-(trifluoromethyl)pyrazol-4-yl]imidazole-4-carboxamide;2,2,2-trifluoroacetic acid

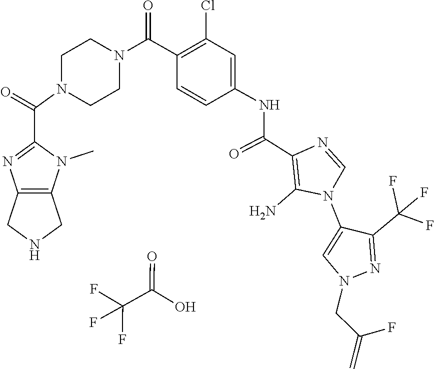 [SMILES: C=C(F)Cn1cc(-n2cnc(C(=O)Nc3ccc(C(=O)N4CCN(C(=O)c5nc6c(n5C)CNC6)CC4)c(Cl)c3)c2N)c(C(F)(F)F)n1 ; O=C(O)C(F)(F)F]

### Step 1: 5-tert-Butoxycarbonyl-3-methyl-4,6-dihydropyrrolo[3,4-d]imidazole-2-carboxylic acid

O5-tert-butyl O2-ethyl 3-methyl-4,6-dihydropyrrolo[3,4-d]imidazole-2,5-dicarboxylate ([1846626-71-8], 400.0 mg, 1.35 mmol, 1.0 eq) was dissolved in EtOH (10 mL) A solution of LiOH·H₂O (284 mg, 6.77 mmol, 5.0 eq) in H₂O (2 mL) was added dropwise at 25° C. for 3 h. The solvent was evaporated. The residue was diluted with H₂O (10 mL), and the solution was subjected to extraction with EtOAc (2×100 mL). The aqueous layer was acidified to pH 5 with HCl (2M), and the product was collected by filtration to afford the title compound (290 mg, 1.09 mmol, 80% yield). MS: 268.1 [M+H]+

### Step 2: tert-Butyl 2-[4-[4-[[5-amino-1-[3-(trifluoromethyl)-1H-pyrazol-4-yl]imidazole-4-carbonyl]amino]-2-chlorobenzoyl]piperazine-1-carbonyl]-3-methyl-4,6-dihydropyrrolo[3,4-d]imidazole-5-carboxylate

To a solution of 5-amino-N-[3-chloro-4-(piperazine-1-carbonyl)phenyl]-1-[3-(trifluoromethyl)-1H-pyrazol-4-yl]imidazole-4-carboxamide (Intermediate 77, 180 mg, 0.37 mmol, 1.0 eq) in DMF (3 mL) was added 5-tert-butoxycarbonyl-3-methyl-4,6-dihydropyrrolo[3,4-d]imidazole-2-carboxylic acid (100 mg, 0.37 mmol, 1.0 eq), N,N-diisopropylethylamine (144 mg, 1.12 mmol, 3.0 eq) and propylphosphonic anhydride solution (595.2 mg, 1.87 mmol, 5.0 eq). The reaction was stirred at 25° C. for 16 h before concentration to dryness. The residue was taken up in EtOAc (30 ml) and the organics washed with H₂O (2×10 ml) then brine (10 ml). The organics were then separated and dried (MgSO₄) before concentration to dryness. The crude was then purified by flash column chromatography eluting 5% MeOH in CH₂Cl₂. The desired fractions were concentrated to dryness under reduced pressure. to afford the title compound (106 mg, 0.14 mmol, 34% yield). MS [M+H]+: 731.9 RT=1.177, LC/MS 97.57% (254 nm); 89.06% (214 nm)

### Step 3: tert-Butyl 2-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-3-methyl-4,6-dihydropyrrolo[3,4-d]imidazole-5-carboxylate

To a solution of tert-butyl 2-[4-[4-[[5-amino-1-[3-(trifluoromethyl)-1H-pyrazol-4-yl]imidazole-4-carbonyl]amino]-2-chlorobenzoyl]piperazine-1-carbonyl]-3-methyl-4,6-dihydropyrrolo[3,4-d]imidazole-5-carboxylate (100 mg, 0.14 mmol, 1.0 eq) in DMF (3 mL) was added 2-fluoroallyl 4-methylbenzenesulfonate (31 mg, 0.14 mmol, 1.0 eq) and K₂CO₃ (56 mg, 0.41 mmol, 3.0 eq). The reaction was stirred at 25° C. for 16 h before concentration to dryness. The residue was taken up in EtOAc (20 ml), and the organics were washed with H₂O (2×20 ml) and brine (20 ml). The organics were dried (MgSO₄) before concentration to dryness. The crude was then purified by flash column chromatography eluting 5% MeOH in CH₂Cl₂. The desired fractions were concentrated to dryness under reduced pressure to afford the title compound (65 mg, 0.08 mmol, 50% yield). MS: 790.0 [M+H]+

### Step 5: 5-Amino-N-[3-chloro-4-[4-(3-methyl-5,6-dihydro-4H-pyrrolo[3,4-d]imidazole-2-carbonyl)piperazine-1-carbonyl]phenyl]-1-[1-(2-fluoroallyl)-3-(trifluoromethyl)pyrazol-4-yl]imidazole-4-carboxamide;2,2,2-trifluoroacetic acid

To a solution of tert-butyl 2-[4-[4-[[5-amino-1-[1-(2-fluoroallyl)-3-(trifluoromethyl)pyrazol-4-yl]imidazole-4-carbonyl]amino]-2-chlorobenzoyl]piperazine-1-carbonyl]-3-methyl-4,6-dihydropyrrolo[3,4-d]imidazole-5-carboxylate (60 mg, 0.08 mmol, 1.0 eq) in CH₂Cl₂ (3 mL) was added trifluoroacetic acid (1.0 mL, 0.08 mmol, 1.0 eq). The reaction was stirred at 25° C. for 2 h before concentration to dryness. The crude was purified by Prep-HPLC (Gemini-C18 150×21.2 mm, 5 μm, MeCN— H₂O (0.1% TFA), 15-40) to afford the title compound (36.1 mg, 0.04 mmol, 58% yield). MS: 689.9 [M+H]+

### Compound 407

### 5-Amino-N-[3-chloro-4-[4-(piperazine-1-carbonyl)piperidine-1-carbonyl]phenyl]-1-[1-(1,1-difluoroallyl)-3-(trifluoromethyl)pyrazol-4-yl]imidazole-4-carboxamide;2,2,2-trifluoroacetic acid

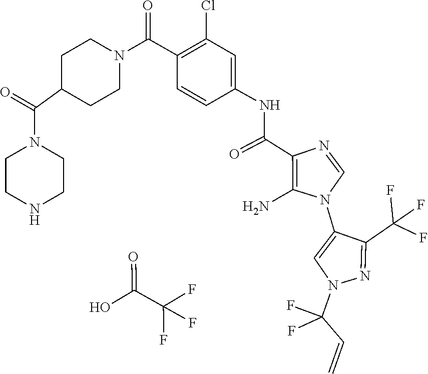 [SMILES: C=CC(F)(F)n1cc(-n2cnc(C(=O)Nc3ccc(C(=O)N4CCC(C(=O)N5CCNCC5)CC4)c(Cl)c3)c2N)c(C(F)(F)F)n1 ; O=C(O)C(F)(F)F]

### Step 1: tert-butyl 4-[1-[4-[[5-amino-1-[3-(trifluoromethyl)-1H-pyrazol-4-yl]imidazole-4-carbonyl]amino]-2-chloro-benzoyl]piperidine-4-carbonyl]piperazine-1-carboxylate

To a solution of 1-[4-[[5-amino-1-[3-(trifluoromethyl)-1H-pyrazol-4-yl]imidazole-4-carbonyl]amino]-2-chloro-benzoyl]piperidine-4-carboxylic acid (Intermediate 82, 490 mg, 0.93 mmol, 1.0 eq) in DCM (2 mL) were added 1-BOC-piperazine (190.9 mg, 1.02 mmol, 1.1 eq), 50% 1,3,5,2,4,6-trioxatriphosphorinane (1342 mg, 1.86 mmol, 2.0 eq) and diisopropylethylamine (361 mg, 2.8 mmol, 3.0 eq). The resultant mixture was stirred at 25° C. for 1 h. The solvent was removed under vacuo. The residue was diluted with EtOAc (100 mL) and washed with H₂O (60 mL×4), dried over Na₂SO₄ and concentrated under vacuo and the residue was purified by flash column chromatography (PE:EtOAc=80:20) to afford the title compound (480 mg, 0.69 mmol, 52.69% yield). MS: 693.9 [M+H]+

### Step 2: tert-Butyl 4-[1-[4-[[5-amino-1-[1-(1,1-difluoroallyl)-3-(trifluoromethyl)pyrazol-4-yl]imidazole-4-carbonyl]amino]-2-chlorobenzoyl]piperidine-4-carbonyl]piperazine-1-carboxylate

To a solution of tert-butyl 4-[1-[4-[[5-amino-1-[3-(trifluoromethyl)-1H-pyrazol-4-yl]imidazole-4-carbonyl]amino]-2-chloro-benzoyl]piperidine-4-carbonyl]piperazine-1-carboxylate (437.8 mg, 0.63 mmol, 1.1 eq) in DMF (2 mL) was added 3-bromo-3,3-difluoro-prop-1-ene (90 mg, 0.57 mmol, 1.0 eq) and caesium carbonate (224 mg, 0.69 mmol, 1.2 eq). The reaction was stirred at 50° C. for 2 h before concentration under reduced pressure. The crude product was purified by SFC (chiralpak-AD,CO2-ETOH(EtOAc)). The product was freeze-dried to afford the title compound (20 mg, 0.03 mmol, 4% yield). MS: 770.1 [M+H]+

### Step 3: 5-Amino-N-[3-chloro-4-[4-(piperazine-1-carbonyl)piperidine-1-carbonyl]phenyl]-1-[1-(1,1-difluoroallyl)-3-(trifluoromethyl)pyrazol-4-yl]imidazole-4-carboxamide;2,2,2-trifluoroacetic acid

A solution of tert-butyl 4-[1-[4-[[5-amino-1-[1-(1,1-difluoroallyl)-3-(trifluoromethyl)pyrazol-4-yl]imidazole-4-carbonyl]amino]-2-chlorobenzoyl]piperidine-4-carbonyl]piperazine-1-carboxylate (20 mg, 0.03 mmol, 1.0 eq) and trifluoroacetic acid (2 mg, 0.03 mmol, 1.0 eq) in CH₂Cl₂ (1 mL) was stirred at 25° C. for 1 h. The resulting solution was concentrated under reduced pressure. The crude product was purified by Prep-HPLC (Gemini-C18 150×21.2 mm, 5 μm, MeCN— H₂O (0.1% TFA), 15-45) to afford the title compound (6.2 mg, 0.01 mmol, 30% yield). MS: 670.0 [M+H]+

### Compound 408

### 5-Amino-N-[3-chloro-4-[4-[(1S,5R,6r)-6-fluoro-3-azabicyclo[3.1.0]hexane-6-carbonyl]piperazine-1-carbonyl]phenyl]-1-[1-(cyclopropylmethyl)-3-(trifluoromethyl)pyrazol-4-yl]imidazole-4-carboxamide;2,2,2-trifluoroacetic acid

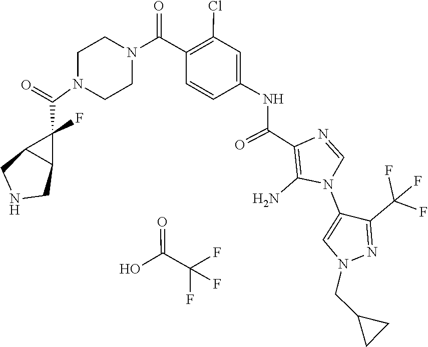 [SMILES: Nc1c(C(=O)Nc2ccc(C(=O)N3CCN(C(=O)[C@@]4(F)[C@@H]5CNC[C@@H]54)CC3)c(Cl)c2)ncn1-c1cn(CC2CC2)nc1C(F)(F)F ; O=C(O)C(F)(F)F]

### Step 1:(1R,5S,6r)-3-(tert-butoxycarbonyl)-6-fluoro-3-azabicyclo[3.1.0]hexane-6-carboxylic acid

A stirred solution of 3-(tert-butyl) 6-ethyl (1R,5S,6r)-6-fluoro-3-azabicyclo[3.1.0]hexane-3,6-dicarboxylate ([2414568-88-8], 30.0 mg, 0.11 mmol, 1.0 eq) in THF (1 mL)/water (0.500 mL) was added lithium;hydroxide;hydrate (9.21 mg, 0.22 mmol, 2.0 eq) at 20° C. The solution was stirred at 20° C. for 2 h. The reaction mixture was concentrated in vacuum to afford the title compouind (20.0 mg, 0.08 mmol, 74.29% yield), which was used without further purification. MS 1.190.1, [M−tBu+H]+, ESI+

### Step 2: tert-butyl(1S,5R,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-6-fluoro-3-azabicyclo[3.1.0]hexane-3-carboxylate

A solution of (1R,5S,6r)-3-(tert-butoxycarbonyl)-6-fluoro-3-azabicyclo[3.1.0]hexane-6-carboxylic acid (508 mg, 2.07 mmol, 1.0 eq) in DMF (12 mL) was added 2-(7-azabenzotriazol-1-yl)-N,N,N′,N′-tetramethyluronium hexafluorophosphate (1575 mg, 4.14 mmol, 2.0 eq), triethylamine (628.7 mg, 6.21 mmol, 3.0 eq) and 5-amino-N-[3-chloro-4-(piperazine-1-carbonyl)phenyl]-1-[3-(trifluoromethyl)-1H-pyrazol-4-yl]imidazole-4-carboxamide (1000.0 mg, 2.07 mmol, 1.0 eq) the reaction was stirred at 25° C. for 2 h The reaction was concentrated to dryness and the residue was taken up in EtOAc (30 ml) and the organics washed with 2×30 ml water then 1×30 ml saturated brine solution. The organics were then separated and dried (MgSO₄) before concentration to dryness. The crude was then purified by silica gel chromatography eluting 60% EtOAc in Isohexane. The desired fractions were concentrated to dryness in vacuo.to afford the title compound (730.0 mg, 1.03 mmol, 49.64% yield). MS: 654.0 [M−tBu+H]+

### Step 3: tert-Butyl (1R,5S,6r)-6-[4-[4-[[5-amino-1-[1-(cyclopropylmethyl)-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate

A solution of tert-butyl(1S,5R,6r)-6-[4-[4-[[5-amino-1-[3-(trifluoromethyl)-1H-pyrazol-4-yl]imidazole-4-carbonyl]amino]-2-chlorobenzoyl]piperazine-1-carbonyl]-6-fluoro-3-azabicyclo[3.1.0]hexane-3-carboxylate (80 mg, 0.11 mmol, 1.0 eq), (bromomethyl)cyclopropane (0.02 mL, 0.17 mmol, 1.5 eq) and K₂CO₃ (46 mg, 0.34 mmol, 3.0 eq) in DMF (3 mL) was stirred at 25° C. for 2 h. The reaction was diluted with H₂O (10 mL) and then extracted with EtOAc (3×10 mL). The organic layers were combined, dried and concentrated under reduced pressure. The residue was applied on a silica gel column and eluted with CH₂Cl₂/MeOH (10/1) to give the title compound (65.0 mg, 0.09 mmol, 75% yield). MS: 708.2 [M+H]+

### Step 4: 5-Amino-N-[3-chloro-4-[4-[(1S,5R,6r)-6-fluoro-3-azabicyclo[3.1.0]hexane-6-carbonyl]piperazine-1-carbonyl]phenyl]-1-[1-(cyclopropylmethyl)-3-(trifluoromethyl)pyrazol-4-yl]imidazole-4-carboxamide;2,2,2-trifluoroacetic acid

A solution of tert-butyl(1S,5R,6r)-6-[4-[4-[[5-amino-1-[1-(cyclopropylmethyl)-3-(trifluoromethyl)pyrazol-4-yl]imidazole-4-carbonyl]amino]-2-chlorobenzoyl]piperazine-1-carbonyl]-6-fluoro-3-azabicyclo[3.1.0]hexane-3-carboxylate (65 mg, 0.09 mmol, 1.0 eq), trifluoroacetic acid (0.72 mL, 9.33 mmol, 109.66 eq) in CH₂Cl₂ (4 mL) was stirred at 25° C. for 1 h. The reaction was concentrated under reduced pressure. The crude product was purified by Prep-HPLC (-Gemini-C18 150×21.2 mm, 5 μm, MeCN— H₂O (0.1% TFA), 30-60) to afford the title compound (41.2 mg, 0.05 mmol, 62% yield). MS: 664.1 [M+H]+

### Assay Procedures

Antimicrobial susceptibility testing: 90% Growth Inhibitory Concentration (IC₉₀) determination

The in vitro antimicrobial activity of the compounds was determined according to the following procedure:

The assay used a 10-point dilution series of test compounds to measure quantitatively the in vitro activity of the compounds against Acinetobacter baumannii ATCC17961.

Stock solutions of compounds in DMSO were serially diluted in two-fold steps and aliquots of maximally 500 nL were transferred to 384-well microtiter plates (e.g. for a final range from 10 to 0.0195 μM). 50 μL of a bacterial suspension in Iso-Sensitest broth of ˜5×10⁵ CFU/mL were added per well and plates were incubated at 37° C.

Bacterial cell growth was determined with the measurement of optical density at λ=600 nm each 20 min over a time course of 16 h. Growth inhibition was calculated during the logarithmic growth of the bacterial cells with determination of the concentration inhibiting 50% (IC₅₀) and 90% (IC₉₀) of the growth.

Table 2 provides the 90% growth inhibitory concentrations (IC₉₀) in micromoles per liter of the compounds of present invention obtained against the strain Acinetobacter baumannii ATCC17961.

TABLE 2

| | ATCC |
|---|---|
| Cmpd | 17961 |
| No | IC90 (μM) |
| 1 | 0.102 |
| 2 | 0.0967 |
| 3 | 0.0708 |
| 4 | 0.0864 |
| 5 | 0.181 |
| 6 | 0.181 |
| 7 | 0.0516 |
| 8 | 0.416 |
| 9 | 0.161 |
| 10 | 0.0422 |
| 11 | 0.269 |
| 12 | 0.366 |
| 13 | 0.0879 |
| 14 | 0.105 |
| 15 | 0.201 |
| 16 | 0.0259 |
| 17 | 0.240 |
| 18 | 0.361 |
| 19 | 0.176 |
| 20 | 0.205 |
| 21 | 0.169 |
| 22 | <0.0195 |
| 23 | 0.0661 |
| 24 | 0.0831 |
| 25 | 0.122 |
| 26 | *0.0316 |
| 27 | <0.0195 |
| 28 | 0.0779 |
| 29 | 0.0274 |
| 30 | 0.0584 |
| 31 | *0.0254 |
| 32 | 0.0455 |
| 33 | *0.0261 |
| 34 | 0.0407 |
| 35 | 0.0681 |
| 36 | 0.0332 |
| 37 | 0.172 |
| 38 | 0.0962 |
| 39 | 0.309 |
| 40 | 0.0751 |
| 41 | 0.0834 |
| 42 | 0.0475 |
| 43 | 0.477 |
| 44 | 0.325 |
| 45 | 0.489 |
| 46 | 0.292 |
| 47 | 0.0627 |
| 48 | 0.0548 |
| 49 | 0.258 |
| 50 | 0.191 |
| 51 | 0.169 |
| 52 | 0.0464 |
| 53 | 0.428 |
| 54 | 0.0907 |
| 55 | 0.287 |
| 56 | 0.121 |
| 57 | 0.110 |
| 58 | 0.109 |
| 59 | 0.0825 |
| 60 | 0.231 |
| 61 | 0.0889 |
| 62 | 0.0780 |
| 63 | 0.202 |
| 64 | *0.0597 |
| 65 | 0.359 |
| 66 | 0.0436 |
| 67 | 0.0323 |
| 68 | 0.0412 |
| 69 | 0.715 |
| 70 | 0.182 |
| 71 | 0.0943 |
| 72 | 0.172 |
| 73 | 0.210 |
| 74 | 0.0476 |
| 75 | 0.138 |
| 76 | 0.116 |
| 77 | 0.0511 |
| 78 | 0.224 |
| 79 | 0.218 |
| 80 | 0.0262 |
| 81 | 0.0598 |
| 82 | 0.255 |
| 83 | 0.0461 |
| 84 | <0.0195 |
| 85 | 0.434 |
| 86 | 0.281 |
| 87 | 0.150 |
| 88 | 0.144 |
| 89 | 0.103 |
| 90 | 0.151 |
| 91 | 0.0440 |
| 92 | 0.170 |
| 93 | 0.110 |
| 94 | 0.0323 |
| 95 | 0.0916 |
| 96 | <0.0195 |
| 97 | 0.121 |
| 98 | 0.0745 |
| 99 | 0.261 |
| 100 | 0.0931 |
| 101 | 0.182 |
| 102 | 0.161 |
| 103 | 0.0407 |
| 104 | 0.240 |
| 105 | 0.0472 |
| 106 | 0.0999 |
| 107 | 0.123 |
| 108 | 0.103 |
| 109 | 0.192 |
| 110 | 0.345 |
| 111 | 0.0396 |
| 112 | 0.0355 |
| 113 | 0.147 |
| 114 | 0.221 |
| 115 | 0.0773 |
| 116 | 0.0830 |
| 117 | 0.0267 |
| 118 | <0.0195 |
| 119 | 0.257 |
| 120 | 0.107 |
| 121 | 0.115 |
| 122 | 0.112 |
| 123 | 0.287 |
| 124 | 0.0457 |
| 125 | 0.0826 |
| 126 | 0.122 |
| 127 | 0.0408 |
| 128 | 0.192 |
| 129 | 0.368 |
| 130 | 0.0976 |
| 131 | 0.0532 |
| 132 | 0.142 |
| 133 | 0.284 |
| 134 | 0.539 |
| 135 | 0.0345 |
| 136 | 0.139 |
| 137 | 0.113 |
| 138 | 0.233 |
| 139 | 0.117 |
| 140 | 0.311 |
| 141 | 0.120 |
| 142 | 0.129 |
| 143 | 0.0777 |
| 144 | 0.0978 |
| 145 | 0.162 |
| 146 | 0.150 |
| 147 | 0.328 |
| 148 | 0.166 |
| 149 | 0.135 |
| 150 | 0.350 |
| 151 | 0.152 |
| 152 | 0.463 |
| 153 | 0.306 |
| 154 | 0.236 |
| 155 | 0.307 |
| 156 | 0.394 |
| 157 | 0.185 |
| 158 | 0.335 |
| 159 | 0.166 |
| 160 | 0.278 |
| 161 | 0.102 |
| 162 | 0.266 |
| 163 | 0.0657 |
| 164 | 0.0885 |
| 165 | 0.171 |
| 166 | 0.260 |
| 167 | 0.149 |
| 168 | 0.179 |
| 169 | *0.0394 |
| 170 | 0.0451 |
| 171 | 0.0591 |
| 172 | 0.110 |
| 173 | 0.0769 |
| 174 | 0.110 |
| 175 | 0.226 |
| 176 | 0.223 |
| 177 | 0.286 |
| 178 | 0.192 |
| 179 | 0.0681 |
| 180 | 0.273 |
| 181 | 0.267 |
| 182 | 0.306 |
| 183 | <0.0195 |
| 184 | 0.0887 |
| 185 | 0.0775 |
| 186 | 0.0470 |
| 187 | 0.220 |
| 188 | 0.184 |
| 189 | 0.121 |
| 190 | 0.189 |
| 191 | 0.229 |
| 192 | 0.252 |
| 193 | 0.150 |
| 194 | 0.355 |
| 195 | 0.154 |
| 196 | 0.362 |
| 197 | 0.110 |
| 198 | 0.367 |
| 199 | 0.375 |
| 200 | 0.367 |
| 201 | 0.207 |
| 202 | 0.176 |
| 203 | 0.168 |
| 204 | 0.0704 |
| 205 | 0.0926 |
| 206 | 0.195 |
| 207 | 0.0890 |
| 208 | 0.121 |
| 209 | 0.139 |
| 210 | 0.124 |
| 211 | 0.0540 |
| 212 | 0.168 |
| 213 | 0.0876 |
| 214 | 0.0847 |
| 215 | 0.0795 |
| 216 | 0.185 |
| 217 | 0.180 |
| 218 | 0.0963 |
| 219 | 0.170 |
| 220 | 0.142 |
| 221 | 0.152 |
| 222 | 0.171 |
| 223 | 0.134 |
| 224 | 0.0652 |
| 225 | 0.0402 |
| 226 | 0.146 |
| 227 | 0.482 |
| 228 | 0.289 |
| 229 | 0.183 |
| 230 | 0.169 |
| 231 | 0.800 |
| 232 | 0.642 |
| 233 | 0.390 |
| 234 | 0.794 |
| 235 | 0.345 |
| 236 | 1.06 |
| 237 | 0.526 |
| 238 | 0.429 |
| 239 | 0.290 |
| 240 | 0.272 |
| 241 | 0.792 |
| 242 | 0.489 |
| 243 | 0.401 |
| 244 | 0.511 |
| 245 | 0.539 |
| 246 | 0.407 |
| 247 | 0.222 |
| 248 | 0.362 |
| 249 | 0.183 |
| 250 | 0.277 |
| 251 | 0.303 |
| 252 | 0.386 |
| 253 | 0.401 |
| 254 | 0.418 |
| 255 | 0.365 |
| 256 | 0.385 |
| 257 | 0.426 |
| 258 | 0.410 |
| 259 | 0.466 |
| 260 | 0.473 |
| 261 | 0.892 |
| 262 | 0.752 |
| 263 | 0.671 |
| 264 | 0.528 |
| 265 | 0.408 |
| 266 | 0.341 |
| 267 | 0.698 |
| 268 | 0.186 |
| 269 | 0.255 |
| 270 | 0.413 |
| 271 | 0.303 |
| 272 | 0.231 |
| 273 | 0.172 |
| 274 | 0.214 |
| 275 | 0.334 |
| 276 | 0.458 |
| 277 | 0.581 |
| 278 | 0.601 |
| 279 | 0.834 |
| 280 | 0.224 |
| 281 | 0.198 |
| 282 | 0.217 |
| 283 | 0.286 |
| 284 | 0.279 |
| 285 | 0.286 |
| 286 | 0.615 |
| 287 | 0.477 |
| 288 | 0.341 |
| 289 | 0.377 |
| 290 | 0.445 |
| 29 | 0.300 |
| 292 | 0.221 |
| 293 | 0.277 |
| 294 | 0.141 |
| 295 | 1.05 |
| 296 | 0.195 |
| 297 | 0.429 |
| 298 | 0.403 |
| 299 | 0.351 |
| 300 | 0.455 |
| 301 | 0.474 |
| 302 | 0.602 |
| 303 | 0.382 |
| 304 | 0.374 |
| 305 | 0.320 |
| 306 | 0.269 |
| 307 | 0.418 |
| 308 | 0.366 |
| 309 | 0.586 |
| 310 | 0.731 |
| 311 | 1.13 |
| 312 | 0.261 |
| 313 | 0.392 |
| 314 | 0.744 |
| 315 | 0.304 |
| 316 | 0.207 |
| 317 | 0.144 |
| 318 | 0.806 |
| 319 | 0.169 |
| 320 | 0.466 |
| 321 | 0.117 |
| 322 | *0.170 |
| 323 | 0.109 |
| 324 | 0.158 |
| 325 | 0.226 |
| 326 | 0.187 |
| 327 | 0.522 |
| 328 | 0.400 |
| 329 | 0.132 |
| 330 | 0.477 |
| 331 | 0.181 |
| 332 | 0.418 |
| 333 | 0.265 |
| 334 | 0.238 |
| 335 | 0.282 |
| 336 | 0.443 |
| 337 | 0.288 |
| 338 | 0.218 |
| 339 | 0.235 |
| 340 | 0.108 |
| 341 | 0.116 |
| 342 | 0.544 |
| 343 | 0.240 |
| 344 | 0.164 |
| 345 | 0.625 |
| 346 | 0.515 |
| 347 | 0.474 |
| 348 | 0.274 |
| 349 | 0.276 |
| 350 | 0.0925 |
| 351 | 0.194 |
| 352 | 0.222 |
| 353 | 0.0878 |
| 354 | 1.43 |
| 355 | 0.181 |
| 356 | 0.202 |
| 357 | 0.380 |
| 358 | 0.195 |
| 359 | 0.607 |
| 360 | 0.352 |
| 361 | 0.430 |
| 362 | 0.0803 |
| 363 | 0.100 |
| 364 | 0.363 |
| 365 | 0.281 |
| 366 | 0.529 |
| 367 | 0.192 |
| 368 | 0.469 |
| 369 | 0.0705 |
| 370 | 0.329 |
| 371 | 0.303 |
| 372 | 0.328 |
| 373 | 0.140 |
| 374 | 0.185 |
| 375 | 0.0389 |
| 376 | 0.114 |
| 377 | 0.159 |
| 378 | 0.183 |
| 379 | 0.168 |
| 380 | 0.180 |
| 381 | 0.173 |
| 382 | 0.373 |
| 383 | 0.603 |
| 384 | 0.516 |
| 385 | 0.344 |
| 386 | 0.388 |
| 387 | 0.450 |
| 388 | 0.613 |
| 389 | 0.599 |
| 390 | 0.473 |
| 391 | 0.339 |
| 392 | 0.282 |
| 393 | 0.710 |
| 394 | 0.568 |
| 395 | 0.601 |
| 396 | 0.451 |
| 397 | 0.200 |
| 398 | 0.399 |
| 399 | 0.181 |
| 400 | 0.436 |
| 401 | 0.472 |
| 402 | 0.146 |
| 403 | 0.682 |
| 404 | 0.437 |
| 405 | 0.349 |
| 406 | 0.298 |
| 407 | 0.590 |
| 408 | 0.179 |
| 409 | 0.363 |

The compounds provided herein demonstrate activity against A. baumannii, including A. baumannii that is multi-drug resistant. Because of the activity in multidrug resistant strains of A. baumannii, these compounds possess properties not found by other compounds in the same class. The compounds provided herein also possess limited off-target activity as well as decreased cytotoxicity. Accordingly, the compounds possess properties not found in previous compounds targeting A. baumannii.

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A compound of formula (I) or (II): 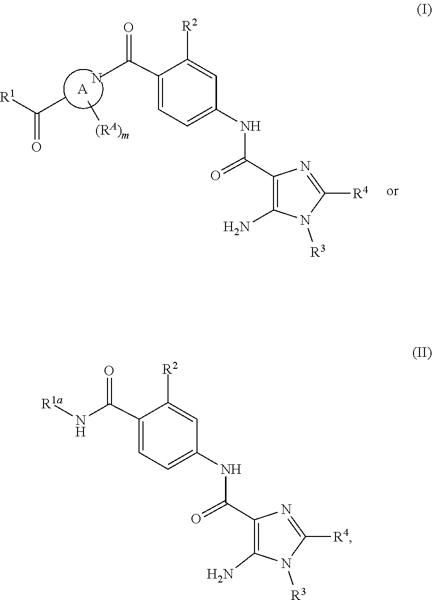
or a stereoisomer or pharmaceutically acceptable salt thereof, wherein
Ring A is 4- to 8-membered R^{A}-substituted or unsubstituted heterocyclyl;
R^{A} is halogen, NH₂, OH, unsubstituted C₁₋₃alkyl, or unsubstituted C₁₋₃haloalkyl;
m is 0 or 1;
R¹ is N(R⁶)₂, R⁶-substituted or unsubstituted C₁₋₆alkyl, R⁶-substituted or unsubstituted C₁₋₃haloalkyl, R⁶-substituted or unsubstituted C₃₋₆cycloalkyl, or R⁶-substituted or unsubstituted 4- to 8-membered heterocyclyl comprising 1 or 2 heteroatoms independently selected from N, O, S, or SO₂;
each R⁶ is independently hydrogen, halogen, CH(═NH)(NH₂), (═NH), N(R⁷)₂, OR⁷, NHC(O)R⁷, C(O)OR⁷, R⁷-substituted or unsubstituted C₁₋₆alkyl, R⁷-substituted or unsubstituted C₁₋₆haloalkyl, R⁷-substituted or unsubstituted C₃₋₆ cycloalkyl, or R⁷-substituted or unsubstituted 4- to 6-membered heterocyclyl; or
wherein two or more R⁶ groups together form a C₁₋₂ bridge;
each R⁷ is hydrogen, halo, OH, COOH, (═NH), NH₂, unsubstituted C₁₋₃alkyl, unsubstituted C₁₋₃alkyoxy, unsubstituted C₁₋₃alkylaryl, unsubstituted C₁₋₆alkyl heterocyclyl, or unsubstituted 4- to 6-membered heterocyclyl;
R^{1a} is a moiety of formula: 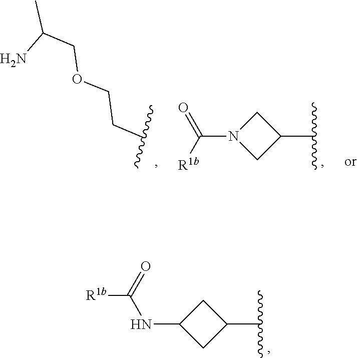
or a stereoisomer thereof;
R^{1b} is O—C₁₋₃alkyaryl, unsubstituted 5- to 7-membered heterocyclyl;
R² is hydrogen, halogen, unsubstituted C₁₋₃alkyl, or unsubstituted C₁₋₃haloalkyl;
R³ is a moiety of formula: 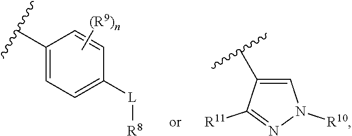
or a stereoisomer thereof
L is absent, —O—, or —NR^{8b}—;
R⁸ is R^{8a}-substituted or unsubstituted C₁₋₆alkyl, R^{8a}-substituted or unsubstituted C₁₋₃haloalkyl, or R^{8a}-substituted or unsubstituted C₃₋₆cycloalkyl;
wherein when L is absent R⁸ is 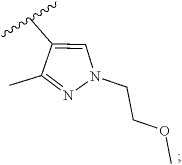
R^{8a} is unsubstituted C₁₋₃alkyl, OH, or OR^{8b} wherein R^{8b} is unsubstituted C₁₋₃alkyl;
R^{8b} is hydrogen or unsubstituted C₁₋₃alkyl;
R⁹ is halo, CN, unsubstituted C₁₋₆alkyl, unsubstituted C₁₋₃haloalkyl, unsubstituted C₁₋₆alkoxy, or unsubstituted cyclopropyl;
n is 0, 1, or 2;
R¹⁰ is R^{10A}-substituted or unsubstituted C₁₋₆alkyl, R^{10A}-substituted or unsubstituted C₂₋₆alkenyl, R^{10A}-substituted or unsubstituted C₂₋₆alkynl, R^{10A}-substituted or unsubstituted C₁₋₃haloalkyl, or R^{10A}-substituted or unsubstituted C₃₋₆cycloalkyl;
R^{10A} is halo, CN, OH, OCH₃, unsubstituted C₁₋₃haloalkyl, or R^{10B}-substituted or unsubstituted C₃₋₅cycloalkyl, or wherein 2 R^{10A} groups together form a R^{10B}-substituted or unsubstituted C₃₋₄spirocycle;
R^{10B} is halo or unsubstituted C₁₋₃haloalkyl;
R¹¹ is halo or unsubstituted C₁₋₃haloalkyl; and
R⁴ is hydrogen, halo, or unsubstituted C₁₋₃alkyl.

2. The compound of claim 1 wherein the compound comprises formula (I) and wherein
Ring A is 4- to 8-membered R^{A}-substituted or unsubstituted heterocyclyl;
R^{A} is halogen, NH₂, OH, unsubstituted C₁₋₃alkyl, or unsubstituted C₁₋₃haloalkyl;
m is 0 or 1;
R¹ is N(R⁶)₂, OR⁶, R⁶-substituted or unsubstituted C₁₋₆alkyl, R⁶-substituted or unsubstituted C₁₋₃haloalkyl, R⁶-substituted or unsubstituted C₃₋₆cycloalkyl, or R⁶-substituted or unsubstituted 4- to 9-membered heterocyclyl comprising 1 or 2 heteroatoms independently selected from N, O, S, or SO₂;
each R⁶ is independently hydrogen, halogen, CN, oxo, C(═NH)(NH₂), C(═NH)R⁷, (═NH), N(R⁷)₂, OR⁷, NHC(O)R⁷, C(O)OR⁷, S(O)(═NH)R⁷, R⁷-substituted or unsubstituted C₁₋₆alkyl, R⁷-substituted or unsubstituted C₁₋₆haloalkyl, R⁷-substituted or unsubstituted C₃₋₆ cycloalkyl, or R⁷-substituted or unsubstituted 4- to 6-membered heterocyclyl; or
wherein two or more R⁶ groups together form a C₁₋₂ bridge;
each R⁷ is hydrogen, halo, SO₂CH₃, OR^{7a}, COOH, (CH₂)ᵣOH, CH(CH₂OH)₂, (═NH), NH₂, R^{7a}-substituted or unsubstituted C₁₋₃alkyl, unsubstituted C₁₋₃alkyoxy, unsubstituted C₁₋₃alkylaryl, unsubstituted C₁₋₆alkyl heterocyclyl, or unsubstituted 4- to 8-membered heterocyclyl;
each R^{7a} is independently SO₂CH₃, SO₂CH₃, OH, (CH₂)ᵣOH, NH₂, NHCH₃, N(CH₃)₂, NHSO₂CH₃, unsubstituted C₁₋₃ alkyl, unsubstituted tetrahydropyrimidinyl, unsubstituted oxetanyl, unsubstituted pyrrolidinyl, unsubstituted piperidinyl, unsubstituted oxooxazolidinyl, or unsubstituted imidazolyl;
r is 1, 2, or 3;
R² is hydrogen, halogen, unsubstituted C₁₋₃alkyl, or unsubstituted C₁₋₃haloalkyl;
R³ is a moiety of formula: 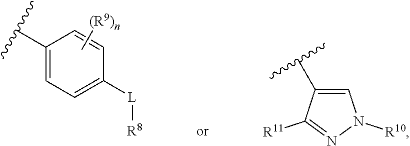
or a stereoisomer thereof
L is absent, —O—, or —NR^{8b}—;
R⁸ is R^{8a}-substituted or unsubstituted C₁₋₆alkyl, R^{8a}-substituted or unsubstituted C₁₋₃haloalkyl, or R^{8a}-substituted or unsubstituted C₃₋₆cycloalkyl;
wherein when L is absent R⁸ is 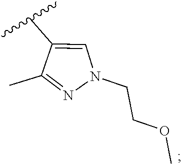
R^{8a} is unsubstituted C₁₋₃alkyl, OH, or OR^{8b} wherein R^{8b} is unsubstituted C₁₋₃alkyl;
R^{8b} is hydrogen or unsubstituted C₁₋₃alkyl;
R⁹ is halo, CN, unsubstituted C₁₋₆alkyl, unsubstituted C₁₋₃haloalkyl, unsubstituted C₁₋₆alkoxy, or unsubstituted cyclopropyl;
n is 0, 1, or 2;
R¹⁰ is R^{10A}-substituted or unsubstituted C₁₋₆alkyl, R^{10A}-substituted or unsubstituted C₂₋₆alkenyl, R^{10A}-substituted or unsubstituted C₂₋₆alkynl, R^{10A}-substituted or unsubstituted C₁₋₃haloalkyl, or R^{10A}-substituted or unsubstituted C₃₋₆cycloalkyl;
R^{10A} is halo, CN, OH, OCH₃, unsubstituted C₁₋₃haloalkyl, or R^{10B}-substituted or unsubstituted C₃₋₅cycloalkyl, or wherein 2 R^{10A} groups together form a R^{10B}-substituted or unsubstituted C₃₋₄spirocycle;
R¹¹ is halo or unsubstituted C₁₋₃haloalkyl; and
R⁴ is hydrogen, halo, or unsubstituted C₁₋₃alkyl.

3. The compound of claim 2, wherein Ring A is a moiety of formula: 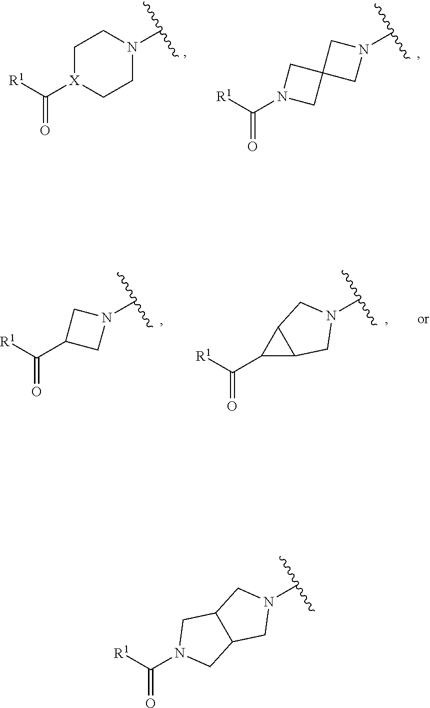
or a stereoisomer thereof, wherein
X is N or CR^{x};
R^{x} is hydrogen or halo; and
p is an integer of 0, 1, 2, 3, or 4.

4. The compound of claim 2, wherein the compound of formula (I) comprises formula (Ia): 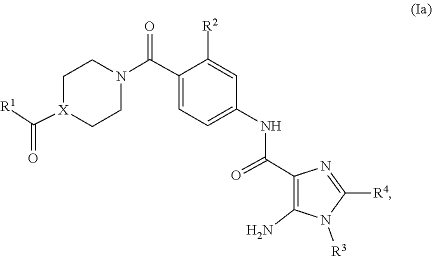
or a stereoisomer or pharmaceutically acceptable salt thereof, wherein
X is N, CH or CF.

5. (canceled)

6. (canceled)

7. The compound of claim 2, wherein R¹ is a moiety of formula: 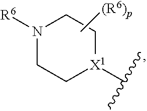
or a stereoisomer thereof, wherein X¹ is N or CH.

8. The compound of claim 2, wherein R¹ is a moiety of formula: 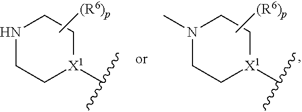
or a stereoisomer thereof, wherein X¹ is N or CH.

9.-16. (canceled)

17. The compound of claim 2, wherein R⁶ is halo.

18. (canceled)

19. (canceled)

20. The compound of claim 2, wherein R¹ is a moiety of formula: 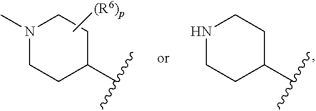
or a stereoisomer thereof, wherein R⁶ is oxo or halo (F), and p is 1 or 2.

21.-30. (canceled)

31. The compound of claim 1 having formula (II).

32.-34. (canceled)

35. The compound of claim 1, wherein R³ is a moiety of formula: 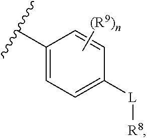
or a stereoisomer thereof.

36.-41. (canceled)

42. The compound of claim 35, wherein R³ is a moiety of formula: 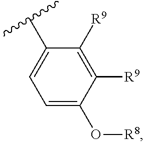
or a stereoisomer thereof, wherein R⁸ is CH₂F, CHF₂, CH₃, unsubstituted cyclopropyl.

43. (canceled)

44. The compound of claim 1, wherein R³ is a moiety of formula: 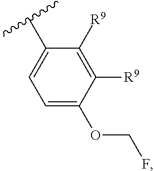
or a stereoisomer thereof, wherein R⁹ is independently halo, CH₃, CF₃, CH₂F, or CHF₂.

45. (canceled)

46. (canceled)

47. The compound of claim 44, wherein R⁹ is F.

48. The compound of claim 1, wherein R³ is a moiety of formula: 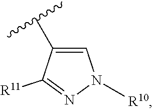
or a stereoisomer thereof, wherein R¹¹ is CF₃.

49. (canceled)

50. The compound of claim 48, wherein R¹⁰ is R^{10A}-substituted or unsubstituted C₁₋₆alkyl, R^{10A}-substituted or unsubstituted C₂₋₆alkenyl, or R^{10A}-substituted or unsubstituted C₂₋₆alkynl.

51.-53. (canceled)

54. The compound of claim 48, wherein R³ is a moiety of formula: 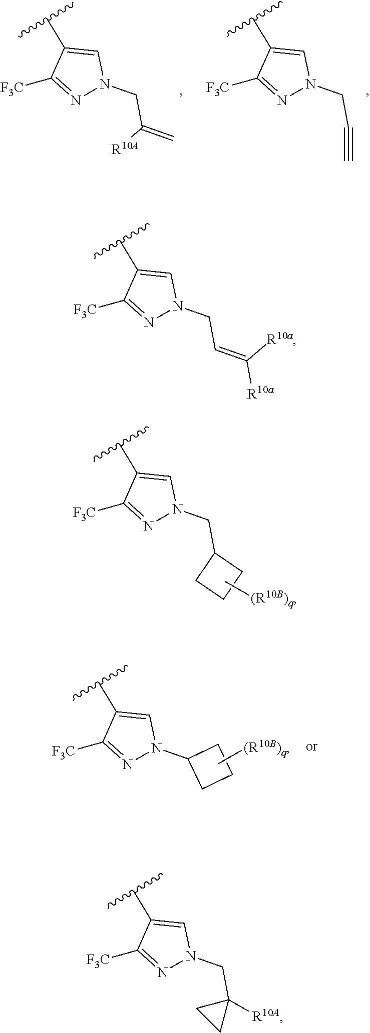
or a stereoisomer thereof, wherein q is an integer of 0, 1, or 2.

55. (canceled)

56. (canceled)

57. The compound of claim 1, wherein R² is hydrogen.

58. The compound of claim 1, wherein R² is unsubstituted C₁₋₃alkyl.

59. The compound of claim 1, wherein R² is halo.

60.-63. (canceled)

64. The compound of claim 1, wherein R⁴ is hydrogen.

65. The compound of claim 1, wherein R⁴ is halo.

66. (canceled)

67. The compound of claim 1, wherein R⁴ is CH₃.

68. The compound of claim 2, wherein the compound of formula (I) has formula: 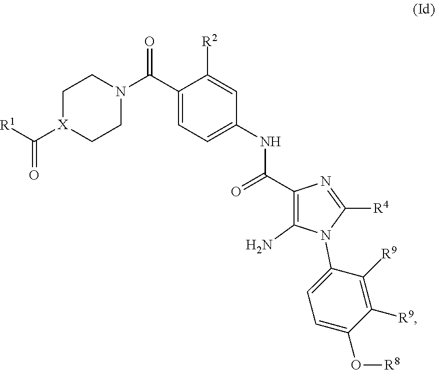
wherein X is N or CH.

69. (canceled)

70. The compound of claim 1, wherein the compound of formula (I) has formula: 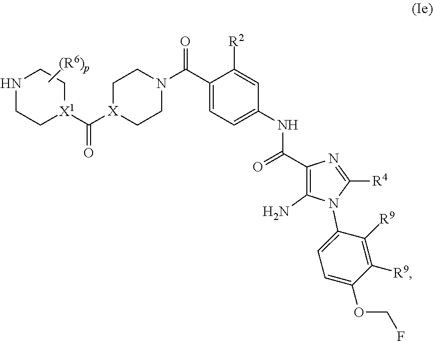
wherein,
X is N or CH;
X¹ is CH;
R² is halo; and
R⁴ is hydrogen.

71. A compound of Table 1 or a stereoisomer or pharmaceutically acceptable salt thereof.

72. A pharmaceutical composition comprising a compound or a pharmaceutically acceptable salt thereof of claim 1 and one or more pharmaceutically acceptable excipients.

73.-77. (canceled)

78. A method of treating an infection caused by Gram-negative bacteria in a patient having such an infection, the method comprising administering to the patient an effective amount of a compound of claim 1 or a stereoisomer, tautomer, or pharmaceutically acceptable salt thereof.

79. The method of claim 78, wherein the Gram-negative bacteria are Klebsiella pneumoniae, Acinetobacter baumannii, Pseudomonas aeruginosa, Enterobacter species or E. coli.

80. The method of claim 78, wherein the Gram-negative bacteria are Acinetobacter baumannii.

81. A method of treating an infection caused by Enterococcus faecium, Staphylococcus aureus, Klebsiella pneumoniae, Acinetobacter baumannii, Pseudomonas aeruginosa, Enterobacter species or E. coli, or a combination thereof, in a patient having such infection, the method comprising administering to the patient a compound of claim 1 or a stereoisomer, tautomer or pharmaceutically acceptable salt thereof.
